(19) United States
(12) Patent Application Publication
(10) Pub. No.: US 2024/0150321 A1
(43) Pub. Date: May 9, 2024

(54) PYRAZOLYLSULFONAMIDE COMPOUNDS AND THEIR USE IN THERAPY

(71) Applicant: HOTSPOT THERAPEUTICS, INC., Boston, MA (US)

(72) Inventors: Yingzhi Bi, Plainsboro, NJ (US); Kenneth G. Carson, Princeton, NJ (US); Geraldine Cirillo Harriman, Charlestown, RI (US); Sundarapandian Thangapandian, Foxboro, MA (US); Christian Josef Kuper, Hertford (GB); Sebastien Louis Degorce, Saffron Walden (GB); Benjamin Paul Whittaker, Potton (GB); Arwel Lewis, Royston (GB); Maria Angel Palomero-Vazquez, Whittlesford (GB)

(21) Appl. No.: 18/455,767

(22) Filed: Aug. 25, 2023

Related U.S. Application Data
(60) Provisional application No. 63/401,490, filed on Aug. 26, 2022.

(51) Int. Cl. C07D 401/14; C07D 403/14; C07D 407/14; C07D 413/14; C07D 471/04; C07D 487/06; C07D 498/04

(52) U.S. Cl. CPC C07D 401/14; C07D 403/14; C07D 407/14; C07D 413/14; C07D 471/04; C07D 487/06; C07D 498/04

(57) ABSTRACT

The invention provides pyrazolylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder.

### CROSS REFERENCE TO RELATED APPLICATIONS

This application claims the benefit of and priority to U.S. Provisional Patent Application Ser. No. 63/401,490, filed Aug. 26, 2022; the contents of which are hereby incorporated by reference in their entirety.

### FIELD of THE INVENTION

The invention provides pyrazolylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. Moreover, new therapies that achieve an anti-cancer effect through a different mechanism present an opportunity to treat cancers more effectively and/or to treat cancers that have become resistant to currently available medicines.

Inflammatory disorders impact a substantial number of patients and often involve situations where the patient's biological response to a stimulus results in the immune system attacking the body's own cells or tissues. This can lead to abnormal inflammation and result in chronic pain, redness, swelling, stiffness, and/or damage to normal tissues. Current treatment options for these inflammatory disorders are not effective for all patients and/or can have substantial adverse side effects.

Human mucosa-associated lymphoid tissue protein 1 (MALT1) is a key regulator of immune responses and is an immune modulatory target for the treatment of autoimmune and inflammatory diseases. In addition, research indicates that MALT1 inhibition impairs immune suppressive function of regulatory T cells in a tumor microenvironment, implicating MALT1 inhibitors for boosting anti-tumor immunity in the treatment of solid cancers. See, for example, Isabel Hamp et al. in Expert Opinion on Therapeutic Patents (2021) vol. 12, pages 1079-1096.

The present invention addresses the foregoing needs and provides other related advantages.

### SUMMARY

The invention provides pyrazolylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. In particular, one aspect of the invention provides a collection of pyrazolylsulfonamide compounds, such as a compound represented by Formula I:

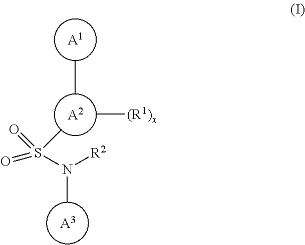

or a pharmaceutically acceptable salt thereof, where the variables are as defined in the detailed description. Further description of additional collections of pyrazolylsulfonamide compounds are described in the detailed description. The compounds may be part of a pharmaceutical composition comprising a pharmaceutically acceptable carrier.

Another aspect of the invention provides a collection of pyrazolylsulfonamide compounds, such as a compound represented by Formula II:

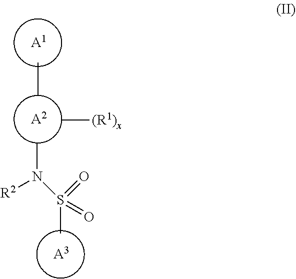

or a pharmaceutically acceptable salt thereof, where the variables are as defined in the detailed description. Further description of additional collections of pyrazolylsulfonamide compounds are described in the detailed description. The compounds may be part of a pharmaceutical composition comprising a pharmaceutically acceptable carrier.

Another aspect of the invention provides a method of treating a disease or condition mediated by MALT1 in a subject. The method comprises administering a therapeutically effective amount of a compound described herein, such as a compound of Formula I, I-1, I-2, I-3, I-4, or II, or other compounds in Section I to a subject in need thereof to treat the disease or condition, as further described in the detailed description.

Another aspect of the invention provides a method of inhibiting the activity of MALT1. The method comprises contacting a MALT1 with an effective amount of a compound described herein, such as a compound of Formula I, I-1, I-2, I-3, I-4, or II, or other compounds in Section I to inhibit the activity of said MALT1, as further described in the detailed description

### DETAILED DESCRIPTION

The invention provides pyrazolylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. The practice of the present invention employs, unless otherwise indicated, conventional techniques of organic chemistry, pharmacology, molecular biology (including recombinant techniques), cell biology, biochemistry, and immunology. Such techniques are explained in the literature, such as in Comprehensive Organic Synthesis (B. M. Trost & I. Fleming, eds., 1991-1992); Handbook of Experimental Immunology (D. M. Weir & C. C. Blackwell, eds.); Current Protocols in Molecular Biology (F. M. Ausubel et al., eds., 1987, and periodic updates); and Current Protocols in Immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

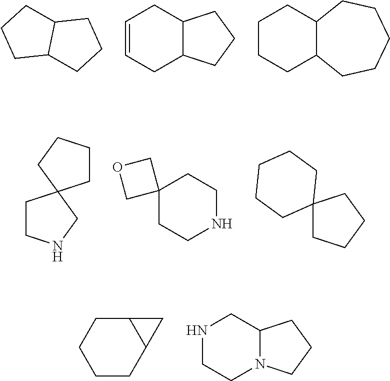

Exemplary bridged bicyclics include:

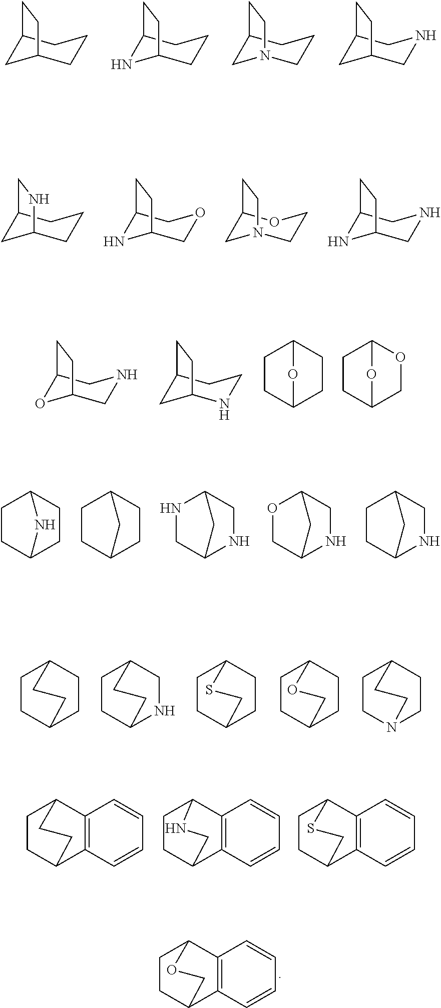

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” or “halo” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

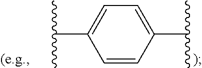 [SMILES: *c1ccc(*)cc1]

“phenylene” is a trivalent phenyl group when it has three groups attached to it

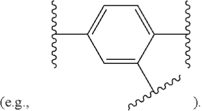 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to “ne o’ more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it

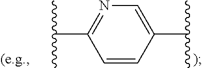 [SMILES: *c1ccc(*)nc1]

“pyridinylene” is a trivalent pyridine radical when it has three groups attached to it

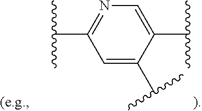 [SMILES: *c1cc(*)c(*)cn1]

The term “pyrazolylene” refers to a multivalent pyrazole radical having the appropriate number of open valences to account for groups attached to it. For example, “pyrazolylene” is a bivalent pyrazole radical when it has two groups attached to it

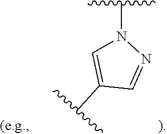 [SMILES: *c1cnn(*)c1]

The term “1,2,3-triazolylene” refers to a multivalent 1,2,3-triazole radical having the appropriate number of open valences to account for groups attached to it. For example, “1,2,3-triazolylene” is a bivalent 1,2,3-triazole radical when it has two groups attached to it

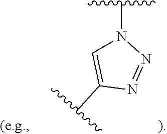 [SMILES: *c1cn(*)nn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OsiR^{∘}₃; —(CH₂)₀₋₄₀C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{●} selected from ═O and ═S; or each R^{●} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OsiR^{●}₃, —C(O)SR^{●}, —(C₁-4 straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R*is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R^{●} is C₁₋₆ aliphatic, R^{●} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R is independently hydrogen, C₁₋₆ aliphatic, unsubstituted —Oph, or an unsubstituted 5-6-membered saturated, partially unsaturated, or aryl ring having 0-4 heteroatoms independently selected from nitrogen, oxygen, or sulfur, or, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences 1977, 66(1), 1-19; P. Gould, International J. of Pharmaceutics 1986, 33, 201-217; Anderson et al., The Practice ofMedicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within +10% of the stated value. The invention encompasses embodiments where the value is within ±9%, ±8%, ±7%, ±6%, ±5%, ±4%, ±3%, ±2%, or ±1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-Methyl-1-pentyl, 2-methyl-2-pentyl, 3-methyl-2-pentyl, 4-Methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group. The term “haloalkenyl” refers to an alkenyl group that is substituted with at least one halogen.

The terms “alkenyl” and “alkynyl” are art-recognized and refer to unsaturated aliphatic groups analogous in length and possible substitution to the alkyls described above, but that contain at least one double or triple bond, respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group. The term “hydroxyalkoxyl” refers to an alkoxyl group that is substituted with at least one hydroxyl group. In certain embodiments, the hydroxyalkoxyl is an alkoxyl group that is substituted with one hydroxyl group.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, include humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate-buffered saline solution, water, emulsions (e.g., such as oil/water or water/oil emulsions), and various types of wetting agents. The compositions also can include stabilizers and preservatives. For examples of carriers, stabilizers and adjuvants, see e.g., Martin, Remington 's Pharmaceutical Sciences, 15^{th} Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Pyrazolylsulfonamide Compounds

The invention provides pyrazolylsulfonamide compounds. The compounds may be used in the pharmaceutical compositions and therapeutic methods described herein. Exemplary compounds are described in the following sections, along with exemplary procedures for making the compounds.

### Part A:

One aspect of the invention provides a compound represented by Formula I:

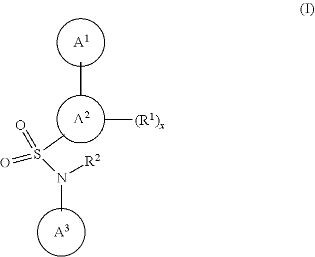

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2, or 3 heteratoms independently selected from oxygen, nitrogen, and sulfur, a 5-6 membered partially unsaturated heterocyclyl containing 1 nitrogen atom, or a 5-6 membered deuteroheteroaryl containing 1 nitrogen atom, wherein the heteroaryl, saturated heterocyclyl, and deuteroheteroaryl are substituted with n occurrences of R⁶, and wherein the partially unsaturated heterocyclyl is substituted with n occurrences of R⁶ and 1 occurrence of oxo;
A² is pyrazolylene or 1,2,3-triazolylene;
A³ is

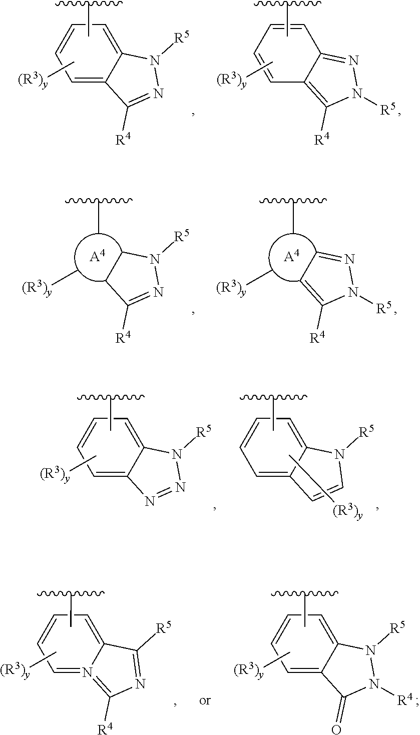

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, C₁₋₄ haloalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), C₁₋₆ hydroxyalkyl, —C(O)R¹⁰, —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, C₁₋₄ alkoxyl, C₁₋₄ alkyl, or deuterium;
R⁶ represents independently for each occurrence halo, hydroxyl, C₁₋₆ alkyl, C₁₋₆ haloalkyl, cyano, C₁₋₆ alkoxyl, C₁₋₆ hydroxyalkoxyl, —(C₃₋₇ cycloalkyl substituted with q occurrences of R¹²), —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, C₁₋₆ hydroxyalkyl, —(C₀₋₄ alkylene)-(C₁₋₆ alkoxyl), —(C₁₋₄ alkylene)-CN, C₂₋₄ alkenyl, C₂₋₄ haloalkenyl, —(C₀₋₄ alkylene)-(N(R¹³)(R¹⁴)), —(C₀₋₄ alkylene)-(3-7 membered saturated or partially unsaturated monocyclic heterocyclyl containing 1 or 2 heteroatoms independently selected from oxygen and nitrogen), or a 6-7 membered saturated bicyclic heterocyclyl containing 1 nitrogen atom, wherein the monocyclic heterocyclyl and bicyclic heterocyclyl are substituted with 0, 1, or 2 occurrences of R¹²;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, C₁₋₆ hydroxyalkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
R¹² represents independently for each occurrence C₁₋₆ alkyl, C₁₋₆ alkoxyl, halo, hydroxyl, C₁₋₆ haloalkyl, oxo, cyano, or —C(O)—(C₁₋₄ alkyl); or two R¹² groups taken together with the carbon atom to which they are attached form a 3-7 membered saturated carbocyclic ring;
R¹³ and R¹⁴ are independently hydrogen or C₁₋₄ alkyl;
n, m, x, and y are independently 0, 1, or 2; and
q is 0, 1, 2, or 3.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).′

In certain embodiments, the compound is a compound of Formula I.

As defined generally above, A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2, or 3 heteratoms independently selected from oxygen, nitrogen, and sulfur, a 5-6 membered partially unsaturated heterocyclyl containing 1 nitrogen atom, or a 5-6 membered deuteroheteroaryl containing 1 nitrogen atom, wherein the heteroaryl, saturated heterocyclyl, and deuteroheteroaryl are substituted with n occurrences of R⁶, and wherein the partially unstaturated heterocyclyl is substituted with n occurrences of R⁶ and 1 occurrence of oxo. In certain embodiments, A¹ is a 5-6 membered heteroaryl containing 1, 2, or 3 heteratoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl are substituted with n occurrences of R⁶. In certain embodiments, A¹ is a 5-6 membered heteroaryl containing 1, 2, or 3 heteratoms independently selected from oxygen, nitrogen, and sulfur, wherein the heteroaryl is substituted with n occurrences of R⁶. In certain embodiments, A¹ is a 6-membered heteroaryl containing 1, 2, or 3 heteratoms independently selected from oxygen, nitrogen, and sulfur, wherein the heteroaryl is substituted with n occurrences of R⁶. In certain embodiments, A¹ is pyridinyl, pyrimidinyl, pyridazinyl, or pyrazinyl, each of which is substituted with n occurrences of R⁶. In certain embodiments, A¹ is pyridinyl substituted with n occurrences of R⁶. In certain embodiments, A¹ is

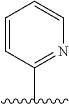 [SMILES: *c1ccccn1]

substituted with n occurrences of R⁶. In certain embodiments, A¹ is

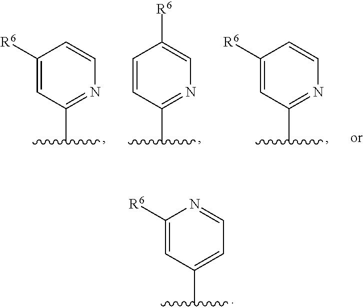

In certain embodiments, A¹ is

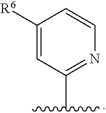

In certain embodiments, A¹ is

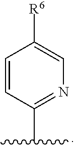

In certain embodiments, A¹ is

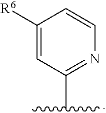

In certain embodiments, A¹ is

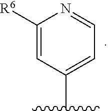

In certain embodiments, A¹ is is

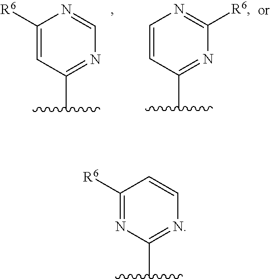

In certain embodiments, A¹ is

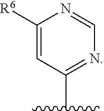

In certain embodiments, A¹ is

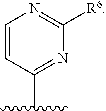

In certain embodiments, A¹ is

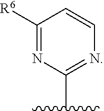

In certain embodiments, A¹ is a 5-membered heteroaryl containing 1, 2, or 3 heteratoms independently selected from oxygen, nitrogen, and sulfur, wherein the heteroaryl is substituted with n occurrences of R⁶. In certain embodiments, A¹ is a 9-10 membered bicyclic heteroaryl containing 1, 2, or 3 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 1 heteroatom selected from oxygen, nitrogen, and sulfur, wherein the heteroaryl is substituted with n occurrences of R⁶. In certain embodiments, A¹ is a 9-10 membered bicyclic heteroaryl containing 2 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 3 heteroatoms independently selected from oxygen, nitrogen, and sulfur, wherein the heteroaryl is substituted with n occurrences of R⁶. In certain embodiments, A¹ is a 3-10 membered saturated heterocyclyl containing 1, 2, or 3 heteroatoms independently selected from oxygen, nitrogen, and sulfur, wherein the saturated heterocyclyl is substituted with n occurrences of R⁶. In certain embodiments, A¹ is a 3-10 membered saturated heterocyclyl containing 1 heteroatom independently selected from oxygen, nitrogen, and sulfur, wherein the saturated heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the saturated heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the saturated heterocyclyl is substituted with n occurrences of R⁶. In certain embodiments, A¹ is a 5-6 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is a 5 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is a 6 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is a 5-6 membered deuteroheteroaryl containing 1 nitrogen atom, wherein the deuteroheteroaryl is substituted with n occurrences of R⁶. In certain embodiments, A¹ is a 5 membered deuteroheteroaryl containing 1 nitrogen atom, wherein the deuteroheteroaryl is substituted with n occurrences of R. In certain embodiments, A¹ is a 6 membered deuteroheteroaryl containing 1 nitrogen atom, wherein the deuteroheteroaryl is substituted with n occurrences of R⁶. In certain embodiments, A¹ is selected from the groups depicted in the compounds in Table 1 below.

As defined generally above, A² is pyrazolylene or 1,2,3-triazolylene. In certain embodiments, A² is pyrazolylene. In certain embodiments, A² is 1,2,3-triazolylene. In certain embodiments, A² is

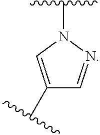 [SMILES: *c1cnn(*)c1]

In certain embodiments, A² is

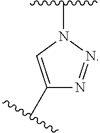 [SMILES: *c1cn(*)nn1]

In certain embodiments, A² is selected from the groups depicted in the compounds in Table 1 below.

As defined generally above, A³ is

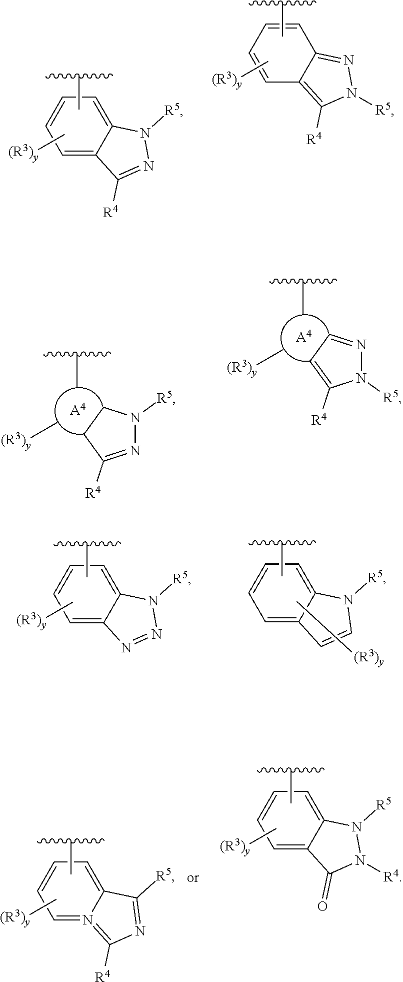

In certain embodiments, A³ is

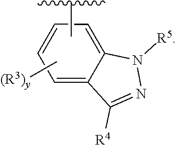

In certain embodiments, A³ is

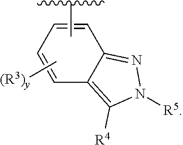

In certain embodiments, A³ is

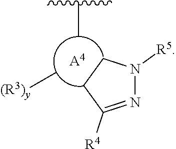

In certain embodiments, A³ is

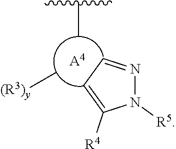

In certain embodiments, A³ is

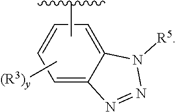

In certain embodiments, A³ is

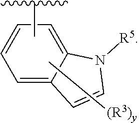

In certain embodiments, A³ is

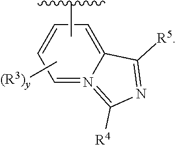

In certain embodiments, A³ is

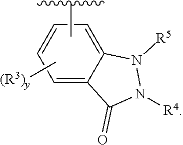

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R² is C₁. 4 alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, C₁₋₄ haloalkyl, —(C₁₋₄ alkylene)-(C₁₋₆ alkoxyl), or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ haloalkyl. In certain embodiments, R⁵ is C₁ haloalkyl. In certain embodiments, R⁵ is —CHF₂. In certain embodiments, R⁵ is C₂ haloalkyl. In certain embodiments, R⁵ is C₃ haloalkyl. In certain embodiments, R⁵ is C₄ haloalkyl. In certain embodiments, R⁵ is —(C₁₋₄ alkylene)-(C₁₋₆ alkoxyl). In certain embodiments, R⁵ is —CH₂CH₂OCH₃. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), C₁₋₆ hydroxyalkyl, —C(O)R¹⁰, —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R³ is C₁₋₆ alkyl. In certain embodiments, R³ is methyl. In certain embodiments, R³ is ethyl. In certain embodiments, R³ is C₃ alkyl. In certain embodiments, R³ is C₄ alkyl. In certain embodiments, R³ is C₅ alkyl. In certain embodiments, R³ is C₆ alkyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is C₂ alkoxyl. In certain embodiments, R³ is C₃ alkoxyl. In certain embodiments, R³ is C₄ alkoxyl. In certain embodiments, R³ is C₅ alkoxyl. In certain embodiments, R³ is C₆ alkoxyl. In certain embodiments, R³ is halo. In certain embodiments, R³ is hydroxyl In certain embodiments, R³ is C₁₋₆ haloalkyl. In certain embodiments, R³ is C₁ haloalkyl. In certain embodiments, R³ is C₂ haloalkyl. In certain embodiments, R³ is C₃ haloalkyl. In certain embodiments, R³ is C₄ haloalkyl. In certain embodiments, R³ is C₅ haloalkyl. In certain embodiments, R³ is C₆ haloalkyl. In certain embodiments, R³ is C₁₋₆ deuteroalkyl. In certain embodiments, R³ is C₁₋₆ deuteroalkoxyl. In certain embodiments, R³ is C₃₋₇ halocycloalkyl. In certain embodiments, R³ is C₃₋₇ hydroxycycloalkyl. In certain embodiments, R³ is —O—C₃₋₇ cycloalkyl. In certain embodiments, R³ is —(C₀₋₄ alkylene)-CN. In certain embodiments, R³ is cyano. In certain embodiments, R³ is C₂₋₄ alkynyl. In certain embodiments, R³ is —N(R⁸)(R⁹). In certain embodiments, R³ is C₁₋₆ hydroxyalkyl. In certain embodiments, R³ is C₁ hydroxyalkyl. In certain embodiments, R³ is C₂ hydroxyalkyl. In certain embodiments, R³ is —CH(CH₃)OH. In certain embodiments, R³ is C₃ hydroxyalkyl. In certain embodiments, R³ is C₄ hydroxyalkyl. In certain embodiments, R³ is C₅ hydroxyalkyl. In certain embodiments, R³ is C₆ hydroxyalkyl. In certain embodiments, R³ is —C(O)R¹⁰. In certain embodiments, R³ is —C(O)CH₃. In certain embodiments, R³ is CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, R³ is a 3-7 membered saturated heterocyclyl containing 1 or 2 heteroatoms independently selected from oxygen and nitrogen, wherein the heterocyclyl is substituted with 0 or 1 halo. In certain embodiments, R³ is a 3-7 membered saturated heterocyclyl containing 1 heteroatom selected from oxygen and nitrogen, wherein the heterocyclyl is substituted with 0 or 1 halo. In certain embodiments, R³ is a 3-7 membered saturated heterocyclyl containing 2 heteroatoms independently selected from oxygen and nitrogen, wherein the heterocyclyl is substituted with 0 or 1 halo. In certain embodiments, R³ is selected from the groups depicted in the compounds in Table 1 below.

As defined generally above, R⁴ is hydrogen, halo, C₁₋₄ alkoxyl, C₁₋₄ alkyl, or deuterium. In certain embodiments, R⁴ is hydrogen. In certain embodiments, R⁴ is halo. In certain embodiments, R⁴ is F. In certain embodiments, R⁴ is Cl. In certain embodiments, R⁴ is Br. In certain embodiments, R⁴ is I. In certain embodiments, R⁴ is C₁₋₄ alkoxyl. In certain embodiments, R⁴ is C₁ alkoxyl. In certain embodiments, R⁴ is C₂ alkoxyl. In certain embodiments, R⁴ is C₃ alkoxyl. In certain embodiments, R⁴ is C₄ alkoxyl. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is deuterium. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₁₋₆ hydroxyalkoxyl, —(C₃₋₇ cycloalkyl substituted with q occurrences of R¹²), —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R′)S(O₂)R¹⁰, C₁₋₆ hydroxyalkyl, —(C₁₋₄ alkylene)-(C₁₋₆ alkoxyl), —(C₁₋₄ alkylene)-CN, C₂₋₄ alkenyl, C₂₋₄ haloalkenyl, —(C₀₋₄ alkylene)-(N(R¹³)(R¹⁴)), —(C₀₋₄ alkylene)-(3-7 membered saturated or partially unsaturated monocyclic heterocyclyl containing 1 or 2 heteroatoms independently selected from oxygen and nitrogen), or a 6-7 membered saturated bicyclic heterocyclyl containing 1 nitrogen atom, wherein the monocyclic heterocyclyl and bicyclic heterocyclyl are substituted with 0, 1, or 2 occurrences of R¹². In certain embodiments, R⁶ is C₁₋₆ haloalkyl. In certain embodiments, R⁶ is —CF₃. In certain embodiments, R⁶ is C₁ haloalkyl. In certain embodiments, R⁶ is C₂ haloalkyl. In certain embodiments, R⁶ is C₃ haloalkyl. In certain embodiments, R⁶ is C₄ haloalkyl. In certain embodiments, R⁶ is C₅ haloalkyl. In certain embodiments, R⁶ is C₆ haloalkyl. In certain embodiments, R⁶ is C₁₋₆ alkyl. In certain embodiments, R⁶ is methyl. In certain embodiments, R⁶ is C₂ alkyl. In certain embodiments, R⁶ is C₃ alkyl. In certain embodiments, R⁶ is C₄ alkyl. In certain embodiments, R⁶ is C₅ alkyl. In certain embodiments, R⁶ is C₆ alkyl. In certain embodiments, R⁶ is C₃₋₇ cycloalkyl substituted with q occurrences of R¹². In certain embodiments, R⁶ is C₃₋₇ cycloalkyl. In certain embodiments, R⁶ is cyclopropyl. In certain embodiments, R⁶ is halo. In certain embodiments, R⁶ is hydroxyl. In certain embodiments, R⁶ is cyano. In certain embodiments, R⁶ is C₁₋₆ alkoxyl. In certain embodiments, R⁶ is C₁₋₆ alkoxyl substituted with 0, 1, or 2 occurrences of hydroxyl. In certain embodiments, R⁶ is C₁₋₆ alkoxyl substituted with 1 occurrence of hydroxyl. In certain embodiments, R⁶ is C₁₋₆ hydroxyalkoxyl. In certain embodiments, R⁶ is C₁ alkoxyl. In certain embodiments, R⁶ is C₂ alkoxyl. In certain embodiments, R⁶ is C₃ alkoxyl. In certain embodiments, R⁶ is C₄ alkoxyl. In certain embodiments, R⁶ is C₅ alkoxyl. In certain embodiments, R⁶ is C₆ alkoxyl. In certain embodiments, R⁶ is —O—C₃₋₇ cycloalkyl. In certain embodiments, R⁶ is —N(R⁸)(R⁹). In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is C₁₋₆ hydroxyalkyl. In certain embodiments, R⁶ is C₁ hydroxyalkyl. In certain embodiments, R⁶ is C₂ hydroxyalkyl. In certain embodiments, R⁶ is C₃ hydroxyalkyl. In certain embodiments, R⁶ is C₄ hydroxyalkyl. In certain embodiments, R⁶ is C₅ hydroxyalkyl. In certain embodiments, R⁶ is C₆ hydroxyalkyl. In certain embodiments, R⁶ is —(C₁₋₄ alkylene)-(C₁₋₆ alkoxyl). In certain embodiments, R⁶ is —(C₁₋₄ alkylene)-CN.

In certain embodiments, R⁶ is C₂₋₄ alkenyl substituted with 0 or 1 occurrences of halo. In certain embodiments, R⁶ is C₂₋₄ alkenyl. In certain embodiments, R⁶ is C₂₋₄ haloalkenyl. In certain embodiments, R⁶ is C₂₋₄ alkenyl substituted with 1 occurrence of fluoro. In certain embodiments, R⁶ is —(C₂₋₄ alkylene)-(N(R¹³)(R¹⁴)). In certain embodiments, R⁶ is —(N(R¹³)(R¹⁴)). In certain embodiments, R⁶ is —(C₁ alkylene)-(N(R¹³)(R¹⁴)). In certain embodiments, R⁶ is —(C₂ alkylene)-(N(R¹³)(R¹⁴)). In certain embodiments, R⁶ is —(C₃ alkylene)-(N(R¹³)(R¹⁴)). In certain embodiments, R⁶ is —(C₄ alkylene)-(N(R¹³)(R¹⁴)). In certain embodiments R⁶ is —(C₀₋₄ alkylene)-(3-7 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹²). In certain embodiments R⁶ is —(C₁ alkylene)-(3-7 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹²). In certain embodiments R⁶ is —(C₂ alkylene)-(3-7 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹²). In certain embodiments R⁶ is —(C₃ alkylene)-(3-7 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹²). In certain embodiments R⁶ is —(C₄ alkylene)-(3-7 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹²). In certain embodiments, R⁶ is a 3-7 membered saturated monocyclic heterocyclyl containing 1 or 2 heteroatoms independently selected from oxygen and nitrogen, wherein the monocyclic heterocyclyl is substituted with 0, 1, or 2 occurrences of R¹². In certain embodiments, R⁶ is a 3-7 membered saturated monocyclic heterocyclyl containing 1 heteroatom independently selected from oxygen and nitrogen, wherein the monocyclic heterocyclyl is substituted with 0, 1, or 2 occurrences of R¹². In certain embodiments, R⁶ is a 3-7 membered saturated monocyclic heterocyclyl containing 2 heteroatoms independently selected from oxygen and nitrogen, wherein the monocyclic heterocyclyl is substituted with 0, 1, or 2 occurrences of R¹². In certain embodiments, R⁶ is a 3-7 membered partially unsaturated monocyclic heterocyclyl containing 1 or 2 heteroatoms independently selected from oxygen and nitrogen, wherein the monocyclic heterocyclyl is substituted with 0, 1, or 2 occurrences of R¹². In certain embodiments, R⁶ is a 3-7 membered partially unsaturated monocyclic heterocyclyl containing 1 heteroatom independently selected from oxygen and nitrogen, wherein the monocyclic heterocyclyl is substituted with 0, 1, or 2 occurrences of R¹². In certain embodiments, R⁶ is a 3-7 membered partially unsaturated monocyclic heterocyclyl containing 2 heteroatoms independently selected from oxygen and nitrogen, wherein the monocyclic heterocyclyl is substituted with 0, 1, or 2 occurrences of R¹². In certain embodiments, R⁶ is a 4 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹². In certain embodiments, R⁶ is a 5 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹². In certain embodiments, R⁶ is a 6 membered saturated or partially unsaturated monocyclic heterocyclyl containing 1 or 2 heteroatoms independently selected from oxygen and nitrogen, wherein the monocyclic heterocyclyl is substituted with 0, 1, or 2 occurrences of R¹². In certain embodiments, R⁶ is a 6-7 membered saturated bicyclic heterocyclyl containing 1 nitrogen atom, wherein the bicyclic heterocyclyl is substituted with 0, 1, or 2 occurrences of R¹². In certain embodiments, R⁶ is a 6 membered saturated bicyclic heterocyclyl containing 1 nitrogen atom, wherein the bicyclic heterocyclyl is substituted with 0, 1, or 2 occurrences of R¹². In certain embodiments, R⁶ is a 6 membered saturated bicyclic heterocyclyl containing 1 nitrogen atom. In certain embodiments, R⁶ is a 6 membered saturated bicyclic heterocyclyl containing 1 nitrogen atom, wherein the bicyclic heterocyclyl is substituted with 1 occurrence of R¹². In certain embodiments, R⁶ is a 6 membered saturated bicyclic heterocyclyl containing 1 nitrogen atom, wherein the bicyclic heterocyclyl is substituted with 2 occurrences of R¹². In certain embodiments, R⁶ is a 7 membered saturated bicyclic heterocyclyl containing 1 nitrogen atom, wherein the bicyclic heterocyclyl is substituted with 0, 1, or 2 occurrences of R¹². In certain embodiments, R⁶ is a 7 membered saturated bicyclic heterocyclyl containing 1 nitrogen atom. In certain embodiments, R⁶ is a 7 membered saturated bicyclic heterocyclyl containing 1 nitrogen atom, wherein the bicyclic heterocyclyl is substituted with 1 occurrence of R¹². In certain embodiments, R⁶ is a 7 membered saturated bicyclic heterocyclyl containing 1 nitrogen atom, wherein the bicyclic heterocyclyl is substituted with 2 occurrences of R¹². In certain embodiments, R⁶ is —C(CH3)₂OCH₃. In certain embodiments, R⁶ is —CH(CH₃)OCH₃. In certain embodiments, R⁶ is —CH₂OH. In certain embodiments, R⁶ is

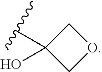 [SMILES: *C1(O)COC1]

In certain embodiments, R⁶ is

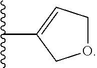 [SMILES: *C1=CCOC1]

In certain embodiments, R⁶ is

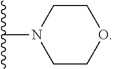 [SMILES: *N1CCOCC1]

In certain embodiments, R⁶ is

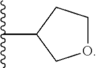 [SMILES: *C1CCOC1]

In certain embodiments, R⁶ is

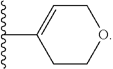 [SMILES: *C1=CCOCC1]

In certain embodiments, R⁶ is

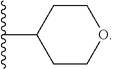 [SMILES: *C1CCOCC1]

In certain embodiments, R⁶ is

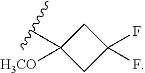 [SMILES: *C1(OC)CC(F)(F)C1]

In certain embodiments, R⁶ is

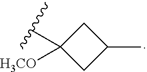 [SMILES: *C1(OC)CC(C)C1]

In certain embodiments, R⁶ is

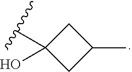 [SMILES: *C1(O)CC(C)C1]

In certain embodiments, R⁶ is

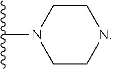

In certain embodiments, R⁶ is

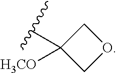 [SMILES: *C1(OC)COC1]

In certain embodiments, R⁶ is

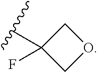 [SMILES: *C1(F)COC1]

In certain embodiments, R⁶ is selected from the groups depicted in the compounds in Table 1 below.

As defined generally above, R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 4 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 5 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, C₁₋₆ hydroxyalkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₁ alkyl. In certain embodiments, R⁸ is C₂ alkyl. In certain embodiments, R⁸ is C₃ alkyl. In certain embodiments, R⁸ is C₄ alkyl. In certain embodiments, R⁸ is C₅ alkyl. In certain embodiments, R⁸ is C₆ alkyl. In certain embodiments, R⁸ is C₁₋₆ hydroxyalkyl. In certain embodiments, R⁸ is C₁ hydroxyalkyl. In certain embodiments, R⁸ is C₂ hydroxyalkyl. In certain embodiments, R⁸ is C₃ hydroxyalkyl. In certain embodiments, R⁸ is C₄ hydroxyalkyl. In certain embodiments, R⁸ is C₅ hydroxyalkyl. In certain embodiments, R⁸ is C₆ hydroxyalkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₁ alkyl. In certain embodiments, R⁹ is C₂ alkyl. In certain embodiments, R⁹ is C₃ alkyl. In certain embodiments, R⁹ is C₄ alkyl. In certain embodiments, R⁹ is C₅ alkyl. In certain embodiments, R⁹ is C₆ alkyl. In certain embodiments, R⁹ is C₁₋₆ hydroxyalkyl. In certain embodiments, R⁹ is C₁ hydroxyalkyl. In certain embodiments, R⁹ is C₂ hydroxyalkyl. In certain embodiments, R⁹ is C₃ hydroxyalkyl. In certain embodiments, R⁹ is C₄ hydroxyalkyl. In certain embodiments, R⁹ is C₅ hydroxyalkyl. In certain embodiments, R⁹ is C₆ hydroxyalkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or (C₀₋₅ alkylene)-C₃₋₇ cycloalkyl. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is (C₀₋₅ alkylene)-C₃₋₇ cycloalkyl. In certain embodiments, R¹⁰ is —C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ represents independently for each occurrence halo. In certain embodiments, R¹¹ represents independently for each occurrence fluoro. In certain embodiments, R¹¹ represents independently for each occurrence hydroxyl. In certain embodiments, R¹¹ represents independently for each occurrence C₁₋₆ alkyl. In certain embodiments, R¹¹ represents independently for each occurrence C₁ alkyl. In certain embodiments, R¹¹ represents independently for each occurrence C₂ alkyl. In certain embodiments, R¹¹ represents independently for each occurrence C₃ alkyl. In certain embodiments, R¹¹ represents independently for each occurrence C₄ alkyl. In certain embodiments, R¹¹ represents independently for each occurrence C₅ alkyl. In certain embodiments, R¹¹ represents independently for each occurrence C₆ alkyl. In certain embodiments, R¹¹ represents independently for each occurrence C₁₋₆ haloalkyl. In certain embodiments, R¹¹ represents independently for each occurrence C₁ haloalkyl. In certain embodiments, R¹¹ represents independently for each occurrence C₂ haloalkyl. In certain embodiments, R¹¹ represents independently for each occurrence C₃ haloalkyl. In certain embodiments, R¹¹ represents independently for each occurrence C₄ haloalkyl. In certain embodiments, R¹¹ represents independently for each occurrence C₅ haloalkyl. In certain embodiments, R¹¹ represents independently for each occurrence C₆ haloalkyl. In certain embodiments, R¹¹ represents independently for each occurrence C₁₋₆ alkoxyl. In certain embodiments, R¹¹ represents independently for each occurrence C₁ alkoxyl. In certain embodiments, R¹¹ represents independently for each occurrence C₂ alkoxyl. In certain embodiments, R¹¹ represents independently for each occurrence C₃ alkoxyl. In certain embodiments, R¹¹ represents independently for each occurrence C₄ alkoxyl. In certain embodiments, R¹¹ represents independently for each occurrence C₅ alkoxyl. In certain embodiments, R¹¹ represents independently for each occurrence C₆ alkoxyl. In certain embodiments, R¹¹ represents independently for each occurrence C₃₋₇ cycloalkyl. In certain embodiments, R¹¹ is selected from the groups depicted in the compounds in Table 1 below.

As defined generally above, R¹² represents independently for each occurrence C₁₋₆ alkyl, C₁₋₆ alkoxyl, halo, hydroxyl, C₁₋₆ haloalkyl, oxo, cyano, or —C(O)—(C₁₋₄ alkyl); or two R¹² groups taken together with the carbon atom to which they are attached form a 3-7 membered saturated carbocyclic ring. In certain embodiments, R¹² represents independently for each occurrence C₁₋₆ alkyl. In certain embodiments, R¹² represents independently for each occurrence C₁ alkyl. In certain embodiments, R¹² represents independently for each occurrence C₂ alkyl. In certain embodiments, R¹² represents independently for each occurrence C₃ alkyl. In certain embodiments, R¹² represents independently for each occurrence C₄ alkyl. In certain embodiments, R¹² represents independently for each occurrence C₅ alkyl. In certain embodiments, R¹² represents independently for each occurrence C₆ alkyl. In certain embodiments, R¹² represents independently for each occurrence C₁₋₆ alkoxyl. In certain embodiments, R¹² represents independently for each occurrence C₁ alkoxyl. In certain embodiments, R¹² represents independently for each occurrence C₂ alkoxyl. In certain embodiments, R¹² represents independently for each occurrence C₃ alkoxyl. In certain embodiments, R¹² represents independently for each occurrence C₄ alkoxyl. In certain embodiments, R¹² represents independently for each occurrence C₅ alkoxyl. In certain embodiments, R¹² represents independently for each occurrence C₆ alkoxyl. In certain embodiments, R¹² represents independently for each occurrence halo. In certain embodiments, R¹² represents independently for each occurrence fluoro. In certain embodiments, R¹² represents independently for each occurrence hydroxyl. In certain embodiments, R¹² represents independently for each occurrence C₁₋₆ haloalkyl. In certain embodiments, R¹² represents independently for each occurrence C₁ haloalkyl. In certain embodiments, R¹² represents independently for each occurrence C₂ haloalkyl. In certain embodiments, R¹² represents independently for each occurrence C₃ haloalkyl. In certain embodiments, R¹² represents independently for each occurrence C₄ haloalkyl. In certain embodiments, R¹² represents independently for each occurrence C₅ haloalkyl. In certain embodiments, R¹² represents independently for each occurrence C₆ haloalkyl. In certain embodiments, R¹² represents independently for each occurrence oxo. In certain embodiments, R¹² represents independently for each occurrence cyano. In certain embodiments, R¹² represents independently for each occurrence —C(O)—(C₁₋₄ alkyl). In certain embodiments, two R¹² groups taken together with the carbon atom to which they are attached form a 3-7 membered saturated carbocyclic ring. In certain embodiments, two R¹² groups taken together with the carbon atom to which they are attached form a 3 membered saturated carbocyclic ring. In certain embodiments, two R¹² groups taken together with the carbon atom to which they are attached form a 4 membered saturated carbocyclic ring. In certain embodiments, two R¹² groups taken together with the carbon atom to which they are attached form a 5 membered saturated carbocyclic ring. In certain embodiments, two R¹² groups taken together with the carbon atom to which they are attached form a 6 membered saturated carbocyclic ring. In certain embodiments, two R¹² groups taken together with the carbon atom to which they are attached form a 7 membered saturated carbocyclic ring. In certain embodiments, R¹² is selected from the groups depicted in the compounds in Table 1 below.

As defined generally above, R¹³ and R¹⁴ are independently hydrogen or C₁₋₄ alkyl. In certain embodiments, R¹³ is hydrogen and R¹⁴ is C₁₋₄ alkyl. In certain embodiments, R¹³ is hydrogen and R¹⁴ is methyl. In certain embodiments, each of R¹³ and R¹⁴ is hydrogen. In certain embodiments, R¹³ is hydrogen. In certain embodiments, R¹³ is C₁₋₄ alkyl. In certain embodiments, R¹³ is C₁ alkyl. In certain embodiments, R¹³ is C₂ alkyl. In certain embodiments, R¹³ is C₃ alkyl. In certain embodiments, R¹³ is C₄ alkyl. In certain embodiments, R¹⁴ is hydrogen. In certain embodiments, R¹⁴ is C₁₋₄ alkyl. In certain embodiments, R¹⁴ is C₁ alkyl. In certain embodiments, R¹⁴ is C₂ alkyl. In certain embodiments, R¹⁴ is C₃ alkyl. In certain embodiments, R¹⁴ is C₄ alkyl. In certain embodiments, R¹³ is selected from the groups depicted in the compounds in Table 1 below. In certain embodiments, R¹⁴ is selected from the groups depicted in the compounds in Table 1 below.

As defined generally above, n, m, x, and y are independently 0, 1, or 2. In certain embodiments, n is 0. In certain embodiments, n is 1. In certain embodiments, n is 2. In certain embodiments, m is 0. In certain embodiments, m is 1. In certain embodiments, m is 2. In certain embodiments, x is 0. In certain embodiments, x is 1. In certain embodiments, x is 2. In certain embodiments, y is 0. In certain embodiments, y is 1. In certain embodiments, y is 2.

As defined generally above, q is 0, 1, 2, or 3. In certain embodiments q is 0. In certain embodiments q is 1. In certain embodiments q is 2. In certain embodiments q is 3.

The description above describes multiple embodiments relating to compounds of Formula I. The patent application specifically contemplates all combinations of the embodiments.

Another aspect of the invention provides a compound represented by Formula I-1

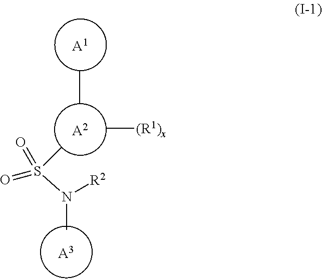

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is pyrazolylene or 1,2,3-triazolylene;
A³ is

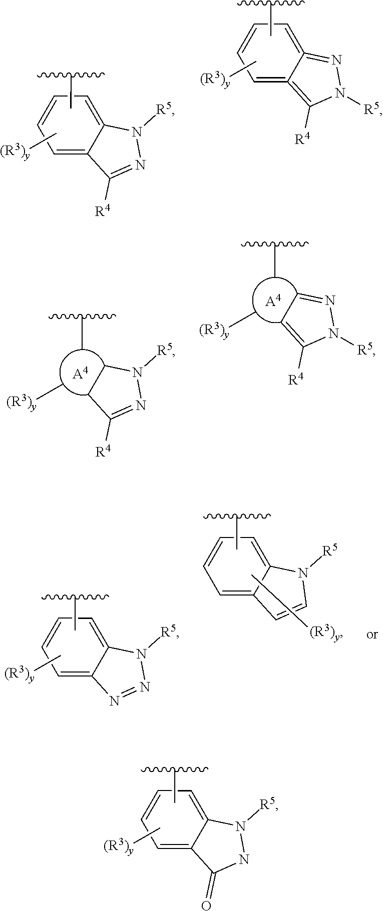

A⁴ is a 6-membered aromatic ring containing 1 nitrogen atom;
R¹ represents independently for each occurrence halo, C₁₋₄ alkyl, C₁₋₄ halolkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R′)S(O₂)R¹⁰;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

The definitions of variables in Formula I-1 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-1.

As defined generally above, A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶. In certain embodiments, A¹ is a 6-membered heteroaryl containing 1, 2, or 3 heteratoms independently selected from oxygen, nitrogen, and sulfur, wherein the heteroaryl is substituted with n occurrences of R⁶. In certain embodiments, A¹ is pyridinyl, pyrimidinyl, pyridazinyl, or pyrazinyl, each of which is substituted with n occurrences of R⁶. In certain embodiments, A¹ is pyridinyl substituted with n occurrences of R⁶. In certain embodiments, A¹ is

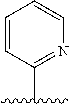 [SMILES: *c1ccccn1]

substituted with n occurrences of R⁶. In certain embodiments, A¹ is

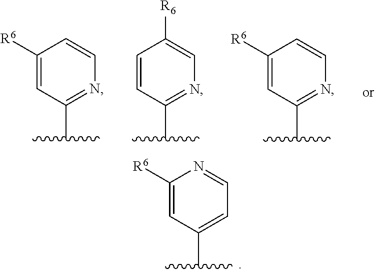

In certain embodiments, A¹ is

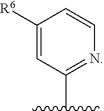

In certain embodiments, A¹ is

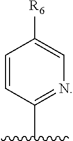

In certain embodiments, A¹ is

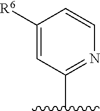

In certain embodiments, A¹ is

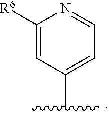

In certain embodiments, A¹ is is

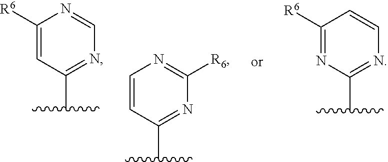

In certain embodiments, A¹ is

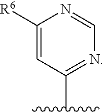

In certain embodiments, A¹ is

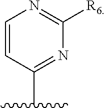

In certain embodiments, A¹ is

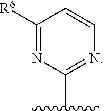

In certain embodiments, A¹ is a 5-6 membered heteroaryl containing 1, 2, or 3 heteratoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl are substituted with n occurrences of R⁶. In certain embodiments, A¹ is a 5-membered heteroaryl containing 1, 2, or 3 heteratoms independently selected from oxygen, nitrogen, and sulfur, wherein the heteroaryl is substituted with n occurrences of R⁶. In certain embodiments, A¹ is a 9-10 membered bicyclic heteroaryl containing 1, 2, or 3 heteroatoms independently selected from oxygen, nitrogen, and sulfur. In certain embodiments, A¹ is a 9-10 membered bicyclic heteroaryl containing 1 heteroatom selected from oxygen, nitrogen, and sulfur. In certain embodiments, A¹ is a 9-10 membered bicyclic heteroaryl containing 2 heteroatoms independently selected from oxygen, nitrogen, and sulfur. In certain embodiments, A¹ is a 9-10 membered bicyclic heteroaryl containing 3 heteroatoms independently selected from oxygen, nitrogen, and sulfur. In certain embodiments, A¹ is a 3-10 membered saturated heterocyclyl containing 1, 2, or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom independently selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶. In certain embodiments, A¹ is selected from the groups depicted in the compounds in Table 1 below.

As defined generally above, A² is pyrazolylene or 1,2,3-triazolylene. In certain embodiments, A² is pyrazolylene. In certain embodiments, A² is 1,2,3-triazolylene.

In certain embodiments, A² is

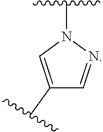 [SMILES: *c1cnn(*)c1]

In certain embodiments, A² is

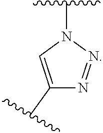 [SMILES: *c1cn(*)nn1]

In certain embodiments, A² is selected from the groups depicted in the compounds in Table 1 below.

As defined generally above, A³ is

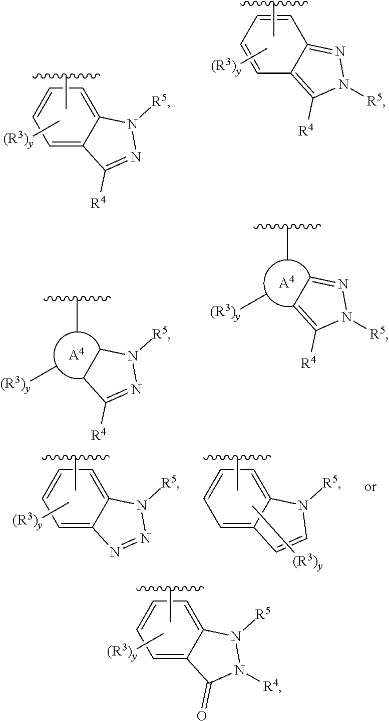

In certain embodiments, A³ is

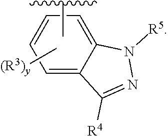

In certain embodiments, A³ is

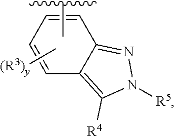

In certain embodiments, A³ is

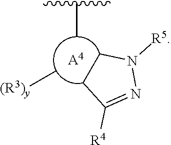

In certain embodiments, A³ is

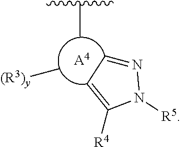

In certain embodiments, A³ is

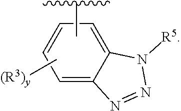

In certain embodiments, A³ is

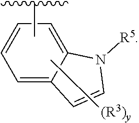

In certain embodiments, A³ is

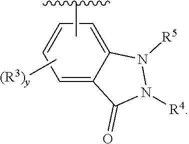

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R⁵ is C₁₋₄ alkyl. In certain embodiments, R⁵ is methyl. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl In certain embodiments, R³ is C₁₋₆ haloalkyl. In certain embodiments, R³ is C₁₋₆ deuteroalkyl. In certain embodiments, R³ is C₁₋₆ deuteroalkoxyl. In certain embodiments, R³ is C₃₋₇ halocycloalkyl. In certain embodiments, R³ is C₃₋₇ hydroxycycloalkyl. In certain embodiments, R³ is —O—C₃₋₇ cycloalkyl. In certain embodiments, R³ is —(C₀₋₄ alkylene)-CN. In certain embodiments, R³ is cyano. In certain embodiments, R³ is C₂₋₄ alkynyl. In certain embodiments, R³ is —N(R⁸)(R⁹). In certain embodiments, R³ is CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, R³ is a 3-7 membered saturated heterocyclyl containing 1 or 2 heteroatoms independently selected from oxygen and nitrogen, wherein the heterocyclyl is substituted with 0 or 1 halo. In certain embodiments, R³ is a 3-7 membered saturated heterocyclyl containing 1 heteroatom selected from oxygen and nitrogen, wherein the heterocyclyl is substituted with 0 or 1 halo. In certain embodiments, R³ is a 3-7 membered saturated heterocyclyl containing 2 heteroatoms independently selected from oxygen and nitrogen, wherein the heterocyclyl is substituted with 0 or 1 halo. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is F. In certain embodiments, R⁴ is Cl. In certain embodiments, R⁴ is Br. In certain embodiments, R⁴ is I. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R⁸)S(O₂)R¹⁰. In certain embodiments, R⁶ is C₁₋₆ haloalkyl. In certain embodiments, R⁶ is —CF₃. In certain embodiments, R⁶ is C₁₋₆ alkyl. In certain embodiments, R⁶ is methyl. In certain embodiments, R⁶ is C₃₋₇ cycloalkyl. In certain embodiments, R⁶ is cyclopropyl. In certain embodiments, R⁶ is halo. In certain embodiments, R⁶ is hydroxyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —N(R⁸)(R⁹). In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R′)S(O₂)R¹⁰. In certain embodiments, R⁶ is selected from the groups depicted in the compounds in Table 1 below.

As defined generally above, R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 4 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 5 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or (C₀₋₅ alkylene)-C₃₋₇ cycloalkyl. In certain embodiments, R¹⁰ is C₁₋₆ alkyl. In certain embodiments, R¹⁰ is (C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ represents independently for each occurrence halo. In certain embodiments, R¹¹ represents independently for each occurrence fluoro. In certain embodiments, R¹¹ represents independently for each occurrence hydroxyl. In certain embodiments, R¹¹ represents independently for each occurrence C₁₋₆ alkyl. In certain embodiments, R¹¹ represents independently for each occurrence C₁ alkyl. In certain embodiments, R¹¹ represents independently for each occurrence C₂ alkyl. In certain embodiments, R¹¹ represents independently for each occurrence C₃ alkyl. In certain embodiments, R¹¹ represents independently for each occurrence C₄ alkyl. In certain embodiments, R¹¹ represents independently for each occurrence C₅ alkyl. In certain embodiments, R¹¹ represents independently for each occurrence C₆ alkyl. In certain embodiments, R¹¹ represents independently for each occurrence C₁₋₆ haloalkyl. In certain embodiments, R¹¹ represents independently for each occurrence C₁ haloalkyl. In certain embodiments, R¹¹ represents independently for each occurrence C₂ haloalkyl. In certain embodiments, R¹¹ represents independently for each occurrence C₃ haloalkyl. In certain embodiments, R¹¹ represents independently for each occurrence C₄ haloalkyl. In certain embodiments, R¹¹ represents independently for each occurrence C₅ haloalkyl. In certain embodiments, R¹¹ represents independently for each occurrence C₆ haloalkyl. In certain embodiments, R¹¹ represents independently for each occurrence C₁₋₆ alkoxyl. In certain embodiments, R¹¹ represents independently for each occurrence C₁ alkoxyl. In certain embodiments, R¹¹ represents independently for each occurrence C₂ alkoxyl. In certain embodiments, R¹¹ represents independently for each occurrence C₃ alkoxyl. In certain embodiments, R¹¹ represents independently for each occurrence C₄ alkoxyl. In certain embodiments, R¹¹ represents independently for each occurrence C₅ alkoxyl. In certain embodiments, R¹¹ represents independently for each occurrence C₆ alkoxyl. In certain embodiments, R¹¹ represents independently for each occurrence C₃₋₇ cycloalkyl. In certain embodiments, R¹¹ is selected from the groups depicted in the compoun”s in 'Table 1 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 1 below.

As defined generally above, n, m, and x are independently 0, 1, or 2. In certain embodiments, x is 0. In certain embodiments, n is 1. In certain embodiments, n is 2. In certain embodiments, n is 0. In certain embodiments, x is 1. In certain embodiments, x is 2. In certain embodiments, m is 0. In certain embodiments, m is 1. In certain embodiments, m is 2. In certain embodiments, n is selected from the corresponding value in the groups depicted in the compounds in Table 1 below. In certain embodiments, m is selected from the corresponding value in the groups depicted in the compounds in Table 1 below. In certain embodiments, x is selected from the corresponding value in the groups depicted in the compounds in Table 1 below.

The description above describes multiple embodiments relating to compounds of Formula I-1. The patent application specifically contemplates all combinations of the embodiments.

In certain embodiments, the compound of Formula I is further defined by Formula Ia:

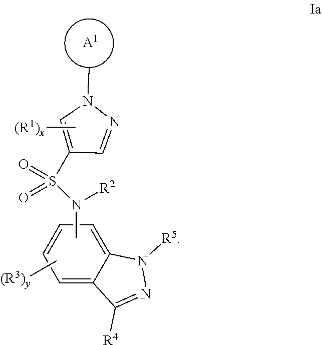

or a pharmaceutically acceptable salt thereof. In certain embodiments, the definition of variables R¹, R², R³, R⁴, R⁵, A¹, x, and y is one of the embodiments described above in connection with Formula I.

The description above describes multiple embodiments relating to compounds of Formula Ia. The patent application specifically contemplates all combinations of the embodiments.

In certain embodiments, the compound of Formula I is further defined by Formula Ib or Ic:

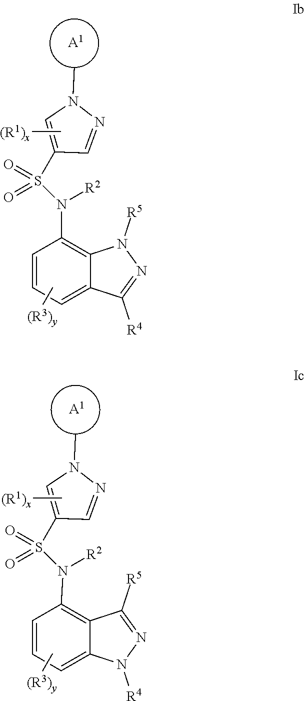

or a pharmaceutically acceptable salt thereof. In certain embodiments, the definition of variables R¹, R², R³, R⁴, R⁵, A¹, x, and y is one of the embodiments described above in connection with Formula I. In certain embodiments, the compound of Formula I is further defined by Formula Ib or a pharmaceutically acceptable salt thereof. In certain embodiments, the compound of Formula I is further defined by Formula Ic or a pharmaceutically acceptable salt thereof.

The description above describes multiple embodiments relating to compounds of Formulae Ib and Ic. The patent application specifically contemplates all combinations of the embodiments.

In certain embodiments, the compound of Formula I is further defined by Formula Id or Ie:

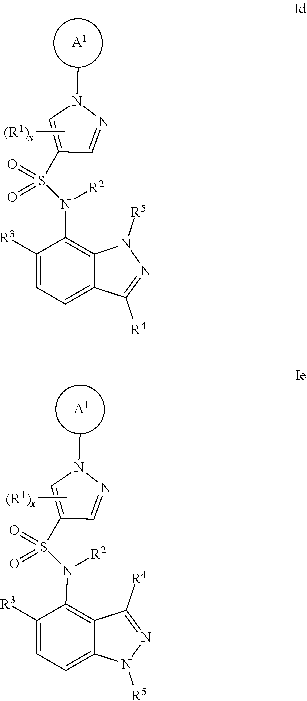

or a pharmaceutically acceptable salt thereof. In certain embodiments, the definition of variables R¹, R², R³, R⁴, R⁵, A¹, and x is one of the embodiments described above in connection with Formula I. In certain embodiments, the compound of Formula I is further defined by Formula Id or a pharmaceutically acceptable salt thereof. In certain embodiments, the compound of Formula I is further defined by Formula Ie or a pharmaceutically acceptable salt thereof.

The description above describes multiple embodiments relating to compounds of Formulae Id and Ie. The patent application specifically contemplates all combinations of the embodiments.

In certain embodiments, the compound of Formula I is further defined by Formula If:

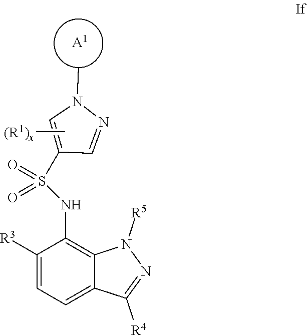

or a pharmaceutically acceptable salt thereof. In certain embodiments, the definition of variables R¹, R³, R⁴, R⁵, A¹, and x is one of the embodiments described above in connection with Formula I.

The description above describes multiple embodiments relating to compounds of Formula If. The patent application specifically contemplates all combinations of the embodiments.

In certain embodiments, the compound of Formula I is further defined by Formula Ig or Ih:

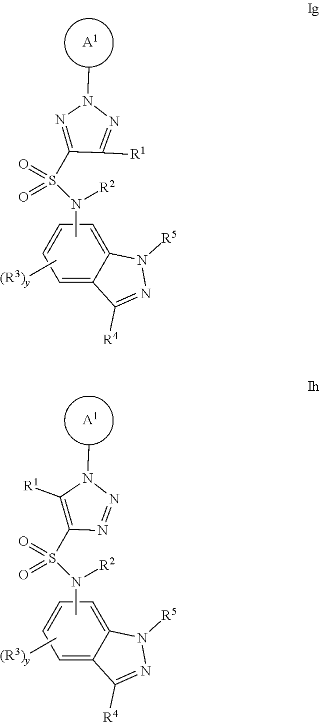

Or a pharmaceutically acceptable salt thereof. In certain embodiments, the definition of variables R¹, R², R³, R⁴, R⁵, A¹, and y is one of the embodiments described above in connection with Formula I. In certain embodiments, the compound of Formula I is further defined by Formula Ig or a pharmaceutically acceptable salt thereof. In certain embodiments, the compound of Formula I is further defined by Formula Ih or a pharmaceutically acceptable salt thereof.

The description above describes multiple embodiments relating to compounds of Formulae Ig and Ih. The patent application specifically contemplates all combinations of the embodiments.

In certain embodiments, the compound of Formula I is further defined by Formula Ii, Ij, Ik, or Il:

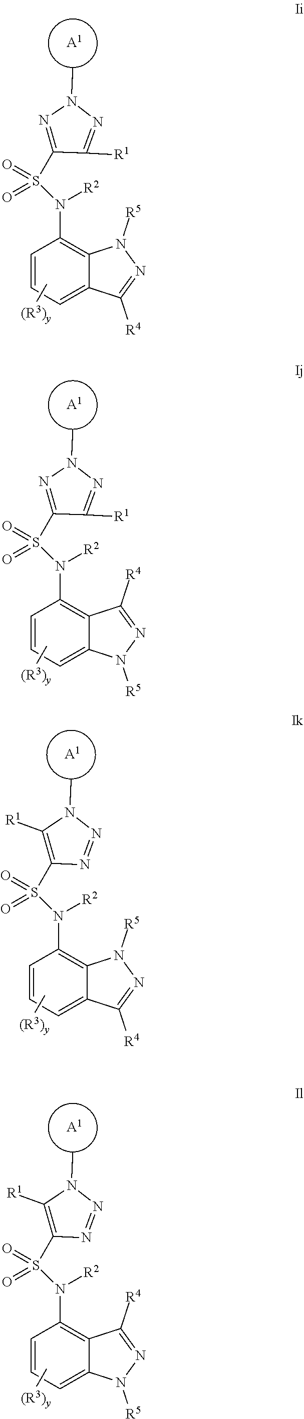

or a pharmaceutically acceptable salt thereof. In certain embodiments, the definition of variables R¹, R², R³, R⁴, R⁵, A¹, and y is one of the embodiments described above in connection with Formula I. In certain embodiments, the compound of Formula I is further defined by Formula Ii or a pharmaceutically acceptable salt thereof. In certain embodiments, the compound of Formula I is further defined by Formula Ij or a pharmaceutically acceptable salt thereof. In certain embodiments, the compound of Formula I is further defined by Formula Ik or a pharmaceutically acceptable salt thereof. In certain embodiments, the compound of Formula I is further defined by Formula Il or a pharmaceutically acceptable salt thereof.

The description above describes multiple embodiments relating to compounds of Formulae Ii, Ij, Ik, and Il. The patent application specifically contemplates all combinations of the embodiments.

In certain embodiments, the compound of Formula I is further defined by Formula Im, In, Io, or Ip:

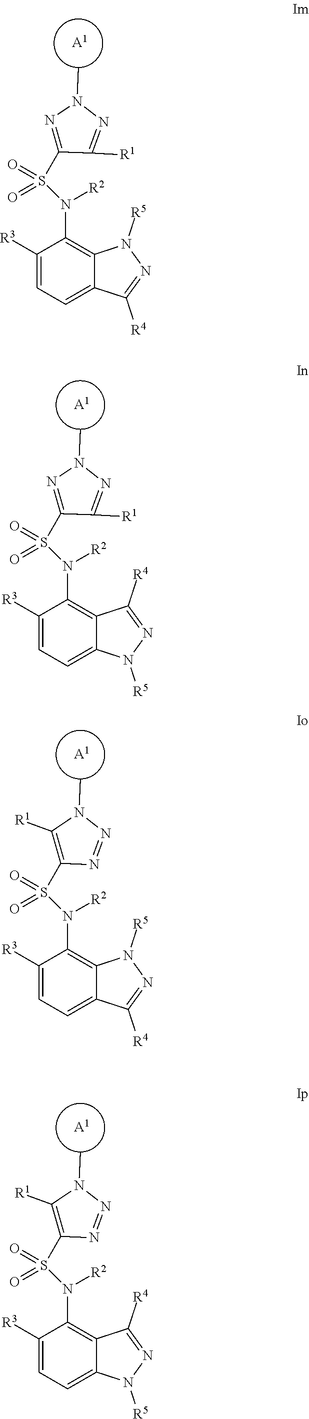

or a pharmaceutically acceptable salt thereof. In certain embodiments, the definition of variables R¹, R³, R⁴, R⁵, and A¹ is one of the embodiments described above in connection with Formula I. In certain embodiments, the compound of Formula I is further defined by Formula Im or a pharmaceutically acceptable salt thereof. In certain embodiments, the compound of Formula I is further defined by Formula In or a pharmaceutically acceptable salt thereof. In certain embodiments, the compound of Formula I is further defined by Formula Io or a pharmaceutically acceptable salt thereof. In certain embodiments, the compound of Formula I is further defined by Formula Ip or a pharmaceutically acceptable salt thereof.

The description above describes multiple embodiments relating to compounds of Formulae Im, In, Io, and Ip. The patent application specifically contemplates all combinations of the embodiments.

In certain embodiments, the compound of Formula I is further defined by Formula Iq or a pharmaceutically acceptable salt thereof.

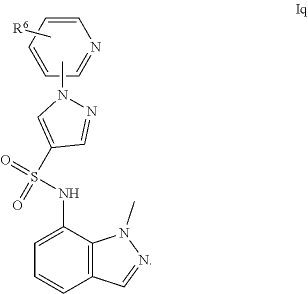

In certain embodiments, the compound of Formula I is further defined by Formula Ir or a pharmaceutically acceptable salt thereof:

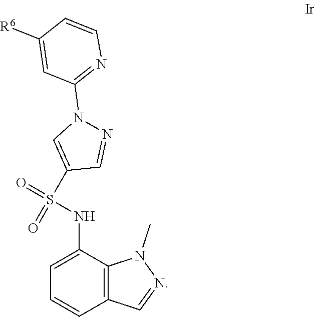

In certain embodiments, R⁶ is C₁₋₆ haloalkyl, —C₁₋₆ hydroxyalkyl, —(C₁₋₄ alkylene)-CN, or a 3-7 membered saturated or partially unsaturated monocyclic heterocyclyl containing 1 or 2 heteroatoms independently selected from oxygen and nitrogen, wherein the monocyclic heterocyclyl is substituted with 1 occurrence of R¹². In certain embodiments, R⁶ is C₁₋₃ haloalkyl. The compound of claim 1, wherein R⁶ is —C₂₋₆ hydroxyalkyl. In certain embodiments, R⁶ is —(C₂₋₄ alkylene)-CN. In certain embodiments, R⁶ is a 3-7 membered saturated or partially unsaturated monocyclic heterocyclyl containing 1 or 2 heteroatoms independently selected from oxygen and nitrogen, wherein the monocyclic heterocyclyl is substituted with 1 occurrence of R¹². In certain embodiments, R⁶ is a tetrahydrofuranyl substituted by hydroxyl.

Another aspect of the invention provides a compound represented by Formula 1-2

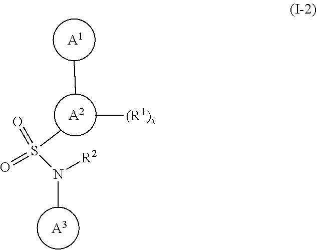

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2, or 3 heteratoms independently selected from oxygen, nitrogen, and sulfur, or a 5-6 membered partially unsaturated heterocyclyl containing 1 nitrogen atom, wherein the heteroaryl and saturated heterocyclyl are substituted with n occurrences of R⁶, and wherein the partially unstaturated heterocyclyl is substituted with n occurrences of R⁶ and 1 occurrence of oxo;
A² is pyrazolylene or 1,2,3-triazolylene;

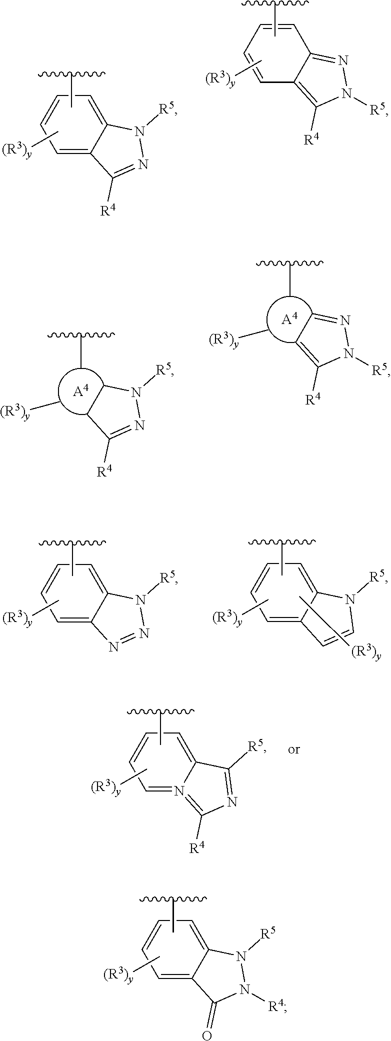

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, C₁₋₄ haloalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), C₁₋₆ hydroxyalkyl, —C(O)R¹⁰, —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, C₁₋₄ alkoxyl, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with q occurrences of R¹², —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R′)S(O₂)R¹⁰, —C₁₋₆ hydroxyalkyl, —(C₁₋₄ alkylene)-(C₁₋₆ alkoxyl), or a 3-7 membered saturated or partially unsaturated heterocyclyl containing 1 or 2 heteroatoms independently selected from oxygen and nitrogen, wherein the heterocyclyl is substituted with 0 or 1 occurrences of R¹²;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
R¹² represents independently for each occurrence C₁₋₆ alkyl, C₁₋₆ alkoxyl, halo, or hydroxyl;
n, m, x, and y are independently 0, 1, or 2; and
q is 0, 1, 2, or 3.

The definitions of variables in Formula I-2 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).′

In certain embodiments, the compound is a compound of Formula I-2.

As defined generally above, A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2, or 3 heteratoms independently selected from oxygen, nitrogen, and sulfur, or a 5-6 membered partially unsaturated heterocyclyl containing 1 nitrogen atom, wherein the heteroaryl and saturated heterocyclyl are substituted with n occurrences of R⁶, and wherein the partially unstaturated heterocyclyl is substituted with n occurrences of R⁶ and 1 occurrence of oxo. In certain embodiments, A¹ is a 5-6 membered heteroaryl containing 1, 2, or 3 heteratoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl are substituted with n occurrences of R⁶. In certain embodiments, A¹ is a 5-6 membered heteroaryl containing 1, 2, or 3 heteratoms independently selected from oxygen, nitrogen, and sulfur, wherein the heteroaryl is substituted with n occurrences of R⁶. In certain embodiments, A¹ is a 6-membered heteroaryl containing 1, 2, or 3 heteratoms independently selected from oxygen, nitrogen, and sulfur, wherein the heteroaryl is substituted with n occurrences of R⁶. In certain embodiments, A¹ is pyridinyl, pyrimidinyl, pyridazinyl, or pyrazinyl, each of which is substituted with n occurrences of R⁶. In certain embodiments, A¹ is pyridinyl substituted with n occurrences of R⁶. In certain embodiments, A¹ is

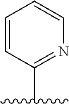 [SMILES: *c1ccccn1]

substituted with n occurrences of R⁶. In certain embodiments, A¹ is

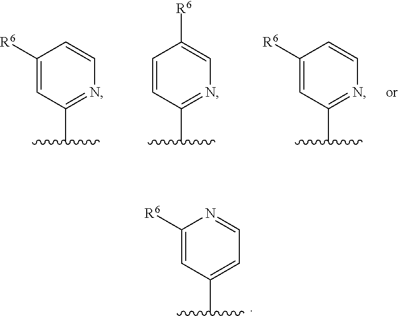

In certain embodiments, A¹ is

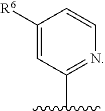

In certain embodiments, A¹ is

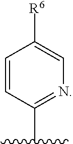

In certain embodiments, A¹ is

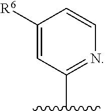

In certain embodiments, A¹ is

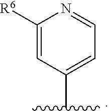

In certain embodiments, A¹ is is

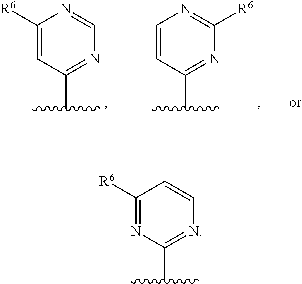

In certain embodiments, A¹ is

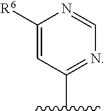

In certain embodiments, A¹ is

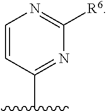

In certain embodiments, A¹ is

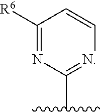

In certain embodiments, A¹ is a 5-membered heteroaryl containing 1, 2, or 3 heteratoms independently selected from oxygen, nitrogen, and sulfur, wherein the heteroaryl is substituted with n occurrences of R⁶. In certain embodiments, A¹ is a 9-10 membered bicyclic heteroaryl containing 1, 2, or 3 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 1 heteroatom selected from oxygen, nitrogen, and sulfur, wherein the heteroaryl is substituted with n occurrences of R⁶. In certain embodiments, A¹ is a 9-10 membered bicyclic heteroaryl containing 2 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 3 heteroatoms independently selected from oxygen, nitrogen, and sulfur, wherein the heteroaryl is substituted with n occurrences of R⁶. In certain embodiments, A¹ is a 3-10 membered saturated heterocyclyl containing 1, 2, or 3 heteroatoms independently selected from oxygen, nitrogen, and sulfur, wherein the saturated heterocyclyl is substituted with n occurrences of R⁶. In certain embodiments, A¹ is a 3-10 membered saturated heterocyclyl containing 1 heteroatom independently selected from oxygen, nitrogen, and sulfur, wherein the saturated heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the saturated heterocyclyl is substituted with n occurrences of R. In certain embodiments, A¹ is a 3-10 membered saturated heterocyclyl containing 3 heteroatoms independently selected from oxygen, nitrogen, and sulfur, wherein the saturated heterocyclyl is substituted with n occurrences of R⁶. In certain embodiments, A¹ is a 5-6 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is a 5 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is a 6 membered partially unsaturated heterocyclyl containing 1 nitrogen atom, wherein the partially unstaturated heterocyclyl is substituted with n occurrences of R⁶ and 1 occurrence of oxo. In certain embodiments, A¹ is selected from the groups depicted in the compounds in Table 1 below.

As defined generally above, A² is pyrazolylene or 1,2,3-triazolylene. In certain embodiments, A² is pyrazolylene. In certain embodiments, A² is 1,2,3-triazolylene. In certain embodiments, A² is

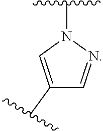 [SMILES: *c1cnn(*)c1]

In certain embodiments, A² is

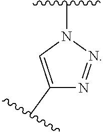 [SMILES: *c1cn(*)nn1]

In certain embodiments, A² is selected from the groups depicted in the compounds in Table 1 below.

As defined generally above, A³ is

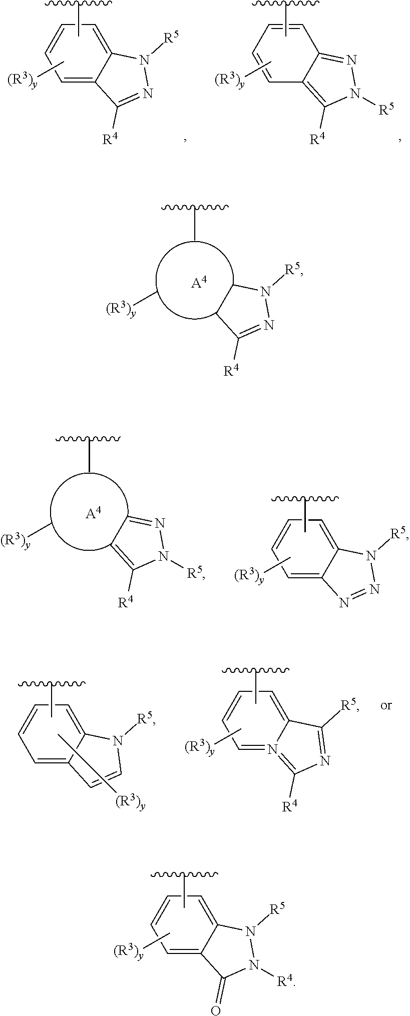

In certain embodiments, A³ is

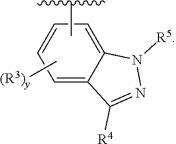

In certain embodiments, A³ is

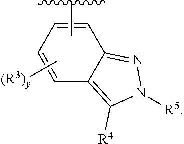

In certain embodiments, A³ is

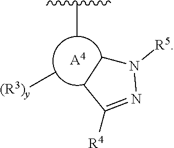

In certain embodiments, A³ is

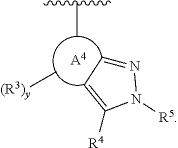

In certain embodiments, A³ is

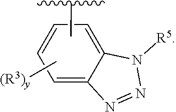

In certain embodiments, A³ is

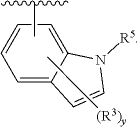

In certain embodiments, A³ is

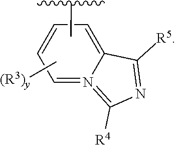

In certain embodiments, A³ is

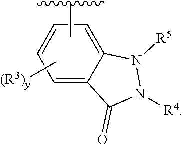

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, C₁₋₄ haloalkyl, —(C₁₋₄ alkylene)-(C₁₋₆ alkoxyl), or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ haloalkyl. In certain embodiments, R⁵ is C₁ haloalkyl. In certain embodiments, R⁵ is —CHF₂. In certain embodiments, R⁵ is C₂ haloalkyl. In certain embodiments, R⁵ is C₃ haloalkyl. In certain embodiments, R⁵ is C₄ haloalkyl. In certain embodiments, R⁵ is —(C₁₋₄ alkylene)-(C₁₋₆ alkoxyl). In certain embodiments, R⁵ is —CH₂CH₂OCH₃. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), C₁₋₆ hydroxyalkyl, —C(O)R¹⁰, —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R³ is C₁₋₆ alkyl. In certain embodiments, R³ is methyl. In certain embodiments, R³ is ethyl. In certain embodiments, R³ is C₃ alkyl. In certain embodiments, R³ is C₄ alkyl. In certain embodiments, R³ is C₅ alkyl. In certain embodiments, R³ is C₆ alkyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is C₂ alkoxyl. In certain embodiments, R³ is C₃ alkoxyl. In certain embodiments, R³ is C₄ alkoxyl. In certain embodiments, R³ is C₅ alkoxyl. In certain embodiments, R³ is C₆ alkoxyl. In certain embodiments, R³ is halo. In certain embodiments, R³ is hydroxyl In certain embodiments, R³ is C₁₋₆ haloalkyl. In certain embodiments, R³ is C₁ haloalkyl. In certain embodiments, R³ is C₂ haloalkyl. In certain embodiments, R³ is C₃ haloalkyl. In certain embodiments, R³ is C₄ haloalkyl. In certain embodiments, R³ is C₅ haloalkyl. In certain embodiments, R³ is C₆ haloalkyl. In certain embodiments, R³ is C₁₋₆ deuteroalkyl. In certain embodiments, R³ is C₁₋₆ deuteroalkoxyl. In certain embodiments, R³ is C₃₋₇ halocycloalkyl. In certain embodiments, R³ is C₃₋₇ hydroxycycloalkyl. In certain embodiments, R³ is —O—C₃₋₇ cycloalkyl. In certain embodiments, R³ is —(C₀₋₄ alkylene)-CN. In certain embodiments, R³ is cyano. In certain embodiments, R³ is C₂₋₄ alkynyl. In certain embodiments, R³ is —N(R⁸)(R⁹). In certain embodiments, R³ is C₁₋₆ hydroxyalkyl. In certain embodiments, R³ is C₁ hydroxyalkyl. In certain embodiments, R³ is C₂ hydroxyalkyl. In certain embodiments, R³ is —CH(CH₃)OH. In certain embodiments, R³ is C₃ hydroxyalkyl. In certain embodiments, R³ is C₄ hydroxyalkyl. In certain embodiments, R³ is C₅ hydroxyalkyl. In certain embodiments, R³ is C₆ hydroxyalkyl. In certain embodiments, R³ is —C(O)R¹⁰. In certain embodiments, R³ is —C(O)CH₃. In certain embodiments, R³ is CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, R³ is a 3-7 membered saturated heterocyclyl containing 1 or 2 heteroatoms independently selected from oxygen and nitrogen, wherein the heterocyclyl is substituted with 0 or 1 halo. In certain embodiments, R³ is a 3-7 membered saturated heterocyclyl containing 1 heteroatom selected from oxygen and nitrogen, wherein the heterocyclyl is substituted with 0 or 1 halo. In certain embodiments, R³ is a 3-7 membered saturated heterocyclyl containing 2 heteroatoms independently selected from oxygen and nitrogen, wherein the heterocyclyl is substituted with 0 or 1 halo. In certain embodiments, R³ is selected from the groups depicted in the compounds in Table 1 below.

As defined generally above, R⁴ is hydrogen, halo, C₁₋₄ alkoxyl, or C₁₋₄ alkyl. In certain embodiments, R⁴ is hydrogen. In certain embodiments, R⁴ is halo. In certain embodiments, R⁴ is F. In certain embodiments, R⁴ is Cl. In certain embodiments, R⁴ is Br. In certain embodiments, R⁴ is I. In certain embodiments, R⁴ is C₁₋₄ alkoxyl. In certain embodiments, R⁴ is C₁ alkoxyl. In certain embodiments, R⁴ is C₂ alkoxyl. In certain embodiments, R⁴ is C₃ alkoxyl. In certain embodiments, R⁴ is C₄ alkoxyl. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with q occurrences of R¹², —O—C₃₋₇ cycloalkyl, —N(R⁸)(R⁹), —C(O)R⁷, —C(O)N(R⁸)(R⁹), —N(R⁸)C(O)R¹⁰, —S(02)R¹⁰, —S(O₂)N(R⁸)(R⁹), —N(R′)S(O2)R¹⁰, —C₁₋₆ hydroxyalkyl, —(C₁₋₄ alkylene)-(C₁₋₆ alkoxyl), or a 3-7 membered saturated or partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is C₁₋₆ haloalkyl. In certain embodiments, R⁶ is —CF₃. In certain embodiments, R⁶ is C₁ haloalkyl. In certain embodiments, R⁶ is C₂ haloalkyl. In certain embodiments, R⁶ is C₃ haloalkyl. In certain embodiments, R⁶ is C₄ haloalkyl. In certain embodiments, R⁶ is C₅ haloalkyl. In certain embodiments, R⁶ is C₆ haloalkyl. In certain embodiments, R⁶ is C₁₋₆ alkyl. In certain embodiments, R⁶ is methyl. In certain embodiments, R⁶ is C₂ alkyl. In certain embodiments, R⁶ is C₃ alkyl. In certain embodiments, R⁶ is C₄ alkyl. In certain embodiments, R⁶ is C₅ alkyl. In certain embodiments, R⁶ is C₆ alkyl. In certain embodiments, R⁶ is C₃₋₇ cycloalkyl substituted with q occurrences of R¹². In certain embodiments, R⁶ is cycloalkyl. In certain embodiments, R⁶ is cyclopropyl. In certain embodiments, R⁶ is halo. In certain embodiments, R⁶ is hydroxyl. In certain embodiments, R⁶ is cyano. In certain embodiments, R⁶ is C₁₋₆ alkoxyl. In certain embodiments, R⁶ is C₁ alkoxyl. In certain embodiments, R⁶ is C₂ alkoxyl. In certain embodiments, R⁶ is C₃ alkoxyl. In certain embodiments, R⁶ is C₄ alkoxyl. In certain embodiments, R⁶ is C₅ alkoxyl. In certain embodiments, R⁶ is C₆ alkoxyl. In certain embodiments, R⁶ is —O—C₃₋₇ cycloalkyl. In certain embodiments, R⁶ is —N(R⁸)(R⁹). In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R′)S(O₂)R¹⁰. In certain embodiments, R⁶ is —C₁₋₆ hydroxyalkyl. In certain embodiments, R⁶ is —C₁ hydroxyalkyl. In certain embodiments, R⁶ is —C₂ hydroxyalkyl. In certain embodiments, R⁶ is —C₃ hydroxyalkyl. In certain embodiments, R⁶ is —C₄ hydroxyalkyl. In certain embodiments, R⁶ is —C₅ hydroxyalkyl. In certain embodiments, R⁶ is —C₆ hydroxyalkyl. In certain embodiments, R⁶ is —(C₁₋₄ alkylene)-(C₁₋₆ alkoxyl). In certain embodiments, R⁶ is a 3-7 membered saturated or partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is a 3-7 membered saturated heterocyclyl containing 1 or 2 heteroatoms independently selected from oxygen and nitrogen, wherein the heterocyclyl is substituted with 0 or 1 occurrences of R¹². In certain embodiments, R⁶ is a 3-7 membered saturated heterocyclyl containing 1 heteroatom independently selected from oxygen and nitrogen, wherein the heterocyclyl is substituted with 0 or 1 occurrences of R¹². In certain embodiments, R⁶ is a 3-7 membered saturated heterocyclyl containing 2 heteroatoms independently selected from oxygen and nitrogen, wherein the heterocyclyl is substituted with 0 or 1 occurrences of R¹². In certain embodiments, R⁶ is a 3-7 membered partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is a 3-7 membered partially unsaturated heterocyclyl containing 1 heteroatom independently selected from oxygen and nitrogen, wherein the heterocyclyl is substituted with 0 or 1 occurrences of R¹². In certain embodiments, R⁶ is a 3-7 membered partially unsaturated heterocyclyl containing 2 heteroatoms independently selected from oxygen and nitrogen, wherein the heterocyclyl is substituted with 0 or 1 occurrences of R¹². In certain embodiments, R⁶ is a 4 membered saturated or partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is a 5 membered saturated or partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is a 6 membered saturated or partially unsaturated heterocyclyl containing 1 or 2 heteroatoms independently selected from oxygen and nitrogen, wherein the heterocyclyl is substituted with 0 or 1 occurrences of R¹². In certain embodiments, R⁶ is —C(CH₃)₂OCH₃. In certain embodiments, R⁶ is —CH(CH₃)OCH₃. In certain embodiments, R⁶ is —CH₂OH. In certain embodiments, R⁶ is

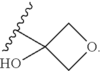 [SMILES: *C1(O)COC1]

In certain embodiments, R⁶ is

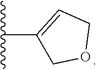 [SMILES: *C1=CCOC1]

In certain embodiments, R⁶ is

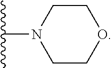 [SMILES: *N1CCOCC1]

In certain embodiments, R⁶ is

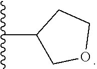 [SMILES: *C1CCOC1]

In certain embodiments, R⁶ is

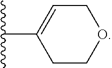 [SMILES: *C1=CCOCC1]

In certain embodiments, R⁶ is

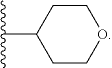 [SMILES: *C1CCOCC1]

In certain embodiments, R⁶ is

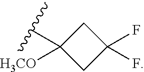 [SMILES: *C1(OC)CC(F)(F)C1]

In certain embodiments, R⁶ is

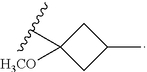 [SMILES: *C1(OC)CC(C)C1]

In certain embodiments, R⁶ is

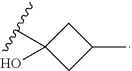 [SMILES: *C1(O)CC(C)C1]

In certain embodiments, R⁶ is

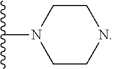

In certain embodiments, R⁶ is

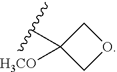 [SMILES: *C1(OC)COC1]

In certain embodiments, R⁶ is

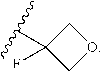 [SMILES: *C1(F)COC1]

In certain embodiments, R⁶ is selected from the groups depicted in the compounds in Table 1 below.

As defined generally above, R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 4 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 5 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is 6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₁ alkyl. In certain embodiments, R⁸ is C₂ alkyl. In certain embodiments, R⁸ is C₃ alkyl. In certain embodiments, R⁸ is C₄ alkyl. In certain embodiments, R⁸ is C₅ alkyl. In certain embodiments, R⁸ is C₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₁ alkyl. In certain embodiments, R⁹ is C₂ alkyl. In certain embodiments, R⁹ is C₃ alkyl. In certain embodiments, R⁹ is C₄ alkyl. In certain embodiments, R⁹ is C₅ alkyl. In certain embodiments, R⁹ is C₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or (C₀₋₅ alkylene)-C₃₋₇ cycloalkyl. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is (C₀₋₅ alkylene)-C₃₋₇ cycloalkyl. In certain embodiments, R¹⁰ is —C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ represents independently for each occurrence halo. In certain embodiments, R¹¹ represents independently for each occurrence fluoro. In certain embodiments, R¹¹ represents independently for each occurrence hydroxyl. In certain embodiments, R¹¹ represents independently for each occurrence C₁₋₆ alkyl. In certain embodiments, R¹¹ represents independently for each occurrence C₁ alkyl. In certain embodiments, R¹¹ represents independently for each occurrence C₂ alkyl. In certain embodiments, R¹¹ represents independently for each occurrence C₃ alkyl. In certain embodiments, R¹¹ represents independently for each occurrence C₄ alkyl. In certain embodiments, R¹¹ represents independently for each occurrence C₅alkyl. In certain embodiments, R¹¹ represents independently for each occurrence C₆ alkyl. In certain embodiments, R¹¹ represents independently for each occurrence C₁₋₆ haloalkyl. In certain embodiments, R¹¹ represents independently for each occurrence C₁ haloalkyl. In certain embodiments, R¹¹ represents independently for each occurrence C₂ haloalkyl. In certain embodiments, R¹¹ represents independently for each occurrence C₃ haloalkyl. In certain embodiments, R¹¹ represents independently for each occurrence C₄ haloalkyl. In certain embodiments, R¹¹ represents independently for each occurrence C₅ haloalkyl. In certain embodiments, R¹¹ represents independently for each occurrence C₆ haloalkyl. In certain embodiments, R¹¹ represents independently for each occurrence C₁₋₆ alkoxyl. In certain embodiments, R¹¹ represents independently for each occurrence C₁ alkoxyl. In certain embodiments, R¹¹ represents independently for each occurrence C₂ alkoxyl. In certain embodiments, R¹¹ represents independently for each occurrence C₃ alkoxyl. In certain embodiments, R¹¹ represents independently for each occurrence C₄ alkoxyl. In certain embodiments, R¹¹ represents independently for each occurrence C₅ alkoxyl. In certain embodiments, R¹¹ represents independently for each occurrence C₆ alkoxyl. In certain embodiments, R¹¹ represents independently for each occurrence C₃₋₇ cycloalkyl. In certain embodiments, R¹¹ is selected from the groups depicted in the compounds in Table 1 below.

As defined generally above, R¹² represents independently for each occurrence C₁₋₆ alkyl, C₁₋₆ alkoxyl, halo, or hydroxyl. In certain embodiments, R¹² represents independently for each occurrence C₁₋₆ alkyl. In certain embodiments, R¹² represents independently for each occurrence C₁ alkyl. In certain embodiments, R¹² represents independently for each occurrence C₂ alkyl. In certain embodiments, R¹² represents independently for each occurrence C₃ alkyl. In certain embodiments, R¹² represents independently for each occurrence C₄ alkyl. In certain embodiments, R¹² represents independently for each occurrence C₅alkyl. In certain embodiments, R¹² represents independently for each occurrence C₆ alkyl. In certain embodiments, R¹² represents independently for each occurrence C₁₋₆ alkoxyl. In certain embodiments, R¹² represents independently for each occurrence C₁ alkoxyl. In certain embodiments, R¹² represents independently for each occurrence C₂ alkoxyl. In certain embodiments, R¹² represents independently for each occurrence C₃ alkoxyl. In certain embodiments, R¹² represents independently for each occurrence C₄ alkoxyl. In certain embodiments, R¹² represents independently for each occurrence C₅ alkoxyl. In certain embodiments, R¹² represents independently for each occurrence C₆ alkoxyl. In certain embodiments, R¹² represents independently for each occurrence halo. In certain embodiments, R¹² represents independently for each occurrence fluoro. In certain embodiments, R¹² represents independently for each occurrence hydroxyl. In certain embodiments, R¹² is selected from the groups depicted in the compounds in Table 1 below.

As defined generally above, n, m, x, and y are independently 0, 1, or 2. In certain embodiments, n is 0. In certain embodiments, n is 1. In certain embodiments, n is 2. In certain embodiments, m is 0. In certain embodiments, m is 1. In certain embodiments, m is 2. In certain embodiments, x is 0. In certain embodiments, x is 1. In certain embodiments, x is 2. In certain embodiments, y is 0. In certain embodiments, y is 1. In certain embodiments, y is 2.

As defined generally above, q is 0, 1, 2, or 3. In certain embodiments q is 0. In certain embodiments q is 1. In certain embodiments q is 2. In certain embodiments q is 3.

The description above describes multiple embodiments relating to compounds of Formula I-2. The patent application specifically contemplates all combinations of the embodiments.

Another aspect of the invention provides a compound represented by Formula I-2

### Part B:

Another aspect of the invention provides a compound represented by Formula I-3:

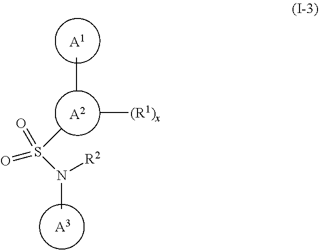

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteratoms independently selected from oxygen, nitrogen, and sulfur, wherein the heteroaryl and heterocyclyl are substituted with n occurrences of R⁶;
A² is pyrazolylene or 1,2,3-triazolylene;
A³ is

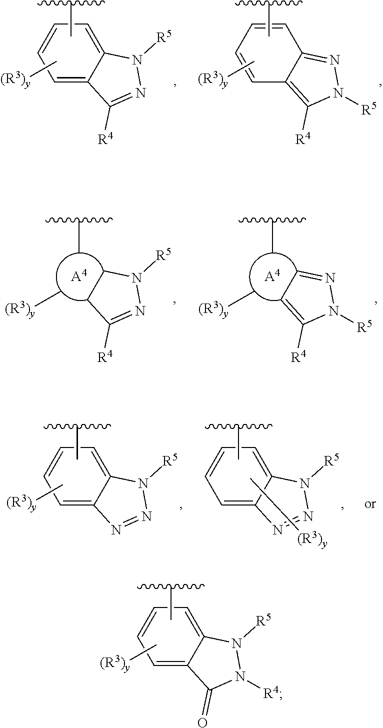

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R′)S(O₂)R¹⁰;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

The definitions of variables in Formula I-3 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).′

In certain embodiments, the compound is a compound of Formula I-3.

As defined generally above, A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶. In certain embodiments, A¹ is a 5-6 membered heteroaryl containing 1, 2, or 3 heteratoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl are substituted with n occurrences of R⁶. In certain embodiments, A¹ is a 6-membered heteroaryl containing 1, 2, or 3 heteratoms independently selected from oxygen, nitrogen, and sulfur, wherein the heteroaryl is substituted with n occurrences of R⁶. In certain embodiments, A¹ is pyridinyl, pyrimidinyl, pyridazinyl, or pyrazinyl, each of which is substituted with n occurrences of R⁶. In certain embodiments, A¹ is pyridinyl substituted with n occurrences of R⁶. In certain embodiments, A¹ is

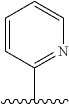 [SMILES: *c1ccccn1]

substituted with n occurrences of R⁶. In certain embodiments, A¹ is

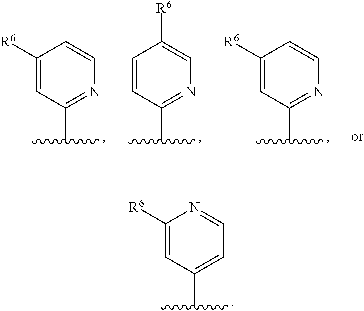

In certain embodiments, A¹ is

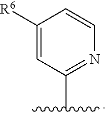

In certain embodiments, A¹ is

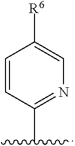

In certain embodiments, A is

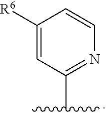

In certain embodiments, A¹ is

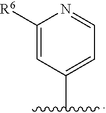

In certain embodiments, A¹ is is

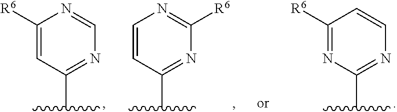

In certain embodiments, A¹ is

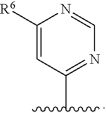

In certain embodiments, A¹ is

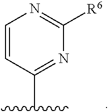

In certain embodiments, A¹ is

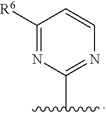

In certain embodiments, A¹ is a 5-membered heteroaryl containing 1, 2, or 3 heteratoms independently selected from oxygen, nitrogen, and sulfur, wherein the heteroaryl is substituted with n occurrences of R⁶. In certain embodiments, A¹ is a 9-10 membered bicyclic heteroaryl containing 1, 2, or 3 heteroatoms independently selected from oxygen, nitrogen, and sulfur. In certain embodiments, A¹ is a 9-10 membered bicyclic heteroaryl containing 1 heteroatom selected from oxygen, nitrogen, and sulfur. In certain embodiments, A¹ is a 9-10 membered bicyclic heteroaryl containing 2 heteroatoms independently selected from oxygen, nitrogen, and sulfur. In certain embodiments, A¹ is a 9-10 membered bicyclic heteroaryl containing 3 heteroatoms independently selected from oxygen, nitrogen, and sulfur. In certain embodiments, A¹ is a 3-10 membered saturated heterocyclyl containing 1, 2, or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom independently selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶. In certain embodiments, A¹ is selected from the groups depicted in the compounds in Table 1 below.

As defined generally above, A² is pyrazolylene or 1,2,3-triazolylene. In certain embodiments, A² is pyrazolylene. In certain embodiments, A² is 1,2,3-triazolylene.

In certain embodiments, A² is

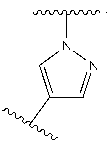 [SMILES: *c1cnn(*)c1]

In certain embodiments, A² is

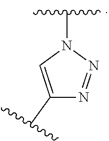 [SMILES: *c1cn(*)nn1]

In certain embodiments, A² is selected from the groups depicted in the compounds in Table 1 below. As defined generally above, A³ is

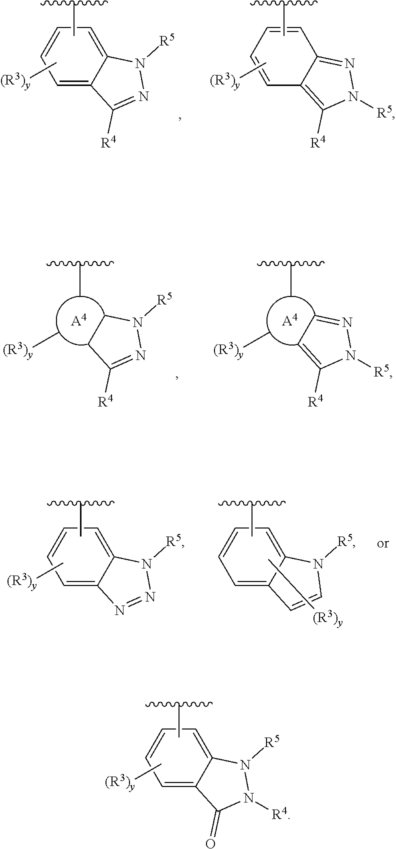

In certain embodiments, A³ is

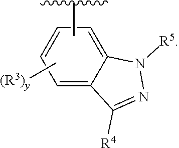

In certain embodiments, A³ is

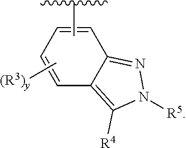

In certain embodiments, A³ is

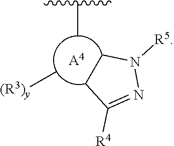

In certain embodiments, A³ is

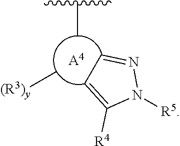

In certain embodiments, A³ is

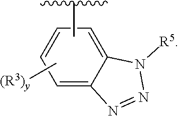

In certain embodiments, A³ is

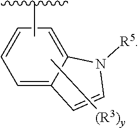

In certain embodiments, A³ is

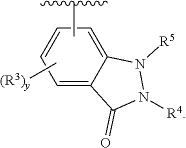

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R⁵ is C₁. 4 alkyl. In certain embodiments, R⁵ is methyl. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl In certain embodiments, R³ is C₁₋₆ haloalkyl. In certain embodiments, R³ is C₁₋₆ deuteroalkyl. In certain embodiments, R³ is C₁₋₆ deuteroalkoxyl. In certain embodiments, R³ is C₃₋₇ halocycloalkyl. In certain embodiments, R³ is C₃₋₇ hydroxycycloalkyl. In certain embodiments, R³ is —O—C₃₋₇ cycloalkyl. In certain embodiments, R³ is —(C₀₋₄ alkylene)-CN. In certain embodiments, R³ is cyano. In certain embodiments, R³ is C₂₋₄ alkynyl. In certain embodiments, R³ is —N(R⁸)(R⁹). In certain embodiments, R³ is CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, R³ is a 3-7 membered saturated heterocyclyl containing 1 or 2 heteroatoms independently selected from oxygen and nitrogen, wherein the heterocyclyl is substituted with 0 or 1 halo. In certain embodiments, R³ is a 3-7 membered saturated heterocyclyl containing 1 heteroatom selected from oxygen and nitrogen, wherein the heterocyclyl is substituted with 0 or 1 halo. In certain embodiments, R³ is a 3-7 membered saturated heterocyclyl containing 2 heteroatoms independently selected from oxygen and nitrogen, wherein the heterocyclyl is substituted with 0 or 1 halo. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is F. In certain embodiments, R⁴ is Cl. In certain embodiments, R⁴ is Br. In certain embodiments, R⁴ is I. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R⁸)S(O₂)R¹⁰. In certain embodiments, R⁶ is C₁₋₆ haloalkyl. In certain embodiments, R⁶ is —CF₃. In certain embodiments, R⁶ is C₁₋₆ alkyl. In certain embodiments, R⁶ is methyl. In certain embodiments, R⁶ is C₃₋₇ cycloalkyl. In certain embodiments, R⁶ is cyclopropyl. In certain embodiments, R⁶ is halo. In certain embodiments, R⁶ is hydroxyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —N(R⁸)(R⁹). In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is selected from the groups depicted in the compounds in Table 1 below.

As defined generally above, R⁷ is —OH, —O—(C₁₋₆ alkyl), or —O—C₃₋₇ cycloalkyl. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or (C₀₋₅ alkylene)-C₃₋₇ cycloalkyl. In certain embodiments, R¹⁰ is C₁₋₆ alkyl. In certain embodiments, R¹⁰ is (C₀₋₅ alkylene)-C₃₋₇ cycloalkyl. In certain embodiments, R¹⁰ is selected from the groups depicted in the compounds in Table 1 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 1 below.

As defined generally above, n, m, and x are independently 0, 1, or 2. In certain embodiments, n and x are independently 0, 1, or 2. In certain embodiments, x is 0. In certain embodiments, n is 1. In certain embodiments, n is 2. In certain embodiments, n is 0. In certain embodiments, x is 1. In certain embodiments, x is 2. In certain embodiments, m is 0. In certain embodiments, m is 1. In certain embodiments, m is 2. In certain embodiments, n is selected from the corresponding value in the groups depicted in the compounds in Table 1 below. In certain embodiments, m is selected from the corresponding value in the groups depicted in the compounds in Table 1 below. In certain embodiments, x is selected from the corresponding value in the groups depicted in the compounds in Table 1 below.

The description above describes multiple embodiments relating to compounds of Formula I-3. The patent application specifically contemplates all combinations of the embodiments.

In certain embodiments, said compound is defined by the Formula I-4:

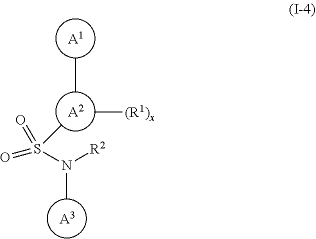

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is pyrazolylene or 1,2,3-triazolylene;
A³ is

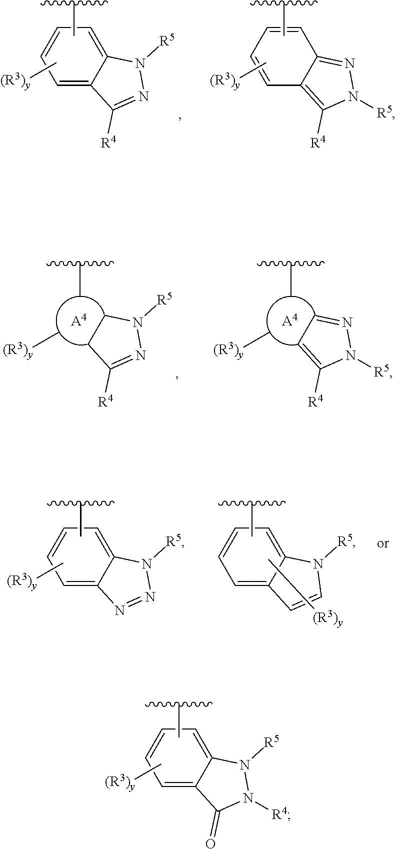

A⁴ is a 6-membered aromatic ring containing 1 nitrogen atom;
R¹ represents independently for each occurrence halo, C₁₋₄ alkyl, C₁₋₄ halolkyl, or cyano; R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R′)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R′)S(O₂)R¹⁰;
R⁷ is —OH, —O—(C₁₋₆ alkyl), or —O—C₃₋₇ cycloalkyl;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
y is 0, 1, or 2; and
n and x are independently 0, 1, or 2

The description above describes multiple embodiments relating to compounds of Formula I-4. The patent application specifically contemplates all combinations of the embodiments.

In certain embodiments, the compound of Formula I-4 is further defined by Formula Ia-4:

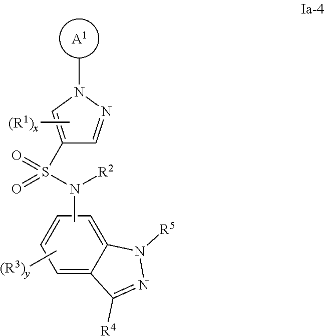

or a pharmaceutically acceptable salt thereof. In certain embodiments, the definition of variables R¹, R², R³, R⁴, R⁵, A¹, x, and y is one of the embodiments described above in connection with Formula I-4.

The description above describes multiple embodiments relating to compounds of Formula Ia-4. The patent application specifically contemplates all combinations of the embodiments.

In certain embodiments, the compound of Formula I-4 is further defined by Formula Ib-4 or Ic-4:

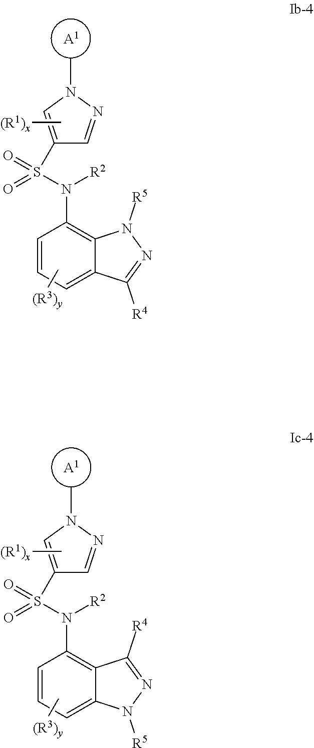

or a pharmaceutically acceptable salt thereof. In certain embodiments, the definition of variables R¹, R², R³, R⁴, R⁵, A¹, x, and y is one of the embodiments described above in connection with Formula I-4. In certain embodiments, the compound of Formula I-4 is further defined by Formula Ib-4 or a pharmaceutically acceptable salt thereof. In certain embodiments, the compound of Formula I-4 is further defined by Formula Ic-4 or a pharmaceutically acceptable salt thereof.

The description above describes multiple embodiments relating to compounds of Formulae Ia-4 and Ib-4. The patent application specifically contemplates all combinations of the embodiments.

In certain embodiments, the compound of Formula I-4 is further defined by Formula Id-4 or Ie-4:

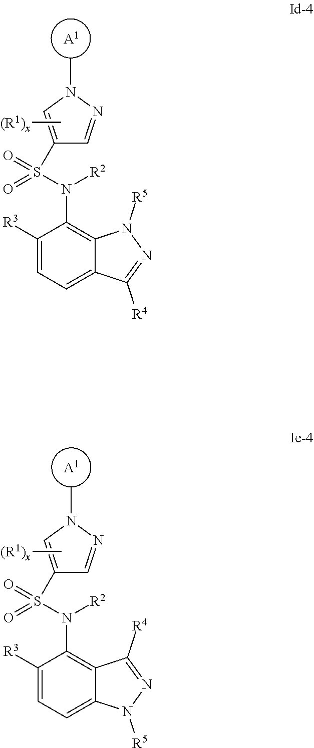

or a pharmaceutically acceptable salt thereof. In certain embodiments, the definition of variables R¹, R², R³, R⁴, R⁵, A¹, and x is one of the embodiments described above in connection with Formula I-4. In certain embodiments, the compound of Formula I-4 is further defined by Formula Id-4 or a pharmaceutically acceptable salt thereof. In certain embodiments, the compound of Formula I-4 is further defined by Formula Ie-4 or a pharmaceutically acceptable salt thereof.

The description above describes multiple embodiments relating to compounds of Formulae Id-4 and Ie-4. The patent application specifically contemplates all combinations of the embodiments.

In certain embodiments, the compound of Formula I-4 is further defined by Formula If-4:

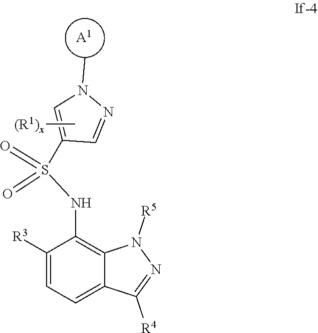

or a pharmaceutically acceptable salt thereof. In certain embodiments, the definition of variables R¹, R³, R⁴, R⁵, A¹, and x is one of the embodiments described above in connection with Formula I-4.

The description above describes multiple embodiments relating to compounds of Formula If-4. The patent application specifically contemplates all combinations of the embodiments.

In certain embodiments, the compound of Formula I-4 is further defined by Formula Ig-4 or Ih-4:

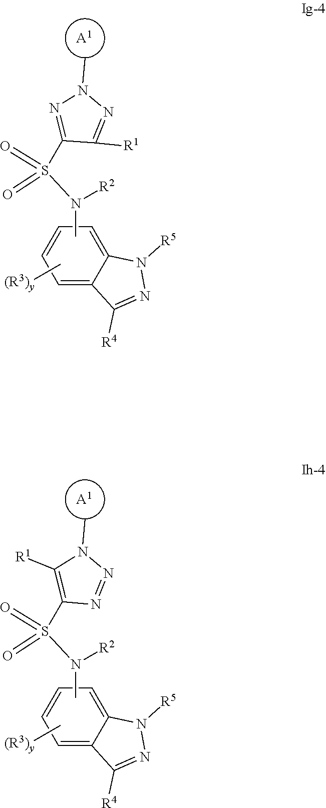

or a pharmaceutically acceptable salt thereof. In certain embodiments, the definition of variables R¹, R², R³, R⁴, R⁵, A¹, and y is one of the embodiments described above in connection with Formula I-4. In certain embodiments, the compound of Formula I-4 is further defined by Formula Ig-4 or a pharmaceutically acceptable salt thereof. In certain embodiments, the compound of Formula I-4 is further defined by Formula Ih-4 or a pharmaceutically acceptable salt thereof.

The description above describes multiple embodiments relating to compounds of Formulae Ig-4 and Ih-4. The patent application specifically contemplates all combinations of the embodiments.

In certain embodiments, the compound of Formula I-4 is further defined by Formula Ii-4, Ij-4, Ik-4, or Il-4:

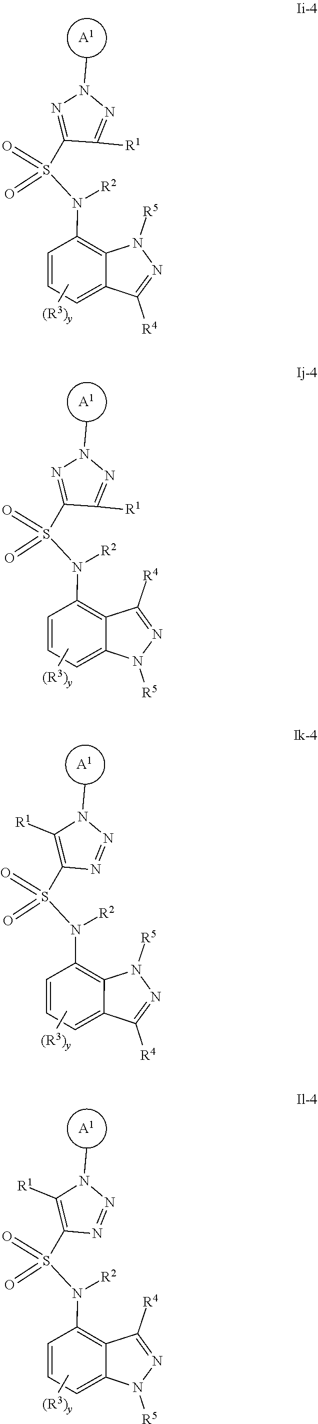

or a pharmaceutically acceptable salt thereof. In certain embodiments, the definition of variables R¹, R², R³, R⁴, R⁵, A¹, and y is one of the embodiments described above in connection with Formula I-4. In certain embodiments, the compound of Formula I-4 is further defined by Formula Ii-4 or a pharmaceutically acceptable salt thereof. In certain embodiments, the compound of Formula I-4 is further defined by Formula Ij-4 or a pharmaceutically acceptable salt thereof. In certain embodiments, the compound of Formula I-4 is further defined by Formula Ik-4 or a pharmaceutically acceptable salt thereof. In certain embodiments, the compound of Formula I-4 is further defined by Formula Il-4 or a pharmaceutically acceptable salt thereof.

The description above describes multiple embodiments relating to compounds of Formulae Ii-4, Ij-4, Ik-4, and Il-4. The patent application specifically contemplates all combinations of the embodiments.

In certain embodiments, the compound of Formula I-4 is further defined by Formula Im-4, In-4, Io-4, or Ip-4:

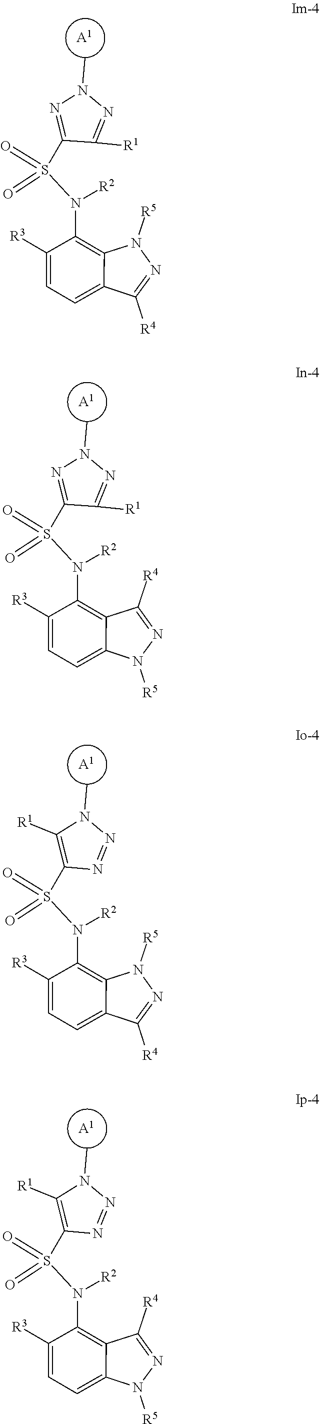

or a pharmaceutically acceptable salt thereof. In certain embodiments, the definition of variables R¹, R³, R⁴, R⁵, and A¹ is one of the embodiments described above in connection with Formula I-1. In certain embodiments, the compound of Formula I-4 is further defined by Formula Im-4 or a pharmaceutically acceptable salt thereof. In certain embodiments, the compound of Formula I-4 is further defined by Formula In-4 or a pharmaceutically acceptable salt thereof. In certain embodiments, the compound of Formula I-4 is further defined by Formula Io-4 or a pharmaceutically acceptable salt thereof. In certain embodiments, the compound of Formula I-4 is further defined by Formula Ip-4 or a pharmaceutically acceptable salt thereof.

The description above describes multiple embodiments relating to compounds of Formulae Im-4, In-4, Io-4, and Ip-4. The patent application specifically contemplates all combinations of the embodiments.

### Part C:

Another aspect of the invention provides a compound in Table 1 below, or a pharmaceutically acceptable salt thereof. In certain embodiments, the compound is a compound in Table 1 below. In certain embodiments, the compound is any one of compound I-1 to I-90 in Table 1 below, or a pharmaceutically acceptable salt thereof. In certain embodiments, the compound is any one of compound I-1 to I-90 in Table 1 below. In certain embodiments, the compound is any one of compound I-91 to to I-485 in Table 1 below, or a pharmaceutically acceptable salt thereof. In certain embodiments, the compound is any one of compound I-91 to to I-485 in Table 1 below.

TABLE 1

| Compound No. | Stucture |
|---|---|
| I-1 | 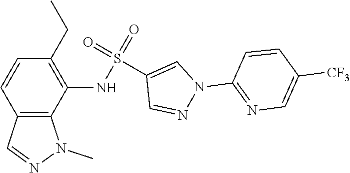 [SMILES: CCc1ccc2cnn(C)c2c1NS(=O)(=O)c1cnn(-c2ccc(C(F)(F)F)cn2)c1] |
| I-2 | 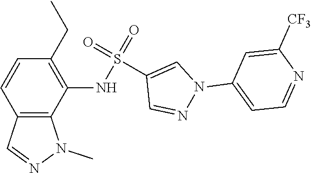 [SMILES: CCc1ccc2cnn(C)c2c1NS(=O)(=O)c1cnn(-c2ccnc(C(F)(F)F)c2)c1] |
| I-3 | 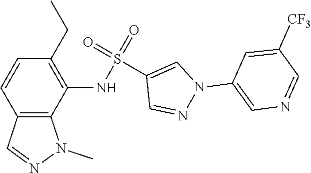 [SMILES: CCc1ccc2cnn(C)c2c1NS(=O)(=O)c1cnn(-c2cncc(C(F)(F)F)c2)c1] |
| I-4 | 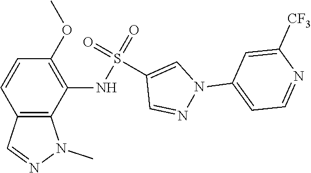 [SMILES: COc1ccc2cnn(C)c2c1NS(=O)(=O)c1cnn(-c2ccnc(C(F)(F)F)c2)c1] |
| I-5 | 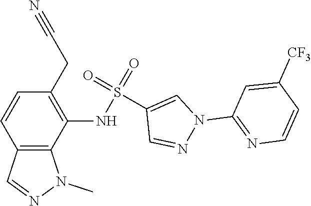 [SMILES: Cn1ncc2ccc(CC#N)c(NS(=O)(=O)c3cnn(-c4cc(C(F)(F)F)ccn4)c3)c21] |
| I-6 | 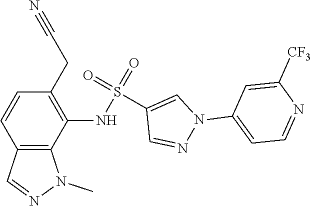 [SMILES: Cn1ncc2ccc(CC#N)c(NS(=O)(=O)c3cnn(-c4ccnc(C(F)(F)F)c4)c3)c21] |
| I-7 | 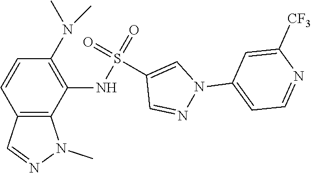 [SMILES: CN(C)c1ccc2cnn(C)c2c1NS(=O)(=O)c1cnn(-c2ccnc(C(F)(F)F)c2)c1] |
| I-8 | 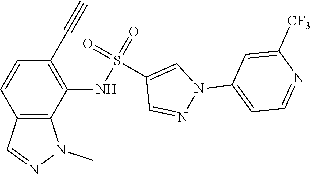 [SMILES: C#Cc1ccc2cnn(C)c2c1NS(=O)(=O)c1cnn(-c2ccnc(C(F)(F)F)c2)c1] |
| I-9 | 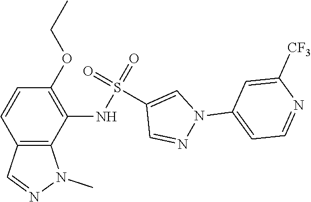 [SMILES: CCOc1ccc2cnn(C)c2c1NS(=O)(=O)c1cnn(-c2ccnc(C(F)(F)F)c2)c1] |
| I-10 | 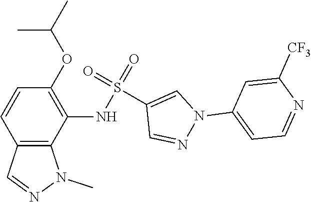 [SMILES: CC(C)Oc1ccc2cnn(C)c2c1NS(=O)(=O)c1cnn(-c2ccnc(C(F)(F)F)c2)c1] |
| I-11 | 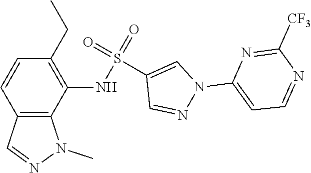 [SMILES: CCc1ccc2cnn(C)c2c1NS(=O)(=O)c1cnn(-c2ccnc(C(F)(F)F)n2)c1] |
| I-12 | 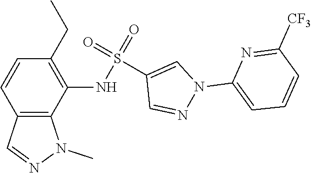 [SMILES: CCc1ccc2cnn(C)c2c1NS(=O)(=O)c1cnn(-c2cccc(C(F)(F)F)n2)c1] |
| I-13 | 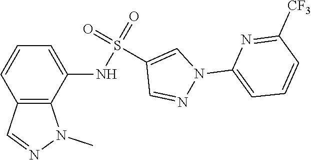 [SMILES: Cn1ncc2cccc(NS(=O)(=O)c3cnn(-c4cccc(C(F)(F)F)n4)c3)c21] |
| I-14 | 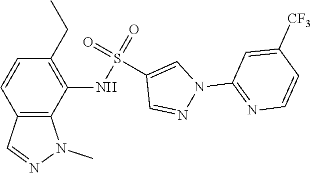 [SMILES: CCc1ccc2cnn(C)c2c1NS(=O)(=O)c1cnn(-c2cc(C(F)(F)F)ccn2)c1] |
| I-15 | 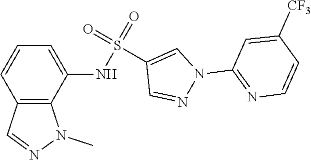 [SMILES: Cn1ncc2cccc(NS(=O)(=O)c3cnn(-c4cc(C(F)(F)F)ccn4)c3)c21] |
| I-16 | 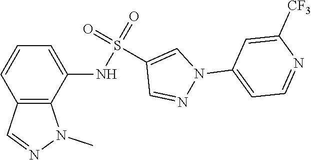 [SMILES: Cn1ncc2cccc(NS(=O)(=O)c3cnn(-c4ccnc(C(F)(F)F)c4)c3)c21] |
| I-17 | 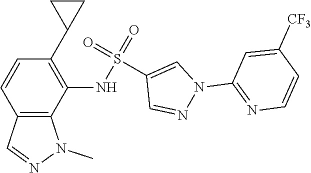 [SMILES: Cn1ncc2ccc(C3CC3)c(NS(=O)(=O)c3cnn(-c4cc(C(F)(F)F)ccn4)c3)c21] |
| I-18 | 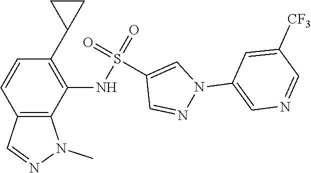 [SMILES: Cn1ncc2ccc(C3CC3)c(NS(=O)(=O)c3cnn(-c4cncc(C(F)(F)F)c4)c3)c21] |
| I-19 | 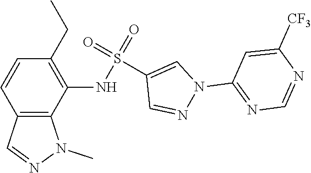 [SMILES: CCc1ccc2cnn(C)c2c1NS(=O)(=O)c1cnn(-c2cc(C(F)(F)F)ncn2)c1] |
| I-20 | 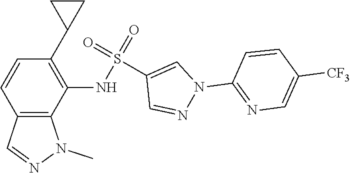 [SMILES: Cn1ncc2ccc(C3CC3)c(NS(=O)(=O)c3cnn(-c4ccc(C(F)(F)F)cn4)c3)c21] |
| I-21 | 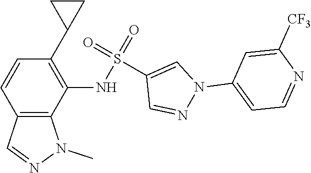 [SMILES: Cn1ncc2ccc(C3CC3)c(NS(=O)(=O)c3cnn(-c4ccnc(C(F)(F)F)c4)c3)c21] |
| I-22 | 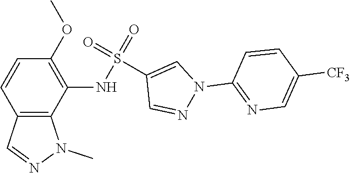 [SMILES: COc1ccc2cnn(C)c2c1NS(=O)(=O)c1cnn(-c2ccc(C(F)(F)F)cn2)c1] |
| I-23 | 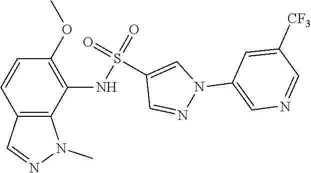 [SMILES: COc1ccc2cnn(C)c2c1NS(=O)(=O)c1cnn(-c2cncc(C(F)(F)F)c2)c1] |
| I-24 | 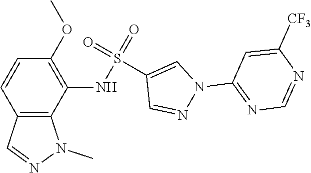 [SMILES: COc1ccc2cnn(C)c2c1NS(=O)(=O)c1cnn(-c2cc(C(F)(F)F)ncn2)c1] |
| I-25 | 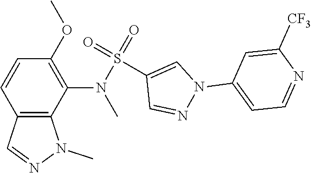 [SMILES: COc1ccc2cnn(C)c2c1N(C)S(=O)(=O)c1cnn(-c2ccnc(C(F)(F)F)c2)c1] |
| I-26 | 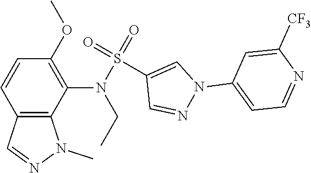 [SMILES: CCN(c1c(OC)ccc2cnn(C)c12)S(=O)(=O)c1cnn(-c2ccnc(C(F)(F)F)c2)c1] |
| I-27 | 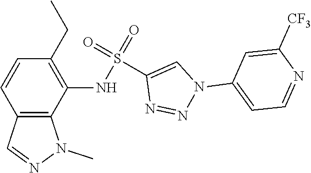 [SMILES: CCc1ccc2cnn(C)c2c1NS(=O)(=O)c1cn(-c2ccnc(C(F)(F)F)c2)nn1] |
| I-28 | 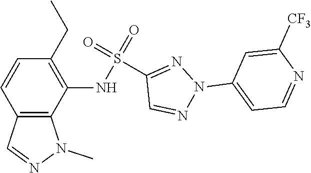 [SMILES: CCc1ccc2cnn(C)c2c1NS(=O)(=O)c1cnn(-c2ccnc(C(F)(F)F)c2)n1] |
| I-29 | 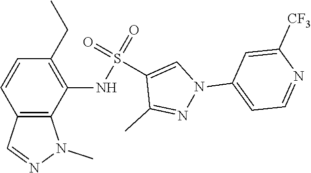 [SMILES: CCc1ccc2cnn(C)c2c1NS(=O)(=O)c1cn(-c2ccnc(C(F)(F)F)c2)nc1C] |
| I-30 | 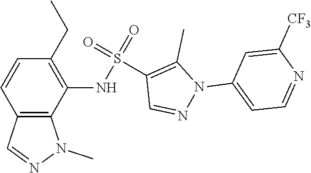 [SMILES: CCc1ccc2cnn(C)c2c1NS(=O)(=O)c1cnn(-c2ccnc(C(F)(F)F)c2)c1C] |
| I-31 | 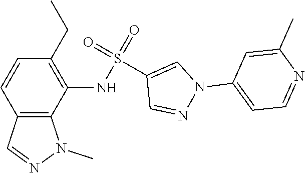 [SMILES: CCc1ccc2cnn(C)c2c1NS(=O)(=O)c1cnn(-c2ccnc(C)c2)c1] |
| I-32 | 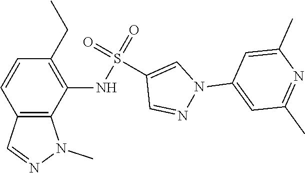 [SMILES: CCc1ccc2cnn(C)c2c1NS(=O)(=O)c1cnn(-c2cc(C)nc(C)c2)c1] |
| I-33 | 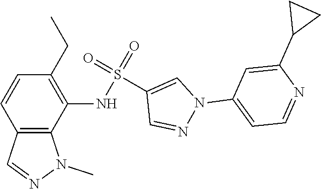 [SMILES: CCc1ccc2cnn(C)c2c1NS(=O)(=O)c1cnn(-c2ccnc(C3CC3)c2)c1] |
| I-34 | 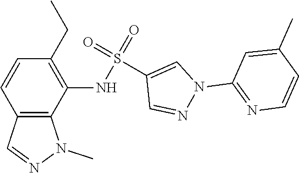 [SMILES: CCc1ccc2cnn(C)c2c1NS(=O)(=O)c1cnn(-c2cc(C)ccn2)c1] |
| I-35 | 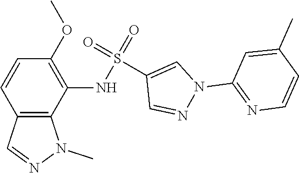 [SMILES: COc1ccc2cnn(C)c2c1NS(=O)(=O)c1cnn(-c2cc(C)ccn2)c1] |
| I-36 | 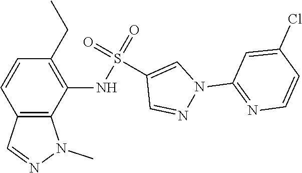 [SMILES: CCc1ccc2cnn(C)c2c1NS(=O)(=O)c1cnn(-c2cc(Cl)ccn2)c1] |
| I-37 | 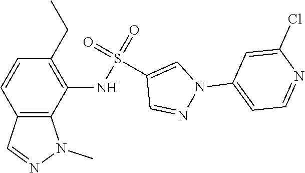 [SMILES: CCc1ccc2cnn(C)c2c1NS(=O)(=O)c1cnn(-c2ccnc(Cl)c2)c1] |
| I-38 | 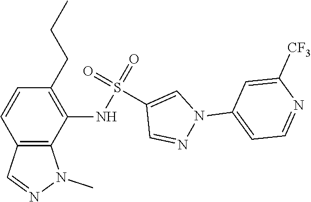 [SMILES: CCCc1ccc2cnn(C)c2c1NS(=O)(=O)c1cnn(-c2ccnc(C(F)(F)F)c2)c1] |
| I-39 | 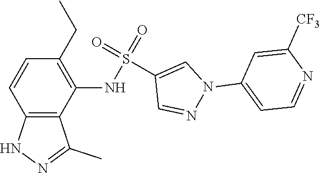 [SMILES: CCc1ccc2[nH]nc(C)c2c1NS(=O)(=O)c1cnn(-c2ccnc(C(F)(F)F)c2)c1] |
| I-40 | 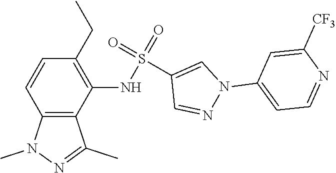 [SMILES: CCc1ccc2c(c(C)nn2C)c1NS(=O)(=O)c1cnn(-c2ccnc(C(F)(F)F)c2)c1] |
| I-41 | 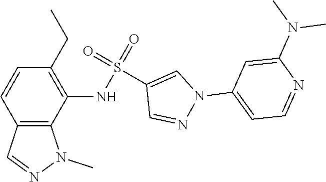 [SMILES: CCc1ccc2cnn(C)c2c1NS(=O)(=O)c1cnn(-c2ccnc(N(C)C)c2)c1] |
| I-42 | 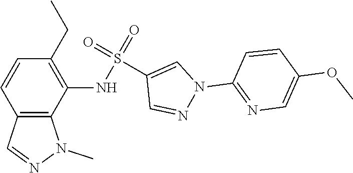 [SMILES: CCc1ccc2cnn(C)c2c1NS(=O)(=O)c1cnn(-c2ccc(OC)cn2)c1] |
| I-43 | 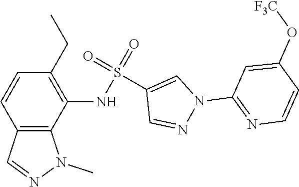 [SMILES: CCc1ccc2cnn(C)c2c1NS(=O)(=O)c1cnn(-c2cc(OC(F)(F)F)ccn2)c1] |
| I-44 | 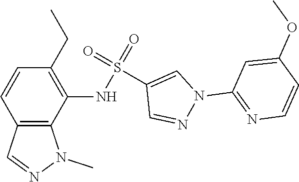 [SMILES: CCc1ccc2cnn(C)c2c1NS(=O)(=O)c1cnn(-c2cc(OC)ccn2)c1] |
| I-45 | 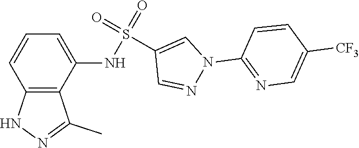 [SMILES: Cc1n[nH]c2cccc(NS(=O)(=O)c3cnn(-c4ccc(C(F)(F)F)cn4)c3)c12] |
| I-46 | 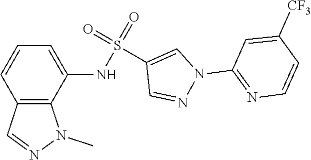 [SMILES: Cn1ncc2cccc(NS(=O)(=O)c3cnn(-c4cc(C(F)(F)F)ccn4)c3)c21] |
| I-47 | 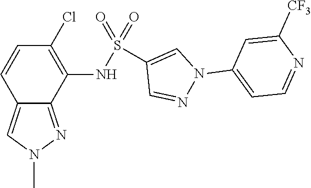 [SMILES: Cn1cc2ccc(Cl)c(NS(=O)(=O)c3cnn(-c4ccnc(C(F)(F)F)c4)c3)c2n1] |
| I-48 | 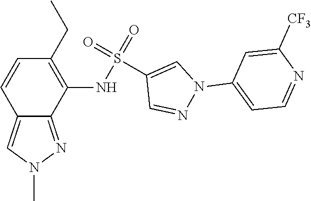 [SMILES: CCc1ccc2cn(C)nc2c1NS(=O)(=O)c1cnn(-c2ccnc(C(F)(F)F)c2)c1] |
| I-49 | 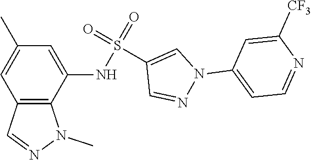 [SMILES: Cc1cc(NS(=O)(=O)c2cnn(-c3ccnc(C(F)(F)F)c3)c2)c2c(cnn2C)c1] |
| I-50 | 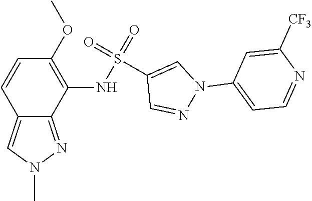 [SMILES: COc1ccc2cn(C)nc2c1NS(=O)(=O)c1cnn(-c2ccnc(C(F)(F)F)c2)c1] |
| I-51 | 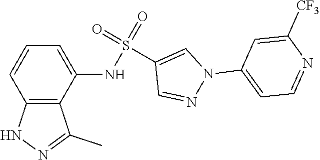 [SMILES: Cc1n[nH]c2cccc(NS(=O)(=O)c3cnn(-c4ccnc(C(F)(F)F)c4)c3)c12] |
| I-52 | 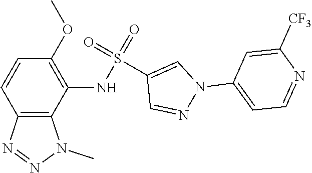 [SMILES: COc1ccc2nnn(C)c2c1NS(=O)(=O)c1cnn(-c2ccnc(C(F)(F)F)c2)c1] |
| I-53 | 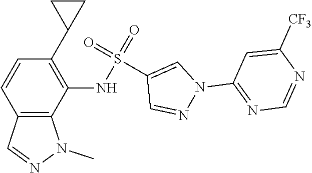 [SMILES: Cn1ncc2ccc(C3CC3)c(NS(=O)(=O)c3cnn(-c4cc(C(F)(F)F)ncn4)c3)c21] |
| I-54 | 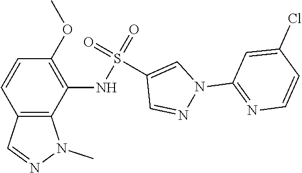 [SMILES: COc1ccc2cnn(C)c2c1NS(=O)(=O)c1cnn(-c2cc(Cl)ccn2)c1] |
| I-55 | 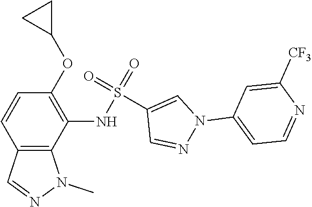 [SMILES: Cn1ncc2ccc(OC3CC3)c(NS(=O)(=O)c3cnn(-c4ccnc(C(F)(F)F)c4)c3)c21] |
| I-56 | 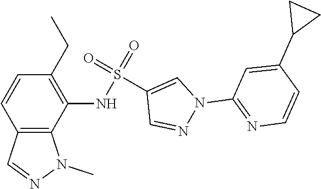 [SMILES: CCc1ccc2cnn(C)c2c1NS(=O)(=O)c1cnn(-c2cc(C3CC3)ccn2)c1] |
| I-57 | 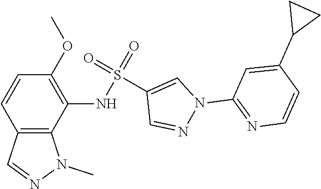 [SMILES: COc1ccc2cnn(C)c2c1NS(=O)(=O)c1cnn(-c2cc(C3CC3)ccn2)c1] |
| I-58 | 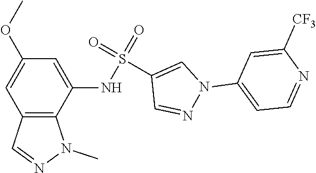 [SMILES: COc1cc(NS(=O)(=O)c2cnn(-c3ccnc(C(F)(F)F)c3)c2)c2c(cnn2C)c1] |
| I-59 | 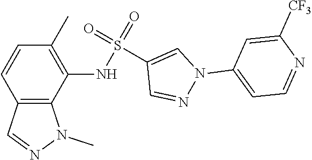 [SMILES: Cc1ccc2cnn(C)c2c1NS(=O)(=O)c1cnn(-c2ccnc(C(F)(F)F)c2)c1] |
| I-60 | 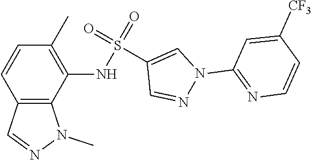 [SMILES: Cc1ccc2cnn(C)c2c1NS(=O)(=O)c1cnn(-c2cc(C(F)(F)F)ccn2)c1] |
| I-61 | 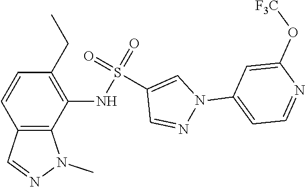 [SMILES: CCc1ccc2cnn(C)c2c1NS(=O)(=O)c1cnn(-c2ccnc(OC(F)(F)F)c2)c1] |
| I-62 | 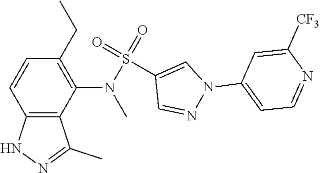 [SMILES: CCc1ccc2[nH]nc(C)c2c1N(C)S(=O)(=O)c1cnn(-c2ccnc(C(F)(F)F)c2)c1] |
| I-63 | 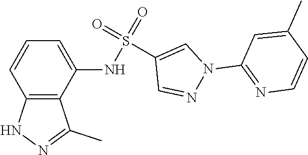 [SMILES: Cc1ccnc(-n2cc(S(=O)(=O)Nc3cccc4[nH]nc(C)c34)cn2)c1] |
| I-64 | 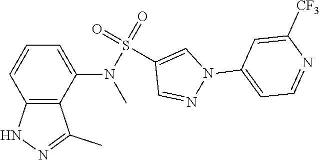 [SMILES: Cc1n[nH]c2cccc(N(C)S(=O)(=O)c3cnn(-c4ccnc(C(F)(F)F)c4)c3)c12] |
| I-65 | 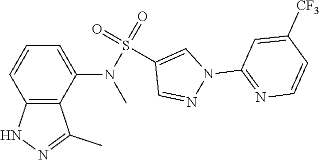 [SMILES: Cc1n[nH]c2cccc(N(C)S(=O)(=O)c3cnn(-c4cc(C(F)(F)F)ccn4)c3)c12] |
| I-66 | 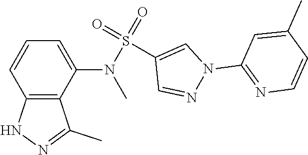 [SMILES: Cc1ccnc(-n2cc(S(=O)(=O)N(C)c3cccc4[nH]nc(C)c34)cn2)c1] |
| I-67 | 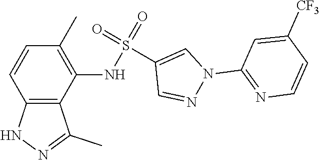 [SMILES: Cc1ccc2[nH]nc(C)c2c1NS(=O)(=O)c1cnn(-c2cc(C(F)(F)F)ccn2)c1] |
| I-68 | 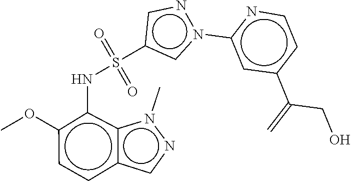 |
| I-69 | 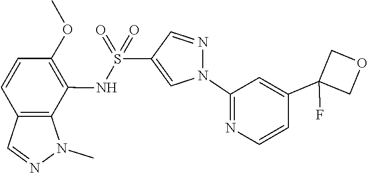 [SMILES: COc1ccc2cnn(C)c2c1NS(=O)(=O)c1cnn(-c2cc(C3(F)COC3)ccn2)c1] |
| I-70 | 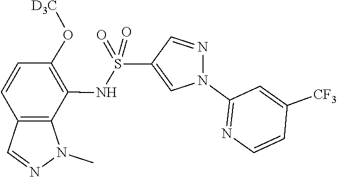 [SMILES: [2H]C([2H])([2H])Oc1ccc2cnn(C)c2c1NS(=O)(=O)c1cnn(-c2cc(C(F)(F)F)ccn2)c1] |
| I-71 | 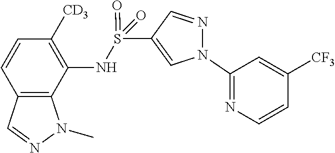 [SMILES: [2H]C([2H])([2H])c1ccc2cnn(C)c2c1NS(=O)(=O)c1cnn(-c2cc(C(F)(F)F)ccn2)c1] |
| I-72 | 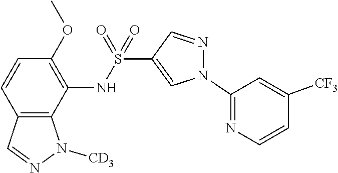 [SMILES: [2H]C([2H])([2H])n1ncc2ccc(OC)c(NS(=O)(=O)c3cnn(-c4cc(C(F)(F)F)ccn4)c3)c21] |
| I-73 | 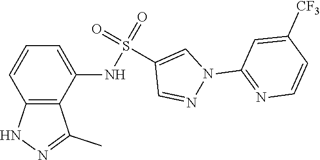 [SMILES: Cc1n[nH]c2cccc(NS(=O)(=O)c3cnn(-c4cc(C(F)(F)F)ccn4)c3)c12] |
| I-74 | 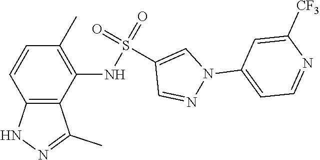 [SMILES: Cc1ccc2[nH]nc(C)c2c1NS(=O)(=O)c1cnn(-c2ccnc(C(F)(F)F)c2)c1] |
| I-75 | 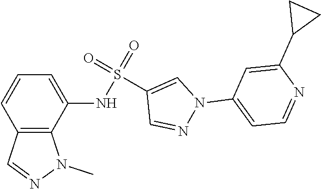 [SMILES: Cn1ncc2cccc(NS(=O)(=O)c3cnn(-c4ccnc(C5CC5)c4)c3)c21] |
| I-76 | 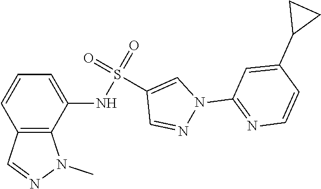 [SMILES: Cn1ncc2cccc(NS(=O)(=O)c3cnn(-c4cc(C5CC5)ccn4)c3)c21] |
| I-77 | 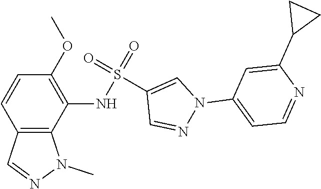 [SMILES: COc1ccc2cnn(C)c2c1NS(=O)(=O)c1cnn(-c2ccnc(C3CC3)c2)c1] |
| I-78 | 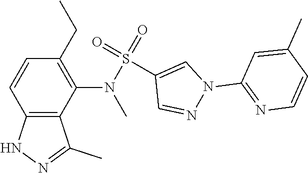 [SMILES: CCc1ccc2[nH]nc(C)c2c1N(C)S(=O)(=O)c1cnn(-c2cc(C)ccn2)c1] |
| I-79 | 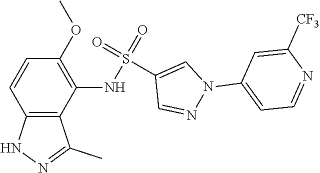 [SMILES: COc1ccc2[nH]nc(C)c2c1NS(=O)(=O)c1cnn(-c2ccnc(C(F)(F)F)c2)c1] |
| I-80 | 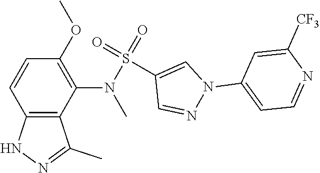 [SMILES: COc1ccc2[nH]nc(C)c2c1N(C)S(=O)(=O)c1cnn(-c2ccnc(C(F)(F)F)c2)c1] |
| I-81 | 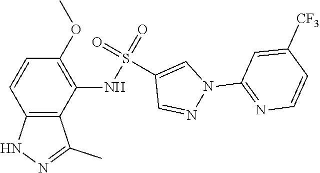 [SMILES: COc1ccc2[nH]nc(C)c2c1NS(=O)(=O)c1cnn(-c2cc(C(F)(F)F)ccn2)c1] |
| I-82 | 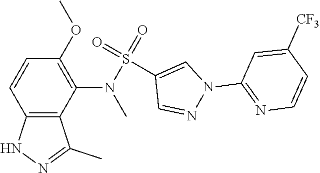 [SMILES: COc1ccc2[nH]nc(C)c2c1N(C)S(=O)(=O)c1cnn(-c2cc(C(F)(F)F)ccn2)c1] |
| I-83 | 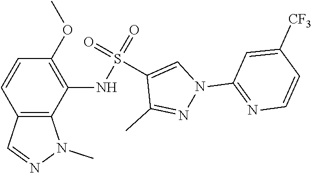 [SMILES: COc1ccc2cnn(C)c2c1NS(=O)(=O)c1cn(-c2cc(C(F)(F)F)ccn2)nc1C] |
| I-84 | 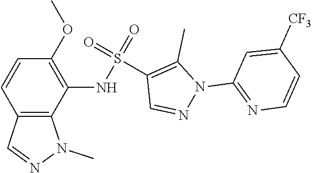 [SMILES: COc1ccc2cnn(C)c2c1NS(=O)(=O)c1cnn(-c2cc(C(F)(F)F)ccn2)c1C] |
| I-85 | 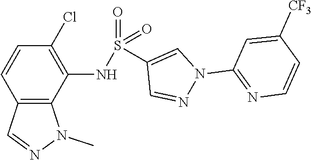 [SMILES: Cn1ncc2ccc(Cl)c(NS(=O)(=O)c3cnn(-c4cc(C(F)(F)F)ccn4)c3)c21] |
| I-86 | 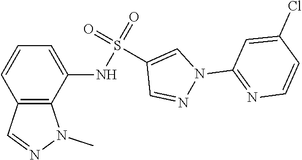 [SMILES: Cn1ncc2cccc(NS(=O)(=O)c3cnn(-c4cc(Cl)ccn4)c3)c21] |
| I-87 | 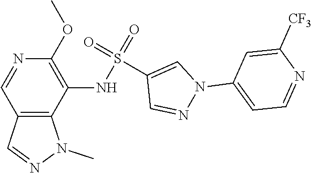 [SMILES: COc1ncc2cnn(C)c2c1NS(=O)(=O)c1cnn(-c2ccnc(C(F)(F)F)c2)c1] |
| I-88 | 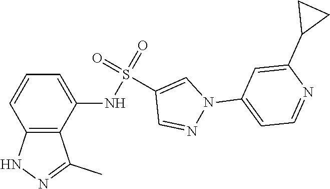 [SMILES: Cc1n[nH]c2cccc(NS(=O)(=O)c3cnn(-c4ccnc(C5CC5)c4)c3)c12] |
| I-89 | 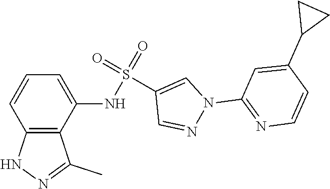 [SMILES: Cc1n[nH]c2cccc(NS(=O)(=O)c3cnn(-c4cc(C5CC5)ccn4)c3)c12] |
| I-90 | 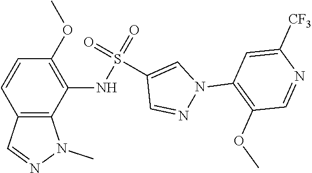 [SMILES: COc1cnc(C(F)(F)F)cc1-n1cc(S(=O)(=O)Nc2c(OC)ccc3cnn(C)c23)cn1] |
| I-91 | 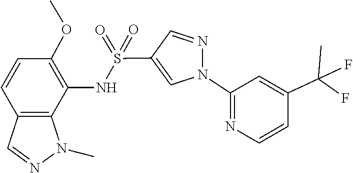 [SMILES: COc1ccc2cnn(C)c2c1NS(=O)(=O)c1cnn(-c2cc(C(C)(F)F)ccn2)c1] |
| I-92 | 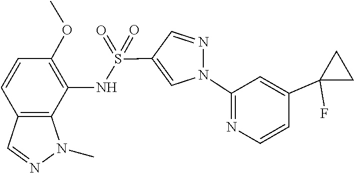 [SMILES: COc1ccc2cnn(C)c2c1NS(=O)(=O)c1cnn(-c2cc(C3(F)CC3)ccn2)c1] |
| I-93 | 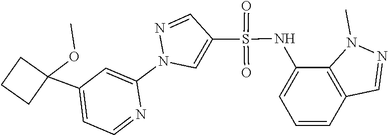 [SMILES: COC1(c2ccnc(-n3cc(S(=O)(=O)Nc4cccc5cnn(C)c45)cn3)c2)CCC1] |
| I-94 | 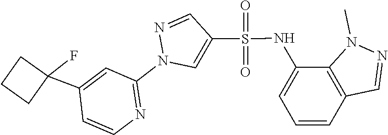 [SMILES: Cn1ncc2cccc(NS(=O)(=O)c3cnn(-c4cc(C5(F)CCC5)ccn4)c3)c21] |
| I-95 | 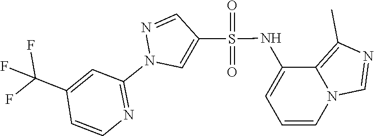 [SMILES: Cc1ncn2cccc(NS(=O)(=O)c3cnn(-c4cc(C(F)(F)F)ccn4)c3)c12] |
| I-96 | 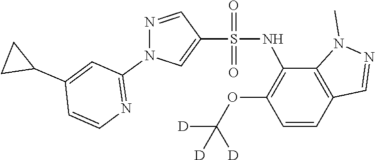 [SMILES: [2H]C([2H])([2H])Oc1ccc2cnn(C)c2c1NS(=O)(=O)c1cnn(-c2cc(C3CC3)ccn2)c1] |
| I-97 | 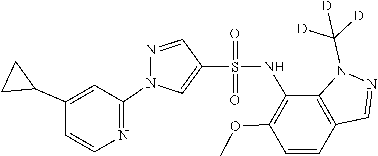 [SMILES: [2H]C([2H])([2H])n1ncc2ccc(OC)c(NS(=O)(=O)c3cnn(-c4cc(C5CC5)ccn4)c3)c21] |
| I-98 | 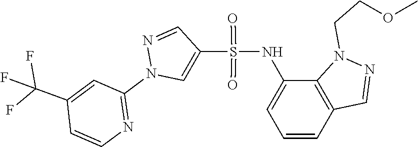 [SMILES: COCCn1ncc2cccc(NS(=O)(=O)c3cnn(-c4cc(C(F)(F)F)ccn4)c3)c21] |
| I-99 | 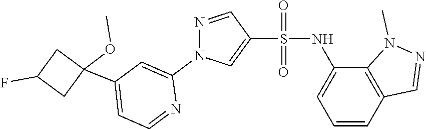 [SMILES: COC1(c2ccnc(-n3cc(S(=O)(=O)Nc4cccc5cnn(C)c45)cn3)c2)CC(F)C1] |
| I-100 | 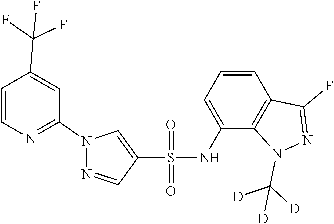 [SMILES: [2H]C([2H])([2H])n1nc(F)c2cccc(NS(=O)(=O)c3cnn(-c4cc(C(F)(F)F)ccn4)c3)c21] |
| I-101 | 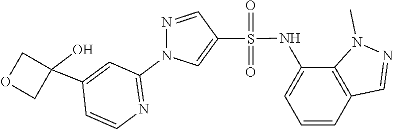 [SMILES: Cn1ncc2cccc(NS(=O)(=O)c3cnn(-c4cc(C5(O)COC5)ccn4)c3)c21] |
| I-102 | 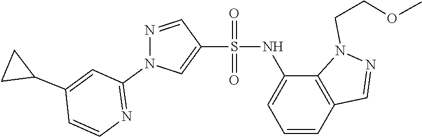 [SMILES: COCCn1ncc2cccc(NS(=O)(=O)c3cnn(-c4cc(C5CC5)ccn4)c3)c21] |
| I-103 | 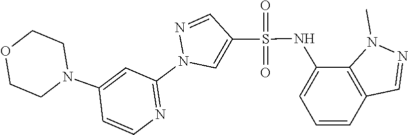 [SMILES: Cn1ncc2cccc(NS(=O)(=O)c3cnn(-c4cc(N5CCOCC5)ccn4)c3)c21] |
| I-104 | 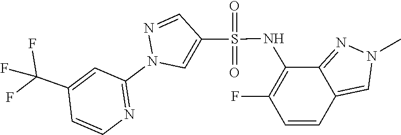 [SMILES: Cn1cc2ccc(F)c(NS(=O)(=O)c3cnn(-c4cc(C(F)(F)F)ccn4)c3)c2n1] |
| I-105 | 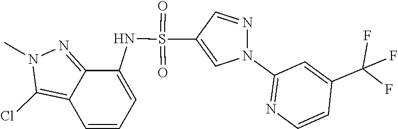 [SMILES: Cn1nc2c(NS(=O)(=O)c3cnn(-c4cc(C(F)(F)F)ccn4)c3)cccc2c1Cl] |
| I-106 | 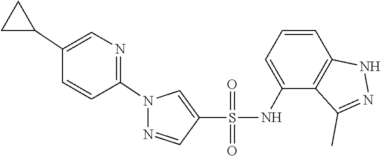 [SMILES: Cc1n[nH]c2cccc(NS(=O)(=O)c3cnn(-c4ccc(C5CC5)cn4)c3)c12] |
| I-107 | 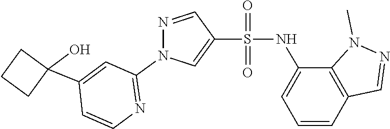 [SMILES: Cn1ncc2cccc(NS(=O)(=O)c3cnn(-c4cc(C5(O)CCC5)ccn4)c3)c21] |
| I-108 | 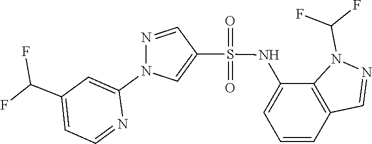 [SMILES: O=S(=O)(Nc1cccc2cnn(C(F)F)c12)c1cnn(-c2cc(C(F)F)ccn2)c1] |
| I-109 | 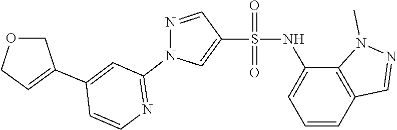 [SMILES: Cn1ncc2cccc(NS(=O)(=O)c3cnn(-c4cc(C5=CCOC5)ccn4)c3)c21] |
| I-110 | 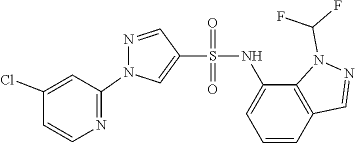 [SMILES: O=S(=O)(Nc1cccc2cnn(C(F)F)c12)c1cnn(-c2cc(Cl)ccn2)c1] |
| I-111 | 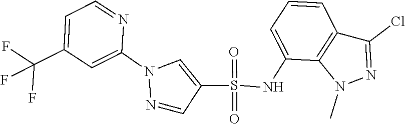 [SMILES: Cn1nc(Cl)c2cccc(NS(=O)(=O)c3cnn(-c4cc(C(F)(F)F)ccn4)c3)c21] |
| I-112 | 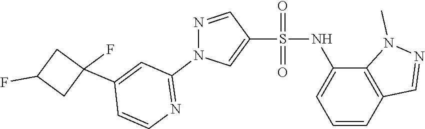 [SMILES: Cn1ncc2cccc(NS(=O)(=O)c3cnn(-c4cc(C5(F)CC(F)C5)ccn4)c3)c21] |
| I-113 | 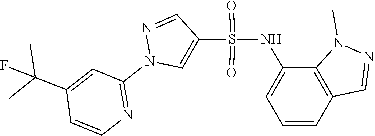 [SMILES: Cn1ncc2cccc(NS(=O)(=O)c3cnn(-c4cc(C(C)(C)F)ccn4)c3)c21] |
| I-114 | 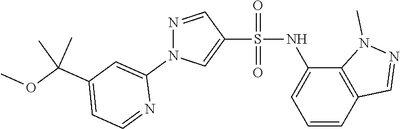 [SMILES: COC(C)(C)c1ccnc(-n2cc(S(=O)(=O)Nc3cccc4cnn(C)c34)cn2)c1] |
| I-115 | 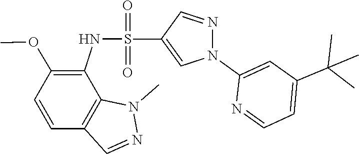 [SMILES: COc1ccc2cnn(C)c2c1NS(=O)(=O)c1cnn(-c2cc(C(C)(C)C)ccn2)c1] |
| I-116 | 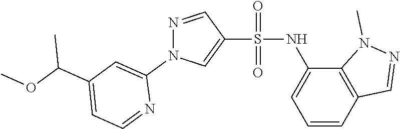 [SMILES: COC(C)c1ccnc(-n2cc(S(=O)(=O)Nc3cccc4cnn(C)c34)cn2)c1] |
| I-117 | 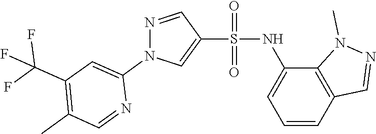 [SMILES: Cc1cnc(-n2cc(S(=O)(=O)Nc3cccc4cnn(C)c34)cn2)cc1C(F)(F)F] |
| I-118 | 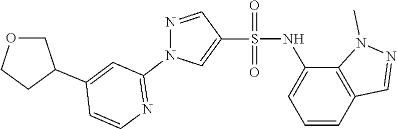 [SMILES: Cn1ncc2cccc(NS(=O)(=O)c3cnn(-c4cc(C5CCOC5)ccn4)c3)c21] |
| I-119 | 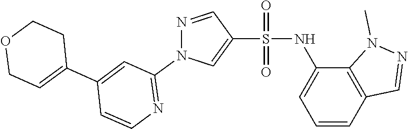 [SMILES: Cn1ncc2cccc(NS(=O)(=O)c3cnn(-c4cc(C5=CCOCC5)ccn4)c3)c21] |
| I-120 | 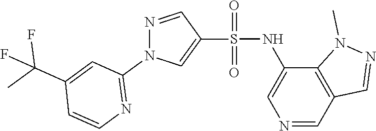 [SMILES: Cn1ncc2cncc(NS(=O)(=O)c3cnn(-c4cc(C(C)(F)F)ccn4)c3)c21] |
| I-121 | 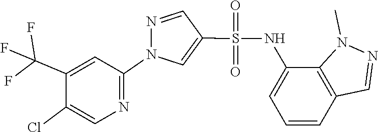 [SMILES: Cn1ncc2cccc(NS(=O)(=O)c3cnn(-c4cc(C(F)(F)F)c(Cl)cn4)c3)c21] |
| I-122 | 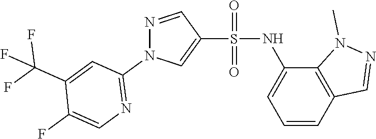 [SMILES: Cn1ncc2cccc(NS(=O)(=O)c3cnn(-c4cc(C(F)(F)F)c(F)cn4)c3)c21] |
| I-123 | 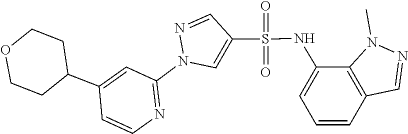 [SMILES: Cn1ncc2cccc(NS(=O)(=O)c3cnn(-c4cc(C5CCOCC5)ccn4)c3)c21] |
| I-124 | 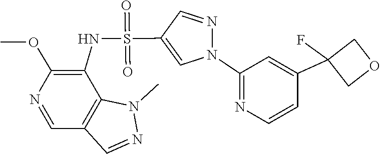 [SMILES: COc1ncc2cnn(C)c2c1NS(=O)(=O)c1cnn(-c2cc(C3(F)COC3)ccn2)c1] |
| I-125 | 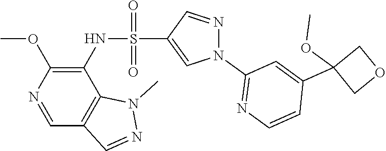 [SMILES: COc1ncc2cnn(C)c2c1NS(=O)(=O)c1cnn(-c2cc(C3(OC)COC3)ccn2)c1] |
| I-126 | 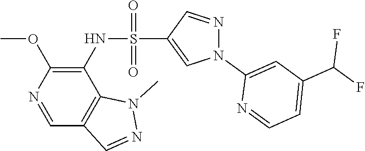 [SMILES: COc1ncc2cnn(C)c2c1NS(=O)(=O)c1cnn(-c2cc(C(F)F)ccn2)c1] |
| I-127 | 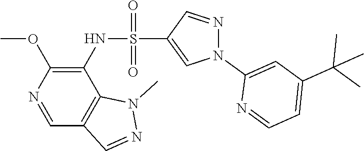 [SMILES: COc1ncc2cnn(C)c2c1NS(=O)(=O)c1cnn(-c2cc(C(C)(C)C)ccn2)c1] |
| I-128 | 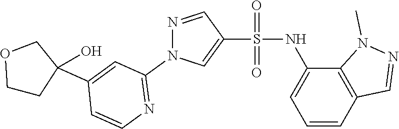 [SMILES: Cn1ncc2cccc(NS(=O)(=O)c3cnn(-c4cc(C5(O)CCOC5)ccn4)c3)c21] |
| I-129 | 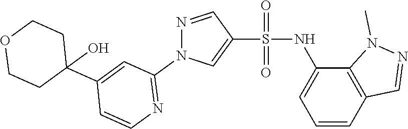 [SMILES: Cn1ncc2cccc(NS(=O)(=O)c3cnn(-c4cc(C5(O)CCOCC5)ccn4)c3)c21] |
| I-130 | 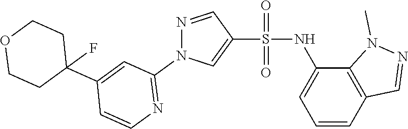 [SMILES: Cn1ncc2cccc(NS(=O)(=O)c3cnn(-c4cc(C5(F)CCOCC5)ccn4)c3)c21] |
| I-131 | 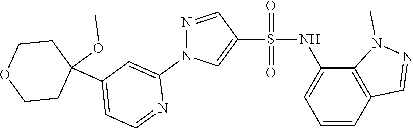 [SMILES: COC1(c2ccnc(-n3cc(S(=O)(=O)Nc4cccc5cnn(C)c45)cn3)c2)CCOCC1] |
| I-132 | 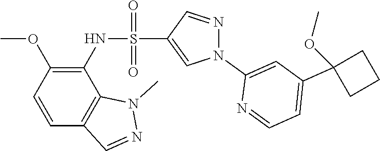 [SMILES: COc1ccc2cnn(C)c2c1NS(=O)(=O)c1cnn(-c2cc(C3(OC)CCC3)ccn2)c1] |
| I-133 | 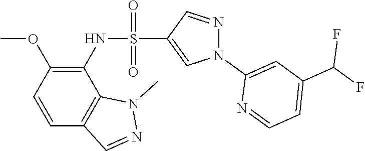 [SMILES: COc1ccc2cnn(C)c2c1NS(=O)(=O)c1cnn(-c2cc(C(F)F)ccn2)c1] |
| I-134 | 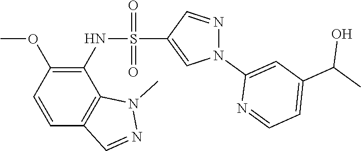 [SMILES: COc1ccc2cnn(C)c2c1NS(=O)(=O)c1cnn(-c2cc(C(C)O)ccn2)c1] |
| I-135 | 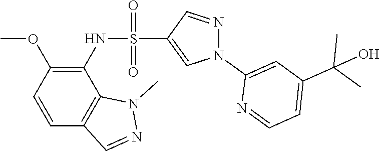 [SMILES: COc1ccc2cnn(C)c2c1NS(=O)(=O)c1cnn(-c2cc(C(C)(C)O)ccn2)c1] |
| I-136 | 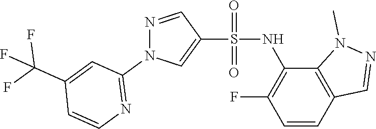 [SMILES: Cn1ncc2ccc(F)c(NS(=O)(=O)c3cnn(-c4cc(C(F)(F)F)ccn4)c3)c21] |
| I-137 | 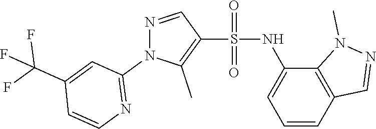 [SMILES: Cc1c(S(=O)(=O)Nc2cccc3cnn(C)c23)cnn1-c1cc(C(F)(F)F)ccn1] |
| I-138 | 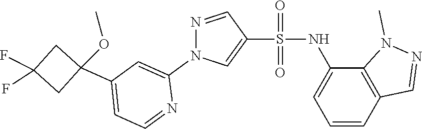 [SMILES: COC1(c2ccnc(-n3cc(S(=O)(=O)Nc4cccc5cnn(C)c45)cn3)c2)CC(F)(F)C1] |
| I-139 | 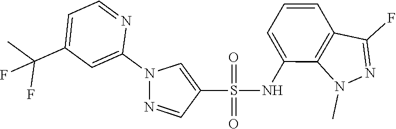 [SMILES: Cn1nc(F)c2cccc(NS(=O)(=O)c3cnn(-c4cc(C(C)(F)F)ccn4)c3)c21] |
| I-140 | 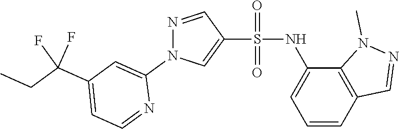 [SMILES: CCC(F)(F)c1ccnc(-n2cc(S(=O)(=O)Nc3cccc4cnn(C)c34)cn2)c1] |
| I-141 | 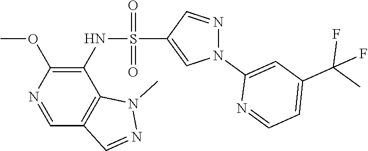 [SMILES: COc1ncc2cnn(C)c2c1NS(=O)(=O)c1cnn(-c2cc(C(C)(F)F)ccn2)c1] |
| I-142 | 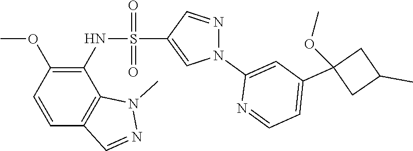 |
| I-143 | 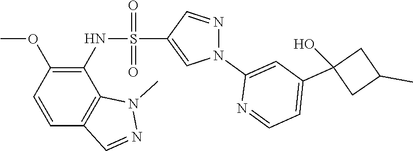 |
| I-144 | 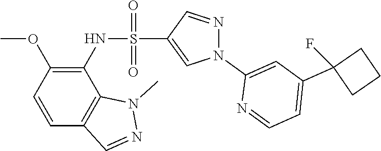 [SMILES: COc1ccc2cnn(C)c2c1NS(=O)(=O)c1cnn(-c2cc(C3(F)CCC3)ccn2)c1] |
| I-145 | 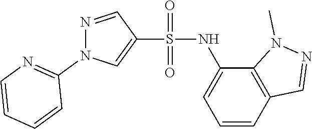 [SMILES: Cn1ncc2cccc(NS(=O)(=O)c3cnn(-c4ccccn4)c3)c21] |
| I-146 | 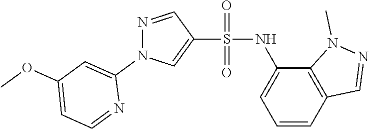 [SMILES: COc1ccnc(-n2cc(S(=O)(=O)Nc3cccc4cnn(C)c34)cn2)c1] |
| I-147 | 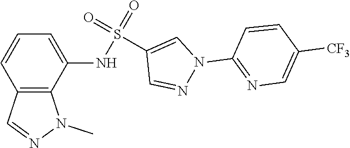 [SMILES: Cn1ncc2cccc(NS(=O)(=O)c3cnn(-c4ccc(C(F)(F)F)cn4)c3)c21] |
| I-148 | 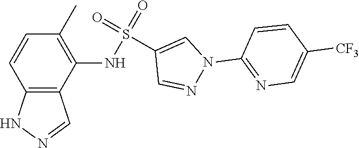 [SMILES: Cc1ccc2[nH]ncc2c1NS(=O)(=O)c1cnn(-c2ccc(C(F)(F)F)cn2)c1] |
| I-149 | 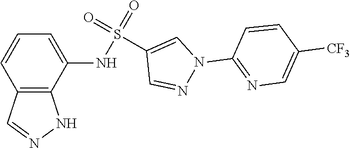 [SMILES: O=S(=O)(Nc1cccc2cn[nH]c12)c1cnn(-c2ccc(C(F)(F)F)cn2)c1] |
| I-150 | 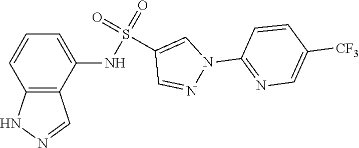 [SMILES: O=S(=O)(Nc1cccc2[nH]ncc12)c1cnn(-c2ccc(C(F)(F)F)cn2)c1] |
| I-151 | 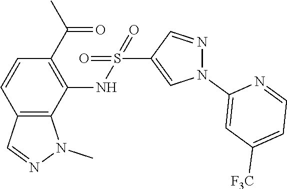 [SMILES: CC(=O)c1ccc2cnn(C)c2c1NS(=O)(=O)c1cnn(-c2cc(C(F)(F)F)ccn2)c1] |
| I-152 | 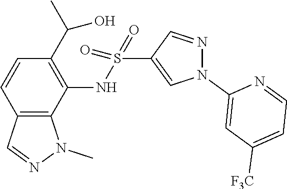 [SMILES: CC(O)c1ccc2cnn(C)c2c1NS(=O)(=O)c1cnn(-c2cc(C(F)(F)F)ccn2)c1] |
| I-153 | 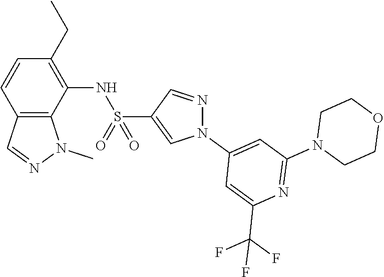 [SMILES: CCc1ccc2cnn(C)c2c1NS(=O)(=O)c1cnn(-c2cc(N3CCOCC3)nc(C(F)(F)F)c2)c1] |
| I-154 | 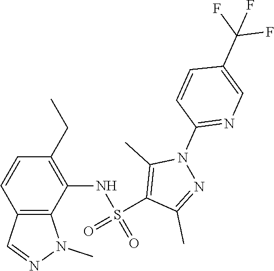 [SMILES: CCc1ccc2cnn(C)c2c1NS(=O)(=O)c1c(C)nn(-c2ccc(C(F)(F)F)cn2)c1C] |
| I-155 | 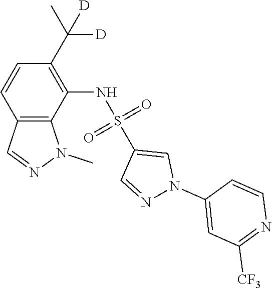 |
| I-156 | 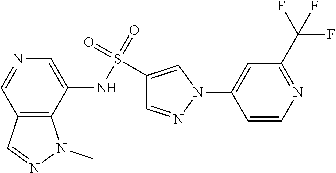 [SMILES: Cn1ncc2cncc(NS(=O)(=O)c3cnn(-c4ccnc(C(F)(F)F)c4)c3)c21] |
| I-157 | 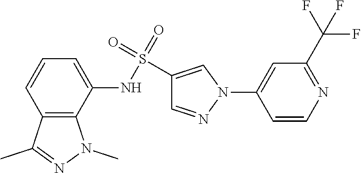 [SMILES: Cc1nn(C)c2c(NS(=O)(=O)c3cnn(-c4ccnc(C(F)(F)F)c4)c3)cccc12] |
| I-158 | 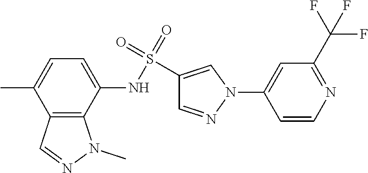 [SMILES: Cc1ccc(NS(=O)(=O)c2cnn(-c3ccnc(C(F)(F)F)c3)c2)c2c1cnn2C] |
| I-159 | 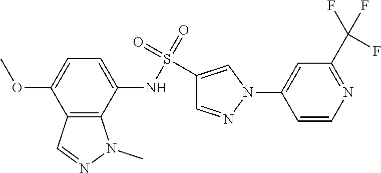 [SMILES: COc1ccc(NS(=O)(=O)c2cnn(-c3ccnc(C(F)(F)F)c3)c2)c2c1cnn2C] |
| I-160 | 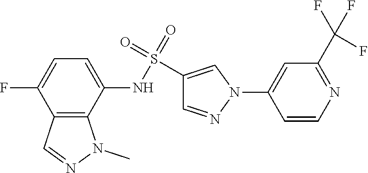 [SMILES: Cn1ncc2c(F)ccc(NS(=O)(=O)c3cnn(-c4ccnc(C(F)(F)F)c4)c3)c21] |
| I-161 | 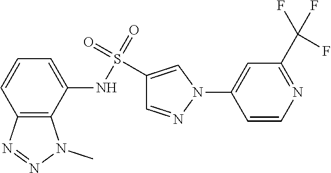 [SMILES: Cn1nnc2cccc(NS(=O)(=O)c3cnn(-c4ccnc(C(F)(F)F)c4)c3)c21] |
| I-162 | 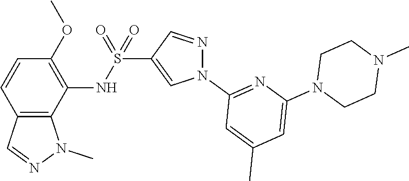 [SMILES: COc1ccc2cnn(C)c2c1NS(=O)(=O)c1cnn(-c2cc(C)cc(N3CCN(C)CC3)n2)c1] |
| I-163 | 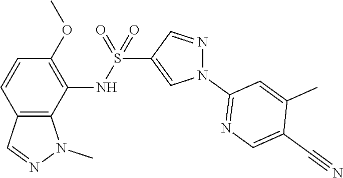 [SMILES: COc1ccc2cnn(C)c2c1NS(=O)(=O)c1cnn(-c2cc(C)c(C#N)cn2)c1] |
| I-164 | 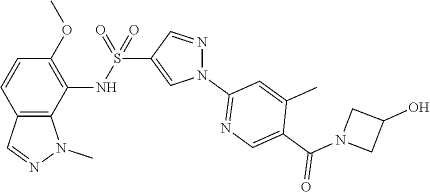 [SMILES: COc1ccc2cnn(C)c2c1NS(=O)(=O)c1cnn(-c2cc(C)c(C(=O)N3CC(O)C3)cn2)c1] |
| I-165 | 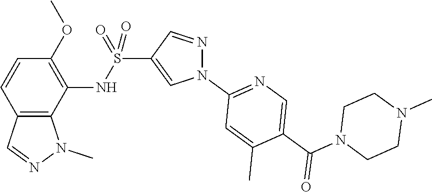 [SMILES: COc1ccc2cnn(C)c2c1NS(=O)(=O)c1cnn(-c2cc(C)c(C(=O)N3CCN(C)CC3)cn2)c1] |
| I-166 | 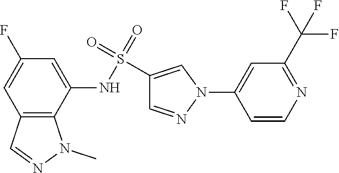 [SMILES: Cn1ncc2cc(F)cc(NS(=O)(=O)c3cnn(-c4ccnc(C(F)(F)F)c4)c3)c21] |
| I-167 | 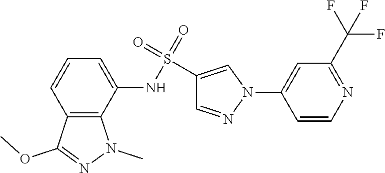 [SMILES: COc1nn(C)c2c(NS(=O)(=O)c3cnn(-c4ccnc(C(F)(F)F)c4)c3)cccc12] |
| I-168 | 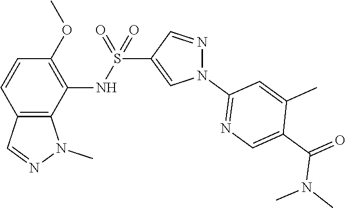 [SMILES: COc1ccc2cnn(C)c2c1NS(=O)(=O)c1cnn(-c2cc(C)c(C(=O)N(C)C)cn2)c1] |
| I-169 | 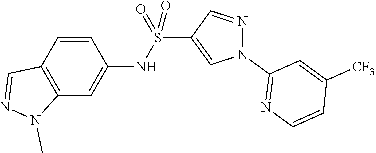 [SMILES: Cn1ncc2ccc(NS(=O)(=O)c3cnn(-c4cc(C(F)(F)F)ccn4)c3)cc21] |
| I-170 | 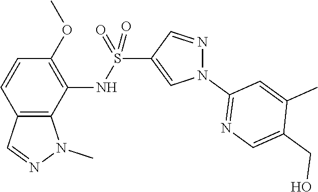 [SMILES: COc1ccc2cnn(C)c2c1NS(=O)(=O)c1cnn(-c2cc(C)c(CO)cn2)c1] |
| I-171 | 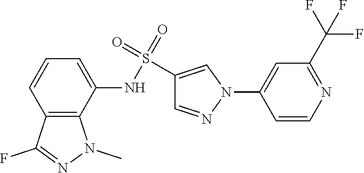 [SMILES: Cn1nc(F)c2cccc(NS(=O)(=O)c3cnn(-c4ccnc(C(F)(F)F)c4)c3)c21] |
| I-172 | 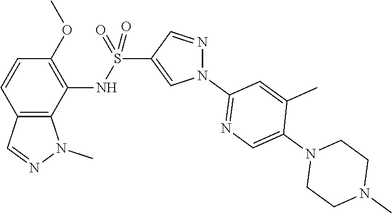 [SMILES: COc1ccc2cnn(C)c2c1NS(=O)(=O)c1cnn(-c2cc(C)c(N3CCN(C)CC3)cn2)c1] |
| I-173 | 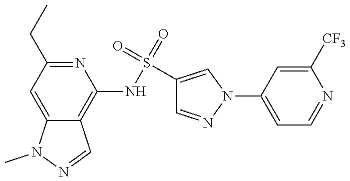 [SMILES: CCc1cc2c(cnn2C)c(NS(=O)(=O)c2cnn(-c3ccnc(C(F)(F)F)c3)c2)n1] |
| I-174 | 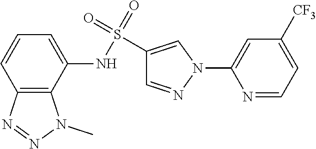 [SMILES: Cn1nnc2cccc(NS(=O)(=O)c3cnn(-c4cc(C(F)(F)F)ccn4)c3)c21] |
| I-175 | 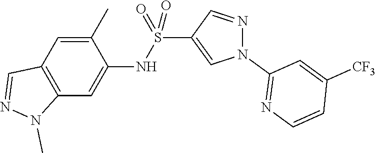 [SMILES: Cc1cc2cnn(C)c2cc1NS(=O)(=O)c1cnn(-c2cc(C(F)(F)F)ccn2)c1] |
| I-176 | 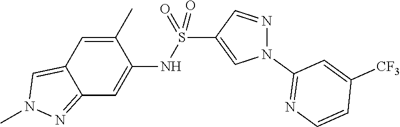 [SMILES: Cc1cc2cn(C)nc2cc1NS(=O)(=O)c1cnn(-c2cc(C(F)(F)F)ccn2)c1] |
| I-178 | 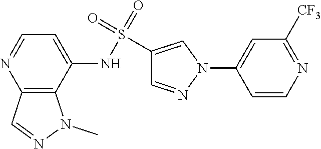 [SMILES: Cn1ncc2nccc(NS(=O)(=O)c3cnn(-c4ccnc(C(F)(F)F)c4)c3)c21] |
| I-179 | 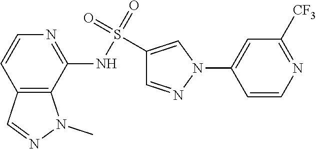 [SMILES: Cn1ncc2ccnc(NS(=O)(=O)c3cnn(-c4ccnc(C(F)(F)F)c4)c3)c21] |
| I-180 | 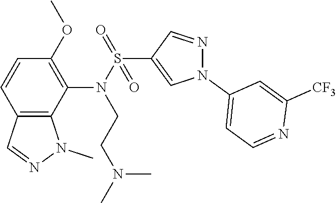 [SMILES: COc1ccc2cnn(C)c2c1N(CCN(C)C)S(=O)(=O)c1cnn(-c2ccnc(C(F)(F)F)c2)c1] |
| I-181 | 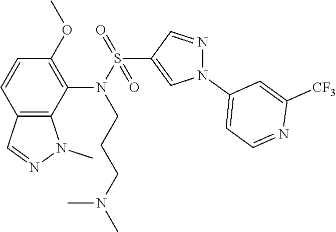 [SMILES: COc1ccc2cnn(C)c2c1N(CCCN(C)C)S(=O)(=O)c1cnn(-c2ccnc(C(F)(F)F)c2)c1] |
| I-182 | 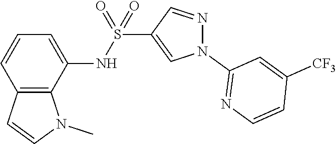 [SMILES: Cn1ccc2cccc(NS(=O)(=O)c3cnn(-c4cc(C(F)(F)F)ccn4)c3)c21] |
| I-183 | 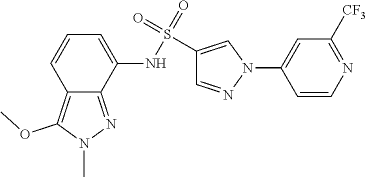 [SMILES: COc1c2cccc(NS(=O)(=O)c3cnn(-c4ccnc(C(F)(F)F)c4)c3)c2nn1C] |
| I-184 | 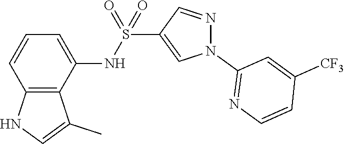 [SMILES: Cc1c[nH]c2cccc(NS(=O)(=O)c3cnn(-c4cc(C(F)(F)F)ccn4)c3)c12] |
| I-185 | 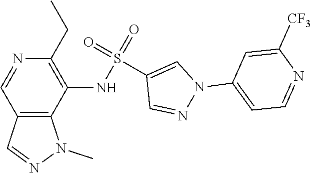 [SMILES: CCc1ncc2cnn(C)c2c1NS(=O)(=O)c1cnn(-c2ccnc(C(F)(F)F)c2)c1] |
| I-186 | 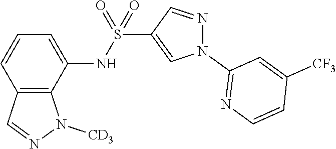 [SMILES: [2H]C([2H])([2H])n1ncc2cccc(NS(=O)(=O)c3cnn(-c4cc(C(F)(F)F)ccn4)c3)c21] |
| I-187 | 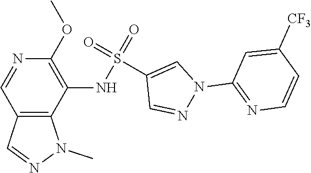 [SMILES: COc1ncc2cnn(C)c2c1NS(=O)(=O)c1cnn(-c2cc(C(F)(F)F)ccn2)c1] |
| I-188 | 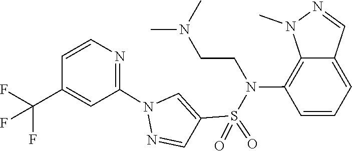 [SMILES: CN(C)CCN(c1cccc2cnn(C)c12)S(=O)(=O)c1cnn(-c2cc(C(F)(F)F)ccn2)c1] |
| I-189 | 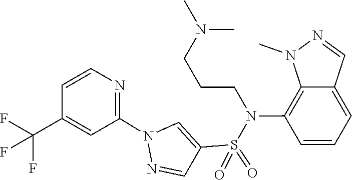 [SMILES: CN(C)CCCN(c1cccc2cnn(C)c12)S(=O)(=O)c1cnn(-c2cc(C(F)(F)F)ccn2)c1] |
| I-190 | 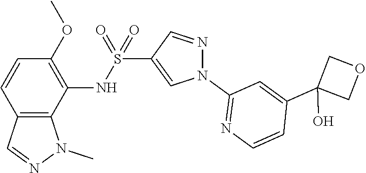 [SMILES: COc1ccc2cnn(C)c2c1NS(=O)(=O)c1cnn(-c2cc(C3(O)COC3)ccn2)c1] |
| I-191 | 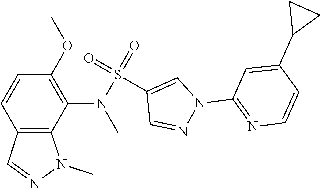 [SMILES: COc1ccc2cnn(C)c2c1N(C)S(=O)(=O)c1cnn(-c2cc(C3CC3)ccn2)c1] |
| I-192 | 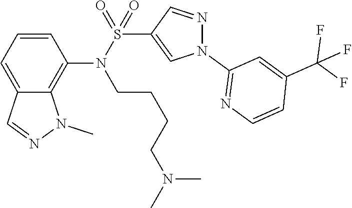 [SMILES: CN(C)CCCCN(c1cccc2cnn(C)c12)S(=O)(=O)c1cnn(-c2cc(C(F)(F)F)ccn2)c1] |
| I-193 | 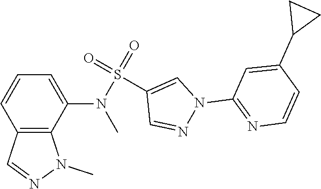 [SMILES: CN(c1cccc2cnn(C)c12)S(=O)(=O)c1cnn(-c2cc(C3CC3)ccn2)c1] |
| I-194 | 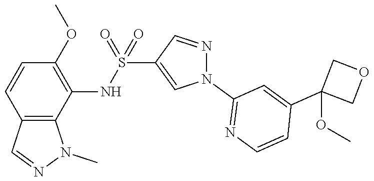 [SMILES: COc1ccc2cnn(C)c2c1NS(=O)(=O)c1cnn(-c2cc(C3(OC)COC3)ccn2)c1] |
| I-195 | 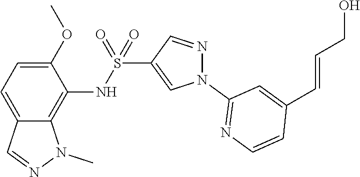 [SMILES: COc1ccc2cnn(C)c2c1NS(=O)(=O)c1cnn(-c2cc(/C=C/CO)ccn2)c1] |
| I-196 | 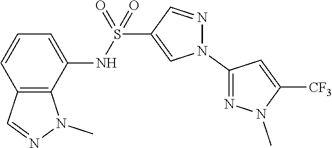 [SMILES: Cn1nc(-n2cc(S(=O)(=O)Nc3cccc4cnn(C)c34)cn2)cc1C(F)(F)F] |
| I-197 | 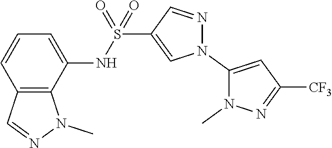 [SMILES: Cn1nc(C(F)(F)F)cc1-n1cc(S(=O)(=O)Nc2cccc3cnn(C)c23)cn1] |
| I-198 | 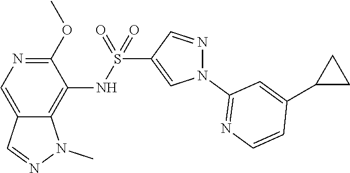 [SMILES: COc1ncc2cnn(C)c2c1NS(=O)(=O)c1cnn(-c2cc(C3CC3)ccn2)c1] |
| I-199 | 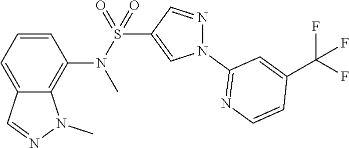 [SMILES: CN(c1cccc2cnn(C)c12)S(=O)(=O)c1cnn(-c2cc(C(F)(F)F)ccn2)c1] |
| I-200 | 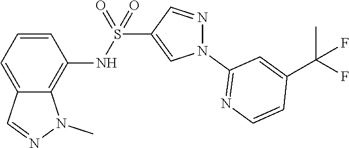 [SMILES: Cn1ncc2cccc(NS(=O)(=O)c3cnn(-c4cc(C(C)(F)F)ccn4)c3)c21] |
| I-201 | 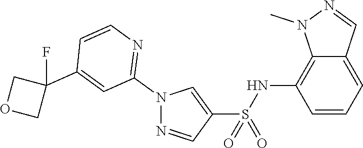 [SMILES: Cn1ncc2cccc(NS(=O)(=O)c3cnn(-c4cc(C5(F)COC5)ccn4)c3)c21] |
| I-202 | 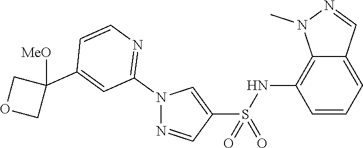 [SMILES: COC1(c2ccnc(-n3cc(S(=O)(=O)Nc4cccc5cnn(C)c45)cn3)c2)COC1] |
| I-203 | 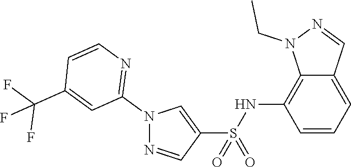 [SMILES: CCn1ncc2cccc(NS(=O)(=O)c3cnn(-c4cc(C(F)(F)F)ccn4)c3)c21] |
| I-204 | 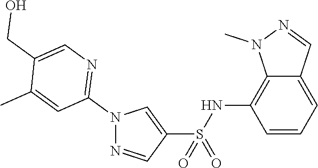 [SMILES: Cc1cc(-n2cc(S(=O)(=O)Nc3cccc4cnn(C)c34)cn2)ncc1CO] |
| I-205 | 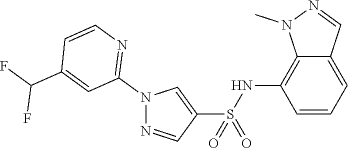 [SMILES: Cn1ncc2cccc(NS(=O)(=O)c3cnn(-c4cc(C(F)F)ccn4)c3)c21] |
| I-206 | 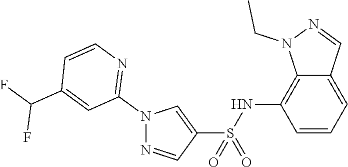 [SMILES: CCn1ncc2cccc(NS(=O)(=O)c3cnn(-c4cc(C(F)F)ccn4)c3)c21] |
| I-207 | 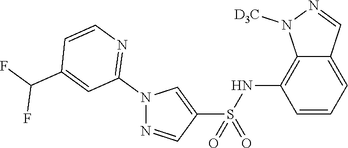 [SMILES: [2H]C([2H])([2H])n1ncc2cccc(NS(=O)(=O)c3cnn(-c4cc(C(F)F)ccn4)c3)c21] |
| I-208 | 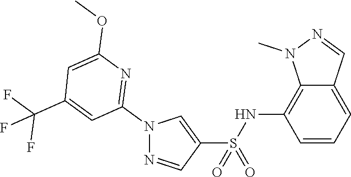 [SMILES: COc1cc(C(F)(F)F)cc(-n2cc(S(=O)(=O)Nc3cccc4cnn(C)c34)cn2)n1] |
| I-209 | 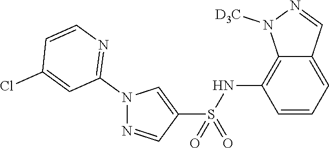 [SMILES: [2H]C([2H])([2H])n1ncc2cccc(NS(=O)(=O)c3cnn(-c4cc(Cl)ccn4)c3)c21] |
| I-210 | 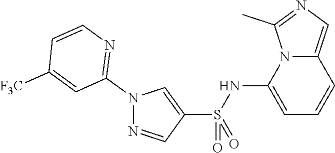 [SMILES: Cc1ncc2cccc(NS(=O)(=O)c3cnn(-c4cc(C(F)(F)F)ccn4)c3)n12] |
| I-211 | 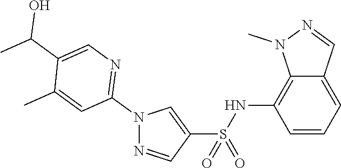 [SMILES: Cc1cc(-n2cc(S(=O)(=O)Nc3cccc4cnn(C)c34)cn2)ncc1C(C)O] |
| I-212 | 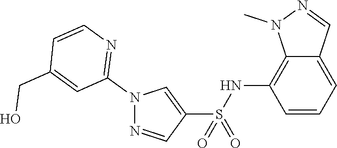 [SMILES: Cn1ncc2cccc(NS(=O)(=O)c3cnn(-c4cc(CO)ccn4)c3)c21] |
| I-213 | 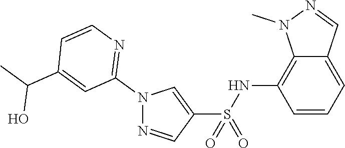 [SMILES: CC(O)c1ccnc(-n2cc(S(=O)(=O)Nc3cccc4cnn(C)c34)cn2)c1] |
| I-214 | 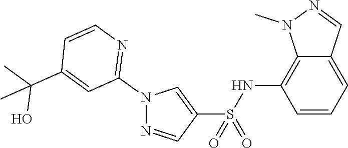 [SMILES: Cn1ncc2cccc(NS(=O)(=O)c3cnn(-c4cc(C(C)(C)O)ccn4)c3)c21] |
| I-215 | 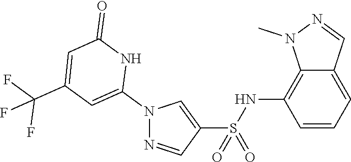 [SMILES: Cn1ncc2cccc(NS(=O)(=O)c3cnn(-c4cc(C(F)(F)F)cc(=O)[nH]4)c3)c21] |
| I-216 | 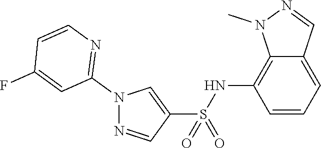 [SMILES: Cn1ncc2cccc(NS(=O)(=O)c3cnn(-c4cc(F)ccn4)c3)c21] |
| I-217 | 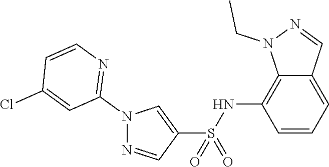 [SMILES: CCn1ncc2cccc(NS(=O)(=O)c3cnn(-c4cc(Cl)ccn4)c3)c21] |
| I-218 | 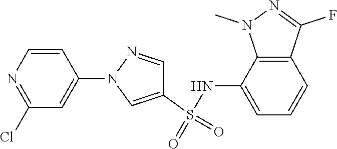 [SMILES: Cn1nc(F)c2cccc(NS(=O)(=O)c3cnn(-c4ccnc(Cl)c4)c3)c21] |
| I-219 | 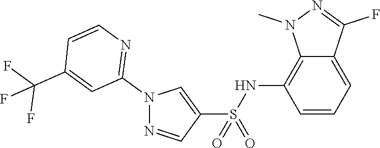 [SMILES: Cn1nc(F)c2cccc(NS(=O)(=O)c3cnn(-c4cc(C(F)(F)F)ccn4)c3)c21] |
| I-220 | 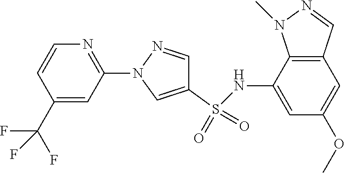 [SMILES: COc1cc(NS(=O)(=O)c2cnn(-c3cc(C(F)(F)F)ccn3)c2)c2c(cnn2C)c1] |
| I-221 | 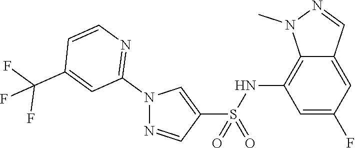 [SMILES: Cn1ncc2cc(F)cc(NS(=O)(=O)c3cnn(-c4cc(C(F)(F)F)ccn4)c3)c21] |
| I-222 | 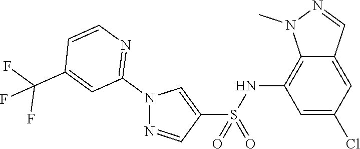 [SMILES: Cn1ncc2cc(Cl)cc(NS(=O)(=O)c3cnn(-c4cc(C(F)(F)F)ccn4)c3)c21] |
| I-223 | 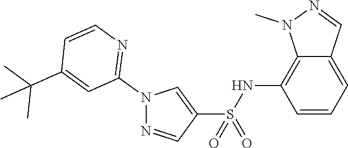 [SMILES: Cn1ncc2cccc(NS(=O)(=O)c3cnn(-c4cc(C(C)(C)C)ccn4)c3)c21] |
| I-224 | 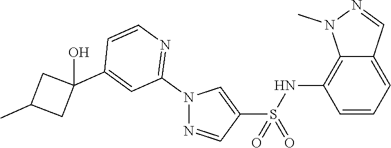 [SMILES: CC1CC(O)(c2ccnc(-n3cc(S(=O)(=O)Nc4cccc5cnn(C)c45)cn3)c2)C1] |
| I-225 | 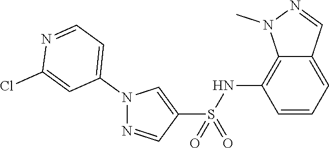 [SMILES: Cn1ncc2cccc(NS(=O)(=O)c3cnn(-c4ccnc(Cl)c4)c3)c21] |
| I-226 | 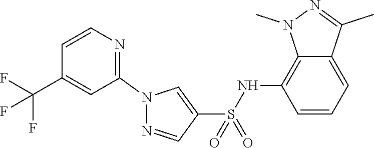 [SMILES: Cc1nn(C)c2c(NS(=O)(=O)c3cnn(-c4cc(C(F)(F)F)ccn4)c3)cccc12] |
| I-227 | 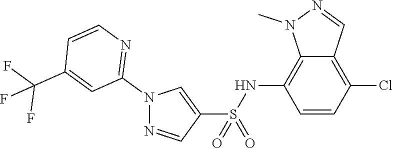 [SMILES: Cn1ncc2c(Cl)ccc(NS(=O)(=O)c3cnn(-c4cc(C(F)(F)F)ccn4)c3)c21] |
| I-228 | 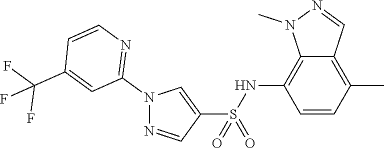 [SMILES: Cc1ccc(NS(=O)(=O)c2cnn(-c3cc(C(F)(F)F)ccn3)c2)c2c1cnn2C] |
| I-229 | 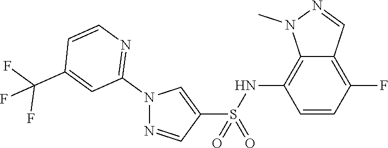 [SMILES: Cn1ncc2c(F)ccc(NS(=O)(=O)c3cnn(-c4cc(C(F)(F)F)ccn4)c3)c21] |
| I-230 | 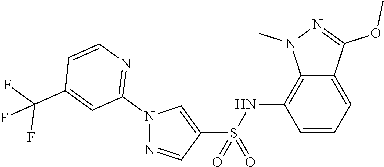 [SMILES: COc1nn(C)c2c(NS(=O)(=O)c3cnn(-c4cc(C(F)(F)F)ccn4)c3)cccc12] |
| I-231 | 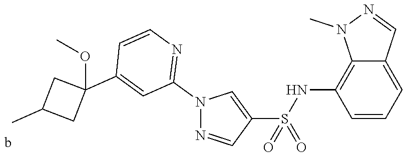 [SMILES: COC1(c2ccnc(-n3cc(S(=O)(=O)Nc4cccc5cnn(C)c45)cn3)c2)CC(C)C1] |
| I-232 | 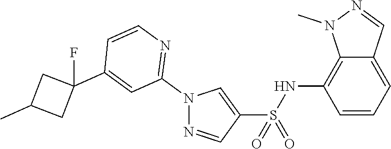 [SMILES: CC1CC(F)(c2ccnc(-n3cc(S(=O)(=O)Nc4cccc5cnn(C)c45)cn3)c2)C1] |
| I-233 | 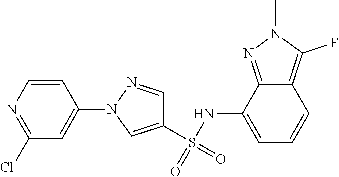 [SMILES: Cn1nc2c(NS(=O)(=O)c3cnn(-c4ccnc(Cl)c4)c3)cccc2c1F] |
| I-234 | 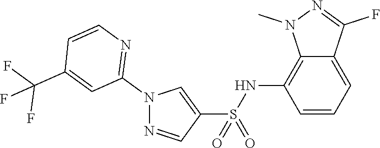 [SMILES: Cn1nc(F)c2cccc(NS(=O)(=O)c3cnn(-c4cc(C(F)(F)F)ccn4)c3)c21] |
| I-235 | 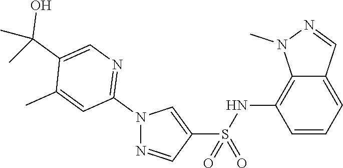 [SMILES: Cc1cc(-n2cc(S(=O)(=O)Nc3cccc4cnn(C)c34)cn2)ncc1C(C)(C)O] |
| I-236 | 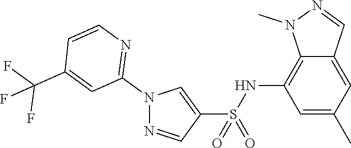 [SMILES: Cc1cc(NS(=O)(=O)c2cnn(-c3cc(C(F)(F)F)ccn3)c2)c2c(cnn2C)c1] |
| I-237 | 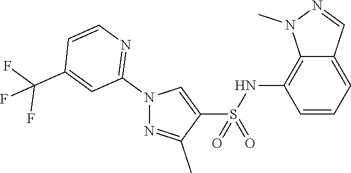 [SMILES: Cc1nn(-c2cc(C(F)(F)F)ccn2)cc1S(=O)(=O)Nc1cccc2cnn(C)c12] |
| I-239 | 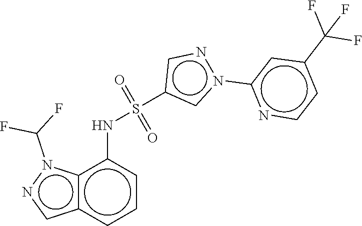 |
| I-240 | 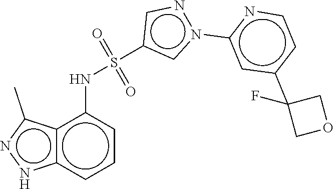 |
| I-241 | 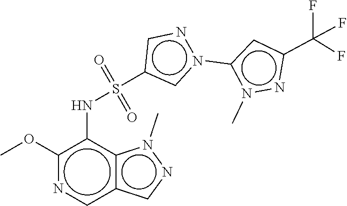 |
| I-242 | 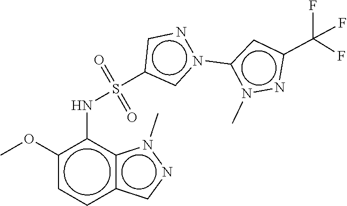 |
| I-243 | 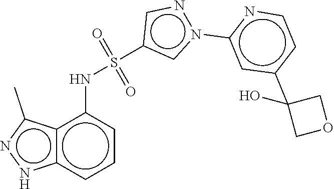 |
| I-244 | 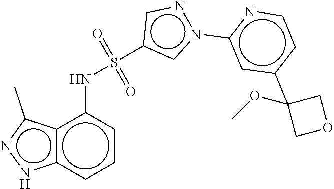 |
| I-245 | 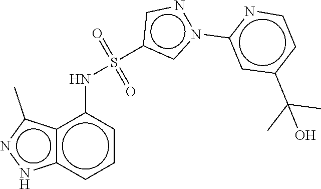 |
| I-246 | 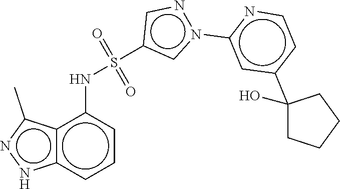 |
| I-247 | 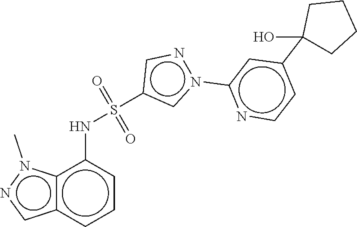 |
| I-248 | 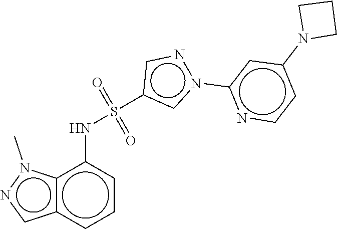 |
| I-249 | 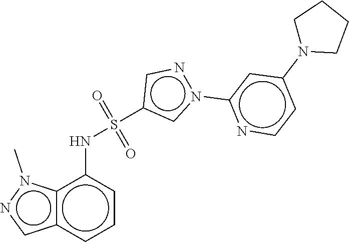 |
| I-250 | 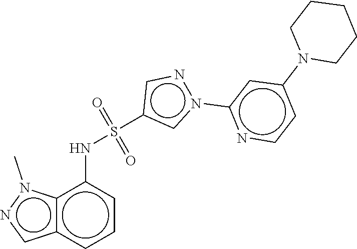 |
| I-251 | 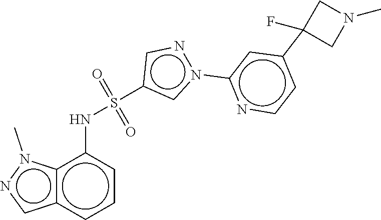 |
| I-252 | 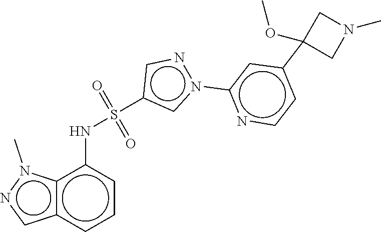 |
| I-253 | 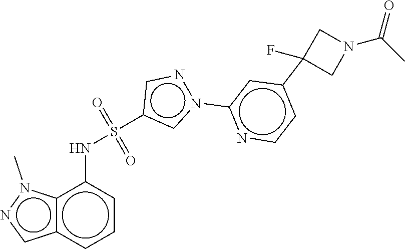 |
| I-254 | 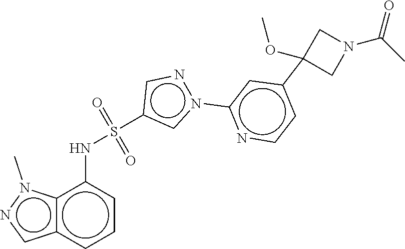 |
| I-255 | 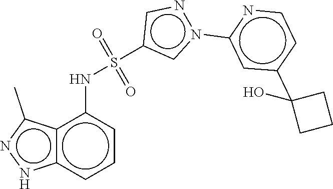 |
| I-256 | 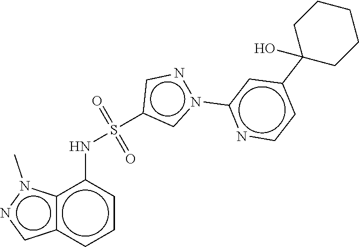 |
| I-257 | 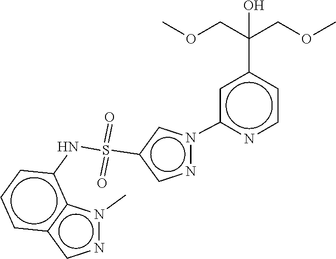 |
| I-258 | 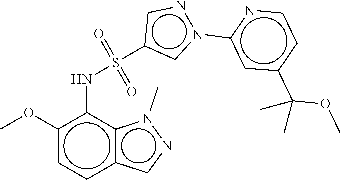 |
| I-259 | 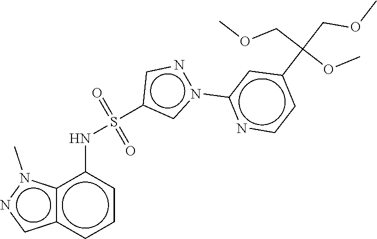 |
| I-260 | 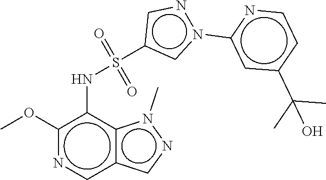 |
| I-261 | 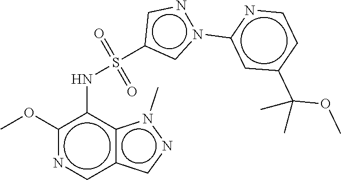 |
| I-262 | 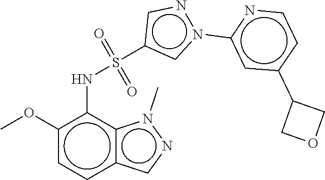 |
| I-263 | 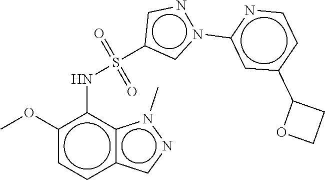 |
| I-264 | 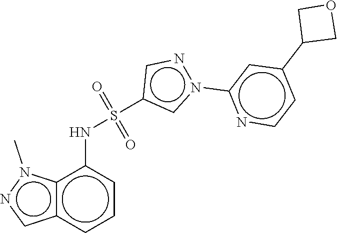 |
| I-265 | 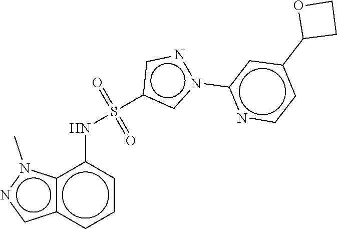 |
| I-266 | 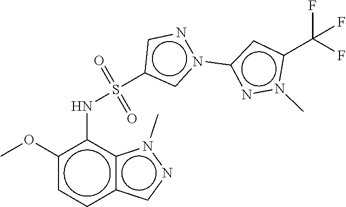 |
| I-267 | 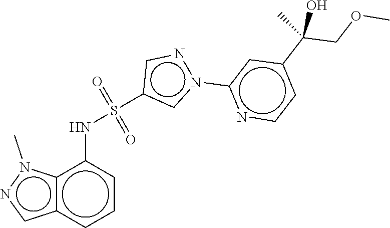 |
| I-268 | 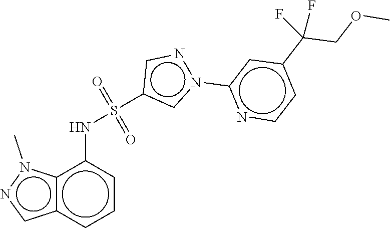 |
| I-269 | 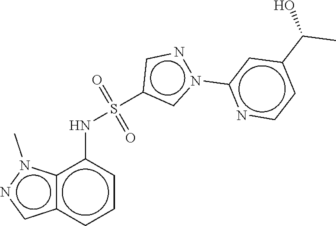 |
| I-270 | 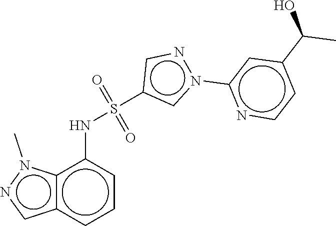 |
| I-271 | 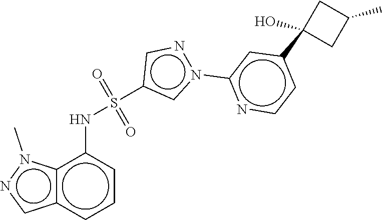 |
| I-272 | 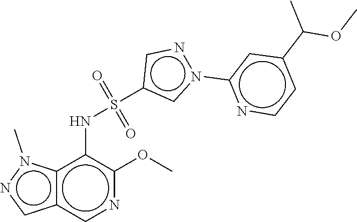 |
| I-273 | 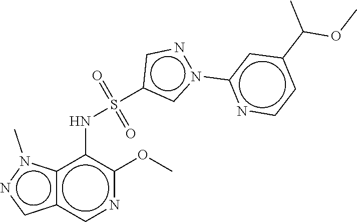 |
| I-274 | 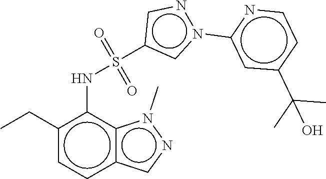 |
| I-275 | 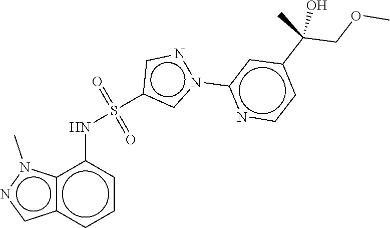 |
| I-276 | 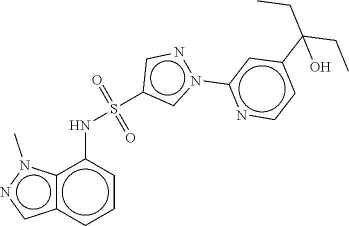 |
| I-277 | 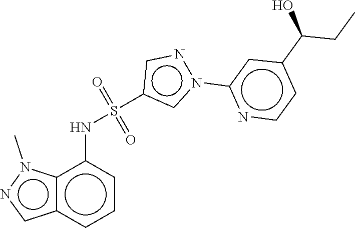 |
| I-278 | 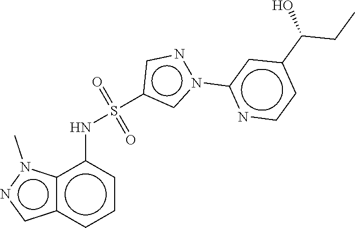 |
| I-279 | 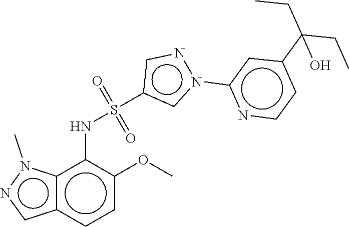 |
| I-280 | 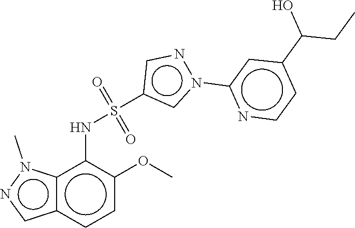 |
| I-281 | 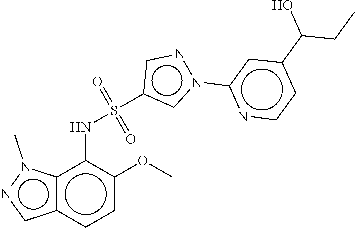 |
| I-282 | 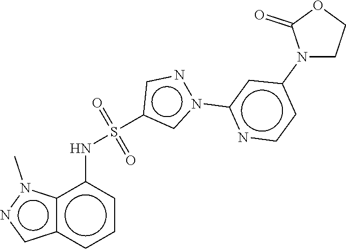 |
| I-283 | 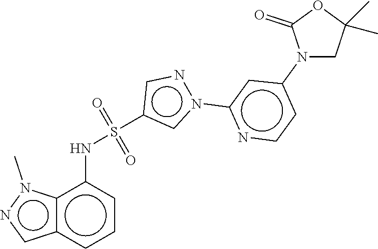 |
| I-284 | 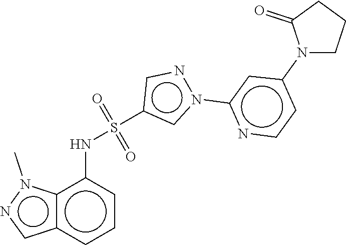 |
| I-285 | 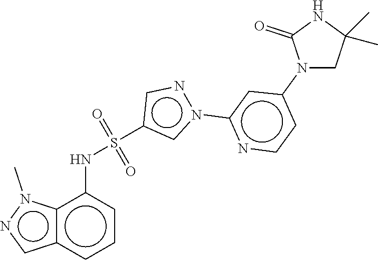 |
| I-286 | 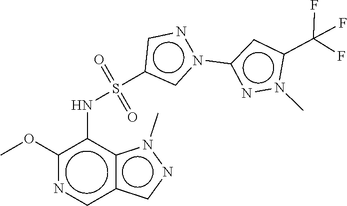 |
| I-287 | 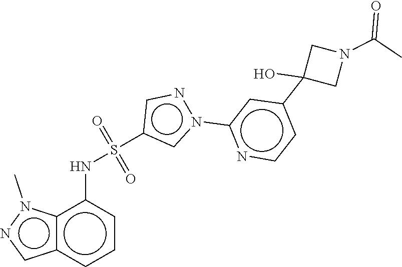 |
| I-288 | 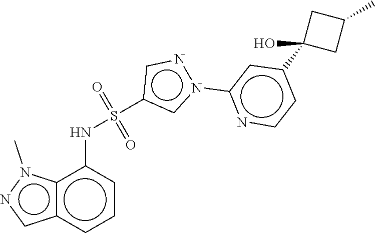 |
| I-289 | 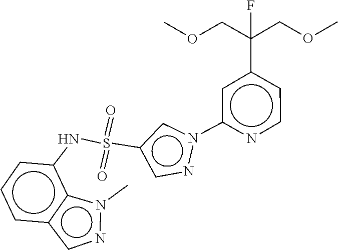 |
| I-290 | 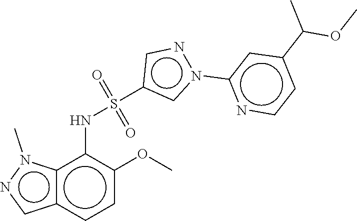 |
| I-291 | 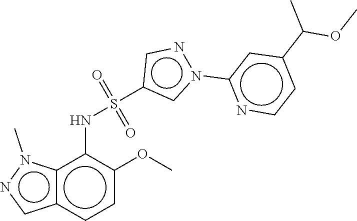 |
| I-292 | 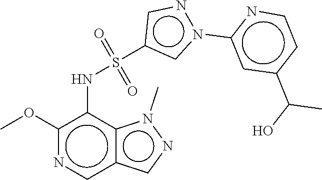 |
| I-293 | 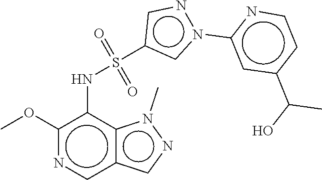 |
| I-294 | 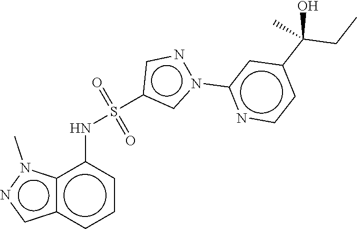 |
| I-295 | 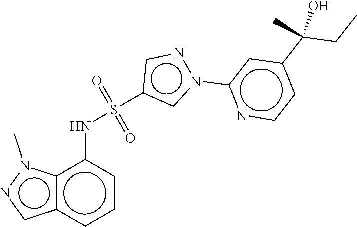 |
| I-296 | 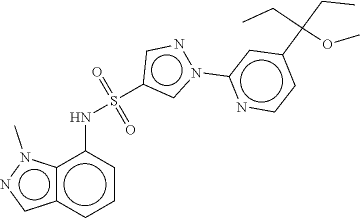 |
| I-297 | 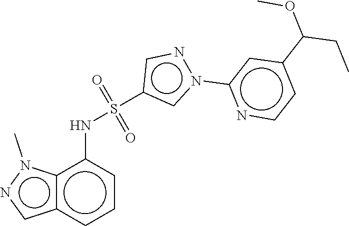 |
| I-298 | 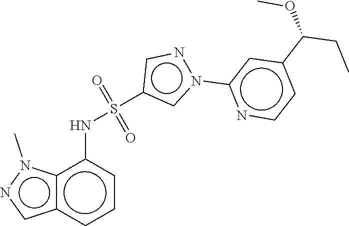 |
| I-299 | 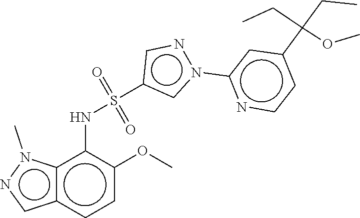 |
| I-300 | 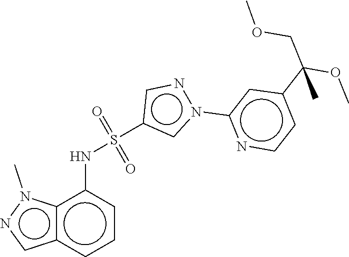 |
| I-301 | 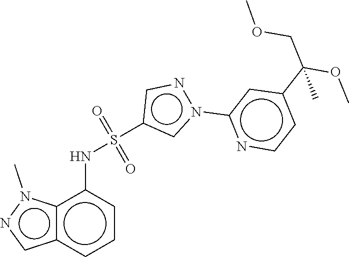 |
| I-302 | 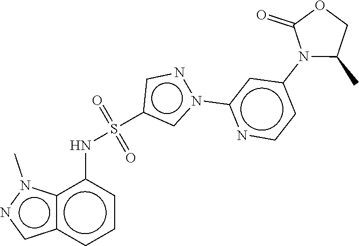 |
| I-303 | 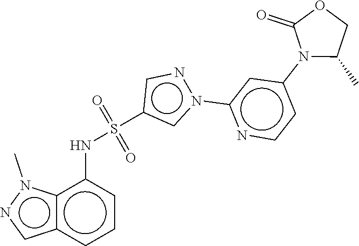 |
| I-304 | 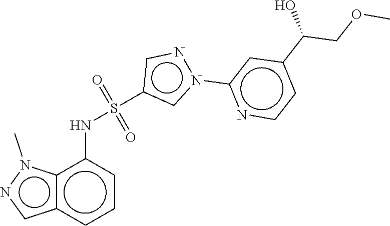 |
| I-305 | 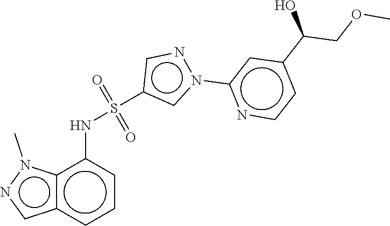 |
| I-306 | 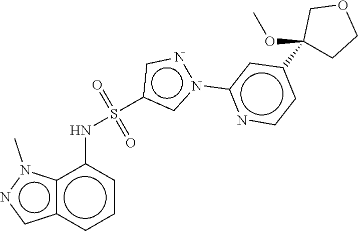 |
| I-307 | 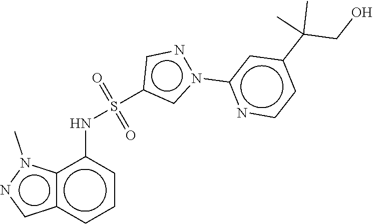 |
| I-308 | 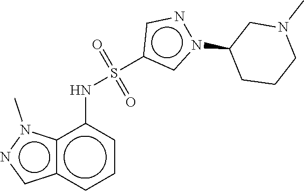 |
| I-309 | 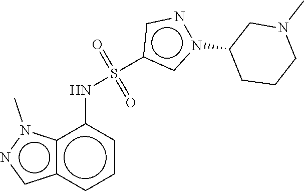 |
| I-310 | 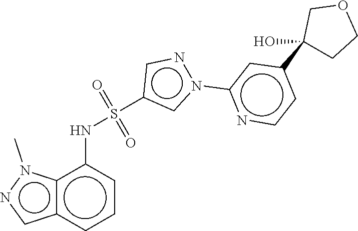 |
| I-311 | 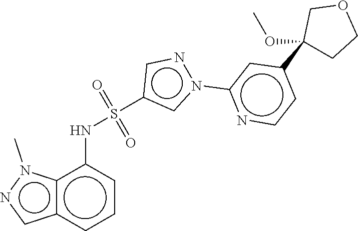 |
| I-312 | 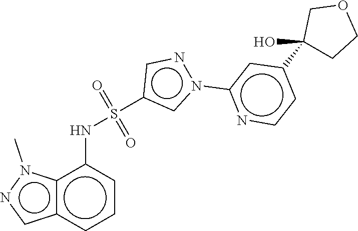 |
| I-313 | 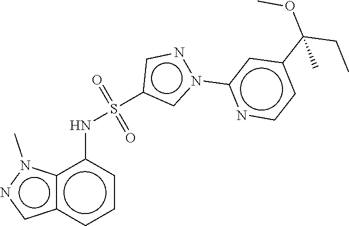 |
| I-314 | 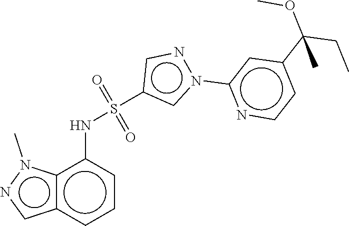 |
| I-315 | 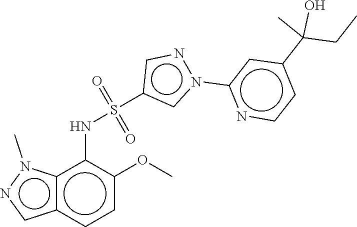 |
| I-316 | 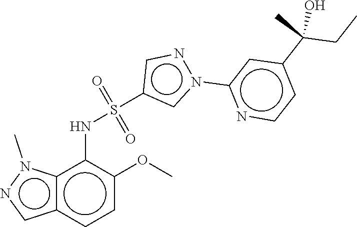 |
| I-317 | 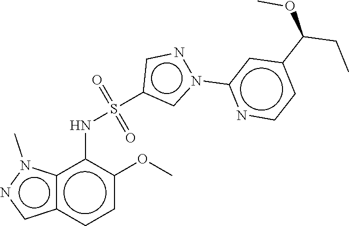 |
| I-318 | 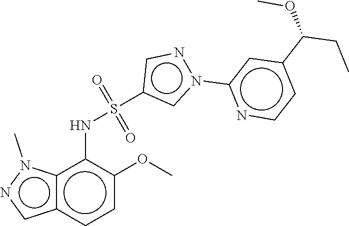 |
| I-319 | 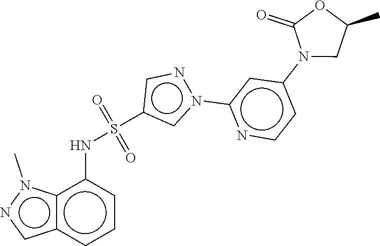 |
| I-320 | 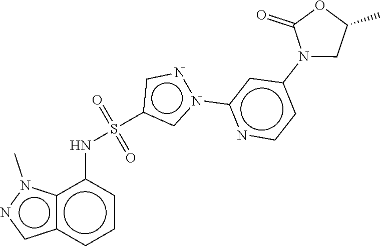 |
| I-321 | 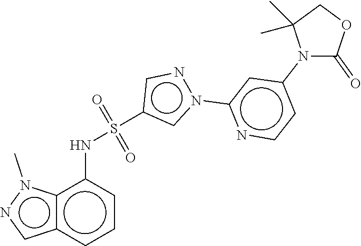 |
| I-322 | 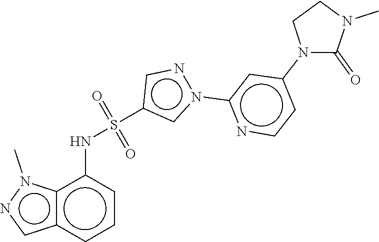 |
| I-323 | 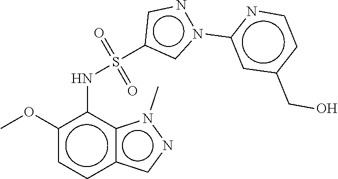 |
| I-324 | 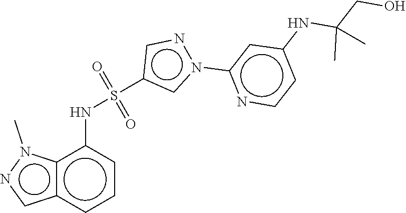 |
| I-325 | 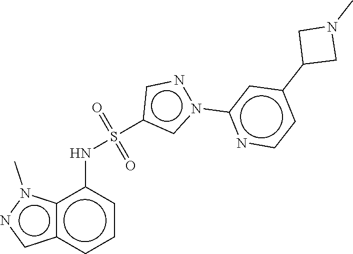 |
| I-326 | 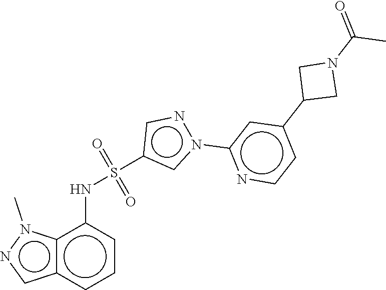 |
| I-327 | 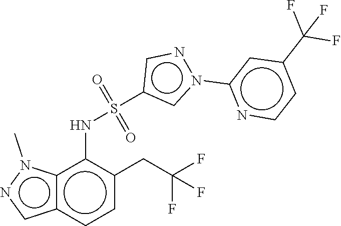 |
| I-328 | 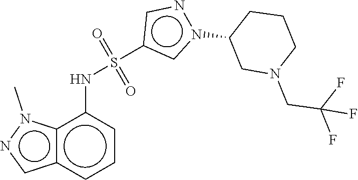 |
| I-329 | 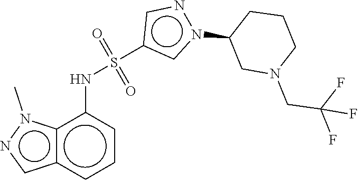 |
| I-330 | 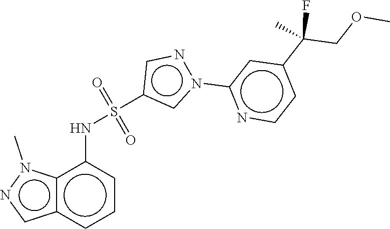 |
| I-331 | 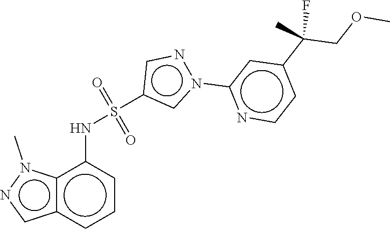 |
| I-332 | 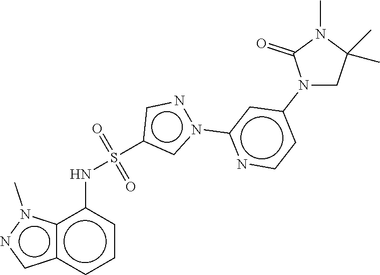 |
| I-333 | 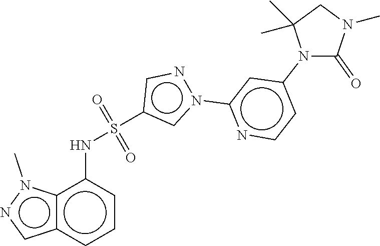 |
| I-334 | 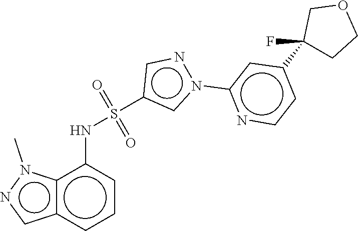 |
| I-335 | 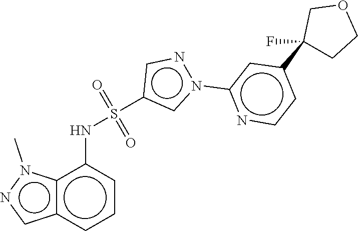 |
| I-336 | 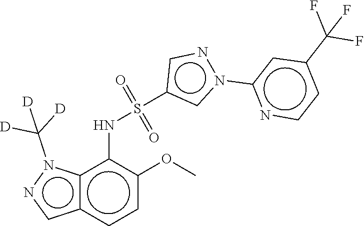 |
| I-337 | 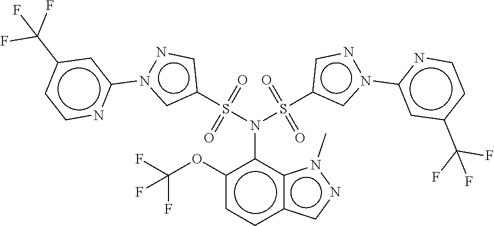 |
| I-338 | 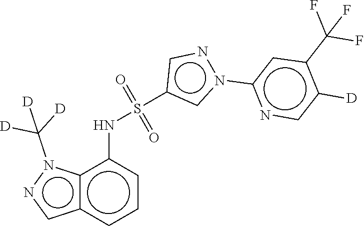 |
| I-339 | 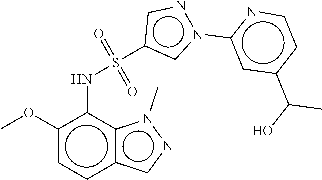 |
| I-340 | 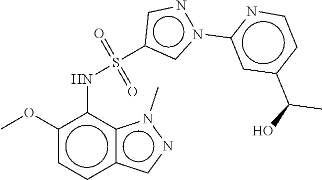 |
| I-341 | 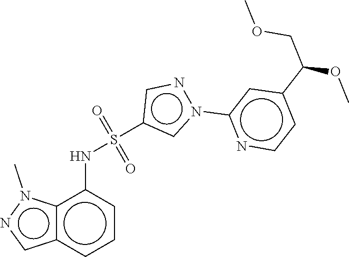 |
| I-342 | 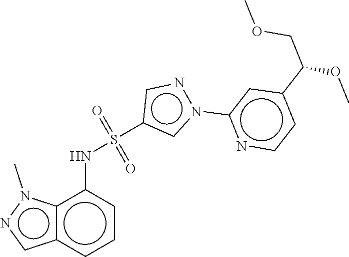 |
| I-343 | 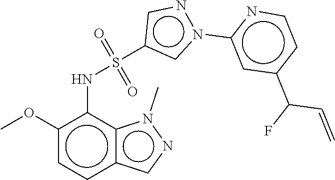 |
| I-344 | 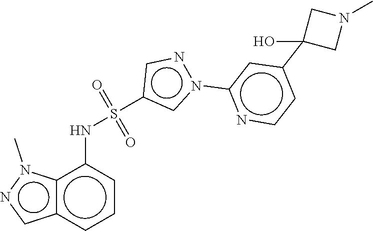 |
| I-345 | 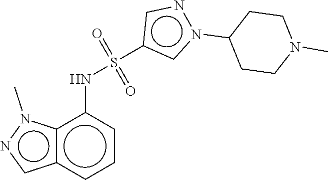 |
| I-346 | 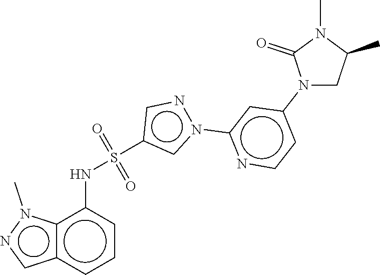 |
| I-347 | 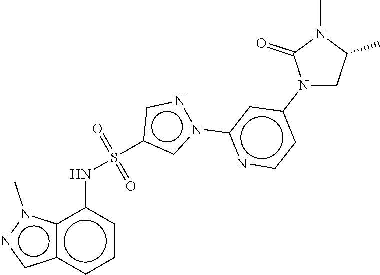 |
| I-348 | 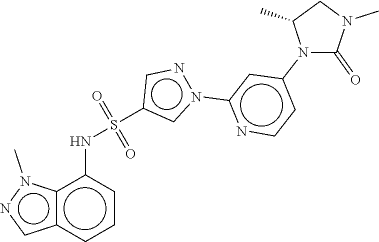 |
| I-349 | 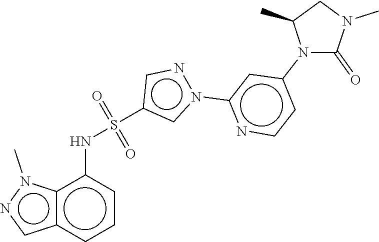 |
| I-350 | 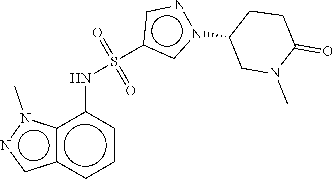 |
| I-351 | 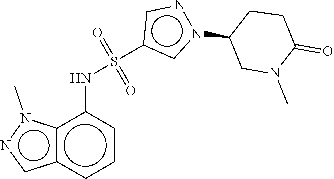 |
| I-352 | 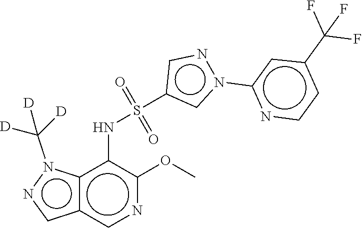 |
| I-353 | 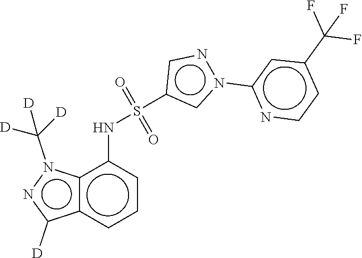 |
| I-354 | 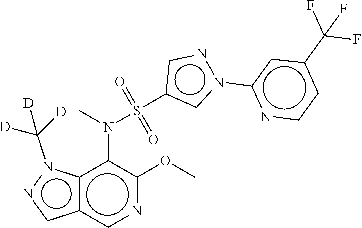 |
| I-355 | 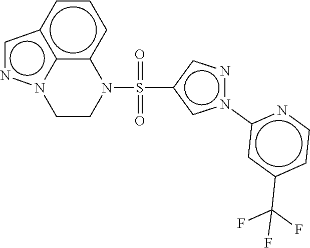 |
| I-356 | 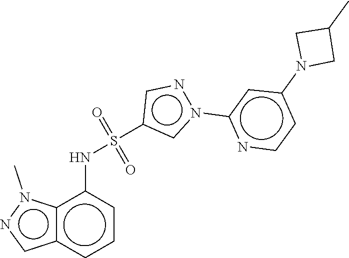 |
| I-357 | 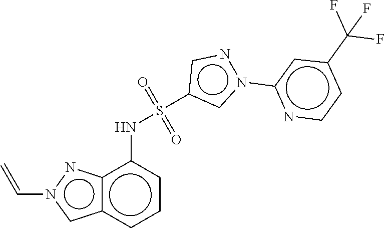 |
| I-358 | 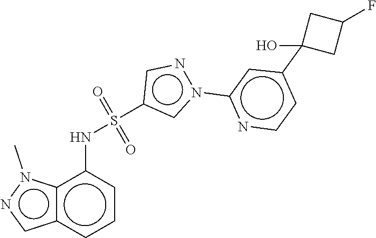 |
| I-359 | 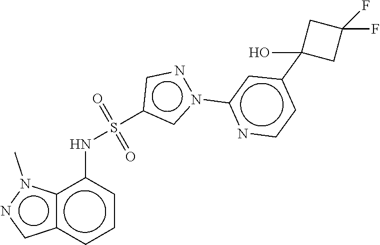 |
| I-360 | 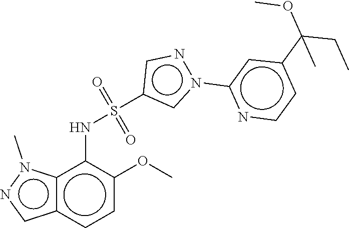 |
| I-361 | 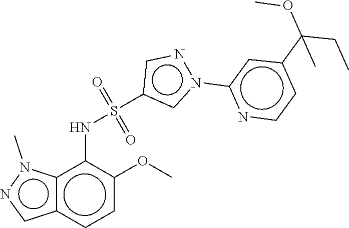 |
| I-362 | 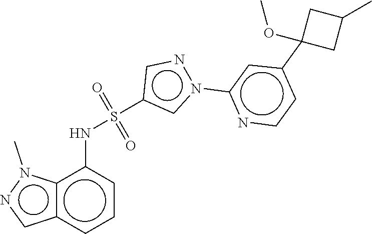 |
| I-363 | 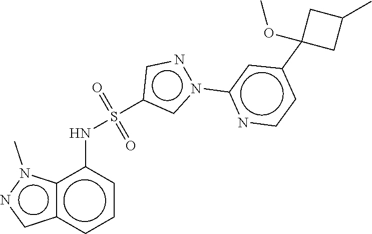 |
| I-364 | 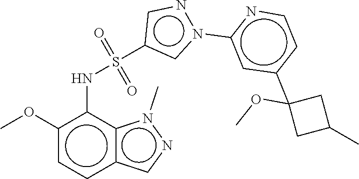 |
| I-365 | 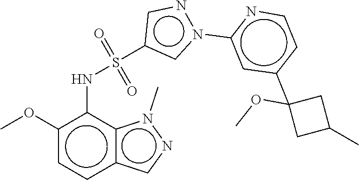 |
| I-366 | 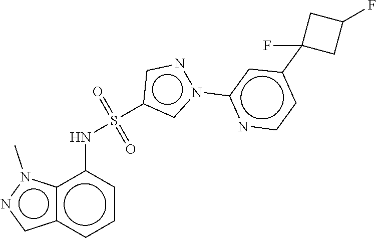 |
| I-367 | 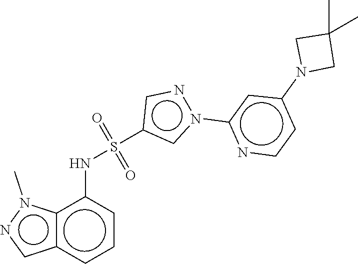 |
| I-368 | 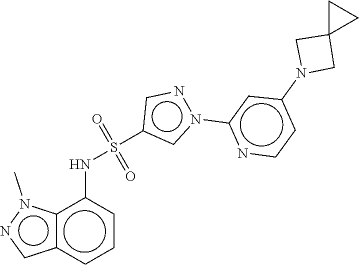 |
| I-369 | 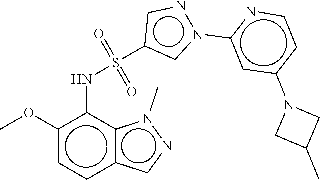 |
| I-370 | 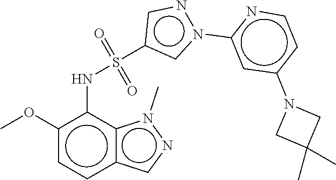 |
| I-371 | 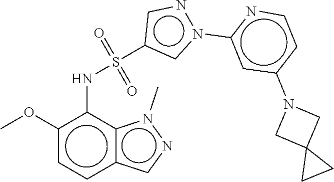 |
| I-372 | 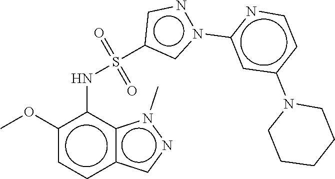 |
| I-373 | 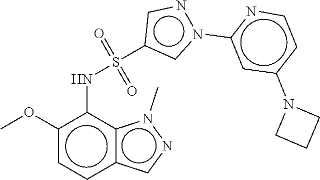 |
| I-374 | 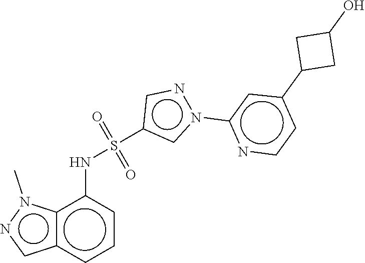 |
| I-375 | 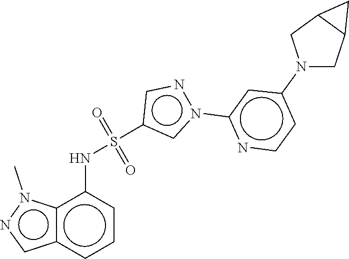 |
| I-376 | 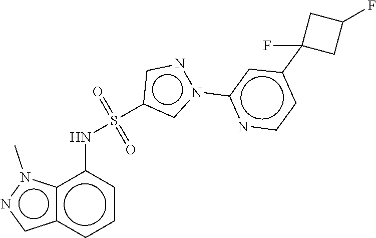 |
| I-377 | 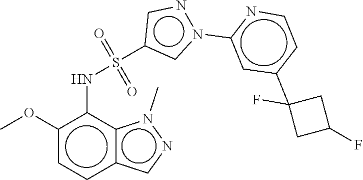 |
| I-378 | 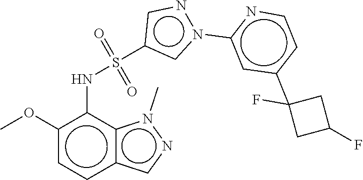 |
| I-379 | 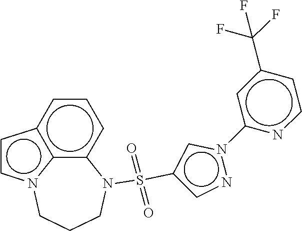 |
| I-380 | 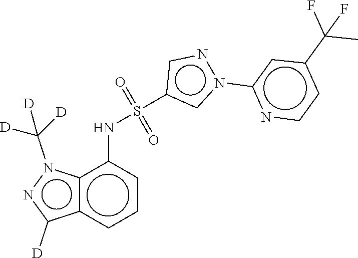 |
| I-381 | 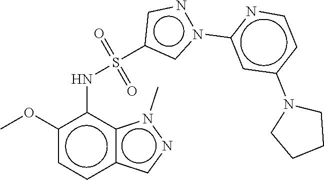 |
| I-382 | 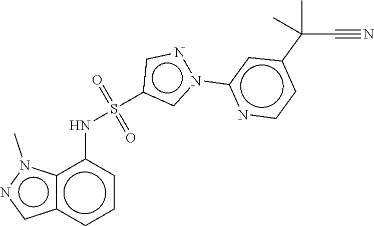 |
| I-383 | 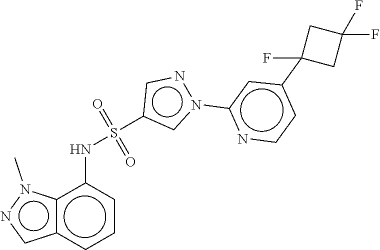 |
| I-384 | 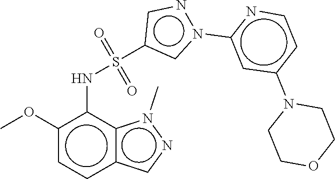 |
| I-385 | 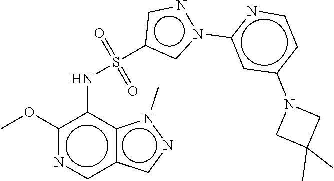 |
| I-386 | 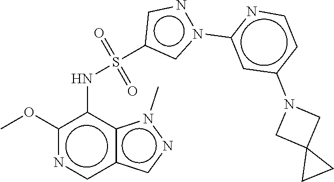 |
| I-387 | 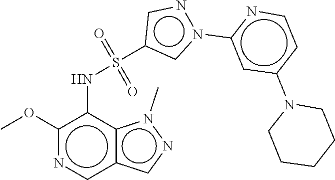 |
| I-388 | 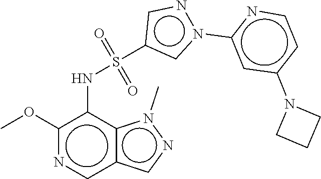 |
| I-389 | 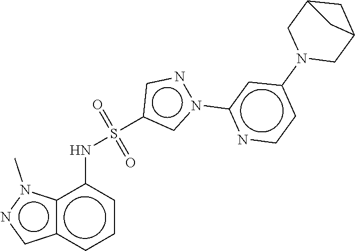 |
| I-390 | 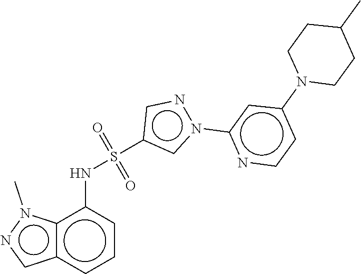 |
| I-391 | 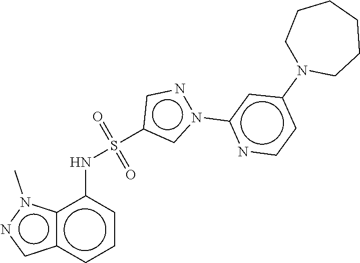 |
| I-392 | 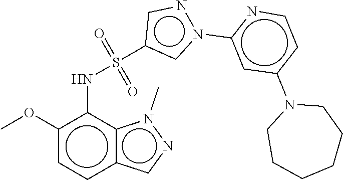 |
| I-393 | 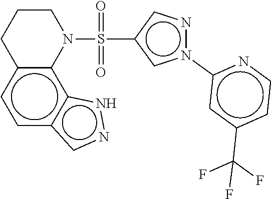 |
| I-394 | 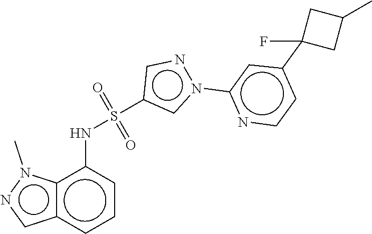 |
| I-395 | 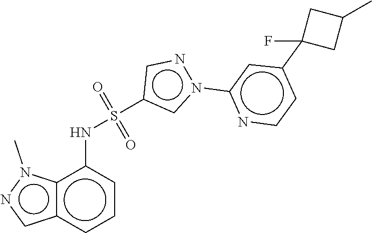 |
| I-396 | 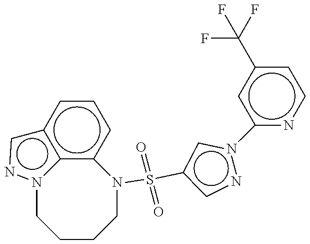 |
| I-397 | 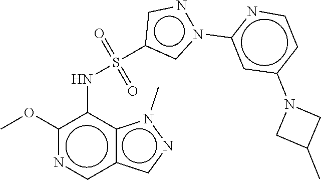 |
| I-398 | 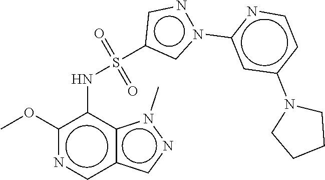 |
| I-399 | 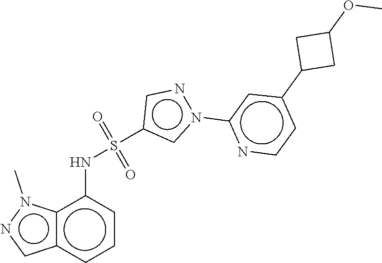 |
| I-400 | 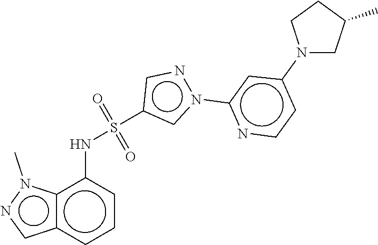 |
| I-401 | 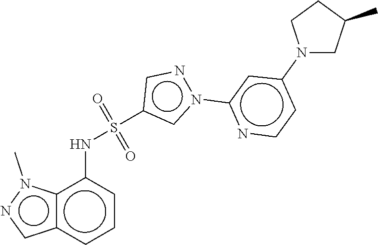 |
| I-402 | 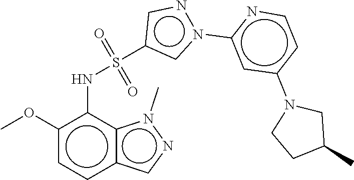 |
| I-403 | 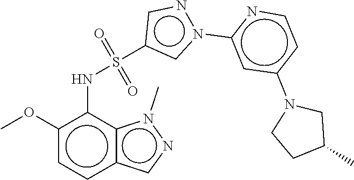 |
| I-404 | 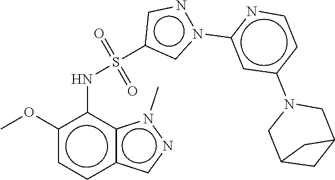 |
| I-405 | 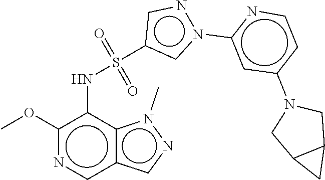 |
| I-406 | 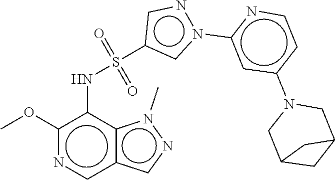 |
| I-407 | 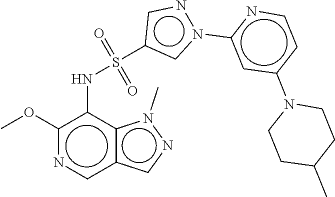 |
| I-408 | 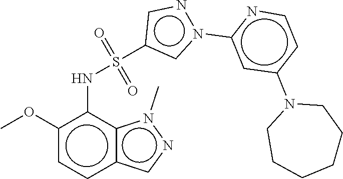 |
| I-409 | 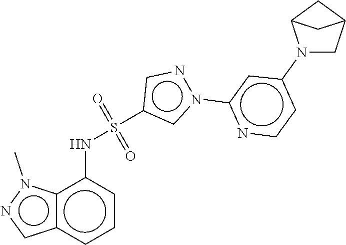 |
| I-410 | 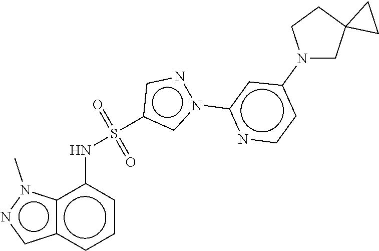 |
| I-411 | 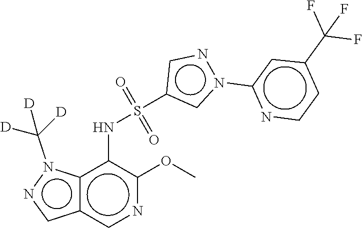 |
| I-412 | 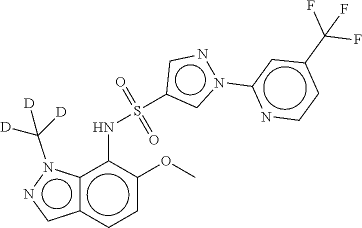 |
| I-413 | 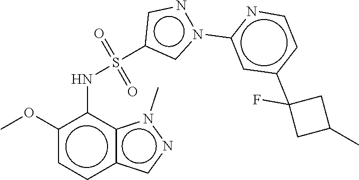 |
| I-414 | 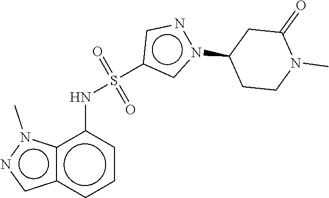 |
| I-415 | 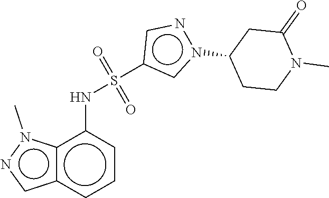 |
| I-416 | 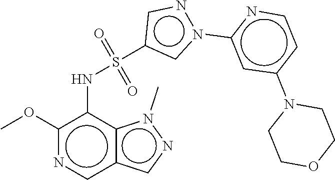 |
| I-417 | 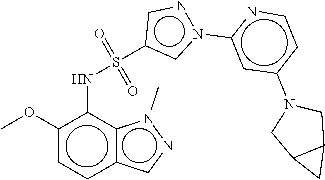 |
| I-418 | 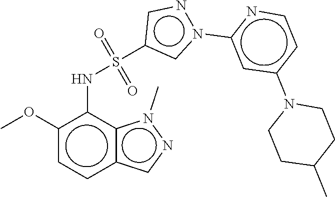 |
| I-419 | 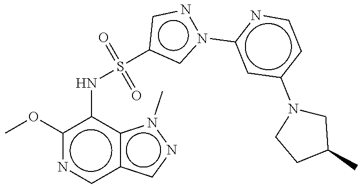 |
| I-420 | 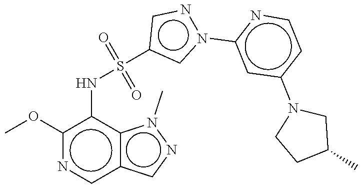 |
| I-421 | 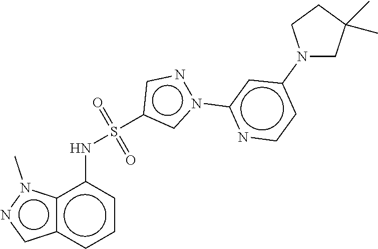 |
| I-422 | 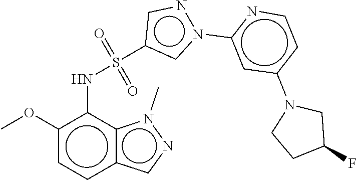 |
| I-423 | 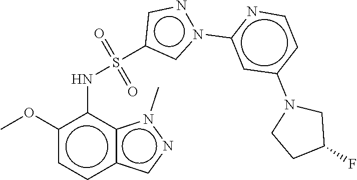 |
| I-424 | 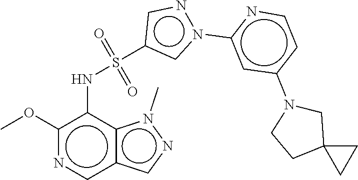 |
| I-425 | 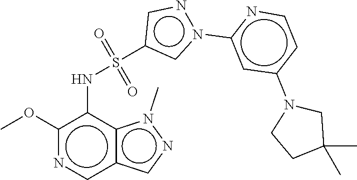 |
| I-426 | 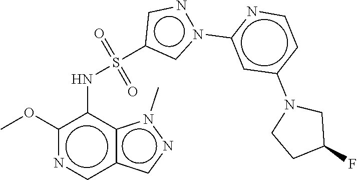 |
| I-427 | 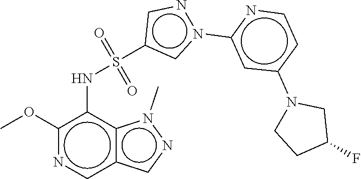 |
| I-428 | 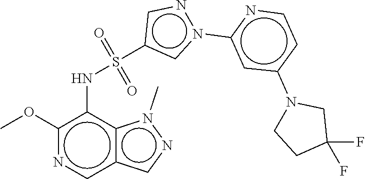 |
| I-429 | 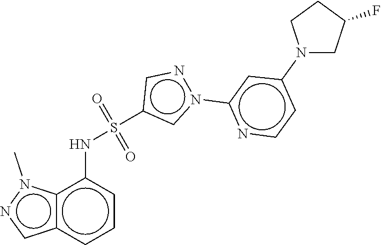 |
| I-430 | 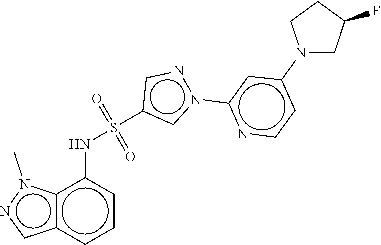 |
| I-431 | 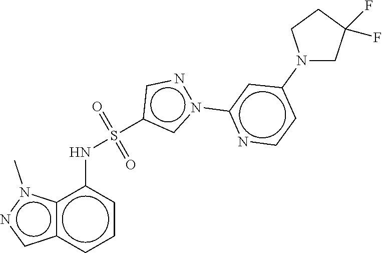 |
| I-432 | 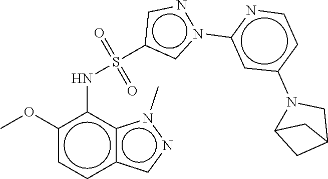 |
| I-433 | 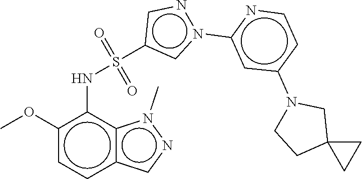 |
| I-434 | 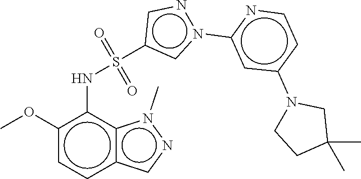 |
| I-435 | 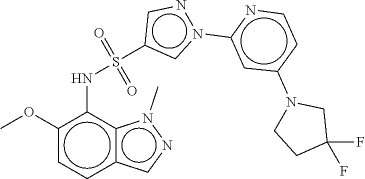 |
| I-436 | 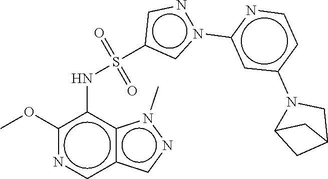 |
| I-437 | 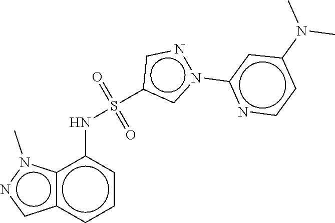 |
| I-438 | 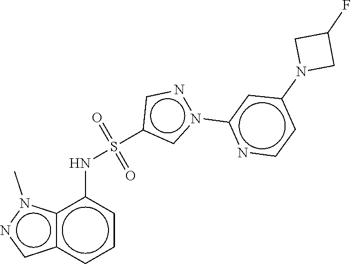 |
| I-439 | 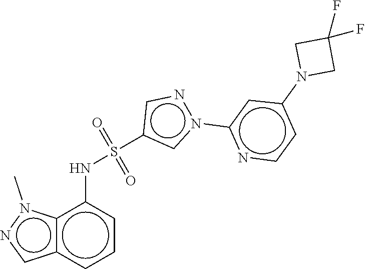 |
| I-440 | 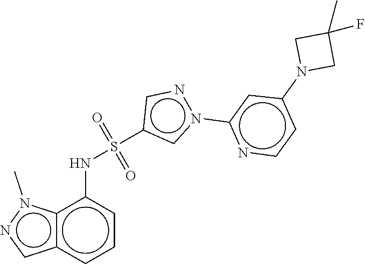 |
| I-441 | 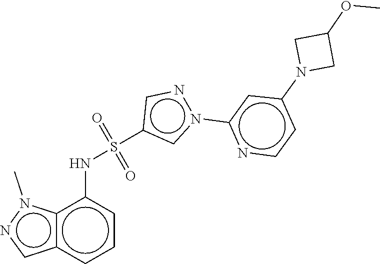 |
| I-442 | 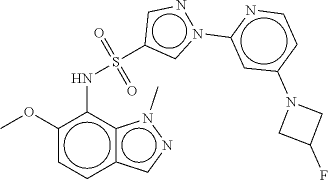 |
| I-443 | 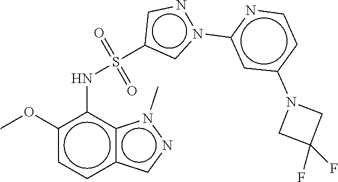 |
| I-444 | 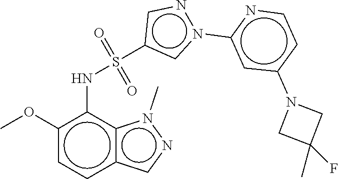 |
| I-445 | 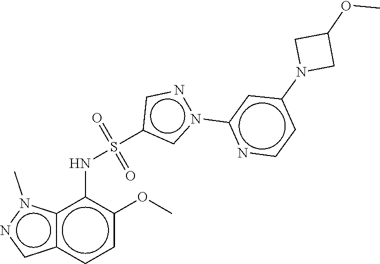 |
| I-446 | 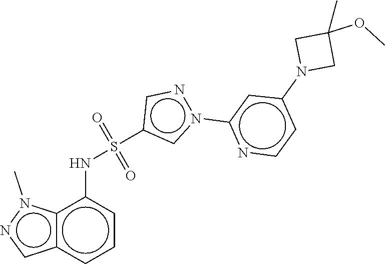 |
| I-447 | 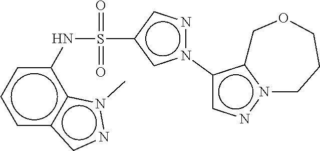 |
| I-448 | 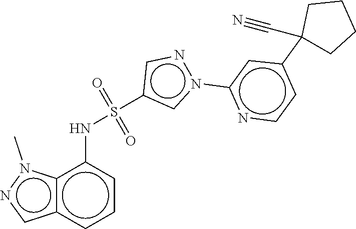 |
| I-449 | 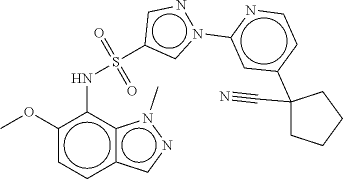 |
| I-450 | 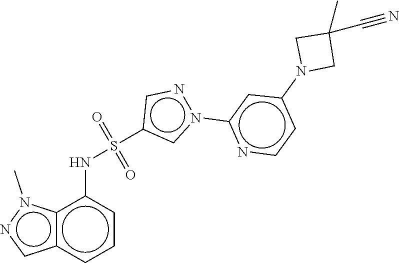 |
| I-451 | 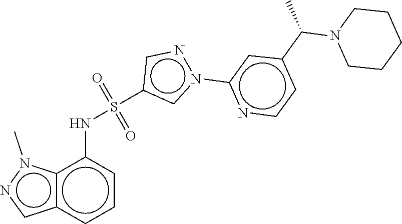 |
| I-452 | 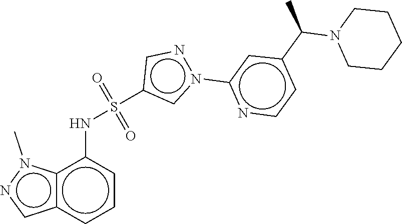 |
| I-453 | 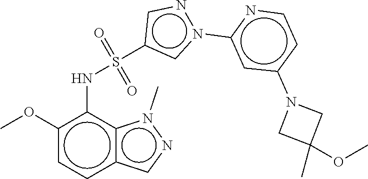 |
| I-454 | 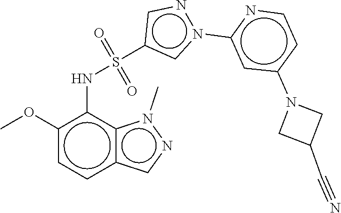 |
| I-455 | 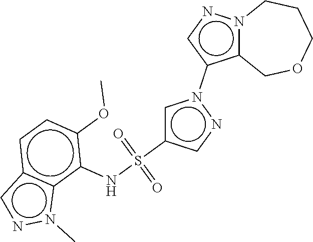 |
| I-456 | 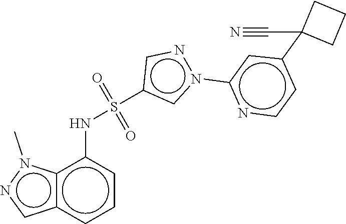 |
| I-457 | 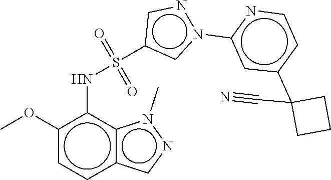 |
| I-458 | 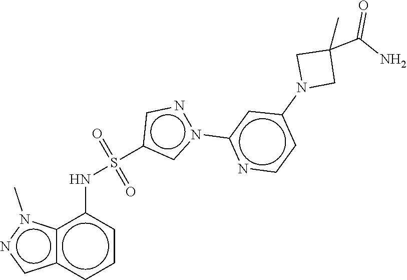 |
| I-459 | 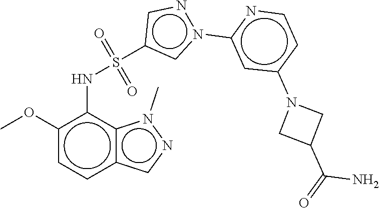 |
| I-460 | 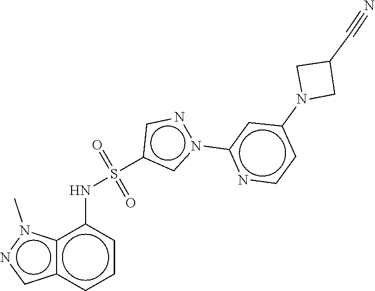 |
| I-461 | 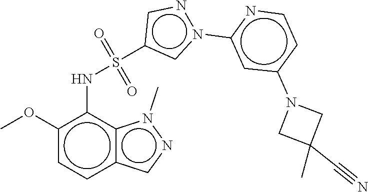 |
| I-462 | 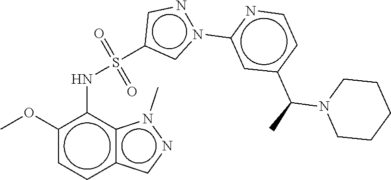 |
| I-463 | 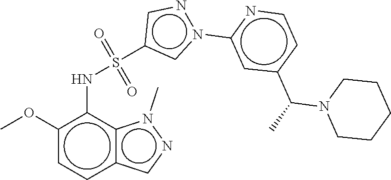 |
| I-464 | 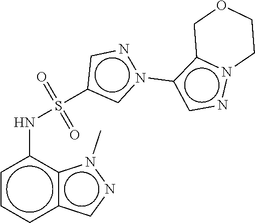 |
| I-465 | 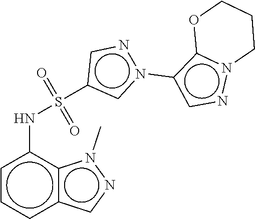 |
| I-466 | 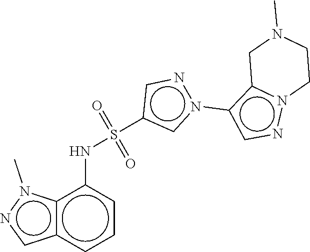 |
| I-467 | 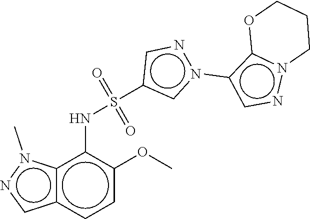 |
| I-468 | 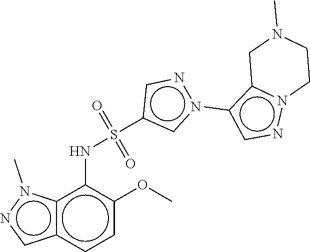 |
| I-469 | 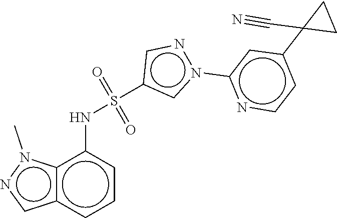 |
| I-470 | 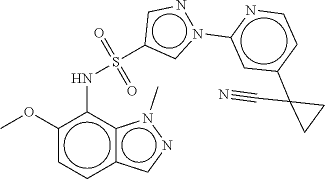 |
| I-471 | 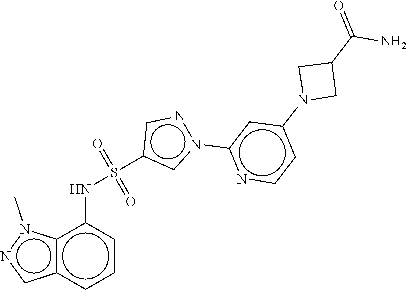 |
| I-472 | 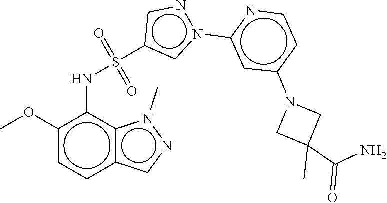 |
| I-473 | 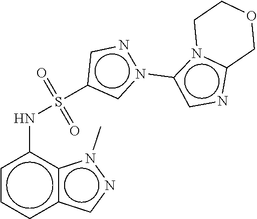 |
| I-474 | 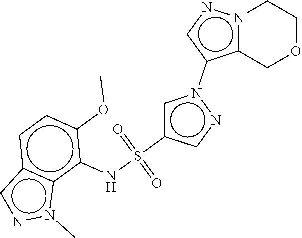 |
| I-475 | 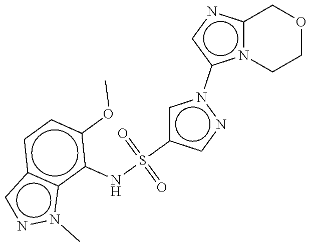 |
| I-476 | 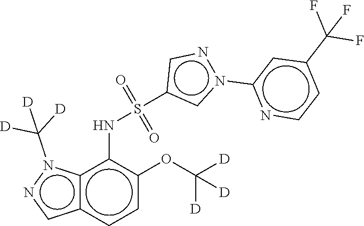 |
| I-477 | 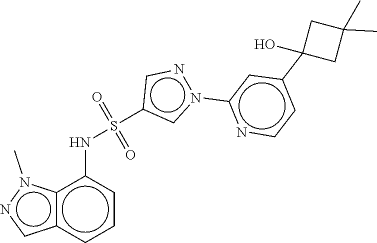 |
| I-478 | 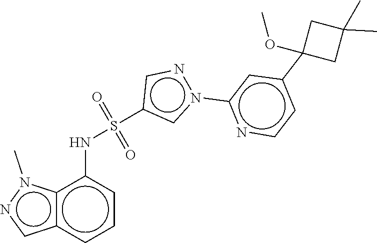 |
| I-479 | 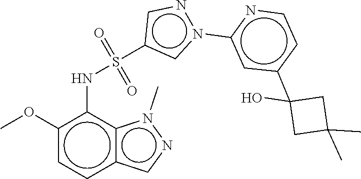 |
| I-480 | 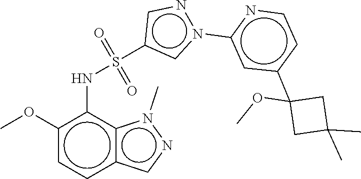 |
| I-481 | 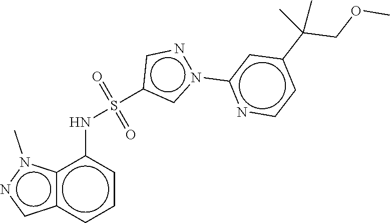 |
| I-482 | 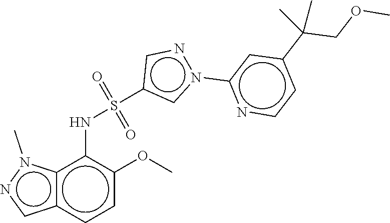 |
| I-483 | 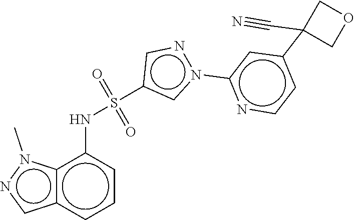 |
| I-484 | 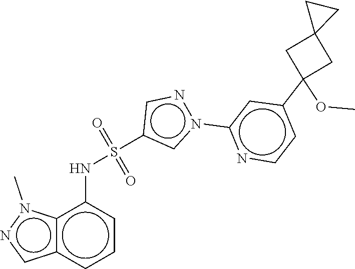 |
| I-485 | 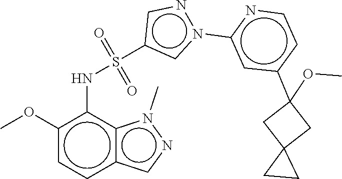 |
| I-486 | 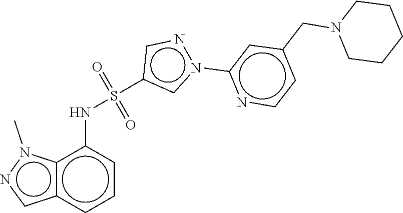 |
| I-487 | 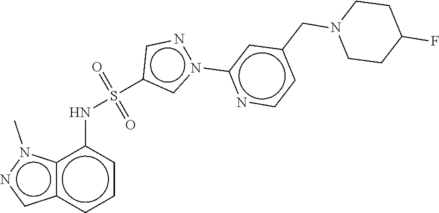 |
| I-488 | 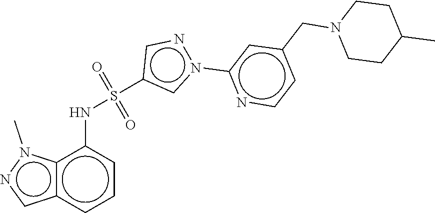 |
| I-489 | 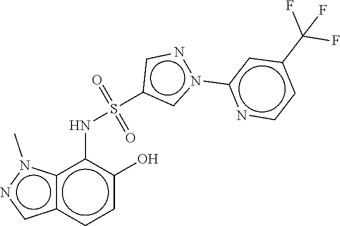 |
| I-490 | 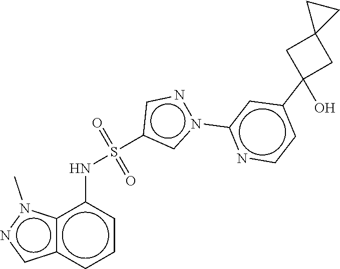 |
| I-491 | 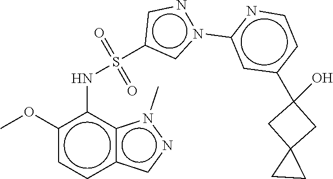 |
| I-492 | 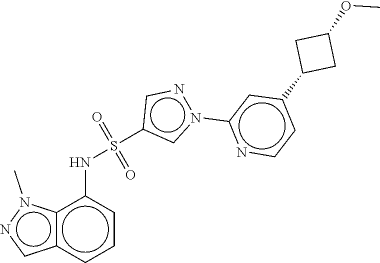 |
| I-493 | 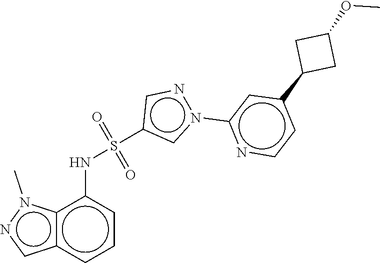 |
| I-494 | 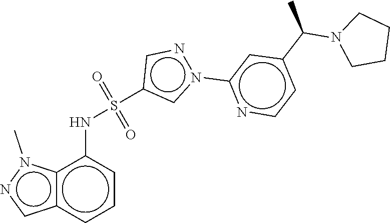 |
| I-495 | 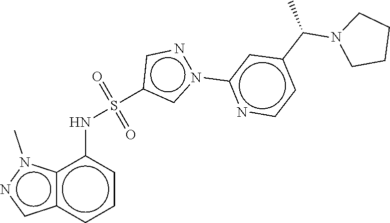 |
| I-496 | 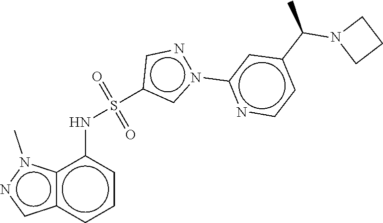 |
| I-497 | 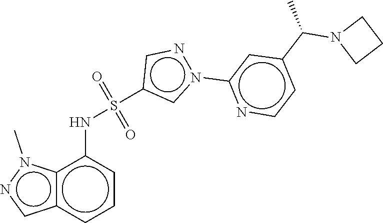 |
| I-498 | 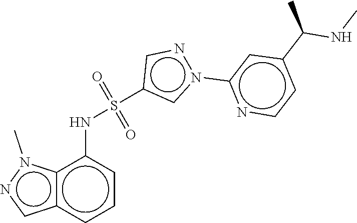 |
| I-499 | 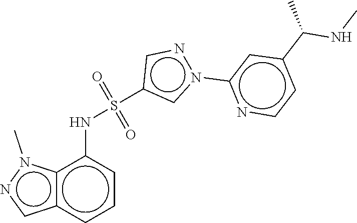 |
| I-500 | 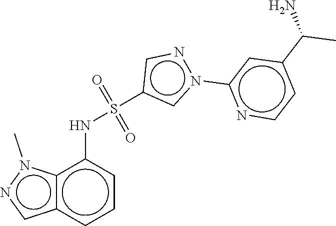 |
| I-501 | 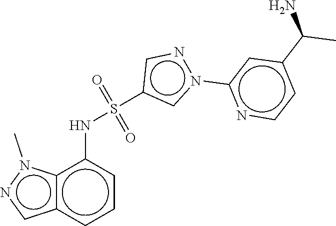 |

In certain embodiments, the compound is

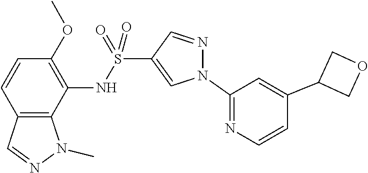 [SMILES: COc1ccc2cnn(C)c2c1NS(=O)(=O)c1cnn(-c2cc(C3COC3)ccn2)c1]

or a pharmaceutically acceptable salt thereof.

### Part D:

Another aspect of the invention provides a compound represented by Formula II:

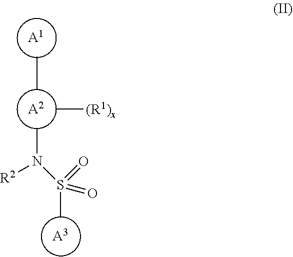

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteratoms independently selected from oxygen, nitrogen, and sulfur, wherein the heteroaryl and heterocyclyl are substituted with n occurrences of R⁶;
A² is pyrazolylene or 1,2,3-triazolylene;
A³ is

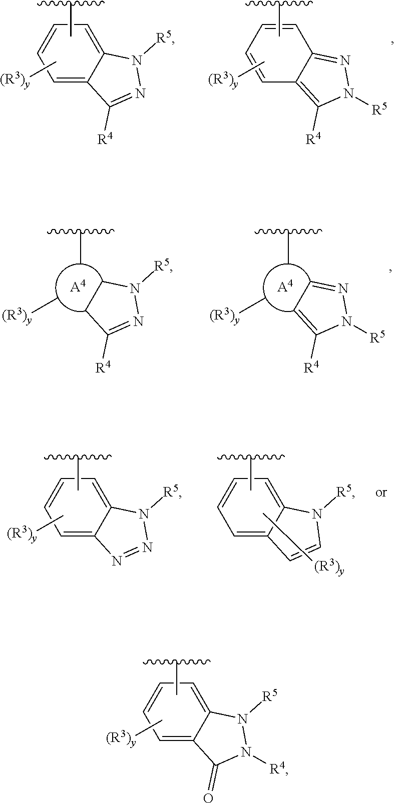

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, or —S(O₂)R¹⁰;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

The definitions of variables in Formula II above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula II.

As defined generally above, A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶. In certain embodiments, A¹ is a 6-membered heteroaryl containing 1, 2, or 3 heteratoms independently selected from oxygen, nitrogen, and sulfur, wherein the heteroaryl is substituted with n occurrences of R⁶. In certain embodiments, A¹ is pyridinyl, pyrimidinyl, pyridazinyl, or pyrazinyl, each of which is substituted with n occurrences of R⁶. In certain embodiments, A¹ is pyridinyl substituted with n occurrences of R⁶. In certain embodiments, A¹ is

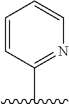 [SMILES: *c1ccccn1]

substituted with n occurrences of R⁶. In certain embodiments, A¹ is

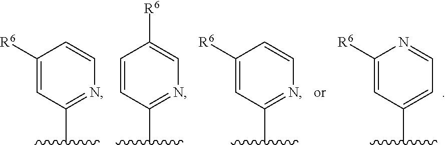

In certain embodiments, A¹ is

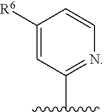

In certain embodiments, A¹ is

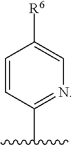

In certain embodiments, A¹ is

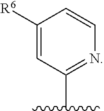

In certain embodiments, A¹ is

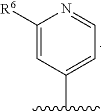

In certain embodiments, A¹ is is

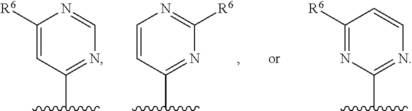

In certain embodiments, A¹ is is

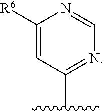

In certain embodiments, A¹ is

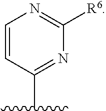

In certain embodiments, A¹ is

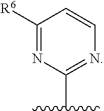

In certain embodiments, A¹ is a 5-6 membered heteroaryl containing 1, 2, or 3 heteratoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl are substituted with n occurrences of R⁶. In certain embodiments, A¹ is a 5-membered heteroaryl containing 1, 2, or 3 heteratoms independently selected from oxygen, nitrogen, and sulfur, wherein the heteroaryl is substituted with n occurrences of R⁶. In certain embodiments, A¹ is a 9-10 membered bicyclic heteroaryl containing 1, 2, or 3 heteroatoms independently selected from oxygen, nitrogen, and sulfur. In certain embodiments, A¹ is a 9-10 membered bicyclic heteroaryl containing 1 heteroatom selected from oxygen, nitrogen, and sulfur. In certain embodiments, A¹ is a 9-10 membered bicyclic heteroaryl containing 2 heteroatoms independently selected from oxygen, nitrogen, and sulfur. In certain embodiments, A¹ is a 9-10 membered bicyclic heteroaryl containing 3 heteroatoms independently selected from oxygen, nitrogen, and sulfur. In certain embodiments, A¹ is a 3-10 membered saturated heterocyclyl containing 1, 2, or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom independently selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶. In certain embodiments, A¹ is selected from the groups depicted in the compounds in Table 2 below.

As defined generally above, A² is pyrazolylene or 1,2,3-triazolylene. In certain embodiments, A² is pyrazolylene. In certain embodiments, A² is 1,2,3-triazolylene.

In certain embodiments, A² is

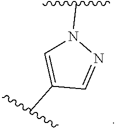 [SMILES: *c1cnn(*)c1]

In certain embodiments, A² is

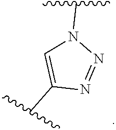 [SMILES: *c1cn(*)nn1]

In certain embodiments, A² is selected from the groups depicted in the compounds in Table 2 below.

As defined generally above, A³ is

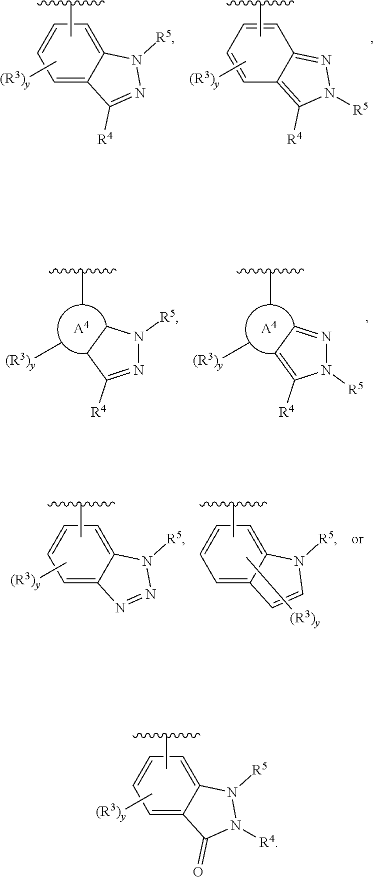

In certain embodiments, A³ is

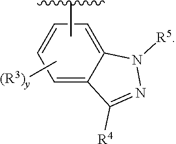

In certain embodiments, A³ is

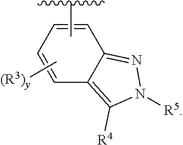

In certain embodiments, A³

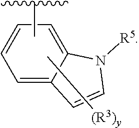

In certain embodiments, A³ is

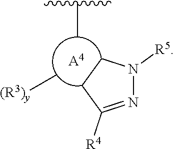

In certain embodiments, A³ is

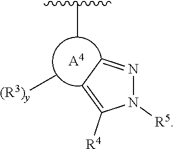

In certain embodiments, A³ is

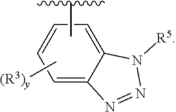

In certain embodiments, A³ is

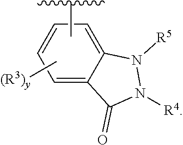

In certain embodiments, A³ is selected from the groups depicted in the compounds in Table 2 below.

As defined generally above, A⁴ is a 6-membered aromatic ring containing 1 nitrogen atom. In certain embodiments, A⁴ is selected from the groups depicted in the compounds in Table 2 below.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 2 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R⁵ is C₁. 4 alkyl. In certain embodiments, R⁵ is methyl. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 2 below.

As defined generally above, R⁵ is hydrogen, C₁₋₄ alkyl, or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 2 below.

In certain embodiments, R⁵ is selected from the groups depicted in the compounds in Table 2 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, or —S(O₂)R¹⁰. In certain embodiments, R³ is C₁₋₆ alkyl. In certain embodiments, R³ is ethyl. In certain embodiments, C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl In certain embodiments, R³ is C₁₋₆ haloalkyl. In certain embodiments, R³ is C₁₋₆ deuteroalkyl. In certain embodiments, R³ is C₁₋₆ deuteroalkoxyl. In certain embodiments, R³ is —O—C₃₋₇ cycloalkyl. In certain embodiments, R³ is —(C₀₋₄ alkylene)-CN. In certain embodiments, R³ is cyano. In certain embodiments, R³ is C₂₋₄ alkynyl. In certain embodiments, R³ is —N(R⁸)(R⁹). In certain embodiments, R³ is CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, R³ is selected from the groups depicted in the compounds in Table 2 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is F. In certain embodiments, R⁴ is Cl. In certain embodiments, R⁴ is Br. In certain embodiments, R⁴ is I. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 2 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R⁶ is C₁₋₆ haloalkyl. In certain embodiments, R⁶ is —CF₃. In certain embodiments, R⁶ is C₁₋₆ alkyl. In certain embodiments, R⁶ is methyl. In certain embodiments, R⁶ is C₃₋₇ cycloalkyl. In certain embodiments, R⁶ is cyclopropyl. In certain embodiments, R⁶ is halo. In certain embodiments, R⁶ is hydroxyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is C₃₋₇ halocycloalkyl. In certain embodiments, R⁶ is C₃₋₇ hydroxycycloalkyl, In certain embodiments, R⁶ is —N(R⁸)(R⁹). In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R³ is a 3-7 membered saturated heterocyclyl containing 1 or 2 heteroatoms independently selected from oxygen and nitrogen, wherein the heterocyclyl is substituted with 0 or 1 halo. In certain embodiments, R³ is a 3-7 membered saturated heterocyclyl containing 1 heteroatom selected from oxygen and nitrogen, wherein the heterocyclyl is substituted with 0 or 1 halo. In certain embodiments, R³ is a 3-7 membered saturated heterocyclyl containing 2 heteroatoms independently selected from oxygen and nitrogen, wherein the heterocyclyl is substituted with 0 or 1 halo. In certain embodiments, R⁶ is selected from the groups depicted in the compounds in Table 2 below.

As defined generally above, R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteratoms independently selected from oxygen, nitrogen, and sulfur, wherein the heterocyclyl is substituted with m occurrences of R¹¹. In certain embodiments, R⁷ is a 4-6 membered saturated heterocyclyl containing 1 heteratom selected from oxygen, nitrogen, and sulfur, wherein the heterocyclyl is substituted with m occurrences of R¹¹. In certain embodiments, R⁷ is a 4-6 membered saturated heterocyclyl containing 2 heteratoms independently selected from oxygen, nitrogen, and sulfur, wherein the heterocyclyl is substituted with m occurrences of R¹¹. In certain embodiments, R⁷ is selected from the groups depicted in the compounds in Table 2 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 2 below. In certain embodiments, R⁹ is selected from the groups depicted in the compounds in Table 2 below.

As defined generally above, R¹⁰ represents independently for each occurrence C₁₋₆ alkyl or (C₀₋₅ alkylene)-C₃₋₇ cycloalkyl. In certain embodiments, R¹⁰ is C₁₋₆ alkyl. In certain embodiments, R¹⁰ is (C₀₋₅ alkylene)-C₃₋₇ cycloalkyl. In certain embodiments, R¹⁰ is selected from the groups depicted in the compounds in Table 2 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is F. In certain embodiments, R¹¹ is Cl. In certain embodiments, R¹¹ is Br. In certain embodiments, R¹¹ is I. In certain embodiments, R¹¹ is hydroxyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 2 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 2 below.

As defined generally above, n, m, and x are independently 0, 1, or 2. In certain embodiments, n and x are independently 0, 1, or 2. In certain embodiments, n and x are independently 0, 1, or 2. In certain embodiments, x is 0. In certain embodiments, n is 1. In certain embodiments, n is 2. In certain embodiments, n is 0. In certain embodiments, x is 1. In certain embodiments, x is 2. In certain embodiments, m is 0. In certain embodiments, m is 1. In certain embodiments, m is 2. In certain embodiments, n is selected from the corresponding value in the groups depicted in the compounds in Table 2 below. In certain embodiments, m is selected from the corresponding value in the groups depicted in the compounds in Table 1 below. In certain embodiments, x is selected from the corresponding value in the groups depicted in the compounds in Table 2 below.

The description above describes multiple embodiments relating to compounds of Formula II. The patent application specifically contemplates all combinations of the embodiments.

### Part E:

Another aspect of the invention provides a compound in Table 2 below, or a pharmaceutically acceptable salt thereof. In certain embodiments, the compound is a compound in Table 2. In certain embodiments, the compound is any one of compounds II-1 to II-20 in Table 2 or a pharmaceutically acceptable salt thereof. In certain embodiments, the compound is any one of compounds II-1 to II-20 in Table 2. In certain embodiments, the compound is compound II-21 in Table 2 or a pharmaceutically acceptable salt thereof. In certain embodiments, the compound is compound II-21 in Table 2.

TABLE 2

| Compound | |
|---|---|
| No. | Structure |
| II-1 | 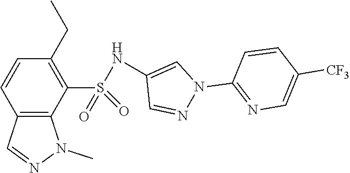 [SMILES: CCc1ccc2cnn(C)c2c1S(=O)(=O)Nc1cnn(-c2ccc(C(F)(F)F)cn2)c1] |
| II-2 | 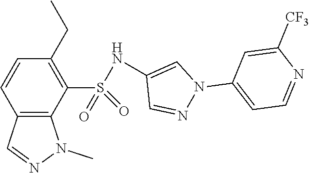 [SMILES: CCc1ccc2cnn(C)c2c1S(=O)(=O)Nc1cnn(-c2ccnc(C(F)(F)F)c2)c1] |
| II-3 | 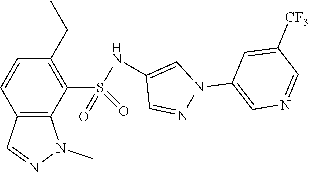 [SMILES: CCc1ccc2cnn(C)c2c1S(=O)(=O)Nc1cnn(-c2cncc(C(F)(F)F)c2)c1] |
| II-4 | 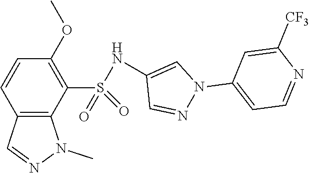 [SMILES: COc1ccc2cnn(C)c2c1S(=O)(=O)Nc1cnn(-c2ccnc(C(F)(F)F)c2)c1] |
| II-5 | 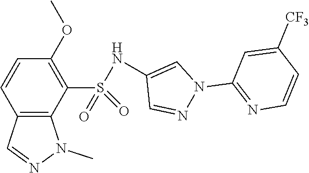 [SMILES: COc1ccc2cnn(C)c2c1S(=O)(=O)Nc1cnn(-c2cc(C(F)(F)F)ccn2)c1] |
| II-6 | 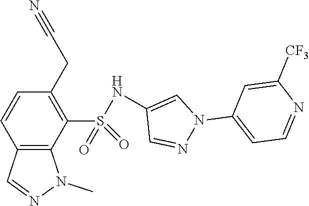 [SMILES: Cn1ncc2ccc(CC#N)c(S(=O)(=O)Nc3cnn(-c4ccnc(C(F)(F)F)c4)c3)c21] |
| II-7 | 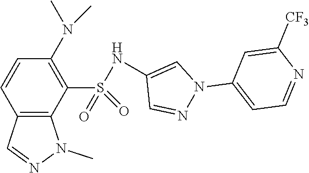 [SMILES: CN(C)c1ccc2cnn(C)c2c1S(=O)(=O)Nc1cnn(-c2ccnc(C(F)(F)F)c2)c1] |
| II-8 | 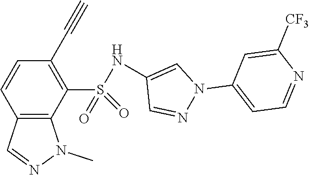 [SMILES: C#Cc1ccc2cnn(C)c2c1S(=O)(=O)Nc1cnn(-c2ccnc(C(F)(F)F)c2)c1] |
| II-9 | 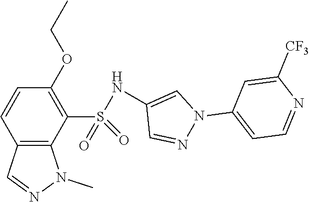 [SMILES: CCOc1ccc2cnn(C)c2c1S(=O)(=O)Nc1cnn(-c2ccnc(C(F)(F)F)c2)c1] |
| II-10 | 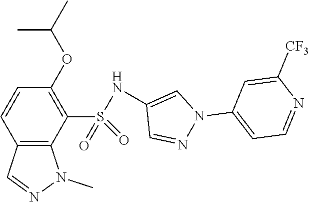 [SMILES: CC(C)Oc1ccc2cnn(C)c2c1S(=O)(=O)Nc1cnn(-c2ccnc(C(F)(F)F)c2)c1] |
| II-11 | 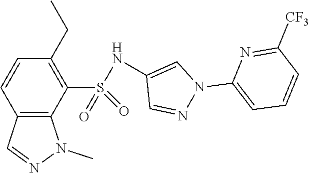 [SMILES: CCc1ccc2cnn(C)c2c1S(=O)(=O)Nc1cnn(-c2cccc(C(F)(F)F)n2)c1] |
| II-12 | 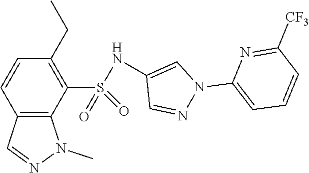 [SMILES: CCc1ccc2cnn(C)c2c1S(=O)(=O)Nc1cnn(-c2cccc(C(F)(F)F)n2)c1] |
| II-13 | 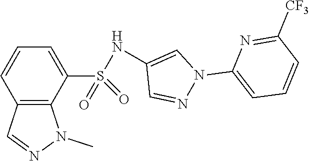 [SMILES: Cn1ncc2cccc(S(=O)(=O)Nc3cnn(-c4cccc(C(F)(F)F)n4)c3)c21] |
| II-14 | 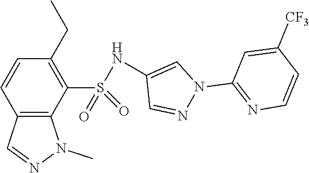 [SMILES: CCc1ccc2cnn(C)c2c1S(=O)(=O)Nc1cnn(-c2cc(C(F)(F)F)ccn2)c1] |
| II-15 | 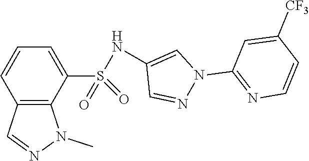 [SMILES: Cn1ncc2cccc(S(=O)(=O)Nc3cnn(-c4cc(C(F)(F)F)ccn4)c3)c21] |
| II-16 | 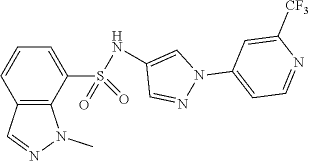 [SMILES: Cn1ncc2cccc(S(=O)(=O)Nc3cnn(-c4ccnc(C(F)(F)F)c4)c3)c21] |
| II-17 | 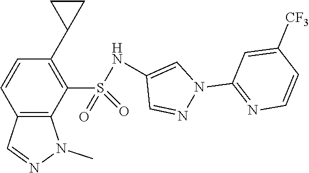 [SMILES: Cn1ncc2ccc(C3CC3)c(S(=O)(=O)Nc3cnn(-c4cc(C(F)(F)F)ccn4)c3)c21] |
| II-18 | 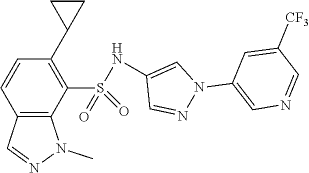 [SMILES: Cn1ncc2ccc(C3CC3)c(S(=O)(=O)Nc3cnn(-c4cncc(C(F)(F)F)c4)c3)c21] |
| II-19 | 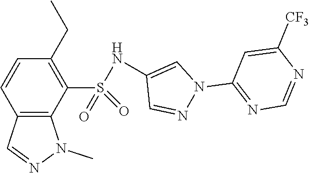 [SMILES: CCc1ccc2cnn(C)c2c1S(=O)(=O)Nc1cnn(-c2cc(C(F)(F)F)ncn2)c1] |
| II-20 | 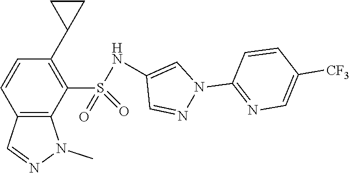 [SMILES: Cn1ncc2ccc(C3CC3)c(S(=O)(=O)Nc3cnn(-c4ccc(C(F)(F)F)cn4)c3)c21] |
| II-21 | 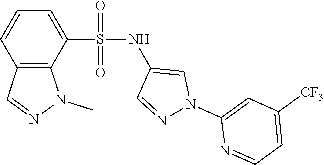 [SMILES: Cn1ncc2cccc(S(=O)(=O)Nc3cnn(-c4cc(C(F)(F)F)ccn4)c3)c21] |

Methods for preparing compounds described herein are illustrated in the following synthetic scheme. The scheme is provided for the purpose of illustrating the invention, and is not intended to limit the scope or spirit of the invention. Starting materials shown in the scheme can be obtained from commercial sources or can be prepared based on procedures described in the literature.

In the scheme, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in Protecting Groups in Organic Synthesis, 3^{rd} Edition, T. W. Greene and P. G. M. Wuts, John Wiley & Sons, 1999 and Greene's Protective Groups in Organic Synthesis, 5^{th} Ed., (Peter G. M. Wuts, John Wiley & Sons: 2014), the entire contents of both of which are hereby incorporated by reference.

The synthetic route illustrated in Scheme 1 is a general method for preparing pyrazolyl sulfonamides C. Reaction of pyrazolyl sulfonylchloride A with amine B provides pyrazolyl sulfonamide C.

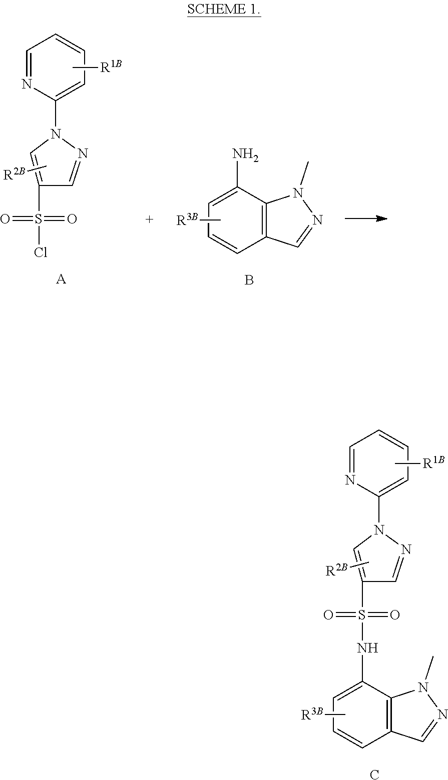

The modular synthetic route illustrated in Scheme 1 can be adjusted to provide additional pyridinylsulfonamide compounds by conducting functional group transformations on the intermediate and final compounds. Such functional group transformations are well known in the art, as described in, for example, Comprehensive Organic Synthesis (B. M. Trost & I. Fleming, eds., 1991-1992); Organic Synthesis, 3^{rd} Ed. (Michael B. Smith, Wavefunction, Inc., Irvine: 2010); Modern Methods of Organic Synthesis, 4^{th} Ed. (William Carruthers and lain Coldham, Cambridge University Press, Cambridge: 2004); March 's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3^{rd} Ed. (Richard C. Larock, ed., John Wiley & Sons, New York: 2018).

### II. Therapeutic Applications of Pyrazolylsulfonamide Compounds

Compounds described herein are useful for treating a disease or condition mediated by MALT1. Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

Accordingly, one aspect of the invention provides a method of treating a disease or condition mediated by MALT1 in a subject. The method comprises administering to a subject in need thereof a therapeutically effective amount of a compound described herein, such as a compound of Formula I, I-1, I-2, I-3, I-4, or II, or other compounds in Section I, to treat the disease or condition. In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above. Further description of exemplary diseases or conditions mediated by MALT1 is provided herein below.

Another aspect of the invention provides a method of inhibiting the activity of MALT1. The method comprises contacting a MALT1 with an effective amount of a compound described herein, such as a compound of Formula I, I-1, I-2, I-3, I-4, or II, or other compounds in Section I, to inhibit the activity of said MALT1. In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above.

Another aspect of the invention provides for the use of a compound described herein (such as a compound of Formula I, I-1, I-2, I-3, I-4, or II, or other compounds in Section I) in the manufacture of a medicament. In certain embodiments, the medicament is for treating a disease or condition described herein, such as an inflammatory disorder or an allergic disorder. In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above.

Another aspect of the invention provides for the use of a compound described herein (such as a compound of Formula I, I-1, I-2, I-3, I-4, or II, or other compounds in Section I) for treating a disease or condition, such as a disease or condition described herein. In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above.

In certain embodiments, the subject is a human. In certain embodiments, the subject is an adult human. In certain embodiments, the subject is a pediatric human. In certain embodiments, the subject is a geriatric human.

### Exemplary Diseases or Conditions

Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

In certain embodiments, the disease or condition mediated by MALT1 is a proliferative disorder. In certain embodiments, the disease or condition mediated by MALT1 is inflammatory disorder. In certain embodiments, the disease or condition mediated by MALT1 is an autoimmune disorder. In certain embodiments, the disease or condition mediated by MALT1 is a fibrotic disorder. In certain embodiments, the disease or condition mediated by MALT1 is a metabolic disorder. In certain embodiments, the disease or condition mediated by MALT1 is a cardiovascular disorder. In certain embodiments, the disease or condition mediated by MALT1 is a cerebrovascular disorder. In certain embodiments, the disease or condition mediated by MALT1 is a myeloid cell-driven hyper-inflammatory response in a COVID-19 infection.

In certain embodiments, the disease or condition mediated by MALT1 is cancer.

In certain embodiments, the cancer is selected from is non-small cell lung cancer (NSCLC), small cell lung cancer, colorectal cancer, rectal cancer, and pancreatic cancer. In certain embodiments, the cancer is selected from non-small cell lung cancer (NSCLC), pancreatic cancer, and colorectal cancer. In certain embodiments, the cancer is selected from non-small cell lung cancer (NSCLC) and pancreatic cancer.

In certain embodiments, the cancer is a solid tumor. In certain embodiments, the cancer is a melanoma, carcinoma, or blastoma. In certain embodiments, the cancer is a melanoma. In certain embodiments, the cancer is a carcinoma. In certain embodiments, the cancer is an adenocarcinoma. In certain embodiments, the cancer is a blastoma.

In certain embodiments, the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia. In certain embodiments, the cancer is lung cancer. In certain embodiments, the cancer is pancreatic cancer. In certain embodiments, the cancer is colorectal cancer. In certain embodiments, the cancer is breast cancer. In certain embodiments, the cancer is cervical cancer. In certain embodiments, the cancer is prostate cancer. In certain embodiments, the cancer is gastric cancer. In certain embodiments, the cancer is skin cancer. In certain embodiments, the cancer is liver cancer. In certain embodiments, the cancer is bile duct cancer. In certain embodiments, the cancer is nervous system cancer.

In certain embodiments, the cancer is a lymphoma or leukemia. In certain embodiments, the cancer is a B-cell lymphoma or chronic myelocytic leukemia.

In certain embodiments, the cancer is breast adenocarcinoma, lung adenocarcinoma, pancreatic adenocarcinoma, cervical adenocarcinoma, colorectal adenocarcinoma, prostate adenocarcinoma, gastric adenocarcinoma, melanoma, lung squamous cell carcinoma, hepatocellular carcinoma, cholangiocarcinoma, glioblastoma, or neuroblastoma. In certain embodiments, the cancer is breast adenocarcinoma. In certain embodiments, the cancer is lung adenocarcinoma. In certain embodiments, the cancer is pancreatic adenocarcinoma. In certain embodiments, the cancer is cervical adenocarcinoma. In certain embodiments, the cancer is prostate adenocarcinoma. In certain embodiments, the cancer is gastric adenocarcinoma.

In certain embodiments, the cancer is melanoma.

In certain embodiments, the cancer is lung squamous cell carcinoma, hepatocellular carcinoma, or cholangiocarcinoma. In certain embodiments, the cancer is lung squamous cell carcinoma. In certain embodiments, the cancer is hepatocellular carcinoma. In certain embodiments, the cancer is cholangiocarcinoma.

In certain embodiments, the cancer is glioblastoma or neuroblastoma. In certain embodiments, the cancer is glioblastoma. In certain embodiments, the cancer is neuroblastoma.

In certain embodiments, the cancer is lung cancer, pancreatic cancer, or colorectal cancer. In certain embodiments, the cancer is non-small cell lung cancer, pancreatic cancer, or colorectal cancer. In certain embodiments, the cancer is lung cancer. In certain embodiments, the cancer is non-small cell lung cancer.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, or a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In certain embodiments, the cancer is MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

In certain embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In certain embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In certain embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

In certain embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, fallopian tube cancer, papillary serous cystadenocarcinoma, uterine papillary serous carcinoma (UPSC), prostate cancer, testicular cancer, gallbladder cancer, hepatocholangiocarcinoma, soft tissue and bone synovial sarcoma, rhabdomyosarcoma, osteosarcoma, chondrosarcoma, Ewing sarcoma, anaplastic thyroid cancer, adrenocortical adenoma, pancreatic cancer, pancreatic ductal carcinoma, pancreatic adenocarcinoma, gastrointestinal/stomach (GIST) cancer, lymphoma, squamous cell carcinoma of the head and neck (SCCHN), salivary gland cancer, glioma, or brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; and medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In certain embodiments, the cancer is hepatoblastoma. In certain embodiments, the cancer is colon cancer. In certain embodiments, the cancer is rectal cancer. In certain embodiments, the cancer is ovarian cancer, or ovarian carcinoma. In certain embodiments, the cancer is ovarian epithelial cancer. In certain embodiments, the cancer is fallopian tube cancer. In certain embodiments, the cancer is papillary serous cystadenocarcinoma. In certain embodiments, the cancer is uterine papillary serous carcinoma (UPSC). In certain embodiments, the cancer is hepatocholangiocarcinoma. In certain embodiments, the cancer is soft tissue and bone synovial sarcoma. In certain embodiments, the cancer is rhabdomyosarcoma. In certain embodiments, the cancer is osteosarcoma. In certain embodiments, the cancer is anaplastic thyroid cancer. In certain embodiments, the cancer is adrenocortical carcinoma. In certain embodiments, the cancer is pancreatic cancer, or pancreatic ductal carcinoma. In certain embodiments, the cancer is pancreatic adenocarcinoma. In certain embodiments, the cancer is glioma. In certain embodiments, the cancer is malignant peripheral nerve sheath tumors (MPNST). In certain embodiments, the cancer is neurofibromatosis-1 associated MPNST. In certain embodiments, the cancer is Waldenstrom's macroglobulinemia. In certain embodiments, the cancer is medulloblastoma.

In certain embodiments, the cancer is a lymphoma. In certain embodiments, the cancer is a leukemia. In certain embodiments, the cancer is Hodgkin's lymphoma. In certain embodiments, the cancer is non-Hodgkin's lymphoma. In certain embodiments, the cancer is Burkitt's lymphoma. In certain embodiments, the cancer is diffuse large B-cell lymphoma (DLBCL). In certain embodiments, the cancer is MALT lymphoma. In certain embodiments, the cancer is germinal center B-cell-like diffuse large B-cell lymphoma (GCB-DLBCL) or primary mediastinal B-cell lymphoma (PMBL). In certain embodiments, the cancer is activated B-cell-like diffuse large B-cell lymphoma (ABC-DLBCL). In certain embodiments, the cancer is a hematological cancer.

In certain embodiments, the proliferative disease is a cancer associated with or dependent on a MALT1 fusion protein (e.g., API2-MALT1). In certain embodiments, the proliferative disease is a cancer associated with dependence on B-cell lymphoma 10 (Bcl10). In certain embodiments, the proliferative disease is a cancer associated with dependence on caspase recruitment domain-containing protein (CARD1). In certain embodiments, the proliferative disease is a cancer associated with dependence on NF-κB. In certain embodiments, the cancer is a hematological malignancy.

Additional exemplary cancers include but are not limited to acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); Burkitt lymphoma; primary intraocular lymphoma; classic Hodgkin lymphoma; biphenotypic acute leukemia; T cell lymphoma; nasal-type T cell lymphoma; enteropathy-type T-cell lymphoma; subcutaneous panniculitis-like T-cell lymphoma; blastic NK-cell lymphora; T-cell prolymphocytic leukemia, and NK-cell leukemia.

In certain embodiments, the cancer is a hematological malignancy. Exemplary hematological malignancies include but are not limited to leukemia, such as acute lymphoblastic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), acute non-lymphocytic leukemia (ANLL), acute promyelocytic leukemia (APL), and acute myelomonocytic leukemia (AMMoL); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; multiple myeloma (MM); heavy chain disease (e.g., alpha chain disease, gamma chain disease, mu chain disease), polycythemia vera, Wilm's tumor, and Ewing's sarcoma.

In certain embodiments, said disease or condition mediated by MALT1 is a multiple myeloma. In certain embodiments, said disease or condition mediated by MALT1 is a leukemia (e.g., acute lymphocytic leukemia, acute and chronic myelogenous leukemia, chronic lymphocytic leukemia, acute lymphoblastic leukemia, chronic myelomonocytic leukemia, or promyelocytic leukemia).

In certain embodiments, said disease or condition mediated by MALT1 is a lymphoma (e.g., B-cell lymphoma, T-cell lymphoma, mantle cell lymphoma, diffuse large B cell lymphoma, hairy cell lymphoma, Burkitt's lymphoma, mast cell tumors, Hodgkin's disease or non-Hodgkin's disease). In certain embodiments, said disease or condition mediated by MALT1 is myelodysplastic syndrome. In certain embodiments, said disease or condition mediated by MALT1 is fibrosarcoma. In certain embodiments, said disease or condition mediated by MALT1 is rhabdomyosarcoma. In certain embodiments, said disease or condition mediated by MALT1 is astrocytoma. In certain embodiments, said disease or condition mediated by MALT1 is neuroblastoma. In certain embodiments, said disease or condition mediated by MALT1 is glioma and schwannomas. In certain embodiments, said disease or condition mediated by MALT1 is melanoma. In certain embodiments, said disease or condition mediated by MALT1 is seminoma. In certain embodiments, said disease or condition mediated by MALT1 is teratocarcinoma. In certain embodiments, said disease or condition mediated by MALT1 is osteosarcoma. In certain embodiments, said disease or condition mediated by MALT1 is xenoderma pigmentosum. In certain embodiments, said disease or condition mediated by MALT1 is keratoctanthoma. In certain embodiments, said disease or condition mediated by MALT1 is thyroid follicular cancer. In certain embodiments, said disease or condition mediated by MALT1 is Kaposi's sarcoma. In certain embodiments, said disease or condition mediated by MALT1 is melanoma. In certain embodiments, said disease or condition mediated by MALT1 is teratoma. In certain embodiments, said disease or condition mediated by MALT1 is rhabdomyosarcoma. In certain embodiments, said disease or condition mediated by MALT1 is a metastatic and bone disorder. In certain embodiments, said disease or condition mediated by MALT1 is cancer of the bone. In certain embodiments, said disease or condition mediated by MALT1 is cancer of the mouth/pharynx. In certain embodiments, said disease or condition mediated by MALT1 is cancer of the esophagus. In certain embodiments, said disease or condition mediated by MALT1 is cancer of the larynx. In certain embodiments, said disease or condition mediated by MALT1 is cancer of the stomach. In certain embodiments, said disease or condition mediated by MALT1 is cancer of the intestine. In certain embodiments, said disease or condition mediated by MALT1 is cancer of the colon. In certain embodiments, said disease or condition mediated by MALT1 is cancer of the rectum. In certain embodiments, said disease or condition mediated by MALT1 is cancer of the lung (e.g., non-small cell lung cancer or small cell lung cancer). In certain embodiments, said disease or condition mediated by MALT1 is cancer of the liver. In certain embodiments, said disease or condition mediated by MALT1 is cancer of the pancreas. In certain embodiments, said disease or condition mediated by MALT1 is cancer of the nerve. In certain embodiments, said disease or condition mediated by MALT1 is cancer of the brain (e.g., glioma or glioblastoma multiforme). In certain embodiments, said disease or condition mediated by MALT1 is cancer of the head and neck. In certain embodiments, said disease or condition mediated by MALT1 is cancer of the throat. In certain embodiments, said disease or condition mediated by MALT1 is cancer of the ovary. In certain embodiments, said disease or condition mediated by MALT1 is cancer of the uterus. In certain embodiments, said disease or condition mediated by MALT1 is cancer of the prostate. In certain embodiments, said disease or condition mediated by MALT1 is cancer of the testis. In certain embodiments, said disease or condition mediated by MALT1 is cancer of the bladder. In certain embodiments, said disease or condition mediated by MALT1 is cancer of the kidney. In certain embodiments, said disease or condition mediated by MALT1 is cancer of the breast. In certain embodiments, said disease or condition mediated by MALT1 is cancer of the gall bladder. In certain embodiments, said disease or condition mediated by MALT1 is cancer of the cervix. In certain embodiments, said disease or condition mediated by MALT1 is cancer of the thyroid. In certain embodiments, said disease or condition mediated by MALT1 is cancer of the prostate. In certain embodiments, said disease or condition mediated by MALT1 is cancer of the skin (e.g., skin squamous cell carcinoma). In certain embodiments, said disease or condition mediated by MALT1 is a solid tumor. In certain embodiments, said disease or condition mediated by MALT1 is gastric cancer. In certain embodiments, said disease or condition mediated by MALT1 is hepatocellular carcinoma. In certain embodiments, said disease or condition mediated by MALT1 is a peripheral nerve sheath tumor. In certain embodiments, said disease or condition mediated by MALT1 is pulmonary arterial hypertension.

In certain embodiments, the disease is a cancer associated with a viral infection. In certain embodiments, the disease is a cancer resulting from infection with an oncovirus. In certain embodiments, the oncovirus is hepatitis A, hepatitis B, hepatitis C, human T-lymphotropic virus (HTLV), human papillomavirus (HPV), Kaposi's sarcoma-associated herpesvirus (HHV-8), Merkel cell polyomavirus, or Epstein-Barr virus (EBV). In certain embodiments, the disease is human T-lymphotropic virus. In certain embodiments, the disease is Kaposi's sarcoma-associated herpesvirus. In certain embodiments, the disease is Epstein-Barr virus. Leukemias and lymphomas which may be associated with an oncoviral include: for HTLV, adult T-cell leukemia; for HHV-8, Castleman's disease and primary effusion lymphoma; and for EBV, Burkitt's lymphoma, Hogdkin's lymphoma, and post-transplant lymphoproliferative disease.

In certain embodiments, said disease or condition mediated by MALT1 is an inflammatory disorder or allergic disorder. In certain embodiments, said disease or condition mediated by MALT1 is an inflammatory disorder, such as autoimmune disorders, chronic inflammatory disorders, acute inflammatory disorders, auto-inflammatory disorders, fibrotic disorders, metabolic disorders, neoplasias, cardiovascular or cerebrovascular disorders, and myeloid cell-driven hyper-inflammatory response in COVID-19 infections. In certain embodiments, said disease or condition mediated by MALT1 is an allergic disorder, such as asthma and allergic rhinitis.

In certain embodiments, said disease or condition mediated by MALT1 is a disease or disorder of tissues and systemic disease [e.g., systemic lupus erythematosus (SLE); immune thrombocytopenic purpura (ITP); autoimmune hemolytic anemia (AHA); autoimtnune neutropenia (AlN); Evans syndrome; proliferative and hyperproliferative diseases, such as cancer, atherosclerosis, rheumatoid arthritis, psoriasis, idiopathic pulmonary fibrosis, scleroderma, cirrhosis of the liver; and Acquired Immunodeficiency Syndrome (AIDS)]. In certain embodiments, said disease or condition mediated by MALT1 is an immunologically-mediated disease, such as allograft rejection (e.g., rejection of transplanted organs or tissues). In certain embodiments, said disease or condition mediated by MALT1 is a tissue injury (e.g., associated with organ transplant or revascularization procedures). In certain embodiments, said disease or condition mediated by MALT1 is a disease or disorder of the respiratory tract (e.g., asthma). In certain embodiments, said disease or condition mediated by MALT1 is allergic rhinitis. In certain embodiments, said disease or condition mediated by MALT1 is a disease or disorder of the bone and joints (e.g., arthritis, rheumatoid arthritis). In certain embodiments, said disease or condition mediated by MALT1 is a disease or disorder of the skin. In certain embodiments, said disease or condition mediated by MALT1 is a disease or disorder of the gastrointestinal tract.

In certain embodiments, said disease or condition mediated by MALT1 is a reversible obstructive airways disease, such as asthma (e.g., bronchial asthma, allergic asthma, intrinsic asthma, extrinsic asthma, and dust asthma). In certain embodiments, said disease or condition mediated by MALT1 is chronic or inveterate asthma (e.g., late asthma airways hyper-responsiveness). In certain embodiments, said disease or condition mediated by MALT1 is bronchitis. In certain embodiments, said disease or condition mediated by MALT1 is a condition characterized by an inflammation of the nasal mucus membrane. In certain embodiments, said disease or condition mediated by MALT1 is acute rhinitis. In certain embodiments, said disease or condition mediated by MALT1 is allergic rhinitis. In certain embodiments, said disease or condition mediated by MALT1 is atrophic rhinitis. In certain embodiments, said disease or condition mediated by MALT1 is chronic rhinitis (e.g., rhinitis caseosa, hypertrophic rhinitis, rhinitis purulenta, rhinitis sicca, and rhinitis medicamentosa). In certain embodiments, said disease or condition mediated by MALT1 is membranous rhinitis (e.g., croupous rhinitis, fibrinous rhinitis, pseudomembranous rhinitis, and scrofoulous rhinitis). In certain embodiments, said disease or condition mediated by MALT1 is seasonal rhinitis [e.g., rhinitis nervosa (hay fever), vasomotor rhinitis, sarcoidosis, farmer's lung, and related diseases, such as fibroid lung and idiopathic interstitial pneumonia].

In certain embodiments, said disease or condition mediated by MALT1 includes pannus formation. In certain embodiments, said disease or condition mediated by MALT1 does not include pannus formation. In certain embodiments, said disease or condition mediated by MALT1 is rheumatoid arthritis. In certain embodiments, said disease or condition mediated by MALT1 is seronegative spondyloarthropathis (e.g., ankylosing spondylitis, psoriatic arthritis, and Reiter's disease). In certain embodiments, said disease or condition mediated by MALT1 is Behcet's disease. In certain embodiments, said disease or condition mediated by MALT1 is Sjogren's syndrome. In certain embodiments, said disease or condition mediated by MALT1 is systemic sclerosis.

In certain embodiments, said disease or condition mediated by MALT1 is psoriasis. In certain embodiments, said disease or condition mediated by MALT1 is systemic sclerosis. In certain embodiments, said disease or condition mediated by MALT1 is atopical dermatitis. In certain embodiments, said disease or condition mediated by MALT1 is contact dermatitis. In certain embodiments, said disease or condition mediated by MALT1 is eczematous dermatitis. In certain embodiments, said disease or condition mediated by MALT1 is seborrhoetic dermatitis. In certain embodiments, said disease or condition mediated by MALT1 is Lichen planus. In certain embodiments, said disease or condition mediated by MALT1 is Pemphigus. In certain embodiments, said disease or condition mediated by MALT1 is bullous Pemphigus. In certain embodiments, said disease or condition mediated by MALT1 is epidermolysis bullosa. In certain embodiments, said disease or condition mediated by MALT1 is urticaria. In certain embodiments, said disease or condition mediated by MALT1 is angiodermas. In certain embodiments, said disease or condition mediated by MALT1 is vasculitides. In certain embodiments, said disease or condition mediated by MALT1 is erythemas. In certain embodiments, said disease or condition mediated by MALT1 is cutaneous eosinophilias. In certain embodiments, said disease or condition mediated by MALT1 is uveitis. In certain embodiments, said disease or condition mediated by MALT1 is Alopecia. In certain embodiments, said disease or condition mediated by MALT1 is areata. In certain embodiments, said disease or condition mediated by MALT1 is vernal conjunctivitis.

In certain embodiments, said disease or condition mediated by MALT1 is Coeliac disease. In certain embodiments, said disease or condition mediated by MALT1 is proctitis. In certain embodiments, said disease or condition mediated by MALT1 is eosinophilic gastro-enteritis. In certain embodiments, said disease or condition mediated by MALT1 is mastocytosis. In certain embodiments, said disease or condition mediated by MALT1 is pancreatitis. In certain embodiments, said disease or condition mediated by MALT1 is Crohn's disease. In certain embodiments, said disease or condition mediated by MALT1 is ulcerative colitis. In certain embodiments, said disease or condition mediated by MALT1 is a food-related allergy having effects remote from the gut (e.g., migraine, rhinitis, and eczema).

In certain embodiments, said disease or condition mediated by MALT1 is multiple sclerosis. In certain embodiments, said disease or condition mediated by MALT1 is artherosclerosis. In certain embodiments, said disease or condition mediated by MALT1 is acquired immunodeficiency syndrome (AIDS). In certain embodiments, said disease or condition mediated by MALT1 is lupus. In certain embodiments, said disease or condition mediated by MALT1 is lupus erythematosus. In certain embodiments, said disease or condition mediated by MALT1 is systemic lupus erythematosus. In certain embodiments, said disease or condition mediated by MALT1 is Hashimoto's thyroiditis. In certain embodiments, said disease or condition mediated by MALT1 is myasthenia gravis. In certain embodiments, said disease or condition mediated by MALT1 is type I diabetes. In certain embodiments, said disease or condition mediated by MALT1 is nephrotic syndrome. In certain embodiments, said disease or condition mediated by MALT1 is eosinophilia fasciitis. In certain embodiments, said disease or condition mediated by MALT1 is hyper IgE syndrome. In certain embodiments, said disease or condition mediated by MALT1 is lepromatous leprosy. In certain embodiments, said disease or condition mediated by MALT1 is sezary syndrome. In certain embodiments, said disease or condition mediated by MALT1 is idiopathic thrombocytopenia purpura. In certain embodiments, said disease or condition mediated by MALT1 is restenosis following angioplasty. In certain embodiments, said disease or condition mediated by MALT1 is a tumor (e.g., leukemia, lymphomas). In certain embodiments, said disease or condition mediated by MALT1 is artherosclerosis.

In certain embodiments, said disease or condition mediated by MALT1 is acute chronic allograft rejection (e.g., following transplantation of kidney, heart, liver, lung, bone marrow, skin, or cornea). In certain embodiments, said disease or condition mediated by MALT1 is chronic allograft rejection (e.g., following transplantation of kidney, heart, liver, lung, bone marrow, skin, or cornea). In certain embodiments, said disease or condition mediated by MALT1 is chronic graft-versus-host disease.

In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a fibrotic disorder. In certain embodiments, said disease or condition mediated by MALT1 is a metabolic disorder. In certain embodiments, said disease or condition mediated by MALT1 is a neoplasia. In certain embodiments, said disease or condition mediated by MALT1 is a cardiovascular or cerebrovascular disorder. In certain embodiments, said disease or condition mediated by MALT1 is a myeloid cell-driven hyper-inflammatory response in COVID-19 infections.

In certain embodiments, said disease or condition mediated by MALT1 is an autoimmune disorder. In certain embodiments, said disease or condition mediated by MALT1 is a chronic inflammatory disorder. In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a combination of one, two, or all three of a chronic inflammatory disorder, an acute inflammatory disorder, and an auto-inflammatory disorder.

In certain embodiments, said disease or condition mediated by MALT1 is an inflammatory bowel disease (e.g., ulcerative colitis or Crohn's disease). In certain embodiments, said disease or condition mediated by MALT1 is multiple sclerosis. In certain embodiments, said disease or condition mediated by MALT1 is psoriasis. In certain embodiments, said disease or condition mediated by MALT1 is arthritis. In certain embodiments, said disease or condition mediated by MALT1 is rheumatoid arthritis. In certain embodiments, said disease or condition mediated by MALT1 is osteoarthritis. In certain embodiments, said disease or condition mediated by MALT1 is juvenile arthritis. In certain embodiments, said disease or condition mediated by MALT1 is psoriatic arthritis. In certain embodiments, said disease or condition mediated by MALT1 is reactive arthritis. In certain embodiments, said disease or condition mediated by MALT1 is ankylosing spondylitis. In certain embodiments, said disease or condition mediated by MALT1 is cryopyrin-associated periodic syndromes. In certain embodiments, said disease or condition mediated by MALT1 is Muckle-Wells syndrome. In certain embodiments, said disease or condition mediated by MALT1 is familial cold auto-inflammatory syndrome. In certain embodiments, said disease or condition mediated by MALT1 is neonatal-onset multisystem inflammatory disease. In certain embodiments, said disease or condition mediated by MALT1 is TNF receptor-associated periodic syndrome. In certain embodiments, said disease or condition mediated by MALT1 is acute and chronic pancreatitis. In certain embodiments, said disease or condition mediated by MALT1 is atherosclerosis. In certain embodiments, said disease or condition mediated by MALT1 is gout. In certain embodiments, said disease or condition mediated by MALT1 is a fibrotic disorder (e.g., hepatic fibrosis or idiopathic pulmonary fibrosis). In certain embodiments, said disease or condition mediated by MALT1 is nephropathy. In certain embodiments, said disease or condition mediated by MALT1 is sarcoidosis. In certain embodiments, said disease or condition mediated by MALT1 is scleroderma. In certain embodiments, said disease or condition mediated by MALT1 is anaphylaxis. In certain embodiments, said disease or condition mediated by MALT1 is diabetes (e.g., diabetes mellitus type 1 or diabetes mellitus type 2). In certain embodiments, said disease or condition mediated by MALT1 is diabetic retinopathy. In certain embodiments, said disease or condition mediated by MALT1 is Still's disease. In certain embodiments, said disease or condition mediated by MALT1 is vasculitis. In certain embodiments, said disease or condition mediated by MALT1 is sarcoidosis. In certain embodiments, said disease or condition mediated by MALT1 is pulmonary inflammation. In certain embodiments, said disease or condition mediated by MALT1 is respiratory failure. In certain embodiments, said disease or condition mediated by MALT1 is acute respiratory distress syndrome. In certain embodiments, said disease or condition mediated by MALT1 is chronic eosinophilic pneumonia. In certain embodiments, said disease or condition mediated by MALT1 is wet and dry age-related macular degeneration. In certain embodiments, said disease or condition mediated by MALT1 is autoimmune hemolytic syndromes. In certain embodiments, said disease or condition mediated by MALT1 is autoimmune and inflammatory hepatitis. In certain embodiments, said disease or condition mediated by MALT1 is autoimmune neuropathy. In certain embodiments, said disease or condition mediated by MALT1 is autoimmune ovarian failure. In certain embodiments, said disease or condition mediated by MALT1 is autoimmune orchitis. In certain embodiments, said disease or condition mediated by MALT1 is autoimmune thrombocytopenia. In certain embodiments, said disease or condition mediated by MALT1 is silicone implant-associated autoimmune disease. In certain embodiments, said disease or condition mediated by MALT1 is Sjogren's syndrome. In certain embodiments, said disease or condition mediated by MALT1 is familial Mediterranean fever. In certain embodiments, said disease or condition mediated by MALT1 is systemic lupus erythematosus. In certain embodiments, said disease or condition mediated by MALT1 is vasculitis syndromes (e.g., t. emporal, Takayasu's and giant cell arteritis, Behcet's disease or Wegener's granulomatosis). In certain embodiments, said disease or condition mediated by MALT1 is vitiligo. In certain embodiments, said disease or condition mediated by MALT1 is secondary hematologic manifestation of autoimmune diseases (e.g., anemias). In certain embodiments, said disease or condition mediated by MALT1 is drug-induced autoimmunity. In certain embodiments, said disease or condition mediated by MALT1 is Hashimoto's thyroiditis. In certain embodiments, said disease or condition mediated by MALT1 is hypophysitis. In certain embodiments, said disease or condition mediated by MALT1 is idiopathic thrombocytic pupura. In certain embodiments, said disease or condition mediated by MALT1 is metal-induced autoimmunity. In certain embodiments, said disease or condition mediated by MALT1 is myasthenia gravis. In certain embodiments, said disease or condition mediated by MALT1 is pemphigus. In certain embodiments, said disease or condition mediated by MALT1 is autoimmune deafness (e.g., Meniere's disease). In certain embodiments, said disease or condition mediated by MALT1 is Goodpasture's syndrome. In certain embodiments, said disease or condition mediated by MALT1 is Graves' disease. In certain embodiments, said disease or condition mediated by MALT1 is an HW-related autoimmune syndromes. In certain embodiments, said disease or condition mediated by MALT1 is Gullain-Barre disease. In certain embodiments, said disease or condition mediated by MALT1 is Addison's disease. In certain embodiments, said disease or condition mediated by MALT1 is anti-phospholipid syndrome. In certain embodiments, said disease or condition mediated by MALT1 is asthma. In certain embodiments, said disease or condition mediated by MALT1 is atopic dermatitis. In certain embodiments, said disease or condition mediated by MALT1 is Celiac disease. In certain embodiments, said disease or condition mediated by MALT1 is Cushing's syndrome. In certain embodiments, said disease or condition mediated by MALT1 is dermatomyositis. In certain embodiments, said disease or condition mediated by MALT1 is idiopathic adrenal atrophy. In certain embodiments, said disease or condition mediated by MALT1 is idiopathic thrombocytopenia. In certain embodiments, said disease or condition mediated by MALT1 is Kawasaki syndrome. In certain embodiments, said disease or condition mediated by MALT1 is Lambert-Eaton Syndrome. In certain embodiments, said disease or condition mediated by MALT1 is pernicious anemia. In certain embodiments, said disease or condition mediated by MALT1 is pollinosis. In certain embodiments, said disease or condition mediated by MALT1 is polyarteritis nodosa. In certain embodiments, said disease or condition mediated by MALT1 is primary biliary cirrhosis. In certain embodiments, said disease or condition mediated by MALT1 is primary sclerosing cholangitis. In certain embodiments, said disease or condition mediated by MALT1 is Raynaud's disease. In certain embodiments, said disease or condition mediated by MALT1 is Raynaud's phenomenon. In certain embodiments, said disease or condition mediated by MALT1 is Reiter's Syndrome. In certain embodiments, said disease or condition mediated by MALT1 is relapsing polychondritis. In certain embodiments, said disease or condition mediated by MALT1 is Schmidt's syndrome. In certain embodiments, said disease or condition mediated by MALT1 is thyrotoxidosis. In certain embodiments, said disease or condition mediated by MALT1 is sepsis. In certain embodiments, said disease or condition mediated by MALT1 is septic shock. In certain embodiments, said disease or condition mediated by MALT1 is endotoxic shock. In certain embodiments, said disease or condition mediated by MALT1 is exotoxin-induced toxic shock. In certain embodiments, said disease or condition mediated by MALT1 is gram negative sepsis. In certain embodiments, said disease or condition mediated by MALT1 is toxic shock syndrome. In certain embodiments, said disease or condition mediated by MALT1 is glomerulonephritis. In certain embodiments, said disease or condition mediated by MALT1 is peritonitis. In certain embodiments, said disease or condition mediated by MALT1 is interstitial cystitis. In certain embodiments, said disease or condition mediated by MALT1 is hyperoxia-induced inflammations. In certain embodiments, said disease or condition mediated by MALT1 is chronic obstructive pulmonary disease (COPD). In certain embodiments, said disease or condition mediated by MALT1 is emphysema. In certain embodiments, said disease or condition mediated by MALT1 is nasal inflammation. In certain embodiments, said disease or condition mediated by MALT1 is vasculitis. In certain embodiments, said disease or condition mediated by MALT1 is graft vs. host reaction (e.g., graft vs. host disease). In certain embodiments, said disease or condition mediated by MALT1 is allograft rejections (e.g., acute allograft rejection or chronic allograft rejection). In certain embodiments, said disease or condition mediated by MALT1 is early transplantation rejection (e.g., acute allograft rejection). In certain embodiments, said disease or condition mediated by MALT1 is reperfusion injury. In certain embodiments, said disease or condition mediated by MALT1 is pain (e.g., acute pain, chronic pain, neuropathic pain, or fibromyalgia). In certain embodiments, said disease or condition mediated by MALT1 is a chronic infection. In certain embodiments, said disease or condition mediated by MALT1 is meningitis. In certain embodiments, said disease or condition mediated by MALT1 is encephalitis. In certain embodiments, said disease or condition mediated by MALT1 is myocarditis. In certain embodiments, said disease or condition mediated by MALT1 is gingivitis. In certain embodiments, said disease or condition mediated by MALT1 is post-surgical trauma. In certain embodiments, said disease or condition mediated by MALT1 is tissue injury. In certain embodiments, said disease or condition mediated by MALT1 is traumatic brain injury. In certain embodiments, said disease or condition mediated by MALT1 is enterocolitis. In certain embodiments, said disease or condition mediated by MALT1 is sinusitis. In certain embodiments, said disease or condition mediated by MALT1 is uveitis. In certain embodiments, said disease or condition mediated by MALT1 is ocular inflammation. In certain embodiments, said disease or condition mediated by MALT1 is optic neuritis. In certain embodiments, said disease or condition mediated by MALT1 is gastric ulcers. In certain embodiments, said disease or condition mediated by MALT1 is esophagitis. In certain embodiments, said disease or condition mediated by MALT1 is peritonitis. In certain embodiments, said disease or condition mediated by MALT1 is periodontitis. In certain embodiments, said disease or condition mediated by MALT1 is dermatomyositis. In certain embodiments, said disease or condition mediated by MALT1 is gastritis. In certain embodiments, said disease or condition mediated by MALT1 is myositis. In certain embodiments, said disease or condition mediated by MALT1 is polymyalgia. In certain embodiments, said disease or condition mediated by MALT1 is pneumonia. In certain embodiments, said disease or condition mediated by MALT1 is bronchitis. In certain embodiments, the disease or condition mediated by MALT1 is endometriosis. In certain embodiments, the disease or condition mediated by MALT1 is necrotizing vasculitis. In certain embodiments, the disease or condition mediated by MALT1 is lymphadenitis. In certain embodiments, the disease or condition mediated by MALT1 is peri-arteritis nodosa. In certain embodiments, the disease or condition mediated by MALT1 is anti-phospholipid antibody syndrome. In certain embodiments, the disease or condition mediated by MALT1 is pemphigus vulgaris. In certain embodiments, the disease or condition mediated by MALT1 is Lyme disease. In certain embodiments, the disease or condition mediated by MALT1 is cardiomyopathy. In certain embodiments, the disease or condition mediated by MALT1 isrheumatic fever. In certain embodiments, the disease or condition mediated by MALT1 is a blistering disorder. In certain embodiments, the disease or condition mediated by MALT1 is an antibody-mediated vasculitis syndrome. In certain embodiments, the disease or condition mediated by MALT1 is an immune-complex vasculitide. In certain embodiments, the disease or condition mediated by MALT1 is oedema. In certain embodiments, the disease or condition mediated by MALT1 is embolism. In certain embodiments, the disease or condition mediated by MALT1 is fibrosis. In certain embodiments, the disease or condition mediated by MALT1 is silicosis. In certain embodiments, the disease or condition mediated by MALT1 is BENTA disease. In certain embodiments, the disease or condition mediated by MALT1 is berylliosis.

In certain embodiments, said disease or condition mediated by MALT1 is systemic sclerosis/scleroderma. In certain embodiments, said disease or condition mediated by MALT1 is lupus nephritis. In certain embodiments, said disease or condition mediated by MALT1 is connective tissue disease. In certain embodiments, said disease or condition mediated by MALT1 is wound healing. In certain embodiments, said disease or condition mediated by MALT1 is surgical scarring. In certain embodiments, said disease or condition mediated by MALT1 is spinal cord injury. In certain embodiments, said disease or condition mediated by MALT1 is CNS scarring. In certain embodiments, said disease or condition mediated by MALT1 is acute lung injury. In certain embodiments, said disease or condition mediated by MALT1 is pulmonary fibrosis (e.g., idiopathic pulmonary fibrosis or cystic fibrosis). In certain embodiments, said disease or condition mediated by MALT1 is chronic obstructive pulmonary disease. In certain embodiments, said disease or condition mediated by MALT1 is adult respiratory distress syndrome. In certain embodiments, said disease or condition mediated by MALT1 is acute lung injury. In certain embodiments, said disease or condition mediated by MALT1 is drug-induced lung injury. In certain embodiments, said disease or condition mediated by MALT1 is glomerulonephritis. In certain embodiments, said disease or condition mediated by MALT1 is chronic kidney disease (e.g., diabetic nephropathy). In certain embodiments, said disease or condition mediated by MALT1 is hypertension-induced nephropathy. In certain embodiments, said disease or condition mediated by MALT1 is alimentary track or gastrointestinal fibrosis. In certain embodiments, said disease or condition mediated by MALT1 is renal fibrosis. In certain embodiments, said disease or condition mediated by MALT1 is hepatic or biliary fibrosis. In certain embodiments, said disease or condition mediated by MALT1 is liver fibrosis (e.g., nonalcoholic steatohepatitis, hepatitis C, or hepatocellular carcinoma). In certain embodiments, said disease or condition mediated by MALT1 is cirrhosis (e.g., primary biliary cirrhosis or cirrhosis due to fatty liver disease, such as alcoholic and nonalcoholic steatosis). In certain embodiments, said disease or condition mediated by MALT1 is radiation-induced fibrosis (e.g., head and neck, gastrointestinal or pulmonary). In certain embodiments, said disease or condition mediated by MALT1 is primary sclerosing cholangitis. In certain embodiments, said disease or condition mediated by MALT1 is restenosis. In certain embodiments, said disease or condition mediated by MALT1 is cardiac fibrosis (e.g., endomyocardial fibrosis or atrial fibrosis). In certain embodiments, said disease or condition mediated by MALT1 is opthalmic scarring. In certain embodiments, said disease or condition mediated by MALT1 is fibrosclerosis. In certain embodiments, said disease or condition mediated by MALT1 is a fibrotic cancer. In certain embodiments, said disease or condition mediated by MALT1 is fibroids. In certain embodiments, said disease or condition mediated by MALT1 is fibroma. In certain embodiments, said disease or condition mediated by MALT1 is a fibroadenoma. In certain embodiments, said disease or condition mediated by MALT1 is a fibrosarcoma. In certain embodiments, said disease or condition mediated by MALT1 is transplant arteriopathy. In certain embodiments, said disease or condition mediated by MALT1 is keloid. In certain embodiments, said disease or condition mediated by MALT1 is mediastinal fibrosis. In certain embodiments, said disease or condition mediated by MALT1 is myelofibrosis. In certain embodiments, said disease or condition mediated by MALT1 is retroperitoneal fibrosis. In certain embodiments, said disease or condition mediated by MALT1 is progressive massive fibrosis. In certain embodiments, said disease or condition mediated by MALT1 is nephrogenic systemic fibrosis.

In certain embodiments, said disease or condition mediated by MALT1 is obesity. In certain embodiments, said disease or condition mediated by MALT1 is steroid-resistance. In certain embodiments, said disease or condition mediated by MALT1 is glucose intolerance. In certain embodiments, said disease or condition mediated by MALT1 is metabolic syndrome.

In certain embodiments, said disease or condition mediated by MALT1 is atherosclerosis. In certain embodiments, said disease or condition mediated by MALT1 is restenosis of an atherosclerotic coronary artery. In certain embodiments, said disease or condition mediated by MALT1 is acute coronary syndrome. In certain embodiments, said disease or condition mediated by MALT1 is myocardial infarction. In certain embodiments, said disease or condition mediated by MALT1 is cardiac-allograft vasculopathy. In certain embodiments, said disease or condition mediated by MALT1 is stroke. In certain embodiments, said disease or condition mediated by MALT1 is a central nervous system disorder with an inflammatory or apoptotic component. In certain embodiments, said disease or condition mediated by MALT1 is Alzheimer's disease. In certain embodiments, said disease or condition mediated by MALT1 is Parkinson's disease. In certain embodiments, said disease or condition mediated by MALT1 is Huntington's disease. In certain embodiments, said disease or condition mediated by MALT1 is amyotrophic lateral sclerosis. In certain embodiments, said disease or condition mediated by MALT1 is spinal cord injury. In certain embodiments, said disease or condition mediated by MALT1 is neuronal ischemia. In certain embodiments, said disease or condition mediated by MALT1 is peripheral neuropathy.

In certain embodiments, said disease or condition mediated by MALT1 is a disease or disorder associated with a coronavirus (e.g., SARS-CoV-2). In certain embodiments, said coronavirus is SARS-CoV-2. In certain embodiments, the disease or disorder associated with SARS-CoV-2 is COVID-19.

In certain embodiments, the disease or condition mediated by MALT1 is a rheumatic disease. In certain embodiments, the disease or condition mediated by MALT1 is an inflammatory arthropathy. In certain embodiments, the disease or condition mediated by MALT1 is rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile Idiopathic Arthritis (JIA), juvenile psoriatic arthritis, juvenile rheumatoid arthritis, systemic onset juvenile rheumatoid arthritis, giant cell arteritis, secondary osteoarthritis from an inflammatory disease.

In certain embodiments, the disease or condition mediated by MALT1 is a connective tissue disease. In certain embodiments, the disease or condition mediated by MALT1 is lupus, systemic lupus erythematosus, juvenile systemic lupus erythematosus, nephritis, Sjögren's syndrome, scleroderma (systemic sclerosis), Raynaud's phenomenonjuvenile scleroderma, polymyositis, dermatomyositis, polymyositis-dermatomyositis, polymyalgia rheumatica, a mixed connective tissue disease, sarcoidosis, fibromyalgia, vasculitis microscopic polyangiitis, vasculitis, eosinophilic granulomatosis with polyangiitis (formerly known as Churg-Strauss Syndrome), granulomatosis with polyangiitis (formerly known as Wegener's granulomatosis), polyarteritis nodosa, Henoch-Schonlein purpura, idiopathic thrombocytopenic thrombotic purpura, juvenile vasculitis, polyarteritis nodossa (also known as panarteritis nodosa, periarteritis nodosa Kussmaul disease, Kussmaul-Maier disease or PAN), serum sickness, myasthenia gravis, Takayasu's arteritis, Behget's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, primary biliary sclerosis, autoimmune hepatitis, chronic aggressive hepatitis, nonalcoholic hepatic steatosis, sclerosing cholangitis, membranous glomerulopathy, polymyositis, myositis, atherosclerosis, autoimmune hemolytic anemia, autoimmune orchitis, Goodpasture's disease,

In certain embodiments, the disease or condition mediated by MALT1 is a neurodegenerative disease or neuroinflammatory disease. In certain embodiments, the disease or condition mediated by MALT1 is multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder.

In certain embodiments, the disease or condition mediated by MALT1 is an inflammatory bowel disease. In certain embodiments, the disease or condition mediated by MALT1 is Crohn's disease, ulcerative colitis, Celiac Sprue, Celiac disease, proctitis, eosinophilic gastroenteritis, autoimmune atrophic gastritis of pernicious anemia, or mastocytosis.

In certain embodiments, the disease or condition mediated by MALT1 is a skin autoimmune disorder. In certain embodiments, the disease or condition mediated by MALT1 is psoriasis. In certain embodiments, the disease or condition mediated by MALT1 is eczema. In certain embodiments, the disease or condition mediated by MALT1 is plaque psoriasis, Guttate psoriasis, psoriatic epidermal hyperplasia, inverse psoriasis, pustular psoriasis, erythrodermic psoriasis, atopic dermatitis, eczema dermatitis, dermatitis, rosacea, pruritus, alopecia areata, vitiligo, epidermal hyperplasia, juvenile dermatomyositis, dermatomyositis, or hidradenitis suppurativa.

In certain embodiments, the disease or condition mediated by MALT1 is an organ or cell transplant rejection. In certain embodiments, the disease or condition mediated by MALT1 is graft-versus-host disease. In certain embodiments, the disease or condition mediated by MALT1 is chronic graft-versus-host disease, acute graft-versus-host disease, or organ or cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In certain embodiments, the disease or condition mediated by MALT1 is an autoimmune disease of the eye. In certain embodiments, the disease or condition mediated by MALT1 is Graves' disease, noninfectious uveitis, dry eye syndrome, sympathetic ophthalmia, Cogan's syndrome, keratoconjunctivitis, vernal conjunctivitis, uveitis (e.g., uveitis associated with Behcet's disease and lens-induced uveitis), keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, or ocular neovascularization

In certain embodiments, the disease or condition mediated by MALT1 is an ocular manifestation of an autoimmune disease.

In certain embodiments, the disease or condition mediated by MALT1 is a respiratory disease. In certain embodiments, the disease or condition mediated by MALT1 is asthma, chronic obstructive pulmonary disease, or acute respiratory disease.

In certain embodiments, the disease or condition mediated by MALT1 is diabetes. In certain embodiments, the disease or condition mediated by MALT1 is Type I diabetes mellitus, Type II diabetes mellitus, or juvenile onset diabetes.

### Additional Methods

Another aspect of the invention provides methods of inhibiting cell proliferation in a subject by administering to the subject a compound described herein (e.g., a compound of Formula I, I-1, I-2, I-3, I-4, or II, or other compounds in Section I), or inhibiting cell proliferation in a biological sample by contacting the biological sample with a compound described herein (e.g., a compound of Formula I, I-1, I-2, I-3, I-4, or II, or other compounds in Section I). In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above. In certain embodiments, cell proliferation is inhibited for T-cells. In certain embodiments, cell proliferation is inhibited for B-cells. In certain embodiments, cell proliferation is inhibited for T-cells and B-cells.

Another aspect of the invention provides methods of inducing apoptosis of a cell in a subject by administering to the subject a compound described herein (e.g., a compound of Formula I, I-1, I-2, I-3, I-4, or II, or other compounds in Section I), or inducing apoptosis of a cell in a biological sample by contacting the biological sample with a compound described herein (e.g., a compound of Formula I, I-1, I-2, I-3, I-4, or II, or other compounds in Section I). In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above. In certain embodiments, cell is a tumor cell. In certain embodiments, the cell is a lymphocyte. In certain embodiments, the cell is a T-cell. In certain embodiments, the cell is a B-cell.

Another aspect of the invention provides methods of inhibiting adhesion of a cell in a subject by administering to the subject a compound described herein (e.g., a compound of Formula I, I-1, I-2, I-3, I-4, or II, or other compounds in Section I), or inhibiting adhesion of a cell in a biological sample by contacting the biological sample with a compound described herein (e.g., a compound of Formula I, I-1, I-2, I-3, I-4, or II, or other compounds in Section I). In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above. In certain embodiments, the cell is a tumor cell. In certain embodiments, the cell is a lymphocyte. In certain embodiments, the cell is a T-cell. In certain embodiments, the cell is a B-cell.

Another aspect of the invention provides methods of inhibiting activation of T-cells or B-cells in a subject by administering to the subject a compound described herein (e.g., a compound of Formula I, I-1, I-2, I-3, I-4, or II, or other compounds in Section I), or inhibiting activation of T-cells or B-cells in a biological sample by contacting the biological sample with a compound described herein (e.g., a compound of Formula I, I-1, I-2, I-3, I-4, or II, or other compounds in Section I). In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above.

Another aspect of the invention provides methods of inhibiting the activity of mucosa-associated lymphoid tissue lymphoma translation protein 1 (MALT1) or a MALT1 fusion protein in a subject by administering to the subject a compound described herein (e.g., a compound of Formula I, I-1, I-2, I-3, I-4, or II, or other compounds in Section I), or inhibiting the activity of mucosa-associated lymphoid tissue lymphoma translation protein 1 (MALT1) or a MALT1 fusion protein in a biological sample by contacting the biological sample with a compound described herein (e.g., a compound of Formula I, I-1, I-2, I-3, I-4, or II, or other compounds in Section I). In certain embodiments, the compound is a compound of Formula Ia, Ib, Ic, Id, Ie, If, Ig, Ih, Ii, Ij, Ik, Il, Im, In, Io, Ip, Ia-4, Ib-4, Ic-4, Id-4, Ie-4, If-4, Ig-4, Ih-4, Ii-4, Ij-4, Ik-4, Il-4, Im-4, In-4, Io-4, Ip-4 or defined by by one of the embodiments described above. In certain embodiments, the method inhibits the protease activity of MALT1. In certain embodiments, the method inhibits the protease activity of a MALT1 fusion protein (e.g., API2-MALT1). In certain embodiments, the method inhibits the protease activity of MALT1 or a MALT1 fusion protein for cleavage of a peptide substrate. In certain embodiments, the peptide substrate is A20, Bcl10, RelB, CylD, NIK, regnase-1, roquin-1, roquin-2, LIMA1α, or MALT1. The inhibitor may selectively inhibit the protease activity of MALT1 or a MALT1 fusion protein for cleavage of a first peptide substrate over protease activity for cleavage of a second peptide substrate. In certain embodiments, the first and/or second substrate is A20, Bcl10, RelB, CylD, NIK, regnase-1, roquin-1, roquin-2, LIMAla, or MALT1. In certain embodiments, the selectivity is between about 1.25 fold and about 5 fold. In certain embodiments, the selectivity is between about 5 fold and about 10 fold. In certain embodiments, the selectivity is between about 10 fold and about 25 fold. In certain embodiments, the selectivity is between about 25 fold and about 50 fold. In certain embodiments, the selectivity is between about 50 fold and about 100 fold. In certain embodiments, the selectivity is between about 100 fold and about 250 fold. In certain embodiments. In certain embodiments, the selectivity is between about 250 fold and about 500 fold. In certain embodiments, the selectivity is between about 500 fold and about 1000 fold. In certain embodiments, or at least about 1000 fold.

### III. Combination Therapy

Another aspect of the invention provides for combination therapy. Pyrazolylsulfonamide compounds described herein (e.g., a compound of Formula I, I-1, I-2, I-3, I-4, II, or other compounds in Section I) or their pharmaceutically acceptable salts may be used in combination with additional therapeutic agents to treat diseases or conditions, such as an inflammatory disorder.

Accordingly, in some embodiments, the present invention provides a method of treating a disclosed disease or condition comprising administering to a patient in need thereof an effective amount of a compound disclosed herein and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents.

One or more other therapeutic agents may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another.

In certain embodiments, the compounds of the disclosure can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In certain embodiments, the compound of the disclosure can be administered first followed by a second therapeutic agent, or alternatively, the second therapeutic agent administered first followed by the compound of the disclosure. In certain embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the second therapeutic agent, by the same or different routes, or administered in a single composition by the same route. In certain embodiments, the compound of the disclosure is prepared as a first pharmaceutical composition, and the second therapeutic agent prepared as a second pharmaceutical composition, where the first pharmaceutical composition and the second pharmaceutical composition are administered simultaneously, sequentially, or separately. In certain embodiments, the amount and frequency of administration of the second therapeutic agent can used standard dosages and standard administration frequencies used for the particular therapeutic agent. See, e.g., Physicians' DeskReference, 70^{th}Ed., PDR Network, 2015; incorporated herein by reference.

In certain embodiments, the additional therapeutic agent is a leukotriene inhibitor, non-steroidal anti-inflammatory drug (NSAID), steroid, tyrosine kinase inhibitor, receptor kinase inhibitor, modulator of nuclear receptor family of transcription factor, HSP90 inhibitor, adenosine receptor (A₂A) agonist, disease modifying antirheumatic drugs (DMARDS), phosphodiesterase (PDE) inhibitor, neutrophil elastase inhibitor, modulator of Axl kinase, an anti-cancer agent, anti-allergic agent, anti-nausea agent (or anti-emetic), pain reliever, cytoprotective agent, or a combination thereof. In certain embodiments, the additional therapeutic agent is an anti-cancer agent, an analgesic, an anti-inflammatory agent, or a combination thereof.

In certain embodiments, the second therapeutic agent is a leukotriene inhibitor. Examples of leukotriene inhibitors considered for use in combination therapies of the invention include but are not limited to montelukast, zafirlukast, pranlukast, zileuton, or combinations thereof.

In certain embodiments, the second therapeutic agent is a an NSAID. Examples of NSAIDs considered for use in combination therapies of the invention include but are not limited to acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib, or combinations thereof.

In certain embodiments, the second therapeutic agent is a steroid. Examples of steroids considered for use in combination therapies of the invention include but are not limited to prednisone, prednisolone, methylprednisone, triacmcinolone, betamethasone, dexamethasone, and prodrugs thereof.

In certain embodiments, the second therapeutic agent is a tyrosine kinase inhibitor. Examples of tyrosine kinase inhibitors considered for use in combination therapies of the invention include but are not limited to inhibitors of the following kinases, including, among others: JAK, Syk, JNK/SAPK, MAPK, PI-3K, and/or Ripk2. In certain embodiments, the tyrosine kinase inhibitor is ruxolitinib, tofacitinib, oclactinib, filgotinib, ganotinib, lestaurtinib, momelotinib, pacritinib, upadacitinib, peficitinib, fedratinib, bentamapimod, D-JNKI-1 (XG-102, AM-111), ponatinib, WEHI-345, OD36, GSK583, idelalisib, copanlisib, taselisib, duvelisib, alpelisib, umbralisib, dactolisib, CUDC-907, entospletinib, fostamatinib, or combinations thereof. In certain embodiments, the second therapeutic agent is a Bruton Tyrosine Kinase (BTK) inhibitor. Examples of BTK inhibitors considered for use in combination therapies of the invention include but are not limited to ibrutinib, acalabrutinib, pirtobrutinib, and zanubrutinib.

In certain embodiments, the second therapeutic agent is a receptor kinase inhibitor, including among others, an inhibitor of EGFR or HER2. Examples of receptor kinase inhibitors considered for use in combination therapies of the invention include but are not limited to gefitinib, erlotinib, neratinib, lapatinib, cetuximab, panitumumab, vandetanib, necitumumab, osimertinib, trastuzumab, neratinib, lapatinib, pertuzumab, or combinations thereof.

In certain embodiments, the second therapeutic agent is a modulator of nuclear receptor family of transcription factors, including, among others, an inhibitor of PPAR, RXR, FXR, or LXR. In certain embodiments, the inhibitor is pioglitazone, bexarotene, obeticholic acid, ursodeoxycholic acid, fexaramine, hypocholamide, or combinations thereof.

In certain embodiments, the second therapeutic agent is an HSP90 inhibitor. Examples of HSP90 inhibitors considered for use in combination therapies of the invention include but are not limited to ganetespib, 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010, or combinations thereof.

In certain embodiments, the second therapeutic agent is an adenosine receptor 2A (A₂A) agonist. Examples of adenosine receptor agonists considered for use in combination therapies of the invention include but are not limited to those disclosed in U.S. Pat. No. 9,067,963, which is incorporated herein by reference. In certain embodiments, the adenosine receptor agonist is LNC-3050, LNC-3015, LNC-3047, LNC-3052, or combinations thereof.

In certain embodiments, the second therapeutic agent is selected from disease modifying antirheumatic drugs (DMARDS). Examples of DMARDS considered for use in combination therapies of the invention include but are not limited to tocilizumab, certolizumab, etanercept, adalimumab, anakinra, abatacept, infliximab, rituximab, golimumab, uteskinumab, or combinations thereof.

In certain embodiments, the second therapeutic agent is a phosphodiesterase (PDE) inhibitor. Examples of phosphodiesterase inhibitor considered for use in combination therapies of the invention include but are not limited to apremilast, crisaborole, piclimilast, drotaverine, ibudulast, roflumilast, sildenafil, tadalafil, vardenafil, or combinations thereof.

In certain embodiments, the second therapeutic agent is a neutrophil elastase inhibitor. Examples of neutrophil elastase inhibitors considered for use in combination therapies of the invention include but are not limited to sivelestat.

In certain embodiments, the second therapeutic agent is a modulator of Axl kinase. Examples of modulators of Axl kinase considered for use in combination therapies of the invention include but are not limited to bemcentinib (BGB324 or R428), TP-0903, LY2801653, amuvatinib (MP-470), bosutinib (SKI-606), MGCD 265, ASP2215, cabozantinib (XL184), foretinib (GSK1363089/XL880), and SGI-7079. In certain embodiments, the modulator of Axl kinase is a monoclonal antibody targeting AXL (e.g., YW327.6S2) or an AXL decoy receptor (e.g., GL2I.T), or glesatinib, merestinib, or a dual Flt3-Axl inhibitor such as gilteritinib.

In certain embodiments, the second therapeutic agent is a bispecific antibody, such as a bispecific antibody that binds to a tumor-specific antigen. Exemplary bispecific antibodies include but are not limited to Blincyto (blinatumomab), Kimmtrak (tebentafusp), Tecvayli (teclistamab), Lunsumio (mosunetuzumab), Epkinly (epcoritamab), and Columvi (glofitamab).

In certain embodiments, the second therapeutic agent is a chimeric antigen receptor (CAR) T-cell therapy. Exemplary CAR T-cell therapies include but are not limited to ABECMA® (idecabtagene vicleucel), BREYANZI® (lisocabtagene maraleucel), CARVYKTI™ (ciltacabtagene autoleucel), KYMRIAH™ (tisagenlecleucel), TECARTUS™ (brexucabtagene autoleucel), and YESCARTA™ (axicabtagene ciloleucel).

In certain embodiments, the additional therapeutic agent is an anti-cancer agent or chemo-therapeutic agent. Examples of anti-cancer agents considered for use in combination therapies of the invention include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960,Cea-Vac, BrevaRex AR54, IMC-1C11, GlioMab-H, ING-1, anti-LCG Mabs, MT-103, KSB-303, Therex, KW2871, anti-HMI.24, Anti-PTHrP, 2C4 antibody, SGN-30, TRAIL-RI Mab, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MylERAN®), busulfan injection (BUSULFEX®), capecitabine (XELODA®), N4-pentoxycarbonyl-5-deoxy-5-fluorocytidine, carboplatin (PARAPLATIN®), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCTL®, EFUDEX®), flutamide (EULEXIN®), tezacitibine, gemcitabine (difluorodeoxycitidine), hydroxyurea (hydrEA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MylOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present invention are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, Byl719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON® A), ipilimumab, trametinib (e.g., MEKINIST®), peginterferon alfa-2b (e.g., PEGINTRON®, SylATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOL®, PLATINOL-AQ®), doxorubicin hydrochloride liposome (DOXIL®, DOX-SL®, EVacet®, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL@, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (TRESSA®), and gemcitabine hydrochloride (GEMZAR®).

For the treatment of pancreatic cancer, the other therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATE®, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In some instances, patients may experience allergic reactions to the compounds of the present invention and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, hydrOCORT Acetate® and LANACORT®), prednisolone (e.g., DELTA-Cortel®, ORAPRED®, PEDIAPRED® and PRELONE®), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRyl®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the compound of the present invention and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®. Dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TylENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., oxyCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In yet another aspect, a compound of the present invention may be used in combination with known therapeutic processes, for example, with the administration of hormones or in radiation therapy. In certain instances, a compound of the present invention may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the compound described herein (e.g., a compound of Formula I, I-1, I-2, I-3, I-4, or II, or other compounds in Section I) and the additional therapeutic agent(s) are administered in doses commonly employed when such agents are used as monotherapy for treating the disease or condition. In other embodiments, the compound described herein (e.g., a compound of Formula I, I-1, I-2, I-3, I-4, or II, or other compounds in Section I) and the additional therapeutic agent(s) are administered in doses lower than the doses commonly employed when such agents are used as monotherapy for treating the disease or condition. In certain embodiments, the compound described herein (e.g., a compound of Formula I, I-1, I-2, I-3, I-4, or II, or other compounds in Section I) and the additional therapeutic agent(s) are present in the same composition, which is suitable for oral administration.

In certain embodiments, the compound described herein (e.g., a compound of Formula I, I-1, I-2, I-3, I-4, or II, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I, I-1, I-2, I-3, I-4, or II, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (e.g., capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a imidazopyrimidine compound or related compound described herein in a therapeutically effective amount for the treatment of a disease or condition described herein.

### IV. Medical Kits

Another aspect of the invention provides a medical kit comprising, for example, (i) a compound described herein, and (ii) instructions for use according to a method described herein.

### V. Enumerated Embodiments

The following exemplary embodiments are provided:

Embodiment 1 provides a compound of formula I-1:

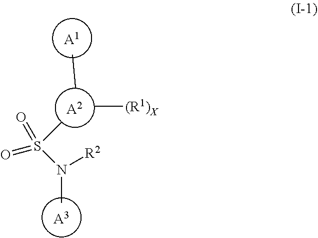

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteratoms independently selected from oxygen, nitrogen, and sulfur, wherein the heteroaryl and heterocyclyl are substituted with n occurrences of R⁶;
A² is pyrazolylene or 1,2,3-triazolylene;
A³ is

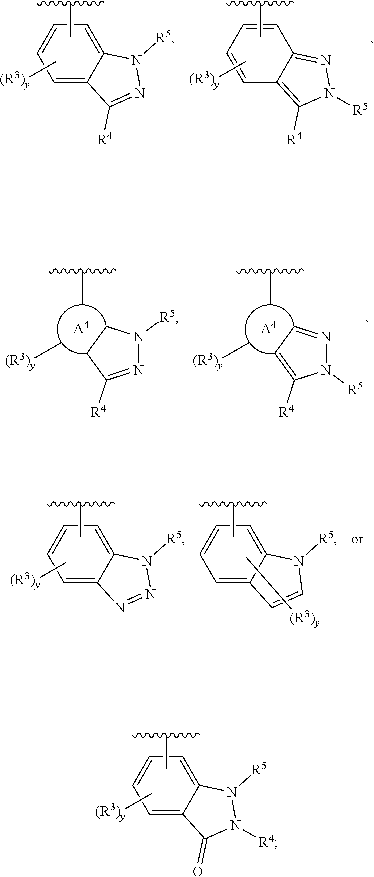

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R¹⁰)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R′)S(O₂)R¹⁰;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

Embodiment 2 provides the compound of embodiment 1, wherein the compound is a compound of Formula I-1.

Embodiment 3 provides the compound of embodiment 1 or 2, wherein A² is pyrazolylene.

Embodiment 4 provides the compound of embodiment 1 or 2, wherein A² is 1,2,3-triazolylene.

Embodiment 5 provides the compound of embodiment 1, wherein the compound is a compound of Formula Ia-1 or a pharmaceutically acceptable salt thereof:

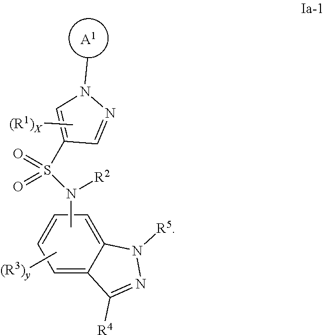

Embodiment 6 provides the compound of embodiment 1, wherein the compound is a compound of Formula Ib-1 or Ic-1 or a pharmaceutically acceptable salt thereof:

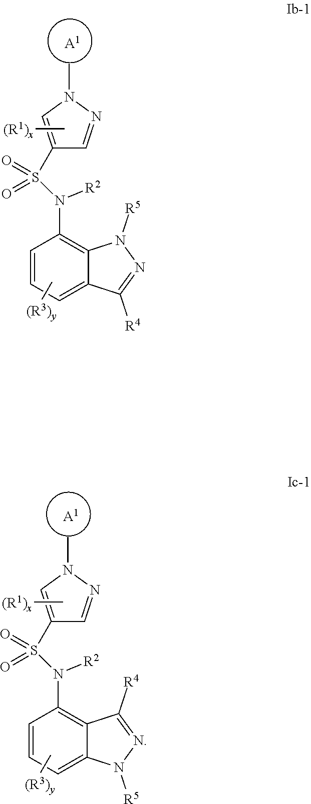

Embodiment 7 provides the compound of any one of embodiments 1-6, wherein y is 1.

Embodiment 8 provides the compound of embodiment 1, wherein the compound is a compound of Formula Id-1 or Ie-1 or a pharmaceutically acceptable salt thereof:

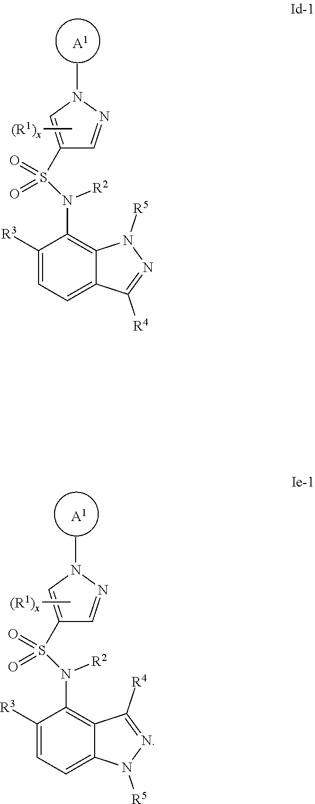

Embodiment 9 provides the compound of embodiment 1, wherein the compound is a compound of Formula If-1 or a pharmaceutically acceptable salt thereof:

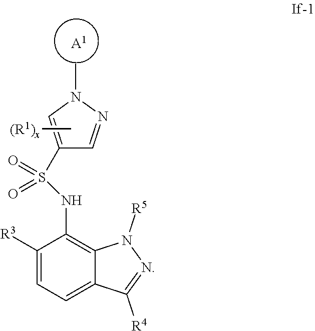

Embodiment 10 provides the compound ofany one ofembodiments 1-9, wherein x is 0.

Embodiment 11 provides the compound of embodiment 1, wherein the compound is a compound of Formula Ig-1 or Ih-1 a pharmaceutically acceptable salt thereof:

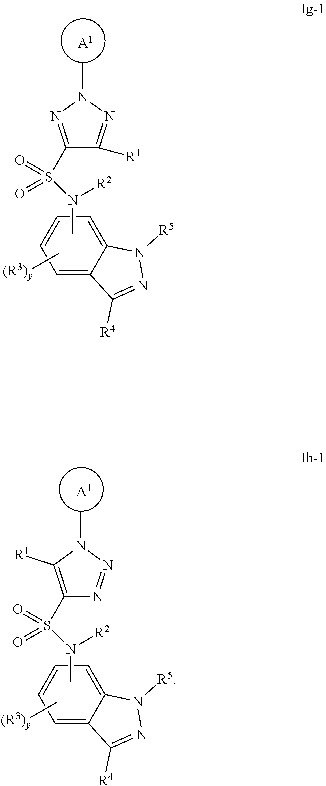

Embodiment 12 provides the compound of embodiment 1, wherein the compound is a compound of Formula II-1, Ij-1, Ik-1, or Il-1 or a pharmaceutically acceptable salt thereof:

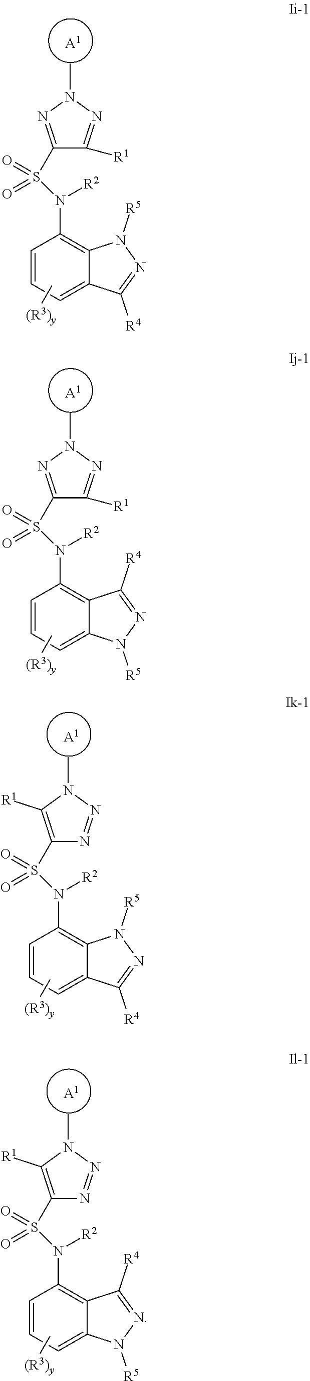

Embodiment 13 provides the compound of embodiment 11 or 12, wherein y is 1.

Embodiment 14 provides the compound of embodiment 1, wherein the compound is a compound of Formula Im-1, In-1, Io-1, or Ip-1 or a pharmaceutically acceptable salt thereof:

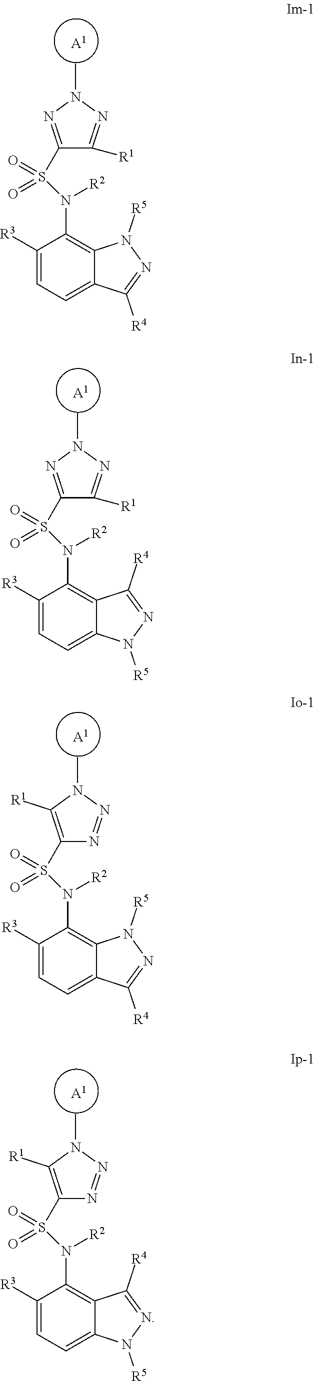

Embodiment 15 provides the compound of any one of embodiments 1-8 or 10-14, wherein R² is hydrogen.

Embodiment 16 provides the compound of any one of embodiments 1-15, wherein A¹ is a 6-membered heteroaryl containing 1, 2, or 3 heteratoms independently selected from oxygen, nitrogen, and sulfur, wherein the heteroaryl is substituted with n occurrences of R⁶.

Embodiment 17 provides the compound of any one of embodiments 1-15, wherein A¹ is pyridinyl, pyrimidinyl, pyridazinyl, or pyrazinyl, each of which is substituted with n occurrences of R⁶.

Embodiment 18 provides the compound of any one of embodiments 1-15, wherein A¹ is pyridinyl substituted with n occurrences of R⁶.

Embodiment 19 provides the compound of any one of embodiments 1-15, wherein A¹ is

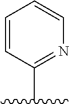 [SMILES: *c1ccccn1]

substituted with n occurrences of R⁶.

Embodiment 20 provides the compound of any one of embodiments 1-19, wherein n is 1.

Embodiment 21 provides the compound of any one of embodiments 1-19, wherein n is 2.

Embodiment 22 provides the compound of any one of embodiments 1-15, wherein A¹ is

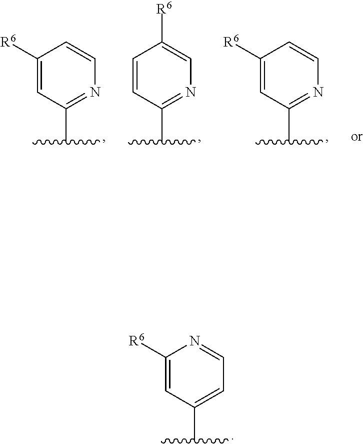

Embodiment 23 provides the compound of any one of embodiments 1-15, wherein A¹ is

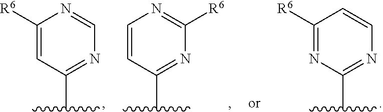

Embodiment 24 provides the compound of any one of embodiments 1-23, wherein R⁶ is C₁₋₆ haloalkyl.

Embodiment 25 provides the compound of any one of embodiments 1-23, wherein R⁶ is —CF₃.

Embodiment 26 provides the compound of any one of embodiments 1-23, wherein R⁶ is C₁₋₆ alkyl.

Embodiment 27 provides the compound of any one of embodiments 1-23, wherein R⁶ is methyl.

Embodiment 28 provides the compound of any one of embodiments 1-23, wherein R⁶ is C₃₋₇ cycloalkyl.

Embodiment 29 provides the compound of any one of embodiments 1-23, wherein R⁶ is cyclopropyl.

Embodiment 30 provides the compound of any one of embodiments 1-23, wherein R⁶ is halo.

Embodiment 31 provides the compound of any one of embodiments 1-30, wherein R⁵ is C₁₋₄ alkyl.

Embodiment 32 provides the compound of any one of embodiments 1-30, wherein R⁵ is methyl.

Embodiment 33 provides the compound of any one of embodiments 1-32, wherein R⁴ is hydrogen.

Embodiment 34 provides the compound of any one of embodiments 1-33, wherein R³ is C₁₋₆ alkyl.

Embodiment 35 provides the compound of any one of embodiments 1-33, wherein R³ is ethyl.

Embodiment 36 provides the compound of any one of embodiments 1-33, wherein R³ is C₃₋₇ cycloalkyl.

Embodiment 37 provides the compound of any one of embodiments 1-33, wherein R³ is cyclopropyl.

Embodiment 38 provides the compound of any one of embodiments 1-33, wherein R³ is C₁₋₆ alkoxyl.

Embodiment 39 provides the compound of any one of embodiments 1-33, wherein R³ is methoxy.

Embodiment 40 provides a compound represented by Formula II:

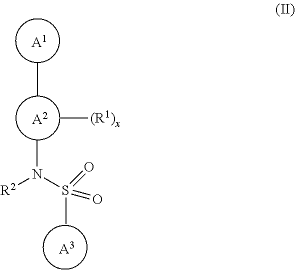

or a pharmaceutically acceptable salt thereof, wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteratoms independently selected from oxygen, nitrogen, and sulfur, wherein the heteroaryl and heterocyclyl are substituted with n occurrences of R⁶;
A² is pyrazolylene or 1,2,3-triazolylene;
A³ is

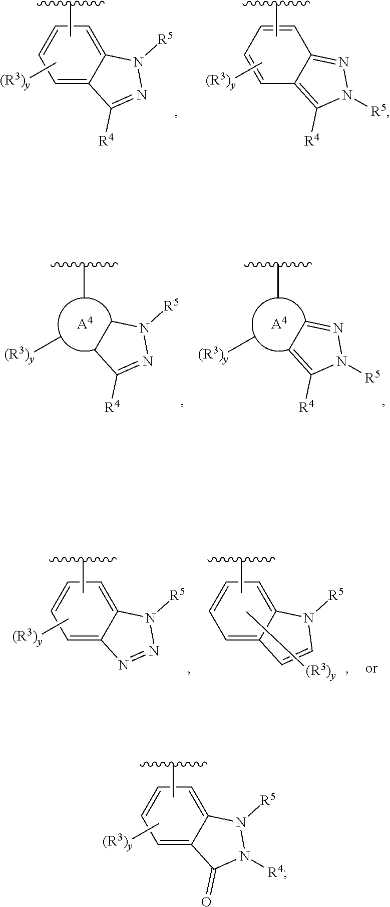

A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R¹⁰)C(O)R¹⁰, or —S(O₂)R¹⁰;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteratoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

Embodiment 41 provides the compound of embodiment 40, wherein the compound is a compound of Formula II.

Embodiment 42 provides the compound of embodiment 40 or 41, wherein A² is pyrazolylene.

Embodiment 43 provides the compound of any one of embodiments 40-42, wherein R² is hydrogen.

Embodiment 44 provides the compound of any one of embodiments 40-43, wherein A¹ is a 6-membered heteroaryl containing 1, 2, or 3 heteratoms independently selected from oxygen, nitrogen, and sulfur, wherein the heteroaryl is substituted with n occurrences of R⁶.

Embodiment 45 provides the compound of any one of embodiments 40-44, wherein A¹ is pyridinyl, pyrimidinyl, pyridazinyl, or pyrazinyl, each of which is substituted with n occurrences of R⁶.

Embodiment 46 provides the compound of any one of embodiments 40-45, wherein A¹ is pyridinyl substituted with n occurrences of R⁶.

Embodiment 47 provides the compound of any one of embodiments 40-46, wherein R⁶ is C₁₋₆ haloalkyl.

Embodiment 48 provides the compound of any one of embodiments 40-46, wherein R⁶ is —CF₃.

Embodiment 49 provides the compound of any one of embodiments 40-46, wherein R⁶ is C₁₋₆ alkyl.

Embodiment 50 provides the compound of any one of embodiments 40-49, wherein R³ is C₁₋₆ alkyl.

Embodiment 51 provides the compound of any one of embodiments 40-49, wherein R³ is C₃₋₇ cycloalkyl.

Embodiment 52 provides the compound of any one of embodiments 40-49, wherein R³ is C₁₋₆ alkoxyl.

Embodiment 53 provides a compound in Table 1 or 2, or a pharmaceutically acceptable salt thereof.

Embodiment 54 provides a pharmaceutical composition comprising a compound of any one of embodiments 1-53 and a pharmaceutically acceptable carrier.

Embodiment 55 provides a method for treating a disease or condition mediated by MALT1, comprising administering to a subject in need thereof a therapeutically effective amount of a compound of any one of embodiments 1-53 to treat the disease or condition.

Embodiment 56 provides the method of embodiment 55, wherein said disease or condition mediated by MALT1 is a proliferative disorder.

Embodiment 57 provides the method of embodiment 55, wherein said disease or condition mediated by MALT1 is an inflammatory disorder.

Embodiment 58 provides the method of embodiment 55, wherein said disease or condition mediated by MALT1 is an autoimmune disorder.

Embodiment 59 provides the method of embodiment 55, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

Embodiment 60 provides the method of embodiment 55, wherein said disease or condition mediated by MALT1 is cancer.

Embodiment 61 provides the method of embodiment 60, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

Embodiment 62 provides the method of embodiment 55, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

Embodiment 63 provides the method of embodiment 55, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythernatosus, systemic lupus erythematosus, Hashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

Embodiment 64 provides the method of embodiment 55, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

Embodiment 65 provides the method of any one of embodiments 55-65, wherein the subject is a human.

Embodiment 66 provides a method of inhibiting the activity of MALT1, comprising contacting a MALT1 with an effective amount of a compound of any one of embodiments 1-53 to inhibit the activity of said MALT1.

### EXAMPLES

The invention now being generally described, will be more readily understood by reference to the following examples, which are included merely for purposes of illustrating certain aspects and embodiments of the present invention, and are not intended to limit the invention.

The following general procedures were used in certain instances. Examples below may refer to one of the following general procedures. NMR chemical shift data are presented in ppm values.

### General Procedure A: For Coupling a Sulfonyl Chloride and Amine

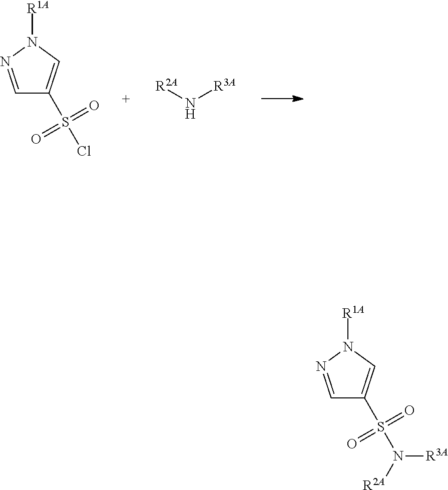

To a solution of amine (1.0 eq.) in pyridine are added the sulfonyl chloride compound (1.2-2.0 eq.) and 4-(dimethylamino)pyridine (0.1 eq.) at rt. The resulting mixture is heated at 50° C. for 20 h. Then, the reaction mixture solvent is evaporated under reduced pressure and the resulting residue is partitioned between ethyl acetate (EtOAc) and water. The organic phase from the mixture is isolated and dried over magnesium sulfate, filtered and concentrated in vacuum. The resulting residue is purified by preparative HPLC.

### General Procedure B: For Coupling a Sulfonyl Imidazole Salt and an Aminoindazole

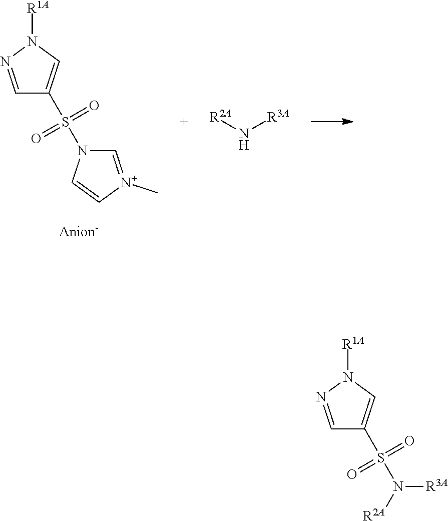

To a stirred solution of amine (1.0 eq.) in MeCN is added a sulfonyl imidazolium (1.0 eq.). The resulting mixture is stirred for 3 h at 90° C. Then, the reaction mixture solvent is evaporated under reduced pressure and the resulting residue is partitioned between EtOAc and water. The organic phase from the mixture is isolated and dried over magnesium sulfate, filtered and concentrated in vacuum. The resulting residue is purified by preparative HPLC.

### General Procedure C: For Coupling a Pyrazole Sulfonamide and Chloro-Heteroaryl

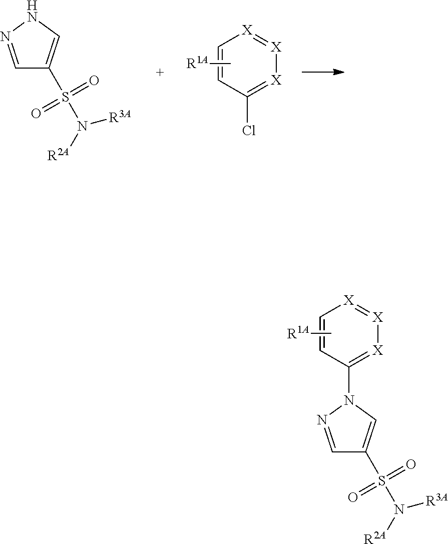

To a solution of 1H-Pyrazole-4-sulfonamide (1.0 eq) and chloropyridine (1.5 eq.) in DMF is added Cs₂CO₃ (3 eq.) at rt under nitrogen and then the resulting mixture is stirred at 100° C. for 16 h. Then, the resulting mixture is poured into water and extracted with EtOAc, the combined organic layers are washed with brine, dried over anhydrous Na₂SO₄, and concentrated under reduced pressure. The crude product is purified by preparative HPLC.

### General Procedure D: For Coupling a Pyrazole and a Bromo-Heteroaryl

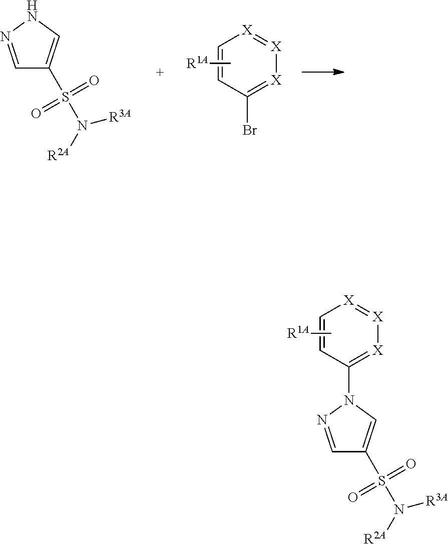

To a solution of 1H-Pyrazole-4-sulfonamide (1.0 eq.) and bromopyridine (1.5 eq.) in DMA is added CuI (0.2 eq.), Cs₂CO₃ (3 eq.) at rt under nitrogen and the mixture is stirred at 120° C. for 16 h. The resulting mixture is then poured into water and extracted with EtOAc, the combined organic layers are washed with brine, dried over anhydrous Na₂SO₄, and concentrated under reduced pressure. The crude product is purified by preparative HPLC.

### General Procedure E: For Installing a Methyl Group on the Sulfonamide Nitrogen

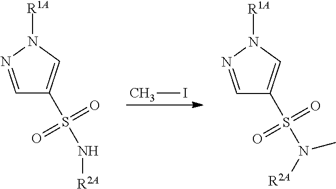

A solution of sulfonamide (1 eq.) in DMF is treated with Cs₂CO₃ (3 eq.) and Mel (1.5 eq.) at rt for 4 h. Then, the resulting mixture is quenched with H₂O and extracted with EtOAc. The combined organic layers from the mixture are washed with brine, dried over anhydrous Na₂SO₄, and concentrated under reduced pressure. The resulting residue is purified by preperative HPLC.

### General Procedure F: For Synthesis of a Sulfonyl Imidazolium Intermediate Compound

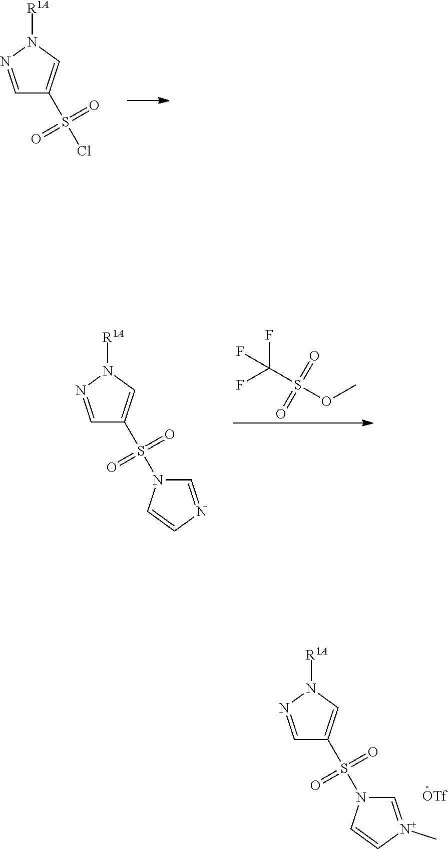

Imidazole (3 eq.) is suspended in CH₂Cl₂ (DCM) and cooled to 0° C. in an ice bath. A DCM solution of an aryl sulfonyl chloride (1 eq.) is added dropwise over 15 min. Then, the reaction mixture is stirred at rt for 3 h, and then filtered. The solid is washed with DCM, and the filtrate is washed with brine. The organic layer is dried over Na₂SO₄, and concentrated under reduced pressure. The crude sulfonyl imidazole product can be purified through recrystallization. Methyl trifluoromethanesulfonate (5 eq) is added dropwise to a solution of sulfonyl imidazole (1 eq.) in DCM over 15 min at 0° C. The reaction mixture is stirred at rt for 1 h at 0° C., and then filtered. The resulting solid is washed with EtOAc, and the filtrate is washed with brine. The organic layer is dried over Na₂SO₄, and concentrated under reduced pressure.

### Example 1—Synthesis of 6-Cyclopropyl-1-Methyl-7-Nitroindazole

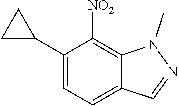 [SMILES: Cn1ncc2ccc(C3CC3)c([N+](=O)[O-])c21]

To a stirred mixture of 6-chloro-1-methyl-7-nitroindazole (5.0 g, 24 mmol, 1.0 eq.) and cyclopropylboronic acid (6.1 g, 71 mmol, 3.0 eq.) in 1,4-dioxane (30 mL) and H₂O (4 mL) was added K₂CO₃ (6.5 g, 47 mmol, 2.0 eq.) and Pd(dppf)Cl₂ (0.86 g, 1.2 mmol, 0.05 eq.) in portions at 100° C. under nitrogen. The mixture was allowed to cool to rt, and then extracted with EtOAc (4×50 mL). The combined organic layers were dried over anhydrous Na₂SO₄, and evaporated under reduced pressure. The residue was purified by silica gel column chromatography, eluted with petroleum ether/ethyl acetate (PE/EtOAc) (1:1 vol/vol) to afford the title compound (3.5 g, 41%) as a brown solid. (ES, m/z): [M+H]+ 218; ¹H NMR (400 MHz, CDCl₃) δ 0.70-0.80 (m, 2H), 1.00-1.2 (m, 2H), 2.10-2.25 (m, 1H), 4.00 (s, 3H), 6.82 (d, J=8.4 Hz, 1H), 7.74 (d, J=8.4 Hz, 1H), 8.01 (s, 1H).

### Example 2—Synthesis of 6-Cyclopropyl-1-Methyl-Indazol-7-Amine

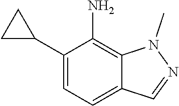

A mixture of 6-cyclopropyl-1-methyl-7-nitroindazole (3.5 g, 9 mmol, 1.0 eq.) and Pd/C (2 g, 19 mmol, 2.0 eq.) in EtOAc was stirred for 3 h at rt under hydrogen. The resulting mixture was filtered, and the solid was washed with EtOAc (2×200 mL). The filtrate was concentrated under reduced pressure, and the resulting residue was purified by reversed-phase flash chromatography with the following conditions: column: C18 silica gel; mobile phase: MeCN in water (0.1% TFA), 20% to 50% gradient in 20 min; detector: UV 254 nm. The resulting mixture was concentrated under reduced pressure. This yielded the title compound (2.7 g, 45%) as a brown solid. (ES, m/z): [M+H]⁺ 188; ¹H NMR (400 MHz, CDCl₃) δ 0.40-0.60 (m, 2H), 0.85-1.00 (m, 2H), 1.75-1.90 (m, 1H), 4.29 (s, 3H), 5.00 (s, 2H), 6.71 (d, J=8.4 Hz, 1H), 6.91 (d, J=8.4 Hz, 1H), 7.76 (s, 1H).

### Example 3—Synthesis of 7-Nitro-1H-Indazol-6-Yl Trifluoro-Methanesulfonate

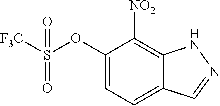 [SMILES: O=[N+]([O-])c1c(OS(=O)(=O)C(F)(F)F)ccc2cn[nH]c12]

To a stirred solution of 7-nitro-1H-indazol-6-ol (5 g, 28 mmol, 1.0 eq.) and TEA (7.8 mL, 56 mmol, 2.0 eq.) in DCM (30 mL) was added trifluoromethanesulfonyl chloride (4.5 mL, 42 mmol, 1.5 eq.) dropwise at 0° C. under nitrogen. The resulting mixture was stirred for 2 h at rt, and then diluted with water (30 mL). The resulting mixture was extracted with CH₂Cl₂ (3×30 mL). The combined organic layers were washed with brine (2×20 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min to afford the title compound (1.7 g, 19%) as a yellow solid. (ES, m/z): [M+H]⁺ 312.

### Example 4—Synthesis of 6-[2-(Tert-Butyldimethylsilyl)Ethynyl]-7-Nitro-1H-Indazole

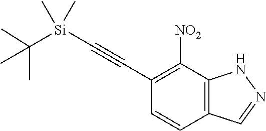 [SMILES: CC(C)(C)[Si](C)(C)C#Cc1ccc2cn[nH]c2c1[N+](=O)[O-]]

To a stirred solution of 7-nitro-1H-indazol-6-yl trifluoromethanesulfonate (1.5 g, 4.8 mmol, 1.0 eq.), Et₃N (3.4 mL, 24 mmol, 5.0 eq.) and CuI (184 mg, 1.0 mmol, 0.2 eq.) in MeCN (10 mL) were added Pd(PPh₃)₂Cl₂ (338 mg, 0.5 mmol, 0.1 eq.) and tert-butyl(ethynyl)dimethylsilane (4.5 mL, 24 mmol, 5.0 eq.). The resulting mixture was stirred 16 h at rt under nitrogen, and then was diluted with water (20 mL), and extracted with EtOAc (3×10 mL). The combined organic layers were washed with brine (2×30 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min; detector: UV 254 nm. This resulted in the title compound (700 mg, 43%) as a yellow solid. (ES, m/z): [M−H]⁺302.

### Example 5—Synthesis of 6-[2-(Tert-Butyldimethylsilyl)Ethyny]-1-methyl-7-Nitroindazole

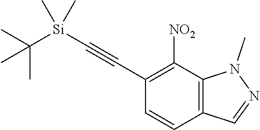 [SMILES: Cn1ncc2ccc(C#C[Si](C)(C)C(C)(C)C)c([N+](=O)[O-])c21]

To a stirred solution of 6-[2-(tert-butyldimethylsilyl)ethynyl]-7-nitro-1H-indazole (700 mg, 2.3 mmol, 1.0 eq.) and Cs₂CO₃ (1.5 g, 4.6 mmol, 2.0 eq.) in DMF (10 mL) was added CH₃I (0.29 mL, 4.6 mmol, 2.0 eq.). The resulting mixture was stirred for 2 h at rt under nitrogen. The resulting mixture was diluted with water (50 mL). The resulting mixture was extracted with EtOAc (3×20 mL). The combined organic layers were washed with brine (2×30 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min; detector: UV 254 nm to afford the title compound (280 mg, 34%) as a yellow solid. (ES, m/z): [M−H]+ 316; ¹H NMR (400 MHz, DMSO-d₆) δ 0.20 (s, 6H), 0.98 (s, 9H), 3.93 (s, 3H), 7.38 (d, J=8.4 Hz, 1H), 8.09 (d, J=8.4 Hz, 1H), 8.37 (s, 1H).

### Example 6—Synthesis of 6-[2-(Tert-Butyldimethylsilyl)Ethynyl]-1-methylindazol-7-Amine

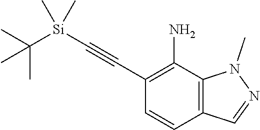 [SMILES: Cn1ncc2ccc(C#C[Si](C)(C)C(C)(C)C)c(N)c21]

To a stirred solution of 6-[2-(tert-butyldimethylsilyl)ethynyl]-1-methyl-7-nitroindazole (330 mg, 1.0 mmol, 1.0 eq.) and NH₄Cl (560 mg, 10.5 mmol, 10 eq.) in H₂O (5 mL) and EtOH (5 mL) was added Fe (584 mg, 10.5 mmol, 10 eq.). The resulting mixture was stirred for 6 h at rt under nitrogen, and then was filtered. The solid was washed with EtOAc (3×10 mL), and the filtrate was partitioned and extracted with EtOAc (10 mL). The combined organic layers were dried over anhydrous Na₂SO₄, and concentrated under vacuum to afford the title intermediate (260 mg, 70%) as a light yellow oil. (ES, m/z): [M+H]⁺286.

### Example 7—Synthesis of 6-Methoxy-1-Methyl-7-Nitroindazole

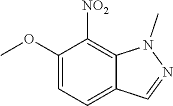

A solution of 6-chloro-1-methyl-7-nitroindazole (300 mg, 1.4 mmol, 1.0 eq.) and MeONa (766 mg, 14 mmol, 10 eq.) in MeOH (10 mL) was stirred for 3 h at 60° C. The resulting mixture was allowed to cool to rt, and diluted with water (50 mL). The resulting mixture was extracted with EtOAc (50 mL). The organic layer was washed with brine, dried over anhydrous Na₂SO₄, and concentrated under reduced pressure to afford the title compound (250 mg, 77%) as a yellow solid. (ES, m/z): [M+H]⁺ 208.

### Example 8—Synthesis of 6-Methoxy-1-Methylindazol-7-Amine

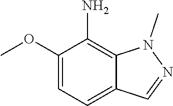

A solution of 6-methoxy-1-methyl-7-nitroindazole (230 mg, 1.1 mmol, 1.0 eq.) and Pd/C (130 mg, 1.2 mmol, 1.1 eq.) in MeOH (5 mL) was stirred for 2 h at rt under hydrogen. The resulting mixture was then filtered and the solid was washed with MeOH (3×10 mL). The filtrate was concentrated under reduced pressure to afford the title compound (195 mg, 89%) as an off-white oil.(ES, m/z): [M+H]+ 178.

### Example 9—Synthesis of 6-Ethoxy-1-Methyl-7-Nitroindazole

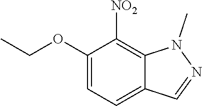 [SMILES: CCOc1ccc2cnn(C)c2c1[N+](=O)[O-]]

6-Chloro-1-methyl-7-nitroindazole (400 mg, 1.9 mmol, 1.0 eq.) was treated with sodium ethanolate (1.29 g, 19 mmol, 10 eq.) in EtOH (5 mL) in a similar procedure as that reported for 6-Methoxy-1-Methyl-7-nitroindazole to afford the title compound (465 mg, 63%) as a white solid. (ES, m/z): [M+H]+ 222.

### Example 10—Synthesis of 6-Ethoxy-1-Methylindazol-7-Amine

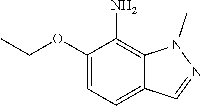

The title compound was obtained through the same Fe/NH₄Cl reduction as reported for 6-methoxy-1-methylindazol-7-amine using 6-ethoxy-1-methyl-7-nitroindazole instead. A white solid was obtained (165 mg, 41%). (ES, m/z): [M+H]⁺ 192.

### Example 11—Synthesis of 6-Isopropoxy-1-Methyl-7-Nitro-1H-Indazole

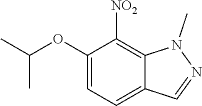 [SMILES: CC(C)Oc1ccc2cnn(C)c2c1[N+](=O)[O-]]

6-Chloro-1-methyl-7-nitroindazole (400 mg, 1.9 mmol, 1.0 eq.) was treated with sodium propan-2-olate (1.6 g, 19 mmol, 10 eq.) in isopropyl alcohol (20 mL) in a similar procedure as that reported for 6-Methoxy-1-Methyl-7-nitroindazole to afford the title compound (410 mg, 92%) as a white solid. (ES, m/z): [M+H]⁺ 236.

### Example 12—Synthesis of 6-Isopropoxy-1-Methyl-1H-Indazol-7-Amine

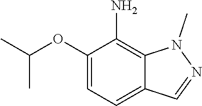

A mixture of 6-isopropoxy-1-methyl-7-nitroindazole (200 mg, 0.85 mmol, 1.0 eq.) and Pd/C (181 mg, 1.7 mmol, 2.0 eq.) in MeOH (10 mL) was stirred for 2 h at rt under hydrogen. The reaction was quenched by the addition of MeOH (10 mL) at rt. The resulting mixture was filtered and the solid was washed with MeOH (3×10 mL). The filtrate was concentrated under reduced pressure to afford the title compound (142 mg, 81%) as a brown solid. (ES, m/z): [M+H]⁺ 206.

### Example 13—Synthesis of 2-(7-amino-1-Methyl-1H-Indazol-6-yl)Acetonitrile

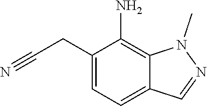

The title compound was obtained through the same Fe/NH₄Cl reduction as reported for 6-methoxy-1-methylindazol-7-amine using 2-(1-methyl-7-nitroindazol-6-yl)acetonitrile instead. A red-brown solid was obtained (120 mg, 59%). (ES, m/z): [M+H]⁺ 187.

### Example 14—Synthesis of N,N,1-Trimethyl-7-Nitroindazol-6-Amine

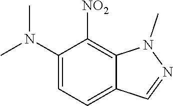 [SMILES: CN(C)c1ccc2cnn(C)c2c1[N+](=O)[O-]]

To a stirred solution of 6-chloro-1-methyl-7-nitroindazole (1 g, 4.7 mmol, 1.0 eq.) in dimethylamine (2 M in DMF, 10 mL) was added K₂CO₃ (1.3 g, 9.5 mmol, 2.0 eq.). The resulting mixture was stirred for 2 h at 150° C. under nitrogen. The resulting mixture was cooled to rt and diluted with water (50 mL). The resulting mixture was extracted with EtOAc (3×20 mL), the combined organic layers were washed with brine (2×30 mL), dried over anhydrous Na₂SO₄, and concentrated under reduced pressure. The residue was purified by reversed phase flash chromatography with the following conditions: column: C18 silica gel; mobile phase: MeCN in water (0.1% TFA), 20% to 40% gradient in 10 min; detector: UV 254 nm. This resulted in the title compound (500 mg, 48%) as a yellow oil. (ES, m/z): [M+H]⁺ 221; ¹H NMR (400 MHz, DMSO-d₆) δ 2.87 (s, 6H), 3.85 (s, 3H), 7.13 (d, J=8.8 Hz, 1H), 7.86 (d, J=8.8 Hz, 1H), 8.10 (s, 1H).

### Example 15—Synthesis of N6,N6,1-Trimethylindazole-6,7-Diamine

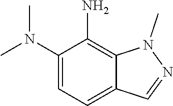

To a stirred solution of N,N,1-trimethyl-7-nitroindazol-6-amine (450 mg, 2.0 mmol, 1.0 eq.) in EtOAc (10 mL) was added Pd/C (43 mg, 0.41 mmol, 0.2 eq.) at rt under hydrogen. The resulting mixture was filtered, the solid washed with EtOAc (3×100 mL), and the filtrate was concentrated under reduced pressure. The resulting residue was purified by reversed phase flash chromatography with the following conditions: column: C18 silica gel; mobile phase: MeCN in water (0.1% TFA), 10% to 30% gradient in 10 min; detector: UV 254 nm to afford the title compound (300 mg, 77%) as a purple oil. (ES, m/z): [M+H]⁺ 191; ¹H NMR (400 MHz, DMSO-d₆) δ 2.60 (s, 6H), 4.26 (s, 3H), 4.90 (s, 2H), 6.96 (d, J=8.4 Hz, 1H), 7.01 (d, J=8.4 Hz, 1H), 7.78 (s, 1H).

### Example 16—Synthesis of 1-Methyl-6-Propyl-1H-Indazol-7-Amine

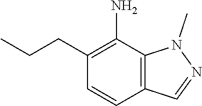

A mixture of 6-cyclopropyl-1-methyl-7-nitroindazole (4 g, 18 mmol, 1 eq.) and Pd/C (2.0 g, 18 mmol, 1 eq.) in EtOAc was stirred for 3 h at rt under hydrogen. The resulting mixture was filtered, the solid was washed with EtOAc (2×100 mL), and the filtrate was concentrated under reduced pressure. The residue was purified by reversed-phase flash chromatography with the following conditions: column: C18 silica gel; mobile phase: MeCN in water (0.1% FA), 20% to 50% gradient in 20 min; detector: UV 254 nm. The resulting mixture was concentrated under reduced pressure. The title compound was obtained as a by-product (150 mg, 4%). (ES, m/z): [M+H]⁺ 190; ¹H NMR (400 MHz, DMSO-d₆) δ 0.94 (t, J=7.6 Hz, 3H), 1.50-1.60 (m, 2H), 2.53-2.63 (m, 2H), 4.29 (s, 3H), 6.75 (d, J=8.0 Hz, 1H), 6.92 (d, J=8.0 Hz, 1H), 7.75 (s, 1H).

### Example 17—Synthesis of 5-Bromo-3-Iodo-4-Nitro-1H-Indazole

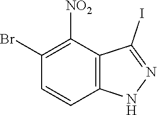 [SMILES: O=[N+]([O-])c1c(Br)ccc2[nH]nc(I)c12]

A mixture of 5-bromo-4-nitro-1H-indazole (4.0 g, 17 mmol, 1 eq.) and NIS (7.4 g, 33 mmol, 2 eq.) in DMF (50 mL) was stirred for 2 h at rt under nitrogen. The resulting mixture was diluted with water (200 mL) and extracted with EtOAc (3×50 mL). The combined organic layers were washed with brine (200 mL), dried over anhydrous Na₂SO₄, and concentrated under reduced pressure. The residue was purified by silica gel column chromatography, and eluted with (PE:EA=30:1) to afford the title compound (2.5 g, 33%) as a yellow solid. (ES, m/z): [M+H]+ 368/370.

### Example 18—Synthesis of 5-Bromo-3-Methyl-4-Nitro-1H-Indazole

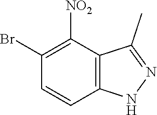

To a stirred solution of 5-bromo-3-iodo-4-nitro-1H-indazole (500 mg, 1.4 mmol, 1 eq.) and Pd(dppf)Cl₂ (99 mg, 0.14 mmol, 0.1 eq.) in dioxane (10 mL) was added dimethylzinc (1.2 mL, 1.2 mmol, 0.9 eq.) dropwise at rt under nitrogen, and the resulting mixture was stirred for 5 h at 60° C. The mixture was acidified with 2 M HCl, and then diluted with water (20 mL) and extracted with EtOAc (3×10 mL). The combined organic layers were washed with brine (50 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm to afford the title compound (50 mg, 14%) as a yellow solid. (ES, m/z): [M+H]⁺ 256/258.

### Example 19—Synthesis of 3-Methyl-4-Nitro-5-Vinyl-1H-Indazole

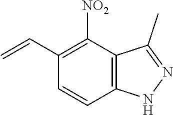

A solution of 5-bromo-3-methyl-4-nitro-1H-indazole (1.9 g, 7.4 mmol, 1 eq.) in the mixture of 1,4-dioxane (16 mL) and H₂O (4 mL), was treated with potassium vinyltrifluoroborate (1.1 g, 8.2 mmol, 1.1 eq.), Pd(dppf)Cl₂ (0.5 g, 0.7 mmol, 0.1 eq.) and TEA (2.6 mL, 19 mmol, 2.5 eq.). The resulting mixture was stirred for an additional 2 h at 100° C. The resulting mixture was then diluted with water (50 mL) and extracted with EtOAc (3×30 mL). The combined organic layers were washed with water (2×50 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm. This resulted in the title compound (630 mg, 42%) as a yellow solid. (ES, m/z): [M+H]⁺ 204.

### Example 20—Synthesis of 5-Ethyl-3-Methyl-1H-Indazol-4-Amine

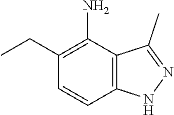

To a stirred solution 5-ethenyl-3-methyl-4-nitro-1H-indazole (300 mg, 1.5 mmol, 1 eq.) in MeOH (10 mL) was added Pd/C (30 mg, 0.3 mmol, 0.2 eq.) at rt under nitrogen. The resulting mixture was stirred for 2 h at rt under hydrogen, and then filtered. The solids were washed with MeOH (3×10 mL), and filtrate was concentrated under reduced pressure. The residue was purified by reverse flash chromatography with the following conditions: column: C18 silica gel; mobile phase: MeCN in water (0.1% TFA), 0% to 100% gradient in 40 min; detector: UV 254 nm to afford the title compound (200 mg, 77%) as a yellow oil. (ES, m/z): [M+H]⁺ 176.

### Example 21—Synthesis of 1,3-Dimethyl-4-Nitro-5-Vinyl-1H-Indazole

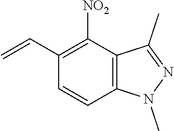 [SMILES: C=Cc1ccc2c(c(C)nn2C)c1[N+](=O)[O-]]

To a stirred mixture of 5-ethenyl-3-methyl-4-nitro-1H-indazole (310 mg, 1.5 mmol, 1 eq.) and Cs₂CO₃ (994 mg, 3 mmol, 2 eq.) in DMF (3 mL) was added CH₃I (0.19 mL, 3 mmol, 2 eq.). The resulting mixture was stirred for an additional 2 h at rt and then was diluted with water (10 mL) and extracted with EtOAc (3×5 mL). The combined organic layers were washed with water (2×10 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm to afford the title compound (170 mg, 51%) as a yellow oil. (ES, m/z): [M+H]⁺ 218.

### Example 22—Synthesis of 5-Ethyl-1,3-Dimethyl-1H-Indazol-4-Amine

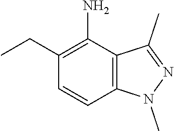

A mixture of 5-ethenyl-1,3-dimethyl-4-nitroindazole (200 mg, 0.9 mmol, 1 eq.) and Pd/C (98 mg, 0.9 mmol, 1 eq.) in MeOH (5 mL) was stirred for 2 h at rt under hydrogen. The resulting mixture was filtered and the solids were washed with MeOH (3×10 mL). The filtrate was concentrated under reduced pressure, and the residue was purified by reverse flash chromatography with the following conditions: column: C18 silica gel; mobile phase: MeCN in water (0.1% TFA), 0% to 100% gradient in 30 min; detector: UV 254 nm to afford the title compound (180 mg, 88%) as a yellow oil. (ES, m/z): [M+H]⁺ 190.

### Example 23—Synthesis of 1-Methyl-7-Nitro-5-(4,4,5,5-Tetramethyl-1,3,2-Dioxaborolan-2-yl)-1H-Indazole

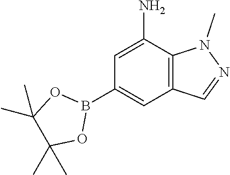 [SMILES: Cn1ncc2cc(B3OC(C)(C)C(C)(C)O3)cc(N)c21]

To a stirred solution of 5-bromo-1-methyl-7-nitro-1H-indazole (847 mg, 3.7 mmol, 1 eq.) in DMSO (40 mL) were added KOAc (0.65 g, 6.6 mmol, 2 eq.), Pd(dppf)Cl₂ (0.24 g, 0.3 mmol, 0.1 eq.) and bis(pinacolato)diboron (1.68 g, 6.6 mmol, 2 eq.) in portions at rt under nitrogen and the resulting mixture was stirred for 16 h at 80° C. The mixture was then extracted with EtOAc (3×40 mL), the combined organic layers were washed with brine (40 mL), dried over anhydrous Na₂SO₄, and concentrated under reduced pressure. The title compound (1.9 g) was used in the next step directly without further purification. (ES, m/z): [M+H]⁺ 304; ¹H NMR (400 MHz, DMSO-d₆) δ 1.34 (s, 12H), 4.16 (s, 3H), 8.26 (s, 1H), 8.42 (s, 1H), 8.52 (s, 1H).

### Example 24—Synthesis of 1-Methyl-7-Nitro-1H-indazol-5-ol

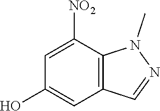

A solution of 1-methyl-7-nitro-5-(4,4,5,5-tetramethyl-1,3,2-dioxaborolan-2-yl)-1H-indazole (1.9 g, 6.3 mmol, 1 eq.) and NH₄HCO₃ (0.51 g, 6.5 mmol, 1 eq.) in acetonitrile (20 mL) was treated with hydrogen peroxide (0.44 g, 13 mmol, 2 eq.) for 2 h at rt under nitrogen. The resulting mixture was concentrated under reduced pressure and then disolved with water (20 mL), and extracted with EtOAc (3×20 mL). The combined organic layers were washed with brine (30 mL), dried over anhydrous Na₂SO₄, and concentrated under reduced pressure. The resulting residue was then purified by purified by reversed-phase flash chromatography with the following conditions: column: C18 silica gel; mobile phase: MeCN in Water (10 mmol/L NH₄HCO₃), 40% to 50% gradient in 30 min; detector: UV 220 nm to afford the title compound (550 mg, 44%) as a yellow solid. (ES, m/z): [M+H]⁺ 194; ¹H NMR (400 MHz, DMSO-d₆) δ 4.08 (s, 3H), 7.50 (d, J=2.0 Hz, 1H), 7.67 (d, J=2.0 Hz, 1H), 8.16 (s, 1H), 10.00 (s, 1H).

### Example 25—Synthesis of 5-Methoxy-1-Methyl-7-Nitro-1H-Indazole

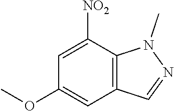

To a solution of 1-methyl-7-nitro-1H-indazol-5-ol (250 mg, 1.3 mmol, 1 eq.) in MeCN (5 mL) was added Cs₂CO₃ (843 mg, 2.6 mmol, 2 eq.) and the mixture was stirred for 5 min at rt under nitrogen. Then Mel (220.5 mg, 1.553 mmol, 1.2 eq.) was added dropwise at rt, and the resulting mixture was stirred for 2 h at rt under nitrogen. The mixture was the filtered, the solids were washed with MeCN (3×5 mL), and the filtrate was concentrated under reduced pressure to afford in the title compound (190 mg, 71%) as a yellow solid. (ES, m/z): [M+H]⁺ 208; ¹H NMR (400 MHz, CDCl₃) δ 3.90 (s, 3H), 4.21 (s, 3H), 7.41 (d, J=2.4 Hz, 1H), 7.78 (d, J=2.4 Hz, 1H), 8.02 (s, 1H).

### Example 26—Synthesis of 5-Methoxy-1-Methyl-1H-Indazol-7-Amine

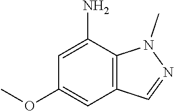

A solution of 5-methoxy-1-methyl-7-nitro-1H-indazole (200 mg, 1.0 mmol, 1 eq.) and Pd/C (21 mg, 0.2 mmol, 0.2 eq.) in EtOAc (12 mL) was stirred for 1 h at rt under hydrogen. The resulting mixture was filtered, the solids were washed with EtOAc (3×10 mL), and the filtrate was concentrated under reduced pressure. The crude product (200 mg, brown solid) was used in the next step directly without further purification. (ES, m/z): [M+H]⁺ 178; ¹H NMR (400 MHz, CDCl₃) δ 3.80 (s, 3H), 4.33 (s, 3H), 6.37 (d, J=2.0 Hz, 1H), 6.56 (d, J=2.4 Hz, 1H), 7.76 (s, 1H).

### Example 27—Synthesis of N-(6-Ethyl-1-Methyl-1H-Indazol-7-yl)-1-Trityl-1H-Pyrazole-4-Sulfonamide

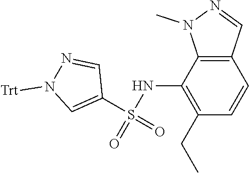

A solution of 1-trityl-1H-Pyrazole-4-sulfonyl chloride (4 g, 9.8 mmol, 1 eq.) and 6-ethyl-1-methylindazol-7-amine (2.1 g, 11.7 mmol, 1.2 eq.) in pyridine (40 mL) was stirred for 2 h at 80° C. under nitrogen. The resulting mixture was extracted with EtOAc (2×100 mL). The combined organic layers were washed with brine (200 mL), dried over anhydrous Na₂SO₄, and concentrated under reduced pressure. The residue was purified by reverse flash chromatography with the following conditions: column: C18 silica gel; mobile phase: MeCN in water (0.1% TFA), 10% to 50% gradient in 10 min; detector: UV 254 nm to afford the title compound (2.3 g, 43%) as a light brown solid. (ES, m/z): [M−1]⁻ 546; ¹H NMR (400 MHz, DMSO-d₆) δ 0.97 (t, J=7.5 Hz, 3H), 2.06 (d, J=57.8 Hz, 2H), 4.17 (s, 3H), 5.76 (s, 2H), 7.07-6.97 (m, 6H), 7.46-7.31 (m, 9H), 7.63 (d, J=8.3 Hz, 1H), 7.88 (d, J=0.7 Hz, 1H), 7.98 (s, 1H), 9.88 (s, 1H).

### Example 28—Synthesis of N-(6-Ethyl-1-Methyl-1H-Indazol-7-yl)-1H-Pyrazole-4-Sulfonamide

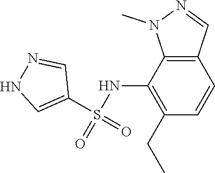 [SMILES: CCc1ccc2cnn(C)c2c1NS(=O)(=O)c1cn[nH]c1]

A solution N-(6-Ethyl-1-Methyl-1H-Indazol-7-yl)-1-trityl-1H pyrazole-4-sulfonamide (6 g, 11 mmol, 1 eq.) and TFA (16 mL, 219 mmol, 20 eq.) in DCM (50 mL) was stirred for 2 h at rt. The resulting mixture was extracted with EtOAc (2×100 mL), the combined organic layers were washed with brine (4×100 mL), dried over anhydrous Na₂SO₄, and concentrated under reduced pressure. The residue was purified by reverse flash chromatography with the following conditions: column: C18 silica gel; mobile phase: MeCN in water (0.1% TFA), 10% to 50% gradient in 10 min; detector: UV 254 nm to afford the title compound (2.0 g, 60%) as an off-white solid. (ES, m/z): [M+H]⁺ 306; ¹H NMR (400 MHz, DMSO-d₆) δ 0.88 (d, J=7.6 Hz, 3H), 2.19 (q, J=7.6 Hz, 2H), 4.22 (s, 4H), 6.98 (d, J=8.4 Hz, 1H), 7.62 (d, J=8.4 Hz, 2H), 7.98 (s, 1H), 8.12 (s, 1H), 9.75 (s, 1H), 13.59 (s, 1H).

### Example 29—Synthesis of 1-(2-(Trifluoromethyl)Pyridin-4-yl)-Pyrazole-4-Sulfonyl Chloride

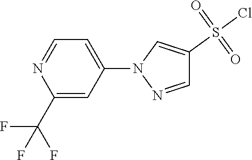 [SMILES: O=S(=O)(Cl)c1cnn(-c2ccnc(C(F)(F)F)c2)c1]

A solution of 4-(pyrazol-1-yl)-2-(trifluoromethyl)pyridine (13 g, 61 mmol, 1.0 eq.) and chlorosulfonic acid (100 mL) was stirred for 16 h at 100° C. The reaction was then quenched with HCl/ice, and the resulting mixture was extracted with DCM (2×120 mL). The combined organic layers were washed with brine (2×40 mL), dried over anhydrous Na₂SO₄, and concentrated under reduced pressure to afford the title sulfonyl chloride (19 g, 100%) as a brown yellow solid. (ES, m/z): [M+H]⁺ 312; ¹H NMR (400 MHz, DMSO-d₆) δ 7.92 (dd, J=5.6, 2.1 Hz, 1H), 8.15 (d, J=2.1 Hz, 1H), 8.78 (s, 1H), 8.28 (s, 1H), 8.93 (d, J=5.6 Hz, 1H).

### Example 30—Synthesis of 1-(5-(Trifluoromethyl)Pyridin-3-yl)-Pyrazole-4-Sulfonyl Chloride

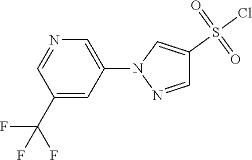 [SMILES: O=S(=O)(Cl)c1cnn(-c2cncc(C(F)(F)F)c2)c1]

3-(pyrazol-1-yl)-5-(trifluoromethyl)pyridine (90 mg, 0.42 mmol, 1.0 eq.) was treated with chlorosulfonic acid (8 mL) in a similar procedure as that reported for 1-(2-(Trifluoromethyl)Pyridin-4-yl)-pyrazole-4-sulfonyl chloride to afford the title intermediate (31 mg, 24%) as a yellow solid. (ES, m/z): [M+H]⁺ 312, ¹H NMR (400 MHz, DMSO-d₆) δ 7.81 (s, 1H), 8.66 (d, J=2.4 Hz, 1H), 8.93-8.84 (m, 2H), 9.44 (d, J=2.4 Hz, 1H).

### Example 31—Synthesis of 1-(4-(Trifluoromethyl)Pyridin-2-yl)-pyrazole-4-Sulfonyl Chloride

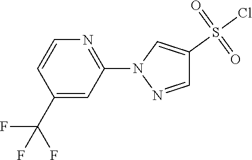 [SMILES: O=S(=O)(Cl)c1cnn(-c2cc(C(F)(F)F)ccn2)c1]

2-(pyrazol-1-yl)-4-(trifluoromethyl)pyridine (200 mg, 0.94 mmol, 1.0 eq.) was treated with chlorosulfonic acid (3 mL) in a similar procedure as that reported for 1-(2-(Trifluoromethyl)Pyridin-4-yl)-pyrazole-4-sulfonyl chloride to afford the title intermediate (200 mg) as a crude yellow oil. (ES, m/z): [M+H]⁺ 214.

### Example 32—Synthesis of 1-(6-(Trifluoromethyl)Pyrimidin-4-yl)-pyrazole-4-Sulfonyl Chloride

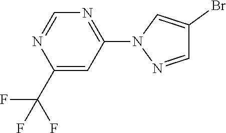 [SMILES: FC(F)(F)c1cc(-n2cc(Br)cn2)ncn1]

4-chloro-6-(trifluoromethyl)pyrimidine (500 mg, 2.7 mmol, 1.0 eq.) was added to a stirred suspension of 4-bromopyrazole (403 mg, 2.7 mmol, 1.0 eq.) and potassium carbonate (757 mg, 5.5 mmol, 2.0 eq.) in DMF (5 mL). The reaction mixture was stirred at 100° C. for 1 h, and then the solvent was evaporated under reduced pressure. The residue was partitioned between EtOAc (50 mL) and water (10 mL). The aqueous phase was extracted with EtOAc (50 mL) and the combined organic phase was washed with brine (10 mL), dried over magnesium sulfate, filtered and concentrated under reduced pressure. The residue was purified by column chromatography on silica gel (KP-Sil 40 g, eluting with 0-30% ethyl acetate in cyclohexane to give the title intermediate (601 mg, 75%) as a white solid. (ES, m/z): [M+H]⁺ 215. ¹H NMR (400 MHz, DMSO-d₆) δ 9.13 (s, 1H), 8.66 (s, 1H), 8.25 (s, 1H), 7.80 (s, 1H).

### Example 33—Synthesis of 1-(6-(Trifluoromethyl)Pyrimidin-4-yl)-Pyrazole-4-Sulfonyl Chloride

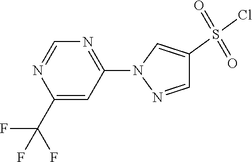 [SMILES: O=S(=O)(Cl)c1cnn(-c2cc(C(F)(F)F)ncn2)c1]

A solution of 4-(4-bromopyrazol-1-yl)-6-(trifluoromethyl)pyrimidine (200 mg, 0.7 mmol, 1.0 eq.), triisopropylsilanethiol (0.3 mL, 1.4 mmol, 2.0 eq.) and potassium bis(trimethylsilyl)amide (1 M in MeTHF, 0.7 mL, 0.7 mmol, 1.0 eq.) in toluene (5 mL) was degassed with a stream of nitrogen for 15 minutes. [1,1′-bis(diphenylphosphino)ferrocene]-dichloropalladium(II) (51 mg, 0.07 mmol, 0.1 eq.) was added and the mixture was allowed to stir at 110° C. for 1 h, after which it was filtered through Celite. The solid residue was washed with EtOAc (2×10 mL), and the filtrate was evaporated under reduced pressure to afford triisopropyl-[1-[6-(trifluoromethyl)pyrimidin-4-yl]pyrazol-4-yl]sulfanyl-silane, which was used directly in the next step, assuming quantitative yield.

A suspension of crude triisopropyl-[1-[6-(trifluoromethyl)pyrimidin-4-yl]pyrazol-4-yl]sulfanyl-silane (275 mg, 0.7 mmol, 1.0 eq.) in a mixture of acetic acid (5.00 mL), acetonitrile (3 mL) and water (1.0 mL) was treated with N-chlorosuccinimide (365 mg, 2.7 mmol, 4.0 eq.) at rt and stirred for 1.5 hour. Additional N-chlorosuccinimide (365 mg, 2.7 mmol, 4.0 eq) was added and the mixture was stirred for further 30 min. The mixture was then partitioned between EtOAc (25 mL) and water. The organic phase was washed with brine (5 mL), dried over magnesium sulfate, and concentrated under reduced pressure. The residue was purified by column chromatography (KP-Sil 40 g, 0-30% ethyl acetate in cyclohexane) to afford the title sulfonyl chloride (195 mg, 91%) as a white solid. (ES, m/z): [M+H]⁺ 293. ¹H NMR (400 MHz, CDCl₃) δ 9.31-9.29 (m, 1H), 9.28 (s, 1H), 8.36 (d, J=1.3 Hz, 1H), 8.27 (s, 1H).

### Example 34—Synthesis of 1-(4-Chloropyridin-2-yl)-Pyrazole-4-Sulfonyl Chloride

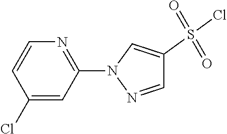 [SMILES: O=S(=O)(Cl)c1cnn(-c2cc(Cl)ccn2)c1]

Chlorosulfonic acid (6 mL) was added to 4-chloro-2-(pyrazol-1-yl)pyridine (300 mg, 1.7 mmol, 1 eq.) at 0° C. The resulting mixture was stirred for 4 h at 100° C. under nitrogen and then quenched with ice. The resulting mixture was extracted with DCM (3×20 mL), the combined organic layers were washed with brine (2×30 mL), dried over anhydrous Na₂SO₄, and concentrated under reduced pressure. This resulted in the title sulfonyl chloride (280 mg) as a light brown oil, which was used without further purification.

### Example 35—Synthesis of 1-(4-Methylpyridin-2-yl)-Pyrazole-4-Sulfonyl Chloride

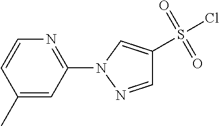 [SMILES: Cc1ccnc(-n2cc(S(=O)(=O)Cl)cn2)c1]

A solution of 4-Methyl-2-(pyrazol-1-yl)pyridine (220 mg, 1.4 mmol, 1 eq.) in chlorosulfonic acid (5 mL) was stirred overnight at 100° C. under nitrogen. The reaction was quenched with ice-cold water (10 mL), and then extracted with DCM (3×10 mL). The combined organic layers were washed with CH₂Cl₂ (3×10 mL), dried over anhydrous Na₂SO₄, and concentrated under reduced pressure. This resulted in the title sulfonyl chloride (150 mg, 42%) as a brown solid. (ES, m/z): [M+H]⁺ 258.

### Example 36—Synthesis of 4-(Benzylthio)-1H-1,2,3-Triazole

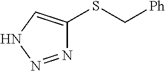 [SMILES: c1ccc(CSc2c[nH]nn2)cc1]

A solution of 4-(sodiosulfanyl)-1H-1,2,3-triazole (1 g, 8.1 mmol, 1 eq.) and benzyl bromide (1.4 g, 8.1 mmol, 1 eq.) in EtOH (10 mL) was stirred for 1 h at 0° C. under nitrogen. The resulting mixture was diluted with water (50 mL) and extracted with EtOAc (3×30 mL). The combined organic layers were washed with brine (2×50 mL), dried over anhydrous Na₂SO₄, and concentrated under reduced pressure to afford the title compound as a yellow oil, which was used without further purification. (ES, m/z): [M+H]⁺ 192.

### Example 37—Synthesis of 4-(4-(Benzylthio)-1H-1,2,3-Triazol-1-yl)-2-(Trifluoromethyl)Pyridine and 4-(4-(Benzylthio)-2H-1,2,3-Triazol-2-yl)-2-(Trifluoromethyl)Pyridine

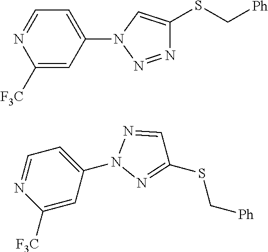 [SMILES: FC(F)(F)c1cc(-n2cc(SCc3ccccc3)nn2)ccn1 ; FC(F)(F)c1cc(-n2ncc(SCc3ccccc3)n2)ccn1]

A solution of 4-(benzylthio)-1H-1,2,3-triazole (3 g, 5.2 mmol, 1 eq.); 4-chloro-2-(trifluoromethyl)pyridine (2.85 g, 5.2 mmol, 1 eq.) and K₂CO₃ (6.5 g, 15.7 mmol, 3 eq.) in DMF (15 mL) was stirred for 24 h at 60° C. under nitrogen. The resulting mixture was diluted with water (50 mL) and extracted with EtOAc (3×30 mL). The combined organic layers were washed with brine (2×50 mL), dried over anhydrous Na₂SO₄, and concentrated under reduced pressure. The residue was purified by reverse flash chromatography with the following conditions: column: C18 silica gel; mobile phase, water in MeCN, 10% to 50% gradient in 10 min; detector: UV 254 nm to afford the title compounds as a white solids. 4-(4-(benzylthio)-1H-1,2,3-triazol-1-yl)-2-(trifluoromethyl)pyridine (1.16 g, 22%): (ES, m/z): [M+H]⁺ 337; ¹H NMR (400 MHz, DMSO-d₆) δ 4.26 (s, 2H), 7.18-7.35 (m, 5H), 8.27 (dd, J=5.4, 2.1 Hz, 1H), 8.41 (d, J=2.0 Hz, 1H), 8.96 (d, J=5.4 Hz, 1H), 9.22 (s, 1H). 4-(4-(benzylthio)-2H-1,2,3-triazol-2-yl)-2-(trifluoromethyl)pyridine (2.54 g, 48%): (ES, m/z): [M+H]⁺ 337; ¹H NMR (400 MHz, DMSO-d₆) δ 4.45 (s, 2H), 7.20-7.28 (m, 1H), 7.28-7.36 (m, 2H), 7.40-7.47 (m, 2H), 8.14˜8.23 (m, 2H), 8.34 (s, 1H), 8.89 (d, J=5.4 Hz, 1H).

### Example 38—Synthesis of 1-(2-(Trifluoromethyl)Pyridin-4-yl)-1H-1,2,3-Triazole-4-Sulfonyl Chloride

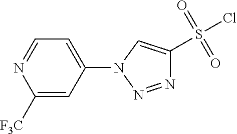 [SMILES: O=S(=O)(Cl)c1cn(-c2ccnc(C(F)(F)F)c2)nn1]

A solution of 4-(4-(benzylthio)-1H-1,2,3-triazol-1-yl)-2-(trifluoromethyl)pyridine (300 mg, 0.9 mmol, 1 eq.) and 1,3-dichloro-5,5-dimethylimidazolidine-2,4-dione (351 mg, 1.8 mmol, 2 eq.) in MeCN (2 mL), AcOH (0.075 mL) and H₂O (0.05 mL) was stirred for 2 h at 0° C. under air. The resulting mixture was diluted with water (20 mL) and extracted with EA (3×20 mL). The combined organic layers were washed with brine (2×20 mL), dried over anhydrous Na₂SO₄, and concentrated under reduced pressure to afford the title compound, which was used without further purification. (ES, m/z): [M+H]⁺ 313.

### Example 39—Synthesis of 2-(2-(Trifluoromethyl)Pyridin-4-yl)-2H-1,2,3-Triazole-4-Sulfonyl Chloride

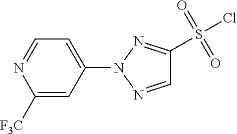 [SMILES: O=S(=O)(Cl)c1cnn(-c2ccnc(C(F)(F)F)c2)n1]

A solution of 4-(4-(benzylthio)-2H-1,2,3-triazol-2-yl)-2-(trifluoromethyl)pyridine (300 mg, 0.9 mmol, 1 eq.) and 1,3-dichloro-5,5-dimethylimidazolidine-2,4-dione (351 mg, 1.8 mmol, 2 eq.) in MeCN (2 mL), AcOH (0.075 mL) and H₂O (0.05 mL) was stirred for 2 h at 0° C. under air. The resulting mixture was diluted with water (20 mL) and extracted with EtOAc (3×20 mL). The combined organic layers were washed with brine (2×20 mL), dried over anhydrous Na₂SO₄, and concentrated under reduced pressure to afford the title sulfonyl chloride, which was used without further purification. (ES, m/z): [M+H]⁺ 313.

### Example 40—Synthesis of 4-(3-methyl-1H-Pyrazol-1-yl)-2-(Trifluoromethyl)Pyridine and 4-(5-Methyl-1H-Pyrazol-1-yl)-2-(Trifluoromethyl)Pyridine

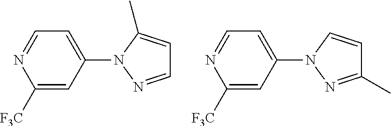

A solution of 3-methyl-2H-pyrazole (0.80 g, 9.7 mmol, 1 eq.) and NaH (0.94 g, 39.0, 4 eq.) in DMF (10 mL) was stirred for 0.5 h at 0° C. under nitrogen, and 4-chloro-2-(trifluoromethyl) pyridine (1.77 g, 9.7 mmol, 1 eq.) was added dropwise at rt. The mixture was stirred for 2 h at rt, and then quenched with water (50 mL) and extracted with EtOAc (3×50 mL). The combined organic layers were washed with brine (2×50 mL), dried over anhydrous Na₂SO₄, and concentrated under reduced pressure. The residue was purified by silica gel column chromatography and eluted with PE:EtOAc (3:1) to afford the title compounds as brown solids. 4-(5-methyl-1H-pyrazol-1-yl)-2-(trifluoromethyl) pyridine (500 mg, 23%): (ES, m/z): [M+H]⁺ 228. 4-(3-methyl-1H-pyrazol-1-yl)-2-(trifluoromethyl)pyridine (95 mg, 4%): (ES, m z): [M+H]⁺ 228.

### Example 41—Synthesis of 3-Methyl-1-(2-(Trifluoromethyl)Pyridin-4-yl)-1H-Pyrazole-4-Sulfonyl Chloride

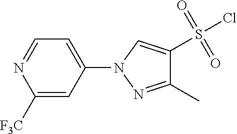 [SMILES: Cc1nn(-c2ccnc(C(F)(F)F)c2)cc1S(=O)(=O)Cl]

To chlorosulfonic acid (5 mL) was added 4-(3-methyl-1H-pyrazol-1-yl)-2-(trifluoromethyl) pyridine (85 mg, 0.4 mmol, 1 eq.) at 0° C. The reaction mixture was stirred for 24 h at 100° C., and then allowed to cool down to rt and was carefully quenched with H₂O/HCl (20 g ice/6.5 mL conc. HCl) at 0° C. The resulting mixture was extracted with DCM (3×10 mL). The combined organic layers were washed with water (3×10 mL), dried over anhydrous Na₂SO₄, and concentrated under reduced pressure. This resulted in the title sulfonyl chloride (86 mg, 71%) as a brown solid. (ES, m/z): [M+H]⁺ 326.

### Example 42—Synthesis of 5-Methyl-1-(2-(Trifluoromethyl)Pyridin-4-yl)-1H-Pyrazole-4-Sulfonyl Chloride

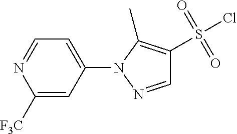 [SMILES: Cc1c(S(=O)(=O)Cl)cnn1-c1ccnc(C(F)(F)F)c1]

To chlorosulfonic acid (5 mL) was added 4-(5-methyl-1H-pyrazol-1-yl)-2-(trifluoromethyl)pyridine (500 mg, 2.2 mmol, 1 eq.) at 0° C. The reaction mixture was stirred for 24 h at 100° C. and then allowed to cool down to rt and was carefully quenched with H₂O/HCl (40 g ice/13 mL conc. HCl) at 0° C. The resulting mixture was extracted with DCM (3×20 mL). The combined organic layers were washed with water (3×20 mL), dried over anhydrous Na₂SO₄, and concentrated under reduced pressure to afford the title sulfonyl chloride (527 mg, 74%) as a brown solid. (ES, m/z): [M+H]⁺ 326.

### Example 43—Synthesis of 4-(4-((1H-Imidazol-1-yl)Sulfonyl)-1H-Pyrazol-1-yl)-2-(Trifluoromethyl)Pyridine

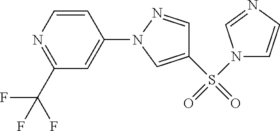 [SMILES: O=S(=O)(c1cnn(-c2ccnc(C(F)(F)F)c2)c1)n1ccnc1]

The title intermediate was obtained through general procedure F, and recrystallized from EtOAc/hexane (1/5) to give colourless crystals (2.6 g). (ES, m/z): [M+H]⁺ 344.

### Example 44—Synthesis of 3-Methyl-1-((1-(2-(Trifluoromethyl) Pyridin-4-yl)-1H-Pyrazol-4-yl)Sulfonyl)-1H-Imidazol-3-ium Iodide

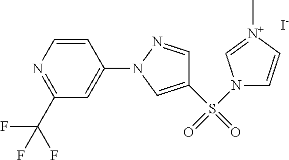 [SMILES: C[n+]1ccn(S(=O)(=O)c2cnn(-c3ccnc(C(F)(F)F)c3)c2)c1 ; [I-]]

The title intermediate was obtained through general procedure F (3.1 g, 99%) as an off-white solid. (ES, m/z): [M+H]⁺ 358.

### Example 45—Synthesis of 3 N-(6-Ethyl-1-Methyl-1H-Indazol-7-yl)-1-Trityl-1H-Pyrazole-4-Sulfonamide

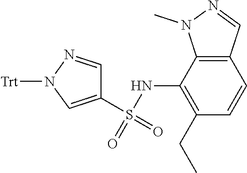

A solution of 1-trityl-1H-Pyrazole-4-sulfonyl chloride (4 g, 9.8 mmol, 1 eq.) and 6-ethyl-1-methylindazol-7-amine (2.1 g, 11.7 mmol, 1.2 eq.) in pyridine (40 mL) was stirred for 2 h at 80° C. under nitrogen. The resulting mixture was extracted with EtOAc (2×100 mL), the combined organic layers were washed with brine (200 mL), dried over anhydrous Na₂SO₄, and concentrated under reduced pressure. The residue was purified by reverse flash chromatography with the following conditions: column: C18 silica gel; mobile phase: MeCN in water (0.1% TFA), 10% to 50% gradient in 10 min; detector: UV 254 nm to afford the title compound (2.3 g, 43%) as a light brown solid. (ES, m/z): [M−1]⁻ 546; ¹H NMR (400 MHz, DMSO-d₆) δ 0.97 (t, J=7.5 Hz, 3H), 2.06 (d, J=57.8 Hz, 2H), 4.17 (s, 3H), 5.76 (s, 2H), 7.07-6.97 (m, 6H), 7.46-7.31 (m, 9H), 7.63 (d, J=8.3 Hz, 1H), 7.88 (d, J=0.7 Hz, 1H), 7.98 (s, 1H), 9.88 (s, 1H).

### Example 46—Synthesis of N-(6-Ethyl-1-Methyl-1H-Indazol-7-yl)-1H-Pyrazole-4-Sulfonamide

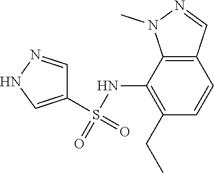 [SMILES: CCc1ccc2cnn(C)c2c1NS(=O)(=O)c1cn[nH]c1]

A solution of N-(6-Ethyl-1-Methyl-1H-Indazol-7-yl)-1-trityl-1H-Pyrazole-4-sulfonamide (6 g, 11 mmol, 1 eq.) and TFA (16.3 mL, 219 mmol, 20 eq.) in DCM (50 mL) was stirred for 2 h at rt. The resulting mixture was extracted with EtOAc (2×20 mL), the combined organic layers were washed with brine (4×40 mL), dried over anhydrous Na₂SO₄, and concentrated under reduced pressure. The residue was purified by reverse flash chromatography with the following conditions: column: C18 silica gel; mobile phase: MeCN in water (0.1% TFA), 10% to 50% gradient in 10 min; detector: UV 254 nm to afford the title compound (2 g, 60%) as an off-white solid. (ES, m/z): [M+1]⁺306; ¹H NMR (400 MHz, DMSO-d₆) δ 0.88 (d, J=7.6 Hz, 3H), 2.19 (q, J=7.6 Hz, 2H), 4.22 (s, 4H), 6.98 (d, J=8.4 Hz, 1H), 7.62 (d, J=8.4 Hz, 2H), 7.98 (s, 1H), 8.12 (s, 1H), 9.75 (s, 1H), 13.59 (s, 1H).

### Example 47—Synthesis of 3N-(6-Ethyl-1-Methyl-1H-Indazol-7-yl)-1-(5-(Trifluoromethyl)Pyridin-2-yl)-1H-Pyrazole-4-Sulfonamide (I-1)

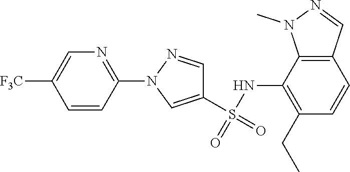 [SMILES: CCc1ccc2cnn(C)c2c1NS(=O)(=O)c1cnn(-c2ccc(C(F)(F)F)cn2)c1]

The title compound was obtained using general procedure A as a light-green solid (1.3 g, 27%). (ES, m/z): [M+H]⁺ 451; ¹H NMR (400 MHz, DMSO-d₆) δ 0.85 (t, J=7.6 Hz, 3H), 2.27 (q, J=7.6 Hz, 2H), 4.26 (s, 3H), 6.99 (d, J=8.4 Hz, 1H), 7.65 (d, J=8.4 Hz, 1H), 8.00 (s, 1H), 8.18-8.13 (m, 2H), 8.49 (dd, J=8.8 Hz, 6.8 Hz, 1H), 8.78 (s, 1H), 8.96 (s, 1H), 10.16 (s, 1H).

### Example 48—Synthesis of N-(6-Ethyl-1-Methyl-1H-Indazol-7-yl)-1-(2-(Trifluoromethyl) Pyridin-4-yl)-1H-Pyrazole-4-Sulfonamide (I-2)

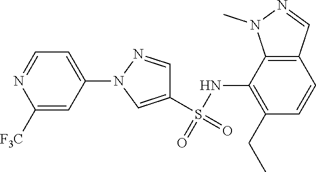 [SMILES: CCc1ccc2cnn(C)c2c1NS(=O)(=O)c1cnn(-c2ccnc(C(F)(F)F)c2)c1]

The title compound was obtained using general procedure A as an off-white solid (1.0 g, 28%). (ES, m/z): [M+H]⁺ 451; ¹H NMR (400 MHz, DMSO-d₆) δ 1.03 (t, J=7.6 Hz, 3H), 2.27 (q, J=7.6 Hz, 2H), 4.42 (s, 3H), 6.49 (s, 1H), 6.96-7.02 (m, 1H), 7.68-7.80 (m, 2H), 7.91 (s, 1H), 8.00-8.08 (s, 2H), 8.20 (s, 1H), 8.86 (d, J=5.2 Hz, 1H).

### Example 49—Synthesis of N-(6-Ethyl-1-Methyl-1H-Indazol-7-yl)-1-(5-(Trifluoromethyl) Pyridin-3-yl)-1H-Pyrazole-4-Sulfonamide (I-3)

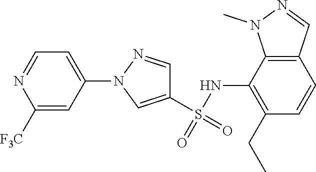 [SMILES: CCc1ccc2cnn(C)c2c1NS(=O)(=O)c1cnn(-c2ccnc(C(F)(F)F)c2)c1]

The title compound was obtained using general procedure A as a white solid (2.3 mg, 5%). (ES, m/z): [M+H]⁺ 451; ¹H NMR (400 MHz, DMSO-d₆) δ 0.87 (t, J=7.6, 3H), 2.33 (q, J=7.6 Hz, 2H), 4.26 (s, 3H), 7.00 (d, J=8.0 Hz, 1H), 7.62 (d, J=8.4 Hz, 1H), 7.99 (s, 1H), 8.09 (s, 1H), 8.74 (s, 1H), 9.10 (s, 1H), 9.23 (s, 1H), 9.46 (d, J=2.4 Hz, 1H), 10.12 (s, 1H).

### Example 50—Synthesis of 3N-(6-Cyclopropyl-1-Methyl-1H-Indazol-7-yl)-1-(5-(Trifluoromethyl)Pyridin-3-yl)-1H-Pyrazole-4-Sulfonamide (I-17)

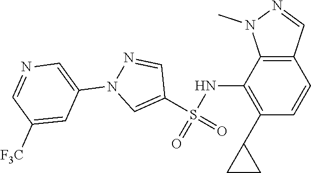 [SMILES: Cn1ncc2ccc(C3CC3)c(NS(=O)(=O)c3cnn(-c4cncc(C(F)(F)F)c4)c3)c21]

The title compound was obtained using general procedure A as a white solid (4.0 mg, 3%). (ES, m/z): [M+H]⁺ 463; ¹H NMR (400 MHz, DMSO-d₆) δ 0.52 (m, 3H), 0.89 (s, 1H), 1.90 (m, 1H), 4.31 (s, 3H), 6.32 (d, J=8.4 Hz, 1H), 7.48 (d, J=8.4 Hz, 1H), 7.92 (s, 1H), 8.03 (s, 1H), 8.72 (s, 1H), 9.00 (s, 1H), 9.09 (s, 1H), 9.45 (s, 1H).

### Example 51—Synthesis of N-(6-Methoxy-1-Methyl-1H-Indazol-7-yl)-1-(4-(Trifluoromethyl)Pyridin-2-yl)-1H-Pyrazole-4-Sulfonamide (I-5)

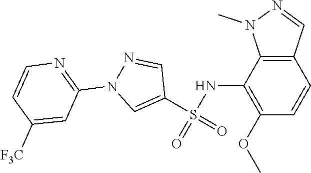 [SMILES: COc1ccc2cnn(C)c2c1NS(=O)(=O)c1cnn(-c2cc(C(F)(F)F)ccn2)c1]

The title compound was obtained using general procedure A as a white solid (10 mg, 3%). (ES, m/z): [M+H]⁺ 453; ¹H NMR (400 MHz, DMSO-d₆) δ 3.35 (s, 3H), 4.25 (s, 3H), 6.88 (d, J=9.2 Hz, 1H), 7.67 (d, J=8.8 Hz, 1H), 7.88 (dd, J=1.6, 5.2 Hz, 1H), 7.98 (s, 1H), 8.08 (s, 1H), 8.20 (s, 1H), 8.78 (s, 1H), 8.83 (d, J=5.2 Hz, 1H), 9.83 (s, 1H).

### Example 52—Synthesis of N-(6-Methoxy-1-Methyl-1H-Indazol-7-yl)-1-(2-(Trifluoromethyl)Pyridin-4-yl)-1H-Pyrazole-4-Sulfonamide (I-4)

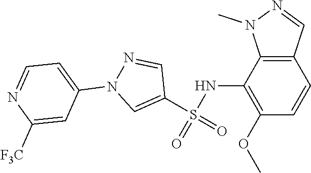 [SMILES: COc1ccc2cnn(C)c2c1NS(=O)(=O)c1cnn(-c2ccnc(C(F)(F)F)c2)c1]

The title compound was obtained using general procedure A as a grey solid (947 mg, 25%). (ES, m/z): [M+H]⁺ 453; ¹H NMR (400 MHz, methanol-d₄) δ 3.31 (s, 3H), 4.24 (s, 3H), 6.75 (d, J=8.9 Hz, 1H), 7.57 (d, J=8.9 Hz, 1H), 7.83 (s, 1H), 7.89 (d, J=0.5 Hz, 1H), 7.99 (dd, J=5.5, 2.2 Hz, 1H), 8.24-8.19 (m, 1H), 8.69 (d, J=8.9 Hz, 1H), 8.75 (s, 1H).

### Example 53—Synthesis of N-(6-Methoxy-1-Methyl-1H-Indazol-7-yl)-1-(5-(Trifluoromethyl)Pyridin-2-yl)-1H-Pyrazole-4-Sulfonamide (I-22)

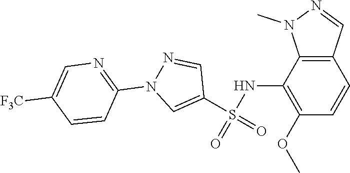 [SMILES: COc1ccc2cnn(C)c2c1NS(=O)(=O)c1cnn(-c2ccc(C(F)(F)F)cn2)c1]

The title compound was obtained using general procedure A as a white solid (56 mg, 14%). (ES, m/z): [M+H]⁺ 453; ¹H NMR (400 MHz, CDCl₃) δ 3.41 (s, 3H), 4.40 (s, 3H), 6.57 (s, 1H), 6.66 (d, J=8.8 Hz, 1H), 7.61 (d, J=8.8 Hz, 1H), 7.94 (s, 1H), 7.76 (s, 1H), 8.10 (d, J=1.6 Hz, 2H), 8.69 (s, 1H), 8.74 (s, 1H).

### Example 54—Synthesis of N-(6-Methoxy-1-Methyl-1H-Indazol-7-yl)-1-(5-(Trifluoromethyl)Pyridin-3-yl)-1H-Pyrazole-4-Sulfonamide (I-23)

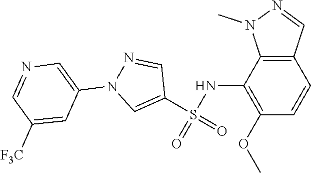 [SMILES: COc1ccc2cnn(C)c2c1NS(=O)(=O)c1cnn(-c2cncc(C(F)(F)F)c2)c1]

The title compound was obtained using general procedure A as a white solid (10 mg, 8%). (ES, m/z): [M+H]⁺ 453; ¹H NMR (400 MHz, DMSO-d₆) δ 3.36 (s, 3H), 4.26 (s, 3H), 6.89 (d, J=8.9 Hz, 1H), 7.67 (d, J=8.8 Hz, 1H), 7.98 (s, 1H), 8.08 (s, 1H), 8.75 (d, J=2.2 Hz, 1H), 9.01 (d, J=1.7 Hz, 1H), 9.18 (s, 1H), 9.47 (d, J=2.4 Hz, 1H), 9.78 (s, 1H).

### Example 55—Synthesis of N-(6-Methoxy-1-Methyl-1H-Indazol-7-yl)-1-(6-(Trifluoromethyl)Pyrimidin-4-yl)-1H-Pyrazole-4-Sulfonamid (I-24)

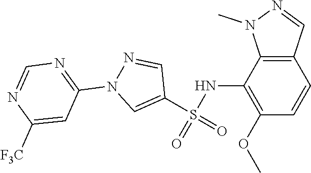 [SMILES: COc1ccc2cnn(C)c2c1NS(=O)(=O)c1cnn(-c2cc(C(F)(F)F)ncn2)c1]

The title compound was obtained using general procedure A as a white solid (5 mg, 8%). (ES, m/z): [M+H]⁺ 454; ¹H NMR (400 MHz, DMSO-d₆) δ 3.37 (s, 3H), 4.24 (s, 3H), 6.88 (d, J=8.8 Hz, 1H), 7.68 (d, J=8.8 Hz, 1H), 7.99 (s, 1H), 8.22 (s, 1H), 8.33 (d, J=1.2 Hz, 1H), 8.89 (d, J=1.2 Hz, 1H), 9.39 (s, 1H), 10.03 (s, 1H).

### Example 56—Synthesis of N-(6-Ethoxy-1-Methyl-1H-Indazol-7-yl)-1-(2-(Trifluoromethyl)Pyridin-4-yl)-1H-Pyrazole-4-Sulfonamide (I-9)

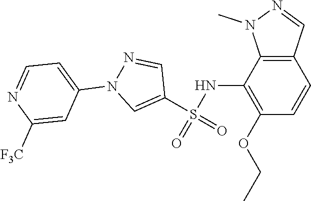 [SMILES: CCOc1ccc2cnn(C)c2c1NS(=O)(=O)c1cnn(-c2ccnc(C(F)(F)F)c2)c1]

The title compound was obtained using general procedure A as a white solid (52 mg, 21%). (ES, m/z): [M+H]⁺ 467; ¹H NMR (400 MHz, DMSO-d₆) δ 0.87 (t, J=6.9 Hz, 3H), 3.71 (d, J=7.3 Hz, 2H), 4.25 (s, 3H), 6.88 (d, J=8.9 Hz, 1H), 7.66 (d, J=8.9 Hz, 1H), 7.97 (s, 1H), 8.09 (s, 1H), 8.27 (dd, J=2.1, 5.4 Hz, 1H), 8.47 (d, J=2.1 Hz, 1H), 8.88 (d, J=5.5 Hz, 1H), 9.34 (s, 1H), 9.77 (s, 1H).

### Example 57—Synthesis of N-(6-Isopropoxy-1-Methyl-1H-Indazol-7-yl)-1-(2-(Trifluoromethyl)Pyridin-4-yl)-1H-Pyrazole-4-Sulfonamide (I-10)

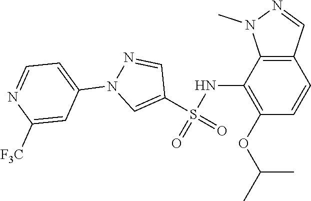 [SMILES: CC(C)Oc1ccc2cnn(C)c2c1NS(=O)(=O)c1cnn(-c2ccnc(C(F)(F)F)c2)c1]

The title compound was obtained using general procedure A as a white solid (48 mg, 17%). (ES, m/z): [M+H]⁺ 481; ¹H NMR (400 MHz, DMSO-d₆) δ 0.88 (d, J=5.7 Hz, 6H), 4.27 (s, 3H), 4.41 (m, 1H), 6.86 (d, J=9.0 Hz, 1H), 7.63 (d, J=8.9 Hz, 1H), 8.14 (s, 1H), 7.96 (s, 1H), 8.28 (dd, J=5.5, 2.1 Hz, 1H), 8.47 (d, J=2.1 Hz, 1H), 8.88 (d, J=5.5 Hz, 1H), 9.34 (s, 1H), 9.69 (s, 1H).

### Example 58—Synthesis of N-(6-(Cyanomethyl)-1-Methyl-1H-Indazol-7-yl)-1-(2-(Trifluoromethyl)Pyridin-4-yl)-1H-Pyrazole-4-Sulfonamide (I-6)

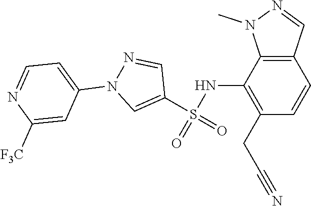 [SMILES: Cn1ncc2ccc(CC#N)c(NS(=O)(=O)c3cnn(-c4ccnc(C(F)(F)F)c4)c3)c21]

The title compound was obtained using general procedure B as a white solid (18 mg, 6%). (ES, m/z): [M+H]⁺ 462; ¹H NMR (400 MHz, DMSO-d₆) δ 3.72 (s, 2H), 4.23 (s, 3H), 7.13 (d, J=8.3 Hz, 1H), 7.76 (d, J=8.3 Hz, 1H), 8.09 (s, 1H), 8.18 (s, 1H), 8.24 (dd, =2.1, 5.5 Hz, 1H), 8.44 (d, J=2.1 Hz, 1H), 8.87 (d, J=5.5 Hz, 1H), 9.36 (s, 1H), 10.40 (s, 1H).

### Example 59—Synthesis of N-(6-(Dimethylamino)-1-Methyl-1H-Indazol-7-yl)-1-(2-(Trifluoromethyl)Pyridin-4-yl)-1H-Pyrazole-4-Sulfonamide (I-7)

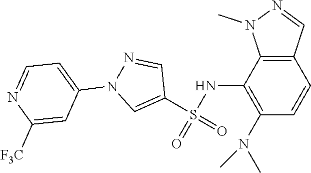 [SMILES: CN(C)c1ccc2cnn(C)c2c1NS(=O)(=O)c1cnn(-c2ccnc(C(F)(F)F)c2)c1]

The title compound was obtained using general procedure A as a white solid (73 mg, 19%). (ES, m/z): [M+H]⁺ 466; ¹H NMR (400 MHz, DMSO-d₆) δ 2.36 (s, 6H), 4.28 (s, 3H), 6.85 (d, J=8.8 Hz, 1H), 7.60 (d, J=8.8 Hz, 1H), 7.93 (s, 1H), 8.17 (s, 1H), 8.30 (dd, J=5.6, 2.0 Hz, 1H), 8.49 (d, J=2.0 Hz, 1H), 8.88 (d, J=5.6 Hz, 1H), 9.35 (s, 1H), 9.72 (s, 1H).

### Example 60—Synthesis of N-(6-((Tert-Butyldimethylsilyl)Ethynyl)-1-Methylindazol-7-yl)-1-(2-(Trifluoromethyl)Pyridin-4-yl)-Pyrazole-4-Sulfonamide

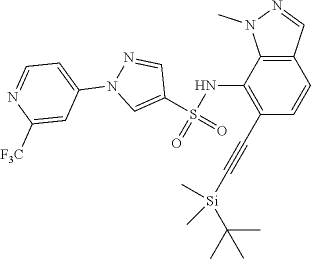 [SMILES: Cn1ncc2ccc(C#C[Si](C)(C)C(C)(C)C)c(NS(=O)(=O)c3cnn(-c4ccnc(C(F)(F)F)c4)c3)c21]

The title compound was obtained using general procedure B as a yellow solid (100 mg, 17%). (ES, m/z): [M+H]⁺ 561.

### Example 61—Synthesis of N-(6-Ethynyl-1-Methylindazol-7-yl)-1-(2-(Trifluoromethyl) Pyridin-4-yl)-Pyrazole-4-Sulfonamide (I-8)

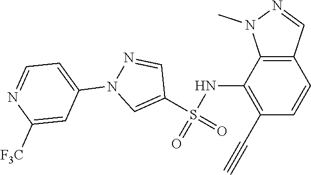 [SMILES: C#Cc1ccc2cnn(C)c2c1NS(=O)(=O)c1cnn(-c2ccnc(C(F)(F)F)c2)c1]

A solution of N-{6-[2-(tert-butyldimethylsilyl)ethynyl]-1-methylindazol-7-yl}-1-[2-(trifluoromethyl)pyridin-4-yl]pyrazole-4-sulfonamide (90 mg, 0.16 mmol, 1.0 eq.) in TEA (2 mL) was stirred for 16 h at 70° C. The resulting mixture was diluted with water (5 mL), and extracted with EtOAc (3×5 mL). The combined organic layers were washed with brine (2×10 mL), dried over anhydrous Na₂SO₄, and concentrated under reduced pressure. The residue was purified by reverse flash chromatography with the following conditions: column: C18 silica gel; mobile phase: MeCN in water (10 mmol/L NH₄HCO₃), 10% to 50% gradient in 10 min; detector: UV 254 nm to afford the title sulfonamide (45 mg, 62%) as a white solid. (ES, m/z): [M+H]⁺ 447; ¹H NMR (400 MHz, DMSO-d₆) δ 3.78 (s, 1H), 4.32 (s, 3H), 7.05 (d, J=8.3 Hz, 1H), 7.62 (s, 1H), 7.99 (s, 1H), 8.08 (s, 1H), 8.25 (dd, J=5.5, 2.1 Hz, 1H), 8.45 (d, J=2.0 Hz, 1H), 8.86 (d, J=5.5 Hz, 1H), 9.26 (s, 1H).

### Example 62—Synthesis of N-(6-Ethyl-1-Methyl-1H-Indazol-7-yl)-1-(2-(Trifluoromethyl) Pyrimidin-4-yl)-1H-Pyrazole-4-Sulfonamide (I-11)

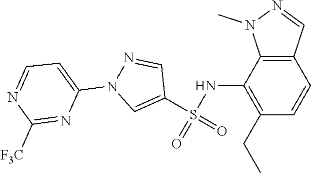 [SMILES: CCc1ccc2cnn(C)c2c1NS(=O)(=O)c1cnn(-c2ccnc(C(F)(F)F)n2)c1]

The title compound was obtained using general procedure A as an off-white solid (3 mg, 6%). (ES, m/z): [M+H]⁺ 452; ¹H NMR (400 MHz, DMSO-d₆) δ 0.90 (t, J=7.5 Hz, 3H), 2.36-2.28 (m, 2H), 4.27-4.26 (m, 3H), 7.01-6.97 (m, 1H), 7.63-7.59 (m, 1H), 7.99 (s, 1H), 8.20-8.18 (m, 1H), 8.28 (d, J=5.6 Hz, 1H), 8.79 (s, 1H), 9.21 (d, J=5.6 Hz, 1H), 10.37 (s, 1H).

### Example 63—Synthesis of N-(6-Ethyl-1-Methyl-1H-Indazol-7-yl)-1-(2-(Trifluoromethyl) Pyridin-2-yl)-1H-Pyrazole-4-Sulfonamide (I-12)

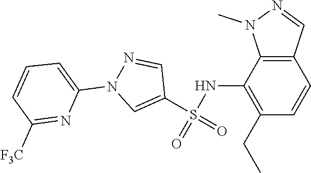 [SMILES: CCc1ccc2cnn(C)c2c1NS(=O)(=O)c1cnn(-c2cccc(C(F)(F)F)n2)c1]

The title compound was obtained using general procedure A as an off-white solid (20 mg, 26%). (ES, m/z): [M+H]⁺ 451; ¹H NMR (400 MHz, DMSO-d₆) δ 0.88 (t, J=7.5 Hz, 3H), 2.36-2.29 (m, 2H), 4.27 (s, 3H), 7.02-6.99 (m, 1H), 7.68-7.64 (m, 1H), 8.03-8.00 (m, 2H), 8.12 (s, 1H), 8.31-8.28 (m, 1H), 8.39 (t, J=8.0 Hz, 1H), 8.73 (s, 1H), 10.12 (s, 1H).

### Example 64—Synthesis of N-(1-Methyl-1H-Indazol-7-yl)-1-(6-(Trifluoromethyl)Pyridin-2-yl)-1H-Pyrazole-4-Sulfonamide (I-13)

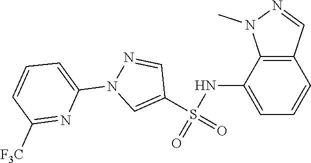 [SMILES: Cn1ncc2cccc(NS(=O)(=O)c3cnn(-c4cccc(C(F)(F)F)n4)c3)c21]

The title compound was obtained using general procedure A as an off-white solid (29 mg, 34%). (ES, m/z): [M+H]⁺ 423; ¹H NMR (400 MHz, DMSO-d₆) δ 4.32-4.31 (m, 3H), 6.76-6.73 (m, 1H), 6.98 (t, J=7.7 Hz, 1H), 7.72-7.68 (m, 1H), 8.01 (d, J=7.4 Hz, 1H), 8.11-8.08 (m, 2H), 8.32-8.29 (m, 1H), 8.39 (t, J=8.0 Hz, 1H), 8.85 (s, 1H), 10.06 (s, 1H).

### Example 65—Synthesis of N-(6-Ethyl-1-Methyl-1H-Indazol-7-yl)-1-(4-(Trifluoromethyl) Pyridin-2-yl)-1H-Pyrazole-4-Sulfonamide (I-14)

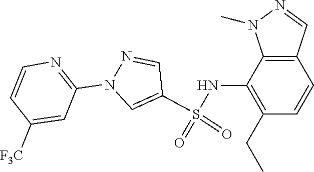 [SMILES: CCc1ccc2cnn(C)c2c1NS(=O)(=O)c1cnn(-c2cc(C(F)(F)F)ccn2)c1]

The title compound was obtained using general procedure A as an off-white solid (30 mg, 31%). (ES, m/z): [M+H]⁺ 451; ¹H NMR (400 MHz, DMSO-d₆) δ 0.88 (t, J=7.9 Hz, 3H), 2.29-2.24 (m, 2H), 4.27 (s, 3H), 7.01 (d, J=9.8 Hz, 1H), 7.66 (d, J=12.3 Hz, 1H), 7.91-7.89 (m, 1H), 8.01 (s, 1H), 8.11 (s, 1H), 8.21 (s, 1H), 8.80 (s, 1H), 8.83 (d, J=5.1 Hz, 1H), 10.18-10.12 (m, 1H).

### Example 66—Synthesis of N-(1-Methyl-1H-Indazol-7-yl)-1-(4-(Trifluoromethyl)Pyridin-2-yl)-1H-Pyrazole-4-Sulfonamide (I-15)

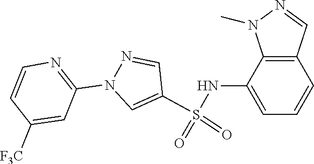 [SMILES: Cn1ncc2cccc(NS(=O)(=O)c3cnn(-c4cc(C(F)(F)F)ccn4)c3)c21]

The title compound was obtained using general procedure A as an off-white solid (51 mg, 59%). (ES, m/z): [M+H]⁺ 423; ¹H NMR (400 MHz, DMSO-d₆) δ 4.31 (s, 3H), 6.76 (d, J=6.8 Hz, 1H), 6.96 (t, J=7.7 Hz, 1H), 7.63 (t, J=7.1 Hz, 1H), 7.89 (d, J=4.4 Hz, 1H), 8.09 (d, J=11.5 Hz, 2H), 8.21 (s, 1H), 8.87-8.82 (m, 2H), 10.13 (s, 1H).

### Example 67—Synthesis of N-(1-Methyl-1H-Indazol-7-yl)-1-(2-(Trifluoromethyl)Pyridin-4-yl)-1H-Pyrazole-4-Sulfonamidemide (I-16)

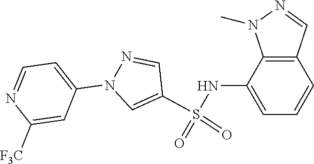 [SMILES: Cn1ncc2cccc(NS(=O)(=O)c3cnn(-c4ccnc(C(F)(F)F)c4)c3)c21]

The title compound was obtained using general procedure A as an off-white solid (60 mg, 69%). (ES, m/z): [M+H]⁺ 423; ¹H NMR (400 MHz, DMSO-d₆) δ 4.32-4.31 (m, 3H), 6.77 (d, J=7.2 Hz, 1H), 6.99 (t, J=7.7 Hz, 1H), 7.70-7.65 (m, 1H), 8.08 (s, 1H), 8.17-8.16 (m, 1H), 8.29 (dd, J=2.0, 5.5 Hz, 1H), 8.48 (d, J=2.0 Hz, 1H), 8.89 (d, J=5.5 Hz, 1H), 9.38 (s, 1H), 10.17-10.08 (m, 1H).

### Example 68—Synthesis of N-(6-Cyclopropyl-1-Methyl-1H-Indazol-7-yl)-1-(4-(Trifluoromethyl)Pyridin-2-yl)-1H-Pyrazole-4-Sulfonamide (I-17)

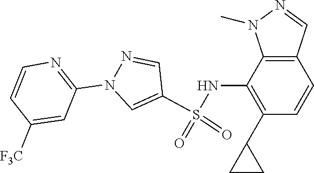 [SMILES: Cn1ncc2ccc(C3CC3)c(NS(=O)(=O)c3cnn(-c4cc(C(F)(F)F)ccn4)c3)c21]

The title compound was obtained using general procedure A as an off-white solid (19 mg, 26%). (ES, m/z): [M+H]⁺ 463; ¹H NMR (400 MHz, DMSO-d₆) δ 0.42-0.41 (m, 4H), 1.86-1.79 (m, 1H), 4.30 (s, 3H), 6.35 (d, J=8.6 Hz, 1H), 7.57 (d, J=8.3 Hz, 1H), 7.89-7.87 (m, 1H), 7.96 (s, 1H), 8.07 (s, 1H), 8.18 (s, 1H), 8.67 (s, 1H), 8.81 (d, J=5.1 Hz, 1H), 10.24 (s, 1H).

### Example 69—Synthesis of N-(6-Ethyl-1-Methyl-1H-Indazol-7-yl)-1-(6-(Trifluoromethyl) Pyrimidin-4-yl)-1H-Pyrazole-4-Sulfonamide (I-19)

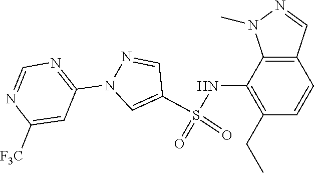 [SMILES: CCc1ccc2cnn(C)c2c1NS(=O)(=O)c1cnn(-c2cc(C(F)(F)F)ncn2)c1]

The title compound was obtained using general procedure A as an off-white solid (11 mg, 16%). (ES, m/z): [M+H]⁺ 452; ¹H NMR (400 MHz, DMSO-d₆) δ 0.89 (t, J=7.5 Hz, 3H), 2.34-2.23 (m, 2H), 4.24 (s, 3H), 7.01-6.98 (m, 1H), 7.66-7.63 (m, 1H), 8.00 (s, 1H), 8.22 (s, 1H), 8.34 (d, J=1.0 Hz, 1H), 8.91 (s, 1H), 9.39 (s, 1H), 10.24-10.21 (m, 1H).

### Example 70—Synthesis of N-(6-Cyclopropyl-1-Methyl-1H-Indazol-7-yl)-1-(5-(Trifluoromethyl)Pyridin-2-yl)-1H-Pyrazole-4-Sulfonamide (I-20)

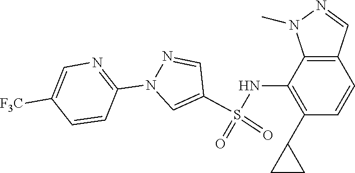 [SMILES: Cn1ncc2ccc(C3CC3)c(NS(=O)(=O)c3cnn(-c4ccc(C(F)(F)F)cn4)c3)c21]

The title compound was obtained using general procedure A as an off-white solid (11 mg, 15%). (ES, m/z): [M+H]⁺ 463; ¹H NMR (400 MHz, DMSO-d₆) δ 0.42-0.41 (m, 4H), 1.85-1.78 (m, 1H), 4.30 (s, 3H), 6.34 (d, J=8.6 Hz, 1H), 7.56 (d, J=8.3 Hz, 1H), 7.96 (s, 1H), 8.09 (s, 1H), 8.16 (d, J=8.6 Hz, 1H), 8.48 (dd, J=2.1, 8.7 Hz, 1H), 8.65 (s, 1H), 8.94 (s, J=0.8 Hz, 1H), 10.26 (s, 1H).

### Example 71—Synthesis of N-(6-cyclopropyl-1-Methyl-1H-Indazol-7-yl)-1-(2-(Trifluoromethyl)Pyridin-4-yl)-1H-Pyrazole-4-Sulfonamide (I-21)

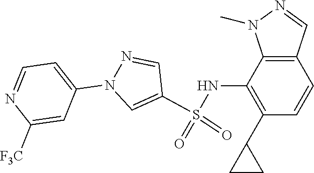 [SMILES: Cn1ncc2ccc(C3CC3)c(NS(=O)(=O)c3cnn(-c4ccnc(C(F)(F)F)c4)c3)c21]

The title compound was obtained using general procedure A as an off-white solid (16 mg, 23%). (ES, m/z): [M+H]⁺ 463; ¹H NMR (400 MHz, DMSO-d₆) δ 0.41-0.38 (m, 4H), 1.86-1.77 (m, 1H), 4.31 (s, 3H), 6.34 (d, J=8.6 Hz, 1H), 7.54 (d, J=8.6 Hz, 1H), 7.94 (s, 1H), 8.08 (s, 1H), 8.25 (dd, J=2.0, 5.6 Hz, 1H), 8.45 (d, J=1.8 Hz, 1H), 8.87 (d, J=5.6 Hz, 1H), 9.27 (s, 1H), 10.17 (s, 1H).

### Example 72—synthesis of N-(6-Methoxy-1-Methyl-1H-Indazol-7-yl)-N-methyl-1-(2-(Trifluoromethyl)Pyridin-4-yl)-1H-Pyrazole-4-Sulfonamide (I-25)

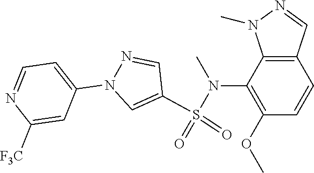 [SMILES: COc1ccc2cnn(C)c2c1N(C)S(=O)(=O)c1cnn(-c2ccnc(C(F)(F)F)c2)c1]

To a stirred mixture of N-(6-methoxy-1-methylindazol-7-yl)-1-[2-(trifluoromethyl) pyridin-4-yl]pyrazole-4-sulfonamide (100 mg, 0.16 mmol, 1 eq.) and K₂CO₃ (45 mg, 0.3 mmol, 2 eq.) in DMF (1 mL) was added Mel (0.10 mL, 1.6 mmol, 10 eq.) dropwise at rt under nitrogen. The resulting mixture was stirred for 2 h at rt, and then diluted with water (10 mL), and extracted with EtOAc (3×10 mL). The combined organic layers were washed with brine (10 mL), dried over Na₂SO₄, and concentrated under reduced pressure. The residue was purified by Prep-HPLC, affording the title compound (28 mg, 37%) as a white solid. (ES, m/z): [M+H]⁺ 467; ¹H NMR (400 MHz, DMSO-d₆) δ 3.31 (s, 3H), 3.40 (s, 3H), 4.22 (s, 3H), 6.94 (d, J=8.9 Hz, 1H), 7.74 (d, J=8.9 Hz, 1H), 8.00 (s, 1H), 8.26 (s, 1H), 8.33 (dd, J=5.5, 2.1 Hz, 1H), 8.55 (d, J=2.1 Hz, 1H), 8.91 (d, J=5.5 Hz, 1H), 9.55 (s, 1H).

### Example 73—Synthesis of N-(6-Methoxy-1-Methyl-1H-Indazol-7-yl)-N-Ethyl-1-(2-(Trifluoromethyl)Pyridin-4-yl)-1H-Pyrazole-4-Sulfonamide (I-26)

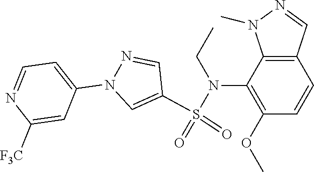 [SMILES: CCN(c1c(OC)ccc2cnn(C)c12)S(=O)(=O)c1cnn(-c2ccnc(C(F)(F)F)c2)c1]

To a stirred mixture of N-(6-methoxy-1-methylindazol-7-yl)-1-[2-(trifluoromethyl) pyridin-4-yl]pyrazole-4-sulfonamide (150 mg, 0.3 mmol, 1 eq.) and K₂CO₃ (92 mg, 0.7 mmol, 2 eq.) in DMF (1 mL) was added iodoethane (0.53 mL, 6.6 mmol, 20 eq.) dropwise at rt under nitrogen. The resulting mixture was stirred for 2 h at rt, and then diluted with water (10 mL), and extracted with EtOAc (3×10 mL). The combined organic layers were washed with brine (10 mL), dried over Na₂SO₄, and concentrated under reduced pressure. The residue was purified by Prep-HPLC, affording the title compound (58 mg, 36%) as an off-white solid. (ES, m/z): [M+H]⁺ 481; ¹H NMR (400 MHz, DMSO-d₆) δ 1.06 (t, J=7.2 Hz, 3H), 3.37 (s, 3H), 3.50 (m, 1H), 3.98 (m, 1H), 4.24 (s, 3H), 6.94 (d, J=8.9 Hz, 1H), 7.75 (d, J=8.8 Hz, 1H), 8.01 (s, 1H), 8.23 (s, 1H), 8.32 (dd, J=5.5, 2.1 Hz, 1H), 8.53 (d, J=2.1 Hz, 1H), 8.90 (d, J=5.5 Hz, 1H), 9.53 (s, 1H).

### Example 74—Synthesis of N-(6-Ethyl-1-Methyl-1H-Indazol-7-yl)-1-(2-(Trifluoromethyl) Pyridin-4-yl)-1H-1,2,3-Triazole-4-Sulfonamide (I-27)

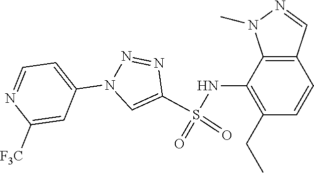 [SMILES: CCc1ccc2cnn(C)c2c1NS(=O)(=O)c1cn(-c2ccnc(C(F)(F)F)c2)nn1]

The title compound was obtained using general procedure A as a white solid (47 mg, 16%). (ES, m/z): [M+H]⁺ 452; ¹H NMR (400 MHz, DMSO-d₆) δ 0.85 (t, J=7.5 Hz, 3H), 2.08-2.24 (m, 2H), 4.26 (s, 3H), 6.99 (d, J=8.4 Hz, 1H), 7.65 (d, J=8.3 Hz, 1H), 8.01 (s, 1H), 8.42 (dd, J=5.5, 2.0 Hz, 1H), 8.60 (d, J=2.1 Hz, 1H), 9.01 (d, J=5.5 Hz, 1H), 9.74 (s, 1H), 10.71 (s, 1H).

### Example 75—Synthesis of N-(6-Ethyl-1-Methyl-1H-Indazol-7-yl)-2-(2-(Trifluoromethyl) Pyridin-4-yl)-2H-1,2,3-Triazole-4-Sulfonamide (I-28)

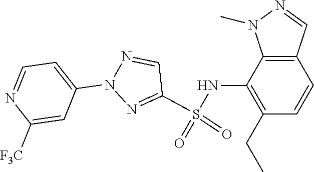 [SMILES: CCc1ccc2cnn(C)c2c1NS(=O)(=O)c1cnn(-c2ccnc(C(F)(F)F)c2)n1]

The title compound was obtained using general procedure A as a white solid (67 mg, 23%). (ES, m/z): [M+H]⁺ 452; ¹H NMR (400 MHz, DMSO-d₆) δ 0.84 (t, J=7.5 Hz, 3H), 2.23 (q, J=7.5 Hz, 2H), 4.22 (s, 3H), 6.99 (d, J=8.3 Hz, 1H), 7.66 (d, J=8.3 Hz, 1H), 8.01 (s, 1H), 8.33-8.25 (m, 2H), 8.68 (s, 1H), 9.02 (d, J=5.3 Hz, 1H).

### Example 76—Synthesis of N-(6-Ethyl-1-Methyl-1H-Indazol-7-yl)-3-Methyl-1-(2-(Trifluoromethyl)Pyridin-4-yl)-1H-Pyrazole-4-Sulfonamide (I-29)

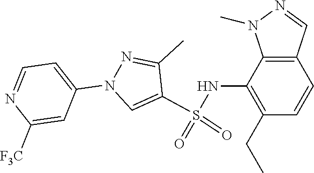 [SMILES: CCc1ccc2cnn(C)c2c1NS(=O)(=O)c1cn(-c2ccnc(C(F)(F)F)c2)nc1C]

The title compound was obtained using general procedure A as an off-white solid (5 mg, 4%). (ES, m/z): [M+H]⁺ 465; ¹H NMR (400 MHz, DMSO-d₆) δ 0.90 (t, J=7.5, 7.5 Hz, 3H), 2.28 (s, 5H), 4.24 (s, 3H), 6.99 (d, J=8.3 Hz, 1H), 7.64 (d, J=8.3 Hz, 1H), 7.90-8.04 (m, 3H), 8.10 (d, J=2.0 Hz, 1H), 8.97 (d, J=5.3 Hz, 1H), 10.07 (s, 1H).

### Example 77—Synthesis of N-(6-Ethyl-1-Methyl-1H-Indazol-7-yl)-5-Methyl-1-(2-(Trifluoromethyl)Pyridin-4-yl)-1H-Pyrazole-4-Sulfonamide (I-30)

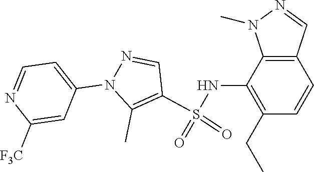 [SMILES: CCc1ccc2cnn(C)c2c1NS(=O)(=O)c1cnn(-c2ccnc(C(F)(F)F)c2)c1C]

The title compound was obtained using general procedure A as a brown solid (59 mg, 21%). (ES, m/z): [M+H]⁺ 465; ¹H NMR (400 MHz, DMSO-d₆) δ 0.87 (t, J=7.5, 7.5 Hz, 3H), 2.24-2.31 (m, 5H), 4.28 (s, 3H), 6.98 (d, J=8.3 Hz, 1H), 7.65 (d, J=8.3 Hz, 1H), 8.00 (s, 1H), 8.22 (dd, J=2.1, 5.6 Hz, 1H), 8.41 (d, J=2.1 Hz, 1H), 8.83 (d, J=5.5 Hz, 1H), 9.25 (s, 1H), 10.22 (s, 1H).

### Example 78—Synthesis of N-(6-Ethyl-1-Methyl-1H-Indazol-7-yl)-1-(2-Methylpyridin-4-yl)-1H-Pyrazole-4-Sulfonamide (I-31)

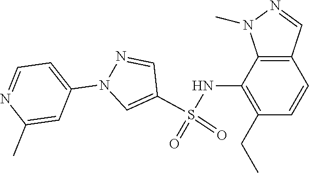 [SMILES: CCc1ccc2cnn(C)c2c1NS(=O)(=O)c1cnn(-c2ccnc(C)c2)c1]

The title compound was obtained using general procedure C as a white solid (20 mg, 13%). (ES, m/z): [M+H]⁺ 397; ¹H NMR (400 MHz, DMSO-d₆) δ 0.86 (t, J=7.6 Hz, 3H), 2.25 (q, J=7.6 Hz, 2H), 2.51 (s, 3H), 4.26 (s, 3H), 6.99 (d, J=8.4 Hz, 1H), 7.64 (d, J=8.4 Hz, 1H), 7.75 (dt, J=5.6, 2.0 Hz, 1H), 7.86 (d, J=1.6 Hz, 1H), 7.99 (s, 1H), 8.07 (s, 1H), 8.54 (d, J=5.6 Hz, 1H), 9.10 (s, 1H), 10.08 (s, 1H).

### Example 79—Synthesis of 1-(2,6-Dimethylpyridin-4-yl)-N-(6-ethyl-1-Methyl-1H-Indazol-7-yl)-1H-Pyrazole-4-Sulfonamide (I-32) ,N

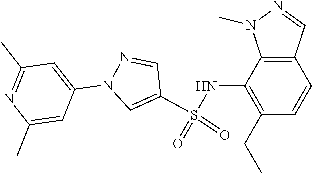 [SMILES: CCc1ccc2cnn(C)c2c1NS(=O)(=O)c1cnn(-c2cc(C)nc(C)c2)c1]

The title compound was obtained using general procedure C as a white solid (20 mg, 13%). (ES, m/z): [M+H]⁺ 411; ¹H NMR (400 MHz, DMSO-d₆) δ 0.86 (t, J=7.6 Hz, 3H), 2.25 (q, J=7.6 Hz, 2H), 2.51 (s, 6H), 4.26 (s, 3H), 6.99 (d, J=8.0 Hz, 1H), 7.55-7.70 (m, 3H), 7.99 (s, 1H), 8.07 (s, 1H), 9.05 (s, 1H), 10.05 (s, 1H).

### Example 80—Synthesis of 1-(2-Cyclopropylpyridin-4-yl)-N-(6-ethyl-1-Methyl-1H-Indazol-7-yl)-1H-Pyrazole-4-Sulfonamide (I-33)

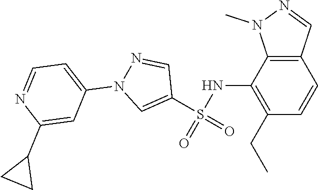 [SMILES: CCc1ccc2cnn(C)c2c1NS(=O)(=O)c1cnn(-c2ccnc(C3CC3)c2)c1]

The title compound was obtained using general procedure D as a white solid (40 mg, 9%). (ES, m/z): [M+H]⁺ 423; ¹H NMR (400 MHz, DMSO-d₆) δ 0.86 (t, J=7.6 Hz, 3H), 1.00-1.10 (m, 4H), 2.25 (q, J=7.6 Hz, 2H), 2.25-2.35 (m, 1H), 4.25 (s, 3H), 6.99 (d, J=8.0 Hz, 1H), 7.60-7.70 (m, 2H), 7.85 (d, J=1.6 Hz, 1H), 7.98 (s, 1H), 8.05 (s, 1H), 8.48 (d, J=5.6 Hz, 1H), 9.11 (s, 1H), 10.07 (s, 1H).

### Example 81—Synthesis of N-(6-Ethyl-1-Methyl-1H-Indazol-7-yl)-1-(4-Methylpyridin-2-yl)-1H-Pyrazole-4-Sulfonamide (I-34)

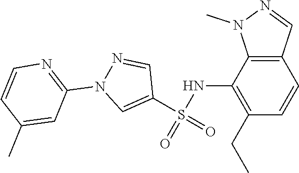 [SMILES: CCc1ccc2cnn(C)c2c1NS(=O)(=O)c1cnn(-c2cc(C)ccn2)c1]

The title compound was obtained using general procedure C as a white solid (48 mg, 18%). (ES, m/z): [M+H]⁺ 397; ¹H NMR (400 MHz, DMSO-d₆) δ 0.84 (t, J=7.6 Hz, 3H), 2.27 (q, J=7.6 Hz, 2H), 2.45 (s, 3H), 4.25 (s, 3H), 6.98 (d, J=8.4 Hz, 1H), 7.32 (dd, J=5.2, 1.2 Hz, 1H), 7.64 (d, J=8.4 Hz, 1H), 7.84 (s, 1H), 8.00 (d, J=1.2 Hz, 2H), 8.36 (d, J=5.2 Hz, 1H), 8.68 (d, J=1.2 Hz, 1H), 10.07 (s, 1H).

### Example 82—Synthesis of N-(6-Methoxy-1-Methyl-1H-Indazol-7-yl)-1-(4-Methylpyridin-2-yl)-1H-Pyrazole-4-Sulfonamide (I-35)

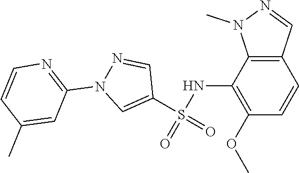 [SMILES: COc1ccc2cnn(C)c2c1NS(=O)(=O)c1cnn(-c2cc(C)ccn2)c1]

The title compound was obtained using general procedure A as a pink solid (32 mg, 13%). (ES, m/z): [M+H]⁺ 398; ¹H NMR (400 MHz, methanol-d₄) δ 2.37 (s, 3H), 3.28 (s, 3H), 4.24 (s, 3H), 6.73 (d, J=8.9 Hz, 1H), 7.132-7.14 (m, 1H), 7.56 (d, J=8.9 Hz, 1H), 7.72 (d, J=0.8 Hz, 1H), 7.74-7.78 (m, 1H), 7.84 (s, 1H), 8.20 (d, J=5.1 Hz, 1H), 8.60 (d, J=0.8 Hz, 1H).

### Example 83—Synthesis of 1-(4-Chloropyridin-2-yl)-N-(6-Ethyl-1-Methyl-1H-indazol-7-yl)-1H-Pyrazole-4-Sulfonamide (I-36)

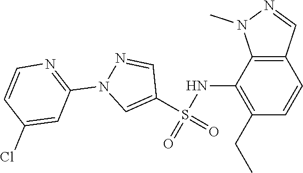 [SMILES: CCc1ccc2cnn(C)c2c1NS(=O)(=O)c1cnn(-c2cc(Cl)ccn2)c1]

The title compound was obtained using general procedure C as a white solid (50 mg, 24%). (ES, m/z): [M+H]⁺ 417; ¹H NMR (400 MHz, DMSO-d₆) δ 0.86 (t, J=7.6 Hz, 3H), 2.26 (q, J=7.6 Hz, 2H), 4.25 (s, 3H), 6.99 (d, J=8.4 Hz, 1H), 7.61-7.69 (m, 2H), 8.00 (s, 1H), 8.03 (s, 1H), 8.07 (s, 1H), 8.52 (d, J=5.6 Hz, 1H), 8.72 (d, J=0.8 Hz, 1H), 10.13 (s, 1H).

### Example 84—Synthesis of 1-(2-Chloropyridin-4-yl)-N-(6-Ethyl-1-Methyl-1H-Indazol-7-yl)-1H-Pyrazole-4-Sulfonamide (I-37)

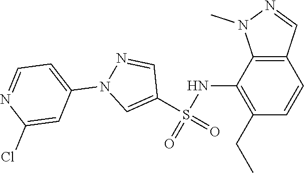 [SMILES: CCc1ccc2cnn(C)c2c1NS(=O)(=O)c1cnn(-c2ccnc(Cl)c2)c1]

The title compound was obtained using general procedure C as a white solid (66 mg, 24%). (ES, m/z): [M+H]⁺ 417; ¹H NMR (400 MHz, DMSO-d₆) δ 0.86 (t, J=7.6 Hz, 3H), 2.25 (q, J=7.6 Hz, 2H), 4.26 (s, 3H), 7.00 (d, J=8.4 Hz, 1H), 7.65 (d, J=8.4 Hz, 1H), 8.01 (d, J=4.4 Hz, 2H), 8.18-8.09 (m, 2H), 8.53 (d, J=5.6 Hz, 1H), 9.24 (s, 1H), 10.16 (s, 1H).

### Example 85—Synthesis of N-(1-Methyl-6-Propyl-1H-Indazol-7-yl)-1-(2-(Trifluoromethyl) Pyridin-4-yl)-1H-Pyrazole-4-Sulfonamide (I-38)

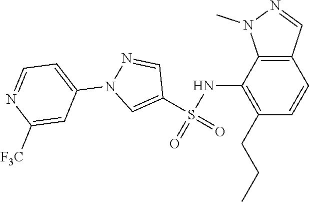 [SMILES: CCCc1ccc2cnn(C)c2c1NS(=O)(=O)c1cnn(-c2ccnc(C(F)(F)F)c2)c1]

The title compound was obtained using general procedure A as a white solid (200 mg, 54%). (ES, m/z): [M+H]⁺ 465; ¹H NMR (400 MHz, DMSO-d₆) δ 0.62 (t, J=7.2 Hz, 3H), 1.25 (s, 2H), 2.21 (s, 2H), 4.27 (s, 3H), 6.99 (d, J=8.4 Hz, 1H), 7.63 (d, J=8.3 Hz, 1H), 8.00 (s, 1H), 8.19 (s, 1H), 8.29 (dd, J=5.5, 2.1 Hz, 1H), 8.49 (d, J=2.1 Hz, 1H), 8.90 (d, J=5.5 Hz, 1H), 9.41 (s, 1H), 10.14 (s, 1H).

### Example 86—Synthesis of N-(5-Ethyl-3-Methyl-1H-Indazol-4-yl)-1-(2-(Trifluoromethyl) Pyridin-4-yl)-1H-Pyrazole-4-Sulfonamide (I-39)

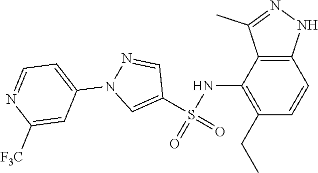 [SMILES: CCc1ccc2[nH]nc(C)c2c1NS(=O)(=O)c1cnn(-c2ccnc(C(F)(F)F)c2)c1]

The title compound was obtained using general procedure A as a white solid (11 mg, 2%). (ES, m/z): [M+H]⁺ 451; ¹H NMR (400 MHz, DMSO-d₆) δ 0.85 (t, J=7.5 Hz, 3H), 2.26 (q, J=7.6 Hz, 2H), 2.57 (s, 3H), 7.17 (d, J=8.6 Hz, 1H), 7.36 (d, J=8.5 Hz, 1H), 8.09 (s, 1H), 8.27 (dd, J=2.1, 5.5 Hz, 1H), 8.47 (d, J=2.1 Hz, 1H), 8.87 (d, J=5.5 Hz, 1H), 9.35 (s, 1H), 9.97 (s, 1H), 12.61 (s, 1H).

### Example 87—Synthesis of N-(5-ethyl-1,3-dimethyl-1H-Indazol-4-yl)-1-(2-(Trifluoromethyl)Pyridin-4-yl)-1H-Pyrazole-4-Sulfonamide (I-40)

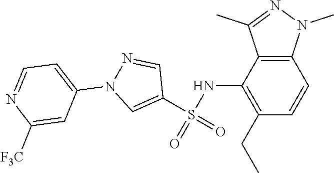 [SMILES: CCc1ccc2c(c(C)nn2C)c1NS(=O)(=O)c1cnn(-c2ccnc(C(F)(F)F)c2)c1]

The title compound was obtained using general procedure A as a white solid (33 mg, 9%) (ES, m/z): [M+H]⁺ 465; ¹H NMR (400 MHz, DMSO-d₆) δ 0.86 (t, J=7.5 Hz, 3H), 2.26 (q, J=7.8 Hz, 2H), 2.55 (s, 3H), 3.91 (s, 3H), 7.22 (d, J=8.7 Hz, 1H), 7.47 (d, J=8.7 Hz, 1H), 8.08 (s, 1H), 8.26 (dd, J=5.6, 2.1 Hz, 1H), 8.46 (d, J=2.1 Hz, 1H), 8.87 (d, J=5.5 Hz, 1H), 9.33 (s, 1H), 9.98 (s, 1H).

### Example 88—Synthesis of 1-(2-(Dimethylamino)Pyridin-4-yl)-N-(6-ethyl-1-Methyl-1H-Indazol-7-yl)-1H-Pyrazole-4-Sulfonamide (I-41)

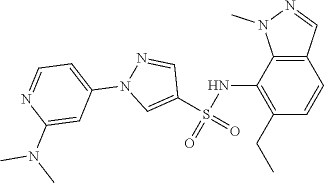 [SMILES: CCc1ccc2cnn(C)c2c1NS(=O)(=O)c1cnn(-c2ccnc(N(C)C)c2)c1]

The title compound was obtained using genera procedure D as a white solid (17 mg, 12%). (ES, m/z): [M+H]⁺ 426; ¹H NMR (400 MHz, DMSO-d₆) δ 0.85 (t, J=7.6 Hz, 3H), 2.25 (q, J=7.6 Hz, 2H), 3.07 (s, 6H), 4.25 (s, 3H), 6.99 (d, J=8.4 Hz, 1H), 7.04 (d, J=1.6 Hz, 1H), 7.11 (dd, J=5.6, 1.2 Hz, 1H), 7.63 (d, J=8.4 Hz, 1H), 8.00 (d, J=5.6 Hz, 2H), 8.16 (d, J=5.6 Hz, 1H), 9.12 (s, 1H), 10.01 (s, 1H).

### Example 89—Synthesis of N-(6-Ethyl-1-Methyl-1H-Indazol-7-yl)-1-(5-Methoxypyridin-2-yl)-1H-Pyrazole-4-Sulfonamide (I-42)

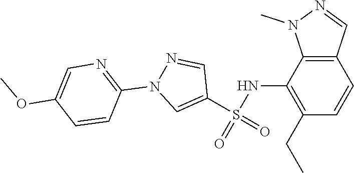 [SMILES: CCc1ccc2cnn(C)c2c1NS(=O)(=O)c1cnn(-c2ccc(OC)cn2)c1]

The title compound was obtained using general procedure D as a white solid (76 mg, 28%). (ES, m/z): [M+H]⁺ 413; ¹H NMR (400 MHz, DMSO-d₆) δ 0.85 (t, J=7.6 Hz, 3H), 2.25 (q, J=7.6 Hz, 2H), 3.90 (s, 3H), 4.25 (s, 3H), 6.99 (d, J=8.4 Hz, 1H), 7.71-7.61 (m, 2H), 7.92 (d, J=8.4 Hz, 1H), 8.02-7.95 (m, 2H), 8.22 (d, J=2.8 Hz, 1H), 8.59 (s, 1H), 10.04 (s, 1H).

### Example 90—Synthesis of N-(6-Ethyl-1-Methyl-1H-Indazol-7-yl)-1-(4-(Trifluoromethoxy) Pyridin-2-yl)-1H-Pyrazole-4-Sulfonamide (I-43)

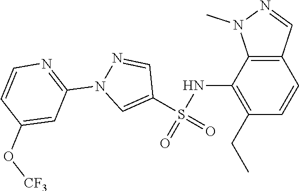 [SMILES: CCc1ccc2cnn(C)c2c1NS(=O)(=O)c1cnn(-c2cc(OC(F)(F)F)ccn2)c1]

The title compound was obtained using general procedure D as a white solid (20 mg, 8%). (ES, m/z): [M+H]⁺ 467; ¹H NMR (400 MHz, DMSO-d₆) δ 0.98 (t, J=7.5 Hz, 3H), 2.25-2.35 (m, 2H), 4.23 (s, 3H), 6.40-6.50 (m, 2H), 7.02 (d, J=8.4 Hz, 1H), 7.63 (d, J=8.4 Hz, 1H), 8.00 (d, J=14.8 Hz, 2H), 8.32 (d, J=8.2 Hz, 1H), 8.72 (s, 1H), 10.25 (s, 1H).

### Example 91—Synthesis of N-(6-Ethyl-1-Methyl-1H-Indazol-7-yl)-1-(4-Methoxypyridin-2-yl)-1H-Pyrazole-4-Sulfonamide (I-44)

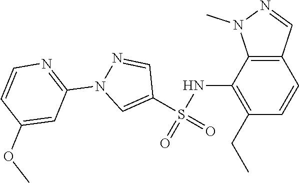 [SMILES: CCc1ccc2cnn(C)c2c1NS(=O)(=O)c1cnn(-c2cc(OC)ccn2)c1]

The title compound was obtained using general procedure D as a white solid (2 mg, 8%). (ES, m/z): [M+H]⁺ 413; ¹H NMR (400 MHz, DMSO-d₆) δ 0.97 (t, J=7.6 Hz, 3H), 2.15-2.22 (m, 2H), 3.37 (s, 3H), 4.18 (s, 3H), 6.84 (dd, J=5.6, 2.0 Hz, 1H), 7.05 (d, J=8.4 Hz, 1H), 7.36 (d, J=2.2 Hz, 1H), 7.71 (d, J=8.4 Hz, 1H), 8.03 (s, 1H), 8.17-8.26 (m, 2H), 8.92 (s, 1H).

### Example 92—Synthesis of N-(3-Methyl-1H-Indazol-4-yl)-1-(5-(Trifluoromethyl)Pyridin-2-yl)-1H-Pyrazole-4-Sulfonamide (I-45)

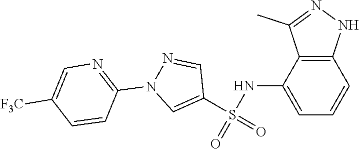 [SMILES: Cc1n[nH]c2cccc(NS(=O)(=O)c3cnn(-c4ccc(C(F)(F)F)cn4)c3)c12]

The title compound was obtained using general procedure A as an off-white solid (149 mg, 34%). (ES, m/z): [M+1]⁺ 423; ¹H NMR (400 MHz, DMSO-d₆) δ 2.61 (s, 3H), 6.57 (d, J=7.3 Hz, 1H), 7.19 (d, J=7.8 Hz, 1H), 7.35 (d, J=8.3 Hz, 1H), 8.00-8.31 (m, 2H), 8.47 (dd, J=2.4, 8.7 Hz, 1H), 8.80-9.31 (m, 2H), 9.98 (s, 1H), 12.74 (s, 1H).

### Example 93—Synthesis of N-(1-Methyl-1H-Indazol-7-yl)-1-(4-(Trifluoromethyl)Pyridin-2-yl)-1H-Pyrazole-4-Sulfonamide (I-46)

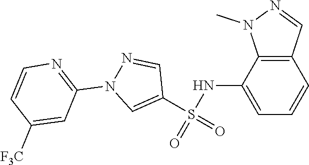 [SMILES: Cn1ncc2cccc(NS(=O)(=O)c3cnn(-c4cc(C(F)(F)F)ccn4)c3)c21]

The title compound was obtained using general procedure A as a brown solid (192 mg, 71%). (ES, m/z): [M+H]⁺ 423; ¹H NMR (400 MHz, DMSO-d₆) δ 4.29 (s, 3H), 6.73 (dd, J=1.0, 7.3 Hz, 1H), 6.98 (t, J=7.7 Hz, 1H), 7.72 (dd, J=1.0, 8.1 Hz, 1H), 7.89 (dd, J=1.6, 5.2 Hz, 1H), 8.04-8.16 (m, 2H), 8.21 (d, J=1.5 Hz, 1H), 8.63-9.26 (m, 2H), 10.10 (s, 1H).

### Example 94—Synthesis of N-(6-Chloro-2-Methyl-2H-Indazol-7-yl)-1-(2-(Trifluoromethyl) Pyridin-4-yl)-1H-Pyrazole-4-Sulfonamide (I-47)

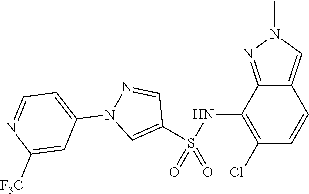 [SMILES: Cn1cc2ccc(Cl)c(NS(=O)(=O)c3cnn(-c4ccnc(C(F)(F)F)c4)c3)c2n1]

The title compound was obtained using general procedure B as a white solid (39 mg, 38%). (ES, m/z): [M+1]=457; ¹H NMR (400 MHz, DMSO-d₆) δ 3.96 (s, 3H), 7.10 (d, J=8.2 Hz, 1H), 7.67 (d, J=8.8 Hz, 1H), 8.20 (d, J=15.2 Hz, 1H), 8.28 (dd, J=5.6 Hz, 2.0 Hz, 1H), 8.37 (s, 1H), 8.47 (d, J=2.0 Hz, 1H), 8.89 (d, J=5.6 Hz, 1H), 9.39 (s, 1H), 10.22 (s, 1H).

### Example 95—Synthesis of N-(6-Ethyl-2-Methyl-2H-Indazol-7-yl)-1-(2-(Trifluoromethyl) Pyridin-4-yl)-1H-Pyrazole-4-Sulfonamide (I-48)

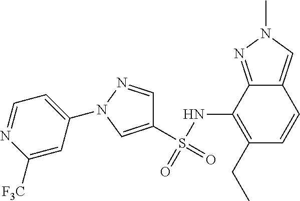 [SMILES: CCc1ccc2cn(C)nc2c1NS(=O)(=O)c1cnn(-c2ccnc(C(F)(F)F)c2)c1]

The title compound was obtained using general procedure A as a white solid (16 mg, 20%). (ES, m/z): [M+1]=451; ¹H NMR (400 MHz, DMSO-d₆) δ 1.18 (t, J=7.6 Hz, 3H), 2.83 (q, J=7.6 Hz, 1.2 Hz, 2H), 3.82 (s, 3H), 6.99 (d, J=8.0 Hz, 1H), 7.75 (d, J=8.4 Hz, 1H), 8.11 (s, 1H), 8.18 (s, 1H), 8.24 (dd, J=5.2 Hz, 1.6 Hz, 2H), 8.42 (d, J=2.0 Hz, 1H), 8.87 (d, J=5.6 Hz, 1H), 9.27 (s, 1H), 9.76 (s, 1H).

### Example 96—Synthesis of N-(5-Bromo-1-Methyl-1H-Indazol-7-yl)-1-(2-(Trifluoromethyl) Pyridin-4-yl)-1H-Pyrazole-4-Sulfonamide

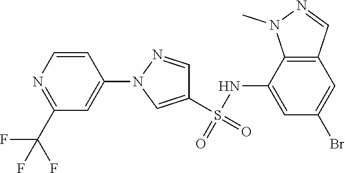 [SMILES: Cn1ncc2cc(Br)cc(NS(=O)(=O)c3cnn(-c4ccnc(C(F)(F)F)c4)c3)c21]

The title compound was obtained using general procedure A as a yellow solid (380 mg, 57%). (ES, m/z): [M+1]=501.

### Example 97—Synthesis of N-(1,5-Dimethyl-1H-Indazol-7-yl)-1-(2-(Trifluoromethyl) Pyridin-4-yl)-1H-Pyrazole-4-Sulfonamide (I-49)

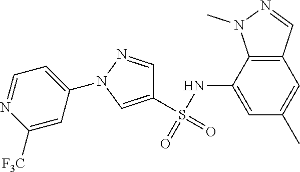 [SMILES: Cc1cc(NS(=O)(=O)c2cnn(-c3ccnc(C(F)(F)F)c3)c2)c2c(cnn2C)c1]

To a stirred solution of N-(5-bromo-1-Methyl-1H-Indazol-7-yl)-1-(2-(trifluoromethyl) pyridin-4-yl)-1H-pyrazole-4-sulfonamide (200 mg, 0.4 mmol, 1 eq.) and trimethyl-1,3,5,2,4,6-trioxatriborinane (150 mg, 1.2 mmol, 3 eq.) in dioxane (5 mL) were added Pd(dppf)Cl₂—CH₂C12 (33 mg, 0.04 mmol, 0.1 eq.) and Cs₂CO₃ (260 mg, 0.8 mmol, 2 eq.) at rt under nitrogen. The solution was stirred at 90° C. for 5 h, and then was filtered, the solids were washed with EtOAc (3×50 mL), and the filtrate was concentrated under reduced pressure. The residue was purified by Prep-TLC (PE/EA=3/1) to afford the title compound (50 mg, 29%) as an off-white solid. (ES, m z): [M+H]⁺ 437; ¹H NMR (400 MHz, DMSO-d₆) δ 2.20 (s, 3H), 4.23 (s, 3H), 6.56 (d, J=1.2 Hz, 1H), 7.47 (t, J=1.2 Hz, 1H), 7.97 (s, 1H), 8.17 (s, 1H), 8.28 (dd, J=5.6, 2.2 Hz, 1H), 8.48 (d, J=2.1 Hz, 1H), 8.89 (d, J=5.5 Hz, 1H), 9.38 (s, 1H), 10.06 (s, 1H).

### Example 98—Synthesis of N-(6-Methoxy-2-Methyl-2H-Indazol-7-yl)-1-(2-(Trifluoromethyl)Pyridin-4-yl)-1H-Pyrazole-4-Sulfonamide (I-50)

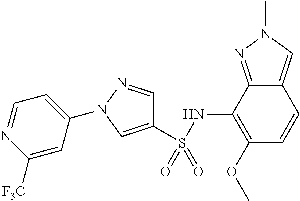 [SMILES: COc1ccc2cn(C)nc2c1NS(=O)(=O)c1cnn(-c2ccnc(C(F)(F)F)c2)c1]

The title compound was obtained using general procedure A as an off-white solid (6 mg, 3%). (ES, m/z): [M+H]⁺ 453; ¹H NMR (400 MHz, DMSO-d₆) δ 3.63 (s, 3H), 4.01 (s, 3H), 6.97 (d, J=9.2 Hz, 1H), 7.63 (d, J=9.2 Hz, 1H), 8.19 (s, 1H), 8.28 (t, J=6.0 Hz, 2H), 8.47 (d, J=2.0 Hz, 1H), 8.80 (d, J=5.6 Hz, 1H), 9.36 (s, 1H).

### Example 99—Synthesis of N-(3-Methyl-1H-Indazol-4-yl)-1-(2-(Trifluoromethyl)Pyridin-4-yl)-1H-Pyrazole-4-Sulfonamide (I-51)

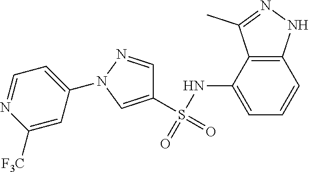 [SMILES: Cc1n[nH]c2cccc(NS(=O)(=O)c3cnn(-c4ccnc(C(F)(F)F)c4)c3)c12]

The title compound was obtained using general procedure A as a white solid (59 mg, 13%). (ES, m/z): [M+1]⁺423; ¹H NMR (400 MHz, DMSO-d₆) δ 2.61 (s, 3H), 6.57 (d, J=7.3 Hz, 1H), 7.19 (d, J=7.8 Hz, 1H), 7.35 (d, J=8.3 Hz, 1H), 8.00-8.31 (m, 2H), 8.47 (dd, J=8.7, 2.4 Hz, 1H), 8.80-9.31 (m, 2H), 9.98 (s, 1H), 12.74 (s, 1H).

### Example 100—Synthesis of N-(6-Methoxy-1-Methyl-1H-Benzo[D[1,2,3]Triazol-7-yl)-1-(2-(Trifluoromethyl)Pyridin-4-yl)-1H-Pyrazole-4-Sulfonamide (I-52)

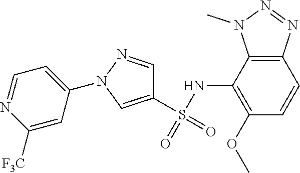 [SMILES: COc1ccc2nnn(C)c2c1NS(=O)(=O)c1cnn(-c2ccnc(C(F)(F)F)c2)c1]

The title compound was obtained using general procedure B as a white solid (48 mg, 21%). (ES, m/z): [M+H]⁺ 453; ¹H NMR (400 MHz, DMSO-d₆) δ 3.38 (s, 3H), 4.45 (s, 3H), 7.16 (d, J=9.2 Hz, 1H), 7.98 (d, J=9.2 Hz, 1H), 8.16 (s, 1H), 8.27 (dd, J=2.0, 5.2 Hz, 2H), 8.46 (d, J=2.0 Hz, 1H), 8.88 (d, J=5.6 Hz, 1H), 9.31 (s, 1H), 10.11 (s, 1H).

### Example 101—Synthesis of N-(6-Cyclopropyl-1-Methyl-1H-Indazol-7-yl)-1-(6-(Trifluoromethyl)Pyrimidin-4-yl)-1H-Pyrazole-4-Sulfonamide (I-53)

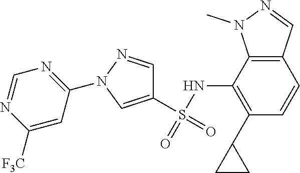 [SMILES: Cn1ncc2ccc(C3CC3)c(NS(=O)(=O)c3cnn(-c4cc(C(F)(F)F)ncn4)c3)c21]

The title compound was obtained using general procedure C as a white solid (42 mg, 26%). (ES, m/z): [M+H]⁺ 464; ¹H NMR (400 MHz, DMSO-d₆) δ 0.40-0.60 (m, 4H), 1.80-1.90 (m, 1H), 4.29 (s, 3H), 6.36 (d, J=8.4 Hz, 1H), 7.57 (d, J=8.4 Hz, 1H), 7.97 (s, 1H), 8.19 (s, 1H), 8.33 (s, 1H), 8.81 (s, 1H), 9.39 (s, 1H), 10.35 (s, 1H).

### Example 102—Synthesis of 1-(4-Chloropyridin-2-yl)-N-(6-Methoxy-1-Methyl-1H-Benzo[D][1,2,3]Triazol-7-yl)-1H-Pyrazole-4-Sulfonamide (I-54)

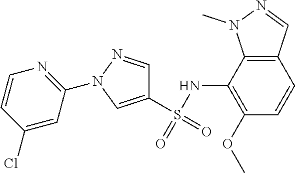 [SMILES: COc1ccc2cnn(C)c2c1NS(=O)(=O)c1cnn(-c2cc(Cl)ccn2)c1]

The title compound was obtained using general procedure A as an off-white solid (2 mg, 3%). (ES, m/z): [M+H]⁺ 419; ¹H NMR (400 MHz, DMSO-d₆) δ 3.39 (s, 3H), 4.25 (3, 3H), 6.87 (dd, J=1.7, 8.9 Hz, 1H), 7.62-7.67 (m, 2H), 7.98 (d, J=1.7 Hz, 1H), 8.03 (d, J=1.8 Hz, 2H), 8.52 (dd, J=1.6, 5.4 Hz, 1H), 8.70 (d, J=1.6 Hz, 1H), 9.76 (s, 1H).

### Example 103—Synthesis of N-(6-Cyclopropoxy-1-Methyl-1H-Indazol-7-yl)-1-(2-(Trifluoromethyl)Pyridin-4-yl)-1H-Pyrazole-4-Sulfonamide (I-55)

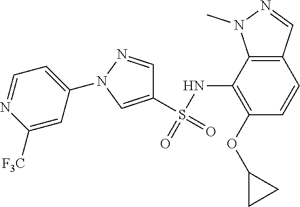 [SMILES: Cn1ncc2ccc(OC3CC3)c(NS(=O)(=O)c3cnn(-c4ccnc(C(F)(F)F)c4)c3)c21]

The title compound was obtained using general procedure A as a white solid (22 mg, 10%). (ES, m/z): [M+H]⁺ 479; ¹H NMR (400 MHz, DMSO-d₆) δ 0.12 (m, 2H), 0.45 (m, 2H), 3.49 (tt, J=6.0, 2.9 Hz, 1H), 4.25 (s, 3H), 7.16 (d, J=8.8 Hz, 1H), 7.70 (d, J=8.8 Hz, 1H), 7.99 (s, 1H), 8.11 (s, 1H), 8.30 (dd, J=5.5, 2.1 Hz, 1H), 8.50 (d, J=2.1 Hz, 1H), 8.89 (d, J=5.5 Hz, 1H), 9.35 (s, 1H), 9.80 (s, 1H).

### Example 104—Synthesis of 1-(4-Cyclopropylpyridin-2-yl)-N-(6-Ethyl-1-Methyl-1H-Indazol-7-yl)-1H-Pyrazole-4-Sulfonamide (I-56)

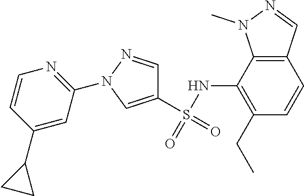 [SMILES: CCc1ccc2cnn(C)c2c1NS(=O)(=O)c1cnn(-c2cc(C3CC3)ccn2)c1]

The title compound was obtained using general procedure A as an off-white solid (54 mg, 20%). (ES, m/z): [M+H]⁺ 423; ¹H NMR (400 MHz, CDCl₃) δ 0.88-0.92 (m, 2H), 0.99 (t, J=7.6 Hz, 3H), 1.12-1.22 (m, 2H), 1.92-2.03 (m, 1H), 2.27 (q, J=7.6 Hz, 2H), 4.40 (s, 3H), 6.44 (s, 1H), 6.91-6.98 (m, 2H), 7.63 (dd, J=3.3, 4.9 Hz, 2H), 7.69 (d, J=0.8 Hz, 1H), 7.95 (s, 1H), 8.21 (d, J=5.3 Hz, 1H), 8.77 (d, J=0.8 Hz, 1H).

### Example 105—Synthesis of 1-(4-Cyclopropylpyridin-2-yl)-N-(6-Methoxy-1-Methyl-1H-Indazol-7-yl)-1H-Pyrazole-4-Sulfonamide (I-57)

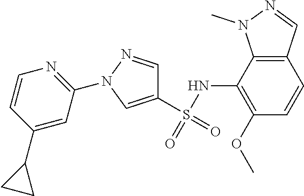 [SMILES: COc1ccc2cnn(C)c2c1NS(=O)(=O)c1cnn(-c2cc(C3CC3)ccn2)c1]

The title compound was obtained using general procedure A as an off-white solid (8 mg, 3%). (ES, m/z): [M+H]⁺ 425; ¹H NMR (400 MHz, CDCl₃) δ 0.89-0.92 (m, 2H), 1.10-1.23 (m, 2H), 1.91-2.03 (m, 1H), 3.40 (s, 3H), 4.40 (s, 3H), 6.47 (s, 1H), 6.66 (d, J=8.8 Hz, 1H), 6.93 (dd, J=1.6, 5.3 Hz, 1H), 7.56-7.63 (m, 2H), 7.68 (s, 1H), 7.93 (s, 1H), 8.21 (d, J=5.2 Hz, 1H), 8.71 (s, 1H).

### Example 106—Synthesis of N-(5-Methoxy-1-Methyl-1H-Indazol-7-yl)-1-(2-(Trifluoromethyl)Pyridin-4-yl)-1H-Pyrazole-4-Sulfonamide (I-58)

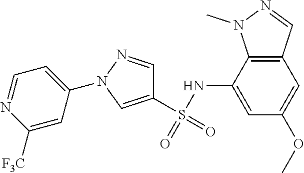 [SMILES: COc1cc(NS(=O)(=O)c2cnn(-c3ccnc(C(F)(F)F)c3)c2)c2c(cnn2C)c1]

The title compound was obtained using general procedure B as an off-white solid (60 mg, 21%). (ES, m/z): [M+H]⁺ 453; ¹H NMR (400 MHz, DMSO-d₆) δ 3.64 (s, 3H), 4.23 (s, 3H), 6.41 (d, J=2.4 Hz, 1H), 7.06 (s, 1H), 7.91 (s, 1H), 8.16 (s, 1H), 8.27 (dd, J=5.6, 2.0 Hz, 1H), 8.47 (d, J=2.0 Hz, 1H), 8.88 (d, J=5.6 Hz, 1H), 9.39 (s, 1H), 10.16 (s, 1H).

### Example 107—Synthesis of N-(1,6-Dimethyl-1H-Indazol-7-yl)-1-(2-(Trifluoromethyl)Pyridin-4-yl)-1H-Pyrazole-4-Sulfonamide (I-59)

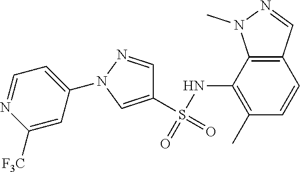 [SMILES: Cc1ccc2cnn(C)c2c1NS(=O)(=O)c1cnn(-c2ccnc(C(F)(F)F)c2)c1]

The title compound was obtained using general procedure A as a white solid (23 mg, 17%). (ES, m/z): [M+H]⁺=437; ¹H NMR (400 MHz, DMSO-d₆) δ 1.89 (s, 3H), 4.25 (s, 3H), 6.91 (d, J=8.4 Hz, 1H), 7.58 (d, J=8 Hz, 1H), 7.99 (s, 1H), 8.13 (s, 1H), 8.28 (dd, J=5.2 Hz, 1 Hz, 1H), 8.48 (d, J=2 Hz, 1H), 8.88 (d, J=5.6 Hz, 1H), 9.39 (s, 1H).

### Example 108—Synthesis of N-(1,6-Dimethyl-1H-Indazol-7-yl)-1-(4-(Trifluoromethyl) Pyridin-2-yl)-1H-Pyrazole-4-Sulfonamide (I-60)

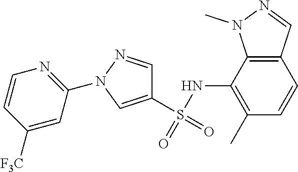 [SMILES: Cc1ccc2cnn(C)c2c1NS(=O)(=O)c1cnn(-c2cc(C(F)(F)F)ccn2)c1]

The title compound was obtained using general procedure A as a white solid (62 mg, 36%). (ES, m/z): [M+H]⁺ 437; ¹H NMR (400 MHz, DMSO-d₆) δ 1.89 (s, 3H), 4.26 (s, 3H), 6.91 (d, J=8.0 Hz, 1H), 7.60 (d, J=8.0 Hz, 1H), 7.89 (d, J=5.3 Hz, 1H), 8.01 (s, 1H), 8.09 (s, 1H), 8.20 (s, 1H), 8.82 (d, J=4.8 Hz, 1H), 10.12 (s, 1H).

### Example 109—Synthesis of N-(6-Ethyl-1-Methyl-1H-Indazol-7-yl)-1-(2-(Trifluoromethoxy) Pyridin-4-yl)-1H-Pyrazole-4-Sulfonamide (I-61)

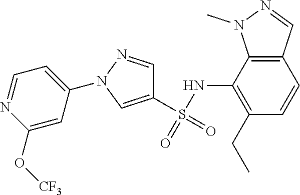 [SMILES: CCc1ccc2cnn(C)c2c1NS(=O)(=O)c1cnn(-c2ccnc(OC(F)(F)F)c2)c1]

The title compound was obtained using general procedure D as a white solid (2 mg, 6%). (ES, m/z): [M+H]⁺ 467; ¹H NMR (400 MHz, DMSO-d₆) δ 0.85 (t, J=7.5 Hz, 3H), 2.20-2.30 (m, 2H), 4.25 (s, 3H), 6.98 (d, J=8.3 Hz, 1H), 7.62 (d, J=8.4 Hz, 1H), 7.85 (d, J=1.9 Hz, 1H), 7.96-8.04 (m, 2H), 8.11 (s, 1H), 8.48 (d, J=5.7 Hz, 1H), 9.21 (s, 1H).

### Example 110—Synthesis of N-(5-Ethyl-3-Methyl-1H-Indazol-4-yl)-N-Methyl-1-(2-(Trifluoromethyl)Pyridin-4-yl)-1H-Pyrazole-4-Sulfonamide (I-62)

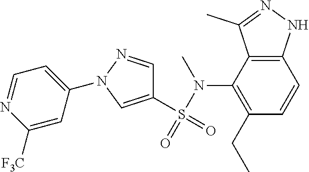 [SMILES: CCc1ccc2[nH]nc(C)c2c1N(C)S(=O)(=O)c1cnn(-c2ccnc(C(F)(F)F)c2)c1]

The title compound was obtained using general procedure E as a white solid (15 mg, 20%). (ES, m/z): [M+H]⁺ 465; ¹H NMR (400 MHz, methanol-d₄) δ 1.06 (t, J=7.5 Hz, 3H), 2.27-2.30 (m, 2H), 2.62 (s, 3H), 3.42 (s, 3H), 7.31 (d, J=8.7 Hz, 1H), 7.46 (d, J=8.6 Hz, 1H), 8.12 (s, 1H), 8.18 (dd, J=5.5, 2.2 Hz, 1H), 8.40 (d, J=2.1 Hz, 1H), 8.82 (d, J=5.5 Hz, 1H), 9.19 (s, 1H).

### Example 111—Synthesis of N-(3-Methyl-1H-Indazol-4-yl)-1-(4-Methylpyridin-2-yl)-1H-Pyrazole-4-Sulfonamide (I-63)

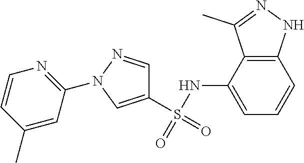 [SMILES: Cc1ccnc(-n2cc(S(=O)(=O)Nc3cccc4[nH]nc(C)c34)cn2)c1]

The title compound was obtained using general procedure A as a white solid (2 mg, 9%). (ES, m/z): [M+H]⁺ 426; ¹H NMR (400 MHz, DMSO-d₆) δ 2.43 (s, 3H), 2.59 (s, 3H), 6.57 (s, 1H), 7.14 (s, 1H), 7.29 (s, 2H), 7.81 (s, 1H), 7.96 (s, 1H), 8.34 (s, 1H), 8.73 (s, 1H), 9.88 (s, 1H), 12.65 (s, 1H).

### Example 112—Synthesis of N-Methyl-N-(3-Methyl-1H-Indazol-4-yl)-1-(2-(Trifluoromethyl)Pyridin-4-yl)-1H-Pyrazole-4-Sulfonamide (I-64)

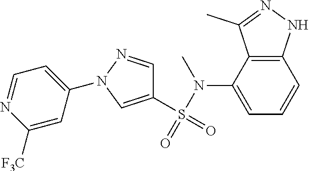 [SMILES: Cc1n[nH]c2cccc(N(C)S(=O)(=O)c3cnn(-c4ccnc(C(F)(F)F)c4)c3)c12]

The title compound was obtained using general procedure E as a white solid (14 mg, 47%). (ES, m/z): [M+H]⁺ 437; ¹H NMR (400 MHz, DMSO-d₆) δ 2.65 (s, 3H), 3.32 (s, 3H), 6.57 (d, J=7.6 Hz, 1H), 7.22 (t, J=7.6 Hz, 1H), 7.45 (d, J=8.4 Hz, 1H), 8.11 (s, 1H), 8.33 (dd, J=5.6 Hz, 2 Hz, 1H), 8.55 (d, J=2 Hz, 1H), 8.92 (d, J=5.2 Hz, 1H), 9.59 (s, 1H).

### Example 113—Synthesis of N-Methyl-N-(3-Methyl-1H-Indazol-4-yl)-1-(4-(Trifluoromethyl)Pyridin-2-yl)-1H-Pyrazole-4-Sulfonamide (I-65)

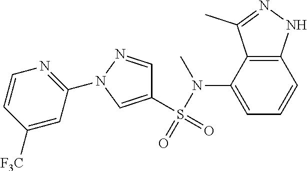 [SMILES: Cc1n[nH]c2cccc(N(C)S(=O)(=O)c3cnn(-c4cc(C(F)(F)F)ccn4)c3)c12]

The title compound was obtained using general procedure E as a white solid (15 mg, 38%). (ES, m/z): [M+H]⁺ 437; ¹H NMR (400 MHz, DMSO-d₆) δ 2.66 (s, 3H), 3.27 (s, 3H), 6.58 (d, J=7.2 Hz, 1H), 7.23 (t, J=7.6 Hz, 1H), 7.64 (d, J=8.0 Hz, 1H), 7.91 (d, J=5.2 Hz, 1H), 8.13 (s, 1H), 8.25 (s, 1H), 8.86 (d, J=5.2 Hz, 1H), 9.02 (s, 1H), 12.84 (s, 1H).

### Example 114—Synthesis of N-Methyl-N-(3-Methyl-1H-Indazol-4-yl)-1-(4-Methylpyridin-2-yl)-1H-Pyrazole-4-Sulfonamide (I-66)

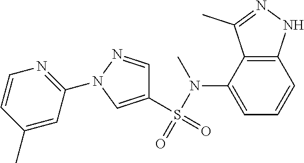 [SMILES: Cc1ccnc(-n2cc(S(=O)(=O)N(C)c3cccc4[nH]nc(C)c34)cn2)c1]

The title compound was obtained using general procedure E as an off-white solid (12 mg, 31%). (ES, m/z): [M+H]⁺ 383; ¹H NMR (400 MHz, CDCl₃) δ 2.48 (s, 3H), 2.85 (s, 3H), 3.31 (s, 3H), 6.57 (dd, J=0.7, 7.4 Hz, 1H), 7.11-7.14 (m, 1H), 7.25 (dd, J=7.3, 8.4 Hz, 1H), 7.42 (dd, J=0.8, 8.4 Hz, 1H), 7.81 (d, J=0.8 Hz, 1H), 7.87 (dt, J=0.8, 1.5 Hz, 1H), 8.31 (dd, J=0.8, 5.0 Hz, 1H), 8.94 (d, J=0.8 Hz, 1H).

### Example 115—Synthesis of N-(3,5-Dimethyl-1H-Indazol-4-yl)-N-Methyl-1-(4-(Trifluoromethyl)Pyridin-2-yl)-1H-Pyrazole-4-Sulfonamide (I-67)

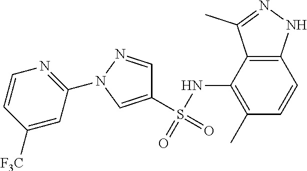 [SMILES: Cc1ccc2[nH]nc(C)c2c1NS(=O)(=O)c1cnn(-c2cc(C(F)(F)F)ccn2)c1]

The title compound was obtained using general procedure A as a white solid (31 mg, 26%). (ES, m/z): [M+H]⁺ 437; ¹H NMR (400 MHz, DMSO-d₆) δ 1.89 (s, 3H), 2.57 (s, 3H), 7.10 (d, J=8.5 Hz, 1H), 7.31 (d, J=8.5 Hz, 1H), 7.88 (dd, J=5.3, 1.6 Hz, 1H), 8.03 (d, J=0.8 Hz, 1H), 8.19 (d, J=1.6 Hz, 1H), 8.76 (d, J=0.8 Hz, 1H), 8.81 (d, J=5.1 Hz, 1H), 9.91 (s, 1H), 12.61 (s, 1H).

### Example 116—Synthesis of N-(3-Methyl-1H-Indazol-4-yl)-1-(4-(Trifluoromethyl)Pyridin-2-yl)-1H-Pyrazole-4-Sulfonamide (I-73)

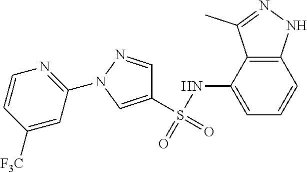 [SMILES: Cc1n[nH]c2cccc(NS(=O)(=O)c3cnn(-c4cc(C(F)(F)F)ccn4)c3)c12]

The title compound was obtained using general procedure A as a white solid (22 mg, 45%). (ES, m/z): [M+1]=423; ¹H NMR (400 MHz, DMSO-d₆) δ 2.60 (s, 3H), 6.56 (d, J=7.2 Hz, 1H), 7.15 (t, J=7.6 Hz, 1H), 7.30 (d, J=8.0 Hz, 1H), 7.87 (d, J=4.4 Hz, 1H), 8.08 (s, 1H), 8.17 (s, 1H), 8.81 (d, J=5.2 Hz, 1H), 8.85 (s, 1H), 9.91 (s, 1H), 12.71 (s, 1H).

### Example 117—Synthesis of N-(3,5-Dimethyl-1H-Indazol-4-yl)-1-(2-(Trifluoromethyl)Pyridin-4-yl)-1H-Pyrazole-4-Sulfonamide (I-74)

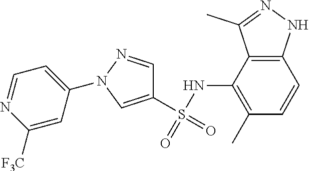 [SMILES: Cc1ccc2[nH]nc(C)c2c1NS(=O)(=O)c1cnn(-c2ccnc(C(F)(F)F)c2)c1]

The title compound was obtained using general procedure A as a white solid (29 mg, 52%). (ES, m/z): [M+H]⁺ 437; ¹H NMR (400 MHz, DMSO-d₆) δ 1.89 (s, 3H), 2.57 (s, 3H), 7.10 (d, J=8.5 Hz, 1H), 7.30 (d, J=8.4 Hz, 1H), 8.07 (s, 1H), 8.27 (dd, J=5.5, 2.1 Hz, 1H), 8.47 (d, J=2.2 Hz, 1H), 8.87 (d, J=5.5 Hz, 1H), 9.35 (s, 1H), 9.91 (s, 1H), 12.59 (s, 1H).

### Example 118—Synthesis of 1-(2-Cyclopropylpyridin-4-yl)-N-(1-Methyl-1H-Indazol-7-yl)-1H-Pyrazole-4-Sulfonamide (I-75)

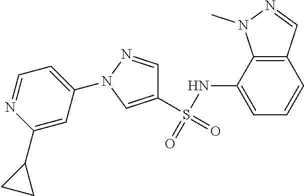 [SMILES: Cn1ncc2cccc(NS(=O)(=O)c3cnn(-c4ccnc(C5CC5)c4)c3)c21]

The title compound was obtained using general procedure A as an off-white solid (14 mg, 10%). (ES, m/z): [M+H]⁺ 395; ¹H NMR (400 MHz, DMSO-d₆) δ 0.99 (dd, J=5.0, 7.3 Hz, 4H), 2.13-2.18 (m, 1H), 4.29 (s, 3H), 6.71 (d, J=7.3 Hz, 1H), 6.98 (t, J=7.7 Hz, 1H), 7.65-7.72 (m, 2H), 7.86 (d, J=2.1 Hz, 1H), 8.08 (d, J=1.6 Hz, 2H), 8.48 (d, J=5.5 Hz, 1H), 9.13 (s, 1H), 10.06 (s, 1H).

### Example 119—1-(4-Cyclopropylpyridin-2-yl)-N-(1-Methylindazol-7-yl)Pyrazole-4-Sulfonamide (I-76)

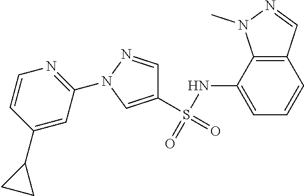 [SMILES: Cn1ncc2cccc(NS(=O)(=O)c3cnn(-c4cc(C5CC5)ccn4)c3)c21]

The title compound was obtained using general procedure C as a white solid (9 mg, 7%). (ES, m/z): [M+H]⁺ 395; ¹H NMR (400 MHz, DMSO-d₆) δ 0.87-0.95 (m, 2H), 1.11-1.20 (m, 2H), 2.13 (tt, J=8.5, 4.9 Hz, 1H), 4.29 (s, 3H), 6.67-6.73 (m, 1H), 6.97 (t, J=7.7 Hz, 1H), 7.16 (dd, J=5.2, 1.6 Hz, 1H), 7.66-7.72 (m, 2H), 8.00 (s, 1H), 8.09 (s, 1H), 8.31 (d, J=5.2 Hz, 1H), 8.77 (s, 1H), 10.04 (s, 1H).

### Example 120—Synthesis of 1-(2-Cyclopropylpyridin-4-yl)-N-(6-Methoxy-1-Methyl-1H-Indazol-7-yl)-1H-Pyrazole-4-Sulfonamide (I-77)

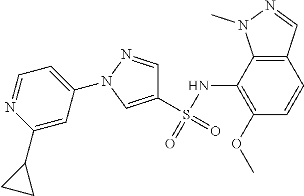 [SMILES: COc1ccc2cnn(C)c2c1NS(=O)(=O)c1cnn(-c2ccnc(C3CC3)c2)c1]

The title compound was obtained using general procedure A as an off-white solid (21 mg, 16%). (ES, m/z): [M+H]⁺ 425; ¹H NMR (400 MHz, DMSO-d₆) δ 0.95-1.03 (m, 4H), 2.13-2.18 (m, 1H), 3.30 (s, 3H), 4.25 (s, 3H), 6.87 (d, J=8.9 Hz, 1H), 7.63-7.71 (m, 2H), 7.87 (d, J=2.1 Hz, 1H), 7.97 (s, 1H), 8.03 (s, 1H), 8.47 (d, J=5.5 Hz, 1H), 9.06 (s, 1H), 9.77 (s, 1H).

### Example 121—Synthesis of N-(5-Ethyl-3-Methyl-1-((2-(Trimethylsilyl)Ethoxy)Methyl)-1H-Indazol-4-yl)-1-(4-Methylpyridin-2-yl)-1H-Pyrazole-4-Sulfonamide

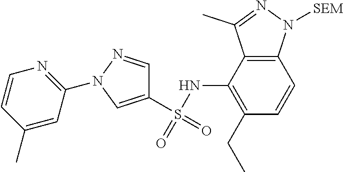 [SMILES: CCc1ccc2c(c(C)nn2COCC[Si](C)(C)C)c1NS(=O)(=O)c1cnn(-c2cc(C)ccn2)c1]

The title compound was obtained using general procedure A as a white solid (58 mg, 10%). (ES, m/z): [M+H]⁺ 527.

### Example 122—Synthesis of N-(5-Ethyl-3-Methyl-1-((2-(Trimethylsilyl)Ethoxy)Methyl)-1H-Indazol-4-yl)-N-Methyl-1-(4-Methylpyridin-2-yl)-1H-Pyrazole-4-Sulfonamide

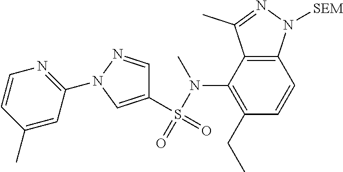 [SMILES: CCc1ccc2c(c(C)nn2COCC[Si](C)(C)C)c1N(C)S(=O)(=O)c1cnn(-c2cc(C)ccn2)c1]

The title compound was obtained using general procedure E as a yellow oil. (80 mg, 91%) (ES, m/z): [M+H]⁺ 541.

### Example 123—Synthesis of N-(5-Ethyl-3-Methyl-1H-Indazol-4-yl)-N-Methyl-1-(4-Methylpyridin-2-yl)Pyrazole-4-Sulfonamide (I-78)

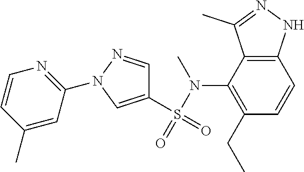 [SMILES: CCc1ccc2[nH]nc(C)c2c1N(C)S(=O)(=O)c1cnn(-c2cc(C)ccn2)c1]

To a stirred solution of N-(5-ethyl-3-methyl-1-{[2-(trimethylsilyl)ethoxy]methyl}indazol-4-yl)-N-methyl-1-(4-methylpyridin-2-yl)pyrazole-4-sulfonamide (70 mg, 0.13 mmol, 1 eq.) in DCM (3 mL) was added TFA (3 mL). The resulting mixture was stirred for 2 h at room temperature, and then concentrated under reduced pressure. The residue was then dissolved in NH₃. H₂O (3 mL) and MeCN (3 mL), and the resulting mixture was stirred for an additional 2 h at room temperature. The mixture was then concentrated under reduced pressure, and the residue was diluted with H₂O (20 mL) and extracted with EtOAc (2×20 mL). The combined organic layers were washed with brine (50 mL), dried over Na₂SO₄, and concentrated under reduced pressure. The crude product (70 mg) was purified by Prep-HPLC to afford the tittle compound (13 mg, 25%) as a white solid. (ES, m/z): [M+1]=411; ¹H NMR (400 MHz, CDCl₃) δ 1.09 (t, J=7.5 Hz, 3H), 2.33 (q, J=7.5 Hz, 2H), 2.47 (s, 3H), 2.74 (s, 3H), 3.38 (s, 3H), 7.12 (m, 1H), 7.30 (d, J=8.7 Hz, 2H), 7.47 (d, J=8.6 Hz, 1H), 7.87 (m, 2H), 8.30 (d, J=5.0 Hz, 1H), 8.94 (d, J=0.8 Hz, 1H).

### Example 124—Synthesis of N-(5-Methoxy-3-Methyl-1-((2-(Trimethyl-Silyl)Ethoxy)Methyl)-1H-Indazol-4-yl)-1-(2-(Trifluoromethyl) Pyridin-4-yl)-1H-Pyrazole-4-Sulfonamide

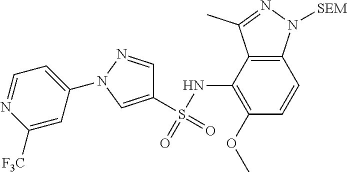 [SMILES: COc1ccc2c(c(C)nn2COCC[Si](C)(C)C)c1NS(=O)(=O)c1cnn(-c2ccnc(C(F)(F)F)c2)c1]

The title compound was obtained using general procedure A as a white solid (62 mg, 12%). (ES, m/z): [M+H]⁺ 583.

### Example 125—Synthesis of N-(5-Methoxy-3-Methyl-1H-Indazol-4-yl)-1-(2-(Trifluoromethyl)Pyridin-4-yl)-1H-Pyrazole-4-Sulfonamide (I-79)

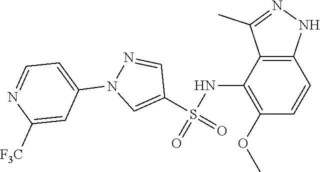 [SMILES: COc1ccc2[nH]nc(C)c2c1NS(=O)(=O)c1cnn(-c2ccnc(C(F)(F)F)c2)c1]

SEM-deprotection was carried out following the same procedure as reported above. The crude product (70 mg) was purified by Prep-HPLC to afford the title compound (14 mg, 18%) as a white solid. (ES, m/z): [M+H]⁺ 453; ¹H NMR (400 MHz, methanol-d₄) δ 2.76 (s, 3H), 3.39 (s, 3H), 7.09 (d, J=9.0 Hz, 1H), 7.38 (d, J=9.0 Hz, 1H), 7.98 (s, 1H), 8.08 (dd, J=5.5, 2.2 Hz, 1H), 8.31 (d, J=2.1 Hz, 1H), 8.78 (d, J=5.5 Hz, 1H), 8.82 (s, 1H).

### Example 126—Synthesis of N-(5-Methoxy-3-Methyl-1-((2-(Trimethylsilyl)Ethoxy)Methyl)-1H-Indazol-4-yl)-N-Methyl-1-(2-(Trifluoromethyl)Pyridin-4-yl)-1H-Pyrazole-4-Sulfonamide

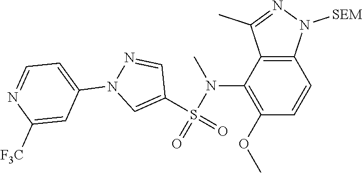 [SMILES: COc1ccc2c(c(C)nn2COCC[Si](C)(C)C)c1N(C)S(=O)(=O)c1cnn(-c2ccnc(C(F)(F)F)c2)c1]

The title compound was obtained using general procedure E as a yellow solid (100 mg, 65%). (ES, m/z): [M+H]⁺ 597.

### Example 127—Synthesis of N-(5-methoxy-3-Methyl-1H-Indazol-4-yl)-N-Methyl-1-(2-(Trifluoromethyl)Pyridin-4-yl)-1H-Pyrazole-4-Sulfonamide (I-80)

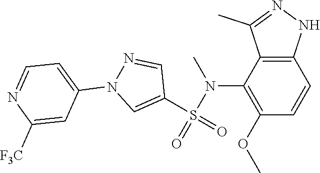 [SMILES: COc1ccc2[nH]nc(C)c2c1N(C)S(=O)(=O)c1cnn(-c2ccnc(C(F)(F)F)c2)c1]

SEM-deprotection was carried out following the same procedure as reported above. The crude product (70 mg) was purified by Prep-HPLC to afford the title compound (13 mg, 25%) as a white solid. (ES, m/z): [M+1]=467; ¹H NMR (400 MHz, DMSO-d₆) δ 2.62 (s, 3H), 3.27 (s, 3H), 3.36 (s, 3H), 7.15 (d, J=9.1 Hz, 1H), 7.45 (d, J=9.0 Hz, 1H), 8.22 (s, 1H), 8.32 (dd, J=5.5, 2.1 Hz, 1H), 8.54 (d, J=2.2 Hz, 1H), 8.90 (d, J=5.5 Hz, 1H), 9.51 (s, 1H), 12.61 (s, 1H).

### Example 128—Synthesis of N-(5-Methoxy-3-Methyl-1-((2-(Trimethylsilyl)Ethoxy)Methyl)-1H-Indazol-4-yl)-1-(4-(Trifluoromethyl)Pyridin-2-yl)-1H-Pyrazole-4-Sulfonamide

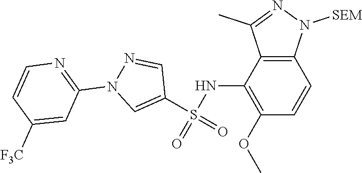 [SMILES: COc1ccc2c(c(C)nn2COCC[Si](C)(C)C)c1NS(=O)(=O)c1cnn(-c2cc(C(F)(F)F)ccn2)c1]

The title compound was obtained using general procedure A as a white solid (520 mg, 78%). (ES, m/z): [M+H]⁺ 583.

### Example 129—Synthesis of N-(5-Methoxy-3-Methyl-1H-Indazol-4-yl)-1-(4-(Trifluoromethyl)Pyridin-2-yl)-1H-Pyrazole-4-Sulfonamide (I-81)

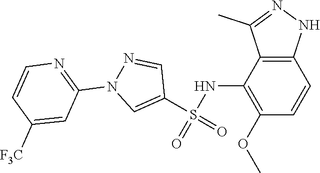 [SMILES: COc1ccc2[nH]nc(C)c2c1NS(=O)(=O)c1cnn(-c2cc(C(F)(F)F)ccn2)c1]

SEM-deprotection was carried out following the same procedure as reported above. The crude product was purified by prep-HPLC to afford the title compound (13 mg, 24%) as a white solid. (ES, m/z): [M+H]⁺ 453; ¹H NMR (400 MHz, DMSO-d₆) δ 2.64 (s, 3H), 3.34 (s, 3H), 7.08 (d, J=9.0 Hz, 1H), 8.06 (s, 1H), 8.19 (s, 1H), 8.77 (s, 1H), 8.82 (d, J=5.1 Hz, 1H), 9.76 (s, 1H), 12.56 (s, 1H).

### Example 130—Synthesis of N-(5-Methoxy-3-Methyl-1-((2-(Trimethylsilyl)Ethoxy)Methyl)-1H-Indazol-4-yl)-N-Methyl-1-(4-(Trifluoromethyl)Pyridin-2-yl)-1H-Pyrazole-4-Sulfonamide

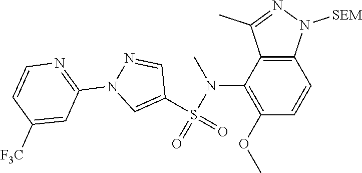 [SMILES: COc1ccc2c(c(C)nn2COCC[Si](C)(C)C)c1N(C)S(=O)(=O)c1cnn(-c2cc(C(F)(F)F)ccn2)c1]

The title compound was obtained using general procedure E as a colorless oil (178 mg, 87%). (ES, m/z): [M+H]+ 597.

### Example 131—Synthesis of N-(5-Methoxy-3-Methyl-1H-Indazol-4-yl)-N-Methyl-1-(4-(Trifluoromethyl)Pyridin-2-yl)-1H-Pyrazole-4-Sulfonamide (I-82)

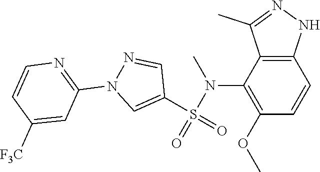 [SMILES: COc1ccc2[nH]nc(C)c2c1N(C)S(=O)(=O)c1cnn(-c2cc(C(F)(F)F)ccn2)c1]

SEM-deprotection was carried out following the same procedure as reported above. The crude product was purified by prep-HPLC to afford the title compound (49 mg, 41%) as a white solid. (ES, m/z): [M+H]⁺ 467; ¹H NMR (400 MHz, DMSO-d₆) δ 2.62 (s, 3H), 3.26 (s, 3H), 3.41 (s, 3H), 7.16 (d, J=9.1 Hz, 1H), 7.46 (d, J=9.0 Hz, 1H), 7.89 (dd, J=1.6, 5.2 Hz, 1H), 8.22 (d, J=8.8 Hz, 2H), 8.85 (d, J=5.1 Hz, 1H), 8.98 (s, 1H), 12.62 (s, 1H).

### Example 132—Synthesis of N-(6-Methoxy-1-Methyl-1H-Indazol-7-yl)-3-Methyl-1-(4-(Trifluoromethyl)Pyridin-2-yl)-1H-Pyrazole-4-Sulfonamide (I-83)

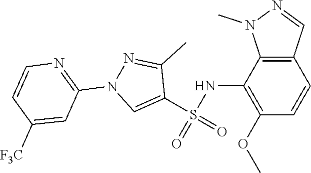 [SMILES: COc1ccc2cnn(C)c2c1NS(=O)(=O)c1cn(-c2cc(C(F)(F)F)ccn2)nc1C]

The title compound was obtained using general procedure A as an off-white solid (24 mg, 11%). (ES, m/z): [M+H]⁺ 467; ¹H NMR (400 MHz, DMSO-d₆) δ 2.36 (s, 3H), 3.32 (s, 3H), 4.26 (s, 3H), 6.84 (d, J=8.9 Hz, 1H), 7.67 (d, J=8.8 Hz, 1H), 7.81 (dd, J=1.5, 5.2 Hz, 1H), 7.99 (s, 1H), 8.10 (s, 1H), 8.54 (s, 1H), 8.75 (d, J=5.1 Hz, 1H), 9.74 (s, 1H).

### Example 133—Synthesis of N-(6-Methoxy-1-Methyl-1H-Indazol-7-yl)-5-Methyl-1-(4-(Trifluoromethyl)Pyridin-2-yl)-1H-Pyrazole-4-Sulfonamide (I-84)

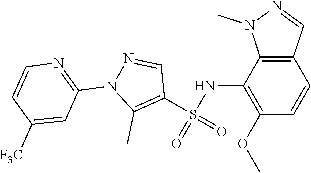 [SMILES: COc1ccc2cnn(C)c2c1NS(=O)(=O)c1cnn(-c2cc(C(F)(F)F)ccn2)c1C]

The title compound was obtained using general procedure A as a white solid (4 mg, 2%). (ES, m/z): [M+H]⁺ 401; ¹H NMR (400 MHz, DMSO-d6) δ 2.55 (s, 3H), 3.34 (s, 3H), 4.26 (s, 3H), 6.86 (d, J=8.9 Hz, 1H), 7.68 (d, J=8.8 Hz, 1H), 7.81 (s, 1H), 7.86-7.94 (m, 1H), 7.98 (s, 1H), 8.13 (dd, J=0.9, 1.6 Hz, 1H), 8.87 (d, J=5.1 Hz, 1H), 9.74 (s, 1H).

### Example 134—Synthesis of N-(6-chloro-1-Methyl-1H-Indazol-7-yl)-1-(4-(Trifluoromethyl)Pyridin-2-yl)-1H-Pyrazole-4-Sulfonamide (I-85)

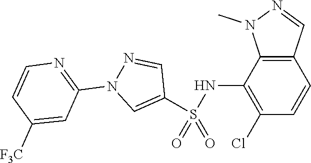 [SMILES: Cn1ncc2ccc(Cl)c(NS(=O)(=O)c3cnn(-c4cc(C(F)(F)F)ccn4)c3)c21]

The title compound was obtained using general procedure A as a white solid (12 mg, 7%). (ES, m/z): [M+H]⁺ 457; ¹H NMR (400 MHz, DMSO-d₆) δ 4.30 (s, 3H), 7.13 (d, J=8.5 Hz, 1H), 7.75 (d, J=8.5 Hz, 1H), 7.88 (dd, J=5.1, 1.6 Hz, 1H), 8.13 (d, J=7.8 Hz, 2H), 8.20 (s, 1H), 8.83 (m, 2H), 10.51 (s, 1H).

### Example 135—Synthesis of 1-(4-Chloropyridin-2-yl)-N-(1-Methyl-1H-Indazol-7-yl)-1H-Pyrazole-4-Sulfonamide (I-86)

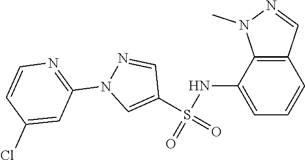 [SMILES: Cn1ncc2cccc(NS(=O)(=O)c3cnn(-c4cc(Cl)ccn4)c3)c21]

The title compound was obtained using general procedure C as a white solid (62 mg, 44%). (ES, m/z): [M+H]⁺ 390; ¹H NMR (400 MHz, DMSO-d₆) δ 4.28 (s, 3H), 6.72 (dd, J=7.2, 0.8 Hz, 1H), 6.97 (t, J=7.6 Hz, 1H), 7.64 (dd, J=5.6, 2.0 Hz, 1H), 7.69 (d, J=7.6 Hz, 1H), 8.03 (d, J=1.6 Hz, 1H), 8.06 (s, 1H), 8.09 (s, 1H), 8.52 (d, J=5.2 Hz, 1H), 8.80 (s, 1H), 10.06 (s, 1H).

### Example 136—Synthesis of N-(6-Methoxy-1-Methyl-1H-Pyrazolo[4,3-C]Pyridin-7-yl)-1-(2-(Trifluoromethyl)Pyridin-4-yl)-1H-Pyrazole-4-Sulfonamide (I-87)

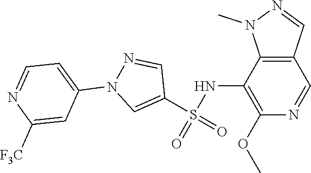 [SMILES: COc1ncc2cnn(C)c2c1NS(=O)(=O)c1cnn(-c2ccnc(C(F)(F)F)c2)c1]

The title compound was obtained using general procedure A as a white solid (3 mg, 2%). (ES, m/z): [M+H]⁺ 454; ¹H NMR (400 MHz, DMSO-d₆) δ 3.33 (s, 3H), 4.22 (s, 3H), 8.13 (s, 1H), 8.20 (s, 1H), 8.26 (dd, J=5.6 Hz, 2.0 Hz, 1H), 8.45 (d, J=2.0 Hz, 1H), 8.62 (s, 1H), 8.87 (d, J=5.2 Hz, 1H), 9.29 (s, 1H).

### Example 137—Synthesis of 1-(2-Cyclopropylpyridin-4-yl)-N-(3-Methyl-1H-Indazol-4-yl)-1H-Pyrazole-4-Sulfonamide (I-88)

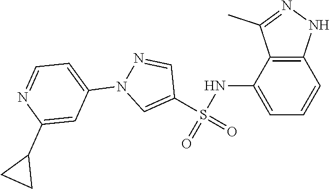 [SMILES: Cc1n[nH]c2cccc(NS(=O)(=O)c3cnn(-c4ccnc(C5CC5)c4)c3)c12]

The title compound was obtained using general procedure A as an off-white solid (7 mg, 32%). (ES, m/z): [M+H]⁺ 395; ¹H NMR (400 MHz, DMSO-d₆) δ 0.98 (dd, J=4.7, 7.1 Hz, 4H), 2.12-2.19 (m, 1H), 2.59 (s, 3H), 6.55 (d, J=7.3 Hz, 1H), 7.16 (t, J=7.9 Hz, 1H), 7.30 (s, 1H), 7.66 (dd, J=2.2, 5.6 Hz, 1H), 7.84 (d, J=2.2 Hz, 1H), 8.01 (s, 1H), 8.47 (d, J=5.6 Hz, 1H), 9.13 (s, 1H), 9.93 (s, 1H), 12.69 (s, 1H).

### Example 138—Synthesis of 1-(4-Cyclopropylpyridin-2-yl)-N-(3-Methyl-1H-Indazol-4-yl)-1H-Pyrazole-4-Sulfonamide (I-89)

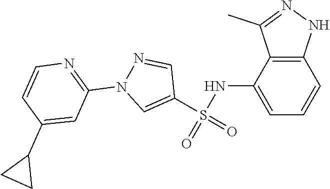 [SMILES: Cc1n[nH]c2cccc(NS(=O)(=O)c3cnn(-c4cc(C5CC5)ccn4)c3)c12]

The title compound was obtained using general procedure A as an off-white solid (4 mg, 27%). (ES, m/z): [M+H]⁺ 395; ¹H NMR (400 MHz, DMSO-d₆) δ 0.89-0.92 (m, 2H), 1.09-1.19 (m, 2H), 2.12 (dt, J=3.7, 8.2 Hz, 1H), 2.59 (s, 3H), 6.55 (d, J=7.3 Hz, 1H), 7.15 (s, 2H), 7.28 (s, 1H), 7.66 (s, 1H), 7.97 (s, 1H), 8.30 (d, J=5.2 Hz, 1H), 8.73 (s, 1H), 9.89 (s, 1H), 12.69 (s, 1H).

### Example 139—Synthesis of N-(6-Methoxy-1-Methyl-1H-Indazol-7-yl)-1-(5-Methoxy-2-(Trifluoromethyl)Pyridin-4-yl)-1H-Pyrazole-4-Sulfonamide (I-90)

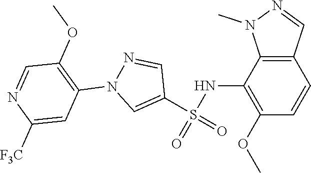 [SMILES: COc1cnc(C(F)(F)F)cc1-n1cc(S(=O)(=O)Nc2c(OC)ccc3cnn(C)c23)cn1]

The title compound was obtained using general procedure D as a white solid (22 mg, 12%). (ES, m/z): [M+H]^{m} 483; ¹H NMR (400 MHz, DMSO-d₆) δ 3.35 (s, 3H), 4.11 (s, 3H), 4.24 (s, 3H), 6.90 (d, J=8.9 Hz, 1H), 7.70 (d, J=8.8 Hz, 1H), 7.98 (s, 1H), 8.07 (s, 1H), 8.20 (s, 1H), 8.59 (s, 1H), 8.84 (s, 1H), 9.74 (s, 1H).

### Example 140—Synthesis of 6-(1-Ethoxyvinyl)-1-Methyl-7-Nitro-1H-Indazole

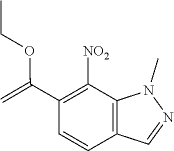 [SMILES: C=C(OCC)c1ccc2cnn(C)c2c1[N+](=O)[O-]]

To a stirred solution of 6-chloro-1-methyl-7-nitro-1H-indazole (500 mg, 2.4 mmol, 1.0 eq.) and tributyl(1-ethoxyvinyl)tin (0.96 mL, 2.8 mmol, 1.2 eq.) under nitrogen in 1,4-dioxane (12 mL) was added bis(triphenylphosphine)palladium(II) dichloride (166 mg, 0.2 mmol, 0.1 eq.) and stirred at 100° C. overnight. The reaction mixture was diluted with EtOAc (6 mL), washed with water (3×6 mL), dried over magnesium sulphate and concentrated under reduced pressure. The crude residue was purified by silica gel chromatography eluting with 0-50% EtOAc in cyclohexane to yield the title compound. ¹H NMR (400 MHz, DMSO-d₆) δ 1.24 (3H, dd, J=7.0, 7.0 Hz), 3.86 (2H, q, J=7.0 Hz), 3.92 (3H, s), 4.58 (11H, d, J=3.0 Hz), 4.66 (11H, d, J=3.0 Hz), 7.37 (1H, d, J=8.3 Hz), 8.06 (1H, d, J=8.4 Hz), 8.34 (1H, s).

### Example 141—Synthesis of 1-(7-Amino-1-Methyl-1H-Indazol-6-yl)Ethan-1-One

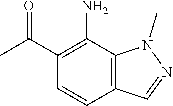

A stirred mixture of 1-(1-methyl-7-nitro-1H-indazol-6-yl)ethan-1-one (100 mg, 0.5 mmol, 1.0 eq.), ammonium chloride (122 mg, 2.3 mmol, 5.0 eq.) and iron powder (255 mg, 4.6 mmol, 10 eq.) in EtOH (6 mL) and water (2 mL) was stirred at 60° C. overnight. The reaction mixture was diluted with EtOAc (10 mL) and filtered through celite. The filter cake was washed with EtOAc (3×5 mL) and the solvent removed under reduced pressure to yield the title compound as an off-white solid (86 mg, 99%). (ES, m/z): [M+H]⁺ 190.

### Example 142—Synthesis of 6-(Ethyl-1,1-D2)-1-Methyl-1H-Indazol-7-Amine

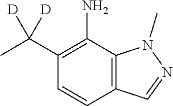

To a mixture of 1-(1-methyl-7-nitroindazol-6-yl)ethanone (1.0 g, 4.6 mmol, 1.0 eq.), AlCl₃ (3.65 g, 27 mmol, 6.0 eq.) and THE (50 mL) was added LiAlD4 (1.15 g, 27 mmol, 6.0 eq.) at 0° C. under nitrogen. The resulting mixture was stirred for 16 h at 60° C. under nitrogen. The reaction was quenched by the addition of sat. NH₄Cl (20 mL) at 0° C., and the resulting mixture was extracted with EtOAc (3×30 mL). The combined organic layers were washed with brine (10 mL), dried over anhydrous Na₂SO₄, and concentrated under reduced pressure. The residue was purified by silica gel column chromatography, eluted with PE/EA (1:1) to afford the title compound (250 mg, 31%) as a yellow oil. (ES, m/z): [M+H]⁺ 178.

### Example 143—Synthesis of N-(4-Methoxybenzyl)-1-Methyl-1H-Pyrazolo[4,3-C]Pyridin-7-Amine

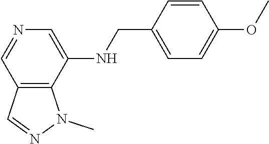 [SMILES: COc1ccc(CNc2cncc3cnn(C)c23)cc1]

To a stirred mixture of 7-bromo-1-methylpyrazolo[4,3-c]pyridine (750 mg, 3.6 mmol, 1.0 eq.) and Cs₂CO₃ (2.3 g, 7.0 mmol, 2.0 eq.), Pd(OAc)₂ (119 mg, 0.5 mmol, 0.15 eq.), XantPhos (307 mg, 0.5 mmol, 0.15 eq.) and 1,4-dioxane (30 mL) was added (4-methoxyphenyl) methanamine (631 mg, 4.6 mmol, 1.3 eq.) dropwise at room temperature under nitrogen. The resulting mixture was stirred for 2 h at 100° C. under nitrogen, and then was quenched by the addition of water (10 mL) at 0° C. The resulting mixture was extracted with EtOAc (3×30 mL). The combined organic layers were washed with brine (10 mL), dried over Na₂SO₄, and concentrated under reduced pressure. The crude product (1.2 g) was purified by reversed-phase flash chromatography with the following conditions: column: C18 silica gel; mobile phase: MeCN in water (10 mmol/L NH₄CO₃), 10% to 30% gradient in 10 min; detector: UV 220 nm. This resulted the title compound (870 mg, 92%) as a brown liquid. (ES, m/z): [M+H]⁺ 269; ¹H NMR (400 MHz, DMSO-d₆) δ 3.74 (s, 3H), 4.45-4.49 (m, 2H), 4.50 (s, 3H), 6.83-6.99 (m, 2H), 7.36-7.51 (m, 3H), 8.59 (s, 1H), 8.90 (s, 1H).

### Example 144—Synthesis of 1-Methyl-1H-Pyrazolo[4,3-C]Pyridin-7-Amine

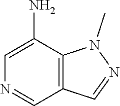

To a stirred solution of N-[(4-methoxyphenyl)methyl]-1-methylpyrazolo[4,3-c]pyridin-7-amine (1.0 g, 3.7 mmol, 1.0 eq.) in DCM (7.5 mL) were added TFA (2.5 mL, 34 mmol, 9.0 eq.) dropwise at room temperature under nitrogen. The resulting mixture was stirred for 2 h at room temperature under nitrogen, and then concentrated under reduced pressure. The residue was purified by reverse flash chromatography with the following conditions: column: C18 silica gel; mobile phase: MeCN in water (10 mmol/L NH₄CO₃), 10% to 50% gradient in 40 min; detector: UV 220 nm. This resulted in the title compound (207 mg, 37%) as a yellow solid. (ES, m/z): [M+H]⁺ 149; ¹H NMR (400 MHz, DMSO-d₆) δ 4.25 (s, 3H), 5.32 (s, 2H), 7.71 (s, 1H), 8.05 (s, 1H), 8.37 (s, 1H).

### Example 145—Synthesis of 6-Chloro-1-Methyl-7-Nitropyrazolo[4,3-C] Pyridin-3-ol

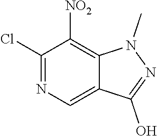

A solution of ethyl 4,6-dichloro-5-nitropyridine-3-carboxylate (500 mg, 1.9 mmol, 1 eq.), Et₃N (573 mg, 5.7 mmol, 3 eq.) and methylhydrazine sulfuric acid (299 mg, 2.1 mmol, 1.1 eq.) in EtOH (15 mL) was stirred overnight at room temperature, and then concentrated under reduced pressure. The resulting residue was diluted with H₂O (20 mL) and extracted with EtOAc (2×20 mL). The combined organic layers were washed with brine (50 mL), dried over Na₂SO₄, and concentrated under reduced pressure. The residue was purified by reversed-phase flash chromatography with the following conditions: column: C18 silica gel; mobile phase: MeCN in water, 0% to 100% gradient in 20 min; detector: UV 254 nm, to afford the title compound (180 mg, 41%) as red solid. (ES, m/z): [M+H]⁺=229.

### Example 146—Synthesis of 6-Methoxy-1-Methyl-7-Nitropyrazolo[4,3-C] Pyridin-3-ol

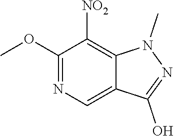 [SMILES: COc1ncc2c(O)nn(C)c2c1[N+](=O)[O-]]

A solution of 6-chloro-1-methyl-7-nitropyrazolo[4,3-c] pyridin-3-ol (180 mg, 0.8 mmol, 1 eq.) and MeONa (425 mg, 7.9 mmol, 10 eq.) in MeOH (8 mL) was stirred overnight at 60° C. The mixture was allowed to warm to room temperature, and was then concentrated under reduced pressure. The resulting residue was diluted with H₂O (20 mL) and extracted with EtOAc (2×20 mL). The combined organic layers were washed with brine (50 mL), dried over Na₂SO₄, and concentrated under reduced pressure. The residue was purified by reversed-phase flash chromatography with the following conditions: column: C18 silica gel; mobile phase: MeCN in water, 0% to 100% gradient in 20 min; detector: UV 254 nm, to afford the title compound (84 mg, 43%) as a yellow solid. (ES, m/z): [M+H]⁺=225.

### Example 147—Synthesis of 6-Methoxy-1-Methyl-7-Nitropyrazolo[4,3-C] Pyridin-3-yl Trifluoromethanesulfonate

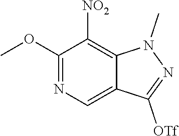 [SMILES: COc1ncc2c(OS(=O)(=O)C(F)(F)F)nn(C)c2c1[N+](=O)[O-]]

A solution of 6-methoxy-1-methyl-7-nitropyrazolo[4,3-c] pyridin-3-ol (60 mg, 0.27 mmol, 1 eq.), pyridine (42 mg, 0.54 mmol, 2 eq.) and Tf₂O (94 mg, 0.34 mmol, 1.25 eq.) in DCM (8 mL) was stirred overnight from 0° C. to room temperature. The resulting mixture was diluted with H₂O (20 mL) and extracted with EtOAc (2×20 mL). The combined organic layers were washed with brine (50 mL), dried over Na₂SO₄, and concentrated under reduced pressure. The residue was purified by Prep-TLC (PE/EA, 7:1) to afford the title compound (66 mg, 68%) as an orange oil. (ES, m/z): [M+H]⁺=357.

### Example 148—Synthesis of 6-Methoxy-1-Methylpyrazolo[4,3-C]Pyridin-7-Amine

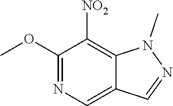

A solution of 6-methoxy-1-methyl-7-nitropyrazolo[4,3-c]pyridine-3-yl trifluoromethane sulfonate (80 mg, 0.23 mmol, 1 eq.) and Pd/C (72 mg, 0.68 mmol, 3 eq.) in MeOH (10 mL) was stirred overnight at room temperature under hydrogen. The resulting mixture was filtered, washed with MeOH (20 ml), and the filtrate was concentrated under reduced pressure to give the title compound (40 mg, 95%) as a dark green oil. (ES, m/z): [M+H]⁺=179.

### Example 149—Synthesis of N-(3-Fluoro-1-Methyl-1H-Indazol-7-yl)-1,1-Diphenylmethanimine

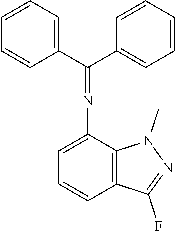 [SMILES: Cn1nc(F)c2cccc(N=C(c3ccccc3)c3ccccc3)c21]

A solution of 7-bromo-3-fluoro-1-methyl-1H-indazole (130 mg, 0.6 mmol, 1 eq.), Pd₂(dba)₃ (104 mg, 0.1 mmol, 0.2 eq.), XantPhos (82 mg, 0.14 mmol, 0.25 eq.), Cs₂CO₃ (555 mg, 1.7 mmol, 3.0 eq.) and diphenylmethanimine (123 mg, 0.7 mmol, 1.2 eq.) in dioxane (2 mL) was stirred for 2 h at 80° C. under nitrogen. The resulting mixture was then diluted with water (10 mL), and was extracted with EtOAc (3×10 mL). The combined organic layers were washed with brine (10 mL), dried over Na₂SO₄, and concentrated under reduced pressure. The residue was purified by silica gel column chromatography and eluted with PE/EtOAc (5:1) to afford the title compound (120 mg, quantitative) as a light yellow solid. (ES, m/z): [M+H]⁺ 330.

### Example 150—Synthesis of 3-Fluoro-1-Methyl-1H-Indazol-7-Amine

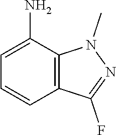

To a solution of N-(3-fluoro-1-methyl-1H-indazol-7-yl)-1,1-diphenylmethanimine (130 mg, 0.4 mmol, 1.0 eq.) in dioxane (4 mL) was added HCl (5 N, 4 mL) dropwise and stirred for 30 min at room temperature under nitrogen. The reaction mixture was basified to pH 8 with saturated NaHCO₃. The resulting mixture was then extracted with EtOAc (3×10 mL), and the combined organic layers were washed with brine (10 mL), dried over Na₂SO₄, and concentrated under reduced pressure. The residue was purified by silica gel column chromatography and eluted with PE/EtOAc (5:1) to afford the title compound (60 mg, quant.) as a light-yellow solid. (ES, m/z): [M+H]⁺ 166.

### Example 151—Synthesis of 6-Chloro-1-Methyl-4-Nitropyrazolo[4,3-C]Pyridine

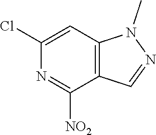

To a mixture of 6-chloro-1-methylpyrazolo[4,3-c]pyridine (500 mg, 3.0 mmol, 1.0 eq.) in H₂SO₄ (3 mL) was added HNO₃ (752 mg, 12 mmol, 4.0 eq.) at 0° C. The resulting solution was stirred at room temperature for 16 h. After the reaction was complete, the mixture was added to ice water (50 mL), and extracted with EtOAc (3×30 mL). The combined organic layers were washed with brine (50 mL), dried over Na₂SO₄, and concentrated under reduced pressure to give the title compound (500 mg, 79%) as a yellow solid. (ES, m/z): [M+H]⁺=213.

### Example 152—Synthesis of 6-Ethenyl-1-Methyl-4-Nitropyrazolo[4,3-C]Pyridine

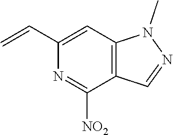

A solution of 6-chloro-1-methyl-4-nitropyrazolo[4,3-c]pyridine (491 mg, 2.3 mmol, 1.0 eq.), potassium ethenyltrifluoroboranuide (371 mg, 2.8 mmol, 1.2 eq.), Pd(dppf)Cl₂ (338 mg, 0.5 mmol, 0.2 eq.) and K₂CO₃ (638 mg, 4.6 mmol, 2.0 eq.) in 1,4-dioxane (15 mL) was stirred for 16 h at 100° C. under nitrogen. The mixture was added to water (50 mL) and extracted with EtOAc (3×30 mL). The combined organic layers were washed with brine (50 mL), dried over Na₂SO₄, and concentrated under reduced pressure. The residue was purified by reversed-phase flash chromatography with the following conditions: column: C18 silica gel; mobile phase: MeCN in water, 0% to 100% gradient in 20 min; detector: UV 254 nm, to afford the title compound (111 mg, 24%) as a yellow solid. (ES, m/z): [M+H]⁺=205; ¹H NMR (400 MHz, DMSO-d₆) δ 4.20 (s, 3H), 5.58 (d, J=1.6 Hz, 1H), 6.40 (d, J=1.6 Hz, 1H), 6.94 (d, J=6.8 Hz, 1H), 7.94 (s, 1H), 9.31 (s, 1H).

### Example 153—Synthesis of 6-Ethyl-1-Methylpyrazolo[4,3-C]Pyridin-4-Amine

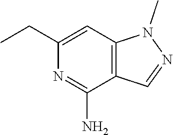

A solution of 6-ethenyl-1-methyl-4-nitropyrazolo[4,3-c]pyridine (111 mg, 0.5 mmol, 1.0 eq.) and Pd/C (174 mg, 1.6 mmol, 3.0 eq.) in MeOH (12 mL) was stirred for 16 h at room temperature under hydrogen. The resulting mixture was filtered and the filter cake was washed with MeOH (20 mL). The filtrate was concentrated under reduced pressure to afford the title compound (90 mg, 94%) as yellow oil. (ES, m/z): [M+H]⁺=177.

### Example 154—Synthesis of 7-Bromo-3-Methoxy-2-Methyl-2H-Indazole and 7-Bromo-3-Methoxy-1-Methyl-1H-Indazole

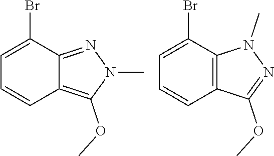

A mixture of 7-bromo-1H-indazol-3-ol (1 g, 4.7 mmol, 1.0 eq.) and Cs₂CO₃ (4.6 g, 14 mmol, 3.0 eq.) in THE (40 mL) was stirred for 30 min at room temperature. CH₃I (2.0 g, 14 mmol, 3.0 eq.) was then added in portions over 1 min at room temperature. The resulting mixture was stirred for an additional 2 h at room temperature. The resulting mixture was then extracted with EtOAc (2×200 mL). The combined organic layers were washed with brine (100 mL), dried over anhydrous Na₂SO₄, and concentrated under reduced pressure. The residue was purified by silica gel column chromatography, eluting with PE/EA (10:1) to afford the title compounds. 7-Bromo-3-methoxy-2-methyl-2H-indazole (530 mg, 47%.) as a yellow solid; (ES, m/z): [M+H]+ 241; 7-Bromo-3-Methoxy-1-Methyl-1H-Indazole (140 mg, 12%) as a yellow solid; (ES, m/z): [M+H]⁺ 241.

### Example 155—Synthesis of 1-Methyl-7-Nitro-6-Vinyl-1H-Pyrazolo[4,3-C]Pyridin-3-ol

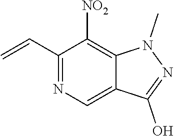 [SMILES: C=Cc1ncc2c(O)nn(C)c2c1[N+](=O)[O-]]

A solution of 6-chloro-1-methyl-7-nitropyrazolo[4,3-c]pyridin-3-ol (623 mg, 2.7 mmol, 1 eq.), potassium ethenyltrifluoroboranuide (493 mg, 2.8 mmol, 1.2 eq.), Pd(dppf)Cl₂ (399 mg, 0.5 mmol, 0.2 eq.) and K₂CO₃ (753 mg, 5.5 mmol, 2 eq.) in 1,4-dioxane (21 mL) was stirred overnight at 100° C. under nitrogen. The resulting mixture was extracted with EtOAc (3×30 mL). The combined organic layers were washed with brine (50 mL), dried over anhydrous Na₂SO₄, and concentrated under reduced pressure. The mixture was then purified by reversed-phase flash chromatography with the following conditions: column: C18 silica gel; mobile phase: MeCN in water, 0% to 100% gradient in 25 min; detector: UV 254 nm, to afford the title compound (300 mg, 50%) yellow solid as product. (ES, m/z): [M+H]⁺ 221.

### Example 156—Synthesis of 1-Methyl-7-Nitro-6-Vinyl-1H-Pyrazolo[4,3-C]Pyridin-3-yl Trifluoromethanesulfonate

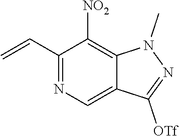 [SMILES: C=Cc1ncc2c(OS(=O)(=O)C(F)(F)F)nn(C)c2c1[N+](=O)[O-]]

A solution of 1-methyl-7-nitro-6-vinyl-1H-pyrazolo[4,3-c]pyridin-3-ol (0.6 g, 2.7 mmol, 1 eq.), trifluoromethanesulfonic anhydride (1.9 g, 6.75 mmol, 2.5 eq.) and pyridine (0.4 g, 5.5 mmol, 2.0 eq.) in DCM (27 mL) was stirred overnight at room temperature. The resulting mixture was diluted with water (20 ml) and extracted with DCM (3×30 mL). The combined organic layers were washed with brine (50 mL), dried over Na₂SO₄, and concentrated under reduced pressure. The reaction mixture was purified by Prep-TLC (PE/EA 8:1) to afford the title compound (145 mg, 15%) as a yellow solid. (ES, m/z): [M+H]⁺ 353.

### Example 157—Synthesis of 6-Ethyl-1-Methyl-1H-Pyrazolo[4,3-C]Pyridin-7-Amine

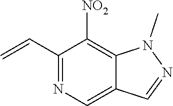

A solution of 6-ethenyl-1-methyl-7-nitropyrazolo[4,3-c]pyridin-3-yl trifluoromethanesulfonate (180 mg, 1.0 mmol, 1 eq.) and Pd/C (32 mg, 0.3 mmol, 0.3 eq.) in MeOH (10 mL) was stirred overnight at room temperature under hydrogen. The reaction mixture was then filtered, and the filtrate was concentrated under vacuum to afford the title compound (70 mg, 78%) as a white solid. (ES, m/z): [M+H]⁺ 177. ¹H NMR (400 MHz, MeOH-d₄) δ 8.64 (s, 1H), 8.40 (s, 1H), 4.46 (d, J=4.0 Hz, 3H), 3.31-3.30 (m, 2H), 1.33 (t, J=7.6 Hz, 3H).

### Example 158—Synthesis of 2,5-Dimethyl-1H-indazol-6-Amine

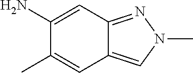

A solution of 2,5-dimethyl-6-nitro-1H-indazole (300 mg, 1.6 mmol, 1 eq.) and Pd/C (83 mg, 0.8 mmol, 0.5 eq.) in MeOH (10 mL) was stirred for 2 h at room temperature under hydrogen. The resulting mixture was filtered, the solids were washed with EtOH (3×10 mL), and the filtrate was concentrated under reduced pressure. This resulted in the title compound (165 mg, 57%) as an off-white solid. (ES, m/z): [M+H]⁺ 162.

### Example 159—Synthesis of 3-(2-Fluoropyridin-4-yl)Oxetan-3-ol

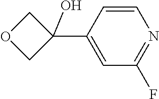 [SMILES: OC1(c2ccnc(F)c2)COC1]

A solution of 4-bromo-2-fluoropyridine (3.0 g, 17.0 mmol, 1 eq.) in THE (30 mL) was treated with i-PrMgCl (1 M in THF, 19 mL, 19 mmol, 1.1 eq.) for 2 h at −78° C. under nitrogen, followed by the addition of 3-oxetanone (1.8 g, 26 mmol, 1.5 eq.) dropwise at −78° C. The resulting mixture was stirred for 4 h at room temperature under nitrogen, and then was quenched with sat. NH₄Cl (10 mL), and extracted with EtOAc (3×10 mL). The combined organic layers were washed with brine (15 mL), dried over Na₂SO₄, and concentrated under reduced pressure. The resulting residue was purified by reversed-phase flash chromatography with the following conditions: column: C18 silica gel; mobile phase: MeCN in water (0.1% TFA), 0% to 0% gradient in 10 min; detector: UV 254 nm. This resulted in the title compound (300 mg, 10%) as a white solid. (ES, m/z): [M+H]⁺ 170.

### Example 160—Synthesis of 2-Fluoro-4-(3-Fluorooxetan-3-yl)Pyridine

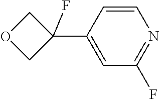 [SMILES: Fc1cc(C2(F)COC2)ccn1]

To a stirred solution of 3-(2-fluoropyridin-4-yl)oxetan-3-ol (1.2 g, 7 mmol, 1 eq.) in DCM (45 mL) was added DAST (2.2 g, 14 mmol, 2 eq.) dropwise at −78° C. under nitrogen. The resulting mixture was stirred under nitrogen for 30 min at −78° C. and for another 1 h at room temperature. The resulting mixture was concentrated under reduced pressure, and the resulting residue was purified by reversed-phase flash chromatography with the following conditions: column: C18 silica gel; mobile phase: MeCN in water (0.1% TFA), 10% to 50% gradient in 10 min; detector: UV 254 nm. This resulted in the title compound (205 mg, 18%) as a light brown oil. (ES, m/z): [M+H]⁺ 172.

### Example 161—Synthesis of N-(1-Methyl-1H-Indazol-7-yl)-1-(5-(Trifluoromethyl)Pyridin-2-yl)-1H-Pyrazole-4-Sulfonamide (I-147)

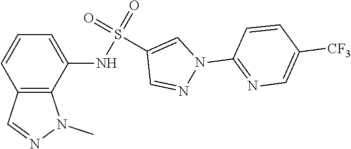 [SMILES: Cn1ncc2cccc(NS(=O)(=O)c3cnn(-c4ccc(C(F)(F)F)cn4)c3)c21]

The title compound was obtained using general procedure A as an off white solid (46 mg, 54%). (ES, m/z): [M+H]⁺ 423.2; ¹H NMR (400 MHz, DMSO-d₆) δ 4.28 (s, 3H), 6.73 (d, J=7.3 Hz, 1H), 7.00-6.95 (m, 1H), 7.70 (d, J=7.8 Hz, 1H), 8.10 (d, J=9.9 Hz, 2H), 8.18 (d, J=8.7 Hz, 1H), 8.49 (dd, J=2.1, 8.7 Hz, 1H), 8.86 (s, 1H), 8.96 (m, 1H), 10.10 (s, 1H).

### Example 162—Synthesis of N-(5-Methyl-1H-Indazol-4-yl)-1-(5-(Trifluoromethyl)Pyridin-2-yl)-1H-Pyrazole-4-Sulfonamide (I-148)

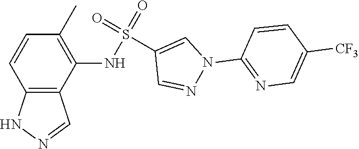 [SMILES: Cc1ccc2[nH]ncc2c1NS(=O)(=O)c1cnn(-c2ccc(C(F)(F)F)cn2)c1]

The title compound was obtained using general procedure A as an off white solid (14 mg, 26%). (ES, m/z): [M+H]⁺ 423.3; ¹H NMR (400 MHz, DMSO-d₆) δ 2.23-2.22 (m, 3H), 7.23-7.19 (m, 1H), 7.38-7.33 (m, 1H), 7.59 (s, 1H), 8.01-8.00 (m, 1H), 8.15-8.12 (m, 1H), 8.50-8.45 (m, 1H), 8.68-8.67 (m, 1H), 8.94-8.93 (m, 1H), 10.03-9.98 (m, 1H), 12.92 (s, 1H).

### Example 163—Synthesis of N-(1H-indazol-7-yl)-1-(5-(Trifluoromethyl)Pyridin-2-yl)-1H-Pyrazole-4-Sulfonamide (I-149)

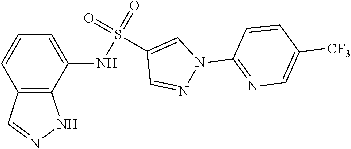 [SMILES: O=S(=O)(Nc1cccc2cn[nH]c12)c1cnn(-c2ccc(C(F)(F)F)cn2)c1]

The title compound was obtained using general procedure A as a brown solid (77 mg, 49%). (ES, m/z): [M+H]⁺ 409.1; H NMR (400 MHz, DMSO-d₆) δ 7.00 (t, J=7.7 Hz, 1H), 7.10 (d, J=7.4 Hz, 1H), 7.55-7.48 (m, 1H), 8.04 (s, 1H), 8.11 (t, J=10.3 Hz, 2H), 8.45 (dd, J=2.1, 8.7 Hz, 1H), 8.91 (s, 1H), 8.94 (t, J=1.1 Hz, 1H), 10.19 (s, 1H), 12.83 (s, 1H).

### Example 164—Synthesis of N-(1H-indazol-4-yl)-1-(5-(Trifluoromethyl)Pyridin-2-yl)-1H-Pyrazole-4-Sulfonamide (I-150)

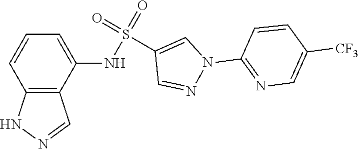 [SMILES: O=S(=O)(Nc1cccc2[nH]ncc12)c1cnn(-c2ccc(C(F)(F)F)cn2)c1]

The title compound was obtained using general procedure A as a pink solid (31 mg, 55%). (ES, m/z): [M+H]⁺ 409.2; ¹H NMR (400 MHz, DMSO-d₆) δ 7.05-7.02 (m, 1H), 7.27 (d, J=4.4 Hz, 2H), 8.08 (d, J=8.8 Hz, 1H), 8.17 (s, 1H), 8.24 (d, J=1.1 Hz, 1H), 8.45 (dd, J=2.1, 8.7 Hz, 1H), 8.95-8.92 (m, 2H), 10.67-10.64 (m, 1H), 13.11-13.07 (m, 1H).

### Example 165—Synthesis of N-(6-Acetyl-1-Methyl-1H-Indazol-7-yl)-1-(4-(Trifluoromethyl)Pyridin-2-yl)-1H-Pyrazole-4-Sulfonamide (I-151)

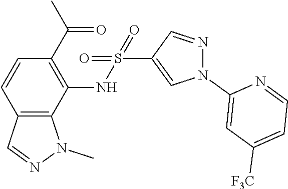 [SMILES: CC(=O)c1ccc2cnn(C)c2c1NS(=O)(=O)c1cnn(-c2cc(C(F)(F)F)ccn2)c1]

The title compound was obtained using general procedure A as an off-white solid (8 mg, 1%). (ES, m/z): [M+H]⁺ 465; ¹H NMR (400 MHz, DMSO-d₆) δ 2.27 (3H, s), 4.22 (3H, s), 7.35 (1H, d, J=8.6 Hz), 7.78 (1H, s), 7.87 (1H, d, J=5.1 Hz), 7.91 (1H, s), 8.17-8.13 (2H, m), 8.63 (1H, s), 8.80 (1H, d, J=5.1 Hz), 10.28 (1H, d, J=1.0 Hz).

### Example 166—Synthesis of N-(6-(1-Hydroxyethyl)-1-Methyl-1H-Indazol-7-yl)-1-(4-(Trifluoromethyl)Pyridin-2-yl)-1H-Pyrazole-4-Sulfonamide (I-152)

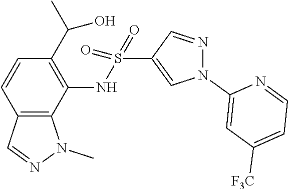 [SMILES: CC(O)c1ccc2cnn(C)c2c1NS(=O)(=O)c1cnn(-c2cc(C(F)(F)F)ccn2)c1]

To a stirred solution of N-(6-acetyl-1-Methyl-1H-Indazol-7-yl)-1-(4-(trifluoromethyl) pyridin-2-yl)-1H-pyrazole-4-sulfonamide (20 mg, 0.04 mmol, 1.0 eq.) in THE (1 mL) at 0° C. was added sodium borohydride (3.3 mg, 0.09 mmol, 2.0 eq.) and the mixture stirred at 0° C. for 20 min before warming to room temperature and stirring for a further 4 h. The reaction mixture was diluted with water (6 mL) and extracted with EtOAc (3×4 mL). The organic extract was washed with brine (6 mL), dried over magnesium sulphate, and the solvent removed under reduced pressure. The crude residue was purified by HPLC to yield the title compound as an off-white solid (2 mg, 10%). (ES, m/z): [M+H]⁺ 467; ¹H NMR (400 MHz, DMSO-d₆) δ 1.06 (3H, d, J=6.1 Hz), 4.84 (1H, s), 4.21 (3H, s), 7.18 (1H, d, J=8.3 Hz), 7.54-7.52 (1H, m), 7.83 (1H, d, J=5.3 Hz), 7.93 (1H, s), 8.01 (1H, d, J=1.0 Hz), 8.16 (1H, s), 8.65 (1H, s), 8.79 (1H, d, J=5.3 Hz). NH not observed.

### Example 167—Synthesis of 1-[2-Chloro-6-(Trifluoromethyl)-4-Pyridyl]-N-(6-Ethyl-1-Methyl-Indazol-7-yl)Pyrazole-4-Sulfonamide

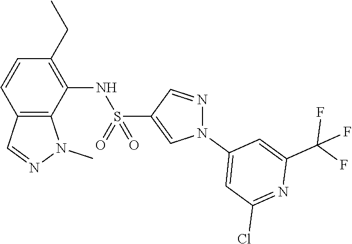 [SMILES: CCc1ccc2cnn(C)c2c1NS(=O)(=O)c1cnn(-c2cc(Cl)nc(C(F)(F)F)c2)c1]

To a solution of 6-ethyl-1-methyl-indazol-7-amine (35 mg, 0.2 mmol, 1.0 eq.) in pyridine (1 mL) was added a solution of 1-[2-chloro-6-(trifluoromethyl)-4-pyridyl]pyrazole-4-sulfonyl chloride (69 mg, 0.2 mmol, 1.0 eq.) in DCM (2 mL) and the resulting mixture was stirred at 20° C. for 3 d. The reaction mixture was concentrated under reduced pressure and the residue was partitioned between DCM (5 mL) and water (2 mL). The aqueous phase was separated and further extracted with DCM (5 mL). The combined organic extracts were dried over magnesium sulfate, filtered and concentrated under reduced pressure. The residue was purified by preparative HPLC to give the title compound (32 mg, 31%). (ES, m/z): [M+H]⁺ 484.9; ¹H NMR (400 MHz, CDCl₃) δ 1.02 (t, J=7.8 Hz, 3H), 2.25 (q, J=7.8 Hz, 2H), 4.38 (s, 3H), 6.57 (s, 1H), 6.97 (d, J=8.8 Hz, 1H), 7.68 (d, J=8.8 Hz, 1H), 7.80 (d, J=1.1 Hz, 1H), 7.89 (s, 1H), 7.9-7.91 (m, 1H), 7.97 (s, 1H), 8.18 (s, 1H).

### Example 168—Synthesis of N-(6-Ethyl-1-Methyl-Indazol-7-yl)-1-[2-Morpholino-6-(Trifluoromethyl)-4-Pyridyl]Pyrazole-4-Sulfonamide (I-153)

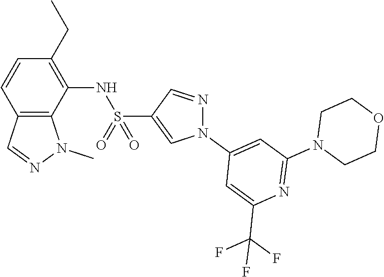 [SMILES: CCc1ccc2cnn(C)c2c1NS(=O)(=O)c1cnn(-c2cc(N3CCOCC3)nc(C(F)(F)F)c2)c1]

A solution of morpholine (17 μL, 0.2 mmol, 3.0 eq.) and 1-[2-chloro-6-(trifluoromethyl)-4-pyridyl]-N-(6-ethyl-1-methyl-indazol-7-yl)pyrazole-4-sulfonamide (31 mg, 0.06 mmol, 1.0 eq.) in 1-propanol (1 mL) was stirred at 130° C. for 5 d. Another portion of morpholine (17 μL, 0.2 mmol, 3.0 eq.) was added and stirring at 130° C. was continued for 3 h. The reaction mixture was concentrated in vacuo and the residue purified by preparative HPLC to give the title compound (10 mg, 29%) as an off white solid. (ES, m/z): [M+H]⁺ 536.2; ¹H NMR (400 MHz, DMSO-d₆) δ 0.87 (t, J=8.2 Hz, 3H), 2.30-2.36 (m, 2H), 3.52 (t, J=4.9 Hz, 4H), 3.73 (t, J=4.9 Hz, 4H), 4.24 (s, 3H), 6.92-6.89 (m, 1H), 7.30 (d, J=1.8 Hz, 1H), 7.44 (d, J=2.0 Hz, 2H), 7.86 (s, 1H), 7.89 (s, 1H), 8.38 (s, 1H), 8.48 (s, 1H).

### Example 169—Synthesis of N-(6-Ethyl-1-Methyl-Indazol-7-yl)-3,5-Dimethyl-1-[5-(Trifluoromethyl)-2-Pyridyl]Pyrazole-4-Sulfonamide (I-154)

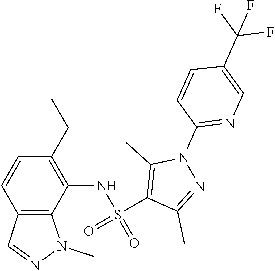

A solution of 6-ethyl-1-methyl-indazol-7-amine (30 mg, 0.2 mmol, 1.0 eq.), pyridine (0.04 mL, 0.5 mmol, 3.0 eq.), and 4-(dimethylamino)pyridine (2.1 mg, 0.02 mmol, 0.1 eq.) in DCM (0.6 mL) was cooled to 0° C. and to it was added 3,5-dimethyl-1-[5-(trifluoromethyl)-2-pyridyl]pyrazole-4-sulfonyl chloride (70 mg, 0.2 mmol, 1.2 eq.) and the mixture stirred at 50° C. for 72 h. The reaction mixture was allowed to cool down to room temperature before diluting with water (10 mL). The product was extracted using DCM (3×15 mL). The organics were combined, dried over sodium sulfate, filtered, and concentrated. The crude product was purified by preparative HPLC to yield the title compound (45 mg, 54%) as an off-white solid. (ES, m/z): [M+H]⁺ 479; ¹H NMR (400 MHz, DMSO-d₆) δ 0.90 (3H, t, J=7.3 Hz), 2.04 (2H, br. s), 2.12 (3H, s), 2.39 (3H, s), 4.28 (3H, s), 6.99 (1H, d, J=8.3 Hz), 7.67 (1H, d, J=8.3 Hz), 8.02 (1H, s), 8.04 (1H, d, J=8.7 Hz), 8.45 (1H, dd, J=8.7, 2.3 Hz), 8.96 (1H, d, J=0.9 Hz), 10.09 (1H, s).

### Example 170—Synthesis of N-(6-Methoxy-1-Methyl-1H-Indazol-7-yl)-1-(5-Methoxy-2-(Trifluoromethyl) Pyridin-4-yl)-1H-Pyrazole-4-Sulfonamide (I-155)

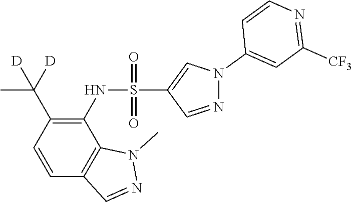 [SMILES: [2H]C([2H])(C)c1ccc2cnn(C)c2c1NS(=O)(=O)c1cnn(-c2ccnc(C(F)(F)F)c2)c1]

The title compound was obtained using general procedure A as a white solid (7 mg, 1%). (ES, m/z): [M+H]⁺ 453; ¹H NMR (400 MHz, DMSO-d₆) δ 0.84 (s, 3H), 4.26 (s, 3H), 4.24 (s, 3H), 6.99 (d, J=8.4 Hz, 1H), 7.64 (d, J=8.4 Hz, 1H), 7.99 (s, 1H), 8.15 (s, 1H), 8.21-8.38 (m, 1H), 8.47 (d, J=1.6 Hz, 1H), 8.88 (d, J=5.6 Hz, 1H), 9.38 (s, 1H), 10.17 (br s, 1H).

### Example 171—Synthesis of N-(1-Methyl-1H-Pyrazolo[4,3-C]Pyridin-7-yl)-1-(2-(Trifluoromethyl)Pyridin-4-yl)-1H-Pyrazole-4-Sulfonamide (I-156)

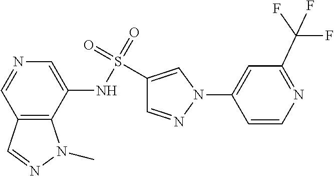 [SMILES: Cn1ncc2cncc(NS(=O)(=O)c3cnn(-c4ccnc(C(F)(F)F)c4)c3)c21]

The title compound was obtained using general procedure B as a white solid (20 mg, 9%). (ES, m/z): [M+H]⁺ 415; ¹H NMR (400 MHz, DMSO-d₆): δ 4.40 (s, 3H), 7.78 (s, 1H), 8.15 (d, J=4.0 Hz, 1H), 8.24 (dd, J=5.5, 2.1 Hz, 1H), 8.36 (s, 1H), 8.44 (d, J=2.1 Hz, 1H), 8.78 (s, 1H), 8.85 (d, J=5.5 Hz, 1H), 9.37 (s, 1H).

### Example 172—Synthesis of N-(3-Bromo-1-Methyl-1H-Indazol-7-yl)-1-(2-(Trifluoromethyl)Pyridin-4-yl)-1H-Pyrazole-4-Sulfonamide

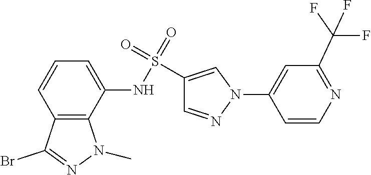 [SMILES: Cn1nc(Br)c2cccc(NS(=O)(=O)c3cnn(-c4ccnc(C(F)(F)F)c4)c3)c21]

The title compound was obtained using general procedure A as a white solid (125 mg, 70%). (ES, m/z): [M+H]⁺ 501.

### Example 173—Synthesis of N-(1,3-dimethyl-1H-Indazol-7-yl)-1-(2-(Trifluoromethyl)Pyridin-4-yl)-1H-Pyrazole-4-Sulfonamide (I-157)

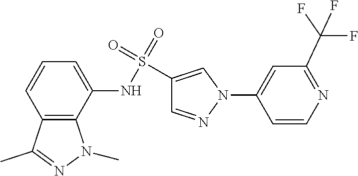 [SMILES: Cc1nn(C)c2c(NS(=O)(=O)c3cnn(-c4ccnc(C(F)(F)F)c4)c3)cccc12]

A mixture of N-(3-bromo-1-methylindazol-7-yl)-1-[2-(Trifluoromethyl)Pyridin-4-yl]pyrazole-4-sulfonamide (80 mg, 0.2 mmol, 1 eq.), 2,4,6-trimethyl-1,3,5,2,4,6-trioxatriborinane (60.10 mg, 0.480 mmol, 3 eq.), Cs₂CO₃ (155.99 mg, 0.480 mmol, 3 eq.) and Pd(dppf)Cl₂ (23.35 mg, 0.032 mmol, 0.2 eq.) in dioxane (20 mL) and H₂O (2 mL) was stirred for 2 h at 90° C. under nitrogen. The mixture was allowed to cool down to room temperature, and then poured into water and extracted with EtOAc (2×20 mL). The combined organic layers were washed with brine (20 mL), dried over anhydrous Na₂SO₄, and concentrated under reduced pressure. The residue was purified by silica gel column chromatography and eluted with PE/EtOAc (1:1). The resulting material (43 mg) was further purified by prep-HPLC with the following conditions: column: XBridge Shield RP18 OBD column, 30*150 mm, 5 μm; mobile phase A: water (10 mmol/L NH₄CO₃), mobile phase B: ACN; flow rate: 60 mL/min; gradient: 22% B to 47% B in 8 min, 47% B; wavelength: 220/254 nm, to afford the title product (8 mg, 12%) as an off-white solid. (ES, m/z): [M+H]⁺ 437; ¹H NMR (400 MHz, DMSO-d₆) δ 2.45 (s, 3H), 4.20 (s, 3H), 6.73 (d, J=7.2 Hz, 1H), 6.93 (t, J=7.6 Hz, 1H), 7.60 (s, 1H), 8.13 (s, 1H), 8.19-8.32 (m, 1H), 8.46 (d, J=2.0 Hz, 1H), 8.88 (d, J=5.5 Hz, 1H), 9.35 (s, 1H), 10.07 (s, 1H).

### Example 174—Synthesis of N-(4-bromo-1-Methyl-1H-Indazol-7-yl)-1-(2-(Trifluoromethyl)Pyridin-4-yl)-1H-Pyrazole-4-Sulfonamide

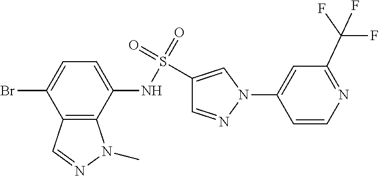 [SMILES: Cn1ncc2c(Br)ccc(NS(=O)(=O)c3cnn(-c4ccnc(C(F)(F)F)c4)c3)c21]

The title compound was obtained using general procedure A as a white solid (355 mg, 53%). (ES, m/z): [M+H]⁺ 501.

### Example 175—Synthesis of N-(1,4-Dimethyl-1H-Indazol-7-yl)-1-(2-(Trifluoromethyl)Pyridin-4-yl)-1H-Pyrazole-4-Sulfonamide (I-158)

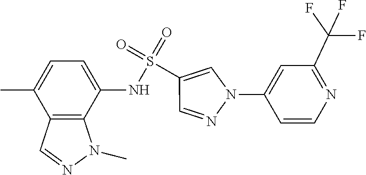 [SMILES: Cc1ccc(NS(=O)(=O)c2cnn(-c3ccnc(C(F)(F)F)c3)c2)c2c1cnn2C]

To a stirred solution of N-(4-bromo-1-methylindazol-7-yl)-1-[2-(trifluoromethyl)pyridin-4-yl]pyrazole-4-sulfonamide (200 mg, 0.4 mmol, 1.0 eq.) and trimethyl-1,3,5,2,4,6-trioxatriborinane (150 mg, 1.2 mmol, 3.0 eq.) in dioxane (5 mL) were added Pd(dppf)Cl₂·CH₂Cl₂ (33 mg, 0.04 mmol, 0.1 eq.) and Cs₂CO₃ (260 mg, 0.8 mmol, 2.0 eq.) in portions at r.t under nitrogen. The solution was stirred at 90° C. for 16 h, and then allowed to cool. The resulting mixture was diluted with water (20 mL) and extracted with EtOAc (3×50 mL). The combined organic layers were dried over anhydrous Na₂SO₄ and concentrated under reduced pressure. The crude product was purified by prep-HPLC with the following conditions: column: XBridge Prep OBD C18 column, 30*150 mm, 5 μm; mobile phase A: water (10 mmol/L NH₄CO₃), mobile phase B: ACN; flow rate: 60 mL/min; gradient: 27% B to 57% B in 7 min, 57% B; wavelength: 220 nm; Rt(min): 8.70) to afford the title compound as an off-white solid. (ES, m/z): [M+H]⁺ 437; ¹H NMR (400 MHz, DMSO-d₆) δ 2.49 (s, 3H), 4.28 (s, 3H), 6.62 (d, J=7.4 Hz, 1H), 6.75 (dd, J=7.4, 1.2 Hz, 1H), 8.11 (s, 1H), 8.16 (s, 1H), 8.28 (dd, J=5.5, 2.1 Hz, 1H), 8.48 (d, J=2.1 Hz, 1H), 8.89 (d, J=5.5 Hz, 1H), 9.37 (s, 1H), 10.01 (s, 1H).

### Example 176—Synthesis of N-(4-Methoxy-1-Methyl-1H-Indazol-7-yl)-1-(2-(Trifluoromethyl)Pyridin-4-yl)-1H-Pyrazole-4-Sulfonamide (I-159)

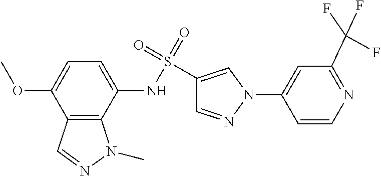 [SMILES: COc1ccc(NS(=O)(=O)c2cnn(-c3ccnc(C(F)(F)F)c3)c2)c2c1cnn2C]

The title compound was obtained using general procedure A as a white solid (20 mg, 8%). (ES, m/z): [M+H]⁺ 453.20; ¹H NMR (400 MHz, DMSO-d₆) δ 3.85 (s, 3H), 4.25 (s, 3H), 6.41 (d, J=8.0 Hz, 1H), 6.64 (d, J=8.0 Hz, 1H), 8.01 (s, 1H), 8.15 (s, 1H), 8.28 (dd, J=5.6, 2.0 Hz, 1H), 8.48 (d, J=2.0 Hz, 1H), 8.89 (d, J=5.5 Hz, 1H), 9.37 (s, 1H), 9.89 (s, 1H).

### Example 177—Synthesis of N-(4-Fluoro-1-Methyl-1H-Indazol-7-yl)-1-(2-(Trifluoromethyl)Pyridin-4-yl)-1H-Pyrazole-4-Sulfonamide (I-160)

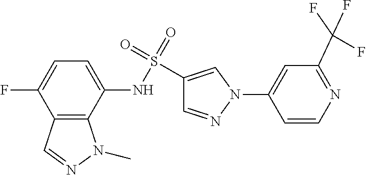 [SMILES: Cn1ncc2c(F)ccc(NS(=O)(=O)c3cnn(-c4ccnc(C(F)(F)F)c4)c3)c21]

The title compound was obtained using general procedure A as a white solid (20 mg, 8%). (ES, m/z): [M+H]⁺ 441; ¹H NMR (400 MHz, DMSO-d₆) δ 4.31 (s, 3H), 6.66-6.85 (m, 2H), 8.14-8.32 (m, 3H), 8.47 (d, J=2.0 Hz, 1H), 8.89 (d, J=5.6 Hz, 1H), 9.38 (s, 1H), 10.11 (s, 1H).

### Example 178—Synthesis of N-(1-Methyl-1H-Benzo[D][1,2,3]Triazol-7-yl)-1-(2-(Trifluoromethyl)Pyridin-4-yl)-1H-Pyrazole-4-Sulfonamide (I-161)

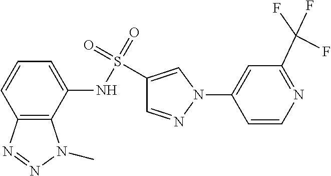 [SMILES: Cn1nnc2cccc(NS(=O)(=O)c3cnn(-c4ccnc(C(F)(F)F)c4)c3)c21]

The title compound was obtained using general procedure A as an off-white solid (8 mg, 6%). (ES, m/z): [M+H]⁺=424; ¹H NMR (400 MHz, DMSO-d₆) δ 4.50 (s, 3H), 6.91 (d, J=2.0 Hz, 1H), 7.23 (dd, J=7.6 Hz, 2.8 Hz, 1H), 7.85 (s, 1H), 8.15 (s, 1H), 8.25 (dd, J=7.2 Hz, 2.0 Hz, 1H), 8.45 (d, J=2.0 Hz, 1H), 8.87 (d, J=5.6 Hz, 1H), 9.35 (s, 1H), 10.36 (s, 1H).

### Example 179—Synthesis of 1-(6-Chloro-4-Methylpyridin-2-yl)-N-(6-Methoxy-1-Methyl-1H-Indazol-7-yl)-1H-Pyrazole-4-Sulfonamide

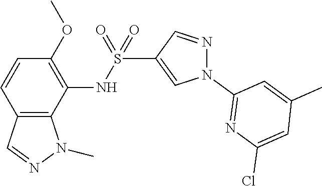 [SMILES: COc1ccc2cnn(C)c2c1NS(=O)(=O)c1cnn(-c2cc(C)cc(Cl)n2)c1]

The title compound was obtained using general procedure C as an off-white solid (120 mg, 85%). (ES, m/z): [M+H]⁺ 433.1; ¹H NMR (400 MHz, DMSO-d₆) δ 2.46 (s, 3H), 3.35 (s, 3H), 4.24 (s, 3H), 6.88 (d, J=8.9 Hz, 1H), 7.49-7.43 (m, 1H), 7.67 (s, 1H), 7.83 (t, J=1.0 Hz, 1H), 8.01-7.95 (m, 2H), 8.65 (d, J=0.8 Hz, 1H), 9.81 (s, 1H).

### Example 180—Synthesis of N-(6-Methoxy-1-Methyl-1H-Indazol-7-yl)-1-(4-Methyl-6-(4-Methylpiperazin-1-yl)Pyridin-2-yl)-1H-Pyrazole-4-Sulfonamide (I-162)

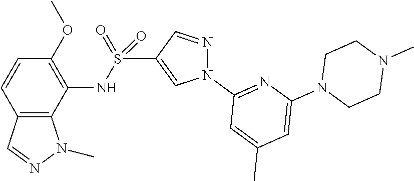 [SMILES: COc1ccc2cnn(C)c2c1NS(=O)(=O)c1cnn(-c2cc(C)cc(N3CCN(C)CC3)n2)c1]

The title compound was obtained using general procedure C as an off-white solid (33 mg, 47%). (ES, m/z): [M+H]⁺ 497.2; ¹H NMR (400 MHz, DMSO-d₆) δ 2.22 (s, 3H), 2.32 (s, 3H), 2.40 (t, J=5.1 Hz, 4H), 3.52 (t, J=5.0 Hz, 4H), 4.24 (s, 3H), 6.70 (s, 1H), 6.87 (d, J=8.9 Hz, 1H), 7.04 (s, 1H), 7.66 (d, J=8.8 Hz, 1H), 7.89 (d, J=0.8 Hz, 1H), 7.98 (s, 1H), 8.62 (d, J=0.8 Hz, 1H), 9.69 (s, 1H).

### Example 181—Synthesis of 1-(5-cyano-4-Methylpyridin-2-yl)-N-(6-Methoxy-1-Methylindazol-7-yl)Pyrazole-4-Sulfonamide (I-163)

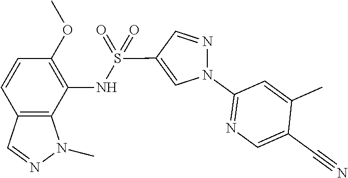 [SMILES: COc1ccc2cnn(C)c2c1NS(=O)(=O)c1cnn(-c2cc(C)c(C#N)cn2)c1]

The title compound was obtained using general procedure C as an off-white solid (57 mg, 52%). (ES, m/z): [M+H]⁺ 424.1; ¹H NMR (400 MHz, DMSO-d₆): δ 2.62 (s, 3H), 3.33 (s, 3H), 4.24 (s, 3H), 6.87 (d, J=8.8 Hz, 1H), 7.67 (d, J=8.8 Hz, 1H), 7.98 (s, 1H), 8.07-8.14 (m, 2H), 8.74 (d, J=0.8 Hz, 1H), 8.92 (s, 1H), 9.86 (s, 1H).

### Example 182—Synthesis of Methyl 6-(4-(N-(6-Methoxy-1-Methyl-1H-Indazol-7-yl)Sulfamoyl)-1H-Pyrazol-1-yl)-4-Methylnicotinate

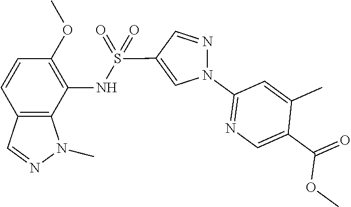 [SMILES: COC(=O)c1cnc(-n2cc(S(=O)(=O)Nc3c(OC)ccc4cnn(C)c34)cn2)cc1C]

The title compound was obtained using general procedure C as an off-white solid (277 mg, 93%). (ES, m/z): [M+H]⁺ 457.1.

### Example 183—Synthesis of 6-(4-(N-(6-Methoxy-1-Methyl-1H-Indazol-7-yl)Sulfamoyl)-1H-Pyrazol-1-yl)-4-Methylnicotinic Acid

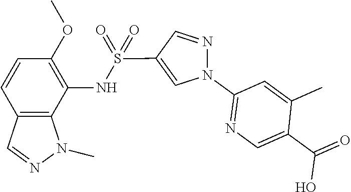 [SMILES: COc1ccc2cnn(C)c2c1NS(=O)(=O)c1cnn(-c2cc(C)c(C(=O)O)cn2)c1]

A mixture of methyl 6-(4-(N-(6-Methoxy-1-Methyl-1H-Indazol-7-yl)sulfamoyl)-1H-pyrazol-1-yl)-4-methylnicotinate (100 mg, 0.2 mmol, 1.0 eq.) and NaOH (35 mg, 0.9 mmol, 4.0 eq.) in THF (1 mL) and H₂O (1 mL) was stirred for 1 h at room temperature. The mixture was then acidified to pH 3 with HCl (aq.), and then extracted with EtOAc (2×20 mL). The combined organic layers were washed with brine (4×40 mL), dried over Na₂SO₄, and concentrated under reduced pressure to afford the title acid (90 mg, 93%) as a brown solid. (ES, m/z): [M+H]⁺ 443.0.

### Example 184—Synthesis of 1-[5-(3-Hydroxyazetidine-1-Carbonyl)-4-Methylpyridin-2-yl]-N-(6-Methoxy-1-Methylindazol-7-yl)Pyrazole-4-Sulfonamide (I-164)

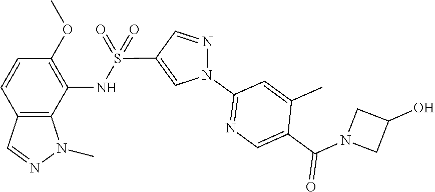 [SMILES: COc1ccc2cnn(C)c2c1NS(=O)(=O)c1cnn(-c2cc(C)c(C(=O)N3CC(O)C3)cn2)c1]

A solution of 6-(4-(N-(6-Methoxy-1-Methyl-1H-Indazol-7-yl)sulfamoyl)-1H-pyrazol-1-yl)-4-methylnicotinic acid (100 mg, 0.2 mmol, 1.0 eq.) in DMF (2 mL) was treated with DIEA (8863 mg, 0.7 mmol, 3.0 eq.) for 0.5 h at room temperature, followed by the addition of HATU (129 mg, 0.3 mmol, 1.5 eq.) and azetidin-3-ol hydrochloride (30 mg, 0.3 mmol, 1.2 eq.). The resulting mixture was stirred for 1 h at room temperature, and then poured into water and extracted with EtOAc (3×50 mL). The combined organic layers were washed with brine (4×50 mL), dried over Na₂SO₄, and concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. The crude product (130 mg) was purified by prep-HPLC (column: XBridge Prep OBD C18 column, 30*150 mm, 5 μm; flow rate: 60 mL/min; gradient: 8% B to 35% B in 8 min, 35% B; wavelength: 220 nm; Rt(min): 7.58) to afford the title compound (40 mg, 35%) as a white solid. (ES, m/z): [M+H]⁺ 498.1; ¹H NMR (400 MHz, DMSO-d₆): δ 3.30 (s, 3H), 3.33 (s, 3H), 3.75-3.85 (m, 2H), 4.10-4.20 (m, 1H), 4.24 (s, 3H), 4.22-4.32 (m, 1H), 4.45-4.55 (m, 1H), 5.80 (d, J=6.4 Hz, 1H), 6.87 (d, J=8.98 Hz, 1H), 7.67 (d, J=8.8 Hz, 1H), 7.93 (s, 1H), 7.96-8.03 (m, 2H), 8.41 (s, 1H), 8.70 (d, J=0.8 Hz, 1H), 9.78 (s, 1H).

### Example 185—Synthesis of N-(6-Methoxy-1-Methyl-1H-Indazol-7-yl)-1-(4-Methyl-5-(4-Methylpiperazine-1-carbonyl)Pyridin-2-yl)-1H-Pyrazole-4-Sulfonamide (I-165)

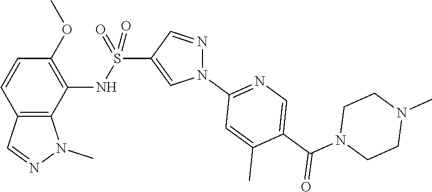 [SMILES: COc1ccc2cnn(C)c2c1NS(=O)(=O)c1cnn(-c2cc(C)c(C(=O)N3CCN(C)CC3)cn2)c1]

A solution of 6-(4-(N-(6-Methoxy-1-Methyl-1H-Indazol-7-yl)sulfamoyl)-1H-pyrazol-1-yl)-4-methylnicotinic acid (10 mg, 0.02 mmol, 1.0 eq.) and DIEA (9 mg, 0.07 mmol, 3.0 eq.) in DMF (1 mL) was stirred for 30 min at room temperature. To the above mixture was added piperazine, 1-methyl- (27 mg, 0.3, 1.2 eq.) and HATU (13 mg, 0.04 mmol, 1.5 eq.). The resulting mixture was stirred for 1 h at room temperature, and then was poured into water and extracted with EtOAc (2×20 mL). The combined organic layers were washed with brine (4×40 mL), dried over Na₂SO₄, and concentrated under reduced pressure. The crude product was purified by prep-HPLC with the following conditions: column: XBridge Shield RP18 OBD column, 30*150 mm, m; mobile phase A: water (10 mmol/L NH₄CO₃+0.1% NH₃·H₂O), mobile phase B: ACN; flow rate: 60 mL/min; gradient: 17% B to 37% B in 8 min, 37% B; wavelength: 220/254 nm; Rt(min): 7.82) to afford title compound (45 mg, 38%) as a white solid. (ES, m/z): [M+H]⁺ 525.2; ¹H NMR (400 MHz, DMSO-d₆): δ 2.20 (s, 3H), 2.25 (m, 2H), 2.37-2.35 (s, 3H), 2.40 (m, 2H), 3.22 (t, J=5.2 Hz, 2H), 3.34 (s, 3H), 3.68 (m, 2H), 4.24 (s, 3H), 6.88 (d, J=8.9 Hz, 1H), 7.67 (d, J=8.8 Hz, 1H), 7.93 (d, J=0.7 Hz, 1H), 7.98 (s, 1H), 8.00 (d, J=0.8 Hz, 1H), 8.32 (s, 1H), 8.69 (d, J=0.8 Hz, 1H), 9.79 (s, 1H).

### Example 186—Synthesis of N-(5-Fluoro-1-Methyl-1H-Indazol-7-yl)-1-(2-(Trifluoromethyl)Pyridin-4-yl)-1H-Pyrazole-4-Sulfonamide

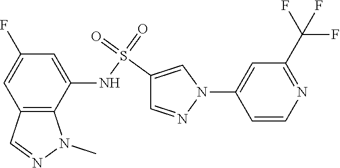 [SMILES: Cn1ncc2cc(F)cc(NS(=O)(=O)c3cnn(-c4ccnc(C(F)(F)F)c4)c3)c21]

The title compound was obtained using general procedure A as a white solid (168 mg, 4%). (ES, m/z): [M+H]⁺ 441; ¹H NMR (400 MHz, DMSO-d₆) δ 4.26 (s, 3H), 6.69 (dd, J=9.6, 2.4 Hz, 1H), 8.05 (s, 1H), 8.19 (s, 1H), 8.24-8.32 (m, 2H), 8.47 (d, J=1.6 Hz, 1H), 8.88 (d, J=5.6 Hz, 1H), 9.43 (s, 1H), 10.35 (s, 1H).

### Example 187—Synthesis of N-(3-Methoxy-1-Methyl-1H-Indazol-7-yl)-1-(2-(Trifluoromethyl)Pyridin-4-yl)-1H-Pyrazole-4-Sulfonamide (I-167)

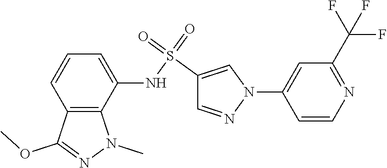 [SMILES: COc1nn(C)c2c(NS(=O)(=O)c3cnn(-c4ccnc(C(F)(F)F)c4)c3)cccc12]

The title compound was obtained using general procedure A as an off-white solid (22 mg, 17%). (ES, m/z): [M+H]⁺ 453; ¹H NMR (400 MHz, DMSO-d₆) δ 3.99 (s, 3H), 4.09 (s, 3H), 6.75 (d, J=7.3 Hz, 1H), 6.88 (t, J=7.7 Hz, 1H), 7.51 (d, J=8.0 Hz, 1H), 8.15 (s, 1H), 8.21-8.37 (m, 1H), 8.47 (s, 1H), 8.88 (d, J=5.5 Hz, 1H), 9.38 (s, 1H), 10.08 (s, 1H).

### Example 188—Synthesis of 6-(4-(N-(6-Methoxy-1-Methyl-1H-Indazol-7-yl)Sulfamoyl)-1H-Pyrazol-1-yl)-N,N,4-Trimethylnicotinamide (I-168)

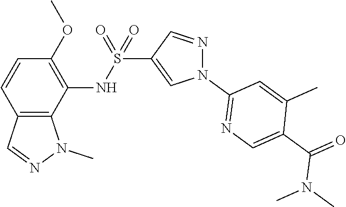 [SMILES: COc1ccc2cnn(C)c2c1NS(=O)(=O)c1cnn(-c2cc(C)c(C(=O)N(C)C)cn2)c1]

A solution of 6-(4-(N-(6-Methoxy-1-Methyl-1H-Indazol-7-yl)sulfamoyl)-1H-pyrazol-1-yl)-4-methylnicotinic acid (100 mg, 0.2 mmol, 1.0 eq.) in DMF (1 mL) was treated with DIEA (88 mg, 0.7 mmol, 3.0 eq.) for 0.5 h at room temperature followed by the addition of HATU (129 mg, 0.3 mmol, 1.5 eq.). The resulting mixture was stirred for 1 h at room temperature, then extracted with EtOAc (2×20 mL). The combined organic layers were washed with brine (4×40 mL), dried over Na₂SO₄, and concentrated under reduced pressure. The crude product was purified by prep-HPLC with the following conditions: column: XBridge Prep OBD C18 column, 30*150 mm, 5 μm; mobile phase A: water (10 mmol/L NH₄CO₃), mobile phase B: ACN; flow rate: 60 mL/min; gradient: 19% B to 40% B in 8 min, 40% B; wavelength: 254 nm; Rt(min): 7.9) to afford the title compound (41 mg, 39%) as a white solid. (ES, m/z): [M+H]⁺ 470.1; ¹H NMR (400 MHz, DMSO-d₆): δ 2.39-2.31 (m, 3H), 2.84 (s, 3H), 3.04 (s, 3H), 3.34 (s, 3H), 4.24 (s, 3H), 6.88 (d, J=8.9 Hz, 1H), 7.67 (d, J=8.8 Hz, 1H), 8.03-7.91 (m, 3H), 8.33 (s, 1H), 8.69 (d, J=0.7 Hz, 1H), 9.75 (s, 1H).

### Example 189—Synthesis of N-(1-Methylindazol-6-yl)-1-[4-(Trifluoromethyl)Pyridin-2-yl]Pyrazole-4-Sulfonamide (I-169)

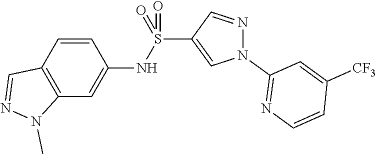 [SMILES: Cn1ncc2ccc(NS(=O)(=O)c3cnn(-c4cc(C(F)(F)F)ccn4)c3)cc21]

The title compound was obtained using general procedure A as a light pink solid (48 mg, 17%). (ES, m/z): [M+H]⁺ 470.1; H NMR (400 MHz, DMSO-d₆) δ 3.96 (s, 3H), 6.95 (dd, J=8.6, 1.8 Hz, 1H), 7.37 (m, 1H), 7.64 (d, J=8.6 Hz, 1H), 7.84 (dd, J=5.4, 1.5 Hz, 1H), 7.94 (d, J=1.0 Hz, 1H), 8.10 (s, 1H), 8.18 (m, 1H), 8.78 (d, J=5.1 Hz, 1H), 8.98 (d, J=0.7 Hz, 1H), 10.52 (s, 1H).

### Example 190—Synthesis of 1-(5-Formyl-4-Methylpyridin-2-yl)-N-(6-Methoxy-1-Methylindazol-7-yl)Pyrazole-4-Sulfonamide

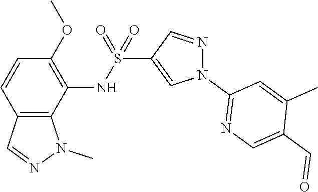 [SMILES: COc1ccc2cnn(C)c2c1NS(=O)(=O)c1cnn(-c2cc(C)c(C=O)cn2)c1]

The title compound was obtained using general procedure C as a light pink solid (80 mg, 52%). (ES, m/z): [M+H]⁺ 427.1.

### Example 191—Synthesis of 1-[5-(Hydroxymethyl)-4-Methylpyridin-2-yl]-N-(6-Methoxy-1-Methylindazol-7-yl)Pyrazole-4-Sulfonamide (I-170)

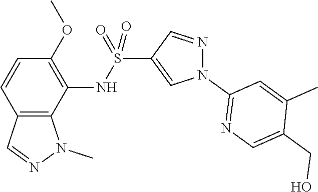 [SMILES: COc1ccc2cnn(C)c2c1NS(=O)(=O)c1cnn(-c2cc(C)c(CO)cn2)c1]

A mixture of 1-(5-formyl-4-methylpyridin-2-yl)-N-(6-methoxy-1-methylindazol-7-yl) pyrazole-4-sulfonamide (50 mg, 0.1 mmol, 1.0 eq.) and NaBH₄ (4 mg, 0.1 mmol, 1.0 eq.) in THF (3 mL) was stirred for 2 h at room temperature. The resulting mixture was then quenched with water and extracted with EtOAc (3×40 mL). The combined organic layers were washed with brine (4×40 mL), dried over Na₂SO₄, and concentrated under reduced pressure. The crude product was purified by prep-HPLC with the following conditions: column: XBridge Prep OBD C18 column, 30*150 mm, 5 μm; flow rate: 60 mL/min; gradient: 10% B to 40% B in 8 min, 40% B; wavelength: 220 nm; Rt(min): 7.58) to afford the title compound (13 mg, 26%) as a white solid. (ES, m/z): [M+H]⁺ 427.1; ¹H NMR (400 MHz, DMSO-d₆): δ 9.76 (s, 1H), 8.68 (d, J=0.7 Hz, 1H), 8.37 (s, 1H), 8.00-7.93 (m, 2H), 7.82 (s, 1H), 7.67 (d, J=8.8 Hz, 1H), 6.86 (d, J=8.9 Hz, 1H), 5.31 (t, J=5.4 Hz, 1H), 4.58 (d, J=5.3 Hz, 2H), 4.24 (s, 3H), 3.03 (s, 3H), 2.42 (s, 3H).

### Example 192—Synthesis of N-(3-Fluoro-1-Methyl-1H-Indazol-7-yl)-1-(2-(Trifluoromethyl)Pyridin-4-yl)-1H-Pyrazole-4-Sulfonamide (I-171)

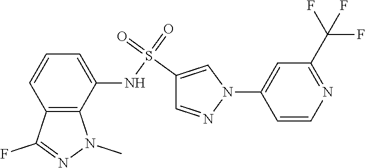 [SMILES: Cn1nc(F)c2cccc(NS(=O)(=O)c3cnn(-c4ccnc(C(F)(F)F)c4)c3)c21]

The title compound was obtained using general procedure C as a light pink solid (49 mg, 36%). (ES, m/z): [M+H]⁺ 441. ¹H NMR (400 MHz, DMSO-d₆): δ 4.16 (s, 3H), 6.84 (d, J=7.4 Hz, 1H), 7.04 (t, J=7.6 Hz, 1H), 7.63 (s, 1H), 8.17 (s, 1H), 8.27 (dd, J=5.6, 2.0 Hz, 1H), 8.47 (d, J=2.0 Hz, 1H), 8.88 (d, J=5.6 Hz, 1H), 9.38 (s, 1H), 10.20 (s, 1H).

### Example 193—Synthesis of N-(6-Methoxy-1-Methyl-1H-Indazol-7-yl)-1-(4-Methyl-5-(4-Methylpiperazin-1-yl)Pyridin-2-yl)-1H-Pyrazole-4-Sulfonamide (I-172)

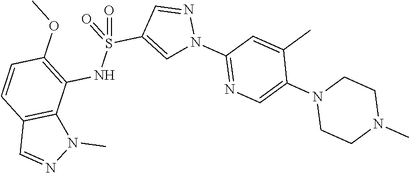 [SMILES: COc1ccc2cnn(C)c2c1NS(=O)(=O)c1cnn(-c2cc(C)c(N3CCN(C)CC3)cn2)c1]

The title compound was obtained using general procedure D as a light pink solid (12 mg, 7%). (ES, m/z): [M+H]⁺ 497.2; ¹H NMR (400 MHz, DMSO-d₆): δ 2.22 (s, 3H), 2.32 (s, 3H), 2.40 (t, J=5.1 Hz, 4H), 3.52 (t, J=5.0 Hz, 4H), 4.24 (s, 3H), 6.70 (s, 1H), 6.87 (d, J=8.9 Hz, 1H), 7.04 (s, 1H), 7.66 (d, J=8.8 Hz, 1H), 7.89 (d, J=0.8 Hz, 1H), 7.98 (s, 1H), 8.62 (d, J=0.8 Hz, 1H), 9.69 (s, 1H).

### Example 194—Synthesis of N-(6-Ethyl-1-methyl-1H-Pyrazolo[4,3-C]Pyridin-4-yl)-1-(2-(Trifluoromethyl)Pyridin-4-yl)-1H-Pyrazole-4-Sulfonamide (I-173)

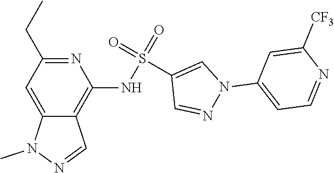 [SMILES: CCc1cc2c(cnn2C)c(NS(=O)(=O)c2cnn(-c3ccnc(C(F)(F)F)c3)c2)n1]

The title compound was obtained using general procedure A as a light pink solid (6.2 mg, 3.10%). (ES, m/z): [M+H]⁺=452, ¹H NMR (400 MHz, DMSO-d₆) δ 1.24 (t, J=7.2 Hz, 3H), 2.78 (q, J=6.8 Hz, 2H), 3.98 (s, 3H), 7.27 (s, 1H), 8.21 (t, J=6.8 Hz, 2H), 8.41 (s, 1H), 8.83 (d, J=10 Hz, 2H), 9.32 (s, 1H).

### Example 195—Synthesis of N-(1-Methyl-1H-Benzo[D][1,2,3]Triazol-7-yl)-1-(4-(Trifluoromethyl)Pyridin-2-yl)-1H-Pyrazole-4-Sulfonamide (I-174)

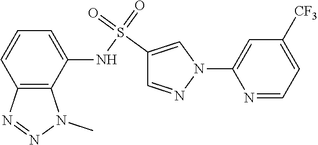 [SMILES: Cn1nnc2cccc(NS(=O)(=O)c3cnn(-c4cc(C(F)(F)F)ccn4)c3)c21]

The title compound was obtained using general procedure A as a white solid (32 mg, 12%). (ES, m/z): [M+H]⁺ 424; ¹H NMR (400 MHz, DMSO-d₆) δ 4.49 (s, 3H), 6.89 (dd, J=7.6 Hz, 0.8 Hz, 1H), 7.24 (t, J=8.0 Hz, 1H), 7.93-7.88 (m, 2H), 8.10 (s, 1H), 8.19 (s, 1H), 8.13 (d, J=5.2 Hz, 1H), 8.70 (d, J=6.4 Hz, 1H), 10.32 (s, 1H).

### Example 196—Synthesis of N-(1,5-Dimethylindazol-6-yl)-1-[4-(Trifluoromethyl)Pyridin-2-yl]Pyrazole-4-Sulfonamide (I-175)

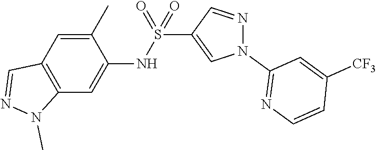 [SMILES: Cc1cc2cnn(C)c2cc1NS(=O)(=O)c1cnn(-c2cc(C(F)(F)F)ccn2)c1]

The title compound was obtained using general procedure A as a white solid (32 mg, 12%). (ES, m/z): [M+H]⁺ 437; ¹H NMR (400 MHz, DMSO-d₆) δ 2.19 (d, J=0.9 Hz, 3H), 3.93 (s, 3H), 7.37 (s, 1H), 7.53 (m, 1H), 7.86 (dt, J=5.0, 1.0 Hz, 1H), 7.91 (d, J=1.0 Hz, 1H), 8.14 (dd, J=10.1, 1.3 Hz, 2H), 8.79 (d, J=5.1 Hz, 1H), 8.86 (d, J=0.7 Hz, 1H), 9.84 (s, 1H).

### Example 197—Synthesis of N-(2,5-Dimethylindazol-6-yl)-1-[4-(Trifluoromethyl)Pyridin-2-yl]Pyrazole-4-Sulfonamide (I-176)

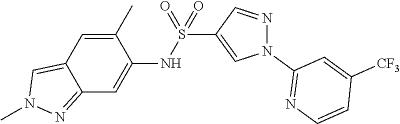 [SMILES: Cc1cc2cn(C)nc2cc1NS(=O)(=O)c1cnn(-c2cc(C(F)(F)F)ccn2)c1]

The title compound was obtained using general procedure A as a white solid (33 mg, 12%). (ES, m/z): [M+H]⁺ 437; ¹H NMR (400 MHz, DMSO-d₆) δ 2.24 (d, J=1.0 Hz, 3H), 4.08 (s, 3H), 7.22 (s, 1H), 7.48 (s, 1H), 7.86 (s, 1H), 8.10 (d, J=8.0 Hz, 1H), 8.17 (d, J=7.6 Hz, 2H), 8.79 (d, J=5.2 Hz, 1H), 8.86 (s, 1H), 9.66 (s, 1H).

### Example 198—Synthesis of 1-(4-(1,1-Difluoroethyl)Pyridin-2-yl)-N-(6-Methoxy-1-Methyl-1H-Indazol-7-yl)-1H-Pyrazole-4-Sulfonamide (I-91)

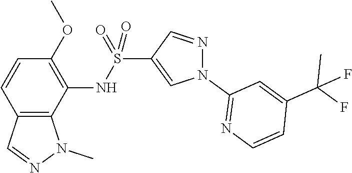 [SMILES: COc1ccc2cnn(C)c2c1NS(=O)(=O)c1cnn(-c2cc(C(C)(F)F)ccn2)c1]

The title compound was obtained using general procedure A as an off-white solid (6 mg, 4%). (ES, m/z): [M+H]⁺ 449; ¹H NMR (400 MHz, CDCl₃) δ 1.96 (t, J=18.3 Hz, 3H), 3.42 (s, 3H), 4.42 (s, 3H), 6.49 (s, 1H), 6.68 (d, J=8.9 Hz, 1H), 7.40 (dd, J=1.6, 5.2 Hz, 1H), 7.62 (d, J=8.8 Hz, 1H), 7.73 (s, 1H), 7.95 (s, 1H), 8.08 (s, 1H), 8.50 (d, J=5.1 Hz, 1H), 8.73 (s, 1H).

### Example 199—Synthesis of 1-(4-(3-Fluorooxetan-3-yl)Pyridin-2-yl)-N-(6-Methoxy-1-Methyl-1H-Indazol-7-yl)-1H-Pyrazole-4-Sulfonamide (I-69)

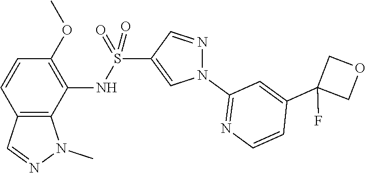 [SMILES: COc1ccc2cnn(C)c2c1NS(=O)(=O)c1cnn(-c2cc(C3(F)COC3)ccn2)c1]

The title compound was obtained using general procedure B as a white solid (5 mg, 2%). (ES, m/z): [M+H]⁺ 459; ¹H NMR (400 MHz, DMSO-d₆) δ 3.34 (s, 3H), 4.25 (s, 3H), 4.91 (d, J=9.6 Hz, 1H), 4.97 (d, J=9.2 Hz, 1H), 5.01 (d, J=9.2 Hz, 1H), 5.06 (d, J=9.6 Hz, 1H), 6.87 (d, J=8.8 Hz, 1H), 7.65 (d, J=8.8 Hz, 1H), 7.69 (dd, J=5.2, 1.2 Hz, 1H), 7.97 (s, 1H), 8.02 (s, 1H), 8.08 (s, 1H), 8.64 (d, J=5.2 Hz, 1H), 8.74 (s, 1H), 9.84 (s, 1H).

### Example 200—Synthesis of N-(6-(Methoxy-D₃)-1-Methyl-1H-Indazol-7-yl)-1-(4-(Trifluoromethyl)Pyridin-2-yl)-1H-Pyrazole-4-Sulfonamide (I-70)

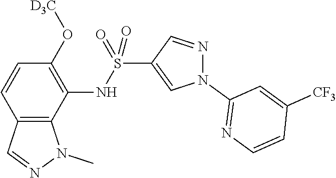 [SMILES: [2H]C([2H])([2H])Oc1ccc2cnn(C)c2c1NS(=O)(=O)c1cnn(-c2cc(C(F)(F)F)ccn2)c1]

The title compound was obtained using general procedure A as an off-white solid (31 mg, 13%). (ES, m/z): [M+H]⁺ 456; ¹H NMR (400 MHz, DMSO-d₆) δ 4.24 (s, 3H), 6.87 (d, J=8.9 Hz, 1H), 7.68 (d, J=8.8 Hz, 1H), 7.88 (d, J=5.0 Hz, 1H), 7.99 (s, 1H), 8.08 (d, J=0.7 Hz, 1H), 8.20 (s, 1H), 8.75-8.86 (m, 2H), 9.87 (s, 1H).

### Example 201—Synthesis of N-(1-Methyl-6-(Methyl-D₃)-1H-Indazol-7-yl)-1-(4-(Trifluoromethyl)Pyridin-2-yl)-1H-Pyrazole-4-Sulfonamide (I-71)

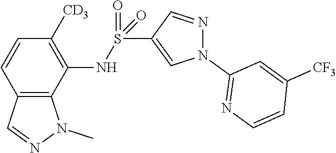 [SMILES: [2H]C([2H])([2H])c1ccc2cnn(C)c2c1NS(=O)(=O)c1cnn(-c2cc(C(F)(F)F)ccn2)c1]

The title compound was obtained using general procedure A as a white solid (36 mg, 29%). (ES, m/z): [M+H]⁺ 440; ¹H NMR (400 MHz, DMSO-d₆) δ 4.25 (s, 3H), 6.91 (d, J=8.2 Hz, 1H), 7.60 (d, J=8.2 Hz, 1H), 7.89 (dd, J=1.6, 5.1 Hz, 1H), 8.00 (s, 1H), 8.09 (s, 1H), 8.20 (s, 1H), 8.82 (d, J=5.8 Hz, 2H), 10.12 (s, 1H).

### Example 202—Synthesis of N-(6-Methoxy-1-(Methyl-D₃)-1H-Indazol-7-yl)-1-(4-(Trifluoromethyl)Pyridin-2-yl)-1H-Pyrazole-4-Sulfonamide (I-72)

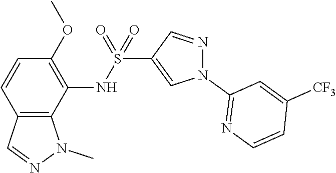 [SMILES: COc1ccc2cnn(C)c2c1NS(=O)(=O)c1cnn(-c2cc(C(F)(F)F)ccn2)c1]

The title compound was obtained using general procedure A as a white solid (63 mg, 25%). (ES, m/z): [M+H]⁺ 456; ¹H NMR (400 MHz, DMSO-d₆) δ 3.35 (s, 3H), 6.88 (d, J=8.9 Hz, 1H), 7.68 (d, J=8.8 Hz, 1H), 7.88 (dd, J=5.2, 1.6 Hz, 1H), 7.98 (s, 1H), 8.08 (s, 1H), 8.19 (s, 1H), 8.80 (m, 2H), 9.85 (s, 1H).

### Example 203—Synthesis of 1-Methyl-N-(1-(4-(Trifluoromethyl)Pyridin-2-yl)-1H-Pyrazol-4-yl)-1H-Indazole-7-Sulfonamide (II-21)

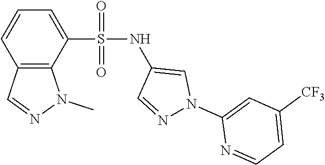 [SMILES: Cn1ncc2cccc(S(=O)(=O)Nc3cnn(-c4cc(C(F)(F)F)ccn4)c3)c21]

The title compound was obtained using general procedure C as a white solid (85 mg, 19%). (ES, m/z): [M+H]⁺ 423; ¹H NMR (400 MHz, DMSO-d₆) δ 4.57 (s, 3H), 6.37 (s, 1H), 7.16 (t, J=7.7 Hz, 1H), 7.39 (d, J=5.1 Hz, 1H), 7.52 (s, 1H), 7.98 (dd, J=7.7, 3.7 Hz, 2H), 8.14 (s, 2H), 8.45 (s, 1H), 8.52 (d, J=5.1 Hz, 1H).

### Example 204—Synthesis of 1-Methyl-N-(1-(4-(Trifluoromethyl)Pyridin-2-yl)-1H-Pyrazol-4-yl)-1H-Indazole-7-Sulfonamide (I-178)

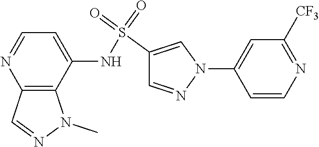 [SMILES: Cn1ncc2nccc(NS(=O)(=O)c3cnn(-c4ccnc(C(F)(F)F)c4)c3)c21]

The title compound was obtained using general procedure B as a grey solid (42 mg, 10%). (ES, m/z): [M+H]⁺ 424; ¹H NMR (400 MHz, DMSO-d₆): δ 4.38 (s, 3H), 7.07 (d, J=6.8 Hz, 1H), 7.99 (s, 1H), 8.03 (d, J=6.8 Hz, 1H), 8.25 (d, J=4.0 Hz, 2H), 8.45 (s, 1H), 8.85 (d, J=5.6 Hz, 1H), 9.49 (s, 1H), 13.38 (s, 1H).

### Example 205—Synthesis of N-(1-Methyl-1H-Pyrazolo[3,4-C]Pyridin-7-yl)-1-(2-(Trifluoromethyl)Pyridin-4-yl)-1H-Pyrazole-4-Sulfonamide

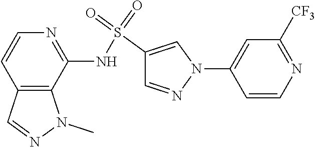 [SMILES: Cn1ncc2ccnc(NS(=O)(=O)c3cnn(-c4ccnc(C(F)(F)F)c4)c3)c21]

The title compound was obtained using general procedure A as a white solid (8 mg, 2%). (ES, m/z): [M+H]⁺ 424; ¹H NMR (400 MHz, DMSO-d₆) δ 4.33 (s, 3H), 7.04 (d, J=6.9 Hz, 1H), 7.37 (d, J=6.9 Hz, 1H), 8.05 (s, 1H), 8.23 (dd, J=5.6, 2.1 Hz, 1H), 8.43-8.38 (m, 2H), 8.87 (d, J=5.5 Hz, 1H), 9.53 (s, 1H), 11.86 (s, 1H).

### Example 206—Synthesis of N-[2-(Dimethylamino)Ethyl]-N-(6-Methoxy-1-Methylindazol-7-yl)-1-[2-(Trifluoromethyl)Pyridin-4-yl]Pyrazole-4-Sulfonamide (I-180)

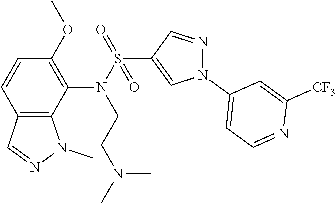 [SMILES: COc1ccc2cnn(C)c2c1N(CCN(C)C)S(=O)(=O)c1cnn(-c2ccnc(C(F)(F)F)c2)c1]

A solution of N-(6-methoxy-1-methylindazol-7-yl)-1-[2-(Trifluoromethyl)Pyridin-4-yl]pyrazole-4-sulfonamide (80 mg, 0.2 mmol, 1 eq.), (2-bromoethyl)dimethylamine hydrobromide (82 mg, 0.4 mmol, 2 eq.) and Cs₂CO₃ (170 mg, 0.5 mmol, 3 eq.) in DMF (3 mL) was stirred for 2 h at room temperature under nitrogen. The resulting mixture was diluted with water (10 mL) and extracted with EtOAc (3×10 mL). The combined organic layers were washed with brine (2×10 mL), dried over Na₂SO₄, and concentrated under reduced pressure. The crude product (80 mg) was purified by prep-HPLC with the following conditions: column: XBridge Prep Phenyl OBD column, 19*250 mm, 5 μm; mobile phase A: water (10 mmol/L NH₄CO₃+0.1% NH₃·H₂O), mobile phase B: ACN; flow rate: 25 mL/min; gradient: 62% B to 77% B in 8 min, 77% B; wavelength: 220/254 nm; Rt(min): 4.78) to afford the title compound (7.5 mg, 8%) as an off-white solid. (ES, m/z): [M+H]⁺ 524; ¹H NMR (400 MHz, DMSO-d₆) δ 2.01 (s, 6H), 2.21-2.32 (m, 2H), 3.35 (s, 3H), 3.43-3.51 (m, 1H), 3.91-4.17 (m, 1H), 4.30 (s, 3H), 6.92 (d, J=8.8 Hz, 1H), 7.43 (d, J=8.8 Hz, 1H), 7.98 (m, 1H), 8.24 (s, 1H), 8.31 (dd, J=5.2, 2.0 Hz, 1H), 8.53 (d, J=6.0 Hz, 1H), 8.91 (d, J=5.6 Hz, 1H), 9.53 (s, 1H).

### Example 207—Synthesis of N-[3-(Dimethylamino)Propyl]-N-(6-Methoxy-1-Methylindazol-7-yl)-1-[2-(Trifluoromethyl)Pyridin-4-yl]Pyrazole-4-Sulfonamide (I-181)

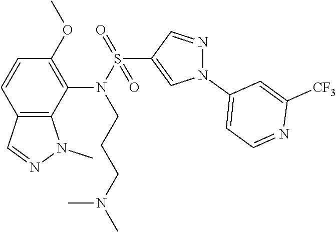 [SMILES: COc1ccc2cnn(C)c2c1N(CCCN(C)C)S(=O)(=O)c1cnn(-c2ccnc(C(F)(F)F)c2)c1]

A solution of N-(6-methoxy-1-methylindazol-7-yl)-1-[2-(Trifluoromethyl)Pyridin-4-yl]pyrazole-4-sulfonamide (80 mg, 0.2 mmol, 1.0 eq.) and (3-bromopropyl)dimethylamine hydrobromide (87 mg, 0.4 mmol, 2.0 eq.), Cs₂CO₃ (173 mg, 0.5 mmol, 3.0 eq.) in DMF (3 mL) was stirred for 2 h at room temperature under a nitrogen atmosphere. The resulting mixture was diluted with water (10 mL) and extracted with EtOAc (3×10 mL). The combined organic layers were washed with brine (2×10 mL), dried over Na₂SO₄, and concentrated under reduced pressure. The crude product (100 mg) was purified by prep-HPLC with the following conditions: column: XBridge Prep Phenyl OBD column, 19*250 mm, 5 μm; mobile phase A: water (10 mmol/L NH₄CO₃+0.1% NH₃·H₂O), mobile phase B: ACN; flow rate: 25 mL/min; gradient: 62% B to 77% B in 8 min, 77% B; wavelength: 220/254 nm; Rt(min): 4.78) to afford the title compound (24 mg, 25%) as an off-white solid. (ES, m/z): [M+H]⁺ 538; ¹H NMR (400 MHz, DMSO-d₆) δ 1.31-1.59 (m, 1H), 1.60-1.87 (m, 1H), 1.99 (s, 6H), 2.10-2.21 (m, 2H), 3.38 (s, 3H), 3.40-3.58 (m, 1H), 3.88-3.977 (m, 1H), 4.25 (s, 3H), 6.95 (d, J=8.8 Hz, 1H), 7.75 (d, J=8.8 Hz, 1H), 8.02 (m, 1H), 8.20 (s, 1H), 8.20-8.43 (m, 1H), 8.54 (d, J=2.0 Hz, 1H), 8.91 (d, J=5.6 Hz, 1H), 9.54 (s, 1H).

### Example 208—Synthesis of N-(1-Methylindol-7-yl)-1-[4-(Trifluoro-Methyl)Pyridin-2-yl]Pyrazole-4-Sulfonamide (I-182)

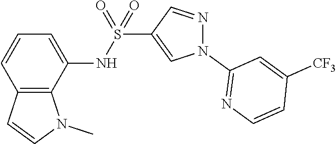 [SMILES: Cn1ccc2cccc(NS(=O)(=O)c3cnn(-c4cc(C(F)(F)F)ccn4)c3)c21]

The title compound was obtained using general procedure A as a pink solid (42 mg, 27%). (ES, m/z): [M+H]⁺ 422; ¹H NMR (400 MHz, DMSO-d₆) δ 4.08 (s, 3H). 6.44-6.48 (m, 2H), 6.82 (t, J=7.6 Hz, 1H), 7.29 (d, J=3.2 Hz, 1H), 7.46 (dd, J=7.6 Hz, 0.4 Hz, 1H), 7.88 (d, J=5.2 Hz, 1H), 8.09 (s, 1H), 8.20 (s, 1H), 8.81 (d, J=4.8 Hz, 1H), 8.84 (s, 1H), 9.92 (s, 1H).

### Example 209—Synthesis of N-(3-Methoxy-2-Methyl-2H-Indazol-7-yl)-1-(2-(Trifluoromethyl)Pyridin-4-yl)-1H-Pyrazole-4-Sulfonamide (I-183)

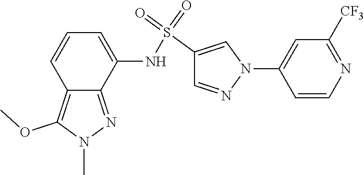 [SMILES: COc1c2cccc(NS(=O)(=O)c3cnn(-c4ccnc(C(F)(F)F)c4)c3)c2nn1C]

The title compound was obtained using general procedure A as a yellow solid (40 mg, 16%). (ES, m/z): [M+H]⁺ 453; ¹H NMR (400 MHz, DMSO-d₆) δ 3.33 (s, 3H), 3.42 (s, 3H), 6.92-7.16 (m, 2H), 7.55 (d, J=7.0 Hz, 1H), 8.15 (s, 1H), 8.17-8.29 (m, 1H), 8.26 (dd, J=5.2, 1.6 Hz, 1H), 8.43 (d, J=2.1 Hz, 1H), 8.87 (d, J=5.2 Hz, 1H), 9.33 (s, 1H).

### Example 210—Synthesis of N-(3-Methyl-1H-Indol-4-yl)-1-[4-(Trifluoromethyl)Pyridin-2-yl]Pyrazole-4-Sulfonamide (I-184)

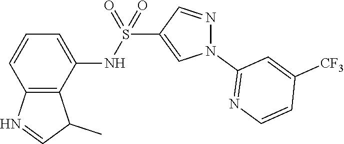 [SMILES: CC1C=Nc2cccc(NS(=O)(=O)c3cnn(-c4cc(C(F)(F)F)ccn4)c3)c21]

The title compound was obtained using general procedure A as a pink solid (48 mg, 21%). (ES, m/z): [M+H]⁺ 422, ¹H NMR (400 MHz, DMSO-d₆) δ 2.50 (s, 3H), 6.46 (d, J=7.6 Hz, 1H), 6.89 (t, J=7.6 Hz, 1H), 7.06 (s, 1H), 7.23 (d, J=8.0 Hz, 1H), 7.86 (d, J=5.2 Hz, 1H), 8.06 (s, 2H), 8.81 (d, J=8.0 Hz, 2H), 9.67 (s, 1H), 10.88 (s, 1H).

### Example 211—Synthesis of N-(6-Ethyl-1-Methyl-1H-Pyrazolo[4,3-C]Pyridin-7-yl)-1-(2-(Trifluoromethyl)Pyridin-4-yl)-1H-Pyrazole-4-Sulfonamide (I-185)

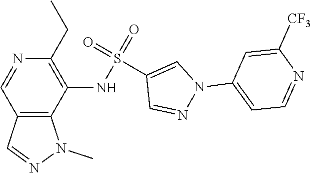 [SMILES: CCc1ncc2cnn(C)c2c1NS(=O)(=O)c1cnn(-c2ccnc(C(F)(F)F)c2)c1]

The title compound was obtained using general procedure B as a white solid (2 mg, 1%). (ES, m/z): [M+H]⁺ 452, ¹H NMR (400 MHz, DMSO-d₆) δ 0.88 (t, J=7.6 Hz, 3H), 2.33 (q, J=7.6 Hz, 2H), 4.25 (s, 3H), 8.27-8.14 (m, 3H), 8.64 (s, 1H), 8.88 (d, J=5.6 Hz, 1H), 8.94 (s, 1H), 9.39 (s, 1H).

### Example 212—Synthesis of N-(1-(Methyl-D₃)-1H-Indazol-7-yl)-1-(4-(Trifluoromethyl)Pyridin-2-yl)-1H-Pyrazole-4-Sulfonamide (I-186)

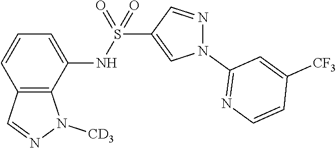 [SMILES: [2H]C([2H])([2H])n1ncc2cccc(NS(=O)(=O)c3cnn(-c4cc(C(F)(F)F)ccn4)c3)c21]

The title compound was obtained using general procedure A as a white solid (266 mg, 63%). (ES, m/z): [M+H]⁺ 426; ¹H NMR (400 MHz, DMSO-d₆) δ 6.74 (dd, J=1.0, 7.3 Hz, 1H), 6.98 (dd, J=7.3, 8.0 Hz, 1H), 7.71 (dd, J=1.0, 8.1 Hz, 1H), 7.77-7.95 (m, 1H), 8.00-8.12 (m, 2H), 8.20 (dd, J=0.8, 1.6 Hz, 1H), 8.82 (d, J=5.1 Hz, 1H), 8.88 (d, J=0.8 Hz, 1H), 10.11 (s, 1H).

### Example 213—Synthesis of N-(6-Methoxy-1-Methyl-1H-Pyrazolo[4,3-C]Pyridin-7-yl)-1-(4-(Trifluoromethyl)Pyridin-2-yl)-1H-Pyrazole-4-Sulfonamide (I-187)

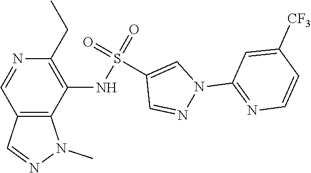 [SMILES: CCc1ncc2cnn(C)c2c1NS(=O)(=O)c1cnn(-c2cc(C(F)(F)F)ccn2)c1]

The title compound was obtained using general procedure A as a white solid (51 mg, 11%). (ES, m/z): [M+H]⁺ 454, ¹H NMR (400 MHz, DMSO-d₆) δ 3.44 (s, 3H), 4.22 (s, 3H), 7.88 (d, J=5.2 Hz, 1H), 8.11 (d, J=0.7 Hz, 1H), 8.16-8.22 (m, 1H), 8.23 (s, 1H), 8.66 (s, 1H), 8.82 (d, J=6.8 Hz, 2H), 10.00 (s, 1H).

### Example 214—Synthesis of 1-(4-(1,1-Difluoroethyl)Pyridin-2-yl)-N-(1-Methyl-1H-Pyrazolo[4,3-C]Pyridin-7-yl)-1H-Pyrazole-4-Sulfonamide (I-120)

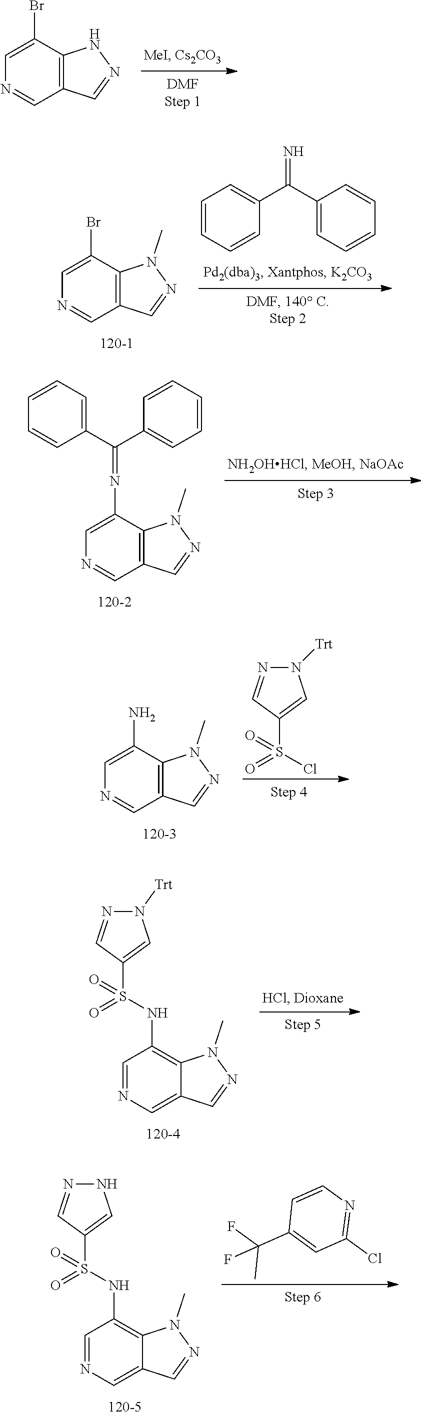

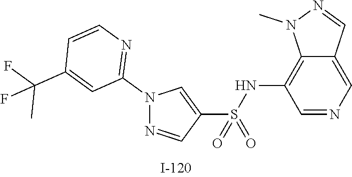 [SMILES: Cn1ncc2cncc(NS(=O)(=O)c3cnn(-c4cc(C(C)(F)F)ccn4)c3)c21]

Step 1: Synthesis of 120-1. A solution of 7-bromo-1H-pyrazolo[4,3-c]pyridine (594 mg, 3.00 mmol, 1 eq.) Cs₂CO₃ (1.95 g, 6.000 mmol, 2 eq.) and methyl iodide (638.65 mg, 4.50 mmol, 1.5 eq.) in DMF (15 mL) was stirred overnight at room temperature. After the reaction was completed, the mixture was dissolved with water (100 mL), extracted with EtOAc (4×50 mL). The organic phase was combined and washed with the brine (100 mL). Finally, the solution was dried over anhydrous Na₂SO₄ and concentrated under vacuum to give the crude product. The mixture was purified by reversed-phase flash chromatography with the following conditions: column: C18 silica gel; mobile phase: ACN in water (10 mmol/L NH₄CO₃), 0% to 100% gradient in 10 min; detector: UV 254 nm, to give 120-1 (165 mg, 25.94%) as a yellow solid.

Step 2: Synthesis of 120-2. A solution of 120-1 (548.6 mg, 2.59 mmol, 1 eq.), Pd₂(dba)₃ (473.82 mg, 0.52 mmol, 0.2 eq.), Xantphos (299.40 mg, 0.52 mmol, 0.2 eq.) and K₂CO₃ (1072.66 mg, 7.76 mmol, 3 eq.) in DMF (26 mL) was stirred overnight at 140° C. under a nitrogen atmosphere. After the reaction was completed, the mixture was added to water (150 mL) and extracted with EtOAc (4×100 mL). The organic phase was combined and washed with the brine (150 mL), dried over anhydrous Na₂SO₄, and concentrated to give the crude product. The residue was purified by reversed-phase flash chromatography with the following conditions: column: C18 silica gel; mobile phase: ACN in water (10 mmol/L NH4CO₃), 0% to 100% gradient in 30 min; detector: UV 254 nm, to afford 120-2 (456 mg, 56.42%) as a yellow solid.

Step 3: Synthesis of 120-3. A solution of 120-2 (478 mg, 1.530 mmol, 1 eq.), NH₂OH·HCl (159.50 mg, 2.295 mmol, 1.50 eq.) and NaOAc (188.29 mg, 2.295 mmol, 1.5 eq.) in MeOH (15 mL) was stirred for 2 days at 50° C. After the reaction was completed, the solvent was removed under vacuum to give the crude product. The residue was purified by reversed-phase flash chromatography with the following conditions: column: C18 silica gel; mobile phase: ACN in water (10 mmol/L NH₄CO₃), 0 to 100% gradient in 25 min; detector: UV 254 nm, to give 120-3 (198 mg, 87.33%) as light-yellow solid.

Step 4: Synthesis of 120-4. A solution of 120-3 (160 mg, 1.080 mmol, 1 eq.) and 1-(triphenylmethyl)pyrazole-4-sulfonylchloride (662.32 mg, 1.62 mmol, 1.5 eq.) in pyridine (10 mL) was stirred overnight at 80° C. After the reaction was completed, the solvent was removed under vacuum to give a residue. The crude was purified by reversed-phase flash chromatography with the following conditions: column: C18 silica gel; mobile phase: ACN in water (10 mmol/L NH₄CO₃), 0% to 100% gradient in 30 min; detector: UV 254 nm, to give 120-4 (180 mg, 32.02%) as a yellow solid.

Step 5: Synthesis of 120-5. A solution of 120-4 (200 mg, 0.384 mmol, 1 eq.) in 4 N HCl/MeOH (20 mL) was stirred for 2 days. After the reaction was completed, the solvent was removed to give the crude product. The residue was purified by reversed-phase flash chromatography with the following conditions: column: C18 silica gel; mobile phase: ACN in water (10 mmol/L NH₄CO₃), 0% to 100% gradient in 30 min; detector: UV 254 nm, to afford 120-5 (180 mg) as a yellow solid.

Step 6: Synthesis of I-120. A solution of 120-5 (170 mg, 0.61 mmol, 1 eq.), 2-chloro-4-(1,1-difluoroethyl)pyridine (130.17 mg, 0.73 mmol, 1.2 eq.) and Cs₂CO₃ (398.07 mg, 1.22 mmol, 2 eq.) in DMF (7 mL) was stirred overnight at 80° C. After the reaction was completed, the mixture was added to water (40 mL), extracted with EtOAc (4×50 mL), and then the organic phase was combined, washed with the brine (50 mL), dried over anhydrous Na₂SO₄, and concentrated to get crude product. The residue was purified by reversed-phase flash chromatography with the following conditions: column: C18 silica gel; mobile phase: ACN in water (10 mmol/L NH₄CO₃), 0% to 100% gradient in 30 min; detector: UV 254 nm, to the crude. The residue was purified by prep-HPLC with the following conditions: column: Xselect CSH C₁₈ OBD column 30*150 mm 5 μm; mobile phase A: water (0.1% FA), mobile phase B: ACN; flow rate: 60 mL/min; gradient: 22% B to 33% B in 7 min; wavelength: 254 nm/220 nm; Rt(min): 6.85) to afford I-120 (20.1 mg, 7.81%) as an off-white solid. (ES, m/z): [M+H]⁺ 420; ¹H NMR (400 MHz, DMSO-d₆) δ 2.04 (t, J=18.8 Hz, 3H), 4.38 (s, 3H), 7.63 (d, J=4.4 Hz, 1H), 7.76 (s, 1H), 8.02 (s, 1H), 8.07 (s, 1H), 8.14 (s, 1H), 8.33 (s, 1H), 8.66 (d, J=5.2 Hz, 1H), 8.76 (s, 1H), 8.82 (s, 1H), 12.26 (s, 1H).

### Example 215—Synthesis of 1-(5-Chloro-4-(Trifluoromethyl)Pyridin-2-yl)-N-(1-Methyl-1H-Indazol-7-yl)-1H-Pyrazole-4-Sulfonamide (I-121)

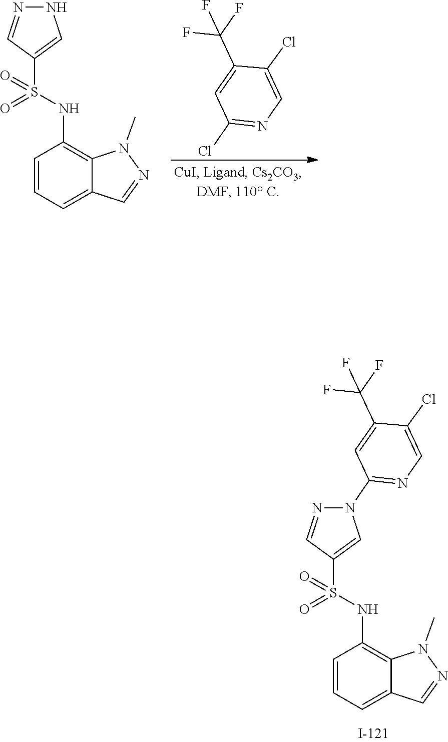 [SMILES: Cn1ncc2cccc(NS(=O)(=O)c3cn[nH]c3)c21 ; FC(F)(F)c1cc(Cl)ncc1Cl ; Cn1ncc2cccc(NS(=O)(=O)c3cnn(-c4cc(C(F)(F)F)c(Cl)cn4)c3)c21]

A solution of N-(1-methylindazol-7-yl)-1H-pyrazole-4-sulfonamide (120 mg, 0.433 mmol, 1 eq.), 2,5-dichloro-4-(trifluoromethyl)pyridine (140.20 mg, 0.649 mmol, 1.5 eq.), CuI (82.42 mg, 0.433 mmol, 1 eq.), N1,N2-dimethylcyclohexane-1,2-diamine (61.56 mg, 0.433 mmol, 1 eq.) and Cs₂CO₃ (422.99 mg, 1.299 mmol, 3 eq.) in DMF (2 mL) was stirred for 16 h at 110° C. under a nitrogen atmosphere. The residue was dissolved in water (10 mL). The resulting mixture was extracted with EtOAc (3×15 mL). The combined organic layers were washed with brine (20 mL), and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₃·H₂O), 10% to 50% gradient in 10 min; detector: UV 254 nm. This resulted in 1-[5-chloro-4-(trifluoromethyl)pyridin-2-yl]-N-(1-methylindazol-7-yl)pyrazole-4-sulfonamide (59.7 mg, 29.38%) as a white solid. (ES, m/z): [M+H]⁺ 457; ¹H NMR (400 MHz, DMSO-d₆) δ 4.28 (s, 3H), 6.72 (d, J=6.8 Hz, 1H), 6.97 (t, J=7.6 Hz, 1H), 7.70 (d, J=8.0 Hz, 1H), 8.09 (s, 1H), 8.11 (s, 1H), 8.25 (s, 1H), 8.85 (s, 1H), 8.93 (s, 1H), 10.11 (s, 1H).

### Example 216—Synthesis of N-(1-Methyl-1H-Indazol-7-yl)-1-(4-(Trifluoromethyl)Pyridin-2-yl)-1H-Pyrazole-4-Sulfonamide

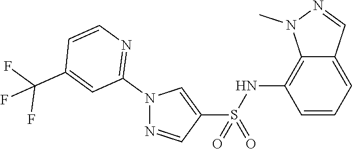 [SMILES: Cn1ncc2cccc(NS(=O)(=O)c3cnn(-c4cc(C(F)(F)F)ccn4)c3)c21]

A solution of 1-(4-(trifluoromethyl)pyridin-2-yl)-1H-pyrazole-4-sulfonyl chloride (1.00 g, 3.209 mmol, 1 eq.) and 1-methyl-1H-indazol-7-amine (0.52 g, 3.530 mmol, 1.1 eq.) in pyridine (10 mL) was stirred for 2 h at room temperature under a nitrogen atmosphere. The resulting mixture was diluted with water (100 mL). The resulting mixture was extracted with EtOAc (3×100 mL). The combined organic layers were washed with brine (100 mL), and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 70% to 80% gradient in 10 min; detector: UV 254 nm. This resulted in N-(1-methyl-1H-indazol-7-yl)-1-(4-(trifluoromethyl)pyridin-2-yl)-1H-Pyrazole-4-sulfonamide (700.00 mg, 49.07%) as a light yellow solid.

### Example 217—Synthesis of N-(2-(Dimethylamino)Ethyl)-N-(1-Methyl-1H-Indazol-7-yl)-1-(4-(Trifluoromethyl)Pyridin-2-yl)-1H-Pyrazole-4-Sulfonamide (I-188)

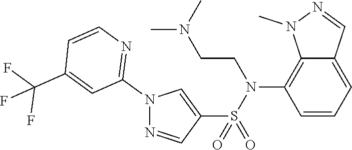 [SMILES: CN(C)CCN(c1cccc2cnn(C)c12)S(=O)(=O)c1cnn(-c2cc(C(F)(F)F)ccn2)c1]

A mixture of N-(1-methyl-1H-indazol-7-yl)-1-(4-(trifluoromethyl)pyridin-2-yl)-1H-pyrazole-4-sulfonamide (150.00 mg, 0.355 mmol, 1 eq.), 2-bromo-N,N-dimethylethan-1-amine (64.79 mg, 0.426 mmol, 1.2 eq.) and Cs₂CO₃ (347.12 mg, 1.065 mmol, 3 eq.) in DMF (4 mL) was stirred for 2 h at room temperature. The resulting mixture was extracted with EtOAc (3×15 mL). The combined organic layers were washed with brine (10 mL) and dried over anhydrous Na₂SO₄. After filtration, the filtrate was concentrated under reduced pressure. The crude product was purified by prep-HPLC with the following conditions: flow rate: 60 mL/min; gradient: 42% B to 70% B in 7 min; wavelength: 254 nm/220 nm; Rt(min): 7.4, to afford N-(2-(dimethylamino)ethyl)-N-(1-methyl-1H-indazol-7-yl)-1-(4-(trifluoromethyl)pyridin-2-yl)-1H-pyrazole-4-sulfonamide (26.90 mg, 14.60%) as an off-white solid. (ES, m/z): [M+H]⁺=494; ¹H NMR (400 MHz, DMSO-d₆) δ 2.04 (s, 6H), 2.10-2.33 (m, 2H), 3.42-3.48 (m, 1H), 4.10-4.17 (m, 1H), 4.37 (s, 3H), 6.92 (d, J=7.2 Hz, 1H), 7.04 (t, J=7.6 Hz, 1H), 7.78 (d, J=8.0 Hz, 1H), 7.91 (d, J=4.8 Hz, 1H), 8.10 (s, 1H), 8.11 (s, 1H), 8.24 (s, 1H), 8.85 (d, J=5.2 Hz, 1H), 9.08 (s, 1H).

### Example 218—Synthesis of N-(1-Methyl-1H-Indazol-7-yl)-1-(4-(Trifluoromethyl)Pyridin-2-yl)-1H-pyrazole-4-Sulfonamide

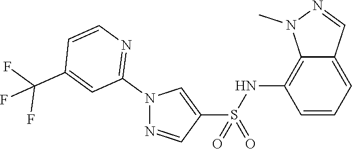 [SMILES: Cn1ncc2cccc(NS(=O)(=O)c3cnn(-c4cc(C(F)(F)F)ccn4)c3)c21]

A solution of 1-(4-(trifluoromethyl)pyridin-2-yl)-1H-pyrazole-4-sulfonyl chloride (1 g, 3.209 mmol, 1.00 eq.) and 1-methyl-1H-indazol-7-amine (0.52 g, 3.530 mmol, 1.10 eq.) in pyridine (10 mL) was stirred for 2 h at room temperature under a nitrogen atmosphere. The resulting mixture was diluted with water (100 mL). The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 70% to 80% gradient in 10 min; detector: UV 254 nm. This resulted in N-(1-methyl-1H-indazol-7-yl)-1-(4-(trifluoromethyl)pyridin-2-yl)-1H-pyrazole-4-sulfonamide (700 mg, 49.07%) as a light yellow solid.

### Example 219—Synthesis of N-(3-(Dimethylamino)Propyl)-N-(1-Methyl-1H-Indazol-7-yl)-1-(4-(Trifluoromethyl)Pyridine-2-yl)-1H-Pyrazole-4-Sulfonamide (I-189)

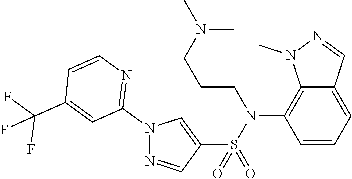 [SMILES: CN(C)CCCN(c1cccc2cnn(C)c12)S(=O)(=O)c1cnn(-c2cc(C(F)(F)F)ccn2)c1]

A solution of N-(1-methyl-1H-indazol-7-yl)-1-(4-(trifluoromethyl)pyridin-2-yl)-1H-pyrazole-4-sulfonamide (130 mg, 0.308 mmol, 1.00 eq.), (3-bromopropyl)dimethylamine (61.33 mg, 0.370 mmol, 1.20 eq.) and Cs₂CO₃ (300.83 mg, 0.924 mmol, 3.00 eq.) in DMF (2 mL) was stirred for 2 h at 90° C. under a nitrogen atmosphere. The resulting mixture was diluted with water (10 mL). The resulting mixture was extracted with EtOAc (3×10 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 40% B to 65% B in 7 min; wavelength: 220 nm; Rt(min): 7.8 to afford N-(3-(dimethylamino)propyl)-N-(1-methyl-1H-indazol-7-yl)-1-(4-(trifluoromethyl)pyridine-2-yl)-1H-Pyrazole-4-sulfonamide (22.9 mg, 14.63%) as an off-white solid. (ES, m/z): [M+H]⁺ 508; ¹H NMR: (400 MHz, DMSO-d₆) δ 1.30-1.50 (m, 1H), 1.60-1.80 (m, 1H), 1.98 (s, 6H), 2.17 (t, J=6.8 Hz, 2H), 3.35-3.60 (m, 1H), 3.90-4.15 (m, 1H), 4.32 (s, 3H), 6.95 (d, J=7.6 Hz, 1H), 7.07 (t, J=7.6 Hz, 1H), 7.80 (d, J=8.0 Hz, 1H), 7.92 (d, J=5.2 Hz, 1H), 8.10 (s, 1H), 8.15 (s, 1H), 8.24 (s, 1H), 8.86 (d, J=5.2 Hz, 1H), 9.03 (s, 1H).

### Example 220—Synthesis of 1-(4-(3-Hydroxyoxetan-3-yl)Pyridin-2-yl)-N-(6-Methoxy-1-Methyl-1H-Indazol-7-yl)-1H-Pyrazole-4-Sulfonamide (I-190)

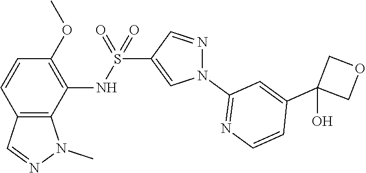 [SMILES: COc1ccc2cnn(C)c2c1NS(=O)(=O)c1cnn(-c2cc(C3(O)COC3)ccn2)c1]

To a stirred solution of N-(6-methoxy-1-methylindazol-7-yl)-1H-Pyrazole-4-sulfonamide (400 mg, 1.302 mmol, 1 eq.) and 3-(2-fluoropyridin-4-yl)oxetan-3-ol (264.19 mg, 1.562 mmol, 1.2 eq.) in NMP (5 mL) was added Cs₂CO₃ (1272.19 mg, 3.906 mmol, 3 eq.) in portions at room temperature under a nitrogen atmosphere. The resulting mixture was stirred for 16 h at 90° C. under a nitrogen atmosphere. Desired product could be detected by LCMS. The reaction was quenched with sat. NH4Cl (aq.) at room temperature. The resulting mixture was extracted with CH₂Cl₂ (3×30 mL). The combined organic layers were washed with brine (5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in 1-(4-(3-hydroxyoxetan-3-yl)pyridin-2-yl)-N-(6-methoxy-1-methyl-1H-indazol-7-yl)-1H-Pyrazole-4-sulfonamide (66.1 mg, 10.95%) as a pink solid. (ES, m/z): [M+H]⁺=457; ¹H NMR (400 MHz, DMSO-d₆) δ 3.33 (s, 3H), 4.24 (s, 3H), 4.69 (d, J=6.4 Hz, 2H), 4.85 (d, J=6.4 Hz, 2H), 6.86-6.89 (m, 2H), 7.67-7.72 (m, 2H), 7.98-8.00 (m, 2H), 8.19 (s, 1H), 8.55 (d, J=5.2 Hz, 1H), 8.73 (s, 1H), 9.80 (s, 1H).

### Example 221—Synthesis of 1-(4-Cyclopropylpyridin-2-yl)-N-(6-Methoxy-1-Methyl-1H-Indazol-7-yl)-N-Methyl-1H-Pyrazole-4-Sulfonamide (I-191)

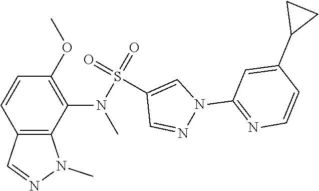 [SMILES: COc1ccc2cnn(C)c2c1N(C)S(=O)(=O)c1cnn(-c2cc(C3CC3)ccn2)c1]

To a stirred solution of 1-(4-cyclopropylpyridin-2-yl)-N-(6-methoxy-1H-indazol-7-yl)-N-methylpyrazole-4-sulfonamide (100 mg, 0.236 mmol, 1 eq.) and Cs₂CO₃ (230.27 mg, 0.708 mmol, 3 eq.) in DMF (2 mL) was added Mel (33.44 mg, 0.236 mmol, 1 eq.) dropwise at room temperature under a nitrogen atmosphere. The resulting mixture was stirred for 1 h at room temperature under a nitrogen atmosphere. The reaction was quenched with water (10 mL). The resulting mixture was extracted with CH₂Cl₂ (3×10 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. This resulted in 1-(4-cyclopropylpyridin-2-yl)-N-(6-methoxy-1-methyl-1H-indazol-7-yl)-N-methyl-1H-pyrazole-4-sulfonamide (79.5 mg, 76.42%) as a white solid. (ES, m/z): [M+H]⁺ 439; ¹H NMR: (400 MHz, DMSO-d₆) δ 0.93-0.95 (m, 2H), 1.14-1.19 (m, 2H), 2.11-2.18 (m, 1H), 3.29 (s, 3H), 3.41 (s, 3H), 4.21 (s, 3H), 6.94 (d, J=8.8 Hz, 1H), 7.16 (dd, J=5.2, 1.2 Hz, 1H), 7.73-7.75 (m, 2H), 8.00 (s, 1H), 8.13 (s, 1H), 8.34 (d, J=5.2 Hz, 1H), 8.86 (s, 1H).

### Example 222—Synthesis of 1-(4-Bromopyridin-2-yl)-N-(6-Methoxy-1-Methyl-1H-Indazol-7-yl)-1H-Pyrazole-4-Sulfonamide

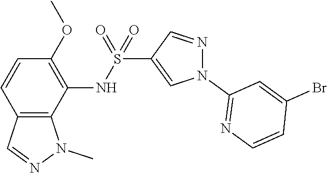 [SMILES: COc1ccc2cnn(C)c2c1NS(=O)(=O)c1cnn(-c2cc(Br)ccn2)c1]

A mixture of 4-bromo-2-fluoropyridine (458.11 mg, 2.604 mmol, 2 eq.), Cs₂CO₃ (1272.19 mg, 3.906 mmol, 3 eq.) and N-(6-methoxy-1-methylindazol-7-yl)-1H-pyrazole-4-sulfonamide (400 mg, 1.302 mmol, 1 eq.) in MeCN (40 mL) was stirred for 12 h at 80° C. under a nitrogen atmosphere. The reaction was quenched with water at room temperature. The aqueous layer was extracted with EtOAc (3×50 mL). The combined organic layers were washed with brine (5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 70% to 100% gradient in 10 min; detector: UV 254 nm, to afford 1-(4-bromopyridin-2-yl)-N-(6-methoxy-1-Methyl-1H-Indazol-7-yl)-1H-pyrazole-4-sulfonamide (300 mg, 49.75%) as a yellow solid.

### Example 223—Synthesis of 1-(4-(3-Hydroxyprop-1-EN-2-yl)Pyridin-2-yl)-N-(6-Methoxy-1-Methyl-1H-Indazol-7-yl)-1H-Pyrazole-4-Sulfonamide (I-68)

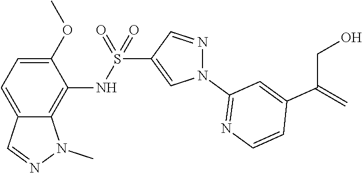 [SMILES: C=C(CO)c1ccnc(-n2cc(S(=O)(=O)Nc3c(OC)ccc4cnn(C)c34)cn2)c1]

To a stirred mixture of 1-(4-bromopyridin-2-yl)-N-(6-methoxy-1-methyl-1H-indazol-7-yl)-1H-pyrazole-4-sulfonamide (300 mg, 0.648 mmol, 1 eq.) and 4,4,5,5-tetramethyl-2-(oxetan-3-yl)-1,3,2-dioxaborolane (237.93 mg, 1.296 mmol, 2 eq.) in H₂O (2.5 mL) and dioxane (14 mL) were added Cs₂CO₃ (421.95 mg, 1.296 mmol, 2 eq.) and Pd(dppf)Cl₂ (94.76 mg, 0.130 mmol, 0.2 eq.) in portions at room temperature under a nitrogen atmosphere. The resulting mixture was stirred for two days at 80° C. under a nitrogen atmosphere. Desired product could be detected by LCMS. The residue was purified with the following conditions: column: CHIRALPAK IH, 2*25 cm, 5 μm; mobile phase A: hex (0.5% 2M NH₃-MeOH), mobile phase B: EtOH:DCM=1:1-HPLC; flow rate: 20 mL/min; gradient: 30% B to 30% B in 12 min; wavelength: 220/254 nm; Rt(min): 8.27; Rt2(min): 11.15; sample solvent: EtOH:DCM=1:1; injection volume: 0.6 mL. This resulted in 1-(4-(3-hydroxyprop-1-en-2-yl)pyridin-2-yl)-N-(6-methoxy-1-methyl-1H-indazol-7-yl)-1H-pyrazole-4-sulfonamide (6.9 mg, 2.40%) as a white solid (ES, m/z): [M+H]⁺ 441; ¹H NMR: (400 MHz, DMSO-d₆) δ 3.34 (s, 3H), 4.25 (s, 3H), 4.41 (d, J=5.2 Hz, 2H), 5.24 (t, J=5.2 Hz, 1H), 5.60 (s, 1H), 5.87 (s, 1H), 6.88 (d, J=8.8 Hz, 1H), 7.57 (d, J=5.2 Hz, 1H), 7.68 (d, J=8.8 Hz, 1H), 7.98-8.02 (m, 3H), 8.48 (d, J=5.2 Hz, 1H), 8.72 (s, 1H), 9.78 (s, 1H).

### Example 224—Synthesis of N-(4-Bromobutyl)-N-(1-Methyl-1H-Indazol-7-yl)-1-(4-(Trifluoromethyl)Pyridin-2-yl)-1H-Pyrazole-4-Sulfonamide

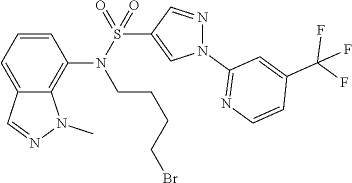 [SMILES: Cn1ncc2cccc(N(CCCCBr)S(=O)(=O)c3cnn(-c4cc(C(F)(F)F)ccn4)c3)c21]

A solution of N-(1-methyl-1H-indazol-7-yl)-1-(4-(trifluoromethyl)pyridin-2-yl)-1H-pyrazole-4-sulfonamide (150 mg, 0.355 mmol, 1.00 eq.), 1,4-dibromobutane (92.0 mg, 0.426 mmol, 1.20 eq.) and Cs₂CO₃ (231.4 mg, 0.710 mmol, 2.00 eq.) in DMF (5 mL) was stirred for 2 h at 90° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (6:1) to afford N-(4-bromobutyl)-N-(1-methyl-1H-indazol-7-yl)-1-(4-(trifluoromethyl)pyridin-2-yl)-1H-pyrazole-4-sulfonamide (78 mg, crude) as a light yellow oil.

### Example 225—Synthesis of N-(4-(Dimethylamino)butyl)-N-(1-Methyl-1H-Indazol-7-yl)-1-(4-(Trifluoromethyl)Pyridin-2-yl)-1H-Pyrazole-4-Sulfonamide (I-192)

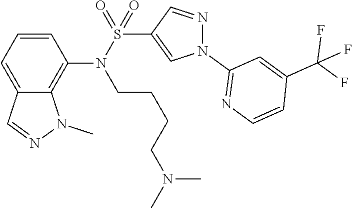 [SMILES: CN(C)CCCCN(c1cccc2cnn(C)c12)S(=O)(=O)c1cnn(-c2cc(C(F)(F)F)ccn2)c1]

A solution of N-(4-bromobutyl)-N-(1-methyl-1H-indazol-7-yl)-1-(4-(trifluoromethyl)pyridin-2-yl)-1H-pyrazole-4-sulfonamide (78.0 mg, 0.140 mmol, 1.00 eq.), Cs₂CO₃ (91.1 mg, 0.280 mmol, 2.00 eq.) and dimethylamine (7.5 mg, 0.168 mmol, 1.20 eq.) in DMF (5 mL) was stirred for 2 h at 80° C. under a nitrogen atmosphere. The mixture was allowed to cool down to room temperature. The resulting mixture was diluted with water (10 mL). The resulting mixture was extracted with EtOAc (3×10 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 36% B to 64% B in7 min; wavelength: 254 nm/220 nm; Rt(min):7.05 to afford N-(4-(dimethylamino)butyl)-N-(1-methyl-1H-indazol-7-yl)-1-(4-(trifluoromethyl)pyridin-2-yl)-1H-Pyrazole-4-sulfonamide as an off-white solid. (ES, m/z): [M+H]⁺ 522; ¹H NMR: (400 MHz, DMSO-d₆) δ 1.22-1.51 (m, 4H), 1.99 (s, 6H), 2.08 (t, J=6.8 Hz, 2H), 3.37-.346 (m, 1H), 3.90-4.10 (m, 1H), 4.31 (s, 3H), 6.92 (d, J=7.6 Hz, 1H), 7.06 (t, J=7.6 Hz, 1H), 7.79 (d, J=8.0 Hz, 1H), 7.92 (d, J=5.2, 1H), 8.10 (s, 1H), 8.14 (s, 1H), 8.24 (s, 1H), 8.86 (d, J=5.2 Hz, 1H), 9.06 (s, 1H).

### Example 226—Synthesis of 1-(4-Cyclopropylpyridin-2-yl)-N-(1-Methyl-1H-Indazol-7-yl)-1H-Pyrazole-4-Sulfonamide

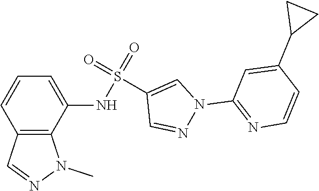 [SMILES: Cn1ncc2cccc(NS(=O)(=O)c3cnn(-c4cc(C5CC5)ccn4)c3)c21]

To a stirred solution of N-(1-methylindazol-7-yl)-1H-pyrazole-4-sulfonamide (200 mg, 0.721 mmol, 1 eq.) and 4-cyclopropyl-2-fluoropyridine (98.92 mg, 0.721 mmol, 1 eq.) in DMF (2 mL) was added Cs₂CO₃ (704.98 mg, 2.163 mmol, 3 eq.) at 110° C. under a nitrogen atmosphere. The resulting mixture was stirred overnight at 110° C. under a nitrogen atmosphere. The reaction was quenched with water (10 mL). The resulting mixture was extracted with EtOAc (3×10 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 1-(4-cyclopropylpyridin-2-yl)-N-(1-methyl-1H-indazol-7-yl)-1H-pyrazole-4-sulfonamide (100 mg, 31.64%) as a yellow solid.

### Example 227—Synthesis of 1-(4-Cyclopropylpyridin-2-yl)-N-Methyl-N-(1-Methyl-1H-Indazol-7-yl)-1H-Pyrazole-4-Sulfonamide (I-193)

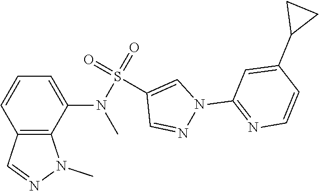 [SMILES: CN(c1cccc2cnn(C)c12)S(=O)(=O)c1cnn(-c2cc(C3CC3)ccn2)c1]

To a stirred solution of 1-(4-cyclopropylpyridin-2-yl)-N-(1-methyl-1H-indazol-7-yl)-1H-pyrazole-4-sulfonamide (100 mg, 0.254 mmol, 1 eq.) and Cs₂CO₃ (57.90 mg, 0.762 mmol, 3 eq.) in DMF (1 mL) was added methyl iodide (0.02 mL, 0.254 mmol, 1 eq.) dropwise at room temperature under a nitrogen atmosphere. The resulting mixture was stirred for 1 h at room temperature under a nitrogen atmosphere. The reaction was quenched with water (10 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 1-(4-cyclopropylpyridin-2-yl)-N-methyl-N-(1-methyl-1H-indazol-7-yl)-1H-pyrazole-4-sulfonamide (6.6 mg, 6.32%) as a white solid. (ES, m z): [M+H]⁺ 409; ¹H NMR (400 MHz, DMSO-d₆) δ 0.92-0.95 (m, 2H), 1.16-1.18 (m, 2H), 2.15-2.16 (m, 1H), 4.29 (s, 3H), 6.88 (d, J=7.6, 1H), 7.04 (t, J=8.0, 1H), 7.20 (dd, J=5.2, 1.6 Hz, 1H), 7.73 (s, 1H), 7.78 (dd, J=8.0, 0.8 Hz, 1H), 8.04 (s, 1H), 8.12 (s, 1H), 8.35 (d, J=5.2 Hz, 1H), 8.92 (d, J=0.8 Hz, 1H).

### Example 228—Synthesis of 2-Fluoro-4-(3-Methoxyoxetan-3-yl)Pyridine

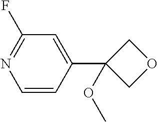

A solution of 3-(2-fluoropyridin-4-yl)oxetan-3-ol (400 mg, 2.365 mmol, 1 eq.) in DMF (8 mL) was treated with NaH (85.12 mg, 3.548 mmol, 1.5 eq.) for 30 min at 0° C. under a nitrogen atmosphere followed by the addition of methyl iodide (503.46 mg, 3.548 mmol, 1.5 eq.) dropwise at 0° C. The mixture was stirred for 3 h at room temperature under a nitrogen atmosphere. The reaction was quenched by the addition of water (10 mL) at 0° C. The resulting mixture was extracted with EtOAc (3×30 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm, to afford 2-fluoro-4-(3-methoxyoxetan-3-yl)pyridine (100 mg, 23.09%) as a yellow oil.

### Example 229—Synthesis of N-(6-Methoxy-1-Methyl-1H-Indazol-7-yl)-1-(4-(3-Methoxyoxetan-3-yl)Pyridin-2-yl)-1H-Pyrazole-4-Sulfonamide (I-194)

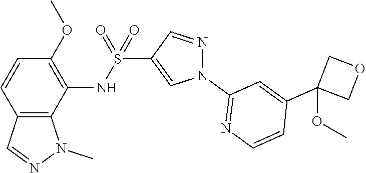 [SMILES: COc1ccc2cnn(C)c2c1NS(=O)(=O)c1cnn(-c2cc(C3(OC)COC3)ccn2)c1]

A mixture of 2-fluoro-4-(3-methoxyoxetan-3-yl)pyridine (71.53 mg, 0.390 mmol, 3 eq.), Cs₂CO₃ (169.63 mg, 0.520 mmol, 4 eq.) and N-(6-methoxy-1-methylindazol-7-yl)-1H-pyrazole-4-sulfonamide (40 mg, 0.130 mmol, 1.00 eq.) in MeCN (5 mL) was stirred for 3 days at 80° C. under a nitrogen atmosphere. The reaction was quenched by the addition of water (10 mL) at 0° C. The resulting mixture was extracted with EtOAc (3×30 mL). The combined organic layers were washed with brine (10 mL), and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm, to afford N-(6-methoxy-1-methyl-1H-indazol-7-yl)-1-(4-(3-methoxyoxetan-3-yl)pyridin-2-yl)-1H-Pyrazole-4-sulfonamide (31.5 mg, 51.44%) as a white solid. (ES, m/z): [M+H]⁺ 471; ¹H NMR: (400 MHz, DMSO-d₆) δ 3.20 (s, 3H), 3.35 (s, 3H), 4.25 (s, 3H), 4.74 (d, J=7.6 Hz, 2H) 4.89 (d, J=7.6 Hz, 2H), 6.88 (d, J=8.8 Hz, 1H), 7.62 (dd, J=5.2, 1.6 Hz, 1H), 7.68 (d, J=8.8 Hz, 1H), 7.98-8.03 (m, 3H), 8.61 (d, J=5.2 Hz, 1H), 8.74 (s, 1H), 9.80 (s, 1H).

### Example 230—Synthesis of (E)-1-(4-(3-Hydroxyprop-1-EN-1-yl)Pyridin-2-yl)-N-(6-Methoxy-1-Methyl-1H-Indazol-7-yl)-1H-Pyrazole-4-Sulfonamide (I-195)

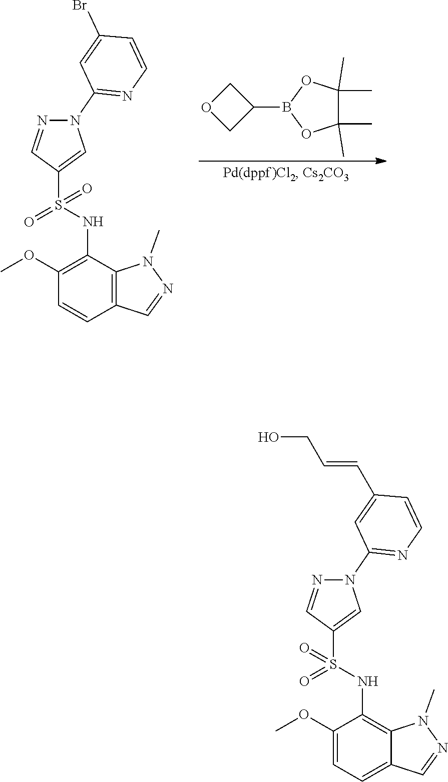 [SMILES: COc1ccc2cnn(C)c2c1NS(=O)(=O)c1cnn(-c2cc(Br)ccn2)c1 ; CC1(C)OB(C2COC2)OC1(C)C ; COc1ccc2cnn(C)c2c1NS(=O)(=O)c1cnn(-c2cc(/C=C/CO)ccn2)c1]

To a stirred mixture of 1-(4-bromopyridin-2-yl)-N-(6-methoxy-1-methyl-1H-indazol-7-yl)-1H-pyrazole-4-sulfonamide (300 mg, 0.648 mmol, 1 eq.) and 4,4,5,5-tetramethyl-2-(oxetan-3-yl)-1,3,2-dioxaborolane (238.34 mg, 1.296 mmol, 2 eq.) in H₂O (2.5 mL) and dioxane (14 mL) were added Cs₂CO₃ (421.95 mg, 1.296 mmol, 2 eq.) and Pd(dppf)Cl₂ (94.76 mg, 0.130 mmol, 0.2 eq.) in portions at room temperature under a nitrogen atmosphere. The resulting mixture was stirred for two days at 80° C. under a nitrogen atmosphere. The residue was purified with the following conditions: column: CHIRALPAK IH, 2*25 cm, 5 μm; mobile phase A: hex (0.5% 2 M NH₃-MeOH), mobile phase B: EtOH:DCM=1:1; flow rate: 20 mL/min; gradient: 30% B to 30% B in 12 min; wavelength: 220/254 nm; Rt(min): 8.27. This resulted in (E)-1-(4-(3-hydroxyprop-1-en-1-yl)pyridin-2-yl)-N-(6-methoxy-1-methyl-1H-indazol-7-yl)-1H-pyrazole-4-sulfonamide (2.7 mg, 0.94%, white solid). (ES, m/z): [M+H]⁺=441; ¹H NMR: (400 MHz, DMSO-d₆) δ 3.35 (s, 3H), 4.21-4.24 (m, 5H), 5.09 (t, J=5.2 Hz, 1H), 6.71 (d, J=15.6 Hz, 1H), 6.83-6.90 (m, 2H), 7.52 (d, J=5.2 Hz, 1H), 7.67 (d, J=8.8 Hz, 1H), 7.94 (s, 1H), 7.98 (s, 2H), 8.43 (d, J=5.2 Hz, 1H), 8.69 (s, 1H), 9.77 (s, 1H).

### Example 231—Synthesis of 3-Bromo-1-Methyl-5-(Trifluoromethyl)-1H-Pyrazole

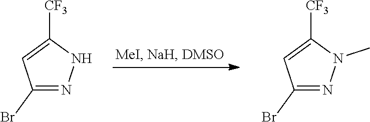

To a solution of 3-bromo-5-(trifluoromethyl)-1H-pyrazole (350 mg, 1.628 mmol, 1 eq.) in DMSO (2 mL) was added NaH (78 mg, 3.250 mmol, 2.00 eq.) at 0° C. under a nitrogen atmosphere. The resulting solution was stirred for 15 min and followed by the addition of Mel (450 mg, 3.170 mmol, 1.95 eq.) dropwise. The resulting solution was stirred overnight at room temperature under a nitrogen atmosphere. The reaction solution containing 3-bromo-1-methyl-5-(trifluoromethyl)-1H-pyrazole was used in next step directly.

### Example 232—Synthesis of 1′-Methyl-N-(1-Methylindazol-7-yl)-5′-(Trifluoromethyl)-[1,3′-Biyrazole]-4-Sulfonamide (I-196)

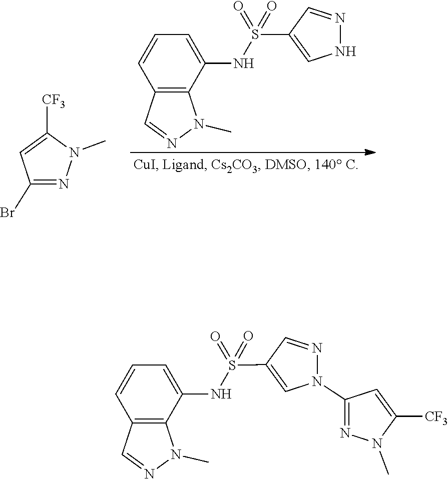

N-(1-methylindazol-7-yl)-1H-pyrazole-4-sulfonamide (80 mg, 0.288 mmol, 0.94 eq.), CuI (116 mg, 0.609 mmol, 1.99 eq.), 3-bromo-1-methyl-5-(trifluoromethyl)-1H-pyrazole (300 mg, 0.434 mmol, 1.42 eq.) and Cs₂CO₃ (200 mg, 0.614 mmol, 2.01 eq.) was added to the reaction. The resulting mixture was stirred overnight at 140° C. under a nitrogen atmosphere. The resulting mixture was diluted with water (20 mL). The resulting mixture was extracted with EtOAc (3×15 mL). The combined organic layers were washed with water (3×10 mL) and dried over anhydrous Na₂SO₄. After filtration, the filtrate was concentrated under reduced pressure. The crude product (140 mg) was purified by prep-HPLC with the following conditions: column: XBridge Prep OBD C18 column, 19*250 mm, 5 μm; mobile phase A: water (10 mmol/L NH₄CO₃+0.05% NH₃H₂O), mobile phase B: ACN (1% 2 mM NH₃-MEOH); flow rate: 25 mL/min; gradient: 42% B to 67% B in 10 min; wavelength: 254 nm/220 nm) 1′-methyl-N-(1-methylindazol-7-yl)-5′-(trifluoromethyl)-[1,3′-bipyrazole]-4-sulfonamide (18.9 mg, 14.36%) was obtained as an off-white solid. (ES, m/z): [M+H]⁺ 426; ¹H NMR: (400 MHz, DMSO-d₆) δ 4.04 (s, 3H), 4.28 (s, 3H), 6.71 (d, J=7.2 Hz, 1H), 6.98 (t, J=7.6 Hz, 1H), 7.30 (s, 1H), 7.67 (d, J=8.0 Hz, 1H), 7.97 (s, 1H), 8.07 (s, 1H), 8.59 (s, 1H), 10.01 (s, 1H).

### Example 233—Synthesis of 1′-Methyl-N-(1-Methylindazol-7-yl)-5′-(Trifluoromethyl)-[1,3′-Bipyrazole]-4-Sulfonamide (I-197)

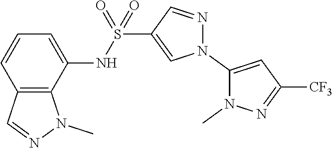 [SMILES: Cn1nc(C(F)(F)F)cc1-n1cc(S(=O)(=O)Nc2cccc3cnn(C)c23)cn1]

N-(1-methylindazol-7-yl)-1H-pyrazole-4-sulfonamide (80 mg, 0.288 mmol, 0.94 eq.), CuI (116 mg, 0.609 mmol, 1.99 eq.), 3-bromo-1-methyl-5-(trifluoromethyl)-1H-pyrazole (300 mg, 0.434 mmol, 1.42 eq.) and Cs₂CO₃ (200 mg, 0.614 mmol, 2.01 eq.) was added to the reaction. The resulting mixture was stirred overnight at 140° C. under a nitrogen atmosphere. The resulting mixture was diluted with water (20 mL). The resulting mixture was extracted with EtOAc (3×15 mL). The combined organic layers were washed with water (3×10 mL) and dried over anhydrous Na₂SO₄. After filtration, the filtrate was concentrated under reduced pressure. The crude product (140 mg) was purified by prep-HPLC with the following conditions: column: XBridge Prep OBD C18 column, 19*250 mm, 5 μm; mobile phase A: water (10 mmol/L NH₄CO₃+0.05% NH₃H₂O), mobile phase B: ACN (1% 2 mM NH₃-MEOH); flow rate: 25 mL/min; gradient: 42% B to 67% B in 10 min; wavelength: 254 nm/220 nm). 2′-methyl-N-(1-methylindazol-7-yl)-5′-(trifluoromethyl)-[1,3′-bipyrazole]-4-sulfonamide (10.7 mg, 8.07%) was obtained as an off-white solid. (ES, m/z): [M+H]⁺ 426; ¹H NMR: (400 MHz, DMSO-d₆) δ 3.86 (s, 3H), 4.28 (s, 3H), 6.75 (d, J=7.2 Hz, 1H), 7.01 (t, J=7.6 Hz, 1H), 7.21 (s, 1H), 7.68 (d, J=7.6 Hz, 1H), 8.07 (s, 1H), 8.12 (s, 1H), 8.70 (s, 1H), 10.09 (s, 1H).

### Example 234—Synthesis of N-(6-Methoxy-1-Methyl-1H-Pyrazolo[4,3-C]Pyridin-7-yl)-1-Trityl-1H-Pyrazole-4-Sulfonamide

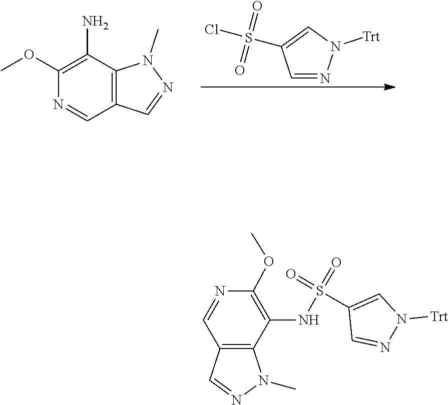

A solution of 6-methoxy-1-methylpyrazolo[4,3-c]pyridin-7-amine (200 mg, 1.122 mmol, 1 eq.) and 1-(triphenylmethyl)pyrazole-4-sulfonyl chloride (458.94 mg, 1.122 mmol, 1 eq.) in pyridine (2 mL) was stirred for 2 h at room temperature under a nitrogen atmosphere. The resulting mixture was concentrated under vacuum. The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in N-(6-methoxy-1-methyl-1H-pyrazolo[4,3-c]pyridin-7-yl)-1-trityl-1H-Pyrazole-4-sulfonamide (142 mg, 20.68%) as a yellow solid.

### Example 235—Synthesis of N-(6-Methoxy-1-Methyl-1H-Pyrazolo[4,3-C]Pyridin-7-yl)-1H-Pyrazole-4-Sulfonamide

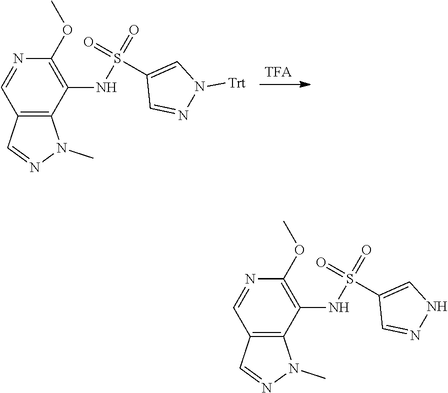

A solution of N-(6-methoxy-1-methyl-1H-pyrazolo[4,3-c]pyridin-7-yl)-1-trityl-1H-pyrazole-4-sulfonamide (142 mg, 0.258 mmol, 1 eq.) in DCM (1 mL) and TFA (2 mL) was stirred for 2 h at room temperature under a nitrogen atmosphere. The resulting mixture was concentrated under vacuum. The residue was dissolved with water (10 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 0% to 100% gradient in 20 min; detector: UV 254 nm. The resulting mixture was concentrated under vacuum. This resulted in N-(6-methoxy-1-methyl-1H-pyrazolo[4,3-c]pyridin-7-yl)-1H-pyrazole-4-sulfonamide (56 mg, 63.39%) as off-white solid. (ES, m/z): [M+H]⁺ 309.

### Example 236—Synthesis of 1-(4-Cyclopropylpyridin-2-yl)-N-(6-Methoxy-1-Methyl-1H-Pyrazolo[4,3-C]Pyridin-7-yl)-1H-Pyrazole-4-Sulfonamide (I-198)

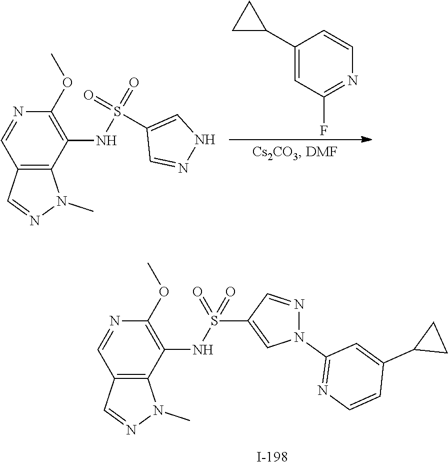 [SMILES: COc1ncc2cnn(C)c2c1NS(=O)(=O)c1cn[nH]c1 ; Fc1cc(C2CC2)ccn1 ; COc1ncc2cnn(C)c2c1NS(=O)(=O)c1cnn(-c2cc(C3CC3)ccn2)c1]

To a stirred solution of N-(6-methoxy-1-methyl-1H-pyrazolo[4,3-c]pyridin-7-yl)-1H-pyrazole-4-sulfonamide (56 mg, 0.182 mmol, 1 eq.) and 4-cyclopropyl-2-fluoropyridine (49.82 mg, 0.364 mmol, 2 eq.) in DMF (1 mL) was added Cs₂CO₃ (177.54 mg, 0.546 mmol, 3 eq.) in portions at 80° C. under a nitrogen atmosphere. The resulting mixture was stirred overnight at 80° C. under a nitrogen atmosphere. The reaction was quenched with water (10 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 1-(4-cyclopropylpyridin-2-yl)-N-(6-methoxy-1-methyl-1H-pyrazolo[4,3-c]pyridin-7-yl)-1H-pyrazole-4-sulfonamide (4.6 mg, 5.92%) as a white solid. (ES, m/z): [M+H]⁺=427; ¹H NMR: (400 MHz, DMSO-d₆) δ 0.90-0.93 (m, 2H), 1.13-1.18 (m, 2H), 2.12-2.14 (m, 1H), 3.42 (s, 3H), 4.21 (s, 3H), 7.14 (d, J=5.2, 1.7 Hz, 1H), 7.70 (s, 1H), 7.98 (s, 1H), 8.22 (s, 1H), 8.32 (d, J=4.8 Hz, 1H), 8.65 (s, 1H), 8.70 (s, 1H), 9.93 (s, 1H).

### Example 237—Synthesis of N-Methyl-N-(1-Methyl-1H-Indazol-7-yl)-1-(4-(Trifluoromethyl)Pyridin-2-yl)-1H-Pyrazole-4-Sulfonamide (I-199)

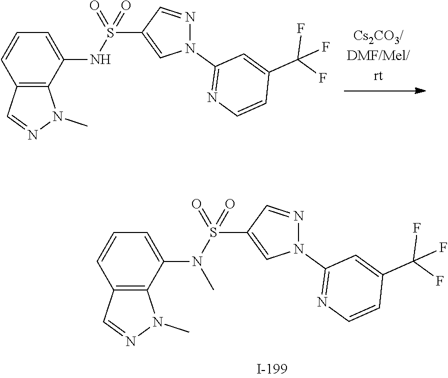 [SMILES: Cn1ncc2cccc(NS(=O)(=O)c3cnn(-c4cc(C(F)(F)F)ccn4)c3)c21 ; CN(c1cccc2cnn(C)c12)S(=O)(=O)c1cnn(-c2cc(C(F)(F)F)ccn2)c1]

A solution of N-(1-methyl-1H-indazol-7-yl)-1-(4-(trifluoromethyl)pyridin-2-yl)-1H-pyrazole-4-sulfonamide (150 mg, 0.355 mmol, 1 eq.), Mel (75.6 mg, 0.532 mmol, 1.50 eq.) and Cs₂CO₃ (231.4 mg, 0.710 mmol, 2.00 eq.) in DMF (5 mL) was stirred for 4 h at room temperature. The resulting mixture was diluted with water (15 mL). The resulting mixture was extracted with EtOAc (3×10 mL). The combined organic layers were washed with brine (15 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃⁺ 0.05% NH₃·H₂O), mobile phase B: ACN; flow rate: 60 mL/min; gradient: 42% B to 67% B in 7 min; wavelength: 220 nm; Rt(min): 7.75, to afford N-methyl-N-(1-methyl-1H-indazol-7-yl)-1-(4-(trifluoromethyl)pyridin-2-yl)-1H-pyrazole-4-sulfonamide (50.7 mg, 32.68%) as an off-white solid. (ES, m/z): [M+H]⁺ 437 ¹H NMR (DMSO-d₆, 400 MHz) δ 3.32 (s, 3H), 4.29 (s, 3H), 6.91 (d, J=7.2 Hz, 1H), 7.03 (t, J=7.6 Hz, 1H), 7.78 (d, J=7.6 Hz, 1H), 7.91 (d, J=4.8 Hz, 1H), 8.12 (s, 2H), 8.24 (s, 1H), 8.85 (d, J=4.8 Hz, 1H), 9.06 (s, 1H).

### Example 238—Synthesis of 1-(4-(1,1-Difluoroethyl)Pyridin-2-yl)-N-(1-Methyl-1H-Indazol-7-yl)-1H-Pyrazole-4-Sulfonamide (I-200)

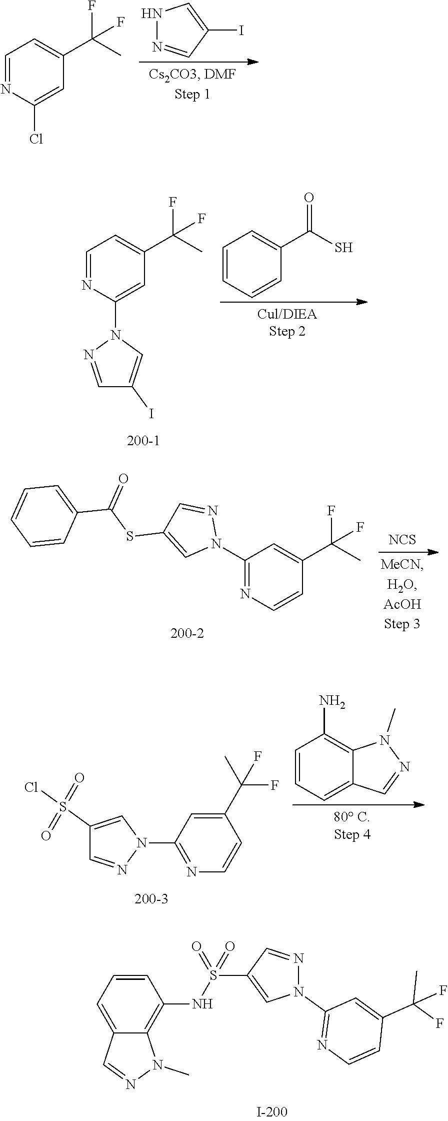

Step 1: Synthesis of 200-1. A solution of 2-chloro-4-(1,1-difluoroethyl)pyridine (16 g, 90.100 mmol, 1 eq.), Cs₂CO₃ (58.71 g, 180.200 mmol, 2 eq.) and 4-iodopyrazole (19.22 g, 99.110 mmol, 1.1 eq.) in DMF (200 mL) was stirred for 4 h at 100° C. under a nitrogen atmosphere. The resulting mixture was diluted with water (1 L). The aqueous layer was extracted with DCM (3×500 mL). The combined organic layers were dried over anhydrous Na₂SO₄. After filtration, the resulting mixture was concentrated under vacuum to give 4-(1,1-difluoroethyl)-2-(4-iodopyrazol-1-yl)pyridine (200-1, 30 g, 99.36%) as brown oil. The crude product was used in next step without any purification.

Step 2: Synthesis of 200-2. A solution of 4-(1,1-difluoroethyl)-2-(4-iodopyrazol-1-yl)pyridine (30 g, 89.527 mmol, 1 eq.), CuI (1.71 g, 8.953 mmol, 0.1 eq.), DIEA (23.14 g, 179.054 mmol, 2 eq.), 1,10-phenanthroline (3.23 g, 17.905 mmol, 0.2 eq.) and benzenecarbothioic acid (14.84 g, 107.432 mmol, 1.2 eq.) in toluene (800 mL) was stirred overnight at 110° C. under a nitrogen atmosphere. The reaction was concentrated under vacuum. The resulting mixture was diluted with water (500 mL) at room temperature. The aqueous layer was extracted with EtOAc (3×500 mL). The combined organic layers were dried over anhydrous Na₂SO₄. After filtration, the resulting mixture was concentrated under vacuum. The residue was purified by silica gel column chromatography and eluted with PE/EA (7:1) to afford ({1-[4-(1,1-difluoroethyl)pyridin-2-yl]pyrazol-4-yl}sulfanyl)(phenyl)methanone (30 g, 97.03%) as a pink solid.

Step 3: Synthesis 200-3. A solution of ({1-[4-(1,1-difluoroethyl)pyridin-2-yl]pyrazol-4-yl}sulfanyl)(phenyl)methanone (30 g, 86.863 mmol, 1 eq.) and NCS (34.80 g, 260.589 mmol, 3 eq.) in ACN (500 mL) and AcOH (50 mL) and H₂O (50 mL) was stirred for 1 h at room temperature. The reaction was concentrated under vacuum. The resulting mixture was diluted with water (500 mL) at room temperature. The aqueous layer was extracted with DCM (3×500 mL). The residue was purified by silica gel column chromatography, eluting with PE/EA (30:1) to afford 1-[4-(1,1-difluoroethyl)pyridin-2-yl]pyrazole-4-sulfonyl chloride (25 g, 93.54%) as a yellow oil.

Step 4: Synthesis of 1-[4-(1,1-difluoroethyl)pyridin-2-yl]-N-(1-methylindazol-7-yl)pyrazole-4-sulfonamide (I-200). A solution of 1-[4-(1,1-difluoroethyl)pyridin-2-yl]pyrazole-4-sulfonyl chloride (15 g, 48.749 mmol, 1 eq.) and 1-methylindazol-7-amine (7.17 g, 48.749 mmol, 1 eq.) in pyridine (300 mL) was stirred for 1 h at room temperature. The reaction solution was concentrated under vacuum. The resulting mixture was diluted with water (200 mL). The aqueous layer was extracted with CH₂Cl₂ (3×200 mL). The crude product was recrystallized from MeCN (20 mL) and the residue was purified by reversed-phase flash chromatography with the following conditions: column: C18 silica gel; mobile phase: MeCN in water (0.1% TFA), 0% to 100% gradient in 40 min; detector: UV 254 nm, to afford 1-[4-(1,1-difluoroethyl)pyridin-2-yl]-N-(1-methylindazol-7-yl)pyrazole-4-sulfonamide (10 g, 49.03%) as a white solid. (ES, m/z): [M+H]⁺ 419; ¹H NMR: 1H NMR (400 MHz, DMSO-d) δ 2.07 (t, J=8.8 Hz, 3H), 4.31 (s, 3H), 6.74 (d, J=8.8 Hz, 1H), 6.98 (d, J=7.6 Hz, 1H), 7.67-7.73 (m, 2H), 8.09-8.10 (m, 3H), 8.68 (d, J=4.8 Hz, 1H), 8.86 (s, 1H), 10.08 (s, 1H).

### Example 239—Synthesis of 1-(4-(3-Fluorooxetan-3-yl)Pyridin-2-yl)-N-(1-Methyl-1H-Indazol-7-yl)-1H-Pyrazole-4-Sulfonamide (I-201)

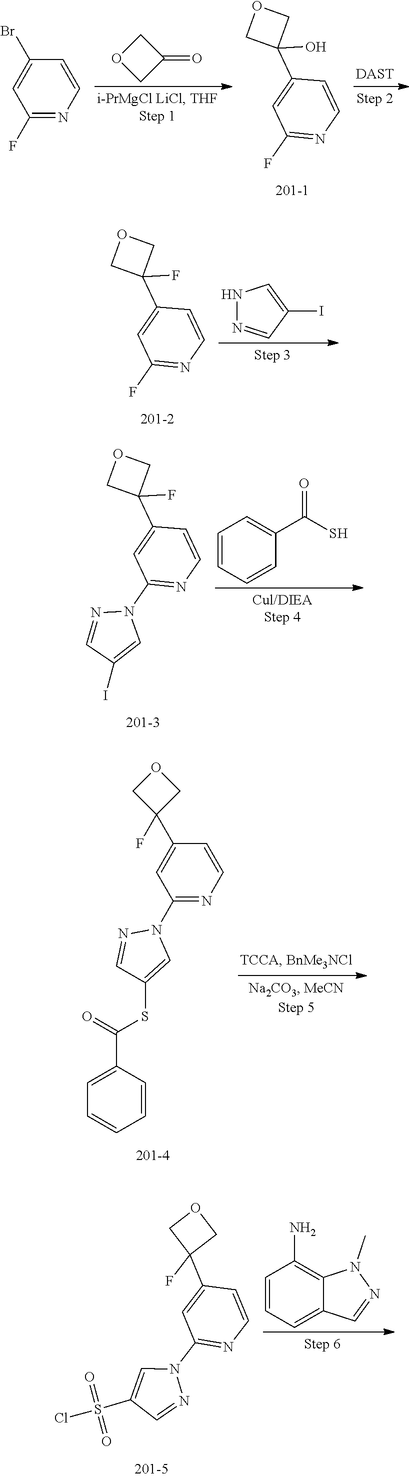

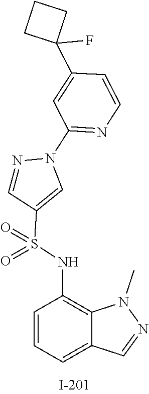 [SMILES: Cn1ncc2cccc(NS(=O)(=O)c3cnn(-c4cc(C5(F)CCC5)ccn4)c3)c21]

Step 1: Synthesis of 201-1. To a stirred solution of 4-bromo-2-fluoropyridine (30.00 g, 170.466 mmol, 1 eq.) in THE (300.00 mL) was added i-PrMgCl—LiCl (1.30 M in THF, 74.27 g, 511.398 mmol, 3 eq.) dropwise at −78° C. under a nitrogen atmosphere. The resulting mixture was stirred for 2 h at −78° C. under a nitrogen atmosphere. To the above mixture was added oxetan-3-one (36.85 g, 511.398 mmol, 3 eq.) dropwise over 10 min at −78° C. The resulting mixture was stirred for an additional 1 h at room temperature. The mixture was acidified to pH 6 with conc. HCl. The resulting mixture was extracted with EtOAc (3×300 mL). The combined organic layers were washed with brine (1 L) and dried over anhydrous Na₂SO₄. After filtration, the filtrate was concentrated under reduced pressure. This resulted in 3-(2-fluoropyridin-4-yl)oxetan-3-ol (25.00 g, 78.03%) as a pink solid.

Step 2: Synthesis of 201-2. To a stirred solution of 3-(2-fluoropyridin-4-yl)oxetan-3-ol (18.00 g, 106.411 mmol, 1 eq.) in DCM (180 mL) was added DAST (34.30 g, 212.822 mmol, 2 eq.) dropwise at 0° C. under a nitrogen atmosphere. The resulting mixture was stirred for 2 h at 0° C. under a nitrogen atmosphere. The mixture was diluted with H₂O (500 mL) and extracted with EtOAc (3×200 mL). The combined organic layers were washed with brine (500 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 60% gradient in 10 min; detector: UV 254 nm, to afford 2-fluoro-4-(3-fluorooxetan-3-yl)pyridine (12.00 g, 63.98%) as an off-white solid.

Step 3: Synthesis of 201-3. A mixture of 2-fluoro-4-(3-fluorooxetan-3-yl)pyridine (11.00 g, 64.272 mmol, 1 eq.), 4-iodo-1H-pyrazole (14.96 g, 77.126 mmol, 1.2 eq.) and Cs₂CO₃ (62.82 g, 192.816 mmol, 3 eq.) in DMF (100 mL) was stirred for 2 h at 80° C. The mixture was diluted with H₂O (500 mL). The resulting mixture was extracted with EtOAc (3×200 mL). The combined organic layers were washed with brine (500 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford 4-(3-fluorooxetan-3-yl)-2-(4-iodo-1H-pyrazol-1-yl)pyridine (12.00 g, 52.53%) as an off-white solid.

Step 4: Synthesis of 201-4. A mixture of 4-(3-fluorooxetan-3-yl)-2-(4-iodo-1H-pyrazol-1-yl)pyridine (13.00 g, 37.668 mmol, 1 eq.), benzothioic acid (6.25 g, 45.202 mmol, 1.2 eq.), 1,10-phenanthroline (1.36 g, 7.534 mmol, 0.2 eq.), DIEA (9.74 g, 75.336 mmol, 2 eq.) and CuI (0.72 g, 3.767 mmol, 0.1 eq.) in toluene (20 mL) was stirred for 16 h at 110° C. under a nitrogen atmosphere. The mixture was concentrated under reduced pressure. The resulting mixture was diluted with H₂O (200 mL). The resulting mixture was extracted with EtOAc (3×200 mL). The combined organic layers were washed with brine (500 mL) and dried over anhydrous Na₂SO₄. After filtration, the filtrate was concentrated under reduced pressure. The residue was purified by silica gel column chromatography, eluting with PE/EA (4:1) to afford S-(1-(4-(3-fluorooxetan-3-yl)pyridin-2-yl)-1H-pyrazol-4-yl) benzothioate (12.00 g, 87.04%) as an off-white solid.

Step 5. Synthesis of 201-5. A solution of trichloro-1,3,5-triazinane-2,4,6-trione (2.35 g, 10.129 mmol, 1.2 eq.) in MeCN (30 mL) was treated with benzyltrimethylazanium chloride (5.33 g, 28.699 mmol, 3.4 eq.) for 30 min at room temperature under a nitrogen atmosphere followed by the addition of S-(1-(4-(3-fluorooxetan-3-yl)pyridin-2-yl)-1H-pyrazol-4-yl) benzothioate (3.00 g, 8.441 mmol, 1 eq.) dropwise at 0° C. To the above mixture was added Na₂CO₃ (0.89 g, 8.441 mmol, 1 eq.) dropwise at 0° C. The resulting mixture was stirred for 1 h at 0° C. under a nitrogen atmosphere. The reaction was quenched with water/ice. The resulting mixture was extracted with EtOAc (3×40 mL). The combined organic layers were washed with brine (50 mL) and dried over anhydrous Na₂SO₄. After filtration, the filtrate was concentrated under reduced pressure to afford 1-(4-(3-fluorooxetan-3-yl)pyridin-2-yl)-1H-pyrazole-4-sulfonyl chloride (2.00 g, 72.41%) as an off-white solid.

Step 6. Synthesis of 1-(4-(3-fluorooxetan-3-yl)pyridin-2-yl)-N-(1-methyl-1H-indazol-7-yl)-1H-pyrazole-4-sulfonamide (I-201). A mixture of 1-(4-(3-fluorooxetan-3-yl)pyridin-2-yl)-1H-pyrazole-4-sulfonyl chloride (1.50 g, 4.721 mmol, 1 eq.) and 1-methyl-1H-indol-7-amine (0.69 g, 4.721 mmol, 1 eq.) in pyridine (15 mL) was stirred for 30 min at 80° C. The mixture was concentrated under reduced pressure. The resulting mixture was diluted with H₂O (50 mL). The resulting mixture was extracted with EtOAc (3×50 mL). The combined organic layers were washed with brine (100 mL) and dried over anhydrous Na₂SO₄. After filtration, the filtrate was concentrated under reduced pressure. The crude product was purified by Prep-SFC with the following conditions: column: YMC-Actus Triart Diol-HILIC 3*25 cm, 5 μm; mobile phase A: CO₂, mobile phase B: MeOH (0.1% 2 M NH₃-MeOH); flow rate: 75 mL/min; gradient: isocratic 22% B, to afford 1-(4-(3-fluorooxetan-3-yl)pyridin-2-yl)-N-(1-methyl-1H-indazol-7-yl)-1H-pyrazole-4-sulfonamide (0.4209 g, 20.39%) as an off-white solid. (ES, m/z): [M+H]⁺=429; ¹H NMR: (400 MHz, DMSO-d₆) δ 4.29 (s, 3H), 4.90-5.08 (m, 4H), 6.71 (d, J=7.2 Hz, 1H), 6.97 (t, J=7.6 Hz, 1H), 7.69 (d, J=5.2 Hz, 2H), 8.08-8.10 (m, 3H), 8.64 (d, J=5.2 Hz, 1H), 8.84 (s, 1H), 10.06 (s, 1H).

### Example 240—Synthesis of 1-(4-(3-Methyoxyoxetan-3-yl)Pyridin-2-yl)-N-(1-Methyl-1H-Indazol-7-yl)-1H-Pyrazole-4-Sulfonamide (I-202)

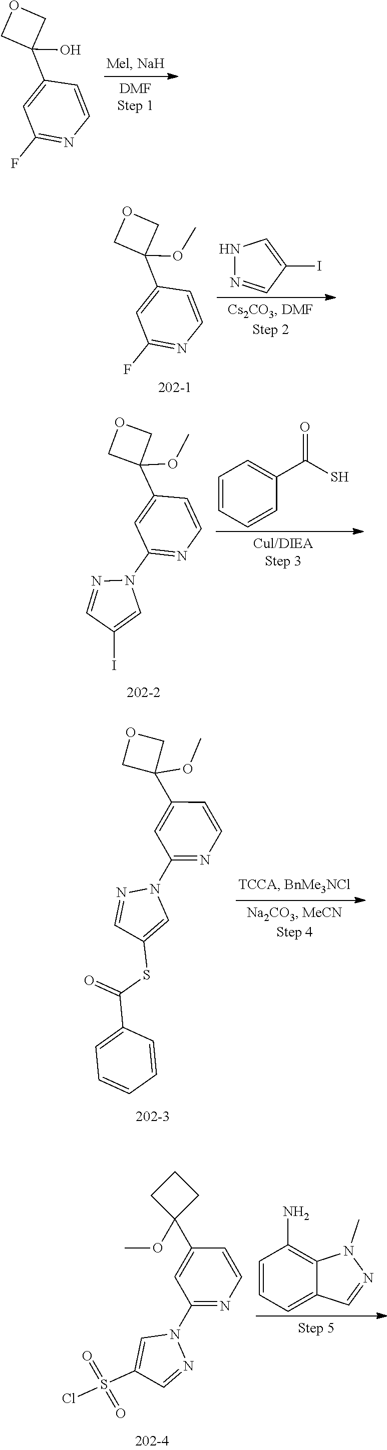

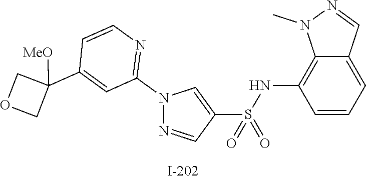 [SMILES: COC1(c2ccnc(-n3cc(S(=O)(=O)Nc4cccc5cnn(C)c45)cn3)c2)COC1]

Step 1: Synthesis of 202-1. To a stirred mixture of 3-(2-fluoropyridin-4-yl)oxetan-3-ol (20.00 g, 118.235 mmol, 1 eq.) and NaH (4.26 g, 177.352 mmol, 1.5 eq.) in DMF (200 mL) was added methyl iodide (20.14 g, 141.882 mmol, 1.20 eq.) dropwise at 0° C. under a nitrogen atmosphere. The resulting mixture was stirred for 2 h at 0° C. under a nitrogen atmosphere. The reaction was quenched with water (200 mL). The resulting mixture was extracted with EtOAc (3×150 mL). The combined organic layers were washed with brine (100 mL) and dried over anhydrous Na₂SO₄. After filtration, the filtrate was concentrated under reduced pressure to afford 2-fluoro-4-(3-methoxyoxetan-3-yl)pyridine (17.00 g, 76.21%) as an off-white oil.

Step 2. Synthesis of 202-2. A mixture of 2-fluoro-4-(3-methoxyoxetan-3-yl)pyridine (15.00 g, 81.886 mmol, 1 eq.), 4-iodo-1H-pyrazole (19.06 g, 98.263 mmol, 1.2 eq.) and Cs₂CO₃ (80.04 g, 245.658 mmol, 3 eq.) in DMF (150 mL) was stirred for 2 h at 80° C. The reaction was quenched with water (200 mL). The resulting mixture was extracted with EtOAc (3×500 mL). The combined organic layers were washed with brine (500 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 60% to 70% gradient in 10 min; detector: UV 254 nm, to afford 2-(4-iodo-1H-pyrazol-1-yl)-4-(3-methoxyoxetan-3-yl)pyridine (20.00 g, 66.40%) as an off-white solid.

Step 3: Synthesis of 202-3. A mixture of 2-(4-iodo-1H-pyrazol-1-yl)-4-(3-methoxyoxetan-3-yl)pyridine (18.00 g, 50.399 mmol, 1 eq.), 1,10-phenanthroline (1.82 g, 10.080 mmol, 0.2 eq.), DIEA (13.03 g, 100.798 mmol, 2 eq.), benzothioic acid (8.36 g, 60.479 mmol, 1.2 eq.) and CuI (0.96 g, 5.040 mmol, 0.1 eq.) in toluene (180 mL) was stirred for 16 h at 110° C. under a nitrogen atmosphere. The reaction was quenched with water (200 mL) and extracted with EtOAc (3×300 mL). The combined organic layers were washed with brine (500 mL) and dried over anhydrous Na₂SO₄. After filtration, the filtrate was concentrated under reduced pressure. The residue was purified by silica gel column chromatography and eluted with PE/EA (3:1), to afford S-(1-(4-(3-methoxyoxetan-3-yl)pyridin-2-yl)-1H-pyrazol-4-yl) benzothioate (15.00 g, 78.66%) as an off-white solid.

Step 4: Synthesis of 202-4. A solution of trichloro-1,3,5-triazinane-2,4,6-trione (1.52 g, 6.532 mmol, 1.2 eq.) in MeCN (20 mL) was treated with benzyltrimethylazanium chloride (3.44 g, 18.506 mmol, 3.4 eq.) for 30 min at room temperature under a nitrogen atmosphere followed by the addition of S-(1-(4-(3-methoxyoxetan-3-yl)pyridin-2-yl)-1H-pyrazol-4-yl) benzothioate (2.00 g, 5.443 mmol, 1 eq.) dropwise at 0° C. To the above mixture was added Na₂CO₃ (0.58 g, 5.443 mmol, 1 eq.) at 0° C. The resulting mixture was stirred for 1 h at 0° C. under a nitrogen atmosphere. The reaction was quenched with water (20 mL). The resulting mixture was extracted with EtOAc (3×15 mL). The combined organic layers were washed with brine (50 mL) and dried over anhydrous Na₂SO₄. After filtration, the filtrate was concentrated under reduced pressure, to afford 1-(4-(3-methoxyoxetan-3-yl)pyridin-2-yl)-1H-pyrazole-4-sulfonyl chloride (1.00 g, 54.09%) as an off-white solid.

Step 5: Synthesis of 1-[4-(3-methoxyoxetan-3-yl)pyridin-2-yl]-N-(1-methylindazol-7-yl)pyrazole-4-sulfonamide1-(4-(3-methoxyoxetan-3-yl)pyridin-2-yl)-N-(1-methyl-1H-indazol-7-yl)-1H-pyrazole-4-sulfonamide (I-202). A mixture of 1-(4-(3-methoxyoxetan-3-yl)pyridin-2-yl)-1H-pyrazole-4-sulfonyl chloride (3 g, 9.098 mmol, 1 eq.) and 1-methyl-1H-indazol-7-amine (1.34 g, 9.098 mmol, 1 eq.) in pyridine (30 mL) was stirred for 30 min at 80° C. The reaction was quenched with water (30 mL). The resulting mixture was extracted with EtOAc (3×30 mL). The combined organic layers were washed with brine (50 mL) and dried over anhydrous Na₂SO₄. After filtration, the filtrate was concentrated under reduced pressure. The residue was purified by silica gel column chromatography and eluted with PE/EA (1:1), to afford 1-[4-(3-methoxyoxetan-3-yl)pyridin-2-yl]-N-(1-methylindazol-7-yl)pyrazole-4-sulfonamide1-(4-(3-methoxyoxetan-3-yl)pyridin-2-yl)-N-(1-methyl-1H-indazol-7-yl)-1H-pyrazole-4-sulfonamide (2.8474 g, 70.13%) as an off-white solid. (ES, m/z): [M+H]⁺=441; ¹H NMR: (400 MHz, DMSO-d₆) δ 3.19 (s, 3H), 4.29 (s, 3H), 4.74 (d, J=7.6 Hz, 2H), 4.88 (d, J=7.6 Hz, 2H), 6.72 (dd, J=7.2, 0.8 Hz, 1H), 6.98 (t, J=7.6 Hz, 1H), 7.64 (dd, J=5.2, 1.6 Hz, 1H), 7.71 (dd, J=8.0, 0.8 Hz, 1H), 8.05-8.10 (m, 3H), 8.60 (d, J=5.2 Hz, 1H), 8.84 (s, 1H), 10.05 (s, 1H).

### Example 241—Synthesis of N-(1-Ethyl-1H-Indazol-7-yl)-1-(4-(Trifluoromethyl)Pyridin-2-yl)-1H-Pyrazole-4-Sulfonamide (I-203)

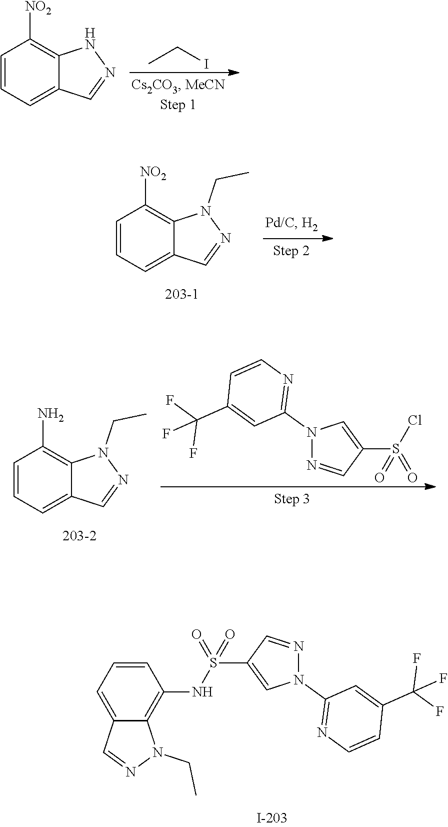

Step 1: Synthesis of 203-1. To a stirred solution of 7-nitroindazole (1 g, 6.130 mmol, 1 eq.) and Cs₂CO₃ (5.99 g, 18.390 mmol, 3 eq.) in MeCN (10 mL) was added ethyl iodide (0.98 mL, 12.260 mmol, 2 eq.) dropwise at room temperature under a nitrogen atmosphere. The resulting mixture was stirred overnight at room temperature under a nitrogen atmosphere. The resulting mixture was diluted with CH₂Cl₂ (10 mL). The combined organic layers were washed with water (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 60% gradient in 20 min; detector: UV 254 nm. This resulted in 203-1 (400 mg, 30.72%) as a yellow solid.

Step 2: Synthesis of 203-2. A mixture of 1-ethyl-7-nitroindazole (200 mg, 1.046 mmol, 1 eq.) and Pd/C (111.32 mg, 1.046 mmol, 1 eq.) in MeOH (5 mL) was stirred for 2 h at room temperature under a hydrogen atmosphere. The resulting mixture was filtered, the filter cake was washed with MeOH (3×10 mL). The filtrate was concentrated under reduced pressure. This resulted in 203-2 (190 mg, 90.13%) as a brown solid.

Step 3: Synthesis of N-(1-ethyl-1H-indazol-7-yl)-1-(4-(trifluoromethyl)pyridin-2-yl)-1H-pyrazole-4-sulfonamide (I-203). To a stirred solution of 1-ethylindazol-7-amine (150 mg, 0.930 mmol, 1 eq.) and 1-[4-(trifluoromethyl)pyridin-2-yl]pyrazole-4-sulfonyl chloride (434.99 mg, 1.395 mmol, 1.5 eq.) in pyridine (6 mL) was added DMAP (34.10 mg, 0.279 mmol, 0.3 eq.) at room temperature under a nitrogen atmosphere. The resulting mixture was stirred for 16 h at 80° C. The resulting mixture was diluted with CH₂Cl₂ (20 mL). The combined organic layers were washed with water (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 70% gradient in 20 min; detector: UV 254 nm. The crude product (150 mg) was purified by prep-HPLC with the following conditions: column: XBridge Prep OBD C18 column, 30*150 mm, 5 μm; mobile phase A: water (10 mmol/L NH₄CO₃, 0.05% NH₃·H₂O), mobile phase B: ACN; flow rate: 60 mL/min; gradient: 23% B to 50% B in 7 min; Rt(min): 7.85) to afford N-(1-ethyl-1H-indazol-7-yl)-1-(4-(trifluoromethyl)pyridin-2-yl)-1H-pyrazole-4-sulfonamide (93.6 mg, 22.13%) as an off-white solid. (ES, m/z): [M+H]=437; ¹H NMR: ¹H NMR (400 MHz, DMSO-d₆) δ 1.41 (t, J=7.2 Hz, 3H), 4.77 (q, J=7.2 Hz, 2H), 6.70 (dd, J=7.6, 0.8 Hz, 1H), 6.97 (t, J=7.6, 1H), 7.71 (dd, J=8.0, 1.2 Hz, 1H), 7.89 (dd, J=5.2, 1.2 Hz, 1H), 8.11 (s, 2H), 8.21 (s, 1H), 8.83 (d, J=5.2 Hz, 1H), 8.88 (s, 1H), 10.12 (s, 1H).

### Example 242—Synthesis of 1-(5-(Hydroxymethyl)-4-Methylpyridin-2-yl)-N-(1-Methyl-1H-Indazol-7-yl)-1H-Pyrazole-4-Sulfonamide (I-204)

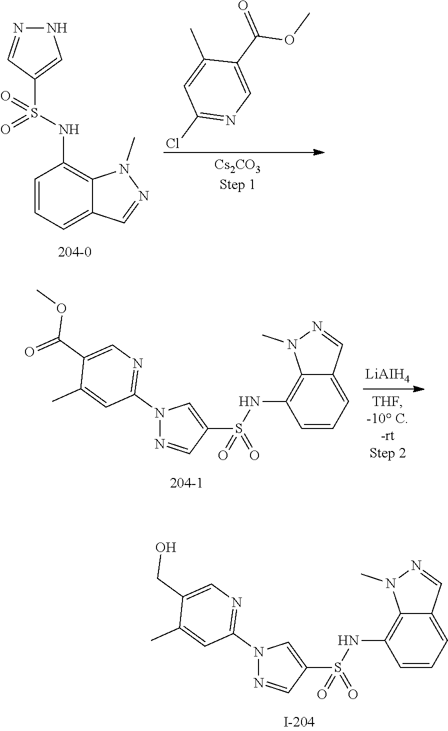

Step 1: Synthesis of 204-1. To a stirred mixture of 204-0 (200 mg, 0.721 mmol, 1 eq.) and methyl 6-chloro-4-methylpyridine-3-carboxylate (200 mg, 1.078 mmol, 1.49 eq.) in DMSO (6 mL) was added Cs₂CO₃ (705 mg, 2.164 mmol, 3.00 eq.) in portions at room temperature under a nitrogen atmosphere. The resulting mixture was stirred for 5 h at 140° C. The resulting mixture was diluted with water (10 mL). The resulting mixture was extracted with EtOAc (3×10 mL). The combined organic layers were washed with brine (5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 20% to 50% gradient in 20 min; detector: UV 254 nm. This resulted in 204-1 (100 mg, 32.51%) as an off-white solid.

Step 2: Synthesis of 1-(5-(hydroxymethyl)-4-methylpyridin-2-yl)-N-(1-methyl-1H-indazol-7-yl)-1H-pyrazole-4-sulfonamide (I-204). A solution of 204-1 (40 mg, 0.094 mmol, 1 eq.) in THF (6 mL) was treated with LiAlH₄ (20 mg, 0.527 mmol, 5.62 eq.) for 2 h at −20° C. under a nitrogen atmosphere. The reaction was quenched by the addition of sat. NH₄Cl (aq.) (1 mL) at room temperature. The resulting mixture was diluted with water (5 mL). The resulting mixture was extracted with EtOAc (3×10 mL). The combined organic layers were washed with brine (5 mL) and dried over anhydrous Na₂SO₄. After filtration, the filtrate was concentrated under reduced pressure. The crude product (38 mg) was purified by prep-HPLC with the following conditions: column: XBridge Shield RP18 OBD column, 30*150 mm, 5 μm; mobile phase A: water (10 mmol/L NH₄CO₃+0.05% NH₃H₂O), mobile phase B: ACN; flow rate: 60 mL/min; gradient: 9% B to 33% B in 7 min) to afford 1-(5-(hydroxymethyl)-4-methylpyridin-2-yl)-N-(1-methyl-1H-indazol-7-yl)-1H-pyrazole-4-sulfonamide (17.5 mg, 46.59%) as an off-white solid. (ES, m/z): [M+H]⁺ 399; ¹H NMR: (400 MHz, DMSO-d₆) δ 2.41 (s, 3H), 4.29 (s, 3H), 4.58 (d, J=5.6 Hz, 2H), 5.30 (t, J=5.6 Hz, 1H), 6.70 (d, J=7.6 Hz, 1H), 6.95 (t, J=5.6 Hz, 1H), 7.61 (s, 1H), 7.81 (s, 1H), 7.97 (s, 1H), 8.05 (s, 1H), 8.36 (s, 1H), 8.75 (s, 1H), 9.99 (s, 1H).

### Example 243—Synthesis of 1-(4-(Difluoromethyl)Pyridin-2-yl)-N-(1-Methyl-1H-Indazol-7-yl)-1H-Pyrazole-4-Sulfonamide (I-205)

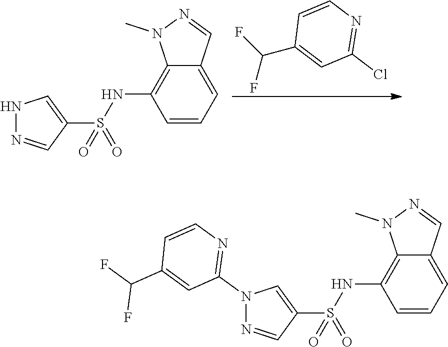

A solution of N-(1-methylindazol-7-yl)-1H-pyrazole-4-sulfonamide (100 mg, 0.361 mmol, 1 eq.), 2-chloro-4-(difluoromethyl)pyridine (117.96 mg, 0.722 mmol, 2 eq.), CuI (34.34 mg, 0.180 mmol, 0.5 eq.), N₁,N₂-dimethylcyclohexane-1,2-diamine (51.30 mg, 0.361 mmol, 1 eq.) and Cs₂CO₃ (352.49 mg, 1.083 mmol, 3 eq.) in DMF (1.5 mL) was stirred for 16 h at 140° C. under a nitrogen atmosphere. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-[4-(difluoromethyl)pyridin-2-yl]-N-(1-methylindazol-7-yl)pyrazole-4-sulfonamide (24.8 mg, 16.68%) as a white solid. (ES, m/z): [M+H]⁺ 405; ¹H NMR: (400 MHz, DMSO-d₆) δ 4.29 (s, 3H), 6.73 (dd, J=7.2, 0.8 Hz, 1H), 6.96 (d, J=7.6 Hz, 1H), 7.25 (t, J=14.8 Hz, 1H), 7.67-7.69 (m, 2H), 8.07-8.13 (m, 3H), 8.70 (d, J=5.2 Hz, 1H), 8.83 (s, 1H), 10.06 (s, 1H).

### Example 244—Synthesis of 1-(4-(Difluoromethyl)Pyridin-2-yl)-N-(1-Ethyl-1H-Indazol-7-yl)-1H-Pyrazole-4-Sulfonamide (I-206)

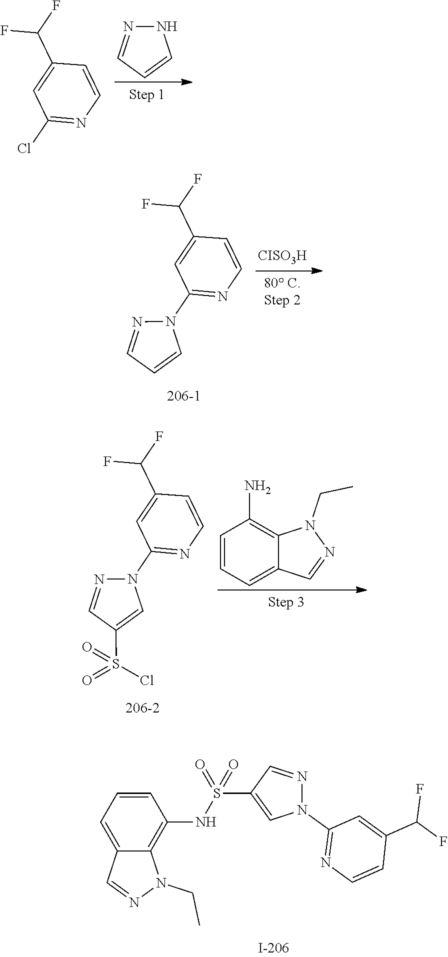

Step 1: Synthesis of 206-1. A solution of 2-chloro-4-(difluoromethyl)pyridine (650 mg, 3.974 mmol, 1 eq.), pyrazole (811.70 mg, 11.922 mmol, 3 eq.) and t-BuOK (1337.91 mg, 11.922 mmol, 3 eq.) in ACN (8 mL) was stirred for 16 h at 80° C. The resulting mixture was quenched with H₂O (20 ml) and extracted with EtOAc (3×20 mL). The combined organic layers were dried over anhydrous Na₂SO₄. After filtration, the resulting mixture was concentrated under vacuum. The residue was purified by reversed-phase flash chromatography with the following conditions: column: C18 silica gel; mobile phase: ACN in water, 10% to 80% gradient in 30 min; detector: UV 254 nm. The resulting mixture was concentrated under reduced pressure. This resulted in 206-1 (650 mg, 83.80%) as a yellow oil.

Step 2: Synthesis of 206-2. Into a 100 ml round bottom flask were added 206-1 (600 mg, 3.074 mmol, 1 eq.) and chlorosulfonic acid (8 mL) at 0° C. Then the resulting mixture was stirred overnight at 80° C. The resulting mixture was added into dilute hydrochloric acid (6 g ice and 4 mL conc HCl) very carefully. The resulting mixture was extracted with DCM (3×50 mL) and dried over anhydrous Na₂SO₄. The organic was concentrated under reduced pressure. This resulted in 206-2 (640 mg, crude) as a brown solid.

Step 3: Synthesis of 1-(4-(difluoromethyl)pyridin-2-yl)-N-(1-ethyl-1H-indazol-7-yl)-1H-pyrazole-4-sulfonamide (I-206). A mixture of 206-2 (364 mg, 1.239 mmol, 2 eq.) and 1-ethylindazol-7-amine (99.91 mg, 0.620 mmol, 1 eq.) in pyridine (8 mL) was stirred for 2 hours at room temperature. The resulting mixture was concentrated under reduced pressure. The residue was purified by reversed-phase flash chromatography with the following conditions: column: C18 silica gel; mobile phase: water in ACN, 0% to 100% gradient in 25 min; detector: UV 254 nm. The resulting mixture was concentrated under reduced pressure. This resulted in I-205 (92.3 mg, 35.33%) as an off-white solid. (ES, m/z): [M+H]⁺ 419; ¹H NMR: (400 MHz, DMSO-d₆) δ 1.41 (t, J=7.2 Hz, 3H), 4.77 (q, J=7.2 Hz, 2H), 6.71 (d, J=7.2 Hz, 1H), 6.97 (t, J=7.6 Hz, 1H), 7.25 (t, J=14.8 Hz, 1H), 7.67-7.71 (m, 2H), 8.08 (s, 1H), 8.12 (s, 1H), 8.14 (s, 1H), 8.71 (d, J=5.2 Hz, 1H), 8.84 (s, 1H), 10.09 (s, 1H).

### Example 245—Synthesis of 1-(4-(Difluoromethyl)Pyridin-2-yl)-N-(1-(Methyl-D3)-1H-Indazol-7-yl)-1H-Pyrazole-4-Sulfonamide (I-207)

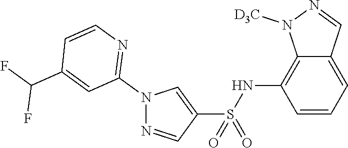 [SMILES: [2H]C([2H])([2H])n1ncc2cccc(NS(=O)(=O)c3cnn(-c4cc(C(F)F)ccn4)c3)c21]

A solution of 1-(4-(difluoromethyl)pyridin-2-yl)-1H-pyrazole-4-sulfonyl chloride (140 mg, 0.477 mmol, 1.5 eq.) and 1-(methyl-d3)-1H-indazol-7-amine (47.74 mg, 0.318 mmol, 1 eq.) in pyridine (5 mL) was stirred for 2 hours at room temperature. The resulting mixture was concentrated under reduced pressure. The residue was purified by reversed-phase flash chromatography with the following conditions: column: C18 silica gel; mobile phase: MeCN in water, 10% to 100% gradient in 30 min; detector: UV 254 nm. The resulting mixture was concentrated under reduced pressure. This resulted in I-207 (51.4 mg, 39.39%) as an off-white solid. (ES, m/z): [M+H]⁺ 407; 1H NMR (400 MHz, DMSO-d₆) δ 6.73 (d, J=7.2 Hz, 1H), 6.97 (t, J=7.6 Hz, 1H), 7.25 (t, J=14.8 Hz, 1H), 7.67-7.70 (m, 2H), 8.07 (s, 1H), 8.09 (s, 1H), 8.13 (s, 1H), 8.70 (d, J=4.8 Hz, 1H), 8.84 (s, 1H), 10.06 (s, 1H).

### Example 246—Synthesis of 1-(6-Methoxy-4-(Trifluoromethyl) Pyridin-2-yl)-N-(1-Methyl-1H-Indazol-7-yl)-1H-Pyrazole-4-Sulfonamide (I-208)

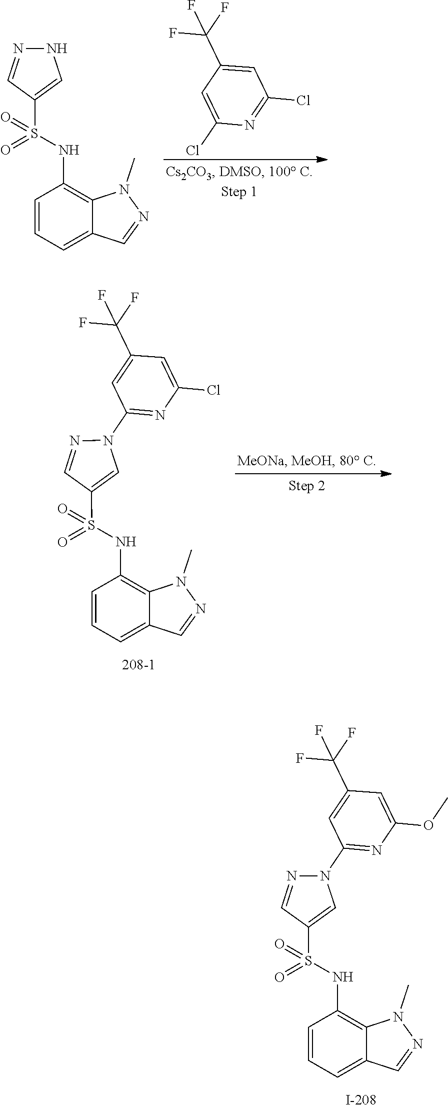 [SMILES: Cn1ncc2cccc(NS(=O)(=O)c3cn[nH]c3)c21 ; FC(F)(F)c1cc(Cl)nc(Cl)c1 ; Cn1ncc2cccc(NS(=O)(=O)c3cnn(-c4cc(C(F)(F)F)cc(Cl)n4)c3)c21 ; COc1cc(C(F)(F)F)cc(-n2cc(S(=O)(=O)Nc3cccc4cnn(C)c34)cn2)n1]

Step 1: Synthesis of 208-1. A solution of N-(1-methylindazol-7-yl)-1H-pyrazole-4-sulfonamide (150 mg, 0.541 mmol, 1 eq.), 2,6-dichloro-4-(trifluoromethyl)pyridine (175.25 mg, 0.812 mmol, 1.5 eq.) and Cs₂CO₃ (528.74 mg, 1.623 mmol, 3 eq.) in DMSO (5 mL) was stirred overnight at 80° C. under a nitrogen atmosphere. The resulting mixture was extracted with EtOAc (2×50 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 208-1 (180 mg, 72.84%) as an off-white solid.

Step 2: Synthesis of 1-(6-methoxy-4-(trifluoromethyl)pyridine-2-yl)-N-(1-methyl-1H-indazol-7-yl)-1H-pyrazole-4-sulfonamide (I-208). A solution of 208-1 and MeONa (29.56 mg, 0.545 mmol, 5 eq.) in MeOH (3 mL) was stirred for 4 h at 80° C. under a nitrogen atmosphere. The mixture was concentrated under reduced pressure. The residue was purified by reversed-phase flash chromatography with the following conditions: column: C18 silica gel; mobile phase: MeCN in water (0.1% NH₃·H₂O), 60% to 70% gradient in 10 min; detector: UV 254 nm. This resulted in I-208 (25.7 mg, 51.49%) as a pink solid. (ES, m/z): [M+H]⁺=453; ¹H NMR (400 MHz, DMSO-d₆) δ 4.01 (s, 3H), 4.29 (s, 3H), 6.72 (d, J=7.2 Hz, 1H), 6.99 (dd, J=8.0, 7.2 Hz, 1H), 7.32 (s, 1H), 7.71-7.72 (m, 2H), 8.10 (s, 2H), 8.90 (d, J=0.8 Hz, 1H), 10.09 (s, 1H).

### Example 247—Synthesis of 1-(4-Chloropyridin-2-yl)-N-(1-(Methyl-D3)-1H-Indazol-7-yl)-1H-Pyrazole-4-Sulfonamide (I-209)

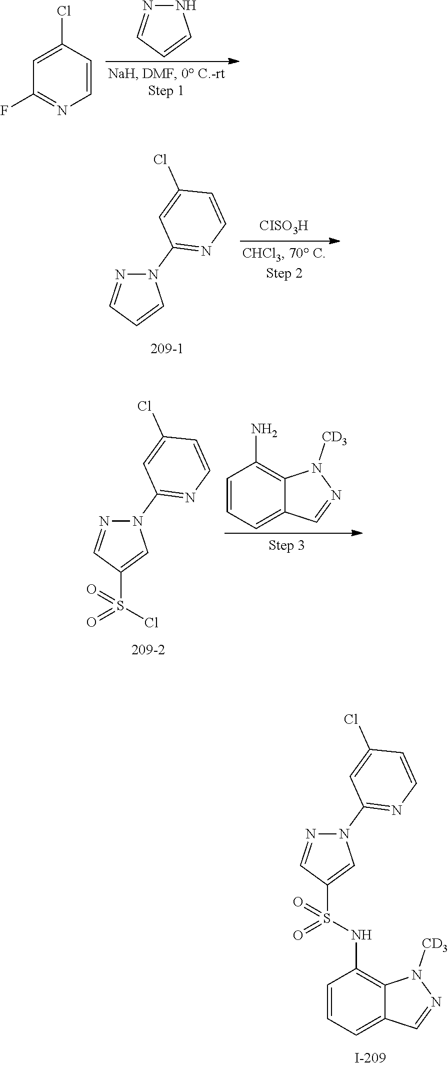

Step 1: Synthesis of 209-1. A solution of 4-chloro-2-fluoropyridine (1 g, 7.603 mmol, 1 eq.), pyrazole (0.78 g, 11.404 mmol, 1.5 eq.) and Cs₂CO₃ (7.43 g, 22.809 mmol, 3 eq.) in DMF (10 mL) was stirred for 16 h at room temperature under an air atmosphere. The resulting mixture was diluted with CH₂Cl₂ (50 mL). The combined organic layers were washed with water (3×50 mL) and dried over anhydrous Na₂SO₄. After filtration, the filtrate was concentrated under reduced pressure. This resulted in 4-chloro-2-(pyrazol-1-yl)pyridine (1.5 g, 98.86%) as a white solid.

Step 2: Synthesis of 209-2. Into a 10 mL round-bottom flask were added 4-chloro-2-(pyrazol-1-yl)pyridine (500 mg, 2.784 mmol, 1 eq.) and chlorosulfonic acid (3 mL, 45.134 mmol, 16.21 eq.) at 0° C. The resulting mixture was stirred for 16 h at 70° C. under an air atmosphere. The reaction was quenched with water/ice at 0° C. The aqueous layer was extracted with CH₂Cl₂ (3×30 mL). The combined organic layers were washed with brine (50 mL) and dried over anhydrous Na₂SO₄. The resulting mixture was concentrated under reduced pressure. This resulted in 1-(4-chloropyridin-2-yl)pyrazole-4-sulfonyl chloride (630 mg, 74.05%) as a yellow solid.

Step 3: Synthesis of 1-(4-chloropyridin-2-yl)-N-(1-(methyl-d3)-1H-indazol-7-yl)-1H-pyrazole-4-sulfonamide (I-209). A mixture of 1-(4-chloropyridin-2-yl)pyrazole-4-sulfonyl chloride (100 mg, 0.360 mmol, 1 eq.) and 1-(methyl-d3)-1H-indazol-7-amine (81.01 mg, 0.540 mmol, 1.5 eq.) in pyridine (1.5 mL) was stirred for 2 h at room temperature under an air atmosphere. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(4-chloropyridin-2-yl)-N-(1-(methyl-d3)-1H-indazol-7-yl)-1H-pyrazole-4-sulfonamide (33.7 mg, 23.65%) as a white solid. (ES, m/z): [M+H]+ 392; ¹H NMR: (400 MHz, DMSO-d₆) δ 6.71-6.73 (m, 1H), 6.96 (t, J=7.6 Hz, 1H), 7.62-7.67 (m, 2H), 8.03-8.07 (m, 3H), 8.52 (d, J=5.6 Hz, 1H), 8.79 (s, 1H), 10.06 (s, 1H).

### Example 248—Synthesis of N-(3-Methylimidazo[1,5-A]Pyridin-5-yl)-1-(4-(Trifluoromethyl)Pyridin-2-yl)-1H-Pyrazole-4-Sulfonamide (I-210)

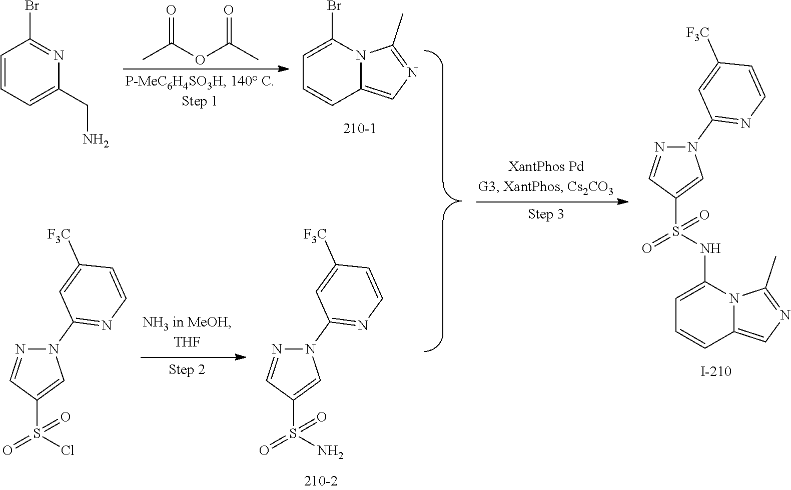

Step 1: Synthesis of 210-1. To a stirred solution of 1-(6-bromopyridin-2-yl) methanamine (2 g, 10.693 mmol, 1 eq.) in acetic anhydride (5 mL) was added p-toluenesulfonic acid (2.035 g, 11.818 mmol, 1.11 eq.) in portions at 0° C. under a nitrogen atmosphere. The resulting mixture was stirred for 2 h at 140° C. under a nitrogen atmosphere. The resulting mixture was extracted with EtOAc (2×100 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. The resulting mixture was concentrated under reduced pressure. This resulted in 210-1 (2 g, 88.62%) as a brown solid.

Step 2: Synthesis of 210-2. To a stirred solution of 1-[4-(trifluoromethyl) pyridin-2-yl]pyrazole-4-sulfonyl chloride (500 mg, 1.604 mmol, 1 eq.) in THE (5 mL) was added NH3 in MeOH (35.23 mL, 35.224 mmol, 21.96 eq.) dropwise at room temperature under a nitrogen atmosphere. The resulting mixture was stirred for 2 h at room temperature under a nitrogen atmosphere. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. The resulting mixture was concentrated under reduced pressure. This resulted in 210-2 (300 mg, 63.99%) as a white solid.

Step 3: Synthesis of N-(3-methylimidazo[1,5-a]pyridin-5-yl)-1-(4-(trifluoromethyl) pyridin-2-yl)-1H-pyrazole-4-sulfonamide (I-210). To a stirred mixture of 210-1(166.15 mg, 0.569 mmol, 1.2 eq.) and 210-2 (100 mg, 0.474 mmol, 1 eq.) in dioxane (15 mL) were added XantPhos (54.83 mg, 0.095 mmol, 0.2 eq.) and Xantphos Pd G3 (89.87 mg, 0.095 mmol, 0.2 eq.) and Cs₂CO₃ (463.11 mg, 1.422 mmol, 3 eq.) in portions at room temperature under a nitrogen atmosphere. The resulting mixture was stirred for 5 h at 90° C. under a nitrogen atmosphere. The resulting mixture was extracted with EtOAc (2×50 mL). The combined organic layers were washed with brine (2×10 mL) and dried over anhydrous Na₂SO₄. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in the title compound as a reddish brown solid. (ES, m/z): [M+H]⁺ 423; ¹H NMR: (400 MHz, DMSO-d₆) δ 3.20 (s, 3H), 6.11 (d, J=7.6 Hz, 1H), 6.77-6.81 (m, 2H), 7.55 (s, 1H), 7.79 (d, J=5.2, 1H), 8.09 (s, 1H), 8.10 (s, 1H), 8.77 (s, 1H), 8.79 (s, 1H).

### Example 249—Synthesis of 1-(5-(1-Hydroxyethyl)-4-Methylpyridin-2-yl)-N-(1-Methyl-1H-Indazol-7-yl)-1H-Pyrazole-4-Sulfonamide (I-211)

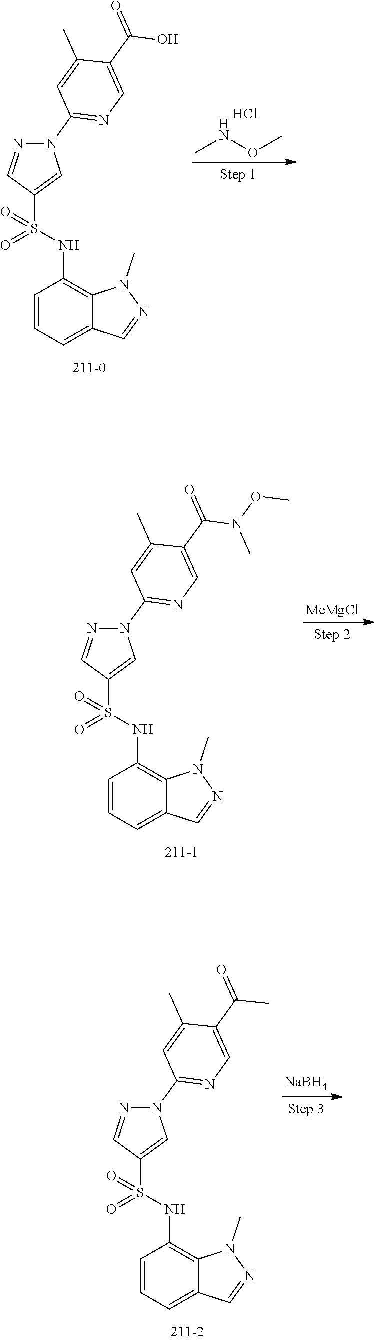

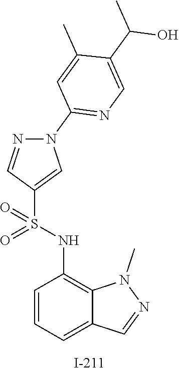 [SMILES: Cc1cc(-n2cc(S(=O)(=O)Nc3cccc4cnn(C)c34)cn2)ncc1C(C)O]

Step 1: Synthesis of 211-1. To a stirred solution of 211-0 (80 mg, 0.194 mmol, 1 eq.) and N,O-dimethylhydroxylamine hydrochloride (22.70 mg, 0.233 mmol, 1.2 eq.) in DMF (15 mL) were added EDCI (55.78 mg, 0.291 mmol, 1.5 eq.) DIEA (10 ml) and HOBT (39.32 mg, 0.291 mmol, 1.5 eq.) dropwise at room temperature under a nitrogen atmosphere. The resulting mixture was stirred for 2 h at room temperature. The resulting mixture was quenched with NH₄Cl (sat.) (50 mL) and extracted with EtOAc (5×20 mL). The combined organic layers were washed with brine (10 mL) and dried over anhydrous Na₂SO₄. After filtration, the filtrate was concentrated under reduced pressure. This resulted in 211-1 (30 mg, 33.95%) as a yellow oil.

Step 2: Synthesis of 211-2. To a stirred solution of 211-1 (30 mg, 0.066 mmol, 1 eq.) in THF (5 mL) were added methylmagnesium chloride (9.85 mg, 0.132 mmol, 2 eq.) dropwise at 0° C. under a nitrogen atmosphere. The resulting mixture was stirred for an additional 2 h at 0° C. to room temperature. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. This resulted in 211-2 (25 mg, 92.48%) as a yellow oil.

Step 3: Synthesis of 1-(5-(1-hydroxyethyl)-4-methylpyridin-2-yl)-N-(1-methyl-1H-indazol-7-yl)-1H-pyrazole-4-sulfonamide (I-211). To a stirred solution of 211-2 (25 mg, 0.061 mmol, 1 eq.) in THE (10 mg) were added NaBH₄ (4.61 mg, 0.122 mmol, 2 eq.) in portions at 0° C. under a nitrogen atmosphere. The resulting mixture was stirred for 2 h at room temperature. The resulting mixture was quenched with water (50 mL) and extracted with CH₂Cl₂ (5×20 mL). The combined organic layers were washed with brine (10 mL) and dried over anhydrous Na₂SO₄. After filtration, the filtrate was concentrated under reduced pressure. The crude product (25 mg) was purified by prep-HPLC with the following conditions: column: YMC-Actus Triart C18 ExRS, 30*150 mm, 5 μm; mobile phase A: water (10 mmol/L NH₄CO₃+0.05% NH₃H₂O), mobile phase B: ACN; flow rate: 60 mL/min; gradient: 10% B to 35% B in 7 min; wavelength: 220 nm; Rt(min): 8.17. This resulted in I-211 (6.5 mg, 25.77%) as a white solid. (ES, m/z): [M+H]=412; ¹H NMR: 1H NMR (400 MHz, DMSO-d₆) δ 1.38 (d, J=6.4 Hz, 3H), 2.43 (s, 3H), 4.29 (s, 3H), 4.29 (s, 1H), 4.96-5.02 (m, 1H), 5.37 (d, J=4.4 Hz, 1H), 6.70 (d, J=7.2 Hz, 1H), 6.95 (t, J=7.6 Hz, 1H), 7.66 (s, 1H), 7.77 (s, 1H), 7.97 (s, 1H), 8.07 (s, 1H), 8.46 (s, 1H), 8.75 (s, 1H), 10.00 (s, 1H).

### Example 250—Synthesis of 1-(4-(Hydroxymethyl)Pyridin-2-yl)-N-(1-Methyl-1H-Indazol-7-yl)-1H-Pyrazole-4-Sulfonamide (I-212)

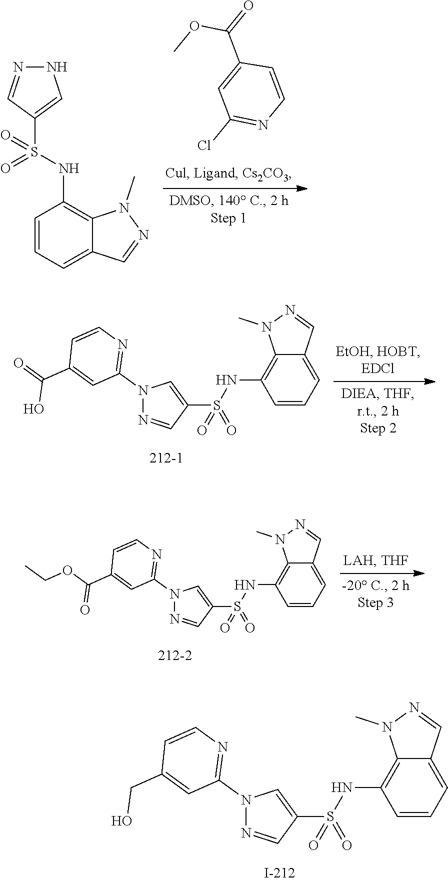 [SMILES: Cn1ncc2cccc(NS(=O)(=O)c3cn[nH]c3)c21 ; COC(=O)c1ccnc(Cl)c1 ; Cn1ncc2cccc(NS(=O)(=O)c3cnn(-c4cc(C(=O)O)ccn4)c3)c21 ; CCOC(=O)c1ccnc(-n2cc(S(=O)(=O)Nc3cccc4cnn(C)c34)cn2)c1 ; Cn1ncc2cccc(NS(=O)(=O)c3cnn(-c4cc(CO)ccn4)c3)c21]

Step 1: Synthesis of 212-1. A mixture of N-(1-methylindazol-7-yl)-1H-pyrazole-4-sulfonamide (200 mg, 0.721 mmol, 1 eq.), methyl 6-chloropyridine-3-carboxylate (178 mg, 1.037 mmol, 1.44 eq.), CuI (120 mg, 0.630 mmol, 0.87 eq.), (1R,2R)—N1,N2-dimethylcyclohexane-1,2-diamine (20 mg, 0.141 mmol, 0.19 eq.) and Cs₂CO₃ (470 mg, 1.443 mmol, 2.00 eq.) in DMSO (8 mL) was stirred for 2 h at 140° C. under a nitrogen atmosphere. The resulting mixture was quenched with water (10 mL) and extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeOH in water (0.1% TFA), 10% to 50% gradient in 10 min; detector: UV 254 nm. This resulted in 212-1 (180 mg, 62.64%) as an off-white solid.

Step 2: Synthesis of 212-2. The mixture of 212-1 (100 mg, 0.251 mmol, 1 eq.), EtOH (1 mL, 17.213 mmol, 68.58 eq.), HOBt (50 mg, 0.370 mmol, 1.47 eq.), EDCI (60 mg, 0.313 mmol, 1.25 eq.) and DIEA (43 mg, 0.333 mmol, 1.33 eq.) in THE (5 mL) was stirred for 2 h at room temperature under a nitrogen atmosphere. The residue was quenched with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by Prep-TLC (PE/EA 3:1) to afford 212-2 (60 mg, 56.05%) as an off-white solid.

Step 3: Synthesis of 1-(4-(hydroxymethyl)pyridin-2-yl)-N-(1-methyl-1H-indazol-7-yl)-1H-pyrazole-4-sulfonamide (I-212). To a solution of 212-2 (50 mg, 0.117 mmol, 1 eq.) in THF (3 mL) was added LiAlH₄ (30 mg, 0.791 mmol, 6.74 eq.) at −20° C. under a nitrogen atmosphere. The resulting mixture was stirred for 2 h at −20° C. to room temperature and then quenched with sat. NH₄Cl (aq.). The resulting mixture was extracted with CH₂Cl₂ (5×10 mL). The combined organic layers were washed with brine (4 mL) and dried over anhydrous Na₂SO₄. After filtration, the filtrate was concentrated under reduced pressure. The crude product (60 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 18% B to 42% B in 7 min, to afford I-212 (5.4 mg, 11.86%) as an off-white solid. (ES, m/z): [M+H]⁺ 385; ¹H NMR (400 MHz, DMSO-d₆) δ 4.28 (s, 3H), 4.66 (d, J=5.6 Hz, 2H), 5.63 (t, J=5.6 Hz, 1H), 6.71 (d, J=7.2 Hz, 1H), 6.98 (t, J=7.6 Hz, 1H), 7.39 (d, J=4.8 Hz, 1H), 7.70 (d, J=8.4 Hz, 1H), 7.98 (s, 1H), 8.01 (s, 1H), 8.09 (s, 1H), 8.43 (d, J=4.8 Hz, 1H), 8.79 (s, 1H), 10.02 (s, 1H).

### Example 251—Synthesis of 1-(4-(1-Hydroxyethyl)Pyridin-2-yl)-N-(1-Methyl-1H-Indazol-7-yl)-1H-Pyrazole-4-Sulfonamide (I-213)

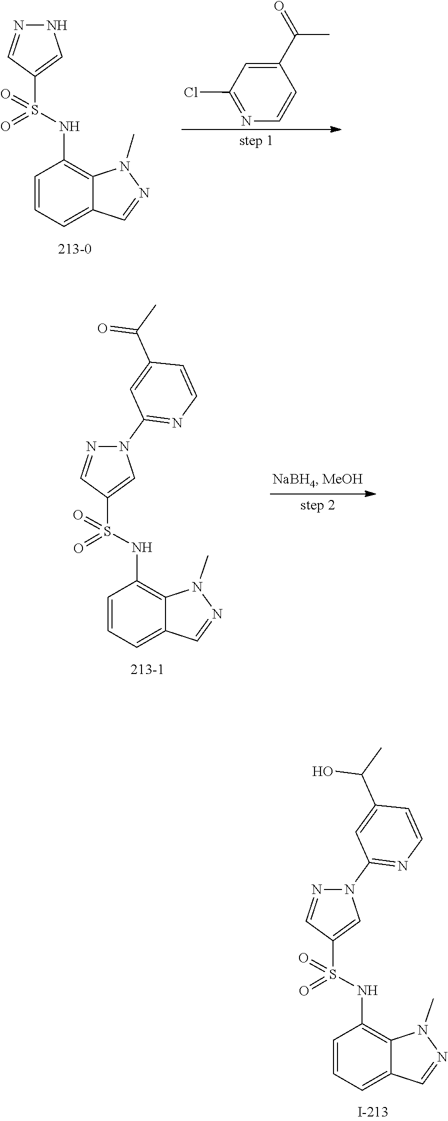

Step 1: Synthesis of 213-1. A mixture of 213-0 (150 mg, 0.541 mmol, 1 eq.), 1-(2-chloropyridin-4-yl)378yridine (126.24 mg, 0.811 mmol, 1.50 eq.) and Cs₂CO₃ (371.88 mg, 1.142 mmol, 2.11 eq.) in DMF (4 mL) was stirred for 2 h at 140° C. under a nitrogen atmosphere. The resulting mixture was diluted with water (40 mL). The resulting mixture was extracted with EtOAc (3×100 mL). The combined organic layers were washed with brine (10 mL) and dried over anhydrous Na₂SO₄. After filtration, the filtrate was concentrated under reduced pressure. The residue was purified by Prep-TLC (EA/ethyl ether 1:1) to afford 213-1 (70 mg, 32.64%) as a yellow oil.

Step 2: Synthesis of 1-(4-(1-hydroxyethyl)pyridine-2-yl)-N-(1-methyl-1H-indazol-7-yl)-1H-pyrazole-4-sulfonamide (I-213). A mixture of 1-(5-acetylpyridin-2-yl)-N-(1-methylindazol-7-yl)pyrazole-4-sulfonamide (60 mg, 0.151 mmol, 1 eq.) and NaBH4 (30 mg, 0.793 mmol, 5.24 eq.) in MeOH (2 mL) was stirred for 2 h at −20° C. under a nitrogen atmosphere. The reaction was quenched by the addition of sat. NH4Cl (aq.) (2 mL) at room temperature. The resulting mixture was extracted with EtOAc (3×20 mL). The combined organic layers were washed with brine (2 mL) and dried over anhydrous Na₂SO₄. After filtration, the filtrate was concentrated under reduced pressure. The crude product (80 mg) was purified by prep-HPLC with the following conditions: column: Xbridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 10% B to 32% B in 7 min; wavelength: 220 nm; Rt(min): 7.5) to afford I-213 (16.2 mg, 26.86%) as an off-white solid. (ES, m/z): [M+H]⁺=399; ¹H NMR: 1H NMR (400 MHz, DMSO-d₆) δ 1.38 (d, J=6.8 Hz, 3H), 4.29 (s, 3H), 4.85-4.87 (m, 1H), 5.61 (d, J=4.8 Hz, 1H), 6.72 (d, J=7.2 Hz, 1H), 6.96 (t, J=7.2 Hz, 1H), 7.42 (d, J=4.4 Hz, 1H), 7.63 (d, J=8.0 Hz, 1H), 7.99 (s, 1H), 8.00 (s, 1H), 8.06 (s, 1H), 8.43 (d, J=4.8 Hz, 1H), 8.77 (s, 1H), 10.00 (s, 1H).

### Example 252—Synthesis of 1-(4-(2-Hydroxypropan-2-yl)Pyridin-2-yl)-N-(1-Methyl-1H-Indazol-7-yl)-1H-Pyrazole-4-Sulfonamide (I-214)

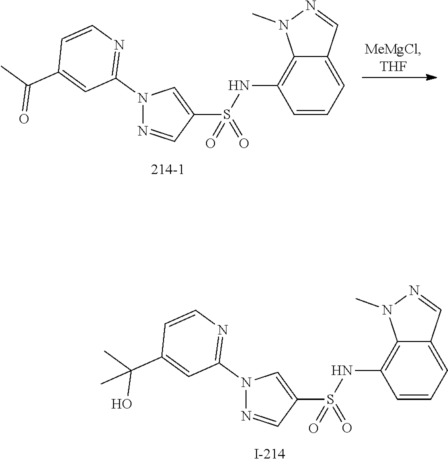 [SMILES: CC(=O)c1ccnc(-n2cc(S(=O)(=O)Nc3cccc4cnn(C)c34)cn2)c1 ; Cn1ncc2cccc(NS(=O)(=O)c3cnn(-c4cc(C(C)(C)O)ccn4)c3)c21]

A mixture of 214-1 (70 mg, 0.177 mmol, 1 eq.) and bromo(methyl)magnesium (0.2 mL) in THE (2 mL) was stirred for 2 h at 0° C. under a nitrogen atmosphere. The reaction was quenched by the addition of sat. NH₄Cl (aq.) (2 mL) at room temperature. The resulting mixture was extracted with EtOAc (3×20 mL). The combined organic layers were washed with brine (2 mL) and dried over anhydrous Na₂SO₄. After filtration, the filtrate was concentrated under reduced pressure. The crude product (120 mg) was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 10% B to 34% B in 7 min; wavelength: 254 nm/220 nm; Rt(min): 7.95) to afford 1-214 (16.8 mg, 23.07%) as an off-white solid. (ES, m/z): [M+H]⁺=413; ¹H NMR: 1H NMR (400 MHz, DMSO-d₆) δ 1.47 (s, 6H), 4.29 (s, 3H), 5.49 (s, 1H), 6.71 (d, J=7.2 Hz, 1H), 6.97 (t, J=7.6 Hz, 1H), 7.52 (dd, J=5.6, 1.6 Hz, 1H), 8.00 (s, 1H), 8.08 (s, 1H), 8.10 (s, 1H), 8.43 (d, J=5.2 Hz, 1H), 8.78 (s, 1H), 10.02 (s, 1H).

### Example 253—Synthesis of N-(1-Methyl-1H-Indazol-7-yl)-1-(6-OXO-4-(Trifluoromethyl)-1,6-Dihydropyridin-2-yl)-1H-Pyrazole-4-Sulfonamide (I-215)

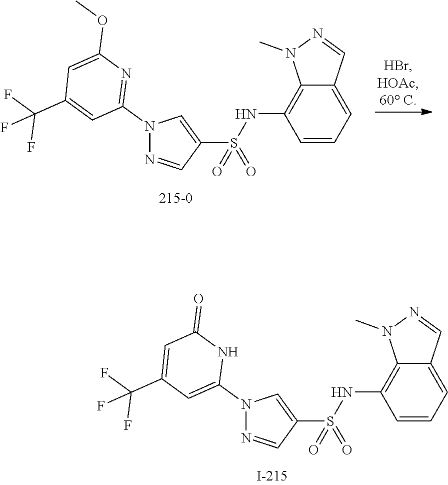 [SMILES: COc1cc(C(F)(F)F)cc(-n2cc(S(=O)(=O)Nc3cccc4cnn(C)c34)cn2)n1 ; Cn1ncc2cccc(NS(=O)(=O)c3cnn(-c4cc(C(F)(F)F)cc(=O)[nH]4)c3)c21]

A solution of 215-0 (55 mg, 0.122 mmol, 1 eq.) in HBr/AcOH (2 mL) was stirred overnight at 80° C. under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 60% to 70% gradient in 10 min; detector: UV 254 nm. This resulted in I-215 (38.3 mg, 71.00%) as an off-white solid. (ES, m/z): [M+H]⁺ 439; ¹H NMR (400 MHz, DMSO-d₆) δ 4.28 (s, 3H), 6.70 (d, J=7.2 Hz, 1H), 6.99 (t, J=7.6 Hz, 1H), 7.04 (s, 1H), 7.60 (s, 1H), 7.72 (d, J=7.6 Hz, 1H), 8.06 (s, 1H), 8.10 (s, 1H), 8.67 (s, 1H), 10.09 (s, 1H), 12.27 (s, 1H).

### Example 254—Synthesis of 1-(4-Fluoropyridin-2-yl)-N-(1-Methyl-1H-Indazol-7-yl)-1H-Pyrazole-4-Sulfonamide (I-216)

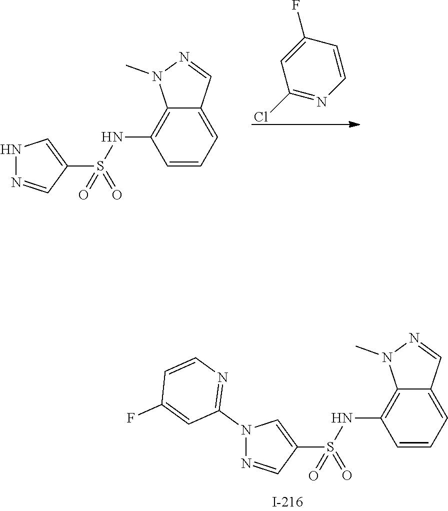 [SMILES: Cn1ncc2cccc(NS(=O)(=O)c3cn[nH]c3)c21 ; Fc1ccnc(Cl)c1 ; Cn1ncc2cccc(NS(=O)(=O)c3cnn(-c4cc(F)ccn4)c3)c21]

A solution of N-(1-methylindazol-7-yl)-1H-pyrazole-4-sulfonamide (100 mg, 0.361 mmol, 1 eq.), 2-chloro-4-fluoropyridine (71.15 mg, 0.541 mmol, 1.5 eq.), CuI (34.34 mg, 0.180 mmol, 0.5 eq.), N1,N2-dimethylcyclohexane-1,2-diamine (51.30 mg, 0.361 mmol, 1 eq.) and Na₂CO₃ (114.66 mg, 1.083 mmol, 3 eq.) in DMF (1.5 mL) was stirred for 16 h at 110° C. under a nitrogen atmosphere. The reaction was quenched with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(4-fluoropyridin-2-yl)-N-(1-methylindazol-7-yl)pyrazole-4-sulfonamide (4.6 mg, 3.26%) as a white solid. (ES, m/z): [M+H]⁺ 373; ¹H NMR: (400 MHz, DMSO-d₆) δ 4.29 (s, 3H), 6.73 (d, J=7.2 Hz, 1H), 6.95 (t, J=7.6 Hz, 1H), 7.42-7.45 (m, 1H), 7.61 (d, J=6.8 Hz, 1H), 7.81 (dd, J=5.6, 1.6 Hz, 1H), 8.04 (s, 2H), 8.57 (dd, J=8.4, 1.6 Hz, 1H), 8.78 (s, 1H), 10.07 (s, 1H).

### Example 255—Synthesis of 1-(4-Chloropyridin-2-yl)-N-(1-Ethyl-1H-Indazol-7-yl)-1H-Pyrazole-4-Sulfonamide (I-217)

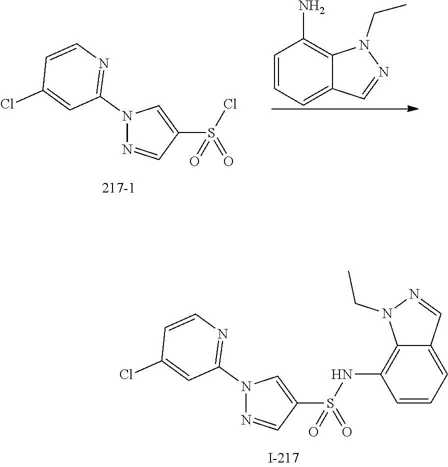 [SMILES: O=S(=O)(Cl)c1cnn(-c2cc(Cl)ccn2)c1 ; CCn1ncc2cccc(N)c21 ; CCn1ncc2cccc(NS(=O)(=O)c3cnn(-c4cc(Cl)ccn4)c3)c21]

A mixture of 1-(4-chloropyridin-2-yl)pyrazole-4-sulfonyl chloride (100 mg, 0.360 mmol, 1 eq.) and 1-ethylindazol-7-amine (86.95 mg, 0.540 mmol, 1.5 eq.) in pyridine (1.5 mL) was stirred for 2 h at room temperature under an air atmosphere. The reaction was quenched with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(4-chloropyridin-2-yl)-N-(1-ethylindazol-7-yl)pyrazole-4-sulfonamide (32 mg, 22.07%) as a white solid. (ES, m/z): [M+H]⁺ 402; ¹H NMR: (400 MHz, DMSO-d₆) δ 1.41 (t, J=7.2 Hz, 3H), 4.77 (q, J=7.2 Hz, 2H), 6.69 (dd, J=7.2 Hz, 1H), 6.97 (t, J=7.6 Hz, 1H), 7.64 (dd, J=5.6, 2.0 Hz, 1H), 7.71 (d, J=8.0 Hz, 1H), 8.04-8.07 (m, 2H), 8.13 (s, 1H), 8.52 (d, J=5.2 Hz, 1H), 8.81 (s, 1H), 10.10 (s, 1H).

### Example 256—Synthesis of 1-(2-Chloropyridin-4-yl)-N-(3-Fluoro-1-Methyl-1H-Indazol-7-yl)-1H-Pyrazole-4-Sulfonamide (I-218)

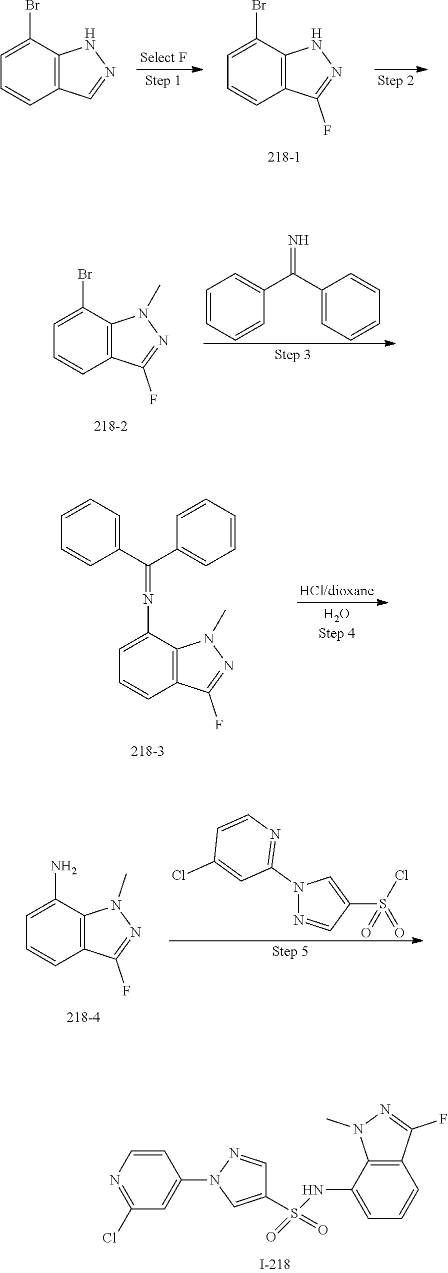

Step 1: Synthesis of 218-1. A solution of 7-bromo-1H-indazole (4 g, 20.301 mmol, 1.00 eq.) and Selectfluor (10.79 g, 30.451 mmol, 1.50 eq.) in MeCN (50 mL) was stirred overnight at 70° C. under a nitrogen atmosphere. The resulting mixture was concentrated under reduced pressure. The residue was purified by silica gel column chromatography, eluting with PE/EA (12:1) to afford 7-bromo-3-fluoro-1H-indazole (1.2 g, 27.49%) as a light yellow solid.

Step 2: Synthesis of 218-2. To a stirred solution of 7-bromo-3-fluoro-1H-indazole (500 mg, 2.325 mmol, 1.00 eq.) in THF (5 mL) were added t-BuONa (670.4 mg, 6.975 mmol, 3.00 eq.) dropwise at 0° C. under a nitrogen atmosphere. The resulting mixture was stirred for 30 min at room temperature under a nitrogen atmosphere. To the above mixture was added CH₃I (495.08 mg, 3.488 mmol, 1.50 eq.) dropwise over 1 min at room temperature. The resulting mixture was stirred for 1 h at room temperature under a nitrogen atmosphere. The reaction was quenched by the addition of sat. NH₄Cl (aq.) (10 mL) at room temperature. The resulting mixture was extracted with EtOAc (3×10 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (12:1) to afford 7-bromo-3-fluoro-1-methylindazole (250 mg, 32.86%) as a light yellow solid.

Step 3: Synthesis of 218-3. A solution of 7-bromo-3-fluoro-1-methylindazole (130 mg, 0.568 mmol, 1.00 eq.), Pd₂(dba)₃ (103.9 mg, 0.114 mmol, 0.20 eq.), XantPhos (82.1 mg, 0.142 mmol, 0.25 eq.), Cs₂CO₃ (554.7 mg, 1.704 mmol, 3.00 eq.) and diphenylmethanimine (123.4 mg, 0.682 mmol, 1.20 eq.) in dioxane (2 mL) was stirred for 2 h at 80° C. under a nitrogen atmosphere. The resulting mixture was diluted with water (10 mL). The resulting mixture was extracted with EtOAc (3×10 mL). The combined organic layers were washed with brine (10 mL) and dried over anhydrous Na₂SO₄. After filtration, the filtrate was concentrated under reduced pressure. The residue was purified by silica gel column chromatography, eluting with PE/EA (5:1) to afford N-(3-fluoro-1-methyl-1H-indazol-7-yl)-1,1-diphenylmethanimine (120 mg, crude) as a light yellow solid.

Step 4: Synthesis of 218-4. A solution of N-(3-fluoro-1-methyl-1H-indazol-7-yl)-1,1-diphenylmethanimine (130 mg, 0.395 mmol, 1.00 eq.) in HCl/dioxane (6M, 4 mL) and H₂O (4 mL) was stirred for 30 min at room temperature under a nitrogen atmosphere. The mixture was basified to pH 8 with saturated NaHCO₃ (aq.). The resulting mixture was extracted with EtOAc (3×10 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (5:1) to afford 3-fluoro-1-methyl-1H-indazol-7-amine (60 mg, cuede) as a light yellow solid.

Step 5: Synthesis of 1-(2-chloropyridin-4-yl)-N-(3-fluoro-1-methyl-1H-indazol-7-yl)-1H-pyrazole-4-sulfonamide (I-218). A solution of 3-fluoro-1-methyl-1H-indazol-7-amine (50 mg, 0.303 mmol, 1.00 eq.) and 1-(2-chloropyridin-4-yl)-1H-pyrazole-4-sulfonyl chloride (92.6 mg, 0.333 mmol, 1.10 eq.) in pyridine (5 mL) was stirred for 2 h at 80° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 45% B to 60% B in 7 min; wavelength: 220 nm; Rt(min): 6.83, to afford 1-(2-chloropyridin-4-yl)-N-(3-fluoro-1-methyl-1H-indazol-7-yl)-1H-pyrazole-4-sulfonamide (27 mg, 21.81%) as an off-white solid. (ES, m/z): [M+H]⁺ 407; ¹H NMR: (400 MHz, DMSO-d₆) δ 4.15 (s, 3H), 6.81 (d, J=7.2 Hz, 1H), 7.03 (t, J=7.6 Hz, 1H), 7.56-7.69 (m, 2H), 7.93-8.13 (m, 2H), 8.52 (d, J=5.2 Hz, 1H), 8.81 (s, 1H), 10.15 (s, 1H).

### Example 257—Synthesis of N-(3-Fluoro-1-Methyl-1H-Indazol-7-yl)-1-(4-(Trifluoromethyl)Pyridin-2-yl)-1H-Pyrazole-4-Sulfonamide (I-219)

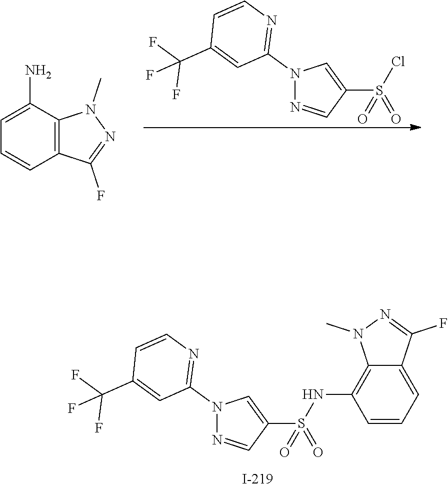 [SMILES: Cn1nc(F)c2cccc(N)c21 ; O=S(=O)(Cl)c1cnn(-c2cc(C(F)(F)F)ccn2)c1 ; Cn1nc(F)c2cccc(NS(=O)(=O)c3cnn(-c4cc(C(F)(F)F)ccn4)c3)c21]

A solution of 3-fluoro-1-methyl-1H-indazol-7-amine (50.0 mg, 0.303 mmol, 1.00 eq.) and 1-(4-(trifluoromethyl)pyridin-2-yl)-1H-pyrazole-4-sulfonyl chloride (103.7 mg, 0.333 mmol, 1.00 eq.) in pyridine (5 mL) was stirred for 2 h at 80° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 27% B to 53% B in 7 min; wavelength: 254 nm/220 nm; Rt(min): 7.77 to yield N-(3-fluoro-1-methyl-1H-indazol-7-yl)-1-(4-(trifluoromethyl)pyridin-2-yl)-1H-pyrazole-4-sulfonamide (27.8 mg, 20.39%) as an off-white solid. (ES, m/z): [M+H]⁺ 441; ¹H NMR: (400 MHz, DMSO-d₆) δ 4.16 (s, 3H), 6.83 (d, J=7.2 Hz, 1H), 7.02 (t, J=7.6 Hz, 1H), 7.60 (d, J=8.4 Hz, 1H), 7.88 (d, J=4.4 Hz, 1H), 8.10 (s, 1H), 8.19 (s, 1H), 8.81 (d, J=5.2 Hz, 1H), 8.87 (s, 1H), 10.18 (s, 1H).

### Example 258—Synthesis of N-(5-Methoxy-1-Methyl-1H-Indazol-7-yl)-1-(4-(Trifluoromethyl)Pyridin-2-yl)-1H-Pyrazole-4-Sulfonamide (I-220)

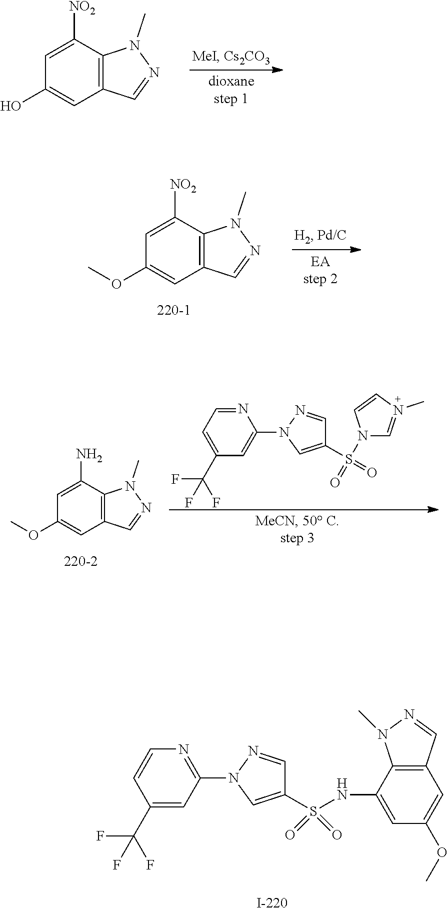

Step 1: Synthesis of 220-1. A solution of 1-methyl-7-nitro-1H-indazol-5-ol (120 mg, 0.621 mmol, 1 eq.) in dioxane (2 mL) was treated with Cs₂CO₃ (607.24 mg, 1.863 mmol, 3 eq.) for 2 min at 0° C. under a nitrogen atmosphere followed by the addition of Mel (132.27 mg, 0.931 mmol, 1.5 eq.) dropwise at 0° C. The resulting mixture was stirred for an additional 2 h at room temperature. The resulting mixture was filtered and the filter cake was washed with EtOAc (2×10 mL). The filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 220-1 (100 mg, 77.69%) as a white solid.

Step 2: Synthesis of 220-2. To a stirred solution of 220-1 (100 mg, 0.518 mmol, 1 eq.) in EA (2 mL) was added Pd/C (55.09 mg, 0.518 mmol, 1 eq.) in portions at room temperature under a nitrogen atmosphere. The resulting mixture was stirred for an additional 2 h at room temperature under a hydrogen atmosphere. The resulting mixture was filtered and the filter cake was washed with EtOAc (2×5 mL). The filtrate was concentrated under reduced pressure. The residue was purified by reversed-phase flash chromatography with the following conditions: column: C18 silica gel; mobile phase: MeCN in water (0.1% FA), 30% to 70% gradient in 20 min; detector: UV 254 nm. This resulted in 220-2 (80 mg, 78.48%) as a brown solid.

Step 3: Synthesis of N-(5-methoxy-1-methyl-1H-indazol-7-yl)-1-(4-(trifluoromethyl)pyridin-2-yl)-1H-pyrazole-4-sulfonamide (I-220). A solution of 220-2 (80 mg, 0.451 mmol, 1 eq.) in pyridine (5 mg) was treated with 3-methyl-1-((1-(4-(trifluoromethyl)pyridin-2-yl)-1H-pyrazol-4-yl)sulfonyl)-1H-imidazol-3-ium (168.84 mg, 0.541 mmol, 1.2 eq.) for 3 min at room temperature under a nitrogen atmosphere followed by the addition of DMAP (11.03 mg, 0.090 mmol, 0.2 eq.) in portions at room temperature. The resulting mixture was stirred for an additional 2 h at room temperature. The resulting mixture was concentrated under reduced pressure. The residue was purified by reversed-phase flash chromatography with the following conditions: column: C18 silica gel; mobile phase: MeCN in water (0.1% FA), 0% to 70% gradient in 20 min; detector: UV 254 nm. This resulted in I-220 (5.0 mg, 2.42%) as a white solid. (ES, m/z): [M+H]⁺=453; ¹H NMR: (400 MHz, DMSO) 3.66 (s, 3H), 4.22 (s, 3H), 6.38 (d, J=2.4 Hz, 1H), 7.15 (s, 1H), 7.90 (d, J=5.2 Hz, 1H), 7.95 (s, 1H), 8.14 (s, 1H), 8.21 (s, 1H), 8.83 (d, J=5.2 Hz, 1H), 8.92 (s, 1H), 10.13 (s, 1H).

### Example 259—Synthesis of N-(5-Fluoro-1-Methyl-1H-Indazol-7-yl)-1-(4-(Trifluoromethyl)Pyridin-2-yl)-1H-Pyrazole-4-Sulfonamide (I-221)

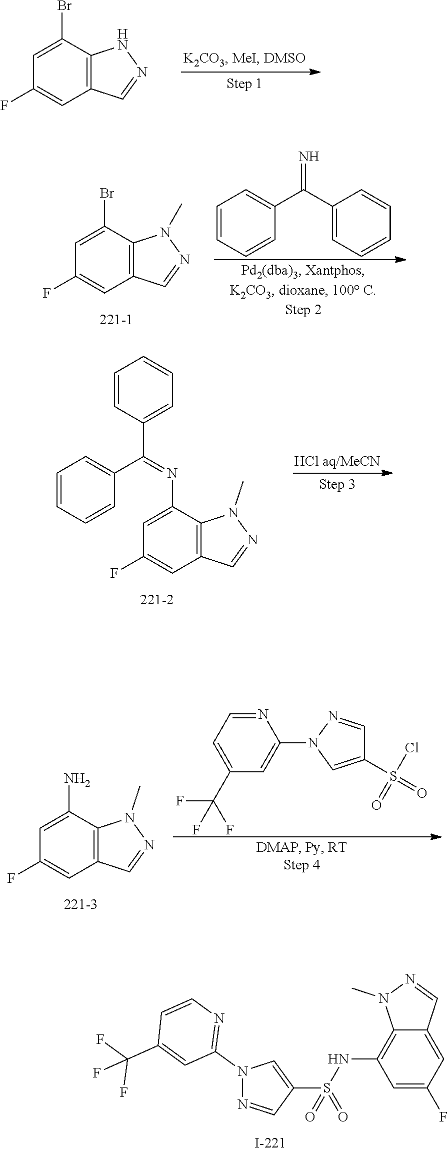

Step 1: Synthesis of 221-1. To a stirred solution of 7-bromo-5-fluoro-1H-indazole (1 g, 4.651 mmol, 1 eq.) and K₂CO₃ (1.93 g, 13.953 mmol, 3 eq.) in DMSO (20 mL) was added Mel (0.58 mL, 9.317 mmol, 2.00 eq.) dropwise at room temperature under a nitrogen atmosphere. The resulting mixture was stirred for 1 h at room temperature under a nitrogen atmosphere. The resulting mixture was filtered and the filter cake was washed with MeCN (3×10 mL). The filtrate was concentrated under reduced pressure. The residue was purified by reversed-phase flash chromatography with the following conditions: column: C18 silica gel; mobile phase: MeCN in water (10 mmol/L NH₄CO₃), 60% to 70% gradient in 40 min; detector: UV 215 nm. This resulted in 221-1 (500 mg, 46.94%) as a white solid.

Step 2: Synthesis of 221-2. To a stirred solution of 221-1 (500 mg, 2.183 mmol, 1 eq.), Pd₂(dba)₃ (400 mg, 0.437 mmol, 0.20 eq.), XantPhos (316 mg, 0.546 mmol, 0.25 eq.) and K₂CO₃ (905 mg, 6.548 mmol, 3.00 eq.) in dioxane (25 mL) was added diphenylmethanimine (550 uL, 3.277 mmol, 1.50 eq.) dropwise at room temperature under a nitrogen atmosphere. The resulting mixture was stirred for 15 h at 100° C. under a nitrogen atmosphere. The resulting mixture was diluted with water (2.5 mL). The resulting mixture was extracted with EtOAc (4×5 mL). The combined organic layers were washed with brine (10 mL) and dried over anhydrous Na₂SO₄. After filtration, the filtrate was concentrated and purified by reversed-phase flash chromatography with the following conditions: column: C18 silica gel; mobile phase: MeCN in water (10 mmol/L NH₄CO₃), 40% to 50% gradient in 10 min; detector: UV 210 nm. This resulted in 221-2 (450 mg, 62.59%) as a yellow solid.

Step 3: Synthesis of 221-3. To a stirred solution of 221-2 (450 mg, 1.366 mmol, 1 eq.) in MeCN (15 mL) was added 1 N HCl (60 mL) dropwise at room temperature under a nitrogen atmosphere. The resulting mixture was stirred for 1 h at room temperature under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄CO₃), 39% to 45% gradient in 15 min; detector: UV 220 nm. This resulted in 221-3 (190 mg, 84.20%) as a light brown solid.

Step 4: Synthesis of N-(5-fluoro-1-methyl-1H-indazol-7-yl)-1-(4-(trifluoromethyl)pyridin-2-yl)-1H-pyrazole-4-sulfonamide (I-221). To a stirred solution of 221-3 (100 mg, 0.605 mmol, 1 eq.) and pyridine (250 uL, 0.983 mmol, 5 eq.) in DCM (5 mL) was added 1-[4-(trifluoromethyl)pyridin-2-yl]pyrazole-4-sulfonyl chloride (227 mg, 0.728 mmol, 1.20 eq.) in portions at room temperature under a nitrogen atmosphere. The resulting mixture was stirred for 1 h at room temperature under a nitrogen atmosphere. The resulting mixture was concentrated under reduced pressure. The crude product (220 mg) was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 17% B to 44% B in 7 min; wavelength: 220 nm; Rt(min): 7.43) to afford I-221 (56.7 mg, 21.07%) as a white solid. (ES, m/z): [M+H]⁺ 441; ¹H NMR: (400 MHz, DMSO-d₆) δ 4.26 (s, 3H), 6.67 (dd, J=9.6, 2.4 Hz, 1H), 7.51 (d, J=8.0 Hz, 1H), 7.89 (d, J=5.2 Hz, 1H), 8.07 (s, 1H), 8.14 (s, 1H), 8.20 (s, 1H), 8.83 (d, J=5.2 Hz, 1H), 8.94 (s, 1H), 10.30 (s, 1H).

### Example 260—Synthesis of N-(5-Chloro-1-Methyl-1H-Indazol-7-yl)-1-(4-(Trifluoromethyl)Pyridin-2-yl)-1H-Pyrazole-4-Sulfonamide (I-222)

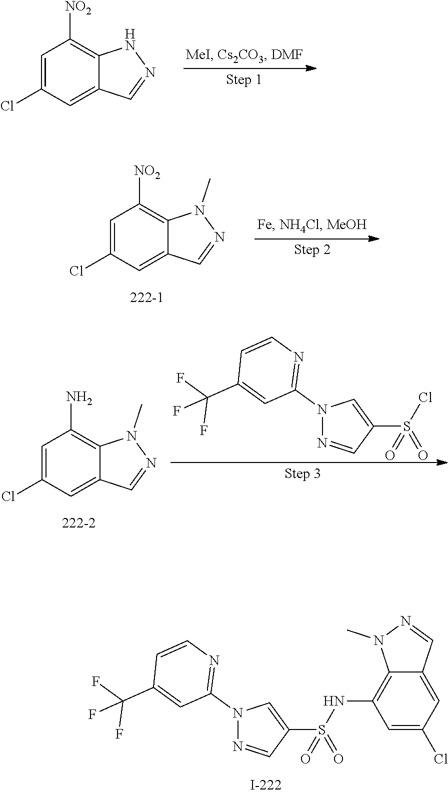

Step 1: Synthesis of 222-1. To a stirred mixture of 5-chloro-7-nitro-1H-indazole (2 g, 10.122 mmol, 1 eq.) and Cs₂CO₃ (8.25 g, 25.305 mmol, 2.5 eq.) in DMF (25 mL) was added CH₃I (0.95 mL, 15.183 mmol, 1.5 eq.) dropwise at room temperature under a nitrogen atmosphere. The resulting mixture was stirred for 2 h at room temperature under a nitrogen atmosphere. The resulting mixture was extracted with EtOAc (3×100 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in 222-1 (1.15 g, 53.69%) as a yellow solid.

Step 2: Synthesis of 222-2. To a stirred solution of 222-1 (500 mg, 2.363 mmol, 1 eq.) in MeOH (30 mL) were added Fe (659.76 mg, 11.815 mmol, 5 eq.) and NH₄Cl (631.94 mg, 11.815 mmol, 5 eq.) in portions at room temperature under a nitrogen atmosphere. The resulting mixture was stirred for 16 h at 80° C. under a nitrogen atmosphere. The resulting mixture was filtered and the filter cake was washed with MeOH (2×60 mL). The filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 222-2 (280 mg, 65.28%) as a black solid.

Step 3: Synthesis of N-(5-chloro-1-methyl-1H-indazol-7-yl)-1-(4-(trifluoromethyl) pyridin-2-yl)-1H-pyrazole-4-sulfonamide (I-222). To a stirred solution of 222-2 (150 mg, 0.826 mmol, 1 eq.) and 1-[4-(trifluoromethyl) pyridin-2-yl] pyrazole-4-sulfonyl chloride (308.88 mg, 0.991 mmol, 1.2 eq.) in pyridine (5 mL) was added DMAP (20.18 mg, 0.165 mmol, 0.2 eq.) at room temperature under a nitrogen atmosphere. The resulting mixture was stirred for 2 h at 50° C. under a nitrogen atmosphere. The resulting mixture was extracted with EtOAc (2×50 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 60% to 70% gradient in 10 min; detector: UV 254 nm. The resulting mixture was concentrated under reduced pressure. This resulted in I-222 (53.6 mg, 14.09%) as an off-white solid. (ES, m/z): [M+H]⁺ 457 1H NMR (400 MHz, DMSO-d₆) δ 4.25 (s, 3H), 6.71 (d, J=1.2 Hz, 1H), 7.84 (d, J=1.2 Hz, 1H), 7.90 (d, J=5.2 Hz, 1H), 8.09 (s, 1H), 8.15 (s, 1H), 8.21 (s, 1H), 8.83 (d, J=5.2 Hz, 1H), 8.92 (s, 1H), 10.28 (s, 1H).

### Example 261—Synthesis of 1-(4-(Tert-Butyl)Pyridin-2-yl)-N-(1-Methyl-1H-Indazol-7-yl)-1H-Pyrazole-4-Sulfonamide (I-223)

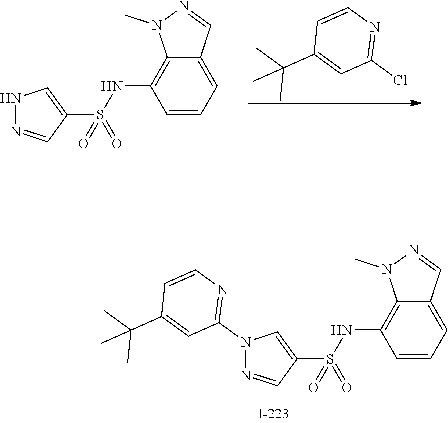

To a stirred solution of N-(1-methylindazol-7-yl)-1H-pyrazole-4-sulfonamide (100 mg, 0.361 mmol, 1 eq.), Cs₂CO₃ (352.49 mg, 1.083 mmol, 3 eq.) and 4-tert-butyl-2-chloropyridine (91.77 mg, 0.541 mmol, 1.5 eq.) in DMF (3 mL) were added N1, N2-dimethylcyclohexane-1,2-diamine (51.30 mg, 0.361 mmol, 1 eq.) and CuI (34.34 mg, 0.180 mmol, 0.5 eq.) dropwise at room temperature under a nitrogen atmosphere. The resulting mixture was stirred overnight at 140° C. The resulting mixture was quenched with water (30 mL) and extracted with CH₂Cl₂ (3×10 mL). The combined organic layers were dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 20% to 60% gradient in 20 min; detector: UV 254 nm. The crude product (60 mg) was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 31% B to 56% B in 7 min; wavelength: 220 nm; Rt(min): 8.43, to afford I-223 (20 mg, 13.48%) as a white solid. (ES, m/z): [M+H]⁺=411; ¹H NMR: ¹H NMR (400 MHz, DMSO-d₆) δ 1.34 (s, 9H), 4.29 (s, 3H), 6.71 (dd, J=7.2, 0.8 Hz, 1H), 6.97 (t, J=7.6 Hz, 1H), 7.52 (dd, J=5.2, 1.6 Hz, 1H), 7.69 (d, J=8.0 Hz, 1H), 7.95 (d, J=1.2 Hz, 1H), 8.01 (s, 1H), 8.08 (s, 1H), 8.43 (d, J=5.2 Hz, 1H), 8.79 (d, J=0.8 Hz, 1H), 10.02 (s, 1H).

### Example 262—Synthesis of 1-(4-(1-Hydroxy-3-Methylcyclobutyl) Pyridin-2-yl)-N-(1-Methyl-1H-Indazol-7-yl)-1H-Pyrazole-4-Sulfonamide (I-224)

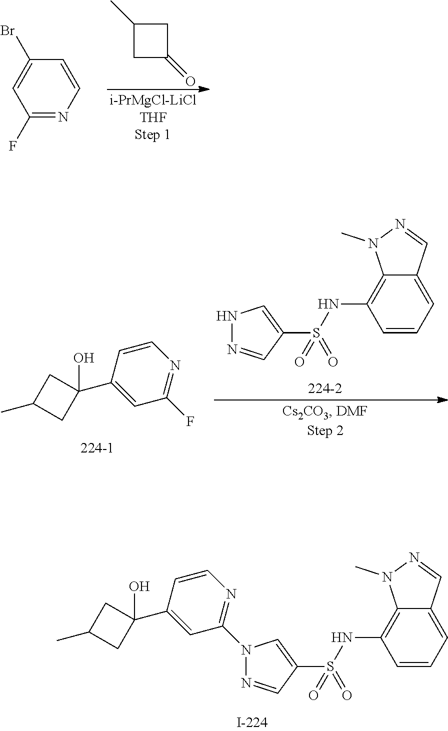

Step 1: Synthesis of 224-1. A solution of 4-bromo-2-fluoropyridine (700 mg, 3.978 mmol, 1 eq.) in THE (40 mL) was treated with isopropylmagnesium chloride (613.58 mg, 5.967 mmol, 1.5 eq.) for 30 min at −78° C. under a nitrogen atmosphere followed by the addition of 3-methylcyclobutan-1-one (401.50 mg, 4.774 mmol, 1.20 eq.) dropwise at −60° C. Then the mixture was stirred from −60° C. to room temperature overnight. After the reaction was completed, the mixture was quenched with saturated NH₄Cl (40 mL), extracted with EA (3×40 mL), washed with the brine (1×40 mL) and dried over anhydrous Na₂SO₄ to give the crude product. The residue was purified by Prep-TLC (PE/EA 4:1) to afford 224-1 (154 mg, 21.37%) as a yellow oil.

Step 2: Synthesis of 1-(4-(1-hydroxy-3-methylcyclobutyl)pyridin-2-yl)-N-(1-methyl-1H-indazol-7-yl)-1H-pyrazole-4-sulfonamide (I-224). A solution of 224-1 (108.6 mg, 0.599 mmol, 1 eq.) and 224-2 (166.19 mg, 0.599 mmol, 1 eq.) in DMF (6 mL) was stirred for 2d at 80° C. After the reaction was complete, the mixture was purified by reversed-phase flash chromatography directly with the following conditions: column: C18 silica gel; mobile phase: ACN in water (10 mmol/L NH₄CO₃), 0% to 100% gradient in 40 min; detector: UV 220 nm to give I-224 (19.1 mg, 7.26%) pink solid as product. (ES, m/z): [M+H]⁺ 439; ¹H NMR (400 MHz, DMSO-d) δ 1.18 (d, J=6.4 Hz, 3H), 2.02-2.07 (m, 2H), 2.16-2.18 (m, 1H), 2.50-2.61 (m, 2H), 4.29 (s, 3H), 6.00 (s, 1H), 6.71 (d, J=7.2 Hz, 1H), 6.97 (t, J=8.0 Hz, 1H), 7.56 (dd, J=5.2, 1.2 Hz, 1H), 7.67 (d, J=7.6 Hz, 1H), 8.01 (s, 1H), 8.07 (s, 1H), 8.08 (s, 1H), 8.46 (d, J=5.2 Hz, 1H), 8.79 (s, 1H), 10.04 (s, 1H).

### Example 263—Synthesis of 1-(2-Chloropyridin-4-yl)-N-(1-Methyl-1H-Indazol-7-yl)-1H-Pyrazole-4-Sulfonamide (I-225)

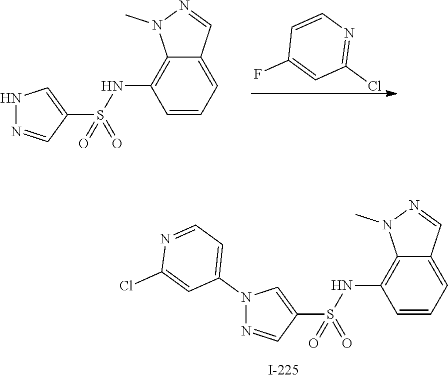

A solution of N-(1-methylindazol-7-yl)-1H-pyrazole-4-sulfonamide (100 mg, 0.361 mmol, 1 eq.), 2-chloro-4-fluoropyridine (71.15 mg, 0.541 mmol, 1.5 eq.), CuI (34.34 mg, 0.180 mmol, 0.5 eq.), N1,N2-dimethylcyclohexane-1,2-diamine (51.30 mg, 0.361 mmol, 1 eq.) and Na₂CO₃ (114.66 mg, 1.083 mmol, 3 eq.) in DMF (1.5 mL) was stirred for 16 h at 110° C. under a nitrogen atmosphere. The reaction was quenched with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(2-chloropyridin-4-yl)-N-(1-methylindazol-7-yl)pyrazole-4-sulfonamide (2.3 mg, 1.64%) as a white solid. (ES, m/z): [M+H]⁺ 389; ¹H NMR: (400 MHz, DMSO-d₆) δ 4.29 (s, 3H), 6.74 (d, J=7.6 Hz, 1H), 6.97-7.02 (m, 1H), 7.71 (d, J=8.0 Hz, 1H), 8.00-8.01 (m, 1H), 8.09-8.14 (m, 3H), 8.53 (dd, J=5.6, 2.0 Hz, 1H), 9.24 (s, 1H), 10.13 (s, 1H).

### Example 264—Synthesis of N-(1,3-Dimethyl-1H-Indazol-7-yl)-1-(4-(Trifluoromethyl)Pyridin-2-yl)-1H-Pyrazole-4-Sulfonamide (I-226)

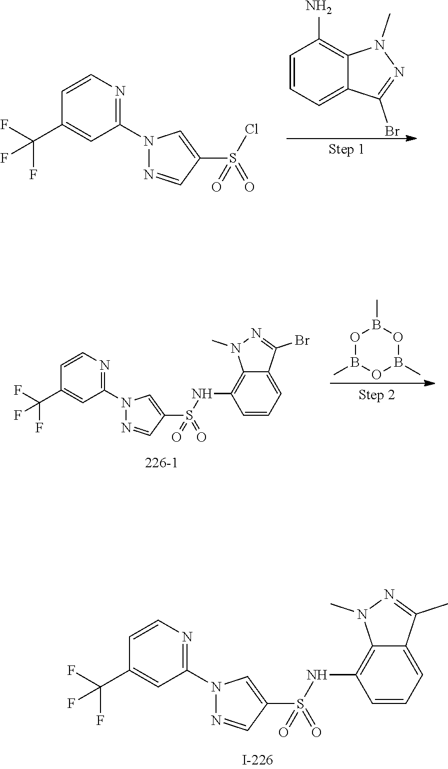

Step 1: Synthesis of 226-1. A solution of 1-(4-(trifluoromethyl)pyridin-2-yl)-1H-pyrazole-4-sulfonyl chloride (400 mg, 1.283 mmol, 1.00 eq.) and 3-bromo-1-methylindazol-7-amine (348.1 mg, 1.540 mmol, 1.20 eq.) in pyridine (4 mL) was stirred for 1 day at 80° C. under a nitrogen atmosphere. The mixture was allowed to cool down to room temperature. The reaction was quenched with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 15 min; detector: UV 254 nm. This resulted in N-(3-bromo-1-methyl-1H-indazol-7-yl)-1-(4-(trifluoromethyl)pyridin-2-yl)-1H-pyrazole-4-sulfonamide (287 mg, 44.43%) as a grey solid.

Step 2: Synthesis of N-(1,3-dimethyl-1H-indazol-7-yl)-1-(4-(trifluoromethyl)pyridin-2-yl)-1H-pyrazole-4-sulfonamide (I-226). A solution of N-(3-bromo-1-methyl-1H-indazol-7-yl)-1-(4-(trifluoromethyl)pyridin-2-yl)-1H-pyrazole-4-sulfonamide (100 mg, 0.199 mmol, 1.00 eq.), trimethyl-1,3,5,2,4,6-trioxatriborinane (75.1 mg, 0.597 mmol, 3.00 eq.), K₂CO₃ (82.7 mg, 0.597 mmol, 3.00 eq.), XPhos (19.0 mg, 0.040 mmol, 0.20 eq.) and Pd₂(dba)₃ (18.2 mg, 0.020 mmol, 0.10 eq.) in dioxane (16 mL)/H₂O (4 mL) was stirred for 2 h at 80° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 24% B to 51% B in 7 min; wavelength: 254 nm/220 nm; Rt(min): 8.02 to afford N-(1,3-dimethyl-1H-indazol-7-yl)-1-(4-(trifluoromethyl) pyridin-2-yl)-1H-pyrazole-4-sulfonamide (4.8 mg, 5.15%) as an off-white solid. (ES, m/z): [M+H]⁺ 437; ¹H NMR: (DMSO-d₆, 400 MHz) δ 2.45 (s, 3H), 4.20 (s, 3H), 6.72 (d, J=7.2 Hz, 1H), 6.91 (t, J=7.6 Hz, 1H), 7.58 (d, J=7.6 Hz, 1H), 7.88 (d, J=4.8 Hz, 1H), 8.07 (s, 1H), 8.19 (s, 1H), 8.81-8.83 (m, 2H), 10.04 (s, 1H).

### Example 265—Synthesis of N-(4-Chloro-1-Methyl-1H-Indazol-7-yl)-1-(4-(Trifluoromethyl)Pyridin-2-yl)-1H-Pyrazole-4-Sulfonamide (I-227)

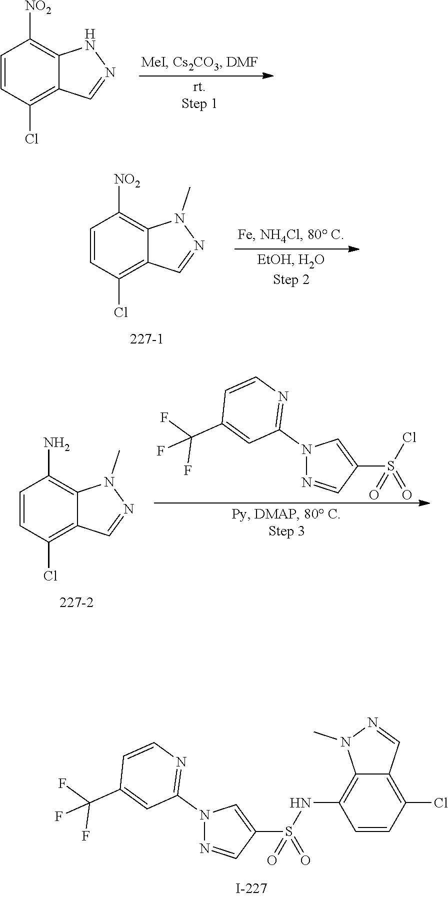

Step 1: Synthesis of 227-1. To a stirred solution of 4-chloro-7-nitro-1H-indazole (1 g, 5.061 mmol, 1 eq.) and Cs₂CO₃ (4.95 g, 15.183 mmol, 3 eq.) in DMF (10 mL) was added Mel (2.16 g, 15.183 mmol, 3 eq.) dropwise at 0° C. under a nitrogen atmosphere. The resulting mixture was stirred for 2 h at room temperature under a nitrogen atmosphere. The resulting mixture was extracted with EtOAc (2×200 mL). The combined organic layers were washed with brine (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 227-1 (650 mg, 60.69%) as a yellow solid.

Step 2: Synthesis of 227-2. A mixture of 227-1 (640 mg, 3.024 mmol, 1 eq.), Fe (506.70 mg, 9.072 mmol, 3 eq.) and NH₄Cl (242.67 mg, 4.536 mmol, 1.5 eq.) in EtOH (10 mL) was stirred for 4 h at 80° C. under a nitrogen atmosphere. The resulting mixture was filtered and the filter cake was washed with MeOH (2×20 mL). The filtrate was concentrated under reduced pressure. The residue was purified by reversed-phase flash chromatography with the following conditions: column: C18 silica gel; mobile phase: MeCN in water (0.1% FA), 20% to 30% gradient in 10 min; detector: UV 254 nm. This resulted in 227-2 (500 mg, 91.03%) as an off-white solid.

Step 3: Synthesis of N-(4-chloro-1-methyl-1H-indazol-7-yl)-1-(4-(trifluoromethyl) pyridin-2-yl)-1H-pyrazole-4-sulfonamide (I-227). A solution of 227-2 (116.55 mg, 0.642 mmol, 2 eq.), 1-[4-(trifluoromethyl) pyridin-2-yl]pyrazole-4-sulfonyl chloride (100 mg, 0.321 mmol, 1.00 eq.) and DMAP (3.92 mg, 0.032 mmol, 0.1 eq.) in pyridine (5 mL) was stirred overnight at 50° C. under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in I-227 (32.9 mg, 22.29%) as a pink solid. (ES, m/z): [M+H]⁺ 457; ¹H NMR: (400 MHz, DMSO-d₆) δ 4.30 (s, 3H), 6.72 (d, J=8.0 Hz, 1H), 7.08 (d, J=8.0 Hz, 1H), 7.89 (d, J=5.2 Hz, 1H), 8.12 (s, 1H), 8.14 (s, 1H), 8.20 (s, 1H), 8.83 (d, J=5.2 Hz, 1H), 8.90 (s, 1H), 10.15 (s, 1H).

### Example 266—Synthesis of N-(1,4-Dimethyl-1H-Indazol-7-yl)-1-(4-(Trifluoromethyl)Pyridin-2-yl)-1H-Pyrazole-4-Sulfonamide (I-228)

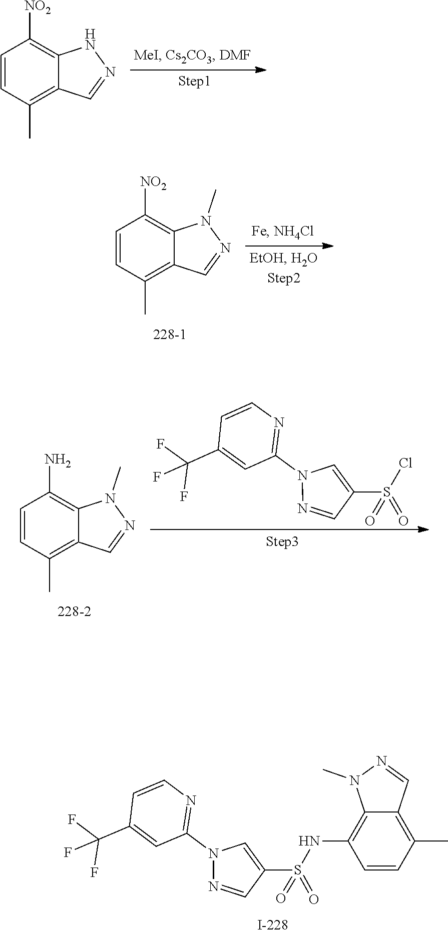

Step 1: Synthesis of 228-1. To a stirred solution of 4-methyl-7-nitro-1H-indazole (2 g, 11.289 mmol, 1 eq.) and Mel (2.40 g, 16.933 mmol, 1.50 eq.) in DMF (30 mL) was added Cs₂CO₃ (7.36 g, 22.578 mmol, 2.00 eq.) in portions at room temperature under a nitrogen atmosphere. The resulting mixture was extracted with EtOAc (2×200 mL). The combined organic layers were washed with brine (4×200 mL) and dried over anhydrous Na₂SO₄. After filtration, the filtrate was concentrated under reduced pressure. The residue was purified by silica gel column chromatography and eluted with PE/EA (1:1) to afford 228-1 (1.1 g, 50.96%) as a yellow solid.

Step 2: Synthesis of 228-2. To a stirred solution of 228-1 (1 g, 5.230 mmol, 1 eq.) and Fe (1.46 g, 26.150 mmol, 5 eq.) in EtOH (10 mL) were added NH₄Cl (1.40 g, 26.150 mmol, 5 eq.) and AcOH (0.63 g, 10.460 mmol, 2 eq.) in portions at room temperature under a nitrogen atmosphere. The resulting mixture was stirred for 2 h at 80° C. under a nitrogen atmosphere. The resulting mixture was filtered and the filter cake was washed with EtOAc (2×100 mL). The filtrate was concentrated under reduced pressure to afford 228-2 (800 mg, 75.90%) as a yellow solid.

Step 3: Synthesis of N-(1,4-dimethyl-1H-indazol-7-yl)-1-(4-(trifluoromethyl)pyridin-2-yl)-1H-pyrazole-4-sulfonamide (I-228). To a stirred solution of 228-2 (100 mg, 0.620 mmol, 1 eq.) and 1-[4-(trifluoromethyl)pyridin-2-yl]pyrazole-4-sulfonyl chloride (231.99 mg, 0.744 mmol, 1.2 eq.) in pyridine (5 mL) was added DMAP (15.16 mg, 0.124 mmol, 0.2 eq.) at room temperature. The resulting mixture was stirred for 2 h at 50° C. under a nitrogen atmosphere. The mixture was allowed to cool down to room temperature. The resulting mixture was extracted with EtOAc (2×50 L). The combined organic layers were washed with brine (2 mL) and dried over anhydrous Na₂SO₄. After filtration, the filtrate was concentrated under reduced pressure. The crude product (100 mg) was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 29% B to 56% B in 7 min; wavelength: 254 nm/220 nm; Rt(min): 8.5) to afford I-228 (46.1 mg, 16.94%) as a yellow solid. (ES, m/z): [M+H]⁺=437; ¹H NMR: ¹H NMR (400 MHz, DMSO-d₆) δ 2.50 (s, 3H), 4.27 (s, 3H), 6.60 (d, J=7.2 Hz, 1H), 6.74 (d, J=7.6 Hz, 1H), 7.89 (d, J=5.2 Hz, 1H), 8.09 (s, 1H), 8.12 (s, 1H), 8.20 (s, 1H), 8.82 (d, J=4.8 Hz, 1H), 8.87 (s, 1H), 9.97 (s, 1H).

### Example 267—Synthesis of N-(4-Fluoro-1-Methyl-1H-Indazol-7-yl)-1-(4-(Trifluoromethyl)Pyridin-2-yl)-1H-Pyrazole-4-Sulfonamide (I-229)

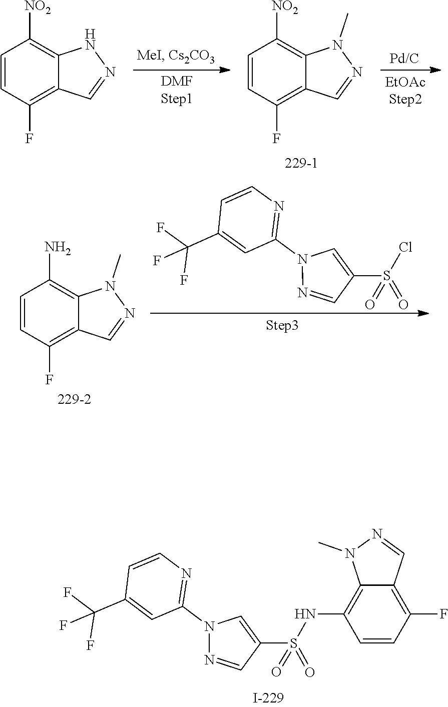

Step 1: Synthesis of 229-1. A solution of 4-fluoro-7-nitro-1H-indazole (500.00 mg, 2760.509 mmol, 1 eq.) in DMF (3 mL) was treated with Cs₂CO₃ (2698.28 mg, 8281.527 mmol, 3 eq.) for 30 min at room temperature under a nitrogen atmosphere followed by the addition of methyl iodide (470.19 mg, 3312.611 mmol, 1.2 eq.) dropwise at room temperature. The resulting mixture was stirred for 4 h at room temperature. The reaction was quenched with water (20 mL). The resulting mixture was extracted with EtOAc (3×20 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (1:1) to afford 4-fluoro-1-methyl-7-nitro-1H-indazole (200.00 mg, 0.04%) as a yellow oil.

Step 2: Synthesis of 229-2. To a solution of 4-fluoro-1-methyl-7-nitro-1H-indazole (100.00 mg, 0.512 mmol, 1 eq.) in 5 mL EtOAc was added Pd/C (10%, 5.45 mg) under a nitrogen atmosphere. The mixture was hydrogenated at room temperature for 2 h under a hydrogen atmosphere. The resulting mixture was filtered through a celite pad and concentrated under reduced pressure to afford 4-fluoro-1-methyl-1H-indazol-7-amine (50.00 mg, 56.77%) as an off-white solid.

Step 3: Synthesis of N-(4-fluoro-1-methyl-1H-indazol-7-yl)-1-(4-(trifluoromethyl) pyridin-2-yl)-1H-pyrazole-4-sulfonamide (I-229). A mixture of 4-fluoro-1-methyl-1H-indazol-7-amine (60.00 mg, 0.363 mmol, 1 eq.) and 1-(4-(trifluoromethyl)pyridin-2-yl)-1H-pyrazole-4-sulfonyl chloride (135.86 mg, 0.436 mmol, 1.2 eq.) in pyridine (2 mL) was stirred for 30 min at 80° C. The reaction was quenched with water (20 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 25% B to 53% B in 7 min; wavelength: 254 nm/220 nm; Rt(min): 8.38, to afford N-(4-fluoro-1-methyl-1H-indazol-7-yl)-1-(4-(trifluoromethyl)pyridin-2-yl)-1H-pyrazole-4-sulfonamide (30.00 mg, 18.72%) as an off-white solid. (ES, m/z): [M+H]⁺=441; ¹H NMR: (400 MHz, DMSO-d₆) δ 4.30 (s, 3H), 6.69-6.78 (m, 2H), 7.89 (d, J=5.2 Hz, 1H), 8.11 (s, 1H), 8.20 (s, 2H), 8.83 (d, J=5.2 Hz, 1H), 8.89 (s, 1H), 10.06 (s, 1H).

### Example 268—Synthesis of N-(3-Methoxy-1-Methyl-1H-Indazol-7-yl)-1-(4-(Trifluoromethyl)Pyridin-2-yl)-1H-Pyrazole-4-Sulfonamide (I-230)

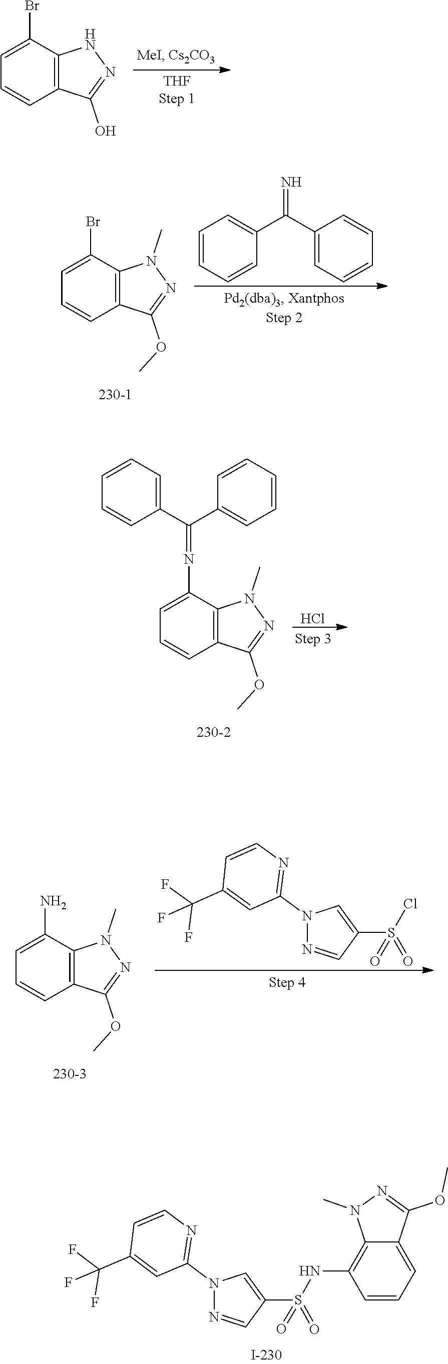

Step 1: Synthesis of 230-1. A mixture of 7-bromo-1H-indazol-3-ol (1 g, 4.694 mmol, 1.00 eq.) and Cs₂CO₃ (4.59 g, 14.082 mmol, 3.00 eq.) in THF (40 mL) was stirred for 30 min at room temperature. To the above mixture was added CH₃I (2.00 g, 14.082 mmol, 3.00 eq.) in portions at room temperature. The resulting mixture was stirred for an additional 2 h at room temperature. The resulting mixture was quenched with water (40 mL) and extracted with EtOAc (2×40 mL). The combined organic layers were washed with brine (100 mL) and dried over anhydrous Na₂SO₄. After filtration, the filtrate was concentrated under reduced pressure. The residue was purified by silica gel column chromatography and eluted with PE/EA (1:1) to afford 7-bromo-3-methoxy-1-methyl-1H-indazole (140 mg, 12.37%) as a yellow solid.

Step 2: Synthesis of 230-2. Into a 40 mL vial were added 7-bromo-3-methoxy-1-methyl-1H-indazole (140 mg, 0.581 mmol, 1.00 eq.), diphenylmethanimine (315.7 mg, 1.743 mmol, 3.00 eq.), Cs₂CO₃ (567.6 mg, 1.743 mmol, 3.00 eq.) and dioxane (30 mL) at room temperature. To the above mixture was added XantPhos (168.0 mg, 0.290 mmol, 0.50 eq.) and Pd₂(dba)₃ (60.1 mg, 0.058 mmol, 0.10 eq.) at room temperature. The resulting mixture was stirred overnight at 90° C. under a nitrogen atmosphere. The reaction was quenched with water (80 mL) at room temperature and extracted with EtOAc (2×200 mL). The combined organic layers were washed with brine (100 mL) and dried over anhydrous Na₂SO₄. After filtration, the filtrate was concentrated under reduced pressure to afford N-(3-methoxy-1-methyl-1H-indazol-7-yl)-1,1-diphenylmethanimine (190 mg, crude) as a yellow solid.

Step 3: Synthesis of 230-3. A solution of N-(3-methoxy-1-methyl-1H-indazol-7-yl)-1,1-diphenylmethanimine (190 mg, crude) in H₂O (2 mL) and HCl/dioxane (6M, 0.5 mL) was stirred for 1 h at room temperature. The resulting mixture was extracted with EtOAc (2×20 mL). The combined organic layers were washed with brine (20 mL) and dried over anhydrous Na₂SO₄. After filtration, the filtrate was concentrated under reduced pressure. The residue was purified by Prep-TLC (PE/EA 1:1) to afford crude 3-methoxy-1-methyl-1H-indazol-7-amine (90 mg, crude) as a pink solid.

Step 4: Synthesis of N-(3-methoxy-1-methyl-1H-indazol-7-yl)-1-(4-(trifluoromethyl) pyridin-2-yl)-1H-pyrazole-4-sulfonamide (I-230). A mixture of 3-methoxy-1-methyl-1H-indazol-7-amine (50 mg, 0.282 mmol, 1.00 eq.) and 1-(4-(trifluoromethyl)pyridin-2-yl)-1H-pyrazole-4-sulfonyl chloride (263.81 mg, 0.846 mmol, 3.00 eq.) in pyridine (2 mL) was stirred overnight at 80° C. under a nitrogen atmosphere. The reaction was quenched with water (10 mL) at room temperature and extracted with EtOAc (3×20 mL). The combined organic layers were washed with brine (50 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; flow rate: 60 mL/min; gradient: 15% B to 45% B in 8 min, 45% B; wavelength: 220 nm; Rt(min): 7.58 to afford N-(3-methoxy-1-methyl-1H-indazol-7-yl)-1-(4-(trifluoromethyl)pyridin-2-yl)-1H-pyrazole-4-sulfonamide (22.2 mg, 17.39%) as an off-white solid. (ES, m/z): [M+H]+ 453; ¹H NMR: (400 MHz, DMSO-d₆) δ 3.99 (s, 3H), 4.09 (s, 3H), 6.74 (d, J=6.8 Hz, 1H), 6.87 (t, J=7.6 Hz, 1H), 7.52 (d, J=7.6 Hz, 1H), 7.88 (d, J=5.2 Hz, 1H), 8.10 (s, 1H), 8.20 (s, 1H), 8.81-8.86 (m, 2H), 10.04 (s, 1H).

### Example 269—Synthesis of 1-(4-(1-Methoxy-3-Methylcyclobutyl) Pyridin-2-yl)-N-(1-Methyl-1H-Indazol-7-yl)-1H-Pyrazole-4-Sulfonamide (I-231)

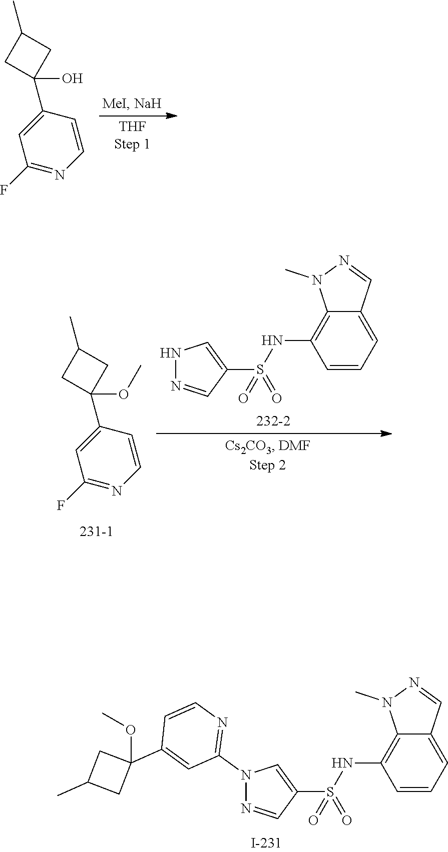

Step 1: Synthesis of 231-1. A solution of 1-(2-fluoropyridin-4-yl)-3-methylcyclobutan-1-ol (80 mg, 0.441 mmol, 1 eq.) in THE (2 ml) was treated with NaH (52.97 mg, 2.207 mmol, 5.00 eq.) for 30 min at 0° C. under a nitrogen atmosphere followed by the addition of Mel (156.66 mg, 1.103 mmol, 2.5 eq.) dropwise. The resulting mixture was stirred overnight at room temperature. The reaction was quenched with water at 0° C. The aqueous layer was extracted with EtOAc (3×50 mL). After filtration, the filtrate was concentrated under reduced pressure. The residue was purified by Prep-TLC (PE/EA 5:1) to afford as 231-1 (50 mg, 58.01%) a brown solid.

Step 2: Synthesis of 1-(4-(1-methoxy-3-methylcyclobutyl)pyridin-2-yl)-N-(1-methyl-1H-indazol-7-yl)-1H-pyrazole-4-sulfonamide (I-231). A mixture of 231-1 (50 mg, 0.256 mmol, 1 eq.), Cs₂CO₃ (250.33 mg, 0.768 mmol, 3 eq.) and 232-2 (71.02 mg, 0.256 mmol, 1 eq.) in DMF (2 ml) was stirred overnight at 80° C. under a nitrogen atmosphere. The reaction was quenched with water at room temperature. The aqueous layer was extracted with EtOAc (3×80 mL). After filtration, the filtrate was concentrated under reduced pressure. The residue was purified by reversed-phase flash chromatography with the following conditions: column: C18 silica gel; mobile phase: MeCN in water (0.1% TFA), 40% to 70% gradient in 20 min; detector: UV 254 nm to afford I-231 (8.1 mg, 6.93%) as a white solid. (ES, m/z): [M+H]⁺ 453; ¹H NMR:(400 MHz, DMSO-d₆) δ 1.16 (d, J=6.4 Hz, 3H), 1.96-2.10 (m, 3H), 2.50-2.58 (m, 2H), 2.93 (s, 3H), 4.29 (s, 3H), 6.73 (d, J=7.2 Hz, 1H), 6.98 (t, J=7.6 Hz, 1H), 7.54 (dd, J=5.2, 1.6 Hz, 1H), 7.71 (d, J=7.6 Hz, 1H), 7.97 (s, 1H), 8.03 (s, 1H), 8.09 (s, 1H), 8.54 (d, J=4.8 Hz, 1H), 8.82 (s, 1H), 10.03 (s, 1H).

### Example 270—Synthesis of 1-(4-(1-Fluoro-3-Methylcyclobutyl) Pyridin-2-yl)-N-(1-Methyl-1H-Indazol-7-yl)-1H-Pyrazole-4-Sulfonamide (I-232)

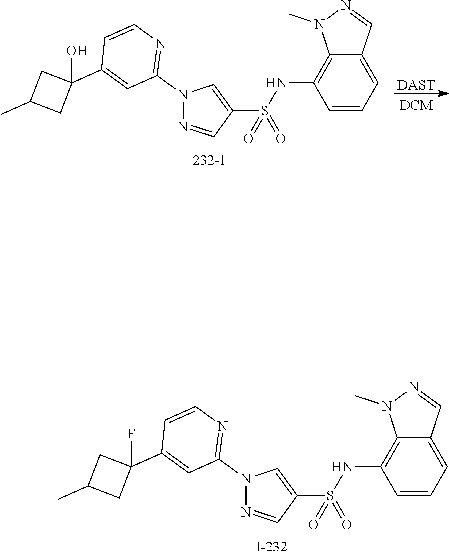

A solution of 232-1 (91.5 mg, 0.209 mmol, 1 eq.) and DAST (134.54 mg, 0.836 mmol, 4 eq.) in DCM (5 mL) was stirred overnight from 0° C. to room temperature. After the reaction was completed, the solution was quenched with a saturated NaHCO₃ solution (25 mL) and extracted with DCM (4×50 mL). Then the organic phases were combined, washed with the brine (50 mL) and concentrated under vacuum to give the mixture. The mixture was purified by reversed-phase flash chromatography with the following conditions: column: C18 silica gel; mobile phase: ACN in water (10 mmol/L NH₄CO₃), 0% to 100% gradient in 30 min; detector: UV 254 nm, to give the crude product. The residue was purified by prep-HPLC with the following conditions: column: XBridge Prep OBD C₁₈ column, 30*150 mm, 5 μm; mobile phase A: water (10 mmol/L NH₄CO₃)+0.05% NH₃·H₂O, mobile phase B: ACN; flow rate: 60 mL/min; gradient: 34% B to 60% B in 7 min; wavelength: 254 nm/220 nm; Rt(min): 7.85) to afford 1-232 (2.6 mg, 2.80%) as a pink solid. (ES, m/z): [M+H]⁺ 441; ¹H NMR: ¹H NMR (400 MHz, DMSO-d) δ1.19-2.07 (m, 3H), 2.24-2.33 (m, 2H), 2.65-2.78 (m, 3H), 4.29 (s, 3H), 6.71 (d, J=7.2 Hz, 1H), 6.96 (t, J=7.6 Hz, 1H), 7.56 (d, J=5.6 Hz, 1H), 7.67 (d, J=7.6 Hz, 1H), 7.96 (d, J=7.6 Hz, 1H), 8.04 (s, 1H), 8.08 (s, 1H), 8.56 (d, J=5.2 Hz, 1H), 8.81 (d, J=2.0 Hz, 1H), 10.04 (s, 1H).

### Example 271—Synthesis of 1-(2-Chloropyridin-4-yl)-N-(3-Fluoro-2-Methyl-2H-Indazol-7-yl)-1H-Pyrazole-4-Sulfonamide (I-233)

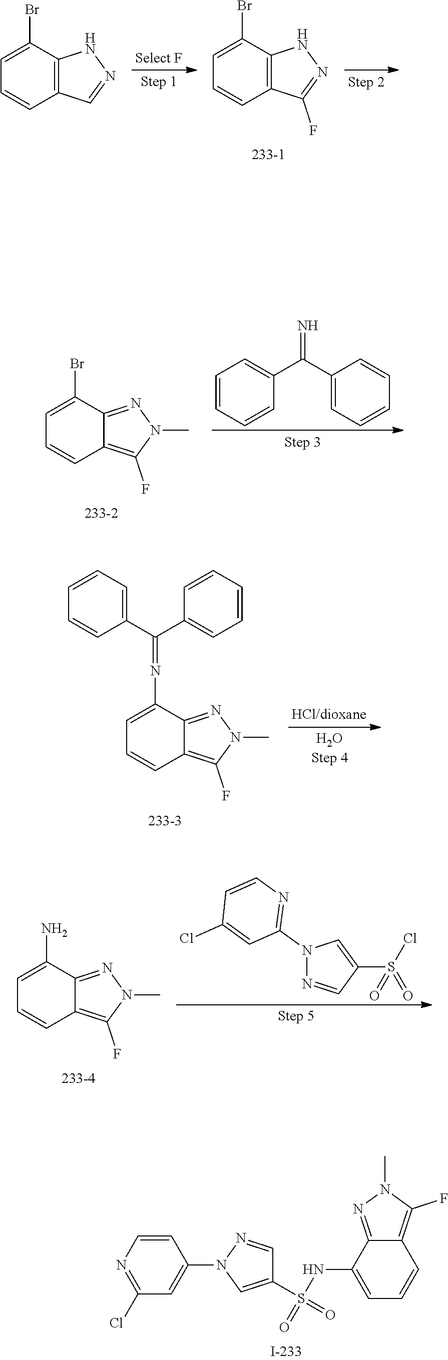

Step 1: Synthesis of 233-1. A solution of 7-bromo-1H-indazole (4 g, 20.301 mmol, 1.00 eq.) and Selectfluor (10.79 g, 30.451 mmol, 1.50 eq.) in MeCN (50 mL) was stirred overnight at 70° C. under a nitrogen atmosphere. The resulting mixture was concentrated under reduced pressure. The residue was purified by silica gel column chromatography and eluted with PE/EA (12:1) to afford 7-bromo-3-fluoro-1H-indazole (1.2 g, 27.49%) as a light yellow solid.

Step 2: Synthesis of 233-2. To a stirred solution of 7-bromo-3-fluoro-1H-indazole (500 mg, 2.325 mmol, 1.00 eq.) in THF (5 mL) were added t-BuONa (670.42 mg, 6.975 mmol, 3.00 eq.) at 0° C. under a nitrogen atmosphere. The resulting mixture was stirred for 30 min at room temperature under a nitrogen atmosphere. To the above mixture was added CH₃I (495.0 mg, 3.488 mmol, 1.50 eq.) dropwise at room temperature. The resulting mixture was stirred for 1 h at room temperature under a nitrogen atmosphere. The reaction was quenched by the addition of sat. NH₄Cl (aq.) (10 mL) at room temperature. The resulting mixture was extracted with EtOAc (3×10 mL). The combined organic layers were washed with brine (30 mL) and dried over anhydrous Na₂SO₄. After filtration, the filtrate was concentrated under reduced pressure. The residue was purified by silica gel column chromatography and eluted with PE/EA (12:1) to afford 7-bromo-3-fluoro-1H-indazole (250 mg, 32.86%) as a light yellow solid.

Step 3: Synthesis of 233-3. A solution of 7-bromo-3-fluoro-1H-indazole (130.0 mg, 0.568 mmol, 1.00 eq.), Pd₂(dba)₃ (103.9 mg, 0.114 mmol, 0.20 eq.), XantPhos (82.1 mg, 0.142 mmol, 0.25 eq.), Cs₂CO₃ (554.7 mg, 1.704 mmol, 3.00 eq.) and diphenylmethanimine (123.4 mg, 0.682 mmol, 1.20 eq.) in dioxane (2 mL) was stirred for 2 h at 80° C. under a nitrogen atmosphere. The resulting mixture was diluted with water (10 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The residue was purified by silica gel column chromatography and eluted with PE/EA (5:1) to afford N-(3-fluoro-2-methyl-2H-indazol-7-yl)-1,1-diphenylmethanimine (120.0 mg, crude) as a light yellow solid.

Step 4: Synthesis of 233-4. A solution of N-(3-fluoro-2-methyl-2H-indazol-7-yl)-1,1-diphenylmethanimine (130.0 mg, 0.395 mmol, 1.00 eq.) in HCl/dioxane (6M, 4 mL) and H₂O (4 mL) was stirred for 30 min at room temperature under a nitrogen atmosphere. The mixture was basified to pH 8 with saturated NaHCO₃ (aq.). The resulting mixture was extracted with EtOAc (3×10 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (5:1) to afford 3-fluoro-2-methyl-2H-indazol-7-amine (60.0 mg, crude) as a light yellow solid.

Step 5: Synthesis of 1-(2-chloropyridin-4-yl)-N-(3-fluoro-2-methyl-2H-indazol-7-yl)-1H-pyrazole-4-sulfonamide (I-233). A solution of 3-fluoro-2-methyl-2H-indazol-7-amine (50.0 mg, 0.303 mmol, 1.00 eq.) and 1-(4-chloropyridin-2-yl)-1H-pyrazole-4-sulfonyl chloride (92.6 mg, 0.333 mmol, 1.10 eq.) in pyridine (5 mL) was stirred for 2 h at 80° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 30% B to 56% B in 7 min; wavelength: 220 nm; Rt(min): 6.83, to afford 1-(2-chloropyridin-4-yl)-N-(3-fluoro-2-methyl-2H-indazol-7-yl)-1H-pyrazole-4-sulfonamide (22 mg, 19.55%) as an off-white solid. (ES, m/z): [M+H]+ 407; ¹H NMR: (400 MHz, DMSO-d₆) δ 4.00 (s, 3H), 6.96 (t, J=8.0 Hz, 1H), 7.18 (d, J=7.2 Hz, 1H), 7.34 (d, J=8.4 Hz, 1H), 7.61 (dd, J=5.2, 1.6 Hz, 1H), 7.95 (d, J=1.6 Hz, 1H), 8.19 (s, 1H), 8.51 (d, J=5.2 Hz, 1H), 9.09 (s, 1H), 10.40 (s, 1H).

### Example 272—Synthesis of N-(3-Fluoro-1-Methyl-1H-Indazol-7-yl)-1-(4-(Trifluoromethyl)Pyridin-2-yl)-1H-Pyrazole-4-Sulfonamide (I-234)

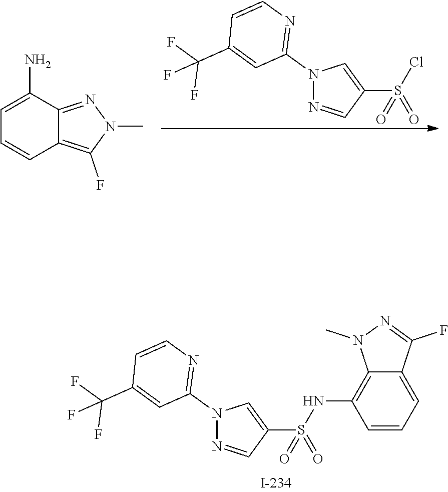 [SMILES: Cn1nc2c(N)cccc2c1F ; O=S(=O)(Cl)c1cnn(-c2cc(C(F)(F)F)ccn2)c1 ; Cn1nc(F)c2cccc(NS(=O)(=O)c3cnn(-c4cc(C(F)(F)F)ccn4)c3)c21]

A solution of 3-fluoro-2-methyl-2H-indazol-7-amine (45.0 mg, 0.272 mmol, 1.00 eq.) and 1-(4-(trifluoromethyl)pyridin-2-yl)-1H-pyrazole-4-sulfonyl chloride (93.4 mg, 0.299 mmol, 1.10 eq.) in pyridine (2 mL) was stirred for 2 h at 80° C. under a nitrogen atmosphere. The mixture was allowed to cool down to room temperature. The resulting mixture was diluted with water (10 mL) and extracted with EtOAc (3×10 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 32% B to 60% B in 7 min; wavelength: 220 nm; Rt(min): 8.32 to afford N-(3-fluoro-1-methyl-1H-indazol-7-yl)-1-(4-(trifluoromethyl)pyridin-2-yl)-1H-pyrazole-4-sulfonamide (16.3 mg, 13.54%) as an off-white solid. (ES, m/z): [M+H]+ 441; ¹H NMR: (400 MHz, DMSO-d₆) δ 4.01 (s, 3H), 6.97 (t, J=7.9 Hz, 1H), 7.19 (d, J=7.2 Hz, 1H), 7.35 (d, J=8.4 Hz, 1H), 7.86 (d, J=4.8 Hz, 1H), 8.12 (s, 1H), 8.23 (s, 1H), 8.82 (d, J=4.8 Hz, 1H), 9.17 (s, 1H), 10.43 (s, 1H).

### Example 273—Synthesis of 1-(5-(2-Hydroxypropan-2-yl)-4-Methylpyridin-2-yl)-N-(1-Methyl-1H-Indazol-7-yl)-1H-Pyrazole-4-Sulfonamide (I-235)

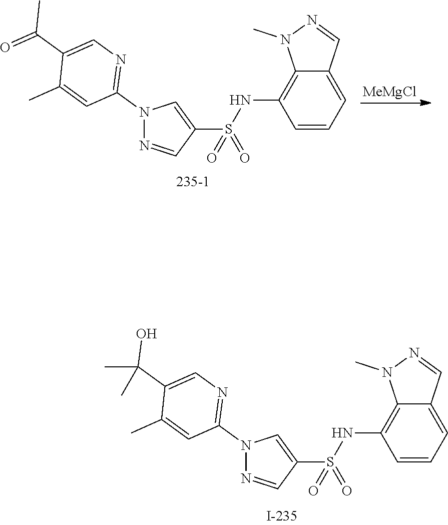 [SMILES: "MeMgCl" ; CC(=O)c1cnc(-n2cc(S(=O)(=O)Nc3cccc4cnn(C)c34)cn2)cc1C ; Cc1cc(-n2cc(S(=O)(=O)Nc3cccc4cnn(C)c34)cn2)ncc1C(C)(C)O]

To a stirred solution of 235-1 (25 mg, 0.061 mmol, 1 eq.) in THE (10 mg) was added MeMgCl (3M in THF, 0.041 mL, 0.122 mmol, 2 eq.) in portions at 0° C. under a nitrogen atmosphere. The resulting mixture was stirred for an additional 2 h at room temperature. The resulting mixture was quenched with water (1 mL) and extracted with CH₂Cl₂ (5×1 mL). The combined organic layers were washed with brine (1 mL) and dried over anhydrous Na₂SO₄. After filtration, the filtrate was concentrated under reduced pressure. The crude product (25 mg) was purified by prep-HPLC with the following conditions: column: YMC-Actus Triart C18 ExRS, 30*150 mm, 5 μm; mobile phase A: water (10 mmol/L NH₄CO₃+0.05% NH₃H₂O), mobile phase B: ACN; flow rate: 60 mL/min; gradient: 10% B to 35% B in 7 min; wavelength: 220 nm; Rt(min): 8.17;). This resulted in 1-235 (6.5 mg, 25.77%) as a white solid. (ES, m/z): [M+H]⁺=426; ¹H NMR (400 MHz, DMSO-d₆) δ 1.56 (s, 6H), 2.65 (s, 3H), 4.30 (s, 3H), 5.22 (s, 1H), 6.71 (d, J=7.2 Hz, 1H), 6.93 (t, J=7.6 Hz, 1H), 7.57 (s, 1H), 7.74 (s, 1H), 7.96 (s, 1H), 8.03 (s, 1H), 8.47 (s, 1H), 8.70 (s, 1H).

### Example 274—Synthesis of N-(1,5-Dimethyl-1H-Indazol-7-yl)-1-(4-(Trifluoromethyl)Pyridin-2-yl)-1H-Pyrazole-4-Sulfonamide (I-236)

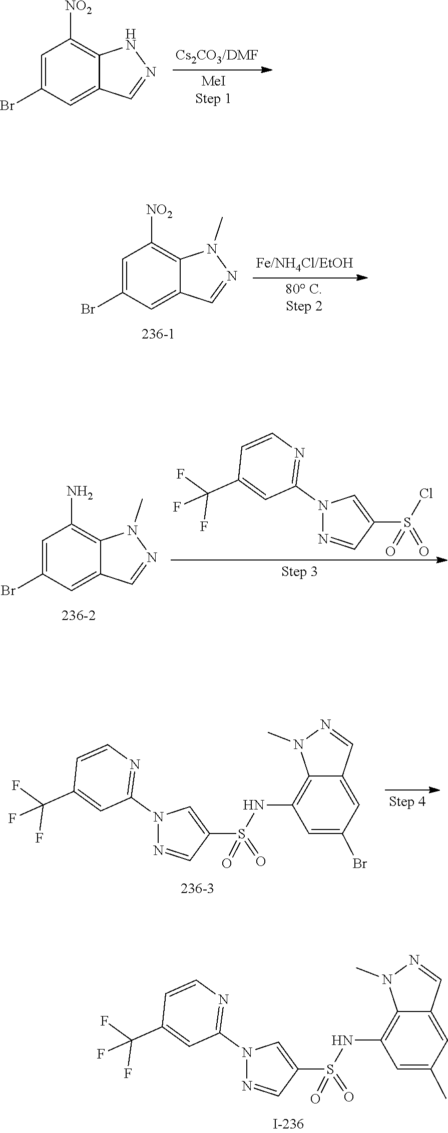

Step 1: Synthesis of 236-1. A solution of 5-bromo-7-nitro-1H-indazole (500 mg, 2.066 mmol, 1.00 eq.) and Cs₂CO₃ (2019.2 mg, 6.198 mmol, 3.00 eq.) in DMF (5 mL) was stirred for 0.5 h at 50° C. To the above mixture was added Mel (879.6 mg, 6.198 mmol, 3.00 eq.) dropwise at room temperature. The resulting mixture was stirred for an additional 2 h at 50° C. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 15 min; detector: UV 254 nm. This resulted in 5-bromo-1-methyl-7-nitro-1H-indazole (160 mg, 27.16%) as a yellow solid.

Step 2: Synthesis of 236-2. A solution of 5-bromo-1-methyl-7-nitro-1H-indazole (345 mg, 1.347 mmol, 1.00 eq.), Fe (376.2 mg, 6.735 mmol, 5.00 eq.) and NH₄Cl (360.3 mg, 6.735 mmol, 5.00 eq.) in EtOH (6 mL) was stirred for 12 h at 80° C. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. This resulted in 5-bromo-1-methyl-1H-indazol-7-amine (355 mg, 97.78%) as a black solid.

Step 3: Synthesis of 236-3. A solution of 5-bromo-1-methyl-1H-indazol-7-amine (130 mg, 0.575 mmol, 1.00 eq.) and 1-[4-(trifluoromethyl)pyridin-2-yl]pyrazole-4-sulfonyl chloride (179.2 mg, 0.575 mmol, 1.00 eq.) in pyridine (4 mL) was stirred for 2 h at 80° C. The mixture was allowed to cool down to room temperature. The resulting mixture was diluted with water (20 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 15 min; detector: UV 254 nm. This resulted in N-(5-bromo-1-methyl-1H-indazol-7-yl)-1-(4-(trifluoromethyl)pyridin-2-yl)-1H-pyrazole-4-sulfonamide (170 mg, 52.67%) as a grey solid.

Step 4: Synthesis of N-(1,5-dimethyl-1H-indazol-7-yl)-1-(4-(trifluoromethyl)pyridin-2-yl)-1H-pyrazole-4-sulfonamide (I-236). A solution of N-(5-bromo-1-methyl-1H-indazol-7-yl)-1-(4-(trifluoromethyl)pyridin-2-yl)-1H-pyrazole-4-sulfonamide (100 mg, 0.199 mmol, 1.00 eq.), trimethyl-1,3,5,2,4,6-trioxatriborinane (75.1 mg, 0.597 mmol, 3.00 eq.), Pd(dppf)Cl₂ (14.6 mg, 0.020 mmol, 0.10 eq.) and Cs₂CO₃ (194.9 mg, 0.597 mmol, 3.00 eq.) in dioxane (10 mL) was stirred for 2 h at 100° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃⁺0.05% NH₃·H₂O), mobile phase B: ACN; flow rate: 60 mL/min; gradient: 25% B to 52% B in 7 min; wavelength: 220 nm; Rt(min): 8.37 to afford (1,5-dimethyl-1H-indazol-7-yl)-1-(4-(trifluoromethyl)pyridin-2-yl)-1H-pyrazole-4-sulfonamide (5.1 mg, 5.82%) as an off-white solid. (ES, m/z): [M+H]⁺ 437; ¹H NMR: (DMSO-d₆, 400 MHz) δ 2.20 (s, 3H), 4.24 (s, 3H), 6.57 (s, 1H), 7.40 (s, 1H), 7.88 (d, J=4.4 Hz, 1H), 7.94 (s, 1H), 8.10 (s, 1H), 8.20 (s, 1H), 8.81-8.83 (m, 2H), 10.04 (s, 1H).

### Example 275—Synthesis of 3-Methyl-N-(1-Methyl-1H-Indazol-7-yl)-1-(4-(Trifluoromethyl)Pyridin-2-yl)-1H-Pyrazole-4-Sulfonamide (I-237)

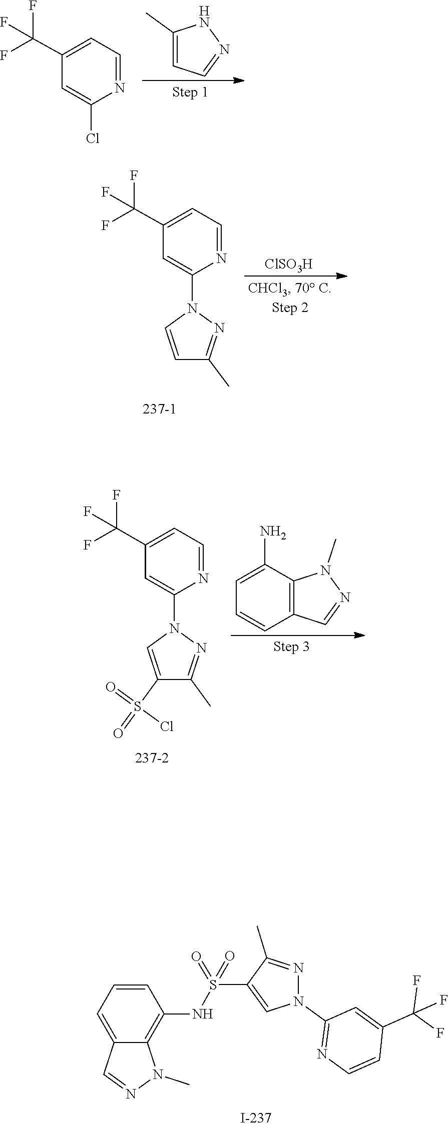

Step 1: Synthesis of 237-1. A solution of 2-chloro-4-(trifluoromethyl)pyridine (2 g, 11.017 mmol, 1 eq.) 3-methyl-2H-pyrazole (2.71 g, 33.051 mmol, 3 eq.) and t-BuOK (3.71 g, 33.051 mmol, 3 eq.) in ACN (15 mL) was stirred overnight at 80° C. The resulting mixture was diluted with water (50 mL) and extracted with EtOAc (3×30 mL). The combined organic layers were washed with brine (100 mL) and dried over anhydrous Na₂SO₄. After filtration, the filtrate was concentrated under reduced pressure. This resulted in 237-1 (1.19 g, 47.54%) as a yellow oil.

Step 2: Synthesis of 237-2. Into a 100 ml round bottom flask were added 237-1 (1 g, 4.402 mmol, 1 eq.) and chlorosulfonic acid (5 mL) at 0° C. Then the resulting mixture was stirred overnight at 80° C. The resulting mixture was quenched with dilute hydrochloric acid (7 g ice and 3 mL conc HCl). The resulting mixture was extracted with DCM (3×20 mL) and dried over anhydrous Na₂SO₄. The organic was concentrated under reduced pressure. This resulted in 237-2 (240 mg, 16.74%) as a brown solid.

Step 3. Synthesis of 3-methyl-N-(1-methyl-1H-indazol-7-yl)-1-(4-(trifluoromethyl) pyridin-2-yl)-1H-pyrazole-4-sulfonamide (I-237). A solution of 237-2 (448.10 mg, 1.376 mmol, 1.5 eq.) and 1-methylindazol-7-amine (135 mg, 0.917 mmol, 1.00 eq.) in pyridine (6 mL) was stirred for 3 hours at room temperature. The resulting mixture was concentrated under reduced pressure. The residue was dissolved in water (20 mL) and extracted with EtOAc (3×15 mL). The combined organic layers were washed with brine (30 mL) and dried over anhydrous Na₂SO₄. After filtration, the filtrate was concentrated under reduced pressure. The crude product (45 mg) was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 29% B to 59% B in 7 min; wavelength: 254 nm/220 nm; Rt(min): 7.8;) to afford I-237 (19.2 mg, 4.70%) as an off-white solid. (ES, m/z): [M+H]⁺ 436; ¹H NMR: (400 MHz, DMSO-d₆) δ 2.13 (s, 3H), 4.28 (s, 3H), 6.70 (dd, J=7.2, 1.2 Hz, 1H), 6.96 (t, J=7.6 Hz, 1H), 7.70 (d, J=8.0 Hz, 1H), 7.83 (dd, J=5.2, 0.8 Hz, 1H), 8.09 (s, 1H), 8.12 (s, 1H), 8.72 (s, 1H), 8.78 (d, J=5.2 Hz, 1H), 10.03 (s, 1H).

### Example 276—Synthesis of 1-(4-(1-Methoxycyclobutyl)Pyridin-2-yl)-N-(1-Methyl-1H-Indazol-7-yl)-1H-Pyrazole-4-Sulfonamide (I-93)

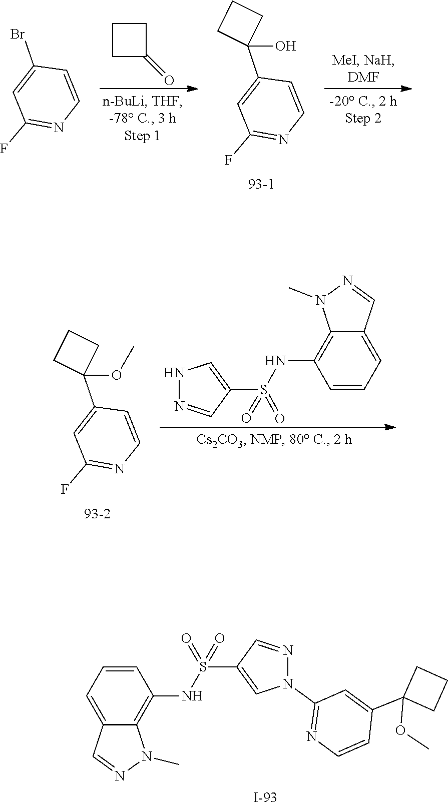

Step 1: Synthesis of 93-1. A solution of 4-bromo-2-fluoropyridine (500 mg, 2.841 mmol, 1 eq.) in THE (10 mL) was treated with butyllithium (3.4 mL, 8.500 mmol, 2.99 eq.) for 30 min at −78° C. under a nitrogen atmosphere followed by the addition of cyclobutanone (398.27 mg, 5.682 mmol, 2 eq.) dropwise at −78° C. The resulting mixture was stirred for 2.5 h at −78° C. to room temperature under a nitrogen atmosphere. The reaction was quenched with sat. NH₄Cl (aq.) at room temperature. The resulting mixture was extracted with CH₂Cl₂ (3×30 mL). The combined organic layers were washed with brine (50 mL) and dried over anhydrous Na₂SO₄. After filtration, the filtrate was concentrated under reduced pressure. The residue was purified by reverse phase flash with the following conditions: column: Xbridge Shield RP18 OBD column, 30*150 mm, 5 μm; mobile phase A: water (10 mmol/L NH₄CO₃+0.05% NH₃H₂O), mobile phase B: ACN; flow rate: 60 mL/min; gradient: 9% B to 33% B in 7 min; wavelength: 254 nm/220 nm; Rt(min): 7) to afford 93-1 (300 mg, 60.63%) as a light yellow oil.

Step 2: Synthesis of 93-2. A solution of 93-1 in DMF (8 mL) was added NaH (84 mg, 3.500 mmol, 1.95 eq.) at −20° C. under a nitrogen atmosphere. The resulting mixture was stirred for 15 min, followed by the addition of Mel (389 mg, 2.741 mmol, 1.53 eq.). The resulting mixture was stirred for an additional 2 h at −20° C. to room temperature under a nitrogen atmosphere. The reaction was quenched with sat. NH₄Cl (aq.) at room temperature. The resulting mixture was extracted with CH₂Cl₂ (3×50 mL). The combined organic layers were washed with brine (50 mL) and dried over anhydrous Na₂SO₄. After filtration, the filtrate was concentrated under reduced pressure. The residue was purified by silica gel column chromatography and eluted with PE/EA (1:1) to afford 93-2 (150 mg, 46.13%) as a brown oil.

Step 3: Synthesis of 1-(4-(1-methoxycyclobutyl)pyridine-2-yl)-N-(1-methyl-11H-indazol-7-yl)-1H-pyrazole-4-sulfonamide (I-93). A mixture of 93-2 (40 mg, 0.221 mmol, 1.5 eq.), N-(1-methylindazol-7-yl)-1H-pyrazole-4-sulfonamide (60 mg, 0.216 mmol, 0.98 eq.), and Cs₂CO₃ (144 mg, 0.442 mmol, 2.00 eq.) in NMP (4 mL) was stirred for 2 h at 80° C. under a nitrogen atmosphere. The resulting mixture was quenched with water (10 mL) and extracted with EtOAc (3×10 mL). The combined organic layers were washed with brine (20 mL) and dried over anhydrous Na₂SO₄. After filtration, the filtrate was concentrated under reduced pressure. The crude product (70 mg) was purified by prep-HPLC with the following conditions: column: Xbridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 15% B to 47% B in 8 min; wavelength: 220 nm; Rt(min): 6.35) to afford I-93 (30.7 mg, 47.53%) as an off-white solid. (ES, m/z): [M+H]+ 439; ¹H NMR (400 MHz, DMSO-d₆) δ 1.68-1.79 (m, 1H), 1.88-1.99 (m, 1H), 2.30-2.46 (m, 4H), 2.96 (s, 3H), 4.29 (s, 3H), 6.75 (d, J=7.2 Hz, 1H), 6.92-7.04 (m, 1H), 7.48-7.56 (m, 1H), 7.65 (d, J=8.0 Hz, 1H), 7.92-7.97 (m, 1H), 8.02 (s, 1H), 8.06 (s, 1H), 8.54 (d, J=5.2 Hz, 1H), 8.80 (s, 1H), 10.02 (s, 1H).

### Example 277—Synthesis of 1-(4-(1-Fluorocyclobutyl)Pyridin-2-yl)-N-(1-Methyl-1H-Indazol-7-yl)-1H-Pyrazole-4-Sulfonamide (I-94)

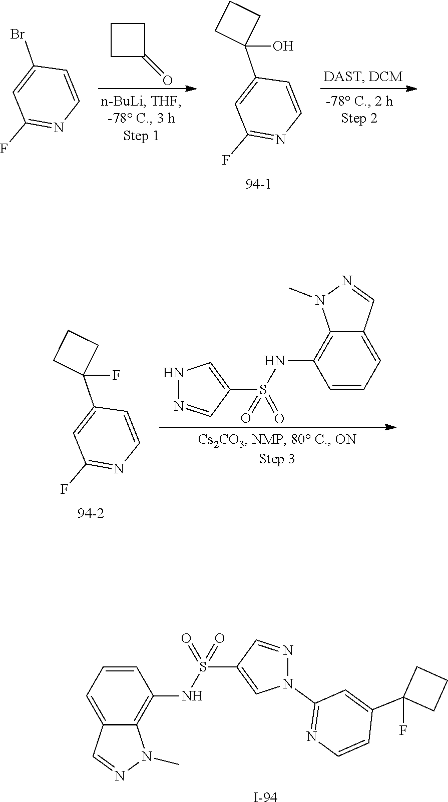

Step 1: Synthesis of 94-1. A solution of 4-bromo-2-fluoropyridine (500 mg, 2.841 mmol, 1 eq.) in THE (10 mL) was treated with butyllithium (3.4 mL, 8.500 mmol, 2.99 eq.) for 30 min at −78° C. under a nitrogen atmosphere followed by the addition of cyclobutanone (398.27 mg, 5.682 mmol, 2 eq.) dropwise at −78° C. The resulting mixture was stirred for 2.5 h at −78° C. to room temperature under a nitrogen atmosphere. The reaction was quenched with sat. NH₄Cl (aq.) at room temperature. The resulting mixture was extracted with CH₂Cl₂ (3×30 mL). The combined organic layers were washed with brine (50 mL) and dried over anhydrous Na₂SO₄. After filtration, the filtrate was concentrated under reduced pressure. The residue was purified by reverse phase flash with the following conditions: column: XBridge Shield RP18 OBD column, 30*150 mm, 5 μm; mobile phase A: water (10 mmol/L NH₄CO₃+0.05% NH₃H₂O), mobile phase B: ACN; flow rate: 60 mL/min; gradient: 9% B to 33% B in 7 min; wavelength: 254 nm/220 nm; Rt(min): 7;) to afford 94-1 (300 mg, 60.63%) as a light yellow oil.

Step 2: Synthesis of 94-2. A solution of 94-1 (200 mg, 1.196 mmol, 1 eq.) and DAST (88 mg, 2.373 mmol, 1.98 eq.) in DCM (8 mL) was stirred for 2 h at −78° C. under a nitrogen atmosphere. The resulting mixture was quenched with water (10 mL). The resulting mixture was extracted with CH₂Cl₂ (3×50 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 65% gradient in 10 min; detector: UV 254 nm. This resulted in 94-2 (82 mg, 40.52%) as a yellow oil.

Step 3: Synthesis of 1-(4-(1-fluorocyclobutyl)pyridin-2-yl)-N-(1-methyl-1H-indazol-7-yl)-1H-pyrazole-4-sulfonamide (I-94). A mixture of 94-2 (40 mg, 0.236 mmol, 1.5 eq.), N-(1-methylindazol-7-yl)-1H-pyrazole-4-sulfonamide (44 mg, 0.159 mmol, 0.67 eq.), Cs₂CO₃ (36 mg, 0.473 mmol, 2.00 eq.) in NMP (4 mL) was stirred overnight at 80° C. under a nitrogen atmosphere. The resulting mixture was extracted with EtOAc (3×10 mL). The combined organic layers were washed with brine (20 mL) and dried over anhydrous Na₂SO₄. After filtration, the filtrate was concentrated under reduced pressure. The crude product (60 mg) was purified by prep-HPLC with the following conditions: column: YMC-Actus Triant C18 ExRs column, 30*150 mm, 5 μm; mobile phase A: water (10 mmol/L NH₄CO₃+0.05% NH₃H₂O), mobile phase B: ACN; flow rate: 60 mL/min; gradient: 25% B to 55% B in 9 min; wavelength: 220 nm; Rt(min): 8.45) to afford I-94 (32.8 mg, 47.28%) as an off-white solid. (ES, m/z): [M+H]⁺ 427; ¹H NMR (400 MHz, DMSO-d₆) δ 1.79-1.94 (m, 1H), 2.03-2.16 (m, 1H), 2.55-2.91 (m, 4H), 4.30 (s, 3H), 6.75 (d, J=7.2 Hz, 1H), 6.93 (t, J=8.0 Hz, 1H), 7.58 (d, J=5.2 Hz, 2H), 7.71-8.20 (m, 3H), 8.56 (d, J=5.2 Hz, 1H), 8.78 (s, 1H), 10.02 (s, 1H).

### Example 278—Synthesis of N-(1-Methylimidazo[1,5-A]Pyridin-8-yl)-1-(4-(Trifluoromethyl)Pyridin-2-yl)-1H-Pyrazole-4-Sulfonamide (I-95)

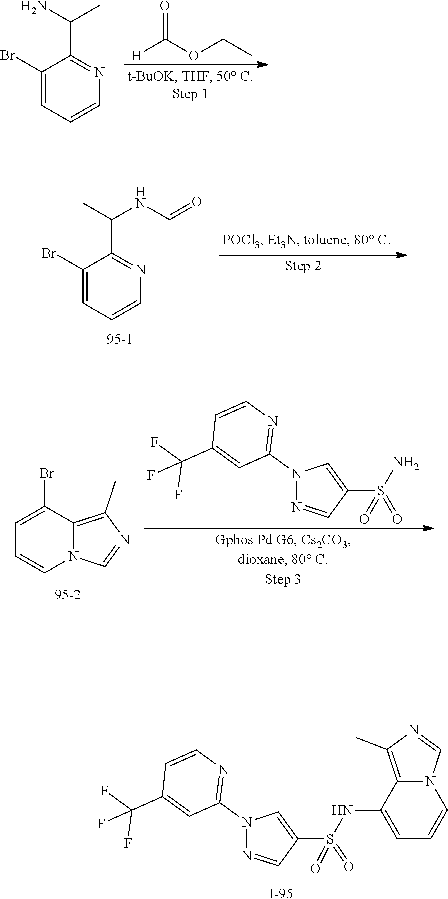

Step 1: Synthesis of 95-1. A solution of 1-(3-bromopyridin-2-yl)ethanamine (1 g, 4.973 mmol, 1 eq.) ethyl formate (0.44 g, 5.968 mmol, 1.2 eq.) and t-BuOK (1.12 g, 9.946 mmol, 2 eq.) in THF (10 mL) was stirred for 4 h at 50° C. under a nitrogen atmosphere. The resulting mixture was extracted with EtOAc (2×100 mL). The combined organic layers were washed with brine (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 95-1 (600 mg, 52.66%) as a white solid.

Step 2: Synthesis of 95-2. To a stirred solution of 95-1 (600 mg, 2.619 mmol, 1 eq.) and Et₃N (265.05 mg, 2.619 mmol, 1 eq.) in toluene (10 mL) was added POCl₃ (1204.73 mg, 7.857 mmol, 3 eq.) dropwise at 0° C. under a nitrogen atmosphere. The resulting mixture was stirred for 4 h at 80° C. under a nitrogen atmosphere. The resulting mixture was extracted with EtOAc (2×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 95-2 (400 mg, 72.36%) as an off-white solid.

Step 3: Synthesis of N-(1-methylimidazo[1,5-a]pyridin-8-yl)-1-(4-(trifluoromethyl)pyridin-2-yl)-1H-pyrazole-4-sulfonamide (I-95). A solution of 95-2 (100 mg, 0.321 mmol, 1.00 eq.), 1-(4-(trifluoromethyl)pyridin-2-yl)-1H-pyrazole-4-sulfonamide(166.86 mg, 0.385 mmol, 1.20 eq.), [3-(tert-butoxy)-6-methoxy-2′,6′-bis(propan-2-yl)-[1,1′-biphenyl]-2-yl]dicyclohexylphosphane (17.16 mg, 0.032 mmol, 0.1 eq.), Gphos pd G6 TES (29.47 mg, 0.032 mmol, 0.1 eq.) and Cs₂CO₃ (313.63 mg, 0.963 mmol, 3 eq.) in dioxane (20 mL) was stirred overnight at 100° C. under a nitrogen atmosphere. The resulting mixture was extracted with EtOAc (2×50 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in I-95 (60.1 mg, 44.03%) as a brown solid. (ES, m/z): [M+H]⁺ 423; ¹H NMR: (400 MHz, DMSO-d₆) δ 2.55 (s, 3H), 6.18 (d, J=6.8 Hz, 1H), 6.43 (t, J=7.2 Hz, 1H), 7.88 (d, J=4.8 Hz, 1H), 8.08 (d, J=6.8 Hz, 1H), 8.17 (s, 1H), 8.18 (s, 1H), 8.28 (s, 1H), 8.82 (d, J=4.8 Hz, 1H), 8.94 (s, 1H), 10.17 (s, 1H).

### Example 279—Synthesis of 1-(4-Cyclopropylpyridin-2-yl)-N-(6-(Methyoxy-D3)-1-Methyl-1H-Indazol-7-yl)-1H-Pyrazole-4-Sulfonamide (I-96)

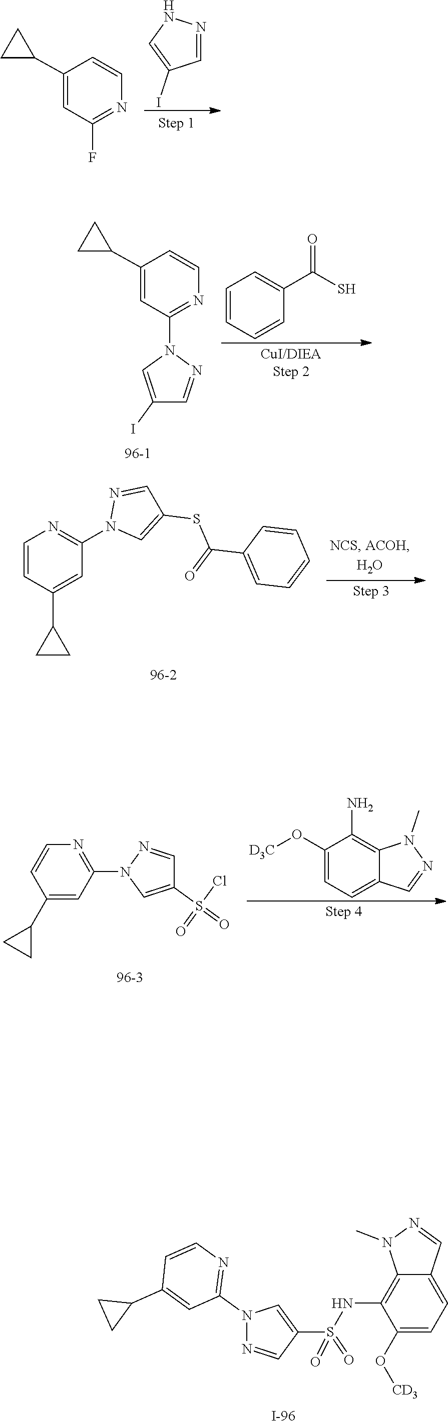

Step 1: Synthesis of 96-1. To a stirred mixture of 4-cyclopropyl-2-fluoropyridine (4.6 g, 33.538 mmol, 1 eq.) and 4-iodopyrazole (6.51 g, 33.538 mmol, 1 eq.) in DMF (50 mL) was added Cs₂CO₃ (32.78 g, 100.614 mmol, 3 eq.) in portions at room temperature under a nitrogen atmosphere. The resulting mixture was stirred overnight at 80° C. under a nitrogen atmosphere. The resulting mixture was quenched with water (100 mL) and extracted with EtOAc (3×100 mL). The combined organic layers were washed with brine (200 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 96-1 (4 g, 34.50%) as an off-white solid.

Step 2: Synthesis of 96-2. To a stirred mixture of 96-1 (4 g, 12.857 mmol, 1 eq.) and benzenecarbothioic acid (2.13 g, 15.428 mmol, 1.2 eq.) in toluene (20 mL) were added CuI (0.24 g, 1.286 mmol, 0.1 eq.) and DIEA (3.32 g, 25.714 mmol, 2 eq.) at room temperature under a nitrogen atmosphere. The resulting mixture was stirred overnight at 110° C. under a nitrogen atmosphere. The resulting mixture was quenched with water (50 mL) and extracted with EtOAc (5×50 mL). The combined organic layers were washed with brine (200 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 0% to 100% gradient in 20 min; detector: UV 254 nm. The resulting mixture was concentrated under vacuum. This resulted in 96-2 (4.3 g, 93.66%) as an off-white solid.

Step 3: Synthesis of 96-3. A mixture of 96-2 (2.1 g, 6.534 mmol, 1.00 eq.) and NCS (2.62 g, 19.602 mmol, 3 eq.) in AcOH (10 mL) and H₂O (1 mL) was stirred for 2 h at room temperature under a nitrogen atmosphere. The resulting mixture was diluted with water (50 mL) and extracted with EtOAc (3×50 mL). The combined organic layers were washed with brine (50 mL) and dried over anhydrous Na₂SO₄. After filtration, the filtrate was concentrated under reduced pressure. The residue was purified by silica gel column chromatography and eluted with PE/EA (12:1) to afford 96-3 as an off-white oil.

Step 4: Synthesis of 1-(4-cyclopropylpyridin-2-yl)-N-(6-(methoxy-d3)-1-methyl-1H-indazol-7-yl)-1H-pyrazole-4-sulfonamide (I-96). A solution of 96-3 (100 mg, 0.352 mmol, 1 eq.) in pyridine (1 mL) was treated with 6-(methoxy-d3)-1-methyl-1H-indazol-7-amine (63.52 mg, 0.352 mmol, 1 eq.) overnight at 80° C. under a nitrogen atmosphere. The resulting mixture was concentrated under vacuum. The resulting mixture was diluted with water (20 mL) and extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in I-96 (3.4 mg, 2.26%) as yellow solid. (ES, m/z): [M+H]⁺ 428; ¹H NMR: (400 MHz, DMSO-d₆) δ 0.91-0.93 (m, 2H), 1.13-1.17 (m, 2H), 2.10-2.16 (m, 1H), 4.24 (s, 3H), 6.86 (d, J=8.8 Hz, 1H), 7.13 (d, J=5.2 Hz, 1H), 7.65-7.70 (m, 2H), 7.95 (s, 1H), 7.97 (s, 1H), 8.31 (d, J=5.2 Hz, 1H), 8.67 (s, 1H), 9.71 (s, 1H).

### Example 280—Synthesis of 1-(4-Cyclopropylpyridin-2-yl)-N-(6-Methoxy-1-(Methyl-D3)-1H-Indazol-7-yl)-1H-Pyrazole-4-Sulfonamide (I-97)

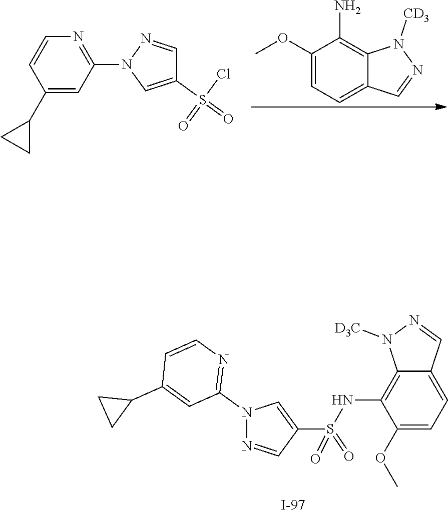

A solution of 1-(4-cyclopropylpyridin-2-yl)pyrazole-4-sulfonyl chloride (100 mg, 0.352 mmol, 1 eq.) in pyridine (1 mL) was treated with 6-methoxy-1-(methyl-d3)-1H-indazol-7-amine (63.52 mg, 0.352 mmol, 1 eq.) for 2 h at 80° C. under a nitrogen atmosphere. The resulting mixture was concentrated under vacuum. The resulting mixture was diluted with water (20 mL) and extracted with EtOAc (3×20 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 1-97 (11.6 mg, 7.53%) as a purple solid. (ES, m/z): [M+H]+ 428; ¹H NMR: (400 MHz, DMSO-d₆) δ 0.90-0.92 (m, 2H), 1.13-1.17 (m, 2H), 2.13-2.15 (m, 1H), 3.32 (s, 3H), 6.87 (d, J=9.2 Hz, 1H), 7.13 (dd, J=5.2, 1.6 Hz, 1H), 7.66-7.71 (m, 2H), 7.96 (s, 1H), 7.98 (s, 1H), 8.32 (d, J=5.2 Hz, 1H), 8.67 (s, 1H), 9.77 (s, 1H).

### Example 281—Synthesis of N-(1-(2-Methoxyethyl)-1H-Indazol-7-yl)-1-(4-(Trifluoromethyl)Pyridin-2-yl)-1H-Pyrazole-4-Sulfonamide (I-98)

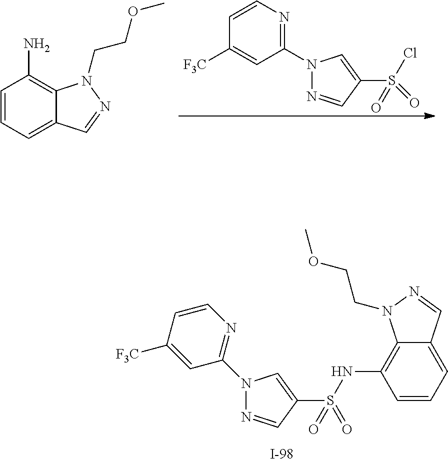

A mixture of 1-(2-methoxyethyl)-1H-indazol-7-amine (130.00 mg, 0.680 mmol, 1 eq.) and 1-(4-(trifluoromethyl)pyridin-2-yl)-1H-pyrazole-4-sulfonyl chloride (254.24 mg, 0.816 mmol, 1.2 eq.) in pyridine (2 mL) was stirred for 1 h at 80° C. The resulting mixture was quenched with water (10 mL) and extracted with EtOAc (3×15 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford N-(1-(2-methoxyethyl)-1H-indazol-7-yl)-1-(4-(trifluoromethyl)pyridin-2-yl)-1H-pyrazole-4-sulfonamide (53.50 mg, 16.82%) as an off-white solid. (ES, m/z): [M+H]⁺ 467; ¹H NMR: (400 MHz, DMSO-d₆) δ 3.14 (s, 3H), 3.74 (t, J=5.2 Hz, 2H), 4.53 (t, J=5.2 Hz, 2H), 6.97 (t, J=7.6 Hz, 1H), 7.16 (d, J=7.2 Hz, 1H), 7.47 (d, J=8.4 Hz, 1H), 7.83 (d, J=4.8 Hz, 1H), 8.10 (s, 1H), 8.19 (s, 1H), 8.33 (s, 1H), 8.80 (d, J=5.2 Hz, 1H), 9.09 (s, 1H), 10.27 (s, 1H).

### Example 282—Synthesis of 1-(4-(3-Fluoro-1-Methoxycyclobutyl) Pyridin-2-yl)-N-(1-Methyl-1H-Indazol-7-yl)-1H-Pyrazole-4-Sulfonamide (I-99)

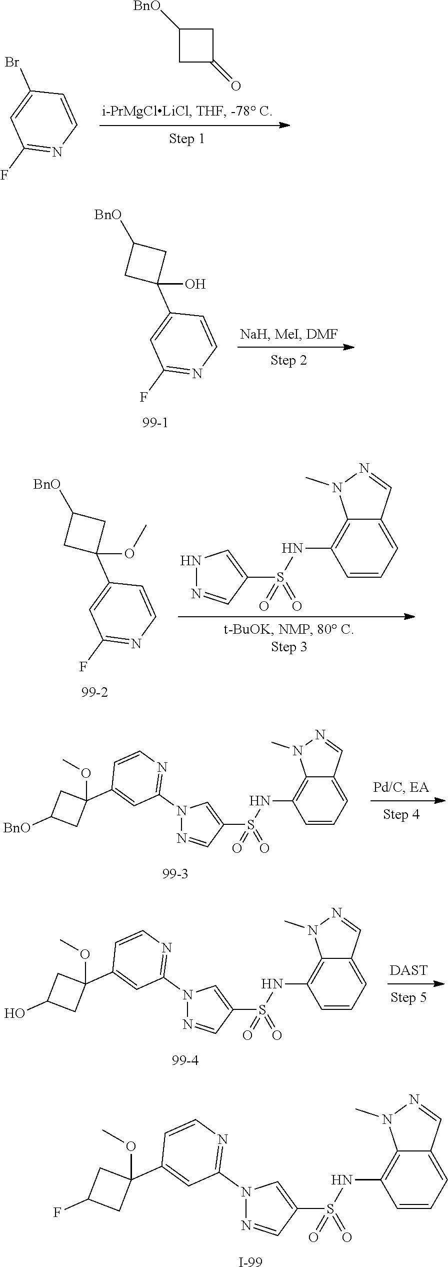

Step 1: Synthesis of 99-1. A solution of 4-bromo-2-fluoropyridine (5.7 g, 32.389 mmol, 1 eq.) in THE (60 mL) was treated with isopropylmagnesium chloride-lithium chloride complex (74.74 mL, 97.167 mmol, 3 eq.) for 1 h at −78° C. under a nitrogen atmosphere followed by the addition of 3-(benzyloxy)cyclobutan-1-one (17.12 g, 97.167 mmol, 3 eq.) in THE (180 mL) in portions at −60° C. The resulting mixture was stirred for 2 h at −60° C. under a nitrogen atmosphere. The reaction was quenched with 2 M HCl at 0° C. The aqueous layer was extracted with EtOAc (3×250 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm, to afford 99-1 (3 g, 33.89%) as a yellow solid.

Step 2: Synthesis of 99-2. A solution of 99-1 (1.1 g, 4.025 mmol, 1 eq.) in DMF (11 mL) was treated with NaH (144.88 mg, 6.038 mmol, 1.5 eq.) for 0.5 h at room temperature under a nitrogen atmosphere followed by the addition of Mel (685.53 mg, 4.830 mmol, 1.2 eq.) in portions at 0° C. The resulting mixture was stirred for 2 h at room temperature under a nitrogen atmosphere. The reaction was quenched with 2 M HCl at 0° C. The aqueous layer was extracted with EtOAc (3×100 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 60% to 70% gradient in 10 min; detector: UV 254 nm, to afford 99-2 (700 mg, 60.53%) as a yellow oil.

Step 3: Synthesis of 99-3. To a stirred solution of 99-2 (440 mg, 1.531 mmol, 1 eq.) and N-(1-methylindazol-7-yl)-1H-pyrazole-4-sulfonamide (594.49 m g, 2.143 mmol, 1.4 eq.) in NMP (9 mL) was added t-BuOK (687.34 mg, 6.124 mmol, 4 eq.) in portions at room temperature under a nitrogen atmosphere. The resulting mixture was stirred for 2 h at 80° C. under a nitrogen atmosphere. The reaction was quenched with 2 M HCl at 0° C. The aqueous layer was extracted with EtOAc (3×100 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 40% gradient in 10 min; detector: UV 254 nm, to afford 99-3 (700 mg, 83.93%) as a yellow oil.

Step 4: Synthesis of 99-4. To a solution of 99-3 (700 mg, 1.285 mmol, 1 eq.) in EA (30 mL) was added Pd/C (1367.79 mg, 12.850 mmol, 10 eq.) under a nitrogen atmosphere in a 100 mL. The mixture was hydrogenated at room temperature for 2 h under a hydrogen atmosphere. The resulting mixture was filtered. The filter cake was washed with MeOH (4×50 mL). The filtrate was concentrated under reduced pressure. The residue was purified by reversed-phase flash chromatography with the following conditions: column: C18 silica gel; mobile phase: MeCN in water (0.1% FA), 35% to 45% gradient in 10 min; detector: UV 254 nm, to afford 99-4 (360 mg, 61.63%) as a brown solid.

Step 5. Synthesis of 1-(4-(3-fluoro-1-methoxycyclobutyl)pyridin-2-yl)-N-(1-methyl-1H-indazol-7-yl)-1H-pyrazole-4-sulfonamide (I-99). A solution of 99-4 (200 mg, 0.440 mmol, 1 eq.) and DAST (212.79 mg, 1.320 mmol, 3 eq.) in DCM (4 mL) was stirred for 3 h at −78° C. under a nitrogen atmosphere. The reaction was quenched with a saturated NaHCO₃ solution at 0° C. The aqueous layer was extracted with DCM (3×50 mL). The combined organic layers were washed with brine (10 mL) and dried over anhydrous Na₂SO₄. After filtration, the filtrate was concentrated under reduced pressure. The crude product (100 mg) was purified by reversed-phase flash chromatography with the following conditions: column: C18 silica gel; mobile phase: MeCN in water (0.1% FA), 30% to 50% gradient in 10 min; detector: UV 254 nm, to afford I-99 (33.6 mg, 16.66%) as an off-white solid. (ES, m/z): [M+H]+ 457; ¹H NMR: (400 MHz, DMSO-d₆) δ 2.56-2.62 (m, 2H), 2.87-2.99 (m, 2H), 3.02 (s, 3H), 4.29 (s, 3H), 5.31 (m, 1H), 6.72 (d, J=7.6 Hz, 1H), 6.98 (t, J=7.6 Hz, 1H), 7.49 (d, J=5.6 Hz, 1H), 7.70 (d, J=7.6 Hz, 1H), 7.90 (s, 1H), 8.04 (s, 1H), 8.09 (s, 1H), 8.56 (d, J=5.2 Hz, 1H), 8.83 (s, 1H), 10.05 (s, 1H).

### Example 283—Synthesis of 1-(4-Chloropyridin-2-yl)-N-(3-Fluoro-1-(Methyl-D3)-1H-Indazol-7-yl)-1H-Pyrazole-4-Sulfonamide (I-100)

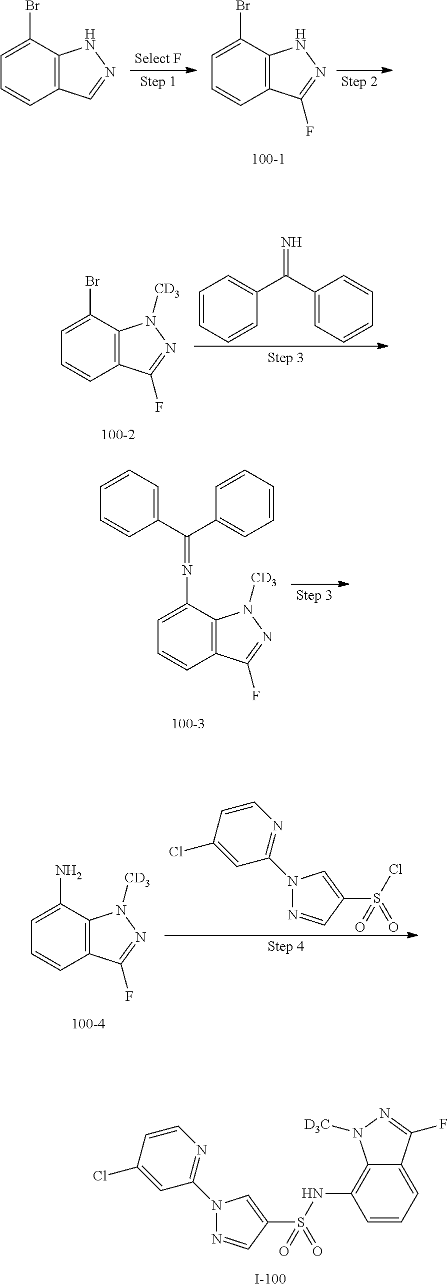

Step 1: Synthesis of 100-1. A mixture of 7-bromo-1H-indazole (5 g, 25. mmol, 1.00 eq.) and Selectfluor (13.4 g, 38.0 mmol, 1.50 eq.) in MeCN (20 mL) was stirred overnight at 60° C. under a nitrogen atmosphere. The reaction was quenched with water (50 mL) at room temperature. The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by silica gel column chromatography and eluted with PE/EA (12:1) to afford 7-bromo-3-fluoro-1H-indazole (2 g, 36%) as a yellow solid.

Step 2: Synthesis of 100-2. A mixture of 7-bromo-3-fluoro-1H-indazole (500 mg, 2.3 mmol, 1.00 eq.), Cs₂CO₃ (2270 mg, 6.97 mmol, 3.00 eq.) and CD₃I (505 mg, 3.48 mmol, 1.50 eq.) in THF (5 mL) was stirred for 2 h at room temperature under a nitrogen atmosphere. The reaction was quenched with water (20 mL) at room temperature. The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in 7-bromo-3-fluoro-1-(2H3)methylindazole (400 mg, 74%) as a yellow solid.

Step 3: Synthesis of 100-3. A mixture of 7-bromo-3-fluoro-1-(2H3) methylindazole (400 mg, 1.72 mmol, 1.00 eq.), diphenylmethanimine (312 mg, 1.72 mmol, 1.00 eq.), Pd₂(dba)₃ (315 mg, 0.345 mmol, 0.20 eq.), XantPhos (249 mg, 0.431 mmol, 0.25 eq.) and Cs₂CO₃ (1.68 mg, 5.17 mmol, 3.00 eq.) in dioxane (5 mL) was stirred overnight at 110° C. under a nitrogen atmosphere. The reaction was quenched with water (20 mL) at room temperature. The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in N-[3-fluoro-1-(2H3)methylindazol-7-yl]-1,1-diphenylmethanimine (400 mg, 69%) as a yellow solid.

Step 4: Synthesis of 100-4. To a stirred mixture of N-[3-fluoro-1-(2H3) methylindazol-7-yl]-1,1-diphenylmethanimine (300 mg, 0.903 mmol, 1.00 eq.) in DCM (2 mL) was added TFA (308 mg, 2.70 mmol, 3.00 eq.) dropwise at 0° C. under a nitrogen atmosphere. The resulting mixture was stirred for 2 h at room temperature under a nitrogen atmosphere. The resulting mixture was concentrated under reduced pressure. The residue was purified by reversed-phase flash chromatography with the following conditions: column: C18 silica gel; mobile phase: MeCN in water (0.1% NH₃·H₂O), 10% to 50% gradient in 10 min; detector: UV 254 nm. This resulted in 3-fluoro-1-(2H3)methylindazol-7-amine (100 mg, 65%) as a yellow solid.

Step 5: Synthesis of 1-(4-chloropyridin-2-yl)-N-(3-fluoro-1-(methyl-d3)-1H-indazol-7-yl)-1H-pyrazole-4-sulfonamide (I-100). A mixture of 3-fluoro-1-(2H3) methylindazol-7-amine (60 mg, 0.357 mmol, 1.00 eq.) and 1-[4-(trifluoromethyl) pyridin-2-yl] pyrazole-4-sulfonyl chloride (100 mg, 0.321 mmol, 0.90 eq.) in MeCN (5 mL) was stirred overnight at 50° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product (70 mg) was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 20% B to 55% B in 8 min; wavelength: 220 nm) to afford N-[3-fluoro-1-(2H3)methylindazol-7-yl]-1-[4-(trifluoromethyl)pyridin-2-yl]pyrazole-4-sulfonamide (24.7 mg, 15%) as an off-white solid. LCMS (ESI, m/z): [M+H]⁺ 410; ¹H NMR: (400 MHz, DMSO-d₆) δ 6.83 (d, J=7.2 Hz, 1H), 7.01 (t, J=7.6 Hz, 1H), 7.58 (d, J=7.6 Hz, 1H), 7.88 (d, J=5.2 Hz, 1H) 8.10 (s, 1H), 8.19 (s, 1H), 8.82 (d, J=5.2 Hz, 1H), 8.87 (s, 1H), 10.17 (s, 1H).

### Example 284—Synthesis of 1-(4-(3-Hydroxyoxetan-3-yl)Pyridin-2-yl)-N-(1-Methyl-1H-Indazol-7-yl)-1H-Pyrazole-4-Sulfonamide (I-101)

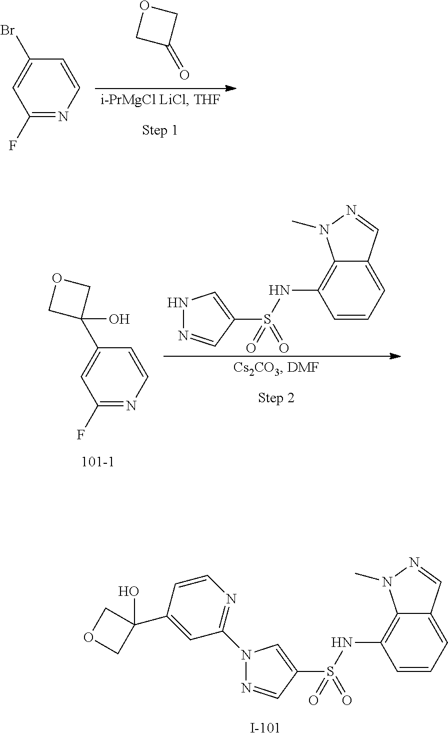 [SMILES: Fc1cc(Br)ccn1 ; O=C1COC1 ; OC1(c2ccnc(F)c2)COC1 ; Cn1ncc2cccc(NS(=O)(=O)c3cn[nH]c3)c21 ; Cn1ncc2cccc(NS(=O)(=O)c3cnn(-c4cc(C5(O)COC5)ccn4)c3)c21]

Step 1: Synthesis of 101-1. A solution of 4-bromo-2-fluoropyridine (500 mg, 2.841 mmol, 1 eq.) in THE (10 mL) was treated with butyllithium (3.4 mL, 8.500 mmol, 2.99 eq.) for 30 min at −78° C. under a nitrogen atmosphere followed by the addition of cyclobutanone (398.27 mg, 5.682 mmol, 2 eq.) dropwise at −78° C. The resulting mixture was stirred for 2.5 h at −78° C. under a nitrogen atmosphere. The reaction was quenched with sat. NH₄Cl (aq.) at room temperature. The resulting mixture was extracted with CH₂Cl₂ (3×30 mL). The combined organic layers were washed with brine (5 mL) and dried over anhydrous Na₂SO₄. After filtration, the filtrate was concentrated under reduced pressure. The residue was purified by reverse phase flash chromatography with the following conditions: column: XBridge Shield RP18 OBD column, 30*150 mm, 5 μm; mobile phase A: water (10 mmol/L NH₄CO₃+0.05% NH₃H₂O), mobile phase B: ACN; flow rate: 60 mL/min; gradient: 9% B to 33% B in 7 min; wavelength: 254 nm/220 nm; Rt(min): 7) to afford 101-1(300 mg, 60.63%) as a light yellow oil.

Step 2: Synthesis of 1-(4-(3-hydroxyoxetan-3-yl)pyridin-2-yl)-N-(1-methyl-1H-indazol-7-yl)-1H-pyrazole-4-sulfonamide (I-101). To a stirred solution of 101-1 (102.48 mg, 0.606 mmol, 1.2 eq.) and N-(1-methyl-1H-indazol-7-yl)-1H-pyrazole-4-sulfonamide (140 mg, 0.505 mmol, 1.00 eq.) in DMF (4 mL) was added Cs₂CO₃ (493.49 mg, 1.515 mmol, 3 eq.) in portions at room temperature under a nitrogen atmosphere. The resulting mixture was stirred for 2 h at 80° C. under a nitrogen atmosphere. The resulting mixture was extracted with EtOAc (2×20 mL). The combined organic layers were washed with brine, dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 40% gradient in 10 min; detector: UV 254 nm. The resulting mixture was concentrated under reduced pressure. This resulted in I-101 (28.6 mg, 13.08%) as a white solid. (ES, m/z): [M+H]⁺ 427; ¹H NMR: (400 MHz, DMSO-d₆) δ 4.29 (s, 3H), 4.69 (d, J=6.7 Hz, 2H), 4.85 (d, J=6.7 Hz, 2H), 6.71 (d, J=7.2 Hz, 1H), 6.87 (s, 1H), 6.97 (t, J=7.7 Hz, 1H), 7.74-7.66 (m, 2H), 8.04 (s, 1H), 8.09 (s, 1H), 8.21 (d, J=1.6 Hz, 1H), 8.55 (d, J=5.2 Hz, 1H), 8.82 (s, 1H), 10.04 (s, 1H).

### Example 285—Synthesis of 1-(4-Cyclopropylpyridin-2-yl)-N-(1-(2-Methoxyethyl)-1H-Indazol-7-yl)-1H-Pyrazole-4-Sulfonamide (I-102)

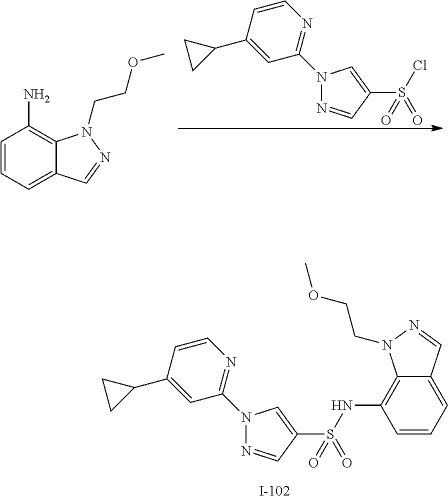

A solution of 1-(2-methoxyethyl)-1H-indazol-7-amine (150 mg, 0.784 mmol, 1.00 eq.) and 1-(4-cyclopropylpyridin-2-yl)pyrazole-4-sulfonyl chloride (244.8 mg, 0.862 mmol, 1.10 eq.) in pyridine (5 mL) was stirred for 2 h at 140° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃)⁺0.05% NH₃·H₂O, mobile phase B: ACN; flow rate: 60 mL/min; gradient: 32% B to 59% B in 7 min; wavelength: 254 nm/220 nm; Rt(min): 8.03 to afford 1-(4-cyclopropylpyridin-2-yl)-N-(1-(2-methoxyethyl)-1H-indazol-7-yl)-1H-pyrazole-4-sulfonamide (59.8 mg, 17.33%) as an off-white solid. (ES, m/z): [M+H]⁺ 439; ¹H NMR:(DMSO-d₆, 400 MHz) δ 0.83-0.89 (m, 2H), 1.08-1.15 (m, 2H), 2.04-2.12 (m, 1H), 3.14 (s, 3H), 3.74 (t, J=5.2 Hz, 2H), 4.53 (t, J=5.2 Hz, 2H), 6.96 (t, J=7.6 Hz, 1H), 7.10-7.16 (m, 2H), 7.45 (d, J=8.4 Hz, 1H), 7.59 (s, 1H), 8.09 (s, 1H), 8.28-8.32 (m, 2H), 8.99 (s, 1H), 10.18 (s, 1H).

### Example 286—Synthesis of N-(1-Methyl-1H-Indazol-7-yl)-1-(4-Morpholinopyridin-2-yl)-1H-Pyrazole-4-Sulfonamide (I-103)

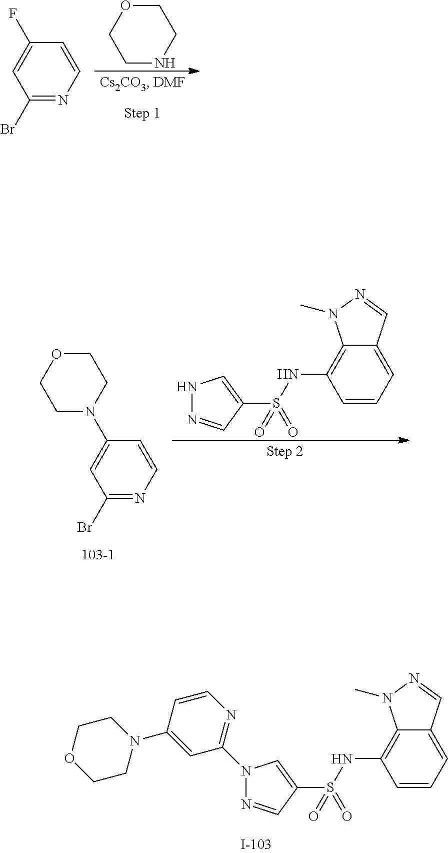 [SMILES: Fc1ccnc(Br)c1 ; C1COCCN1 ; Brc1cc(N2CCOCC2)ccn1 ; Cn1ncc2cccc(NS(=O)(=O)c3cn[nH]c3)c21 ; Cn1ncc2cccc(NS(=O)(=O)c3cnn(-c4cc(N5CCOCC5)ccn4)c3)c21]

Step 1: Synthesis of 103-1. A solution of 2-bromo-4-fluoropyridine (1 mL, 9.654 mmol, 1 eq.), Cs₂CO₃ (6.29 g, 19.308 mmol, 2 eq.) and morpholine (1.27 mL, 14.481 mmol, 1.5 eq.) in DMF (10 mL) was stirred for 3 h at 50° C. under a nitrogen atmosphere. The resulting mixture was extracted with EtOAc (3×10 mL). The combined organic layers were washed with brine (20 mL) and dried over anhydrous Na₂SO₄. After filtration, the filtrate was concentrated under reduced pressure. The crude product mixture was used in the next step directly without further purification. This resulted in 103-1 (2.7 g, 92.03%) as a white solid.

Step 2: Synthesis of N-(1-methyl-1H-indazol-7-yl)-1-(4-morpholinopyridin-2-yl)-1H-pyrazole-4-sulfonamide (103-2). To a stirred solution of N-(1-methylindazol-7-yl)-1H-pyrazole-4-sulfonamide (120 mg, 0.433 mmol, 1 eq.), 4-(2-bromopyridin-4-yl)morpholine (157.80 mg, 0.649 mmol, 1.5 eq.) and Cs₂CO₃ (422.99 mg, 1.299 mmol, 3 eq.) in DMF (5 mL) were added N1,N2-dimethylcyclohexane-1,2-diamine (61.56 mg, 0.433 mmol, 1 eq.) and CuI (41.21 mg, 0.216 mmol, 0.5 eq.) at room temperature under a nitrogen atmosphere. The resulting mixture was stirred overnight at 140° C. The resulting mixture was quenched with water (5 mL) and extracted with CH₂Cl₂ (3×10 mL). The combined organic layers were washed with water (3×10 mL) and dried over anhydrous Na₂SO₄. After filtration, the filtrate was concentrated under reduced pressure. The crude product (150 mg) was purified by prep-HPLC with the following conditions: column: XBridge Shield RP18 OBD column, 30*150 mm, 5 μm; mobile phase A: water (10 mmol/L NH₄CO₃+0.05% NH₃·H₂O), mobile phase B: ACN; flow rate: 60 mL/min; gradient: 20% B to 40% B in 7 min; wavelength: 254 nm/220 nm; Rt(min): 6.63) to afford N-(1-methyl-1H-indazol-7-yl)-1-(4-morpholinopyridin-2-yl)-1H-pyrazole-4-sulfonamide (90.3 mg, 47.34%) as a white solid. (ES, m/z): [M+H]⁺=440; ¹H NMR: ¹H NMR (400 MHz, DMSO-d₆) δ 3.40 (t, J=4.8 Hz, 4H), 3.73 (t, J=4.8 Hz, 4H), 4.28 (s, 3H), 6.70 (d, J=7.2 Hz, 1H), 6.91-6.99 (m, 2H), 7.33 (d, J=2.0 Hz, 1H), 7.68 (d, J=8.0 Hz, 1H), 7.94 (s, 1H), 8.08-8.11 (m, 2H), 8.73 (s, 1H), 9.96 (s, 1H).

### Example 287—Synthesis of N-(6-Fluoro-2-Methyl-2H-Indazol-7-yl)-1-(4-(Trifluoromethyl)Pyridin-2-yl)-1H-Pyrazole-4-Sulfonamide (I-104)

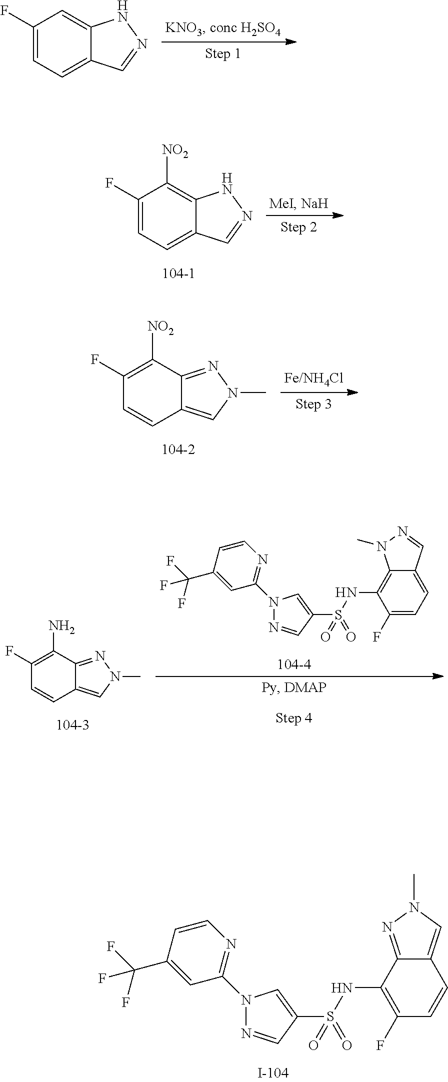 [SMILES: Fc1ccc2cn[nH]c2c1 ; O=[N+]([O-])c1c(F)ccc2cn[nH]c12 ; Cn1cc2ccc(F)c([N+](=O)[O-])c2n1 ; Cn1cc2ccc(F)c(N)c2n1 ; Cn1ncc2ccc(F)c(NS(=O)(=O)c3cnn(-c4cc(C(F)(F)F)ccn4)c3)c21 ; Cn1cc2ccc(F)c(NS(=O)(=O)c3cnn(-c4cc(C(F)(F)F)ccn4)c3)c2n1]

Step 1: Synthesis of 104-1. To a stirred mixture of 6-fluoro-1H-indazole (2 g, 14.692 mmol, 1 eq.) in fuming H₂SO₄ (10 mL) was added KNO₃ (1.78 g, 17.630 mmol, 1.2 eq.) at 0° C. under a nitrogen atmosphere. The resulting mixture was stirred overnight at room temperature. The reaction was quenched by the addition of water/ice (20 mL) at 0° C. The resulting mixture was extracted with CH₂Cl₂ (5×20 mL). The combined organic layers were washed with brine (2×30 mL) and dried over anhydrous Na₂SO₄. After filtration, the filtrate was concentrated under reduced pressure. The residue was purified by prep-TLC (CHCl3/MeOH 30:1) to afford 104-1 (800 mg, 30.06%) as a light yellow solid.

Step 2: Synthesis of 104-2. To a stirred solution of 104-1 (200 mg, 1.104 mmol, 1 eq.) in THE (10 mL) was added NaH (66.25 mg, 1.656 mmol, 1.5 eq., 60%) in portions at 0° C. under a nitrogen atmosphere. The resulting mixture was stirred for an additional 30 min at 0° C. To the above resulting solution was added methyl iodide (313.46 mg, 2.208 mmol, 2.0 eq.) dropwise at 0° C. under a nitrogen atmosphere. The resulting mixture was stirred for an additional 5 h at room temperature. The reaction was quenched by the addition of sat. NH₄Cl (aq.) (10 mL) at 0° C. The resulting mixture was extracted with CH₂Cl₂ (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄CO₃), 30% to 70% gradient in 10 min; detector: UV 220 nm to afford 104-2 (60 mg, 27.84%) as a brown solid.

Step 3: Synthesis of 104-3. To a stirred solution of 104-2 (55 mg, 0.282 mmol, 1 eq.) in MeOH (5 ml) were added Fe (78.69 mg, 1.410 mmol, 5 eq.) and NH₄Cl (75.38 mg, 1.410 mmol, 5 eq.) at room temperature under a nitrogen atmosphere. The resulting mixture was stirred for an additional 5 h at 80° C. The solids were collected by filtration and washed with MeOH (3×5 mL). The filtrate was concentrated under reduced pressure. The residue was purified by reversed-phase flash chromatography with the following conditions: column: C18 silica gel; mobile phase: MeCN in water (0.1% NH₄CO₃), 30% to 60% gradient in 10 min; detector: UV 220 nm. To afford 104-3 (40 mg, 85.93%) as a brown solid.

Step 4: Synthesis of N-(6-fluoro-2-methyl-2H-indazol-7-yl)-1-(4-(trifluoromethyl)pyridin-2-yl)-1H-pyrazole-4-sulfonamide (I-104). To a stirred solution of 104-3 (30 mg, 0.182 mmol, 1 eq.) and DMAP (4.44 mg, 0.036 mmol, 0.2 eq.) in pyridine (3 ml) was added 1-[4-(trifluoromethyl)pyridin-2-yl]pyrazole-4-sulfonyl chloride (56.61 mg, 0.182 mmol, 1.0 eq.) in portions at 0° C. under a nitrogen atmosphere. The resulting mixture was stirred for an additional 5 h at room temperature. The resulting mixture was concentrated under reduced pressure. The residue was purified by reversed-phase flash chromatography with the following conditions: column: C18 silica gel; mobile phase: MeCN in water (0.1% NH₄CO₃), 30% to 60% gradient in 10 min; detector: UV 220 nm. To afford crude product (20 mg) as a light yellow solid. The crude product (20 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 40% B to 51% B in 7 min; wavelength: 254 nm/220 nm; Rt(min): 6.5) to afford I-104 (6 mg, 7.44%) as an off-white solid. (ES, m/z): [M+H]+ 441.10; ¹H NMR: (400 MHz, DMSO-d6) 4.04 (s, 3H), 6.99 (dd, J=10.0, 9.6 Hz, 1H), 7.70 (dd, J=8.8, 4.8 Hz, 1H), 7.89 (d, J=4.8 Hz, 1H), 8.15 (s, 1H), 8.18 (s, 1H), 8.39 (s, 1H), 8.85 (d, J=5.2 Hz, 1H), 9.23 (s, 1H), δ 10.16 (s, 1H).

### Example 288—Synthesis of N-(3-Chloro-2-Methyl-2H-Indazol-7-yl)-1-(4-(Trifluoromethyl)Pyridin-2-yl)-1H-Pyrazole-4-Sulfonamide (I-105)

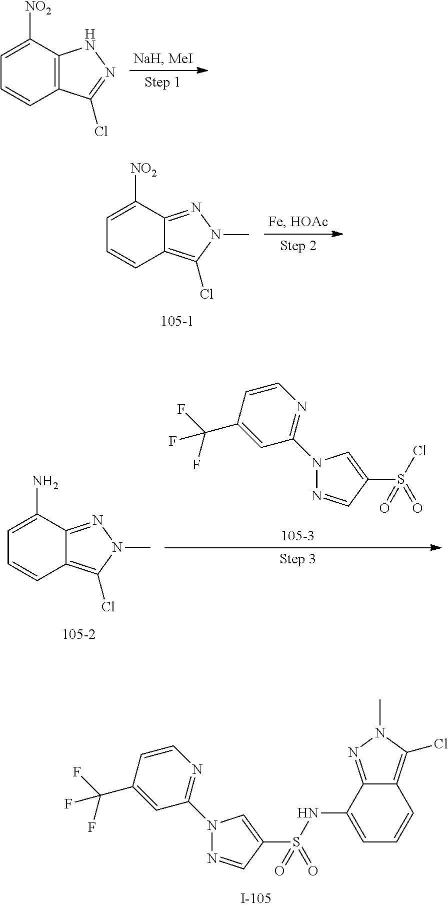 [SMILES: O=[N+]([O-])c1cccc2c(Cl)n[nH]c12 ; Cn1nc2c([N+](=O)[O-])cccc2c1Cl ; Cn1nc2c(N)cccc2c1Cl ; O=S(=O)(Cl)c1cnn(-c2cc(C(F)(F)F)ccn2)c1 ; Cn1nc2c(NS(=O)(=O)c3cnn(-c4cc(C(F)(F)F)ccn4)c3)cccc2c1Cl]

Step 1: Synthesis of 105-1. To a stirred solution of 3-chloro-7-nitro-1H-indazole (500 mg, 2.531 mmol, 1 eq.) and NaH (182.19 mg, 7.593 mmol, 3 eq.) in THE (20 mL) were added CH₃I (538.79 mg, 3.796 mmol, 1.5 eq.) in portions at 0° C. under an air atmosphere. The resulting mixture was stirred for an additional 2 h at room temperature. The reaction was quenched by the addition of water (20 mL) at room temperature. The resulting mixture was extracted with EtOAc (5×20 mL). The combined organic layers were washed with brine (20 mL) and dried over anhydrous Na₂SO₄. After filtration, the filtrate was concentrated under reduced pressure. This resulted in 105-1 (300 mg, 56.02%) as a yellow solid.

Step 2: Synthesis of 105-2. To a stirred mixture of 3-chloro-2-methyl-7-nitro-2H-indazole (150 mg, 0.709 mmol, 1 eq.) and Fe (197.93 mg, 3.545 mmol, 5 eq.) in EtOH (20 mL) were added NH₄Cl (189.58 mg, 3.545 mmol, 5 eq.) in portions at 80° C. under a nitrogen atmosphere. The resulting mixture was stirred for an additional 2 h at 80° C. The resulting mixture was filtered and the filter cake was washed with EtOH (5×1 mL). The filtrate was concentrated under reduced pressure. The residue was purified by reversed-phase flash chromatography with the following conditions: column: C18 silica gel; mobile phase: MeCN in water (0.1% NH₃·H₂O+10 mmol/L NH₄CO₃), 10% to 50% gradient in 10 min; detector: UV 254 nm. This resulted in 3-chloro-2-methyl-2H-indazol-7-amine (100 mg, 77.67%) as a yellow solid.

Step 3: Synthesis of N-(3-chloro-2-methyl-2H-indazol-7-yl)-1-(4-(trifluoromethyl) pyridin-2-yl)-1H-pyrazole-4-sulfonamide (I-105). To a stirred solution of 3-chloro-2-methyl-2H-indazol-7-amine (100 mg, 0.551 mmol, 1 eq.) in pyridine (20 mL) were added 1-(4-(trifluoromethyl)pyridin-2-yl)-1H-pyrazole-4-sulfonyl chloride (171.60 mg, 0.551 mmol, 1 eq.) in portions at 50° C. under a nitrogen atmosphere. The resulting mixture was stirred for an additional 2 h at 50° C. and then concentrated under vacuum. The residue was purified by reversed-phase flash chromatography with the following conditions: column: C18 silica gel; mobile phase: MeCN in water (0.1% NH₃·H₂O+10 mmol/L NH₄CO₃), 10% to 50% gradient in 10 min; detector: UV 254 nm. This resulted in N-(3-chloro-2-methyl-2H-indazol-7-yl)-1-(4-(trifluoromethyl)pyridin-2-yl)-1H-pyrazole-4-sulfonamide (50.9 mg, 20.07%) as a white solid. (ES, m/z): [M+H]+ 457; ¹H NMR: ¹H NMR (400 MHz, DMSO-d₆) δ 4.27 (s, 3H), 7.05 (t, J=8.0 Hz, 1H), 7.19 (d, J=7.6 Hz, 1H), 7.27 (d, J=7.2 Hz, 1H), 7.85 (d, J=4.8 Hz, 1H), 8.11 (s, 1H), 8.20 (s, 1H), 8.81 (d, J=5.2 Hz, 1H), 9.10 (s, 1H).

### Example 289—Synthesis of 1-(5-Cyclopropylpyridin-2-yl)-N-(3-Methyl-1H-Indazol-4-yl)-1H-Pyrazole-4-Sulfonamide (I-106)

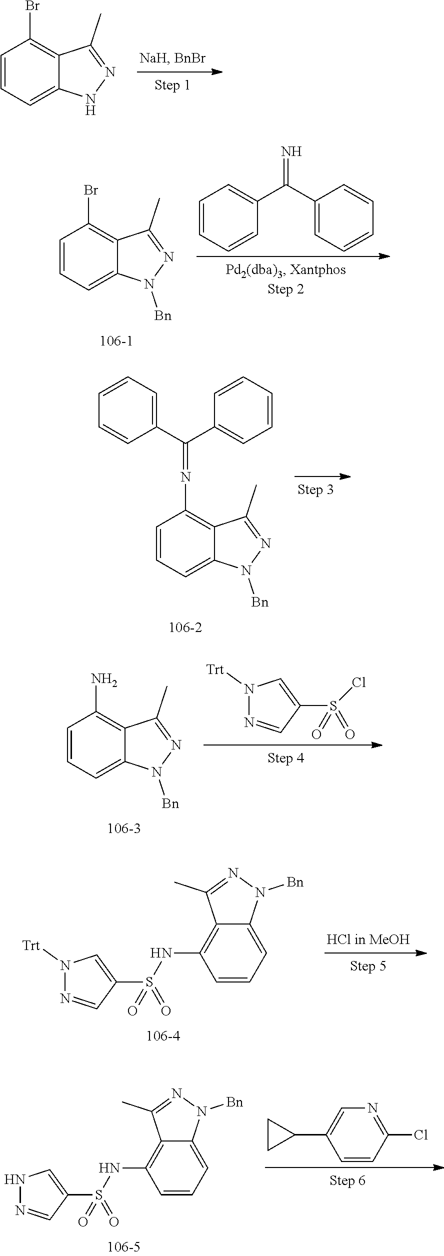

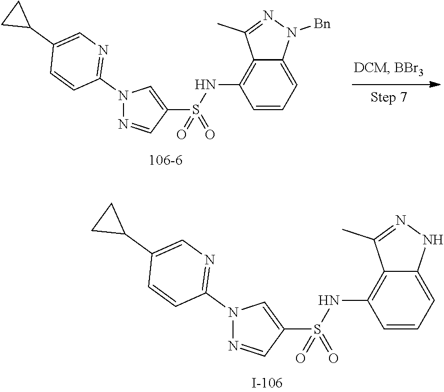 [SMILES: Cc1nn(Cc2ccccc2)c2cccc(NS(=O)(=O)c3cnn(-c4ccc(C5CC5)cn4)c3)c12 ; Cc1n[nH]c2cccc(NS(=O)(=O)c3cnn(-c4ccc(C5CC5)cn4)c3)c12]

Step 1: Synthesis of 106-1. A solution of 4-bromo-3-methyl-1H-indazole (4 g, 18.952 mmol, 1 eq.) in DMF (20 mL) was treated with NaH (909.61 mg, 37.904 mmol, 2 eq.) for 30 min at 0° C. under a nitrogen atmosphere followed by the addition of BnBr (4.86 g, 28.428 mmol, 1.5 eq.) dropwise at room temperature. The resulting mixture was stirred for an additional 2 h at room temperature. The resulting mixture was quenched with H₂O (100 mL) and extracted with EtOAc (3×100 mL). The combined organic layers were washed with brine (3×10 mL) and dried over anhydrous Na₂SO₄. After filtration, the filtrate was concentrated under reduced pressure. This resulted in 106-1 (10 g, 87.60%) as a yellow oil.

Step 2: Synthesis of 106-2. To a stirred solution of 1-benzyl-4-bromo-3-methylindazole (1 g, 3.320 mmol, 1 eq.), K₂CO₃ (1.38 g, 9.960 mmol, 3 eq.) and diphenylmethanimine (902.62 mg, 4.980 mmol, 1.5 eq.) in DMF (6 mL) were added Pd₂(dba)₃ (608.08 mg, 0.664 mmol, 0.2 eq.) and Xantphos (384.23 mg, 0.664 mmol, 0.2 eq.) at room temperature under a nitrogen atmosphere. The resulting mixture was stirred overnight at 140° C. The resulting mixture was quenched with water (10 mL) and extracted with CH₂Cl₂ (3×10 mL). The combined organic layers were washed with water (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 0% to 100% gradient in 20 min; detector: UV 254 nm. This resulted in 106-2 (1 g, 80%) as a yellow oil.

Step 3: Synthesis of 106-3. A solution of N-(1-benzyl-3-methylindazol-4-yl)-1,1-diphenylmethanimine (1 g, 2.491 mmol, 1 eq.), AcONa (306.47 mg, 3.737 mmol, 1.5 eq.) and hydroxylamine hydrochloride (259.61 mg, 3.737 mmol, 1.5 eq.) in MeOH (6 mL) was stirred for 3 h at 50° C. under a nitrogen atmosphere. The resulting mixture was diluted with H₂O (20 mL) and extracted with CH₂Cl₂ (3×10 mL). The combined organic layers were washed with brine (30 mL). After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 70% gradient in 25 min; detector: UV 254 nm, to afford 106-3 (600 mg, 91.37%) as a yellow oil.

Step 4: Synthesis of 106-4. A solution of 1-benzyl-3-methylindazol-4-amine (200 mg, 0.801 mmol, 1 eq., 95%) and 1-(triphenylmethyl)pyrazole-4-sulfonyl chloride (654.77 mg, 1.602 mmol, 2 eq.) in pyridine (5 mL) was stirred for 3 h at 90° C. under a nitrogen atmosphere. The resulting mixture was concentrated and diluted with EtOAc (20 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 15% to 80% gradient in 30 min; detector: UV 254 nm. This resulted in 106-4 (300 mg, 43.02%) as a grey solid.

Step 5: Synthesis of 106-5. A solution of N-(1-benzyl-3-methylindazol-4-yl)-1-(triphenylmethyl)pyrazole-4-sulfonamide (1.1 g, 1.804 mmol, 1 eq.) in HCl/MeOH(4M, 10 mL) was stirred for 3 h at room temperature under a nitrogen atmosphere. The crude product mixture was concentrated under reduced pressure. This resulted in 106-5 (1.1 g, 82.97%) as a brown yellow solid which was used in the next step directly without further purification.

Step 6: Synthesis of 106-6. To a stirred solution of N-(1-benzyl-3-methylindazol-4-yl)-1H-pyrazole-4-sulfonamide (400 mg, 0.544 mmol, 1 eq., 50%), 2-chloro-5-cyclopropylpyridine (125.42 mg, 0.816 mmol, 1.5 eq.) and Cs₂CO₃ (0.53 g, 1.632 mmol, 3 eq.) in DMF (10 mL) were added N1, N2-dimethylcyclohexane-1,2-diamine (77.43 mg, 0.544 mmol, 1 eq.) and CuI (51.83 mg, 0.272 mmol, 0.5 eq.) at room temperature under a nitrogen atmosphere. The resulting mixture was stirred overnight at 140° C. The resulting mixture was diluted with H₂O (20 mL) and extracted with CH₂Cl₂ (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 20% to 80% gradient in 20 min; detector: UV 254 nm. This resulted in 106-6 (150 mg, 51.18%) as a dark grey solid.

Step 7: Synthesis of 1-(5-cyclopropylpyridin-2-yl)-N-(3-methyl-1H-indazol-4-yl)-1H-pyrazole-4-sulfonamide (I-106). A solution of N-(1-benzyl-3-methylindazol-4-yl)-1-(5-cyclopropylpyridin-2-yl)pyrazole-4-sulfonamide (140 mg, 0.289 mmol, 1 eq.) and BBr3 (3 mL) in DCM (3 mL) was stirred overnight at 50° C. under a nitrogen atmosphere. The resulting mixture was quenched with H₂O (20 mL) and extracted with CH₂Cl₂ (3×20 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 15% to 60% gradient in 30 min; detector: UV 254 nm. The crude product (50 mg) was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 20% B to 47% B in 7 min; wavelength: 254 nm/220 nm; Rt(min): 8.02) to afford I-106 (13.6 mg, 11.81%) as a white solid. (ES, m/z): [M+H]⁺ 395; ¹H NMR: ¹H NMR (400 MHz, DMSO-d₆) δ 0.77-0.80 (m, 2H), 1.02-1.05 (m, 2H), 2.02-2.05 (m, 1H), 2.59 (s, 3H), 6.56 (d, J=7.2 Hz, 1H), 7.17 (t, J=8.0 Hz, 1H), 7.31 (d, J=7.2 Hz, 1H), 7.69 (d, J=8.8 Hz, 1H), 7.82 (d, J=8.8 Hz, 1H), 7.96 (s, 1H), 8.32 (s, 1H), 8.72 (s, 1H), 9.85 (s, 1H), 12.69 (s, 1H).

### Example 290—Synthesis of 1-(4-(1-Hydroxycyclobutyl)Pyridin-2-yl)-N-(1-Methyl-1H-Indazol-7-yl)-1H-Pyrazole-4-Sulfonamide (I-107)

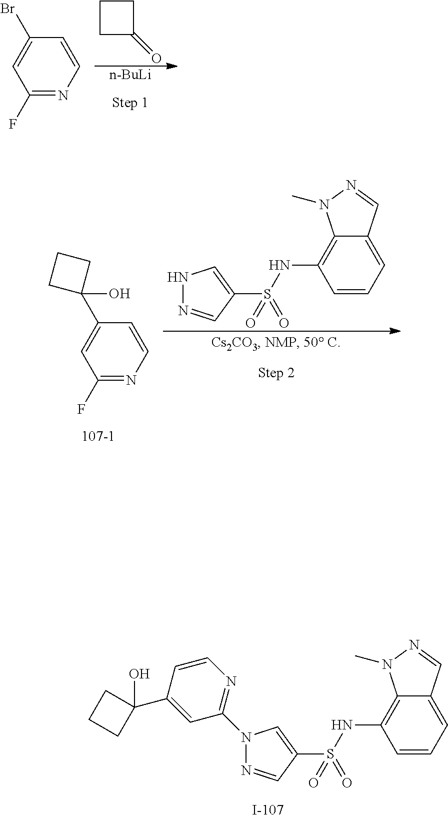

Step 1: Synthesis of 107-1. A solution of 4-bromo-2-fluoropyridine (1.7 g, 9.660 mmol, 1 eq.) in THE (10 mL) was treated with butyllithium (11.59 mL, 28.980 mmol, 3 eq.) for 30 min at −78° C. under a nitrogen atmosphere followed by the addition of cyclobutanone (1.35 g, 19.320 mmol, 2 eq.) dropwise at −78° C. The resulting mixture was stirred for 2.5 h at −78° C. to room temperature under a nitrogen atmosphere. The reaction was quenched with sat. NH₄Cl (aq.) at room temperature. The resulting mixture was diluted with water (30 mL). The resulting mixture was extracted with CH₂Cl₂ (3×30 mL). The combined organic layers were washed with brine (50 mL) and dried over anhydrous Na₂SO₄. After filtration, the filtrate was concentrated under reduced pressure. The residue was purified by reverse phase flash chromatography with the following conditions: column: Xbridge Shield RP18 OBD column, 30*150 mm, 5 μm; mobile phase A: water (10 mmol/L NH₄CO₃+0.05% NH₃H₂O), mobile phase B: ACN; flow rate: 60 mL/min; gradient: 9% B to 33% B in 7 min; wavelength: 254 nm/220 nm; Rt(min): 7) to afford 1-(2-fluoropyridin-4-yl)cyclobutan-1-ol (700 mg, 43.35%) as a light yellow oil.

Step 2: Synthesis of 1-(4-(1-hydroxycyclobutyl)pyridine-2-yl)-N-(1-methyl-1H-indazol-7-yl)-1H-pyrazole-4-sulfonamide (I-107). A mixture of 1-(2-fluoropyridin-4-yl)cyclobutan-1-ol (300 mg, 1.794 mmol, 1 eq.), N-(1-methylindazol-7-yl)-1H-pyrazole-4-sulfonamide (418 mg, 1.507 mmol, 0.84 eq.), and Cs₂CO₃ (1800 mg, 5.525 mmol, 3.08 eq.) in NMP (12 mL) was stirred for 3 days at 50° C. under a nitrogen atmosphere. The solution was dissolved in water (50 mL). The resulting mixture was extracted with EtOAc (3×25 mL). The combined organic layers were washed with brine (50 mL) and dried over anhydrous Na₂SO₄. After filtration, the filtrate was concentrated under reduced pressure. The crude product (200 mg) was purified by prep-HPLC with the following conditions: column: YMC-Actus Triant C18 ExRs column, 30*150 mm, 5 μm; mobile phase A: water (10 mmol/L NH₄CO₃+0.05% NH₃H₂O), mobile phase B: 20 mm NaOH+10% ACN; flow rate: 60 mL/min; gradient: 10% B to 35% B in 10 min; wavelength: 220 nm; Rt(min): 9.95) to afford 1-[4-(1-hydroxycyclobutyl) pyridine-2-yl]-N-(1-methylindazol-7-yl)pyrazole-4-sulfonamide (22.5 mg, 2.87%) as an off-white solid. (ES, m/z): [M+H]⁺ 425; ¹H NMR: (400 MHz, DMSO-d₆) δ 1.65-1.93 (m, 1H), 1.97-2.10 (m, 1H), 2.30-2.47 (m, 4H), 4.28 (s, 3H), 5.99 (s, 1H), 6.72 (d, J=7.2 Hz, 1H), 6.97 (t, J=7.6 Hz, 1H), 7.58 (d, J=4.8 Hz, 1H), 7.69 (d, J=7.6 Hz, 1H), 8.01 (s, 1H), 8.08 (s, 2H), 8.47 (d, J=4.8 Hz, 1H), 8.79 (s, 1H), 10.00 (s, 1H).

### Example 291—Synthesis of N-(1-(Difluoromethyl)-1H-Indazol-7-yl)-1-(4-(Difluoromethyl)Pyridin-2-yl)-1H-Pyrazole-4-Sulfonamide (I-108)

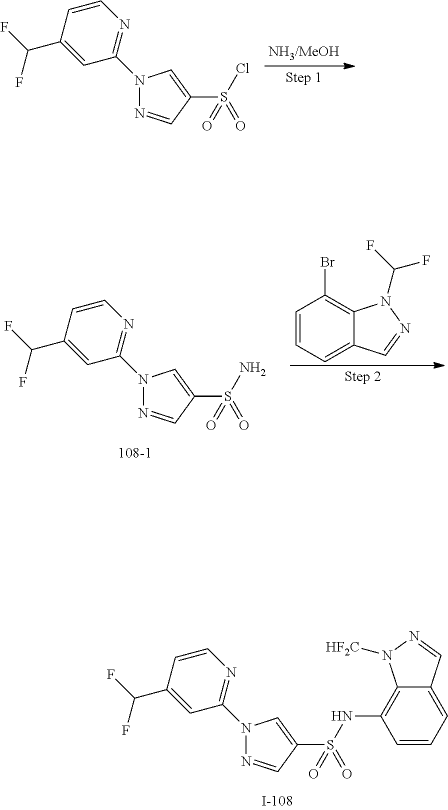

Step 1: Synthesis of 108-1. A solution of 1-[4-(difluoromethyl)pyridin-2-yl]pyrazole-4-sulfonyl chloride (100 mg, 0.341 mmol, 1 eq.) in NH₃/MeOH (3 mL) was stirred for 2 h at room temperature. The resulting mixture was concentrated under reduced pressure. The crude product was used in the next step directly without further purification. This resulted in 1-[4-(difluoromethyl)pyridin-2-yl]pyrazole-4-sulfonamide (90 mg, 85.77%) as a brown solid.

Step 2: Synthesis of N-(1-(difluoromethyl)-1H-indazol-7-yl)-1-(4-(difluoromethyl) pyridin-2-yl)-1H-pyrazole-4-sulfonamide (I-108). A solution of 1-[4-(difluoromethyl)pyridin-2-yl]pyrazole-4-sulfonamide (70 mg, 0.255 mmol, 1 eq.), 7-bromo-1-(difluoromethyl)indazole (94.58 mg, 0.383 mmol, 1.5 eq.), Gphos (20.63 mg, 0.038 mmol, 0.15 eq.), Gphos Pd G6 TES (36.16 mg, 0.038 mmol, 0.15 eq.) and Cs₂CO₃ (249.49 mg, 0.765 mmol, 3 eq.) in dioxane (1.5 mL) was stirred for 16 h at 110° C. under a nitrogen atmosphere. The residue was dissolved in water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-[1-(difluoromethyl)indazol-7-yl]-1-[4-(difluoromethyl)pyridin-2-yl]pyrazole-4-sulfonamide (9.5 mg, 8.42%) as a white solid. (ES, m/z): [M+H]⁺ 441; ¹H NMR: (400 MHz, methanol-d₄) δ 6.88 (t, J=15.2 Hz, 1H), 7.10 (t, J=7.6 Hz, 1H), 7.42-7.50 (m, 3H), 7.74 (t, J=19.6 Hz, 1H), 7.96 (s, 1H), 8.03 (s, 1H), 8.55-9.57 (m, 2H), 8.99 (s, 1H).

### Example 292—Synthesis of 1-(4-(2,5-Dihydrofuran-3-yl)Pyridin-2-yl)-N-(1-Methyl-1H-Indazol-7-yl)-1H-Pyrazole-4-Sulfonamide (I-109)

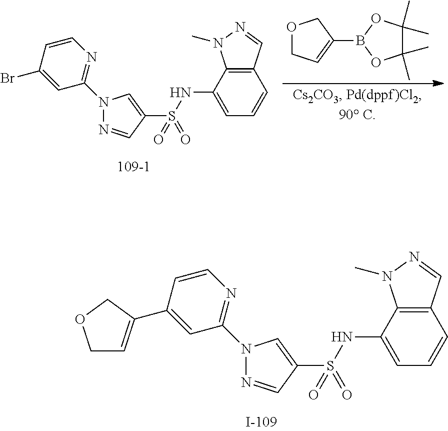

A mixture of 109-1 (50 mg, 0.115 mmol, 1 eq.), Pd(PPh₃)₄(13.34 mg, 0.012 mmol, 0.1 eq.), Na₂CO₃ (48.92 mg, 0.460 mmol, 4 eq.) and 2-(2,5-dihydrofuran-3-yl)-4,4,5,5-tetramethyl-1,3,2-dioxaborolane (45.25 mg, 0.230 mmol, 2 eq.) in 1,4-dioxane(5 ml) was stirred for 3 h at 80° C. under a nitrogen atmosphere. The resulting mixture was filtered and the filter cake was washed with 1,4-dioxane (3×30 mL). The filtrate was concentrated under reduced pressure. The residue was purified by reversed-phase flash chromatography with the following conditions: column: C18 silica gel; mobile phase: MeCN in water (10 mmol/L NH₄CO₃), 30% to 50% gradient in 15 min; detector: UV 254 nm. This resulted in 1-109 (18 mg, 36.70%) as a white solid. (ES, m/z): [M+H]⁺ 423; ¹H NMR:(400 MHz, DMSO-d₆) δ 4.32 (s, 3H), 4.78-4.81 (m, 2H), 4.96-4.98 (m, 2H), 6.75 (d, J=7.6 Hz, 1H), 6.90 (t, J=8.0 Hz, 1H), 7.04-7.05 (m, 1H), 7.50-7.52 (m, 2H), 7.84 (s, 1H), 7.99 (s, 2H), 8.49 (d, J=5.2 Hz, 1H), 8.74 (s, 1H).

### Example 293—Synthesis of 1-(4-Chloropyridin-2-yl)-N-(1-(Difluoromethyl)-1H-Indazol-7-yl)-1H-Pyrazole-4-Sulfonamide (I-110)

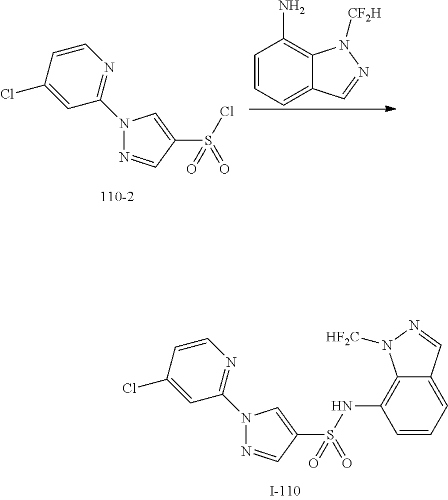

A mixture of 1-(4-chloropyridin-2-yl)pyrazole-4-sulfonamide (50 mg, 0.193 mmol, 1 eq.), 7-bromo-1-(difluoromethyl)indazole (71.63 mg, 0.289 mmol, 1.5 eq.), Gphos (15.62 mg, 0.029 mmol, 0.15 eq.), Cs₂CO₃ (188.93 mg, 0.579 mmol, 3 eq.) and Gphos Pd G6 TES (27.38 mg, 0.029 mmol, 0.15 eq.) in dioxane (1.5 mL) was stirred for 16 h at 110° C. under a nitrogen atmosphere. The reaction mixture was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(4-chloropyridin-2-yl)-N-[1-(difluoromethyl)indazol-7-yl]pyrazole-4-sulfonamide (6.7 mg, 8.14%) as a white solid. (ES, m/z): [M+H]⁺ 425; ¹H NMR: (400 MHz, DMSO-d₆) δ 7.09 (t, J=7.6 Hz, 1H), 7.27 (d, J=7.2 Hz, 1H), 7.53 (d, J=8.0 Hz, 1H), 7.60-7.61 (m, 1H), 7.94-8.24 (m, 3H), 8.49 (d, J=5.2 Hz, 1H), 8.87 (s, 1H), 9.01 (s, 1H), 10.58 (s, 1H).

### Example 294—Synthesis of N-(3-Chloro-1-Methyl-1H-Indazol-7-yl)-1-(4-(Trifluoromethyl)Pyridin-2-yl)-1H-Pyrazole-4-Sulfonamide (I-111)

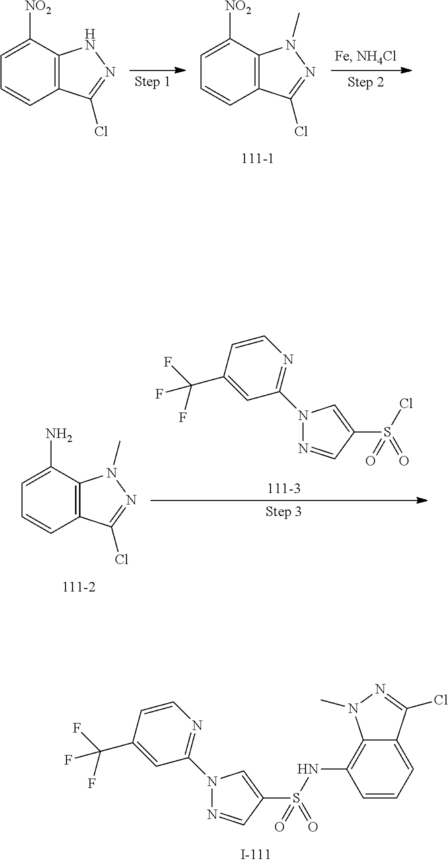 [SMILES: O=[N+]([O-])c1cccc2c(Cl)n[nH]c12 ; Cn1nc(Cl)c2cccc([N+](=O)[O-])c21 ; Cn1nc(Cl)c2cccc(N)c21 ; O=S(=O)(Cl)c1cnn(-c2cc(C(F)(F)F)ccn2)c1 ; Cn1nc(Cl)c2cccc(NS(=O)(=O)c3cnn(-c4cc(C(F)(F)F)ccn4)c3)c21]

Step 1: Synthesis of 111-1. To a stirred solution of 3-chloro-7-nitro-1H-indazole (400 mg, 2.024 mmol, 1 eq.) and Cs₂CO₃ (1319.24 mg, 4.048 mmol, 2 eq.) in DMF (20 mL) were added Mel (431.03 mg, 3.036 mmol, 1.5 eq.) dropwise at room temperature under a nitrogen atmosphere. The resulting mixture was stirred for 2 h at room temperature. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. This resulted in 111-1 (350 mg, 81.70%) as a yellow solid.

Step 2: Synthesis of 111-2. To a stirred mixture of 111-1 (300 mg, 1.418 mmol, 1 eq.) and Fe (395.86 mg, 7.090 mmol, 5 eq.) in EtOH (2 mL) was added NH₄Cl (379.16 mg, 7.090 mmol, 5 eq.) in portions at 80° C. under a nitrogen atmosphere. The resulting mixture was stirred for 2 h at 80° C. The resulting mixture was filtered and the filter cake was washed with EtOH (5×1 mL). The filtrate was concentrated under reduced pressure. The residue was purified by reversed-phase flash chromatography with the following conditions: column: C18 silica gel; mobile phase: MeCN in water (0.1% NH₃·H₂O+10 mmol/L NH₄CO₃), 10% to 50% gradient in 10 min; detector: UV 254 nm. This resulted in 111-2 (250 mg, 97.09%) as a yellow solid.

Step 3: Synthesis of N-(3-chloro-1-methyl-1H-indazol-7-yl)-1-(4-(trifluoromethyl) pyridin-2-yl)-1H-pyrazole-4-sulfonamide (I-111). To a stirred solution of 111-2 (120 mg, 0.385 mmol, 1 eq.) and 3-chloro-1-methylindazol-7-amine (76.92 mg, 0.424 mmol, 1.1 eq.) in pyridine (10 mL) was added DMAP (9.41 mg, 0.077 mmol, 0.2 eq.) at 80° C. under a nitrogen atmosphere. The resulting mixture was stirred for 2 h at 80° C. and then concentrated under vacuum. The crude product (120 mg) was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 27% B to 55% B in 7 min; wavelength: 254 nm/220 nm; Rt(min): 7.68) to afford N-(3-chloro-1-methyl-1H-indazol-7-yl)-1-(4-(trifluoromethyl)pyridin-2-yl)-1H-pyrazole-4-sulfonamide (4.7 mg, 2.65%) as a yellow solid. (ES, m/z): [M+H]⁺ 457; ¹H NMR: 1H NMR (400 MHz, DMSO-d₆) δ 4.27 (s, 3H), 6.86 (d, J=7.2 Hz, 1H), 7.06 (t, J=8.0 Hz, 1H), 7.48 (s, 1H), 7.86 (d, J=5.2 Hz, 1H), 8.07 (s, 1H), 8.18 (s, 1H), 8.81-8.84 (m, 2H), 10.18 (s, 1H).

### Example 295—Synthesis of 1-(4-(1,3-Difluorocyclobutyl)Pyridin-2-yl)-N-(1-Methyl-1H-Indazol-7-yl)-1H-Pyrazole-4-Sulfonamide (I-112)

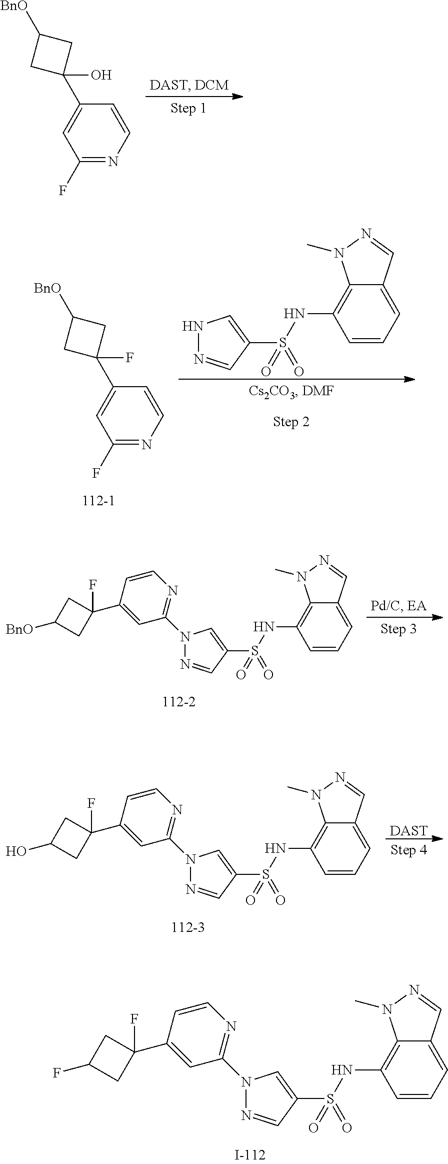

Step 1: Synthesis of 112-1. A solution of 3-(benzyloxy)-1-(2-fluoropyridin-4-yl)cyclobutan-1-ol (1.5 g, 5.488 mmol, 1 eq.) and DAST (2.65 g, 16.464 mmol, 3 eq.) in DCM (30 mL) was stirred for 3 h at −78° C. under a nitrogen atmosphere. The reaction was quenched with saturated NaHCO₃ at 0° C. The aqueous layer was extracted with DCM (3×100 mL). The combined organic layers were washed with brine (20 mL) and dried over anhydrous Na₂SO₄. After filtration, the filtrate was concentrated under reduced pressure. The residue was purified by silica gel column chromatography and eluted with PE/EA (5:1) to afford 112-1 (1.2 g, 79.42%) as a yellow oil.

Step 2: Synthesis of 112-2. To a stirred solution of 112-1 (300 mg, 1.090 mmol, 1 eq.) and N-(1-methylindazol-7-yl)-1H-pyrazole-4-sulfonamide (302.18 mg, 1.090 mmol, 1 eq.) in DMF (6 mg) was added Cs₂CO₃ (1065.16 mg, 3.270 mmol, 3 eq.) in portions at room temperature. The resulting mixture was stirred for 12 h at 80° C. under a nitrogen atmosphere. The reaction was quenched by the addition of water (10 mL) at 0° C. The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 65% to 75% gradient in 10 min; detector: UV 254 nm, to afford 112-2 (500 mg, 86.15%) as a brown solid.

Step 3: Synthesis of 112-3. To a solution of 112-2 (250 mg, 0.469 mmol, 1 eq.) in EA (20 mL) was added Pd/C (499.54 mg, 4.690 mmol, 10 eq.) under a nitrogen atmosphere. The mixture was hydrogenated at room temperature for 2 h under a hydrogen atmosphere. The resulting mixture was filtered. The filter cake was washed with MeOH (4×50 mL). The filtrate was concentrated under reduced pressure. The residue was purified by reversed-phase flash chromatography with the following conditions: column: C18 silica gel; mobile phase: MeCN in water (0.1% FA), 35% to 45% gradient in 10 min; detector: UV 254 nm, to afford 112-3 (100 mg, 48.15%) as a yellow solid.

Step 4: Synthesis of 1-(4-(1,3-difluorocyclobutyl)pyridin-2-yl)-N-(1-methyl-11H-indazol-7-yl)-1H-pyrazole-4-sulfonamide (I-112). A solution of 112-3 (100 mg, 0.226 mmol, 1 eq.) in DCM (5 mL) was stirred for 2 min at −78° C. under a nitrogen atmosphere followed by the addition of DAST (109.29 mg, 0.678 mmol, 3 eq.) dropwise at −78° C. The mixture was stirred for 1 h at −50° C. under a nitrogen atmosphere. The reaction was quenched by the addition of sat. NaHCO₃ (aq.) (2 mL) at 0° C. The aqueous layer was extracted with CH₂Cl₂ (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 60% gradient in 10 min; detector: UV 254 nm, to afford 1-(4-(1,3-difluorocyclobutyl)pyridin-2-yl)-N-(1-methyl-1H-indazol-7-yl)-1H-pyrazole-4-sulfonamide (32.4 mg, 30.80%) as an off-white solid. (ES, m/z): [M+H]+ 445; ¹H NMR: (400 MHz, DMSO-d₆) δ 2.90-3.04 (m, 2H), 3.12-3.24 (m, 2H), 4.28 (s, 3H), 5.19-5.34 (m, 1H), 6.71 (d, J=7.2 Hz, 1H), 6.97 (t, J=7.6 Hz, 1H), 7.53 (d, J=4.8 Hz, 1H), 7.71 (d, J=8.0 Hz, 1H), 7.94 (s, 1H), 8.05 (s, 1H), 8.09 (s, 1H), 8.58 (d, J=5.2 Hz, 1H), 8.83 (s, 1H), 10.05 (s, 1H).

### Example 296—Synthesis of 1-(4-(2-Fluoropropan-2-yl)Pyridin-2-yl)-N-(1-Methyl-1H-Indazol-7-yl)-1H-Pyrazole-4-Sulfonamide (I-113)

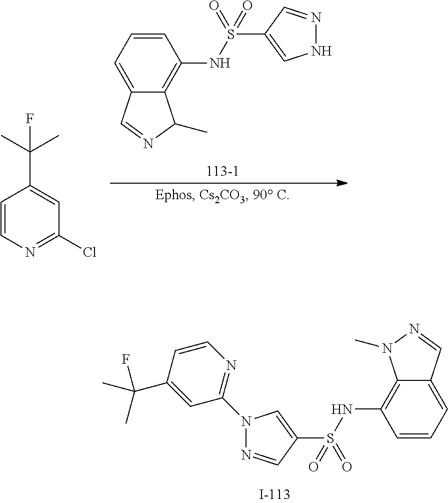 [SMILES: CC(C)(F)c1ccnc(Cl)c1 ; CC1N=Cc2cccc(NS(=O)(=O)c3cn[nH]c3)c21 ; Cn1ncc2cccc(NS(=O)(=O)c3cnn(-c4cc(C(C)(C)F)ccn4)c3)c21]

A solution of 2-chloro-4-(2-fluoropropan-2-yl) pyridine (70 mg, 0.403 mmol, 1 eq.) in dioxane (3 mL) was treated with 113-3 (133.68 mg, 0.484 mmol, 1.2 eq.) for 2 min at room temperature under a nitrogen atmosphere followed by the addition of EPhos (43.12 mg, 0.081 mmol, 0.2 eq.) and Cs₂CO₃ (394.09 mg, 1.209 mmol, 3 eq.) in portions at 90° C. The resulting mixture was stirred overnight. The resulting mixture was concentrated under reduced pressure. The residue was purified by reversed-phase flash chromatography with the following conditions: column: C18 silica gel; mobile phase: MeCN in water (0.1% FA), 30% to 70% gradient in 20 min; detector: UV 254 nm. This resulted in 1-(4-(2-fluoropropan-2-yl)pyridin-2-yl)-N-(1-methyl-1H-indazol-7-yl)-1H-pyrazole-4-sulfonamide (26.9 mg, 16.00%) as a white solid. (ES, m/z): [M+H]⁺ 415; ¹H NMR: (400 MHz, DMSO-d₆) δ 1.71 (d, J=22.4 Hz, 6H), 4.29 (s, 3H), 6.71 (d, J=7.2 Hz, 1H), 6.96 (t, J=7.6 Hz, 1H), 7.51 (d, J=4.0 Hz, 1H), 7.66 (d, J=7.6 Hz, 1H), 7.97 (s, 1H), 8.03 (s, 1H), 8.07 (s, 1H), 8.53 (d, J=4.4 Hz, 1H), 8.80 (s, 1H), 10.22 (s, 1H).

### Example 297—Synthesis of 1-(4-(2-Methoxypropan-2-yl)Pyridin-2-yl)-N-(1-Methyl-1H-Indazol-7-yl)-1H-Pyrazole-4-Sulfonamide (I-114)

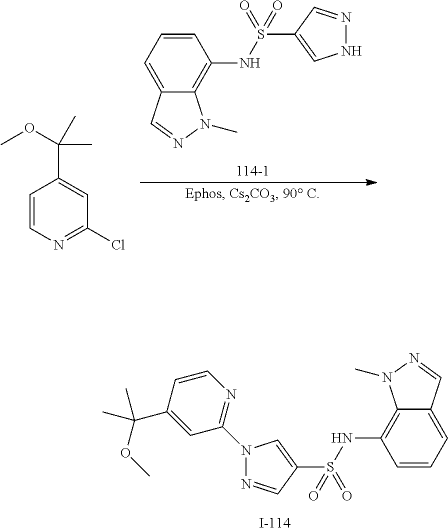 [SMILES: COC(C)(C)c1ccnc(Cl)c1 ; Cn1ncc2cccc(NS(=O)(=O)c3cn[nH]c3)c21 ; COC(C)(C)c1ccnc(-n2cc(S(=O)(=O)Nc3cccc4cnn(C)c34)cn2)c1]

A solution of 2-chloro-4-(2-methoxypropan-2-yl)pyridine (100 mg, 0.539 mmol, 1 eq.) in dioxane (4 mL) was treated with 114-1 (179.24 mg, 0.647 mmol, 1.2 eq.) for 2 min at room temperature under a nitrogen atmosphere followed by the addition of Ephos (57.61 mg, 0.108 mmol, 0.2 eq.) and Cs₂CO₃ (526.51 mg, 1.617 mmol, 3 eq.) in portions at 90° C. The resulting mixture was concentrated under reduced pressure. The residue was purified by reversed-phase flash chromatography with the following conditions: column: C18 silica gel; mobile phase: MeCN in water (0.1% FA), 30% to 70% gradient in 10 min; detector: UV 254 nm. This resulted in 1-(4-(2-methoxypropan-2-yl)pyridine-2-yl)-N-(1-methyl-1H-indazol-7-yl)-1H-pyrazole-4-sulfonamide (25.2 mg, 10.83%) as a white solid. (ES, m/z): [M+H]⁺ 427; ¹H NMR: (400 MHz, DMSO-d₆) δ 1.25 (s, 6H), 3.11 (s, 3H), 4.30 (s, 3H), 6.74 (s, 1H), 6.97 (s, 1H), 7.48 (s, 1H), 7.66 (s, 1H), 7.97 (s, 1H), 8.03 (s, 1H), 8.08 (s, 1H), 8.49 (s, 1H), 8.80 (s, 1H), 10.17 (s, 1H).

### Example 298—Synthesis of 1-(4-(Tert-Butyl)Pyridin-2-yl)-N-(6-Methoxy-1-Methyl-1H-Indazol-7-yl)-1H-Pyrazole-4-Sulfonamide (I-115)

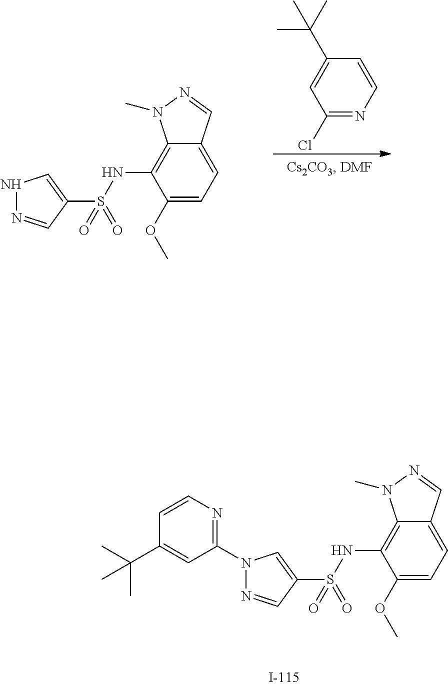 [SMILES: COc1ccc2cnn(C)c2c1NS(=O)(=O)c1cn[nH]c1 ; CC(C)(C)c1ccnc(Cl)c1 ; COc1ccc2cnn(C)c2c1NS(=O)(=O)c1cnn(-c2cc(C(C)(C)C)ccn2)c1]

To a stirred mixture of N-(6-methoxy-1-methylindazol-7-yl)-1H-pyrazole-4-sulfonamide (100 mg, 0.325 mmol, 1 eq.) and Cs₂CO₃ (212.03 mg, 0.650 mmol, 2 eq.) in DMF (4 mL) were added 4-tert-butyl-2-chloropyridine (82.80 mg, 0.488 mmol, 1.5 eq.) at room temperature under a nitrogen atmosphere. The resulting mixture was stirred overnight at 140° C. The resulting mixture was quenched with water (10 mL) and extracted with CH₂Cl₂ (3×10 mL). The combined organic layers were washed with brine (30 mL) and dried over anhydrous Na₂SO₄. After filtration, the filtrate was concentrated under reduced pressure. The residue was purified by prep-HPLC with the following conditions: column: XBridge Prep OBD C18 column, 30*150 mm, 5 m; mobile phase A: water (10 mmol/L NH₄CO₃+0.05% NH₃·H₂O), mobile phase B: ACN; flow rate: 60 mL/min; gradient: 40% B to 65% B in 7 min; wavelength: 254 nm/220 nm; Rt(min): 6.33) to afford 1-(4-(tert-butyl)pyridin-2-yl)-N-(6-methoxy-1-methyl-1H-indazol-7-yl)-1H-pyrazole-4-sulfonamide (8.1 mg, 5.63%) as a white solid. (ES, m/z): [M+H]⁺ 441; ¹H NMR: ¹H NMR (400 MHz, DMSO-d₆) δ 1.34 (s, 9H), 3.35 (s, 3H), 4.24 (s, 3H), 6.88 (d, J=8.8 Hz, 1H), 7.51 (dd, J=5.2, 1.6 Hz, 1H), 7.66 (d, J=8.8 Hz, 1H), 7.93-7.97 (m, 3H), 8.43 (d, J=5.2 Hz, 1H), 8.70 (s, 1H), 9.73 (s, 1H).

### Example 299—Synthesis of 1-(4-(1-Methoxyethyl)Pyridin-2-yl)-N-(1-Methyl-1H-Indazol-7-yl)-1H-Pyrazole-4-Sulfonamide (I-116)

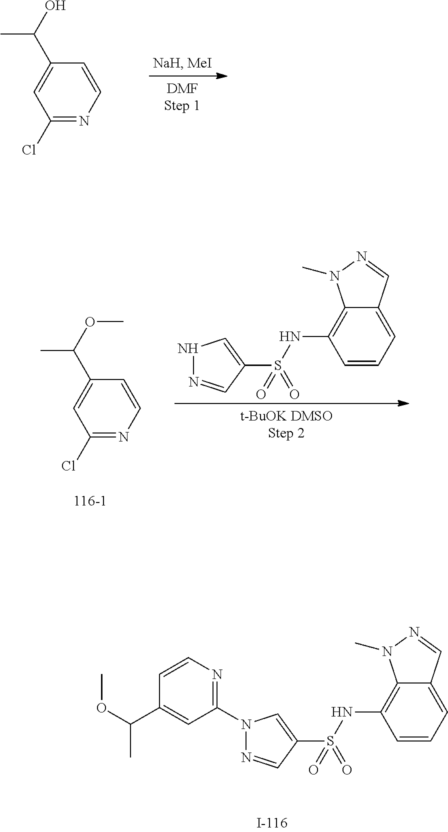

Step 1: Synthesis of 116-1. A solution of 1-(2-chloropyridin-4-yl)ethanol (500 mg, 3.173 mmol, 1 eq.) in DMF (7 mL) was treated with NaH (228.41 mg, 9.519 mmol, 3 eq.) for 30 min at room temperature under a nitrogen atmosphere followed by the addition of Mel (0.40 mL, 6.346 mmol, 2 eq.) dropwise at room temperature. The resulting mixture was stirred for 1 h at room temperature under a nitrogen atmosphere. The resulting mixture was quenched with water (20 ml) and extracted with DCM (3×20 ml). The combined organic layers were dried over anhydrous Na₂SO₄. After filtration, the filtrate was concentrated under reduced pressure. This resulted in 116-1 (200 mg, 36.73%) as a yellow oil.

Step 2: Synthesis of 1-(4-(1-methoxyethyl)pyridin-2-yl)-N-(1-methyl-1H-indazol-7-yl)-1H-pyrazole-4-sulfonamide (I-116). A solution of 116-1 (100 mg, 0.583 mmol, 1 eq.), N-(1-methylindazol-7-yl)-1H-pyrazole-4-sulfonamide (161.58 mg, 0.583 mmol, 1 eq.) and t-BuOK (196.15 mg, 1.749 mmol, 3 eq.) in DMSO (5 mL) were stirred overnight at 140° C. under a nitrogen atmosphere. The residue was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 40 min; detector: UV 254 nm. The resulting mixture was concentrated under reduced pressure. This resulted in 1-(4-(1-methoxyethyl)pyridin-2-yl)-N-(1-methyl-1H-indazol-7-yl)-1H-pyrazole-4-sulfonamide (32.1 mg, 13.09%) as a yellow solid. (ES, m/z): [M+H]⁺ 412; ¹H NMR: ¹H NMR (400 MHz, DMSO-d₆) δ 1.38 (d, J=6.4 Hz, 3H), 3.24 (s, 3H), 4.29 (s, 3H), 4.53 (q, J=6.4 Hz, 1H), 6.72 (d, J=6.8 Hz, 1H), 6.97 (t, J=7.6 Hz, 1H), 7.41 (dd, J=5.2, 0.8 Hz, 1H), 7.66 (d, J=7.6 Hz, 1H), 7.93 (s, 1H), 8.02 (s, 1H), 8.07 (s, 1H), 8.49 (d, J=4.8 Hz, 1H), 8.79 (s, 1H), 10.10 (s, 1H).

### Example 300—Synthesis of N-(1-Methyl-1H-Indazol-7-yl)-1-(5-Methyl-4-(Trifluoromethyl)Pyridin-2-yl)-1H-Pyrazole-4-Sulfonamide (I-117)

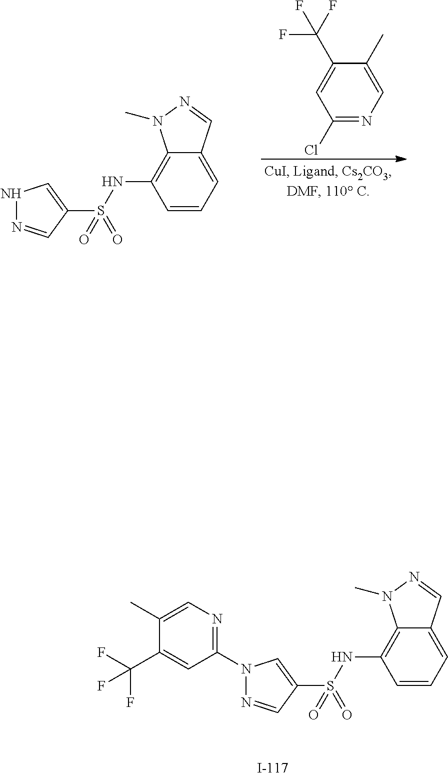 [SMILES: Cn1ncc2cccc(NS(=O)(=O)c3cn[nH]c3)c21 ; Cc1cnc(Cl)cc1C(F)(F)F ; Cc1cnc(-n2cc(S(=O)(=O)Nc3cccc4cnn(C)c34)cn2)cc1C(F)(F)F]

To a stirred solution of N-(1-methylindazol-7-yl)-1H-pyrazole-4-sulfonamide (150 mg, 0.541 mmol, 1 eq.) and 2-chloro-5-methyl-4-(trifluoromethyl)pyridine (211.58 mg, 1.082 mmol, 2 eq.) and Cs₂CO₃ (528.74 mg, 1.623 mmol, 3 eq.) in DMF (3 mL) was added CuI (103.02 mg, 0.541 mmol, 1 eq.) and N1,N2-dimethylcyclohexane-1,2-diamine (76.94 mg, 0.541 mmol, 1 eq.) under a nitrogen atmosphere. The resulting mixture was stirred at 110° C. under a nitrogen atmosphere overnight. The resulting mixture was extracted with EtOAc (3×40 mL). The combined organic layers were dried over anhydrous Na₂SO₄. After filtration, the resulting mixture was concentrated under vacuum. The crude product (55 mg) was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 30% B to 55% B in 7 min; wavelength: 254 nm/220 nm; Rt(min): 6.18) to afford N-(1-methyl-1H-indazol-7-yl)-1-(5-methyl-4-(trifluoromethyl)pyridin-2-yl)-1H-pyrazole-4-sulfonamide (19.0 mg, 8.03%) as a white solid. (ES, m/z): [M+H]⁺ 437; ¹H NMR: 1H NMR (400 MHz, DMSO-d₆) δ 2.51 (s, 3H), 4.29 (s, 3H), 6.72 (d, J=7.2 Hz, 1H), 6.96 (t, J=7.6 Hz, 1H), 7.67 (d, J=8.0 Hz, 1H), 8.07-8.11 (m, 3H), 8.68 (s, 1H), 8.82 (s, 1H), 10.07 (s, 1H).

### Example 301—Synthesis of N-(1-Methyl-1H-Indazol-7-yl)-1-(4-(Tetrahydrofuran-3-yl)Pyridin-2-yl)-1H-Pyrazole-4-Sulfonamide (I-118)

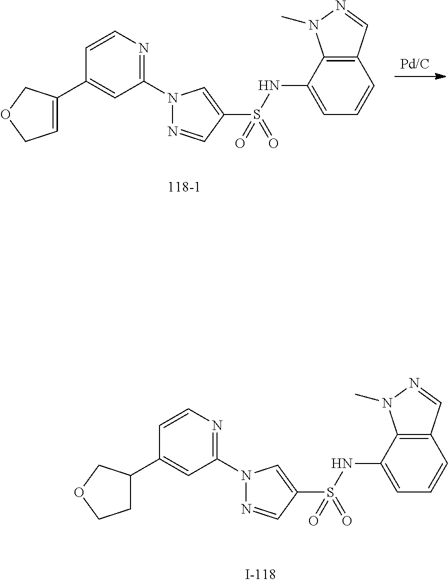 [SMILES: Cn1ncc2cccc(NS(=O)(=O)c3cnn(-c4cc(C5=CCOC5)ccn4)c3)c21 ; Cn1ncc2cccc(NS(=O)(=O)c3cnn(-c4cc(C5CCOC5)ccn4)c3)c21]

A mixture of 118-1 (50 mg, 0.118 mmol, 1 eq.) and Pd/C (10 mg, 0.094 mmol, 0.79 eq.) in EtOAc(10 ml) was stirred for 2 h at room temperature under a hydrogen atmosphere. The resulting mixture was filtered and the filter cake was washed with EtOAc (3×50 mL). The filtrate was concentrated under reduced pressure. The residue was purified by reversed-phase flash chromatography with the following conditions: column: C18 silica gel; mobile phase: MeCN in water (10 mmol/L NH₄CO₃), 30% to 50% gradient in 10 min; detector: UV 254 nm. The crude product (45 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 37% B to 50% B in 7 min; wavelength: 254 nm/220 nm; Rt(min): 6.9) to afford N-(1-methyl-1H-indazol-7-yl)-1-(4-(tetrahydrofuran-3-yl)pyridin-2-yl)-1H-pyrazole-4-sulfonamide (20.8 mg, 40.57%) as a white solid. (ES, m/z): [M+H]⁺ 425; ¹H NMR:(400 MHz, DMSO-d₆) δ 1.90-1.97 (m, 1H), 2.39-2.50 (m, 1H), 3.58-4.07 (m, 5H), 4.28 (s, 3H), 6.70 (d, J=7.2 Hz, 1H), 6.98 (d, J=7.6 Hz, 1H), 7.41 (dd, J=5.2, 1.2 Hz, 1H), 7.70 (d, J=8.0, 1H), 7.89 (s, 1H), 8.02 (s, 1H), 8.09 (s, 1H), 8.43 (d, J=5.2 Hz, 1H), 8.79 (s, 1H), 10.03 (s, 1H).

### Example 302—Synthesis of 1-(4-(3,6-Dihydro-2H-Pyran-4-yl)Pyridin-2-yl)-N-(1-Methyl-1H-Indazol-7-yl)-1H-Pyrazole-4-Sulfonamide (I-119)

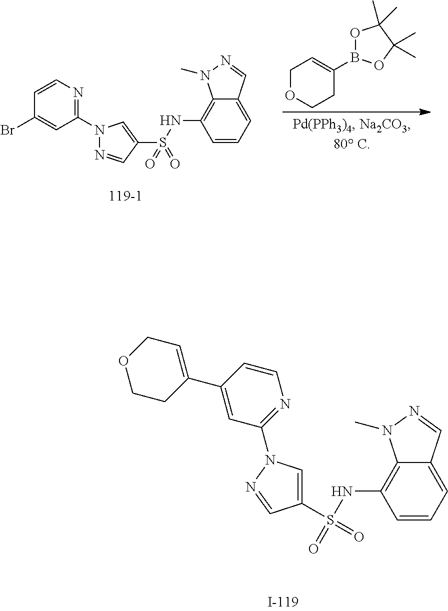 [SMILES: Cn1ncc2cccc(NS(=O)(=O)c3cnn(-c4cc(Br)ccn4)c3)c21 ; CC1(C)OB(C2=CCOCC2)OC1(C)C ; Cn1ncc2cccc(NS(=O)(=O)c3cnn(-c4cc(C5=CCOCC5)ccn4)c3)c21]

To a stirred mixture of 119-1 (100 mg, 0.231 mmol, 1 eq.) 2-(3,6-dihydro-2H-pyran-4-yl)-4,4,5,5-tetramethyl-1,3,2-dioxaborolane (145.46 mg, 0.693 mmol, 3 eq.), Na₂CO₃ (97.85 mg, 0.924 mmol, 4 eq.) and Pd(PPh₃)₄ (26.67 mg, 0.023 mmol, 0.1 eq.) in 1,4-dioxane (10 mL) and H₂O(2 mL) under a nitrogen atmosphere. The resulting mixture was stirred for an additional 4 h at 80° C. The reaction was quenched with water at room temperature. The aqueous layer was extracted with CH₂Cl₂ (3×50 mL). The residue was purified by reversed-phase flash chromatography with the following conditions: column: C18 silica gel; mobile phase: MeCN in water (10 mmol/L NH₄CO₃), 30% to 50% gradient in 10 min; detector: UV 254 nm, to afford 1-(4-(3, 6-dihydro-2H-pyran-4-yl)pyridin-2-yl)-N-(1-methyl-1H-indazol-7-yl)-1H-pyrazole-4-sulfonamide (22.1 mg, 21.45%) as a white solid. (ES, m/z): [M+H]⁺ 437; ¹H NMR:(400 MHz, DMSO-d₆) δ 3.86 (t, J=5.2 Hz, 3H), 4.28-4.30 (m, 6H), 6.71-6.75 (m, 2H), 6.98 (t, J=7.6 Hz, 1H), 7.56 (d, J=5.2, 1.2 Hz, 1H), 7.70 (d, J=8.0 Hz, 1H), 7.95 (s, 1H), 8.01 (s, 1H), 8.08 (s, 1H), 8.47 (d, J=5.2 Hz, 1H), 8.80 (s, 1H), 10.00 (s, 1H).

### Example 303—Synthesis of 1-(5-Fluoro-4-(Trifluoromethyl)Pyridin-2-yl)-N-(1-Methyl-1H-Indazol-7-yl)-1H-Pyrazole-4-Sulfonamide (I-122)

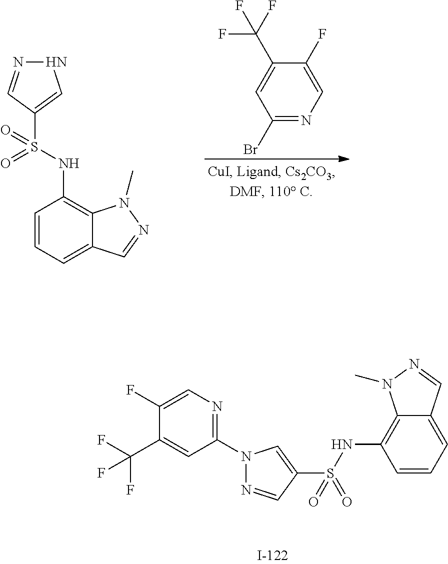 [SMILES: Cn1ncc2cccc(NS(=O)(=O)c3cn[nH]c3)c21 ; Fc1cnc(Br)cc1C(F)(F)F ; Cn1ncc2cccc(NS(=O)(=O)c3cnn(-c4cc(C(F)(F)F)c(F)cn4)c3)c21]

A mixture of N-(1-methylindazol-7-yl)-1H-pyrazole-4-sulfonamide (100 mg, 0.361 mmol, 1 eq.), N1, N2-dimethylcyclohexane-1,2-diamine (51.30 mg, 0.361 mmol, 1 eq.), 2-bromo-5-fluoro-4-(trifluoromethyl)pyridine (87.99 mg, 0.361 mmol, 1 eq.), Cs₂CO₃ (352.49 mg, 1.083 mmol, 3 eq.) and CuI (68.68 mg, 0.361 mmol, 1 eq.) in DMF (2 mL) was stirred overnight at 110° C. under a nitrogen atmosphere. The resulting mixture was diluted with water (20 mL) and extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 1-(5-fluoro-4-(trifluoromethyl)pyridin-2-yl)-N-(1-methyl-1H-indazol-7-yl)-1H-pyrazole-4-sulfonamide (45.6 mg, 27.88%) as a white solid. (ES, m/z): [M+H]⁺ 441; ¹H NMR: (400 MHz, DMSO-d₆) δ 4.29 (s, 3H), 6.73 (d, J=7.2 Hz, 1H), 6.95 (t, J=7.6 Hz, 1H), 7.66 (d, J=8.4 Hz, 1H), 8.07-8.09 (m, 2H), 8.22 (d, J=4.8 Hz, 1H), 8.81 (s, 1H), 8.92 (s, 1H), 10.09 (s, 1H).

### Example 304—Synthesis of N-(1-Methyl-1H-Indazol-7-yl)-1-(4-(Tetrahydro-2H-Pyran-4-yl)Pyridin-2-yl)-1H-Pyrazole-4-Sulfonamide (I-123)

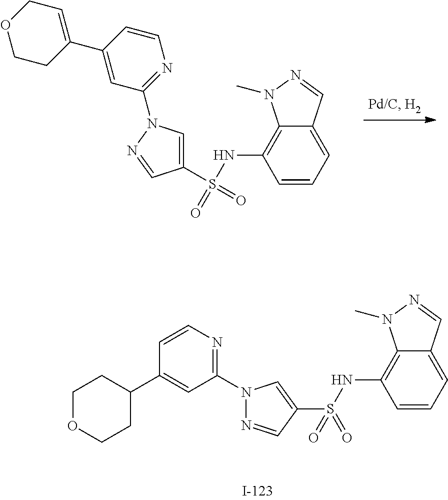 [SMILES: Cn1ncc2cccc(NS(=O)(=O)c3cnn(-c4cc(C5=CCOCC5)ccn4)c3)c21 ; Cn1ncc2cccc(NS(=O)(=O)c3cnn(-c4cc(C5CCOCC5)ccn4)c3)c21]

A mixture of 1-(4-(3,6-dihydro-2H-pyran-4-yl)pyridin-2-yl)-N-(1-methyl-1H-indazol-7-yl)-1H-pyrazole-4-sulfonamide (150 mg, 0.344 mmol, 1 eq.) and Pd/C (50 mg, 0.470 mmol, 1.37 eq.) in EtOAc (10 mL) was stirred overnight at room temperature under a hydrogen atmosphere. The resulting mixture was filtered. The filtrate was concentrated under reduced pressure. The crude product (150 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 37% B to 50% B in 7 min; wavelength: 254 nm/220 nm; Rt(min): 6.9) to afford N-(1-methyl-1H-indazol-7-yl)-1-(4-(tetrahydro-2H-pyran-4-yl)pyridin-2-yl)-1H-pyrazole-4-sulfonamide (13.5 mg, 8.91%) as a white solid. (ES, m/z): [M+H]=439; ¹H NMR:(400 MHz, DMSO-d₆) δ 1.72-1.78 (m, 4H), 2.95-3.03 (m, 1H), 3.46 (t, J=2.4 Hz, 2H), 3.96 (d, J=3.2 Hz, 2H), 4.28 (s, 3H), 6.70 (d, J=6.8 Hz, 1H), 6.97 (d, J=7.6 Hz, 1H), 7.42 (dd, J=5.2, 1.2 Hz, 1H), 7.70 (d, J=7.6 Hz, 1H), 7.87 (d, J=1.2 Hz, 1H), 8.02 (s, 1H), 8.09 (s, 1H), 8.44 (d, J=5.2 Hz, 1H), 8.79 (s, 1H), 10.03 (s, 1H).

### Example 305—Synthesis of 1-(4-(3-Fluorooxetan-3-yl)Pyridin-2-yl)-N-(6-Methoxy-1-Methyl-1H-Pyrazolo[4,3-C]Pyridin-7-yl)-1H-Pyrazole-4-Sulfonamide (I-124)

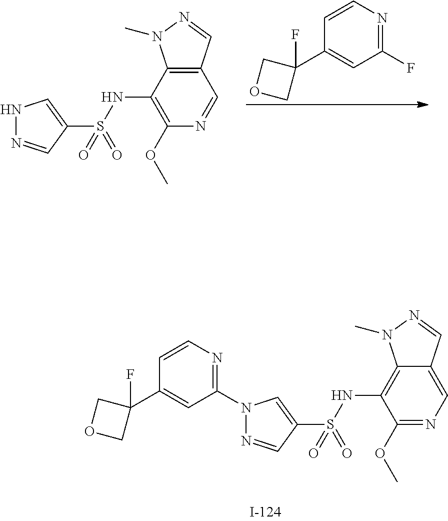

To a stirred solution of N-(6-methoxy-1-methyl-1H-pyrazolo[4,3-c]pyridin-7-yl)-1H-pyrazole-4-sulfonamide (100 mg, 0.324 mmol, 1 eq.) and 2-fluoro-4-(3-fluorooxetan-3-yl)pyridine (61.06 mg, 0.356 mmol, 1.1 eq.) in DMF (2 mL) was added Cs₂CO₃ (317.03 mg, 0.972 mmol, 3 eq.) at room temperature under a nitrogen atmosphere. The resulting mixture was stirred overnight at 140° C. under a nitrogen atmosphere. The resulting mixture was quenched with water (20 mL) and extracted with CH₂Cl₂ (2×20 mL). The combined organic layers were washed with brine (10 mL) and dried over anhydrous Na₂SO₄. After filtration, the filtrate was concentrated under reduced pressure. The crude product (140 mg) was purified by prep-HPLC with the following conditions: column: XBridge Prep OBD C18 column, 30*150 mm, 5 μm; mobile phase A: water (10 mmol/L NH₄CO₃+0.05% NH₃H₂O), mobile phase B: ACN; flow rate: 60 mL/min; gradient: 10% B to 39% B in 7 min; wavelength: 220 nm; Rt(min): 7.88) to afford 1-(4-(3-fluorooxetan-3-yl)pyridin-2-yl)-N-(6-methoxy-1-methyl-1H-pyrazolo[4,3-c]pyridin-7-yl)-1H-pyrazole-4-sulfonamide (51.4 mg, 34.39%) as a white solid. (ES, m/z): [M+H]+ 460; ¹H NMR (400 MHz, DMSO-d₆) δ 3.44 (s, 3H), 4.22 (s, 3H), 4.91-5.08 (m, 4H), 7.70 (d, J=4.8 Hz, 1H), 8.05-8.08 (m, 2H), 8.22 (s, 1H), 8.64 (d, J=3.6 Hz, 2H), 8.78 (s, 1H), 9.88 (s, 1H).

### Example 306—Synthesis of N-(6-Methoxy-1-Methyl-1H-Pyrazolo[4,3-C]Pyridin-7-yl)-1-(4-(3-Methyoxyoxetan-3-yl)Pyridin-2-yl)-1H-Pyrazole-4-Sulfonamide (I-125)

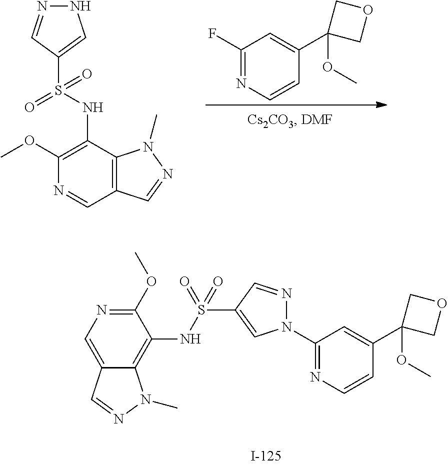 [SMILES: COc1ncc2cnn(C)c2c1NS(=O)(=O)c1cn[nH]c1 ; COC1(c2ccnc(F)c2)COC1 ; COc1ncc2cnn(C)c2c1NS(=O)(=O)c1cnn(-c2cc(C3(OC)COC3)ccn2)c1]

A solution of N-{6-methoxy-1-methylpyrazolo[4,3-c]pyridin-7-yl}-1H-pyrazole-4-sulfonamide (100 mg, 0.324 mmol, 1 eq.), Cs₂CO₃ (211.35 mg, 0.648 mmol, 2 eq.) and 2-fluoro-4-(3-methoxyoxetan-3-yl)pyridine (89.12 mg, 0.486 mmol, 1.5 eq.) in DMF (4 mL) was stirred overnight at 140° C. under a nitrogen atmosphere. This reaction mixture was purified by reversed-phase flash chromatography directly with the following conditions: column: C18 silica gel; mobile phase: MeCN in water (10 mmol/L NH₄CO₃), 0% to 100% gradient in 20 min; detector: UV 254 nm. The crude product (120 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 30% B to 40% B in 7 min; wavelength: 254 nm/220 nm; Rt(min): 6.45) to afford N-{6-methoxy-1-methylpyrazolo [4,3-c]pyridin-7-yl}-1-[4-(3-methoxyoxetan-3-yl)pyridin-2-yl]pyrazole-4-sulfonamide (92.3 mg, 60.24%) as a white solid. (ES, m/z): [M+H]⁺ 472; ¹H NMR: (400 MHz, DMSO-d) δ 3.19 (s, 3H), 3.45 (s, 3H), 4.22 (s, 3H), 4.74 (d, J=7.2 Hz, 2H), 4.88 (d, J=7.6 Hz, 2H), 7.62 (dd, J=5.2, 1.6 Hz, 1H), 8.03 (s, 1H), 8.05 (s, 1H), 8.23 (s, 1H), 8.61 (d, J=5.2 Hz, 1H), 8.66 (s, 1H), 8.78 (s, 1H), 9.95 (s, 1H).

### Example 307—Alternative Synthesis of N-(6-Methoxy-1-Methyl-1H-Pyrazolo[4,3-C]Pyridin-7-yl)-1-(4-(3-Methyoxyoxetan-3-yl)Pyridin-2-yl)-1H-Pyrazole-4-Sulfonamide (I-125)

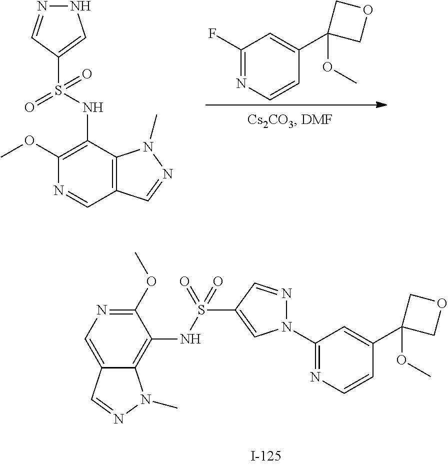 [SMILES: COc1ncc2cnn(C)c2c1NS(=O)(=O)c1cn[nH]c1 ; COC1(c2ccnc(F)c2)COC1 ; COc1ncc2cnn(C)c2c1NS(=O)(=O)c1cnn(-c2cc(C3(OC)COC3)ccn2)c1]

A solution of N-{6-methoxy-1-methylpyrazolo[4,3-c]pyridin-7-yl}-1H-pyrazole-4-sulfonamide (100 mg, 0.324 mmol, 1 eq.), 2-chloro-4-(difluoromethyl)pyridine (79.57 mg, 0.486 mmol, 1.5 eq.) and Cs₂CO₃ (317.03 mg, 0.972 mmol, 3 eq.) in DMF (1.5 mL) was stirred for 16 h at 80° C. under a nitrogen atmosphere. The reaction was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in 1-[4-(difluoromethyl)pyridin-2-yl]-N-{6-methoxy-1-methylpyrazolo[4,3-c]pyridin-7-yl}pyrazole-4-sulfonamide (28.7 mg, 20.16%) as a white solid. (ES, m/z): [M+H]⁺ 436; ¹H NMR: (400 MHz, DMSO-d₆) δ 3.42 (s, 3H), 4.22 (s, 3H), 7.25 (t, J=14.8 Hz, 1H), 7.67 (d, J=5.2 Hz, 1H), 8.08 (s, 1H), 8.13 (s, 1H), 8.23 (s, 1H), 8.67 (s, 1H), 8.71 (d, J=4.8 Hz, 1H), 8.79 (s, 1H), 9.97 (s, 1H).

### Example 308—Synthesis of 1-(4-(Tert-Butyl)Pyridin-2-yl)-N-(6-Methoxy-1-Methyl-1H-Pyrazolo[4,3-C]Pyridin-7-yl)-1H-Pyrazole-4-Sulfonamide (I-127)

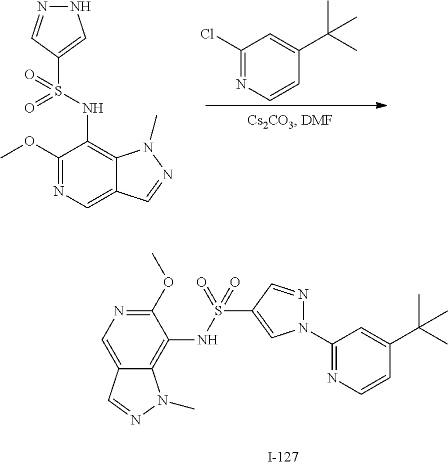 [SMILES: COc1ncc2cnn(C)c2c1NS(=O)(=O)c1cn[nH]c1 ; CC(C)(C)c1ccnc(Cl)c1 ; COc1ncc2cnn(C)c2c1NS(=O)(=O)c1cnn(-c2cc(C(C)(C)C)ccn2)c1]

To a solution of N-{6-methoxy-1-methylpyrazolo[4,3-c]pyridin-7-yl}-1H-pyrazole-4-sulfonamide (100 mg, 0.324 mmol, 1 eq.) and 4-tert-butyl-2-chloropyridine (55.02 mg, 0.324 mmol, 1 eq.) in DMF (1 mL) was added Cs₂CO₃ (317.03 mg, 0.972 mmol, 3 eq.). The resulting mixture was stirred overnight at 140° C. under a nitrogen atmosphere. The resulting mixture was quenched with water (20 mL) and extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. This resulted in 1-(4-(tert-butyl)pyridin-2-yl)-N-(6-methoxy-1-methyl-1H-pyrazolo[4,3-c]pyridin-7-yl)-1H-pyrazole-4-sulfonamide (9.8 mg, 6.80%) as a white solid. (ES, m/z): [M+H]⁺ 442; ¹H NMR: (400 MHz, DMSO-d₆): δ1.34 (s, 9H), 3.45 (s, 3H), 4.22 (s, 3H), 7.52 (d, J=5.2 Hz, 1H), 7.93 (s, 1H), 8.00 (s, 1H), 8.22 (m, 1H), 8.44 (d, J=4.8 Hz, 1H), 8.65 (d, J=1.6 Hz, 1H), 8.73 (s, 1H), 9.92 (s, 1H).

### Example 309—Synthesis of 1-(4-(3-Hydroxytetrahydrofuran-3-yl)Pyridin-2-yl)-N-(1-Methyl-1H-Indazol-7-yl)-1H-Pyrazole-4-Sulfonamide (I-128)

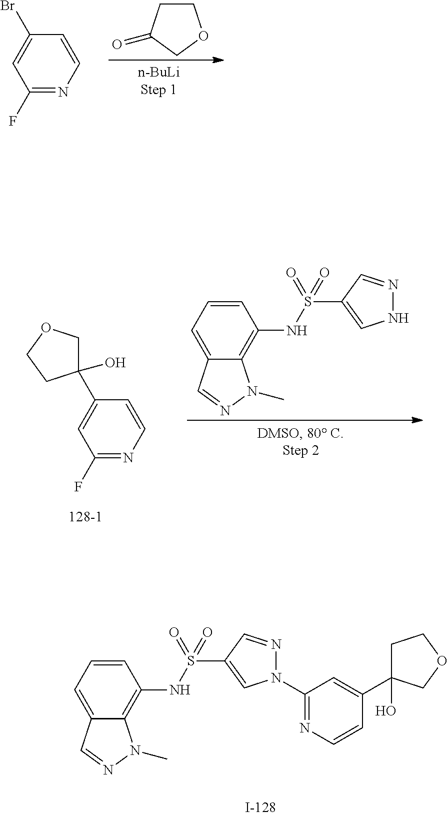

Step 1: Synthesis of 128-1. A solution of 4-bromo-2-fluoropyridine (3 g, 17.047 mmol, 1 eq.) in THE (20 mL) was treated with n-BuLi (3.28 g, 51.141 mmol, 3 eq.) for 1 h at −78° C. under a nitrogen atmosphere followed by the addition of dihydrofuran-3-one (4.40 g, 51.141 mmol, 3 eq.) in portions at −78° C. The resulting mixture was stirred for an additional 2 h at −78° C. to room temperature. The resulting mixture was quenched with water (20 mL). The resulting mixture was extracted with EtOAc (3×30 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in 3-(2-fluoropyridin-4-yl)oxolan-3-ol (2.3 g, 73.66%) as a yellow oil.

Step 2: Synthesis of 1-(4-(3-hydroxytetrahydrofuran-3-yl)pyridin-2-yl)-N-(1-methyl-1H-indazol-7-yl)-1H-pyrazole-4-sulfonamide (I-128). A solution of 3-(2-fluoropyridin-4-yl)oxolan-3-ol (100 mg, 0.546 mmol, 1 eq.), N-(1-methylindazol-7-yl)-1H-pyrazole-4-sulfonamide (120 mg, 0.433 mmol, 0.79 eq.) and Cs₂CO₃ (530 mg, 1.636 mmol, 3.00 eq.) in DMSO (2 mL) was stirred overnight at 80° C. under a nitrogen atmosphere. The resulting mixture was diluted with water (10 mL). The resulting mixture was extracted with EtOAc (3×20 mL). The combined organic layers were washed with brine (30 mL) and dried over anhydrous Na₂SO₄. After filtration, the filtrate was concentrated under reduced pressure. The crude product (60 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 38% B to 50% B in 7 min; wavelength: 254 nm/220 nm; Rt(min): 6.75) to afford 1-(4-(3-hydroxytetrahydrofuran-3-yl)pyridin-2-yl)-N-(1-methyl-1H-indazol-7-yl)-1H-pyrazole-4-sulfonamide (8.1 mg, 3.34%) as an off-white solid. (ES, m/z): [M+H]⁺ 441; ¹H NMR: (400 MHz, DMSO-d₆) δ 2.10-2.13 (m, 1H), 2.29-2.38 (m, 1H), 3.82 (s, 2H), 3.91-4.18 (m, 2H), 4.29 (s, 3H), 5.89 (s, 1H), 6.71 (d, J=7.2 Hz, 1H), 6.96 (t, J=7.6 Hz, 1H), 7.55 (dd, J=5.2, 1.6 Hz, 1H), 7.66 (s, 1H), 8.01 (s, 1H), 8.07 (s, 1H), 8.14 (s, 1H), 8.46 (d, J=5.2 Hz, 1H), 8.79 (s, 1H), 10.02 (s, 1H).

### Example 310—Synthesis of 1-(4-(4-Hydroxytetrahydro-2H-Pyran-4-yl)Pyridin-2-yl)-N-(1-Methyl-1H-Indazol-7-yl)-1H-Pyrazole-4-Sulfonamide (I-129)

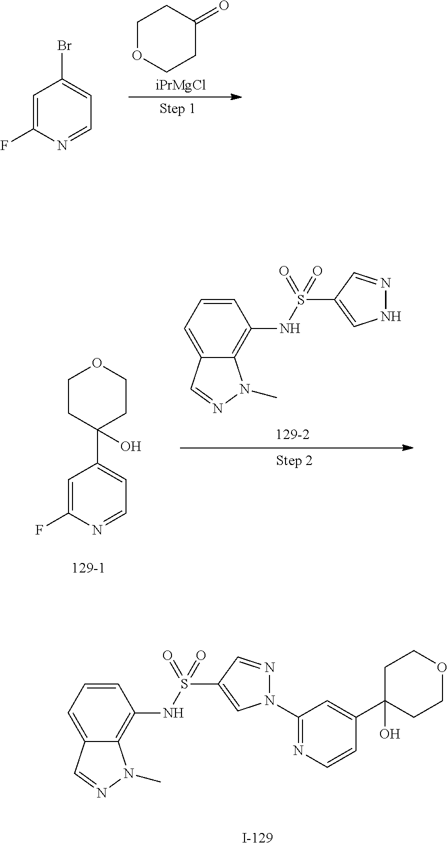 [SMILES: "iPrMgCl" ; Fc1cc(Br)ccn1 ; O=C1CCOCC1 ; OC1(c2ccnc(F)c2)CCOCC1 ; Cn1ncc2cccc(NS(=O)(=O)c3cn[nH]c3)c21 ; Cn1ncc2cccc(NS(=O)(=O)c3cnn(-c4cc(C5(O)CCOCC5)ccn4)c3)c21]

Step 1: Synthesis of 129-1. To a stirred solution of 4-bromo-2-fluoropyridine (2 g, 11.364 mmol, 1 eq.) in THE (100 mL) were added i-PrMgCl LiCl (11.4 mL, 14.773 mmol, 1.3 eq.) dropwise at −78° C. under a nitrogen atmosphere. To the above mixture was added tetrahydro-4H-pyran-4-one (3.41 g, 34.092 mmol, 3 eq.) in portions over 1 h at −78° C. The resulting mixture was stirred for an additional 2 h at −78° C. to room temperature. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. This resulted in 4-(2-fluoropyridin-4-yl)tetrahydro-2H-pyran-4-ol (1.8 g, 80%) as a yellow oil.

Step 2: Synthesis of 1-(4-(4-hydroxytetrahydro-2H-pyran-4-yl)pyridin-2-yl)-N-(1-methyl-1H-indazol-7-yl)-1H-pyrazole-4-sulfonamide (I-129). To a stirred mixture of 4-(2-fluoropyridin-4-yl)tetrahydro-2H-pyran-4-ol (350 mg, 1.775 mmol, 1 eq.) and 129-2 (344.50 mg, 1.242 mmol, 0.7 eq.) in DMSO (1 mL) were added Cs₂CO₃ (1734.76 mg, 5.325 mmol, 3 eq.) in portions at 140° C. under an air atmosphere. The resulting mixture was stirred for 3 h at 140° C. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep Phenyl OBD column, 19*250 mm, 5 μm; mobile phase A: water (10 mmol/L NH₄CO₃+0.05% NH₃H₂O), mobile phase B: MEOH; flow rate: 25 mL/min; gradient: 35% B to 55% B in 10 min; wavelength: 254 nm/220 nm; Rt(min): 9.77). This resulted in 1-(4-(4-hydroxytetrahydro-2H-pyran-4-yl)pyridin-2-yl)-N-(1-methyl-1H-indazol-7-yl)-1H-pyrazole-4-sulfonamide (13.2 mg, 1.61%) as a white solid. (ES, m/z): [M+H]⁺=455; ¹H NMR: 1H NMR (400 MHz, DMSO-d₆) δ 1.54 (d, J=12.8 Hz, 2H), 1.98-2.08 (m, 2H), 3.42-3.82 (m, 4H), 4.31 (s, 3H), 5.54 (s, 1H), 6.75 (d, J=7.2 Hz, 1H), 6.91 (t, J=7.6 Hz, 1H), 7.50-7.54 (m, 2H), 7.99 (s, 1H), 8.01 (s, 1H), 8.11 (s, 1H), 8.45 (d, J=5.2 Hz, 1H), 8.75 (s, 1H), 9.97 (s, 1H).

### Example 311—Synthesis of 1-(4-(4-Fluorotetrahydro-2H-Pyran-4-yl)Pyridin-2-yl)-N-(1-Methyl-1H-Indazol-7-yl)-1H-Pyrazole-4-Sulfonamide (I-130)

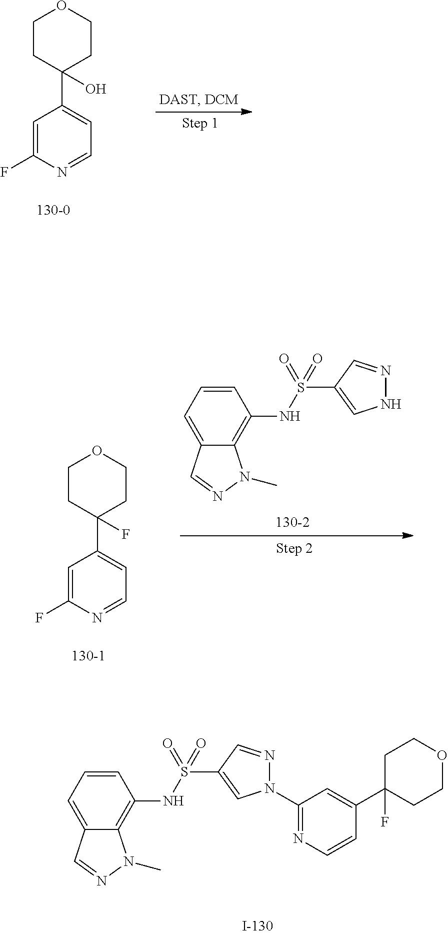

Step 1: Synthesis of 130-1. To a stirred solution of 130-0 (600 mg, 3.042 mmol, 1 eq.) in DCM (10 mL) was added DAST (980.83 mg, 6.084 mmol, 2 eq.) dropwise at 0° C. under an air atmosphere. The resulting mixture was stirred for 2 h at room temperature. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. The residue was purified by silica gel column chromatography and eluted with PE/EA (5:1) to afford 130-1 (120 mg, 19.80%) as a yellow solid.

Step 2: Synthesis of 1-(4-(4-fluorotetrahydro-2H-pyran-4-yl)pyridin-2-yl)-N-(1-methyl-1H-indazol-7-yl)-1H-pyrazole-4-sulfonamide (I-130). To a stirred mixture of 130-1 (150 mg, 0.753 mmol, 1 eq.) and 130-2 (146.17 mg, 0.527 mmol, 0.7 eq.) in DMSO (4 mL) were added Cs₂CO₃ (736.03 mg, 2.259 mmol, 3 eq.) in portions at 80° C. under a nitrogen atmosphere. The resulting mixture was stirred for 3 h at 80° C. The resulting mixture was quenched with water (50 mL) and extracted with EtOAc (5×20 mL). The combined organic layers were washed with brine (10 mL) and dried over anhydrous Na₂SO₄. After filtration, the filtrate was concentrated under reduced pressure. The crude product (150 mg) was purified by prep-HPLC with the following conditions: column: Xselect CSH C18 OBD column 30*150 mm 5 μm; mobile phase A: water (0.1% FA), mobile phase B: ACN; flow rate: 60 mL/min; gradient: 38% B to 50% B in 7 min; wavelength: 254 nm/220 nm; Rt(min): 6.75) to afford 1-(4-(4-fluorotetrahydro-2H-pyran-4-yl)pyridin-2-yl)-N-(1-methyl-1H-indazol-7-yl)-1H-pyrazole-4-sulfonamide (9.6 mg, 2.75%) as a white solid. (ES, m/z): [M+H]⁺=457; ¹H NMR: 1H NMR (400 MHz, DMSO-d₆) δ 1.87-1.91 (m, 2H), 2.15-2.33 (m, 2H), 3.68-3.74 (m, 2H), 3.88-3.92 (m, 2H), 4.29 (s, 3H), 6.73 (d, J=7.2, 1H), 6.96 (t, J=7.6 Hz, 1H), 7.57 (d, J=1.2 Hz, 1H), 7.65 (d, J=8.0 Hz, 1H), 8.00 (s, 1H), 8.03 (s, 1H), 8.07 (s, 1H), 8.55 (d, J=5.2 Hz, 1H), 8.81 (s, 1H), 10.16 (s, 1H).

### Example 312—Synthesis of 1-(4-(4-Methoxytetrahydro-2H-Pyran-4-yl)Pyridin-2-yl)-N-(1-Methyl-1H-Indazol-7-yl)-1H-Pyrazole-4-Sulfonamide (I-131)

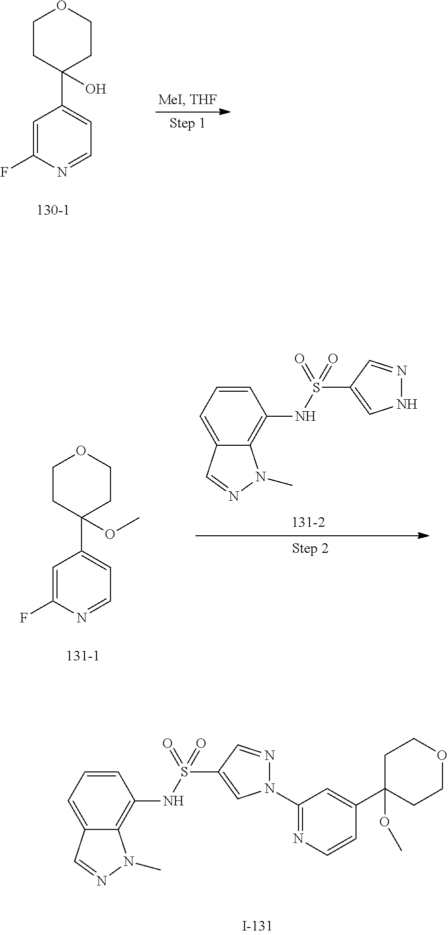

Step 1: Synthesis of 131-1. To a stirred solution of 131-0 (600 mg, 3.042 mmol, 1 eq.) and NaH (219.04 mg, 9.126 mmol, 3 eq.) in THE (10 mL) was added Mel (1295.53 mg, 9.126 mmol, 3 eq.) dropwise at 0° C. under an air atmosphere. The resulting mixture was stirred for an additional 3 h at room temperature. The reaction was quenched with MeOH at room temperature. The aqueous layer was extracted with EtOAc (5×1 mL). The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in 131-1 (160 mg, 24.90%) as a light yellow oil.

Step 2: Synthesis of 1-(4-(4-methoxytetrahydro-2H-pyran-4-yl)pyridin-2-yl)-N-(1-methyl-1H-indazol-7-yl)-1H-pyrazole-4-sulfonamide (I-131). To a stirred mixture of 131-1 (150 mg, 0.710 mmol, 1 eq.) and 131-2 (137.84 mg, 0.497 mmol, 0.7 eq.) in DMSO (4 mL) were added Cs₂CO₃(694.10 mg, 2.130 mmol, 3 eq.) in portions at 140° C. under an air atmosphere. The resulting mixture was stirred overnight at 140° C. The resulting mixture was extracted with EtOAc (5 xl mL). The combined organic layers were washed with water (1 mL) and dried over anhydrous Na₂SO₄. After filtration, the filtrate was concentrated under reduced pressure. The crude product (150 mg) was purified by prep-HPLC with the following conditions: column: YMC-Actus Triant C18 ExRs column, 20*250 mm, 5 μm; mobile phase A: water (10 mmol/L NH₄CO₃+0.05% NH₃H₂O), mobile phase B: ACN; flow rate: 25 mL/min; gradient: 27% B to 28% B in 12 min; wavelength: 220 nm; Rt(min): 11.28) to afford the title compound (19.3 mg, 5.66%) as a white solid. (ES, m/z): [M+H]⁺ 469; ¹H NMR: 1H NMR (400 MHz, DMSO-d₆) δ 1.91-1.99 (m, 4H), 3.00 (s, 3H), 2.66-3.77 (m, 4H), 4.30 (s, 3H), 6.74 (d, J=7.2 Hz, 1H), 6.96 (t, J=8.0 Hz, 1H), 7.50 (d, J=5.2 Hz, 1H), 7.64 (s, 1H), 7.93 (s, 1H), 8.02 (s, 1H), 8.06 (s, 1H), 8.53 (d, J=5.2 Hz, 1H), 8.80 (s, 1H), 10.04 (s, 1H).

### Example 313—Synthesis of N-(6-Methoxy-1-Methyl-1H-Indazol-7-yl)-1-(4-(1-Methoxycyclobutyl)Pyridin-2-yl)-1H-Pyrazole-4-Sulfonamide (I-132)

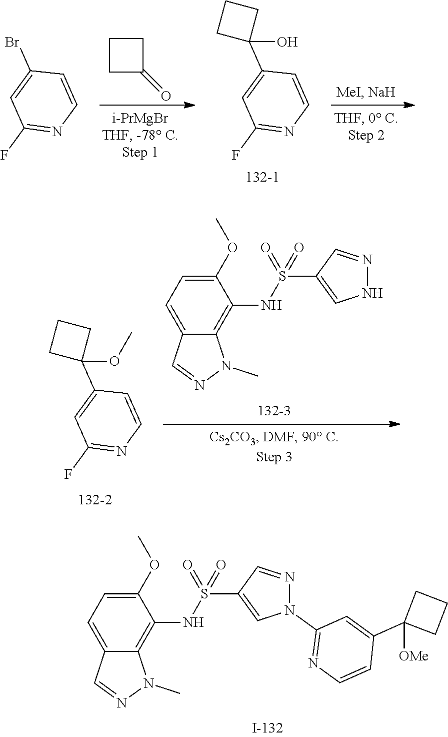

Step 1: Synthesis of 132-1. A solution of 4-bromo-2-fluoropyridine (2 g, 11.364 mmol, 1 eq.) in THF (20 mL) was treated with the solution of i-PrMgBr LiCl in THF (13.1 mL, 1.3 M, 1.5 eq.) for 30 min at −78° C. under a nitrogen atmosphere followed by the addition of cyclobutanone (1.59 g, 22.728 mmol, 2 eq.) dropwise at −78° C. The resulting mixture was stirred for 2 h at −78° C. under a nitrogen atmosphere. The reaction was quenched with sat. NH₄Cl (aq. 10 mL) at 0° C. The resulting mixture was extracted with CH₂Cl₂ (3×30 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 40% to 43% gradient in 20 min; detector: UV 215 nm. This resulted in 132-1 (0.6 g, 31.58%) as a light yellow oil.

Step 2: Synthesis of 132-2. A solution of 131-1 (600 mg, 3.589 mmol, 1 eq.) in THE (10 mL) was treated with NaH (175 mg, 4.375 mmol, 1.22 eq., 60%) for 30 min at 0° C. under a nitrogen atmosphere followed by the addition of Mel (340 uL, 5.462 mmol, 1.52 eq.) dropwise at 0° C. The resulting mixture was stirred for 2 h at room temperature under a nitrogen atmosphere. The reaction was quenched by the addition of sat. NH₄Cl (aq.) (10 mL) at 0° C. The resulting mixture was extracted with CH₂Cl₂ (3×10 mL). The combined organic layers were washed with brine (5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 58% to 60% gradient in 10 min; detector: UV 254 nm. This resulted in 132-2 (340 mg, 52.28%) as a light yellow liquid.

Step 3: Synthesis of N-(6-methoxy-1-methyl-1H-indazol-7-yl)-1-(4-(1-methoxy-cyclobutyl)pyridin-2-yl)-1H-pyrazole-4-sulfonamide (I-132). To a stirred solution of 132-3 (150 mg, 0.488 mmol, 1.00 eq.) and Cs₂CO₃ (477 mg, 1.464 mmol, 3.00 eq.) in DMF (8 mL) was added 132-2 (173.3 mg, 0.956 mmol, 1.96 eq.) dropwise at room temperature under a nitrogen atmosphere. The resulting mixture was stirred for 2 h at 90° C. under a nitrogen atmosphere. The resulting mixture was filtered and the filter cake was washed with DMF (3×8 mL). The filtrate was concentrated under reduced pressure. The residue was purified by reversed-phase flash chromatography with the following conditions: column: C18 silica gel; mobile phase: MeCN in water (0.1% TFA), 48% to 50% gradient in 20 min; detector: UV 215 nm. This resulted in N-(6-methoxy-1-methyl-1H-indazol-7-yl)-1-(4-(1-methoxycyclobutyl) pyridin-2-yl)-1H-pyrazole-4-sulfonamide (67.6 mg, 29.38%) as a light brown solid. (ES, m/z): [M+H]⁺ 469; ¹H NMR: (400 MHz, DMSO-d₆) δ 1.72-1.79 (m, 1H), 1.90-1.97 (m, 1H), 2.32-2.39 (m, 4H), 2.97 (s, 3H), 3.35 (s, 3H), 4.25 (s, 3H), 6.88 (d, J=9.2 Hz, 1H), 7.52 (dd, J=9.2, 1.2 Hz, 1H), 7.68 (d, J=8.8 Hz, 1H), 7.94 (s, 1H), 7.99 (s, 1H), 8.01 (s, 1H), 8.54 (d, J=5.2 Hz, 1H), 8.73 (s, 1H), 9.80 (s, 1H).

### Example 314—Synthesis of 1-(4-(Difluoromethyl)Pyridin-2-yl)-N-(6-Methoxy-1-Methyl-1H-Indazol-7-yl)-1H-Pyrazole-4-Sulfonamide (I-133)

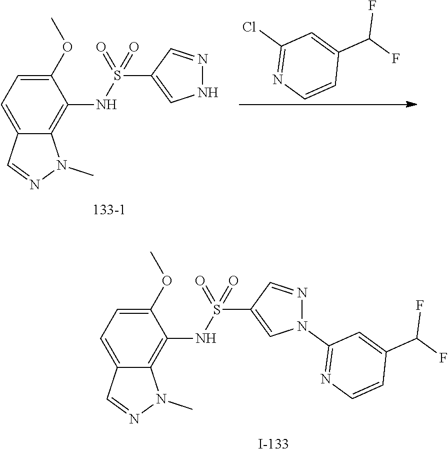 [SMILES: COc1ccc2cnn(C)c2c1NS(=O)(=O)c1cn[nH]c1 ; FC(F)c1ccnc(Cl)c1 ; COc1ccc2cnn(C)c2c1NS(=O)(=O)c1cnn(-c2cc(C(F)F)ccn2)c1]

A mixture of 133-1 (100 mg, 0.325 mmol, 1 eq.), Ephos (34.80 mg, 0.065 mmol, 0.2 eq.), 2-chloro-4-(difluoromethyl)pyridine (63.86 mg, 0.390 mmol, 1.2 eq.) and Ephos Pd G4 (29.89 mg, 0.033 mmol, 0.1 eq.) in 1,4-dioxane (15 mL) was stirred for 3 h at 90° C. under a nitrogen atmosphere. The resulting mixture was concentrated under reduced pressure. The reaction was quenched with water at room temperature. The aqueous layer was extracted with CH₂Cl₂ (3×100 mL). The residue was purified by reversed-phase flash chromatography with the following conditions: column: C18 silica gel; mobile phase: MeCN in water (10 mmol/L NH₄CO₃), 30% to 50% gradient in 15 min; detector: UV 254 nm. This resulted in 1-(4-(difluoromethyl)pyridine-2-yl)-N-(6-methoxy-1-methyl-1H-indazol-7-yl)-1H-pyrazole-4-sulfonamide (84.9 mg, 59.64%) as an off-white solid. (ES, m/z): [M+H]⁺ 435; ¹H NMR:(400 MHz, DMSO-d₆) δ 3.33 (s, 3H), 4.25 (s, 3H), 6.88 (d, J=8.8 Hz, 1H), 7.25 (t, J=14.8 Hz, 1H), 7.66-7.69 (m, 2H), 7.99 (s, 1H), 8.06 (s, 1H), 8.13 (s, 1H), 8.71 (d, J=5.2 Hz, 1H), 8.75 (s, 1H), 9.85 (s, 1H).

### Example 315—Synthesis of 1-(4-(1-Hydroxyethyl)Pyridin-2-yl)-N-(6-Methoxy-1-Methyl-1H-Indazol-7-yl)-1H-Pyrazole-4-Sulfonamide (I-134)

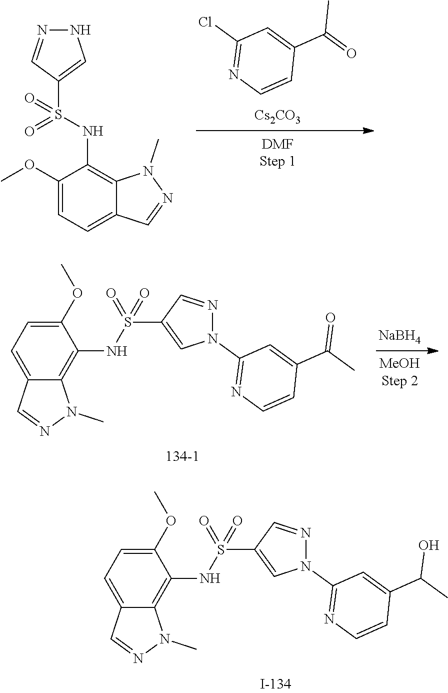

Step 1: Synthesis of 134-1. To a stirred mixture of N-(6-methoxy-1-methyl-1H-indazol-7-yl)-1H-pyrazole-4-sulfonamide (150 mg, 0.488 mmol, 1.00 eq.), 1-(2-chloropyridin-4-yl)ethan-1-one (91 mg, 0.585 mmol, 1.20 eq.) and Cs₂CO₃ (477 mg, 1.464 mmol, 3.00 eq.) in DMF (5 mL) were added (1R,2R)—N1,N2-dimethylcyclohexane-1,2-diamine (139 mg, 0.977 mmol, 2.00 eq.) and CuI (19 mg, 0.100 mmol, 0.20 eq.) at room temperature under a nitrogen atmosphere. The resulting mixture was stirred overnight at 100° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The residue was purified by silica gel column chromatography and eluted with PE/EA (5:1) to afford 1-(4-acetylpyridin-2-yl)-N-(6-methoxy-1-methyl-1H-indazol-7-yl)-1H-pyrazole-4-sulfonamide (83 mg, 39.88%) as a yellow solid.

Step 2: Synthesis of 1-(4-(1-hydroxyethyl)pyridin-2-yl)-N-(6-methoxy-1-methyl-1H-indazol-7-yl)-1H-pyrazole-4-sulfonamide (I-134). To a stirred solution of 1-(4-acetylpyridin-2-yl)-N-(6-methoxy-1-methyl-1H-indazol-7-yl)-1H-pyrazole-4-sulfonamide (80 mg, 0.188 mmol, 1.00 eq.) in MeOH (5 mL) were added NaBH₄ (14.1 mg, 0.376 mmol, 2.00 eq.) at 0° C. under a nitrogen atmosphere. The resulting mixture was stirred for 2 h at room temperature under a nitrogen atmosphere. The reaction was quenched by the addition of sat. NH4Cl (aq.) (20 mL) at 0° C. The resulting mixture was extracted with EtOAc (3×20 mL). The combined organic layers were washed with brine (60 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 40% B to 60% B in 7 min; wavelength: 254 nm/220 nm; Rt(min): 7.08 to afford 1-(4-(1-hydroxyethyl)pyridin-2-yl)-N-(6-methoxy-1-methyl-1H-indazol-7-yl)-1H-pyrazole-4-sulfonamide (15.9 mg, 19.78%) as a white solid. (ES, m/z): [M+H]⁺ 429; ¹H NMR: (400 MHz, DMSO-d₆) δ 1.37 (d, J=6.4 Hz, 3H), 3.37 (s, 3H), 4.34 (s, 3H), 4.82-4.85 (m, 1H), 5.54 (d, J=4.4 Hz, 1H), 6.67 (d, J=8.4 Hz, 1H), 7.04 (d, J=8.4 Hz, 1H), 7.29 (d, J=4.8 Hz, 1H), 7.73 (d, J=9.2 Hz, 2H), 7.94 (s, 1H), 8.37 (d, J=5.2 Hz, 1H), 8.44 (s, 1H).

### Example 316—Synthesis of 1-(4-(2-Hydroxypropan-2-yl)Pyridin-2-yl)-N-(6-Methoxy-1-Methyl-1H-Indazol-7-yl)-1H-Pyrazole-4-Sulfonamide (I-135)

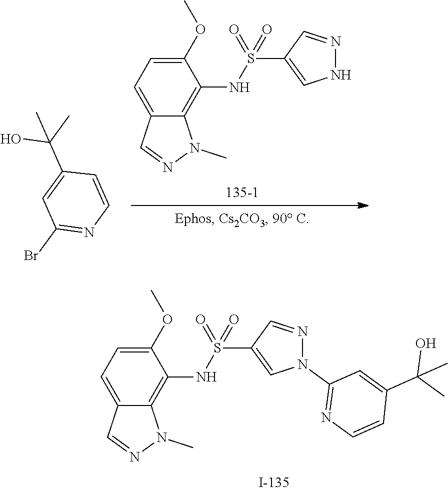 [SMILES: CC(C)(O)c1ccnc(Br)c1 ; COc1ccc2cnn(C)c2c1NS(=O)(=O)c1cn[nH]c1 ; COc1ccc2cnn(C)c2c1NS(=O)(=O)c1cnn(-c2cc(C(C)(C)O)ccn2)c1]

A solution of 135-1 (100 mg, 0.325 mmol, 1 eq.) in 1,4-dioxane was treated with 2-(2-bromopyridin-4-yl)propan-2-ol (84.37 mg, 0.390 mmol, 1.2 eq.) for 2 min at room temperature under a nitrogen atmosphere followed by the addition of EPhos (34.80 mg, 0.065 mmol, 0.2 eq.), EPhos Pd G4 (59.71 mg, 0.065 mmol, 0.2 eq.) and Cs₂CO₃ (318.05 mg, 0.975 mmol, 3 eq.) in portions at 90° C. The resulting mixture was stirred for an additional 2 h at 90° C. under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in 1-(4-(2-hydroxypropan-2-yl)pyridin-2-yl)-N-(6-methoxy-1-methyl-1H-indazol-7-yl)-1H-pyrazole-4-sulfonamide (34.7 mg, 24.00%) as a white solid. (ES, m/z): [M+H]⁺ 443; ¹H NMR: (400 MHz, DMSO-d₆) δ 1.47 (s, 6H), 3.34 (s, 3H), 4.25 (s, 3H), 6.88 (d, J=8.8 Hz, 1H) 7.51 (dd, J=5.2, 1.2 Hz, 1H), 7.68 (d, J=8.8 Hz, 1H), 7.98 (s, 1H), 7.99 (s, 1H), 8.08 (s, 1H), 8.43 (d, J=5.2 Hz, 1H), 8.71 (s, 1H).

### Example 317—Synthesis of N-(6-Fluoro-1-Methyl-1H-Indazol-7-yl)-1-(4-(Trifluoromethyl)Pyridin-2-yl)-1H-Pyrazole-4-Sulfonamide (I-136)

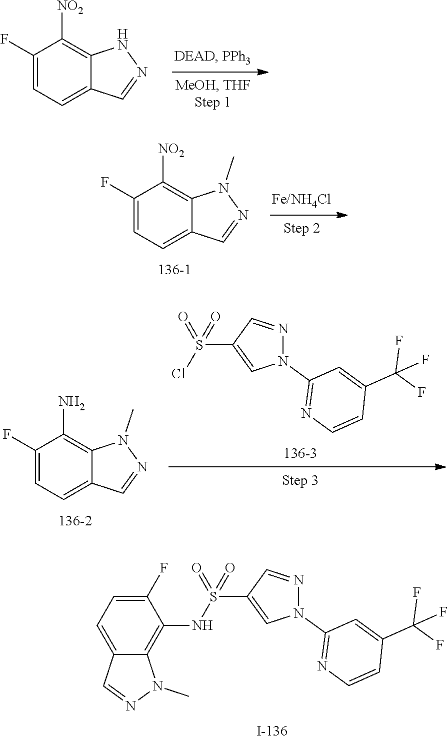 [SMILES: O=[N+]([O-])c1c(F)ccc2cn[nH]c12 ; Cn1ncc2ccc(F)c([N+](=O)[O-])c21 ; Cn1ncc2ccc(F)c(N)c21 ; O=S(=O)(Cl)c1cnn(-c2cc(C(F)(F)F)ccn2)c1 ; Cn1ncc2ccc(F)c(NS(=O)(=O)c3cnn(-c4cc(C(F)(F)F)ccn4)c3)c21]

Step 1: Synthesis of 136-1. A solution of 6-fluoro-7-nitro-1H-indazole (320 mg, 1.767 mmol, 1 eq.), MeOH (566.09 mg, 17.670 mmol, 10 eq.), PPh₃ (695.10 mg, 2.651 mmol, 1.5 eq.) and DIAD (535.87 mg, 2.651 mmol, 1.5 eq.) in THE (6 mL) was stirred overnight from 0° C. to room temperature. After the reaction was completed, the reaction was quenched by the addition of sat. NH₄Cl (aq.) (50 mL), the resulting mixture was extracted with EtOAc (4×50 mL). The combined organic layers were washed with the brine (50 mL) and dried over anhydrous Na₂SO₄. After filtration, the filtrate was concentrated under reduced pressure to give a mixture. The mixture was purified by reversed-phase flash chromatography with the following conditions: column C₁₈ silica gel; mobile phase: ACN in water (10 mmol/L NH₄CO₃), 0 to 100% gradient in 25 min; detector: UV 254 nm, to give 136-1 (840 mg) as a yellow solid.

Step 2: Synthesis of 136-2. A solution of 136-1 (840 mg, 4.30 mmol, 1 eq.), Fe (2403.74 mg, 43.040 mmol, 10 eq.) and NH₄Cl (2302.38 mg, 43.04 mmol, 10 eq.) in MeOH (40 mL) was stirred overnight at 50° C. After the reaction was completed, the solid was filtrated and washed with MeOH (40 mL). The solution was combined and concentrated under vacuum to give the crude product. The residue purified by reversed-phase flash chromatography with the following conditions: column: C18 silica gel; mobile phase: ACN in water (10 mmol/L NH₄CO₃), 0% to 100% gradient in 30 min; detector: UV 254 nm, to give 136-2 (53 mg, 7.45%) yellow solid as product.

Step 3: Synthesis of N-(6-fluoro-1-methyl-1H-indazol-7-yl)-1-(4-(trifluoromethyl) pyridin-2-yl)-1H-pyrazole-4-sulfonamide (I-136). A solution of 136-2 (181.14 mg, 0.58 mmol, 2 eq.), 136-3 and DMAP (7.10 mg, 0.06 mmol, 0.2 eq.) in pyridine (3 mL) was stirred overnight at 80° C. After the reaction was completed, the solvent was removed under vacuum to afford the crude product. The residue was purified by reversed-phase flash chromatography with the following conditions: column: C18 silica gel; mobile phase: ACN in water (10 mmol/L NH₄CO₃), 0% to 100% gradient in 30 min; detector: UV 254 nm, to give the crude product. The crude product was purified by prep-HPLC with the following conditions: column: Xselect CSH C₁₈ OBD column 30*150 mm 5 μm; mobile phase A: water (0.1% FA), mobile phase B: ACN; flow rate: 60 mL/min; gradient: 47% B to 60% B in 7 min; wavelength: 254 nm,/220 nm; Rt(min): 6.2) to afford N-(6-fluoro-1-methyl-1H-indazol-7-yl)-1-(4-(trifluoromethyl)pyridin-2-yl)-1H-pyrazole-4-sulfonamide (18.2 mg, 14.21%) as a white solid. (ES, m/z): [M+H]⁺ 441; ¹H NMR: ¹H NMR (400 MHz, DMSO-d) δ 4.27 (s, 3H), 6.93 (dd, J=10.4, 8.8 Hz, 1H), 7.67 (dd, J=8.8, 4.8 Hz, 1H), 7.87 (d, J=4.4 Hz, 1H), 8.08 (s, 1H), 8.11 (s, 1H), 8.16 (s, 1H), 8.81-8.84 (m, 2H), 10.70 (s, 1H).

### Example 318—Synthesis of 5-Methyl-N-(1-Methyl-1H-Indazol-7-yl)-1-(4-(Trifluoromethyl)Pyridin-2-yl)-1H-Pyrazole-4-Sulfonamide (I-137)

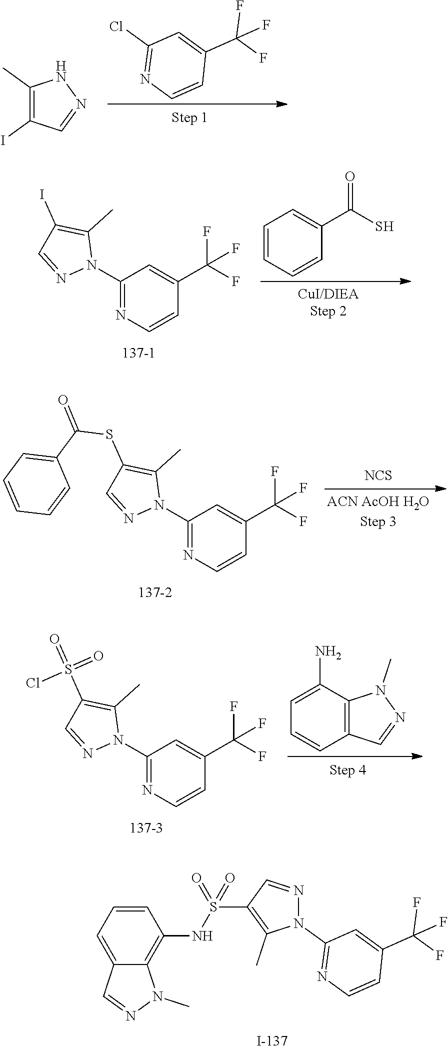

Step 1: Synthesis of 137-1. A solution of 4-iodo-3-methyl-2H-pyrazole (5 g, 24.038 mmol, 1.1 eq.), 2-chloro-4-(trifluoromethyl)pyridine (3.97 g, 21.853 mmol, 1 eq.) and t-BuOK (7.36 g, 65.558 mmol, 3 eq.) in ACN (15 mL) was stirred overnight at 80° C. The resulting mixture was quenched with H₂O (50 mL) and extracted with DCM (4×50 ml). The combined organic layers were dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 60 min; detector: UV 254 nm. This resulted in 137-1 (2.6 g, 33.70%) as a dark yellow solid.

Step 2: Synthesis of 137-2. A solution of 137-1 (2.6 g, 7.364 mmol, 1 eq.), benzenecarbothioic acid (1.22 g, 8.837 mmol, 1.2 eq.), 1,10-phenanthroline (0.27 g, 1.473 mmol, 0.2 eq.), DIEA (1.90 g, 14.728 mmol, 2 eq.) and CuI (0.14 g, 0.736 mmol, 0.1 eq.) in toluene (30 mL) was stirred for 8 hours at 110° C. under a nitrogen atmosphere. The resulting mixture was concentrated under reduced pressure. The residue was dissolved in water (30 mL) and extracted with DCM (3×30 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 40 min; detector: UV 254 nm. The resulting mixture was concentrated under reduced pressure. This resulted in 137-2 (190 mg, 7.10%) as a dark yellow solid.

Step 3: Synthesis of 137-3. A solution of 137-2 (140 mg, 0.385 mmol, 1 eq.) and NCS (154.34 mg, 1.155 mmol, 3 eq.) in MeCN (5 mL), AcOH (0.5 mL) and H₂O (0.5 mL) was stirred for 3 hours at room temperature. The resulting mixture was quenched with H₂O (20 mL) and extracted with DCM (3×20 ml). The combined organic layers were washed with brine (30 mL) and dried over anhydrous Na₂SO₄. After filtration, the filtrate was concentrated under reduced pressure. This resulted in 137-3 (190 mg, crude) as a yellow oil.

Step 4: Synthesis of 5-methyl-N-(1-methyl-1H-indazol-7-yl)-1-(4-(trifluoromethyl) pyridin-2-yl)-1H-pyrazole-4-sulfonamide (I-137). A solution of 137-3 (160 mg, 0.491 mmol, 1 eq.) and 1-methylindazol-7-amine (72.30 mg, 0.491 mmol, 1 eq.) in pyridine (5 mL) was stirred for 6 hours at room temperature. The resulting mixture was concentrated under reduced pressure. The residue was dissolved in water (10 mL) and extracted with EtOAc (3×15 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 40 min; detector: UV 254 nm. The resulting mixture was concentrated under reduced pressure. This resulted in 5-methyl-N-(1-methyl-1H-indazol-7-yl)-1-(4-(trifluoromethyl)pyridin-2-yl)-1H-pyrazole-4-sulfonamide (26.6 mg, 12.41%) as a yellow solid. (ES, m/z): [M+H]⁺ 436; ¹H NMR (400 MHz, DMSO-d₆) δ 2.42 (s, 3H), 4.28 (s, 3H), 6.70 (d, J=7.2 Hz, 1H), 6.99 (t, J=7.6 Hz, 1H), 7.73 (d, J=7.6 Hz, 1H), 7.91 (d, J=5.2 Hz, 1H), 7.94 (s, 1H), 8.10 (s, 1H), 8.16 (s, 1H), 8.84 (d, J=5.2 Hz, 1H), 10.02 (s, 1H).

### Example 319—Synthesis of 1-(4-(3,3-Difluoro-1-Methoxycyclobutyl) Pyridin-2-yl)-N-(1-Methyl-1H-Indazol-7-yl)-1H-Pyrazole-4-Sulfonamide (I-138)

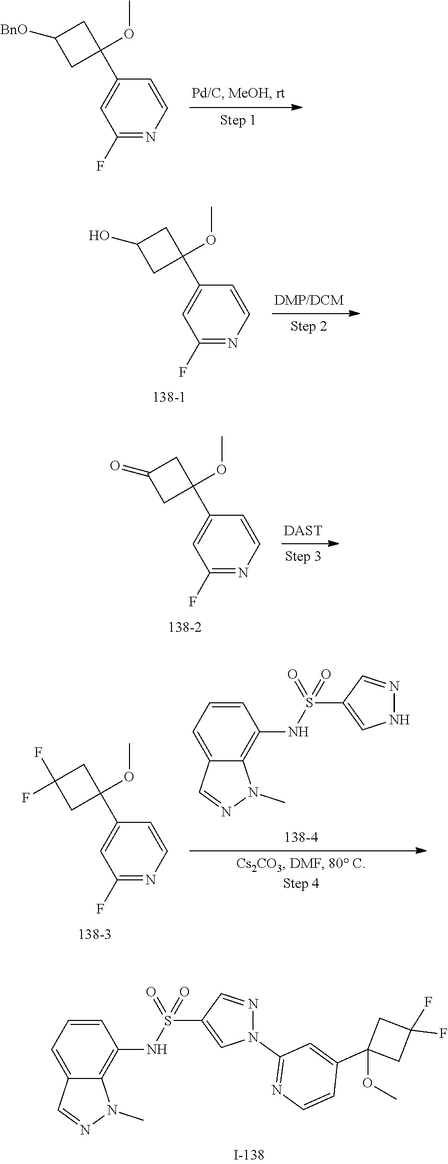

Step 1: Synthesis of 138-1. To a solution of 4-[3-(benzyloxy)-1-methoxycyclobutyl]-2-fluoropyridine (870 mg, 3.028 mmol, 1 eq.) in 10 mL MeOH was added Pd/C (10%, 0.033 g) under a nitrogen atmosphere. The mixture was hydrogenated at room temperature for 2 h under a hydrogen atmosphere. The resulting mixture was filtered and the filter cake was washed with MeOH (3×50 mL). The filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 138-1 (280 mg, 51%) as a yellow oil.

Step 2: Synthesis of 138-2. To a stirred solution of 138-1 (280 mg, 1.420 mmol, 1 eq.) in DCM (5 mL) was added DMP (1204.41 mg, 2.840 mmol, 2 eq.) in portions at 0° C. under a nitrogen atmosphere. The resulting mixture was stirred for 2 h at 0° C. under a nitrogen atmosphere. The resulting mixture was filtered and the filter cake was washed with DCM (3×50 mL). The filtrate was concentrated under reduced pressure. The residue was purified by Prep-TLC (PE/EA 2:1) to afford 138-2 (200 mg, 72.16%) as a white solid.

Step 3: Synthesis of 138-3. To a stirred solution of 138-2 (200 mg, 1.025 mmol, 1 eq.) in DCM (20 mL) was added DAST (1651.60 mg, 10.250 mmol, 10 eq.) in portions at −78° C. under a nitrogen atmosphere. The resulting mixture was stirred for 2 h at room temperature under a nitrogen atmosphere. The reaction was quenched by the addition of sat. NaHCO₃ (aq.) (2 mL) at 0° C. The aqueous layer was extracted with CH₂Cl₂ (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 70% gradient in 10 min; detector: UV 254 nm, to afford 138-3 (40 mg, 17.97%) as a yellow solid.

Step 4: Synthesis of 1-(4-(3,3-difluoro-1-methoxycyclobutyl)pyridin-2-yl)-N-(1-methyl-1H-indazol-7-yl)-1H-pyrazole-4-sulfonamide (I-138). A solution of 138-3 (76.61 mg, 0.276 mmol, 1.2 eq.), N-(1-methyl-1H-indazol-7-yl)-1H-pyrazole-4-sulfonamide (63.71 mg, 0.230 mmol, 1.0 eq.) and Cs₂CO₃ (225.02 mg, 0.690 mmol, 3 eq.) in DMF (5 mL) was stirred overnight at 90° C. under a nitrogen atmosphere. The resulting mixture was extracted with EtOAc (2×50 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 60% gradient in 10 min; detector: UV 254 nm. This resulted in 1-(4-(3,3-difluoro-1-methoxycyclobutyl)pyridin-2-yl)-N-(1-methyl-1H-indazol-7-yl)-1H-pyrazole-4-sulfonamide (14.4 mg, 12.83%) as a pink solid. (ES, m/z): [M+H]⁺ 475; ¹H NMR: (400 MHz, DMSO-d₆) δ 3.02 (s, 3H), 3.07-3.20 (m, 4H), 4.29 (s, 3H), 6.72 (d, J=6.8 Hz, 1H), 6.98 (t, J=8.0, 7.6 Hz, 1H), 7.54 (d, J=5.2, 1H), 7.71 (d, J=8.0 Hz, 1H), 7.96 (s, 1H), 8.05 (s, 1H), 8.09 (s, 1H), 8.59 (d, J=5.2 Hz, 1H), 8.84 (s, 1H), 10.04 (s, 1H).

### Example 320—Synthesis of 1-(4-(1,1-Difluoroethyl)Pyridin-2-yl)-N-(3-FLUORO-1-Methyl-1H-Indazol-7-yl)-1H-Pyrazole-4-Sulfonamide (I-139)

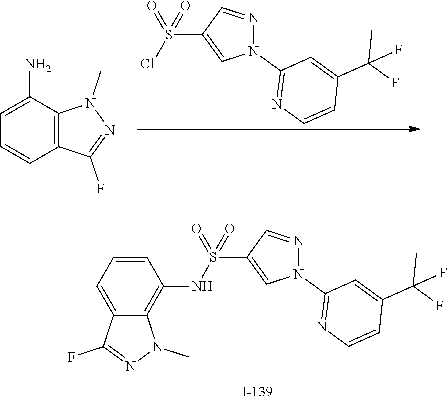 [SMILES: Cn1nc(F)c2cccc(N)c21 ; CC(F)(F)c1ccnc(-n2cc(S(=O)(=O)Cl)cn2)c1 ; Cn1nc(F)c2cccc(NS(=O)(=O)c3cnn(-c4cc(C(C)(F)F)ccn4)c3)c21]

A solution of 3-fluoro-1-methyl-1H-indazol-7-amine (50.0 mg, 0.303 mmol, 1.00 eq.) and 1-(4-(1,1-difluoroethyl)pyridin-2-yl)-1H-pyrazole-4-sulfonyl chloride (97.8 mg, 0.318 mmol, 1.05 eq.) in pyridine (2 mL) was stirred for 2 h at 80° C. under a nitrogen atmosphere. The mixture was allowed to cool down to room temperature. The resulting mixture was concentrated under reduced pressure. The residue was dissolved in water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃+0.05% NH₃·H₂O), mobile phase B: ACN; flow rate: 60 mL/min; gradient: 28% B to 51% B in 7 min; wavelength: 254 nm/220 nm; Rt(min): 6.15 to afford 1-(4-(1,1-difluoroethyl) pyridin-2-yl)-N-(3-fluoro-1-methyl-1H-indazol-7-yl)-1H-pyrazole-4-sulfonamide (12.8 mg, 9.66%) as an off-white solid. (ES, m/z): [M+H]⁺ 441; ¹H NMR: (400 MHz, DMSO-d₆) δ 2.06 (t, J=19.2 Hz, 3H), 4.16 (s, 3H), 6.82 (d, J=7.6 Hz, 1H), 7.02 (t, J=7.6 Hz, 1H), 7.59 (d, J=8.0 Hz, 1H), 7.67 (d, J=4.8 Hz, 1H), 8.07 (s, 2H), 8.69 (d, J=5.2 Hz, 1H), 8.85 (s, 1H), 10.16 (s, 1H).

### Example 321—Synthesis of 1-(4-(1,1-DIFLUOROPROPyl)Pyridin-2-yl)-N-(1-Methyl-1H-Indazol-7-yl)-1H-Pyrazole-4-Sulfonamide (I-140)

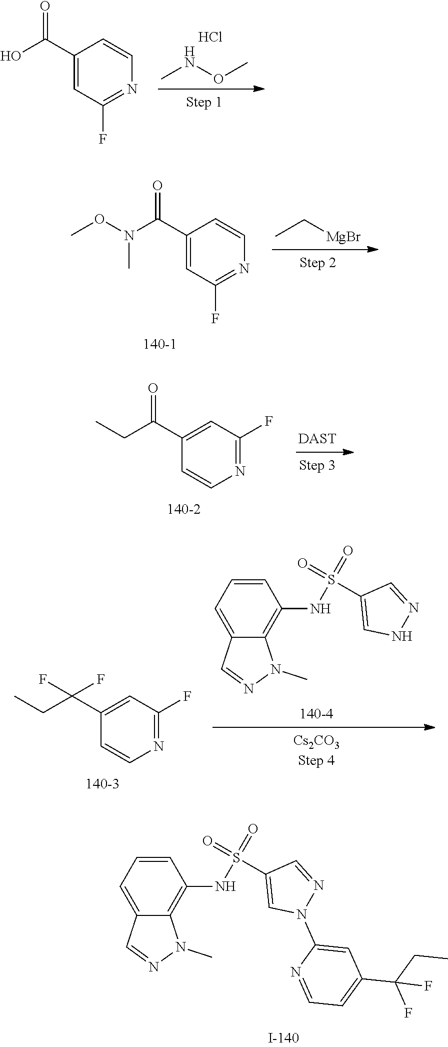

Step 1: Synthesis of 140-1. A mixture of 2-fluoropyridine-4-carboxylic acid (500 mg, 3.544 mmol, 1 eq.), N,O-dimethylhydroxylamine hydrochloride (525 mg, 5.382 mmol, 1.52 eq.), EDCI (649 mg, 4.180 mmol, 1.18 eq.), HOBt (574 mg, 4.248 mmol, 1.20 eq.) and DIEA (1374 mg, 10.631 mmol, 3.00 eq.) in THF (30 mL) was stirred overnight at room temperature under a nitrogen atmosphere. The residue was quenched with water (30 mL). The resulting mixture was extracted with EtOAc (3×40 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm, and to afford 140-1 (300 mg, 45.97%) as an off-white solid.

Step 2: Synthesis of 140-2. To a stirred solution of 140-1 (290 mg, 1.575 mmol, 1 eq.) in DCM (15 mL) was added ethylmagnesium bromide (629.56 mg, 4.725 mmol, 3 eq.) dropwise at −78° C. under a nitrogen atmosphere. The mixture was stirred for 2 h at −78° C. to room temperature under a nitrogen atmosphere. The reaction was quenched by the addition of sat. NH4Cl (aq.) (30 mL) at 0° C. The resulting mixture was extracted with CH₂Cl₂ (3×20 mL). The combined organic layers were washed with brine (10 mL) and dried over anhydrous Na₂SO₄. After filtration, the filtrate was concentrated under reduced pressure. The residue was purified by Prep-TLC (PE/EA 5:1) to afford 1-(2-fluoropyridin-4-yl)propan-1-one (212 mg, 87.91%) as a light yellow oil.

Step 3: Synthesis of 140-3. To a stirred solution of 140-2 (200 mg, 1.306 mmol, 1 eq.) in DCM (8 mL) was added DAST (519.91 mg, 3.226 mmol, 2.47 eq.) dropwise at 0° C. under a nitrogen atmosphere. The resulting mixture was stirred for 1 day at 40° C. under a nitrogen atmosphere. The resulting mixture was diluted with water (10 mL). The resulting mixture was extracted with EtOAc (3×15 mL). The combined organic layers were washed with brine (5 mL) and dried over anhydrous Na₂SO₄. After filtration, the filtrate was concentrated under reduced pressure. The residue was purified by prep-TLC (PE/EA 1:1) to afford 4-(1,1-difluoropropyl)-2-fluoropyridine (70 mg, 27.54%) as a light yellow solid.

Step 4: Synthesis of 1-(4-(1,1-difluoropropyl)pyridin-2-yl)-N-(1-methyl-1H-indazol-7-yl)-1H-pyrazole-4-sulfonamide (I-140). A mixture of 140-3 (60 mg, 0.343 mmol, 1 eq.) and N-(1-methylindazol-7-yl)-1H-pyrazole-4-sulfonamide (142.49 mg, 0.515 mmol, 1.5 eq.) and Cs₂CO₃ (334.83 mg, 1.029 mmol, 3 eq.) in DMSO (8 mL) was stirred for 4 h at 80° C. under a nitrogen atmosphere. The resulting mixture was diluted with water (30 mL). The resulting mixture was extracted with EtOAc (4×20 mL). The combined organic layers were washed with brine (5 mL) and dried over anhydrous Na₂SO₄. After filtration, the filtrate was concentrated under reduced pressure. The crude product was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 22% B to 49% B in 7 min; wavelength: 254 nm/220 nm; Rt(min): 7.95) to afford 1-(4-(1,1-difluoropropyl)pyridin-2-yl)-N-(1-methyl-1H-indazol-7-yl)-1H-pyrazole-4-sulfonamide (51.7 mg, 34.48%) as an off-white solid; (ES, m/z): [M+H]⁺ 433; ¹H NMR (400 MHz, DMSO-d₆) δ 0.95 (t, J=7.6 Hz, 3H), 2.26-2.35 (m, 2H), 4.29 (s, 3H), 6.73 (d, J=7.2 Hz, 1H), 6.97 (t, J=7.6 Hz, 1H), 7.63-7.69 (m, 2H), 8.03 (s, 1H), 8.07 (s, 1H), 8.08 (s, 1H), 8.68 (d, J=4.8 Hz, 1H), 8.84 (s, 1H), 10.07 (s, 1H).

### Example 322—Synthesis of 1-(4-(1,1-Difluoroethyl)Pyridin-2-yl)-N-(6-Methoxy-1-Methyl-1H-Pyrazolo[4,3-C]Pyridin-7-yl)-1H-Pyrazole-4-Sulfonamide (I-141)

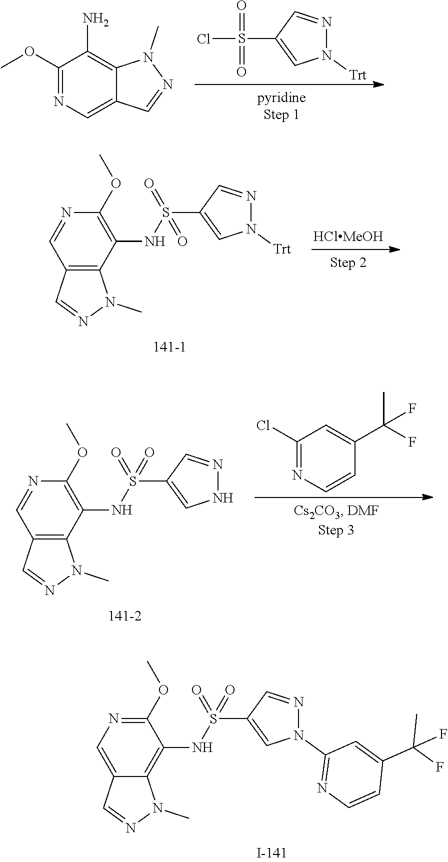

Step 1: Synthesis of I-141. A solution of 6-methoxy-1-methylpyrazolo[4,3-c]pyridin-7-amine (1.83 g, 10.270 mmol, 1 eq.) and 1-(triphenylmethyl)pyrazole-4-sulfonyl chloride (4.62 g, 11.297 mmol, 1.1 eq.) in pyridine (10 mL) was stirred overnight at room temperature. The resulting mixture was concentrated under reduced pressure. The residue was dissolved in water (30 mL) and extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 0% to 100% gradient in 20 min; detector: UV 254 nm. To get N-{6-methoxy-1-methylpyrazolo[4,3-c]pyridin-7-yl}-1-(triphenylmethyl)pyrazole-4-sulfonamide (2.8 g, 49.51%) as brown yellow solid.

Step 2: Synthesis of 141-2. A solution of N-{6-methoxy-1-methylpyrazolo[4,3-c]pyridin-7-yl}-1-(triphenylmethyl)pyrazole-4-sulfonamide (2.5 g, 4.540 mmol, 1 eq.) in HCl/MeOH (4M, 25 mL) was stirred overnight at room temperature. The residue was purified by reversed-phase flash chromatography with the following conditions: column: C18 silica gel; mobile phase: MeCN in water, 0% to 100% gradient in 20 min; detector: UV 254 nm, to give N-{6-methoxy-1-methylpyrazolo[4,3-c]pyridin-7-yl}-1H-pyrazole-4-sulfonamide (960 mg, 68.58%) as an off-white solid.

Step 3: Synthesis of 1-(4-(1,1-difluoroethyl)pyridin-2-yl)-N-(6-methoxy-1-methyl-1H-pyrazolo[4,3-c]pyridin-7-yl)-1H-pyrazole-4-sulfonamide (I-141). A solution of N-{6-methoxy-1-methylpyrazolo[4,3-c]pyridin-7-yl}-1H-pyrazole-4-sulfonamide (100 mg, 0.324 mmol, 1 eq.), Cs₂CO₃ (211.35 mg, 0.648 mmol, 2 eq.) and 2-chloro-4-(1,1-difluoroethyl)pyridine (86.39 mg, 0.486 mmol, 1.5 eq.) in DMF (2 mL) was stirred overnight at 140° C. under a nitrogen atmosphere. The resulting mixture was quenched with H₂O (10 mL) and extracted with EtOAc (3×10 mL). The combined organic layers were washed with brine (20 mL) and dried over anhydrous Na₂SO₄. After filtration, the filtrate was concentrated under reduced pressure. The crude product (100 mg) was purified by prep-HPLC with the following conditions: column: XBridge Prep OBD C18 column, 30*150 mm, 5 μm; mobile phase A: water (10 mmol/L NH₄CO₃)+0.05% NH₃·H₂O, mobile phase B: ACN; flow rate: 60 mL/min; gradient: 22% B to 48% B in 7 min; wavelength: 254 nm/220 nm; Rt(min): 7.43) to afford 1-[4-(1,1-difluoroethyl) pyridin-2-yl]-N-{6-methoxy-1-methylpyrazolo[4,3-c]pyridin-7-yl}pyrazole-4-sulfonamide (50.8 mg, 34.47%) as an off-white solid. (ES, m/z): [M+H]⁺ 450; ¹H NMR: ¹H NMR (400 MHz, DMSO-d) δ 1.99 (t, J=18.4 Hz, 3H), 3.49 (s, 3H), 4.31 (s, 3H), 7.53 (dd, J=5.2, 1.2 Hz, 1H), 7.91 (s, 1H), 8.11 (s, 1H), 8.12 (s, 1H), 8.57-8.58 (m, 2H), 8.79 (s, 1H).

### Example 323—Synthesis of N-(6-Methoxy-1-Methyl-1H-Indazol-7-yl)-1-(4-(1-Methoxy-3-Methylcyclobutyl)Pyridin-2-yl)-1H-Pyrazole-4-Sulfonamide (I-142)

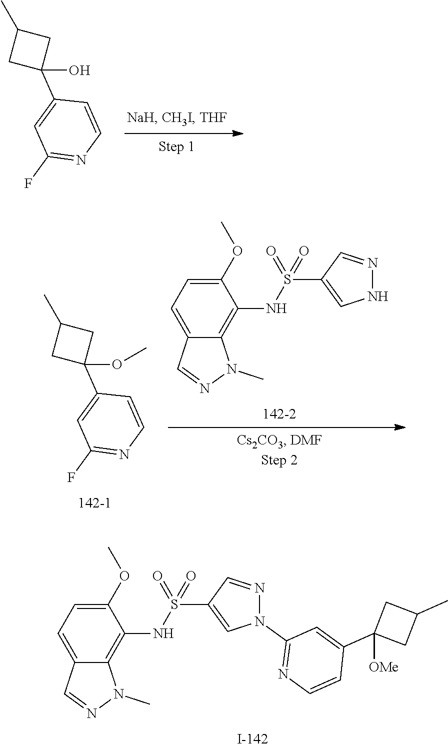

Step 1: Synthesis of 142-1. A solution of 1-(2-fluoropyridin-4-yl)-3-methylcyclobutan-1-ol (90.5 mg, 0.50 mmol, 1 eq.) in THF (5 mL) was treated with NaH (35.96 mg, 1.50 mmol, 3 eq.) for 30 min at 0° C. under a nitrogen atmosphere followed by the addition of CH₃I (708.87 mg, 4.99 mmol, 10 eq.) dropwise at 0° C. The resulting mixture was stirred overnight at room temperature. The reaction was quenched with water (20 mL) at 0° C. The aqueous layer was extracted with EtOAc (3×50 mL). The organic phase was combined and dried over with anhydrous Na₂SO₄. After filtration, the solution was concentrated under reduced pressure to give the crude product. The residue was purified by prep-TLC (PE/EA=4:1) to afford 142-1 (74 mg, 75.89%) as a yellow oil.

Step 2: Synthesis of N-(6-methoxy-1-methyl-1H-indazol-7-yl)-1-(4-(1-methoxy-3-methylcyclobutyl)pyridin-2-yl)-1H-pyrazole-4-sulfonamide (I-142). A solution of 142-1 (83.67 mg, 0.429 mmol, 1.3 eq.), Cs₂CO₃ (322.21 mg, 0.99 mmol, 3 eq.) and 142-2 (101.31 mg, 0.33 mmol, 1.00 eq.) in DMF (4 mL) was stirred for 2 days at 80° C. After the reaction was completed, the mixture was dissolved with water (20 mL) and extracted with EtOAc (4×30 mL). The organic layers were combined and dried over anhydrous Na₂SO₄. After filtration, the filtration was concentrated under vacuum to afford the crude product. The residue was purified by reversed-phase flash chromatography with the following conditions: column: C18 silica gel; mobile phase: ACN in water (10 mmol/L NH₄CO₃), 0% to 100% gradient in 40 min; detector: UV 254 nm, to afford N-(6-methoxy-1-methyl-1H-indazol-7-yl)-1-(4-(1-methoxy-3-methylcyclobutyl)pyridin-2-yl)-1H-pyrazole-4-sulfonamide (46.6 mg, 27.86%) as a light brown solid. (ES, m/z): [M+H]⁺ 483; ¹H NMR: (400 MHz, DMSO-d₆) δ 1.16 (d, J=6.0 Hz, 3H), 1.96-2.09 (m, 3H), 2.50-2.53 (m, 2H), 2.93 (s, 3H), 3.35 (s, 3H), 4.25 (s, 3H), 6.88 (d, J=8.8 Hz, 1H), 7.19-7.30 (m, 1H), 7.53 (dd, J=4.8, 1.2 Hz, 1H), 7.68 (d, J=8.8 Hz, 1H), 7.95-8.01 (m, 2H), 8.53 (d, J=5.2 Hz, 1H), 8.73 (s, 1H), 9.80 (s, 1H).

### Example 324—Synthesis of 1-(4-(1-Hydroxy-3-Methylcyclobutyl) Pyridin-2-yl)-N-(6-Methoxy-1-Methyl-1H-Indazol-7-yl)-1H-Pyrazole-4-Sulfonamide (I-143)

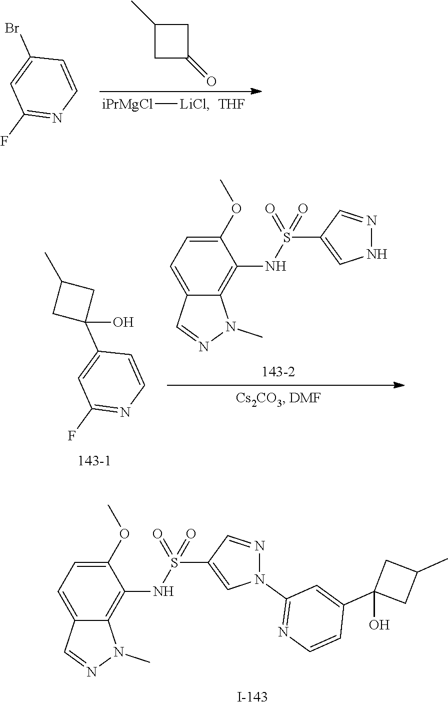

Step 1: Synthesis of 143-1. A solution of 4-bromo-2-fluoropyridine (1.75 g, 9.94 mmol, 1 eq.) in THE (100 mL) was treated with isopropylmagnesium chloride (1.53 g, 14.92 mmol, 1.5 eq.) for 30 min at −78° C. under a nitrogen atmosphere followed by the addition of 3-methylcyclobutan-1-one (1.00 g, 11.93 mmol, 1.20 eq.) dropwise at −60° C. Then the mixture was stirred from −60° C. to room temperature overnight. After the reaction was completed, the mixture was quenched with saturated NH₄Cl, extracted with EA, washed with the brine and dried over anhydrous Na₂SO₄ to give the crude product. The residue was purified by prep-TLC (PE/EA 4:1) to afford 143-1 (897 mg, 49.78%) as a yellow oil.

Step 2: Synthesis of 1-(4-(1-hydroxy-3-methylcyclobutyl)pyridin-2-yl)-N-(6-methoxy-1-methyl-1H-indazol-7-yl)-1H-pyrazole-4-sulfonamide (I-143). A solution of 143-1 (95.00 mg, 0.52 mmol, 1.5 eq.), 143-2 (106 mg, 0.35 mmol, 1 eq), and Cs₂CO₃ (341.62 mg, 1.05 mmol, 3 eq.) in DMF (5 mL) was stirred for 2 days at 80° C. After the reaction was completed, the mixture was purified by reversed-phase flash chromatography with the following conditions: column: C18 silica gel; mobile phase: ACN in water (10 mmol/L NH₄CO₃), 0% to 100% gradient in 40 min; detector: UV 254 nm, to afford 1-(4-(1-hydroxy-3-methylcyclobutyl)pyridin-2-yl)-N-(6-methoxy-1-methyl-1H-indazol-7-yl)-1H-pyrazole-4-sulfonamide (20.3 mg, 11.93%) as an off-white solid. (ES, m/z): [M+H]⁺⁺ 483; ¹H NMR: (400 MHz, DMSO-d,ppm) δ 1.16 (d, J=6.0 Hz, 3H), 2.01-2.24 (m, 3H), 2.50-2.53 (m, 2H), 2.93 (s, 3H), 4.25 (s, 3H), 6.00 (s, 1H), 6.88 (d, J=8.8 Hz, 1H), 7.49-7.64 (m, 2H), 7.96-8.06 (m, 4H), 8.46 (d, J=5.2 Hz, 1H), 8.69 (s, 1H).

### Example 325—Synthesis of 1-(4-(1-Fluorocyclobutyl)Pyridin-2-yl)-N-(6-Methoxy-1-Methyl-1H-Indazol-7-yl)-1H-Pyrazole-4-Sulfonamide (I-144)

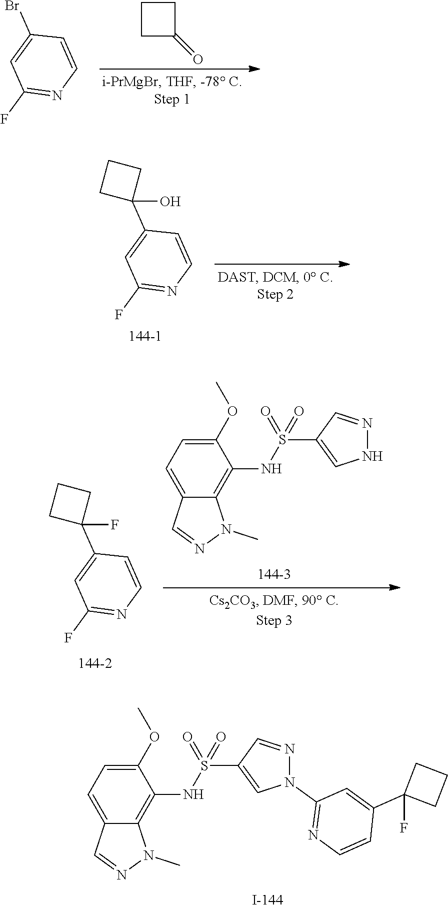 [SMILES: Fc1cc(Br)ccn1 ; O=C1CCC1 ; OC1(c2ccnc(F)c2)CCC1 ; Fc1cc(C2(F)CCC2)ccn1 ; COc1ccc2cnn(C)c2c1NS(=O)(=O)c1cn[nH]c1 ; COc1ccc2cnn(C)c2c1NS(=O)(=O)c1cnn(-c2cc(C3(F)CCC3)ccn2)c1]

Step 1: Synthesis of 144-1. A solution of 4-bromo-2-fluoropyridine (5 g, 28.411 mmol, 1 eq.) in THE (33 mL) was treated with a solution of i-PrMgBr LiCl in THE (33 mL, 1.3 M, 1.5 eq.) for 30 min at −78° C. under a nitrogen atmosphere followed by the addition of cyclobutanone (3.98 g, 56.822 mmol, 2 eq.) dropwise at −78° C. The resulting mixture was stirred for 2 h at −78° C. under a nitrogen atmosphere. The reaction was quenched with sat. NH₄Cl (aq.) at room temperature. The resulting mixture was extracted with CH₂Cl₂ (3×30 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 40% to 43% gradient in 20 min; detector: UV 215 nm. This resulted in 144-1 (3.8 g, 72.00%) as a light yellow oil.

Step 2: Synthesis of 144-2. To a stirred solution of 144-1 (925 mg, 5.533 mmol, 1 eq.) in DCM (20 mL, 314.612 mmol, 56.86 eq.) was added DAST (1.5 mL, 11.353 mmol, 2.05 eq.) dropwise at 0° C. under a nitrogen atmosphere. The resulting mixture was stirred for 2 h at 0° C. under a nitrogen atmosphere and then warmed to room temperature. The resulting mixture was concentrated under reduced pressure. The residue was purified by reversed-phase flash chromatography with the following conditions: column: C18 silica gel; mobile phase: MeCN in water (10 mmol/L NH₄CO₃), 55% to 60% gradient in 30 min; detector: UV 254 nm. This resulted in 144-2 (185 mg, 19.76%) as a light yellow liquid.

Step 3: Synthesis of 1-(4-(1-fluorocyclobutyl)pyridin-2-yl)-N-(6-methoxy-1-methyl-1H-indazol-7-yl)-1H-pyrazole-4-sulfonamide (I-144). To a stirred solution of 144-3 (120 mg, 0.390 mmol, 1.00 eq.) and Cs₂CO₃ (382 mg, 1.172 mmol, 3.00 eq.) in DMF (6 mL) was added 144-2 (132.1 mg, 0.781 mmol, 2.00 eq.) dropwise at room temperature under a nitrogen atmosphere. The resulting mixture was stirred for 3 h at 90° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄CO₃), 50% to 55% gradient in 30 min; detector: UV 254 nm. This resulted in 1-(4-(1-fluorocyclobutyl)pyridin-2-yl)-N-(6-methoxy-1-methyl-1H-indazol-7-yl)-1H-pyrazole-4-sulfonamide (50 mg, 27.21%) as a light brown solid. (ES, m/z): [M+H]⁺ 457; ¹H NMR: (400 MHz, DMSO-d₆) δ 1.87-1.89 (m, 1H), 2.08-2.12 (m, 1H), 2.59-2.69 (m, 4H), 3.33 (s, 3H), 4.24 (s, 3H), 6.88 (d, J=8.8 Hz, 1H), 7.60 (d, J=5.2 Hz, 1H), 7.68 (d, J=8.8 Hz, 1H), 7.98 (s, 2H), 8.02 (s, 1H), 8.58 (d, J=5.2 Hz, 1H), 8.73 (s, 1H), 9.82 (s, 1H).

### Example 326—Synthesis of N-(1-Methyl-1H-Indazol-7-yl)-1-(Pyridin-2-yl)-1H-Pyrazole-4-Sulfonamide (I-145)

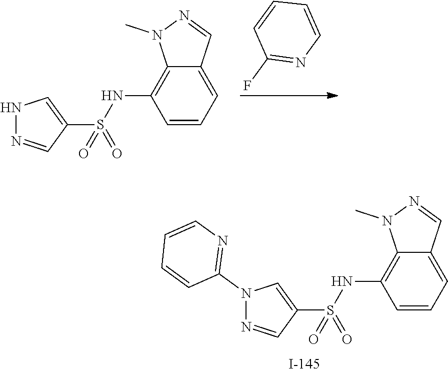

To a stirred solution of N-(1-methylindazol-7-yl)-1H-pyrazole-4-sulfonamide (100 mg, 0.361 mmol, 1 eq.) and 2-fluoropyridine (35.01 mg, 0.361 mmol, 1 eq.) in DMF (1 mL) was added Cs₂CO₃ (352.49 mg, 1.083 mmol, 3 eq.) under a nitrogen atmosphere. The resulting mixture was stirred overnight at 140° C. under a nitrogen atmosphere. The resulting mixture was quenched with H₂O (10 mL) and extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in N-(1-methyl-1H-indazol-7-yl)-1-(pyridin-2-yl)-1H-pyrazole-4-sulfonamide (23.2 mg, 18.03%) as a white solid. (ES, m/z): [M+H]+ 355; ¹H NMR: (400 MHz, DMSO-d₆) δ 4.29 (s, 3H), 6.72 (d, J=7.2 Hz, 1H), 6.98 (t, J=7.6 Hz, 1H), 7.47-7.50 (m, 1H), 7.69 (d, J=8.4 Hz, 1H), 7.98-8.13 (m, 4H), 8.53 (d, J=4.4 Hz, 1H), 8.80 (s, 1H), 10.02 (s, 1H).

### Example 327—Synthesis of 1-(4-Methoxypyridin-2-yl)-N-(1-Methyl-1H-Indazol-7-yl)-1H-Pyrazole-4-Sulfonamide (I-146)

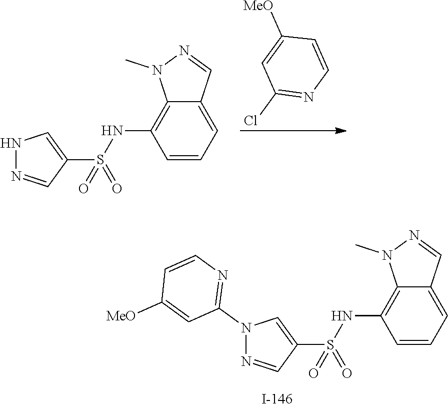

A solution of N-(1-methylindazol-7-yl)-1H-pyrazole-4-sulfonamide (100 mg, 0.361 mmol, 1 eq.), 2-chloro-4-methoxypyridine (77.66 mg, 0.541 mmol, 1.5 eq.), CuI (68.68 mg, 0.361 mmol, 1 eq.), N1,N2-dimethylcyclohexane-1,2-diamine (51.30 mg, 0.361 mmol, 1 eq.) and Cs₂CO₃ (352.49 mg, 1.083 mmol, 3 eq.) in DMF (1.5 mL) was stirred for 16 h at 110° C. under a nitrogen atmosphere. The resulting mixture was quenched with H₂O (10 mL) and extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄CO₃), 10% to 50% gradient in 10 min; detector: UV 254 nm. This resulted in 1-(4-methoxypyridin-2-yl)-N-(1-methylindazol-7-yl)pyrazole-4-sulfonamide (24.4 mg, 17.43%) as a white solid. (ES, m/z): [M+H]⁺ 385; ¹H NMR: (400 MHz, DMSO-d₆) δ 3.95 (s, 3H), 4.29 (s, 3H), 6.72 (d, J=7.2 Hz, 1H), 6.96 (t, J=7.6 Hz, 1H), 7.05 (dd, J=5.6, 2.4 Hz, 1H), 7.47 (d, J=2.4 Hz, 1H), 7.64 (d, J=8.0 Hz, 1H), 7.99 (s, 1H), 8.06 (s, 1H), 8.32 (d, J=5.6 Hz, 1H), 8.76 (s, 1H), 10.01 (s, 1H).

### Example 328—Synthesis of 1-(4-(Azetidin-1-yl)Pyridin-2-yl)-N-(1-Methyl-1H-Indazol-7-yl)-1H-Pyrazole-4-Sulfonamide (I-248)

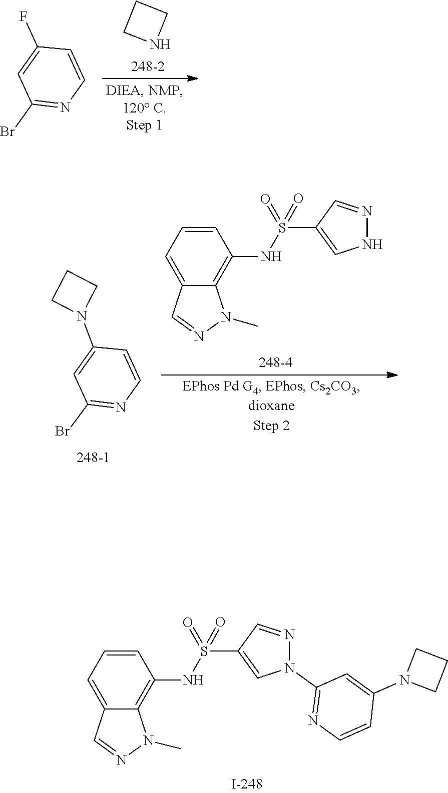

Step 1: Synthesis of 4-(azetidin-1-yl)-2-bromopyridine (248-1): A solution of 2-bromo-4-fluoropyridine (240 mg, 1.364 mmol, 1 equiv) in NMP (5 mL) was treated with DIEA (528.77 mg, 4.092 mmol, 3 equiv) and azetidine (116.80 mg, 2.05 mmol, 1.5 equiv) for 2 h at 120° C. under a nitrogen atmosphere. The reaction was quenched with water (50 mL) at room temperature. The aqueous layer was extracted with DCM (3×100 mL). The organic phase was combined and concentrated under reduced pressure. The residue was purified by reversed-phase flash chromatography with the following conditions: column: C₁₈ silica gel; mobile phase: ACN in water (10 mmol/L NH₄HCO₃), 10% to 50% gradient in 10 min; detector: UV 254 nm. This resulted in 248-1 (280 mg, 96.36%) as a white solid.

Step 2: Synthesis of 1-(4-(azetidin-1-yl)pyridin-2-yl)-N-(1-methyl-1H-indazol-7-yl)-1H-pyrazole-4-sulfonamide (I-248): A solution of 248-1 (105 mg, 0.493 mmol, 1 equiv), N-(1-methylindazol-7-yl)-1H-pyrazole-4-sulfonamide (177.64 mg, 0.641 mmol, 1.3 equiv), EPhos (52.71 mg, 0.099 mmol, 0.2 equiv), EPhos Pd G₄ (45.27 mg, 0.049 mmol, 0.1 equiv) and Cs₂CO₃ (481.67 mg, 1.479 mmol, 3 equiv) in 1,4-dioxane (5 mL) was stirred overnight at 90° C. under a nitrogen atmosphere. After the reaction was complete, the mixture was quenched with water (100 mL) at room temperature. The aqueous layer was extracted with EtOAc (3×100 mL). The organic phase was combined and concentrated under reduced pressure. The residue was purified by reversed-phase flash chromatography with the following conditions: column: C₁₈ silica gel; mobile phase: ACN in water (0.1% NH₃·H₂O), 25% to 50% gradient in 20 min; detector: UV 254 nm. This resulted in 1-(4-(azetidin-1-yl)pyridin-2-yl)-N-(1-methyl-1H-indazol-7-yl)-1H-pyrazole-4-sulfonamide (I-248, 21 mg, 10.16%) as a white solid. LCMS (ES, m/z): [M+H]⁺=410.10; ¹H NMR: (400 MHz, DMSO-d₆) δ 2.32-2.44 (m, 2H), 4.02 (t, J=7.6 Hz, 4H), 4.27 (s, 3H) 6.37 (dd, J=1.8, 6.0 Hz, 1H), 6.69 (d, J=7.6 Hz, 1H), 6.81 (d, J=2.0 Hz, 1H), 6.97 (t, J=7.6 Hz, 1H), 7.68 (d, J=8.0 Hz, 1H), 7.93 (s, 1H), 8.02 (d, J=6.4 Hz, 1H), 8.08 (s, 1H), 8.70 (s, 1H), 9.97 (s, 1H).

### Example 329—Synthesis of N-(1-Methyl-1H-Indazol-7-yl)-1-(4-(Pyrrolidin-1-yl)Pyridin-2-yl)-1H-Pyrazole-4-Sulfonamide (I-249)

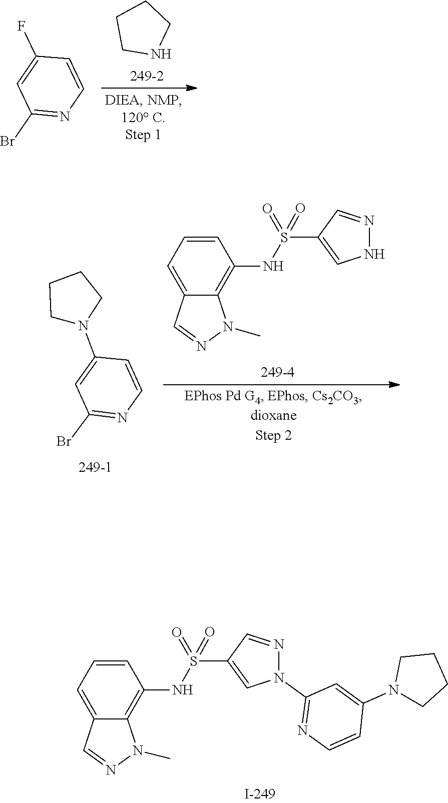 [SMILES: Fc1ccnc(Br)c1 ; C1CCNC1 ; Brc1cc(N2CCCC2)ccn1 ; Cn1ncc2cccc(NS(=O)(=O)c3cn[nH]c3)c21 ; Cn1ncc2cccc(NS(=O)(=O)c3cnn(-c4cc(N5CCCC5)ccn4)c3)c21]

Step 1: Synthesis of 2-bromo-4-(pyrrolidin-1-yl)pyridine (249-1): A solution of 2-bromo-4-fluoropyridine (220 mg, 1.25 mmol, 1 equiv) in NMP (2 mL) was treated with pyrrolidine (177.82 mg, 2.50 mmol, 2 equiv) and DIEA (484.71 mg, 3.750 mmol, 3 equiv) for 2 h at 120° C. under a nitrogen atmosphere. The desired product could be detected by LCMS. The reaction was diluted with water (20 mL) at room temperature. The aqueous layer was extracted with DCM (3×50 mL). The organic layers were combined, washed with the brine (100 mL) and concentrated under vacuum to give the crude. The residue was purified by reversed-phase flash chromatography with the following conditions: column: C₁₈ silica gel; mobile phase: ACN in water (10 mmol/L NH₄HCO₃), 30% to 50% gradient in 15 min; detector: UV 254 nm. This resulted in 249-1 (280 mg, 98.63%) as a white solid.

Step 2: Synthesis of N-(1-methyl-1H-indazol-7-yl)-1-(4-(pyrrolidin-1-yl)pyridin-2-yl)-1H-pyrazole-4-sulfonamide (I-249): A solution of 249-1 (105 mg, 0.462 mmol, 1 equiv), Cs₂CO₃ (451.92 mg, 1.386 mmol, 3 equiv), EPhos Pd G₄ (42.47 mg, 0.046 mmol, 0.1 equiv), EPhos (49.45 mg, 0.092 mmol, 0.2 equiv), and N-(1-methylindazol-7-yl)-1H-pyrazole-4-sulfonamide (166.67 mg, 0.601 mmol, 1.3 equiv) in 1,4-dioxane (4 mL) was stirred overnight at 90° C. under a nitrogen atmosphere. The reaction was diluted with water (50 mL). The aqueous layer was extracted with EtOAc (3×100 mL). The organics were combined, washed with the brine (100 mL) and concentrated under vacuum to give the crude. The residue was purified by reversed-phase flash chromatography with the following conditions: column: C₁₈ silica gel; mobile phase: ACN in water (0.1% NH₃·H₂O), 10% to 50% gradient in 20 min; detector: UV 254 nm to give the crude. The crude product (120 mg) was purified by Prep-HPLC with the following conditions (column: XBridge Prep OBD C₁₈ column, 30*150 mm, 5 μm; mobile phase A: water (10 mmol/L NH₄HCO₃)+0.05% NH₃·H₂O, mobile phase B: ACN; flow rate: 60 mL/min; gradient: 16% B to 43% B in 7 min; wave length: 254 nm/220 nm; RT₁(min): 8.27) to afford N-(1-methyl-1H-indazol-7-yl)-1-(4-(pyrrolidin-1-yl)pyridin-2-yl)-1H-pyrazole-4-sulfonamide (I-249, 20.4 mg, 10.40%) as a white solid. LCMS (ES, m/z): [M+H]⁺=424.15; ¹H NMR: (400 MHz, DMSO-d₆) δ 1.97-2.07 (m, 4H), 3.30-3.38 (m, 4H), 4.27 (s, 3H), 6.53 (d, J=2.0, 6.0 Hz, 1H), 6.69 (d, J=7.2, 1H), 6.93-6.99 (m, 2H), 7.68 (d, J=8.0 Hz, 1H), 7.93 (s, 1H), 8.01 (d, J=6.0 Hz, 1H), 8.08 (s, 1H), 8.71 (s, 1H), 9.96 (s, 1H).

### Example 330—Synthesis of 1-(4-(1-ACETyl-3-Fluoroazetidin-3-yl)Pyridin-2-yl)-N-(1-Methyl-1H-Indazol-7-yl)-1H-Pyrazole-4-Sulfonamide (I-253)

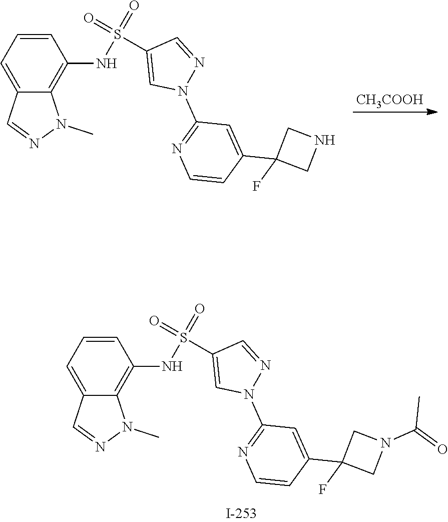 [SMILES: Cn1ncc2cccc(NS(=O)(=O)c3cnn(-c4cc(C5(F)CNC5)ccn4)c3)c21 ; "CH3COOH" ; CC(=O)N1CC(F)(c2ccnc(-n3cc(S(=O)(=O)Nc4cccc5cnn(C)c45)cn3)c2)C1]

To a stirred solution/mixture of 1-[4-(3-fluoroazetidin-3-yl)pyridin-2-yl]-N-(1-methylindazol-7-yl)pyrazole-4-sulfonamide (137 mg, 0.320 mmol, 1 equiv) and EDCI (92.16 mg, 0.480 mmol, 1.5 equiv) and HOBt (64.96 mg, 0.480 mmol, 1.5 equiv) in DMF (8 mL) were added acetic acid (23.10 mg, 0.384 mmol, 1.2 equiv) and DMAP (7.83 mg, 0.064 mmol, 0.2 equiv) in portions at room temperature under a nitrogen atmosphere. The resulting mixture was stirred for 4 h at room temperature under a nitrogen atmosphere. The resulting mixture was diluted with water (80 mL) and extracted with DCM (3×50 mL). The combined organic layers were washed with brine (50 mL), and dried over anhydrous Na₂SO₄. After filtration, the filtrate was concentrated under reduced pressure. The crude product (200 mg) was purified by Prep-HPLC with the following conditions (column: XBridge Prep OBD C₁₈ column, 30*150 mm, 5 m; mobile phase A: water (10 mmol/L NH₄HCO₃)+0.05% NH₃·H₂O, mobile phase B: ACN; flow rate: 60 mL/min; gradient: 11% B to 30% B in 7 min; wave length: 254 nm/220 nm; RT₁(min): 7.2) to afford 1-(4-(1-acetyl-3-fluoroazetidin-3-yl)pyridin-2-yl)-N-(1-methyl-1H-indazol-7-yl)-1H-pyrazole-4-sulfonamide (I-253, 45.6 mg, 30.18%) as an off-white solid. LCMS (ES, m/z): [M+H]⁺=470; ¹H NMR: (400 MHz, DMSO-d₆) 1.90 (s, 3H), 4.29 (s, 3H), 4.34 (d, J=3.2 Hz, 2H), 4.63-4.70 (m, 2H), 6.72 (d, J=7.2 Hz, 1H), 6.95 (t, J=7.6 Hz, 1H), 7.65 (d, J=5.2, 2H), 8.05 (s, 3H), 8.62 (d, J=5.2 Hz, 1H), 8.82 (s, 1H), δ 10.06 (d, J=6.8 Hz, 1H).

### Example 331—Synthesis of 1-(4-(2-Hydroxy-1,3-Dimethoxypropan-2-yl)Pyridin-2-yl)-N-(1-Methyl-1H-Indazol-7-yl)-1H-Pyrazole-4-Sulfonamide (I-257)

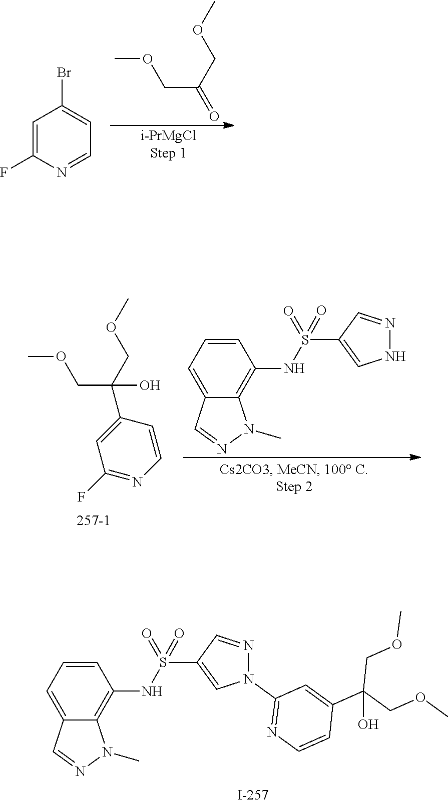

Step 1: Synthesis of 2-(2-fluoropyridin-4-yl)-1,3-dimethoxypropan-2-ol (257-1): A solution of 4-bromo-2-fluoropyridine (1 g, 5.682 mmol, 1 equiv) in THE (10 mL) was treated with chloro(isopropyl)magnesium (13.11 mL, 17.046 mmol, 3 equiv) for 1 h at −78° C. under a nitrogen atmosphere followed by the addition of 1,3-dimethoxypropan-2-one (2.01 g, 17.046 mmol, 3 equiv) in THE (15 mL) in portions at −60° C. The resulting mixture was stirred for 2 h at −60° C. under a nitrogen atmosphere. The reaction was quenched with 1N HCl (aq.) at 0° C. The mixture was basified to pH 7 with saturated NaHCO₃ (aq.). The resulting mixture was extracted with DCM (3×100 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 30% to 35% gradient in 10 min; detector: UV 254 nm. The resulting mixture was concentrated under reduced pressure. This resulted in 2-(2-fluoropyridin-4-yl)-1,3-dimethoxypropan-2-ol (257-1, 900 mg, 73.59%) as a light yellow oil.

Step 2: Synthesis of 1-(4-(2-hydroxy-1,3-dimethoxypropan-2-yl)481yridine-2-yl)-N-(1-methyl-1H-indazol-7-yl)-1H-pyrazole-4-sulfonamide (I-257): To a stirred solution of 2-(2-fluoropyridin-4-yl)-1,3-dimethoxypropan-2-ol (120 mg, 0.558 mmol, 1 equiv) and N-(1-methylindazol-7-yl)-1H-pyrazole-4-sulfonamide (185.53 mg, 0.670 mmol, 1.2 equiv) in I (3 mL) were added Cs₂CO₃ (544.99 mg, 1.674 mmol, 3 equiv) and potassium 2-methylpropan-2-olate (187.69 mg, 1.674 mmol, 3 equiv). The resulting mixture was stirred for 16 h at 100° C. under a nitrogen atmosphere. The reaction was quenched with water at room temperature. The resulting mixture was extracted with EtOAc (2×50 mL). The combined organic layers were washed with brine (1×30 mL) and dried over anhydrous Na₂SO₄. After filtration, the filtrate was concentrated under reduced pressure. The crude product (80 mg) was purified by Prep-HPLC with the following conditions (column: Xbridge Prep OBD C18 column, 30*150 mm, 5 μm; mobile phase A: water (10 mmol/L NH₄HCO₃+0.05% NH₃H₂O), mobile phase B: I; flow rate: 60 mL/min; gradient: 19% B to 44% B in 7 min; wave length: 220 nm; RT1(min): 5.92) to afford 1-[4-(2-hydroxy-1,3-dimethoxypropan-2-yl)482yridine-2-yl]-N-(1-methylindazol-7-yl)pyrazole-4-sulfonamide (I-257, 22.5 mg, 8.45%) as a white solid. LCMS (ES, m/z): [M+H]⁺=473; ¹H NMR: (400 MHz, DMSO-d₆) δ 3.25 (s, 6H), 3.51 (d, J=9.8 Hz, 2H), 3.62 (d, J=9.8 Hz, 2H), 4.29 (s, 3H), 5.75 (s, 1H), 6.71 (d, J=7.2 Hz, 1H), 6.98 (t, J=7.8 Hz, 1H), 7.52 (m, J=5.4, 1.4 Hz, 1H), 7.68 (s, 1H), 8.01 (s, 1H), 8.06-8.13 (m, 2H), 8.43 (d, J=5.2 Hz, 1H), 8.78 (s, 1H), 10.02 (s, 1H).

### Example 332—Synthesis of N-(6-Methoxy-1-Methyl-1H-Indazol-7-yl)-1-(4-(2-Methoxypropan-2-yl)Pyridin-2-yl)-1H-Pyrazole-4-Sulfonamide (I-258)

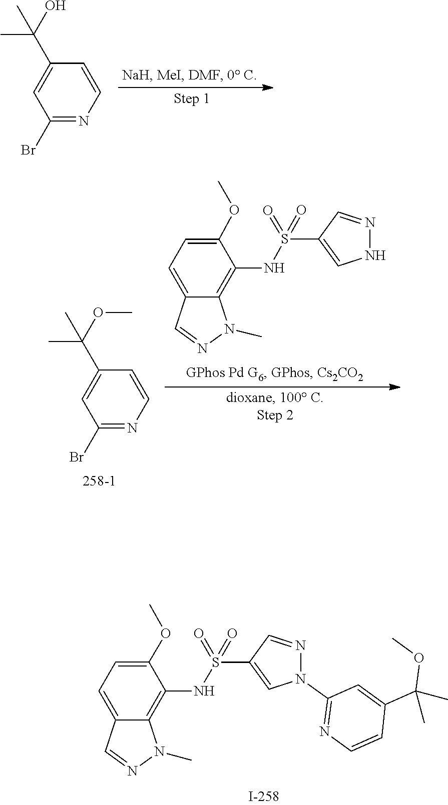

Step 1: Synthesis of 2-bromo-4-(2-methoxypropan-2-yl)pyridine (258-1): A solution of 2-(2-bromopyridin-4-yl)propan-2-ol (1 g, 4.628 mmol, 1 equiv) in dimethylformamide (20 mL) was treated with NaH (0.17 g, 6.942 mmol, 1.5 equiv) for 30 min at 0° C. under a nitrogen atmosphere followed by the addition of Mel (1.31 g, 9.256 mmol, 2 equiv) in portions at room temperature. The resulting mixture was stirred for 2 h at room temperature under a nitrogen atmosphere. The reaction was quenched with sat. NH₄Cl (aq.) at room temperature. The resulting mixture was extracted with EtOAc (2×50 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 60% gradient in 10 min; detector: UV 254 nm. to afford 2-bromo-4-(2-methoxypropan-2-yl)pyridine (258-1, 1 g, 93.90%) as a yellow oil.

Step 2: Synthesis of N-(6-methoxy-1-methyl-1H-indazol-7-yl)-1-(4-(2-methoxypropan-2-yl)pyridin-2-yl)-1H-pyrazole-4-sulfonamide (I-258): To a stirred mixture of 2-bromo-4-(2-methoxypropan-2-yl)pyridine (67.39 mg, 0.292 mmol, 1.5 equiv) and N-(6-methoxy-1-methylindazol-7-yl)-1H-pyrazole-4-sulfonamide (60 mg, 0.195 mmol, 1.00 equiv) in dioxane (5 mL) were added Cs₂CO₃ (190.83 mg, 0.585 mmol, 3 equiv), Gphos Pd G6 (18.51 mg, 0.020 mmol, 0.1 equiv) and [3-(tert-butoxy)-6-methoxy-2′,6′-bis(propan-2-yl)-[1,1′-biphenyl]-2-yl]dicyclohexylphosphane (10.48 mg, 0.020 mmol, 0.1 equiv) in portions at room temperature under a nitrogen atmosphere. The resulting mixture was stirred for 5 h at 100° C. under a nitrogen atmosphere. The resulting mixture was diluted with water (20 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-(6-methoxy-1-methylindazol-7-yl)-1-[4-(2-methoxypropan-2-yl)pyridin-2-yl]pyrazole-4-sulfonamide (I-258, 34.6 mg, 38.63%) as a white solid. LCMS (ES, m/z): [M+H]⁺=457; ¹H NMR: (400 MHz, DMSO-d₆): δ 1.50 (s, 6H), 3.11 (s, 3H), 3.35 (s, 3H), 4.24 (s, 3H), 6.88 (d, J=8.8 Hz, 1H), 7.47 (dd, J=5.2, 1.6 Hz, 1H), 7.68 (d, J=8.8 Hz, 1H), 7.93-8.02 (m, 3H), 8.50 (d, J=5.4 Hz, 1H), 8.71 (s, 1H), 9.80 (s, 1H).

### Example 333—Synthesis of N-(1-Methyl-1H-Indazol-7-yl)-1-(4-(1,2,3-Trimethoxypropan-2-yl)Pyridin-2-yl)-1H-Pyrazole-4-Sulfonamide (I-259)

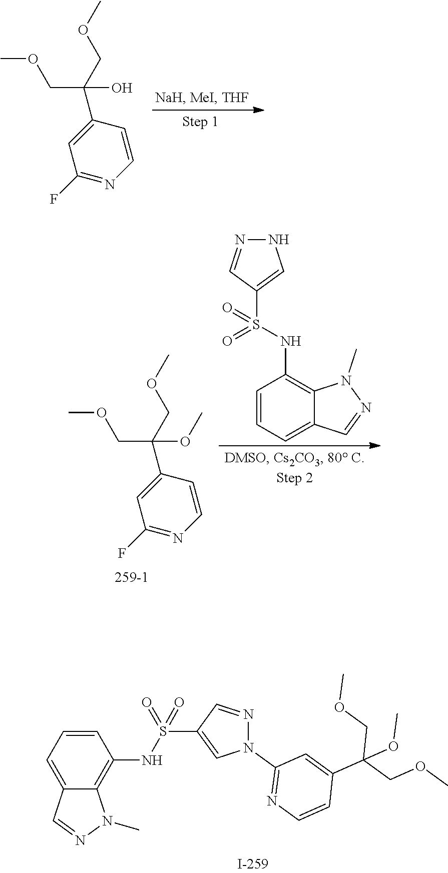

Step 1: Synthesis of 259-1: A solution of 2-(2-fluoropyridin-4-yl)-1,3-dimethoxypropan-2-ol (150 mg, 0.697 mmol, 1 equiv) in THE (5 mL) was treated with NaH (25.09 mg, 1.045 mmol, 1.5 equiv) for 30 min at 0° C. under a nitrogen atmosphere followed by the addition of Mel (197.85 mg, 1.394 mmol, 2.00 equiv) in portions at room temperature. The resulting mixture was stirred for 2 h at 50° C. under a nitrogen atmosphere. The reaction was quenched with sat. NH₄Cl (aq.) at room temperature. The resulting mixture was extracted with EtOAc (2×50 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm to afford 2-fluoro-4-(1,2,3-trimethoxypropan-2-yl)pyridine (259-1, 80 mg, 50.07%) as a white solid.

Step 2. Synthesis of Synthesis of N-(1-methyl-1H-indazol-7-yl)-1-(4-(1,2,3-trimethoxypropan-2-yl)pyridin-2-yl)-1H-pyrazole-4-sulfonamide (I-259): To a stirred solution of 2-fluoro-4-(1,2,3-trimethoxypropan-2-yl)pyridine (80 mg, 0.349 mmol, 1 equiv) and N-(1-methylindazol-7-yl)-1H-pyrazole-4-sulfonamide (96.77 mg, 0.349 mmol, 1.00 equiv) in DMSO (2 mL) was added Cs₂CO₃ (341.10 mg, 1.047 mmol, 3.00 equiv) in portions at room temperature under a nitrogen atmosphere. The resulting mixture was stirred for 2 h at 80° C. under a nitrogen atmosphere. The reaction was quenched with sat. NH₄Cl (aq.) at room temperature. The resulting mixture was extracted with EtOAc (3×50 mL). The combined organic layers were washed with brine (20 mL) and dried over anhydrous Na₂SO₄. After filtration, the filtrate was concentrated under reduced pressure. The crude product (100 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 12% B to 37% B in 7 min; wave length: 220 nm; RT1(min): 7.88) to afford N-(1-methylindazol-7-yl)-1-[4-(1,2,3-trimethoxypropan-2-yl)pyridin-2-yl]pyrazole-4-sulfonamide (I-259, 20.9 mg, 12.28%) as a white solid. LCMS (ES, m/z): [M+H]=487; ¹H NMR: ¹H NMR (400 MHz, DMSO-d₆): δ 3.24 (m, J=6.6 Hz, 9H), 3.64-3.75 (m, 4H), 4.32 (s, 3H), 6.74 (d, J=7.4 Hz, 1H), 6.88-6.96 (m, 1H), 7.41-7.47 (m, 1H), 7.51 (s, 1H), 7.94 (d, J=1.4 Hz, 1H), 8.00 (d, J=7.6 Hz, 2H), 8.47 (d, J=5.2 Hz, 1H), 8.75 (s, 1H), 10.05 (s, 1H).

### Example 334—Synthesis of 1-(4-(2-Hydroxypropan-2-yl)Pyridin-2-yl)-N-(6-Methoxy-1-Methyl-1H-Pyrazolo[4,3-C]Pyridin-7-yl)-1H-Pyrazole-4-Sulfonamide (I-260)

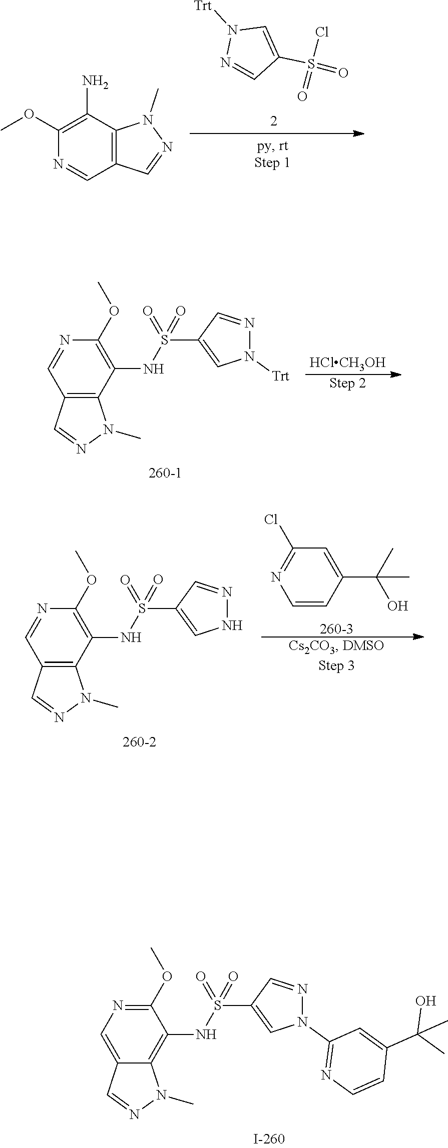

Step 1: Synthesis of 260-1: A solution of 6-methoxy-1-methylpyrazolo[4,3-c]pyridin-7-amine (1 g, 5.612 mmol, 1 equiv) and 1-(triphenylmethyl)pyrazole-4-sulfonyl chloride (3.44 g, 8.418 mmol, 1.5 equiv) in pyridine (50 mL) was stirred overnight at room temperature. The resulting mixture was concentrated under reduced pressure. The residue was dissolved in MeCN (30 mL). The resulting mixture was filtered and the filter cake was washed with MeCN (3×5 mL). The filtrate was concentrated under reduced pressure to give 260-1 (2.8 g, 90.61%) as yellow solid.

Step 2: Synthesis of 260-2: A solution of 260-1 (1 g, 1.816 mmol, 1 equiv) in HCl (g) in MeOH (15 mL) was stirred overnight at room temperature. The resulting mixture was concentrated under reduced pressure to give 260-2 (535 mg, 95.55%) as purple solid.

Step 3: Synthesis of 1-(4-(2-hydroxypropan-2-yl)pyridin-2-yl)-N-(6-methoxy-1-methyl-1H-pyrazolo[4,3-c]pyridin-7-yl)-1H-pyrazole-4-sulfonamide (I-260): A solution of 260-2 (100 mg, 0.324 mmol, 1 equiv), Cs₂CO₃ (211.35 mg, 0.648 mmol, 2 equiv) and 2-(2-chloropyridin-4-yl)propan-2-ol (83.49 mg, 0.486 mmol, 1.5 equiv) in DMSO (3 mL) was stirred for 6 h at 140° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by Prep-HPLC with the following conditions (column: XBridge Prep OBD C18 column, 30*150 mm, 5 μm; mobile phase A: water (10 mmol/L NH₄HCO₃+0.05% oNH3·H2O), mobile phase B: ACN; flow rate: 60 mL/min; gradient: 18% B to 37% B in 7 min; wave length: 254 nm/220 nm; RT1(min): 7.43; number of runs: 3) to afford 1-(4-(2-hydroxypropan-2-yl)pyridin-2-yl)-N-(6-methoxy-1-methyl-1H-pyrazolo[4,3-c]pyridin-7-yl)-1H-pyrazole-4-sulfonamide (I-260, 13.2 mg, 9.18%) as a pink solid. LCMS (ES, m/z): [M+1]>=444; ¹H NMR: 1H NMR (400 MHz, DMSO-d₆) δ 1.47 (s, 6H), 3.44 (s, 3H), 4.23 (s, 3H), 5.49 (s, 1H), 7.49 (d, J=5.2 Hz, 1H), 7.62 (s, 1H), 8.08 (s, 1H), 8.17 (s, 1H), 8.42 (d, J=5.2 Hz, 1H), 8.56 (s, 1H), 8.68 (s, 1H).

### Example 335—Synthesis of N-(6-Methoxy-1-Methyl-1H-Pyrazolo[4,3-C]Pyridin-7-yl)-1-(4-(2-Methoxypropan-2-yl)Pyridin-2-yl)-1H-Pyrazole-4-Sulfonamide (I-261)

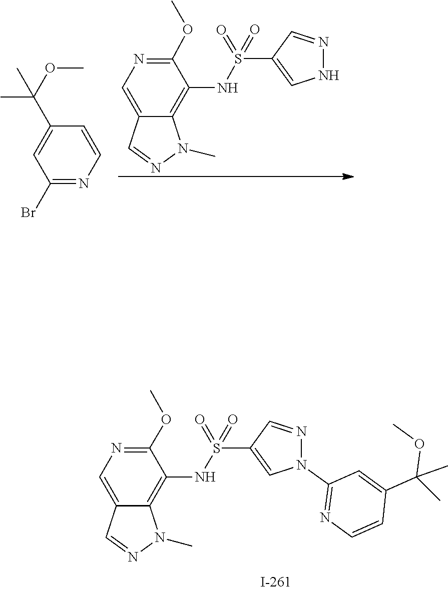

A solution of 2-bromo-4-(2-methoxypropan-2-yl)pyridine (100 mg, 0.435 mmol, 1 equiv), N-{6-methoxy-1-methylpyrazolo[4,3-c]pyridin-7-yl}-1H-pyrazole-4-sulfonamide (200.99 mg, 0.652 mmol, 1.5 equiv), Gphos (23.41 mg, 0.044 mmol, 0.1 equiv), Gphos Pd G6 TES (41.04 mg, 0.044 mmol, 0.1 equiv) and Cs₂CO₃ (424.79 mg, 1.305 mmol, 3 equiv) in DMF (1.5 mL) was stirred for 16 h at 140° C. under a nitrogen atmosphere. The resulting mixture was diluted with water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in N-{6-methoxy-1-methylpyrazolo[4,3-c]pyridin-7-yl}-1-[4-(2-methoxypropan-2-yl) pyridin-2-yl]pyrazole-4-sulfonamide (I-261, 56.7 mg, 28.23%) as a white solid. LCMS (ES, m/z): [M+1]⁺=458; ¹H NMR: (400 MHz, DMSO-d₆) δ 1.50 (s, 6H), 3.11 (s, 3H), 3.45 (s, 3H), 4.23 (s, 3H), 7.48 (dd, J=1.6, 5.2 Hz, 1H), 7.96 (d, J=1.6 Hz, 1H), 8.03 (s, 1H), 8.23 (s, 1H), 8.50 (d, J=5.2 Hz, 1H), 8.66 (s, 1H), 8.75 (s, 1H), 9.94 (s, 1H).

### Example 336—Synthesis of N-(6-Methoxy-1-Methyl-1H-Indazol-7-yl)-1-(4-(OXETAN-3-yl)Pyridin-2-yl)-1H-Pyrazole-4-Sulfonamide (I-262)

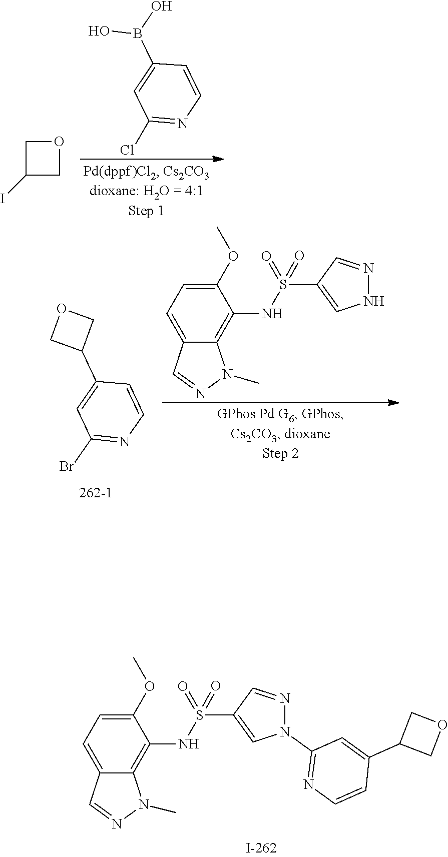

Step 1: Synthesis of 262-1: A solution/mixture of 3-iodooxetane (3 g, 16.306 mmol, 1 equiv), 2-chloropyridin-4-ylboronic acid (6.41 g, 40.766 mmol, 1.5 equiv), Pd(dppf)Cl₂ (1.99 g, 2.718 mmol, 0.1 equiv) and Cs₂CO₃ (15.94 g, 48.918 mmol, 3 equiv) in dioxane (10 mL) and H₂O (5 mL) was stirred overnight at 80° C. under a nitrogen atmosphere. The reaction was quenched with water at room temperature. The resulting mixture was extracted with EtOAc (2×50 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 262-1 (600 mg, 13.02%) as a yellow oil.

Step 2: Synthesis of N-(6-methoxy-1-methyl-1H-indazol-7-yl)-1-(4-(oxetan-3-yl)pyridin-2-yl)-1H-pyrazole-4-sulfonamide (I-262): A solution of 2-chloro-4-(oxetan-3-yl)pyridine (300 mg, 1.769 mmol, 1 equiv), N-(6-methoxy-1-methylindazol-7-yl)-1H-pyrazole-4-sulfonamide (652.31 mg, 2.123 mmol, 1.2 equiv), [3-(tert-butoxy)-6-methoxy-2′,6′-bis(propan-2-yl)-[1,1′-biphenyl]-2-yl]dicyclohexylphosphane (94.94 mg, 0.177 mmol, 0.1 equiv), Gphos Pd G6 TES (167.05 mg, 0.177 mmol, 0.1 equiv) and Cs₂CO₃ (1728.89 mg, 5.307 mmol, 3 equiv) in dioxane (5 mL) was stirred overnight at 80° C. under a nitrogen atmosphere. The residue was dissolved in water (10 mL). The resulting mixture was extracted with EtOAc (2×20 mL). The combined organic layers were washed with brine (2×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in N-(6-methoxy-1-methylindazol-7-yl)-1-[4-(oxetan-3-yl)pyridin-2-yl]pyrazole-4-sulfonamide (I-262, 12.3 mg, 1.58%) as an off-white solid. LCMS (ES, m/z): [M+H]⁺=441; ¹H NMR: (400 MHz, DMSO-d₆): δ 3.33 (s, 3H), 4.24 (s, 3H), 4.36-4.48 (m, 1H), 4.66 (t, J=6.4 Hz, 2H), 5.00 (dd, J=8.4, 6.0 Hz, 2H), 6.88 (d, J=8.8 Hz, 1H), 7.54 (dd, J=5.0, 1.6 Hz, 1H), 7.68 (d, J=8.8 Hz, 1H), 7.96-8.02 (m, 3H), 8.51 (d, J=5.0 Hz, 1H), 8.71 (s, 1H), 9.80 (s, 1H).

### Example 337—Synthesis of N-(6-Methoxy-1-Methyl-1H-Indazol-7-yl)-1-(4-(OXETAN-2-yl)Pyridin-2-yl)-1H-Pyrazole-4-Sulfonamide (I-263)

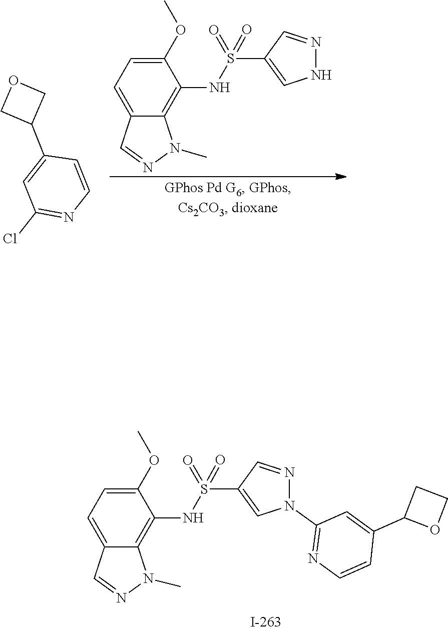

A solution of 2-chloro-4-(oxetan-3-yl)pyridine (300 mg, 1.769 mmol, 1 equiv), N-(6-methoxy-1-methylindazol-7-yl)-1H-pyrazole-4-sulfonamide (652.31 mg, 2.123 mmol, 1.2 equiv), Cs₂CO₃ (1728.89 mg, 5.307 mmol, 3 equiv), [3-(tert-butoxy)-6-methoxy-2′,6′-bis(propan-2-yl)-[1,1′-biphenyl]-2-yl]dicyclohexylphosphane (94.94 mg, 0.177 mmol, 0.1 equiv) and Gphos Pd G6 TES (167.05 mg, 0.177 mmol, 0.1 equiv) in dioxane (5 mL, 59.020 mmol) was stirred overnight at 80° C. under a nitrogen atmosphere. The residue was dissolved in water (10 mL). The resulting mixture was extracted with EtOAc (2×20 mL). The combined organic layers were washed with brine (2×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The product N-(6-methoxy-1-methyl-1H-indazol-7-yl)-1-(4-(oxetan-2-yl)pyridin-2-yl)-1H-pyrazole-4-sulfonamide (I-263,) was isolated. LCMS (ES, m/z): [M+H]=441; ¹H NMR: (400 MHz, DMSO-d₆): δ 2.51-2.62 (m, 1H), 3.06-3.22 (m, 1H), 3.34 (s, 3H), 4.25 (s, 3H), 4.62 (dt, J=9.2, 6.0 Hz, 1H), 4.75 (td, J=8.0, 5.8 Hz, 1H), 5.89 (t, J=7.6 Hz, 1H), 6.88 (d, J=8.8 Hz, 1H), 7.46 (dd, J=5.0, 1.6 Hz, 1H), 7.67 (d, J=8.8 Hz, 1H), 7.99 (m, J=6.6 Hz, 3H), 8.52 (d, J=5.0 Hz, 1H), 8.72 (s, 1H), 9.79 (s, 1H).

### Example 338—Synthesis of N-(6-Methoxy-1-Methyl-1H-Indazol-7-yl)-1′-Methyl-5′-(Trifluoromethyl)-1′ H-[1,3′-Bipyrazole]-4-Sulfonamide (I-266)

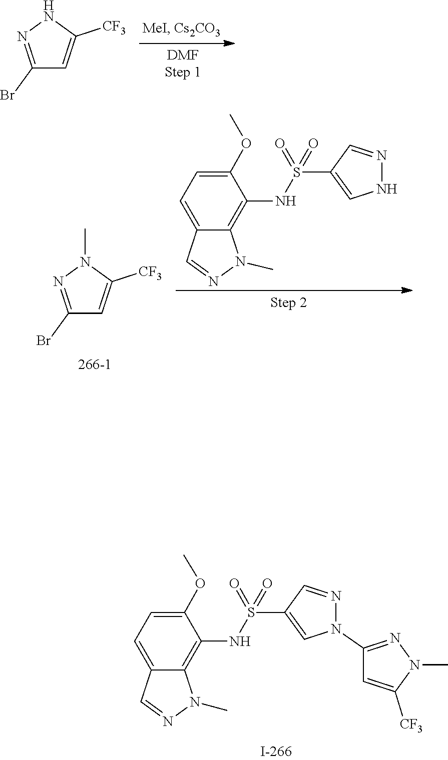

Step 1: Synthesis of 266-1: A solution of 3-bromo-5-(trifluoromethyl)-1H-pyrazole (500 mg, 2.326 mmol, 1 equiv), Cs₂CO₃ (1.52 g, 4.652 mmol, 2 equiv) and Mel (0.43 mL, 6.978 mmol, 3 equiv) in MeCN (6 mL) was stirred for 3 h at room temperature under a nitrogen atmosphere. The resulting mixture was diluted with water (10 mL) and extracted with CH₂Cl₂ (3×10 mL). The combined organic layers were washed with brine (1×30 mL) and dried over anhydrous Na₂SO₄. After filtration, the filtrate was concentrated under reduced pressure. This resulted in 3-bromo-1-methyl-5-(trifluoromethyl)pyrazole (266-1, 480 mg, 63.08%) as a yellow oil. The crude product mixture was used in the next step directly without further purification.

Step 2: Synthesis of N-(6-methoxy-1-methyl-1H-indazol-7-yl)-1′-methyl-5′-(trifluoromethyl)-1′H-[1,3′-bipyrazole]-4-sulfonamide (I-266): To a stirred solution of N-(6-methoxy-1-methylindazol-7-yl)-1H-pyrazole-4-sulfonamide (120 mg, 0.390 mmol, 1.00 equiv), Cs₂CO₃ (381.66 mg, 1.170 mmol, 3 equiv) and 3-bromo-1-methyl-5-(trifluoromethyl)pyrazole (447.08 mg, 0.585 mmol, 1.5 equiv, 30%) in DMF (4 mL) were added CuI (37.18 mg, 0.195 mmol, 0.5 equiv) and N1,N2-dimethylcyclohexane-1,2-diamine (55.54 mg, 0.390 mmol, 1 equiv) at room temperature under a nitrogen atmosphere. The resulting mixture was stirred overnight at 140° C. The resulting mixture was diluted with water (10 mL) and extracted with EtOAc (3×10 mL). The combined organic layers were washed with brine (1×30 mL) and dried over anhydrous Na₂SO₄. After filtration, the filtrate was concentrated under reduced pressure. The crude product (50 mg) was purified by Prep-HPLC with the following conditions (column: XBridge Prep OBD C18 column, 19*250 mm, 5 μm; mobile phase A: water (10 mmol/L NH₄HCO₃+0.05% NH₃H₂O), mobile phase B: MeOH; flow rate: 25 mL/min; gradient: 49% B to 66% B in 10 min; wave length: 254 nm/220 nm; RT1(min): 8.68/9.7; number of runs: 9) to afford N-(6-methoxy-1-methylindazol-7-yl)-1′-methyl-5′-(trifluoromethyl)-[1,3′-bipyrazole]-4-sulfonamide (I-266, 12.3 mg, 6.88%) as an off-white solid. LCMS (ES, m/z): [M+1]+=456; ¹H NMR: ¹H NMR (400 MHz, methanol-d₄) δ 3.42 (s, 3H), 4.01 (s, 3H), 4.33 (s, 3H), 6.85 (d, J=8.8 Hz, 1H), 7.03 (s, 1H), 7.66 (d, J=8.8 Hz, 1H), 7.81 (s, 1H), 7.93 (s, 1H), 8.37 (s, 1H).

### Example 339—Synthesis of 1-(4-((1s,3s)-1-Hydroxy-3-Methyl-Cyclobutyl)Pyridin-2-yl)-N-(1-Methyl-1H-Indazol-7-yl)-1H-Pyrazole-4-Sulfonamide (I-271)

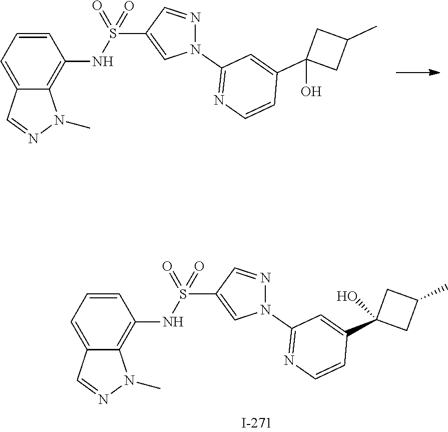 [SMILES: CC1CC(O)(c2ccnc(-n3cc(S(=O)(=O)Nc4cccc5cnn(C)c45)cn3)c2)C1 ; Cn1ncc2cccc(NS(=O)(=O)c3cnn(-c4cc([C@]5(O)C[C@@H](C)C5)ccn4)c3)c21]

1-[4-(1-hydroxy-3-methylcyclobutyl)pyridin-2-yl]-N-(1-methylindazol-7-yl)pyrazole-4-sulfonamide (310 mg, 0.707 mmol, 1 equiv) was purified by Prep-SFC with the following conditions (column: CHIRALPAK IG 4.6*50 mm, 3 μm; mobile phase: MeOH (0.5% 2M NH₃-MeOH), flow rate: 40 mL/min; gradient: 10% to 40% in 4.0 min, hold 2.0 min at 40%; wave length: 254 nm/220 nm; RT1(min): 10.52) to afford 1-(4-((1s,3s)-1-hydroxy-3-methylcyclobutyl)pyridin-2-yl)-N-(1-methyl-1H-indazol-7-yl)-1H-pyrazole-4-sulfonamide (I-271, 243.6 mg, 78.10%) as an off-white solid. LCMS (ES, m/z): [M+1]⁺=439; ¹H NMR: (400 MHz, DMSO-d₆) δ 1.18 (d, J=6.4 Hz, 1H), 2.02-2.07 (m, 1H), 2.12-2.22 (m, 1H), 2.56-2.61 (m, 2H), 4.30 (s, 3H), 6.01 (s, 1H), 6.72 (d, J=7.6 Hz, 1H), 6.96 (t, J=7.6 Hz, 1H), 7.64 (d, J=8.0 Hz, 1H), 7.56-7.57 (m, 1H), 7.64 (d, J=8.0 Hz, 1H), 8.03 (t, J=13.2 Hz, 3H), 8.46 (d, J=5.2 Hz, 1H), 8.78 (s, 1H), 10.02 (s, 1H).

### Example 340—Synthesis of N-(6-Methoxy-1-Methyl-1H-Pyrazolo[4,3-C]Pyridin-7-yl)-1-(4-(1-Methoxyethyl)Pyridin-2-yl)-1H-Pyrazole-4-Sulfonamide (I-272)

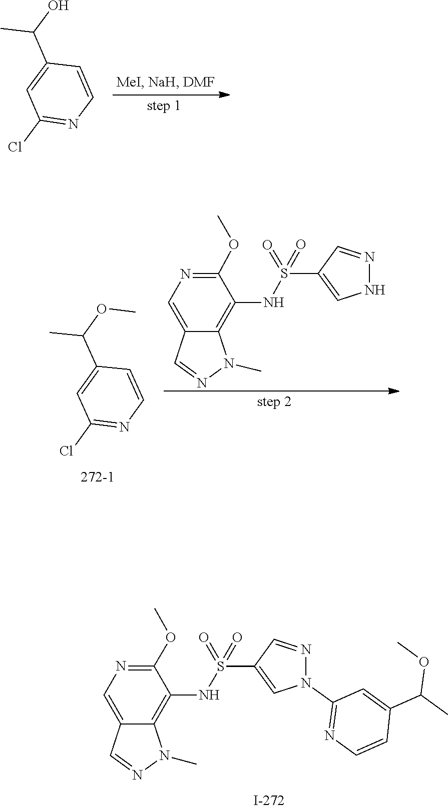

Step 1: Synthesis of 272-1: A solution of 1-(2-chloropyridin-4-yl)ethanol (500 mg, 3.173 mmol, 1 equiv) in DMF (6 mL) was treated with NaH (228.41 mg, 9.519 mmol, 3 equiv) for 30 min at 0° C. under a nitrogen atmosphere followed by the addition of Mel (0.40 mL, 6.346 mmol, 2 equiv) dropwise at room temperature. The resulting mixture was stirred for an additional 3 h at room temperature. The reaction was quenched by the addition of water (10 mL) at 0° C. The resulting mixture was extracted with CH₂Cl₂ (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 0% to 100% gradient in 25 min; detector: UV 254 nm. This resulted in 2-chloro-4-(1-methoxyethyl)pyridine (272-1, 500 mg, 73.46%) as a yellow oil.

Step 2: Synthesis of N-(6-methoxy-1-methyl-1H-pyrazolo[4,3-c]pyridin-7-yl)-1-(4-(1-methoxyethyl)pyridin-2-yl)-1H-pyrazole-4-sulfonamide (I-272): A solution of N-{6-methoxy-1-methylpyrazolo[4,3-c]pyridin-7-yl}-1H-pyrazole-4-sulfonamide (200 mg, 0.649 mmol, 1.00 equiv), Cs₂CO₃ (634.05 mg, 1.947 mmol, 3 equiv) and 2-chloro-4-(1-methoxyethyl)pyridine (166.99 mg, 0.974 mmol, 1.5 equiv) in DMF (6 mL) was stirred overnight at 140° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 20 min; detector: UV 254 nm. The crude product (100 mg) was purified by PREP-CHIRAL-HPLC with the following conditions (column: Lux3umCellulose3; mobile phase A: hex(0.2% TFA): EtOH=80:20; flow rate: 1 mL/min; gradient: isocratic; injection volume: 3 mL) to afford N-{6-methoxy-1-methylpyrazolo[4,3-c]pyridin-7-yl}-1-{4-[1-methoxyethyl]pyridin-2-yl}pyrazole-4-sulfonamide as a single steroisomer (I-272, 22.2 mg, 7.70%) as an off-white solid. LCMS (ES, m/z): [M+1]+=444; ¹H NMR: ¹H NMR (400 MHz, DMSO-d₆) δ 1.38 (d, J=6.8 Hz, 3H), 3.24 (s, 3H), 3.44 (s, 3H), 4.22 (s, 3H), 4.53 (q, J=6.4 Hz, 1H), 7.41 (dd, J=1.6, 5.2 Hz, 1H), 7.92 (s, 1H), 8.02 (s, 1H), 8.23 (s, 1H), 8.50 (d, J=5.2 Hz, 1H), 8.66 (s, 1H), 8.74 (s, 1H), 9.93 (s, 1H).

### Example 341—Synthesis of N-(6-Methoxy-1-Methyl-1H-Pyrazolo[4,3-C]Pyridin-7-yl)-1-(4-(1-Methoxyethyl)Pyridin-2-yl)-1H-Pyrazole-4-Sulfonamide (I-273)

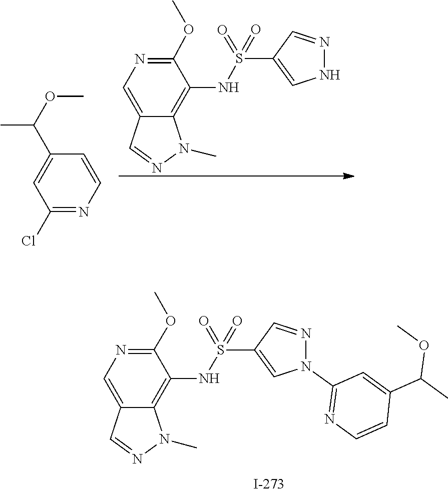

A solution of N-{6-methoxy-1-methylpyrazolo[4,3-c]pyridin-7-yl}-1H-pyrazole-4-sulfonamide (200 mg, 0.649 mmol, 1.00 equiv), Cs₂CO₃ (634.05 mg, 1.947 mmol, 3 equiv) and 2-chloro-4-(1-methoxyethyl)pyridine (166.99 mg, 0.974 mmol, 1.5 equiv) in DMF (6 mL) was stirred overnight at 140° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 20 min; detector: UV 254 nm. The crude product (100 mg) was purified by PREP-CHIRAL-HPLC with the following conditions (column: Lux3umCellulose3; mobile phase A: hex(0.2% TFA): EtOH=80:20; flow rate: 1 mL/min; gradient: isocratic; injection volume: 3 mL) to afford N-{6-methoxy-1-methylpyrazolo[4,3-c]pyridin-7-yl}-1-{4-[1-methoxyethyl]pyridin-2-yl}pyrazole-4-sulfonamide as a single stereoisomer (I-273, 23.1 mg, 7.87%) as an off-white solid. LCMS (ES, m/z): [M+1]⁺=444; ¹H NMR (400 MHz, DMSO-d₆) δ 1.38 (d, J=6.4 Hz, 3H), 3.24 (s, 3H), 3.44 (s, 3H), 4.22 (s, 3H), 4.53 (q, J=6.4 Hz, 1H), 7.42 (d, J=5.2 Hz, 1H), 7.92 (s, 1H), 8.03 (s, 1H), 8.23 (s, 1H), 8.50 (d, J=5.2 Hz, 1H), 8.67 (s, 1H), 8.74 (s, 1H), 9.94 (s, 1H).

### Example 342—Synthesis of N-(6-Ethyl-1-Methyl-1H-Indazol-7-yl)-1-(4-(2-Hydroxypropan-2-yl)Pyridin-2-yl)-1H-Pyrazole-4-Sulfonamide (I-274)

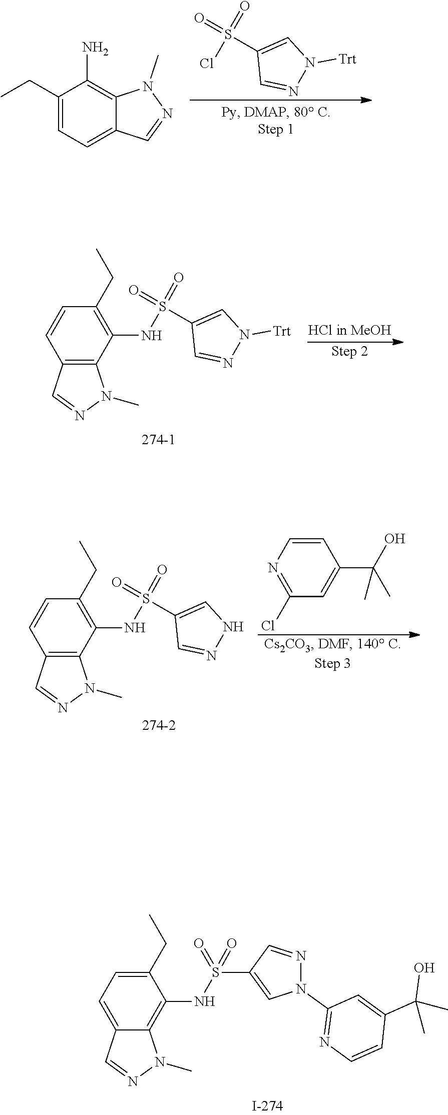

Step 1: Synthesis of 274-1: A solution of 6-ethyl-1-methylindazol-7-amine (1 g, 5.707 mmol, 1 equiv), 1-(triphenylmethyl)pyrazole-4-sulfonyl chloride (2566.78 mg, 6.278 mmol, 1.1 equiv) and DMAP (69.72 mg, 0.571 mmol, 0.1 equiv) in pyridine (10 mL) was stirred overnight at 80° C. under a nitrogen atmosphere. The resulting mixture was concentrated under reduced pressure. The residue was dissolved in MeCN (10 mL). The precipitated solids were collected by filtration and washed with MeCN (3×10 mL). This resulted in N-(6-ethyl-1-methylindazol-7-yl)-1-(triphenylmethyl)pyrazole-4-sulfonamide (274-1, 1.8 g, 57.59%) as a brown solid.

Step 2: Synthesis of 274-2: A solution of N-(6-ethyl-1-methylindazol-7-yl)-1-(triphenylmethyl)pyrazole-4-sulfonamide (1.8 g, 3.287 mmol, 1 equiv) in HCl(g) in MeOH (20 mL) was stirred for 4 h at room temperature under a nitrogen atmosphere. The resulting mixture was concentrated under reduced pressure. The residue was suspended in MeCN (10 mL). The precipitated solids were collected by filtration and washed with MeCN (1×10 mL). This resulted in N-(6-ethyl-1-methylindazol-7-yl)-1H-pyrazole-4-sulfonamide (274-2, 800 mg, 79.71%) as a yellow green solid.

Step 3: Synthesis of N-(6-ethyl-1-methyl-1H-indazol-7-yl)-1-(4-(2-hydroxypropan-2-yl)pyridin-2-yl)-1H-pyrazole-4-sulfonamide (I-274): A solution of N-(6-ethyl-1-methylindazol-7-yl)-1H-pyrazole-4-sulfonamide (213.51 mg, 0.700 mmol, 1.2 equiv) 2-(2-chloropyridin-4-yl)propan-2-ol (100 mg, 0.583 mmol, 1.00 equiv) and Cs₂CO₃ (569.55 mg, 1.749 mmol, 3 equiv) in DMF (5 mL) was stirred overnight at 80° C. under a nitrogen atmosphere. The residue was dissolved in water (10 mL). The resulting mixture was extracted with EtOAc (2×10 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-(6-ethyl-1-methylindazol-7-yl)-1-[4-(2-hydroxypropan-2-yl)pyridin-2-yl]pyrazole-4-sulfonamide (I-274, 59.3 mg, 23.10%) as an off-white solid. LCMS (ES, m/z): [M+H]=441; ¹H NMR: (400 MHz, DMSO-d₆): δ 0.86 (t, J=7.6 Hz, 3H), 1.47 (s, 6H), 2.24 (m, 2H), 4.25 (s, 3H), 5.50 (s, 1H), 6.99 (d, J=8.2 Hz, 1H), 7.52 (dd, J=5.2, 1.6 Hz, 1H), 7.65 (d, J=8.2 Hz, 1H), 8.00 (d, J=2.8 Hz, 2H), 8.09 (d, J=1.6 Hz, 1H), 8.42 (d, J=5.2 Hz, 1H), 8.70 (s, 1H), 10.09 (s, 1H).

### Example 343—Synthesis of 1-(4-(3-HYDROXYPENTAN-3-yl)Pyridin-2-yl)-N-(6-Methoxy-1-Methyl-1H-Indazol-7-yl)-1H-Pyrazole-4-Sulfonamide (I-279)

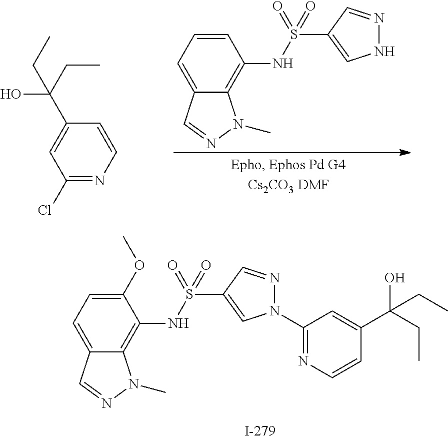 [SMILES: CCC(O)(CC)c1ccnc(Cl)c1 ; Cn1ncc2cccc(NS(=O)(=O)c3cn[nH]c3)c21 ; CCC(O)(CC)c1ccnc(-n2cc(S(=O)(=O)Nc3c(OC)ccc4cnn(C)c34)cn2)c1]

To a stirred mixture of 3-(2-chloropyridin-4-yl)pentan-3-ol (150 mg, 0.751 mmol, 1 equiv), N-(6-methoxy-1-methylindazol-7-yl)-1H-pyrazole-4-sulfonamide (253.95 mg, 0.826 mmol, 1.1 equiv) and Cs₂CO₃ (734.27 mg, 2.253 mmol, 3 equiv) in DMF (4 mL) was added EPhos (40.17 mg, 0.075 mmol, 0.1 equiv) and EPhos Pd G4 (69.00 mg, 0.075 mmol, 0.1 equiv) at r.t. under an N₂ atmosphere. The resulting mixture was stirred at 140° C. overnight under an N₂ atmosphere. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (200 mg) was purified by Prep-HPLC with the following conditions (column: XBridge Prep OBD C18 column, 19*250 mm, 5 μm; mobile phase A: water (10 mmol/L NH₄HCO₃+0.05% NH₃H₂O), mobile phase B: MEOH; flow rate: 25 mL/min; gradient: 49% B to 67% B in 10 min; wave length: 254 nm/220 nm; RT1(min): 9.32; number of runs: 12) to afford 1-[4-(3-hydroxypentan-3-yl)pyridin-2-yl]-N-(6-methoxy-1-methylindazol-7-yl)pyrazole-4-sulfonamide (I-279. 82.7 mg, 23.33%) as a white solid. LCMS (ES, m/z): [M+H]⁺=471; ¹H NMR: ¹H NMR (400 MHz, CD3OD) δ 0.77 (t, J=7.2 Hz, 6H), 1.78-1.95 (m, 4H), 3.39 (s, 3H), 4.34 (s, 3H), 6.83 (d, J=8.8 Hz, 1H), 7.38 (dd, J=1.6, 5.2 Hz, 1H), 7.65 (d, J=8.8 Hz, 1H), 7.83 (s, 1H), 7.93 (s, 1H), 8.07 (d, J=1.6 Hz, 1H), 8.37 (d, J=5.2 Hz, 1H), 8.70-8.79 (m, 1H).

### Example 344—Synthesis of 1-(4-(1-Hydroxypropyl)Pyridin-2-yl)-N-(6-Methoxy-1-Methyl-1H-Indazol-7-yl)-1H-Pyrazole-4-Sulfonamide (I-280)

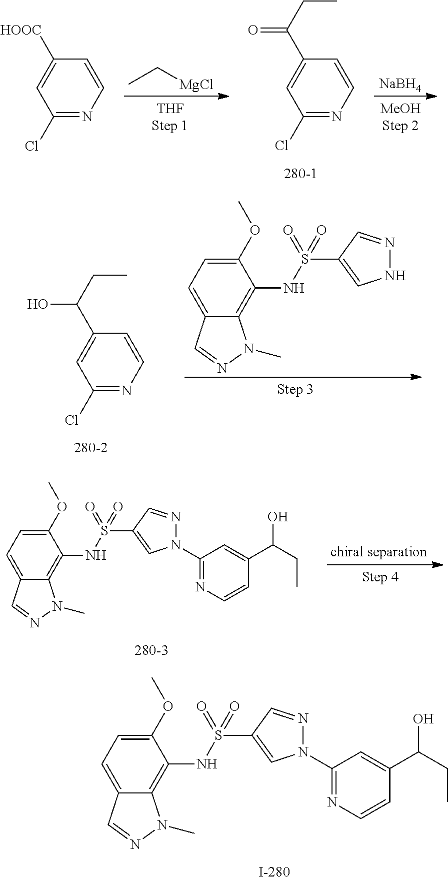

Step 1: Synthesis of 280-1: A slurry of 2-chloropyridin-4-yl formate (5 g, 31.736 mmol, 1 equiv) in THE (20 mL) was cooled to −20° C. and ethylmagnesium chloride (44.5 mL, 529.160 mmol, 2.0 M, 2.8 equiv) in THE solution was added at a rate to keep the internal temperature at <5° C. The reaction mixture was then warmed to 0° C. and stirred for 4 hours. The reaction was quenched with 80 mL sat. NH₄Cl (aq.) at 0° C. The resulting mixture was extracted with CH₂Cl₂ (3×100 ml). The combined organic layers were washed with brine (1×200 mL) and dried over anhydrous Na₂SO₄. After filtration, the filtrate was concentrated under reduced pressure. The residue was purified by silica gel column chromatography and eluted with PE/EA (10:1) to afford 280-1 (1.5 g, 22.29%) as a yellow oil.

Step 2: Synthesis of 280-2: A solution 280-1 (1.5 g, 8.844 mmol, 1 equiv) and NaBH₄ (1.34 g, 35.376 mmol, 4 equiv) in MeOH (10 mL) was stirred for 3 hours at room temperature under a nitrogen atmosphere. The reaction was quenched with water/ice (20 mL) at 0° C. The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. This resulted in 280-2 (1.5 g, crude) as a yellow oil.

Step 3: Synthesis of 280-3: A solution of 280-2 (100 mg, 0.583 mmol, 1 equiv), N-(6-methoxy-1-methylindazol-7-yl)-1H-pyrazole-4-sulfonamide (214.89 mg, 0.700 mmol, 1.2 equiv), EPhos (31.16 mg, 0.058 mmol, 0.1 equiv), EPhos Pd G4 (53.52 mg, 0.058 mmol, 0.1 equiv) and Cs₂CO₃ (569.55 mg, 1.749 mmol, 3 equiv) in DMF (3 mL) was stirred for 4 hours at 140° C. under a nitrogen atmosphere. The residue was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 40 min; detector: UV 254 nm. This resulted in 280-3 (120 mg, crude) as an off-white solid.

Step 4: Synthesis of 1-(4-(1-hydroxypropyl)pyridin-2-yl)-N-(6-methoxy-1-methyl-1H-indazol-7-yl)-1H-pyrazole-4-sulfonamide (I-280): The crude product 280-3 (120 mg) was purified by chiral HPLC with the following conditions (column: Lux3umCellulose4; mobile phase A: hex(0.2% TFA): EtOH=70:30; flow rate: 1 mL/min; gradient: isocratic; injection volume: 3 L) to afford 1-(4-(1-hydroxypropyl)pyridin-2-yl)-N-(6-methoxy-1-methyl-1H-indazol-7-yl)-1H-pyrazole-4-sulfonamide as a single steroisomer (I-280, 39.1 mg, 14.97%) as an off-white solid. LCMS (ES, m/z): [M+H]⁺=442; ¹H NMR: (400 MHz, methanol-d₄) δ 0.97 (t, J=7.2 Hz, 3H), 1.67-1.88 (m, 2H), 3.39 (s, 3H), 4.34 (s, 3H), 4.68 (dd, J=5.2, 7.6 Hz, 1H), 6.84 (d, J=8.8 Hz, 1H), 7.35 (dd, J=1.6, 5.2 Hz, 1H), 7.66 (d, J=8.8 Hz, 1H), 7.83 (s, 1H), 7.93 (s, 1H), 8.01 (d, J=1.2 Hz, 1H), 8.38 (d, J=8.0 Hz, 1H), 8.72 (s, 1H).

### Example 345—Synthesis of 1-(4-(1-Hydroxypropyl)Pyridin-2-yl)-N-(6-Methoxy-1-Methyl-1H-Indazol-7-yl)-1H-Pyrazole-4-Sulfonamide (I-281)

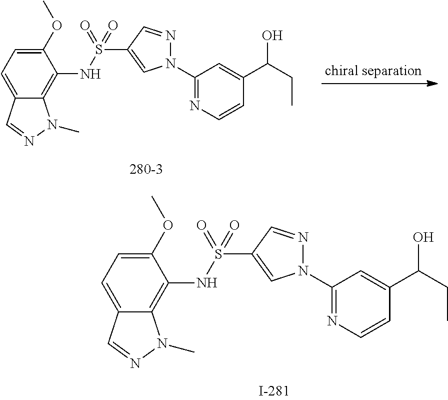 [SMILES: CCC(O)c1ccnc(-n2cc(S(=O)(=O)Nc3c(OC)ccc4cnn(C)c34)cn2)c1 ; CCC(O)c1ccnc(-n2cc(S(=O)(=O)Nc3c(OC)ccc4cnn(C)c34)cn2)c1]

The crude product 280-3 (120 mg) was purified by chiral HPLC with the following conditions (column: Lux 3 μm Cellulose-4; mobile phase A: hex(0.2% TFA): EtOH=70:30; flow rate: 1 mL/min; gradient: isocratic; injection volume: 3 L) to afford 1-(4-(1-hydroxypropyl)pyridin-2-yl)-N-(6-methoxy-1-methyl-1H-indazol-7-yl)-1H-pyrazole-4-sulfonamide as a single stereoisomer (I-281, 37.8 mg, 14.38%) as an off-white solid. LCMS (ES, m/z): [M+H]⁺=442; ¹H NMR: (400 MHz, methanol-d₄) δ 0.97 (t, J=7.2 Hz, 3H), 1.67-1.88 (m, 2H), 3.39 (s, 3H), 4.34 (s, 3H), 4.68 (dd, J=5.2, 7.6 Hz, 1H), 6.84 (d, J=8.8 Hz, 1H), 7.35 (dd, J=1.6, 5.2 Hz, 1H), 7.66 (d, J=8.8 Hz, 1H), 7.83 (s, 1H), 7.93 (s, 1H), 8.01 (d, J=1.2 Hz, 1H), 8.38 (d, J=8.0 Hz, 1H), 8.72 (s, 1H).

### Example 346—Synthesis of N-(1-Methyl-1H-Indazol-7-yl)-1-(4-(2-Oxooxazolidin-3-yl)Pyridin-2-yl)-1H-Pyrazole-4-Sulfonamide (I-282)

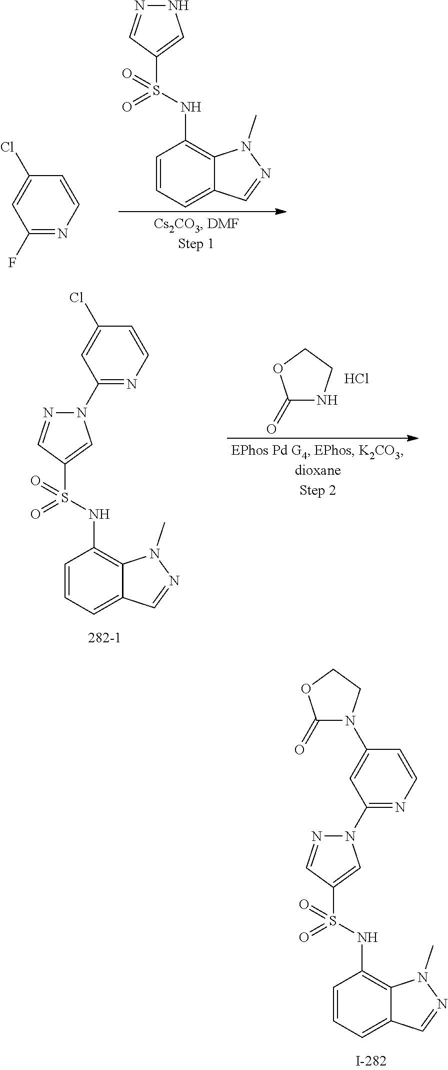 [SMILES: Fc1cc(Cl)ccn1 ; Cn1ncc2cccc(NS(=O)(=O)c3cn[nH]c3)c21 ; Cn1ncc2cccc(NS(=O)(=O)c3cnn(-c4cc(Cl)ccn4)c3)c21 ; O=C1NCCO1 ; Cl ; Cn1ncc2cccc(NS(=O)(=O)c3cnn(-c4cc(N5CCOC5=O)ccn4)c3)c21]

Step 1: Synthesis of 282-1: To a stirred solution of 4-chloro-2-fluoropyridine (0.57 g, 4.327 mmol, 1.2 equiv) and N-(1-methylindazol-7-yl)-1H-pyrazole-4-sulfonamide (1 g, 3.606 mmol, 1.00 equiv) in DMF (10 mL) were added Cs₂CO₃ (3.52 g, 10.818 mmol, 3 equiv) in portions at room temperature under a nitrogen atmosphere. The resulting mixture was stirred for 2 h at 80° C. under a nitrogen atmosphere. The resulting mixture was diluted with water (50 mL). The resulting mixture was extracted with EtOAc (3×60 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 35% to 40% gradient in 12 min; detector: UV 254 nm. The resulting mixture was concentrated under reduced pressure. This resulted in 1-(4-chloropyridin-2-yl)-N-(1-methylindazol-7-yl) pyrazole-4-sulfonamide (282-1, 800 mg, 57.05%) as a pink solid.

Step 2: Synthesis of N-(1-methyl-1H-indazol-7-yl)-1-(4-(2-oxooxazolidin-3-yl)pyridin-2-yl)-1H-pyrazole-4-sulfonamide (I-282): To a stirred mixture of 1-(4-chloropyridin-2-yl)-N-(1-methylindazol-7-yl) pyrazole-4-sulfonamide (80 mg, 0.206 mmol, 1 equiv) and oxazolidinone (26.87 mg, 0.309 mmol, 1.5 equiv) in dioxane (3 mL) were added EPhos (11.00 mg, 0.021 mmol, 0.1 equiv), EPhos Pd G4 (18.90 mg, 0.021 mmol, 0.1 equiv) and K₂CO₃ (85.31 mg, 0.618 mmol, 3 equiv) in portions at room temperature under a nitrogen atmosphere. The resulting mixture was stirred for 3 h at 80° C. under a nitrogen atmosphere. The resulting mixture was diluted with water (10 mL). The resulting mixture was extracted with EtOAc (3×15 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 46% to 55% gradient in 9 min; detector: UV 254 nm. The resulting mixture was concentrated under reduced pressure. This resulted in N-(1-methylindazol-7-yl)-1-[4-(2-oxo-1,3-oxazolidin-3-yl) pyridin-2-yl] pyrazole-4-sulfonamide (I-282, 56.2 mg, 60.85%) as an off-white solid. LCMS (ES, m/z): [M+H]⁺=440; ¹H NMR: (400 MHz, DMSO-d₆) δ 4.12-4.19 (m, 2H), 4.28 (s, 3H), 4.49-4.56 (m, 2H), 6.71 (d, J=8 Hz, 1H), 6.98 (t, J=8 Hz, 1H), 7.51 (dd, J=5.8, 4 Hz, 1H), 7.70 (d, J=8 Hz, 1H), 8.03 (d, J=0.8 Hz, 1H), 8.09 (s, 1H), 8.31 (d, J=2.0 Hz, 1H), 8.44 (d, J=8 Hz, 1H), 8.79 (d, J=0.8 Hz, 1H), 10.04 (s, 1H).

### Example 347—Synthesis of 1-(4-(4,4-Dimethyl-2-Oxoimidazolidin-1-yl)Pyridin-2-yl)-N-(1-Methyl-1H-Indazol-7-yl)-1H-Pyrazole-4-Sulfonamide (I-285)

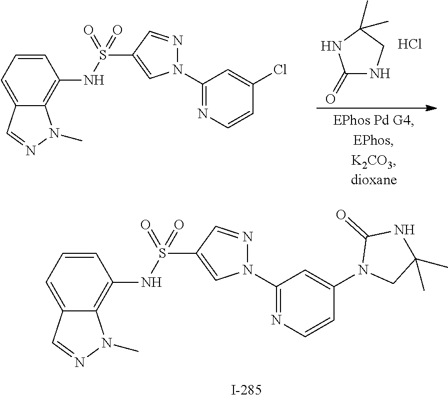

To a stirred solution of 1-(4-chloropyridin-2-yl)-N-(1-methylindazol-7-yl) pyrazole-4-sulfonamide (80 mg, 0.206 mmol, 1 equiv) and 4,4-dimethylimidazolidin-2-one (35.23 mg, 0.309 mmol, 1.5 equiv) in dioxane (3 mL) were added EPhos (11.00 mg, 0.021 mmol, 0.1 equiv), EPhos Pd G4 (18.90 mg, 0.021 mmol, 0.1 equiv) and K₂CO₃ (85.31 mg, 0.618 mmol, 3 equiv) in portions at room temperature under a nitrogen atmosphere. The resulting mixture was stirred for 8 h at 80° C. under a nitrogen atmosphere. The resulting mixture was diluted with water (50 mL). The resulting mixture was extracted with EtOAc (3×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 35% to 40% gradient in 12 min; detector: UV 254 nm. The resulting mixture was concentrated under reduced pressure. This resulted in 1-[4-(4,4-dimethyl-2-oxoimidazolidin-1-yl) pyridin-2-yl]-N-(1-methylindazol-7-yl) pyrazole-4-sulfonamide (I-285, 10.1 mg, 10.50%) as a white solid. LCMS (ES, m/z): [M+H]⁺=467; ¹H NMR: (400 MHz, DMSO-d₆) δ 1.30 (s, 6H), 3.71 (s, 2H), 4.28 (s, 3H), 6.70 (d, J=7.2 Hz, 1H), 6.97 (t, J=8 Hz, 1H), 7.44 (dd, J=2, 2.2 Hz, 1H), 7.68 (d, J=8.0 Hz, 1H), 7.77 (s, 1H), 7.99 (s, 1H), 8.08 (s, 1H), 8.30 (d, J=4 Hz, 2H), 8.75 (s, 1H), 10.00 (s, 1H).

### Example 348—Synthesis of N-(6-Methoxy-1-Methyl-1H-Pyrazolo[4,3-C]Pyridin-7-yl)-1′-Methyl-5′-(Trifluoromethyl)-1′ H-[1,3′-Bipyrazole1-4-Sulfonamide (I-286)

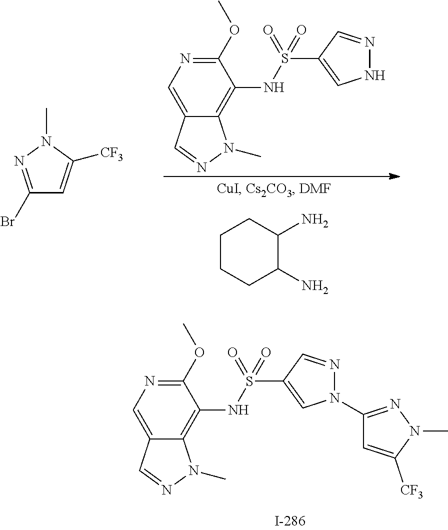 [SMILES: Cn1nc(Br)cc1C(F)(F)F ; COc1ncc2cnn(C)c2c1NS(=O)(=O)c1cn[nH]c1 ; NC1CCCCC1N ; COc1ncc2cnn(C)c2c1NS(=O)(=O)c1cnn(-c2cc(C(F)(F)F)n(C)n2)c1]

A solution of N-{6-methoxy-1-methylpyrazolo[4,3-c]pyridin-7-yl}-1H-pyrazole-4-sulfonamide (160 mg, 0.519 mmol, 1 equiv), 3-bromo-1-methyl-5-(trifluoromethyl)pyrazole (237.68 mg, 1.038 mmol, 2 equiv), 1,2-cyclohexanediamine (59.26 mg, 0.519 mmol, 1 equiv), CuI (49.42 mg, 0.260 mmol, 0.5 equiv) and Cs₂CO₃ (507.24 mg, 1.557 mmol, 3 equiv) in DMF (5 mL) was stirred for 3 h at 140° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 15% B to 40% B in 7 min; wave length: 220 nm; RT1(min): 8.52) to afford N-(6-methoxy-1-methyl-1H-pyrazolo[4,3-c]pyridin-7-yl)-1′-methyl-5′-(trifluoromethyl)-1′H-[1,3′-bipyrazole]-4-sulfonamide (I-286, 8.3 mg, 3.50%) as a white solid. LCMS (ES, m/z): [M+1]+=457; ¹H NMR: ¹H NMR (400 MHz, DMSO-d₆) δ 3.33 (s, 3H), 4.00 (s, 3H), 4.20 (s, 3H), 7.30 (s, 1H), 7.96 (s, 1H), 8.21 (s, 1H), 8.53 (s, 1H), 8.65 (s, 1H), 9.84 (s, 1H).

### Example 349—Synthesis of 1-(4-((1r,3r)-1-Hydroxy-3-Methyl-Cyclobutyl)Pyridin-2-yl)-N-(1-Methyl-1H-Indazol-7-yl)-1H-Pyrazole-4-Sulfonamide (I-288)

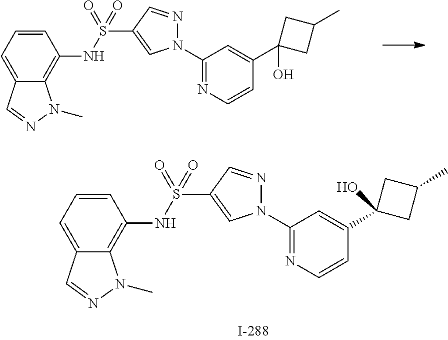 [SMILES: CC1CC(O)(c2ccnc(-n3cc(S(=O)(=O)Nc4cccc5cnn(C)c45)cn3)c2)C1 ; Cn1ncc2cccc(NS(=O)(=O)c3cnn(-c4cc([C@]5(O)C[C@H](C)C5)ccn4)c3)c21]

1-[4-(1-hydroxy-3-methylcyclobutyl)pyridin-2-yl]-N-(1-methylindazol-7-yl)pyrazole-4-sulfonamide (310 mg, 0.707 mmol, 1 equiv) was purified by Prep-SFC with the following conditions (column: CHIRALPAK IG 4.6*50 mm, 3 μm; mobile phase: MeOH (0.5% 2M NH₃-MeOH), flow rate: 40 mL/min; gradient: 10% to 40% in 4.0 min, hold 2.0 min at 40%; wave length: 254 nm/220 nm; RT₁(min): 10.52) to afford 1-(4-((1r,3r)-1-hydroxy-3-methyl-cyclobutyl)pyridin-2-yl)-N-(1-methyl-1H-indazol-7-yl)-1H-pyrazole-4-sulfonamide (I-288, 20.1 mg, 6.46%) as an off-white solid. LCMS (ES, m/z): [M+1]+=439; ¹H NMR: (400 MHz, DMSO-d₆) δ 1.14 (d, J=6.4 Hz, 3H), 2.01-2.06 (m, 2H), 2.36-2.51 (m, 2H), 2.68-2.77 (m, 1H), 4.29 (s, 3H), 6.71 (d, J=7.2 Hz, 1H), 6.98 (t, J=7.6 Hz, 1H), 7.52 (d, J=5.2 Hz, 1H), 7.71 (d, J=8.0 Hz, 1H), 8.03 (s, 2H), 8.10 (s, 1H) 8.46 (d, J=5.2 Hz, 1H), 8.80 (s, 1H), 10.04 (s, 1H).

### Example 350—Synthesis of N-(6-Methoxy-1-Methyl-1H-Indazol-7-yl)-1-(4-(1-Methoxyethyl)Pyridin-2-yl)-1H-Pyrazole-4-Sulfonamide (I-290)

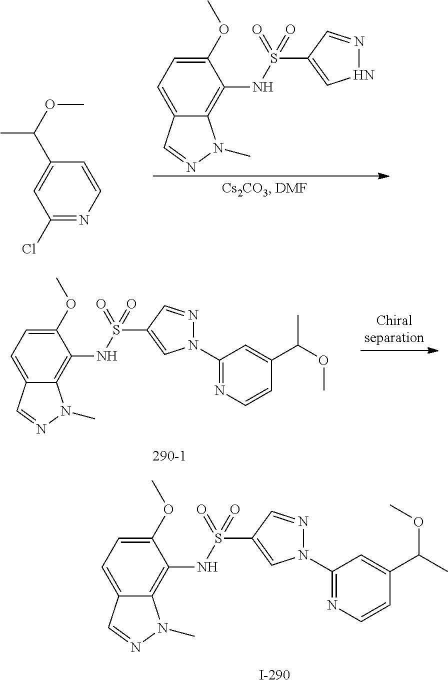

Step 1: Synthesis of 290-1: A solution of 2-chloro-4-(1-methoxyethyl)pyridine (100 mg, 0.583 mmol, 1 equiv), N-(6-methoxy-1-methylindazol-7-yl)-1H-pyrazole-4-sulfonamide (214.89 mg, 0.700 mmol, 1.2 equiv) and Cs₂CO₃ (569.55 mg, 1.749 mmol, 3 equiv) in DMF (5 mL) was stirred overnight at 140° C. under a nitrogen atmosphere. The reaction mixture was dissolved in water (10 mL). The resulting mixture was extracted with EtOAc (2×50 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 290-1 (150 mg, 58.18%) as an off-white solid.

Step 2: Synthesis of N-(6-methoxy-1-methyl-1H-indazol-7-yl)-1-(4-(1-methoxyethyl) pyridin-2-yl)-1H-pyrazole-4-sulfonamide (I-290): 290-1 (150 mg) was purified by Prep-Chiral-HPLC with the following conditions (column: CHIRALPAKIH3; mobile phase A: hex(0.2% TFA): EtOH=75:25; flow rate: 1 mL/min; gradient: isocratic; injection volume: 3 mL; the first peak was product) to afford N-(6-methoxy-1-methyl-1H-indazol-7-yl)-1-(4-(1-methoxyethyl)pyridin-2-yl)-1H-pyrazole-4-sulfonamide as a single stereoisomer (1-290, 68.4 mg, 45.60%) as an off-white solid. LCMS (ES, m/z): [M+H]⁺=443; ¹H NMR: (400 MHz, DMSO-d₆): δ 1.38 (d, J=6.6 Hz, 3H), 3.25 (s, 3H), 3.34 (s, 3H), 4.24 (s, 3H), 4.53 (q, J=6.6 Hz, 1H), 6.88 (d, J=8.8 Hz, 1H), 7.38-7.44 (m, 1H), 7.68 (d, J=8.8 Hz, 1H), 7.92 (s, 1H), 7.99 (d, J=4.2 Hz, 2H), 8.49 (d, J=5.0 Hz, 1H), 8.71 (s, 1H), 9.79 (s, 1H)

### Example 351—Synthesis of N-(6-Methoxy-1-Methyl-1H-Indazol-7-yl)-1-(4-(1-Methoxyethyl)Pyridin-2-yl)-1H-Pyrazole-4-Sulfonamide (I-291)

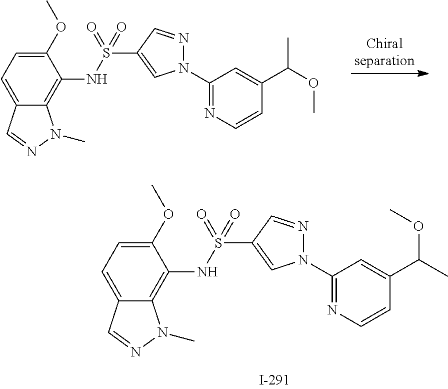 [SMILES: COc1ccc2cnn(C)c2c1NS(=O)(=O)c1cnn(-c2cc(C(C)OC)ccn2)c1 ; COc1ccc2cnn(C)c2c1NS(=O)(=O)c1cnn(-c2cc(C(C)OC)ccn2)c1]

290-1 (150 mg) was purified by Prep-Chiral-HPLC with the following conditions (column: CHIRALPAKIH3; mobile phase A: hex(0.2% TFA): EtOH=75:25; flow rate: 1 mL/min; gradient: isocratic; injection volume: 3 mL; the second peak was product) to afford (R)—N-(6-methoxy-1-methyl-1H-indazol-7-yl)-1-(4-(1-methoxyethyl)pyridin-2-yl)-1H-pyrazole-4-sulfonamide as a single steroeoisomer (1-291, 60.3 mg, 40.20%) as an off-white solid. LCMS (ES, m/z): [M+H]⁺=443; ¹H NMR: (400 MHz, DMSO-d₆): δ 1.38 (d, J=6.6 Hz, 3H), 3.25 (s, 3H), 3.34 (s, 3H), 4.24 (s, 3H), 4.53 (q, J=6.6 Hz, 1H), 6.88 (d, J=8.8 Hz, 1H), 7.38-7.44 (m, 1H), 7.68 (d, J=8.8 Hz, 1H), 7.92 (s, 1H), 7.99 (d, J=4.2 Hz, 2H), 8.49 (d, J=5.0 Hz, 1H), 8.71 (s, 1H), 9.79 (s, 1H)

### Example 352—Synthesis of 1-(4-(1-Hydroxyethyl)Pyridin-2-yl)-N-(6-Methoxy-1-Methyl-1H-Pyrazolo[4,3-C]Pyridin-7-yl)-1H-Pyrazole-4-Sulfonamide (I-292)

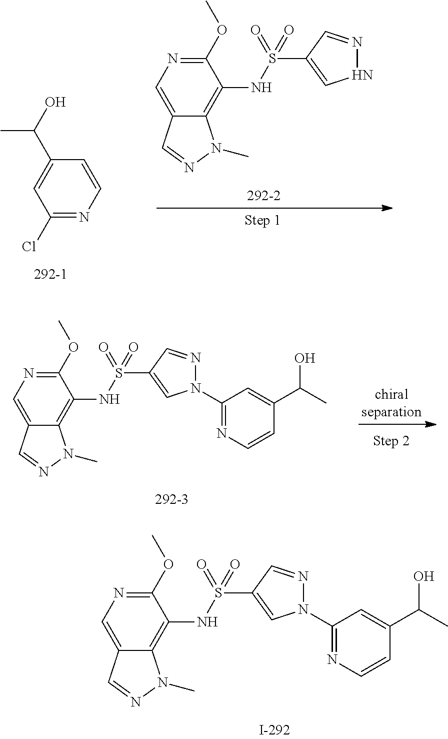

Step 1: Synthesis of 292-3: To a stirred solution of 1-(2-chloropyridin-4-yl)ethanol (102.23 mg, 0.649 mmol, 1 equiv) and N-{6-methoxy-1-methylpyrazolo[4,3-c]pyridin-7-yl}-1H-pyrazole-4-sulfonamide (200 mg, 0.649 mmol, 1.00 equiv) in DMF (2 mL) were added EPhos (34.69 mg, 0.065 mmol, 0.1 equiv), EPhos Pd G4 (59.59 mg, 0.065 mmol, 0.1 equiv) and Cs₂CO₃ (634.05 mg, 1.947 mmol, 3 equiv) at 140° C. under a nitrogen atmosphere. The resulting mixture was stirred overnight at 140° C. under a nitrogen atmosphere. The resulting mixture was diluted with water (10 mL) and extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: MeCN in water (0.1% FA), 0% to 100% gradient in 20 min; detector: UV 254 nm. The resulting mixture was concentrated under vacuum. This resulted in 1-[4-(1-hydroxyethyl)pyridin-2-yl]-N-{6-methoxy-1-methylpyrazolo[4,3-c]pyridin-7-yl}pyrazole-4 sulfonamide (292-3, 54 mg, 8.92%) as a white solid.

Step 2: Synthesis of 1-(4-(1-hydroxyethyl)pyridin-2-yl)-N-(6-methoxy-1-methyl-1H-pyrazolo[4,3-c]pyridin-7-yl)-1H-pyrazole-4-sulfonamide (I-292): The crude product 292-3 (54 mg crude) was purified by chiral HPLC with the following conditions (column: Lux3umCellulose2; mobile phase A: hex(0.2% DEA): EtOH=70:30; flow rate: 1 mL/min; gradient: isocratic; injection volume: 3 L) to afford 1-(4-(1-hydroxyethyl)pyridin-2-yl)-N-(6-methoxy-1-methyl-1H-pyrazolo[4,3-c]pyridin-7-yl)-1H-pyrazole-4-sulfonamide as a single steroeoisomer (I-292, 15.3 mg, 28.25%) as an off-white solid. LCMS (ES, m/z): [M+H]⁺=430; ¹H NMR: (400 MHz, DMSO-d₆) δ 1.38 (d, J=6.4 Hz, 3H), 3.43 (s, 3H), 4.22 (s, 3H), 4.83-4.90 (m, 1H), 5.61 (d, J=4.6 Hz, 1H), 7.42 (d, J=5.2 Hz, 1H), 7.99 (s, 1H), 8.00 (s, 1H), 8.23 (s, 1H), 8.44 (d, J=5.2 Hz, 1H), 8.66 (s, 1H), 8.73 (s, 1H), 9.91 (s, 1H).

### Example 353—Synthesis of 1-(4-(1-Hydroxyethyl)Pyridin-2-yl)-N-(6-Methoxy-1-Methyl-1H-Pyrazolo[4,3-C]Pyridin-7-yl)-1H-Pyrazole-4-Sulfonamide (I-293)

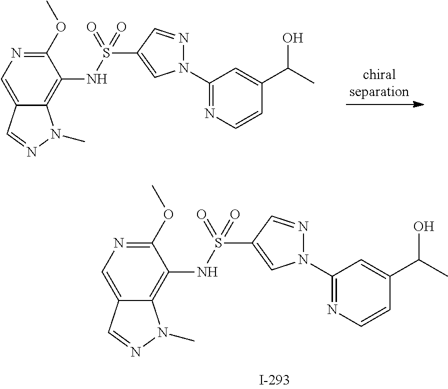 [SMILES: COc1ncc2cnn(C)c2c1NS(=O)(=O)c1cnn(-c2cc(C(C)O)ccn2)c1 ; COc1ncc2cnn(C)c2c1NS(=O)(=O)c1cnn(-c2cc(C(C)O)ccn2)c1]

The crude product 292-3 (54 mg crude) was purified by chiral HPLC with the following conditions (column: Lux3umCellulose2; mobile phase A: hex(0.2% DEA): EtOH=70:30; flow rate: 1 mL/min; gradient: isocratic; injection volume: 3 mL) to afford (R)-1-(4-(1-hydroxyethyl)pyridin-2-yl)-N-(6-methoxy-1-methyl-1H-pyrazolo[4,3-c]pyridin-7-yl)-1H-pyrazole-4-sulfonamide as a single steroeoisomer (1-293, 19.1 mg, 34.49%) as a white solid. LCMS (ES, m/z): [M+H]⁺=430; ¹H NMR: (400 MHz, DMSO-d₆) δ 1.38 (d, J=6.4 Hz, 3H), 3.43 (s, 3H), 4.22 (s, 3H), 4.86 (q, J=6.4 Hz, 1H), 5.61 (s, 1H), 7.42 (d, J=5.2 Hz, 1H), 8.00 (d, J=6.2 Hz, 2H), 8.22 (s, 1H), 8.44 (d, J=5.1 Hz, 1H), 8.66 (s, 1H), 8.73 (s, 1H), 9.91 (s, 1H).

### Example 354—Synthesis of N-(6-Methoxy-1-Methyl-1H-Indazol-7-yl)-1-(4-(3-Methoxypentan-3-yl)Pyridin-2-yl)-1H-Pyrazole-4-Sulfonamide (I-299)

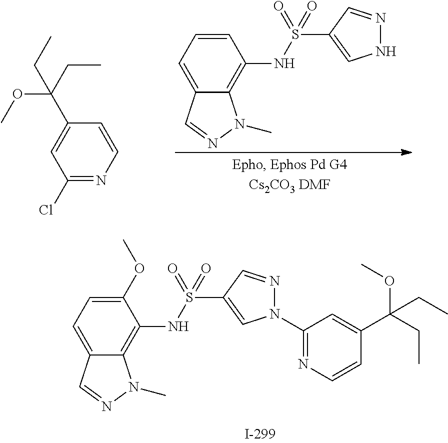 [SMILES: CCC(CC)(OC)c1ccnc(Cl)c1 ; Cn1ncc2cccc(NS(=O)(=O)c3cn[nH]c3)c21 ; CCC(CC)(OC)c1ccnc(-n2cc(S(=O)(=O)Nc3c(OC)ccc4cnn(C)c34)cn2)c1]

To a stirred solution of N-(6-methoxy-1-methylindazol-7-yl)-1H-pyrazole-4-sulfonamide (200 mg, 0.651 mmol, 1 equiv) and 2-chloro-4-(3-methoxypentan-3-yl)pyridine (139.08 mg, 0.651 mmol, 1 equiv) in DMF (4 mL) was added Cs₂CO₃ (636.10 mg, 1.953 mmol, 3 equiv), Ephos (34.80 mg, 0.065 mmol, 0.1 equiv) and Ephos Pd G4 (59.78 mg, 0.065 mmol, 0.1 equiv) at room temperature under a nitrogen atmosphere. The resulting mixture was stirred overnight at 140° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 26% B to 52% B in 7 min; wave length: 220 nm; RT1(min): 8.5) to afford 1-[4-(3-hydroxypentan-3-yl)pyridin-2-yl]-N-(6-methoxy-1-methylindazol-7-yl)pyrazole-4-sulfonamide (1-299, 74.4 mg, 23.91%) as a white solid. LCMS (ES, m/z): [M+1]⁺=485; ¹H NMR:¹H NMR (400 MHz, methanol-d₄) δ 0.57 (t, J=7.2 Hz, 6H), 1.72 (q, J=7.2 Hz, 2H), 1.95 (q, J=7.2 Hz, 2H), 3.13 (s, 3H), 3.30 (s, 3H), 4.33 (s, 3H), 6.84 (d, J=8.8 Hz, 1H), 7.38 (dd, J=1.6, 5.2 Hz, 1H), 7.66 (d, J=8.8 Hz, 1H), 7.84 (s, 1H), 7.93 (s, 1H), 8.03 (s, 1H), 8.38 (d, J=1.6 Hz, 1H), 8.72 (s, 1H).

### Example 355—Synthesis of (R)—N-(1-Methyl-1H-Indazol-7-yl)-1-(1-Methylpiperidin-3-yl)-1H-Pyrazole-4-Sulfonamide (I-308)

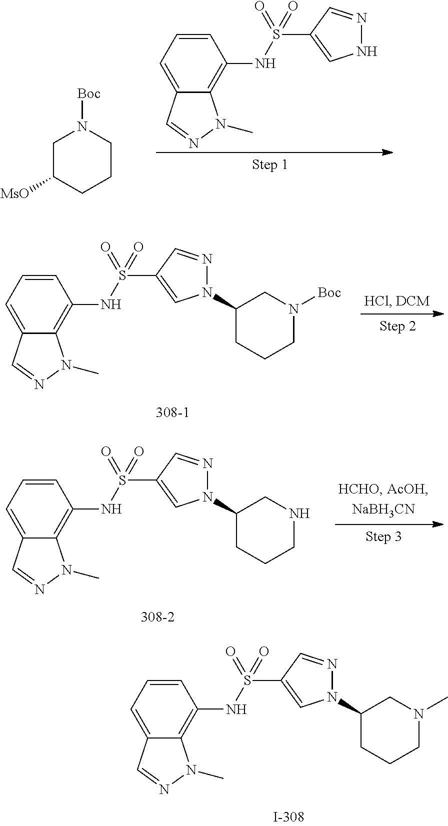 [SMILES: CC(C)(C)OC(=O)N1CCC[C@H](OS(C)(=O)=O)C1 ; Cn1ncc2cccc(NS(=O)(=O)c3cn[nH]c3)c21 ; Cn1ncc2cccc(NS(=O)(=O)c3cnn([C@@H]4CCCN(C(=O)OC(C)(C)C)C4)c3)c21 ; Cn1ncc2cccc(NS(=O)(=O)c3cnn([C@@H]4CCCNC4)c3)c21 ; CN1CCC[C@@H](n2cc(S(=O)(=O)Nc3cccc4cnn(C)c34)cn2)C1]

Step 1: Synthesis of 308-1: A mixture of tert-butyl (3S)-3-(methanesulfonyloxy) piperidine-1-carboxylate (300 mg, 1.07 mmol, 1.00 equiv), N-(1-methylindazol-7-yl)-1H-pyrazole-4-sulfonamide (297 mg, 1.07 mmol, 1.00 equiv) and Cs₂CO₃ (326 mg, 3.22 mmol, 3.00 equiv) in DMF (5 mL) was stirred overnight at 80° C. under a nitrogen atmosphere. The reaction was quenched with water at room temperature. The resulting mixture was extracted with EtOAc (3×5 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in tert-butyl (3R)-3-{4-[(1-methylindazol-7-yl)sulfamoyl]pyrazol-1-yl}piperidine-1-carboxylate (308-1, 140 mg, 28%) as a yellow solid. LCMS (ES, m/z): [M+H]=461

Step 2: Synthesis of 308-2: To a stirred mixture of tert-butyl (3R)-3-{4-[(1-methylindazol-7-yl) sulfamoyl] pyrazol-1-yl} piperidine-1-carboxylate (140 mg, 0.304 mmol, 1.00 equiv) in DCM (4 mL) were added HCl(gas) in 1,4-dioxane (2 mL) dropwise at 0° C. under a nitrogen atmosphere. The resulting mixture was stirred for 1 h at room temperature under a nitrogen atmosphere. The resulting mixture was concentrated under reduced pressure. The crude product (308-2) was used in the next step directly without further purification. LCMS (ES, m/z): [M+H]⁺=361

Step 3: Synthesis of (R)—N-(1-methyl-1H-indazol-7-yl)-1-(1-methylpiperidin-3-yl)-1H-pyrazole-4-sulfonamide (I-308): A mixture of methyl 2-methyl-2-(2-{4-[(1-methylindazol-7-yl) sulfamoyl] pyrazol-1-yl} pyridin-4-yl) propanoate (80 mg, 0.176 mmol, 1.00 equiv), AcOH (66 mg, 1.11 mmol, 5.00 equiv) and formaldehyde (66 mg, 2.22 mmol, 10.0 equiv) in DCM (1.5 mL) and MeOH (1.5 mL) was stirred for 2 h at room temperature under a nitrogen atmosphere. To the above mixture was added NaBH₃CN (41 mg, 0.666 mmol, 3.00 equiv) dropwise at 0° C. The resulting mixture was stirred overnight at room temperature. The reaction was quenched with water at room temperature. The resulting mixture was extracted with CH₂Cl₂ (1×3 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 60% gradient in 10 min; detector: UV 254 nm. This resulted in N-(1-methylindazol-7-yl)-1-[(3R)-1-methylpiperidin-3-yl]pyrazole-4-sulfonamide (I-308, 34.4 mg, 41%) as an off-white solid. LCMS (ES, m/z): [M+H]=375; ¹H NMR: ¹H NMR (400 MHz, methanol-d₄) δ1.62-1.88 (m, 3H), 1.99-2.08 (m, 1H), 2.12 (t, J=10.4 Hz, 1H), 2.31 (s, 3H), 2.38 (t, J=10.4 Hz, 1H), 2.75 (d, J=11.2 Hz, 1H), 3.02 (d, J=10. Hz, 1H), 4.32 (s, 4H), 6.64 (dd, J=7.4, 1.0 Hz, 1H), 6.97 (dd, J=8.4, 7.4 Hz, 1H), 7.63-7.72 (m, 2H), 7.95 (s, 1H), 8.01 (s, 1H).

### Example 356—Synthesis of 1-(4-(2-Hydroxybutan-2-yl)Pyridin-2-yl)-N-(6-Methoxy-1-Methyl-1H-Indazol-7-yl)-1H-Pyrazole-4-Sulfonamide (I-315)

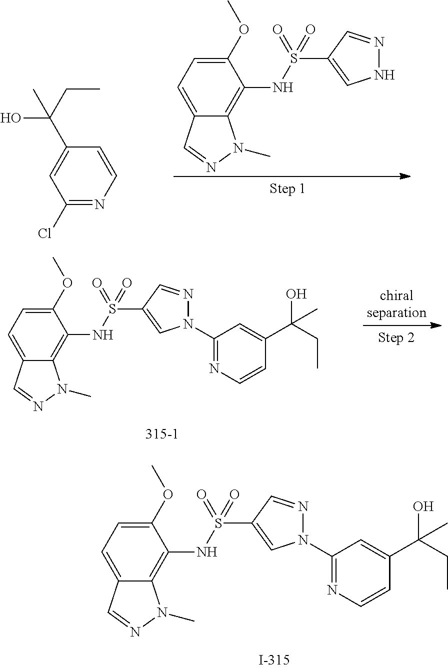

Step 1: Synthesis of 315-1: A mixture of 2-(2-chloropyridin-4-yl)butan-2-ol (150 mg, 0.808 mmol, 1 equiv), N-(6-methoxy-1-methylindazol-7-yl)-1H-pyrazole-4-sulfonamide (372.47 mg, 1.212 mmol, 1.5 equiv), EPhos (43.21 mg, 0.081 mmol, 0.1 equiv), EPhos Pd G4 (61.67 mg, 0.081 mmol, 0.1 equiv) and Cs₂CO₃ (789.76 mg, 2.424 mmol, 3 equiv) in DMF (1.5 mL) was stirred for 16 h at 110° C. under a nitrogen atmosphere. The resulting mixture was diluted with water (10 mL). The resulting mixture was extracted with DCM (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 1-[4-(2-hydroxybutan-2-yl)pyridin-2-yl]-N-(6-methoxy-1-methylindazol-7-yl)pyrazole-4-sulfonamide (315-1, 85 mg, 20.28%) as a white solid.

Step 2: Synthesis of 1-(4-(2-hydroxybutan-2-yl)pyridin-2-yl)-N-(6-methoxy-1-methyl-1H-indazol-7-yl)-1H-pyrazole-4-sulfonamide (I-315): 315-1 (85 mg) was purified by Prep-HPLC with the following conditions (column: Lux Sum Cellulose-2 2.12*25 cm, 5 μm; mobile phase A: hex(0.2% TFA), mobile phase B: IPA; flow rate: 20 mL/min; gradient: 35% B to 35% B in 34 min; wave length: 220/254 nm; RT1(min): 14.98; RT2(min): 17.91; sample solvent: EtOH:DCM=1:1; injection volume: 0.15 mL; number of runs: 20) to afford 1-{4-[2-hydroxybutan-2-yl]pyridin-2-yl}-N-(6-methoxy-1-methylindazol-7-yl)pyrazole-4-sulfonamide as a single steroeoisomer (1-315, 23.0 mg, 26.73%) as a white solid. LCMS (ES, m/z): [M+1]⁺=457; ¹H NMR: (400 MHz, methanol-d₄) δ 0.82 (t, J=7.6 Hz, 3H), 1.53 (s, 3H), 1.85 (q, J=7.6 Hz, 2H), 3.39 (s, 3H), 4.34 (s, 3H), 6.83 (d, J=8.8 Hz, 1H), 7.42 (dd, J=1.6, 5.2 Hz, 1H), 7.65 (d, J=8.8 Hz, 1H), 7.83 (s, 1H), 7.93 (s, 1H), 8.10 (d, J=1.6 Hz, 1H), 8.37 (d, J=5.2 Hz, 1H), 8.72 (s, 1H).

### Example 357—Synthesis of (S)—N-(6-Methoxy-1-Methyl-1H-Indazol-7-yl)-1-(4-(1-Methoxypropyl)Pyridin-2-yl)-1H-Pyrazole-4-Sulfonamide (I-317)

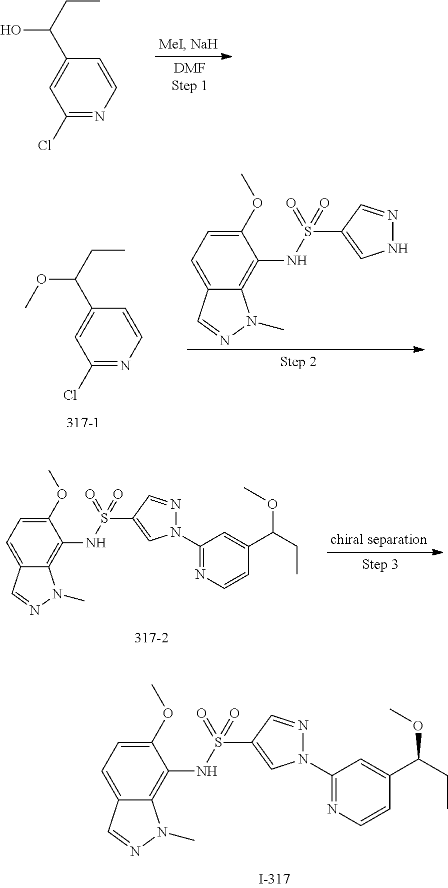 [SMILES: CCC(O)c1ccnc(Cl)c1 ; CCC(OC)c1ccnc(Cl)c1 ; COc1ccc2cnn(C)c2c1NS(=O)(=O)c1cn[nH]c1 ; CCC(OC)c1ccnc(-n2cc(S(=O)(=O)Nc3c(OC)ccc4cnn(C)c34)cn2)c1 ; CC[C@H](OC)c1ccnc(-n2cc(S(=O)(=O)Nc3c(OC)ccc4cnn(C)c34)cn2)c1]

Step 1: Synthesis of 317-1: A solution of 1-(2-chloropyridin-4-yl)propan-1-ol (600 mg, 3.496 mmol, 1 equiv) in DMF (5 mL) was treated with NaH (251.70 mg, 10.488 mmol, 3 equiv) for 30 min at 0° C. under a nitrogen atmosphere followed by the addition of Mel (0.44 mL, 6.992 mmol, 2 equiv) dropwise at room temperature. The resulting mixture was stirred for 2 h at room temperature under a nitrogen atmosphere. The reaction was quenched by the addition of water (10 mL) at 0° C. The resulting mixture was extracted with CH₂Cl₂ (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 20 min; detector: UV 254 nm. This resulted in 2-chloro-4-(1-methoxypropyl)pyridine (317-1, 600 mg, 73.95%) as a yellow oil.

Step 2: Synthesis of 317-2: To a stirred mixture of 2-chloro-4-(1-methoxypropyl)pyridine (200 mg, 1.077 mmol, 1 equiv) and N-(6-methoxy-1-methylindazol-7-yl)-1H-pyrazole-4-sulfonamide (331.09 mg, 1.077 mmol, 1 equiv) in DMSO (10 mL) was added Cs₂CO₃ (1053.01 mg, 3.231 mmol, 3 equiv) in portions at room temperature under a nitrogen atmosphere. The resulting mixture was stirred overnight at 140° C. under a nitrogen atmosphere. The resulting mixture was diluted with water (15 mL). The resulting mixture was extracted with EtOAc (3×15 mL). The combined organic layers were washed with brine (lx 50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in N-(6-methoxy-1-methylindazol-7-yl)-1-[4-(1-methoxypropyl)pyridin-2-yl]pyrazole-4-sulfonamide (317-2, 150 mg, 27.45%) as a light brown solid.

Step 3: Chiral Separation: N-(6-methoxy-1-methylindazol-7-yl)-1-[4-(1-methoxypropyl)pyridin-2-yl]pyrazole-4-sulfonamide (120 mg) was separated by Prep-chiral-HPLC with the following conditions (column: CHIRALPAKIH3; mobile phase A: hex(0.2% DEA): EtOH=80:20; flow rate: 1 mL/min; gradient: isocratic; injection volume: 3 mL) to afford N-(6-methoxy-1-methylindazol-7-yl)-1-{4-[(1S)-1-methoxypropyl] pyridin-2-yl}pyrazole-4-sulfonamide (I-317, 38.2 mg, 5.12%) as an off-white solid. LCMS (ES, m/z): [M+1]⁺=457; ¹H NMR (400 MHz, DMSO-d₆) δ 0.84 (t, J=7.4 Hz, 3H), 1.65-1.77 (m, 2H), 3.23 (s, 3H), 3.34 (s, 3H), 4.24 (s, 3H), 4.33 (t, J=6.4 Hz, 1H), 6.88 (d, J=8.8 Hz, 1H), 7.38 (dd, J=1.2, 5.2 Hz, 1H), 7.68 (d, J=8.8 Hz, 1H), 7.89 (s, 1H), 7.98 (s, 1H), 7.99 (d, J=4.4 Hz, 1H), 8.49 (d, J=5.2 Hz, 1H), 8.71 (s, 1H), 9.79 (s, 1H).

### Example 358—Synthesis of (R)—N-(6-Methoxy-1-Methyl-1H-Indazol-7-yl)-1-(4-(1-Methoxypropyl)Pyridin-2-yl)-1H-Pyrazole-4-Sulfonamide (I-318)

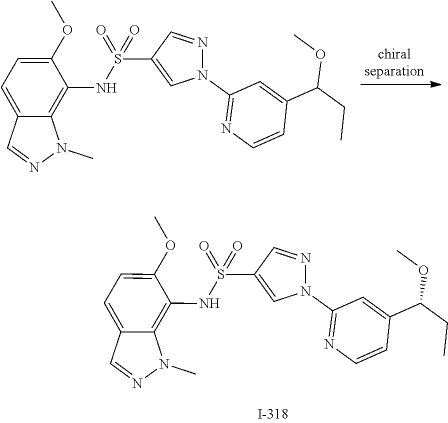 [SMILES: CCC(OC)c1ccnc(-n2cc(S(=O)(=O)Nc3c(OC)ccc4cnn(C)c34)cn2)c1 ; CC[C@@H](OC)c1ccnc(-n2cc(S(=O)(=O)Nc3c(OC)ccc4cnn(C)c34)cn2)c1]

N-(6-methoxy-1-methylindazol-7-yl)-1-[4-(1-methoxypropyl)pyridin-2-yl]pyrazole-4-sulfonamide (120 mg) was separated by Prep-chiral-HPLC with the following conditions (column: CHIRALPAKIH3; mobile phase A: hex(0.2% DEA): EtOH=80:20; flow rate: 1 mL/min; gradient: isocratic; injection volume: 3 L) to afford N-(6-methoxy-1-methylindazol-7-yl)-1-{4-[(1R)-1-methoxypropyl]pyridin-2-yl}pyrazole-4-sulfonamide (I-318, 54.9 mg, 7.35%) as an off-white solid. LCMS (ES, m/z): [M+1]=457; ¹H NMR (400 MHz, DMSO-d₆) δ 0.84 (t, J=7.4 Hz, 3H), 1.65-1.77 (m, 2H), 3.23 (s, 3H), 3.34 (s, 3H), 4.24 (s, 3H), 4.33 (t, J=6.4 Hz, 1H), 6.88 (d, J=8.8 Hz, 1H), 7.38 (dd, J=1.2, 5.2 Hz, 1H), 7.68 (d, J=8.8 Hz, 1H), 7.89 (s, 1H), 7.98 (s, 1H), 7.99 (s, 1H), 8.49 (d, J=5.2 Hz, 1H), 8.71 (s, 1H), 9.79 (s, 1H).

### Example 359—Synthesis of 1-(4-(Hydroxymethyl)Pyridin-2-yl)-N-(6-Methoxy-1-Methyl-1H-Indazol-7-yl)-1H-Pyrazole-4-Sulfonamide (I-323)

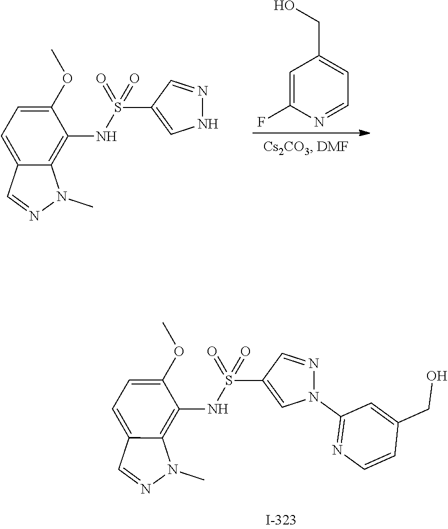 [SMILES: COc1ccc2cnn(C)c2c1NS(=O)(=O)c1cn[nH]c1 ; OCc1ccnc(F)c1 ; COc1ccc2cnn(C)c2c1NS(=O)(=O)c1cnn(-c2cc(CO)ccn2)c1]

A solution of N-(6-methoxy-1-methylindazol-7-yl)-1H-pyrazole-4-sulfonamide (150 mg, 0.488 mmol, 1 equiv), (2-fluoropyridin-4-yl)methanol (93.06 mg, 0.732 mmol, 1.5 equiv) and Cs₂CO₃ (477.07 mg, 1.464 mmol, 3 equiv) in DMF (1.5 mL) was stirred for 8 h at 120° C. under a nitrogen atmosphere. The resulting mixture was diluted with water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 1-[4-(hydroxymethyl)pyridin-2-yl]-N-(6-methoxy-1-methylindazol-7-yl)pyrazole-4-sulfonamide (I-323, 71.8 mg, 35.39%) as a white solid. LCMS (ES, m/z): [M+1]⁺=415; ¹H NMR: (400 MHz, methanol-d₄) δ 3.38 (s, 3H), 4.34 (s, 3H), 4.74 (s, 2H), 6.83 (d, J=8.8 Hz, 1H), 7.35 (d, J=5.2 Hz, 1H), 7.65 (d, J=8.8 Hz, 1H), 7.82 (s, 1H), 7.93 (s, 1H), 8.02 (s, 1H), 8.38 (d, J=5.2 Hz, 1H), 8.72 (s, 1H).

### Example 360—Synthesis of 1-(4-((1-Hydroxy-2-Methylpropan-2-yl)Amino)Pyridin-2-yl)-N-(1-Methyl-1H-Indazol-7-yl)-1H-Pyrazole-4-Sulfonamide (I-324)

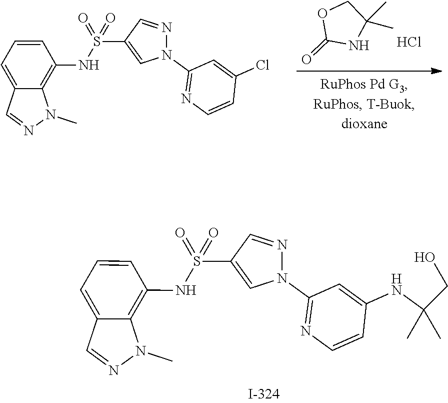

To a stirred mixture of 1-(4-chloropyridin-2-yl)-N-(1-methylindazol-7-yl) pyrazole-4-sulfonamide (100 mg, 0.257 mmol, 1 equiv) and 4,4-dimethyl-1,3-oxazolidin-2-one (59.22 mg, 0.514 mmol, 2 equiv) in dioxane (6 mL) were added RuPhos Pd G3 (43.02 mg, 0.051 mmol, 0.2 equiv), t-BuOK (57.72 mg, 0.514 mmol, 2 equiv) and RuPhos (24.00 mg, 0.051 mmol, 0.2 equiv) in portions at room temperature under a nitrogen atmosphere. The resulting mixture was stirred for 3 h at 100° C. under a nitrogen atmosphere. The resulting mixture was diluted with water (15 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 1-[4-(4,4-dimethyl-2-oxo-1,3-oxazolidin-3-yl) pyridin-2-yl]-N-(1-methylindazol-7-yl) pyrazole-4-sulfonamide (I-324, 29.3 mg, 25.29%) as a white solid. LCMS (ES, m/z): [M+H]⁺=442; ¹H NMR: (400 MHz, DMSO-d₆) δ 1.29 (s, 6H), 3.45 (d, J=5.8 Hz, 2H), 4.28 (s, 3H), 4.95 (t, J=4.0 Hz, 1H), 6.60 (s, 1H), 6.69 (t, J=8.0 Hz, 2H), 6.97 (t, J=8.0 Hz, 1H), 7.25 (d, J=2.2 Hz, 1H), 7.68 (s, 1H), 7.93-7.86 (m, 2H), 8.07 (s, 1H), 8.66 (s, 1H), 9.95 (s, 1H).

### Example 361—Synthesis of N-(1-Methyl-6-(Trifluoromethoxy)-1H-Indazol-7-yl)-1-(4-(Trifluoromethyl)Pyridin-2-yl)-1H-Pyrazole-4-Sulfonamide (I-327)

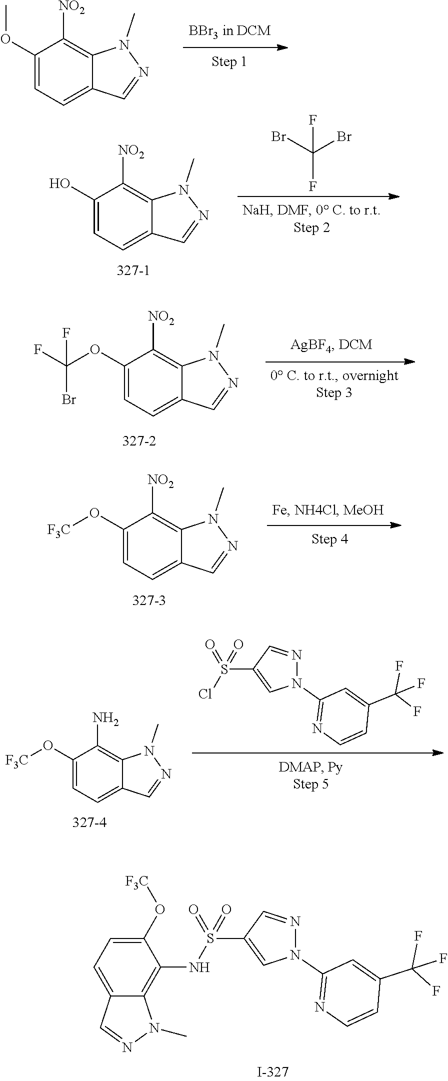

Step 1: Synthesis of 327-1: To a stirred mixture of 6-methoxy-1-methyl-7-nitroindazole (2.3 g, 11.101 mmol, 1 equiv) in DCM (20 mL) was added BBr₃ (4.17 g, 16.652 mmol, 1.5 equiv) in portions at −78° C. under a nitrogen atmosphere. The resulting mixture was stirred for 3 h at room temperature under a nitrogen atmosphere. The reaction was quenched with sat. sodium hyposulfite (aq.) at 0° C. The resulting mixture was extracted with EtOAc (2×50 mL). The combined organic layers were washed with brine (1×20 mL) and dried over anhydrous MgSO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 1-methyl-7-nitroindazol-6-ol (327-1, 1.8 g, 83.94%) as an off-white solid.

Step 2: Synthesis of 327-2: To a solution of 1-methyl-7-nitroindazol-6-ol (1 g, 5.177 mmol, 1 equiv) in DMF (10 mL, 129.215 mmol, 24.96 equiv) was added sodium hydride (60% in oil) at 0° C. The mixture was stirred for 15 min. Dibromodifluoromethane (5.43 g, 25.885 mmol, 5 equiv) was added and the mixture was allowed to warm to rt and stirred for 4 h. The reaction mixture was quenched with water and extracted with DCM (3×25 mL). The combined organic layers were washed with brine (1×20 mL) and dried over anhydrous MgSO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 6-(bromodifluoromethoxy)-1-methyl-7-nitroindazole (327-2, 500 mg, 29.99%) as a yellow oil.

Step 3: Synthesis of 327-3: Into a 50 mL round-bottom flask were added 6-(bromodifluoromethoxy)-1-methyl-7-nitroindazole (500 mg, 1.552 mmol, 1 equiv) and AgBF₄ (604.44 mg, 3.104 mmol, 2 equiv) in DCM (10 mL) at 0° C. The resulting mixture was stirred overnight at room temperature under a nitrogen atmosphere. The reaction was quenched with ice at 0° C. The resulting mixture was extracted with EtOAc (2×50 mL). The combined organic layers were washed with brine (2×10 mL) and dried over anhydrous MgSO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-methyl-7-nitro-6-(trifluoromethoxy)indazole (327-3, 200 mg, 49.33%) as a yellow oil.

Step 4: Synthesis of 327-4: A solution 1-methyl-7-nitro-6-(trifluoromethoxy)indazole (200 mg, 0.766 mmol, 1 equiv), Fe (213.83 mg, 3.830 mmol, 5 equiv), NH₄Cl (204.82 mg, 3.830 mmol, 5 equiv) and CH₃COOH (91.98 mg, 1.532 mmol, 2 equiv) in MeOH (5 mL, 123.494 mmol, 161.26 equiv) was stirred overnight at 80° C. under a nitrogen atmosphere. The resulting mixture was filtered and the filter cake was washed with EtOAc (2×20 mL). The filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 1-methyl-6-(trifluoromethoxy)indazol-7-amine (327-4, 100 mg, 56.48%) as an off-white solid.

Step 5: Synthesis of I-327): A solution of 1-methyl-6-(trifluoromethoxy)indazol-7-amine (50 mg, 0.216 mmol, 1 equiv), 1-[4-(trifluoromethyl)pyridin-2-yl]pyrazole-4-sulfonyl chloride (80.89 mg, 0.259 mmol, 1.2 equiv) and DMAP (2.64 mg, 0.022 mmol, 0.1 equiv) in pyridine was stirred for 8 h at 80° C. under a nitrogen atmosphere. The reaction was quenched with water at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-[1-methyl-6-(trifluoromethoxy)indazol-7-yl]-1-[4-(trifluoromethyl)pyridin-2-yl]pyrazole-4-sulfonamide (I-327, 8.2 mg, 7.49%) as an off-white solid. LCMS (ES, m/z): [M+H]=507; ¹H NMR: (400 MHz, DMSO-d₆): δ 4.30 (s, 3H), 7.07 (d, J=9.2 Hz, 1H), 7.83 (s, 1H), 7.89 (d, J=5.2 Hz, 1H), 8.14 (s, 1H), 8.19 (d, J=6.6 Hz, 2H), 8.83 (d, J=5.2 Hz, 1H), 8.90 (s, 1H), 10.47 (s, 1H).

### Example 362—Synthesis of N-(6-Methoxy-1-(Methyl-D3)-1H-Indazol-7-yl)-N-Methyl-1-(4-(Trifluoromethyl)Pyridin-2-yl)-1H-Pyrazole-4-Sulfonamide (I-336)

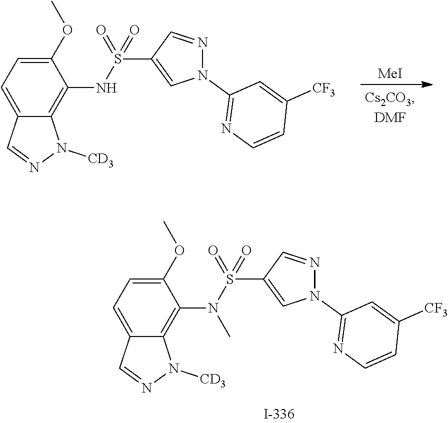 [SMILES: "MeI" ; [2H]C([2H])([2H])n1ncc2ccc(OC)c(NS(=O)(=O)c3cnn(-c4cc(C(F)(F)F)ccn4)c3)c21 ; [2H]C([2H])([2H])n1ncc2ccc(OC)c(N(C)S(=O)(=O)c3cnn(-c4cc(C(F)(F)F)ccn4)c3)c21]

A solution of N-(6-methoxy-1-(methyl-d3)-1H-indazol-7-yl)-1-(4-(trifluoromethyl)pyridin-2-yl)-1H-pyrazole-4-sulfonamide (100 mg, 0.220 mmol, 1 equiv), Cs₂CO₃ (214.62 mg, 0.660 mmol, 3 equiv) and Mel (0.07 mL, 1.100 mmol, 5 equiv) in DMF (3 mL) was stirred for 2 h at room temperature under a nitrogen atmosphere. The resulting mixture was diluted with water (10 mL). The resulting mixture was extracted with CH₂Cl₂ (3×10 mL). The combined organic layers were washed with brine (1×30 mL) and dried over anhydrous Na₂SO₄. After filtration, the filtrate was concentrated under reduced pressure. The crude product (80 mg) was purified by Prep-HPLC with the following conditions (column: Xselect CSH C18 OBD Column 30*150 mm 6 m; mobile phase A: water(0.1% FA), mobile phase B: ACN; flow rate: 60 mL/min; gradient: 50% B to 62% B in 7 min; wave length: 254 nm/220 nm; RT1(min): 6.3) to afford N-(6-methoxy-1-(methyl-d3)-1H-indazol-7-yl)-N-methyl-1-(4-(trifluoromethyl)pyridin-2-yl)-1H-pyrazole-4-sulfonamide (I-336, 26.3 mg, 25.46%) as an off-white solid. LCMS (ES, m/z): [M+1]⁺=470; ¹H NMR: ¹H NMR (400 MHz, acetonitrile-d₃) δ 3.35 (s, 3H), 3.48 (s, 3H), 6.90 (d, J=8.8 Hz, 1H), 7.71 (d, J=5.2 Hz, 1H), 7.73 (d, J=8.8 Hz, 1H), 7.93 (s, 1H), 8.02 (d, J=0.8 Hz, 1H), 8.27 (s, 1H), 8.73 (d, J=5.2 Hz, 1H), 8.91 (d, J=0.8 Hz, 1H).

### Example 363—Synthesis of N-(1-Methyl-6-(Trifluoromethoxy)-1H-Indazol-7-yl)-1-(4-(Trifluoromethyl)Pyridin-2-yl)-N-((1-(4-(Trifluoromethyl)Pyridin-2-yl)-1H-Pyrazol-4-yl)Sulfonyl)-1H-Pyrazole-4-Sulfonamide (I-337)

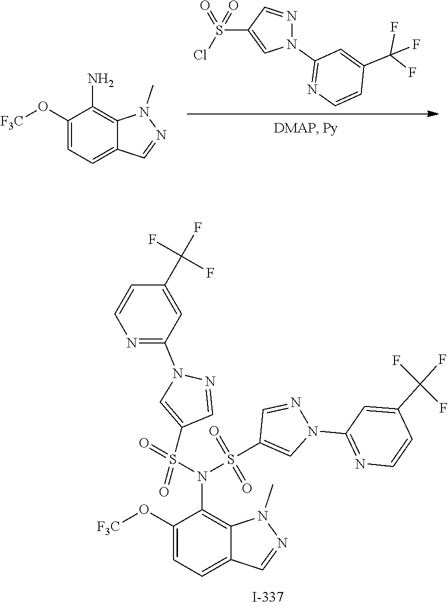 [SMILES: Cn1ncc2ccc(OC(F)(F)F)c(N)c21 ; O=S(=O)(Cl)c1cnn(-c2cc(C(F)(F)F)ccn2)c1 ; Cn1ncc2ccc(OC(F)(F)F)c(N(S(=O)(=O)c3cnn(-c4cc(C(F)(F)F)ccn4)c3)S(=O)(=O)c3cnn(-c4cc(C(F)(F)F)ccn4)c3)c21]

A solution of 1-methyl-6-(trifluoromethoxy)indazol-7-amine (50 mg, 0.216 mmol, 1 equiv) 1-[4-(trifluoromethyl)pyridin-2-yl]pyrazole-4-sulfonyl chloride (80.89 mg, 0.259 mmol, 1.2 equiv) and DMAP (2.64 mg, 0.022 mmol, 0.1 equiv) in pyridine (5 mL) was stirred for 8 h at 80° C. under a nitrogen atmosphere. The reaction was quenched with water at room temperature. The resulting mixture was extracted with EtOAc (2×20 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 70% to 80% gradient in 10 min; detector: UV 254 nm. This resulted in N-[1-methyl-6-(trifluoromethoxy)indazol-7-yl]-1-[4-(trifluoromethyl)pyridin-2-yl]-N-{1-[4-(trifluoromethyl)pyridin-2-yl]pyrazol-4-ylsulfonyl}pyrazole-4-sulfonamide (I-337, 14.6 mg, 8.64%) as an off-white solid. LCMS (ES, m/z): [M+H]⁺=782; ¹H NMR: (400 MHz, DMSO-d₆): δ 3.83 (s, 3H), 7.23 (d, J=8.6 Hz, 1H), 7.96 (m, J=5.0 Hz, 2H), 8.13 (d, J=9.0 Hz, 1H), 8.25 (m, 2H), 8.31 (s, 1H), 8.45 (s, 2H), 8.85 (d, J=5.2 Hz, 2H), 9.32 (s, 2H).

### Example 364—Synthesis of N-(1-(Methyl-D3)-1H-Indazol-7-yl)-1-(4-(Trifluoromethyl)Pyridin-2-yl-5-D)-1H-Pyrazole-4-Sulfonamide (I-338)

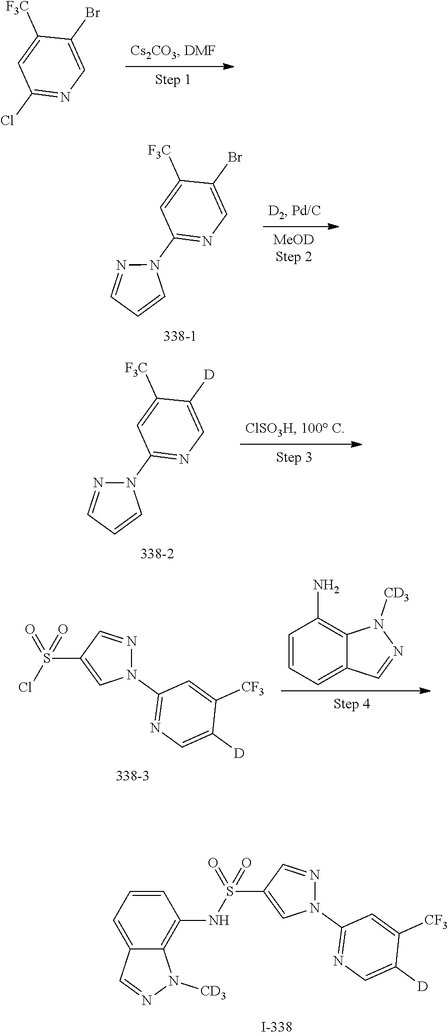 [SMILES: FC(F)(F)c1cc(Cl)ncc1Br ; FC(F)(F)c1cc(-n2cccn2)ncc1Br ; [2H]c1cnc(-n2cccn2)cc1C(F)(F)F ; [2H]c1cnc(-n2cc(S(=O)(=O)Cl)cn2)cc1C(F)(F)F ; [2H]C([2H])([2H])n1ncc2cccc(N)c21 ; [2H]c1cnc(-n2cc(S(=O)(=O)Nc3cccc4cnn(C([2H])([2H])[2H])c34)cn2)cc1C(F)(F)F]

Step 1: Synthesis of 338-1: A mixture of 5-bromo-2-chloro-4-(trifluoromethyl)pyridine (1.5 g, 5.759 mmol, 1 equiv), Cs₂CO₃ (5.63 g, 17.277 mmol, 3 equiv) and pyrazole (0.39 g, 5.759 mmol, 1 equiv) in DMF (15 mL) was stirred for 2 h at 80° C. The reaction was quenched with water (50 ml) at 25° C. The precipitated solids were collected by filtration and washed with water (3×20 mL) to afford 5-bromo-2-(1H-pyrazol-1-yl)-4-(trifluoromethyl)pyridine (338-1, 1.5 g, 86.59%) as an off-white solid.

Step 2: Synthesis of 338-2: To a solution of 5-bromo-2-(1H-pyrazol-1-yl)-4-(trifluoromethyl)pyridine (100.00 mg, 0.342 mmol, 1 equiv) in 5 mL CD₃OD was added Pd/C (10%, 3.64 mg) under a nitrogen atmosphere in a 50 mL round-bottom flask. The mixture was deuterated at room temperature for 1 h under a deuterium atmosphere using a deuterium balloon, filtered through a celite pad and concentrated under reduced pressure. The residue was purified by silica gel column chromatography, eluted with PE/EA (40:1) to afford 2-(1H-pyrazol-1-yl)-4-(trifluoromethyl)pyridine-5-d (338-2, 50.00 mg, 66.21%) as an off-white oil.

Step 3: Synthesis of 338-3: A mixture of 2-(1H-pyrazol-1-yl)-4-(trifluoromethyl)pyridine-5-d (260.00 mg, 1.214 mmol, 1 equiv) in HSO₃Cl (4 mL, 0.094 mmol) was stirred for 16 h at 100° C. The reaction was quenched by the addition of water/ice (20 mL) at room temperature. The resulting mixture was extracted with EtOAc (3×15 mL). The combined organic layers were washed with brine (1×45 mL) and dried over anhydrous Na₂SO₄. After filtration, the filtrate was concentrated under reduced pressure to afford 1-(4-(trifluoromethyl) pyridin-2-yl-5-d)-1H-pyrazole-4-sulfonyl chloride (338-3, 180.00 mg, 44.15%) as an off-white solid.

Step 4: Synthesis of N-(1-(methyl-d3)-1H-indazol-7-yl)-1-(4-(trifluoromethyl) pyridin-2-yl-5-d)-1H-pyrazole-4-sulfonamide (I-338): A solution of 1-(4-(trifluoromethyl) pyridin-2-yl-5-d)-1H-pyrazole-4-sulfonyl chloride (400.00 mg, 1.279 mmol, 1 equiv) and 1-(methyl-d₃)-1H-indazol-7-amine (192.15 mg, 1.279 mmol, 1 equiv) in pyridine (5 mL) was stirred for 1 h at 80° C. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 60% gradient in 10 min; detector: UV 254 nm, to afford N-(1-(methyl-d₃)-1H-indazol-7-yl)-1-(4-(trifluoromethyl)pyridin-2-yl-5-d)-1H-pyrazole-4-sulfonamide (I-338, 152.2 mg, 27.23%) as an off-white solid. LCMS (ES, m/z): [M+H]⁺=427; ¹H NMR: (400 MHz, DMSO-d₆) δ 6.72 (d, J=8.0 Hz, 1H), 6.97 (t, J=8.0 Hz, 1H), 7.71 (d, J=7.6 Hz, 1H), 8.10 (s, 1H), 8.11 (s, 1H), 8.21 (s, 1H), 8.83 (s, 1H), 8.88 (s, 1H), 10.10 (s, 1H).

### Example 365—Synthesis of 1-(4-(1-Hydroxyethyl)Pyridin-2-yl)-N-(6-Methoxy-1-Methyl-1H-Indazol-7-yl)-1H-Pyrazole-4-Sulfonamide (I-339)

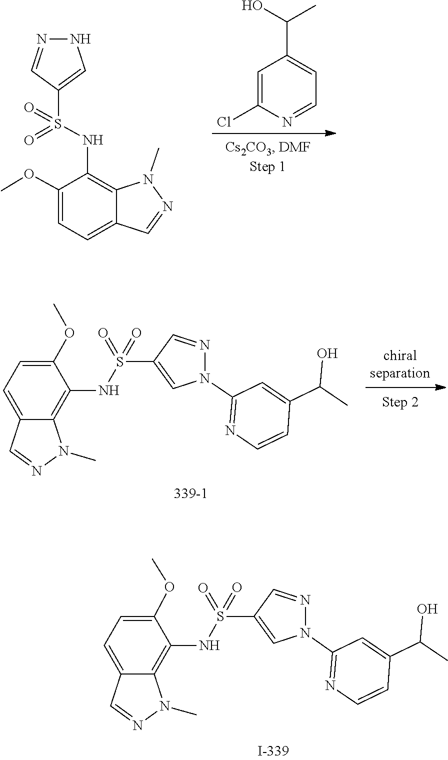

Step 1: Synthesis of 339-1: To a stirred solution of N-(6-methoxy-1-methylindazol-7-yl)-1H-pyrazole-4-sulfonamide (200 mg, 0.651 mmol, 1 equiv) and 1-(2-chloropyridin-4-yl)ethanol (102.56 mg, 0.651 mmol, 1 equiv) in DMF (2 mL) was added EPhos (34.80 mg, 0.065 mmol, 0.1 equiv) and EPhos Pd G4 (59.78 mg, 0.065 mmol, 0.1 equiv) in portions at 140° C. under a nitrogen atmosphere. The resulting mixture was stirred overnight at 140° C. under a nitrogen atmosphere. The resulting mixture was diluted with water (10 mL) and extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: MeCN in water (0.1% FA), 10% to 100% gradient in 20 min; detector: UV 254 nm. The resulting mixture was concentrated under vacuum. This resulted in 1-[4-(1-hydroxyethyl)pyridin-2-yl]-N-(6-methoxy-1-methylindazol-7-yl)pyrazole-4-sulfonamide (339-1, 104 mg, 33.57%) as a white solid.

Step 2: Synthesis of 1-(4-(1-hydroxyethyl)pyridin-2-yl)-N-(6-methoxy-1-methyl-1H-indazol-7-yl)-1H-pyrazole-4-sulfonamide (I-339): The crude product 339-1 (104 mg crude) was purified by chiral HPLC with the following conditions (column: Lux3umCellulose2; mobile phase A: hex(0.2% TFA): IPA=65:35; flow rate: 1 mL/min; gradient: isocratic; injection volume: 3 L) to afford 1-(4-(1-hydroxyethyl)pyridin-2-yl)-N-(6-methoxy-1-methyl-1H-indazol-7-yl)-1H-pyrazole-4-sulfonamide as a single steroeoisomer (I-339, 22.2 mg, 21.24%) as a white solid. LCMS (ES, m/z): [M+H]⁺ 429; ¹H NMR: (400 MHz, acetonitrile-d₃) δ 1.46 (d, J=6.4 Hz, 3H), 3.34 (s, 3H), 3.60 (s, 1H), 4.31 (s, 3H), 4.94 (t, J=4.0 Hz, 1H), 6.81 (d, J=8.8 Hz, 1H), 7.38 (d, J=5.0 Hz, 1H), 7.68 (d, J=8.8 Hz, 1H), 7.84 (s, 1H), 7.93 (s, 1H), 7.99 (s, 1H), 8.39 (d, J=5.0 Hz, 1H), 8.69 (s, 1H).

### Example 366—Synthesis of (R)-1-(4-(1-Hydroxyethyl)Pyridin-2-yl)-N-(6-Methoxy-1-Methyl-1H-Indazol-7-yl)-1H-Pyrazole-4-Sulfonamide (I-340)

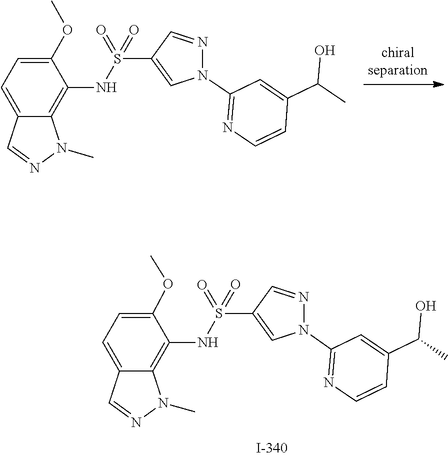 [SMILES: COc1ccc2cnn(C)c2c1NS(=O)(=O)c1cnn(-c2cc(C(C)O)ccn2)c1 ; COc1ccc2cnn(C)c2c1NS(=O)(=O)c1cnn(-c2cc([C@@H](C)O)ccn2)c1]

The crude product 1-(4-(1-hydroxyethyl)pyridin-2-yl)-N-(6-methoxy-1-methyl-1H-indazol-7-yl)-1H-pyrazole-4-sulfonamide (339-1, 104 mg crude) was purified by chiral HPLC with the following conditions (column: Lux3umCellulose2; mobile phase A: hex(0.2% TFA): IPA=65:35; flow rate: 1 mL/min; gradient: isocratic; injection volume: 3 L) to afford (R)-1-(4-(1-hydroxyethyl)pyridin-2-yl)-N-(6-methoxy-1-methyl-1H-indazol-7-yl)-1H-pyrazole-4-sulfonamide (I-340, 20.5 mg, 19.55%) as a white solid. LCMS (ES, m/z): [M+H]⁺ 429; ¹H NMR: (400 MHz, acetonitrile-d₃) δ 1.46 (d, J=6.4 Hz, 3H), 3.39 (s, 3H), 3.60 (s, 1H), 4.31 (s, 3H), 4.94 (t, J=4.0 Hz, 1H), 6.81 (d, J=8.8 Hz, 1H), 7.38 (d, J=5.0 Hz, 1H), 7.68 (d, J=8.8 Hz, 1H), 7.84 (s, 1H), 7.93 (s, 1H), 7.99 (s, 1H), 8.39 (d, J=5.2 Hz, 1H), 8.69 (s, 1H).

### Example 367—1-(4-(1-Fluoroallyl)Pyridin-2-yl)-N-(6-Methoxy-1-Methyl-1H-Indazol-7-yl)-1H-Pyrazole-4-Sulfonamide (I-343)

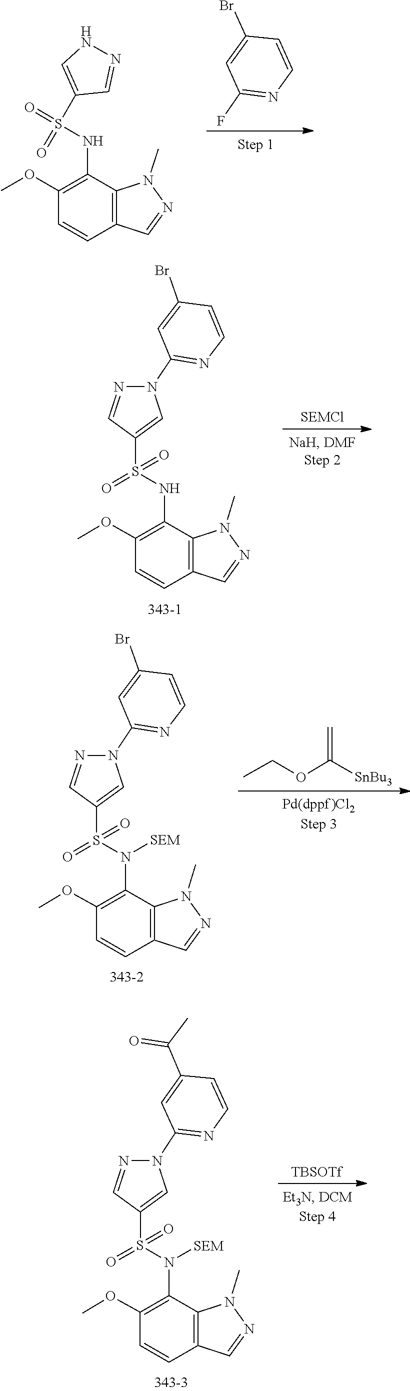

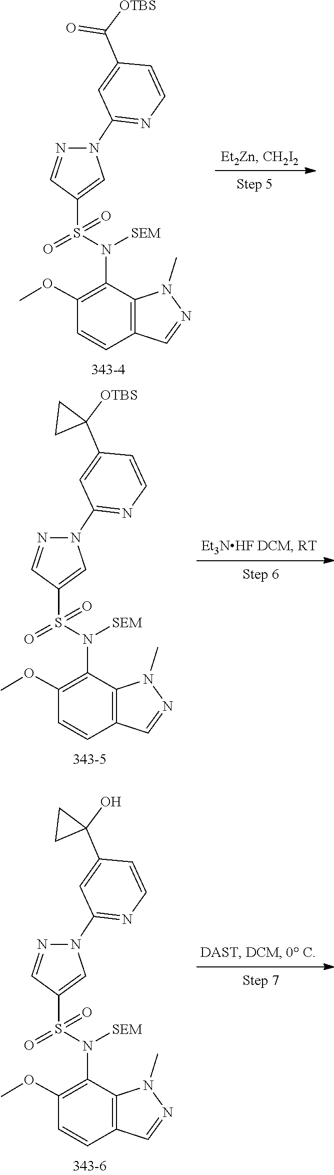

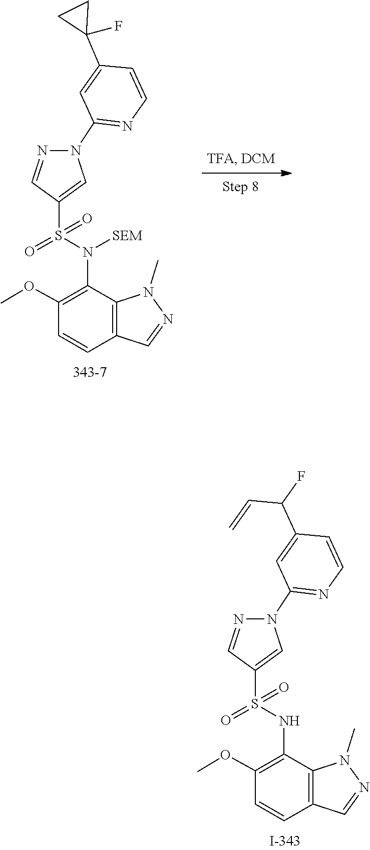

Step 1: Synthesis of 343-1: To a stirred mixture of N-(6-methoxy-1-methylindazol-7-yl)-1H-pyrazole-4-sulfonamide (3 g, 9.761 mmol, 1 equiv) and 4-bromo-2-fluoropyridine (1717.91 mg, 9.761 mmol, 1 equiv) in DMF (50 mL) was added Cs₂CO₃ (9541.44 mg, 29.283 mmol, 3 equiv) in portions at room temperature under a nitrogen atmosphere. The resulting mixture was stirred overnight at 80° C. under a nitrogen atmosphere. The resulting mixture was diluted with water (100 mL). The resulting mixture was extracted with EtOAc (3×100 mL). The combined organic layers were washed with brine (200 mL) and dried over anhydrous Na₂SO₄. After filtration, the filtrate was concentrated under reduced pressure. The resulting mixture was concentrated under vacuum. The residue was purified by reversed-phase flash chromatography with the following conditions: column: C18 silica gel; mobile phase, MeOH in water, 0% to 100% gradient in 30 min; detector: UV 254 nm. The resulting mixture was concentrated under vacuum. This resulted in 1-(4-bromopyridin-2-yl)-N-(6-methoxy-1-methylindazol-7-yl)pyrazole-4-sulfonamide (343-1, 2 g, 39.80%) as an off-white solid.

Step 2: Synthesis of 343-2: To a stirred mixture of 1-(4-bromopyridin-2-yl)-N-(6-methoxy-1-methylindazol-7-yl)pyrazole-4-sulfonamide (2 g, 4.317 mmol, 1 equiv) and [2-(chloromethoxy)ethyl]trimethylsilane (2.16 g, 12.951 mmol, 3 equiv) in DMF (20 mL) was added NaH (310.78 mg, 12.951 mmol, 3 equiv) in portions at room temperature under a nitrogen atmosphere. The resulting mixture was stirred for 4 h at room temperature under a nitrogen atmosphere. The reaction was quenched by the addition of water/ice (20 mL) at room temperature. The resulting mixture was extracted with EtOAc (3×15 mL). The combined organic layers were washed with brine (1×45 mL) and dried over anhydrous Na₂SO₄. After filtration, the filtrate was concentrated under reduced pressure. This resulted in 1-(4-bromopyridin-2-yl)-N-(6-methoxy-1-methylindazol-7-yl)-N-{[2-(trimethylsilyl)ethoxy]methyl}pyrazole-4-sulfonamide (343-2, 2.3 g, 71.81%) as an off-white solid.

Step 3: Synthesis of 343-3: To a stirred mixture of 1-(4-bromopyridin-2-yl)-N-(6-methoxy-1-methylindazol-7-yl)-N-{[2-(trimethylsilyl)ethoxy]methyl}pyrazole-4-sulfonamide (2.3 g, 3.875 mmol, 1 equiv) and tributyl(1-ethoxyethenyl)stannane (2.80 g, 7.750 mmol, 2 equiv) in 1,4-dioxane (25 mL) was added Pd(dppf)Cl₂ (567.05 mg, 0.775 mmol, 0.2 equiv) in portions at room temperature under a nitrogen atmosphere. The resulting mixture was stirred overnight at 95° C. under a nitrogen atmosphere. The reaction was monitored by LCMS. After cooling it to room temperature, a 2 M hydrochloric acid solution was added. The resulting mixture was stirred for 2 h at room temperature under a nitrogen atmosphere. The aqueous layer was extracted with EtOAc (3×30 mL). The resulting mixture was concentrated under vacuum. The residue was purified by reversed-phase flash chromatography with the following conditions: column: C18 silica gel; mobile phase: MeCN in water, 0% to 100% gradient in 30 min; detector: UV 254 nm. This resulted in 1-(4-acetylpyridin-2-yl)-N-(6-methoxy-1-methylindazol-7-yl)-N-{[2-(trimethylsilyl)ethoxy]methyl}pyrazole-4-sulfonamide (343-3, 2 g, 88.08%) as a light brown oil.

Step 4: Synthesis of 343-4: To a stirred mixture of N-(6-methoxy-1-methylindazol-7-yl)-1-[4-(prop-1-en-2-yl)pyridin-2-yl]-N-{[2-(trimethylsilyl)ethoxy]methyl}pyrazole-4-sulfonamide (2 g, 3.605 mmol, 1 equiv) and tert-butyldimethylsilyl trifluoromethanesulfonate (1.43 g, 5.407 mmol, 1.5 equiv) in DCM (25 mL) was added TEA (1.09 g, 10.815 mmol, 3 equiv) dropwise at 0° C. under a nitrogen atmosphere. The resulting mixture was stirred overnight at room temperature under a nitrogen atmosphere. The resulting mixture was diluted with water (30 mL). The resulting mixture was extracted with EtOAc (3×30 mL). The combined organic layers were washed with brine (50 mL) and dried over anhydrous Na₂SO₄. After filtration, the filtrate was concentrated under reduced pressure. The resulting mixture was concentrated under vacuum. The residue was purified by reversed-phase flash chromatography with the following conditions: column: C18 silica gel; mobile phase: MeCN in water, 0% to 100% gradient in 30 min; detector: UV 254 nm. This resulted in 1-(4-{1-[(tert-butyldimethylsilyl)oxy]ethenyl}pyridin-2-yl)-N-(6-methoxy-1-methylindazol-7-yl)-N-{[2-(trimethylsilyl)ethoxy]methyl}pyrazole-4-sulfonamide (343-4, 2 g, 78.54%) as a light yellow oil.

Step 5: Synthesis of 343-5: To a stirred mixture of 1-(4-{1-[(tert-butyldimethylsilyl)oxy]ethenyl}pyridin-2-yl)-N-(6-methoxy-1-methylindazol-7-yl)-N-{[2-(trimethylsilyl)ethoxy]methyl}pyrazole-4-sulfonamide (1.37 g, 2.042 mmol, 1 equiv) and ZnEt₂ (42.03 mL, 408.400 mmol, 200 equiv) in DCM (15 mL) was added CH₂I₂ (3.42 mL, 40.840 mmol, 20 equiv) in portions at 0° C. under a nitrogen atmosphere. The resulting mixture was stirred for 24 h at room temperature under a nitrogen atmosphere. The reaction was quenched with water (20 mL) at 0° C. The aqueous layer was extracted with EtOAc (3×20 mL). The combined organic layers were washed with brine (60 mL) and dried over anhydrous Na₂SO₄. After filtration, the filtrate was concentrated under reduced pressure. The residue was purified by silica gel column chromatography and eluted with PE/EA (2:1) to afford 1-(4-{1-[(tert-butyldimethylsilyl)oxy] cyclopropyl}pyridin-2-yl)-N-(6-methoxy-1-methylindazol-7-yl)-N-{[2-(trimethylsilyl)ethoxy]methyl}pyrazole-4-sulfonamide (343-5, 150 mg, 9.65%) as a light yellow oil

Step 6: Synthesis of 343-6: A mixture of 1-(4-{1-[(tert-butyldimethylsilyl)oxy]cyclopropyl}pyridin-2-yl)-N-(6-methoxy-1-methylindazol-7-yl)-N-{[2-(trimethylsilyl)ethoxy]methyl}pyrazole-4-sulfonamide (150 mg, 0.219 mmol, 1.00 equiv) and Et₃N·3HF (105.91 mg, 0.657 mmol, 3 equiv) in DCM (3 mL) was stirred for 2 h at room temperature under a nitrogen atmosphere. The reaction was monitored by LCMS. The resulting mixture was concentrated under reduced pressure. The residue was purified by silica gel column chromatography and eluted with PE/EA (5:1) to afford 1-[4-(1-hydroxycyclopropyl)pyridin-2-yl]-N-(6-methoxy-1-methylindazol-7-yl)-N-{[2-(trimethylsilyl)ethoxy]methyl}pyrazole-4-sulfonamide (343-6, 90 mg, 64.81%) as a light yellow oil.

Step 7: Synthesis of 343-7: A mixture of 1-[4-(1-hydroxycyclopropyl)pyridin-2-yl]-N-(6-methoxy-1-methylindazol-7-yl)-N-{[2-(trimethylsilyl)ethoxy]methyl}pyrazole-4-sulfonamide (85 mg, 0.149 mmol, 1 equiv) and DAST (72.02 mg, 0.447 mmol, 3 equiv) in DCM (2 mL) was stirred overnight at room temperature under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by silica gel column chromatography and eluted with PE/EA (5:1) to afford 1-[4-(1-fluorocyclopropyl)pyridin-2-yl]-N-(6-methoxy-1-methylindazol-7-yl)-N-{[2-(trimethylsilyl)ethoxy]methyl}pyrazole-4-sulfonamide (343-7, 80 mg, 84.41%) as a light yellow oil.

Step 8: 1-(4-(1-fluoroallyl)pyridin-2-yl)-N-(6-methoxy-1-methyl-1H-indazol-7-yl)-1H-pyrazole-4-sulfonamide (I-343): A mixture of 1-[4-(1-fluorocyclopropyl)pyridin-2-yl]-N-(6-methoxy-1-methylindazol-7-yl)-N-{[2-(trimethylsilyl)ethoxy]methyl}pyrazole-4-sulfonamide (90 mg, 0.157 mmol, 1 equiv) and TFA (53.75 mg, 0.471 mmol, 3 equiv) in DCM (2 mL) was stirred for 2 h at room temperature under a nitrogen atmosphere. The reaction was monitored by LCMS. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product (30 mg) was purified by Prep-HPLC with the following conditions (column: XBridge Shield RP18 OBD column, 30*150 mm, 7 m; mobile phase A: water (10 mmol/L NH₄HCO₃+0.05% NH₃H₂O), mobile phase B: ACN; flow rate: 60 mL/min; gradient: 5% B to 30% B in 7 min; wave length: 254 nm/222 nm nm; RT1(min): 6.22; number of runs: 2) to afford 1-[4-(1-fluoroprop-2-en-1-yl)pyridin-2-yl]-N-(6-methoxy-1-methylindazol-7-yl)pyrazole-4-sulfonamide (I-343, 1.2 mg, 1.65%) as an off-white solid. LCMS (ES, m/z): [M+1]⁺=443; ¹H NMR (400 MHz, DMSO-d₆) δ 3.34 (s, 3H), 4.25 (s, 3H), 5.38 (s, 1H), 5.50 (s, 1H), 5.81 (s, 1H), 6.15 (s, 1H), 6.87 (d, J=8.8 Hz, 1H), 7.64 (d, J=6.0 Hz, 1H), 7.67 (d, J=8.0 Hz, 1H), 7.99 (d, J=10.8 Hz, 1H), 8.00 (s, 1H), 8.05 (s, 1H), 8.54 (d, J=5.2 Hz, 1H), 8.72 (s, 1H).

### Example 368—Synthesis of N-(6-Methoxy-1-(Methyl-D3)-1H-Pyrazolo[4,3-C]Pyridin-7-yl)-1-(4-(Trifluoromethyl)Pyridin-2-yl)-1H-Pyrazole-4-Sulfonamide (I-352)

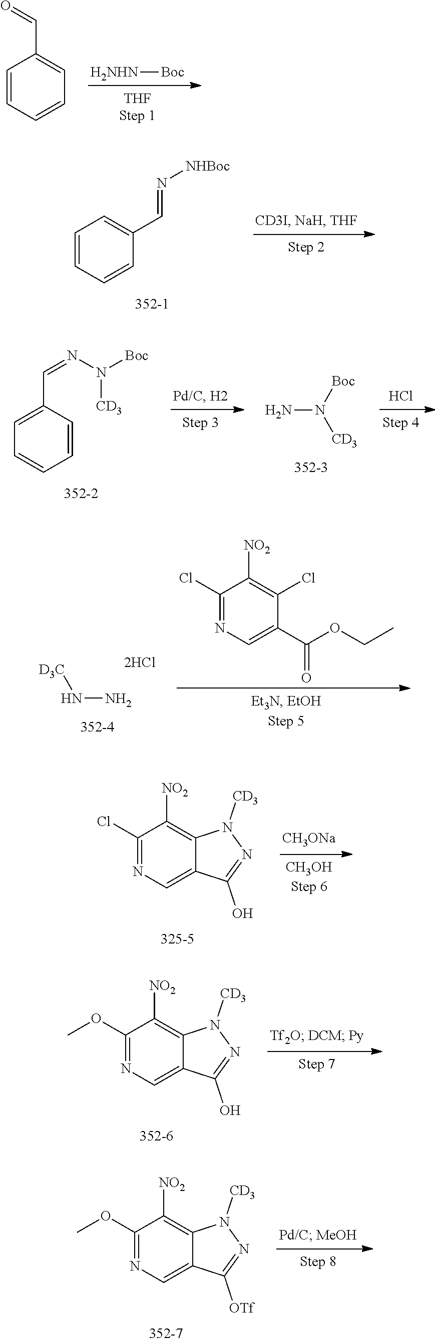

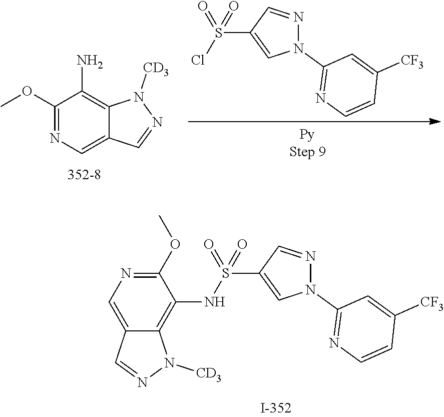 [SMILES: [2H]C([2H])([2H])n1ncc2cnc(OC)c(N)c21 ; O=S(=O)(Cl)c1cnn(-c2cc(C(F)(F)F)ccn2)c1 ; [2H]C([2H])([2H])n1ncc2cnc(OC)c(NS(=O)(=O)c3cnn(-c4cc(C(F)(F)F)ccn4)c3)c21]

Step 1: Synthesis of 352-1: A solution of tert-butoxycarbohydrazide (20 g, 151.328 mmol, 1.00 equiv) in tetrahydrofuran (250 mL) was treated with benzaldehyde (20.00 mL, 196.726 mmol, 1.3 equiv) overnight at room temperature under a nitrogen atmosphere. The product was precipitated by the addition of PE. The precipitated solids were collected by filtration and washed with PE (2×20 mL). The resulting solid was dried under vacuum to give 352-1 (27.4 g, 82.20%) as a white solid.

Step 2: Synthesis of 352-2: A solution of NaH (5.88 g, 245.152 mmol, 2 equiv) in THE (200 mL) was treated with 352-1 (27 g, 122.576 mmol, 1 equiv) for 10 min at 0° C. under a nitrogen atmosphere followed by the addition of CD₃I (9.91 mL, 159.349 mmol, 1.3 equiv) dropwise at room temperature. This reaction was stirred at room temperature for 3 h. The reaction was quenched with sat. NH₄Cl (aq.) at room temperature. The resulting mixture was extracted with EtOAc (3×150 mL). The combined organic layers dried over anhydrous MgSO4. After filtration, the filtrate was concentrated under reduced pressure to give 352-2 (28 g, 96.26%) as yellow solid.

Step 3: Synthesis of 353-3: To a solution of 353-2 (5 g, 21.069 mmol, 1 equiv) in MeOH (100 mL) was added Pd/C (6.73 g, 63.207 mmol, 3 equiv) under a nitrogen atmosphere in a 250 mL round-bottom flask. The mixture was hydrogenated at 50° C. overnight under a hydrogen atmosphere using a hydrogen balloon. The mixture of filtered through a celite pad and concentrated under reduced pressure to give 353-3 (3.1 g, 98.61%) as a brown oil. The crude product was used in the next step directly without further purification.

Step 4: Synthesis of 353-4: A solution of 353-3 (3 g, 20.106 mmol, 1 equiv) in HCl(gas) in 1,4-dioxane (50 mL) was stirred overnight at room temperature under a nitrogen atmosphere. The precipitated solids were collected by filtration and washed with acetonitrile (3×10 mL) to give 353-4 (2.45 g, 99.87%) as a white solid. The crude product was used in the next step directly without further purification.

Step 5: Synthesis of 353-5: A solution of ethyl 4,6-dichloro-5-nitropyridine-3-carboxylate (5 g, 18.864 mmol, 1.00 equiv) in EtOH (200 mL) was treated with Et₃N (7.87 mL, 56.592 mmol, 3 equiv) at 0° C. followed by the addition of 353-4 (2.53 g, 20.750 mmol, 1.1 equiv) at 0° C. This reaction was stirred from 0° C. to room temperature for 3 h. The resulting mixture was concentrated under vacuum. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 0% to 100% gradient in 20 min; detector: UV 254 nm to give 353-5 (1.2 g, 27.47%) as a brick red solid.

Step 6: Synthesis of 353-6: A solution of 353-5 (1.1 g, 4.749 mmol, 1 equiv) and CH₃ONa (1.28 g, 23.745 mmol, 5 equiv) in CH₃OH (150 mL) was stirred overnight at 80° C. under a nitrogen atmosphere. The resulting mixture was concentrated under vacuum. The residue was purified by reversed-phase flash chromatography with the following conditions: column: C18 silica gel; mobile phase: MeCN in water, 0% to 50% gradient in 20 min; detector: UV 254 nm to afford 353-6 (1 g, 92.68%) as a red solid.

Step 7: Synthesis of 353-7: A solution of 353-6 (1 g, 4.402 mmol, 1 equiv) in DCM (10 mL) was treated with pyridine (1.39 g, 17.608 mmol, 4 equiv) for 5 min at room temperature followed by the addition of Tf₂O (1.86 mL, 11.005 mmol, 2.5 equiv) dropwise at 0° C. The resulting mixture was stirred from 0° C. to room temperature overnight. The resulting mixture was concentrated under vacuum. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by silica gel column chromatography and eluted with PE/EA (6:1) to afford 353-7 (980 mg, 61.98%) as a yellow solid.

Step 8: Synthesis of 353-8: A solution of 353-7 (1 g, 2.784 mmol, 1 equiv) and Pd/C (1.48 g, 13.920 mmol, 5 equiv) in MeOH (100 mL) was stirred overnight at room temperature under a hydrogen atmosphere. The resulting mixture was filtered and the filter cake was washed with MeOH (3×20 mL). The filtrate was concentrated under reduced pressure. The residue was purified by reversed-phase flash chromatography with the following conditions: column: C18 silica gel; mobile phase: MeCN in water (10 mmol/L NH₄HCO₃), 0% to 100% gradient in 20 min; detector: UV 254 nm to give 353-8 (400 mg, 79.30%) as a brown solid.

Step 9: Synthesis of N-(6-methoxy-1-(methyl-d3)-1H-pyrazolo[4,3-c]pyridin-7-yl)-1-(4-(trifluoromethyl)pyridin-2-yl)-1H-pyrazole-4-sulfonamide (I-352): A solution of 353-8 (400 mg, 2.207 mmol, 1 equiv) and 1-[4-(trifluoromethyl)pyridin-2-yl]pyrazole-4-sulfonyl chloride (1.03 g, 3.310 mmol, 1.5 equiv) in pyridine (20 mL) was stirred overnight at room temperature. The resulting mixture was concentrated under vacuum. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 30% B to 50% B in 7 min; wave length: 220 nm; RT1(min): 7.02) to afford N-(6-methoxy-1-(methyl-d3)-1H-pyrazolo[4,3-c]pyridin-7-yl)-1-(4-(trifluoromethyl)pyridin-2-yl)-1H-pyrazole-4-sulfonamide (I-352, 57.4 mg, 5.68%) as an off-white solid. LCMS (ES, m/z): [M+1]⁺ 457; ¹H NMR (400 MHz, DMSO-d₆) δ 3.45 (s, 3H), 7.87 (dd, J=1.2, 5.2 Hz, 1H), 8.09 (d, J=0.4 Hz, 1H), 8.19 (s, 1H), 8.21 (s, 1H) 8.65 (s, 1H), 8.81 (s, 1H), 8.83 (d, J=6.4 Hz, 1H), 9.95 (s, 1H)

### Example 369—Synthesis of N-(1-(Methyl-D3)-1H-Indazol-7-yl-3-D)-1-(4-(Trifluoromethyl)Pyridin-2-yl)-1H-Pyrazole-4-Sulfonamide (I-353)

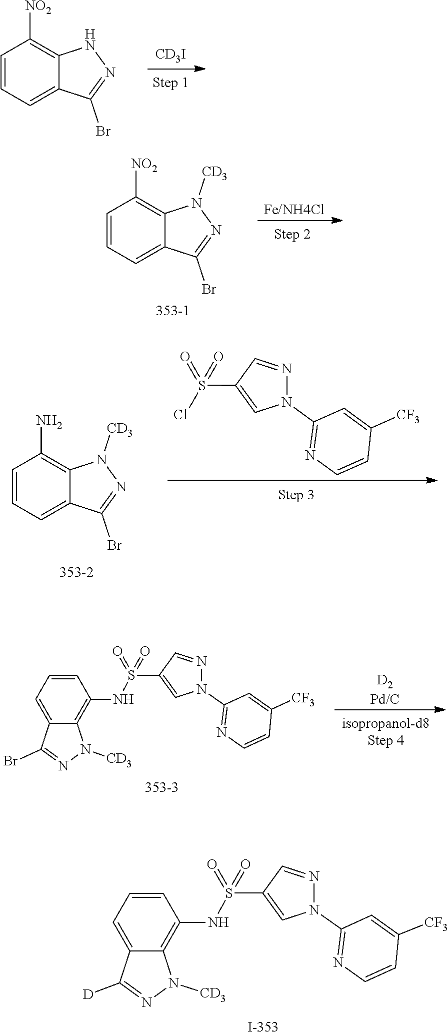

Step 1: Synthesis of 353-1: To a stirred solution of 3-bromo-7-nitro-1H-indazole (1.00 g, 4.132 mmol, 1 equiv) in acetone (10 mL) was added Cs₂CO₃ (2.69 g, 8.264 mmol, 2 equiv) at 0° C. under a nitrogen atmosphere. The resulting mixture was stirred for 30 min at room temperature under a nitrogen atmosphere. To the above mixture was added CD₃I (1.80 g, 12.396 mmol, 3 equiv) dropwise at room temperature. The resulting mixture was stirred for an additional 30 min at room temperature under a nitrogen atmosphere. The reaction was quenched by the addition of sat. NH₄Cl (aq.) (10 mL) at 0° C. The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 60% gradient in 10 min; detector: UV 254 nm. This resulted in 3-bromo-1-(methyl-d₃)-7-nitro-1H-indazole (353-1, 800.00 mg, 67.26%) as a light yellow solid.

Step 2: Synthesis of 353-2: A mixture of 3-bromo-1-(methyl-d₃)-7-nitro-1H-indazole (400.00 mg, 1.544 mmol, 1 equiv), Fe (431.11 mg, 7.720 mmol, 5 equiv) and NH₄Cl (412.93 mg, 7.720 mmol, 5 equiv) in EtOH (40 mL) was stirred for 16 h at 80° C. under a nitrogen atmosphere. The resulting mixture was extracted with EtOAc (3×60 mL). The combined organic layers were washed with brine (1×100 mL) and dried over anhydrous Na₂SO₄. After filtration, the filtrate was concentrated under reduced pressure. The residue was purified by silica gel column chromatography and eluted with PE/EA (1:1) to afford 3-bromo-1-(methyl-d₃)-1H-indazol-7-amine (353-2, 250.00 mg, 68.63%) as an off-white solid.

Step 3: Synthesis of 353-3: A mixture of 1-(4-(trifluoromethyl)pyridin-2-yl)-1H-pyrazole-4-sulfonyl chloride (442.13 mg, 1.418 mmol, 1.3 equiv) and 3-bromo-1-(methyl-d₃)-1H-indazol-7-amine (250 mg, 1.091 mmol, 1.00 equiv) in pyridine (20 mL) was stirred for 30 min at 80° C. The resulting mixture was diluted with water (30 mL) and extracted with EtOAc (3×30 mL). The combined organic layers were washed with brine (1×100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 60% to 70% gradient in 10 min; detector: UV 254 nm, to afford N-(3-bromo-1-(methyl-d₃)-1H-indazol-7-yl)-1-(4-(trifluoromethyl)pyridin-2-yl)-1H-pyrazole-4-sulfonamide (353-3, 300.00 mg, 52.93%) as an off-white solid.

Step 4: Synthesis of N-(1-(methyl-d3)-1H-indazol-7-yl-3-d)-1-(4-(trifluoromethyl)pyridin-2-yl)-1H-pyrazole-4-sulfonamide (I-353): To a solution of N-(3-bromo-1-(methyl-d₃)-1H-indazol-7-yl)-1-(4-(trifluoromethyl)pyridin-2-yl)-1H-pyrazole-4-sulfonamide (350 mg, 0.694 mmol, 1 equiv) in 5 mL of isopropanol was added Pd/C (10%, 7.39 mg) under a nitrogen atmosphere in a 25 mL round-bottom flask. The mixture was hydrogenated at room temperature for 1 h under a hydrogen atmosphere using a hydrogen balloon, filtered through a celite pad and concentrated under reduced pressure. The residue was purified by reversed-phase flash chromatography with the following conditions: column: C18 silica gel; mobile phase: MeCN in water, 60% to 70% gradient in 10 min; detector: UV 254 nm, to afford N-(1-(methyl-d₃)-1H-indazol-7-yl-3-d)-1-(4-(trifluoromethyl)pyridin-2-yl)-1H-pyrazole-4-sulfonamide (I-353, 155.2 mg, 52.29%) as an off-white solid. LCMS (ES, m/z): [M+H]⁺=427; ¹H NMR: (400 MHz, DMSO-d₆) δ 6.73 (dd, J=1.2, 7.2 Hz, 1H), 6.97 (t, J=8.0 Hz, 1H), 7.71 (dd, J=1.2, 8.0 Hz, 1H), 7.89 (dd, J=1.6, 5.2 Hz, 1H), 8.10 (s, 1H), 8.20 (s, 1H), 8.82 (d, J=5.2 Hz, 1H), 8.87 (s, 1H), 10.08 (s, 1H).

### Example 370—Synthesis of N-(6-Methoxy-1-(Methyl-D3)-1H-Pyrazolo[4,3-C]Pyridin-7-yl)-N-Methyl-1-(4-(Trifluoromethyl) Pyridin-2-yl)-1H-Pyrazole-4-Sulfonamide (I-354)

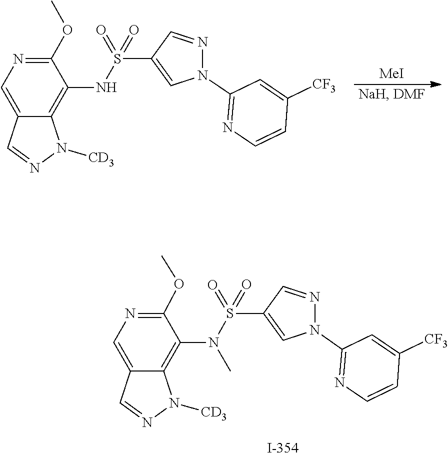

To a solution of N-(6-methoxy-1-(methyl-d3)-1H-pyrazolo[4,3-c]pyridin-7-yl)-1-(4-(trifluoromethyl)pyridin-2-yl)-1H-pyrazole-4-sulfonamide (100 mg, 0.219 mmol, 1 equiv) in DMF (3 mL) was added sodium hydride (60% in oil, 15.77 mg) at 0° C. The mixture was stirred for 15 min. Mel (0.02 mL, 0.329 mmol, 1.5 equiv) was added and the mixture was allowed to warm to rt and stirred for 3 h. This reaction was allowed to stir for another 3 h at room temperature. The reaction was quenched by the addition of sat. NH₄Cl (aq.) (20 mL) at room temperature. The resulting mixture was extracted with CH₂Cl₂ (3×20 mL). The combined organic layers were washed with sat. NaCl(aq) (1×50 mL) and dried over anhydrous Na₂SO₄. After filtration, the filtrate was concentrated under reduced pressure. The crude product (150 mg) was purified by Prep-HPLC with the following conditions (column: XBridge Prep OBD C18 column, 30*150 mm, 5 μm; mobile phase A: water (10 mmol/L NH₄HCO₃+0.05% NH₃·H₂O), mobile phase B: ACN; flow rate: 60 mL/min; gradient: 45% B to 67% B in 7 min; wave length: 254 nm/220 nm; RT1(min): 7.08; number of runs: 3) to afford N-(6-methoxy-1-(methyl-d3)-1H-pyrazolo[4,3-c]pyridin-7-yl)-N-methyl-1-(4-(trifluoromethyl)pyridin-2-yl)-1H-pyrazole-4-sulfonamide (I-354, 51.8 mg, 50.10%) as an off-white solid. LCMS (ES, m/z): [M+H]⁺=471; ¹H NMR: (400 MHz, CD3CN) δ 3.33 (s, 3H), 3.58 (s, 3H), 7.70 (d, J=4.8 Hz, 1H), 8.03 (s, 1H), 8.11 (s, 1H), 8.28 (s, 1H), 8.66 (s, 1H), 8.74 (d, J=5.2 Hz, 1H), 8.92 (s, 1H).

### Example 371—Synthesis of 1-((1-(4-(Trifluoromethyl)Pyridin-2-yl)-1H-Pyrazol-4-yl)Sulfonyl)-2,3-Dihydro-1H-Pyrazolo[1,5,4-De]Quinoxaline (I-355)

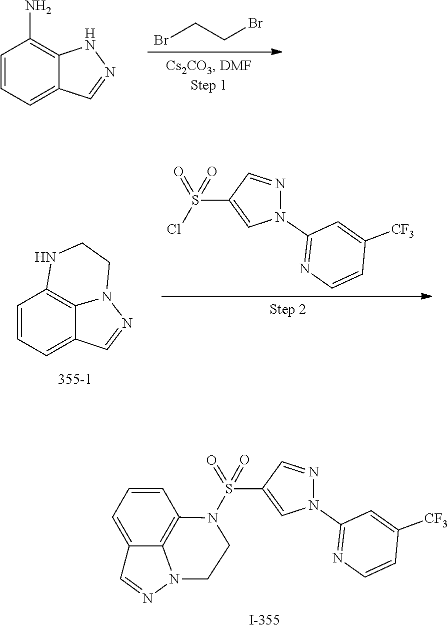

Step 1: Synthesis of 355-1: A solution of 1H-indazol-7-amine (1 g, 7.510 mmol, 1 equiv) and dibromoethane (1.55 g, 8.261 mmol, 1.1 equiv) and Cs₂CO₃ (6.12 g, 18.775 mmol, 2.5 equiv) in DMF (10 mL) was stirred overnight at room temperature under a nitrogen atmosphere. The resulting mixture was diluted with water (50 mL). The resulting mixture was extracted with EtOAc (3×100 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: ACN in water (10 mmol/L NH₄HCO₃), 30% to 50% gradient in 10 min; detector: UV 254 nm. This resulted in 355-1 (380 mg, 31.78%) as a yellow solid.

Step 2: Synthesis of 1-((1-(4-(trifluoromethyl)pyridin-2-yl)-1H-pyrazol-4-yl)sulfonyl)-2,3-dihydro-1H-pyrazolo[1,5,4-de]quinoxaline (I-355): A solution of 355-1 (89 mg, 0.559 mmol, 1 equiv) and 1-[4-(trifluoromethyl)pyridin-2-yl]pyrazole-4-sulfonyl chloride (209.09 mg, 0.671 mmol, 1.2 equiv) in pyridine (10 mL) was stirred overnight at room temperature. After the reaction was complete, the resulting mixture was concentrated under vacuum. The residue was purified by reversed-phase flash chromatography with the following conditions: column: C₁₈ silica gel; mobile phase: ACN in water (10 mmol/L NH₄HCO₃), 0% to 100% gradient in 10 min; detector: UV 254 nm to give the crude product. The crude product (150 mg) was purified by Prep-HPLC with the following conditions (column: XBridge Prep OBD C₁₈ column, 30*150 mm, 5 μm; mobile phase A: water (10 mmol/L NH₄HCO₃+0.05% NH₃H₂O), mobile phase B: ACN; flow rate: 60 mL/min; gradient: 43% B to 68% B in 7 min; wave length: 220 nm; RT₁(min): 7.65) to afford 1-((1-(4-(trifluoromethyl)pyridin-2-yl)-1H-pyrazol-4-yl)sulfonyl)-2,3-dihydro-1H-pyrazolo[1,5,4-de]quinoxaline (I-355, 53.2 mg, 20.92%) as an off-white solid. LCMS (ES, m/z): [M+1]⁺=435; ¹H NMR: (400 MHz, DMSO-d₆) δ 4.33 (d, J=5.2 Hz, 2H), 4.43 (t, J=4.0 Hz, 2H), 7.17 (t, J=8.0 Hz, 1H), 7.51-7.58 (m, 2H), 7.86 (d, J=4.8 Hz, 1H), 8.04 (s, 1H), 8.09 (s, 1H), 8.26 (s, 1H) 8.79 (d, J=5.2 Hz, 1H), 9.21 (s, 1H).

### Example 372—Synthesis of 1-(4-(Trifluoromethyl)Pyridin-2-yl)-N-(2-Vinyl-2H-Indazol-7-yl)-1H-Pyrazole-4-Sulfonamide (I-357)

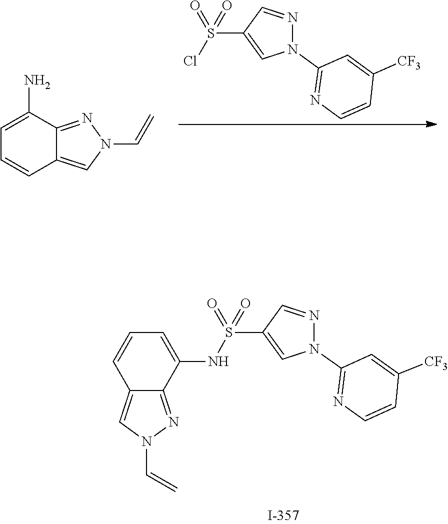 [SMILES: C=Cn1cc2cccc(N)c2n1 ; O=S(=O)(Cl)c1cnn(-c2cc(C(F)(F)F)ccn2)c1 ; C=Cn1cc2cccc(NS(=O)(=O)c3cnn(-c4cc(C(F)(F)F)ccn4)c3)c2n1]

A solution of 2-ethenylindazol-7-amine (89 mg, 0.559 mmol, 1 equiv) and 1-[4-(trifluoromethyl)pyridin-2-yl]pyrazole-4-sulfonyl chloride (209.09 mg, 0.671 mmol, 1.2 equiv) in pyridine (10 mL) was stirred overnight at room temperature. After the reaction was complete, the resulting mixture was concentrated under vacuum. The residue was purified by reversed-phase flash chromatography with the following conditions: column: C₁₈ silica gel; mobile phase: ACN in water (10 mmol/L NH₄HCO₃), 0% to 100% gradient in 10 min; detector: UV 254 nm to give the crude product. The crude product (150 mg) was purified by Prep-HPLC with the following conditions (column: XBridge Prep OBD C₁₈ column, 30*150 mm, 5 μm; mobile phase A: water (10 mmol/L NH₄HCO₃+0.05% NH₃H₂O), mobile phase B: ACN; flow rate: 60 mL/min; gradient: 43% B to 68% B in 7 min; wave length: 220 nm; RT₁(min): 7.65) to afford 1-(4-(trifluoromethyl)pyridin-2-yl)-N-(2-vinyl-2H-indazol-7-yl)-1H-pyrazole-4-sulfonamide (I-357, 49.9 mg, 20.55%) as an off-white solid. LCMS (ES, m/z): [M+1]⁺=435; ¹H NMR: (400 MHz, DMSO-d₆) δ 5.18 (d, J=8.4 Hz, 1H), 6.03 (d, J=12.8 Hz, 1H), 7.02 (t, J=8.4 Hz, 1H), 7.23 (d, J=7.2 Hz, 1H), 7.48-7.54 (m, 2H), 7.83 (d, J=4.8 Hz, 1H), 8.09 (s, 1H), 8.22 (d, J=12.8 Hz, 1H), 8.63 (s, 1H), 8.78 (d, J=4.8 Hz, 1H), 9.13 (s, 1H), 10.42 (s, 1H).

### Example 373—Synthesis of N-(6-Methoxy-1-Methyl-1H-Indazol-7-yl)-1-(4-(2-Methoxybutan-2-yl)Pyridin-2-yl)-1H-Pyrazole-4-Sulfonamide (I-360)

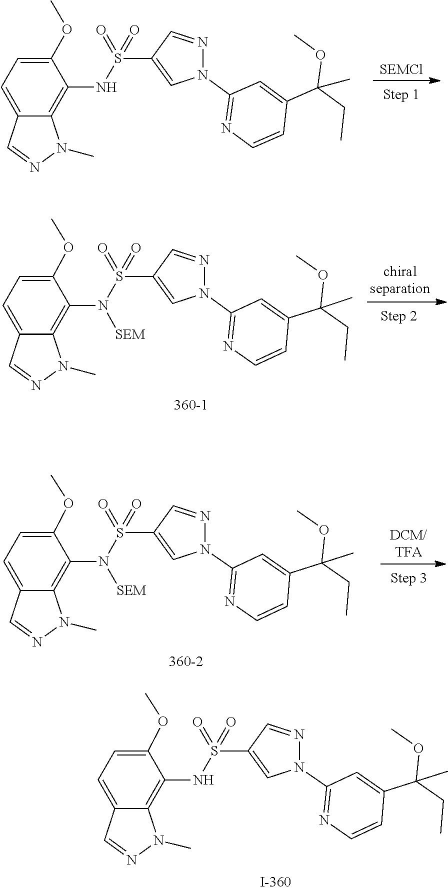

Step 1: Synthesis of 360-1: To a stirred solution of N-(6-methoxy-1-methylindazol-7-yl)-1H-pyrazole-4-sulfonamide (68 mg, 0.221 mmol, 1 equiv) and SEM-C1 (36.89 mg, 0.221 mmol, 1 equiv) in DMF (1 mL) was added TEA (67.17 mg, 0.663 mmol, 3 equiv) dropwise at room temperature under a nitrogen atmosphere. The resulting mixture was stirred for 1 h at room temperature under a nitrogen atmosphere. The resulting mixture was diluted with water (10 mL) and extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: MeCN in water (0.1% FA), 0% to 100% gradient in 20 min; detector: UV 254 nm. The resulting mixture was concentrated under vacuum. This resulted in N-(6-methoxy-1-methylindazol-7-yl)-1-[4-(2-methoxybutan-2-yl)pyridin-2-yl]-N-{[2-(trimethylsilyl)ethoxy]methyl}pyrazole-4-sulfonamide (360-1, 100 mg, 103.66%) as a yellow oil.

Step 2: Synthesis of 360-2: The crude product 360-1 (120 mg crude) was purified by chiral HPLC with the following conditions (column: CHIRALPAKIF-34.6*50 mm, 3.0 um; mobile phase A: hex(0.2% DEA): (EtOH:DCM=1:1)=80:20; gradient: isocratic; injection volume: 3.0 mL) to afford 360-2 (30 mg, 25%) as a white solid.

Step 3: Synthesis of N-(6-methoxy-1-methyl-1H-indazol-7-yl)-1-(4-(2-methoxybutan-2-yl)pyridin-2-yl)-1H-pyrazole-4-sulfonamide (I-360): A solution of (S)—N-(6-methoxy-1-methyl-1H-indazol-7-yl)-1-(4-(2-methoxybutan-2-yl)pyridin-2-yl)-N-((2-(trimethylsilyl)ethoxy)methyl)-1H-pyrazole-4-sulfonamide (30 mg, 0.050 mmol, 1 equiv) in TFA (1 mL) and DCM (1 mL) was stirred at room temperature under a nitrogen atmosphere. The resulting mixture was stirred for 2 h at room temperature under a nitrogen atmosphere. The resulting mixture was concentrated under vacuum. The residue was purified by reversed-phase flash chromatography with the following conditions: column: C₁₈ silica gel; mobile phase: MeCN in water (0.1% FA), 0% to 100% gradient in 20 min; detector: UV 254 nm. This resulted in N-(6-methoxy-1-methyl-1H-indazol-7-yl)-1-(4-(2-methoxybutan-2-yl)pyridin-2-yl)-1H-pyrazole-4-sulfonamide as a single steroeoisomer (I-360, 5 mg, 21.24%) as a white solid. LCMS (ES, m/z): [M+H]⁺ 471; ¹H NMR: (400 MHz, methanol-d₄) δ 0.68 (t, J=7.4 Hz, 3H), 1.46 (s, 3H), 1.64-1.87 (m, 2H), 3.10 (s, 3H), 3.30 (s, 3H), 4.25 (s, 3H), 6.75 (d, J=8.8 Hz, 1H), 7.30 (dd, J=1.6, 5.2 Hz, 1H), 7.57 (d, J=8.8 Hz, 1H), 7.74 (d, J=0.8 Hz, 1H), 7.84 (s, 1H), 7.91 (dd, J=0.8, 1.6 Hz, 1H), 8.31 (dd, J=0.8, 5.2 Hz, 1H), 8.64 (d, J=0.8 Hz, 1H).

### Example 374—Synthesis of N-(6-Methoxy-1-Methyl-1H-Indazol-7-yl)-1-(4-(2-Methoxybutan-2-yl)Pyridin-2-yl)-1H-Pyrazole-4-Sulfonamide

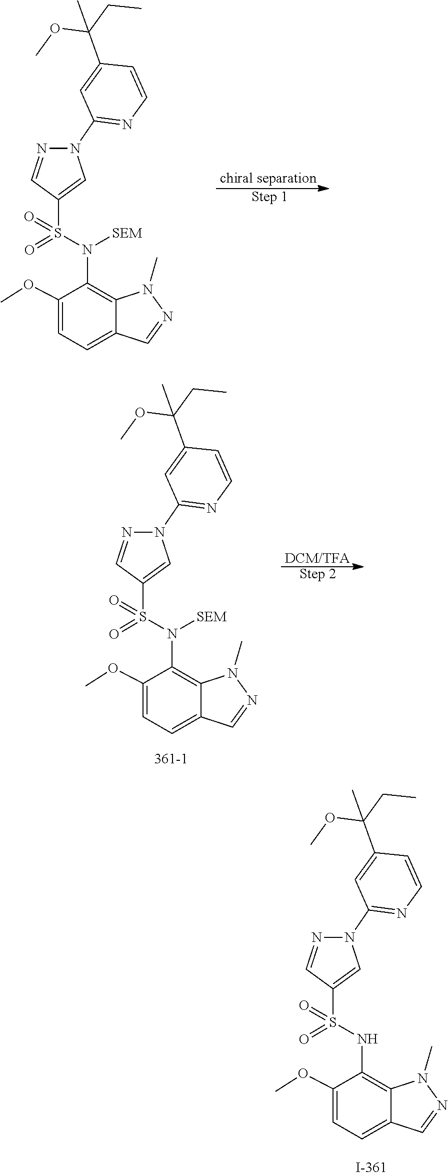

Step 1: Synthesis of 360-1: The crude product N-(6-methoxy-1-methyl-1H-indazol-7-yl)-1-(4-(2-methoxybutan-2-yl)pyridin-2-yl)-N-((2-(trimethylsilyl)ethoxy)methyl)-1H-pyrazole-4-sulfonamide (120 mg crude) was purified by chiral HPLC with the following conditions (column: CHIRALPAKIF-34.6*50 mm, 3.0 μm; mobile phase A: hex(0.2% DEA): (EtOH: DCM=1:1)=80:20; gradient: isocratic; injection volume: 3.0 mL) to afford 361-1 (52 mg, 46.8%) as a white solid.

Step 2: Synthesis of N-(6-methoxy-1-methyl-1H-indazol-7-yl)-1-(4-(2-methoxybutan-2-yl)pyridin-2-yl)-1H-pyrazole-4-sulfonamide (I-361): To a stirred solution of (R)—N-(6-methoxy-1-methyl-1H-indazol-7-yl)-1-(4-(2-methoxybutan-2-yl)pyridin-2-yl)-N-((2-(trimethylsilyl)ethoxy)methyl)-1H-pyrazole-4-sulfonamide (52 mg, 0.087 mmol, 1 equiv) in TFA (1 mL) and DCM (1 mL) was stirred at room temperature under a nitrogen atmosphere. The resulting mixture was stirred for 2 h at room temperature under a nitrogen atmosphere. The resulting mixture was concentrated under vacuum. The residue was purified by reversed-phase flash chromatography with the following conditions: column: Cis silica gel; mobile phase: MeCN in water (0.1% FA), 0% to 100% gradient in 20 min; detector: UV 254 nm. This resulted in N-(6-methoxy-1-methyl-1H-indazol-7-yl)-1-(4-(2-methoxybutan-2-yl)pyridin-2-yl)-1H-pyrazole-4-sulfonamide as a single steroeoisomer (I-361, 7.1 mg, 17.40%) as a white solid. LCMS (ES, m/z): [M+H]7471; ¹H NMR: (400 MHz, methanol-d₄) δ 0.68 (t, J=7.2 Hz, 3H), 1.46 (s, 3H), 1.64-1.87 (m, 2H), 3.10 (s, 3H), 3.30 (s, 3H), 4.25 (s, 3H), 6.75 (d, J=8.8 Hz, 1H), 7.30 (dd, J=1.6, 5.2 Hz, 1H), 7.57 (d, J=8.8 Hz, 1H), 7.74 (d, J=0.8 Hz, 1H), 7.84 (s, 1H), 7.91 (dd, J=0.8, 1.6 Hz, 1H), 8.31 (dd, J=0.8, 5.2 Hz, 1H), 8.64 (d, J=0.8 Hz, 1H).

### Example 375—Synthesis of N-(6-Methoxy-1-Methyl-1H-Indazol-7-yl)-1-(4-(1-Methoxy-3-Methylcyclobutyl)Pyridin-2-yl)-1H-Pyrazole-4-Sulfonamide (I-364)

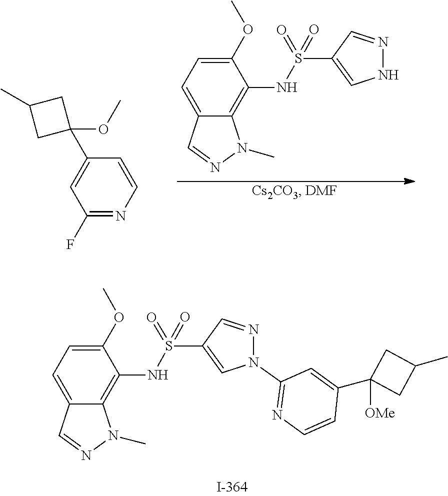 [SMILES: COC1(c2ccnc(F)c2)CC(C)C1 ; COc1ccc2cnn(C)c2c1NS(=O)(=O)c1cn[nH]c1 ; COc1ccc2cnn(C)c2c1NS(=O)(=O)c1cnn(-c2cc(C3(OC)CC(C)C3)ccn2)c1]

A solution of 2-fluoro-4-[1-methoxy-3-methylcyclobutyl]pyridine (100 mg, 0.512 mmol, 1 equiv), N-(6-methoxy-1-methylindazol-7-yl)-1H-pyrazole-4-sulfonamide (188.90 mg, 0.614 mmol, 1.2 equiv) and Cs₂CO₃ (500.65 mg, 1.536 mmol, 3 equiv) in DMF (3 mL) was stirred overnight at 100° C. The mixture was allowed to cool down to room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by Prep-HPLC with the following conditions (column: X select CSH C18 OBD column 30*150 mm 6 m; mobile phase A: water(0.1% FA), mobile phase B: ACN; flow rate: 60 mL/min; gradient: 46% B to 60% B in 7 min; wave length: 254 nm/220 nm; RT1 (min): 6.7) to afford N-(6-methoxy-1-methyl-1H-indazol-7-yl)-1-(4-(1-methoxy-3-methylcyclobutyl)pyridin-2-yl)-1H-pyrazole-4-sulfonamide as a single steroeoisomer (I-364, 43 mg, 17.26%) as an off-white solid. LCMS (ES, m/z): [M+H]⁺ 483; ¹H NMR: (400 MHz, methanol-d₄) δ 1.13 (d, J=6.2 Hz, 3H), 1.95-2.06 (m, 2H), 2.55-2.71 (m, 3H), 3.05 (s, 3H), 3.40 (s, 3H), 4.34 (s, 3H), 6.84 (d, J=8.8 Hz, 1H), 7.36 (dd, J=1.6, 5.2 Hz, 1H), 7.67 (d, J=8.8 Hz, 1H), 7.85 (s, 1H), 7.94 (s, 2H), 8.44 (d, J=5.2 Hz, 1H), 8.74 (s, 1H).

### Example 376—Synthesis of N-(6-Methoxy-1-Methyl-1H-Indazol-7-yl)-1-(4-((1-Methoxy-3-Methylcyclobutyl)Pyridin-2-yl)-1H-Pyrazole-4-Sulfonamide (I-365)

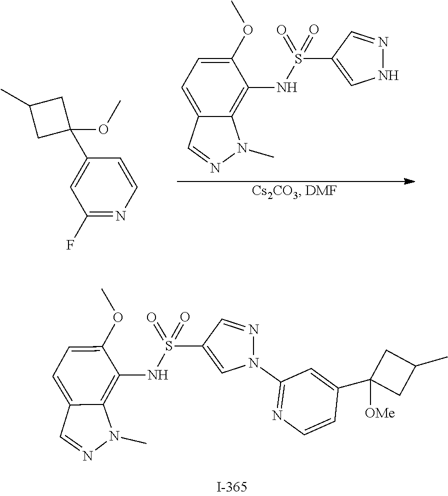 [SMILES: COC1(c2ccnc(F)c2)CC(C)C1 ; COc1ccc2cnn(C)c2c1NS(=O)(=O)c1cn[nH]c1 ; COc1ccc2cnn(C)c2c1NS(=O)(=O)c1cnn(-c2cc(C3(OC)CC(C)C3)ccn2)c1]

A solution of 2-fluoro-4-[1-methoxy-3-methylcyclobutyl]pyridine (100 mg, 0.512 mmol, 1 equiv), N-(6-methoxy-1-methylindazol-7-yl)-1H-pyrazole-4-sulfonamide (188.90 mg, 0.614 mmol, 1.2 equiv) and Cs₂CO₃ (500.65 mg, 1.536 mmol, 3 equiv) in DMF (3 mL) was stirred overnight at 100° C. under a nitrogen atmosphere. The mixture was allowed to cool down to room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by Prep-HPLC with the following conditions (column: XBridge Prep OBD C18 column, 30*150 mm, 5 μm; mobile phase A: water (10 mmol/L NH₄HCO₃)+0.05% NH₃·H₂O, mobile phase B: ACN; flow rate: 60 mL/min; gradient: 36% B to 63% B in 7 min; wave length: 254 nm/220 nm; RT1(min): 7.7) to afford N-(6-methoxy-1-methyl-1H-indazol-7-yl)-1-(4-(1-methoxy-3-methylcyclobutyl)pyridin-2-yl)-1H-pyrazole-4-sulfonamide as a single steroeoisomer (I-365, 52.5 mg, 21.16%) as an off-white solid. LCMS (ES, m/z): [M+H]⁺ 483; ¹H NMR: (400 MHz, methanol-d₄) δ 1.20 (d, J=6.4 Hz, 3H), 1.99-2.10 (m, 3H), 2.07-2.18 (m, 1H), 2.54-2.64 (m, 2H), 3.00 (s, 3H), 3.40 (s, 3H), 4.34 (s, 3H), 6.84 (d, J=8.8 Hz, 1H), 7.48 (dd, J=1.6, 5.2 Hz, 1H), 7.66 (d, J=8.8 Hz, 1H), 7.85 (d, J=0.8 Hz, 1H), 7.94 (s, 1H), 8.06 (dd, J=0.8, 1.6 Hz, 1H), 8.46 (dd, J=0.8, 5.2 Hz, 1H), 8.75 (d, J=0.8 Hz, 1H).

### Example 377—Synthesis of 1-(4-(5-Azaspiro[2.3]Hexan-5-yl)Pyridin-2-yl)-N-(1-Methyl-1H-Indazol-7-yl)-1H-Pyrazole-4-Sulfonamide (I-368)

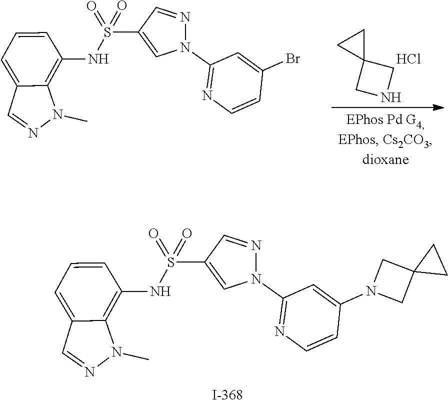 [SMILES: Cn1ncc2cccc(NS(=O)(=O)c3cnn(-c4cc(Br)ccn4)c3)c21 ; C1CC12CNC2 ; Cl ; Cn1ncc2cccc(NS(=O)(=O)c3cnn(-c4cc(N5CC6(CC6)C5)ccn4)c3)c21]

A solution of 1-(4-bromopyridin-2-yl)-N-(1-methylindazol-7-yl)pyrazole-4-sulfonamide (100 mg, 0.231 mmol, 1 equiv), 5-azaspiro[2.3]hexane hydrochloride (138.01 mg, 1.155 mmol, 5 equiv), EPhos (12.34 mg, 0.023 mmol, 0.1 equiv), EPhos Pd G4 (21.20 mg, 0.023 mmol, 0.1 equiv) and Cs₂CO₃ (225.59 mg, 0.693 mmol, 3 equiv) in 1,4-dioxane (8 mL) was stirred overnight at 100° C. under a nitrogen atmosphere. The reaction was quenched with water at room temperature. The resulting mixture was extracted with EtOAc (2×50 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 1-(4-{5-azaspiro[2.3]hexan-5-yl}pyridin-2-yl)-N-(1-methylindazol-7-yl)pyrazole-4-sulfonamide) (I-368) as a white solid. LCMS (ES, m/z): [M+H]=436; ¹H NMR: (400 MHz, DMSO-d₆): δ 0.72 (m, 4H), 4.10 (s, 4H), 4.28 (s, 3H), 6.41 (dd, J=5.8, 2.0 Hz, 1H), 6.69 (d, J=7.4 Hz, 1H), 6.85 (d, J=2.2 Hz, 1H), 6.98 (t, J=7.6 Hz, 1H), 7.69 (d, J=8.0 Hz, 1H), 7.93 (s, 1H), 8.01-8.10 (m, 2H), 8.71 (s, 1H), 9.97 (s, 1H).

### Example 378—Synthesis of 1-(4-(Azetidin-1-yl)Pyridin-2-yl)-N-(6-Methoxy-1-Methyl-1H-Indazol-7-yl)-1H-Pyrazole-4-Sulfonamide (I-373)

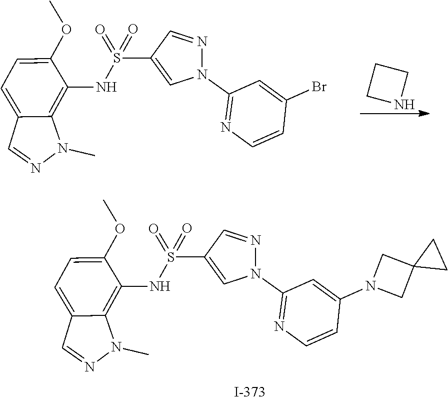

To a stirred mixture of 1-(4-bromopyridin-2-yl)-N-(6-methoxy-1-methylindazol-7-yl)pyrazole-4-sulfonamide (150 mg, 0.324 mmol, 1 equiv) and Cs₂CO₃ (316.46 mg, 0.972 mmol, 3 equiv) in dioxane (10 mL) were added azetidine (36.97 mg, 0.648 mmol, 2 equiv) EPhos Pd G4 (29.74 mg, 0.032 mmol, 0.1 equiv) and EPhos (17.31 mg, 0.032 mmol, 0.1 equiv) at 100° C. under a nitrogen atmosphere. The resulting mixture was stirred for additional 2 h at 100° C. The crude product (150 mg) was purified by Prep-HPLC with the following conditions (column: XBridge Prep OBD C18 column, 30*150 mm, 5 μm; mobile phase A: water (10 mmol/L NH₄HCO₃)+0.05% NH₃·H₂O, mobile phase B: ACN; flow rate: 60 mL/min; gradient: 33% B to 60% B in 7 min; wave length: 254 nm/220 nm; RT1(min): 7.8) to afford 1-(4-(azetidin-1-yl)pyridin-2-yl)-N-(6-methoxy-1-methyl-1H-indazol-7-yl)-1H-pyrazole-4-sulfonamide (I-373, 20.6 mg, 14.01%) as a light yellow solid. LCMS (ES, m/z): [M+1]⁺=440; ¹H NMR: ¹H NMR (400 MHz, DMSO-d₆) δ 2.38-2.45 (m, 2H), 3.32 (s, 3H), 4.02 (t, J=7.4 Hz, 4H), 4.24 (s, 3H), 6.37 (dd, J=5.8, 2.2 Hz, 1H), 6.79 (d, J=2.2 Hz, 1H), 6.87 (d, J=8.8 Hz, 1H), 7.68 (d, J=8.8 Hz, 1H), 7.89 (s, 1H), 7.97-8.04 (m, 2H), 8.62 (s, 1H).

### Example 379—Synthesis of 1-(4-(3-Hydroxycyclobutyl)Pyridin-2-yl)-N-(1-Methyl-1H-Indazol-7-yl)-1H-Pyrazole-4-Sulfonamide (I-374)

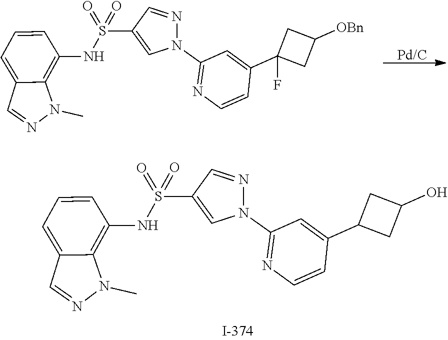 [SMILES: Cn1ncc2cccc(NS(=O)(=O)c3cnn(-c4cc(C5(F)CC(OCc6ccccc6)C5)ccn4)c3)c21 ; Cn1ncc2cccc(NS(=O)(=O)c3cnn(-c4cc(C5CC(O)C5)ccn4)c3)c21]

To a solution of 1-{4-[3-(benzyloxy)-1-fluorocyclobutyl]pyridin-2-yl}-N-(1-methylindazol-7-yl)pyrazole-4-sulfonamide (100 mg, 0.188 mmol, 1 equiv) in 5 mL of EtOAc was added Pd/C (10%, 0.2 g) under a nitrogen atmosphere in a 25 mL round-bottom flask. The mixture was hydrogenated at room temperature for 2 h under a hydrogen atmosphere using a hydrogen balloon. The resulting mixture was filtered and the filter cake was washed with EtOAc (3×100 mL). The filtrate was concentrated under reduced pressure. The crude product (60 mg) was purified by Prep-achiral-SFC with the following conditions (column: GreenSep Naphthyl, 3*25 cm, 5 μm; mobile phase A: CO₂, mobile phase B: MeOH(0.1% 2 M NH₃-MeOH); flow rate: 75 mL/min; gradient: isocratic 25% B; column temperature (° C.): 35; back pressure (bar): 100; wave length: 254 nm; RT1(min): 5.75; sample solvent: MeOH; injection volume: 2.8 mL; number of runs: 5.0) to afford 1-(4-(3-hydroxycyclobutyl)pyridin-2-yl)-N-(1-methyl-1H-indazol-7-yl)-1H-pyrazole-4-sulfonamide (I-374, 14 mg, 17.50%) as a white solid. LCMS (ES, m/z): [M+H]⁺=425; ¹H NMR: (400 MHz, DMSO-d₆) δ 2.31-2.51 (m, 4H), 3.64 (m, J=9.8, 5.4 Hz, 1H), 4.28 (s, 3H), 4.28-4.42 (m, 1H), 5.22 (d, J=5.6 Hz, 1H), 6.70 (m, J=7.6, 1.0 Hz, 1H), 6.98 (t, J=7.6 Hz, 1H), 7.31-7.42 (m, 1H), 7.70 (d, J=8.0 Hz, 1H), 7.83 (s, 1H), 8.01 (s, 1H), 8.09 (s, 1H), 8.41 (m, 1H), 8.79 (d, J=1.6 Hz, 1H), 10.02 (s, 1H).

### Example 380—Synthesis of 1-(4-(3-Azabicyclo[3.1.0]Hexan-3-yl)Pyridin-2-yl)-N-(1-Methyl-1H-Indazol-7-yl)-1H-Pyrazole-4-Sulfonamide (I-375)

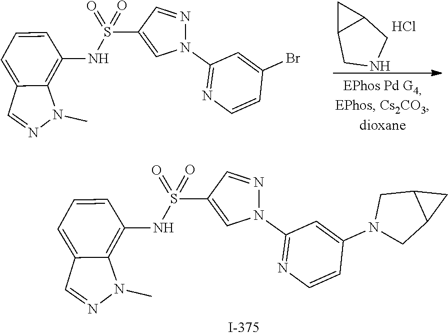 [SMILES: Cn1ncc2cccc(NS(=O)(=O)c3cnn(-c4cc(Br)ccn4)c3)c21 ; C1NCC2CC12 ; Cl ; Cn1ncc2cccc(NS(=O)(=O)c3cnn(-c4cc(N5CC6CC6C5)ccn4)c3)c21]

To a stirred solution of 1-(4-bromopyridin-2-yl)-N-(1-methylindazol-7-yl) pyrazole-4-sulfonamide (100 mg, 0.231 mmol, 1 equiv) and 3-azabicyclo [3.1.0] hexan-3-ium chloride (55.20 mg, 0.462 mmol, 2 equiv) in dioxane (2 mL) were added EPhos (12.34 mg, 0.023 mmol, 0.1 equiv), Cs₂CO₃ (225.59 mg, 0.693 mmol, 3 equiv) and EPhos Pd G4 (21.20 mg, 0.023 mmol, 0.1 equiv) in portions at room temperature under a nitrogen atmosphere. The resulting mixture was stirred for 4 h at 100° C. under a nitrogen atmosphere. The resulting mixture was diluted with water (20 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 1-(4-{3-azabicyclo [3.1.0] hexan-3-yl pyridin-2-yl)-N-(1-methylindazol-7-yl) pyrazole-4-sulfonamide (I-375, 7.3 mg, 7.25%) as a white solid. LCMS (ES, m/z): [M+H]⁺=436; ¹H NMR: (400 MHz, DMSO-d₆) δ 0.19 (m, J=4.4 Hz, 1H), 0.78-0.84 (m, 1H), 1.73-1.78 (m, 2H), 3.41 (m, 2H), 3.57 (d, J=12.0 Hz, 2H), 4.27 (s, 3H), 6.52-6.56 (m, 1H), 6.68 (d, J=8 Hz, 1H), 6.94-7.00 (m, 2H), 7.69 (s, 1H), 7.94 (s, 1H), 8.00 (d, J=8.0 Hz, 1H), 8.08 (s, 1H), 8.70 (s, 1H), 9.98 (s, 1H).

### Example 381—Synthesis of 1-(4-(1,3-Difluorocyclobutyl) Pyridin-2-yl)-N-(6-Methoxy-1-Methyl-1H-Indazol-7-yl)-1H-Pyrazole-4-Sulfonamide (I-377)

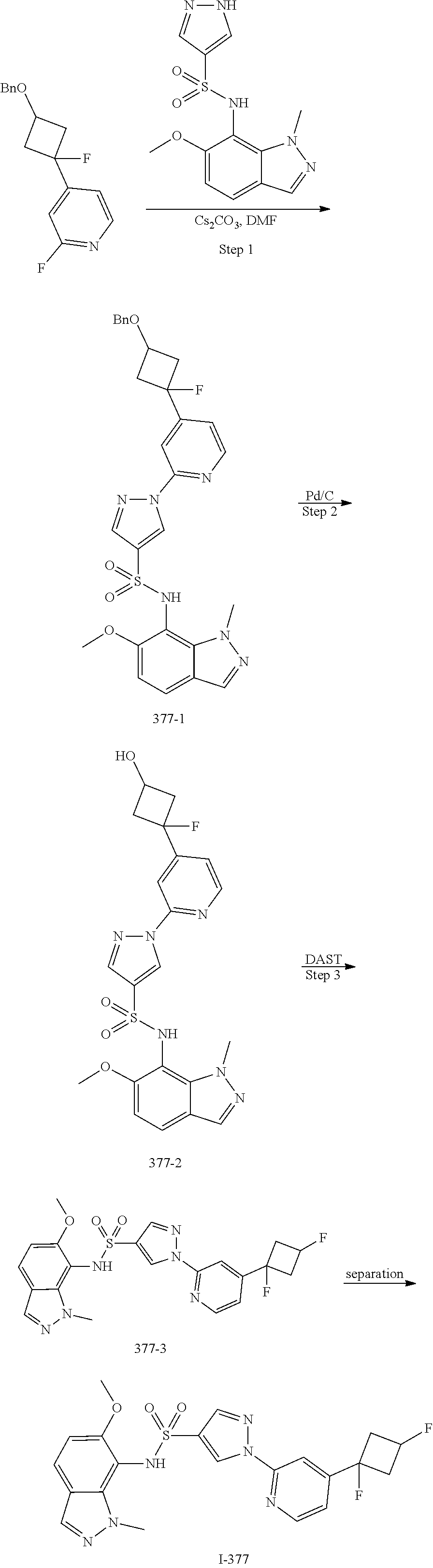

Step 1: Synthesis of 377-1: To a stirred solution of 4-[3-(benzyloxy)-1-fluorocyclobutyl]-2-fluoropyridine (700 mg, 2.543 mmol, 1 equiv) and N-(6-methoxy-1-methylindazol-7-yl)-1H-pyrazole-4-sulfonamide (1172.17 mg, 3.815 mmol, 1.5 equiv) in DMF (12 mL) was added Cs₂CO₃ (2485.37 mg, 7.629 mmol, 3 equiv) in portions at room temperature under a nitrogen atmosphere. The resulting mixture was stirred for 16 h at 80° C. under a nitrogen atmosphere. The resulting mixture was diluted with water (50 mL). The resulting mixture was extracted with EtOAc (3×100 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 1-{4-[3-(benzyloxy)-1-fluorocyclobutyl] pyridin-2-yl}-N-(6-methoxy-1-methylindazol-7-yl) pyrazole-4-sulfonamide (377-1, 770 mg, 53.82%) as a yellow oil.

Step 2: Synthesis of 377-2: To a solution of 1-{4-[3-(benzyloxy)-1-fluorocyclobutyl]pyridin-2-yl}-N-(6-methoxy-1-methylindazol-7-yl) pyrazole-4-sulfonamide (770 mg, 1.369 mmol, 1 equiv) in 15 mL of THF was added Pd/C (10%, 14.56 mg) under a nitrogen atmosphere in a 25 mL pressure tank reactor. The mixture was hydrogenated at room temperature for 2 h under a hydrogen atmosphere using a hydrogen balloon. The resulting mixture was filtered and the filter cake was washed with EtOAc (3×100 mL). The filtrate was concentrated under reduced pressure. The residue was purified by reversed-phase flash chromatography with the following conditions: column: C18 silica gel; mobile phase: MeCN in water (0.1% FA), 60% to 70% gradient in 10 min; detector: UV 254 nm. The resulting mixture was concentrated under reduced pressure. This resulted in 1-[4-(1-fluoro-3-hydroxycyclobutyl) pyridin-2-yl]-N-(6-methoxy-1-methylindazol-7-yl) pyrazole-4-sulfonamide (377-2, 120 mg, 18.56%) as a brown solid.

Step 3: Synthesis of 377-3: To a stirred solution of 1-[4-(1-fluoro-3-hydroxycyclobutyl) pyridin-2-yl]-N-(6-methoxy-1-methylindazol-7-yl) pyrazole-4-sulfonamide (120 mg, 0.253 mmol, 1 equiv) in DCM (3 mL) was added DAST (0.12 mL, 0.761 mmol, 3 equiv) in portions at −78° C. under a nitrogen atmosphere. The resulting mixture was stirred for 1 h at −78° C. under a nitrogen atmosphere. The reaction was quenched by the addition of sat. NaHCO₃ (aq.) (50 mL) at 0° C. The resulting mixture was extracted with EtOAc (3×20 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 1-[4-(1,3-difluorocyclobutyl) pyridin-2-yl]-N-(6-methoxy-1-methylindazol-7-yl) pyrazole-4-sulfonamide (377-3, 52 mg, 43.11%) as an off-white solid.

Step 4: Synthesis of 1-(4-((1,3-difluorocyclobutyl)pyridin-2-yl)-N-(6-methoxy-1-methyl-1H-indazol-7-yl)-1H-pyrazole-4-sulfonamide (I-377): 377-3 (52 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 25 mL/min; gradient: 36% B to 56% B in 9 min; wave length: 220 nm; RT1(min): 9.67 the first peak was product) to afford 1-(4-(1,3-difluorocyclobutyl)pyridin-2-yl)-N-(6-methoxy-1-methyl-1H-indazol-7-yl)-1H-pyrazole-4-sulfonamide (I-377, 1.7 mg, 3.26%) as an off-white solid. LCMS (ES, m/z): [M+H]⁺=475; ¹H NMR: (400 MHz, DMSO-d₆) δ 2.86-2.97 (m, 2H), 3.04-3.13 (m, 2H), 3.34 (s, 3H), 4.26 (s, 3H), 5.44-5.59 (m, 1H), 6.85 (d, J=8.8 Hz, 1H), 7.59 (d, J=4.4 Hz, 2H), 7.95 (s, 1H), 7.99 (s, 2H), 8.59 (d, J=5.2 Hz, 1H), 8.70 (s, 1H), 9.78 (s, 1H).

### Example 382—Synthesis of 1-(4-(1,3-Difluorocyclobutyl) Pyridin-2-yl)-N-(6-Methoxy-1-Methyl-1H-Indazol-7-yl)-1H-Pyrazole-4-Sulfonamide (I-378)

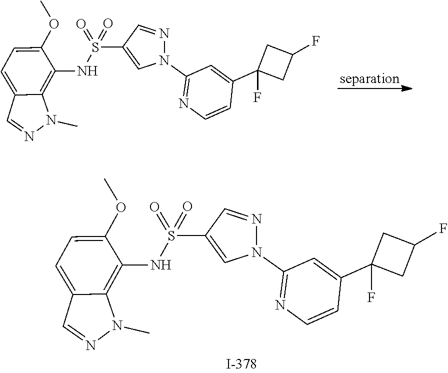 [SMILES: COc1ccc2cnn(C)c2c1NS(=O)(=O)c1cnn(-c2cc(C3(F)CC(F)C3)ccn2)c1 ; COc1ccc2cnn(C)c2c1NS(=O)(=O)c1cnn(-c2cc(C3(F)CC(F)C3)ccn2)c1]

1-(4-(1,3-difluorocyclobutyl)pyridin-2-yl)-N-(6-methoxy-1-methyl-1H-indazol-7-yl)-1H-pyrazole-4-sulfonamide(52 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 25 mL/min; gradient: 36% B to 56% B in 9 min; wave length: 220 nm; RT1(min): 9.67 the second peak was product) to afford 1-(4-(1,3-difluorocyclobutyl)pyridin-2-yl)-N-(6-methoxy-1-methyl-1H-indazol-7-yl)-1H-pyrazole-4-sulfonamide as a single steroeoisomer (I-378, 26.6 mg, 51.15%) as an off-white solid. LCMS (ES, m/z): [M+H]=475; ¹H NMR: (400 MHz, DMSO-d₆) δ 2.91-3.03 (m, 2H), 3.10-3.21 (m, 2H), 3.34 (s, 3H), 4.25 (s, 3H), 5.20-5.34 (m, 1H), 6.89 (d, J=8.8 Hz, 1H), 7.48 (s, 1H), 7.71 (d, J=8.8 Hz, 1H), 7.94 (d, J=1.6 Hz, 1H), 8.02 (s, 2H), 8.57 (d, J=5.2 Hz, 1H), 8.76 (s, 1H).

### Example 383—Synthesis of 1-((1-(4-(Trifluoromethyl)Pyridin-2-yl)-1H-Pyrazol-4-yl)Sulfonyl)-1,2,3,4-Tetrahydro-[1,4]Diazepino[3,2,1-HI]Indazole (I-379)

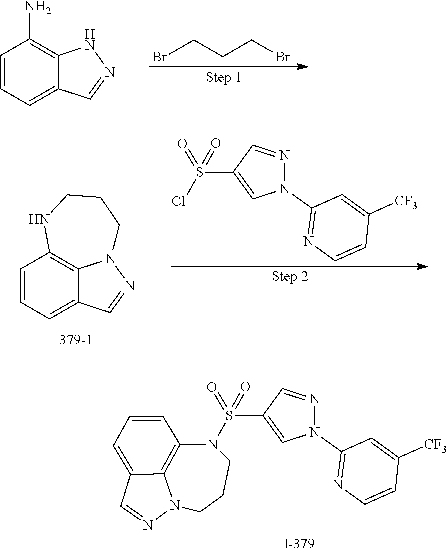 [SMILES: Nc1cccc2cn[nH]c12 ; BrCCCBr ; c1cc2c3c(c1)cnn3CCCN2 ; O=S(=O)(Cl)c1cnn(-c2cc(C(F)(F)F)ccn2)c1 ; O=S(=O)(c1cnn(-c2cc(C(F)(F)F)ccn2)c1)N1CCCn2ncc3cccc1c32]

Step 1: Synthesis of 379-1: A solution of 1H-indazol-7-amine (670 mg, 5.032 mmol, 1 equiv), 1,3-dibromopropane (1523.78 mg, 7.548 mmol, 1.5 equiv) and DIEA (1625.86 mg, 12.58 mmol, 2.5 equiv) in DMF (40 mL) was stirred overnight at 50° C. After the reaction was complete, the resulting mixture was diluted with water (400 mL). The resulting mixture was extracted with EtOAc (3×150 mL). The combined organic layers were washed with brine (400 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: ACN in water (10 mmol/L NH₄HCO₃), 0% to 100% gradient in 30 min; detector: UV 254 nm to afford 379-1 (100 mg, 11.47%) as a light brown solid.

Step 2: Synthesis of 1-((1-(4-(trifluoromethyl)pyridin-2-yl)-1H-pyrazol-4-yl)sulfonyl)-1,2,3,4-tetrahydro-[1,4]diazepino[3,2,1-hi]indazole (I-379): A solution of 379-1 (66 mg, 0.381 mmol, 1 equiv) and 1-[4-(trifluoromethyl)pyridin-2-yl]pyrazole-4-sulfonyl chloride (118.75 mg, 0.381 mmol, 1 equiv) in pyridine (4 mL) was stirred overnight at room temperature. After the reaction was complete, the resulting mixture was concentrated under vacuum. The residue was purified by reversed-phase flash chromatography with the following conditions: column: C₁₈ silica gel; mobile phase: ACN in water (10 mmol/L NH₄HCO₃), 0% to 100% gradient in 10 min; detector: UV 254 nm. The product was further purified by Prep-HPLC with the following conditions (column: Xselect CSH C₁₈ OBD Column 30*150 mm 5 μm; mobile phase A: water (0.1% FA), mobile phase B: ACN; flow rate: 60 mL/min; gradient: 47% B to 60% B in 9 min; wave length: 254 nm/220 nm; RT1(min): 8.7) to afford 1-((1-(4-(trifluoromethyl)pyridin-2-yl)-1H-pyrazol-4-yl)sulfonyl)-1,2,3,4-tetrahydro-[1,4]diazepino[3,2,1-hi]indazole (I-379, 5.0 mg, 2.93%) as an off-white solid. LCMS (ES, m/z): [M+1]⁺=449; ¹H NMR: (400 MHz, DMSO-d₆) δ 2.31 (t, J=5.2 Hz, 2H), 4.11 (t, J=5.2 Hz, 2H), 4.29 (t, J=6.0 Hz, 2H), 7.17 (t, J=8.0 Hz, 1H), 7.48 (d, J=7.6 Hz, 1H), 7.68 (d, J=8.0 Hz, 1H), 7.87 (d, J=4.4 Hz, 1H), 8.11 (d, J=9.6 Hz, 3H), 8.80 (d, J=4.8 Hz, 1H), 8.96 (s, 1H).

### Example 384—Synthesis of N-(6-Methoxy-1-Methyl-1H-Indazol-7-yl)-1-(4-(Pyrrolidin-1-yl)Pyridin-2-yl)-1H-Pyrazole-4-Sulfonamide (I-381)

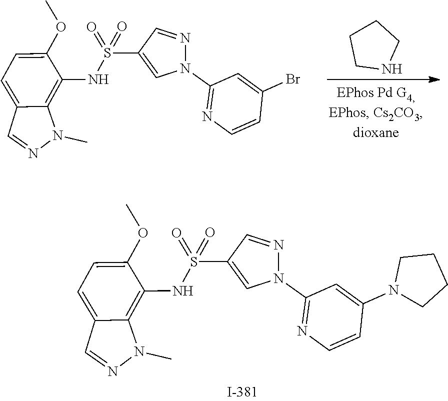 [SMILES: COc1ccc2cnn(C)c2c1NS(=O)(=O)c1cnn(-c2cc(Br)ccn2)c1 ; C1CCNC1 ; COc1ccc2cnn(C)c2c1NS(=O)(=O)c1cnn(-c2cc(N3CCCC3)ccn2)c1]

A mixture of 1-(4-bromopyridin-2-yl)-N-(6-methoxy-1-methylindazol-7-yl) pyrazole-4-sulfonamide (150 mg, 0.324 mmol, 1.00 equiv), pyrrolidine (46 mg, 0.648 mmol, 2.00 equiv), EPhos (17 mg, 0.032 mmol, 0.10 equiv), EPhos Pd G₄ (29 mg, 0.032 mmol, 0.10 equiv) and Cs₂CO₃ (316 mg, 0.972 mmol, 3.00 equiv) in dioxane (5 mL) was stirred for 2 h at 90° C. under a nitrogen atmosphere. The reaction was quenched with water at room temperature. The resulting mixture was extracted with EtOAc (3×5 mL). The combined organic layers were washed with brine (1×10 mL) and dried over anhydrous Na₂SO₄. After filtration, the filtrate was concentrated under reduced pressure. The crude product was purified by Prep-HPLC with the following conditions (column: RPPrep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 38% B to 58% B in 8 min; wave length: 220 nm; RT1(min): 8.38) to afford N-(6-methoxy-1-methylindazol-7-yl)-1-[4-(pyrrolidin-1-yl)pyridin-2-yl]pyrazole-4-sulfonamide (I-381, 10.2 mg, 6%) as an off-white solid. LCMS (ES, m/z): [M+H]⁺=454; ¹H NMR: ¹H NMR (400 MHz, chloroform-d) 2.06 (s, 4H), 3.45-3.47 (m, 7H), 4.41 (s, 3H), 6.35 (s, 1H), 6.44 (s, 1H), 6.66 (d, J=7.6 Hz, 1H), 6.92-7.03 (m, 1H), 7.50-7.69 (m, 2H), 7.96-7.99 (m, 2H), 8.73 (d, J=3.4 Hz, 1H).

### Example 385—Synthesis of 1-(4-(2-Cyanopropan-2-yl)Pyridin-2-yl)-N-(1-Methyl-1H-Indazol-7-yl)-1H-Pyrazole-4-Sulfonamide (I-382)

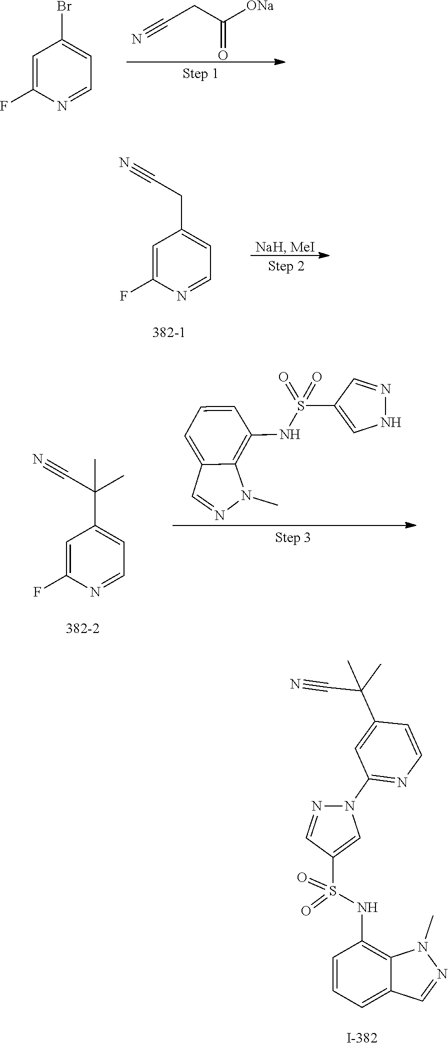

Step 1: Synthesis of 382-1: To a stirred mixture of 4-bromo-2-fluoropyridine (5 g, 28.411 mmol, 1 equiv) and sodium 2-cyanoacetate (6.08 g, 56.822 mmol, 2 equiv) in 1,3,5-trimethylbenzene (50 mL) were added bis(chloro(prop-2-en-1-yl)palladium) (1.04 g, 2.841 mmol, 0.1 equiv) and S-Phos (1.17 g, 2.841 mmol, 0.1 equiv) at room temperature under a nitrogen atmosphere. The resulting mixture was stirred for an additional 2 h at 140° C. The desired product could be detected by LCMS. The resulting mixture was filtered and the filter cake was washed with DCM (3×20 mL). The filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm to afford 2-(2-fluoropyridin-4-yl)acetonitrile (382-1, 500 mg, 12.93%) as a light brown solid.

Step 2: Synthesis of 382-2: To a stirred solution of 2-(2-fluoropyridin-4-yl)acetonitrile (300 mg, 2.204 mmol, 1 equiv) in THE (10 mL) were added NaH (264.43 mg, 6.612 mmol, 3 equiv, 60%) in portions at 0° C. under a nitrogen atmosphere. The resulting mixture was stirred for an additional 30 min at 0° C. To the above mixture was added CH₃I (938.41 mg, 6.612 mmol, 3 equiv) dropwise at 0° C. The resulting mixture was stirred for additional 5 h at room temperature. The desired product could be detected by LCMS. The reaction was quenched by the addition of sat. NH₄Cl (aq.) (10 mL) at 0° C. The resulting mixture was extracted with EtOAc (3×10 mL). The combined organic layers were washed with brine (1×10 mL) and dried over anhydrous Na₂SO₄. The organic phase was concentrated under reduced pressure to afford 2-(2-fluoropyridin-4-yl)-2-methylpropanenitrile (382-2, 300 mg, 82.91%) as a light brown solid. The crude product used in the next step without further purification.

Step 3: Synthesis of 1-(4-(2-cyanopropan-2-yl)pyridin-2-yl)-N-(1-methyl-1H-indazol-7-yl)-1H-pyrazole-4-sulfonamide (I-382): To a stirred mixture of N-(1-methylindazol-7-yl)-1H-pyrazole-4-sulfonamide (110 mg, 0.397 mmol, 1.00 equiv) and 2-(2-fluoropyridin-4-yl)-2-methylpropanenitrile (97.69 mg, 0.596 mmol, 1.5 equiv) in DMF (5 mL) was added Cs₂CO₃ (387.74 mg, 1.191 mmol, 3 equiv) at room temperature under a nitrogen atmosphere. The resulting mixture was stirred for an additional 4 h at 80° C. The desired product could be detected by LCMS. The reaction was quenched by the addition of sat. NH₄Cl (aq.) (5 mL) and water/ice (5 mL) at 0° C. The resulting mixture was extracted with CH₂Cl₂ (3×5 mL). The combined organic layers were washed with brine (1×10 mL) and dried over anhydrous Na₂SO₄. After filtration, the filtrate was concentrated under reduced pressure. The crude product (80 mg) was purified by Prep-HPLC with the following conditions (column: RP Prep OBD C18 column, 30*150 mm, 5 m; mobile phase A: water (10 mmol/L NH₄HCO₃+0.05% NH₃H₂O), mobile phase B: ACN; flow rate: 60 mL/min; gradient: 13% B to 38% B in 8 min; wave length: 220 nm; RT1(min): 9.15) to afford 1-[4-(1-cyano-1-methylethyl)pyridin-2-yl]-N-(1-methylindazol-7-yl)pyrazole-4-sulfonamide (I-382, 63 mg, 37.42%) as an off-white solid. LCMS (ES, m/z): [M+1]=422.10; H NMR (400 MHz, DMSO-d₆) δ 1.77 (s, 6H), 4.30 (s, 3H), 6.73 (d, J=7.4 Hz, 1H), 6.96 (t, J=7.8 Hz, 1H), 7.65 (d, J=6.6 Hz, 2H), 8.01-8.14 (m, 3H), 8.58 (d, J=5.4 Hz, 1H), 8.81 (s, 1H), 10.17 (brs, 1H).

### Example 386—Synthesis of 1-(4-(Azetidin-1-yl)Pyridin-2-yl)-N-(6-Methoxy-1-Methyl-1H-Pyrazolo[4,3-C]Pyridin-7-yl)-1H-Pyrazole-4-Sulfonamide (I-388)

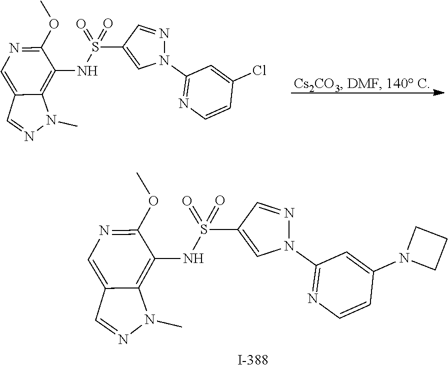 [SMILES: COc1ncc2cnn(C)c2c1NS(=O)(=O)c1cnn(-c2cc(Cl)ccn2)c1 ; COc1ncc2cnn(C)c2c1NS(=O)(=O)c1cnn(-c2cc(N3CCC3)ccn2)c1]

A solution of 1-(4-chloropyridin-2-yl)-N-{6-methoxy-1-methylpyrazolo[4,3-c]pyridin-7-yl}pyrazole-4-sulfonamide (80 mg, 0.191 mmol, 1 equiv), Cs₂CO₃ (186.25 mg, 0.573 mmol, 3 equiv) and azetidine (21.76 mg, 0.382 mmol, 2 equiv) in DMF (3 mL) was stirred overnight at 140° C. under a nitrogen atmosphere. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (70 mg) was purified by Prep-HPLC with the following conditions (column: XBridge Shield RP18 OBD column, 30*150 mm, 5 μm; mobile phase A: water (10 mmol/L NH₄HCO₃+0.05% NH₃H₂O), mobile phase B: ACN; flow rate: 60 mL/min; gradient: 23% B to 45% B in 7 min; wave length: 254 nm/220 nm; RT1(min): 6.87; number of runs: 2) to afford 1-[4-(azetidin-1-yl)pyridin-2-yl]-N-{6-methoxy-1-methylpyrazolo[4,3-c]pyridin-7-yl}pyrazole-4-sulfonamide (I-388, 26.1 mg, 30.97%) as an off-white solid. LCMS (ES, m/z): [M+H]=441; ¹H NMR (400 MHz, DMSO-d₆) δ 2.36-2.46 (m, 2H), 3.42 (s, 3H), 4.02 (t, J=7.2 Hz, 4H), 4.21 (s, 3H), 6.36 (dd, J=2.2, 5.8 Hz, 1H), 6.80 (d, J=2.2 Hz, 1H), 7.92 (d, J=0.8 Hz, 1H), 8.02 (d, J=5.7 Hz, 1H), 8.22 (s, 1H), 8.65 (s, 1H), 8.66 (s, 1H), 9.85 (s, 1H).

### Example 387—Synthesis of 1-(4-(3-Azabicyclo[3.1.1]Heptan-3-yl)Pyridin-2-yl)-N-(1-Methyl-1H-Indazol-7-yl)-1H-Pyrazole-4-Sulfonamide (I-389)

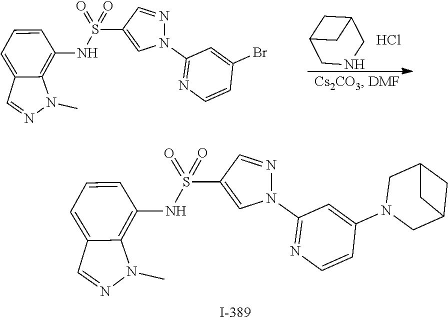 [SMILES: Cn1ncc2cccc(NS(=O)(=O)c3cnn(-c4cc(Br)ccn4)c3)c21 ; C1NCC2CC1C2 ; Cl ; Cn1ncc2cccc(NS(=O)(=O)c3cnn(-c4cc(N5CC6CC(C6)C5)ccn4)c3)c21]

To a stirred solution of 1-(4-chloropyridin-2-yl)-N-(1-methylindazol-7-yl) pyrazole-4-sulfonamide (60 mg, 0.154 mmol, 1 equiv)) and 3-azabicyclo [3.1.1] heptane hydrochloride (103.09 mg, 0.770 mmol, 5 equiv) in DMF (2 mL) were added Cs₂CO₃ (150.83 mg, 0.462 mmol, 3 equiv) in portions at room temperature under a nitrogen atmosphere. The resulting mixture was stirred for 8 h at 100° C. under a nitrogen atmosphere. The resulting mixture was diluted with water (10 mL). The resulting mixture was extracted with EtOAc (3×10 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 1-(4-{3-azabicyclo [3.1.1] heptan-3-yl} pyridin-2-yl)-N-(1-methylindazol-7-yl) pyrazole-4-sulfonamide (I-389, 17.4 mg, 24.68%) as an off white solid. LCMS (ES, m/z): [M+H]⁺=450; ¹H NMR: (400 MHz, DMSO-d₆) δ 1.48 (dd, J=7.0, 2.8 Hz, 2H), 2.31 (d, J=8.0 Hz, 2H), 2.67 (d, J=6.4 Hz, 2H), 3.64 (s, 4H), 4.37 (s, 3H), 6.69 (dd, J=6.0, 2.4 Hz, 1H), 6.74 (dd, J=8.0, 1.0 Hz, 1H), 6.95-7.01 (m, 1H), 7.25 (d, J=2.4 Hz, 1H), 7.67-7.72 (m, 1H), 7.80 (s, 1H), 8.02 (t, J=3.0 Hz, 2H), 8.70 (s, 1H).

### Example 388—Synthesis of 1-(4-(Azepan-1-yl)Pyridin-2-yl)-N-(1-Methyl-1H-Indazol-7-yl)-1H-Pyrazole-4-Sulfonamide (I-391)

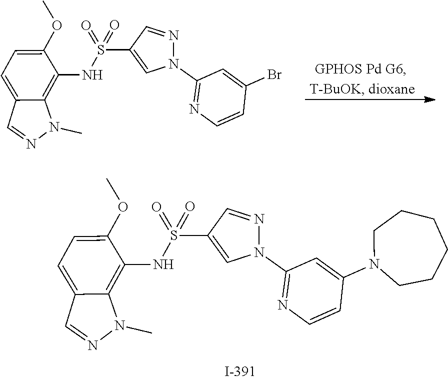 [SMILES: COc1ccc2cnn(C)c2c1NS(=O)(=O)c1cnn(-c2cc(Br)ccn2)c1 ; COc1ccc2cnn(C)c2c1NS(=O)(=O)c1cnn(-c2cc(N3CCCCCC3)ccn2)c1]

To a stirred solution of 1-(4-bromopyridin-2-yl)-N-(1-methylindazol-7-yl)pyrazole-4-sulfonamide (80 mg, 0.185 mmol, 1 equiv) and hexamethyleneimine (91.56 mg, 0.925 mmol, 5 equiv) in dioxane (3 mL) were added {2-[2,6-bis(propan-2-yl)phenyl]-6-(tert-butoxy)-3-methoxyphenyl}dicyclohexylphosphane (9.91 mg, 0.018 mmol, 0.1 equiv), GPhos Pd G6 TES (17.46 mg, 0.018 mmol, 0.1 equiv) and t-BuOK (62.16 mg, 0.555 mmol, 3 equiv). The resulting mixture was stirred overnight at 100° C. under a nitrogen atmosphere. The reaction was quenched with water at room temperature. The resulting mixture was extracted with EtOAc (2×50 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 35% to 50% gradient in 10 min; detector: UV 254 nm. This resulted in 1-[4-(azepan-1-yl)pyridin-2-yl]-N-(1-methylindazol-7-yl)pyrazole-4-sulfonamide (I-391, 15 mg, 17.96%) as an off-white solid. LCMS (ES, m/z): [M+H]⁺=452; ¹H NMR: (400 MHz, DMSO-d₆) δ 1.43-1.51 (m, 4H), 1.73 (m, 4H), 3.54 (t, J=6.0 Hz, 4H), 4.33 (s, 3H), 6.66 (m, J=6.0, 2.6 Hz, 1H), 6.75 (d, J=7.8 Hz, 1H), 6.87 (t, J=7.8 Hz, 1H), 7.09 (d, J=2.4 Hz, 1H), 7.35 (s, 1H), 7.87 (s, 1H), 7.91-8.01 (m, 2H), 8.62 (s, 1H).

### Example 389—Synthesis of 9-((1-(4-(Trifluoromethyl)Pyridin-2-yl)-1H-Pyrazol-4-yl)Sulfonyl)-6,7,8,9-Tetrahydro-1H-Pyrazolo[4,3-H]Quinoline (I-393) 1-393

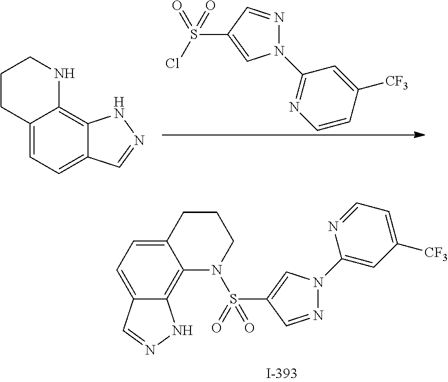 [SMILES: c1cc2cn[nH]c2c2c1CCCN2 ; O=S(=O)(Cl)c1cnn(-c2cc(C(F)(F)F)ccn2)c1 ; O=S(=O)(c1cnn(-c2cc(C(F)(F)F)ccn2)c1)N1CCCc2ccc3cn[nH]c3c21]

A solution of 1H,6H,7H,8H,9H,10H-azepino[3,2-g]indazole (25 mg, 0.134 mmol, 1 equiv) and 1-[4-(trifluoromethyl)pyridin-2-yl]pyrazole-4-sulfonyl chloride (49.93 mg, 0.161 mmol, 1.2 equiv) in pyridine (3 mL) was stirred overnight at room temperature. After the reaction was complete, the resulting mixture was concentrated under vacuum to give the crude. The crude product was purified by Prep-HPLC with the following conditions (column: Xselect CSH C₁₈ OBD column 30*150 mm 5 μm; mobile phase A: water (0.1% FA), mobile phase B: ACN; flow rate: 60 mL/min; gradient: 47% B to 58% B in 9 min; wave length: 254 nm/220 nm nm; RT₁(min): 8.5, 9(min)) to afford 9-((1-(4-(trifluoromethyl)pyridin-2-yl)-1H-pyrazol-4-yl)sulfonyl)-6,7,8,9-tetrahydro-1H-pyrazolo[4,3-h]quinoline (I-393, 7.3 mg, 11.42%) as a light yellow solid. LCMS (ES, m/z): [M+1]⁺=449; ¹H NMR: (400 MHz, DMSO-d₆) δ 1.86 (t, J=6.8 Hz, 2H), 2.43 (t, J=7.2 Hz, 2H), 3.76 (t, J=6.4 Hz, 2H), 6.88 (d, J=8.0 Hz, 1H), 7.62 (d, J=8.4 Hz, 1H), 7.88 (d, J=2.4 Hz, 1H), 8.04 (s, 2H), 8.19 (s, 1H), 8.81 (d, J=5.2 Hz, 1H), 9.01 (s, 1H), 12.83 (s, 1H).

### Example 390—Synthesis of 1-((1-(4-(Trifluoromethyl)Pyridin-2-yl)-1H-Pyrazol-4-yl)Sulfonyl)-2,3,4,5-Tetrahydro-1H-[1,4]Diazocino[3,2,1-HI]Indazole (I-396)

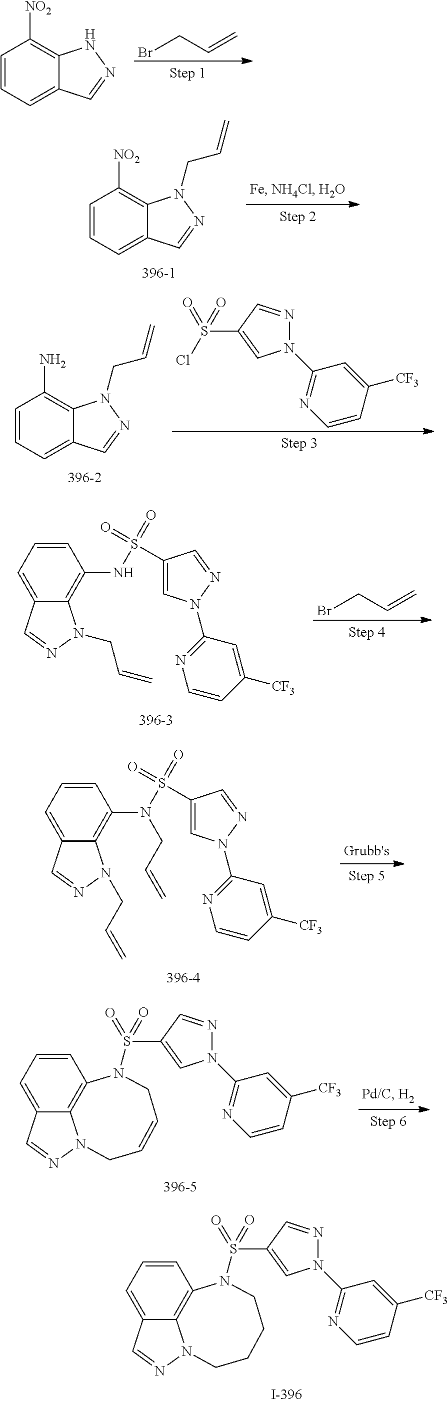

Step 1: Synthesis of 396-1: To a stirred mixture of 7-nitroindazole (2 g, 12.2 mmol, 1.00 equiv) in DMF (50 mL) was added NaH (0.740 g, 30.6 mmol, 2.50 equiv) dropwise at 0° C. under a nitrogen atmosphere. The resulting mixture was stirred for 1 h at room temperature under a nitrogen atmosphere. To the above mixture was added allyl bromide (2.22 g, 18.3 mmol, 1.50 equiv) dropwise at 0° C. The resulting mixture was stirred for an additional 3 h at room temperature. The reaction was quenched with water at room temperature. The resulting mixture was extracted with EtOAc (3×30 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 20% to 40% gradient in 10 min; detector: UV 254 nm. This resulted in 7-nitro-1-(prop-2-en-1-yl)indazole (396-1, 1.8 g, 72%) as a yellow oil. LCMS (ES, m/z): [M+H]⁺=204.

Step 2: Synthesis of 396-2: A mixture of 7-nitro-1-(prop-2-en-1-yl) indazole (1.8 g, 8.85 mmol, 1.00 equiv), Fe (2.47 g, 44.2 mmol, 5.00 equiv) and NH₄Cl (2.37 g, 44.2 mmol, 5.00 equiv) in EtOH (50 mL) and H₂O (10 mL) was stirred overnight at 60° C. under a nitrogen atmosphere. The resulting mixture was filtered and the filter cake was washed with EtOAc (3×50 mL). The filtrate was concentrated under reduced pressure. The residue was purified by reversed-phase flash chromatography with the following conditions: column: C18 silica gel; mobile phase: MeCN in water (10 mmol/L NH₄HCO₃), 10% to 20% gradient in 10 min; detector: UV 254 nm. This resulted in 1-(prop-2-en-1-yl)indazol-7-amine (396-2, 1.5 g, 97%) as a yellow oil. LCMS (ES, m/z): [M+H]⁺=174.

Step 3: Synthesis of 396-3: A mixture of 1-(prop-2-en-1-yl) indazol-7-amine (300 mg, 1.73 mmol, 1.00 equiv) and 1-[4-(trifluoromethyl) pyridin-2-yl] pyrazole-4-sulfonyl chloride (647 mg, 2.07 mmol, 1.20 equiv) in pyridine (10 mL) was stirred overnight at room temperature under a nitrogen atmosphere. The reaction was quenched with water at room temperature. The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₃·H₂O), 30% to 50% gradient in 10 min; detector: UV 254 nm. This resulted in N-[1-(prop-2-en-1-yl)indazol-7-yl]-1-[4-(trifluoromethyl)pyridin-2-yl]pyrazole-4-sulfonamide (396-3, 600 mg, 77%) as a yellow solid. LCMS (ES, m/z): [M+H]⁺=449.

Step 4: Synthesis of 396-4: A mixture of N-[1-(prop-2-en-1-yl) indazol-7-yl]-1-[4-(trifluoromethyl) pyridin-2-yl] pyrazole-4-sulfonamide (500 mg, 1.11 mmol, 1.00 equiv), allyl bromide (674 mg, 5.57 mmol, 5.00 equiv), K₂CO₃ (462 mg, 3.34 mmol, 3.00 equiv) and KI (18 mg, 0.112 mmol, 0.10 equiv) in DMF (5 mL) was stirred for 2 h at room temperature under a nitrogen atmosphere. The reaction was quenched with water at room temperature. The resulting mixture was extracted with EtOAc (3×5 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 60% gradient in 10 min; detector: UV 254 nm. This resulted in N-(prop-2-en-1-yl)-N-[1-(prop-2-en-1-yl)indazol-7-yl]-1-[4-(trifluoromethyl)pyridin-2-yl]pyrazole-4-sulfonamide (396-4, 300 mg, 55%) as a yellow solid. LCMS (ES, m/z): [M+H]⁺=489.

Step 5: Synthesis of 396-5: A mixture of N-(prop-2-en-1-yl)-N-[1-(prop-2-en-1-yl) indazol-7-yl]-1-[4-(trifluoromethyl) pyridin-2-yl] pyrazole-4-sulfonamide (300 mg, 0.614 mmol, 1.00 equiv) and Grubbs 2^{nd} generation catalyst (104 mg, 0.123 mmol, 0.20 equiv) in THF (5 mL) was stirred for 2 h at 50° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 50% to 70% gradient in 10 min; detector: UV 254 nm. This resulted in (11Z)-9-{1-[4-(trifluoromethyl)pyridin-2-yl]pyrazol-4-ylsulfonyl}-1,2,9-triazatricyclo[6.5.1.0\hat{ }{4,14}]tetradeca-2,4,6,8(14),11-pentaene (396-5, 50 mg, 17%) as a yellow solid. LCMS (ES, m/z): [M+H]⁺=461.

Step 6: Synthesis of 1-((1-(4-(trifluoromethyl)pyridin-2-yl)-1H-pyrazol-4-yl)sulfonyl)-2,3,4,5-tetrahydro-1H-[1,4]diazocino[3,2,1-hi]indazole (I-396): A mixture of (11Z)-9-{1-[4-(trifluoromethyl) pyridin-2-yl] pyrazol-4-ylsulfonyl}-1,2,9-triazatricyclo [6.5.1.0\hat{ } {4,14}] tetradeca-2,4,6,8(14),11-pentaene (50 mg, 0.109 mmol, 1.00 equiv) and Pd/C (5.78 mg, 0.054 mmol, 0.5 equiv) in MeOH (3 mL) was stirred overnight at room temperature under a hydrogen atmosphere. The resulting mixture was filtered and the filter cake was washed with MeOH (3×5 mL). The filtrate was concentrated under reduced pressure. The residue was purified by reversed-phase flash chromatography with the following conditions: column: C18 silica gel; mobile phase: MeCN in water (10 mmol/L NH₄HCO₃), 60% to 80% gradient in 10 min; detector: UV 254 nm. This resulted in 9-{1-[4-(trifluoromethyl)pyridin-2-yl]pyrazol-4-ylsulfonyl}-1,2,9-triazatricyclo[6.5.1.0{4,14}]tetradeca-2,4,6,8(14)-tetraene (I-396, 23.9 mg, 47%) as a white solid. LCMS (ES, m/z): [M+H]⁺=463; ¹H NMR (400 MHz, DMSO-d₆) δ 1.13 (s, 1H), 1.78 (s, 1H), 1.91 (s, 1H), 2.06 (s, 1H), 3.31 (s, 1H), 4.33 (s, 1H), 4.66 (s, 1H), 4.85 (s, 1H), 7.08-7.17 (m, 2H), 7.79 (dd, J=6.5, 2.6 Hz, 1H), 7.91 (d, J=5.2 Hz, 1H), 8.11 (s, 1H), 8.25 (s, 1H), 8.45 (d, J=2.2 Hz, 1H), 8.87 (d, J=5.2 Hz, 1H), 9.23 (s, 1H).

### Example 391—Synthesis of N-(6-Methoxy-1-Methyl-1H-Pyrazolo[4,3-C]Pyridin-7-yl)-1-(4-(Pyrrolidin-1-yl)Pyridin-2-yl)-1H-Pyrazole-4-Sulfonamide (I-398)

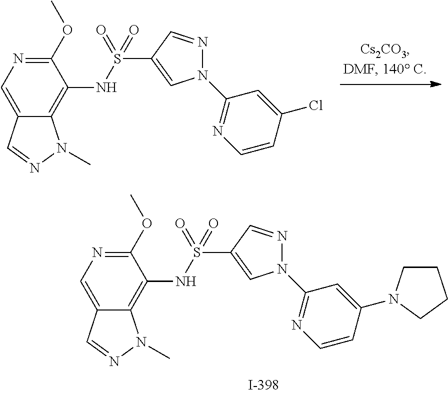 [SMILES: COc1ncc2cnn(C)c2c1NS(=O)(=O)c1cnn(-c2cc(Cl)ccn2)c1 ; COc1ncc2cnn(C)c2c1NS(=O)(=O)c1cnn(-c2cc(N3CCCC3)ccn2)c1]

A solution of 1-(4-chloropyridin-2-yl)-N-{6-methoxy-1-methylpyrazolo[4,3-c]pyridin-7-yl}pyrazole-4-sulfonamide (80 mg, 0.191 mmol, 1 equiv), Cs₂CO₃ (186.25 mg, 0.573 mmol, 3 equiv), and pyrrolidine (27.10 mg, 0.382 mmol, 2 equiv) in DMF (3 mL) was stirred overnight at 140° C. under a nitrogen atmosphere. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (50 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 21% B to 46% B in 8 min; wave length: 220 nm; RT1(min): 7.56) to afford N-{6-methoxy-1-methylpyrazolo[4,3-c]pyridin-7-yl}-1-[4-(pyrrolidin-1-yl)pyridin-2-yl]pyrazole-4-sulfonamide (I-398, 19 mg, 21.85%) as an off-white solid. LCMS (ES, m/z): [M+H]=455; ¹H NMR (400 MHz, DMSO-d₆) 1.99 (q, J=3.6 Hz, 4H), 3.35 (q, J=3.6 Hz, 4H), 3.42 (s, 3H), 4.22 (s, 3H), 6.54 (dd, J=2.4, 6.0 Hz, 1H), 6.96 (d, J=2.4 Hz, 1H), 7.92 (s, 1H), 8.09 (d, J=6.0 Hz, 1H), 8.23 (d, J=1.4 Hz, 1H), 8.66 (s, 1H), 8.67 (s, 1H).

### Example 392—Synthesis of 1-(4-(2-Azabicyclo[2.1.1]Hexan-2-yl)Pyridin-2-yl)-N-(1-Methyl-1H-Indazol-7-yl)-1H-Pyrazole-4-Sulfonamide (I-409)

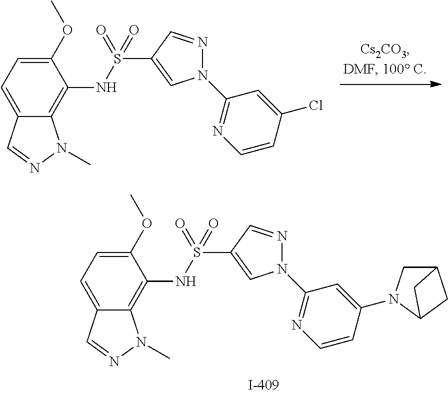 [SMILES: COc1ccc2cnn(C)c2c1NS(=O)(=O)c1cnn(-c2cc(Cl)ccn2)c1 ; COc1ccc2cnn(C)c2c1NS(=O)(=O)c1cnn(-c2cc(N3CC4CC3C4)ccn2)c1]

To a stirred solution of 1-(4-chloropyridin-2-yl)-N-(1-methylindazol-7-yl)pyrazole-4-sulfonamide (100 mg, 0.257 mmol, 1 equiv) and 2-azabicyclo[2.1.1]hexane (106.90 mg, 1.285 mmol, 5 equiv) in DMF (2 mL) was added Cs₂CO₃ (335.18 mg, 1.028 mmol, 4 equiv). The resulting mixture was stirred overnight at 100° C. under a nitrogen atmosphere. The reaction was quenched with water at room temperature. The resulting mixture was extracted with EtOAc (2×50 mL). The combined organic layers were washed with brine (1×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 50% gradient in 10 min; detector: UV 254 nm. This resulted in 1-(4-{2-azabicyclo[2.1.1]hexan-2-yl}pyridin-2-yl)-N-(1-methylindazol-7-yl)pyrazole-4-sulfonamide (I-409, 18.8 mg, 16.08%) as a light pink solid. LCMS (ES, m/z): [M+H]⁺=436; ¹H NMR: (400 MHz, DMSO-d₆) δ 1.37 (m, J=4.6, 1.8 Hz, 2H), 2.03 (m, 2H), 2.95-3.02 (m, 1H), 3.37 (m, 2H), 4.28 (s, 3H), 4.69 (d, J=6.8 Hz, 1H), 6.68 (d, J=7.4 Hz, 2H), 6.98 (t, J=8.0 Hz, 1H), 7.09 (s, 1H), 7.70 (d, J=8.0 Hz, 1H), 7.94 (s, 1H), 7.98 (d, J=5.8 Hz, 1H), 8.09 (s, 1H), 8.72 (s, 1H), 9.97 (s, 1H).

### Example 393—Synthesis of 1-(4-(1,1-Difluoroethyl)Pyridin-2-yl)-N-(6-Methoxy-1-(Methyl-D3)-1H-Pyrazolo[4,3-C]Pyridin-7-yl)-1H-Pyrazole-4-Sulfonamide (I-411)

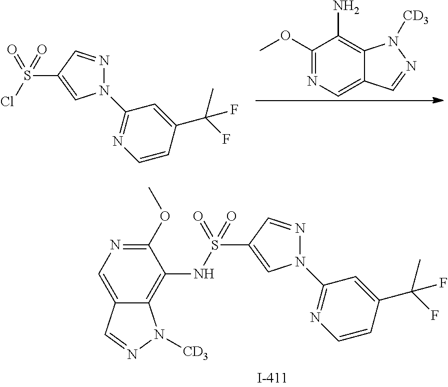

A solution of 1-[4-(1,1-difluoroethyl)pyridin-2-yl]pyrazole-4-sulfonyl chloride (150 mg, 0.487 mmol, 1 equiv) and 6-methoxy-1-(²H₃)methylpyrazolo[4,3-c]pyridin-7-amine (88.34 mg, 0.487 mmol, 1 equiv) in pyridine (1 mL) was stirred for 16 h at 140° C. under an air atmosphere. The resulting mixture was diluted with water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 1-[4-(1,1-difluoroethyl)pyridin-2-yl]-N-[6-methoxy-1-(²H₃)methylpyrazolo[4,3-c]pyridin-7-yl]pyrazole-4-sulfonamide (I-411, 22.7 mg, 10.24%) as a white solid. LCMS (ES, m/z): [M+1]=453; ¹H NMR: (400 MHz, DMSO-d₆) δ 2.06 (t, J=19.2 Hz, 3H), 3.44 (s, 3H), 7.67 (dd, J=1.6, 5.2 Hz, 1H), 8.05 (s, 1H), 8.06 (s, 1H), 8.22 (s, 1H), 8.66 (s, 1H), 8.72 (d, J=4.8 Hz, 1H), 8.79 (s, 1H), 9.95 (s, 1H).

### Example 394—Synthesis of 1-(4-(1,1-Difluoroethyl)Pyridin-2-yl)-N-(6-Methoxy-1-(Methyl-D3)-1H-Indazol-7-yl)-1H-Pyrazole-4-Sulfonamide (I-412)

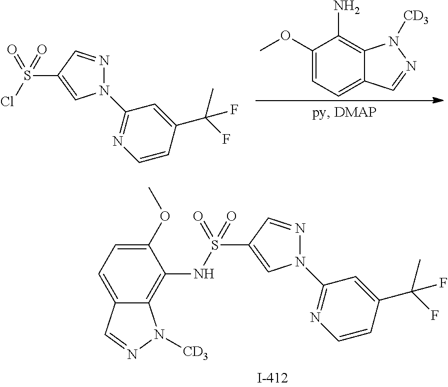

To a stirred solution of 1-[4-(1,1-difluoroethyl) pyridin-2-yl] pyrazole-4-sulfonyl chloride (70 mg, 0.227 mmol, 1 equiv) and 6-methoxy-1-(2H3) methylindazol-7-amine (49.20 mg, 0.272 mmol, 1.2 equiv) in pyridine (2 mL) was added DMAP (2.78 mg, 0.023 mmol, 0.1 equiv) in portions at room temperature under a nitrogen atmosphere. The resulting mixture was stirred for 2 h at room temperature under a nitrogen atmosphere. The resulting mixture was diluted with water (10 mL). The resulting mixture was extracted with EtOAc (3×20 mL). The combined organic layers were washed with brine (1×10 mL) and dried over anhydrous Na₂SO₄. The resulting mixture was concentrated under reduced pressure. The residue was purified by reversed-phase flash chromatography with the following conditions: column: C18 silica gel; mobile phase: MeCN in water (10 mmol/L NH₄HCO₃), 40% to 50% gradient in 10 min; detector: UV 254 nm. The resulting mixture was concentrated under reduced pressure. This resulted in 1-[4-(1,1-difluoroethyl) pyridin-2-yl]-N-[6-methoxy-1-(2H3) methylindazol-7-yl] pyrazole-4-sulfonamide (I-412, 58.1 mg, 55.83%) as an off-white solid. LCMS (ES, m/z): [M+H]⁺=452; ¹H NMR: (400 MHz, DMSO-d₆) δ 2.06 (t, J=19.4 Hz, 3H), 3.31 (s, 3H), 6.88 (d, J=8.0 Hz, 1H), 7.65-7.70 (m, 2H), 7.98 (s, 1H), 8.03-8.08 (m, 2H), 8.69 (d, J=4.0 Hz, 1H), 8.75 (d, J=4.0 Hz, 1H), 9.82 (s, 1H).

### Example 395—Synthesis of 1-(4-(1-Fluoro-3-Methylcyclobutyl) Pyridin-2-yl)-N-(6-Methoxy-1-Methyl-1H-Indazol-7-yl)-1H-Pyrazole-4-Sulfonamide (I-413)

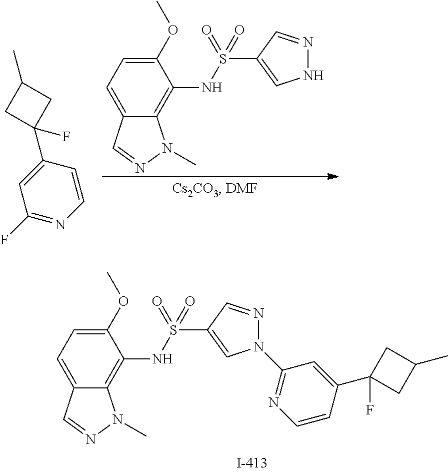

A solution of 2-fluoro-4-(1-fluoro-3-methylcyclobutyl)pyridine (80 mg, 0.437 mmol, 1 equiv), N-(6-methoxy-1-methylindazol-7-yl)-1H-pyrazole-4-sulfonamide (201 mg, 0.654 mmol, 1.50 equiv) and Cs₂CO₃ (426.83 mg, 1.311 mmol, 3 equiv) in DMF (7 mL) was stirred overnight at 80° C. After the reaction was complete, the mixture was purified by reversed-phase flash chromatography with the following conditions: column: C₁₈ silica gel; mobile phase: ACN in water (10 mmol/L NH₄HCO₃), 0% to 100% gradient in 30 min; detector: UV 254 nm to afford 1-(4-(1-fluoro-3-methylcyclobutyl)pyridin-2-yl)-N-(6-methoxy-1-methyl-1H-indazol-7-yl)-1H-pyrazole-4-sulfonamide (I-413, 55.2 mg, 26.76%) as an off-white solid. LCMS (ES, m/z): [M+1]⁺=471; ¹H NMR: (400 MHz, DMSO-d₆) δ 1.18-1.24 (m, 3H), 2.21-2.35 (m, 2H), 2.63-2.80 (m, 3H), 3.33 (d, J=4.0 Hz, 3H), 4.25 (s, 3H), 6.87 (d, J=8.8 Hz, 1H), 7.56 (t, J=5.2 Hz, 1H), 7.66 (d, J=8.8 Hz, 1H), 7.94-8.01 (m, 3H), 8.56 (t, J=4.4 Hz, 1H), 8.73 (s, 1H), 9.78 (s, 1H).

### Example 396—Synthesis of 1-(4-(3,3-Difluoropyrrolidin-1-yl)Pyridin-2-yl)-N-(6-Methoxy-1-Methyl-1H-Pyrazolo[4,3-C]Pyridin-7-yl)-1H-Pyrazole-4-Sulfonamide (I-428)

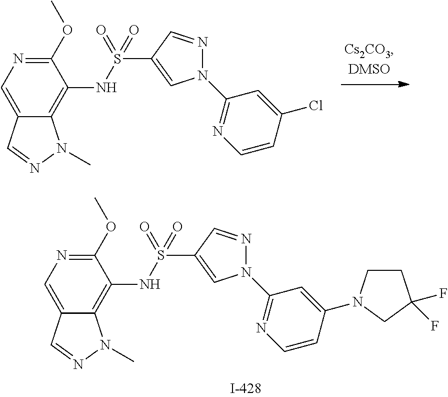

A solution of 1-(4-chloropyridin-2-yl)-N-{6-methoxy-1-methylpyrazolo[4,3-c]pyridin-7-yl}pyrazole-4-sulfonamide (100 mg, 0.238 mmol, 1 equiv), Cs₂CO₃ (232.82 mg, 0.714 mmol, 3 equiv) and 3,3-difluoropyrrolidine hydrochloride (68.39 mg, 0.476 mmol, 2 equiv) in DMSO (3 mL) was stirred overnight at 140° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product (40 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 21% B to 46% B in 8 min; wave length: 220 nm; RT1(min): 7.2) to afford 1-(4-(3,3-difluoropyrrolidin-1-yl)pyridin-2-yl)-N-(6-methoxy-1-methyl-1H-pyrazolo[4,3-c]pyridin-7-yl)-1H-pyrazole-4-sulfonamide (I-428, 8.9 mg, 7.60%) as a white solid. LCMS (ES, m/z): [M+H]⁺=491; ¹H NMR: (400 MHz, DMSO-d₆) δ 2.58-2.63 (m, 2H). 3.34 (s, 3H), 3.69 (t, J=7.2 Hz, 2H), 3.82 (t, J=12.8 Hz, 2H), 4.31 (s, 3H), 6.57 (dd, J=2.4, 5.6 Hz, 1H), 7.12 (d, J=2.4 Hz, 1H), 7.81 (d, J=0.8 Hz, 1H), 8.05 (d, J=5.6 Hz, 1H), 8.11 (s, 1H), 8.57 (s, 1H), 8.67 (s, 1H).

### Example 397—Synthesis of 1-(4-(2-Azabicyclo[2.1.1]Hexan-2-yl)Pyridin-2-yl)-N-(6-Methoxy-1-Methyl-1H-Indazol-7-yl)-1H-Pyrazole-4-Sulfonamide (I-432)

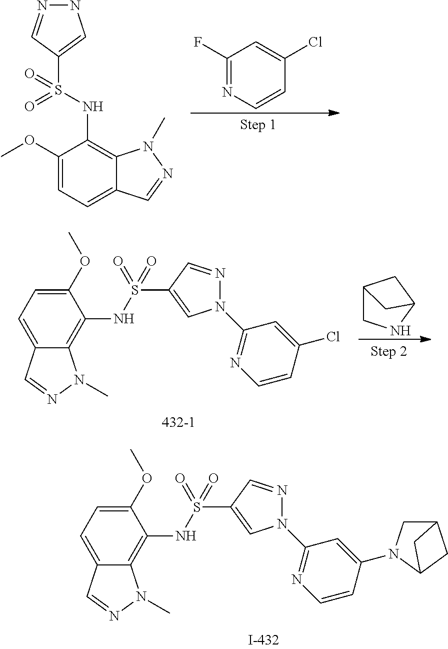

Step 1: Synthesis of 432-1: A solution of N-(6-methoxy-1-methylindazol-7-yl)-1H-pyrazole-4-sulfonamide (1 g, 3.254 mmol, 1 equiv) and 4-chloro-2-fluoropyridine (641.96 mg, 4.881 mmol, 1.5 equiv) and Cs₂CO₃ (3.18 g, 9.762 mmol, 3 equiv) in DMF (10 mL) was stirred for 2 h at 80° C. under a nitrogen atmosphere. The resulting mixture was diluted with water (40 mL). The resulting mixture was extracted with EtOAc (3×100 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 70% gradient in 10 min; detector: UV 254 nm. This resulted in 1-(4-chloropyridin-2-yl)-N-(6-methoxy-1-methylindazol-7-yl)pyrazole-4-sulfonamide (432-1, 600 mg, 44.02%) as a brown yellow solid.

Step 2: Synthesis of 1-(4-(2-azabicyclo[2.1.1]hexan-2-yl)pyridin-2-yl)-N-(6-methoxy-1-methyl-1H-indazol-7-yl)-1H-pyrazole-4-sulfonamide (I-432): A solution of 1-(4-chloropyridin-2-yl)-N-(6-methoxy-1-methylindazol-7-yl)pyrazole-4-sulfonamide (100 mg, 0.239 mmol, 1 equiv) and 2-azabicyclo[2.1.1]hexane (100.0 mg, 1.203 mmol, 5.04 equiv) and Cs₂CO₃ (250 mg, 0.767 mmol, 3.21 equiv) in DMF (2 mL) was stirred overnight at 100° C. under a nitrogen atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The crude product (100 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 31% B to 56% B in 8 min; wave length: 220 nm; RT1(min): 7.23) to afford 1-(4-{2-azabicyclo[2.1.1]hexan-2-yl}pyridin-2-yl)-N-(6-methoxy-1-methylindazol-7-yl)pyrazole-4-sulfonamide (I-432, 21.7 mg, 19.52%) as an off-white solid. LCMS (ES, m/z): [M+1]=466; ¹H NMR: 1H NMR (400 MHz, chloroform-d) δ 1.46 (dd, J=4.6, 2.0 Hz, 2H), 1.98-2.10 (m, 2H), 3.03 (dt, J=6.6, 3.2 Hz, 1H), 3.38-3.42 (m, 5H), 4.40 (s, 3H), 4.48-4.54 (m, 1H), 6.39-6.50 (m, 2H), 6.66 (d, J=8.8 Hz, 1H), 7.07 (d, J=2.2 Hz, 1H), 7.56-7.65 (m, 2H), 7.92 (s, 1H), 7.97 (d, J=5.8 Hz, 1H), 8.73 (s, 1H).

### Example 398—Synthesis of 1-(4-(Dimethylamino)Pyridin-2-yl)-N-(1-Methyl-1H-Indazol-7-yl)-1H-Pyrazole-4-Sulfonamide (I-437)

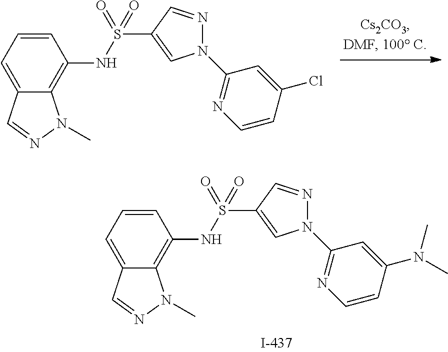 [SMILES: Cn1ncc2cccc(NS(=O)(=O)c3cnn(-c4cc(Cl)ccn4)c3)c21 ; CN(C)c1ccnc(-n2cc(S(=O)(=O)Nc3cccc4cnn(C)c34)cn2)c1]

A solution of 1-(4-chloropyridin-2-yl)-N-(1-methylindazol-7-yl)pyrazole-4-sulfonamide (100 mg, 0.257 mmol, 1 equiv), 3,3-difluoropyrrolidine (137.73 mg, 1.285 mmol, 5 equiv) and Cs₂CO₃ (251.38 mg, 0.771 mmol, 3 equiv) in DMF (2 mL) was stirred overnight at 100° C. under a nitrogen atmosphere. The reaction was quenched with water at room temperature. The resulting mixture was extracted with EtOAc (2×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 1-[4-(dimethylamino)pyridin-2-yl]-N-(1-methylindazol-7-yl)pyrazole-4-sulfonamide (I-437, 18.9 mg, 36.98%) as an off-white solid. LCMS (ES, m/z): [M+H]⁺=398; ¹H NMR: (400 MHz, DMSO-d₆): δ 3.06 (s, 6H), 4.28 (s, 3H), 6.65-6.72 (m, 2H), 6.98 (t, J=7.8 Hz, 1H), 7.12 (d, J=2.4 Hz, 1H), 7.70 (d, J=8.0 Hz, 1H), 7.94 (s, 1H), 8.03 (d, J=6.0 Hz, 1H), 8.08 (s, 1H), 8.72 (s, 1H), 9.97 (s, 1H).

### Example 399—Synthesis of 1-(7,8-Dihydro-4H,6H-Pyrazolo[5,1-C][1,4]Oxazepin-3-yl)-N-(1-Methyl-1H-Indazol-7-yl)-1H-Pyrazole-4-Sulfonamide (I-447)

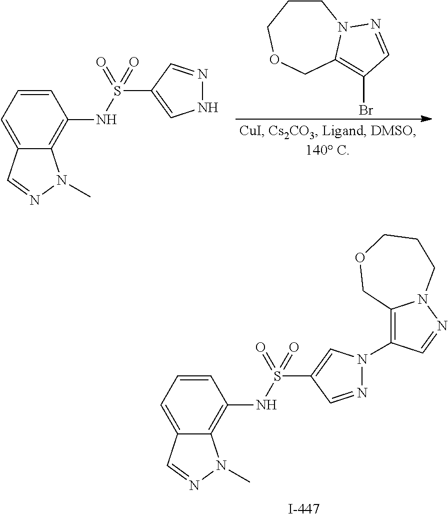 [SMILES: Cn1ncc2cccc(NS(=O)(=O)c3cn[nH]c3)c21 ; Brc1cnn2c1COCCC2 ; Cn1ncc2cccc(NS(=O)(=O)c3cnn(-c4cnn5c4COCCC5)c3)c21]

To a stirred solution of N-(1-methylindazol-7-yl)-1H-pyrazole-4-sulfonamide (107.31 mg, 0.386 mmol, 1.2 equiv) and 3-bromo-4H,6H,7H,8H-pyrazolo[3,2-c] [1,4] oxazepine (70 mg, 0.322 mmol, 1.00 equiv) in DMSO (2 mL) were added (1R,2R)-1-N,2-N-dimethylcyclohexane-1,2-diamine (22.94 mg, 0.161 mmol, 0.5 equiv), CuI (30.71 mg, 0.161 mmol, 0.5 equiv) and Cs₂CO₃ (315.21 mg, 0.966 mmol, 3 equiv) in portions at room temperature under a nitrogen atmosphere. The resulting mixture was stirred for 3.5 h at 140° C. under a nitrogen atmosphere. The resulting mixture was diluted with water (20 mL). The resulting mixture was extracted with EtOAc (3×50 mL). The combined organic layers were washed with brine (1×10 mL) and dried over anhydrous Na₂SO₄. The resulting mixture was concentrated under reduced pressure. The residue was purified by reversed-phase flash chromatography with the following conditions: column: C18 silica gel; mobile phase: MeCN in water (0.1% FA), 40% to 55% gradient in 10 min; detector: UV 254 nm. The resulting mixture was concentrated under reduced pressure. This resulted in N-(1-methylindazol-7-yl)-1-{4H,6H,7H,8H-pyrazolo[3,2-c] [1,4] oxazepin-3-yl}pyrazole-4-sulfonamide (I-447, 38.8 mg, 28.90%) as an off-white solid. LCMS (ES, m/z): [M+H]=414; ¹H NMR: (400 MHz, DMSO-d₆) δ 1.91-1.93 (m, 2H), 3.97-4.03 (m, 2H), 4.27 (s, 3H), 4.44-4.52 (m, 2H), 4.80 (s, 2H), 6.69 (dd, J=8.0, 1.0 Hz, 1H), 6.98 (t, J=8.0 Hz, 1H), 7.67 (d, J=8.0 Hz, 1H), 7.74 (s, 1H), 7.91 (s, 1H), 8.07 (s, 1H), 8.49 (s, 1H), 9.93 (s, 1H).

### Example 400—Synthesis of 1-(4-(1-Cyanocyclopentyl)Pyridin-2-yl)-N-(1-Methyl-1H-Indazol-7-yl)-1H-Pyrazole-4-Sulfonamide (I-448)

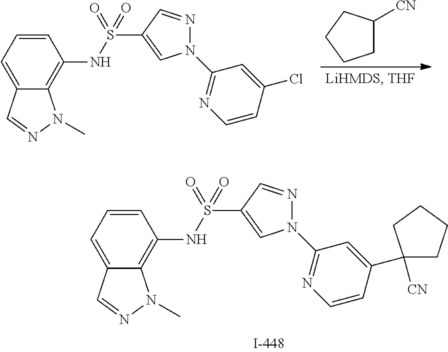 [SMILES: Cn1ncc2cccc(NS(=O)(=O)c3cnn(-c4cc(Cl)ccn4)c3)c21 ; N#CC1CCCC1 ; Cn1ncc2cccc(NS(=O)(=O)c3cnn(-c4cc(C5(C#N)CCCC5)ccn4)c3)c21]

A solution of 1-(4-chloropyridin-2-yl)-N-(1-methyl-1H-indazol-7-yl)-1H-pyrazole-4-sulfonamide (100 mg, 0.257 mmol, 1 equiv) in THE (3 mL) was treated with cyclopentanecarbonitrile (48.94 mg, 0.514 mmol, 2 equiv) for 1 min at room temperature under a nitrogen atmosphere followed by the addition of LiHMDS (0.51 mL, 0.514 mmol, 2 equiv) dropwise at 0° C. The resulting mixture was stirred for additional 2 h at 0° C. The desired product could be detected by LCMS. The resulting mixture was diluted with water (30 mL). The resulting mixture was extracted with EtOAc (3×25 mL). The combined organic layers were washed with brine (1×50 mL) and dried over anhydrous Na₂SO₄. After filtration, the filtrate was concentrated under reduced pressure. The resulting mixture was concentrated under reduced pressure. The crude product was purified by Prep-HPLC with the following conditions (column: XBridge Shield RP18 OBD column, 30*150 mm, 5 μm; mobile phase A: water (10 mmol/L NH₄HCO₃⁺0.05% NH₃H₂O), mobile phase B: ACN; flow rate: 60 mL/min; gradient: 25% B to 47% B in 7 min; wave length: 254 nm/220 nm; RT1(min): 6.9; number of runs: 4) to afford 1-(4-(1-cyanocyclopentyl)pyridin-2-yl)-N-(1-methyl-1H-indazol-7-yl)-1H-pyrazole-4-sulfonamide (I-448, 40.8 mg, 34.67%) as a white solid. LCMS (ES, m/z): [M+H]⁺ 448; ¹H NMR: (400 MHz, methanol-d₄) δ 2.03-2.10 (m, 4H), 2.19-2.26 (m, 2H), 2.48-2.56 (m, 2H), 4.38 (s, 3H), 6.74 (dd, J=7.2, 1.2 Hz, 1H), 6.96 (t, J=8.0 Hz, 1H), 7.54 (dd, J=1.8, 5.4 Hz, 1H), 7.69 (dd, J=1.2, 8.2 Hz, 1H), 7.88 (s, 1H), 8.02 (s, 1H), 8.17 (d, J=1.8 Hz, 1H), 8.48 (d, J=5.2 Hz, 1H), 8.80 (s, 1H).

### Example 401—Synthesis of 1-(4-(3-CYANO-3-Methylazetidin-1-yl)Pyridin-2-yl)-N-(1-Methyl-1H-Indazol-7-yl)-1H-Pyrazole-4-Sulfonamide (I-450)

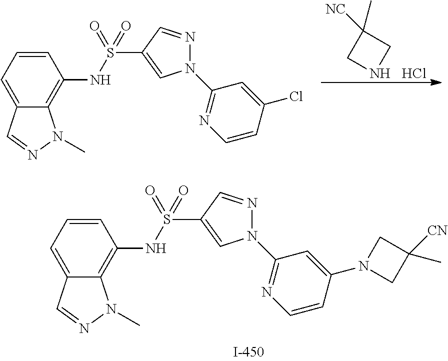

A solution of 3-methylazetidine-3-carbonitrile hydrochloride (195 mg, 1.471 mmol, 4.97 equiv) in DMSO (10 mL) was treated with Cs₂CO₃ (485 mg, 1.489 mmol, 5.03 equiv) for 5 min at room temperature under a nitrogen atmosphere followed by the addition of 1-(4-chloropyridin-2-yl)-N-(1-methylindazol-7-yl) pyrazole-4-sulfonamide (115 mg, 0.296 mmol, 1 equiv) in portions at room temperature. The resulting mixture was stirred for 15 h at 140° C. under a nitrogen atmosphere. The residue was purified by reversed-phase flash chromatography with the following conditions: column: C18 silica gel; mobile phase: MeCN in water (0.1% FA), 42% to 45% gradient in 20 min; detector: UV 254 nm. This resulted in 1-(4-(3-cyano-3-methylazetidin-1-yl)pyridin-2-yl)-N-(1-methyl-1H-indazol-7-yl)-1H-pyrazole-4-sulfonamide (I-450, 14.1 mg, 10.41%) as a white solid. LCMS (ES, m/z): [M+1]⁺=449; ¹H NMR: (400 MHz, CDCl₃) δ 1.82 (s, 3H), 4.03 (d, J=8.0 Hz, 2H), 4.41 (s, 3H), 4.47 (d, J=8.0 Hz, 2H), 6.29 (d, J=5.6 Hz, 1H), 6.65 (s, 1H), 6.78 (d, J=7.2 Hz, 1H), 6.91-6.98 (m, 2H), 7.69 (d, J=8.0 Hz, 1H), 7.72 (s, 1H), 8.00 (s, 1H), 8.09 (d, J=6.0 Hz, 1H), 9.04 (s, 1H).

### Example 402—Synthesis of 1-(7,8-Dihydro-4H,6H-Pyrazolo[5,1-C][1,4]Oxazepin-3-yl)-N-(6-Methoxy-1-Methyl-1H-Indazol-7-yl)-1H-Pyrazole-4-Sulfonamide (I-455)

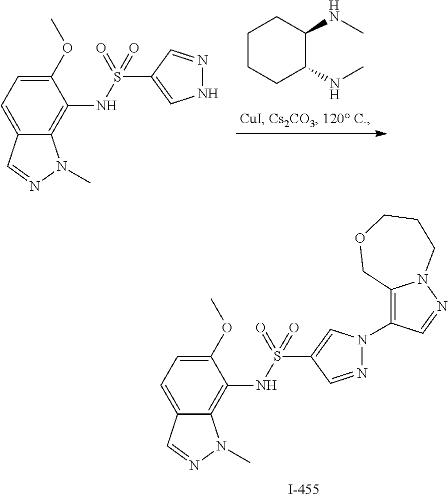

To a stirred solution of N-(6-methoxy-1-methylindazol-7-yl)-1H-pyrazole-4-sulfonamide (70 mg, 0.228 mmol, 1 equiv) and 3-bromo-4H,6H,7H,8H-pyrazolo[3,2-c] [1,4]oxazepine (59.33 mg, 0.274 mmol, 1.2 equiv) in DMSO (1 mL) were added (1R,2R)-1-N,2-N-dimethylcyclohexane-1,2-diamine (32.40 mg, 0.228 mmol, 1 equiv), CuI (43.38 mg, 0.228 mmol, 1 equiv) and Cs₂CO₃ (222.63 mg, 0.684 mmol, 3 equiv). The resulting mixture was stirred for 2 h at 120° C. under a nitrogen atmosphere. The reaction was quenched with water at room temperature. The resulting mixture was extracted with EtOAc (3×50 mL). The combined organic layers were washed with brine (1×50 mL) and dried over anhydrous Na₂SO₄. After filtration, the filtrate was concentrated under reduced pressure. The crude product (60 mg) was purified by Prep-HPLC with the following conditions (column: XBridge Prep OBD C18 column, 30*150 mm, 5 μm; mobile phase A: water (10 mmol/L NH₄HCO₃)+0.05% NH₃·H₂O, mobile phase B: ACN; flow rate: 60 mL/min; gradient: 11% B to 38% B in 7 min; wave length: 254 nm/220 nm; RT1(min): 7.6) to afford N-(6-methoxy-1-methylindazol-7-yl)-1-{4H,6H,7H,8H-pyrazolo[3,2-c][1,4]oxazepin-3-yl}pyrazole-4-sulfonamide (I-455, 37.6 mg, 36.85%) as an off-white solid. LCMS (ES, m/z): [M+H]⁺=444; ¹H NMR: (400 MHz, DMSO-d₆) δ 1.88 (h, J=4.6 Hz, 2H), 3.36 (s, 3H), 4.02-3.95 (m, 2H), 4.23 (s, 3H), 4.51-4.44 (m, 2H), 4.81 (s, 2H), 6.88 (d, J=8.9 Hz, 1H), 7.67 (d, J=8.8 Hz, 1H), 7.73 (s, 1H), 7.85 (s, 1H), 7.97 (s, 1H), 8.42 (s, 1H), 9.61 (s, 1H).

### Example 403—Synthesis of 1-(4-(1-Cyanocyclobutyl)Pyridin-2-yl)-N-(6-Methoxy-1-Methyl-1H-Indazol-7-yl)-1H-Pyrazole-4-Sulfonamide (I-457)

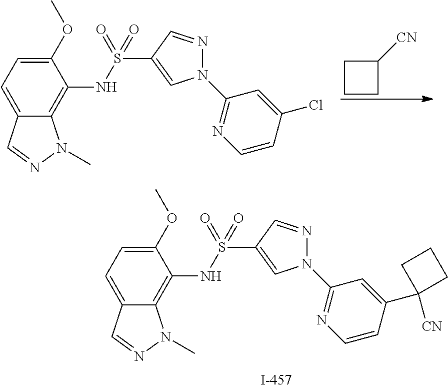

To a solution of 1-(4-chloropyridin-2-yl)-N-(6-methoxy-1-methylindazol-7-yl)pyrazole-4-sulfonamide (100 mg, 0.239 mmol, 1 equiv) and cyclobutanecarbonitrile (58.10 mg, 0.717 mmol, 3 equiv) in THE (1 mL) at 0° C. was added LiHMDS (1.0 M solution in THF, 0.71 mL, 0.717 mmol, 3 equiv) dropwise. After addition, the mixture was stirred for 3 hours at 0° C. The desired product could be detected by LCMS. The reaction was quenched by the addition of water (30 mL) at room temperature. The resulting mixture was extracted with CH₂Cl₂ (3×50 mL). The combined organic layers were washed with brine (1×20 mL) and dried over anhydrous Na₂SO₄. After filtration, the filtrate was concentrated under reduced pressure. The crude product (55 mg) was purified by Prep-HPLC with the following conditions (column: XBridge Prep OBD C18 column, 19*250 mm, 5 μm; mobile phase A: water (10 mmol/L NH₄HCO₃+0.05% NH₃·H₂O), mobile phase B: MeOH; flow rate: 25 mL/min; gradient: 45% B to 65% B in 10 min; wave length: 254 nm/220 nm; RT1(min): 9.58; number of runs: 5) to afford 1-[4-(1-cyanocyclobutyl) pyridin-2-yl]-N-(6-methoxy-1-methylindazol-7-yl)pyrazole-4-sulfonamide (I-457, 19.9 mg, 17.78%) as a white solid. LCMS (ES, m/z): [M+H]⁺=464; ¹H NMR (400 MHz, methanol-d₄) δ 2.18 (dtt, J=4.6, 4.6, 9.2, 9.2, 11.6 Hz, 1H), 2.48 (dq, J=8.8, 8.8, 8.8, 11.7 Hz, 1H), 2.71-2.81 (m, 2H), 2.81-2.91 (m, 2H), 3.40 (s, 3H), 4.34 (s, 3H), 6.83 (d, J=8.8 Hz, 1H), 7.52 (dd, J=1.8, 5.2 Hz, 1H), 7.66 (d, J=8.8 Hz, 1H), 7.82-7.91 (m, 1H), 7.93 (s, 1H), 8.10 (d, J=1.8 Hz, 1H), 8.43-8.56 (m, 1H), 8.75 (d, J=0.8 Hz, 1H).

### Example 404—Synthesis of (S)—N-(6-Methoxy-1-Methyl-1H-Indazol-7-yl)-1-(4-(1-(Piperidin-1-yl)Ethyl)Pyridin-2-yl)-1H-Pyrazole-4-Sulfonamide (I-462)

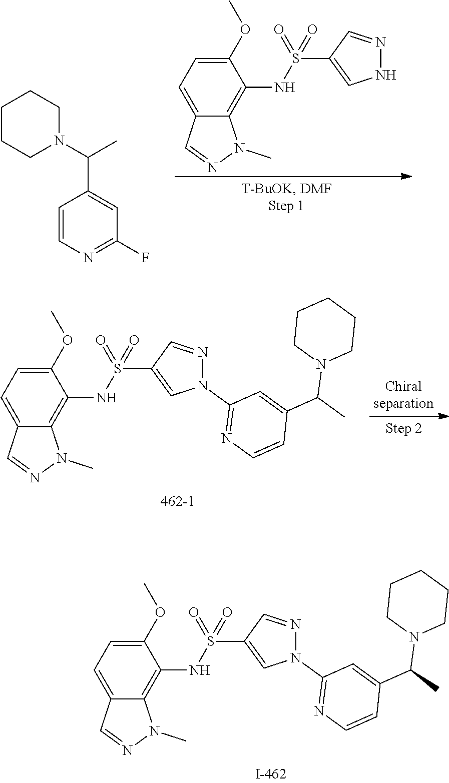 [SMILES: CC(c1ccnc(F)c1)N1CCCCC1 ; COc1ccc2cnn(C)c2c1NS(=O)(=O)c1cn[nH]c1 ; COc1ccc2cnn(C)c2c1NS(=O)(=O)c1cnn(-c2cc(C(C)N3CCCCC3)ccn2)c1 ; COc1ccc2cnn(C)c2c1NS(=O)(=O)c1cnn(-c2cc([C@H](C)N3CCCCC3)ccn2)c1]

Step 1: Synthesis of 462-1: To a stirred solution of 2-fluoro-4-[1-(piperidin-1-yl)ethyl]pyridine (140 mg, 0.672 mmol, 1 equiv) and N-(6-methoxy-1-methylindazol-7-yl)-1H-pyrazole-4-sulfonamide (247.89 mg, 0.806 mmol, 1.2 equiv) in DMF (5 mL) was added t-BuOK (226.28 mg, 2.016 mmol, 3 equiv) in portions at room temperature under a nitrogen atmosphere. The resulting mixture was stirred for 24 h at 100° C. under a nitrogen atmosphere. The resulting mixture was diluted with water (50 mL). The resulting mixture was extracted with EtOAc (3×40 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 65% gradient in 10 min; detector: UV 254 nm. The resulting mixture was concentrated under reduced pressure. This resulted in N-(6-methoxy-1-methylindazol-7-yl)-1-{4-[(1S)-1-(piperidin-1-yl)ethyl]pyridin-2-yl}pyrazole-4-sulfonamide (462-1, 48.7 mg, 11.61%) as an off-white solid.

Step 2: (S)—N-(6-methoxy-1-methyl-1H-indazol-7-yl)-1-(4-(1-(piperidin-1-yl)ethyl)pyridin-2-yl)-1H-pyrazole-4-sulfonamide (I-462): 462-1 (48.7 mg) was purified by Prep-Chiral-HPLC with the following conditions (column: CHIRALPAKIG3; mobile phase A: hex (0.2% DEA): (EtOH:MeOH=1:1)=65:35; flow rate: 1 mL/min; gradient: isocratic; injection volume: 3 mL; the second peak was product) to afford (S)—N-(6-methoxy-1-methyl-1H-indazol-7-yl)-1-(4-(1-(piperidin-1-yl)ethyl)pyridin-2-yl)-1H-pyrazole-4-sulfonamide (I-462, 12.9 mg, 33.33%) as an off-white solid. LCMS (ES, m/z): [M+H]⁺=496; ¹H NMR: (400 MHz, DMSO-d₆) δ 1.34 (t, J=8.6 Hz, 5H), 1.50 (t, J=5.8 Hz, 4H), 2.35 (d, J=16.8 Hz, 4H), 3.34 (s, 3H), 3.63 (d, J=6.8 Hz, 1H), 4.25 (s, 3H), 6.88 (d, J=8.8 Hz, 1H), 7.41 (dd, J=5.0, 1.4 Hz, 1H), 7.68 (d, J=8.8 Hz, 1H), 7.91 (s, 1H), 7.99 (d, J=6.2 Hz, 2H), 8.45 (d, J=5.0 Hz, 1H), 8.71 (s, 1H).

### Example 405—Synthesis of 1-(6,7-Dihydro-4H-Pyrazolo[5,1-C][1,4]Oxazin-3-yl)-N-(1-Methyl-1H-Indazol-7-yl)-1H-Pyrazole-4-Sulfonamide (I-464)

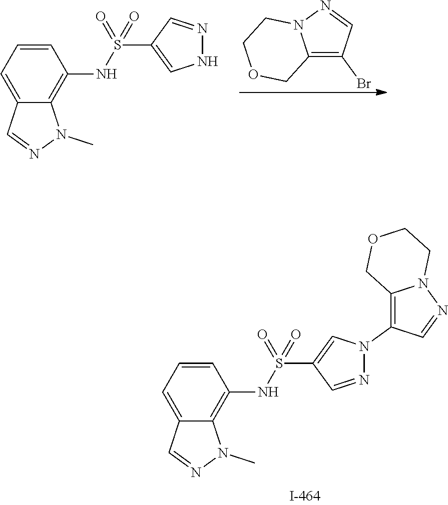

A solution of N-(1-methylindazol-7-yl)-1H-pyrazole-4-sulfonamide (150 mg, 0.541 mmol, 1 equiv), 3-bromo-4H,6H,7H-pyrazolo[3,2-c][1,4]oxazine (150 mg, 0.739 mmol, 1.37 equiv), cyclohexane-1,2-diamine (30 mg, 0.271 mmol, 0.5 equiv), CuI (50 mg, 0.271 mmol, 0.5 equiv) and Cs₂CO₃ (530 mg, 1.623 mmol, 3 equiv) in DMSO (3 mL) was stirred for 2 h at 80° C. under a nitrogen atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The crude product (100 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 11% B to 36% B in 8 min; wave length: 220 nm; RT1(min): 7.43) to afford N-(1-methylindazol-7-yl)-1-{4H,6H,7H-pyrazolo[3,2-c][1,4]oxazin-3-yl}pyrazole-4-sulfonamide (I-464, 58.8 mg, 27.11%) as an off-white solid. LCMS (ES, m/z): [M+1]⁺=400; ¹H NMR: 1H NMR (400 MHz, DMSO-d₆) 4.09 (dd, J=6.2, 4.2 Hz, 2H), 4.16 (t, J=5.2 Hz, 2H), 4.27 (s, 3H), 4.98 (s, 2H), 6.68 (d, J=7.4 Hz, 1H), 6.99 (t, J=7.8 Hz, 1H), 7.69 (d, J=8.0 Hz, 1H), 7.89 (s, 1H), 8.00 (s, 1H), 8.08 (s, 1H), 8.62 (s, 1H), 9.91 (s, 1H).

### Example 406—Synthesis of 1-(6,7-Dihydro-5H-Pyrazolo[5,1-B][1,3]Oxazin-3-yl)-N-(1-Methyl-1H-Indazol-7-yl)-1H-Pyrazole-4-Sulfonamide (I-465)

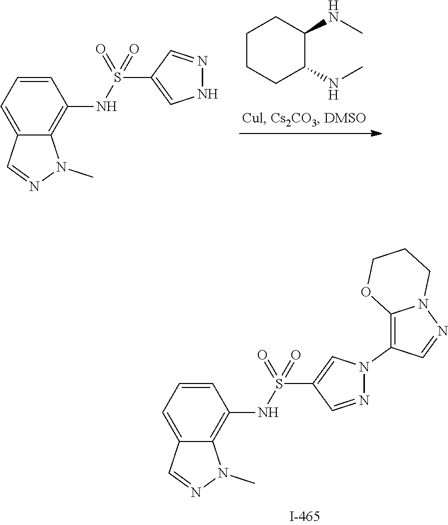

To a stirred solution of N-(1-methylindazol-7-yl)-1H-pyrazole-4-sulfonamide (100 mg, 0.361 mmol, 1 equiv) and 3-bromo-5H,6H,7H-pyrazolo[3,2-b][1,3]oxazine (87.86 mg, 0.433 mmol, 1.2 equiv) in DMSO (5 mL) were added cyclohexane-1,2-diamine (41.18 mg, 0.361 mmol, 1 equiv), CuI (68.68 mg, 0.361 mmol, 1 equiv) and Cs₂CO₃ (352.49 mg, 1.083 mmol, 3 equiv). The resulting mixture was stirred for 2 h at 120° C. under a nitrogen atmosphere. The reaction was quenched with water at room temperature. The resulting mixture was extracted with EtOAc (3×80 mL). The combined organic layers were washed with brine (1×80 mL) and dried over anhydrous Na₂SO₄. After filtration, the filtrate was concentrated under reduced pressure. The crude product (140 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 5% B to 30% B in 8 min; wave length: 220 nm; RT1(min): 7.07) to afford N-(1-methylindazol-7-yl)-1-{5H,6H,7H-pyrazolo[3,2-b][1,3]oxazin-3-yl}pyrazole-4-sulfonamide (I-465, 69.6 mg, 48.17%) as an off-white solid. LCMS (ES, m/z): [M+H]⁺=400; ¹H NMR: (400 MHz, DMSO-d₆) δ 9.89 (s, 1H), 8.17 (d, J=0.7 Hz, 1H), 8.08 (s, 1H), 7.82 (d, J=0.7 Hz, 1H), 7.72-7.65 (m, 2H), 7.00 (dd, J=8.1, 7.3 Hz, 1H), 6.68 (dd, J=7.3, 1.0 Hz, 1H), 4.44-4.37 (m, 2H), 4.26 (s, 3H), 4.14 (t, J=6.1 Hz, 2H), 2.28-2.18 (m, 2H).

### Example 407—Synthesis of N-(1-Methyl-1H-Indazol-7-yl)-1-(5-Methyl-4,5,6,7-Tetrahydropyrazolo[1,5-A]Pyrazin-3-yl)-1H-Pyrazole-4-Sulfonamide (I-466)

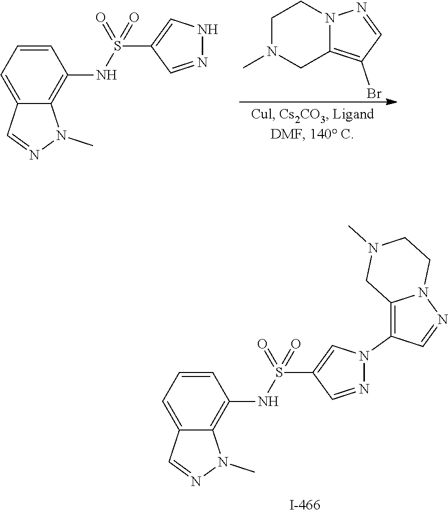

To a stirred solution of 3-bromo-5-methyl-4H,6H,7H-pyrazolo[1,5-a]pyrazine (124.68 mg, 0.576 mmol, 2 equiv) and N-(1-methylindazol-7-yl)-1H-pyrazole-4-sulfonamide (80 mg, 0.288 mmol, 1.00 equiv) in DMF (2 mL) were added (1R,2R)-1-N,2-N-dimethylcyclohexane-1,2-diamine (20.52 mg, 0.144 mmol, 0.5 equiv), CuI (27.47 mg, 0.144 mmol, 0.5 equiv) and Cs₂CO₃ (281.99 mg, 0.864 mmol, 3 equiv) at room temperature under a nitrogen atmosphere. The resulting mixture was stirred overnight at 140° C. under a nitrogen atmosphere. The resulting mixture was diluted with water (10 mL). The resulting mixture was extracted with EtOAc (3×5 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The product (40 mg) was then purified by Prep-HPLC with the following conditions (column: ER Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 5% B to 30% B in 9 min; wave length: 220 nm; RT1(min): 7.93) to afford 1-{5-methyl-4H,6H,7H-pyrazolo[1,5-a]pyrazin-3-yl}-N-(1-methylindazol-7-yl)pyrazole-4-sulfonamide (I-466, 13.6 mg, 11.35%) as an off-white solid. LCMS (ES, m/z): [M+1]=413; ¹H NMR: ¹H NMR (400 MHz, DMSO-d₆) δ 2.42 (s, 3H), 2.86 (t, J=5.6 Hz, 2H), 3.73 (s, 2H), 4.13 (t, J=5.6 Hz, 2H), 4.28 (s, 3H), 6.70 (d, J=7.2 Hz, 1H), 6.98 (t, J=7.6 Hz, 1H), 7.63 (d, J=7.6 Hz, 1H), 7.88 (s, 1H), 7.91 (s, 1H), 8.05 (s, 1H), 8.50 (s, 1H), 9.86 (s, 1H).

### Example 408—Synthesis of 1-(6,7-Dihydro-5H-Pyrazolo[5,1-B][1,3]Oxazin-3-yl)-N-(6-Methoxy-1-Methyl-1H-Indazol-7-yl)-1H-Pyrazole-4-Sulfonamide (I-467)

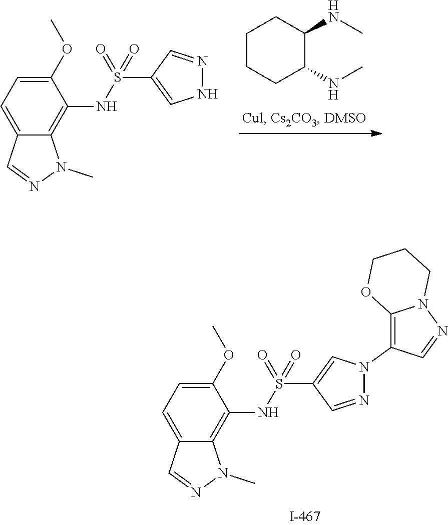

To a stirred solution of N-(6-methoxy-1-methylindazol-7-yl)-1H-pyrazole-4-sulfonamide (100 mg, 0.325 mmol, 1 equiv) and 3-bromo-5H,6H,7H-pyrazolo[3,2-b][1,3]oxazine (79.28 mg, 0.390 mmol, 1.2 equiv) in DMSO (5 mL) were added cyclohexane-1,2-diamine (37.16 mg, 0.325 mmol, 1 equiv), CuI (61.97 mg, 0.325 mmol, 1 equiv) and Cs₂CO₃ (318.05 mg, 0.975 mmol, 3 equiv). The resulting mixture was stirred for 2 h at 120° C. under a nitrogen atmosphere. The reaction was quenched with water at room temperature. The resulting mixture was extracted with EtOAc (3×80 mL). The combined organic layers were washed with brine (1×80 mL) and dried over anhydrous Na₂SO₄. After filtration, the filtrate was concentrated under reduced pressure. The crude product (80 mg) was purified by Prep-HPLC with the following conditions (column: XBridge Shield RP18 OBD column, 30*150 mm, 5 μm; mobile phase A: water (10 mmol/L NH₄HCO₃+0.05% NH₃H₂O), mobile phase B: ACN; flow rate: 60 mL/min; gradient: 15% B to 35% B in 7 min; wave length: 254 nm/220 nm; RT1(min): 6.7; number of runs: 3) to afford N-(6-methoxy-1-methylindazol-7-yl)-1-{5H,6H,7H-pyrazolo[3,2-b] [1,3] oxazin-3-yl} pyrazole-4-sulfonamide (I-467, 34.5 mg, 24.59%) as an off-white solid. LCMS (ES, m/z): [M+H]⁺=430; ¹H NMR: (400 MHz, DMSO-d₆) δ 9.56 (s, 1H), 8.08 (s, 1H), 7.97 (s, 1H), 7.74 (s, 1H), 7.66 (d, J=9.3 Hz, 2H), 6.89 (d, J=8.9 Hz, 1H), 4.44-4.31 (m, 2H), 4.22 (s, 3H), 4.18-4.04 (m, 2H), 3.38 (s, 3H), 2.29-2.12 (m, 2H).

### Example 409—Synthesis of N-(6-Methoxy-1-Methyl-1H-Indazol-7-yl)-1-(5-Methyl-4,5,6,7-Tetrahydropyrazolo[1,5-A]Pyrazin-3-yl)-1H-Pyrazole-4-Sulfonamide (I-468)

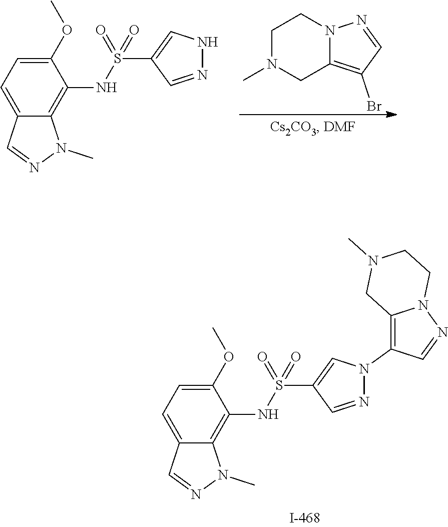

To a stirred solution of N-(6-methoxy-1-methylindazol-7-yl)-1H-pyrazole-4-sulfonamide (85 mg, 0.277 mmol, 1.00 equiv), Cs₂CO₃ (270.34 mg, 0.831 mmol, 3 equiv) and 3-bromo-5-methyl-4H,6H,7H-pyrazolo[1,5-a]pyrazine (149.41 mg, 0.693 mmol, 2.5 equiv) in DMF (3 mL) were added CuI (26.34 mg, 0.139 mmol, 0.5 equiv) and (1R,2R)-1-N,2-N-dimethylcyclohexane-1,2-diamine (39.34 mg, 0.277 mmol, 1 equiv) dropwise at room temperature under a nitrogen atmosphere. The resulting mixture was stirred overnight at 140° C. The resulting mixture was extracted with EtOAc (3×10 mL). The combined organic layers were washed with water (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 15 min; detector: UV 254 nm. This resulted in N-(6-methoxy-1-methylindazol-7-yl)-1-{5-methyl-4H,6H,7H-pyrazolo[1,5-a]pyrazin-3-yl}pyrazole-4-sulfonamide (I-468, 15.6 mg, 12.48%) as an off-white solid. LCMS (ES, m/z): [M+1]⁺=443; ¹H NMR: ¹H NMR (400 MHz, DMSO-d₆) δ 2.42 (s, 3H), 2.87 (t, J=5.5 Hz, 2H), 3.35 (s, 3H), 3.75 (s, 2H), 4.13 (t, J=5.6 Hz, 2H), 4.23 (s, 3H), 6.89 (d, J=8.8 Hz, 1H), 7.67 (d, J=8.8 Hz, 1H), 7.82 (d, J=0.8 Hz, 1H), 7.92 (s, 1H), 7.97 (s, 1H), 8.47 (d, J=0.8 Hz, 1H), 9.56 (s, 1H).

### Example 410—Synthesis of 1-(4-(1-Cyanocyclopropyl)Pyridin-2-yl)-N-(6-Methoxy-1-Methyl-1H-Indazol-7-yl)-1H-Pyrazole-4-Sulfonamide (I-470)

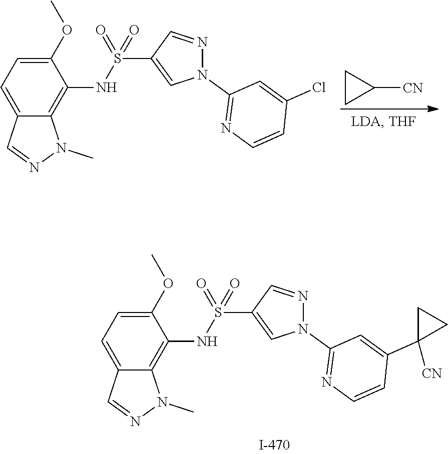

To a solution of 1-(4-chloropyridin-2-yl)-N-(6-methoxy-1-methylindazol-7-yl)pyrazole-4-sulfonamide (100 mg, 0.239 mmol, 1 equiv) and cyclopropyl cyanide (80.09 mg, 1.195 mmol, 5 equiv) in THF (2 mL) at 0° C. was added LDA (2 M in THF, 0.2 mL, 0.143 mmol, 3 equiv) dropwise. After the addition, the mixture was stirred for 3 hours at 0° C. The desired product could be detected by LCMS. The resulting mixture was diluted with water (10 mL). The resulting mixture was extracted with CH₂Cl₂ (3×20 mL). The combined organic layers were washed with brine (1×10 mL) and dried over anhydrous Na₂SO₄. After filtration, the filtrate was concentrated under reduced pressure. The crude product (70 mg) was purified by Prep-HPLC with the following conditions (column: Xselect CSH C18 OBD column 30*150 mm 5 μm; mobile phase A: water (0.1% FA), mobile phase B: MeOH; flow rate: 60 mL/min; gradient: 45% B to 55% B in 9 min; wave length: 254 nm/220 nm; RT1(min): 8.3) to afford 1-[4-(1-cyanocyclopropyl)pyridin-2-yl]-N-(6-methoxy-1-methylindazol-7-yl)pyrazole-4-sulfonamide (I-470, 8.2 mg, 7.43%) as a white solid. LCMS (ES, m/z): [M+H]=450; ¹H NMR: (400 MHz, methanol-d₄) δ 1.70-1.78 (m, 2H), 1.88-2.00 (m, 2H), 3.40 (s, 3H), 4.34 (s, 3H), 6.84 (d, J=8.8 Hz, 1H), 7.27 (dd, J=5.2, 1.8 Hz, 1H), 7.66 (d, J=8.8 Hz, 1H), 7.87 (s, 1H), 7.91-7.99 (m, 2H), 8.42 (d, J=5.2 Hz, 1H), 8.73 (s, 1H).

### Example 411—Synthesis of 1-(2-(4-(N-(1-Methyl-1H-Indazol-7-yl)Sulfamoyl)-1H-Pyrazol-1-yl)Pyridin-4-yl)Azetidine-3-Carboxamide (I-471)

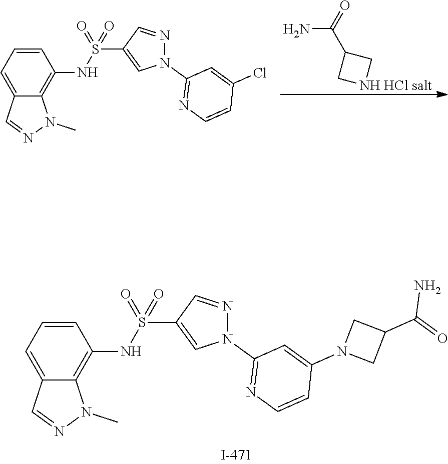

To a stirred mixture of 1-(4-chloropyridin-2-yl)-N-(1-methylindazol-7-yl)pyrazole-4-sulfonamide (50 mg, 0.129 mmol, 1 equiv) and azetidine-3-carboxamide (32.19 mg, 0.323 mmol, 2.5 equiv) in DMSO (2 mL) were added Cs₂CO₃ (125.69 mg, 0.387 mmol, 3 equiv) in portions at 120° C. under a nitrogen atmosphere. The resulting mixture was stirred overnight at 120° C. The crude product (50 mg) was purified by Prep-HPLC with the following conditions (column: XBridge Prep OBD C18 column, 30*150 mm, 5 μm; mobile phase A: water(0.1% FA), mobile phase B: ACN; flow rate: 60 mL/min; gradient: 13% B to 34% B in 9 min; wave length: 254 nm/220 nm; RT1(min): 9.83; number of runs: 5) to afford 1-(2-(4-(N-(1-methyl-1H-indazol-7-yl)sulfamoyl)-1H-pyrazol-1-yl)pyridin-4-yl)azetidine-3-carboxamide (I-471, 7.8 mg, 13.27%) as a white solid. LCMS (ES, m/z): [M+1]⁺=453; ¹H NMR: ¹H NMR (400 MHz, DMSO-d₆) δ3.51 (td, J=8.6, 4.2 Hz, 1H), 4.01 (dd, J=8.1, 5.8 Hz, 2H), 4.15 (t, J=8.2 Hz, 2H), 4.28 (s, 3H), 6.42 (dd, J=5.7, 2.2 Hz, 1H), 6.68 (d, J=7.2 Hz, 1H), 6.85 (d, J=2.2 Hz, 1H), 6.98 (t, J=7.8 Hz, 1H), 7.11 (s, 1H), 7.54-7.58 (m, 1H), 7.70 (dd, J=8.1, 0.8 Hz, 1H), 7.94 (s, 1H), 8.04 (d, J=5.6 Hz, 1H), 8.09 (s, 1H), 8.72 (s, 1H), 9.99 (s, 1H).

### Example 412—Synthesis of 1-(5,6-Dihydro-8H-Imidazo[2,1-C][1,4]Oxazin-3-yl)-N-(1-Methyl-1H-Indazol-7-yl)-1H-Pyrazole-4-Sulfonamide (I-473)

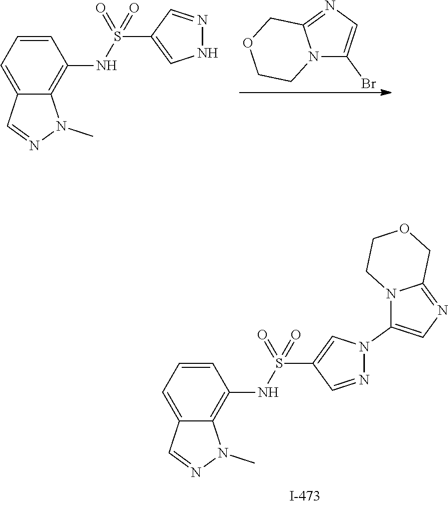 [SMILES: Cn1ncc2cccc(NS(=O)(=O)c3cn[nH]c3)c21 ; Brc1cnc2n1CCOC2 ; Cn1ncc2cccc(NS(=O)(=O)c3cnn(-c4cnc5n4CCOC5)c3)c21]

To a stirred mixture of N-(1-methylindazol-7-yl)-1H-pyrazole-4-sulfonamide (100 mg, 0.361 mmol, 1 equiv) and 3-bromo-5H,6H,8H-imidazo[2,1-c][1,4]oxazine (73.22 mg, 0.361 mmol, 1 equiv) in DMSO (10 mL) were added CuI (34.34 mg, 0.180 mmol, 0.5 equiv), (1R,2R)-1-N,2-N-dimethylcyclohexane-1,2-diamine (25.65 mg, 0.180 mmol, 0.5 equiv) and Cs₂CO₃ (352.49 mg, 1.083 mmol, 3 equiv) in portions at 140° C. under a nitrogen atmosphere. The resulting mixture was stirred for 2 h at 140° C. under a nitrogen atmosphere. The crude product (100 mg) was purified by Prep-HPLC with the following conditions (column: XBridge Prep OBD C18 column, 19*250 mm, 5 μm; mobile phase A: water (10 mmol/L NH₄HCO₃+0.05% NH₃H₂O), mobile phase B: MeOH; flow rate: 25 mL/min; gradient: 23% B to 47% B in 10 min; wave length: 254 nm/220 nm; RT1(min): 9.65; number of runs: 7) to afford 1-(5,6-dihydro-8H-imidazo[2,1-c][1,4]oxazin-3-yl)-N-(1-methyl-1H-indazol-7-yl)-1H-pyrazole-4-sulfonamide (1-473, 30.7 mg, 20.65%) as a white solid. LCMS (ES, m/z): [M+1]⁺=400; ¹H NMR:¹H NMR (400 MHz, DMSO-d₆) δ 3.82 (t, J=5.2 Hz, 2H) 4.02 (dd, J=5.8, 4.6 Hz, 2H), 4.26 (s, 3H), 4.77 (s, 2H), 6.68-6.74 (m, 1H), 7.02 (t, J=7.6 Hz, 1H), 7.18 (s, 1H), 7.69 (d, J=8.0 Hz, 1H), 8.02 (s, 1H), 8.08 (s, 1H), 8.50 (s, 1H), 9.99 (s, 1H).

### Example 413—Synthesis of 1-(6,7-Dihydro-4H-Pyrazolo[5,1-C][1,4]Oxazin-3-yl)-N-(6-Methoxy-1-Methyl-1H-Indazol-7-yl)-1H-Pyrazole-4-Sulfonamide (I-474)

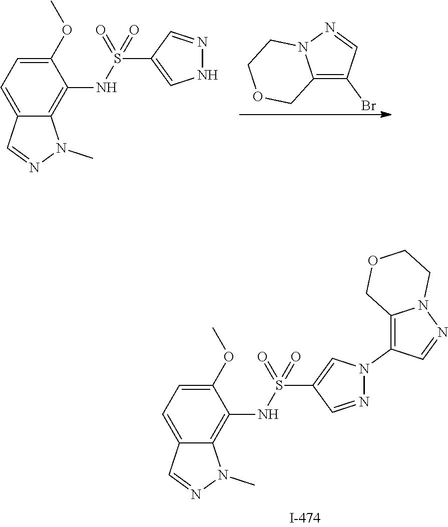 [SMILES: COc1ccc2cnn(C)c2c1NS(=O)(=O)c1cn[nH]c1 ; Brc1cnn2c1COCC2 ; COc1ccc2cnn(C)c2c1NS(=O)(=O)c1cnn(-c2cnn3c2COCC3)c1]

A solution of N-(6-methoxy-1-methylindazol-7-yl)-1H-pyrazole-4-sulfonamide (150 mg, 0.488 mmol, 1 equiv), 3-bromo-4H,6H,7H-pyrazolo[3,2-c][1,4]oxazine (120 mg, 0.591 mmol, 1.21 equiv), CuI (46.48 mg, 0.244 mmol, 0.5 equiv), cyclohexane-1,2-diamine (27.87 mg, 0.244 mmol, 0.5 equiv) and Cs₂CO₃ (477.07 mg, 1.464 mmol, 3 equiv) in DMSO (3 mL) was stirred for 2 h at 80° C. under a nitrogen atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The crude product (100 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 11% B to 36% B in 8 min; wave length: 220 nm; RT1(min): 7.43) to afford N-(6-methoxy-1-methylindazol-7-yl)-1-{4H,6H,7H-pyrazolo[3,2-c][1,4]oxazin-3-yl}pyrazole-4-sulfonamide (I-474, 31.2 mg, 14.71%) as an off-white solid. LCMS (ES, m/z): [M+1]⁺=430; ¹H NMR (400 MHz, DMSO-d₆) δ 3.34 (s, 3H), 4.10 (t, J=5.2 Hz, 2H), 4.16 (t, J=5.2 Hz, 2H), 4.24 (s, 3H), 4.98 (s, 2H), 6.88 (d, J=8.8 Hz, 1H), 7.67 (d, J=8.8 Hz, 1H), 7.82 (s, 1H), 7.98 (d, J=2.6 Hz, 2H), 8.52 (s, 1H).

### Example 414—Synthesis of 1-(5,6-Dihydro-8H-Imidazo[2,1-C][1,4]Oxazin-3-yl)-N-(6-Methoxy-1-Methyl-1H-Indazol-7-yl)-1H-Pyrazole-4-Sulfonamide (I-475)

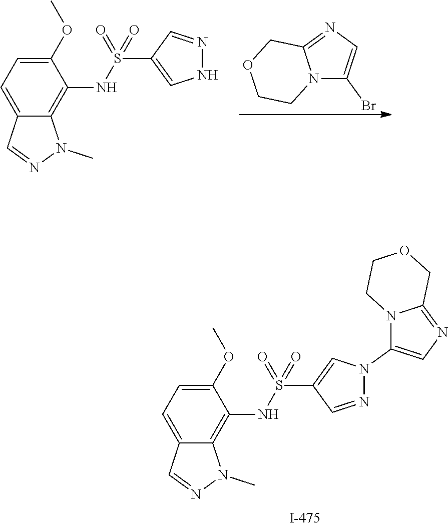 [SMILES: COc1ccc2cnn(C)c2c1NS(=O)(=O)c1cn[nH]c1 ; Brc1cnc2n1CCOC2 ; COc1ccc2cnn(C)c2c1NS(=O)(=O)c1cnn(-c2cnc3n2CCOC3)c1]

To a stirred mixture of N-(6-methoxy-1-methylindazol-7-yl)-1H-pyrazole-4-sulfonamide (100 mg, 0.325 mmol, 1 equiv) and 3-bromo-5H,6H,8H-imidazo[2,1-c][1,4]oxazine (66.07 mg, 0.325 mmol, 1 equiv) in DMSO (5 mL) were added CuI (30.98 mg, 0.163 mmol, 0.5 equiv), (1R,2R)—N1,N2-dimethylcyclohexane-1,2-diamine (23.14 mg, 0.163 mmol, 0.5 equiv) and Cs₂CO₃ (318.05 mg, 0.975 mmol, 3 equiv) in portions at 140° C. under a nitrogen atmosphere. The resulting mixture was stirred for 2 h at 140° C. under a nitrogen atmosphere. The crude product (100 mg) was purified by Prep-HPLC with the following conditions (column: XBridge Prep OBD C18 column, 19*250 mm, 5 μm; mobile phase A: water (10 mmol/L NH₄HCO₃+0.05% NH₃H₂O), mobile phase B: MeOH; flow rate: 25 mL/min; gradient: 23% B to 47% B in 10 min; wave length: 254 nm/220 nm; RT1(min): 9.65; number of runs: 7) to afford 1-(5,6-dihydro-8H-imidazo[2,1-c][1,4]oxazin-3-yl)-N-(6-methoxy-1-methyl-1H-indazol-7-yl)-1H-pyrazole-4-sulfonamide (I-475, 21.8 mg, 14.96%) as a brown solid. LCMS (ES, m/z): [M+1]⁺=430; ¹H NMR (400 MHz, DMSO-d₆) δ 3.44 (s, 3H), 3.88 (t, J=5.2 Hz, 2H), 4.02 (dd, J=5.8, 4.4 Hz, 2H), 4.23 (s, 3H), 4.77 (s, 2H), 6.91 (d, J=8.8 Hz, 1H), 7.16 (s, 1H), 7.65 (d, J=8.8 Hz, 1H), 7.95 (d, J=6.8 Hz, 2H), 8.42 (s, 1H), 9.61 (s, 1H).

### Example 415—Synthesis of N-(6-(Methyoxy-D3)-1-(Methyl-D3)-1H-Indazol-7-yl)-1-(4-(Trifluoromethyl)Pyridin-2-yl)-1H-Pyrazole-4-Sulfonamide (I-476)

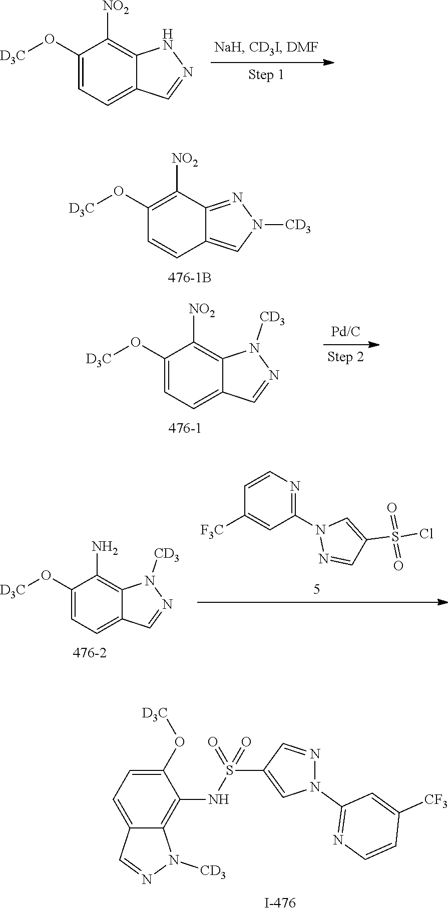 [SMILES: [2H]C([2H])([2H])Oc1ccc2cn[nH]c2c1[N+](=O)[O-] ; [2H]C([2H])([2H])Oc1ccc2cn(C([2H])([2H])[2H])nc2c1[N+](=O)[O-] ; [2H]C([2H])([2H])Oc1ccc2cnn(C([2H])([2H])[2H])c2c1[N+](=O)[O-] ; [2H]C([2H])([2H])Oc1ccc2cnn(C([2H])([2H])[2H])c2c1N ; O=S(=O)(Cl)c1cnn(-c2cc(C(F)(F)F)ccn2)c1 ; [2H]C([2H])([2H])Oc1ccc2cnn(C([2H])([2H])[2H])c2c1NS(=O)(=O)c1cnn(-c2cc(C(F)(F)F)ccn2)c1]

Step 1: Synthesis of 476-1: To a stirred solution of 6-(2H3)methoxy-7-nitro-1H-indazole (3 g, 15.292 mmol, 1 equiv) and CD₃I (3.02 mL, 45.876 mmol, 3 equiv) in DMF (30 mL) was added NaH (1.10 g, 45.876 mmol, 3 equiv) at room temperature under a nitrogen atmosphere. The resulting mixture was stirred for 2 h at room temperature under a nitrogen atmosphere. The residue was purified by silica gel column chromatography and eluted with PE/EA (7:1) to afford 6-(methoxy-d3)-1-(methyl-d3)-7-nitro-1H-indazole (476-1, 1.5 g, 41.40%) as a white solid

Step 2: Synthesis of 476-2: A solution of 6-(methoxy-d3)-1-(methyl-d3)-7-nitro-1H-indazole (1.5 g, 7.035 mmol, 1 equiv) and Pd/C (2.25 g, 21.105 mmol, 3 equiv) in MeOH (15 mL) was stirred for 3 h at room temperature under a hydrogen atmosphere. The resulting mixture was concentrated under vacuum. This resulted in 6-(2H3)methoxy-1-(2H3)methylindazol-7-amine (476-2, 1.1 g, 76.80%) as a yellow oil.

Step 3: Synthesis of N-(6-(methoxy-d3)-1-(methyl-d3)-1H-indazol-7-yl)-1-(4-(trifluoromethyl)pyridin-2-yl)-1H-pyrazole-4-sulfonamide (I-476): A solution of 6-(2H3)methoxy-1-(2H3)methyl-7-nitroindazole (300 mg, 1.407 mmol, 1 equiv) and 1-[4-(trifluoromethyl)pyridin-2-yl]pyrazole-4-sulfonyl chloride (657.74 mg, 2.111 mmol, 1.5 equiv) in pyridine (3 mL) was stirred at room temperature under a nitrogen atmosphere. The resulting mixture was stirred for 2 h at room temperature under a nitrogen atmosphere. The resulting mixture was concentrated under vacuum. The residue was purified by reversed-phase flash chromatography with the following conditions: column: C18 silica gel; mobile phase: MeCN in water (0.1% FA), 0% to 100% gradient in 20 min; detector: UV 254 nm. This resulted in N-[6-(2H3)methoxy-1-(2H3)methylindazol-7-yl]-1-[4-(trifluoromethyl)pyridin-2-yl]pyrazole-4-sulfonamide (I-476, 141.9 mg, 21.98%) as a white solid. LCMS (ES, m/z): [M+H]⁺ 459; ¹H NMR (400 MHz, DMSO-d₆) δ 6.87 (d, J=8.8 Hz, 1H), 7.66 (d, J=8.6 Hz, 1H), 7.87 (d, J=5.2 Hz, 1H), 7.97 (s, 1H), 8.07 (s, 1H), 8.19 (s, 1H), 8.77 (s, 1H), 8.82 (d, J=5.1 Hz, 1H).

### Example 416—Synthesis of N-(6-Methoxy-1-Methyl-1H-Indazol-7-yl)-1-(4-(1-Methoxy-2-Methylpropan-2-yl)Pyridin-2-yl)-1H-Pyrazole-4-Sulfonamide (I-482)

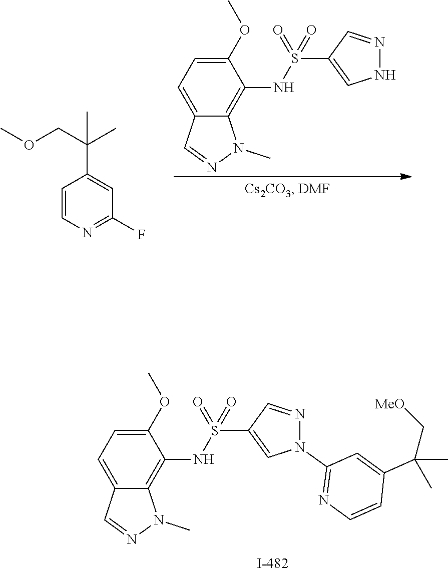 [SMILES: COCC(C)(C)c1ccnc(F)c1 ; COc1ccc2cnn(C)c2c1NS(=O)(=O)c1cn[nH]c1 ; COCC(C)(C)c1ccnc(-n2cc(S(=O)(=O)Nc3c(OC)ccc4cnn(C)c34)cn2)c1]

To a stirred mixture of 2-fluoro-4-(1-methoxy-2-methylpropan-2-yl)pyridine (50 mg, 0.273 mmol, 1 equiv) and N-(6-methoxy-1-methylindazol-7-yl)-1H-pyrazole-4-sulfonamide (83.87 mg, 0.273 mmol, 1 equiv) in DMF (3 mL) was added Cs₂CO₃ (266.74 mg, 0.819 mmol, 3 equiv) in portions at 100° C. under an air atmosphere. The resulting mixture was stirred overnight at 100° C. The crude product (60 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 19% B to 44% B in 8 min; wave length: 220 nm; RT1(min): 8.52) to afford N-(6-methoxy-1-methyl-1H-indazol-7-yl)-1-(4-(1-methoxy-2-methylpropan-2-yl)pyridin-2-yl)-1H-pyrazole-4-sulfonamide (I-482, 17.4 mg, 13.52%) as a white solid. LCMS (ES, m/z): [M+1]=471; ¹H NMR (400 MHz, chloroform-d) δ 1.37 (s, 6H), 3.32 (s, 3H), 3.44 (d, J=9.6 Hz, 5H), 4.44 (s, 3H), 6.47 (s, 1H), 6.70 (d, J=8.8 Hz, 1H), 7.26-7.32 (m, 1H), 7.63 (d, J=8.8 Hz, 1H), 7.70 (s, 1H), 7.92 (d, J=1.7 Hz, 1H), 7.97 (s, 1H), 8.32 (d, J=5.4 Hz, 1H), 8.73 (s, 1H).

### Example 417—Synthesis of N-(6-Methoxy-1-Methyl-1H-Indazol-7-yl)-1-(4-(5-Methoxyspiro[2.3]Hexan-5-yl)Pyridin-2-yl)-1H-Pyrazole-4-Sulfonamide (I-485)

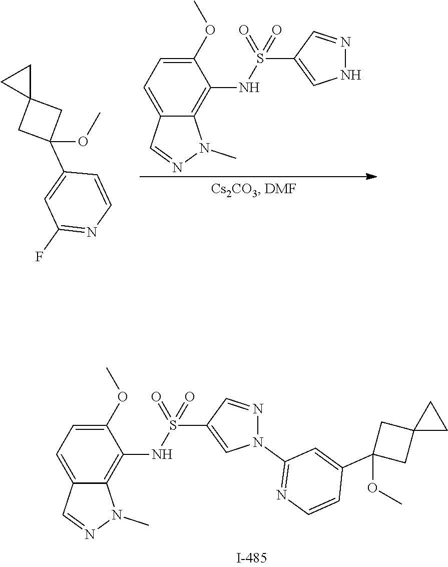 [SMILES: COC1(c2ccnc(F)c2)CC2(CC2)C1 ; COc1ccc2cnn(C)c2c1NS(=O)(=O)c1cn[nH]c1 ; COc1ccc2cnn(C)c2c1NS(=O)(=O)c1cnn(-c2cc(C3(OC)CC4(CC4)C3)ccn2)c1]

A solution of 2-fluoro-4-{5-methoxyspiro [2.3] hexan-5-yl} pyridine (85 mg, 0.410 mmol, 1 equiv) in DMF (3 mL) was treated with N-(6-methoxy-1-methylindazol-7-yl)-1H-pyrazole-4-sulfonamide (151.26 mg, 0.492 mmol, 1.2 equiv) followed by the addition of t-BuOK (138.07 mg, 1.230 mmol, 3 equiv). The resulting mixture was stirred for 2 h at 100° C. under a nitrogen atmosphere. The desired product could be detected by LCMS. The reaction was quenched with water at room temperature. The resulting mixture was extracted with EtOAc (2×80 mL). The combined organic layers were washed with brine (2×50 mL) and dried over anhydrous Na₂SO₄. After filtration, the filtrate was concentrated under reduced pressure. The crude product (80 mg) was purified by Prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 38% B to 63% B in 8 min; wave length: 220 nm; RT1(min): 7.9) to afford N-(6-methoxy-1-methylindazol-7-yl)-1-(4-{5-methoxyspiro [2.3] hexan-5-yl} pyridin-2-yl) pyrazole-4-sulfonamide (I-485, 48.6 mg, 23.96%) as an off-white solid. LCMS (ES, m/z): [M+H]=495; ¹H NMR: (400 MHz, DMSO-d₆) δ 0.56 (s, 4H), 2.33 (d, J=13.6 Hz, 2H), 2.68 (d, J=13.6 Hz, 2H), 3.06 (s, 3H), 3.35 (s, 3H), 4.25 (s, 3H), 6.88 (d, J=8.8 Hz, 1H), 7.59 (m, J=5.2, 1.6 Hz, 1H), 7.67 (d, J=8.8 Hz, 1H), 7.96-8.06 (m, 3H), 8.56 (m, J=5.1, 0.6 Hz, 1H), 8.73 (d, J=0.8 Hz, 1H), 9.80 (s, 1H).

### Example 418—Synthesis of 1-(4-(Difluoromethyl)Pyridin-2-yl)-N-(6-Methoxy-1-Methyl-1H-Pyrazolo[4,3-C]Pyridin-7-yl)-1H-Pyrazole-4-Sulfonamide (I-126)

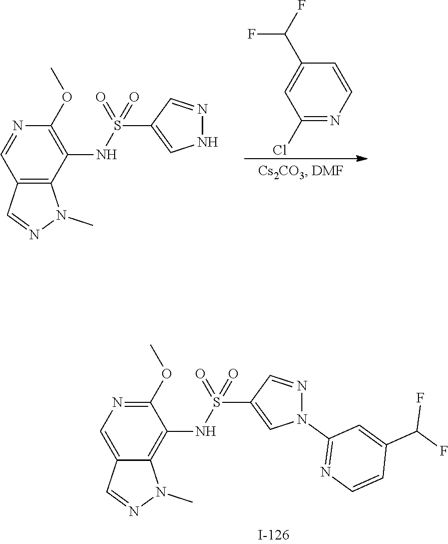 [SMILES: COc1ncc2cnn(C)c2c1NS(=O)(=O)c1cn[nH]c1 ; FC(F)c1ccnc(Cl)c1 ; COc1ncc2cnn(C)c2c1NS(=O)(=O)c1cnn(-c2cc(C(F)F)ccn2)c1]

A solution of N-{6-methoxy-1-methylpyrazolo[4,3-c]pyridin-7-yl}-1H-pyrazole-4-sulfonamide (100 mg, 0.324 mmol, 1 equiv), 2-chloro-4-(difluoromethyl)pyridine (79.57 mg, 0.486 mmol, 1.5 equiv) and Cs₂CO₃ (317.03 mg, 0.972 mmol, 3 equiv) in DMF (1.5 mL) was stirred for 16 h at 80° C. under a nitrogen atmosphere. The reaction was diluted with water (10 mL). The resulting mixture was extracted with EtOAc (3×15 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 1-[4-(difluoromethyl)pyridin-2-yl]-N-{6-methoxy-1-methylpyrazolo[4,3-c]pyridin-7-yl}pyrazole-4-sulfonamide (I-126, 28.7 mg, 20.16%) as a white solid. LCMS (ES, m/z): [M+H]⁺=436; ¹H NMR (400 MHz, DMSO): δ 3.42 (s, 3H), 4.22 (s, 3H), 7.25 (t, J=14.8 Hz, 1H), 7.67 (d, J=5.2 Hz, 1H), 8.08 (s, 1H), 8.13 (s, 1H), 8.23 (s, 1H), 8.67 (s, 1H), 8.71 (d, J=4.8 Hz, 1H), 8.79 (s, 1H), 9.97 (s, 1H).

### Example 419—Synthesis of N-(1-Methyl-1H-Indazol-7-yl)-1-(4-(Piperidin-1-ylmethyl)Pyridin-2-yl)-1H-Pyrazole-4-Sulfonamide (I-486)

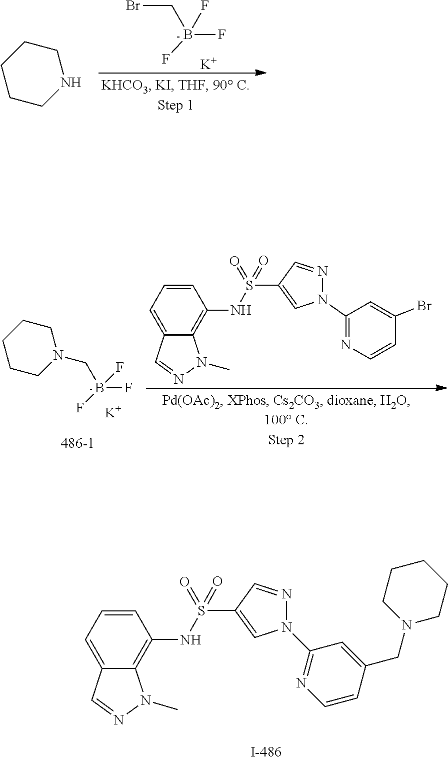

Step 1: Synthesis of 486-1: A solution of piperidine (1 g, 11.744 mmol, 1 equiv), potassium (bromomethyl)trifluoroboranuide (2.36 g, 11.744 mmol, 1 equiv), KHCO₃ (2.35 g, 23.483 mmol, 2 equiv), and KI (1.95 g, 11.766 mmol, 1 equiv) in THF (35 mL) was stirred overnight at 90° C. under a nitrogen atmosphere. The resulting mixture was concentrated under reduced pressure. The residue was dissolved in acetone (50 mL) and refluxed for 3 hours under a nitrogen atmosphere. The resulting mixture was filtered, and the filter cake was washed with acetone (3×5 mL). The filtrate was concentrated under reduced pressure to afford 486-1(1.2 g, crude) as a brown oil. The crude product mixture was used directly in the next step without purification.

Step 2: Synthesis of N-(1-methyl-1H-indazol-7-yl)-1-(4-(piperidin-1-ylmethyl) pyridin-2-yl)-1H-pyrazole-4-sulfonamide (I-486): A solution of 1-(4-bromopyridin-2-yl)-N-(1-methylindazol-7-yl)pyrazole-4-sulfonamide (150 mg, 0.346 mmol, 1 equiv), 486-1 (709.95 mg, 3.460 mmol, 10 equiv), Pd(OAc)₂ (15.54 mg, 0.069 mmol, 0.2 equiv), XPhos (33.01 mg, 0.069 mmol, 0.2 equiv), and Cs₂CO₃ (338.39 mg, 1.038 mmol, 3 equiv) in dioxane (4 mL)/H₂O (1 mL) was stirred for 2 hours at 100° C. under a nitrogen atmosphere. The mixture was allowed to cool to room temperature. The resulting mixture was diluted with water (20 mL). The resulting mixture was extracted with DCM (2×15 mL). The combined organic layers were concentrated under reduced pressure. The crude product was purified by Prep-HPLC with the following conditions (Column: XBridge Shield RP18 OBD Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient: 20% B to 42% B in 9 min; Wave Length: 254 nm/220 nm; RT1(min): 8.8; Number of Runs: 6) to afford N-(1-methyl-1H-indazol-7-yl)-1-(4-(piperidin-1-ylmethyl)pyridin-2-yl)-1H-pyrazole-4-sulfonamide (I-486, 27.6 mg, 17.28%) as an off-white solid. LC-MS (ES, m/z): [M+H]⁺=452. ¹H-NMR: (DMSO-d6, 400 MHz, ppm): δ 1.41 (s, 2H), 1.51-1.55 (m, 4H), 2.38 (s, 4H), 3.59 (s, 2H), 4.29 (s, 3H), 6.70-6.74 (m, 1H), 6.96 (t, J=7.7 Hz, 1H), 7.37-7.42 (m, 1H), 7.65 (s, 1H), 7.94 (s, 1H), 8.01 (d, J=0.8 Hz, 1H), 8.07 (s, 1H), 8.43 (d, J=5.1 Hz, 1H), 8.77 (s, 1H), 10.02 (s, 1H).

### Example 420—Synthesis of 1-(4-((4-Fluoropiperidin-1-yl)Methyl) Pyridin-2-yl)-N-(1-Methyl-1H-Indazol-7-yl)-1H-Pyrazole-4-Sulfonamide (I-487)

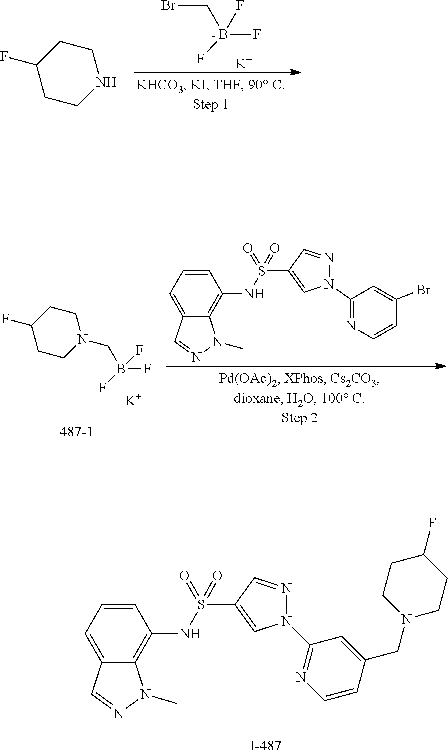

Step 1: Synthesis of 487-1: A solution of 4-fluoropiperidine (500 mg, 4.848 mmol, 1 equiv), potassium (bromomethyl)trifluoroboranuide (973.58 mg, 4.848 mmol, 1.00 equiv), KHCO₃ (970.66 mg, 9.696 mmol, 2.00 equiv), and KI (804.75 mg, 4.848 mmol, 1.00 equiv) in THF (50 mL) was stirred overnight at 90° C. under a nitrogen atmosphere. The resulting mixture was concentrated under reduced pressure. T he residue was dissolved in acetone (50 mL) and refluxed for 3 hours under a nitrogen atmosphere. The resulting mixture was filtered, and the filter cake was washed with acetone (3×5 mL). The filtrate was concentrated under reduced pressure to afford 487-1 (2 g, crude) as a brown oil. The crude product was used directly in the next step without purification.

Step 2: Synthesis of 1-(4-((4-fluoropiperidin-1-yl)methyl)pyridin-2-yl)-N-(1-methyl-1H-indazol-7-yl)-1H-pyrazole-4-sulfonamide (I-487): A mixture of 487-1 (515 mg, 2.310 mmol, 5.00 equiv), 1-(4-bromopyridin-2-yl)-N-(1-methylindazol-7-yl)pyrazole-4-sulfonamide (200 mg, 0.462 mmol, 1.00 equiv), Pd(OAc)₂ (31 mg, 0.138 mmol, 0.30 equiv), XPhos (132 mg, 0.277 mmol, 0.60 equiv), and Cs₂CO₃ (451 mg, 1.384 mmol, 3.00 equiv) in dioxane (10 mL) and H₂O (2 mL) was stirred for 3 hours at 100° C. under a nitrogen atmosphere. The resulting mixture was concentrated under reduced pressure. The resulting mixture was diluted with water (5 mL). The resulting mixture was extracted with CH₂Cl₂ (5×10 mL). The combined organic layers were concentrated under reduced pressure. The crude product (40 mg) was purified by Prep-HPLC with the following conditions (Column: XBridge Prep OBD C18 Column, 30*150 mm, 5 μm; Mobile Phase A: Water(10 mmol/L NH₄HCO₃)+0.05% NH₃·H₂O, Mobile Phase B: ACN; Flow rate: 60 mL/min; Gradient: 13% B to 40% B in 7 min; Wave Length: 254 nm/220 nm; RT1(min): 8.28) to afford 1-(4-((4-fluoropiperidin-1-yl)methyl)pyridin-2-yl)-N-(1-methyl-1H-indazol-7-yl)-1H-pyrazole-4-sulfonamide (I-487, 19 mg, 8.49%) as a white solid. LC-MS: (ES, m/z): [M+H]⁺ 470. ¹H-NMR: (400 MHz, DMSO-d₆, ppm) δ 1.71-1.79 (m, 2H), 1.83-1.94 (m, 2H), 2.34-2.40 (m, 2H), 2.53-2.57 (m, 2H), 3.64 (s, 2H), 4.29 (s, 3H), 4.64-4.80 (m, 1H), 6.72 (dd, J=7.2, 1.2 Hz, 1H), 6.97 (t, J=7.6 Hz, 1H), 7.41 (dd, J=5.2, 1.6 Hz, 1H), 7.67 (d, J=8.0 Hz, 1H), 7.95 (s, 1H), 8.01 (s, 1H), 8.08 (s, 1H), 8.44 (d, J=5.2 Hz, 1H), 8.78 (s, 1H), 10.03 (s, 1H).

### Example 421—Synthesis of N-(1-Methyl-1H-Indazol-7-yl)-1-(4-((4-Methylpiperidin-1-yl)Methyl)Pyridin-2-yl)-1H-Pyrazole-4-Sulfonamide (I-488)

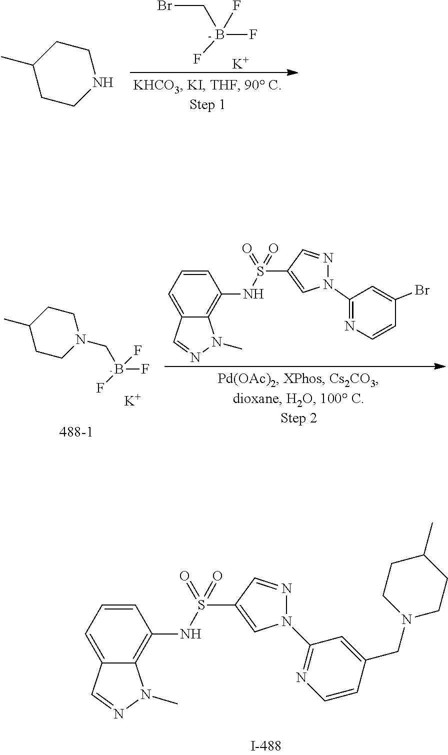

Step 1: Synthesis of 488-1: A solution of 4-methylpiperidine (1 g, 10.083 mmol, 1 equiv), potassium (bromomethyl) trifluoroboranuide (2.25 g, 10.083 mmol, 1 equiv), KHCO₃ (2.02 g, 20.166 mmol, 2 equiv), and KI (1.78 g, 10.083 mmol, 1 equiv) in THF (50 mL) was stirred overnight at 90° C. in an oil bath. The resulting mixture was concentrated under reduced pressure. The residue was dissolved in acetone (50 mL) and refluxed for 3 hours under a nitrogen atmosphere. The resulting mixture was filtered, and the filter cake was washed with acetone (3×5 mL). The filtrate was concentrated under reduced pressure to afford 488-1 (1.2 g, crude) as a brown oil. The crude product mixture was used directly in the next step without purification.

Step 2: Synthesis of N-(1-methyl-1H-indazol-7-yl)-1-(4-((4-methylpiperidin-1-yl)methyl)pyridin-2-yl)-1H-pyrazole-4-sulfonamide (I-488): A solution of 1-(4-bromopyridin-2-yl)-N-(1-methylindazol-7-yl)pyrazole-4-sulfonamide (150 mg, 0.346 mmol, 1 equiv), 488-1 (379.26 mg, 1.730 mmol, 5 equiv), Pd(OAc)₂ (15.54 mg, 0.069 mmol, 0.2 equiv), XPhos (33.01 mg, 0.069 mmol, 0.2 equiv), and Cs₂CO₃ (338.39 mg, 1.038 mmol, 3 equiv) in dioxane (4 mL)/H₂O (1 mL) was stirred for 2 hours at 100° C. under a nitrogen atmosphere. The mixture was allowed to cool to room temperature. The resulting mixture was diluted with water (20 mL). The resulting mixture was extracted with DCM (2×15 mL). The combined organic layers were concentrated under reduced pressure. The crude product was purified by Prep-HPLC with the following conditions (Column: XBridge Shield RP18 OBD Column, 30*150 mm, 5 μm; Mobile Phase A: Water(10 mmol/L NH₄HCO₃+0.05% NH₃H₂O), Mobile Phase B: ACN; Flow rate: 60 mL/min; Gradient: 25% B to 55% B in 7 min; Wave Length: 254 nm/220 nm; RT1(min): 7.2; Number Of Runs: 2) to afford N-(1-methyl-1H-indazol-7-yl)-1-(4-((4-methylpiperidin-1-yl)methyl)pyridin-2-yl)-1H-pyrazole-4-sulfonamide (I-488, 21.2 mg, 12.89%) as an off-white solid. LC-MS (ES, m/z): [M+H]⁺ 466. ¹H-NMR: (DMSO-d6, 400 MHz, ppm): δ 0.90 (d, J=6.4 Hz, 3H), 1.14-1.23 (m, 2H), 1.35 (s, 1H), 1.59 (d, J=12.4 Hz, 3H), 2.00 (t, J=11.4 Hz, 3H), 2.78 (d, J=11.2 Hz, 2H), 3.60 (s, 2H), 4.29 (s, 3H), 6.69-6.75 (m, 1H), 6.96 (t, J=7.8 Hz, 1H), 7.39 (d, J=5.0 Hz, 1H), 7.64 (s, 1H), 7.94 (s, 1H), 8.01 (s, 1H), 8.07 (s, 1H), 8.43 (d, J=5.0 Hz, 1H), 8.77 (s, 1H), 10.02 (s, 1H).

### Example 422—Synthesis of N-(6-Hydroxy-1-Methyl-1H-Indazol-7-yl)-1-(4-(Trifluoromethyl)Pyridin-2-yl)-1H-Pyrazole-4-Sulfonamide (I-489)

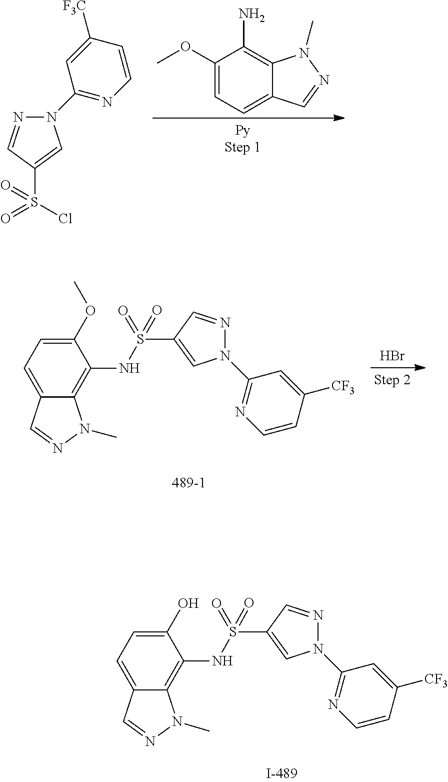

Step 1: Synthesis of 489-1: To a stirred mixture of 1-[4-(trifluoromethyl)pyridin-2-yl]pyrazole-4-sulfonyl chloride (300 mg, 0.963 mmol, 1 equiv) in pyridine (10 mL) was added 6-methoxy-1-methylindazol-7-amine (255.87 mg, 1.444 mmol, 1.5 equiv) in portions at room temperature under air atmosphere. The resulting mixture was stirred for 1 hour at room temperature. The mixture was concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This afforded 489-1 (311 mg, 71.41%) as a yellow solid.

Step 2: Synthesis of N-(6-hydroxy-1-methyl-1H-indazol-7-yl)-1-(4-(trifluoromethyl)pyridin-2-yl)-1H-pyrazole-4-sulfonamide (I-489): A solution of 489-1 (160 mg) in HBr in AcOH (40%) (20 mL) was stirred for 3 hours at 100° C. The mixture was concentrated under reduced pressure. The residue was purified by reversed-phase flash chromatography with the following conditions (column, C18 silica gel; mobile phase, MeCN in water (0.1% FA), 0% to 50% gradient in 20 min; detector, UV 254 nm) to afford the crude product. The crude product was purified by Prep-HPLC with the following conditions (Column: Xselect CSH C18 OBD Column 30*150 mm 5 μm; Mobile Phase A: Water (0.1% FA), Mobile Phase B: ACN; Flow rate: 60 mL/min; Gradient: 37% B to 50% B in 7 min; Wave Length: 254 nm/220 nm; RT1(min): 6.2) to afford N-(6-hydroxy-1-methyl-1H-indazol-7-yl)-1-(4-(trifluoromethyl)pyridin-2-yl)-1H-pyrazole-4-sulfonamide (I-489, 1.4 mg, 2.79%) as a light-brown solid. LC-MS: (ES, m/z): [M+1]=439. ¹H-NMR: (400 MHz, DMSO-d₆) δ 4.21 (s, 3H), 6.63 (d, J=8.6 Hz, 1H), 7.45 (d, J=8.6 Hz, 1H), 7.85 (d, J=1.6 Hz, 2H), 7.86 (s, 1H), 8.04 (s, 1H), 8.77-8.84 (m, 2H), 9.66 (s, 2H).

### Example 423—Synthesis of 1-(4-(5-Hydroxyspiro[2.3]Hexan-5-yl)Pyridin-2-yl)-N-(1-Methyl-1H-Indazol-7-yl)-1H-Pyrazole-4-Sulfonamide (I-490)

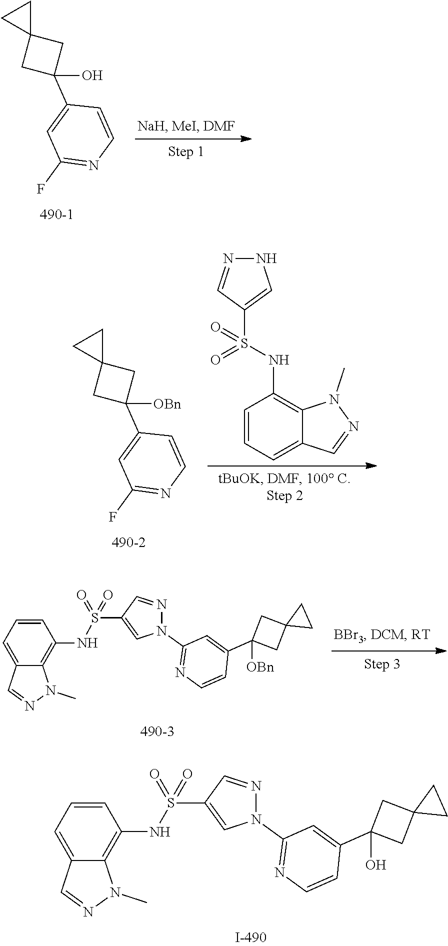 [SMILES: OC1(c2ccnc(F)c2)CC2(CC2)C1 ; Fc1cc(C2(OCc3ccccc3)CC3(CC3)C2)ccn1 ; Cn1ncc2cccc(NS(=O)(=O)c3cn[nH]c3)c21 ; Cn1ncc2cccc(NS(=O)(=O)c3cnn(-c4cc(C5(OCc6ccccc6)CC6(CC6)C5)ccn4)c3)c21 ; Cn1ncc2cccc(NS(=O)(=O)c3cnn(-c4cc(C5(O)CC6(CC6)C5)ccn4)c3)c21]

Step 1: Synthesis of 490-1: To a solution of 490-1 (400 mg, 2.070 mmol, 1 equiv) in THE (5 mL) was added NaH (74.52 mg, 3.105 mmol, 1.5 equiv) at 0° C. under a nitrogen atmosphere. The mixture was stirred for 30 minutes followed by the addition of benzyl bromide (0.49 mL, 4.140 mmol, 2.00 equiv). The resulting mixture was stirred for 2 hours at room temperature under a nitrogen atmosphere. The desired product could be detected by LCMS. The reaction was quenched with ice at 0° C. The resulting mixture was extracted with EtOAc (2×200 mL). The combined organic layers were washed with brine (1×150 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. This afforded 490-2 (320 mg, 50.19%) as a light-yellow solid.

Step 2: Synthesis of 490-2: To a solution of 490-2 (150 mg, 0.529 mmol, 1.00 equiv) in DMF (3 mL) was added N-(1-methylindazol-7-yl)-1H-pyrazole-4-sulfonamide (146.80 mg, 0.529 mmol, 1 equiv) followed by t-BuOK (178.21 mg, 1.587 mmol, 3 equiv). The resulting mixture was stirred for 2 hours at 100° C. under a nitrogen atmosphere. The desired product could be detected by LCMS. The reaction was quenched with water at room temperature. The resulting mixture was extracted with EtOAc (2×100 mL). The combined organic layers were washed with brine (120 mL) and dried over anhydrous Na₂SO₄. After filtration, the filtrate was concentrated under reduced pressure. The residue was purified by Prep-TLC (PE/EA=1:1) to afford 490-3 (120 mg, 38.99%) as a light-yellow solid.

Step 3: Synthesis of 1-(4-(5-hydroxyspiro[2.3]hexan-5-yl)pyridin-2-yl)-N-(1-methyl-1H-indazol-7-yl)-1H-pyrazole-4-sulfonamide (I-490): To a solution of 490-3 (100 mg, 0.185 mmol, 1 equiv) in DCM (5 mL) was added BBr₃ (0.09 mL, 0.925 mmol, 5 equiv) at 0° C. The resulting mixture was stirred for 2 hours at room temperature under a nitrogen atmosphere. The reaction was quenched with MeOH at 0° C. The resulting mixture was extracted with DCM (2×100 mL). The combined organic layers were concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. The crude product (40 mg) was purified by Prep-HPLC with the following conditions (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient: 17% B to 42% B in 8 min; Wave Length: 220 nm; RT1(min): 7.63) to afford 1-(4-(5-hydroxyspiro[2.3]hexan-5-yl)pyridin-2-yl)-N-(1-methyl-1H-indazol-7-yl)-1H-pyrazole-4-sulfonamide (I-490, 4.4 mg, 5.04%) as a white solid. LC-MS: (ES, m/z): [M+H]⁺=451. ¹H NMR: (400 MHz, DMSO, ppm) δ 0.61 (q, J=3.3 Hz, 4H), 2.33-2.40 (m, 2H), 2.67 (d, J=13.2 Hz, 2H), 4.29 (s, 3H), 6.20 (s, 1H), 6.72 (d, J=7.2 Hz, 1H), 6.97 (t, J=7.8 Hz, 1H), 7.67 (m, J=5.2, 1.6 Hz, 2H), 8.02 (d, J=3.2 Hz, 1H), 8.08 (d, J=6.6 Hz, 1H), 8.21 (t, J=1.2 Hz, 1H), 8.50 (d, J=5.2 Hz, 1H), 8.80 (s, 1H), 10.03 (s, 1H).

### Example 424—Synthesis of 1-(4-(5-Hydroxyspiro[2.3]Hexan-5-yl)Pyridin-2-yl)-N-(6-Methoxy-1-Methyl-1H-Indazol-7-yl)-1H-Pyrazole-4-Sulfonamide (I-491)

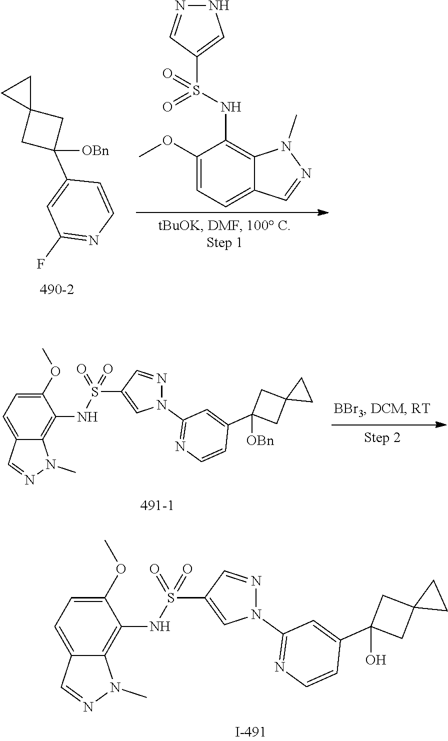 [SMILES: Fc1cc(C2(OCc3ccccc3)CC3(CC3)C2)ccn1 ; COc1ccc2cnn(C)c2c1NS(=O)(=O)c1cn[nH]c1 ; COc1ccc2cnn(C)c2c1NS(=O)(=O)c1cnn(-c2cc(C3(OCc4ccccc4)CC4(CC4)C3)ccn2)c1 ; COc1ccc2cnn(C)c2c1NS(=O)(=O)c1cnn(-c2cc(C3(O)CC4(CC4)C3)ccn2)c1]

Step 1: Synthesis of 491-1: To a solution of 490-2 (150 mg, 0.529 mmol, 1 equiv) in DMF (3 mL) was added N-(6-methoxy-1-methylindazol-7-yl)-1H-pyrazole-4-sulfonamide (146.43 mg, 0.476 mmol, 0.9 equiv) followed by t-BuOK (5.94 mg, 0.054 mmol, 3 equiv). The resulting mixture was stirred for 2 hours at 100° C. under a nitrogen atmosphere. The desired product could be detected by LCMS. The reaction was quenched with water at room temperature. The resulting mixture was extracted with EtOAc (2×100 mL). The combined organic layers were washed with brine (50 mL) and dried over anhydrous Na₂SO₄. After filtration, the filtrate was concentrated under reduced pressure. The residue was purified by Prep-TLC (PE/EA=1:1) to afford 491-1 (100 mg, 28.14%) as a light-yellow solid.

Step 2: Synthesis of 1-(4-(5-hydroxyspiro[2.3]hexan-5-yl)pyridin-2-yl)-N-(6-methoxy-1-methyl-1H-indazol-7-yl)-1H-pyrazole-4-sulfonamide (I-491): To a solution of 491-1 (80 mg, 0.140 mmol, 1 equiv) in DCM (5 mL) was added BBr₃ (0.07 mL, 0.700 mmol, 5 equiv) at 0° C. The resulting mixture was stirred for 2 hours at room temperature under a nitrogen atmosphere. The reaction was quenched with MeOH at 0° C. The resulting mixture was extracted with DCM (2×100 mL). The combined organic layers were washed with brine (1×100 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. The crude product (30 mg) was purified by Prep-HPLC with the following conditions (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient: 17% B to 42% B in 8 min; Wave Length: 220 nm; RT1(min): 7.63) to afford 1-(4-(5-hydroxyspiro[2.3]hexan-5-yl)pyridin-2-yl)-N-(6-methoxy-1-methyl-1H-indazol-7-yl)-1H-pyrazole-4-sulfonamide (I-491, 7.1 mg, 10.51%) as a white solid. LC-MS: (ES, m/z): [M+H]=481. ¹H-NMR: (400 MHz, Chloroform-d) δ 0.61-0.73 (m, 4H), 2.54-2.62 (m, 2H), 2.64-2.71 (m, 2H), 3.43 (s, 3H), 4.45 (s, 3H), 6.50 (s, 1H), 6.71 (d, J=8.8 Hz, 1H), 7.58 (m, J=5.2, 1.6 Hz, 1H), 8.75 (s, 1H), 7.64 (d, J=8.8 Hz, 1H), 7.71 (s, 1H), 7.99 (s, 1H), 8.22 (d, J=1.6 Hz, 1H), 8.40 (d, J=5.2 Hz, 1H).

### Example 425—Synthesis of 1-(4-(3-Methoxycyclobutyl)Pyridin-2-yl)-N-(1-Methyl-1H-Indazol-7-yl)-1H-Pyrazole-4-Sulfonamide (I-492)

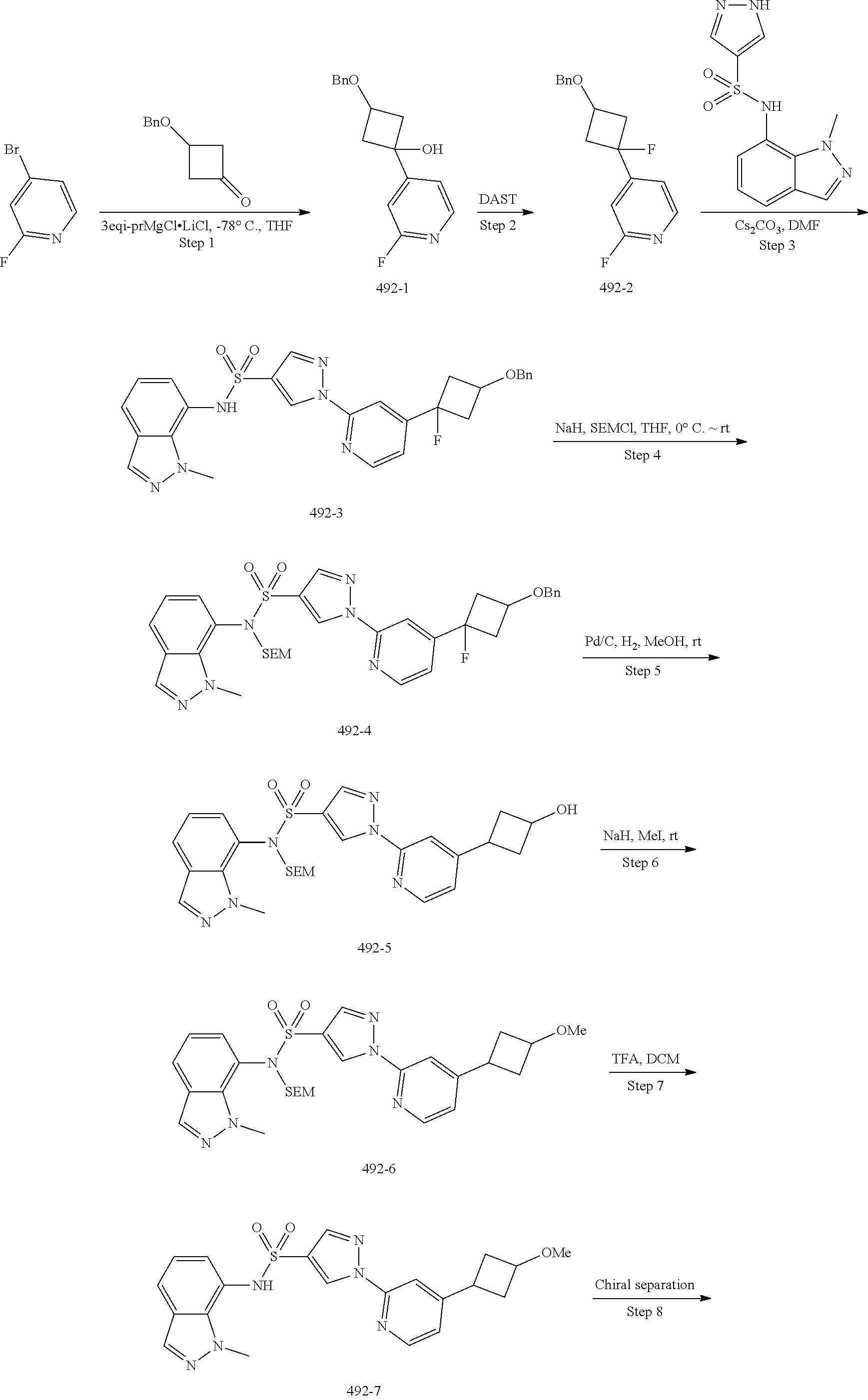

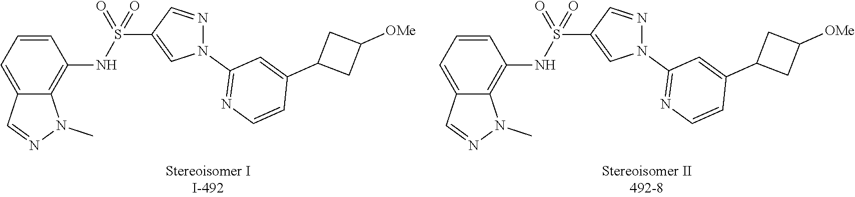 [SMILES: COC1CC(c2ccnc(-n3cc(S(=O)(=O)Nc4cccc5cnn(C)c45)cn3)c2)C1 ; COC1CC(c2ccnc(-n3cc(S(=O)(=O)Nc4cccc5cnn(C)c45)cn3)c2)C1]

Step 1: Synthesis of 492-1: A solution of 4-bromo-2-fluoropyridine (5 g, 28.411 mmol, 1 equiv) in THF (100 mL) was treated with i-PrMgCl—LiCl (1.3 M in THF) (12.38 g, 85.233 mmol, 3 equiv) for 1 hour at −78° C. under a nitrogen atmosphere after which 3-(benzyloxy)cyclobutan-1-one (15.02 g, 85.233 mmol, 3 equiv) in THE (50 mL) was added at −60° C. The resulting mixture was stirred for 2 hours at −60° C. under a nitrogen atmosphere. The reaction was quenched with NH₄Cl (aq.) at 0° C. The resulting mixture was extracted with EtOAc (3×400 mL) and concentrated under reduced pressure. The residue was purified by silica gel column chromatography, eluting with PE/EA (20:1) to afford 492-1 (1.5 g, 19.31%) as a brown-yellow oil.

Step 2: Synthesis of 492-2: To a solution of 492-1 (1.5 g, 5.488 mmol, 1 equiv) in DCM (30 mL) was added DAST (2.2 mL, 16.651 mmol, 3.03 equiv) for 2 minutes at −78° C. The resulting mixture was stirred for 2 hours at −78° C. under a nitrogen atmosphere. The reaction was quenched by the addition of water/ice (150 mL) at 0° C. The resulting mixture was extracted with DCM (3×100 mL) and concentrated under reduced pressure. The residue was purified by reversed-phase flash chromatography with the following conditions: column, C18 silica gel; mobile phase, MeCN in water (10 mmol/L NH₄HCO₃), 0% to 70% gradient in 40 min; detector, UV 220 nm. This afforded 492-2 (780 mg, 51.62%) as a light-yellow oil.

Step 3: Synthesis of 492-3: To a stirred solution of 492-2 (567 mg, 2.059 mmol, 1 equiv) and Cs₂CO₃ (1.99 g, 3.744 mmol, 2.97 equiv) in DMF (10 mL) was added N-(1-methylindazol-7-yl)-1H-pyrazole-4-sulfonamide (571.9 mg, 2.062 mmol, 1.00 equiv) dropwise at room temperature under a nitrogen atmosphere. The resulting mixture was stirred for 5 hours at 80° C. under a nitrogen atmosphere. The resulting mixture was diluted with water. The resulting mixture was extracted with EtOAc (3×100 mL) and concentrated under reduced pressure. The residue was purified by reversed-phase flash chromatography with the following conditions: column, C18 silica gel; mobile phase, MeCN in water (10 mmol/L NH₄HCO₃), 0% to 50% gradient in 20 min; detector, UV 220 nm. This afforded 492-3 (862 mg, 78.59%) as a light-brown solid.

Step 4: Synthesis of 492-4: A solution of 492-3 (842 mg, 1.581 mmol, 1 equiv) in THF (10 mL) was treated with NaH (110 mg, 1.917 mmol, 1.21 equiv, 60%) for 20 minutes at 0° C. under a nitrogen atmosphere followed by the dropwise addition of SEMCl (421 mg, 2.372 mmol, 1.5 equiv) at 0° C. The resulting mixture was stirred for 1 hours at room temperature under a nitrogen atmosphere. The reaction was quenched with NH₄Cl (aq.) (80 mL) at 0° C. The resulting mixture was extracted with EtOAc (3×100 mL) and concentrated under reduced pressure. The residue was purified by reversed-phase flash chromatography with the following conditions: column, C18 silica gel; mobile phase, MeCN in water (10 mmol/L NH₄HCO₃), 90% to 95% gradient in 30 min; detector, UV 220 nm. This afforded 492-4 (868 mg, 82.83%) as a brown oil.

Step 5: Synthesis of 492-5: A solution of 492-4 (616 mg, 0.929 mmol, 1 equiv) and Pd/C (123 mg, 20%) in MeOH (25 mL) was stirred for 3 hours at room temperature under a hydrogen atmosphere. The resulting mixture was filtered, and the filter cake was washed with MeOH (5×10 mL). The filtrate was concentrated under reduced pressure. The residue was purified by reversed-phase flash chromatography with the following conditions: column, C18 silica gel; mobile phase, MeCN in water (10 mmol/L NH₄HCO₃), 60% to 62% gradient in 25 min; detector, UV 220 nm. This afforded 492-5 (200 mg, 38.80%) as a white solid.

Step 6: Synthesis of 492-6: A solution of 492-5 (1.25 mg, 0.360 mmol, 1 equiv) in THE (10 mL) was treated with NaH (17.5 mg, 0.729 mmol, 2.02 equiv, 60%) for 30 minutes at 0° C. under a nitrogen atmosphere followed by Mel (154 mg, 1.084 mmol, 3.00 equiv) dropwise at 0° C. The resulting mixture was stirred for 2 hours at room temperature under a nitrogen atmosphere. The resulting mixture was diluted with water (10 mL). The resulting mixture was extracted with CH₂Cl₂ (4×10 mL). The combined organic layers were dried over anhydrous Na₂SO₄. After filtration, the filtrate was concentrated under reduced pressure. This afforded 492-6 (162 mg, 87.78%) as a white solid.

Step 7: Synthesis of 492-7: A solution of 492-6 (170 mg, 0.299 mmol, 1 equiv) and TFA (10 mL, 134.631 mmol, 450.43 equiv) in DCM (3 mL) was stirred for 4 hours at 50° C. under a nitrogen atmosphere. The resulting mixture was concentrated under reduced pressure. The residue was purified by reversed-phase flash chromatography with the following conditions: column, C18 silica gel; mobile phase, MeCN in Water (10 mmol/L NH₄HCO₃), 45% to 50% gradient in 20 min; detector, UV 220 nm. This afforded 492-7 (110 mg, 83.96%) as a white solid.

Step 8: Synthesis of 1-(4-(3-methoxycyclobutyl)pyridin-2-yl)-N-(1-methyl-1H-indazol-7-yl)-1H-pyrazole-4-sulfonamide (I-492): The crude product (110 mg) was purified by Prep-SFC with the following conditions (Column: CHIRALPAK IG, 3*25 cm, 5 μm; Mobile Phase A: CO₂, Mobile Phase B: MeOH; Flow rate: 100 mL/min; Gradient: isocratic 48% B; Back Pressure(bar): 100; Wave Length: 220 nm; RT1(min): 16.22; RT2(min): 19.08; Sample Solvent: MeOH; Injection Volume: 2.5 mL) to afford 1-(4-((1s,3s)-3-methoxycyclobutyl)pyridin-2-yl)-N-(1-methyl-1H-indazol-7-yl)-1H-pyrazole-4-sulfonamide (I-492, 69.7 mg, 52.22%) as an off-white solid and the crude of 492-8 (10 mg). The crude product (10 mg) was purified by Prep-Chiral-HPLC with the following conditions (Column: CHIRALPAK IG, 2*25 cm, 5 μm; Mobile Phase A: Hex(0.2% FA), Mobile Phase B: EtOH: MeOH=1:1-HPLC; Flow rate: 20 mL/min; Gradient: 50% B to 50% B in 28 min; Wave Length: 220/254 nm; RT1(min): 20.6; RT2(min): 24.06; Sample Solvent: EtOH:DCM=1:1-HPLC; Injection Volume: 0.4 mL; Number of Runs: 3) to afford 492-8 as a single stereoisomer (4.6 mg, 3.49%) as an off-white solid. I-492: LC-MS: (ES, m/z): [M+H]⁺ 439. ¹H-NMR: (400 MHz, CD₃OD, ppm) δ 2.58-2.40 (m, 4H), 3.29 (s, 3H), 3.65-3.73 (m, 1H), 4.09-4.16 (m, 1H), 4.37 (s, 3H), 6.74 (d, J=8.0 Hz 1H), 6.95 (t, J=7.6 Hz, 1H), 7.29 (dd, J=5.2, 1.6 Hz, 1H), 7.68 (d, J=8.0 Hz, 1H), 7.85 (s, 1H), 7.90 (d, J=1.6 Hz, 1H), 8.02 (s, 1H), 8.33 (d, J=5.2 Hz, 1H), 8.77 (s, 1H). 492-8: LC-MS: (ES, m/z): [M+H]⁺ 439. ¹H-NMR: (400 MHz, CD₃OD, ppm) δ 2.00-2.08 (m, 2H), 2.77-2.83 (m, 2H), 3.19-3.23 (m, 1H), 3.93-4.03 (m, 1H), 3.30 (s, 3H), 4.31 (s, 3H), 6.73 (dd, J=7.2, 1.2 Hz, 1H), 6.95-7.01 (m, 1H), 7.29 (d, J=5.1 Hz, 1H), 7.72 (dd, J=8.4, 1.2 Hz, 1H), 7.85 (s, 1H), 7.90 (s, 1H), 8.03 (s, 1H), 8.35 (d, J=5.0 Hz, 1H), 8.77 (s, 1H).

### Example 426—Synthesis of (R)—N-(1-Methyl-1H-Indazol-7-yl)-1-(4-(1-(Pyrrolidin-1-yl)Ethyl)Pyridin-2-yl)-1H-Pyrazole-4-Sulfonamide (I-494)

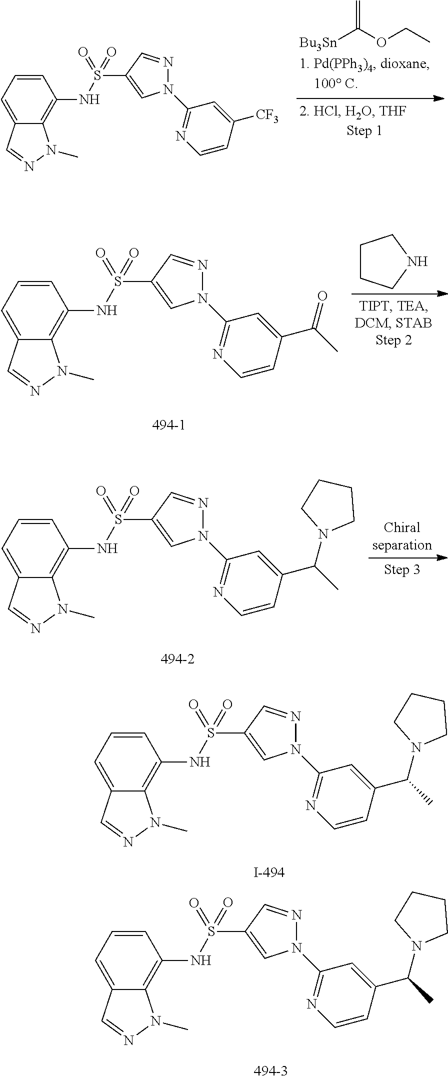

Step 1: Synthesis of 494-1: A solution of 1-(4-bromopyridin-2-yl)-N-(1-methylindazol-7-yl)pyrazole-4-sulfonamide (1 g, 2.308 mmol, 1 equiv), tributyl(1-ethoxyethenyl)stannane (1.25 g, 3.462 mmol, 1.5 equiv), and Pd(PPh₃)₄(0.27 g, 0.231 mmol, 0.1 equiv) in dioxane (20 mL) was stirred for 2 hours at 100° C. under a nitrogen atmosphere. The mixture was allowed to cool to room temperature. To the above mixture was added 1M HCl (3 mL) dropwise at room temperature. The resulting mixture was stirred for 1 hour at room temperature. The resulting mixture was diluted with water (100 mL). The resulting mixture was extracted with EtOAc (3×50 mL). The combined organic layers were concentrated under reduced pressure. The residue was purified by reversed-phase flash chromatography with the following conditions: column, C18 silica gel; mobile phase, MeCN in water (0.1% TFA), 30% to 50% gradient in 10 min; detector, UV 254 nm. This resulted in 494-1 (650 mg, 57.54%) as a grey solid.

Step 2: Synthesis of 494-2: To a stirred solution of 494-1 (400 mg, 1.009 mmol, 1 equiv) and pyrrolidine (215.28 mg, 3.027 mmol, 3 equiv) in DCM (20 mL) was added TEA (510.52 mg, 5.045 mmol, 5 equiv) and Ti(Oi-Pr)₄ (860.34 mg, 3.027 mmol, 3 equiv) at room temperature. The resulting mixture was stirred for 2 hours at room temperature. To the above mixture was added STAB (641.55 mg, 3.027 mmol, 3 equiv) at room temperature. The resulting mixture was stirred for overnight at room temperature. The reaction was quenched with water at room temperature. The aqueous layer was extracted with DCM (3×100 mL). The residue was purified by reversed-phase flash chromatography with the following conditions: column, C18 silica gel; mobile phase, MeCN in water (10 mmol/L NH₄HCO₃), 0% to 50% gradient in 20 min; detector, UV 254 nm. This afforded 494-2 (70 mg, 15.22%) as an off-white solid.

Step 3: Synthesis of (R)—N-(1-methyl-1H-indazol-7-yl)-1-(4-(1-(pyrrolidin-1-yl)ethyl)pyridin-2-yl)-1H-pyrazole-4-sulfonamide I-494: The 494-2 (70 mg) was purified by Prep-Chiral-HPLC with the following conditions (Column: CHIRALPAK IG, 2*25 cm, 5 μm; Mobile Phase A: MTBE(0.5% 2M NH₃-MeOH), Mobile Phase B: EtOH:HEX=1:6-HPLC; Flow rate: 20 mL/min; Gradient: 30% B to 30% B in 10 min; Wave Length: 220/254 nm; RT1(min): 5.36; RT2(min): 6.66; Sample Solvent: EtOH:DCM=1:1-HPLC; Injection Volume: 0.7 mL; Number of Runs: 3) to afford (R)—N-(1-methyl-1H-indazol-7-yl)-1-(4-(1-(pyrrolidin-1-yl)ethyl)pyridin-2-yl)-1H-pyrazole-4-sulfonamide (I-494, 6.5 mg, 9.12%) and 494-3 (5.2 mg, 7.15%) as an off-white solid. I-494: LC-MS: (ES, m/z): [M+H]⁺=452. ¹H-NMR: (400 MHz, Methanol-d₄) δ 1.45 (d, J=6.6 Hz, 3H), 1.82 (s, 4H), 2.43-2.51 (m, 2H), 2.65 (d, J=6.2 Hz, 2H), 3.42 (q, J=6.8 Hz, 1H), 4.38 (s, 3H), 6.75 (dd, J=7.4, 1.0 Hz, 1H), 6.96 (dd, J=8.0, 7.4 Hz, 1H), 7.39 (dd, J=5.2, 1.6 Hz, 1H), 7.66 (d, J=8.2 Hz, 1H), 7.84 (d, J=0.8 Hz, 1H), 8.01 (s, 1H), 8.04 (d, J=1.2 Hz, 1H), 8.38-8.40 (m, 1H), 8.77 (d, J=0.8 Hz, 1H). 494-3: LC-MS: (ES, m/z): [M+H]⁺=452. ¹H-NMR: (400 MHz, Methanol-d₄) δ 1.45 (d, J=6.6 Hz, 3H), 1.82 (s, 4H), 2.43-2.51 (m, 2H), 2.65 (d, J=6.2 Hz, 2H), 3.42 (q, J=6.8 Hz, 1H), 4.38 (s, 3H), 6.75 (dd, J=7.4, 1.0 Hz, 1H), 6.96 (dd, J=8.0, 7.4 Hz, 1H), 7.39 (dd, J=5.2, 1.6 Hz, 1H), 7.66 (d, J=8.2 Hz, 1H), 7.84 (d, J=0.8 Hz, 1H), 8.01 (s, 1H), 8.04 (d, J=1.2 Hz, 1H), 8.38-8.40 (m, 1H), 8.77 (d, J=0.8 Hz, 1H).

### Example 427—Synthesis of (R)-1-(4-(1-(Azetidin-1-yl)Ethyl)Pyridin-2-yl)-N-(1-Methyl-1H-Indazol-7-yl)-1H-Pyrazole-4-Sulfonamide (I-496)

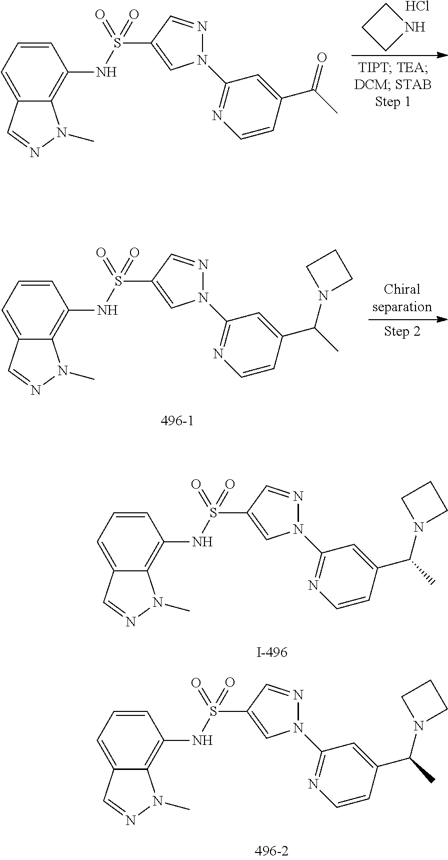

Step 1: Synthesis of 496-1: To a stirred solution of 1-(4-acetylpyridin-2-yl)-N-(1-methyl-1H-indazol-7-yl)-1H-pyrazole-4-sulfonamide (300 mg, 0.757 mmol, 1 equiv) and azetidine hydrochloride (212.39 mg, 2.271 mmol, 3 equiv) in DCM (20 mL) was added TEA (382.89 mg, 3.785 mmol, 5 equiv) and Ti(Oi-Pr)₄ (645.25 mg, 2.271 mmol, 3 equiv) at room temperature. The resulting mixture was stirred for 2 hours at room temperature. To the above mixture was added STAB (481.16 mg, 2.271 mmol, 3 equiv) at room temperature. The resulting mixture was stirred for overnight at room temperature. The reaction was quenched with water at room temperature. The aqueous layer was extracted with DCM (3×100 mL). The residue was purified by reversed-phase flash chromatography with the following conditions: column, C18 silica gel; mobile phase, MeCN in water (10 mmol/L NH₄HCO₃), 0% to 50% gradient in 20 min; detector, UV 254 nm. This afforded 496-1 (160 mg, 46.39%) as an off-white solid.

Step 2: Synthesis of (R)-1-(4-(1-(azetidin-1-yl)ethyl)pyridin-2-yl)-N-(1-methyl-1H-indazol-7-yl)-1H-pyrazole-4-sulfonamide (I-496): The 496-1 (160 mg) was purified by Prep-Chiral-HPLC with the following conditions (Column: CHIRALPAK IG, 2*25 cm, 5 μm; Mobile Phase A: MTBE (0.5% 2M NH₃-MeOH), Mobile Phase B: EtOH:HEX=1:6-HPLC; Flow rate: 20 mL/min; Gradient: 30% B to 30% B in 20 min; Wave Length: 220/254 nm; RT1(min): 13.9; RT2(min): 15.27; Sample Solvent: EtOH:DCM=1:1-HPLC; Injection Volume: 0.6 mL; Number of Runs: 7) to afford I-496 (11.1 mg) as a white solid and 496-2 (11.9 mg) as a white solid. I-496: LC-MS: (ES, m/z): [M+H]⁺=438. ¹H-NMR: (400 MHz, Methanol-d₄) δ 1.26 (d, J=6.6 Hz, 3H), 2.10 (p, J=7.2 Hz, 2H), 3.19-3.27 (m, 4H), 3.56 (q, J=6.6 Hz, 1H), 4.37 (s, 3H), 6.73 (dd, J=7.2, 1.0 Hz, 1H), 6.97 (dd, J=8.2, 7.2 Hz, 1H), 7.35 (dd, J=5.2, 1.6 Hz, 1H), 7.70 (dd, J=8.2, 1.0 Hz, 1H), 7.85 (d, J=0.8 Hz, 1H), 7.99-8.05 (m, 2H), 8.38 (dd, J=5.2, 0.8 Hz, 1H), 8.77 (d, J=0.8 Hz, 1H). 496-2: LC-MS: (ES, m/z): [M+H]⁺=438. ¹H-NMR: (400 MHz, Methanol-d4) δ 1.26 (d, J=6.6 Hz, 3H), 2.10 (p, J=7.2 Hz, 2H), 3.19-3.27 (m, 4H), 3.56 (q, J=6.6 Hz, 1H), 4.37 (s, 3H), 6.73 (dd, J=7.2, 1.0 Hz, 1H), 6.97 (dd, J=8.2, 7.2 Hz, 1H), 7.35 (dd, J=5.2, 1.6 Hz, 1H), 7.70 (dd, J=8.2, 1.0 Hz, 1H), 7.85 (d, J=0.8 Hz, 1H), 7.99-8.05 (m, 2H), 8.38 (dd, J=5.2, 0.8 Hz, 1H), 8.77 (d, J=0.8 Hz, 1H).

### Example 428—Synthesis of (R)—N-(1-Methyl-1H-Indazol-7-yl)-1-(4-(1-(Methylamino)Ethyl)Pyridin-2-yl)-1H-Pyrazole-4-Sulfonamide (I-498)

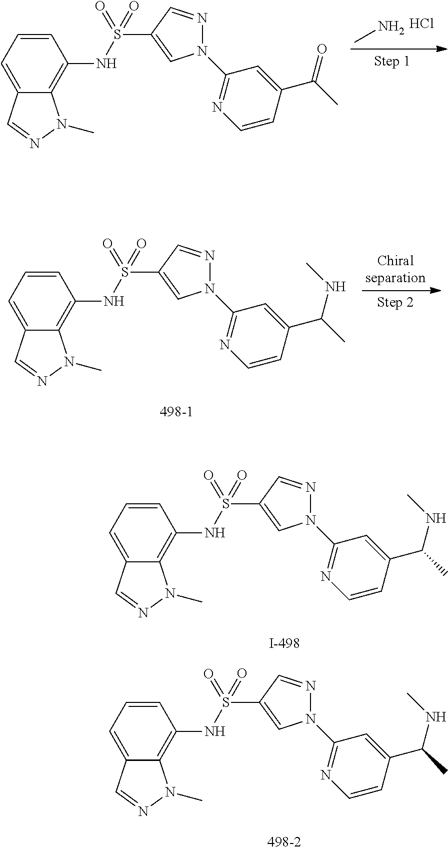

Step 1: Synthesis of 498-1: A solution of 1-(4-acetylpyridin-2-yl)-N-(1-methylindazol-7-yl)pyrazole-4-sulfonamide (300 mg, 0.757 mmol, 1 equiv), methanamine hydrochloride (255.47 mg, 3.785 mmol, 5 equiv), TEA (765.78 mg, 7.570 mmol, 10 equiv), and tetrakis(propan-2-yloxy)titanium (860.32 mg, 3.028 mmol, 4 equiv) in THE (6 mL) was stirred for 3 hours at 60° C. To the above mixture was added NaBH₃CN (95.11 mg, 1.514 mmol, 2 equiv) at room temperature. The resulting mixture was stirred for 1 hour at room temperature. The resulting mixture was diluted with water (30 mL). The resulting mixture was extracted with DCM (3×30 mL). The combined organic layers were concentrated under reduced pressure. The residue was purified by reversed-phase flash chromatography with the following conditions: column, C18 silica gel; mobile phase, MeCN in water (0.1% TFA), 20% to 35% gradient in 10 min; detector, UV 254 nm. This afforded 498-1 (100 mg, 29.55%) as a white solid.

Step 2: Synthesis of (R)—N-(1-methyl-1H-indazol-7-yl)-1-(4-(1-(methylamino)ethyl)pyridin-2-yl)-1H-pyrazole-4-sulfonamide (I-498): The 498-1 (100 mg) was purified by Prep-Chiral-HPLC with the following conditions (Column: CHIRALPAK SZ 2*25 cm, 5 μm; Mobile Phase A: Hex(0.5% 2M NH₃-MeOH), Mobile Phase B: EtOH:DCM=1:1-HPLC; Flow rate: 20 mL/min; Gradient: 20% B to 20% B in 20 min; Wave Length: 220/254 nm; RT1(min): 14.99; RT2(min): 16.27; Sample Solvent: EtOH:DCM=1:1-HPLC; Injection Volume: 0.24 mL; Number Of Runs: 15) to afford (R)—N-(1-methyl-1H-indazol-7-yl)-1-(4-(1-(methylamino)ethyl)pyridin-2-yl)-1H-pyrazole-4-sulfonamide (I-498, 25.3 mg, 25.27%) and 498-2 (26.1 mg, 25.63%) as an off-white solid. I-498: LC-MS: (ES, m/z): [M+H]⁺=412. ¹H-NMR: (400 MHz, Methanol-d₄) δ 1.42 (d, J=6.6 Hz, 3H), 2.30 (s, 3H), 3.84 (t, J=6.8 Hz, 1H), 4.38 (s, 3H), 6.74 (dd, J=7.4, 1.0 Hz, 1H), 6.94-6.99 (m, 1H), 7.38 (dd, J=5.2, 1.6 Hz, 1H), 7.69 (dd, J=8.0, 1.0 Hz, 1H), 7.85 (d, J=0.8 Hz, 1H), 8.03 (d, J=2.8 Hz, 2H), 8.41 (d, J=5.2 Hz, 1H), 8.78 (d, J=0.6 Hz, 1H). 498-2: LC-MS: (ES, m/z): [M+H]⁺=412. ¹H-NMR: (400 MHz, Methanol-d₄) δ 1.42 (d, J=6.6 Hz, 3H), 2.30 (s, 3H), 3.84 (t, J=6.8 Hz, 1H), 4.38 (s, 3H), 6.74 (dd, J=7.4, 1.0 Hz, 1H), 6.94-6.99 (m, 1H), 7.38 (dd, J=5.2, 1.6 Hz, 1H), 7.69 (dd, J=8.0, 1.0 Hz, 1H), 7.85 (d, J=0.8 Hz, 1H), 8.03 (d, J=2.8 Hz, 2H), 8.41 (d, J=5.2 Hz, 1H), 8.78 (d, J=0.6 Hz, 1H).

### Example 429—Synthesis of (R)-1-(4-(1-Aminoethyl)Pyridin-2-yl)-N-(1-Methyl-1H-Indazol-7-yl)-1H-Pyrazole-4-Sulfonamide (I-500)

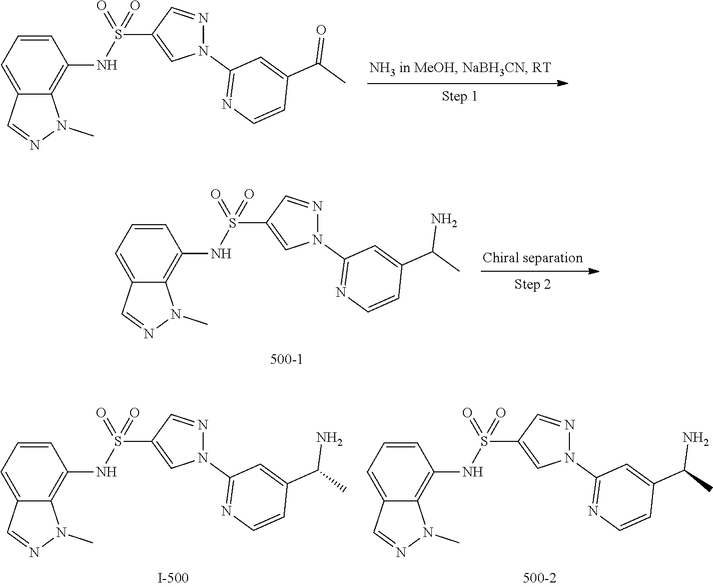

Step 1: Synthesis of 500-1: Into a 40-mL vial were added 1-(4-acetylpyridin-2-yl)-N-(1-methylindazol-7-yl)pyrazole-4-sulfonamide (300 mg, 0.757 mmol, 1 equiv) in NH₃(g) in MeOH (5 mL). The mixture was stirred at room temperature for 4 hours. Then, to the above mixture was added NaBH₃CN (95.11 mg, 1.514 mmol, 2 equiv) at room temperature. The resulting mixture was stirred overnight at room temperature. The resulting mixture was concentrated under reduced pressure. The resulting mixture was diluted with water (30 mL). The resulting mixture was extracted with EtOAc (3×30 mL). T he combined organic layers were concentrated under reduced pressure. The residue was purified by reversed-phase flash chromatography with the following conditions: column, C18 silica gel; mobile phase, MeCN in water (0.1% TFA), 20% to 30% gradient in 10 min; detector, UV 254 nm. This afforded 500-1 (150 mg, 44.88%) as a brown semi-solid.

Step 2: Synthesis of (R)-1-(4-(1-aminoethyl)pyridin-2-yl)-N-(1-methyl-1H-indazol-7-yl)-1H-pyrazole-4-sulfonamide (I-500): The 500-1 (150 mg) was purified by Prep-Chiral-HPLC with the following conditions (Column: CHIRALPAK IG, 2*25 cm, 5 μm; Mobile Phase A: MTBE(0.5% 2M NH₃-MeOH), Mobile Phase B: EtOH:DCM=1:1-HPLC; Flow rate: 20 mL/min; Gradient: 20% B to 20% B in 14 min; Wave Length: 220/254 nm; RT1(min): 6.75; RT2(min): 11.18; Sample Solvent: EtOH:DCM=1:1-HPLC; Injection Volume: 0.7 mL; Number of Runs: 4) to afford (R)-1-(4-(1-aminoethyl)pyridin-2-yl)-N-(1-methyl-1H-indazol-7-yl)-1H-pyrazole-4-sulfonamide (I-500, 36.1 mg, 23.03%) and 500-2 (24.1 mg, 15.44%) as an off-white solid. I-500: LC-MS: (ES, m/z): [M+H]⁺=398. ¹H-NMR: (400 MHz, Methanol-d4) δδ 1.57 (d, J=6.8 Hz, 3H), 4.37 (s, 3H), 4.42 (q, J=6.8 Hz, 1H), 6.74 (dd, J=7.3, 1.0 Hz, 1H), 6.92-6.98 (m, 2H), 7.43 (dd, J=5.2, 1.6 Hz, 1H), 7.71 (dd, J=8.2, 1.0 Hz, 1H), 7.87 (s, 1H), 8.03 (s, 1H), 8.12 (d, J=1.6 Hz, 1H), 8.47 (d, J=5.2 Hz, 1H), 8.80 (s, 1H). 500-2: LC-MS: (ES, m/z): [M+H]⁺=398. ¹H-NMR: (400 MHz, Methanol-d₄) δ 1.57 (d, J=6.8 Hz, 3H), 4.37 (s, 3H), 4.42 (q, J=6.8 Hz, 1H), 6.74 (dd, J=7.3, 1.0 Hz, 1H), 6.92-6.98 (m, 2H), 7.43 (dd, J=5.2, 1.6 Hz, 1H), 7.71 (dd, J=8.2, 1.0 Hz, 1H), 7.87 (s, 1H), 8.03 (s, 1H), 8.12 (d, J=1.6 Hz, 1H), 8.47 (d, J=5.2 Hz, 1H), 8.80 (s, 1H).

### Example 430—Synthesis of 1-(4-(1-Hydroxy-2-Methylpropan-2-yl)Pyridin-2-yl)-N-(1-Methyl-1H-Indazol-7-yl)-1H-Pyrazole-4-Sulfonamide (I-307)

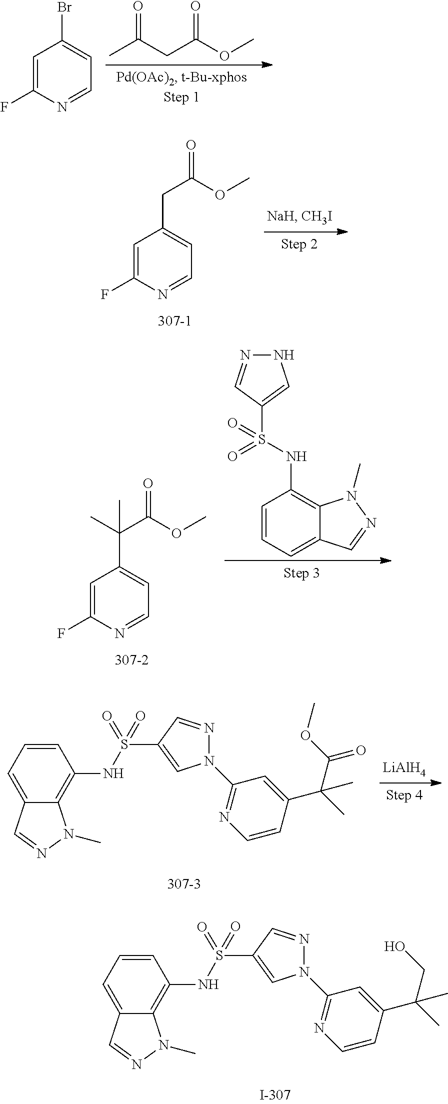

Step 1: Synthesis of 307-1: To a solution of 4-bromo-2-fluoropyridine (5.00 g, 28.4 mmol, 1.00 equiv) in toluene (200 ml) was added methyl acetoacetate (6.60 g, 56.8 mmol, 2.00 equiv), t-BuXPhos (1.21 g, 2.84 mmol, 0.10 equiv), Pd(OAc)₂ (0.32 g, 1.42 mmol, 0.05 equiv), and K₃PO₄ (24.1 g, 113 mmol, 4.00 equiv). The mixture was stirred overnight at 120° C. under a nitrogen atmosphere. The desired product could be detected by LCMS. The resulting mixture was concentrated under reduced pressure. The reaction was quenched with water at room temperature. The aqueous layer was extracted with CH₂Cl₂ (3×100 mL). The resulting mixture was filtered, and the filter cake was washed with DCM (3×200 mL). The filtrate was concentrated under reduced pressure. The residue was purified by silica gel column chromatography, eluting with PE/EA (10:1) to afford methyl 2-(2-fluoropyridin-4-yl)acetate (307-1, 1.2 g, 24%) as a brown-yellow oil. LC-MS: (ES, m/z): [M+H]=170.

Step 2: Synthesis of 307-2: A solution of methyl 2-(2-fluoropyridin-4-yl) acetate (1.20 g, 7.09 mmol, 1.00 equiv) in THE (12 ml) was treated with NaH (340 mg, 14.1 mmol, 2.00 equiv) for 30 minutes at 0° C. under a nitrogen atmosphere. Next, CH₃I (2.01 g, 14.1 mmol, 2.00 equiv) was added dropwise at 0° C. The resulting mixture was stirred for an additional 2 hours at room temperature. The desired product could be detected by LCMS. The reaction was quenched with saturated aqueous NH₄Cl at 0° C. The aqueous layer was extracted with CH₂Cl₂ (3×100 mL). The residue was purified by reversed-phase flash chromatography with the following conditions: column, C18 silica gel; mobile phase, MeCN in Water (10 mmol/L NH₄HCO₃), 50% to 70% gradient in 15 min; detector, UV 254 nm. This afforded methyl 2-(2-fluoropyridin-4-yl)-2-methylpropanoate (307-2, 1.00 g, 70%) as a white solid. LC-MS: (ES, m/z): [M+H]=198.

Step 3: Synthesis of 307-3: To a stirred mixture of methyl 2-(2-fluoropyridin-4-yl)-2-methylpropanoate (725 mg, 3.67 mmol, 1.20 equiv) and N-(1-methylindazol-7-yl)-1H-pyrazole-4-sulfonamide (850 mg, 3.06 mmol, 1.00 equiv) in DMF (20 mL) was added Cs₂CO₃ (2.99 g, 9.19 mmol, 3.00 equiv) at room temperature under a nitrogen atmosphere. The resulting mixture was stirred for 5 hours at 80° C. The desired product could be detected by LCMS. The reaction was quenched with the addition of water/ice (20 mL) at 0° C. The resulting mixture was extracted with CH₂Cl₂ (3×20 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 40% to 60% gradient in 10 min; detector, UV 254 nm. Methyl 2-methyl-2-(2-{4-[(1-methylindazol-7-yl)sulfamoyl]pyrazol-1-yl}pyridin-4-yl)propanoate (400 mg, 28%) was obtained as an off-white solid. LC-MS: (ES, m/z): [M+H]=455.

Step 4: Synthesis of 1-(4-(1-hydroxy-2-methylpropan-2-yl)pyridin-2-yl)-N-(1-methyl-1H-indazol-7-yl)-1H-pyrazole-4-sulfonamide (I-307). To a stirred mixture of methyl 2-methyl-2-(2-{4-[(1-methylindazol-7-yl) sulfamoyl] pyrazol-1-yl} pyridin-4-yl) propanoate (400 mg, 0.88 mmol, 1.00 equiv) in THE (5 mL) was added LiAlH₄ (100 mg, 2.640 mmol, 3.00 equiv) dropwise at 0° C. under a nitrogen atmosphere. The resulting mixture was stirred overnight at room temperature under a nitrogen atmosphere. The reaction was quenched with saturated aqueous NH₄Cl at room temperature. The resulting mixture was extracted with EtOAc (3×5 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 20% to 40% gradient in 10 min; detector, UV 254 nm. This product, 1-[4-(1-hydroxy-2-methylpropan-2-yl)pyridin-2-yl]-N-(1-methyl-1H-indazol-7-yl)-1H-pyrazole-4-sulfonamide (I-307, 23.0 mg, 6%) was obtained as an off-white solid. LC-MS: (ES, m/z): [M+H]=427. ¹H-NMR: (400 MHz, DMSO-d₆) δ 1.28 (s, 6H), 3.51 (d, J=4.6 Hz, 2H), 4.29 (s, 3H), 4.89 (t, J=5.4 Hz, 1H), 6.70 (d, J=7.4 Hz, 1H), 6.98 (t, J=7.8 Hz, 1H), 7.49 (dd, J=5.4, 1.8 Hz, 1H), 7.70 (d, J=8.0 Hz, 1H), 7.96 (d, J=1.6 Hz, 1H), 8.01 (s, 1H), 8.09 (s, 1H), 8.42 (d, J=5.4 Hz, 1H), 8.79 (s, 1H), 10.02 (s, 1H).

### Example 431—Synthesis of (R)-1-(4-(3-Hydroxytetrahydrofuran-3-yl)Pyridin-2-yl)-N-(1-Methyl-1H-Indazol-7-yl)-1H-Pyrazole-4-Sulfonamide (I-312) AND (S)-1-(4-(3-Hydroxytetrahydrofuran-3-yl)Pyridin-2-yl)-N-(1-Methyl-1H-Indazol-7-yl)-1H-Pyrazole-4-Sulfonamide (I-310)

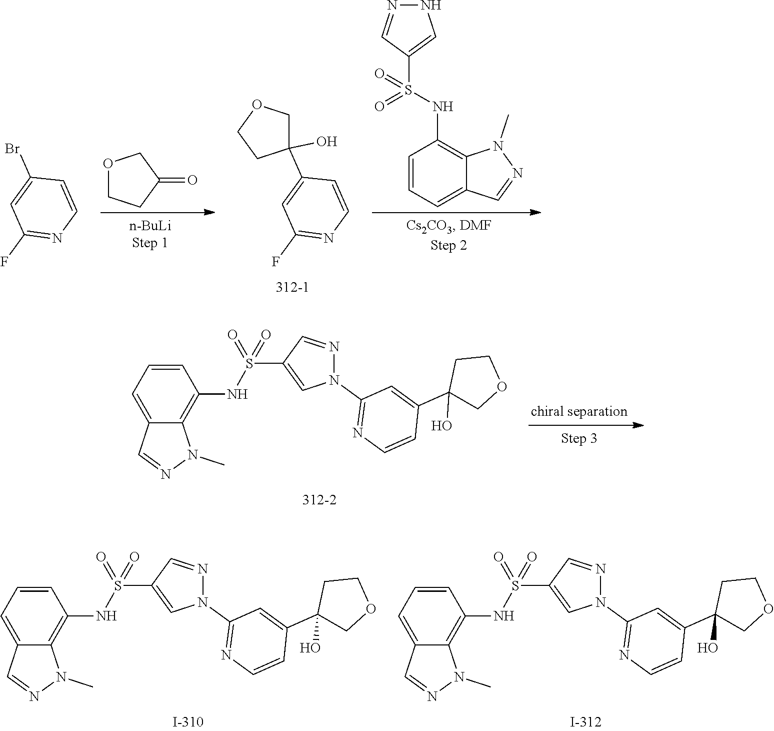

Step 1: Synthesis of 312-1: A solution of 4-bromo-2-fluoropyridine (3 g, 17.047 mmol, 1 equiv) in THE (20 mL) was treated with n-BuLi (3.28 g, 51.141 mmol, 3 equiv) for 1 hour at −78° C. under a nitrogen atmosphere after which dihydrofuran-3-one (4.40 g, 51.141 mmol, 3 equiv) was added in portions at −78° C. The resulting mixture was stirred for an additional 2 hours at room temperature. The residue was acidified to pH 5 with concentrated HCl. The resulting mixture was extracted with EtOAc (5×20 mL). The combined organic layers were washed with water (1×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 20% to 40% gradient in 15 min; detector, UV 254 nm. The crude product (3 g) was purified by Prep-HPLC with the following conditions (Column: DAICEL DCpak P4VP 3*25 cm, 5 μm; Mobile Phase A: CO₂, Mobile Phase B: IPA(0.1% 2M NH₃-MeOH); Flow rate: 60 mL/min; Gradient: isocratic 14% B; Column Temperature(° C.): 35; Back Pressure(bar): 100; Wave Length: 254 nm; RT1(min): 9.10; RT2(min): 11.22; Sample Solvent: MeOH-HPLC; Injection Volume: 2 mL; Number Of Runs: 18.0) to afford 312-1 (1.5 g, 48.04%) as a yellow oil.

Step 2: Synthesis of 312-2: A mixture of 312-1 (200 mg, 1.092 mmol, 1 equiv), N-(1-methylindazol-7-yl)-1H-pyrazole-4-sulfonamide (363.31 mg, 1.310 mmol, 1.2 equiv), and Cs₂CO₃ (1.78 g, 5.460 mmol, 5 equiv) in DMSO (5 mL) was stirred overnight at 80° C. under a nitrogen atmosphere. The resulting mixture was diluted with water (20 mL). The resulting mixture was extracted with EtOAc (4×20 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 30% gradient in 10 min; detector, UV 254 nm. Product 312-2 (150 mg, 31.19%) was obtained as a yellow solid.

Step 3: Synthesis of (R)-1-(4-(3-hydroxytetrahydrofuran-3-yl)pyridin-2-yl)-N-(1-methyl-1H-indazol-7-yl)-1H-pyrazole-4-sulfonamide (I-312) and (S)-1-(4-(3-hydroxytetrahydrofuran-3-yl)pyridin-2-yl)-N-(1-methyl-1H-indazol-7-yl)-1H-pyrazole-4-sulfonamide (I-310). Compound 312-2 (150 mg, 0.341 mmol, 1 equiv) was purified by Prep-HPLC with the following conditions (Column: CHIRALPAKIG3; Mobile Phase A: Hex (0.2% TFA): (MeOH:DCM=1:1)=75:25; Flow rate: 1 mL/min; Gradient: isocratic; Injection Volume: 0.2 mL) to afford (S)-1-(4-(3-hydroxytetrahydrofuran-3-yl)pyridin-2-yl)-N-(1-methyl-1H-indazol-7-yl)-1H-pyrazole-4-sulfonamide (I-310) (31.2 mg) as a white solid and (R)-1-(4-(3-hydroxytetrahydrofuran-3-yl)pyridin-2-yl)-N-(1-methyl-1H-indazol-7-yl)-1H-pyrazole-4-sulfonamide (I-312) (30.5 mg) as a white solid. I-310: LC-MS: (ES, m/z): [M+H]⁺=441. ¹H-NMR: ¹H NMR (400 MHz, Methanol-d₄) δ 2.28 (dt, J=13.2, 5.0 Hz, 1H), 2.43 (dt, J=13.0, 9.0 Hz, 1H), 3.94 (s, 2H), 4.13-4.24 (m, 2H), 4.38 (s, 3H), 4.59 (s, 1H), 6.76 (dd, J=6.8, 1.8 Hz, 1H), 6.96 (t, J=7.8 Hz, 1H), 7.52 (dd, J=5.2, 1.8 Hz, 1H), 7.67 (d, J=8.2 Hz, 1H), 7.86 (s, 1H), 8.01 (s, 1H), 8.21 (d, J=1.8 Hz, 1H), 8.41 (d, J=5.2 Hz, 1H), 8.78 (s, 1H). I-312: LC-MS: (ES, m/z): [M+H]⁺=441. ¹H-NMR: (400 MHz, Methanol-d₄) δ 2.28 (dt, J=13.2, 5.0 Hz, 1H), 2.43 (dt, J=13.0, 9.0 Hz, 1H), 3.94 (s, 2H), 4.14-4.24 (m, 2H), 4.37 (s, 3H), 4.59 (s, 1H), 6.75 (dd, J=6.8, 1.8 Hz, 1H), 6.96 (t, J=7.8 Hz, 1H), 7.52 (dd, J=5.2, 1.8 Hz, 1H), 7.69 (d, J=8.2 Hz, 1H), 7.86 (s, 1H), 8.02 (s, 1H), 8.21 (d, J=1.8 Hz, 1H), 8.41 (d, J=5.2 Hz, 1H), 8.78 (s, 1H).

### Example 432—Synthesis of (R)-1-(4-(1-Hydroxypropyl)Pyridin-2-yl)-N-(1-Methyl-1H-Indazol-7-yl)-1H-Pyrazole-4-Sulfonamide (I-278)

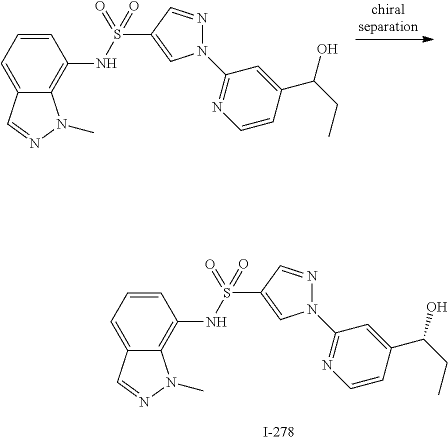 [SMILES: CCC(O)c1ccnc(-n2cc(S(=O)(=O)Nc3cccc4cnn(C)c34)cn2)c1 ; CC[C@@H](O)c1ccnc(-n2cc(S(=O)(=O)Nc3cccc4cnn(C)c34)cn2)c1]

Crude 1-(4-(1-hydroxypropyl)pyridin-2-yl)-N-(1-methyl-1H-indazol-7-yl)-1H-pyrazole-4-sulfonamide (120 mg crude) was purified by Chiral-HPLC with the following conditions (Column: CHIRAKPAKAD3; Mobile Phase A: Hex(0.2% TFA): EtOH=60:40; Flow rate: 1 mL/min; Gradient: isocratic; Injection Volume: 3 μL) to afford (R)-1-(4-(1-hydroxypropyl) pyridin-2-yl)-N-(1-methyl-1H-indazol-7-yl)-1H-pyrazole-4-sulfonamide (I-278, 33.9 mg, 13.77%) as a light-yellow solid. LC-MS: (ES, m/z): [M+H]⁺=412. ¹H-NMR: (400 MHz, DMSO-d6) δ 0.87 (t, J=7.2 Hz, 3H), 1.71-1.99 (m, 2H), 4.28 (s, 3H), 4.61-4.68 (t, J=5.2 Hz, 1H), 5.56 (s, 1H), 6.71 (d, J=7.2 Hz, 1H), 6.99 (t, J=8.0 Hz, 1H), 7.42 (d, J=5.2 Hz, 1H), 7.71 (d, J=8.0 Hz, 1H), 8.00 (d, J=8.8 Hz, 1H), 8.05 (s, 1H), 8.09 (s, 1H), 8.43 (d, J=5.2 Hz, 1H), 8.79 (s, 1H), 10.03 (s, 1H).

### Example 433—Synthesis of 1-[4-(1-Hydroxycyclopentyl)Pyridin-2-yl]-N-(3-Methyl-1H-Indazol-4-yl)Pyrazole-4-Sulfonamide (I-246)

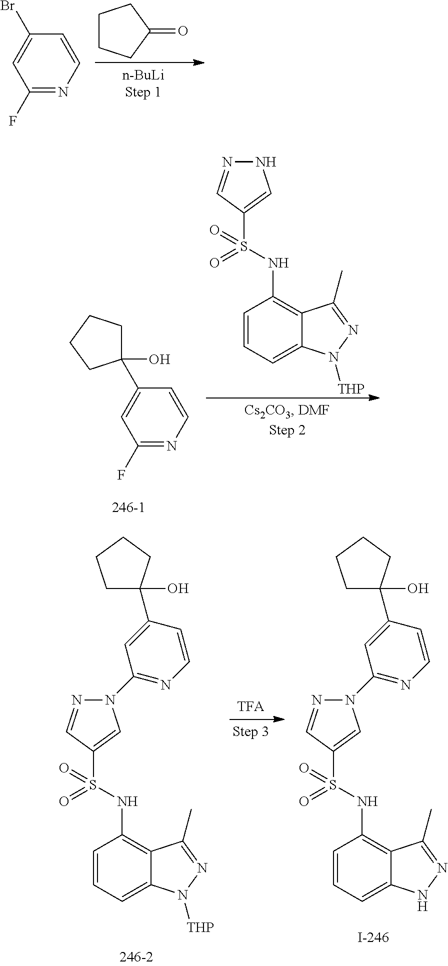

Step 1: Synthesis of 246-1: A solution of 4-bromo-2-fluoropyridine (1 g, 5.682 mmol, 1 equiv) in THE (10 mL) was treated with n-butyllithium (3.41 mL, 8.523 mmol, 1.5 equiv) for 30 min at −70° C. under nitrogen atmosphere followed by the addition of cyclopentanone (0.72 g, 8.523 mmol, 1.5 equiv) dropwise at −70° C. The resulting mixture was stirred for additional 2 h at room temperature. The reaction was quenched by the addition of water (20 mL) at room temperature. The resulting mixture was extracted with CH₂Cl₂ (5×10 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 80% gradient in 25 min; detector, UV 254 nm. This resulted in 1-(2-fluoropyridin-4-yl)cyclopentan-1-ol (246-1, 490 mg, 40.45%) as a yellow oil.

Step 2: Synthesis of 246-2: A solution of N-[3-methyl-1-(oxan-2-yl)indazol-4-yl]-1H-pyrazole-4-sulfonamide (100 mg, 0.277 mmol, 1 equiv), Cs₂CO₃ (180.30 mg, 0.554 mmol, 2 equiv) and 1-(2-fluoropyridin-4-yl)cyclopentan-1-ol (60.17 mg, 0.332 mmol, 1.2 equiv) in DMSO (5 mL) was stirred for overnight at 140° C. under nitrogen atmosphere. The resulting mixture was diluted with water (10 mL) and extracted with CH₂Cl₂ (3×10 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 20% to 80% gradient in 25 min; detector, UV 254 nm. This resulted in 1-[4-(1-hydroxycyclopentyl)pyridin-2-yl]-N-[3-methyl-1-(oxan-2-yl) indazol-4-yl]pyrazole-4-sulfonamide (246-2, 150 mg, 88.17%) as a yellow oil.

Step 3: Synthesis of I-246: A solution of 1-[4-(1-hydroxycyclopentyl)pyridin-2-yl]-N-[3-methyl-1-(oxan-2-yl)indazol-4-yl] pyrazole-4-sulfonamide (120 mg, 0.230 mmol, 1 equiv) and TFA (4 mL) in DCM (2 mL) was stirred for 3 h at room temperature under nitrogen atmosphere. The resulting mixture was diluted with water (10 mL) and extracted with CH₂Cl₂ (3×10 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 0% to 100% gradient in 25 min; detector, UV 254 nm. This resulted in 1-[4-(1-hydroxycyclopentyl)pyridin-2-yl]-N-(3-methyl-1H-indazol-4-yl)pyrazole-4-sulfonamide (I-246, 44.3 mg, 41.80%) as a white solid. LC-MS: (ES, m/z): [M+1]=439. ¹H NMR (400 MHz, DMSO-d₆) δ 1.78-1.80 (m, 2H), 1.81-1.83 (m, 6H), 2.59 (s, 3H), 5.25 (s, 1H), 6.56 (d, J=7.2 Hz, 1H), 7.13 (t, J=7.6 Hz, 1H), 7.22 (d, J=8.4 Hz, 1H), 7.48 (dd, J=1.6, 5.2 Hz, 1H), 7.97 (s, 1H), 8.08 (d, J=1.6 Hz, 1H), 8.40 (d, J=5.2 Hz, 1H), 8.74 (s, 1H), 12.61 (s, 1H).

### Example 434—Synthesis of N-(1-Methyl-1H-Indazol-7-yl)-1-(4-(Piperidin-1-yl)Pyridin-2-yl)-1H-Pyrazole-4-Sulfonamide (I-250)

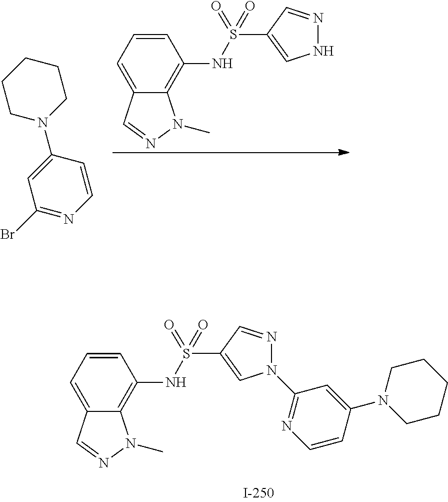 [SMILES: Brc1cc(N2CCCCC2)ccn1 ; Cn1ncc2cccc(NS(=O)(=O)c3cn[nH]c3)c21 ; Cn1ncc2cccc(NS(=O)(=O)c3cnn(-c4cc(N5CCCCC5)ccn4)c3)c21]

Synthesis of N-(1-methyl-1H-indazol-7-yl)-1-(4-(piperidin-1-yl)pyridin-2-yl)-1H-pyrazole-4-sulfonamide (I-250): A solution of 2-bromo-4-(piperidin-1-yl)pyridine (100 mg, 0.415 mmol, 1 equiv) in dioxane (2 mL) was treated with N-(1-methylindazol-7-yl)-1H-pyrazole-4-sulfonamide (138.00 mg, 0.498 mmol, 1.2 equiv) for 2 min at room temperature under nitrogen atmosphere followed by the addition of EPhos (44.36 mg, 0.083 mmol, 0.2 equiv), EPhos Pd G4 (76.19 mg, 0.083 mmol, 0.2 equiv), and Cs₂CO₃ (405.36 mg, 1.245 mmol, 3 equiv) in portions at 90° C. The resulting mixture was stirred for additional 2 h at 90° C. Desired product could be detected by LCMS. The resulting mixture was concentrated under reduced pressure. The residue was purified by reversed-phase flash chromatography with the following conditions: column, C18 silica gel; mobile phase, MeCN in Water (0.1% FA), 40% to 70% gradient in 150 min; detector, UV 254 nm. This resulted in N-(1-methylindazol-7-yl)-1-[4-(piperidin-1-yl)pyridin-2-yl]pyrazole-4-sulfonamide (9.5 mg, 5.20%) as a white solid. LC-MS: (ES, m/z): [M+1]=438. ¹H-NMR: (400 MHz, DMSO-d6) δ 1.56-1.64 (m, 6H), 3.46 (t, J=5.2 Hz, 4H), 4.29 (s, 3H), 6.70 (d, J=7.2 Hz, 1H), 6.87 (dd, J=6.4, 2.4 Hz, 1H), 6.96 (t, J=7.6 Hz, 1H), 7.28 (d, J=2.4 Hz, 1H), 7.64 (d, J=8.0 Hz, 1H), 7.92 (s, 1H), 8.03 (d, J=6.0 Hz, 1H), 8.06 (s, 1H), 8.70 (s, 1H), 9.96 (s, 1H).

### Example 435—Synthesis of 1-[4-(1-Hydroxycyclopentyl) Pyridin-2-yl]-N-(1-Methylindazol-7-yl) Pyrazole-4-Sulfonamide (I-247)

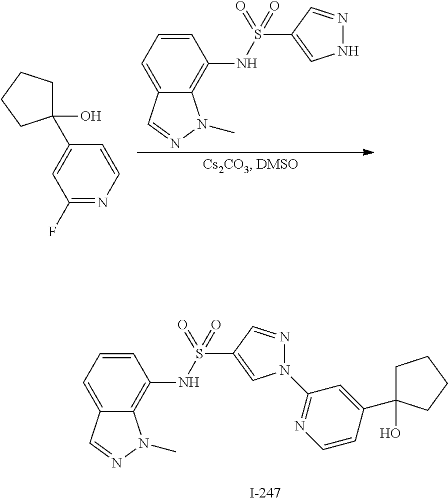 [SMILES: OC1(c2ccnc(F)c2)CCCC1 ; Cn1ncc2cccc(NS(=O)(=O)c3cn[nH]c3)c21 ; Cn1ncc2cccc(NS(=O)(=O)c3cnn(-c4cc(C5(O)CCCC5)ccn4)c3)c21]

Step 1: Synthesis of -[4-(1-hydroxycyclopentyl) pyridin-2-yl]-N-(1-methylindazol-7-yl) pyrazole-4-sulfonamide (I-247): To a stirred solution of 1-(2-fluoropyridin-4-yl) cyclopentan-1-ol (71.88 mg, 0.397 mmol, 1.1 equiv) and N-(1-methylindazol-7-yl)-1H-pyrazole-4-sulfonamide (100 mg, 0.361 mmol, 1.00 equiv) in DMSO (1 mL) was added Cs₂CO₃ (352.49 mg, 1.083 mmol, 3 equiv) at room temperature under nitrogen atmosphere. The resulting mixture was stirred for overnight at 140° C. under nitrogen atmosphere. The resulting mixture was diluted with water (10 mL). The resulting mixture was extracted with EtOAc (3×5 mL). The combined organic layers were washed with brine (1×10 mL), dried over anhydrous Na₂SO₄. After filtration, the filtrate was concentrated under reduced pressure. The crude product (70 mg) was purified by prep-HPLC with the following conditions (column: XBridge Shield RP18 OBD column, 30*150 mm, 5 μm; mobile phase A: water (10 mmol/L NH₄HCO₃+0.05% NH₃H₂O), mobile phase B: ACN; flow rate: 60 mL/min; gradient: 13% B to 40% B in 7 min; wavelength: 254 nm/220 nm; RT1(min): 7; number of runs: 3) to afford 1-[4-(1-hydroxycyclopentyl) pyridin-2-yl]-N-(1-methylindazol-7-yl) pyrazole-4-sulfonamide (I-247, 9.3 mg, 5.85%) as a white solid. LC-MS: (ES, m/z): [M+1]=439. ¹H NMR (400 MHz, DMSO-d₆) δ 1.70-1.90 (m, 8H), 4.29 (s, 3H), 5.27 (s, 1H), 6.71 (d, J=7.2 Hz, 1H), 6.95 (t, J=7.6 Hz, 1H), 7.50 (d, J=5.6 Hz, 1H), 7.63 (d, J=8.4 Hz, 1H), 7.99 (s, 1H), 8.06 (s, 1H), 8.13 (d, J=14.0 Hz, 1H), 8.41 (d, J=5.2 Hz, 1H), 8.77 (s, 1H), 10.05 (s, 1H).

### Example 436—Synthesis of 1-(4-(3-Fluoro-1-Methylazetidin-3-yl)Pyridin-2-yl)-N-(1-Methyl-1H-Indazol-7-yl)-1H-Pyrazole-4-Sulfonamide (I-251)

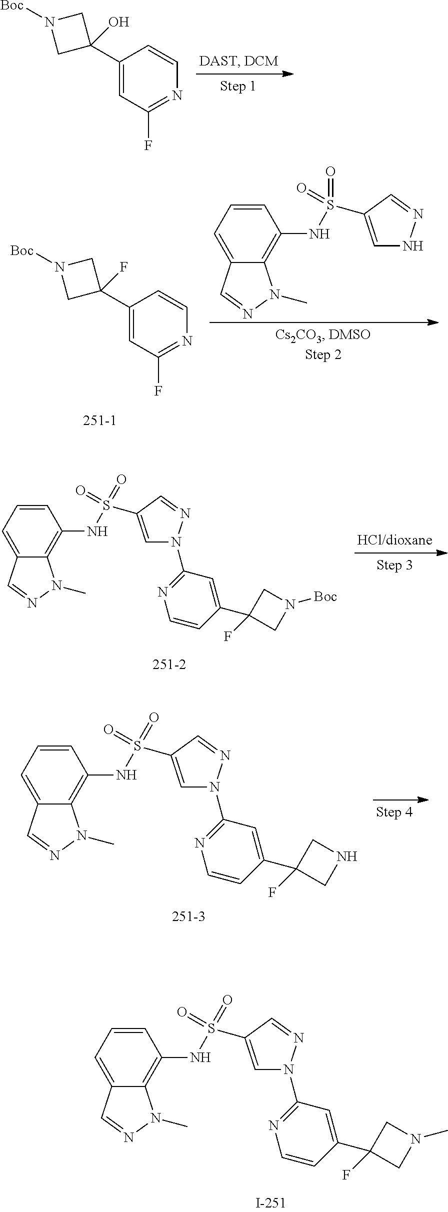

Step 1: Synthesis of 251-1: A solution of tert-butyl 3-(2-fluoropyridin-4-yl)-3-hydroxyazetidine-1-carboxylate (472 mg, 1.759 mmol, 1 equiv) and DAST (709 mg, 4.399 mmol, 2.50 equiv) in DCM (15 mL) was stirred for 3 h at 0° C. under nitrogen atmosphere. The reaction was quenched by the addition of sat. NaHCO₃ (aq.) (30 mL) at 0° C. The resulting mixture was extracted with EtOAc (3×20 mL). The combined organic layers were washed with brine (40 mL), dried over anhydrous Na₂SO₄. After filtration, the filtrate was concentrated under reduced pressure to afford 251-1(450 mg, 70.98%) as a light yellow oil.

Step 2: Synthesis of 251-2: To a stirred mixture of 251-1 (425 mg, 1.572 mmol, 1 equiv) and Cs₂CO₃ (1560 mg, 4.788 mmol, 3.04 equiv) in DMSO (15 mL) was added N-(1-methylindazol-7-yl)-1H-pyrazole-4-sulfonamide (500 mg, 1.803 mmol, 1.15 equiv) in portions at 80° C. under nitrogen atmosphere. The resulting mixture was stirred overnight at 80° C. under nitrogen atmosphere. After the reaction was completed, the resulting mixture was diluted with water (20 mL). The resulting mixture was extracted with EtOAc (3×20 mL). The combined organic layers were washed with brine (40 mL), dried over anhydrous Na₂SO₄. After filtration, the filtrate was concentrated under reduced pressure. The residue was purified by reversed-phase flash chromatography with the following conditions: column, C₁₈ silica gel; mobile phase, ACN in water (10 mmol/L NH₄HCO₃), 20% to 60% gradient in 30 min; detector, UV 254 nm to afford 251-2 (420 mg, 50.63%) as an off-white solid.

Step 3: Synthesis of 251-3: A solution of 251-2 (410 mg, 0.777 mmol, 1 equiv) and HCl of 1,4-dioxane solution (10 mL) was stirred for 1.5 h at room temperature under nitrogen atmosphere. The resulting mixture was concentrated under reduced pressure to afford 251-3 (456 mg, 96.09%) as a light yellow solid.

Step 4: Synthesis of I-251: A solution of 251-3 (127 mg, 0.099 mmol, 1 equiv) in DCM (7.5 mL) and MeOH (7.5 mL) was treated with HCHO (14.87 mg, 0.495 mmol, 5 equiv) and AcOH (29.74 mg, 0.495 mmol, 5 equiv) for 15 min at room temperature under nitrogen atmosphere followed by the addition of NaBH₃CN (18.67 mg, 0.297 mmol, 3 equiv) in portions at 0° C. The mixture was stirred for 1 h at room temperature under nitrogen atmosphere. The resulting mixture was diluted with water (15 mL). The resulting mixture was extracted with EtOAc (4×25 mL). The combined organic layers were washed with brine (40 mL), dried over anhydrous Na₂SO₄. After filtration, the filtrate was concentrated under reduced pressure. The crude product (200 mg) was purified by prep-HPLC with the following conditions (column: XBridge Prep OBD C₁₈ column, 30*150 mm, 5 μm; mobile phase A: water(10 mmol/L NH₄HCO₃+0.05% NH₃H₂O), mobile phase B: ACN; flow rate: 60 mL/min; gradient: 15% B to 35% B in 7 min; wavelength: 220 nm; RT1(min): 8.87) to afford 1-(4-(3-fluoro-1-methylazetidin-3-yl)pyridin-2-yl)-N-(1-methyl-1H-indazol-7-yl)-1H-pyrazole-4-sulfonamide (I-251, 40.3 mg, 30.11%) as an off-white solid. LC-MS: (ES, m/z): [M+H]⁺=442. ¹H-NMR: (400 MHz, DMSO, ppm): 2.44 (s, 3H), 3.54 (d, J=9.2 Hz, 1H), 3.58 (d, J=9.6 Hz, 1H), 3.72 (d, J=13.6, 2H), 4.29 (s, 2H), 6.72 (d, J=7.6 Hz, 1H), 6.96 (s, 1H), 7.62 (t, J=4.0 Hz, 1H), 7.63 (s, 1H), 8.06 (d, J=12.4 Hz, 2H), 8.18 (s, 1H), 8.58 (d, J=5.2 Hz, 1H), 8.81 (s, 1H) δ 10.06 (s, 1H).

### Example 437—Synthesis of 1-[4-(1-Hydroxycyclobutyl)Pyridin-2-yl]-N-(3-Methyl-1H-Indazol-4-yl)Pyrazole-4-Sulfonamide (I-255)

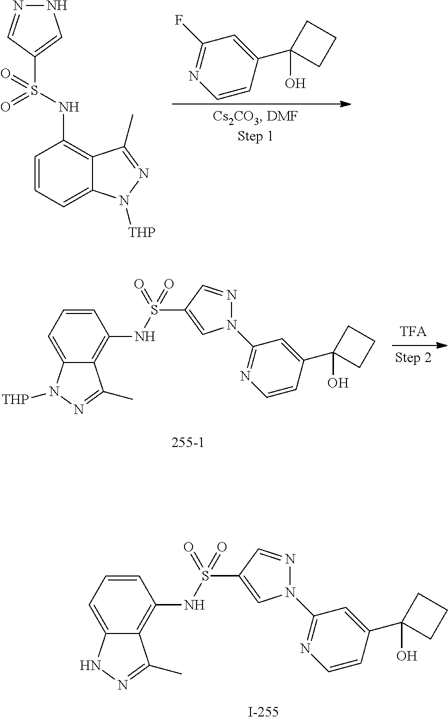

Step 1: Synthesis of 255-1: A solution of N-[3-methyl-1-(oxan-2-yl)indazol-4-yl]-1H-pyrazole-4-sulfonamide (100 mg, 0.277 mmol, 1 equiv), 1-(2-fluoropyridin-4-yl)cyclobutan-1-ol (69.39 mg, 0.416 mmol, 1.5 equiv) and Cs₂CO₃ (270.45 mg, 0.831 mmol, 3 equiv) in DMF (0.5 mL) was stirred for 24 h at 80° C. under a nitrogen atmosphere. The resulting mixture was diluted with water (10 mL). The resulting mixture was extracted with DCM (3×20 mL). The combined organic layers were washed with brine (1×50 mL), dried over anhydrous Na₂SO₄. The resulting mixture was used in the next step directly without further purification.

Step 2: Synthesis of 1-[4-(1-hydroxycyclobutyl)pyridin-2-yl]-N-(3-methyl-1H-indazol-4-yl)pyrazole-4-sulfonamide (I-255): A mixture of 1-[4-(1-hydroxycyclobutyl)pyridin-2-yl]-N-[3-methyl-1-(oxan-2-yl)indazol-4-yl] pyrazole-4-sulfonamide (100 mg, 0.197 mmol, 1 equiv) and trifluoroacetaldehyde (1.5 mL) in DCM (1.5 mL) was stirred for 2 h at room temperature under air atmosphere. The resulting mixture was diluted with water (10 mL). The resulting mixture was extracted with DCM (3×20 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. This resulted in 1-[4-(1-hydroxycyclobutyl)pyridin-2-yl]-N-(3-methyl-1H-indazol-4-yl)pyrazole-4-sulfonamide (I-255, 14.3 mg, 16.96%) as a white solid. LC-MS: (ES, m/z): [M+1]=425. ¹H-NMR: (400 MHz, DMSO-d6) δ 1.78-1.81 (m, 1H), 1.99-2.02 (m, 1H), 2.33-2.44 (m, 4H), 2.59 (s, 3H), 6.02 (s, 1H), 6.55 (d, J=7.2 Hz, 1H), 7.15 (t, J=7.6 Hz, 1H), 7.28 (s, 1H), 7.57 (dd, J=1.6, 5.2 Hz, 1H), 8.00 (s, 1H), 8.05 (d, J=1.6 Hz, 1H), 8.46 (d, J=5.2 Hz, 1H), 8.77 (s, 1H), 9.89 (s, 1H), 12.69 (s, 1H).

### Example 438—Synthesis of 1-[4-(1-Hydroxycyclohexyl) Pyridin-2-yl]-N-(1-Methylindazol-7-yl)Pyrazole-4-Sulfonamide (I-256)

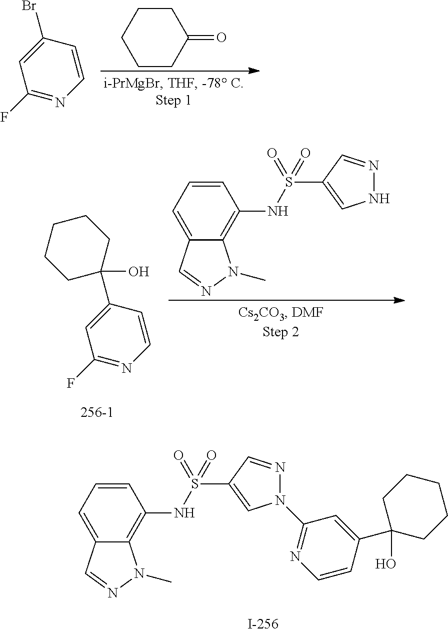

Step 1: Synthesis of 256-1: To a stirred solution of 4-bromo-2-fluoropyridine (1 g, 5.682 mmol, 1 equiv) in THE (10 mL) was added i-PrMgBr (6.56 mL, 8.523 mmol, 1.5 equiv) dropwise at −78° C. under nitrogen atmosphere. The resulting mixture was stirred for 40 min at −78° C. under nitrogen atmosphere. To the above mixture was added cyclohexanone (0.84 g, 8.523 mmol, 1.5 equiv). The resulting mixture was stirred for additional 2 h at room temperature. The reaction was quenched with water (10 mL) at room temperature. The aqueous layer was extracted with CH₂Cl₂ (3×10 mL). The combined organic layers were washed with brine (20 mL), dried over anhydrous Na2SO4. After filtration, the filtrate was concentrated under reduced pressure. The residue was purified by Prep-TLC (PE/EA=3:1) to afford 1-(2-fluoropyridin-4-yl) cyclohexan-1-ol (256-1, 220 mg, 15.86%) as an off-white solid.

Step 2: Synthesis of 1-[4-(1-hydroxycyclohexyl) pyridin-2-yl]-N-(1-methylindazol-7-yl)pyrazole-4-sulfonamide (I-256): To a stirred solution of 1-(2-fluoropyridin-4-yl) cyclohexan-1-ol (77.45 mg, 0.397 mmol, 1.1 equiv) and N-(1-methylindazol-7-yl)-1H-pyrazole-4-sulfonamide (100 mg, 0.361 mmol, 1.00 equiv) in DMF (2 mL) was added Cs₂CO₃ (352.49 mg, 1.083 mmol, 3 equiv) at room temperature under nitrogen atmosphere. The resulting mixture was stirred for overnight at 100° C. under nitrogen atmosphere. The resulting mixture was diluted with water (10 mL). The resulting mixture was extracted with EtOAc (3×10 mL). The combined organic layers were washed with brine (1×20 mL), dried over anhydrous Na₂SO₄. After filtration, the filtrate was concentrated under reduced pressure. The resulting mixture was concentrated under vacuum. The crude product (80 mg) was purified by prep-HPLC with the following conditions (column: XBridge Shield RP18 OBD column, 30*150 mm, 5 μm; mobile phase A: water (10 mmol/L NH₄HCO₃+0.05% NH₃H₂O), mobile phase B: ACN; flow rate: 60 mL/min; gradient: 13% B to 40% B in 7 min; wavelength: 254 nm/220 nm; RT1(min): 7; number of runs: 3) to afford 1-[4-(1-hydroxycyclohexyl) pyridin-2-yl]-N-(1-methylindazol-7-yl)pyrazole-4-sulfonamide (I-256, 14.5 mg, 8.87%) as a white solid. LC-MS: (ES, m/z): [M+1]=453. ¹H-NMR: ¹H NMR (400 MHz, DMSO-d₆) δ 1.33-1.75 (m, 10H), 4.29 (s, 3H), 5.20 (s, 1H), 6.71 (dd, J=1.2, 7.2 Hz, 1H), 6.98 (t, J=7.6 Hz, 1H), 7.54 (dd, J=1.6, 5.2 Hz, 1H), 7.69 (dd, J=1.2, 8.0 Hz, 1H), 8.01 (d, J=0.8 Hz, 1H), 8.09 (s, 1H), 8.12 (d, J=1.6 Hz, 1H), 8.43 (d, J=5.2 Hz, 1H), 8.79 (d, J=0.8 Hz, 1H), 10.03 (s, 1H).

### Example 439—Sythesis of N-(1-Methylindazol-7-yl)-1-[4-(Oxetan-3-yl) Pyridin-2-yl]Pyrazole-4-Sulfonamide (I-264)

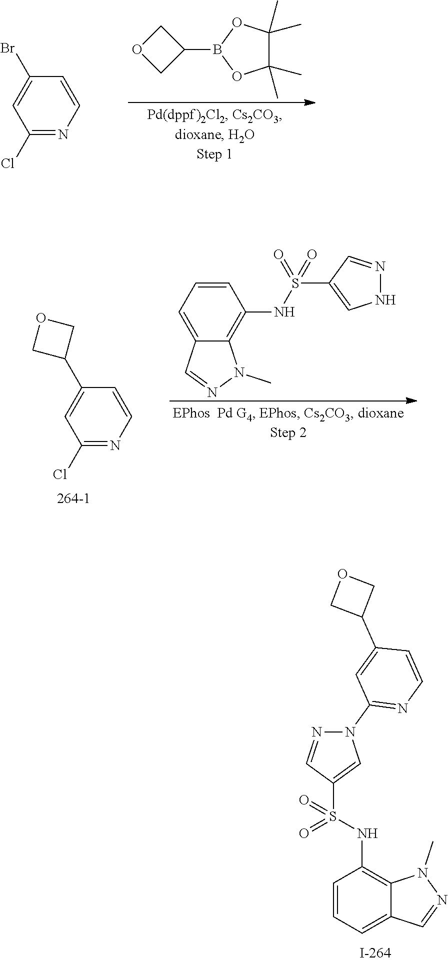 [SMILES: Clc1cc(Br)ccn1 ; CC1(C)OB(C2COC2)OC1(C)C ; Clc1cc(C2COC2)ccn1 ; Cn1ncc2cccc(NS(=O)(=O)c3cn[nH]c3)c21 ; Cn1ncc2cccc(NS(=O)(=O)c3cnn(-c4cc(C5COC5)ccn4)c3)c21]

Step 1: Synthesis of 264-1: To a stirred solution of 4-bromo-2-chloropyridine (1254.77 mg, 6.521 mmol, 1.2 equiv) and 4,4,5,5-tetramethyl-2-(oxetan-3-yl)-1,3,2-dioxaborolane (1000 mg, 5.434 mmol, 1.00 equiv) in H₂O (6 mL) and dioxane (24 mL) were added Pd(dppf)Cl₂ (397.58 mg, 0.543 mmol, 0.1 equiv) and Cs₂CO₃ (3540.74 mg, 10.868 mmol, 2 equiv) in portions at room temperature under nitrogen atmosphere. The resulting mixture was stirred for 2 h at 90° C. under nitrogen atmosphere. The resulting mixture was diluted with water (80 mL). The resulting mixture was extracted with EtOAc (3×150 mL). The combined organic layers were washed with brine (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% TFA), 35% to 50% gradient in 12 min; detector, UV 254 nm. The resulting mixture was concentrated under reduced pressure. This resulted in 2-chloro-4-(oxetan-3-yl) pyridine (264-1, 200 mg, 20.40%) as a brown solid.

Step 2: Synthesis of N-(1-methylindazol-7-yl)-1-[4-(oxetan-3-yl) pyridin-2-yl]pyrazole-4-sulfonamide (I-264): To a stirred mixture of 2-chloro-4-(oxetan-3-yl) pyridine (73.40 mg, 0.433 mmol, 1.2 equiv) and N-(1-methylindazol-7-yl)-1H-pyrazole-4-sulfonamide (100 mg, 0.361 mmol, 1.00 equiv) in dioxane (5 mL) were added EPhos (19.29 mg, 0.036 mmol, 0.1 equiv), EPhos Pd G4 (33.13 mg, 0.036 mmol, 0.1 equiv) and Cs₂CO₃ (352.49 mg, 1.083 mmol, 3 equiv) in portions at room temperature under nitrogen atmosphere. The resulting mixture was stirred for 2 h at 90° C. under nitrogen atmosphere. The resulting mixture was diluted with water (20 mL). The resulting mixture was extracted with EtOAc (3×50 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% TFA), 30% to 40% gradient in 8 min; detector, UV 254 nm. This resulted in N-(1-methylindazol-7-yl)-1-[4-(oxetan-3-yl) pyridin-2-yl] pyrazole-4-sulfonamide (I-264, 4.8 mg, 3.09%) as an off-white solid. LC-MS: (ES, m/z): [M+H]⁺=411. ¹H-NMR: (400 MHz, DMSO, ppm) δ 4.29 (s, 3H), 4.33 (m, 1H), 4.66 (t, J=8 Hz, 2H), 5.01 (t, J=8 Hz, 2H), 6.71 (d, J=7.4 Hz, 1H), 6.98 (t, J=8 Hz, 1H), 7.47 (d, J=4 Hz, 1H), 7.70 (d, J=8.0 Hz, 1H), 8.02 (d, J=4 Hz, 2H), 8.09 (s, 1H), 8.52 (d, J=4 Hz, 1H), 8.81 (s, 1H), 10.04 (s, 1H).

### Example 440—Synthesis of N-(1-Methylindazol-7-yl)-1-[4-(Oxetan-2-yl) Pyridin-2-yl] Pyrazole-4-Sulfonamide (I-265)

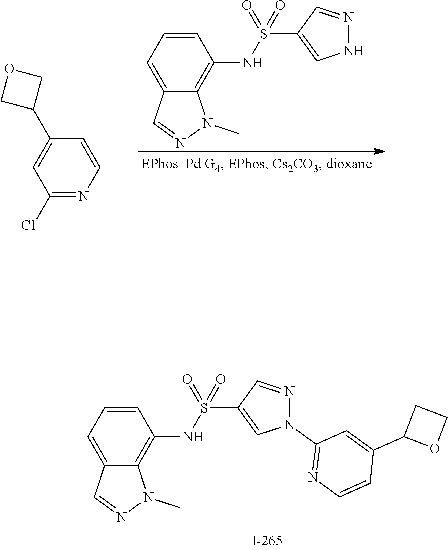 [SMILES: Clc1cc(C2COC2)ccn1 ; Cn1ncc2cccc(NS(=O)(=O)c3cn[nH]c3)c21 ; Cn1ncc2cccc(NS(=O)(=O)c3cnn(-c4cc(C5CCO5)ccn4)c3)c21]

Step 1: Synthesis of N-(1-methylindazol-7-yl)-1-[4-(oxetan-2-yl) pyridin-2-yl]pyrazole-4-sulfonamide (I-265): To a stirred mixture of 2-chloro-4-(oxetan-3-yl) pyridine (73.40 mg, 0.433 mmol, 1.2 equiv) and N-(1-methylindazol-7-yl)-1H-pyrazole-4-sulfonamide (100 mg, 0.361 mmol, 1.00 equiv) in dioxane (5 mL) were added EPhos (19.29 mg, 0.036 mmol, 0.1 equiv), EPhos Pd G4 (33.13 mg, 0.036 mmol, 0.1 equiv) and Cs₂CO₃ (352.49 mg, 1.083 mmol, 3 equiv) in portions at room temperature under nitrogen atmosphere. The resulting mixture was stirred for 2 h at 90° C. under nitrogen atmosphere. The resulting mixture was diluted with water (10 mL). The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% TFA), 30% to 40% gradient in 10 min; detector, UV 254 nm. This resulted in N-(1-methylindazol-7-yl)-1-[4-(oxetan-2-yl) pyridin-2-yl] pyrazole-4-sulfonamide (I-265, 6.9 mg, 4.60%) as a white solid. LC-MS: (ES, m/z): [M+H]⁺=411. ¹H-NMR: (400 MHz, CDCl₃) δ 2.58-2.71 (m, 1H), 3.12-3.24 (m, 1H), 4.42 (s, 3H), 4.68-4.77 (m, 1H), 4.85-4.95 (m, 1H), 5.87 (t, J=7.6 Hz, 1H), 6.36 (s, 1H), 6.77 (d, J=8.0 Hz, 1H), 6.96 (t, J=8.0 Hz, 1H), 7.35 (d, J=5.0 Hz, 1H), 7.71 (d, J=8.0 Hz, 1H), 7.80 (s, 1H), 8.01 (s, 2H), 8.43 (d, J=5.0 Hz, 1H), 8.88 (s, 1H).

### Example 441—Synthesis of 1-{4-[1-Methoxypropyl]Pyridin-2-yl}-N-(1-Methylindazol-7-yl)Pyrazole-4-Sulfonamide (I-297)

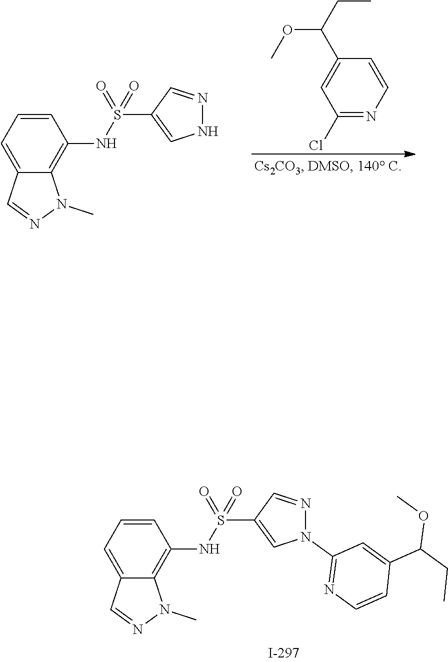 [SMILES: Cn1ncc2cccc(NS(=O)(=O)c3cn[nH]c3)c21 ; CCC(OC)c1ccnc(Cl)c1 ; CCC(OC)c1ccnc(-n2cc(S(=O)(=O)Nc3cccc4cnn(C)c34)cn2)c1]

Step 1: Synthesis of 1-{4-[1-methoxypropyl]pyridin-2-yl}-N-(1-methylindazol-7-yl)pyrazole-4-sulfonamide (I-297): A solution of N-(1-methylindazol-7-yl)-1H-pyrazole-4-sulfonamide (200 mg, 0.721 mmol, 1 equiv), Cs₂CO₃ (704.98 mg, 2.163 mmol, 3 equiv) and 2-chloro-4-(1-methoxypropyl)pyridine (200.85 mg, 1.081 mmol, 1.5 equiv) in DMSO (6 mL) was stirred for overnight at 140° C. under nitrogen atmosphere. The resulting mixture was diluted with water (10 mL). The resulting mixture was extracted with CH₂Cl₂ (3×10 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 20 min; detector, UV 254 nm. The crude product (60 mg) was purified by prep-chiral-HPLC with the following conditions (column: CHIRAKPAKAS3; mobile phase A: hex(0.2% TFA): EtOH=70:30; flow rate: 1 mL/min; gradient: isocratic; injection volume: 3 L) to afford 1-{4-[1-methoxypropyl]pyridin-2-yl}-N-(1-methylindazol-7-yl)pyrazole-4-sulfonamide (I-297, 14.3 mg, 4.60%) as an off-white solid. LC-MS: (ES, m/z): [M+1]=427. ¹H NMR (400 MHz, DMSO-d₆) δ 0.84 (t, J=7.6 Hz, 3H), 1.71 (m, 2H), 3.23 (s, 3H), 4.29 (s, 3H), 4.33 (q, J=6.4 Hz, 1H), 6.72 (d, J=7.2 Hz, 1H), 6.99 (t, J=7.2 Hz, 1H), 7.40 (dd, J=1.6, 5.2 Hz, 1H), 7.71 (d, J=8.0 Hz, 1H), 7.91 (s, 1H), 8.03 (s, 1H), 8.10 (s, 1H), 8.49 (d, J=5.2 Hz, 1H), 8.81 (s, 1H), 10.04 (s, 1H).

### Example 442—Synthesis of 1-(4-(2-Methoxybutan-2-yl)Pyridin-2-yl)-N-(1-Methyl-1H-Indazol-7-yl)-1H-Pyrazole-4-Sulfonamide (I-313)

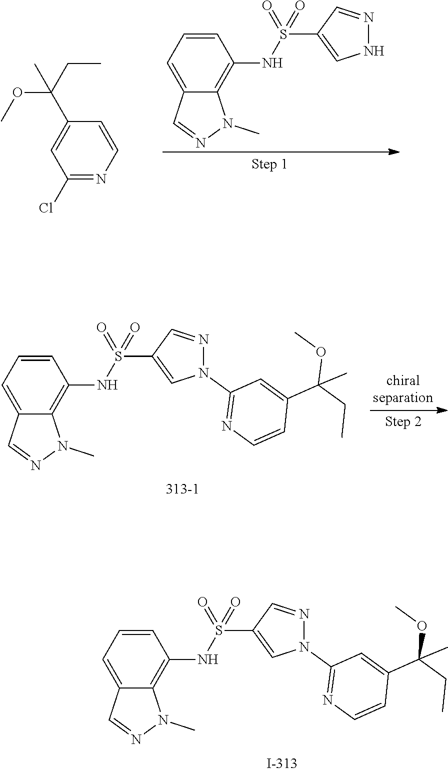 [SMILES: CCC(C)(OC)c1ccnc(Cl)c1 ; Cn1ncc2cccc(NS(=O)(=O)c3cn[nH]c3)c21 ; CCC(C)(OC)c1ccnc(-n2cc(S(=O)(=O)Nc3cccc4cnn(C)c34)cn2)c1 ; CC[C@](C)(OC)c1ccnc(-n2cc(S(=O)(=O)Nc3cccc4cnn(C)c34)cn2)c1]

Step 1: Synthesis of 313-1: A solution of N-(1-methylindazol-7-yl)-1H-pyrazole-4-sulfonamide (100 mg, 0.361 mmol, 1 equiv), Cs₂CO₃ (352.49 mg, 1.083 mmol, 3 equiv) and 2-chloro-4-(2-methoxybutan-2-yl)pyridine (108.01 mg, 0.541 mmol, 1.5 equiv) in DMF (4 mL) was stirred overnight at 140° C. under nitrogen atmosphere. The resulting mixture was diluted with water (10 mL). The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by prep-HPLC with the following conditions (column: XBridge Prep OBD C18 column, 30*150 mm, 5 μm; mobile phase A: water (10 mmol/L NH₄HCO₃+0.05% NH₃·H₂O), mobile phase B: ACN; flow rate: 60 mL/min; gradient: 22% B to 48% B in 7 min; wavelength: 254 nm/220 nm; RT1(min): 8.03; number of runs: 3) to afford 313-1 (50 mg, 33.68%) as a white solid.

Step 2: Synthesis of 1-(4-(2-methoxybutan-2-yl)pyridin-2-yl)-N-(1-methyl-1H-indazol-7-yl)-1H-pyrazole-4-sulfonamide (I-313): The 313-1 (50 mg) was separated by Prep-SFC with the following conditions (column: LuxCellulose-34.6*100 mm, 3 m; mobile phase B: IPA (0.5%2M NH₃-MeOH); flow rate: 4 mL/min; gradient: isocratic) to afford 1-(4-(2-methoxybutan-2-yl)pyridin-2-yl)-N-(1-methyl-1H-indazol-7-yl)-1H-pyrazole-4-sulfonamide (I-313, 16.8 mg, 20.55%) as white solid. LC-MS: (ES, m/z): [M+1]⁺ 441. ¹H NMR (400 MHz, DMSO-d6) δ 0.69 (t, J=7.2 Hz, 3H), 1.50 (s, 3H), 1.73-1.85 (m, 2H), 3.13 (s, 3H), 4.29 (s, 3H), 6.73 (d, J=7.2 Hz, 1H), 6.98 (t, J=7.6 Hz, 1H), 7.44 (dd, J=1.6, 5.2 Hz, 1H), 7.68 (d, J=8.4 Hz, 1H), 7.95 (s, 1H), 8.01 (s, 1H), 8.08 (s, 1H), 8.48 (d, J=5.2 Hz, 1H), 8.80 (s, 1H), 10.02 (s, 1H).

### Example 443—Synthesis of 1-(4-(2-Methoxybutan-2-yl)Pyridin-2-yl)-N-(1-Methyl-1H-Indazol-7-yl)-1H-Pyrazole-4-Sulfonamide (I-314)

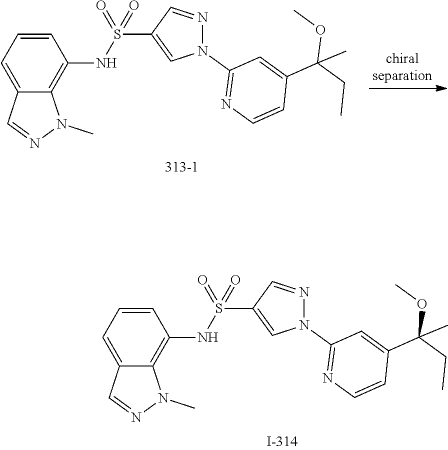 [SMILES: CCC(C)(OC)c1ccnc(-n2cc(S(=O)(=O)Nc3cccc4cnn(C)c34)cn2)c1 ; CC[C@](C)(OC)c1ccnc(-n2cc(S(=O)(=O)Nc3cccc4cnn(C)c34)cn2)c1]

Step 1: Synthesis of 1-(4-(2-methoxybutan-2-yl)pyridin-2-yl)-N-(1-methyl-1H-indazol-7-yl)-1H-pyrazole-4-sulfonamide (I-314): The 313-1 (50 mg) was separated by Prep-SFC with the following conditions (column: LuxCellulose-34.6*100 mm, 3 m; mobile phase B: IPA (0.5%2 M NH₃-MeOH); flow rate: 4 mL/min; gradient: isocratic) to afford 1-(4-(2-methoxybutan-2-yl)pyridin-2-yl)-N-(1-methyl-1H-indazol-7-yl)-1H-pyrazole-4-sulfonamide (I-314, 4.3 mg, 4.64%) as white solid. LC-MS: (ES, m/z): [M+1]⁺ 441. ¹H NMR (400 MHz, DMSO-d₆) δ 0.69 (t, J=7.2 Hz, 3H), 1.50 (s, 3H), 1.73-1.85 (m, 2H), 3.13 (s, 3H), 4.29 (s, 3H), 6.73 (d, J=7.2 Hz, 1H), 6.98 (t, J=7.6 Hz, 1H), 7.44 (dd, J=1.6, 5.2 Hz, 1H), 7.68 (d, J=8.4 Hz, 1H), 7.95 (s, 1H), 8.01 (s, 1H), 8.08 (s, 1H), 8.48 (d, J=5.2 Hz, 1H), 8.80 (s, 1H), 10.02 (s, 1H).

### Example 444—Synthesis of (R)-1-(4-(2-Fluoro-1-Methoxypropan-2-yl)Pyridin-2-yl)-N-(1-Methyl-1H-Indazol-7-yl)-1H-Pyrazole-4-Sulfonamide (I-330) AND (S)-1-(4-(2-Fluoro-1-Methoxypropan-2-yl)Pyridin-2-yl)-N-(1-Methyl-1H-Indazol-7-yl)-1H-Pyrazole-4-Sulfonamide (I-331)

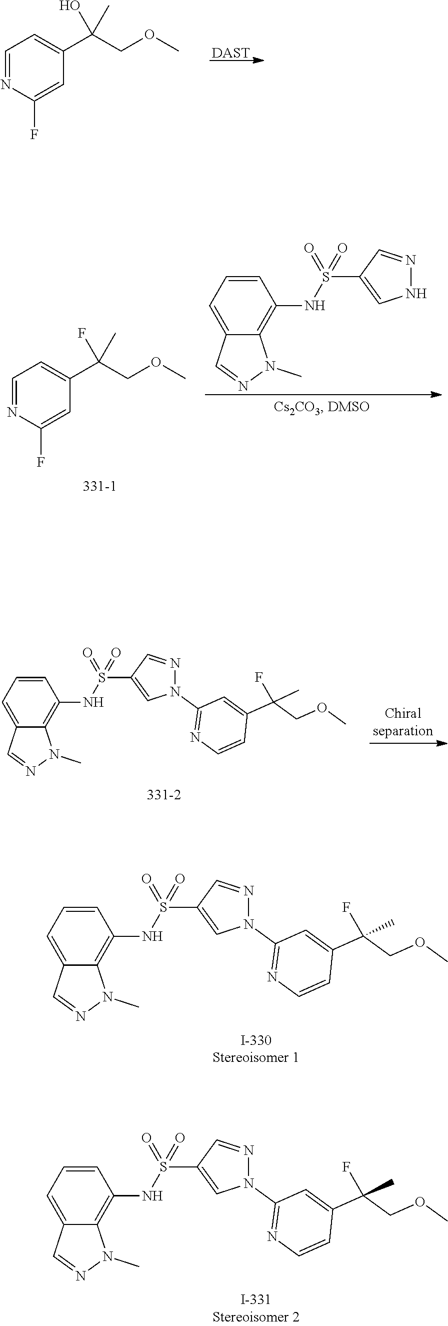

Step 1: Synthesis of 331-1: To a stirred mixture of 2-(2-fluoropyridin-4-yl)-1-methoxypropan-2-ol (200 mg, 1.08 mmol, 1.00 equiv) in DCM (3 mL) was added DAST (353 mg, 2.19 mmol, 2.03 equiv) at 0° C. under nitrogen atmosphere. The resulting mixture was stirred for 2 h at room temperature under nitrogen atmosphere. The reaction was quenched with sat. NH₄Cl (aq.) at 0° C. The resulting mixture was extracted with CH₂Cl₂ (3×3 mL). The combined organic layers were washed with brine (1×5 mL), dried over anhydrous Na₂SO₄. After filtration, the filtrate was concentrated under reduced pressure. The crude product was used in the next step directly without further purification. LC-MS: (ES, m/z): [M+H]=188

Step 2: Synthesis of 331-2: A mixture of 2-fluoro-4-(2-fluoro-1-methoxypropan-2-yl) pyridine (150 mg, 0.801 mmol, 1.00 equiv), N-(1-methylindazol-7-yl)-1H-pyrazole-4-sulfonamide (222 mg, 0.801 mmol, 1.00 equiv) and Cs₂CO₃ (783.26 mg, 2.40 mmol, 3.00 equiv) in DMSO (5 mL) was stirred for overnight at 100° C. under nitrogen atmosphere. The reaction was quenched with water at room temperature. The resulting mixture was extracted with EtOAc (3×3 mL). The combined organic layers were washed with brine (1×5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. This resulted in 1-[4-(2-fluoro-1-methoxypropan-2-yl)pyridin-2-yl]-N-(1-methylindazol-7-yl)pyrazole-4-sulfonamide (331-2, 150 mg, 52%) as a white solid. LC-MS: (ES, m/z): [M+H]=455.

Step 3: Chiral separation of (R)-1-(4-(2-fluoro-1-methoxypropan-2-yl)pyridin-2-yl)-N-(1-methyl-1H-indazol-7-yl)-1H-pyrazole-4-sulfonamide (I-330) and (S)-1-(4-(2-fluoro-1-methoxypropan-2-yl)pyridin-2-yl)-N-(1-methyl-1H-indazol-7-yl)-1H-pyrazole-4-sulfonamide (I-331): 331-2 (150 mg, 0.341 mmol, 1 equiv) was purified by Prep-SFC with the following conditions (column: CHIRALPAKIG3; mobile phase A: hex (0.2% TFA): (MeOH: DCM=1:1)=75:25; flow rate: 1 mL/min; gradient: isocratic; injection volume: 0.2 mL) to afford (R)-1-(4-(2-fluoro-1-methoxypropan-2-yl)pyridin-2-yl)-N-(1-methyl-1H-indazol-7-yl)-1H-pyrazole-4-sulfonamide (I-330, 77.1 mg) as a white solid and (S)-1-(4-(2-fluoro-1-methoxypropan-2-yl)pyridin-2-yl)-N-(1-methyl-1H-indazol-7-yl)-1H-pyrazole-4-sulfonamide (I-331, 81.1 mg) as a white solid. I-330: LC-MS: (ES, m/z): [M+H]⁺=445. ¹H NMR (400 MHz, methanol-d₄) δ1.71 (d, J=22.0 Hz, 3H), 3.38 (s, 3H), 3.62-3.82 (m, 2H), 4.34 (s, 3H), 6.72 (dd, J=7.4, 1.0 Hz, 1H), 6.94 (dd, J=8.4, 7.4 Hz, 1H), 7.39 (dd, J=5.2, 1.6 Hz, 1H), 7.73 (dd, J=8.4, 1.0 Hz, 1H), 7.87 (s, 1H), 7.98-8.05 (m, 2H), 8.34 (d, J=5.2 Hz, 1H), 8.83 (d, J=1.2 Hz, 1H). I-331: LC-MS: (ES, m/z): [M+H]⁺=445. ¹H NMR (400 MHz, methanol-d₄) δ1.69 (d, J=22.4 Hz, 3H), 3.37 (s, 3H), 3.66-3.84 (m, 2H), 4.37 (s, 3H), 6.73 (dd, J=7.3, 0.9 Hz, 1H), 6.97 (t, J=7.7 Hz, 1H), 7.41 (dd, J=5.3, 1.6 Hz, 1H), 7.71 (d, J=8.0 Hz, 1H), 7.86 (s, 1H), 8.08-7.99 (m, 2H), 8.44 (d, J=5.2 Hz, 1H), 8.79 (s, 1H).

### Example 445—Synthesis of (R)-1-(4-(3,4-Dimethyl-2-OXOIMIDAZOLIDIN-1-yl)Pyridin-2-yl)-N-(1-Methyl-1H-Indazol-7-yl)-1H-Pyrazole-4-Sulfonamide (I-347)

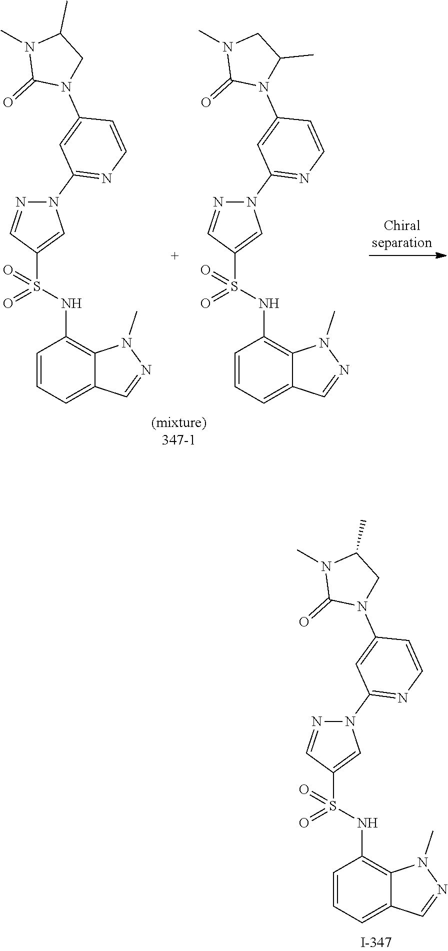 [SMILES: CC1CN(c2ccnc(-n3cc(S(=O)(=O)Nc4cccc5cnn(C)c45)cn3)c2)C(=O)N1C ; CC1CN(C)C(=O)N1c1ccnc(-n2cc(S(=O)(=O)Nc3cccc4cnn(C)c34)cn2)c1 ; C[C@@H]1CN(c2ccnc(-n3cc(S(=O)(=O)Nc4cccc5cnn(C)c45)cn3)c2)C(=O)N1C]

Step 1: Synthesis of (R)-1-(4-(3,4-dimethyl-2-oxoimidazolidin-1-yl)pyridin-2-yl)-N-(1-methyl-1H-indazol-7-yl)-1H-pyrazole-4-sulfonamide (I-347): 347-1 (mixture, 400 mg) was purified by prep-chiral-HPLC with the following conditions (column: CHIRAL ART Cellulose-SB, 2*25 cm, 5 μm; mobile phase A: hex (0.2% TFA), mobile phase B: ETOH:DCM=1:1; flow rate: 20 mL/min; gradient: 25% B to 25% B in 25 min; wavelength: 220/254 nm; RT1(min): 14.64; RT2(min): 17.56; sample solvent: EtOH:DCM=1:1; injection volume: 0.4 mL; number of runs: 35) to afford (R)-1-(4-(3,4-dimethyl-2-oxoimidazolidin-1-yl)pyridin-2-yl)-N-(1-methyl-1H-indazol-7-yl)-1H-pyrazole-4-sulfonamide (I-347, 40.8 mg, 10.2%) as an off-white solid. LC-MS: (ES, m/z): [M+H]⁺=467. ¹H-NMR: (400 MHz, DMSO, ppm) δ 1.28 (d, J=6.2 Hz, 3H), 2.78 (s, 3H), 3.44 (m, J=9.6, 6.8 Hz, 1H), 3.77 (m, J=14.8, 6.4 Hz, 1H), 4.08 (t, J=9.2 Hz, 1H), 4.29 (s, 3H), 6.71 (m, J=7.8, 1.0 Hz, 1H), 6.96 (t, J=7.8 Hz, 1H), 7.50 (m, J=5.8, 2.2 Hz, 1H), 7.62 (s, 1H), 7.99 (s, 1H), 8.06 (s, 1H), 8.25 (d, J=2.2 Hz, 1H), 8.31 (d, J=5.8 Hz, 1H), 8.74 (s, 1H), 10.07 (s, 1H).

### Example 446—Synthesis of 1-[4-(3-Fluorooxetan-3-yl)Pyridin-2-yl]-N-(3-Methyl-1H-Indazol-4-yl)Pyrazole-4-Sulfonamide (I-240)

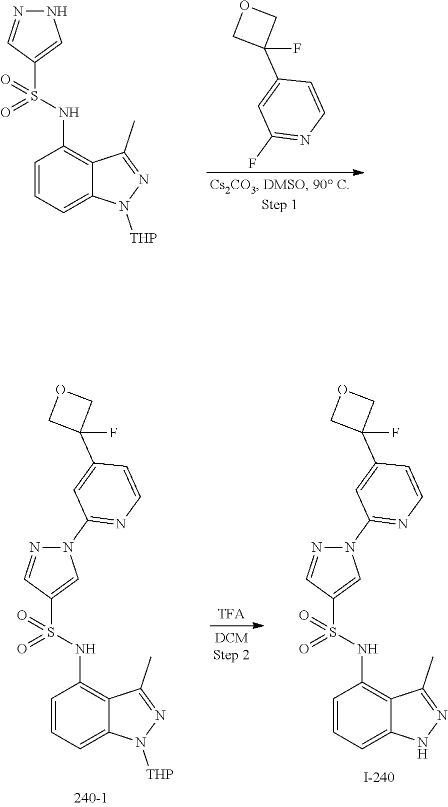

Step 1: Synthesis of 240-1: A solution of N-[3-methyl-1-(oxan-2-yl)indazol-4-yl]-1H-pyrazole-4-sulfonamide (120 mg, 0.332 mmol, 1 equiv), Cs₂CO₃ (324.54 mg, 0.996 mmol, 3 equiv) and 2-fluoro-4-(3-fluorooxetan-3-yl)pyridine (68.19 mg, 0.398 mmol, 1.2 equiv) in DMSO (6 mL) was stirred for overnight at 90° C. under nitrogen atmosphere. The resulting mixture was diluted with water (10 mL) and extracted with CH₂Cl₂ (3×10 mL). The combined organic layers were washed with water (1×30 mL), dried over anhydrous Na₂SO₄. After filtration, the filtrate was concentrated under reduced pressure. The crude product was used in the next step directly without further purification. This resulted in 1-(4-(3-fluorooxetan-3-yl)pyridin-2-yl)-N-(3-methyl-1-(tetrahydro-2H-pyran-2-yl)-1H-indazol-4-yl)-1H-pyrazole-4-sulfonamide (240-1, 200 mg, 76.39%) as a yellow oil.

Step 2: Synthesis of 1-[4-(3-fluorooxetan-3-yl)pyridin-2-yl]-N-(3-methyl-1H-indazol-4-yl)pyrazole-4-sulfonamide (I-240): A solution of 1-(4-(3-fluorooxetan-3-yl)pyridin-2-yl)-N-(3-methyl-1-(tetrahydro-2H-pyran-2-yl)-1H-indazol-4-yl)-1H-pyrazole-4-sulfonamide (150 mg, 0.293 mmol, 1 equiv) and TFA (3 mL) in DCM (1.5 mL) was stirred for 3 h at room temperature under nitrogen atmosphere. The resulting mixture was diluted with water (10 mL) and extracted with CH₂Cl₂ (3×10 mL). The combined organic layers were washed with water (1×30 mL), dried over anhydrous Na₂SO₄. After filtration, the filtrate was concentrated under reduced pressure. The crude product (120 mg) was purified by prep-HPLC with the following conditions (column: Xbridge Prep OBD C18 column, 30*150 mm, 5 μm; mobile phase A: water(10 mmol/L NH₄HCO₃+0.05% NH₃H₂O), mobile phase B: ACN; flow rate: 60 mL/min; gradient: 10% B to 30% B in 7 min; wavelength: 254 nm/220 nm; RT1(min): 7; number of runs: 3) to afford 1-[4-(3-fluorooxetan-3-yl)pyridin-2-yl]-N-(3-methyl-1H-indazol-4-yl)pyrazole-4-sulfonamide (I-240, 53.5 mg, 42.63%) as a off-white solid. LC-MS: (ES, m/z): [M+1]=429. ¹H-NMR: ¹H NMR (400 MHz, Methanol-d₄) δ 2.69 (s, 3H), 4.91-4.93 (m, 2H), 5.08-5.14 (m, 2H), 6.67 (d, J=7.2 Hz, 1H), 7.19 (dd, J=7.2, 8.2 Hz, 1H), 7.30 (d, J=8.2 Hz, 1H), 7.61 (dd, J=1.6, 5.2 Hz, 1H), 7.87 (s, 1H), 8.18 (d, J=1.6 Hz, 1H), 8.53 (d, J=5.2 Hz, 1H), 8.81 (s, 1H).

### Example 447—Synthesis of 1-(4-(3-Methyoxyoxetan-3-yl)Pyridin-2-yl)-N-(3-Methyl-1H-Indazol-4-yl)-1H-Pyrazole-4-Sulfonamide (I-244

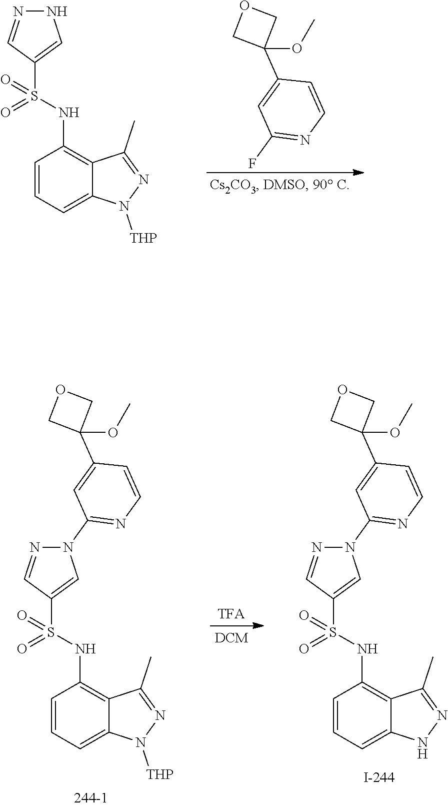

Step 1: Synthesis 244-1: A solution of N-[3-methyl-1-(oxan-2-yl)indazol-4-yl]-1H-pyrazole-4-sulfonamide (100 mg, 0.277 mmol, 1 equiv), Cs₂CO₃ (270.45 mg, 0.831 mmol, 3 equiv) and 2-fluoro-4-(3-methoxyoxetan-3-yl)pyridine (60.82 mg, 0.332 mmol, 1.2 equiv) in DMSO (5 mL) was stirred for overnight at 90° C. under nitrogen atmosphere. The resulting mixture was diluted with water (10 mL) and extracted with CH₂Cl₂ (3×10 mL). The combined organic layers were washed with brine (1×30 mL), dried over anhydrous Na₂SO₄. After filtration, the filtrate was concentrated under reduced pressure. The crude product mixture was used in the next step directly without further purification. This resulted in 244-1 (150 mg, 82.67%) as a yellow oil.

Step 2: Synthesis of 1-(4-(3-methoxyoxetan-3-yl)pyridin-2-yl)-N-(3-methyl-1H-indazol-4-yl)-1H-pyrazole-4-sulfonamide (I-244): A solution of 244-1 (100 mg, 0.191 mmol, 1 equiv) and TFA (3 mL) in DCM (1.5 mL) was stirred for 3 h at room temperature under nitrogen atmosphere. The resulting mixture was diluted with water (10 mL) and extracted with CH₂Cl₂ (3×10 mL). The combined organic layers were washed with brine (1×30 mL), dried over anhydrous Na₂SO₄. After filtration, the filtrate was concentrated under reduced pressure. The crude product (110 mg) was purified by prep-HPLC with the following conditions (column: Xbridge Prep OBD C18 column, 30*150 mm, 5 μm; mobile phase A: water(10 mmol/L NH₄HCO₃)+0.05% NH₃·H₂O, mobile phase B: ACN; flow rate: 60 mL/min; gradient: 10% B to 32% B in 7 min; wavelength: 254 nm/220 nm; RT1(min): 8.3) to afford 1-(4-(3-methoxyoxetan-3-yl)pyridin-2-yl)-N-(3-methyl-1H-indazol-4-yl)-1H-pyrazole-4-sulfonamide (I-244, 42.3 mg, 50.23%) as a white solid. LC-MS: (ES, m/z): [M+H]⁺=441. ¹H NMR (400 MHz, DMSO-d₆) δ 2.60 (s, 3H), 3.18 (s, 3H), 4.73 (d, J=7.2 Hz, 2H), 4.87 (d, J=7.2 Hz, 2H), 6.57 (d, J=7.2 Hz, 1H), 7.17 (t, J=7.6 Hz, 1H), 7.32 (d, J=8.2 Hz, 1H), 7.61 (dd, J=1.6, 5.2 Hz, 1H), 8.02 (s, 1H), 8.03 (s, 1H), 8.59 (d, J=5.2 Hz, 1H), 8.82 (s, 1H), 9.92 (s, 1H), 12.71 (s, 1H).

### Example 448—Synthesis of N-(1-Methyl-1H-Indazol-7-yl)-1-(4-(3-Methylazetidin-1-yl)Pyridin-2-yl)-1H-Pyrazole-4-Sulfonamide (I-356)

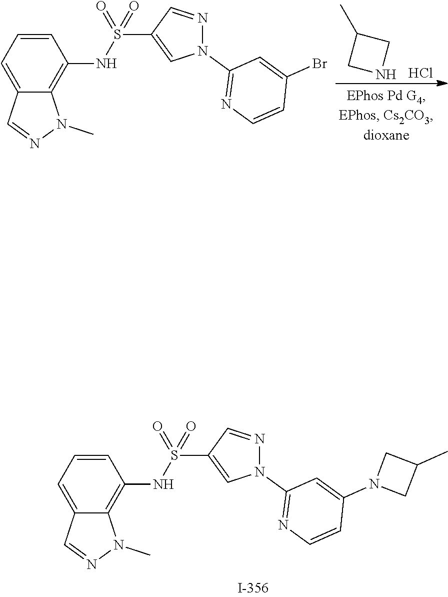

To a stirred solution of 1-(4-bromopyridin-2-yl)-N-(1-methylindazol-7-yl) pyrazole-4-sulfonamide (100 mg, 0.231 mmol, 1 equiv) and 3-methylazetidin-1-ium chloride (49.66 mg, 0.462 mmol, 2 equiv) in 1,4-dioxane (2 mL) were added Ephos (12.34 mg, 0.023 mmol, 0.1 equiv), Cs₂CO₃ (225.59 mg, 0.693 mmol, 3 equiv) and Ephos Pd G4 (21.20 mg, 0.023 mmol, 0.1 equiv) in portions at room temperature under a nitrogen atmosphere. The resulting mixture was stirred for 2 h at 100° C. under a nitrogen atmosphere. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 50% gradient in 12 min; detector: UV 254 nm. The resulting mixture was concentrated under reduced pressure. This resulted in N-(1-methyl-1H-indazol-7-yl)-1-(4-(3-methylazetidin-1-yl)pyridin-2-yl)-1H-pyrazole-4-sulfonamide (I-356, 31.7 mg, 32.40%) as an off-white solid. LC-MS (ES, m/z): [M+H]=424; 1H NMR (400 MHz, DMSO-d₆): δ 1.26 (d, J=6.8 Hz, 3H), 2.83-2.92 (m, 1H), 3.60 (dd, J=8.2, 8.0 Hz, 2H), 4.15 (d, J=8.0 Hz, 2H), 4.27 (s, 3H), 6.39 (dd, J=4.0, 2.2 Hz, 1H), 6.69 (d, J=7.2 Hz, 1H), 6.84 (d, J=2.0 Hz, 1H), 6.98 (t, J=8.0 Hz, 1H), 7.70 (dd, J=4.0, 1.0 Hz, 1H), 7.96 (s, 1H), 8.01 (d, J=8.0 Hz, 1H), 8.09 (s, 1H), 8.76 (s, 1H), 9.99 (s, 1H).

### Example 449—Synthesis of 1-(4-((1-Methoxy-3-Methylcyclobutyl)Pyridin-2-yl)-N-(1-Methyl-1H-Indazol-7-yl)-1H-Pyrazole-4-Sulfonamide (I-362)

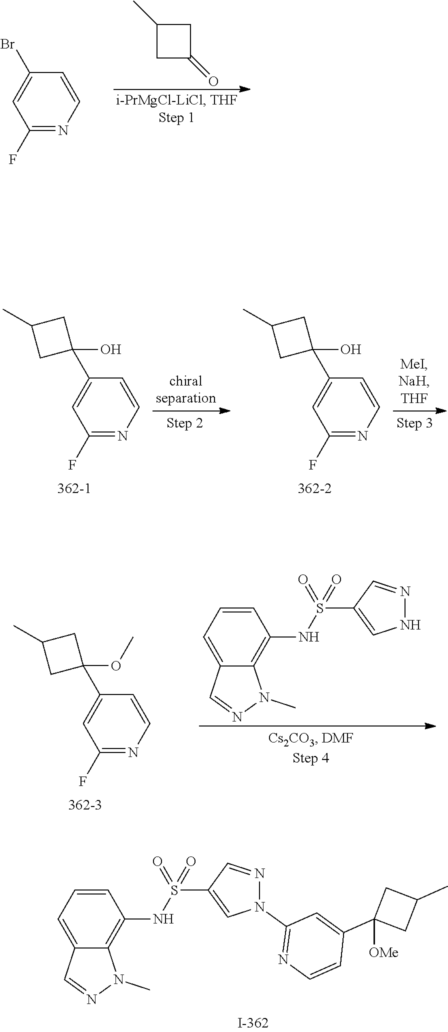

Step 1: Synthesis of 362-1: A solution of 4-bromo-2-fluoropyridine (2 g, 11.364 mmol, 1 equiv) in THE (50 mL) was treated with isopropylmagnesium chloride lithium chloride (2.48 g, 17.046 mmol, 1.5 equiv) for 30 min at −78° C. under a nitrogen atmosphere followed by the addition of 3-methylcyclobutan-1-one (2.10 g, 25.001 mmol, 2.20 equiv) dropwise at −60° C. Then the mixture was stirred from −60° C. to room temperature overnight. After the reaction was completed, the reaction was quenched with sat. NH₄Cl (aq.) at room temperature, the aqueous layer was extracted with EtOAc (3×100 mL), washed with brine (1×100 mL), and dried over anhydrous Na₂SO₄ to give the crude. The residue was purified by prep-TLC (PE/EA 4:1) to afford 1-(2-fluoropyridin-4-yl)-3-methylcyclobutan-1-ol (362-1, 1.632 g, 73.70%) as a yellow oil. (ES, m/z): [M+H]⁺=182.

Step 2: Synthesis of 362-2: The racemic product (362-1, 2.2 g) was separated by chiral-HPLC with the following conditions (column: CHIRALPAK IG, 4.6*250 mm, Sum; mobile phase A: CO₂, mobile phase B: MeOH; flow rate: 100 mL/min; gradient: isocratic 20% B; column temperature (° C.): 35; back pressure(bar): 100; wavelength: 220 nm; RT1(min): 6.68; RT2(min): 7.68; sample solvent: MeOH; injection volume: 2.5 mL; number of runs: 20) to afford 1-(2-fluoropyridin-4-yl)-3-methylcyclobutan-1-ol (362-2, 238 mg) as an off-white solid.

Step 3: Synthesis of 362-3: To a solution of 1-(2-fluoropyridin-4-yl)-3-methylcyclobutan-1-ol (228 mg, 1.258 mmol, 1 equiv) in THE (4 mL) was added NaH (90.58 mg, 3.774 mmol, 3 equiv) at 0° C. The mixture was stirred for 15 min. Mel (535.77 mg, 3.774 mmol, 3 equiv) was added and the mixture was allowed to warm to rt and stirred for 1 h. The reaction mixture was quenched by water (20 mL) and extracted with DCM (3×25 mL). The resulting mixture was concentrated under reduced pressure. This resulted in 2-fluoro-4-((1r,3r)-1-methoxy-3-methylcyclobutyl)pyridine (362-3, 211 mg, 65.97%) as a yellow oil.

Step 4: Synthesis of 1-(4-((1-methoxy-3-methylcyclobutyl)pyridin-2-yl)-N-(1-methyl-1H-indazol-7-yl)-1H-pyrazole-4-sulfonamide (I-362): A solution of 2-fluoro-4-((1r,3r)-1-methoxy-3-methylcyclobutyl)pyridine (100 mg, 0.512 mmol, 1 equiv), N-(1-methylindazol-7-yl)-1H-pyrazole-4-sulfonamide (170.44 mg, 0.614 mmol, 1.2 equiv) and Cs₂CO₃ (500.65 mg, 1.536 mmol, 3 equiv) in DMF (3 mL) was stirred overnight at 100° C. The mixture was allowed to cool down to room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions (column: RP Prep OBD C18 column, 30*150 mm, 5 μm; mobile phase A: water (10 mmol/L NH₄HCO₃⁺0.05% NH₃H₂O), mobile phase B: ACN; flow rate: 60 mL/min; gradient: 18% B to 48% B in 8 min; wavelength: 220 nm; RT1(min): 9.48) to afford 1-(4-(1-methoxy-3-methylcyclobutyl)pyridin-2-yl)-N-(1-methyl-1H-indazol-7-yl)-1H-pyrazole-4-sulfonamide as a single steroeoisomer (I-362, 64.1 mg, 27.18%) as an orange solid. LC-MS (ES, m/z): [M+H]⁺=453; ¹H NMR (400 MHz, methanol-d₄): δ 1.13 (d, J=6.0 Hz, 3H), 1.98-2.04 (m, 2H), 2.57-2.66 (m, 3H), 3.06 (s, 3H), 4.38 (s, 3H), 6.74 (dd, J=1.2, 7.2 Hz, 1H), 6.97 (t, J=7.8 Hz, 1H), 7.37 (dd, J=1.2, 5.2 Hz, 1H), 7.70 (dd, J=1.2, 8.2 Hz, 1H), 7.86 (d, J=0.8 Hz, 1H), 7.96 (s, 1H), 8.02 (s, 1H), 8.44 (d, J=5.0 Hz, 1H), 8.79 (d, J=0.8 Hz, 1H).

### Example 450—Synthesis of 1-(4-(1-Methoxy-3-Methyl-Cyclobutyl)Pyridin-2-yl)-N-(1-Methyl-1H-Indazol-7-yl)-1H-Pyrazole-4-Sulfonamide (I-363)

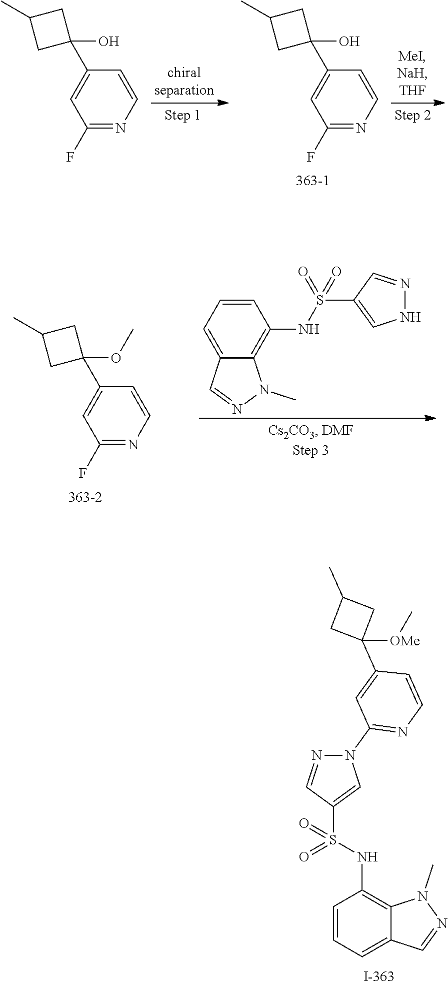

Step 1: Synthesis of 363-1: The racemic product (2.2 g) was purified by chiral-HPLC with the following conditions (column: CHIRALPAK IG, 4.6*250 mm, 5 um; mobile phase A: CO₂, mobile phase B: MeOH; flow rate: 100 mL/min; gradient: isocratic 20% B; column temperature (° C.): 35; back pressure(bar): 100; wavelength: 220 nm; RT1(min): 6.68; RT2(min): 7.68; sample solvent: MeOH; injection volume: 2.5 mL; number of runs: 20) to afford (1r,3s)-1-(2-fluoropyridin-4-yl)-3-methylcyclobutan-1-ol (363-1, 1.523 g) as an off-white oil.

Step 2: Synthesis of 363-2: To a solution of 1-(2-fluoropyridin-4-yl)-3-methylcyclobutan-1-ol (400 mg, 2.207 mmol, 1 equiv) in THF(1 mL) was added NaH (158.92 mg, 6.621 mmol, 3 equiv) at 0° C. The mixture was stirred for 15 min. Mel (939.94 mg, 6.621 mmol, 3 equiv) was added and the mixture was allowed to warm to rt and stirred for 1 h. The reaction mixture was quenched with water (20 mL) and extracted with DCM (3×25 mL). The resulting mixture was concentrated under reduced pressure. This resulted in 2-fluoro-4-[(1r,3s)-1-methoxy-3-methylcyclobutyl]pyridine (363-2, 386 mg, 70.22%) as a yellow oil.

Step 3: Synthesis of 1-(4-(1-methoxy-3-methylcyclobutyl)pyridin-2-yl)-N-(1-methyl-1H-indazol-7-yl)-1H-pyrazole-4-sulfonamide (I-363): A solution of 2-fluoro-4-[1-methoxy-3-methylcyclobutyl]pyridine (100 mg, 0.512 mmol, 1 equiv), N-(1-methylindazol-7-yl)-1H-pyrazole-4-sulfonamide (170.44 mg, 0.614 mmol, 1.2 equiv) and Cs₂CO₃ (500.65 mg, 1.536 mmol, 3 equiv) in DMF (3 mL) was stirred overnight at 100° C. The mixture was allowed to cool down to room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions (column: XBridge Prep OBD C18 column: 30*150 mm, 5 μm; mobile phase A: water (10 mmol/L NH₄HCO₃+0.05% NH₃·H₂O), mobile phase B: ACN; flow rate: 60 mL/min; gradient: 21% B to 48% B in 7 min; wavelength: 254 nm/220 nm; RT1(min): 8.67) to afford N-(1-methylindazol-7-yl)-1-{4-[1-methoxy-3-methylcyclobutyl]pyridin-2-yl}pyrazole-4-sulfonamide as a single steroeoisomer (I-363, 80.8 mg, 34.69%) as an off-white solid. LCMS (ES, m/z): [M+H]⁺=453; ¹H NMR (400 MHz, methanol-d₄): δ 1.21 (d, J=6.4 Hz, 3H), 1.98-2.14 (m, 3H), 2.56-2.65 (m, 2H), 3.01 (s, 3H), 4.38 (s, 3H), 6.75 (d, J=7.2 Hz, 1H), 6.97 (t, J=7.6 Hz, 1H), 7.49 (dd, J=5.2, 1.6 Hz, 1H), 7.69 (d, J=8.0 Hz, 1H), 7.86 (s, 1H), 8.02 (s, 1H), 8.09 (d, J=1.6 Hz, 1H), 8.46 (d, J=5.2 Hz, 1H), 8.80 (s, 1H).

### Example 451—Synthesis of 1-(4-(1,3-Difluorocyclobutyl) Pyridin-2-yl)-N-(1-Methyl-1H-Indazol-7-yl)-1H-Pyrazole-4-Sulfonamide (I-366)

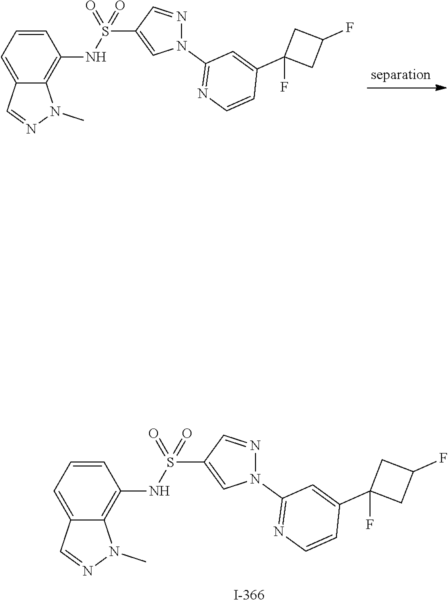 [SMILES: Cn1ncc2cccc(NS(=O)(=O)c3cnn(-c4cc(C5(F)CC(F)C5)ccn4)c3)c21 ; Cn1ncc2cccc(NS(=O)(=O)c3cnn(-c4cc(C5(F)CC(F)C5)ccn4)c3)c21]

1-(4-(1,3-difluorocyclobutyl)pyridin-2-yl)-N-(1-methyl-1H-indazol-7-yl)-1H-pyrazole-4-sulfonamide (100 mg) was purified by prep-HPLC with the following conditions (column: XBridge Prep OBD C18 column: 30*150 mm, 5 μm; mobile phase A: water (10 mmol/L NH₄HCO₃+0.05% NH3·H2O), mobile phase B: ACN; flow rate: 60 mL/min; gradient: 25% B to 47% B in 7 min; wavelength: 254 nm/220 nm; RT1(min): 7.72; number of runs: 2) to afford 1-(4-(1,3-difluorocyclobutyl)pyridin-2-yl)-N-(1-methyl-1H-indazol-7-yl)-1H-pyrazole-4-sulfonamide (I-366, 9.4 mg, 9.4%) as an off-white solid. LC-MS (ES, m/z): [M+H]⁺=445; ¹H NMR (400 MHz, DMSO-d₆): δ 2.88 (m, J=24.8, 15.2, 5.2 Hz, 2H), 3.10 (m, J=15.2, 12.2, 6.8 Hz, 2H), 4.22 (s, 3H), 5.19 (m, J=55.6, 12.2, 6.8, 5.2 Hz, 1H), 6.65 (m, J=7.4, 1.0 Hz, 1H), 6.89 (t, J=7.8 Hz, 1H), 7.45 (m, J=5.4, 1.6 Hz, 1H), 7.59 (d, J=8.0 Hz, 1H), 7.87 (d, J=1.6 Hz, 1H), 7.96-8.02 (m, 2H), 8.51 (d, J=5.2 Hz, 1H), 8.75 (d, J=0.8 Hz, 1H), 10.00 (s, 1H).

### Example 452—Synthesis of 1-(4-(3,3-Dimethylazetidin-1-yl)Pyridin-2-yl)-N-(6-Methoxy-1-Methyl-1H-Indazol-7-yl)-1H-Pyrazole-4-Sulfonamide (I-370)

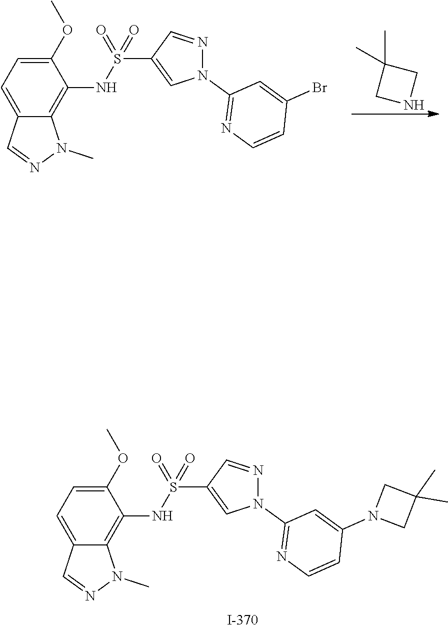

A solution of 1-(4-bromopyridin-2-yl)-N-(6-methoxy-1-methylindazol-7-yl) pyrazole-4-sulfonamide (200 mg, 0.432 mmol, 1 equiv) and 3,3-dimethylazetidine (74 mg, 0.869 mmol, 2.01 equiv), EPhos (23.00 mg, 0.043 mmol, 0.10 equiv), EPhos Pd G₄ (40 mg, 0.044 mmol, 0.10 equiv) and Cs₂CO₃ (420 mg, 1.289 mmol, 2.99 equiv) in 1,4-dioxane (5 mL) was stirred for 2 h at 100° C. under a nitrogen atmosphere. The resulting mixture was diluted with water (5 mL). The resulting mixture was extracted with EtOAc (3×20 mL). The combined organic layers were washed with brine (1×3 mL) and dried over anhydrous Na₂SO₄. After filtration, the filtrate was concentrated under reduced pressure. The crude product (60 mg) was purified by prep-HPLC with the following conditions (column: XBridge Prep OBD C18 column: 30*150 mm, 5 μm; mobile phase A: water(10 mmol/L NH₄HCO₃+0.05% NH₃·H₂O), mobile phase B: ACN; flow rate: 60 mL/min; gradient: 40% B to 60% B in 7 min; wavelength: 254 nm/220 nm; RT1(min): 7.68) to afford 1-[4-(3,3-dimethylazetidin-1-yl)pyridin-2-yl]-N-(6-methoxy-1-methylindazol-7-yl)pyrazole-4-sulfonamide as a single steroeoisomer (1-370, 16.7 mg, 8.27%) as an off-white solid. LC-MS (ES, m/z): [M+1]*=468; ¹H NMR 1H NMR (400 MHz, chloroform-d): 61.36 (s, 6H), 3.40 (dt, J=11.2, 4.8 Hz, 3H), 3.67-3.76 (m, 4H), 4.41 (dt, J=11.0, 5.2 Hz, 3H), 6.18 (s, 1H), 6.45 (s, 1H), 6.62-6.70 (m, 1H), 6.78-6.84 (m, 1H), 7.60-7.67 (m, 2H), 7.89-8.01 (m, 2H), 8.71 (s, 1H).

### Example 453—Synthesis of 1-(4-(5-Azaspiro[2.3]Hexan-5-yl)Pyridin-2-yl)-N-(6-Methoxy-1-Methyl-1H-Indazol-7-yl)-1H-Pyrazole-4-Sulfonamide (I-371)

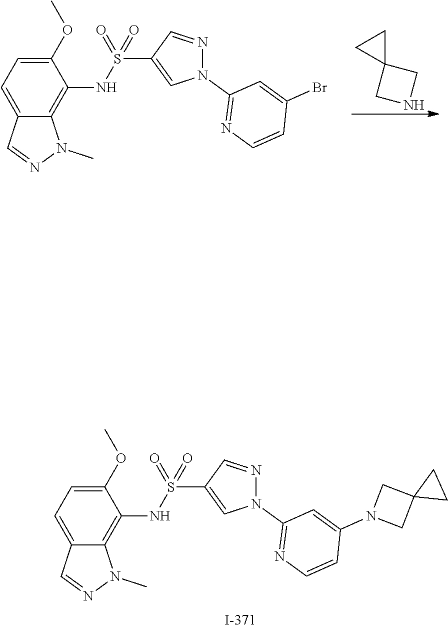 [SMILES: COc1ccc2cnn(C)c2c1NS(=O)(=O)c1cnn(-c2cc(Br)ccn2)c1 ; C1CC12CNC2 ; COc1ccc2cnn(C)c2c1NS(=O)(=O)c1cnn(-c2cc(N3CC4(CC4)C3)ccn2)c1]

To a stirred mixture of 1-(4-bromopyridin-2-yl)-N-(6-methoxy-1-methylindazol-7-yl)pyrazole-4-sulfonamide (180 mg, 0.389 mmol, 1 equiv) and Cs₂CO₃ (379.75 mg, 1.167 mmol, 3 equiv) in dioxane (10 mL) were added EPhos (20.78 mg, 0.039 mmol, 0.1 equiv), 5-azaspiro[2.3]hexane (64.60 mg, 0.778 mmol, 2 equiv) and EPhos Pd G4 (35.69 mg, 0.039 mmol, 0.1 equiv) dropwise at 100° C. under a nitrogen atmosphere. The resulting mixture was stirred for an additional 2 h at 100° C. The crude product (180 mg) was purified by prep-HPLC with the following conditions (column: XBridge Prep OBD C18 column: 30*150 mm, 5 μm; mobile phase A: water (10 mmol/L NH₄HCO₃+0.05% NH₃·H₂O), mobile phase B: ACN; flow rate: 60 mL/min; gradient: 33% B to 60% B in 7 min; wavelength: 254 nm/220 nm; RT1(min): 7.8) to 1-(4-(5-azaspiro[2.3]hexan-5-yl)pyridin-2-yl)-N-(6-methoxy-1-methyl-1H-indazol-7-yl)-1H-pyrazole-4-sulfonamide (I-371, 11.1 mg, 6.09%) as a light yellow solid. LC-MS (ES, m/z): [M+1]=466; ¹H NMR (400 MHz, DMSO-d₆): δ0.72 (s, 4H), 3.33 (s, 3H), 4.09 (s, 4H), 4.24 (s, 3H), 6.41 (dd, J=5.8, 2.2 Hz, 1H), 6.75-6.95 (m, 2H), 7.67 (d, J=8.8 Hz, 1H), 7.89 (s, 1H), 7.98 (s, 1H), 8.04 (dd, J=5.8, 1.2 Hz, 1H), 8.63 (s, 1H).

### Example 454—Synthesis of 1-(4-(1,3-Difluorocyclobutyl) Pyridin-2-yl)-N-(1-Methyl-1H-Indazol-7-yl)-1H-Pyrazole-4-Sulfonamide (I-376)

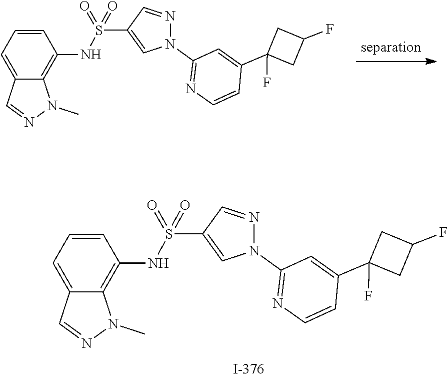 [SMILES: Cn1ncc2cccc(NS(=O)(=O)c3cnn(-c4cc(C5(F)CC(F)C5)ccn4)c3)c21 ; Cn1ncc2cccc(NS(=O)(=O)c3cnn(-c4cc(C5(F)CC(F)C5)ccn4)c3)c21]

1-(4-(1,3-difluorocyclobutyl)pyridin-2-yl)-N-(1-methyl-1H-indazol-7-yl)-1H-pyrazole-4-sulfonamide (100 mg) was purified by prep-HPLC with the following conditions (column: XBridge Prep OBD C18 column: 30*150 mm, 5 μm; mobile phase A: water (10 mmol/L NH₄HCO₃+0.05% NH₃·H₂O), mobile phase B: ACN; flow rate: 60 mL/min; gradient: 25% B to 47% B in 7 min; wavelength: 254 nm/220 nm; RT1(min): 7.72; number of runs: 2) to afford 1-(4-(1,3-difluorocyclobutyl)pyridin-2-yl)-N-(1-methyl-1H-indazol-7-yl)-1H-pyrazole-4-sulfonamide as a single steroeoisomer (I-376, 1.7 mg, 1.7%) as an off-white solid. LC-MS (ES, m/z): [M+H]⁺=445; ¹H NMR (400 MHz, DMSO-d₆): δ 2.91 (m, J=20.4, 5.4 Hz, 2H), 3.03-3.14 (m, 2H), 4.31 (s, 3H), 5.43-5.61 (m, 1H), 6.73 (d, J=7.4 Hz, 1H), 6.92 (t, J=7.8 Hz, 1H), 7.50-7.62 (m, 2H), 8.01 (m, J=13.2 Hz, 3H), 8.59 (d, J=5.2 Hz, 1H), 8.79 (s, 1H), 10.13 (s, 1H).

### Example 455—Synthesis of 1-(4-(1,1-Difluoroethyl)Pyridin-2-yl)-N-(1-(Methyl-D3)-1H-Indazol-7-yl-3-D)-1H-Pyrazole-4-Sulfonamide (I-380)

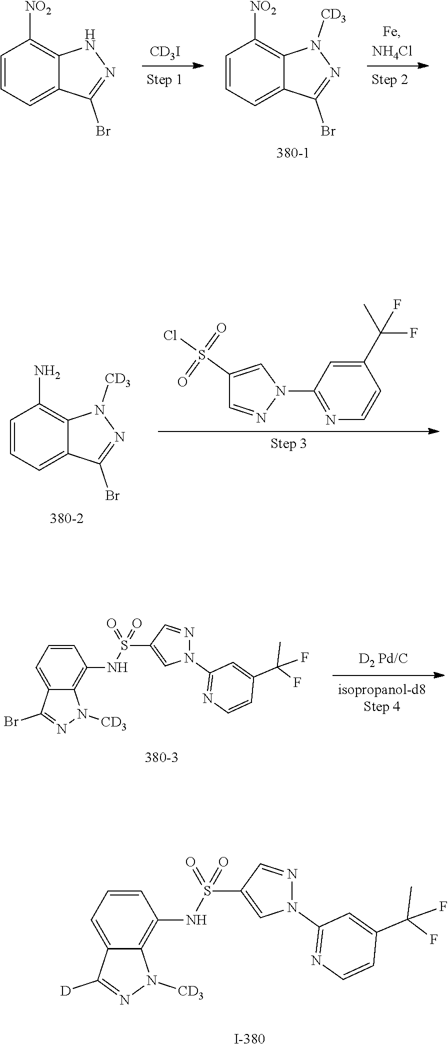

Step 1: Synthesis of 380-1: To a stirred solution of 3-bromo-7-nitro-1H-indazole (1.00 g, 4.132 mmol, 1 equiv) in acetone (10 mL) was added Cs₂CO₃ (2.69 g, 8.264 mmol, 2 equiv) at 0° C. under a nitrogen atmosphere. The resulting mixture was stirred for 30 min at room temperature under a nitrogen atmosphere. To the above mixture was added CD₃I (1.80 g, 12.396 mmol, 3 equiv) dropwise at room temperature. The resulting mixture was stirred for an additional 30 min at room temperature under a nitrogen atmosphere. The reaction was quenched by the addition of sat. NH₄Cl (aq.) (10 mL) at 0° C. The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 60% gradient in 10 min; detector: UV 254 nm. This resulted in 3-bromo-1-(methyl-d₃)-7-nitro-1H-indazole (380-1, 800.00 mg, 67.26%) as a light yellow solid.

Step 2: Synthesis of 380-2: A mixture of 3-bromo-1-(methyl-d₃)-7-nitro-1H-indazole (400.00 mg, 1.544 mmol, 1 equiv), Fe (431.11 mg, 7.720 mmol, 5 equiv) and NH₄Cl (412.93 mg, 7.720 mmol, 5 equiv) in EtOH (40 mL) was stirred for 16 h at 80° C. under a nitrogen atmosphere. The resulting mixture was extracted with EtOAc (3×60 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EA (1:1) to afford 3-bromo-1-(methyl-d₃)-1H-indazol-7-amine (380-2, 250.00 mg, 68.63%) as an off-white solid.

Step 3: Synthesis of 380-3: A mixture of 3-bromo-1-(methyl-d₃)-1H-indazol-7-amine (300.00 mg, 1.310 mmol, 1 equiv) and 1-(4-(1,1-difluoroethyl)pyridin-2-yl)-1H-pyrazole-4-sulfonyl chloride (805.87 mg, 2.620 mmol, 2 equiv) in pyridine (5 mL) was stirred for 30 min at room temperature. The resulting mixture was diluted with water (10 mL) and extracted with EtOAc (3×20 mL). The combined organic layers were washed with brine (1×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 40% to 50% gradient in 10 min; detector: UV 254 nm, to afford N-(3-bromo-1-(methyl-d4)-1H-indazol-7-yl)-1-(4-(1,1-difluoroethyl)pyridin-2-yl)-1H-pyrazole-4-sulfonamide (380-3, 300.00 mg, 44.46%) as an off-white solid.

Step 4: Synthesis of 1-(4-(1,1-difluoroethyl)pyridin-2-yl)-N-(1-(methyl-d3)-1H-indazol-7-yl-3-d)-1H-pyrazole-4-sulfonamide (I-380): To a solution of N-(3-bromo-1-(methyl-d₃)-1H-indazol-7-yl)-1-(4-(1,1-difluoroethyl)pyridin-2-yl)-1H-pyrazole-4-sulfonamide (300.00 mg, 0.600 mmol, 1 equiv) in isopropanol-d₈ (5 mL) was added Pd/C (10%, 6.38 mg) under a nitrogen atmosphere in a 50 mL round-bottom flask. The mixture was hydrogenated at room temperature for 1 h under a hydrogen atmosphere using a hydrogen balloon, filtered through a celite pad and concentrated under reduced pressure. The residue was purified by reversed-phase flash chromatography with the following conditions: column: C18 silica gel; mobile phase: MeCN in water, 50% to 60% gradient in 10 min; detector: UV 254 nm, to afford 1-(4-(1,1-difluoroethyl)pyridin-2-yl)-N-(1-(methyl-d₃)-1H-indazol-7-yl-3-d)-1H-pyrazole-4-sulfonamide (I-380, 124.3 mg, 48.78%) as an off-white solid. LC-MS (ES, m/z): [M+H]⁺=423; ¹H NMR (400 MHz, DMSO-d₆): δ 2.06 (t, J=20.0 Hz, 3H), 6.72 (d, J=8.0 Hz, 1H), 6.97 (t, J=8.0 Hz, 1H), 7.75 (d, J=4.4 Hz, 1H), 7.62 (d, J=8.0 Hz, 1H), 8.07 (s, 1H), 8.09 (s, 1H), 8.69 (d, J=4.0 Hz, 1H), 8.84 (s, 1H), 10.05 (s, 1H).

### Example 456—Synthesis of N-(1-Methyl-1H-Indazol-7-yl)-1-(4-(1,3,3-Trifluorocyclobutyl)Pyridin-2-yl)-1H-Pyrazole-4-Sulfonamide (I-383)

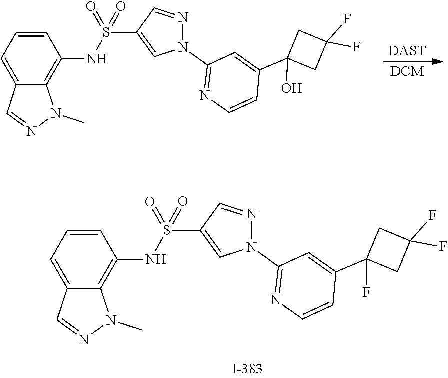

To a solution of 1-[4-(3,3-difluoro-1-hydroxycyclobutyl)pyridin-2-yl]-N-(1-methylindazol-7-yl)pyrazole-4-sulfonamide (90 mg, 0.195 mmol, 1 equiv) in DCM (2 mL) was added DAST (94.52 mg, 0.585 mmol, 3 equiv) at 0° C. The reaction mixture was stirred overnight at room temperature. The reaction was quenched with water/ice (20 mL) at 0° C. The resulting mixture was extracted with CH₂Cl₂ (3×30 mL). The combined organic layers were washed with brine (1×50 mL) and dried over anhydrous Na₂SO₄. After filtration, the filtrate was concentrated under reduced pressure. The crude product (30 mg) was purified by prep-HPLC with the following conditions (column: Xselect CSH C18 OBD column 30*150 mm 5 μm; mobile phase A: water (0.1% FA), mobile phase B: ACN; flow rate: 60 mL/min; gradient: 45% B to 55% B in 7 min; wavelength: 254 nm/220 nm; RT1(min): 6.5) to afford N-(1-methylindazol-7-yl)-1-[4-(1,3,3-trifluorocyclobutyl)pyridin-2-yl]pyrazole-4-sulfonamide (I-383, 6 mg, 6.52%) as an off-white solid. LC-MS (ES, m/z): [M+H]=463; ¹H NMR (400 MHz, CD₃CN): δ 3.27-3.46 (m, 4H), 4.35 (s, 3H), 6.79 (dd, J=1.2, 7.2 Hz, 1H), 6.97 (t, J=7.6 Hz, 1H), 7.50 (d, J=5.6 Hz, 1H), 7.74 (d, J=8.0 Hz, 1H), 7.91 (s, 1H), 8.03 (s, 1H), 8.08 (s, 1H), 8.54 (d, J=5.2 Hz, 1H), 8.79 (s, 1H).

### Example 457—Synthesis of 1-(4-(5-Azaspiro[2.3]Hexan-5-yl)Pyridin-2-yl)-N-(6-Methoxy-1-Methyl-1H-Pyrazolo[4,3-C]Pyridin-7-yl)-1H-Pyrazole-4-Sulfonamide (I-386)

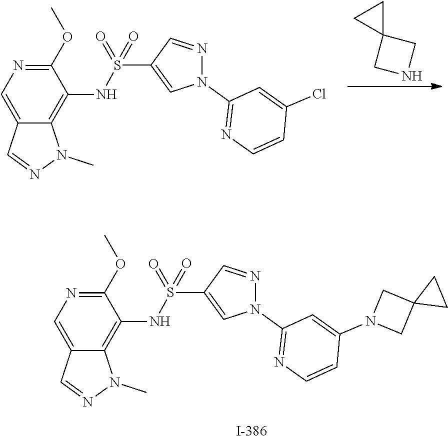 [SMILES: COc1ncc2cnn(C)c2c1NS(=O)(=O)c1cnn(-c2cc(Cl)ccn2)c1 ; C1CC12CNC2 ; COc1ncc2cnn(C)c2c1NS(=O)(=O)c1cnn(-c2cc(N3CC4(CC4)C3)ccn2)c1]

A solution of 1-(4-chloropyridin-2-yl)-N-{6-methoxy-1-methylpyrazolo[4,3-c]pyridin-7-yl}pyrazole-4-sulfonamide (90 mg, 0.214 mmol, 1 equiv), 5-azaspiro[2.3]hexane (35.64 mg, 0.428 mmol, 2 equiv) and Cs₂CO₃ (209.53 mg, 0.642 mmol, 3 equiv) in DMF (2 mL) was stirred overnight at 140° C. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (130 mg) was purified by prep-HPLC with the following conditions (column: XBridge Prep OBD C18 column: 30*150 mm, 5 μm; mobile phase A: water(10 mmol/L NH₄HCO₃+0.05% NH₃·H₂O), mobile phase B: ACN; flow rate: 60 mL/min; gradient: 25% B to 50% B in 7 min; wavelength: 254 nm/220 nm; RT1(min): 8.97) to afford 1-(4-(5-azaspiro[2.3]hexan-5-yl)pyridin-2-yl)-N-(6-methoxy-1-methyl-1H-pyrazolo[4,3-c]pyridin-7-yl)-1H-pyrazole-4-sulfonamide (I-386, 32.6 mg, 32.21%) as a light yellow solid. LC-MS (ES, m/z): [M+H]⁺=467; ¹H NMR (400 MHz, DMSO-d₆): δ 0.71 (s, 4H), 3.43 (s, 3H), 4.10 (s, 4H), 4.21 (s, 3H), 6.40 (dd, J=5.8, 2.2 Hz, 1H), 6.84 (d, J=2.0 Hz, 1H), 7.91 (d, J=0.8 Hz, 1H), 8.05 (d, J=5.8 Hz, 1H), 8.21 (s, 1H), 8.64 (s, 1H), 8.66 (d, J=1.2 Hz, 1H), 9.63 (s, 1H).

### Example 458—Synthesis of N-(6-Methoxy-1-Methyl-1H-Pyrazolo[4,3-C]Pyridin-7-yl)-1-(4-(Piperidin-1-yl)Pyridin-2-yl)-1H-Pyrazole-4-Sulfonamide (I-387)

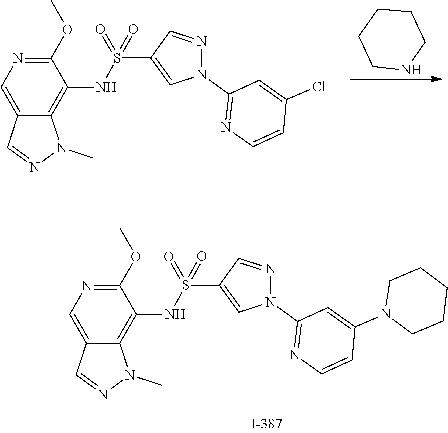 [SMILES: COc1ncc2cnn(C)c2c1NS(=O)(=O)c1cnn(-c2cc(Cl)ccn2)c1 ; C1CCNCC1 ; COc1ncc2cnn(C)c2c1NS(=O)(=O)c1cnn(-c2cc(N3CCCCC3)ccn2)c1]

A solution of 1-(4-chloropyridin-2-yl)-N-{6-methoxy-1-methylpyrazolo[4,3-c]pyridin-7-yl}pyrazole-4-sulfonamide (90 mg, 0.214 mmol, 1 equiv), piperidine (36.51 mg, 0.428 mmol, 2 equiv) and Cs₂CO₃ (209.53 mg, 0.642 mmol, 3 equiv) in DMF (2 mL) was stirred overnight at 140° C. The resulting mixture was diluted with water (20 mL). The resulting mixture was extracted with CH₂Cl₂ (3×20 mL). The combined organic layers were washed with brine (50 mL) and dried over anhydrous Na₂SO₄. After filtration, the filtrate was concentrated under reduced pressure. The crude product (130 mg) was purified by prep-HPLC with the following conditions (column: XBridge Shield RP18 OBD column: 30*150 mm, 5 μm; mobile phase A: water (10 mmol/L NH₄HCO₃+0.05% NH₃H₂O), mobile phase B: ACN; flow rate: 60 mL/min; gradient: 32% B to 55% B in 7 min; wavelength: 254 nm/220 nm; RT1(min): 6.7; number of runs: 2) to afford N-(6-methoxy-1-methyl-1H-pyrazolo[4,3-c]pyridin-7-yl)-1-(4-(piperidin-1-yl)pyridin-2-yl)-1H-pyrazole-4-sulfonamide (I-387, 31.3 mg, 31.10%) as a white solid. LC-MS (ES, m/z): [M+H]⁺=469; ¹H NMR (400 MHz, DMSO-d₆): δ 1.60 (dd, J=9.8 Hz, 16.4, 6H), 3.43 (s, 3H), 3.45 (d, J=7.8 Hz, 4H), 4.21 (s, 3H), 6.87 (dd, J=2.4, 6.0 Hz, 1H), 7.27 (d, J=2.4 Hz, 1H), 7.92 (s, 1H), 8.05 (d, J=6.0 Hz, 1H), 8.22 (s, 1H), 8.65 (s, 2H), 9.84 (s, 1H).

### Example 459—Synthesis of N-(1-Methyl-1H-Indazol-7-yl)-1-(4-(4-Methylpiperidin-1-yl)Pyridin-2-yl)-1H-Pyrazole-4-Sulfonamide (I-390)

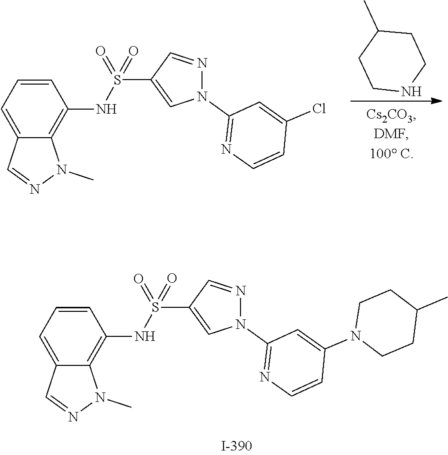

A solution of 1-(4-chloropyridin-2-yl)-N-(1-methylindazol-7-yl)pyrazole-4-sulfonamide (100 mg, 0.257 mmol, 1 equiv), 4-methylpiperidine (127.53 mg, 1.285 mmol, 5 equiv) and Cs₂CO₃ (251.38 mg, 0.771 mmol, 3 equiv) in DMF (5 mL) was stirred overnight at 100° C. under a nitrogen atmosphere. The reaction was quenched with water at room temperature. The resulting mixture was extracted with EtOAc (2×20 mL). The combined organic layers were washed with brine (2×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in N-(1-methylindazol-7-yl)-1-[4-(4-methylpiperidin-1-yl)pyridin-2-yl]pyrazole-4-sulfonamide (I-390, 4.6 mg, 3.96%) as an off-white solid. LC-MS (ES, m/z): [M+H]⁺=452; ¹H NMR (400 MHz, CD₃OD): δ 0.99 (d, J=6.4 Hz, 3H), 1.22-1.31 (m, 2H), 1.66-1.83 (m, 3H), 2.98 (td, J=12.8, 2.6 Hz, 2H), 4.04 (d, J=13.2 Hz, 2H), 4.37 (s, 3H), 6.73 (dd, J=7.4, 1.0 Hz, 1H), 6.81 (dd, J=6.2, 2.6 Hz, 1H), 6.93-7.02 (m, 1H), 7.37 (d, J=2.4 Hz, 1H), 7.70 (dd, J=8.0, 1.0 Hz, 1H), 7.79 (s, 1H), 7.96-8.04 (m, 1H), 8.01 (s, 1H), 8.67 (s, 1H).

### Example 460—Synthesis of 1-(4-(1-Fluoro-3-Methyl-Cyclobutyl)Pyridin-2-yl)-N-(1-Methyl-1H-Indazol-7-yl)-1H-Pyrazole-4-Sulfonamide (I-394)

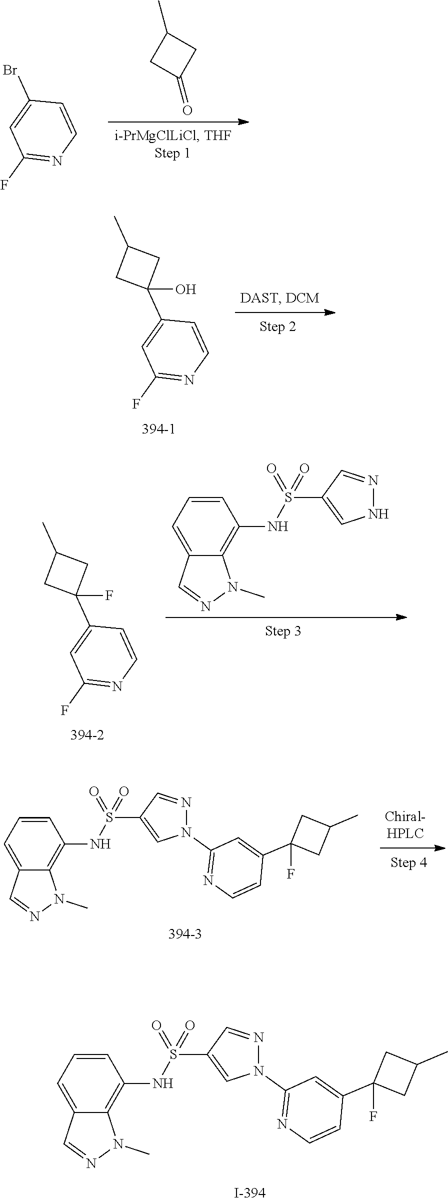

Step 1: Synthesis of 394-1: A solution of 4-bromo-2-fluoropyridine (1.519 g, 8.614 mmol, 1 equiv) in THF (100 mL) was treated with isopropylmagnesium chloride lithium chloride (1328.84 mg, 12.92 mmol, 1.5 equiv) for 30 min at −78° C. under a nitrogen atmosphere followed by the addition of 3-methylcyclobutan-1-one (869.54 mg, 10.34 mmol, 1.20 equiv) dropwise at −60° C. Then the mixture was stirred from −60° C. to room temperature overnight. After the reaction was completed, the reaction was quenched with sat. NH₄Cl (aq.) at room temperature, the aqueous layer was extracted with EtOAc (3×200 mL), washed with brine (1×100 mL), dried over anhydrous Na₂SO₄ and concentrated under reduced pressure to give the crude. The crude was purified by prep-TLC (PE/EA 4:1) to afford 394-1 (1.18 g, 75.59%) as a yellow oil.

Step 2: Synthesis of 394-2: A solution of 394-1 (710 mg, 3.918 mmol, 1 equiv) and DAST (3157.80 mg, 19.590 mmol, 5 equiv) in DCM (40 mL) was stirred overnight from 0° C. to room temperature. After the reaction was completed, the reaction was quenched by the addition of sat. NH₄Cl (aq.) (50 mL) at 0° C. The resulting mixture was extracted with EtOAc (3×50 mL). The combined organic layers were washed with brine (50 mL) and dried over anhydrous Na₂SO₄. After filtration, the filtrate was concentrated under reduced pressure to give the crude. The residue was purified by Prep-TLC (PE/EA 3.5:1) to afford 394-2 (200 mg, 27.86%) as a light yellow oil.

Step 3: Synthesis of 394-3: A solution of 394-2 (92 mg, 0.502 mmol, 1 equiv), N-(1-methylindazol-7-yl)-1H-pyrazole-4-sulfonamide (167.10 mg, 0.602 mmol, 1.2 equiv) and Cs₂CO₃ (409.05 mg, 1.255 mmol, 2.5 equiv) in DMF (5 mL) was stirred overnight at 80° C. After the reaction was complete, the resulting mixture was diluted with water (80 mL). The resulting mixture was extracted with EtOAc (3×100 mL). The combined organic layers were washed with brine (200 mL) and dried over anhydrous Na₂SO₄. After filtration, the filtrate was concentrated under reduced pressure. The residue was purified by reversed-phase flash chromatography with the following conditions: column: C₁₈ silica gel; mobile phase: ACN in water (10 mmol/L NH₄HCO₃), 0% to 100% gradient in 25 min; detector: UV 254 nm to afford 394-3 (200 mg, 90.41%) as a yellow solid.

Step 4: Synthesis of 1-(4-((1-fluoro-3-methylcyclobutyl)pyridin-2-yl)-N-(1-methyl-1H-indazol-7-yl)-1H-pyrazole-4-sulfonamide (I-394): A solution of 394-3 (145 mg, 0.329 mmol, 1 equiv) was purified by chiral-HPLC with the following conditions (column: CHIRALPAKIA3; mobile phase A: hex (0.2% FA): IPA=70:30; flow rate: 1 mL/min; gradient: isocratic; injection volume: 3 mL) to afford 1-(4-((1-fluoro-3-methylcyclobutyl)pyridin-2-yl)-N-(1-methyl-1H-indazol-7-yl)-1H-pyrazole-4-sulfonamide as a single steroeoisomer (I-394, 28.1 mg, 19.38%) as an off-white solid. LC-MS (ES, m/z): [M+1]⁺=441; ¹H NMR (400 MHz, DMSO-d₆): δ 1.18 (d, J=5.2 Hz, 3H), 2.08-2.32 (m, 2H), 2.65-2.78 (m, 3H), 4.30 (d, J=2.4 Hz, 3H), 6.71-6.74 (m, 1H), 6.95 (t, J=7.2 Hz, 1H), 7.54 (d, J=4.0 Hz, 1H), 7.64 (d, J=8.0 Hz, 1H), 7.94 (s, 1H), 8.03-8.14 (m, 2H), 8.55 (d, J=2.8 Hz, 1H), 8.80 (s, 1H), 10.19 (s, 1H).

### Example 461—Synthesis of 1-(4-(1-Fluoro-3-Methyl-Cyclobutyl)Pyridin-2-yl)-N-(1-Methyl-1H-Indazol-7-yl)-1H-Pyrazole-4-Sulfonamide (I-395)

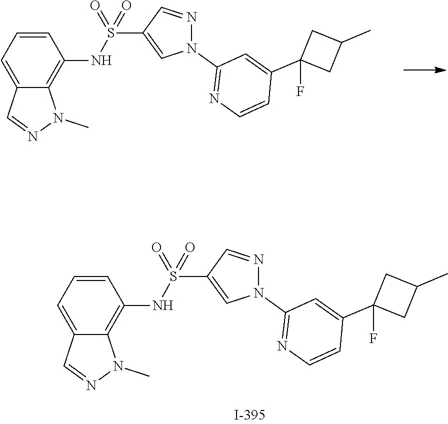 [SMILES: CC1CC(F)(c2ccnc(-n3cc(S(=O)(=O)Nc4cccc5cnn(C)c45)cn3)c2)C1 ; CC1CC(F)(c2ccnc(-n3cc(S(=O)(=O)Nc4cccc5cnn(C)c45)cn3)c2)C1]

A solution of 1-[4-(1-fluoro-3-methylcyclobutyl)pyridin-2-yl]-N-(1-methylindazol-7-yl)pyrazole-4-sulfonamide (145 mg, 0.329 mmol, 1 equiv) was purified by chiral-HPLC with the following conditions (column: CHIRALPAKIA3; mobile phase A: hex (0.2% FA): IPA=70:30; flow rate: 1 mL/min; gradient: isocratic; injection volume: 3 mL) to afford 1-(4-(1-fluoro-3-methylcyclobutyl)pyridin-2-yl)-N-(1-methyl-1H-indazol-7-yl)-1H-pyrazole-4-sulfonamide as a single steroeoisomer (1-395, 28.1 mg, 19.38%) as an off-white solid. LC-MS (ES, m/z): [M+1]⁺=441; ¹H NMR (400 MHz, DMSO-d₆): δ 1.24 (d, J=6.0 Hz, 3H), 2.25-2.33 (m, 3H), 2.75-2.79 (m, 2H), 4.29 (s, 3H), 6.71 (t, J=6.4 Hz, 1H), 6.97 (t, J=8.0 Hz, 1H), 7.57-7.59 (m, 1H), 7.70 (d, J=8.0 Hz, 1H), 7.98 (s, 1H), 8.04 (s, 1H), 8.09 (s, 1H), 8.57 (d, J=5.2 Hz, 1H), 8.82 (s, 1H), 10.04 (s, 1H).

### Example 462—Synthesis of 1-(4-(3-Methoxycyclobutyl)Pyridin-2-yl)-N-(1-Methyl-1H-Indazol-7-yl)-1H-Pyrazole-4-Sulfonamide (I-399)

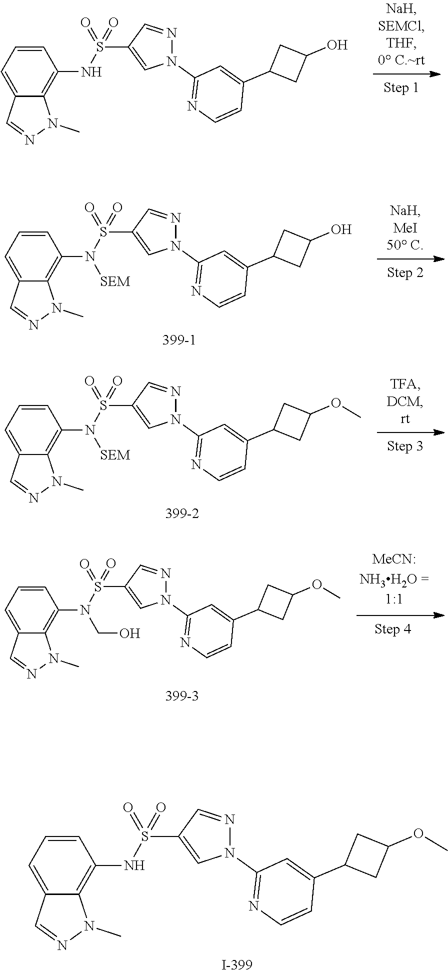 [SMILES: Cn1ncc2cccc(NS(=O)(=O)c3cnn(-c4cc(C5CC(O)C5)ccn4)c3)c21 ; Cn1ncc2cccc(N(COCC[Si](C)(C)C)S(=O)(=O)c3cnn(-c4cc(C5CC(O)C5)ccn4)c3)c21 ; COC1CC(c2ccnc(-n3cc(S(=O)(=O)N(COCC[Si](C)(C)C)c4cccc5cnn(C)c45)cn3)c2)C1 ; COC1CC(c2ccnc(-n3cc(S(=O)(=O)N(CO)c4cccc5cnn(C)c45)cn3)c2)C1 ; COC1CC(c2ccnc(-n3cc(S(=O)(=O)Nc4cccc5cnn(C)c45)cn3)c2)C1]

Step 1: Synthesis of 399-1: A solution of 1-[4-(3-hydroxycyclobutyl)pyridin-2-yl]-N-(1-methylindazol-7-yl)pyrazole-4-sulfonamide (55 mg, 0.130 mmol, 1 equiv) in THE (3 mL) was treated with NaH (3.73 mg, 0.156 mmol, 1.2 equiv) at 0° C. followed by the addition of SEM-C1 (21.60 mg, 0.130 mmol, 1 equiv) in portions at 0° C. The resulting mixture was stirred for 2 h at room temperature under a nitrogen atmosphere. The reaction was quenched with ice at 0° C. The resulting mixture was extracted with EtOAc (2×50 mL). The combined organic layers were washed with brine (1×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. This resulted in 1-[4-(3-hydroxycyclobutyl)pyridin-2-yl]-N-(1-methylindazol-7-yl)-N-{[2 (trimethylsilyl)ethoxy]methyl}pyrazole-4-sulfonamide (399-1, 35 mg, 48.69%) as a light yellow oil.

Step 2: Synthesis of 399-2: A solution of 1-[4-(3-hydroxycyclobutyl)pyridin-2-yl]-N-(1-methylindazol-7-yl)-N-{[2-(trimethylsilyl)ethoxy]methyl}pyrazole-4-sulfonamide (25 mg, 0.045 mmol, 1 equiv) in THE (2 mL) was treated with NaH (2.16 mg, 0.090 mmol, 2 equiv) for 0.5 h at 0° C. under a nitrogen atmosphere followed by the addition of Mel (19.19 mg, 0.135 mmol, 3 equiv) in portions at 0° C. The resulting mixture was stirred overnight at 50° C. under a nitrogen atmosphere. The reaction was quenched with ice at 0° C. The resulting mixture was extracted with EtOAc (2×30 mL). The combined organic layers were washed with brine (1×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. This resulted in 1-[4-(3-methoxycyclobutyl)pyridin-2-yl]-N-(1-methylindazol-7-yl)-N-{[2-(trimethylsilyl)ethoxy]methyl}pyrazole-4-sulfonamide (399-2, 12 mg, 46.82%) as a light yellow oil.

Step 3: Synthesis of 399-3: A solution of 1-[4-(3-methoxycyclobutyl)pyridin-2-yl]-N-(1-methylindazol-7-yl)-N-{[2-(trimethylsilyl)ethoxy]methyl}pyrazole-4-sulfonamide (12 mg, 0.021 mmol, 1 equiv) in DCM (3 mL) was added TFA (1 mL). The resulting mixture was stirred for 4 h at room temperature under a nitrogen atmosphere. The resulting mixture was concentrated under reduced pressure. This resulted in N-(hydroxymethyl)-1-[4-(3-methoxycyclobutyl)pyridin-2-yl]-N-(1-methylindazol-7-yl)pyrazole-4-sulfonamide (399-3, 10 mg, 101.16%) as a light yellow oil. The crude product was used in the next step directly without further purification.

Step 4: Synthesis of 1-(4-(3-methoxycyclobutyl)pyridin-2-yl)-N-(1-methyl-1H-indazol-7-yl)-1H-pyrazole-4-sulfonamide (I-399): A solution of N-(hydroxymethyl)-1-[4-(3-methoxycyclobutyl)pyridin-2-yl]-N-(1-methylindazol-7-yl)pyrazole-4-sulfonamide (10 mg, 0.021 mmol, 1 equiv) in NH₃·H₂O (1 mL) and MeCN (1 mL) was stirred for 2 h at room temperature under a nitrogen atmosphere. The resulting mixture was concentrated under reduced pressure. The crude product (8 mg) was purified by prep-HPLC with the following conditions (column: Xcelect CSH F-pheny OBD column: 19*250 mm, 5 μm; mobile phase A: water (0.1% FA), mobile phase B: ACN; flow rate: 25 mL/min; gradient: 32% B to 48% B in 9 min; wavelength: 254 nm/220 nm; RT1(min): 9; number of runs: 4) to afford 1-[4-(3-methoxycyclobutyl)pyridin-2-yl]-N-(1-methylindazol-7-yl)pyrazole-4-sulfonamide (I-399, 2.1 mg, 21.81%) as a white solid. LC-MS (ES, m/z): [M+H]⁺=439; ¹H NMR (400 MHz, DMSO-d₆): δ 1.95 (m, J=10.4, 5.4, 2.6 Hz, 2H), 2.72 (m, J=11.6, 8.6, 4.0 Hz, 2H), 3.18 (s, 3H), 3.13-3.25 (m, 1H), 4.28 (s, 3H), 3.84-3.96 (m, 1H), 6.70 (d, J=7.4 Hz, 1H), 6.97 (t, J=7.8 Hz, 1H), 7.36 (m, J=5.2, 1.6 Hz, 1H), 7.70 (d, J=8.0 Hz, 1H), 7.82 (s, 1H), 8.01 (s, 1H), 8.09 (s, 1H), 8.42 (d, J=5.0 Hz, 1H), 8.79 (s, 1H), 10.01 (s, 1H).

### Example 463—Synthesis of (S)—N-(1-Methyl-1H-Indazol-7-yl)-1-(4-(3-Methylpyrrolidin-1-yl)Pyridin-2-yl)-1H-Pyrazole-4-Sulfonamide (I-400)

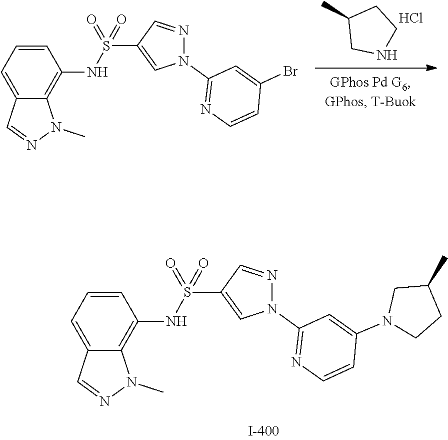

To a stirred solution of 1-(4-bromopyridin-2-yl)-N-(1-methylindazol-7-yl) pyrazole-4-sulfonamide (100 mg, 0.231 mmol, 1 equiv) and (3S)-3-methylpyrrolidin-1-ium chloride (168.40 mg, 1.386 mmol, 6 equiv) in dioxane (4 mL) were added {2-[2,6-bis(propan-2-yl) phenyl]-6-(tert-butoxy)-3-methoxyphenyl} dicyclohexylphosphane (12.39 mg, 0.023 mmol, 0.1 equiv), GPhos Pd G6 TES (21.20 mg, 0.023 mmol, 0.1 equiv) and t-BuOK (77.70 mg, 0.693 mmol, 3 equiv) in portions at room temperature under a nitrogen atmosphere. The resulting mixture was stirred for 2 h at 100° C. under a nitrogen atmosphere. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in N-(1-methylindazol-7-yl)-1-{4-[(3S)-3-methylpyrrolidin-1-yl] pyridin-2-yl} pyrazole-4-sulfonamide (I-400, 44.4 mg, 43.79%) as an off-white solid. LC-MS (ES, m/z): [M+H]⁺=438; ¹H NMR (400 MHz, DMSO-d₆): δ 1.09 (d, J=6.6 Hz, 3H), 1.56-1.68 (m, 1H), 2.09-2.21 (m, 1H), 2.34-2.43 (m, 1H), 2.92 (dd, J=12.0, 8.0 Hz, 1H), 3.30-3.40 (m, 1H), 3.42-3.49 (m, 1H), 3.53 (d, J=8.0 Hz, 1H), 4.28 (s, 3H), 6.52 (dd, J=6.0, 4.0 Hz, 1H), 6.70 (dd, J=8.0, 1.0 Hz, 1H), 6.93-7.01 (m, 2H), 7.70 (d, J=8.0 Hz, 1H), 7.93 (d, J=0.8 Hz, 1H), 8.00 (d, J=4.0 Hz, 1H), 8.09 (s, 1H), 8.72 (d, J=0.8 Hz, 1H).

### Example 464—Synthesis of (R)—N-(1-Methyl-1H-Indazol-7-yl)-1-(4-(3-Methylpyrrolidin-1-yl)Pyridin-2-yl)-1H-Pyrazole-4-Sulfonamide (I-401)

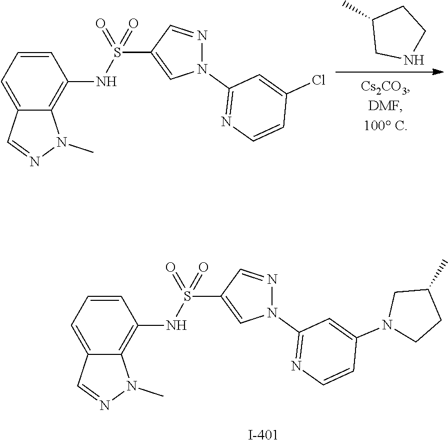

A solution of 1-(4-bromopyridin-2-yl)-N-(1-methylindazol-7-yl)pyrazole-4-sulfonamide (100 mg, 0.231 mmol, 1 equiv), (3R)-3-methylpyrrolidine (98.26 mg, 1.155 mmol, 5 equiv) and Cs₂CO₃ (225.59 mg, 0.693 mmol, 3 equiv) in DMF (5 mL) was stirred overnight at 100° C. under a nitrogen atmosphere. The reaction was quenched with water at room temperature. The resulting mixture was extracted with EtOAc (2×20 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in N-(1-methylindazol-7-yl)-1-{4-[(3R)-3-methylpyrrolidin-1-yl]pyridin-2-yl}pyrazole-4-sulfonamide (I-401, 12.9 mg, 12.77%) as an off-white solid. LC-MS (ES, m/z): [M+H]⁺=438; H NMR (400 MHz, DMSO-d₆): δ 1.09 (d, J=6.6 Hz, 3H), 1.55-1.69 (m, 1H), 2.12 (dd, J=10.8, 6.8 Hz, 1H), 2.39 (q, J=7.4 Hz, 1H), 2.92 (dd, J=10.0, 7.8 Hz, 1H), 3.2-3.59 (m, 3H), 4.28 (s, 3H), 6.51 (dd, J=6.0, 2.2 Hz, 1H), 6.67-6.73 (m, 1H), 6.92-7.00 (m, 2H), 7.65 (s, 1H), 7.92 (d, J=0.8 Hz, 1H), 8.00 (d, J=6.0 Hz, 1H), 8.07 (s, 1H), 8.70 (s, 1H), 9.96 (s, 1H).

### Example 465—Synthesis of 1-(4-(5-Azaspiro[2.4]Heptan-5-yl)Pyridin-2-yl)-N-(1-Methyl-1H-Indazol-7-yl)-1H-Pyrazole-4-Sulfonamide (I-410)

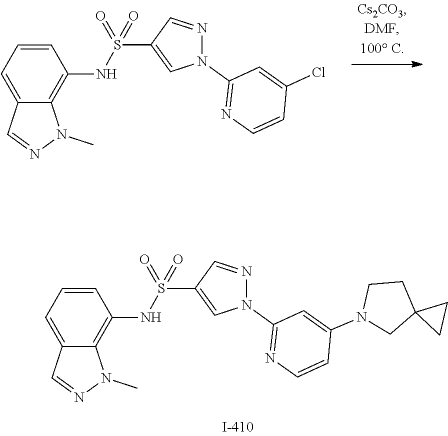 [SMILES: Cn1ncc2cccc(NS(=O)(=O)c3cnn(-c4cc(Cl)ccn4)c3)c21 ; Cn1ncc2cccc(NS(=O)(=O)c3cnn(-c4cc(N5CCC6(CC6)C5)ccn4)c3)c21]

To a stirred solution of 1-(4-chloropyridin-2-yl)-N-(1-methylindazol-7-yl)pyrazole-4-sulfonamide (100 mg, 0.257 mmol, 1 equiv) and 5-azaspiro[2.4]heptane (124.94 mg, 1.285 mmol, 5 equiv) in DMF (2 mL) was added Cs₂CO₃ (335.18 mg, 1.028 mmol, 4 equiv). The resulting mixture was stirred overnight at 100° C. under a nitrogen atmosphere. The reaction was quenched with water at room temperature. The resulting mixture was extracted with EtOAc (2×50 mL). The combined organic layers were washed with brine (40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in 1-(4-{5-azaspiro[2.4]heptan-5-yl}pyridin-2-yl)-N-(1-methylindazol-7-yl)pyrazole-4-sulfonamide (I-410, 32.3 mg, 27.69%) as an off-white solid. LC-MS (ES, m/z): [M+H]⁺=450; ¹H NMR (400 MHz, CDCl₃): δ 0.70 (s, 4H), 1.96-2.04 (m, 2H), 3.34 (s, 2H), 3.61 (t, J=6.8 Hz, 2H), 4.42 (s, 3H), 6.37 (d, J=5.8 Hz, 1H), 6.69 (m, 1H), 6.80 (d, J=7.2 Hz, 1H), 6.95 (t, J=7.6 Hz, 1H), 7.02 (s, 1H), 7.64-7.73 (m, 2H), 7.97-8.05 (m, 2H), 9.06 (s, 1H).

### Example 466—Synthesis of 1-(4-(3,3-Dimethylpyrrolidin-1-yl)Pyridin-2-yl)-N-(1-Methyl-1H-Indazol-7-yl)-1H-Pyrazole-4-Sulfonamide (I-421)

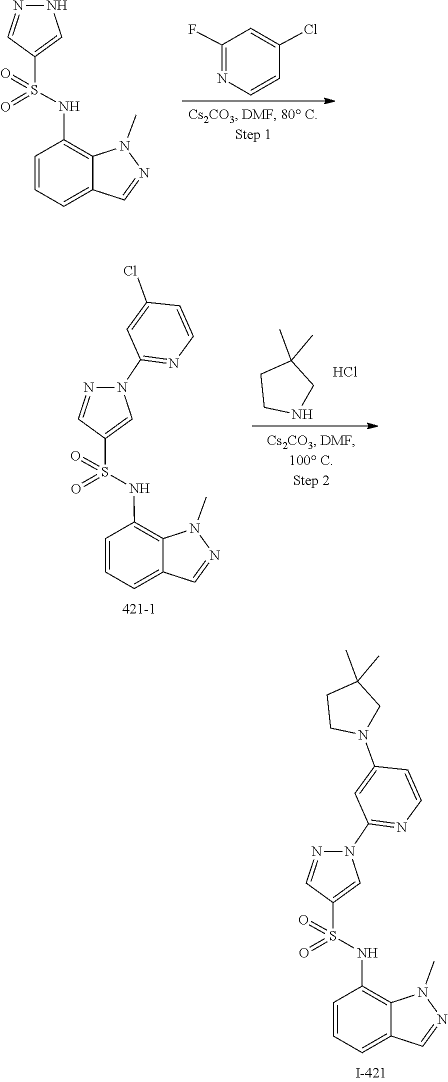

Step 1: Synthesis of 421-1: To a stirred solution of 4-chloro-2-fluoropyridine (0.5 g, 4.327 mmol, 1.2 equiv) and N-(1-methylindazol-7-yl)-1H-pyrazole-4-sulfonamide (1 g, 3.606 mmol, 1.00 equiv) in DMF (10 mL) was added Cs₂CO₃ (3.52 g, 10.818 mmol, 3 equiv) in portions at room temperature under a nitrogen atmosphere. The resulting mixture was stirred for 2 h at 80° C. under a nitrogen atmosphere. The resulting mixture was diluted with water (50 mL). The resulting mixture was extracted with EtOAc (3×100 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 35% to 40% gradient in 10 min; detector: UV 254 nm. The resulting mixture was concentrated under reduced pressure. This resulted in 1-(4-chloropyridin-2-yl)-N-(1-methylindazol-7-yl) pyrazole-4-sulfonamide (421-1, 1100 mg, 78.45%) as a pink solid.

Step 2: Synthesis of 1-(4-(3,3-dimethylpyrrolidin-1-yl)pyridin-2-yl)-N-(1-methyl-1H-indazol-7-yl)-1H-pyrazole-4-sulfonamide (I-421): To a stirred solution of 1-(4-chloropyridin-2-yl)-N-(1-methylindazol-7-yl) pyrazole-4-sulfonamide (80 mg, 0.206 mmol, 1 equiv) and 3,3-dimethylpyrrolidine hydrochloride (139.54 mg, 1.030 mmol, 5 equiv) in DMF (3 mL) was added Cs₂CO₃ (201.11 mg, 0.618 mmol, 3 equiv) in portions at room temperature under a nitrogen atmosphere. The resulting mixture was stirred for 3 h at 100° C. under a nitrogen atmosphere. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 50% gradient in 10 min; detector: UV 254 nm. The resulting mixture was concentrated under reduced pressure. This resulted in 1-[4-(3,3-dimethylpyrrolidin-1-yl) pyridin-2-yl]-N-(1-methylindazol-7-yl) pyrazole-4-sulfonamide (I-421, 33.8 mg, 35.25%) as an off-white solid. LC-MS (ES, m/z): [M+H]⁺=452; ¹H NMR (400 MHz, DMSO-d₆): δ 1.09 (s, 6H), 1.77 (t, J=8.0 Hz, 2H), 3.10 (s, 2H), 3.40 (t, J=7.0 Hz, 2H), 4.41 (s, 3H), 6.40 (dd, J=4.0, 2.0 Hz, 1H), 6.66 (t, J=8.0 Hz, 1H), 6.76 (dd, J=8.0, 1.0 Hz, 1H), 6.84-6.88 (m, 2H), 7.67 (s, 1H), 7.75 (s, 1H), 7.94 (d, J=8.0 Hz, 1H), 8.45 (s, 1H).

### Example 467—Synthesis of (S)-1-(4-(3-Fluoropyrrolidin-1-yl)Pyridin-2-yl)-N-(1-Methyl-1H-Indazol-7-yl)-1H-Pyrazole-4-Sulfonamide (I-429)

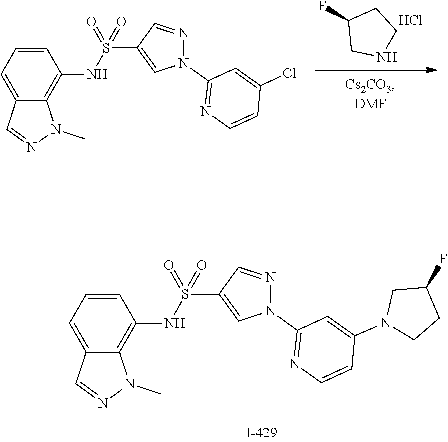 [SMILES: Cn1ncc2cccc(NS(=O)(=O)c3cnn(-c4cc(Cl)ccn4)c3)c21 ; F[C@H]1CCNC1 ; Cl ; Cn1ncc2cccc(NS(=O)(=O)c3cnn(-c4cc(N5CC[C@H](F)C5)ccn4)c3)c21]

To a stirred solution of 1-(4-chloropyridin-2-yl)-N-(1-methylindazol-7-yl) pyrazole-4-sulfonamide (120 mg, 0.309 mmol, 1 equiv) and (3S)-3-fluoropyrrolidine hydrochloride (193.77 mg, 1.545 mmol, 5 equiv) in DMF (4 mL) was added Cs₂CO₃ (301.66 mg, 0.927 mmol, 3 equiv) in portions at room temperature under a nitrogen atmosphere. The resulting mixture was stirred for 8 h at 100° C. under a nitrogen atmosphere. The resulting mixture was diluted with water (15 mL). The resulting mixture was extracted with EtOAc (3×15 mL). The combined organic layers were washed with brine (1×10 mL) and dried over anhydrous Na₂SO₄. The residue was purified by reversed-phase flash chromatography with the following conditions: column: C18 silica gel; mobile phase: MeCN in water (0.1% FA), 40% to 50% gradient in 10 min; detector: UV 254 nm. The resulting mixture was concentrated under reduced pressure. This resulted in 1-{4-[(3S)-3-fluoropyrrolidin-1-yl] pyridin-2-yl}-N-(1-methylindazol-7-yl) pyrazole-4-sulfonamide (I-429, 12.8 mg, 9.08%) as an off-white solid. LC-MS (ES, m/z): [M+H]⁺=442; ¹H NMR (400 MHz, DMSO-d₆): δ 2.29 (dd, J=14.2, 6.0 Hz, 2H), 3.42-3.50 (m, 1H), 3.57-3.63 (m, 2H), 3.69 (m, 1H), 4.28 (s, 3H), 5.43 (m, 1H), 6.60 (dd, J=8.0, 2.0 Hz, 1H), 6.70 (dd, J=7.4, 1.0 Hz, 1H), 6.95-7.03 (m, 2H), 7.69 (d, J=8.0 Hz, 1H), 7.95 (d, J=0.8 Hz, 1H), 8.03-8.09 (m, 2H), 8.73 (s, 1H), 9.97 (s, 1H).

### Example 468—Synthesis of (R)-1-(4-(3-Fluoropyrrolidin-1-yl)Pyridin-2-yl)-N-(1-Methyl-1H-Indazol-7-yl)-1H-Pyrazole-4-Sulfonamide (I-430)

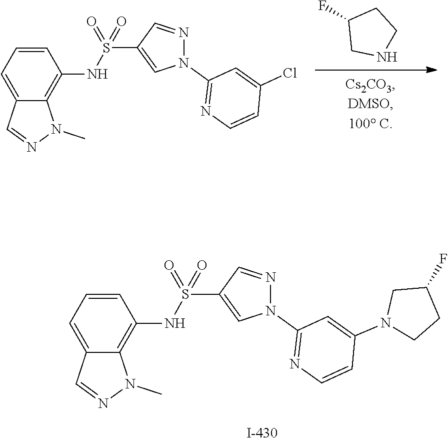 [SMILES: Cn1ncc2cccc(NS(=O)(=O)c3cnn(-c4cc(Cl)ccn4)c3)c21 ; F[C@@H]1CCNC1 ; Cn1ncc2cccc(NS(=O)(=O)c3cnn(-c4cc(N5CC[C@@H](F)C5)ccn4)c3)c21]

A mixture of 1-(4-chloropyridin-2-yl)-N-(1-methylindazol-7-yl)pyrazole-4-sulfonamide (100 mg, 0.257 mmol, 1 equiv), (3R)-3-fluoropyrrolidine (114.59 mg, 1.285 mmol, 5 equiv) and Cs₂CO₃ (251.38 mg, 0.771 mmol, 3 equiv) in DMSO (3 mL) was stirred overnight at 100° C. under a nitrogen atmosphere. The reaction was quenched with water at room temperature. The resulting mixture was extracted with EtOAc (2×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 1-{4-[(3R)-3-fluoropyrrolidin-1-yl]pyridin-2-yl}-N-(1-methylindazol-7-yl)pyrazole-4-sulfonamide (I-430, 46.9 mg, 41.31%) as an off-white solid. LC-MS (ES, m/z): [M+H]⁺=442; ¹H NMR (400 MHz, DMSO-d₆): 2.12-2.38 (m, 2H), 3.51-3.40 (m, 1H), 3.64-3.56 (m, 2H), 3.69 (m, 1H), 4.28 (s, 3H), 5.43-5.56 (m, 1H), 6.59 (dd, J=5.8, 2.4 Hz, 1H), 6.69 (d, J=7.4 Hz, 1H), 6.97 (t, J=7.6 Hz, 1H) 7.03 (d, J=2.2 Hz, 1H), 7.67 (d, J=8.0 Hz, 1H), 7.94 (s, 1H), 8.02-8.10 (m, 2H), 8.72 (s, 1H), 9.99 (s, 1H).

### Example 469—Synthesis of 1-(4-(3,3-Difluoropyrrolidin-1-yl)Pyridin-2-yl)-N-(1-Methyl-1H-Indazol-7-yl)-1H-Pyrazole-4-Sulfonamide (I-431)

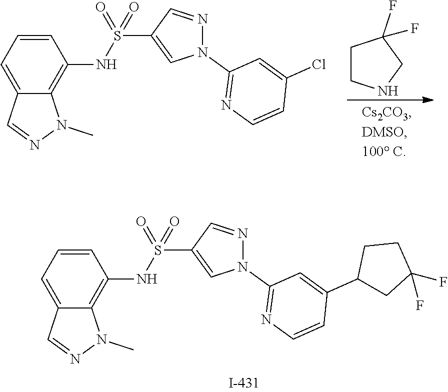

A solution of 1-(4-chloropyridin-2-yl)-N-(1-methylindazol-7-yl)pyrazole-4-sulfonamide (100 mg, 0.257 mmol, 1 equiv), 3,3-difluoropyrrolidine (137.73 mg, 1.285 mmol, 5 equiv) and Cs₂CO₃ (251.38 mg, 0.771 mmol, 3 equiv) in DMSO (2 mL) was stirred overnight at 100° C. under a nitrogen atmosphere. The reaction was quenched with water at room temperature. The resulting mixture was extracted with EtOAc (2×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 1-[4-(3,3-difluoropyrrolidin-1-yl)pyridin-2-yl]-N-(1-methylindazol-7-yl)pyrazole-4-sulfonamide (I-431, 7.1 mg, 6.01%) as an off-white solid. LC-MS (ES, m/z): [M+H]⁺=460; ¹H NMR (400 MHz, DMSO-d₆): δ 2.57 (tt, J=13.6, 7.2 Hz, 2H), 3.69 (t, J=7.4 Hz, 2H), 3.80 (t, J=12.6 Hz, 2H), 4.42 (s, 3H), 6.34-6.42 (m, 2H), 6.78 (d, J=7.2 Hz, 1H), 6.96 (t, J=7.6 Hz, 1H), 7.09 (d, J=2.0 Hz, 1H), 7.66-7.75 (m, 2H), 8.01 (s, 1H), 8.09 (d, J=5.8 Hz, 1H), 8.91 (s, 1H).

### Example 470—Synthesis of 1-(4-(3,3-Difluoropyrrolidin-1-yl)Pyridin-2-yl)-N-(6-Methoxy-1-Methyl-1H-Indazol-7-yl)-1H-Pyrazole-4-Sulfonamide (I-435)

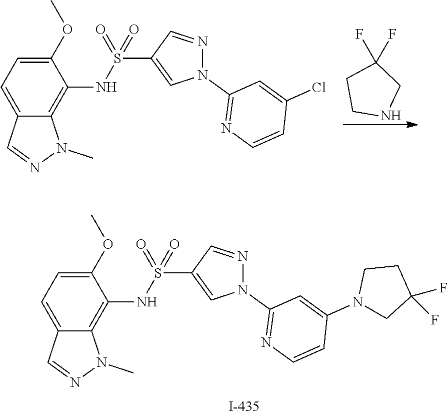

To a stirred mixture of 1-(4-chloropyridin-2-yl)-N-(6-methoxy-1-methylindazol-7-yl)pyrazole-4-sulfonamide (50 mg, 0.119 mmol, 1 equiv) and 3,3-difluoropyrrolidine (63.93 mg, 0.595 mmol, 5 equiv) in DMSO (5 mL) was added Cs₂CO₃ (116.68 mg, 0.357 mmol, 3 equiv) in portions at 140° C. under a nitrogen atmosphere. The resulting mixture was stirred for an additional 3 h at 140° C. The crude product (50 mg) was purified by prep-HPLC with the following conditions (column: XBridge Prep OBD C18 column: 19*250 mm, 5 μm; mobile phase A: water (10 mmol/L NH₄HCO₃+0.05% NH₃H₂O), mobile phase B: MeOH; flow rate: 25 mL/min; gradient: 46% B to 71% B in 10 min; wavelength: 254 nm/220 nm; RT1(min): 9.23; number of runs: 2) to afford 1-(4-(3,3-difluoropyrrolidin-1-yl)pyridin-2-yl)-N-(6-methoxy-1-methyl-1H-indazol-7-yl)-1H-pyrazole-4-sulfonamide (I-435, 2.3 mg, 3.89%) as a white solid. LC-MS (ES, m/z): [M+1]=490; ¹H NMR (400 MHz, chloroform-d): δ 2.50-2.60 (m, 2H), 3.40 (s, 3H), 3.67 (t, J=7.2 Hz, 2H), 3.78 (t, J=12.8 Hz, 2H), 4.40 (s, 3H), 6.36 (dd, J=5.8, 2.4 Hz, 1H), 6.44 (s, 1H), 6.66 (d, J=8.8 Hz, 1H), 7.03 (d, J=2.4 Hz, 1H), 7.60 (d, J=8.8 Hz, 1H), 7.65 (s, 1H), 7.93 (s, 1H), 8.07 (d, J=5.8 Hz, 1H), 8.73 (s, 1H).

### Example 471—Synthesis of 1-(4-(3-Fluoroazetidin-1-yl)Pyridin-2-yl)-N-(1-Methyl-1H-Indazol-7-yl)-1H-Pyrazole-4-Sulfonamide (I-438)

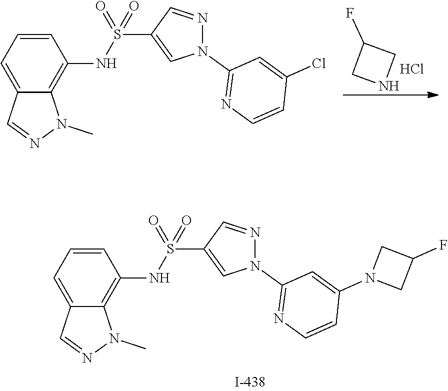

A solution of 1-(4-chloropyridin-2-yl)-N-(1-methylindazol-7-yl)pyrazole-4-sulfonamide (100 mg, 0.257 mmol, 1 equiv), Cs₂CO₃ (251.38 mg, 0.771 mmol, 3 equiv) and 3-fluoroazetidine hydrochloride (57.37 mg, 0.514 mmol, 2 equiv) in DMSO (4 mL) was stirred overnight at 140° C. under a nitrogen atmosphere. The resulting mixture was diluted with water (10 mL) and extracted with EtOAc (3×10 mL). The combined organic layers were washed with brine (1×30 mL) and dried over anhydrous Na₂SO₄. After filtration, the filtrate was concentrated under reduced pressure. The crude product (80 mg) was purified by prep-HPLC with the following conditions (column: XBridge Shield RP18 OBD column: 30*150 mm, 7 m; mobile phase A: water (10 mmol/L NH₄HCO₃+0.05% NH₃H₄O), mobile phase B: ACN; flow rate: 60 mL/min; gradient: 17% B to 40% B in 7 min; wavelength: 254 nm/222 nm; RT1(min): 7; number of runs: 5) to afford 1-[4-(3-fluoroazetidin-1-yl)pyridin-2-yl]-N-(1-methylindazol-7-yl)pyrazole-4-sulfonamide (I-438, 49.3 mg, 44.49%) as a white solid. LC-MS (ES, m/z): [M+1]=428; ¹H NMR (400 MHz, DMSO-d₆): δ 4.10-4.27 (m, 2H), 4.28 (s, 3H), 4.31-4.40 (m, 2H), 5.53 (d, J=57.2 Hz, 1H), 6.47 (dd, J=2.4, 5.6 Hz, 1H), 6.69 (d, J=7.2 Hz, 1H), 6.91 (d, J=2.4 Hz, 1H), 6.97 (t, J=7.6 Hz, 1H), 7.68 (d, J=8.0 Hz, 1H), 7.94 (s, 1H), 8.06 (s, 1H), 8.08 (s, 1H), 8.71 (s, 1H), 9.98 (s, 1H).

### Example 472—Synthesis of 1-(4-(3,3-Difluoroazetidin-1-yl)Pyridin-2-yl)-N-(1-Methyl-1H-Indazol-7-yl)-1H-Pyrazole-4-Sulfonamide (I-439)

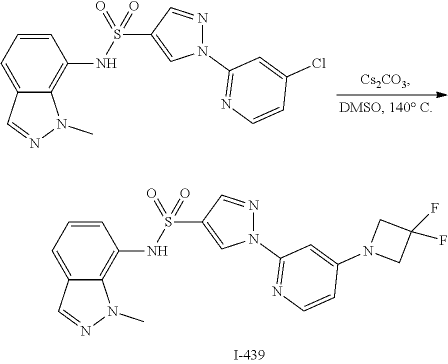 [SMILES: Cn1ncc2cccc(NS(=O)(=O)c3cnn(-c4cc(Cl)ccn4)c3)c21 ; Cn1ncc2cccc(NS(=O)(=O)c3cnn(-c4cc(N5CC(F)(F)C5)ccn4)c3)c21]

To a stirred solution of 1-(4-chloropyridin-2-yl)-N-(1-methylindazol-7-yl)pyrazole-4-sulfonamide (100 mg, 0.257 mmol, 1 equiv) and 3,3-difluoroazetidine hydrochloride (99.94 mg, 0.771 mmol, 3.00 equiv) in DMSO (2 mL) was added Cs₂CO₃ (251.38 mg, 0.771 mmol, 3 equiv). The resulting mixture was stirred overnight at 140° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product (80 mg) was purified by prep-HPLC with the following conditions (column: XBridge Shield RP18 OBD column: 30*150 mm, 5 μm; mobile phase A: water (0.1% FA), mobile phase B: ACN; flow rate: 60 mL/min; gradient: 35% B to 50% B in 8 min; wavelength: 254 nm/220 nm; RT1(min): 7.8) to afford 1-[4-(3,3-difluoroazetidin-1-yl)pyridin-2-yl]-N-(1-methylindazol-7-yl) pyrazole-4-sulfonamide (I-439, 36.3 mg, 31.62%) as an off-white solid. LC-MS (ES, m/z): [M+1]⁺=446; ¹H NMR (400 MHz, DMSO-d₆): δ 4.28 (s, 3H), 4.54 (t, J=12.4 Hz, 4H), 6.57 (dd, J=2.4, 5.6 Hz, 1H), 6.68 (dd, J=1.2, 7.6 Hz, 1H), 6.97 (t, J=7.6 Hz, 1H), 7.03 (d, J=2.0 Hz, 1H), 7.70 (dd, J=1.2, 8.0 Hz, 1H), 7.97 (d, J=0.8 Hz, 1H), 8.09 (s, 1H), 8.14 (d, J=5.6 Hz, 1H), 8.74 (d, J=0.8 Hz, 1H), 10.00 (s, 1H).

### Example 473—Synthesis of 1-(4-(3-Fluoro-3-Methylazetidin-1-yl)Pyridin-2-yl)-N-(1-Methyl-1H-Indazol-7-yl)-1H-Pyrazole-4-Sulfonamide (I-440)

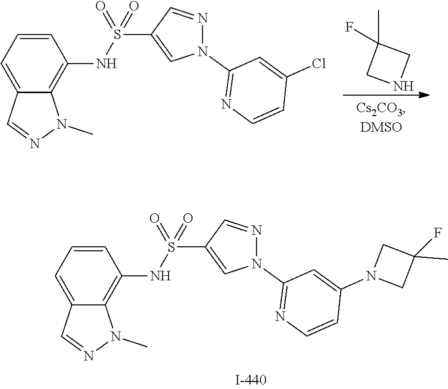

A solution of 1-(4-chloropyridin-2-yl)-N-(1-methylindazol-7-yl)pyrazole-4-sulfonamide (100 mg, 0.257 mmol, 1 equiv), 3-fluoro-3-methylazetidine (45.84 mg, 0.514 mmol, 2 equiv) and Cs₂CO₃ (251.38 mg, 0.771 mmol, 3 equiv) in DMSO (3 mL) was stirred overnight at 140° C. under a nitrogen atmosphere. The mixture was allowed to cool down to room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions (column: XBridge Shield RP18 OBD column: 30*150 mm, 7 m; mobile phase A: water (10 mmol/L NH₄HCO₃+0.05% NH₃H₄O), mobile phase B: ACN; flow rate: 60 mL/min; gradient: 20% B to 45% B in 7 min; wavelength: 254 nm/222 nm; RT1(min): 7; number of runs: 6) to afford 1-[4-(3-fluoro-3-methylazetidin-1-yl)pyridin-2-yl]-N-(1-methylindazol-7-yl)pyrazole-4-sulfonamide (I-440, 63.4 mg, 55.34%) as a white solid. LC-MS (ES, m/z): [M+H]⁺=442; ¹H NMR (DMSO-d₆, 400 MHz): δ 1.65 (d, J=24.0 Hz, 3H), 4.07-4.22 (m, 4H), 4.28 (s, 3H), 6.47 (dd, J=2.0, 8.0 Hz, 1H), 6.69 (dd, J=1.2, 8.0 Hz, 1H), 6.91 (d, J=2.0 Hz, 1H), 6.97 (t, J=7.6 Hz, 1H), 7.68 (d, J=8.0 Hz, 1H), 7.94 (s, 1H), 8.06 (d, J=6.0 Hz, 1H), 8.08 (s, 1H), 8.71 (s, 1H), 9.99 (s, 1H).

### Example 474—Synthesis of 1-(4-(3-Methoxyazetidin-1-yl)Pyridin-2-yl)-N-(1-Methyl-1H-Indazol-7-yl)-1H-Pyrazole-4-Sulfonamide (I-441)

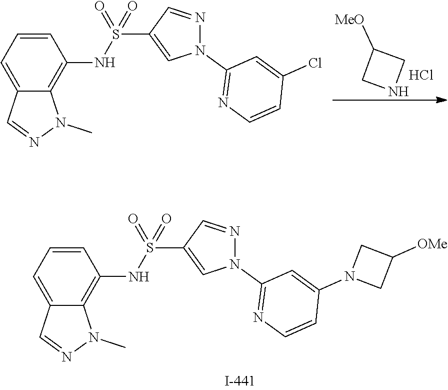 [SMILES: Cn1ncc2cccc(NS(=O)(=O)c3cnn(-c4cc(Cl)ccn4)c3)c21 ; COC1CNC1 ; Cl ; COC1CN(c2ccnc(-n3cc(S(=O)(=O)Nc4cccc5cnn(C)c45)cn3)c2)C1]

A mixture of 1-(4-chloropyridin-2-yl)-N-(1-methyl-1H-indazol-7-yl)-1H-pyrazole-4-sulfonamide (100.00 mg, 0.257 mmol, 1 equiv), 3-methoxyazetidine hydrochloride (63.57 mg, 0.514 mmol, 2 equiv) and Cs₂CO₃ (251.38 mg, 0.771 mmol, 3 equiv) in DMSO (5 mL) was stirred for 16 h at 140° C. The resulting mixture was diluted with water (10 mL) and extracted with EtOAc (3×20 mL). The combined organic layers were washed with brine (1×50 mL) and dried over anhydrous Na₂SO₄. After filtration, the filtrate was concentrated under reduced pressure. The crude product was purified by prep-HPLC with the following conditions (column: ER Prep OBD C18 column: 30*150 mm, 5 μm; mobile phase A: water (10 mmol/L NH₄HCO₃+0.05% NH₃H₂O), mobile phase B: ACN; flow rate: 60 mL/min; gradient: 10% B to 45% B in 8 min; wavelength: 220 nm; RT1(min): 7.62) to afford 1-(4-(3-methoxyazetidin-1-yl)pyridin-2-yl)-N-(1-methyl-1H-indazol-7-yl)-1H-pyrazole-4-sulfonamide (I-441, 59.5 mg, 51.38%) as an off-white solid. LC-MS (ES, m/z): [M+H]⁺=440; ¹H NMR (400 MHz, DMSO-d₆): δ 3.26 (s, 3H), 3.85 (dd, J=3.6, 9.2 Hz, 2H), 4.22 (dd, J=6.4, 9.2 Hz, 2H), 4.28 (s, 3H), 4.43-4.34 (m, 1H), 6.41 (dd, J=2.4, 5.6 Hz, 1H), 6.69 (d, J=7.2 Hz, 1H), 6.86 (d, J=2.0 Hz, 1H), 6.97 (t, J=7.6 Hz, 1H), 7.67 (d, J=8.0 Hz, 1H), 7.93 (s, 1H), 8.03 (d, J=5.6 Hz, 1H), 8.08 (s, 1H), 8.70 (s, 1H), 9.98 (s, 1H).

### Example 475—Synthesis of 1-(4-(3,3-Difluoroazetidin-1-yl)Pyridin-2-yl)-N-(6-Methoxy-1-Methyl-1H-Indazol-7-yl)-1H-Pyrazole-4-Sulfonamide (I-443)

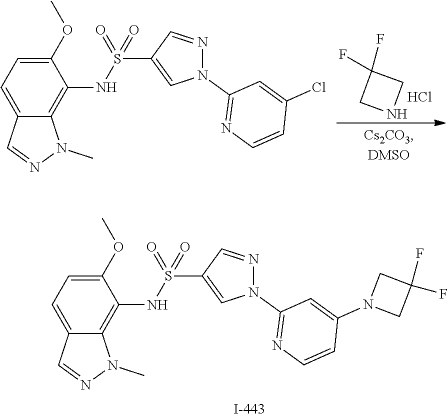

A solution of 1-(4-chloropyridin-2-yl)-N-(6-methoxy-1-methylindazol-7-yl)pyrazole-4-sulfonamide (100 mg, 0.239 mmol, 1 equiv), Cs₂CO₃ (233.36 mg, 0.717 mmol, 3 equiv) and 3,3-difluoroazetidine hydrochloride (61.85 mg, 0.478 mmol, 2 equiv) in DMSO (4 mL) was stirred overnight at 140° C. under a nitrogen atmosphere. The reaction was quenched with water (20 mL) at room temperature. The resulting mixture was extracted with EtOAc (3×20 mL). The combined organic layers were washed with a sat. NaCl solution (aq.) (1×40 mL), and dried over anhydrous Na₂SO₄. After filtration, the filtrate was concentrated under reduced pressure. The crude product (100 mg) was purified by prep-HPLC with the following conditions (column: XBridge Shield RP18 OBD column, 19*250 mm, 5 μm; mobile phase A: water (0.1% FA), mobile phase B: ACN; flow rate: 25 mL/min; gradient: 41% B to 52% B in 9 min; wavelength: 254 nm/220 nm; RT1(min): 8.42; number of runs: 6) to afford 1-[4-(3,3-difluoroazetidin-1-yl)pyridin-2-yl]-N-(6-methoxy-1-methylindazol-7-yl)pyrazole-4-sulfonamide (I-443, 31.7 mg, 27.51%) as a white solid. LC-MS (ES, m/z): [M+1]=476; ¹H NMR (400 MHz, acetonitrile-d3) δ 3.38 (s, 3H), 4.31 (s, 3H), 4.44 (d, J=12.0 Hz, 4H), 6.49 (dd, J=2.4, 5.6 Hz, 1H), 6.81 (d, J=8.8 Hz, 1H), 7.04 (d, J=2.4 Hz, 1H), 7.44 (s, 1H), 7.68 (d, J=8.8 Hz, 1H), 7.80 (d, J=0.8 Hz, 1H), 7.93 (s, 1H), 8.11 (d, J=5.6 Hz, 1H), 8.64 (d, J=0.8 Hz, 1H),

### Example 476—Synthesis of 1-(4-(3-Fluoro-3-Methylazetidin-1-yl)Pyridin-2-yl)-N-(6-Methoxy-1-Methyl-1H-Indazol-7-yl)-1H-Pyrazole-4-Sulfonamide (I-444)

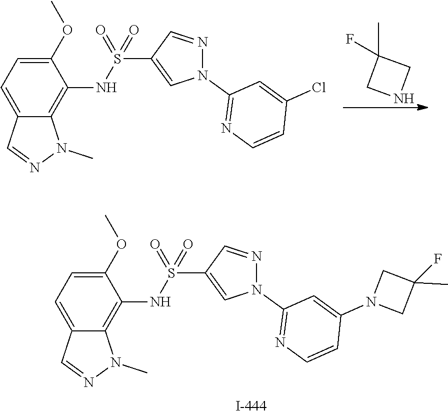

A solution of 1-(4-chloropyridin-2-yl)-N-(6-methoxy-1-methylindazol-7-yl)pyrazole-4-sulfonamide (100 mg, 0.239 mmol, 1 equiv), 3-fluoro-3-methylazetidine (42.55 mg, 0.478 mmol, 2 equiv) and Cs₂CO₃ (233.36 mg, 0.717 mmol, 3 equiv) in DMSO (2 mL) was stirred overnight at 140° C. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (100 mg) was purified by prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 34% B to 54% B in 8 min; wavelength: 220 nm; RT1(min): 7.63) to afford 1-(4-(3-fluoro-3-methylazetidin-1-yl)pyridin-2-yl)-N-(6-methoxy-1-methyl-1H-indazol-7-yl)-1H-pyrazole-4-sulfonamide (I-444, 60.2 mg, 52.78%) as a white solid. LC-MS (ES, m/z): [M+H]⁺=472; ¹H NMR (400 MHz, acetonitrile-d₃): δ 1.69 (d, J=2.4 Hz, 3H), 3.38 (s, 3H), 4.04-4.25 (m, 4H), 4.30 (s, 3H), 6.40 (dd, J=2.4, 5.6 Hz, 1H), 6.81 (d, J=8.8 Hz, 1H), 6.94 (d, J=2.4 Hz, 1H), 7.38 (s, 1H), 7.68 (d, J=8.8 Hz, 1H), 7.79 (d, J=0.8 Hz, 1H), 7.93 (s, 1H), 8.05 (d, J=5.6 Hz, 1H), 8.63 (d, J=0.8 Hz, 1H).

### Example 477—Synthesis of 1-(4-(3-Methoxy-3-Methylazetidin-1-yl)Pyridin-2-yl)-N-(1-Methyl-1H-Indazol-7-yl)-1H-Pyrazole-4-Sulfonamide (I-446)

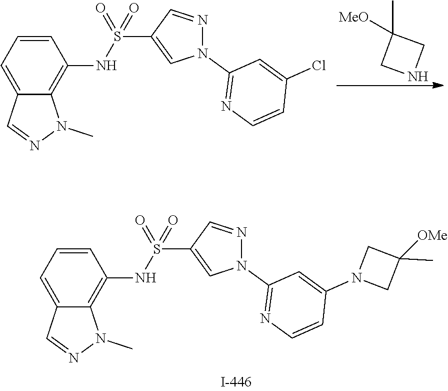

A solution of 1-(4-chloropyridin-2-yl)-N-(1-methylindazol-7-yl)pyrazole-4-sulfonamide (100 mg, 0.257 mmol, 1 equiv) and 3-methoxy-3-methylazetidine (130 mg, 1.285 mmol, 5.00 equiv) and Cs₂CO₃ (250 mg, 0.767 mmol, 2.98 equiv) in DMSO (2 mL) was stirred for 2 h at 100° C. under a nitrogen atmosphere. The resulting mixture was diluted with water (5 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 30% to 50% gradient in 25 min; detector: UV 254 nm. This resulted in 1-[4-(3-methoxy-3-methylazetidin-1-yl)pyridin-2-yl]-N-(1-methylindazol-7-yl)pyrazole-4-sulfonamide (I-446, 50.8 mg, 43.55%) as an off-white solid. LC-MS (ES, m/z): [M+1]⁺=454; ¹H NMR (400 MHz, DMSO-d₆): δ 1.48 (s, 3H), 3.22 (s, 3H), 3.85 (d, J=8.6 Hz, 2H), 3.96 (d, J=8.8 Hz, 2H), 4.28 (s, 3H), 6.42 (dd, J=5.8, 2.2 Hz, 1H), 6.68 (d, J=7.4 Hz, 1H), 6.87 (d, J=2.2 Hz, 1H), 6.97 (t, J=7.8 Hz, 1H), 7.69 (d, J=8.0 Hz, 1H), 7.94 (s, 1H), 8.04 (d, J=5.8 Hz, 1H), 8.09 (s, 1H), 8.71 (s, 1H), 9.99 (s, 1H).

### Example 478—Synthesis of 1-(4-(1-Cyanocyclopentyl)Pyridin-2-yl)-N-(6-Methoxy-1-Methyl-1H-Indazol-7-yl)-1H-Pyrazole-4-Sulfonamide (I-449)

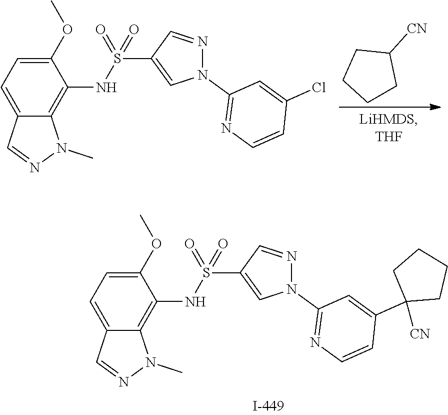

To a solution of 1-(4-chloropyridin-2-yl)-N-(6-methoxy-1-methylindazol-7-yl)pyrazole-4-sulfonamide (100 mg, 0.239 mmol, 1 equiv) and cyclopentanecarbonitrile (45.43 mg, 0.478 mmol, 2 equiv) in THE (1 mL) at 0° C. was added LiHMDS (1.0 M, 0.5 mL, 0.178 mmol, 3 equiv) in THF dropwise. After the addition, the mixture was stirred for 3 hours at 0° C. The resulting mixture was diluted with water (20 mL). The resulting mixture was extracted with CH₂Cl₂ (3×20 mL). The combined organic layers were washed with brine (1×50 mL) and dried over anhydrous Na₂SO₄. After filtration, the filtrate was concentrated under reduced pressure. The crude product (40 mg) was purified by prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 32% B to 57% B in 8 min; wavelength: 220 nm; RT1(min): 7.43) to afford 1-(4-(1-cyanocyclopentyl)pyridin-2-yl)-N-(6-methoxy-1-methyl-1H-indazol-7-yl)-1H-pyrazole-4-sulfonamide (I-449, 8.6 mg, 7.45%) as a white solid. LC-MS (ES, m/z): [M+H]⁺=478; ¹H NMR (400 MHz, methanol-d₄) δ 2.06-2.12 (m, 4H), 2.16-2.24 (m, 2H), 2.43-2.60 (m, 2H), 3.40 (s, 3H), 4.34 (s, 3H), 6.84 (d, J=8.8 Hz, 1H), 7.53 (dd, J=1.6, 5.2 Hz, 1H), 7.67 (d, J=8.8 Hz, 1H), 7.87 (d, J=0.8 Hz, 1H), 7.94 (s, 1H), 8.15 (d, J=2.0 Hz, 1H), 8.45 (d, J=5.2 Hz, 1H), 8.75 (s, 1H).

### Example 479—Synthesis of 1-(4-(1-Cyanocyclopropyl)Pyridin-2-yl)-N-(1-Methyl-1H-Indazol-7-yl)-1H-Pyrazole-4-Sulfonamide (I-469)

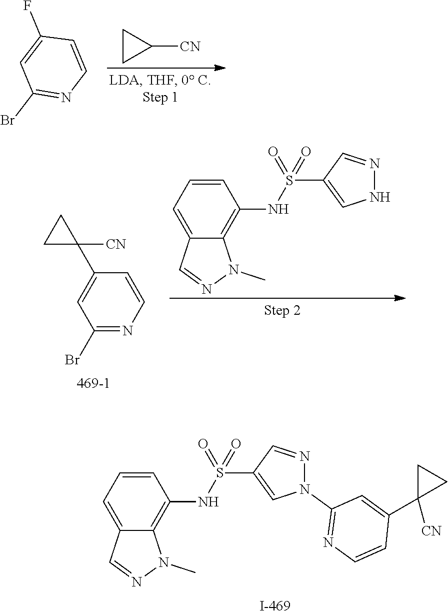

Step 1: Synthesis of 469-1: To a stirred mixture of 2-bromo-4-fluoropyridine (500.00 mg, 2.841 mmol, 1 equiv) and cyclopropanecarbonitrile (285.92 mg, 4.261 mmol, 1.5 equiv) in THF (5 mL) was added LDA (2.13 mL, 4.261 mmol, 1.5 equiv) dropwise at 0° C. under a nitrogen atmosphere. The resulting mixture was stirred for 2 hours at room temperature under a nitrogen atmosphere. The resulting mixture was extracted with EtOAc (3×30 mL). The combined organic layers were washed with brine (1×20 mL) and dried over anhydrous Na₂SO₄. After filtration, the filtrate was concentrated under reduced pressure. The residue was purified by silica gel column chromatography, eluting with PE/EA (5:1) to afford 1-(2-bromopyridin-4-yl)cyclopropane-1-carbonitrile (469-1, 200.00 mg, 30.64%) as an off-white solid.

Step 2: Synthesis of 1-(4-(1-cyanocyclopropyl)pyridin-2-yl)-N-(1-methyl-1H-indazol-7-yl)-1H-pyrazole-4-sulfonamide (I-469): A mixture of 1-(2-bromopyridin-4-yl)cyclopropane-1-carbonitrile (100.00 mg, 0.448 mmol, 1 equiv), N-(1-methyl-1H-indazol-7-yl)-1H-pyrazole-4-sulfonamide (149.17 mg, 0.538 mmol, 1.2 equiv), CuI (42.69 mg, 0.224 mmol, 0.5 equiv), (1S,2S)—N1,N2-dimethylcyclohexane-1,2-diamine (31.88 mg, 0.224 mmol, 0.5 equiv), and Cs₂CO₃ (438.18 mg, 1.344 mmol, 3 equiv) in dioxane (2 mL) was stirred for 16 hours at 90° C. under a nitrogen atmosphere. The resulting mixture was extracted with EtOAc (3×20 mL). The combined organic layers were washed with brine (1×15 mL) and dried over anhydrous Na₂SO₄. After filtration, the filtrate was concentrated under reduced pressure. The crude product was purified by prep-HPLC with the following conditions (column: XBridge Prep OBD C18 column, 30*150 mm, 5 μm; mobile phase A: water (10 mmol/L NH₄HCO₃)+0.05% NH₃·H₂O, mobile phase B: ACN; flow rate: 60 mL/min; gradient: 10% B to 39% B in 7 min; wavelength: 254 nm/220 nm; RT1(min): 7.97) to afford 1-(4-(1-cyanocyclopropyl)pyridin-2-yl)-N-(1-methyl-1H-indazol-7-yl)-1H-pyrazole-4-sulfonamide (I-469, 20.7 mg, 10.95%) as an off-white solid. LC-MS: (ES, m/z): [M+H]⁺=420. ¹H-NMR: (400 MHz, DMSO, ppm) δ 1.85-1.74 (m, 2H), 2.04-1.96 (m, 2H), 4.29 (s, 3H), 6.72 (d, J=7.4 Hz, 1H), 6.96 (t, J=7.8 Hz, 1H), 7.32 (dd, J=5.4, 2.0 Hz, 1H), 7.65 (d, J=8.2 Hz, 1H), 7.91 (d, J=2.0 Hz, 1H), 8.06 (d, J=8.2 Hz, 2H), 8.49 (d, J=5.4 Hz, 1H), 8.79 (s, 1H), 10.06 (s, 1H).

### Example 480—Synthesis of 1-(4-(1-Hydroxy-3,3-Dimethylcyclo-Butyl)Pyridin-2-yl)-N-(1-Methyl-1H-Indazol-7-yl)-1H-Pyrazole-4-Sulfonamide (I-477)

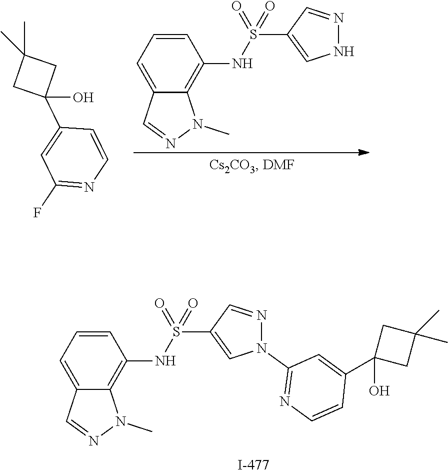 [SMILES: CC1(C)CC(O)(c2ccnc(F)c2)C1 ; Cn1ncc2cccc(NS(=O)(=O)c3cn[nH]c3)c21 ; Cn1ncc2cccc(NS(=O)(=O)c3cnn(-c4cc(C5(O)CC(C)(C)C5)ccn4)c3)c21]

To a stirred mixture of 1-(2-fluoropyridin-4-yl)-3,3-dimethylcyclobutan-1-ol (100 mg, 0.512 mmol, 1 equiv) and N-(1-methylindazol-7-yl)-1H-pyrazole-4-sulfonamide (113.63 mg, 0.410 mmol, 0.8 equiv) in DMF (5 mL) was added Cs₂CO₃ (500.65 mg, 1.536 mmol, 3 equiv) in portions at 80° C. under air atmosphere. The resulting mixture was stirred overnight at 80° C. The crude product (100 mg) was purified by prep-HPLC with the following conditions (column: XBridge Prep OBD C18 column, 30*150 mm, 5 μm; mobile phase A: water (10 mmol/L NH₄HCO₄+0.5% NH₃H₂O), mobile phase B: ACN; flow rate: 60 mL/min; gradient: 20% B to 41% B in 7 min; wavelength: 254/220 nm; RT1(min): 7.05) to afford 1-(4-(1-hydroxy-3,3-dimethylcyclobutyl)pyridin-2-yl)-N-(1-methyl-1H-indazol-7-yl)-1H-pyrazole-4-sulfonamide (I-477, 11.8 mg, 5.06%) as a white solid. LC-MS: (ES, m/z): [M+1]=453. ¹H-NMR: (400 MHz, Chloroform-d) 61.19 (t, J=4.2 Hz, 4H), 1.41 (dd, J=8.2, 4.4 Hz, 3H), 2.26 (d, J=12.2 Hz, 2H), 2.46 (d, J=12.8 Hz, 2H), 4.42 (dd, J=7.8, 4.0 Hz, 3H), 6.33 (s, 1H), 6.78 (s, 1H), 6.96 (s, 1H), 7.39 (s, 1H), 7.70 (s, 1H), 7.79 (d, J=5.6 Hz, 1H), 8.02 (s, 1H), 8.09 (s, 1H), 8.40 (s, 1H), 8.88 (s, 1H).

### Example 481—Synthesis of 1-(4-(1-Methoxy-3,3-Dimethylcyclobutyl) Pyridin-2-yl)-N-(1-Methyl-1H-Indazol-7-yl)-1H-Pyrazole-4-Sulfonamide (I-478)

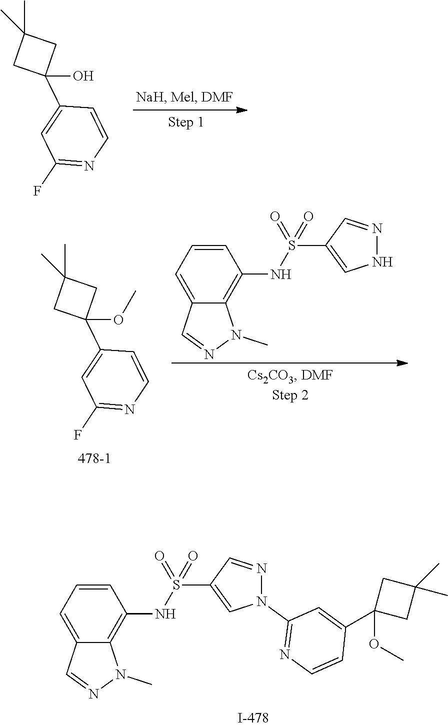

Step 1: Synthesis of 478-1: To a stirred mixture of 1-(2-fluoropyridin-4-yl)-3,3-dimethylcyclobutan-1-ol (400 mg, 2.04 mmol, 1.00 equiv) in DMF (8 mL) was added NaH (122 mg, 5.12 mmol, 2.50 equiv) at 0° C. under a nitrogen atmosphere. The resulting mixture was stirred for 30 minutes at room temperature under a nitrogen atmosphere. To the resulting mixture was then added Mel (436 mg, 3.07 mmol, 1.50 equiv) dropwise at 0° C. The resulting mixture was stirred for an additional 1 hour at room temperature. The reaction was quenched with water at room temperature. The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% NH₃·H₂O), 40% to 60% gradient in 10 min; detector, UV 254 nm. The product, 2-fluoro-4-(1-methoxy-3,3-dimethylcyclobutyl)pyridine (478-1, 140 mg, 32%), was obtained as a yellow oil. LC-MS: 210 [M+H]⁺.

Step 2: Synthesis of 1-(4-(1-methoxy-3,3-dimethylcyclobutyl)pyridin-2-yl)-N-(1-methyl-1H-indazol-7-yl)-1H-pyrazole-4-sulfonamide (I-478): A mixture of 2-fluoro-4-(1-methoxy-3,3-dimethylcyclobutyl) pyridine (70 mg, 0.335 mmol, 1.00 equiv), N-(1-methylindazol-7-yl)-1H-pyrazole-4-sulfonamide (139 mg, 0.503 mmol, 1.50 equiv), and Cs₂CO₃ (326 mg, 1.00 mmol, 3.00 equiv) in DMF (5 mL) was stirred overnight at 80° C. under a nitrogen atmosphere. The reaction was quenched with water at room temperature. The resulting mixture was extracted with EtOAc (3×5 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 40% to 60% gradient in 10 min; detector, UV 254 nm. The product, 1-[4-(1-methoxy-3,3-dimethylcyclobutyl)pyridin-2-yl]-N-(1-methylindazol-7-yl)pyrazole-4-sulfonamide (I-478, 73.9 mg, 47%), was obtained as an off-white solid. LC-MS: 467 [M+H]⁺. ¹H NMR (400 MHz, Chloroform-d) 61.09 (s, 3H), 1.32 (s, 3H), 2.29 (s, 4H), 2.98 (s, 3H), 4.42 (s, 3H), 6.80 (dd, J=7.3, 1.0 Hz, 1H), 6.97 (dd, J=8.1, 7.3 Hz, 1H), 7.31 (dd, J=5.1, 1.5 Hz, 1H), 7.71 (dd, J=8.0, 1.0 Hz, 1H), 7.80 (d, J=0.8 Hz, 1H), 8.03-7.97 (m, 2H), 8.41 (dd, J=5.1, 0.8 Hz, 1H), 8.89 (d, J=0.8 Hz, 1H).

### Example 482—Synthesis of 1-(4-(1-Methoxy-2-Methylpropan-2-yl)Pyridin-2-yl)-N-(1-Methyl-1H-Indazol-7-yl)-1H-Pyrazole-4-Sulfonamide (I-481)

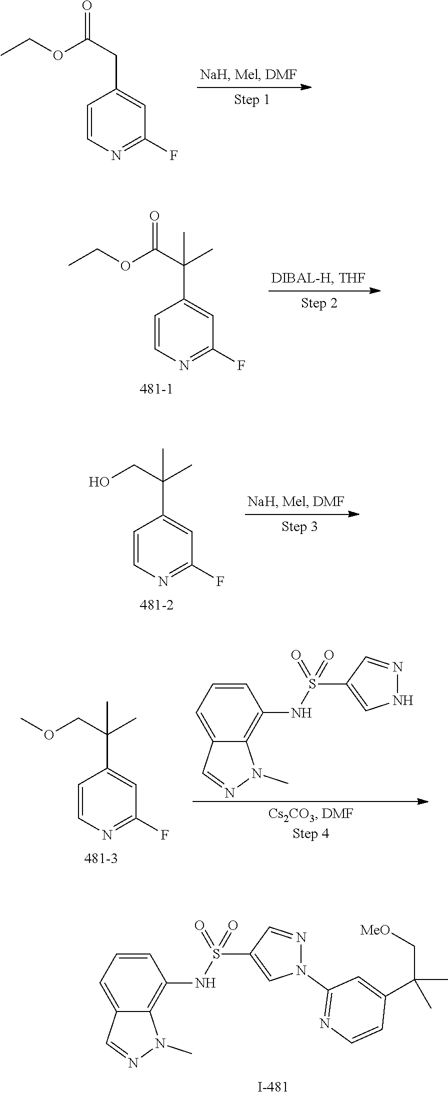 [SMILES: CCOC(=O)Cc1ccnc(F)c1 ; CCOC(=O)C(C)(C)c1ccnc(F)c1 ; CC(C)(CO)c1ccnc(F)c1 ; COCC(C)(C)c1ccnc(F)c1 ; Cn1ncc2cccc(NS(=O)(=O)c3cn[nH]c3)c21 ; COCC(C)(C)c1ccnc(-n2cc(S(=O)(=O)Nc3cccc4cnn(C)c34)cn2)c1]

Step 1: Synthesis of 481-1: A solution of ethyl 2-(2-fluoropyridin-4-yl)acetate (900 mg, 4.913 mmol, 1 equiv) in DMF (20 mL) was treated with NaH (600 mg, 25.002 mmol, 5.09 equiv) for 30 minutes at 0° C. under a nitrogen atmosphere, after which Mel (3.49 g, 24.588 mmol, 5.00 equiv) was added dropwise at 0° C. The resulting mixture was stirred for 2 hours at room temperature under a nitrogen atmosphere. The reaction was quenched with saturated aqueous NH₄Cl at 0° C. The resulting mixture was extracted with EtOAc (3×100 mL). The combined organic layers were washed with brine (1×5 mL) and dried over anhydrous Na₂SO₄. After filtration, the filtrate was concentrated under reduced pressure. The product 481-1 (250 mg, 24.09%) was obtained as a yellow oil.

Step 2: Synthesis of 481-2: To a stirred mixture of compound 481-1 (300 mg, 1.420 mmol, 1 equiv) in THE (100 mL) was added diisobutylaluminium hydride (605.94 mg, 4.260 mmol, 3 equiv) in portions at 0° C. under air atmosphere. The resulting mixture was stirred for 2 hours at 0° C. The reaction was quenched with saturated aqueous NH₄Cl at 0° C. The resulting mixture was extracted with CH₂Cl₂ (3×20 mL). The combined organic layers were washed with brine (2×5 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. The product 481-2 (200 mg, 83.23%) was obtained as a yellow oil.

Step 3: Synthesis of 481-3: To a stirred mixture of compound 481-2 (200 mg, 1.182 mmol, 1 equiv) and NaH (85.10 mg, 3.546 mmol, 3 equiv) in DMF (10 mL) was added CH₃I (251.67 mg, 1.773 mmol, 1.5 equiv) in portions at 0° C. under air atmosphere. The resulting mixture was stirred for 2 hours at 0° C. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. The product 481-3 (130 mg, 60.02%) was obtained as a yellow oil.

Step 4: Synthesis of 1-(4-(1-methoxy-2-methylpropan-2-yl)pyridin-2-yl)-N-(1-methyl-1H-indazol-7-yl)-1H-pyrazole-4-sulfonamide (I-481): To a stirred mixture of compound 481-3 (50 mg, 0.273 mmol, 1 equiv) and N-(1-methylindazol-7-yl)-1H-pyrazole-4-sulfonamide (75.67 mg, 0.273 mmol, 1 equiv) in DMF (3 mL) was added Cs₂CO₃ (266.74 mg, 0.819 mmol, 3 equiv) in portions at 100° C. under air atmosphere. The resulting mixture was stirred overnight at 100° C. The crude product (60 mg) was purified by prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 19% B to 44% B in 8 min; wavelength: 220 nm; RT1(min): 8.52) to afford 1-(4-(1-methoxy-2-methylpropan-2-yl)pyridin-2-yl)-N-(1-methyl-1H-indazol-7-yl)-1H-pyrazole-4-sulfonamide (I-481, 14 mg, 11.60%) as a white solid. LC-MS (ES, m/z): [M+1]=441. ¹H-NMR: H NMR (400 MHz, chloroform-d) 1.38 (s, 6H), 3.33 (s, 3H), 3.47 (s, 2H), 4.43 (s, 3H), 6.33 (s, 1H), 6.77 (d, J=7.4 Hz, 1H), 6.97 (t, J=7.6 Hz, 1H), 7.31 (dd, J=5.4, 1.8 Hz, 1H), 7.67-7.74 (m, 1H), 7.76-7.81 (m, 1H), 7.97-8.04 (m, 2H), 8.33 (d, J=5.4 Hz, 1H), 8.89 (d, J=0.8 Hz, 1H).

### Example 483—Synthesis of 1-(4-(5-Methoxyspiro[2.3]Hexan-5-yl)Pyridin-2-yl)-N-(1-Methyl-1H-Indazol-7-yl)-1H-Pyrazole-4-Sulfonamide (I-484)

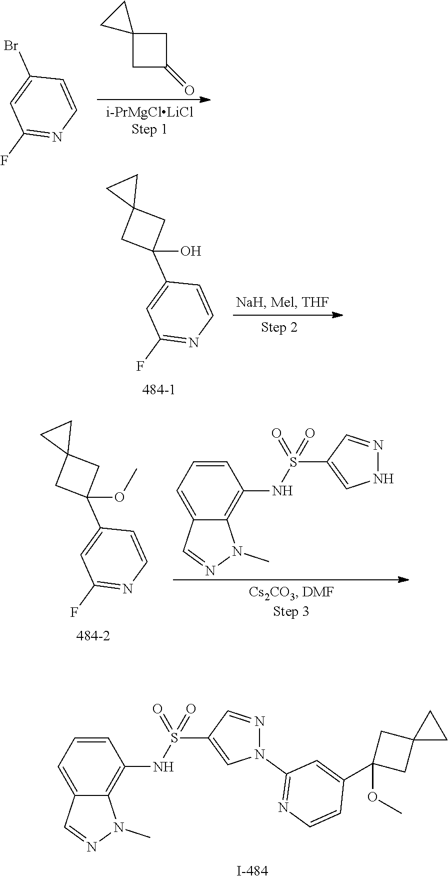

Step 1: Synthesis of 484-1: A solution of 4-bromo-2-fluoropyridine (1 g, 5.682 mmol, 1 equiv) in THF (30 mL) was treated with butyllithium (6.82 mL, 17.046 mmol, 3 equiv) for 1 hour at −78° C. under a nitrogen atmosphere, after which spiro[2.3]hexan-5-one (1.64 g, 17.046 mmol, 3.00 equiv) was added dropwise at −78° C. The resulting mixture was stirred for 2 hours at −78° C. under a nitrogen atmosphere. The desired product could be detected by LCMS. The reaction was quenched with saturated aqueous HCl at 0° C. The resulting mixture was extracted with EtOAc (2×300 mL). The combined organic layers were washed with brine (1×100 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. The product, 5-(2-fluoropyridin-4-yl)spiro[2.3]hexan-5-ol (484-1, 650 mg, 54.47%), was obtained as a light-yellow solid.

Step 2: Synthesis of 484-2: A solution of 5-(2-fluoropyridin-4-yl)spiro[2.3]hexan-5-ol (200 mg, 1.035 mmol, 1 equiv) in THE (4 mL) was treated with NaH (37.26 mg, 1.552 mmol, 1.5 equiv) for 30 min at 0° C. under a nitrogen atmosphere, after which Mel (293.84 mg, 2.070 mmol, 2 equiv) was added dropwise at 0° C. The resulting mixture was stirred for 2 hours at room temperature under a nitrogen atmosphere. The desired product could be detected by LCMS. The reaction was quenched with ice at 0° C. The resulting mixture was extracted with EtOAc (2×100 mL). The combined organic layers were washed with brine (1×100 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm. The product, 2-fluoro-4-{5-methoxyspiro[2.3]hexan-5-yl}pyridine (484-2, 175 mg, 75.87%), was obtained as a light-yellow oil.

Step 3: Synthesis of 1-(4-(5-methoxyspiro[2.3]hexan-5-yl)pyridin-2-yl)-N-(1-methyl-1H-indazol-7-yl)-1H-pyrazole-4-sulfonamide (I-484): A solution of 2-fluoro-4-{5-methoxyspiro [2.3] hexan-5-yl} pyridine (60 mg, 0.290 mmol, 1 equiv) in DMF (3 mL) was treated with N-(1-methylindazol-7-yl)-1H-pyrazole-4-sulfonamide (96.34 mg, 0.348 mmol, 1.2 equiv) followed by the addition of t-BuOK (97.46 mg, 0.870 mmol, 3 equiv). The resulting mixture was stirred for 2 hours at 100° C. under a nitrogen atmosphere. The desired product could be detected by LCMS. The reaction was quenched with water at room temperature. The resulting mixture was extracted with EtOAc (2×80 mL). The combined organic layers were washed with brine (2×50 mL) and dried over anhydrous Na₂SO₄. After filtration, the filtrate was concentrated under reduced pressure. The crude product (80 mg) was purified by prep-HPLC with the following conditions (column: XBridge Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 23% B to 48% B in 8 min; wavelength: 220 nm; RT1(min): 8.83) to afford 1-(4-{5-methoxyspiro [2.3] hexan-5-yl}pyridin-2-yl)-N-(1-methylindazol-7-yl)pyrazole-4-sulfonamide (I-484, 54.1 mg, 39.22%) as an off-white solid. LC-MS: (ES, m/z): [M+H]⁺=465. ¹H-NMR: (400 MHz, DMSO, ppm) δ 0.55 (s, 4H), 2.29-2.37 (m, 2H), 2.63-2.71 (m, 2H), 3.05 (s, 3H), 4.30 (s, 3H), 6.74 (m, J=7.4, 1.0 Hz, 1H), 6.97 (t, J=7.8 Hz, 1H), 7.59 (m, J=5.2, 1.6 Hz, 1H), 7.66 (d, J=8.0 Hz, 1H), 8.02-8.10 (m, 3H), 8.56 (d, J=5.2 Hz, 1H), 8.82 (s, 1H), 10.05 (s, 1H).

### Example 484—Synthesis of Additional Compounds

Compounds in Table 3 below were prepared based on procedures described herein above.

TABLE 3

| Compound | | |
|---|---|---|
| No. | Structure | Spectroscopic Data |
| I-18 | 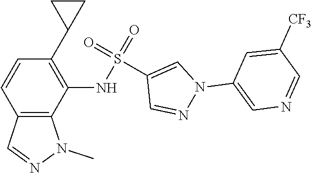 [SMILES: Cn1ncc2ccc(C3CC3)c(NS(=O)(=O)c3cnn(-c4cncc(C(F)(F)F)c4)c3)c21] | |
| I-92 | 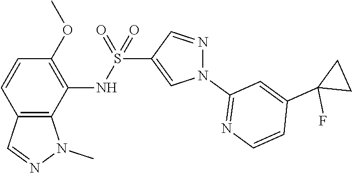 [SMILES: COc1ccc2cnn(C)c2c1NS(=O)(=O)c1cnn(-c2cc(C3(F)CC3)ccn2)c1] | |
| I-239 | 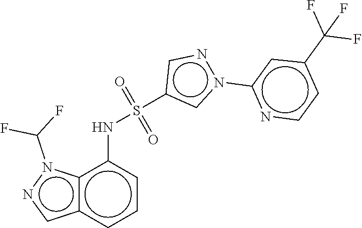 | LC-MS (ES, m/z): [M +1]⁺ = 459. ¹H NMR (400 MHz, DMSO-d₆): δ 6.97 (d, J = 7.6 Hz, 1H), 7.14 (t, J = 8.0 Hz, 1H), 7.67 (s, 1H), 7.86 (d, J = 5.2 Hz, 1H), 8.06 (d, J = 2.4 Hz, 1H), 8.16 (s, 1H), 8.47 (s, 1H), 8.78 (d, J = 16.4 Hz, 1H), 8.80 (s, 1H), 8.84 (s, 1H), 10.38 (s, 1H). |
| I-241 | 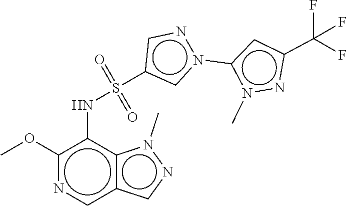 | LC-MS (ES, m/z): [M + 1]⁺ = 457. ¹H NMR (400 MHz, DMSO-d₆): δ 3.55(s, 3H), 3.89(s, 3H), 4.21(s, 3H), 7.17(s, 1H), 8.10(s, 1H), 8.21 (s, 1H), 8.66(s, 1H), 8.69(s, 1H), 9.93(s, 1H). |
| I-242 | 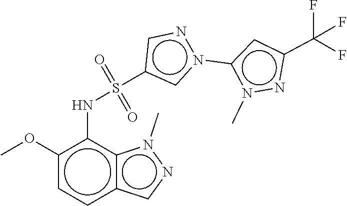 | LC-MS (ES, m/z): [M + 1]⁺ = 456. ¹H NMR (400 MHz, DMSO-d₆): δ 3.46 (s, 3H), 3.90 (s, 3H), 4.24 (s, 3H), 6.92 (d, J = 8.8 Hz, 1H), 7.18 (s, 1H), 7.68 (d, J = 8.8 Hz, 1H), 7.97 (s, 1H), 8.07 (s, 1H), 8.65 (s, 1H), 9.84 (s, 1H). |
| I-243 | 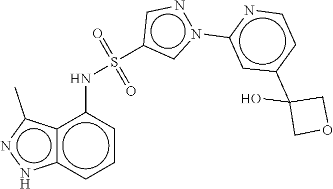 | LC-MS (ES, m/z): [M + 1]⁺ = 427. ¹H NMR (400 MHz, DMSO-d₆): δ 2.59 (s, 3H), 4.68 (d, J = 6.8 Hz, 2H), 4.84 (d, J = 6.8 Hz, 2H), 6.56 (d, J = 7.2 Hz, 1H), 6.84 (s, 1H), 7.17 (d, J = 7.6 Hz, 1H), 7.29 (s, 1H), 7.70 (d, J = 4.8 Hz, 1H), 8.01 (s, 1H), 8.18 (s, 1H), 8.53 (d, J = 5.2 Hz, 1H), 8.79 (s, 1H), 9.91 (s, 1H), 12.68 (s, 1H). |
| I-245 | 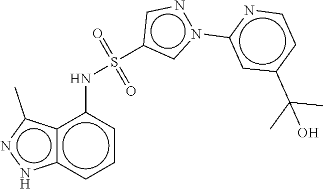 | LC-MS (ES, m/z): [M + H]⁺ = 413. ¹H NMR (400 MHz, DMSO-d₆): δ 1.46 (s, 6H), 2.59 (s, 3H), 5.47 (s, 1H), 6.55 (d, J = 7.2 Hz, 1H), 7.18 (t, J = 8.0 Hz, 1H), 7.34 (d, J = 8.4 Hz, 1H), 7.51 (d, J = 5.2 Hz, 1H), 7.99 (s, 1H), 8.07 (s, 1H), 8.42 (d, J = 5.2 Hz, 1H), 8.78 (s, 1H), 9.89 (s, 1H), 12.73 (s, 1H). |
| I-252 | 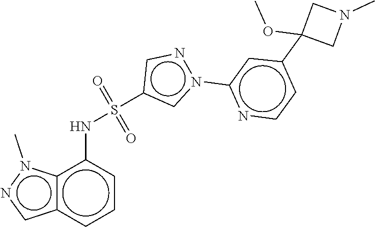 | LC-MS (ES, m/z): [M + 1]⁺ = 454. ¹H NMR (400 MHz, DMSO-d₆): δ 2.38 (s, 3H), 3.09 (s, 3H), 3.32 (d, J = 8.4 Hz, 2H), 3.59 (d, J = 8.0 Hz, 2H), 4.30 (s, 3H), 6.73 (d, J = 7.2 Hz, 1H), 6.96 (t, J = 7.6 Hz, 1H), 7.58 (dd, J = 1.6, 5.2 Hz, 1H), 7.64 (d, J = 8.0 Hz, 1H), 8.03 (s, 1H), 8.07 (s, 1H), 8.13 (s, 1H), 8.54 (d, J = 4.8 Hz, 1H), 8.81 (s, 1H), 10.05 (br s, 1H). |
| I-254 | 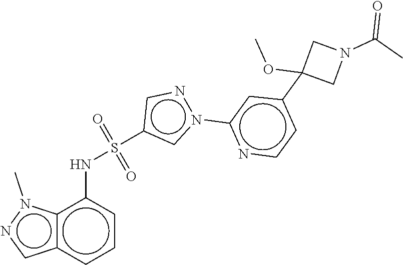 | LC-MS (ES, m/z): [M + 1]⁺ = 482. ¹H NMR (400 MHz, DMSO-d₆): δ 1.86 (s, 3H), 3.16 (s, 3H), 4.03- 4.18 (m, 2H), 4.31 (s, 3H), 4.43 (s, 2H), 6.74 (d, J = 7.6 Hz, 1H), 6.93 (t, J = 7.6 Hz, 1H), 7.50 (d, J = 1.2 Hz, 1H), 7.62 (d, J = 1.2 Hz, 1H), 7.97 (s, 1H), 8.03 (d, J = 1.6 Hz, 2H), 8.58 (d, J = 5.2 Hz, 1H), 8.79 (s, 1H). |
| I-267 | 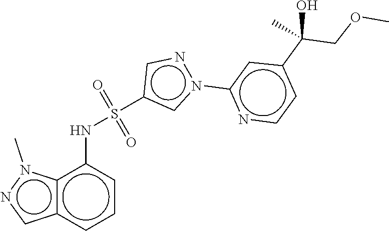 | LC-MS (ES, m/z): [M + 1]⁺ = 443. ¹H NMR (400 MHz, DMSO-d₆): δ 1.44 (s, 3H), 3.43 (d, J = 9.4 Hz, 3H), 3.44 (d, J = 9.4 Hz, 1H), 3.53 (d, J = 9.4 Hz, 1H), 4.29 (s, 3H), 5.60 (s, 1H), 6.70 (d, J = 7.2 Hz, 1H), 6.94-7.03 (m, 1H), 7.51 (dd, J = 5.2, 1.6 Hz, 1H), 7.71 (dd, J = 8.2, 1.0 Hz, 1H), 8.02 (d, J = 0.8 Hz, 1H), 8.10 (d, J = 1.8 Hz, 1H), 8.43 (d, J = 5.2 Hz, 1H), 8.80 (d, J = 0.8 Hz, 1H), 10.03 (s, 1H). |
| I-268 | 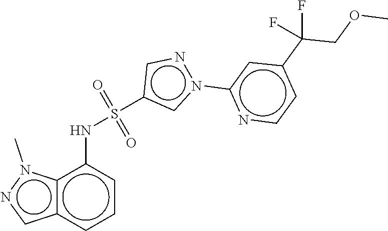 | LC-MS (ES, m/z): [M + 1]⁺ = 449. ¹H NMR (400 MHz, DMSO-d₆): 83.34 (s, 3H), 3.89-3.96 (t, J = 12.4 Hz, 2H), 4.39 (s, 3H), 6.83 (dd, J = 7.4, 2.2 Hz, 1H), 6.94 (t, J = 7.7 Hz, 1H), 7.57-7.47 (m, 2H), 7.88 (s, 1H), 7.95 (d, J = 1.7 Hz, 1H), 8.11 (s, 1H), 8.56 (d, J = 5.1 Hz, 1H), 8.79 (s, 1H). |
| I-269 | 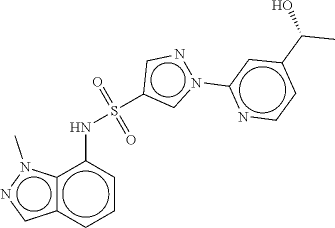 | LC-MS (ES, m/z): [M + 1]⁺ = 399. ¹H NMR (400 MHz, DMSO-d₆): δ 1.38 (d, J = 6.4 Hz, 3H), 4.28 (s, 3H), 4.82-4.94(m, 1H), 5.62 (d, J = 4.4 Hz, 1H), 6.70 (d, J = 7.2 Hz, 1H), 6.97 (t, J = 7.6 Hz, 1H), 7.40- 7.48 (m, 1H), 7.69 (d, J = 8.0 Hz, 1H), 8.00 (d, J = 6.0 Hz, 2H), 8.09 (s, 1H), 8.43 (d, J = 5.2 Hz, 1H), 8.78 (s, 1H), 10.03 (s, 1H). |
| I-270 | 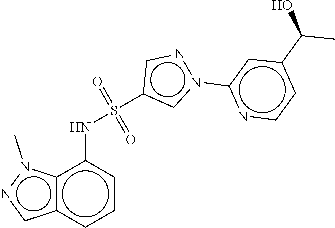 | LC-MS (ES, m/z): [M + 1]⁺ = 399. ¹H NMR (400 MHz, DMSO-d₆): δ 1.38 (d, J = 6.4 Hz, 3H), 4.28 (s, 3H), 4.83-4.89 (m, 1H), 5.62 (d, J = 4.4 Hz, 1H), 6.70 (d, J = 7.2 Hz, 1H), 6.98 (t, J = 7.6 Hz, 1H), 7.43 (dd, J = 5.2, 1.6 Hz, 1H), 7.70 (dd, J = 8.0, 0.8 Hz, 1H), 8.01 (d, J = 5.6 Hz, 2H), 8.09 (s, 1H), 8.43 (d, J = 5.2 Hz, 1H), 8.79 (s, 1H), 10.02 (s, 1H). |
| I-275 | 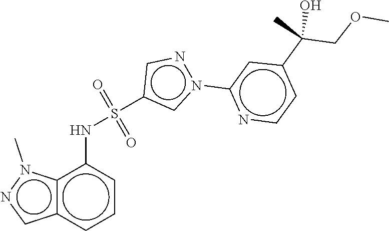 | LC-MS (ES, m/z): [M + 1]⁺ = 443. ¹H NMR (400 MHz, DMSO-d₆): δ 1.44 (s, 3H), 3.24 (s, 3H), 3.43 (d, J = 9.6 Hz, 1H), 3.53 (d, J = 9.6 Hz, 1H), 4.29 (s, 3H), 6.70 (d, J = 7.4 Hz, 1H), 7.01-6.95 (m, 1H), 7.51 (dd, J = 5.2, 1.6 Hz, 1H), 7.71 (dd, J = 8.2, 1.0 Hz, 1H), 8.02 (d, J = 0.8 Hz, 1H), 8.10 (d, J = 1.8 Hz, 2H), 8.43 (d, J = 5.2 Hz, 1H), 8.80 (d, J = 0.8 Hz, 1H), 10.02 (s, 1H). |
| I-276 | 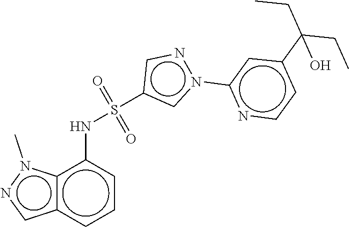 | LC-MS (ES, m/z): [M + H]⁺ = 441. ¹H NMR (400 MHz, DMSO-d₆): δ 0.67 (t, J = 7.2 Hz, 6H), 1.62-1.88 (m, 4H), 4.29 (s, 3H), 5.03 (s, 1H), 6.73 (dd, J = 1.2, 7.2 Hz, 1H), 6.98 (t, J = 7.6 Hz, 1H), 7.42 (dd, J = 1.2, 5.2 Hz, 1H), 7.69 (d, J = 8.4 Hz, 1H), 8.00 (s, 1H), 8.03 (s, 1H), 8.08 (s, 1H), 8.43 (d, J = 5.2 Hz, 1H), 8.80 (s, 1H), 10.03 (s, 1H). |
| I-277 | 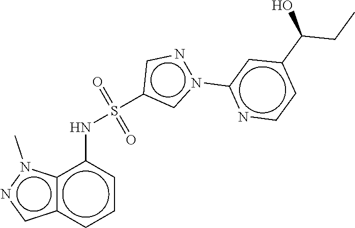 | LC-MS (ES, m/z): [M + H]⁺ = 412. ¹H NMR (400 MHz, DMSO-d₆): δ 0.87 (t, J = 7.2 Hz, 3H), 1.71-1.99 (m, 2H), 4.28 (s, 3H), 4.61-4.68 (t, J = 5.2 Hz, 1 H), 5.56 (s, 1H), 6.71 (d, J = 7.2 Hz, 1H), 6.98 (t, J = 8.0 Hz, 1H), 7.40 (d, J = 5.2 Hz, 1H), 7.71 (d, J = 8.0 Hz, 1H), 8.00 (d, J = 8.8 Hz, 1H), 8.05 (s, 1H), 8.09 (s, 1H), 8.43 (d, J = 5.2 Hz, 1H), 8.79 (s, 1H), 10.03 (s, 1H). |
| I-283 | 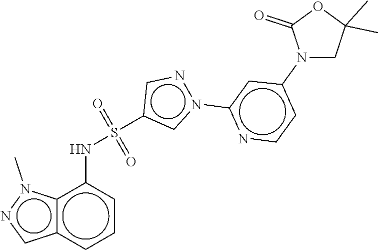 | LC-MS (ES, m/z): [M + H]⁺ = 468.15. ¹H NMR (400 MHz, DMSO-d₆): δ 1.51 (s, 6H), 3.97 (s, 2H), 4.28 (s, 3H), 6.70 (dd, J = 7.4, 1.2 Hz, 1H), 6.94-7.02 (m, 1H), 7.50 (dd, J = 5.8, 2.4 Hz, 1H), 7.71 (dd, J = 8.0, 1.0 Hz, 1H), 8.03 (d, J = 1.2 Hz, 1H), 8.09 (s, 1H), 8.28 (d, J = 2.4 Hz, 1H), 8.43 (d, J = 5.8 Hz, 1H), 8.79 (d, J = 1.2 Hz, 1H), 10.04 (s, 1H). |
| I-284 | 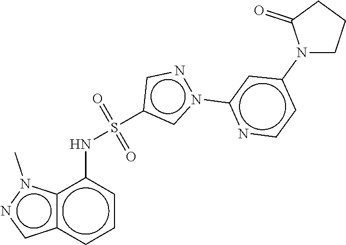 | LC-MS (ES, m/z): [M + H]⁺ = 438. ¹H NMR (400 MHz, DMSO-d₆): δ 2.11 (d, J = 6.8 Hz, 2H), 2.61 (t, J = 8.4 Hz, 2H), 3.93 (t, J = 7.4 Hz, 2H), 4.28 (s, 3H), 6.71 (dd, J = 7.4, 1.0 Hz, 1H), 6.98 (t, J = 7.8 Hz, 1H), 7.61 (dd, J = 5.8, 2.4 Hz, 1H), 7.70 (dd, J = 8.4, 1.0 Hz, 1H), 8.03 (d, J = 0.8 Hz, 1H), 8.09 (s, 1H), 8.43 (d, J = 5.6 Hz, 1H), 8.48 (d, J = 2.0 Hz, 1H), 8.78 (d, J = 0.8 Hz, 1H), 10.03 (s, 1H). |
| I-287 | 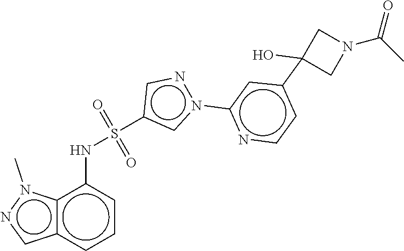 | LC-MS (ES, m/z): [M + 1]⁺ = 468. ¹H NMR (400 MHz, DMSO-d₆): δ 1.87 (s, 3H), 4.02-4.09 (m, 2H), 4.30 (d, J = 9.6 Hz, 4H), 4.40 (s, 1H), 6.74 (d, J = 7.2 Hz, 1H), 6.84 (s, 1H), 6.91 (t, J = 7.6 Hz, 1H), 7.51 (s, 1H), 7.57-7.60 (m, 1H), 8.00 (s, 1H), 8.07 (d, J = 7.6 Hz, 1H), 8.15 (s, 1H), 8.51 (d, J = 5.2 Hz, 1H), 8.76 (s, 1H), 10.20 (s, 1H). |
| I-289 | 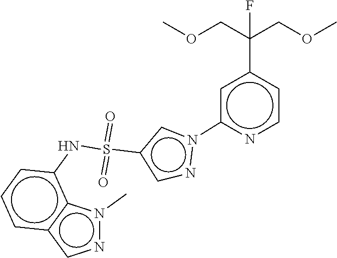 | LC-MS (ES, m/z): [M + H]⁺ = 475. ¹H NMR (400 MHz, DMSO-d₆): δ 3.78-3.90 (m, 4H), 3.27 (s, 6H), 4.30 (s, 3H), 6.72 (d, J = 7.4 Hz, 1H), 6.97 (d, J = 7.6 Hz, 1H), 7.50 (d, J = 5.2 Hz, 1H), 7.66 (s, 1H), 7.98 (s, 1H), 8.03 (s, 1H), 8.07 (s, 1H), 8.53 (d, J = 5.2 Hz, 1H), 8.80 (s, 1H), 10.04 (s, 1H). |
| I-294 | 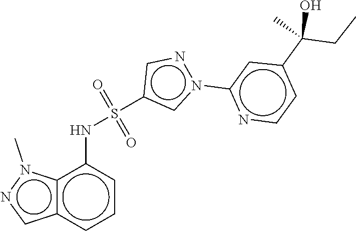 | LC-MS (ES, m/z): [M + 1]⁺ = 427. ¹H NMR (400 MHz, DMSO-d₆): δ 0.71 (t, J = 7.2 Hz, 3H), 1.45 (s, 3H), 1.72-1.80 (m, 2H), 4.29 (s, 3H), 5.31 (s, 1H), 6.71 (d, J = 7.2 Hz, 1H), 6.98 (t, J = 7.6 Hz, 1H), 7.47 (s, 1H), 7.70 (d, J = 8.0 Hz, 1H), 8.01 (s, 1H), 8.04 (s, 1H), 8.12 (dd, J = 5.2, 8.0 Hz, 1H), 8.43 (d, J = 5.2 Hz, 1H), 8.80 (s, 1H), 10.03 (s, 1H). |
| I-295 | 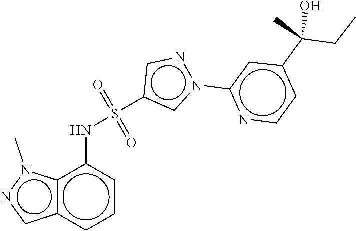 | LC-MS (ES, m/z): [M + 1]⁺ = 427. ¹H NMR (400 MHz, DMSO-d₆): δ 0.71 (t, J = 7.4 Hz, 3H), 1.45 (s, 3H), 1.77 (q, J = 6.9 Hz, 2H), 4.29 (s, 3H), 6.71 (d, J = 7.2 Hz, 1H), 6.98 (t, J = 7.6 Hz, 1H), 7.47 (d, J = 5.2 Hz, 1H), 7.71 (d, J = 8.0 Hz, 1H), 8.01 (s, 1H), 8.04 (s, 1H), 8.12 (dd, J = 5.2, 8.0 Hz, 1H), 8.43 (d, J = 5.2 Hz, 1H), 8.80 (s, 1H), 10.02 (s, 1H). |
| I-296 | 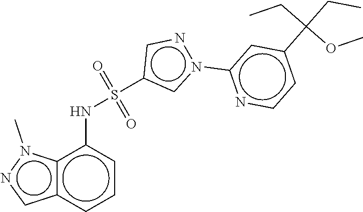 | LC-MS (ES, m/z): [M + 1]⁺ = 455. ¹H NMR (400 MHz, DMSO-d₆): δ 0.57 (t, J = 7.2 Hz, 6H), 1.78 (q, J = 7.2 Hz, 2H), 1.99 (q, J = 7.2 Hz, 2H), 3.17 (s, 3H), 4.30 (s, 3H), 6.74 (dd, J = 1.2, 7.2 Hz, 1H), 6.98 (t, J = 7.6 Hz, 1H), 7.44 (dd, J = 1.6, 5.2 Hz, 1H), 7.68 (d, J = 8.0 Hz, 1H), 7.99 (d, J = 1.6 Hz, 1H), 8.01 (s, 1H), 8.08 (s, 1H), 8.47 (d, J = 5.2 Hz, 1H), 8.81 (s, 1H), 10.04 (s, 1H). |
| I-298 | 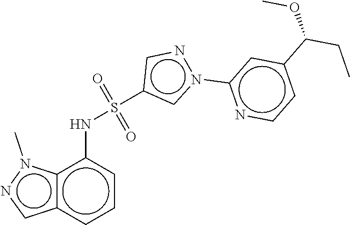 | LC-MS (ES, m/z): [M + 1]⁺ = 427. ¹H NMR (400 MHz, DMSO-d₆): δ 0.84 (t, J = 7.6 Hz, 3H), 1.71 (m, 2H), 3.23 (s, 3H), 4.29 (s, 3H), 4.33 (q, J = 6.4 Hz, 1H), 6.72 (d, J = 7.2 Hz, 1H), 6.99 (t, J = 7.2 Hz, 1H), 7.40 (dd, J = 1.6, 5.2 Hz, 1H), 7.71 (d, J = 8.0 Hz, 1H), 7.91 (s, 1H), 8.03 (s, 1H), 8.10 (s, 1H), 8.49 (d, J = 5.2 Hz, 1H), 8.81 (s, 1H), 10.04 (s, 1H). |
| I-300 | 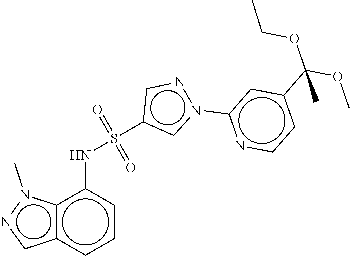 | LC-MS (ES, m/z): [M + H]⁺ = 457. ¹H NMR (400 MHz, DMSO-d₆): δ 1.49 (s, 3H), 3.16 (s, 3H), 3.22 (s, 3H), 3.49-3.60 (m, 2H), 4.29 (s, 3H), 6.71 (d, J = 7.4 Hz, 1H), 6.93- 7.06 (m, 1H), 7.47 (dd, J = 5.2, 1.6 Hz, 1H), 7.71 (dd, J = 8.2, 1.0 Hz, 1H), 7.97 (d, J = 1.7 Hz, 1H), 8.03 (d, J = 0.8 Hz, 1H), 8.10 (s, 1H), 8.49 (d, J = 5.2 Hz, 1H), 8.79 (d, J = 0.8 Hz, 1H), 10.03 (s, 1H). |
| I-301 | 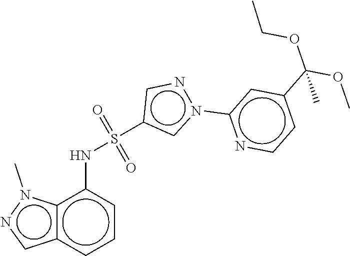 | LC-MS (ES, m/z): [M + H]⁺ = 457. ¹H NMR (400 MHz, DMSO-d₆): δ 1.51 (s, 3H), 3.11 (s, 3H), 3.24 (s, 3H), 3.55 (d, J = 5.2 Hz, 2H), 4.33 (s, 3H), 6.69 (d, J = 7.2 Hz, 1H), 6.95-7.06 (m, 1H), 7.50 (dd, J = 5.2, 1.6 Hz, 1H), 7.74 (dd, J = 8.1, 1.0 Hz, 1H), 7.97 (d, J = 0.8 Hz, 1H), 8.06 (d, J = 0.8 Hz, 1H), 8.12 (s, 1H), 8.49 (d, J = 5.2 Hz, 1H), 8.81 (d, J = 0.8 Hz, 1H), 10.03 (s, 1H). |
| I-302 | 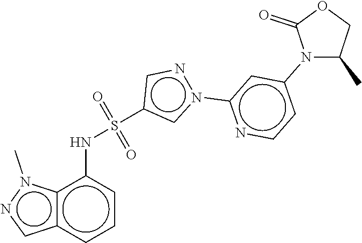 | LC-MS (ES, m/z): [M + H]⁺ = 454. ¹H NMR (400 MHz, DMSO-d₆): δ 1.35 (d, J = 6.2 Hz, 3H), 4.17 (dd, J = 8.4, 3.6 Hz, 1H), 4.28 (s, 3H), 4.60 (t, J = 8.4 Hz, 1H), 4.76-4.88 (m, 1H), 6.70 (d, J = 7.2 Hz, 1H), 6.98 (t, J = 7.6 Hz, 1H), 7.53 (dd, J = 5.6, 2.0 Hz, 1H), 7.70 (d, J = 8.0 Hz, 1H), 8.03 (s, 1H), 8.09 (s, 1H), 8.36 (d, J = 2.0 Hz, 1H), 8.45 (d, J = 5.6 Hz, 1H), 8.78 (d, J = 0.8 Hz, 1H), 10.03 (s, 1H). |
| I-303 | 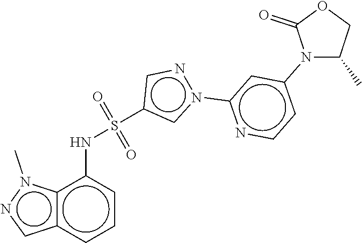 | LC-MS (ES, m/z): [M + H]⁺ = 454. ¹H NMR (400 MHz, DMSO-d₆): δ 1.35 (d, J = 6.0 Hz, 3H), 4.17 (dd, J = 8.4, 3.6 Hz, 1H), 4.28 (s, 3H), 4.60 (t, J = 8.4 Hz, 1H), 4.80-4.85 (m, 1H), 6.70 (d, J = 7.2 Hz, 1H), 6.98 (t, J = 7.6 Hz, 1H), 7.53 (dd, J = 5.6, 2.4 Hz, 1H), 7.70 (d, J = 8.0 Hz, 1H), 8.03 (s, 1H), 8.09 (s, 1H), 8.36 (d, J = 2.0 Hz, 1H), 8.45 (d, J = 5.6 Hz, 1H), 8.78 (s, 1H), 10.03 (s, 1H). |
| I-304 | 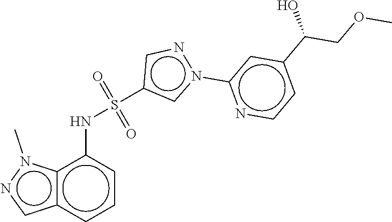 | LC-MS (ES, m/z): [M + H]⁺ = 429. ¹H NMR (400 MHz, DMSO-d₆): δ 3.28 (s, 3H), 3.49-3.54 (m, 2H), 4.29 (s, 3H), 4.87 (t, J = 5.6 Hz, 1H), 5.83 (d, J = 4.8 Hz, 1H), 6.71 (d, J = 7.4 Hz, 1H), 6.98 (t, J = 7.8 Hz, 1H), 7.46 (dd, J = 5.2, 1.4 Hz, 1H), 7.71 (d, J = 8.0 Hz, 1H), 8.02 (d, J = 1.6 Hz, 2H), 8.10 (s, 1H), 8.44 (d, J = 5.2 Hz, 1H), 8.80 (s, 1H), 10.03 (s, 1H). |
| I-305 | 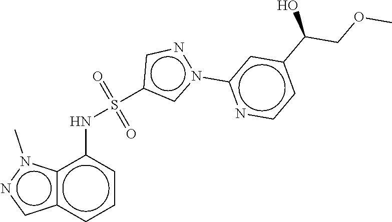 | LC-MS (ES, m/z): [M + H]⁺ = 429. ¹H NMR (400 MHz, DMSO-d₆): δ 3.28 (s, 3H), 3.54-3.49 (m, 2H), 4.29 (s, 3H), 4.87 (t, J = 5.6 Hz, 2H), 6.71 (d, J = 7.4 Hz, 1H), 6.98 (t, J = 7.8 Hz, 1H), 7.46 (dd, J = 5.2, 1.4 Hz, 1H), 7.71 (d, J = 8.0 Hz, 1H), 8.02 (d, J = 1.6 Hz, 2H), 8.10 (s, 1H), 8.44 (d, J = 5.2 Hz, 1H), 8.80 (s, 1H), 10.03 (s, 1H). |
| I-306 | 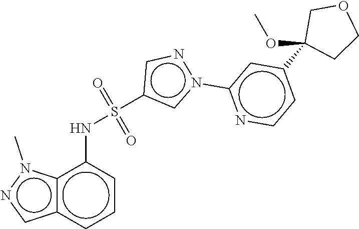 | LC-MS (ES, m/z): [M + H]⁺ = 455. ¹H NMR (400 MHz, DMSO-d₆): δ 2.26-2.38 (m, 1H), 2.46 (d, J = 5.2 Hz, 1H), 3.11 (s, 3H), 3.79 (d, J = 9.6 Hz, 1H), 3.98 (dd, J = 8.4, 5.4 Hz, 2H), 4.08 (d, J = 9.6 Hz, 1H), 4.29 (s, 3H), 6.72 (d, J = 7.2 Hz, 1H), 6.98 (t, J = 7.8 Hz, 1H), 7.51 (dd, J = 5.2, 1.6 Hz, 1H), 7.71 (d, J = 8.0 Hz, 1H), 7.96 (d, J = 1.6 Hz, 1H), 8.04 (s, 1H), 8.09 (s, 1H), 8.54 (d, J = 5.2 Hz, 1H), 8.82 (s, 1H), 10.04 (s, 1H). |
| I-309 | 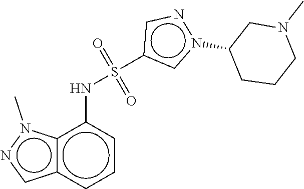 | LC-MS (ES, m/z): [M + 1]⁺ = 375. ¹H NMR (400 MHz, methanol-d₄): δ 1.61-1.87 (m, 3H), 2.03-2.14 (m, 2H), 2.31 (s, 3H), 2.37 (t, J = 10.6 Hz, 1H), 2.75 (d, J = 11.4 Hz, 1H), 3.01 (dd, J = 11.2, 3.8 Hz, 1H), 4.32 (s, 3H), 4.36 (dt, J = 10.2, 4.2 Hz, 1H), 6.65 (d, J = 7.2 Hz, 1H), 6.97 (t, J = 7.8 Hz, 1H), 7.67 (d, J = 10.0 Hz, 2H), 7.94 (s, 1H), 8.00 (s, 1H). |
| I-311 | 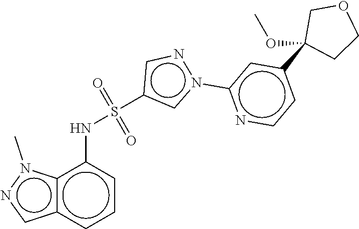 | LC-MS (ES, m/z): [M + H]⁺ = 455. ¹H NMR (400 MHz, DMSO-d₆): δ 2.26-2.38 (m, 1H), 2.46 (d, J = 5.2 Hz, 1H), 3.11 (s, 3H), 3.79 (d, J = 9.6 Hz, 1H), 3.98 (dd, J = 8.4, 5.4 Hz, 2H), 4.08 (d, J = 9.6 Hz, 1H), 4.29 (s, 3H), 6.72 (d, J = 7.2 Hz, 1H), 6.98 (t, J = 7.8 Hz, 1H), 7.51 (dd, J = 5.2, 1.6 Hz, 1H), 7.71 (d, J = 8.0 Hz, 1H), 7.96 (d, J = 1.6 Hz, 1H), 8.04 (s, 1H), 8.09 (s, 1H), 8.54 (d, J = 5.2 Hz, 1H), 8.82 (s, 1H), 10.04 (s, 1H). |
| I-316 | 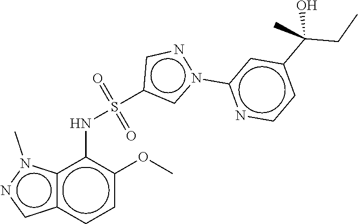 | LC-MS (ES, m/z): [M + 1]⁺ = 457. ¹H NMR (400 MHz, methanol-d₄): δ 0.82 (t, J = 7.6 Hz, 3H), 1.54 (s, 3H), 1.85 (q, J = 7.6 Hz, 2H), 3.39 (s, 3H), 4.34 (s, 2H), 6.84 (d, J = 8.8 Hz, 1H), 7.42 (dd, J =1.6, 5.2 Hz, 1H), 7.66 (d, J = 8.8 Hz, 1H), 7.83 (d, J = 0.8 Hz, 1H), 7.93 (s, 1H), 8.10 (d, J = 1.6 Hz, 1H), 8.38 (d, J = 5.2 Hz, 1H), 8.72 (s, 1H). |
| I-319 | 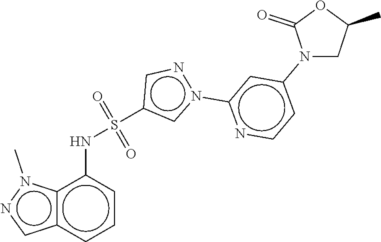 | LC-MS (ES, m/z): [M + H]⁺ = 454. ¹H NMR (400 MHz, DMSO-d₆): δ 1.45 (d, J = 6.2 Hz, 3H), 3.74-3.82 (m, 1H), 4.28 (m, 4H), 4.84-4.97 (m, 1H), 6.71 (d, J = 8 Hz, 1H), 6.98 (t, J = 8 Hz, 1H), 7.45-7.53 (m, 1H), 7.71 (d, J = 8.0 Hz, 1H), 8.03 (s, 1H), 8.09 (s, 1H), 8.29 (d, J = 2 Hz, 1H), 8.43 (d, J = 4 Hz, 1H), 8.79 (s, 1H), 10.03 (s, 1H). |
| I-320 | 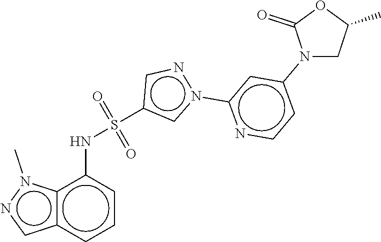 | LC-MS (ES, m/z): [M + H]⁺ = 454. ¹H NMR (400 MHz, DMSO-d₆): δ 1.45 (d, J = 6.2 Hz, 3H), 3.74-3.82 (m, 1H), 4.28 (m, 4H), 4.84-4.97 (m, 1H), 6.71 (d, J = 8 Hz, 1H), 6.98 (t, J = 8 Hz, 1H), 7.45-7.53 (m, 1H), 7.71 (d, J = 8.0 Hz, 1H), 8.03 (s, 1H), 8.09 (s, 1H), 8.29 (d, J = 2 Hz, 1H), 8.43 (d, J = 4 Hz, 1H), 8.79 (s, 1H), 10.03 (s, 1H). |
| I-321 | 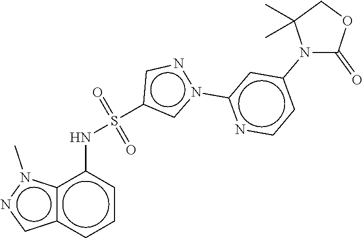 | LC-MS (ES, m/z): [M + H]⁺ = 468. ¹H NMR (400 MHz, DMSO-d₆): δ 1.52 (s, 6H), 4.24 (s, 2H), 4.30 (s, 3H), 6.73 (d, J = 7.4 Hz, 1H), 6.96 (t, J = 7.8 Hz, 1H), 7.46-7.51 (m, 1H), 7.62 (s, 1H), 8.02 (m, 1H), 8.05 (s, 1H), 8.10 (s, 1H), 8.48 (d, J = 5.8 Hz, 1H), 8.77 (s, 1H), 10.00 (s, 1H). |
| I-322 | 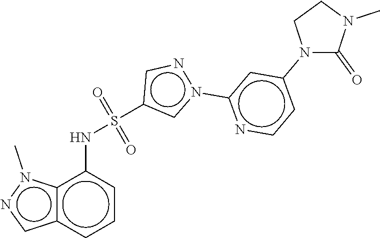 | LC-MS (ES, m/z): [M + H]⁺ = 453. ¹H NMR (400 MHz, DMSO-d₆): δ 2.81 (s, 3H), 3.52 (t, J = 8.0 Hz, 2H), 3.89 (t, J =7.2 Hz, 2H), 4.31 (s, 3H), 6.74 (d, J = 7.2 Hz, 1H), 6.94 (t, J = 7.6 Hz, 1H), 7.47-7.55 (m, 2H), 7.94 (s, 1H), 8.16(s, 1H), 8.24 (d, J = 2.0 Hz, 1H), 8.30 (d, J = 6.0 Hz, 1H), 8.71 (s, 1H), 10.50(s, 1H). |
| I-325 | 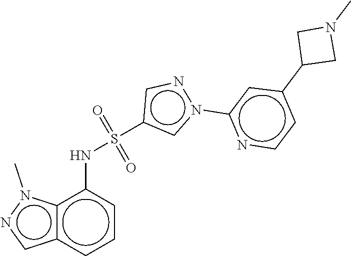 | LC-MS (ES, m/z): [M + 1]⁺ = 424. ¹H NMR (400 MHz, methanol-d₄): 82.43 (s, 3H), 3.32-3.45 (m, 2H), 3.80-3.91 (m, 3H), 4.37 (s, 3H), 6.75 (dd, J = 7.4, 0.8 Hz, 1H), 6.95 (t, J = 7.8 Hz, 1H), 7.35 (dd, J = 5.1, 1.6 Hz, 1H), 7.67 (dd, J = 8.1, 0.8 Hz, 1H), 7.85 (s, 1H), 7.96 (d, J = 1.6 Hz, 1H), 8.01 (s, 1H), 8.38 (d, J = 5.2 Hz, 1H), 8.77 (s, 1H). |
| I-326 | 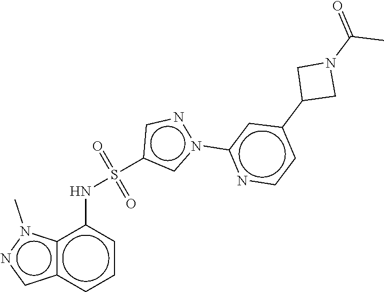 | LC-MS (ES, m/z): [M + 1]⁺ = 452. ¹H NMR (400 MHz, DMSO-d₆): δ 1.81 (s, 3H), 3.87 (dd, J = 9.4, 6.2 Hz, 1H), 4.00 (tt, J = 8.6, 5.8 Hz, 1H), 4.17-4.30 (m, 2H), 4.32 (s, 3H), 4.53 (t, J = 8.6 Hz, 1H), 6.75 (d, J = 7.2 Hz, 1H), 6.89 (t, J = 7.8 Hz, 1H), 7.44 (s, 1H), 7.47 (dd, J = 5.2, 1.6 Hz, 1H), 7.91- 8.00 (m, 3H), 8.47 (d, J = 5.2 Hz, 1H), 8.73 (s, 1H). |
| I-328 | 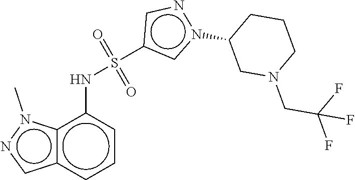 | LC-MS (ES, m/z): [M + 1]⁺ = 443.10. ¹H NMR (400 MHz, methanol-d₄): δ 1.65-1.75 (m, 2H), 1.91-1.79 (m, 1H), 2.00 (dt, J = 9.4, 4.4 Hz, 1H), 2.52-2.58 (m, 1H), 2.78-2.87 (m, 2H), 3.09-3.16 (m, 3H), 4.32 (s, 3H), 4.39 (tt, J = 8.6, 4.0 Hz, 1H), 6.64 (d, J = 7.4 Hz, 1H), 6.96 (t, J = 7.8 Hz, 1H), 7.66-7.70 (m, 2H), 7.98 (s, 1H), 8.01 (s, 1H). |
| I-329 | 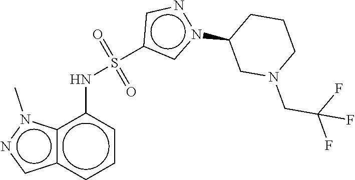 | LC-MS (ES, m/z): [M + 1]⁺ = 443. ¹H NMR (400 MHz, methanol-d₄): δ 1.66-1.72 (m, 2H), 1.78-1.91 (m, 1H), 1.95-2.05 (m, 1H), 2.51-2.57 (m, 1H), 2.75-2.90 (m, 2H), 3.06- 3.18 (m, 3H), 4.33 (s, 3H), 4.35- 4.40 (m, 1H), 6.67 (dd, J = 7.4, 1.2 Hz, 1H), 6.95 (t, J = 7.8 Hz, 1H), 7.59-7.68 (m, 2H), 7.98 (d, J = 4.4 Hz, 2H). |
| I-332 | 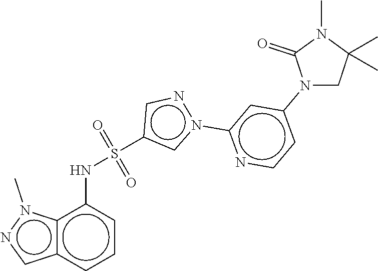 | LC-MS (ES, m/z): [M + H]⁺ = 481. ¹H NMR (400 MHz, DMSO-d₆): δ 1.31 (s, 6H), 2.73 (s, 3H), 3.71 (s, 2H), 4.29 (s, 3H), 6.71 (d, J = 7.4 Hz, 1H), 6.95 (m, 1H), 7.51 (m, J = 5.8, 2.2 Hz, 1H), 7.64 (m, 1H), 7.98 (s, 1H), 8.05 (s, 1H), 8.22 (s, 1H), 8.30 (d, J = 5.8 Hz, 1H), 8.73 (s, 1H), 10.01 (s, 1H) |
| I-333 | 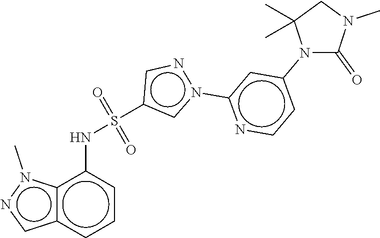 | LC-MS (ES, m/z): [M + H]⁺ = 481. ¹H NMR (400 MHz, DMSO-d₆): δ 1.52 (s, 6H), 2.82 (s, 3H), 3.22-3.46 (s, 2H), 4.29 (s, 3H), 6.71 (d, J = 7.4 Hz, 1H), 6.98 (t, J = 7.8 Hz, 1H), 7.48 (m, J = 5.8, 2.2 Hz, 1H), 7.67 (s, 1H), 7.99 (s, 1H), 8.08 (s, 1H), 8.18 (d, J = 2.2 Hz, 1H), 8.34 (d, J = 5.8 Hz, 1H), 8.75 (s, 1H), 10.01 (s, 1H). |
| I-334 | 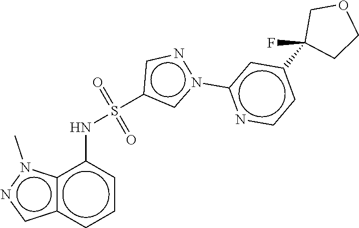 | LC-MS (ES, m/z): [M + H]⁺ = 443. ¹H NMR (400 MHz, methanol-d₄): δ 2.57-2.59 (m, 2H), 4.05 (d, J = 10.8 Hz, 1H), 4.11-4.26 (m, 3H), 4.39 (s, 3H), 6.85-6.94 (m, 2H), 7.44-7.48 (m, 2H), 7.86 (s, 1H), 7.93 (s, 1H), 8.07 (s, 1H), 8.46 (d, J = 5.2 Hz, 1H), 8.77 (s, 1H). |
| I-335 | 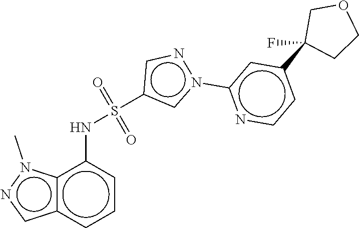 | LC-MS (ES, m/z): [M + H]⁺ = 443. ¹H NMR (400 MHz, methanol-d₄): δ 2.44-2.61 (m, 2H), 4.05 (d, J = 10.8 Hz, 1H), 4.11-4.26 (m, 3H), 4.39 (s, 3H), 6.84-6.96 (m, 2H), 7.41-7.48 (m, 2H), 7.86 (s, 1H), 7.92 (s, 1H), 8.06 (d, J = 1.6 Hz, 1H), 8.46 (d, J = 5.2 Hz, 1H), 8.77 (s, 1H). |
| I-341 | 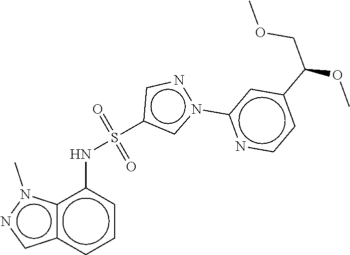 | LC-MS (ES, m/z): [M + H]⁺ = 443. ¹H NMR (400 MHz, methanol-d₄): δ 3.37 (d, J = 7.8 Hz, 6H), 3.56 (dd, J = 10.6, 4.6 Hz, 1H), 3.63 (dd, J = 10.6, 6.4 Hz, 1H), 4.37 (s, 3H), 4.54 (dd, J = 6.4, 4.4 Hz, 1H), 6.73 (dd, J = 7.4, 1.0 Hz, 1H), 6.97 (t, J = 7.8 Hz, 1H), 7.38 (dd, J = 5.0, 1.4 Hz, 1H), 7.71 (dd, J = 8.0, 1.0 Hz, 1H), 7.86 (s, 1H), 8.02 (d, J = 4.0 Hz, 2H), 8.43 (d, J = 5.2 Hz, 1H), 8.79 (s, 1H). |
| I-342 | 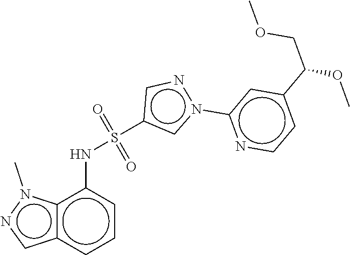 | LC-MS (ES, m/z): [M + H]⁺ = 443. ¹H NMR (400 MHz, methanol-d₄): δ 3.37 (d, J = 7.8 Hz, 6H), 3.56 (dd, J = 10.6, 4.6 Hz, 1H), 3.63 (dd, J = 10.6, 6.4 Hz, 1H), 4.37 (s, 3H), 4.54 (dd, J = 6.4, 4.4 Hz, 1H), 6.73 (dd, J = 7.4, 1.0 Hz, 1H), 6.97 (t, J = 7.8 Hz, 1H), 7.38 (dd, J = 5.0, 1.4 Hz, 1H), 7.71 (dd, J = 8.0, 1.0 Hz, 1H), 7.86 (s, 1H), 8.02 (d, J = 4.0 Hz, 2H), 8.43 (d, J = 5.2 Hz, 1H), 8.79 (s, 1H). |
| I-344 | 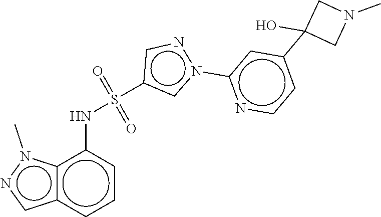 | LC-MS (ES, m/z): [M + 1]⁺ = 440. ¹H NMR (400 MHz, CD₃OD): δ 2.57(s, 3H), 3.58 (d, J = 9.2 Hz, 2H), 3.87 (d, J = 8.8 Hz, 2H), 4.37 (s, 3H), 6.73 (t, J = 8.4 Hz, 1H), 6.96 (t, J =7.6 Hz, 1H), 7.65-7.71 (m, 2H), 7.86 (s, 1H), 8.03 (s, 1H), 8.28 (s, 1H), 8.46 (d, J = 5.2 Hz, 1H), 8.80 (s, 1H). |
| I-345 | 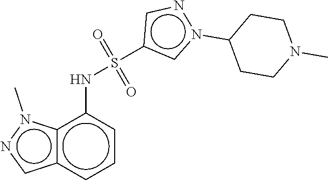 | LC-MS (ES, m/z): [M + 1]⁺ = 375. ¹H NMR (400 MHz, DMSO-d₆): δ 1.61-1.98 (m, 4H), 1.95-2.08 (m, 2H), 2.19 (s, 3H), 2.82 (d, J = 10.6 Hz, 2H), 4.18-4.22 (m, 1H), 4.23 (s, 3H), 6.61 (d, J = 7.2 Hz, 1H), 6.84- 7.06 (m, 1H), 7.62 (d, J = 8.0 Hz, 1H), 7.67 (s, 1H), 8.04 (d, J = 4.4 Hz, 1H), 8.09 (s, 1H), 9.72 (s, 1H). |
| I-346 | 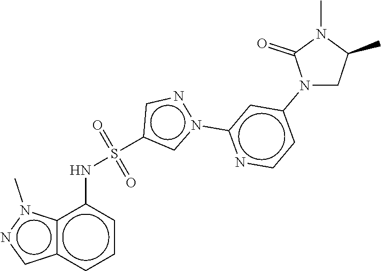 | LC-MS (ES, m/z): [M + H]⁺ = 467. ¹H NMR (400 MHz, DMSO-d₆): δ 1.28 (d, J = 6.2 Hz, 3H), 2.78 (s, 3H), 3.44 (m, J = 9.6, 6.8 Hz, 1H), 3.77 (m, J = 14.8, 6.4 Hz, 1H), 4.08 (t, J = 9.2 Hz, 1H), 4.29 (s, 3H), 6.71 (m, J = 7.8, 1.0 Hz, 1H), 6.96 (t, J = 7.8 Hz, 1H), 7.50 (m, J = 5.8, 2.2 Hz, 1H), 7.62 (s, 1H), 7.99 (s, 1H), 8.06 (s, 1H), 8.25 (d, J = 2.2 Hz, 1H), 8.31 (d, J = 5.8 Hz, 1H), 8.74 (s, 1H), 10.07 (s, 1H). |
| I-348 | 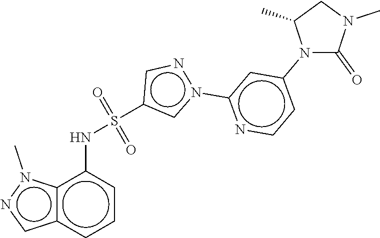 | LC-MS (ES, m/z): [M + H]⁺ = 467. ¹H NMR (400 MHz, DMSO-d₆): δ 1.30 (d, J = 6.0 Hz, 3H), 2.82 (s, 3H), 3.16 (m, J = 9.0, 2.8 Hz, 1H), 3.66 (t, J = 8.8 Hz, 1H), 4.28 (s, 3H), 4.56 (m, J = 8.8, 6.2, 2.8 Hz, 1H), 6.70 (d, J = 7.4 Hz, 1H), 6.98 (t, J = 8.0 Hz, 1H), 7.52 (m, J = 5.8, 2.2 Hz, 1H), 7.70 (d, J = 8.0 Hz, 1H), 8.00 (s, 1H), 8.09 (s, 1H), 8.32 (d, J = 5.8 Hz, 1H), 8.38 (d, J = 2.2 Hz, 1H), 8.75 (s, 1H), 10.02 (s, 1H). |
| I-349 | 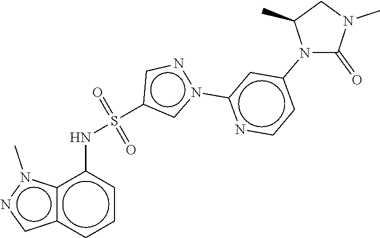 | LC-MS (ES, m/z): [M + H]⁺ = 467. ¹H NMR (400 MHz, DMSO-d₆): δ 1.30 (d, J = 6.0 Hz, 3H), 2.82 (s, 3H), 3.16 (m, J = 9.0, 2.8 Hz, 1H), 3.66 (t, J = 8.8 Hz, 1H), 4.28 (s, 3H), 4.56 (m, J = 8.8, 6.2, 2.8 Hz, 1H), 6.70 (d, J = 7.4 Hz, 1H), 6.98 (t, J = 8.0 Hz, 1H), 7.52 (m, J = 5.8, 2.2 Hz, 1H), 7.70 (d, J = 8.0 Hz, 1H), 8.00 (s, 1H), 8.09 (s, 1H), 8.32 (d, J = 5.8 Hz, 1H), 8.38 (d, J = 2.2 Hz, 1H), 8.75 (s, 1H), 10.02 (s, 1H). |
| I-350 | 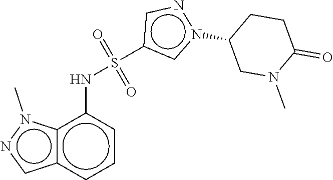 | LC-MS (ES, m/z): [M + 1]⁺ = 389. ¹H NMR (400 MHz, DMSO-d₆): δ 2.15-2.22 (m, 2H), 2.33-2.50 (m, 2H), 2.82 (s, 3H), 3.66 (d, J = 6.4 Hz, 2H), 4.24 (s, 3H), 4.78-4.82 (m, 1H), 6.58 (d, J = 7.2 Hz, 1H), 6.97 (t, J = 7.6 Hz, 1H), 7.68 (d, J = 8.0 Hz, 1H), 7.74 (s, 1H), 8.06 (s, 1H), 8.18 (s, 1H), 9.78 (s, 1H). |
| I-351 | 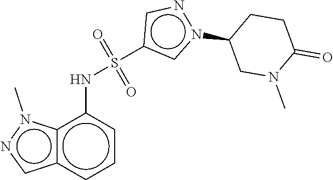 | LC-MS (ES, m/z): [M + 1]⁺ = 389. ¹H NMR (400 MHz, DMSO-d₆): δ 2.15-2.22 (m, 2H), 2.33-2.50 (m, 2H), 2.82 (s, 3H), 3.66 (d, J = 6.4 Hz, 2H), 4.24 (s, 3H), 4.78-4.82 (m, 1H), 6.58 (d, J = 7.2 Hz, 1H), 6.97 (t, J = 7.6 Hz, 1H), 7.68 (d, J = 8.0 Hz, 1H), 7.74 (s, 1H), 8.06 (s, 1H), 8.18 (s, 1H), 9.78 (s, 1H). |
| I-358 | 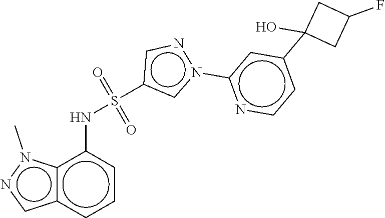 | LC-MS (ES, m/z): [M + H]⁺ = 443. ¹H NMR (400 MHz, DMSO-d₆): δ 2.52-2.83 (m, 4H), 4.29 (s, 3H), 5.43 (dt, J = 6.4, 56.4 Hz, 1H), 6.14 (s, 1H), 6.71 (dd, J = 1.2, 7.2 Hz, 1H), 6.97 (t, J = 7.6 Hz, 1H), 7.56 (dd, J = 1.6, 5.2 Hz, 1H), 7.68 (d, J = 8.0 Hz, 1H), 8.02 (s, 1H), 8.05 (s, 1H), 8.08 (s, 1H), 8.50 (d, J = 5.2 Hz, 1H), 8.80 (s, 1H), 10.03 (s, 1H). |
| I-359 | 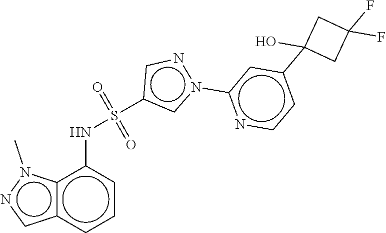 | LC-MS (ES, m/z): [M + H]⁺ = 461. ¹H NMR (400 MHz, DMSO-d₆): δ 2.95-3.02 (m, 2H), 3.15-3.30 (m, 2H), 4.28 (s, 3H), 6.57 (s, 1H), 6.71 (d, J = 7.2 Hz, 1H), 6.97 (t, J = 7.6, 1H), 7.56 (d, J = 5.2 Hz, 1H), 7.69 (t, J = 8.0 Hz, 1H), 8.03 (s, 1H), 8.05 (s, 1H), 8.08 (s, 1H), 8.52 (d, J = 5.2 Hz, 1H), 8.81 (d, J = 2.0 Hz, 1H), 10.03 (s, 1H). |
| I-367 | 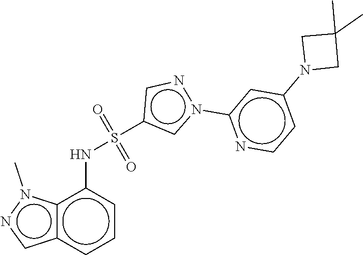 | LC-MS (ES, m/z): [M + H]⁺ = 438. ¹H NMR (400 MHz, DMSO-d₆): δ 1.30 (s, 6H), 3.71 (s, 4H), 4.29 (s, 3H), 6.36 (dd, J = 5.8, 4 Hz, 1H), 6.71 (d, J = 8.0 Hz, 1H), 6.80 (d, J = 2.0 Hz, 1H), 6.93 (t, J = 8.0 Hz, 1H), 7.57 (s, 1H), 7.90 (s, 1H), 8.00 (d, J = 4.0 Hz, 1H), 8.02 (s, 1H), 8.66 (s, 1H), 9.96 (s, 1H). |
| I-369 | 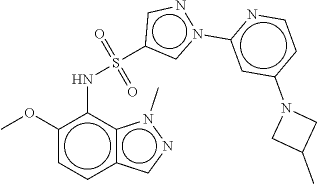 | LC-MS (ES, m/z): [M + 1]⁺ = 454. ¹H NMR (400 MHz, chloroform-d): δ 1.33 (d, J = 6.8 Hz, 3H), 2.87-2.95 (m, 1H), 3.39 (s, 3H), 3.59 (dd, J = 7.8, 5.4 Hz, 2H), 4.15 (t, J = 7.8 Hz, 2H), 4.40 (s, 3H), 6.18 (dd, J = 5.8, 2.2 Hz, 1H), 6.48 (s, 1H), 6.66 (d, J = 8.8 Hz, 1H), 6.80 (d, J = 2.2 Hz, 1H), 7.55-7.65 (m, 2H), 7.92 (s, 1H), 7.97 (d, J = 5.8 Hz, 1H), 8.71 (d, J = 0.8 Hz, 1H). |
| I-372 | 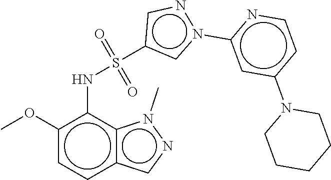 | LC-MS (ES, m/z): [M + 1]⁺ = 468. ¹H NMR (400 MHz, chloroform-d): δ 1.68 (s, 6H), 3.40-3.43 (m, 7H), 4.40 (s, 3H), 6.48 (s, 1H), 6.60 (dd, J = 6.2, 2.6 Hz, 1H), 6.66 (d, J = 8.8 Hz, 1H), 7.28 (d, J = 2.6 Hz, 1H), 7.65-7.56 (m, 2H), 7.92 (s, 1H), 8.01 (d, J = 6.0 Hz, 1H), 8.72 (s, 1H). |
| I-384 | 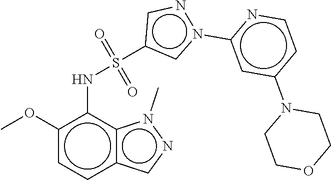 | LC-MS (ES, m/z): [M + 1]⁺ = 449. ¹H NMR (400 MHz, DMSO-d₆): δ 3.32 (s, 3H), 3.40 (d, J = 5.0 Hz, 4H), 3.73 (t, J = 4.9 Hz, 4H), 4.24 (s, 3H), 6.87 (d, J = 8.9 Hz, 1H), 6.91 (dd, J = 6.1, 2.4 Hz, 1H), 7.32 (d, J = 2.4 Hz, 1H), 7.67 (d, J = 8.8 Hz, 1H), 7.91 (s, 1H), 7.98 (s, 1H), 8.11 (d, J = 6.0 Hz, 1H), 8.64 (s, 1H), 9.71 (s, 1H). |
| I-385 | 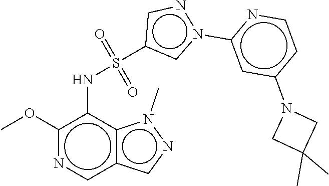 | LC-MS (ES, m/z): [M + H]⁺ 469. ¹H NMR (400 MHz, DMSO-d₆): δ 1.31 (s, 6H), 3.42 (s, 3H), 3.72 (s, 4H), 4.21 (s, 3H), 6.37 (dd, J = 2.4, 5.6 Hz, 1H), 6.80 (d, J = 2.0 Hz, 1H), 7.91 (s, 1H), 8.02 (d, J = 5.6 Hz, 1H), 8.21 (s, 1H), 8.64 (s, 2H), 9.84 (s, 1H). |
| I-392 | 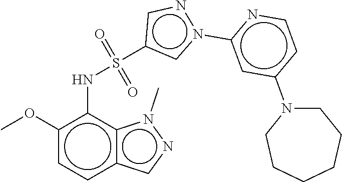 | LC-MS (ES, m/z): [M + 1]⁺ = 482. ¹H NMR (400 MHz, DMSO-d₆): δ 1.49 (s, 4H), 1.74 (s, 4H), 3.34 (s, 3H), 3.55 (t, J = 6.0 Hz, 4H), 4.24 (s, 3H), 6.66-6.72 (m, 1H), 6.87 (d, J = 8.8 Hz, 1H), 7.11 (d, J = 2.4 Hz, 1H), 7.66 (d, J = 8.8 Hz, 1H), 7.90 (s, 1H), 7.97 (s, 1H), 8.01 (d, J = 6.0 Hz, 1H), 8.62 (s, 1H), 9.70 (s, 1H). |
| I-397 | 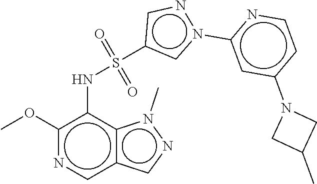 | LC-MS (ES, m/z): [M + H]⁺ 455. ¹H NMR (400 MHz, DMSO-d₆): δ 1.26 (d, J = 6.9 Hz, 3H), 2.85-2.89 (m, 1H), 3.34 (s, 3H), 3.58 (dd, J = 5.2, 8.0 Hz, 2H), 4.13 (t, J = 8.0 Hz, 2H), 4.21 (s, 3H), 6.36 (dd, J = 2.0, 5.6 Hz, 1H), 6.80 (d, J = 2.4 Hz, 1H), 7.91 (s, 1H), 8.02 (d, J = 5.6 Hz, 1H), 8.22 (s, 1H), 8.64 (s, 1H), 8.65 (s, 1H), 9.86 (s, 1H). |
| I-402 | 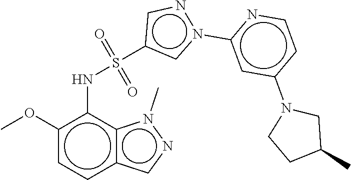 | LC-MS (ES, m/z): [M + H]⁺ = 468. ¹H NMR (400 MHz, chloroform-d): δ 1.15 (d, J = 6.6 Hz, 3H), 1.68 (d, J = 12.4 Hz, 1H), 2.18 (dd, J = 12.8, 6.8 Hz, 1H), 2.37-2.50 (m, J = 7.2 Hz, 1H), 2.95 (dd, J = 9.6, 7.8 Hz, 1H), 3.40 (s, 4H), 3.44-3.59 (m, 2H), 4.40 (s, 3H), 6.33 (dd, J = 5.8, 2.4 Hz, 1H), 6.45 (s, 1H), 6.66 (d, J = 8.8 Hz, 1H), 6.97 (d, J = 2.4 Hz, 1H), 7.56-7.65 (m, 2H), 7.92 (s, |
| | | 1H), 7.98 (d, J = 5.8 Hz, 1H), 8.73 |
| | | (s, 1H). |
| I-403 | 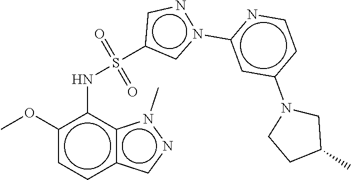 | LC-MS (ES, m/z): [M + H]⁺ = 468. ¹H NMR (400 MHz, DMSO-d₆): δ 1.07 (d, J = 6.6 Hz, 3H), 1.54-1.65 (m, 1H), 2.03-2.13 (m, 1H), 2.34- 2.42 (m, 1H), 2.88 (dd, J = 10.0, 7.6 Hz, 1H), 3.29 (s, 3H), 3.30-3.35 (m, 1H), 3.40-3.46 (m, 1H), 3.51 (dd, J = 9.6, 7.2 Hz, 1H), 4.21 (s, 3H), 6.49 (dd, J = 6.0, 2.4 Hz, 1H), 6.85 (d, J = 8.8 Hz, 1H), 6.92 (d, J = 2.4 Hz, 1H), 7.66 (d, J = 8.8 Hz, 1H), |
| | | 7.86 (s, 1H), 7.97(s, 1H), 7.99-8.01 |
| | | (d, J = 1.2 Hz, 1H), 8.61 (s, 1H). |
| I-404 | 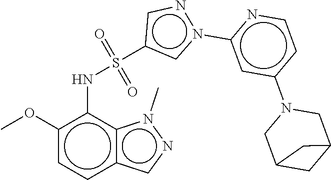 | LC-MS (ES, m/z): [M + H]⁺ = 480. ¹H NMR (400 MHz, chloroform-d): δ 1.45 (d, J = 7.8 Hz, 2H), 2.27 (dt, J = 8.7, 6.2 Hz, 2H), 2.66 (t, J = 6.4 Hz, 2H), 3.62 (s, 4H), 3.41 (s, 3H), 4.41 (s, 3H), 6.48 (s, 1H), 6.52 (dd, J = 6.0, 2.4 Hz, 1H), 6.66 (d, J = 8.8 Hz, 1H), 7.17 (d, J = 2.4 Hz, 1H), 7.56-7.66 (m, 2H), 7.92 (s, 1H), 8.05 (d, J = 6.0 Hz, 1H), 8.80 (s, 1H). |
| I-405 | 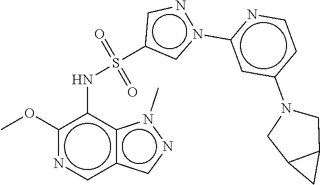 | LC-MS (ES, m/z): [M + 1]⁺ = 467. ¹H NMR (400 MHz, DMSO-d₆): δ 0.21 (q, J = 4.4 Hz, 1H), 0.76-0.85 (m, 1H), 1.72-1.80 (m, 2H), 3.39- 3.46 (m, 2H), 3.43 (s, 3H), 3.57 (d, J = 10.0 Hz, 2H), 4.22 (s, 3H), 6.53 (dd, J = 2.4, 6.0 Hz, 1H), 6.96 (d, J = 2.4 Hz, 1H), 7.92 (s, 1H), 8.02 (d, J = 6.0 Hz, 1H), 8.22 (s, 1H), 8.64 (s, 1H), 8.66 (s, 1H), 9.84 (s, 1H). |
| I-406 | 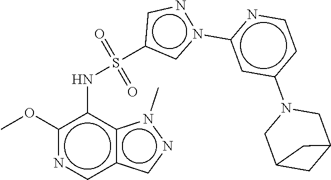 | LC-MS (ES, m/z): [M + 1]⁺ = 481. ¹H NMR (400 MHz, DMSO-d₆): δ 1.41 (dd, J = 2.8, 6.8 Hz, 2H), 2.20 (dd, J = 2.8, 6.4 Hz, 1H), 2.61 (t, J = 6.4 Hz, 2H), 3.43 (s, 3H), 3.58 (s, 4H), 4.22 (s, 3H), 6.68 (dd, J = 2.4, 6.0 Hz, 1H), 7.15 (d, J = 2.4 Hz, 1H), 7.92 (d, J = 0.8 Hz, 1H), 8.08 (d, J = 6.0 Hz, 1H), 8.22 (s, 1H), 8.65 (s, 1H), 8.68 (t, J = 0.4 Hz, 1H), 9.84 (s, 1H). |
| I-407 | 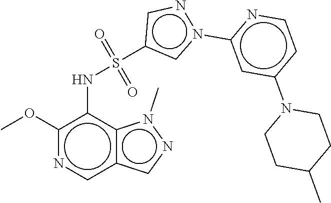 | LC-MS (ES, m/z): [M + H]⁺ = 483. ¹H NMR (400 MHz, DMSO-d₆) 0.93 (d, J = 6.2 Hz, 3H), 1.04-1.25 (m, 2H), 1.47-1.82 (m, 3H), 2.93 (t, J = 12.8 Hz, 2H), 3.43 (s, 3H), 3.98 (d, J = 13.2 Hz, 2H), 4.21 (s, 3H), 6.88 (dd, J = 2.4, 6.0 Hz, 1H), 7.28 (d, J = 2.4 Hz, 1H), 7.92 (d, J = 0.8 Hz, 1H), 8.04 (d, J = 6.4 Hz, 1H), 8.22 (s, 1H), 8.64 (s, 1H), 8.65 (d, J = 1.2 Hz, 1H), 9.87 (s, 1H). |
| I-408 | 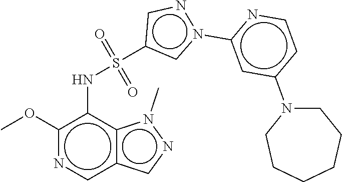 | LC-MS (ES, m/z): [M + H]⁺ = 483. ¹H NMR (400 MHz, DMSO-d₆): δ 1.40-1.56 (m, 4H), 1.65-1.88 (m, 4H), 3.43 (s, 3H), 3.52-3.56 (m, 4H), 4.21 (s, 3H), 6.70 (dd, J = 2.4, 6.0 Hz, 1H), 7.11 (d, J = 2.4 Hz, 1H), 7.91(s, 1H), 8.05 (d, J = 6.0 Hz, 1H), 8.22 (d, J = 1.2 Hz, 1H), 8.64(s, 1H), 8.65 (d, J = 1.2 Hz, 1H), 9.77 (s, 1H). |
| I-414 | 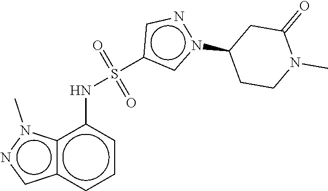 | LC-MS (ES, m/z): [M + 1]⁺ = 389. ¹H NMR (400 MHz, DMSO-d₆): δ 2.05-2.26 (m, 2H), 2.68-2.77 (m, 2H), 2.82 (s, 3H), 3.18 (m, 1H), 3.35 (s, 1H), 4.23 (s, 3H), 4.79 (s, 1H), 6.61 (dd, J = 1.2, 7.2 Hz, 1H), 6.99 (t, J = 7.6 Hz, 1H), 7.70 (dd, J = 1.2, 8.0 Hz, 1H), 7.72 (s, 1H), 8.07 (s, 1H), 8.16 (s, 1H), 9.78 (s, 1H). |
| I-415 | 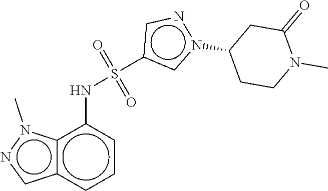 | LC-MS (ES, m/z): [M + 1]⁺ = 389. ¹H NMR (400 MHz, DMSO-d₆): δ 2.05-2.26 (m, 2H), 2.68-2.77 (m, 2H), 2.82 (s, 3H), 3.18 (m, 1H), 3.35 (s, 1H), 4.23 (s, 3H), 4.79 (s, 1H), 6.61 (dd, J = 1.2, 7.2 Hz, 1H), 6.99 (t, J = 7.6 Hz, 1H), 7.70 (dd, J = 1.2, 8.0 Hz, 1H), 7.72 (s, 1H), 8.07 (s, 1H), 8.16 (s, 1H), 9.78 (s, 1H). |
| I-416 | 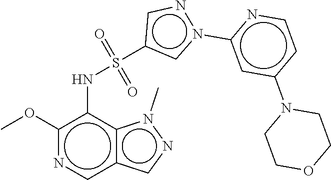 | LC-MS (ES, m/z): [M + H]⁺ = 471. ¹H NMR (400 MHz, DMSO-d₆): δ 3.32 (s, 3H), 3.41 (t, J = 5.2 Hz, 4H), 3.70-3.77 (t, J = 5.2 Hz, 4H), 4.21 (s, 3H), 6.92 (dd, J = 2.4, 6.2 Hz, 1H), 7.32 (d, J = 2.4 Hz, 1H), 7.93 (s, 1H), 8.12 (d, J = 6.0 Hz, 1H), 8.22 (s, 1H), 8.65(s, 1H), 8.67 (s, 1H), 9.88 (s, 1H). |
| I-417 | 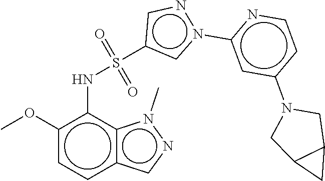 | LC-MS (ES, m/z): [M + 1]⁺ = 466. ¹H NMR (400 MHz, chloroform-d): δ 0.26-0.27 (m, 1H), 0.82-0.87 (m, 1H), 1.69-1.76 (m, 2H), 3.40-3.46 (m, 5H), 3.57 (d, J = 9.6 Hz, 2H), 4.40 (s, 3H), 6.33 (d, J = 5.6 Hz, 1H), 6.43 (s, 1H), 6.66 (d, J = 8.8 Hz, 1H), 6.97 (s, 1H), 7.55-7.65 (m, 2H), 7.92 (s, 1H), 7.97 (d, J = 5.8 Hz, 1H), 8.71 (s, 1H). |
| I-418 | 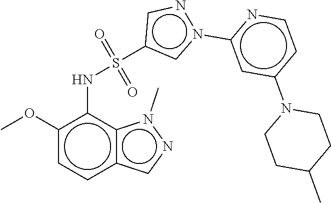 | LC-MS (ES, m/z): [M + 1]⁺ = 482. ¹H NMR (400 MHz, DMSO-d₆): δ 0.93 (d, J = 6.2 Hz, 3H), 1.05-1.20 (m, 2H), 1.65-1.74 (m, 3H), 2.95 (td, J = 12.8, 2.6 Hz, 2H), 3.33 (s, 3H), 3.97 (d, J = 13.4 Hz, 2H), 4.24 (s, 3H), 6.87 (dd, J = 8.6, 5.0 Hz, 2H), 7.27 (d, J = 2.4 Hz, 1H), 7.69 (d, J = 8.8 Hz, 1H), 7.90 (s, 1H), 7.99 (s, 1H), 8.04 (d, J = 6.0 Hz, 1H), 8.64 (s, 1H). |
| I-419 | 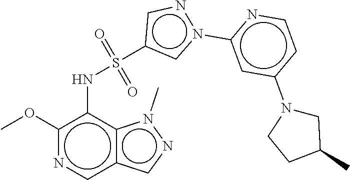 | LC-MS (ES, m/z): [M + H]⁺ = 469. ¹H NMR (400 MHz, CDCl₃): δ 1.09 (d, J = 6.4 Hz, 3H), 1.56-1.68 (m, 1H), 2.01-2.21 (m, 1H), 2.39-2.48 (m, 1H), 2.92 (t, J = 8.8 Hz, 1H), 3.36-3.70 (m, 3H), 3.50 (s, 3H), 4.36 (s, 3H), 6.32 (dd, J = 2.0, 6.0 Hz, 1H), 6.43 (s, 1H), 6.95 (d, J = 4.0 Hz, 1H), 7.27 (s, 1H), 7.68 (s, 1H), 7.97 (d, J = 6.0 Hz, 1H), 8.03 (s, 1H), 8.53 (s, 1H), 8.75 (s, 1H). |
| I-420 | 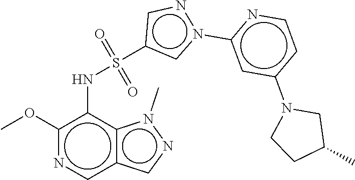 | LC-MS (ES, m/z): [M + H]⁺ 469. ¹H NMR (400 MHz, chloroform-d): δ 1.16 (d, J = 6.6 Hz, 3H), 1.61-1.75 (m, 1H), 2.13-2.25 (m, 1H), 2.37- 2.51 (m, 1H), 2.92-3.01 (m, 1H), 3.39 (q, J = 8.6 Hz, 1H), 3.45-3.60 (m, 2H), 3.55 (s, 3H), 4.36 (s, 3H), 6.31-6.38 (m, 2H), 6.99 (d, J = 2.2 Hz, 1H), 7.68 (s, 1H), 7.97 (d, J = 5.6 Hz, 1H), 8.04 (s, 1H), 8.53 (s, 1H), 8.80 (s, 1H). |
| I-422 | 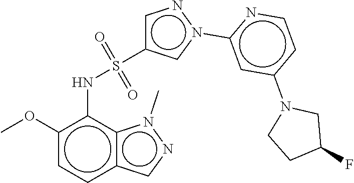 | LC-MS (ES, m/z): [M + 1]⁺ = 472. ¹H NMR (400 MHz, DMSO-d₆): δ 2.25-2.35 (m, 2H), 3.33 (s, 3H), 3.40-3.50 (m, 1H), 3.63 (s, 1H), 3.68 (s, 2H), 4.25 (s, 3H), 5.56 (s, 1H), 6.59 (dd, J = 5.8, 2.4 Hz, 1H), 6.86 (d, J = 8.8 Hz, 1H), 7.01 (d, J = 2.4 Hz, 1H), 7.64 (d, J = 8.8 Hz, 1H), 7.89 (s, 1H), 7.97 (s, 1H), 8.05 (d, J = 5.8 Hz, 1H), 8.64 (s, 1H). |
| I-423 | 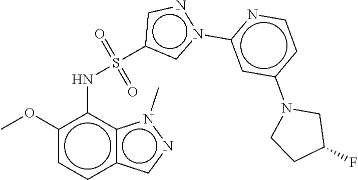 | LC-MS (ES, m/z): [M + H]⁺ = 472. ¹H NMR (400 MHz, DMSO-d₆): δ 2.13-2.40 (m, 2H), 3.32 (s, 3H), 3.46 (t, J = 10.2 Hz, 1H), 3.59-3.70 (m, 3H), 4.24 (s, 3H), 5.37-5.62 (m, 1H), 6.59 (dd, J = 6.0, 2.2 Hz, 1H), 6.87 (d, J = 8.8 Hz, 1H), 7.02 (d, J = 2.2 Hz, 1H), 7.68 (d, J = 8.8 Hz, 1H), 7.91 (s, 1H), 7.99 (s, 1H), 8.05 (d, J = 5.8 Hz, 1H), 8.65 (s, 1H). |
| I-424 | 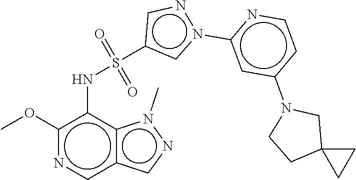 | LC-MS (ES, m/z): [M + H]⁺ 481. ¹H NMR (400 MHz, DMSO-d₆): δ 0.60-0.71 (m, 4H), 1.94 (t, J = 6.8 Hz, 2H), 3.30 (s, 2H), 3.42 (s, 3H), 3.53 (t, J = 6.8 Hz, 2H), 4.22 (s, 3H), 6.50 (d, J = 5.6 Hz, 1H), 6.95 (s, 1H), 7.92 (s, 1H), 8.02 (d, J = 5.6 Hz, 1H), 8.22 (s, 1H), 8.66 (s, 1H), 8.69 (s, 1H), 9.86 (s, 1H). |
| I-425 | 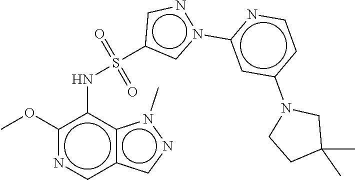 | LC-MS (ES, m/z): [M + H]⁺ = 483. ¹HNMR (400 MHz, DMSO-d₆): δ 1.12 (s, 6H), 1.80 (t, J = 7.2 Hz, 2H), 3.14 (s, 2H), 3.43 (s, 3H), 3.45 (t, J = 7.2 Hz, 2H), 4.22 (s, 3H), 6.50 (d, J = 6.0 Hz, 1H), 6.95 (s, 1H), 7.92 (s, 1H), 8.01 (d, J = 5.8 Hz, 1H), 8.22 (s, 1H), 8.65 (s, 2H), 9.87 (s, 1H). |
| I-426 | 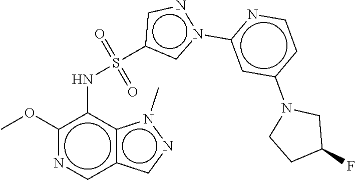 | LC-MS (ES, m/z): [M + H]⁺ = 473. ¹H NMR (400 MHz, DMSO-d₆): δ 2.35-2.24 (m, 2H), 3.44 (s, 3H), 3.46-3.47(m, 1H), 3.58-3.61 (m, 2H), 3.69 (s, 1H), 4.21 (s, 3H), 5.43 (d, J = 12.4 Hz, 1H), 6.59 (dd, J = 5.8, 2.2 Hz, 1H), 7.02 (d, J = 2.2 Hz, 1H), 7.93 (d, J = 0.8 Hz, 1H) 8.06 (d, J = 5.8 Hz, 1H), 8.22 (s, 1H), 8.65 (s, 1H), 8.67 (s, 1H), 9.87 (s, 1H). |
| I-427 | 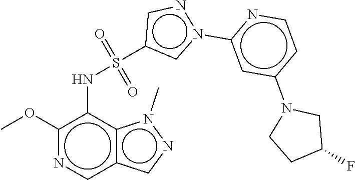 | LC-MS (ES, m/z): [M + 1]⁺ 473. ¹H NMR (400 MHz, DMSO-d₆): δ 2.35-2.24 (m, 2H), 3.44 (s, 3H), 3.46-3.47(m, 1H), 3.58-3.61 (m, 2H), 3.69 (s, 1H), 4.21 (s, 3H), 5.43 (d, J = 12.4 Hz, 1H), 6.59 (dd, J = 5.8, 2.2 Hz, 1H), 7.02 (d, J = 2.2 Hz, 1H), 7.93 (d, J = 0.8 Hz, 1H) 8.06 (d, J = 5.8 Hz, 1H), 8.22 (s, 1H), 8.65 (s, 1H), 8.67 (s, 1H), 9.87 (s, 1H). |
| I-433 | 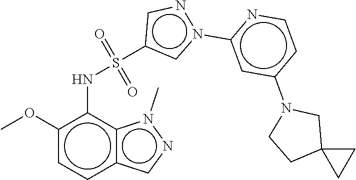 | LC-MS (ES, m/z): [M + 1]⁺ = 480. ¹H NMR (400 MHz, chloroform-d): δ 0.68 (s, 4H), 1.94-2.04 (m, 2H), 3.31-3.41 (m, 5H), 3.57 (s, 2H), 4.36-4.45 (m, 3H), 6.33 (s, 1H), 6.50 (s, 1H), 6.65 (s, 1H), 6.94 (s, 1H), 7.59-7.62 (m, 2H), 7.88-7.96 (m, 2H), 7.97-8.03 (m, 1H). |
| I-434 | 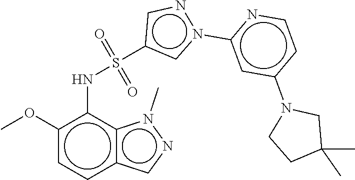 | LC-MS (ES, m/z): [M + 1]⁺ = 482. ¹H NMR (400 MHz, DMSO-d₆): δ 1.12 (s, 6H), 1.81 (t, J = 7.0 Hz, 2H), 3.14 (s, 2H), 3.32 (s, 3H), 3.41- 3.53 (m, 2H), 4.24 (s, 3H), 6.50 (dd, J = 6.0, 2.4 Hz, 1H), 6.87 (d, J = 8.8 Hz, 1H), 6.94 (d, J = 2.4 Hz, 1H), 7.67 (d, J = 8.8 Hz, 1H), 7.89 (s, 1H), 7.96-8.03 (m, 2H), 8.63 (s, 1H). |
| I-436 | 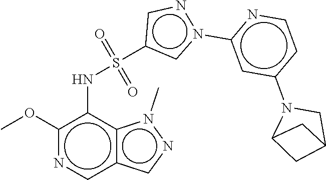 | LC-MS (ES, m/z): [M + 1]⁺ = 467. ¹H NMR (400 MHz, chloroform-d): δ 1.47 (dd, J = 2.0, 4.8 Hz, 2H), 2.07 (d, J = 1.6 Hz, 2H), 3.03 (t, J = 3.2 Hz, 1H), 3.39 (s, 2H), 3.56 (s, 3H), 4.36 (s, 3H), 4.52 (d, J = 6.8 Hz, 1H), 6.37 (s, 1H), 6.43 (dd, J = 2.4, 6.0 Hz, 1H), 7.09 (d, J = 2.4 Hz, 1H), 7.68 (s, 1H), 7.97 (d, J = 6.0 Hz, 1H), 8.04 (s, 1H), 8.53 (s, 1H), 8.80 (s, 1H). |
| I-442 | 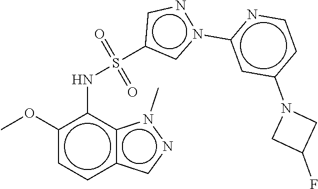 | LC-MS (ES, m/z): [M + 1]⁺ 458. ¹H NMR (400 MHz, acetonitrile- d₃): δ 3.38 (s, 3H), 4.21-4.15 (m, 2H), 4.30 (s, 3H), 4.44-4.29 (m, 2H), 5.50 (d, J =15.6 Hz, 1H), 6.40 (dd, J = 2.4, 5.6 Hz, 1H), 6.81 (d, J = 8.8 Hz, 1H), 6.94 (d, J = 2.4 Hz, 1H), 7.68 (d, J = 8.8 Hz, 1H), 7.79 (d, J = 0.8 Hz, 1H), 7.93 (s, 1H), 8.05 (d, J = 5.6 Hz, 1H), 8.63 (s, 1H). |
| I-445 | 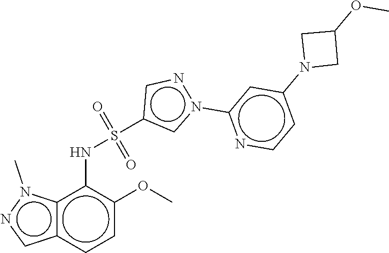 | LC-MS (ES, m/z): [M + H]⁺ = 470. ¹H NMR (400 MHz, DMSO-d₆): δ 3.27 (s, 3H), 3.32 (s, 3H), 3.84 (dd, J = 3.6, 9.2, Hz, 2H), 4.18 (dd, J = 3.6, 9.2, Hz, 2H), 4.26 (s, 3H), 4.39 (td, J = 3.2, 6.4 Hz, 1H), 6.42 (dd, J = 2.4, 5.6 Hz, 1H), 6.84 (d, J = 8.0 Hz, 1H), 6.86 (d, J = 7.2 Hz, 1H), 7.69 (d, J = 8.8 Hz, 1H), 7.90 (d, J = 0.8 Hz, 1H), 7.99 (s, 1H), 8.04 (d, J = 5.6 Hz, 1H), 8.63 (d, J = 0.8 Hz, 1H). |
| I-451 | 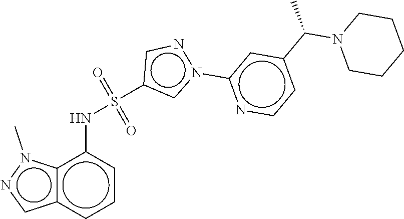 | LC-MS (ES, m/z): [M + H]⁺ = 443. ¹H NMR (400 MHz, DMSO-d₆): δ 1.38 (d, J = 6.6 Hz, 3H), 3.25 (s, 3H), 3.34 (s, 3H), 4.24 (s, 3H), 4.53 (q, J = 6.6 Hz, 1H), 6.88 (d, J = 8.8 Hz, 1H), 7.38-7.44 (m, 1H), 7.68 (d, J = 8.8 Hz, 1H), 7.92 (s, 1H), 7.99 (d, J = 4.2 Hz, 2H), 8.49 (d, J = 5.0 Hz, 1H), 8.71 (s, 1H), 9.79 (s, 1H) |
| I-452 | 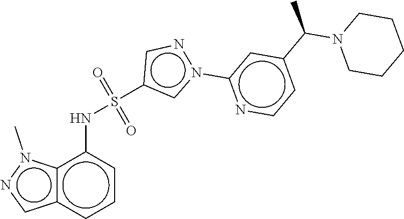 | LC-MS (ES, m/z): [M + H]⁺ = 443. ¹H NMR (400 MHz, DMSO-d₆): δ 1.38 (d, J = 6.6 Hz, 3H), 3.25 (s, 3H), 3.34 (s, 3H), 4.24 (s, 3H), 4.53 (q, J = 6.6 Hz, 1H), 6.88 (d, J = 8.8 Hz, 1H), 7.38-7.44 (m, 1H), 7.68 (d, J = 8.8 Hz, 1H), 7.92 (s, 1H), 7.99 (d, J = 4.2 Hz, 2H), 8.49 (d, J = 5.0 Hz, 1H), 8.71 (s, 1H), 9.79 (s, 1H). |
| I-453 | 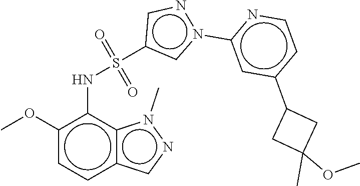 | LC-MS (ES, m/z): [M + 1]⁺ = 484. ¹H NMR (400 MHz, DMSO-d₆): δ 1.45 (s, 3H), 3.18 (s, 3H), 3.28 (s, 3H), 3.81 (d, J = 8.9 Hz, 2H), 3.90 (d, J = 8.8 Hz, 2H), 4.20 (s, 3H), 6.40 (d, J = 6.2 Hz, 1H), 6.87-6.79 (m, 2H), 7.66 (d, J = 8.9 Hz, 1H), 7.86 (s, 1H), 8.02-7.94 (m, 2H), 8.59 (s, 1H). |
| I-454 | 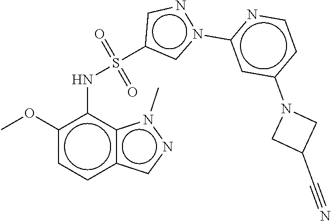 | LC-MS (ES, m/z): [M + 1]⁺ = 465. ¹H NMR (400 MHz, DMSO-d₆): δ 3.32 (d, J = 3.4 Hz, 3H), 3.93 (s, 1H), 4.04-4.28 (m, 5H), 4.34 (td, J = 8.5, 2.4 Hz, 2H), 6.48 (dd, J = 5.8, 2.2 Hz, 1H), 6.84-6.92 (m, 2H), 7.63 (d, J = 8.8 Hz, 1H), 7.90 (d, J = 2.8 Hz, 1H), 7.97 (s, 1H), 8.07- 8.12 (m, 1H), 8.63 (d, J = 1.8 Hz, 1H). |
| I-456 | 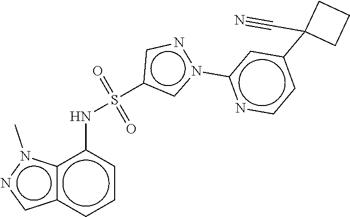 | LC-MS (ES, m/z): [M + H]⁺ = 434. ¹H NMR (400 MHz, methanol-d₄): δ 2.13-2.26 (m, 1H), 2.41-2.55 (m, 1H), 2.73-2.82 (m, 2H), 2.83-2.91 (m, J = 12.6, 6.8, 3.6 Hz, 2H), 4.38 (s, 3H), 6.76 (d, J = 7.2 Hz, 1H), 7.00-6.92 (m, 1H), 7.53 (dd, J = 5.2, 1.6 Hz, 1H), 7.66 (d, J = 8.0 Hz, 1H), 7.89 (s, 1H), 8.01 (s, 1H), 8.13 (d, J = 1.6 Hz, 1H), 8.51 (d, J = 5.2 Hz, 1H), 8.80 (d, J = 0.8 Hz, 1H). |
| I-458 | 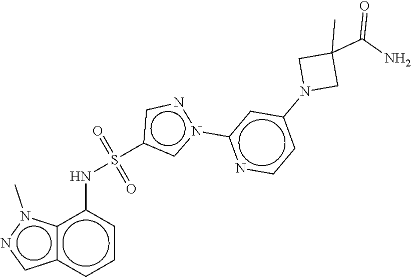 | LC-MS (ES, m/z): [M + H]⁺ 467. ¹H NMR (400 MHz, DMSO-d₆): δ 1.53 (s, 3H), 3.76 (d, J = 8.0 Hz, 2H), 4.18 (d, J = 8.0 Hz, 2H), 4.27 (s, 3H), 6.42 (dd, J = 5.6, 2.0 Hz, 1H), 6.67 (d, J = 7.2 Hz, 1H), 6.84 (d, J = 2.0 Hz, 1H), 6.97 (t, J = 7.6 Hz, 1H), 7.15 (s, 1H), 7.49 (s, 1H), 7.70 (dd, J = 8.0, 1.2 Hz, 1H), 7.94 (d, J = 0.8 Hz, 1H), 8.03 (d, J = 5.6 Hz, 1H), 8.09 (s, 1H), 8.71 (d, J = 0.8 Hz, 1H), 9.99 (s, 1H). |
| I-459 | 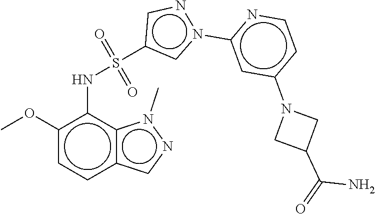 | LC-MS (ES, m/z): [M + 1]⁺ = 483. ¹H NMR (400 MHz, DMSO-d₆) 3.30-3.35 (m, 3H), 3.52 (td, J = 8.2, 7.0, 4.2 Hz, 3H), 4.02 (ddt, J = 8.2, 5.4, 2.4 Hz, 2H), 4.17 (t, J = 8.4 Hz, 2H), 4.21-4.28 (m, 3H), 6.43 (dd, J = 5.8, 2.0 Hz, 1H), 6.81-6.92 (m, 2H), 7.69 (d, J = 8.8 Hz, 1H), 7.91 (q, J = 4.2, 3.4 Hz, 1H), 7.98-8.09 (m, 2H), 8.64 (q, J = 3.7, 2.8 Hz, 1H). |
| I-460 | 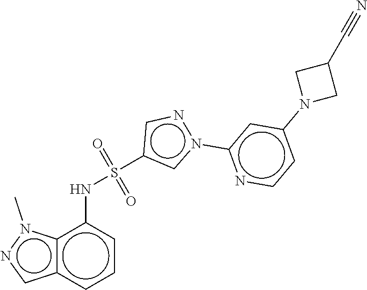 | LC-MS (ES, m/z): [M + 1]⁺ = 435. ¹H NMR (400 MHz, DMSO-d₆): δ 3.96 (tt, J = 8.9, 5.8 Hz, 1H), 4.23 (dd, J = 8.1, 5.8 Hz, 2H), 4.30 (d, J = 8.4 Hz, 5H), 6.46 (dd, J = 5.7, 2.2 Hz, 1H), 6.70 (d, J = 7.2 Hz, 1H), 6.87-6.97 (m, 2H), 7.56 (s, 1H), 7.93 (s, 1H), 8.03 (s, 1H), 8.08 (d, J = 5.8 Hz, 1H), 8.68 (s, 1H), 10.01 (s, 1H). |
| I-461 | 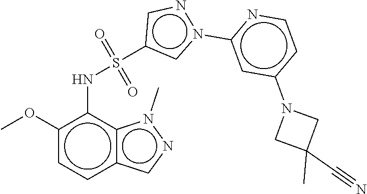 | LC-MS (ES, m/z): [M + 1]⁺ = 479. ¹H NMR (400 MHz, DMSO-d₆): δ 1.68 (s, 3H), 3.31 (s, 3H), 4.02 (d, J = 8.4 Hz, 2H), 4.23 (s, 3H), 4.39 (d, J = 8.4 Hz, 2H), 6.47 (dd, J = 5.6, 2.0 Hz, 1H), 6.87 (d, J = 8.8 Hz, 1H), 6.90 (d, J = 2.0 Hz, 1H), 7.67 (d, J = 8.8 Hz, 1H), 7.92 (s, 1H), 7.98 (s, 1H), 8.10 (d, J = 5.6 Hz, 1H), 8.63 (s, 1H), 9.75 (s, 1H). |
| I-463 | 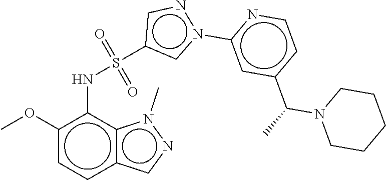 | LC-MS (ES, m/z): [M + H]⁺ = 496. ¹H NMR (400 MHz, DMSO-d₆): δ 1.34 (t, J = 8.6 Hz, 5H), 1.50 (t, J = 5.8 Hz, 4H), 2.35 (d, J = 16.8 Hz, 4H), 3.34 (s, 3H), 3.63 (d, J = 6.8 Hz, 1H), 4.25 (s, 3H), 6.88 (d, J = 8.8 Hz, 1H), 7.41 (dd, J = 5.0, 1.4 Hz, 1H), 7.68 (d, J = 8.8 Hz, 1H), 7.91 (s, 1H), 7.99 (d, J = 6.2 Hz, 2H), 8.45 (d, J = 5.0 Hz, 1H), 8.71 (s, 1H). |
| I-472 | 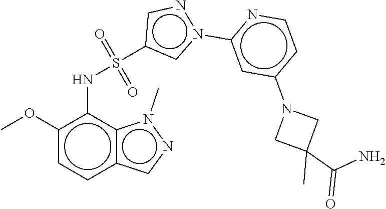 | LC-MS (ES, m/z): [M + H]⁺ = 497. ¹H NMR (400 MHz, DMSO-d₆): δ 1.53 (s, 3H), 3.31 (s, 3H), 3.75 (d, J = 8.0 Hz, 2H), 4.17 (d, J = 8.0 Hz, 2H), 4.23 (s, 3H), 6.40 (dd, J = 5.6, 2.4 Hz, 1H), 6.83 (d, J = 2.0 Hz, 1H), 6.86 (d, J = 8.8 Hz, 1H), 7.15 (s, 1H), 7.49 (s, 1H), 7.66 (d, J = 8.8 Hz, 1H), 7.90 (s, 1H), 7.97 (s, 1H), 8.03 (d, J = 5.6 Hz, 1H), 8.62 (s, 1H). |
| I-479 | 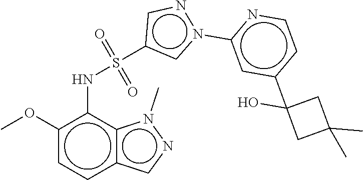 | LC-MS (ES, m/z): [M + 1]⁺ = 483. ¹H NMR (400 MHz, DMSO-d₆): δ 1.14 (s, 3H), 1.36 (s, 3H), 2.20-2.12 (m, 2H), 2.34-2.26 (m, 2H), 3.33 (s, 3H), 4.25 (s, 3H), 6.88 (d, J = 8.8 Hz, 1H), 7.51 (dd, J = 5.2, 1.6 Hz, 1H), 7.68 (d, J = 8.8 Hz, 1H), 8.00 (dd, J = 10.6, 2.2 Hz, 3H), 8.47 (d, J = 5.2 Hz, 1H), 8.71 (s, 1H). |
| I-480 | 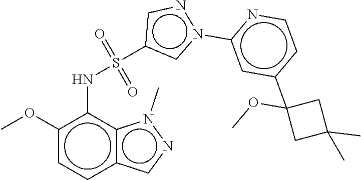 | LC-MS (ES, m/z): [M + H]⁺ = 497. ¹H NMR (400 MHz, chloroform-d): δ 1.08 (s, 3H), 1.31 (s, 3H), 2.28 (s, 4H), 2.97 (s, 3H), 3.43 (s, 3H), 4.41 (s, 3H), 6.50 (s, 1H), 6.68 (d, J = 8.8 Hz, 1H), 7.29 (dd, J = 5.1, 1.6 Hz, 1H), 7.61 (d, J = 8.8 Hz, 1H), 7.71 (s, 1H), 7.93 (d, J = 2.5 Hz, 2H), 8.39 (d, J = 5.1 Hz, 1H), 8.74 (s, 1H). |
| I-483 | 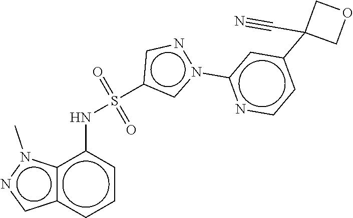 | LC-MS (ES, m/z): [M + 1]⁺ = 436. ¹H NMR (400 MHz, DMSO-d₆): δ 4.22 (s, 3H), 4.83 (d, J = 6.9 Hz, 2H), 5.16 (d, J = 6.9 Hz, 2H), 6.65 (dd, J = 7.3, 1.0 Hz, 1H), 6.90 (t, J = 7.7 Hz, 1H), 7.62 (dd, J = 8.1, 1.0 Hz, 1H), 7.71 (dd, J = 5.2, 1.8 Hz, 1H), 8.01 (d, J = 1.4 Hz, 2H), 8.14 (d, J = 1.7 Hz, 1H), 8.58 (d, J = 5.2 Hz, 1H), 8.77 (s, 1H), 10.01 (s, 1H). |

### Example 485—MALT1 Inhibition Assay

Inhibition of MALT1 activity by the presence of small molecules was evaluated using MALT-1 Fluorogenic Peptide Cleavage Assay. The assay utilizes a quenched AMC-labelled peptide that contains the MALT-1 recognition sequence and cleavage site (LRSR). MALT-1 mediated cleavage of the peptide relieves the quenching and leads to an increase in fluorescence at excitation (342 nm) and emission (441 nm).

The following reagents were obtained commercially and used to prepare standard reagent formulations as further described below: N-2-hydroxyethylpiperazine-N-2-ethane sulfonic acid (HEPES) (1 M, pH 7.5, stored at 4° C.), sodium citrate (stored at RT), tris(2-carboxyethyl) phosphine hydrochloride (T-CEP) (500 mM, stored at −20° C.), ethylenediaminetetraacetic acid (EDTA) (500 mM, stored at RT), dimethylsulfoxide (DMSO, stored at RT), 3-[(3-cholamido-propyl)dimethylammonio]-1-propane sulphate (CHAPS) (stored at RT), dimethylsulfoxide (DMSO), DMSO (Fisher Scientific, stored at RT), Avi-tagged FL MALT-1 (Pharmaron, stored at −70° C.), Ac-LRSR-AMC Peptide (SM Biochemicals, stored at −20° C.)., and ultrapure water (MILLLI-Q©).

The standard reagent formulations used in this assay were prepared and stored as follows. A 1.1 M solution of sodium citrate (161.3 g, 500 mL) was prepared and stored at room temperature. A 10% (w/v) CHAPS solution (2.0 g, 20 mL) was prepared and stored at 4° C. A 500 mM HEPES solution having pH of 6.89 was prepared from 200 mL of a 1 M HEPES solution having a pH of 7.5 using concentrated hydrochloric acid and brought to a final volume of 400 mL with MILLI-Q© H₂O. The substrate, 10 mM Ac-LRSR-AMC Peptide, was prepared (10 mg, 1.370 mL DMSO) and stored at −20° C.

Compounds were plated to provide a 2% DMSO final concentration using a ProxiPlate-384 Plus F Black 384-shallow well microplate. The assay-ready plates were equilibrated to room temperature. A reaction buffer (30 mL total volume) was prepared by combining HEPES (pH 6.89, 25 mM, 1.5 mL), sodium citrate (660 mM, 18.0 mL), T-CEP (1 mM, 0.06 mL), EDTA (0.1 mM, 0.06 mL), CHAPS (0.05%, 0.15 mL), DMSO (2%, 0.6 mL), and MILLI-Q© H₂O (10.23 mL; 9.63 mL when backfilled to 2% DMSO) followed by thorough mixing. DMSO was added only when compound plates had not been DMSO-backfilled to 2%.

MALT-1 was thawed and kept on ice. Peptide substrate was thawed on the bench under ambient conditions. A MALT-1 enzyme working stock was prepared from Avi-tagged FL MALT-1 (40 nM in a prepared reagent volume of 16.5 μL) and the reaction buffer (13.0 mL). MALT1 working stock (5 μL) was added to each well of the microplate. MALT-1 was pre-incubated with compounds for 30 minutes at room temperature.

Two substrate working stocks (Km and 10×Km) were prepared. The 1×Km substrate was prepared from Ac-LRSR-AMC Peptide (50 μM in a prepared reagent volume of 35.0 μL) and the reaction buffer (6.965 mL). The 10×Km substrate was prepared from Ac-LRSR-AMC Peptide (280 μM in a prepared reagent volume of 196.0 μL) and the reaction buffer (6.804 mL). The reaction was initiated by the addition of substrate working stock (5 μL, 50 μM) to Km plates and the addition of substrate working stock (5 μL 280 μM) to 10×Km plates. The plates were covered and incubated on the bench at room temperature for 90 minutes.

Fluorescence intensity was determined using a CLARIOstar microplate reader (BMG LABTECH) using the optimised AMC mode. Before taking readings, a 70% gain was applied on the neutral control well. Compound data were normalised to % Inhibition and used to plot sigmoidal concentration response curves to yield various parameters including IC₅₀.

The results of the MALT1 assay are reported in the table below. Compounds with an IC₅₀ less than or equal to 400 μM are designated as “A”. Compounds with an IC₅₀ greater than 400 μM and less than or equal to 1000 μM are designated as “B”. Compounds with an IC₅₀ greater than 1000 μM and less than or equal to 2500 μM are designated as “C”. Compounds with an IC₅₀ greater than 2500 μM are designated as “D”.

### Example 486—NF-KB Reporter Assay

Jurkat cells were maintained in complete RPMI 1640 media supplemented with 10% FBS. Prior to the assay, 120 nL of the serial diluted compound was added in each well of 384-well flat bottom white plate (Corning #3570) by using liquid handler Echo550. Jurkat cells were harvested by centrifugation at 120 g for 10 min and resuspended in fresh RPMI 1640 media without serum. 30 mL of cells (25,000 cells) were seeded in each well of 384-well plate containing compound. After 1 hr incubation at 37° C. in a 5% CO₂ incubator, 10 mL of diluted aCD3/aCD28/PMA (final concentration at 5 mg/mL, 5 mg/mL and 0.03 mg/mL, respectively) in RPMI 1640 without serum were added to each well. After incubation at 37° C. in 5% CO₂ for 4 hr, 30 mL of One-Glo reagent (Promega #E6120) was added into each well. The plate was left at room temperature for 5 min and the signal was measured on EnVision (PerkinElmer).

The results of the NF-kB reporter assay are reported in Table 4, below. Compounds with an IC₅₀ less than or equal to 400 nM are designated as “A”. Compounds with an IC₅₀ greater than 400 nM and less than or equal to 1000 nM are designated as “B”. Compounds with an IC₅₀ greater than 1000 nM and less than or equal to 2500 nM are designated as “C”. Compounds with an IC₅₀ greater than 2500 nM are designated as “D”. Compounds in which an IC₅₀ was not measured are designated as “N/A”.

TABLE 4
MALT1 IC₅₀ data and
NF-kB reporter assay data

| | MALT1 | NF-kB |
|---|---|---|
| Compound | Inhibition | Reporter |
| No. | IC₅₀ | Assay IC₅₀ |
| I-1 | A | A |
| I-2 | A | A |
| I-3 | A | A |
| I-4 | A | A |
| I-5 | A | A |
| I-6 | B | C |
| I-7 | A | B |
| I-8 | A | B |
| I-9 | A | B |
| I-10 | A | B |
| I-11 | C | N/A |
| I-12 | D | N/A |
| I-13 | D | N/A |
| I-14 | A | A |
| I-15 | B | A |
| I-16 | D | D |
| I-17 | A | A |
| I-18 | A | B |
| I-19 | A | A |
| I-20 | C | C |
| I-21 | A | A |
| I-22 | A | A |
| I-23 | A | A |
| I-24 | B | B |
| I-25 | A | A |
| I-26 | A | A |
| I-27 | B | D |
| I-28 | A | B |
| I-29 | A | B |
| I-30 | B | C |
| I-31 | A | B |
| I-32 | A | B |
| I-33 | A | A |
| I-34 | A | A |
| I-35 | A | A |
| I-36 | A | A |
| I-37 | A | A |
| I-38 | A | A |
| I-39 | A | A |
| I-40 | A | B |
| I-41 | A | A |
| I-42 | B | B |
| I-43 | D | D |
| I-44 | D | D |
| I-45 | D | A |
| I-47 | D | D |
| I-48 | A | D |
| I-49 | D | D |
| I-50 | B | D |
| I-51 | D | D |
| I-52 | D | C |
| I-53 | A | A |
| I-54 | A | A |
| I-55 | A | C |
| I-56 | A | A |
| I-57 | A | A |
| I-58 | D | D |
| I-59 | A | B |
| I-60 | A | A |
| I-61 | C | C |
| I-62 | A | B |
| I-63 | D | A |
| I-64 | C | D |
| I-65 | B | B |
| I-66 | D | D |
| I-67 | A | A |
| I-68 | A | A |
| I-69 | A | A |
| I-70 | A | A |
| I-71 | A | A |
| I-72 | A | A |
| I-73 | C | A |
| I-74 | A | C |
| I-75 | D | B |
| I-76 | C | A |
| I-77 | A | A |
| I-78 | A | B |
| I-79 | A | A |
| I-80 | A | C |
| I-81 | A | A |
| I-82 | A | A |
| I-83 | A | A |
| I-84 | A | A |
| I-85 | A | A |
| I-86 | D | A |
| I-87 | A | A |
| I-88 | D | A |
| I-89 | D | A |
| I-90 | A | A |
| I-91 | A | A |
| I-93 | D | A |
| I-94 | D | A |
| I-95 | D | A |
| I-96 | A | A |
| I-97 | A | A |
| I-98 | D | D |
| I-99 | D | A |
| I-100 | D | A |
| I-101 | D | A |
| I-102 | D | D |
| I-103 | D | A |
| I-104 | D | D |
| I-105 | D | C |
| I-106 | D | A |
| I-107 | D | A |
| I-108 | D | D |
| I-109 | D | A |
| I-110 | D | D |
| I-111 | D | A |
| I-112 | D | A |
| I-113 | D | A |
| I-114 | D | A |
| I-115 | A | A |
| I-116 | D | A |
| I-117 | C | A |
| I-118 | D | A |
| I-119 | D | A |
| I-120 | D | D |
| I-121 | B | A |
| I-122 | B | A |
| I-123 | D | A |
| I-124 | A | A |
| I-125 | A | A |
| I-126 | A | A |
| I-127 | A | A |
| I-128 | D | A |
| I-129 | D | B |
| I-130 | D | A |
| I-131 | D | A |
| I-132 | A | A |
| I-133 | A | A |
| I-134 | B | A |
| I-135 | B | A |
| I-136 | B | B |
| I-137 | B | A |
| I-138 | D | A |
| I-139 | D | A |
| I-140 | D | A |
| I-141 | A | A |
| I-142 | B | A |
| I-143 | C | A |
| I-144 | A | A |
| I-145 | D | A |
| I-146 | D | A |
| I-239 | D | D |
| I-240 | D | A |
| I-241 | D | D |
| I-242 | D | C |
| I-243 | D | C |
| I-244 | D | A |
| I-245 | D | A |
| I-246 | D | A |
| I-247 | D | A |
| I-248 | D | A |
| I-249 | D | A |
| I-250 | D | A |
| I-251 | D | A |
| I-252 | D | A |
| I-253 | D | B |
| I-254 | D | C |
| I-255 | D | A |
| I-256 | D | A |
| I-257 | D | C |
| I-258 | A | A |
| I-259 | D | C |
| I-260 | B | B |
| I-261 | A | A |
| I-262 | B | A |
| I-263 | B | A |
| I-264 | D | A |
| I-265 | D | A |
| I-266 | A | A |
| I-267 | D | A |
| I-268 | D | A |
| I-269 | D | A |
| I-270 | D | A |
| I-271 | D | A |
| I-272 | A | A |
| I-273 | A | A |
| I-274 | A | A |
| I-275 | D | C |
| I-276 | D | A |
| I-277 | D | A |
| I-278 | D | A |
| I-279 | B | B |
| I-280 | B | A |
| I-281 | B | A |
| I-282 | D | A |
| I-283 | D | A |
| I-284 | D | A |
| I-285 | D | B |
| I-286 | A | B |
| I-287 | D | D |
| I-288 | D | A |
| I-289 | D | D |
| I-290 | A | A |
| I-291 | A | A |
| I-292 | B | B |
| I-293 | A | B |
| I-294 | D | A |
| I-295 | D | A |
| I-296 | D | A |
| I-297 | D | A |
| I-298 | D | A |
| I-299 | A | B |
| I-300 | D | A |
| I-301 | D | A |
| I-302 | D | A |
| I-303 | D | A |
| I-304 | D | B |
| I-305 | D | B |
| I-306 | D | A |
| I-307 | D | A |
| I-308 | D | D |
| I-309 | D | D |
| I-310 | D | A |
| I-311 | D | A |
| I-312 | D | A |
| I-313 | D | A |
| I-314 | D | A |
| I-315 | B | A |
| I-316 | C | A |
| I-317 | A | A |
| I-318 | A | A |
| I-319 | D | A |
| I-320 | D | A |
| I-321 | D | A |
| I-322 | D | A |
| I-323 | B | A |
| I-324 | D | A |
| I-325 | D | D |
| I-326 | D | D |
| I-327 | A | A |
| I-328 | D | C |
| I-329 | D | C |
| I-330 | D | A |
| I-331 | D | A |
| I-332 | D | A |
| I-333 | D | A |
| I-334 | D | A |
| I-335 | D | A |
| I-336 | A | A |
| I-337 | D | D |
| I-338 | A | A |
| I-339 | A | A |
| I-340 | A | A |
| I-341 | D | A |
| I-342 | D | B |
| I-343 | A | C |
| I-344 | D | D |
| I-345 | D | B |
| I-346 | D | A |
| I-347 | D | A |
| I-348 | D | A |
| I-349 | D | A |
| I-350 | D | D |
| I-351 | D | D |
| I-352 | A | A |
| I-353 | A | A |
| I-354 | A | A |
| I-355 | D | D |
| I-356 | D | A |
| I-357 | D | D |
| I-358 | D | A |
| I-359 | D | A |
| I-360 | A | A |
| I-361 | A | A |
| I-362 | D | A |
| I-363 | D | A |
| I-364 | B | A |
| I-365 | A | A |
| I-366 | D | A |
| I-367 | D | A |
| I-368 | D | A |
| I-369 | D | A |
| I-370 | D | A |
| I-371 | D | A |
| I-372 | C | A |
| I-373 | A | A |
| I-374 | D | A |
| I-375 | D | A |
| I-376 | D | A |
| I-377 | B | A |
| I-378 | A | A |
| I-379 | D | D |
| I-380 | C | A |
| I-381 | B | A |
| I-382 | D | A |
| I-383 | D | A |
| I-384 | D | A |
| I-385 | D | A |
| I-386 | D | A |
| I-387 | D | A |
| I-388 | B | A |
| I-389 | D | A |
| I-390 | D | A |
| I-391 | D | A |
| I-392 | D | A |
| I-393 | D | D |
| I-394 | D | A |
| I-395 | D | A |
| I-396 | D | D |
| I-397 | C | A |
| I-398 | B | A |
| I-399 | D | A |
| I-400 | D | A |
| I-401 | D | A |
| I-402 | D | A |
| I-403 | D | A |
| I-404 | D | A |
| I-405 | D | A |
| I-406 | D | A |
| I-407 | D | A |
| I-408 | D | A |
| I-409 | D | A |
| I-410 | D | A |
| I-411 | A | A |
| I-412 | A | A |
| I-413 | B | A |
| I-414 | D | D |
| I-415 | D | D |
| I-416 | D | C |
| I-417 | D | A |
| I-418 | D | A |
| I-419 | D | A |
| I-420 | D | A |
| I-421 | D | A |
| I-422 | D | A |
| I-423 | D | A |
| I-424 | D | A |
| I-425 | D | A |
| I-426 | D | A |
| I-427 | D | B |
| I-428 | B | A |
| I-429 | D | A |
| I-430 | D | A |
| I-431 | D | A |
| I-432 | B | A |
| I-433 | D | A |
| I-434 | D | A |
| I-435 | D | A |
| I-436 | C | A |
| I-437 | D | A |
| I-438 | D | A |
| I-439 | D | A |
| I-440 | D | A |
| I-441 | D | A |
| I-442 | C | A |
| I-443 | C | A |
| I-444 | D | A |
| I-445 | D | A |
| I-446 | D | A |
| I-447 | D | B |
| I-448 | D | A |
| I-449 | C | A |
| I-450 | D | A |
| I-451 | D | A |
| I-452 | D | A |
| I-453 | D | B |
| I-454 | D | B |
| I-455 | D | C |
| I-456 | D | A |
| I-457 | B | A |
| I-458 | D | D |
| I-459 | D | D |
| I-460 | D | A |
| I-461 | D | B |
| I-462 | D | A |
| I-463 | D | B |
| I-464 | D | B |
| I-465 | D | C |
| I-466 | D | D |
| I-467 | D | D |
| I-468 | D | D |
| I-469 | D | A |
| I-470 | B | A |
| I-471 | D | D |
| I-472 | D | C |
| I-473 | D | D |
| I-474 | C | B |
| I-475 | D | D |
| I-476 | A | A |
| I-477 | D | A |
| I-478 | D | A |
| I-479 | D | A |
| I-480 | D | A |
| I-481 | D | A |
| I-482 | B | A |
| I-483 | D | A |
| I-484 | D | A |
| I-485 | B | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 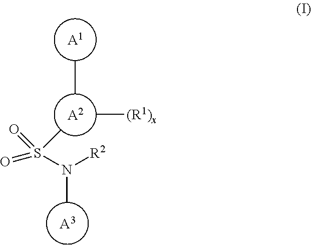
or a pharmaceutically acceptable salt thereof; wherein:
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2, or 3 heteroatoms independently selected from oxygen, nitrogen, and sulfur, a 5-6 membered partially unsaturated heterocyclyl containing 1 nitrogen atom, or a 5-6 membered deuteroheteroaryl containing 1 nitrogen atom, wherein the heteroaryl, saturated heterocyclyl, and deuteroheteroaryl are substituted with n occurrences of R⁶, and wherein the partially unsaturated heterocyclyl is substituted with n occurrences of R⁶ and 1 occurrence of oxo;
A² is pyrazolylene or 1,2,3-triazolylene;
A³ is 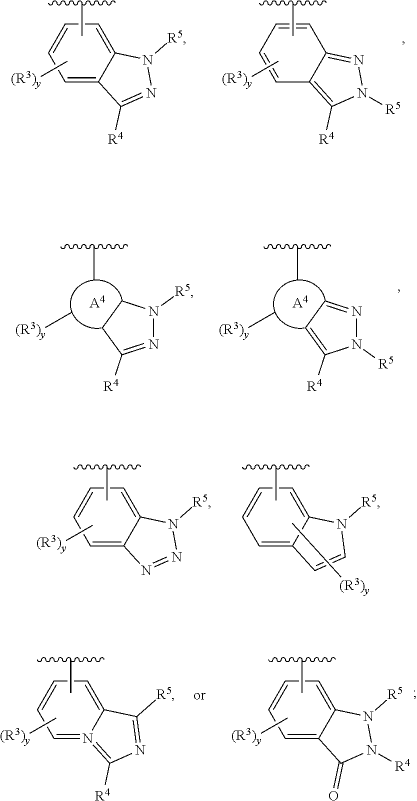
A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is C₁ alkyl, C₂ alkyl, C₁₋₄ haloalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), C₁₋₆ hydroxyalkyl, —C(O)R¹⁰, —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, C₁₋₄ alkoxyl, C₁₋₄ alkyl, or deuterium;
R⁶ represents independently for each occurrence halo, hydroxyl, C₁₋₆ alkyl, C₁₋₆ haloalkyl, cyano, C₁₋₆ alkoxyl, C₁₋₆ hydroxyalkoxyl, —(C₃₋₇ cycloalkyl substituted with q occurrences of R¹²), —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, C₁₋₆ hydroxyalkyl, —(C₁₋₄ alkylene)-(C₁₋₆ alkoxyl), —(C₁₋₄ alkylene)-CN, C₂₋₄ alkenyl, C₂₋₄ haloalkenyl, —(C₀₋₄ alkylene)-(N(R¹3)(R¹⁴)), —(C₀₋₄ alkylene)-(3-7 membered saturated or partially unsaturated monocyclic heterocyclyl containing 1 or 2 heteroatoms independently selected from oxygen and nitrogen), or a 6-7 membered saturated bicyclic heterocyclyl containing 1 nitrogen atom, wherein the monocyclic heterocyclyl and bicyclic heterocyclyl are substituted with 0, 1, or 2 occurrences of R¹²;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, C₁₋₆ hydroxyalkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
R¹² represents independently for each occurrence C₁₋₆ alkyl, C₁₋₆ alkoxyl, halo, hydroxyl, C₁₋₆ haloalkyl, oxo, cyano, or —C(O)—(C₁₋₄ alkyl); or two R¹² groups taken together with the carbon atom to which they are attached form a 3-7 membered saturated carbocyclic ring;
R¹³ and R¹⁴ are independently hydrogen or C₁₋₄ alkyl;
n, m, x, and y are independently 0, 1, or 2; and
q is 0, 1, 2, or 3.

2. The compound of claim 1, wherein the compound is a compound of Formula I.

3. The compound of claim 1, wherein the compound is a compound of Formula I-1: 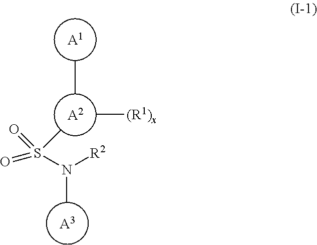
or a pharmaceutically acceptable salt thereof; wherein:
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is pyrazolylene or 1,2,3-triazolylene; 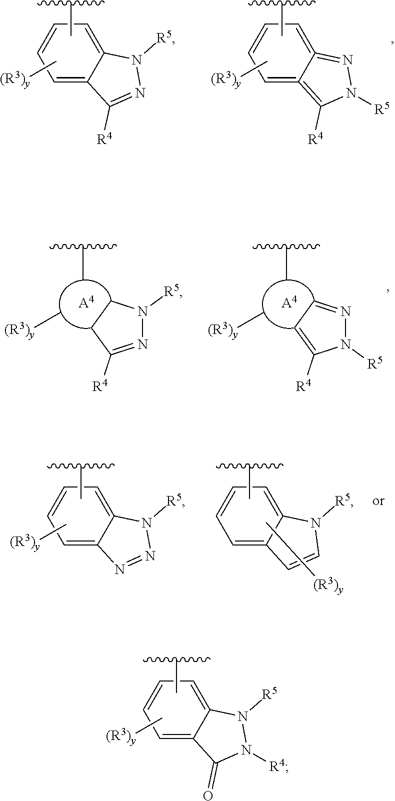
A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is C₁ alkyl, C₂ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, C₃₋₇ halocycloalkyl, C₃₋₇ hydroxycycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, —S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), or —N(R⁸)S(O₂)R¹⁰;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

4-6. (canceled)

7. The compound of claim 1, wherein the compound is a compound of Formula Ia or a pharmaceutically acceptable salt thereof:

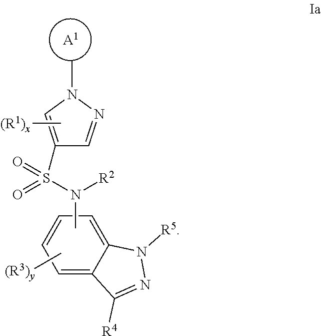

8. The compound of claim 1, wherein the compound is a compound of Formula Ib or Ic or a pharmaceutically acceptable salt thereof:

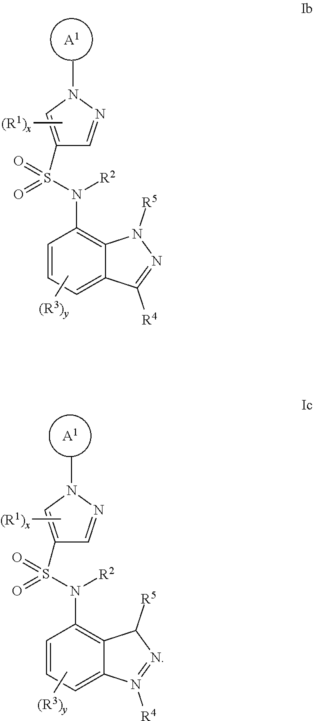

9. The compound of claim 8, wherein y is 1.

10. The compound of claim 1, wherein the compound is a compound of Formula Id or Ie or a pharmaceutically acceptable salt thereof:

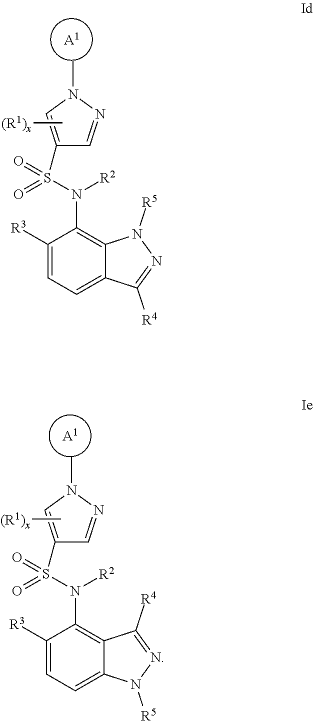

11. The compound of claim 1, wherein the compound is a compound of Formula If or a pharmaceutically acceptable salt thereof:

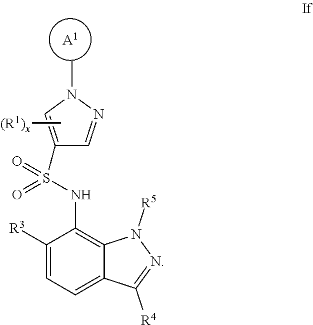

12. The compound of claim 11, wherein x is 0.

13-18. (canceled)

19. The compound of claim 11, wherein A¹ is a 6-membered heteroaryl containing 1, 2, or 3 heteroatoms independently selected from oxygen, nitrogen, and sulfur, wherein the heteroaryl is substituted with n occurrences of R⁶.

20. The compound of claim 11, wherein A¹ is pyridinyl, pyrimidinyl, pyridazinyl, or pyrazinyl, each of which is substituted with n occurrences of R⁶.

21. The compound of claim 11, wherein A¹ is pyridinyl substituted with n occurrences of R⁶.

22. The compound of claim 11, wherein A¹ is 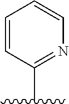 [SMILES: *c1ccccn1]
substituted with n occurrences of R⁶.

23. The compound of claim 22, wherein n is 1.

24. (canceled)

25. The compound of claim 11, wherein A¹ is

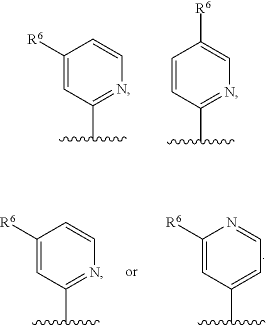

26-27. (canceled)

28. The compound of claim 23, wherein R⁶ is C₁₋₆ haloalkyl.

29. The compound of claim 23, wherein R⁶ is —CF₃.

30-34. (canceled)

35. The compound of claim 11, wherein R⁵ is C₂ alkyl.

36. The compound of claim 11, wherein R⁵ is methyl.

37. The compound of claim 11, wherein R⁴ is hydrogen.

38-43. (canceled)

44. The compound of claim 1, wherein the compound is a compound of Formula Iq or a pharmaceutically acceptable salt thereof:

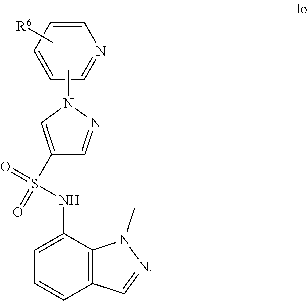

45. The compound of claim 1, wherein the compound is a compound of Formula Ir or a pharmaceutically acceptable salt thereof:

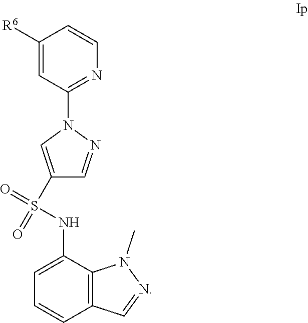

46. The compound of claim 45, wherein R⁶ is C₁₋₆ haloalkyl, —C₁₋₆ hydroxyalkyl, —(C₁₋₄ alkylene)-CN, or a 3-7 membered saturated or partially unsaturated monocyclic heterocyclyl containing 1 or 2 heteroatoms independently selected from oxygen and nitrogen, wherein the monocyclic heterocyclyl is substituted with 1 occurrence of R¹².

47. The compound of or claim 45, wherein R⁶ is C₁₋₃ haloalkyl.

48. The compound of or claim 45, wherein R⁶ is —C₂₋₆ hydroxyalkyl.

49-51. (canceled)

52. A compound represented by Formula II: 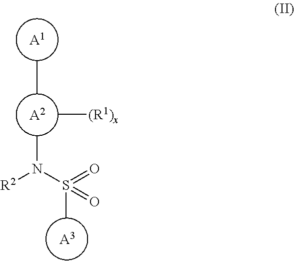
or a pharmaceutically acceptable salt thereof; wherein:
A¹ is a 5-6 membered heteroaryl containing 1, 2, or 3 heteratoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteratoms independently selected from oxygen, nitrogen, and sulfur, wherein the heteroaryl and heterocyclyl are substituted with n occurrences of R⁶;
A² is pyrazolylene or 1,2,3-triazolylene;
A³ is 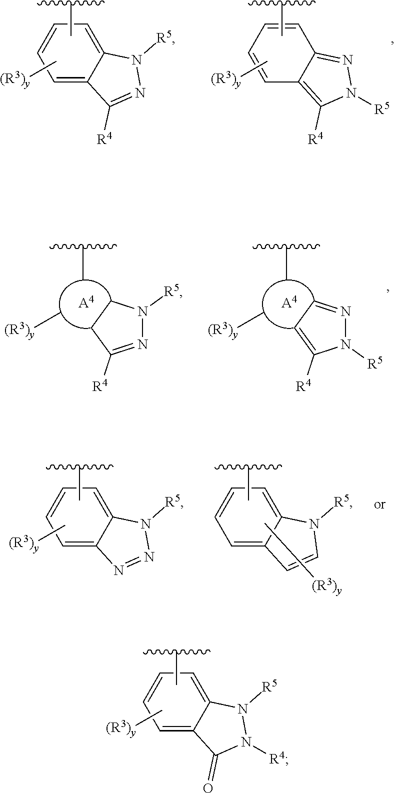
A⁴ is a 6-membered aromatic ring containing 1 nitrogen atom;
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, —(C₀₋₄ alkylene)-CN, cyano, C₂₋₄ alkynyl, —N(R⁸)(R⁹), —CO₂R¹⁰, —C(O)N(R⁸)(R⁹), —N(R⁸)C(O)R¹⁰, or —S(O₂)R¹⁰;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, —N(R⁸)(R⁹), —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteratoms heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or (C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
y is 0, 1, or 2; and
n, m, and x are independently 0, 1, or 2.

53-65. (canceled)

66. A compound in Table 1 or 2, or a pharmaceutically acceptable salt thereof.

67. A pharmaceutical composition comprising a compound of claim 1 and a pharmaceutically acceptable carrier.

68. A method for treating a disease or condition mediated by MALT1, comprising administering to a subject in need thereof a therapeutically effective amount of a compound of claim 1 to treat the disease or condition.

69-71. (canceled)

72. The method of claim 68, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

73. The method of claim 68, wherein said disease or condition mediated by MALT1 is cancer.

74. The method of claim 68, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

75. The method of claim 68, wherein the cancer is a lymphoma or leukemia.

76. The method of claim 68, wherein the cancer is a B-cell lymphoma or chronic myelocytic leukemia.

77. The method of claim 68, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

78. The method of claim 68, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, Hashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

79. (canceled)

80. The method of claim 68, wherein the subject is a human.

81. A method of inhibiting the activity of MALT1, comprising contacting a MALT1 with an effective amount of a compound of claim 1 to inhibit the activity of said MALT1.
